(19) United States
(12) Patent Application Publication
(10) Pub. No.: US 2017/0226570 A1
(43) Pub. Date: Aug. 10, 2017

(54) DNA METHYLATION MARKERS FOR OVERGROWTH SYNDROMES

(71) Applicant: The Hospital for Sick Children, Toronto (CA)

(72) Inventors: Rosanna Weksberg, Toronto (CA); Sanaa Choufani, Maple (CA); Daria Grafodatskaya, Mississauga (CA)

(21) Appl. No.: 15/518,856

(22) PCT Filed: Aug. 28, 2015
(86) PCT No.: PCT/CA2015/050828

§ 371 Date: Apr. 13, 2017

Related U.S. Application Data
(60) Provisional application No. 62/065,374, filed on Oct. 17, 2014.
(60) Provisional application No. 62/076,765, filed on Nov. 7, 2014.

(51) Int. Cl. C12Q 1/68

(52) U.S. Cl. CPC C12Q 1/6827; C12Q 1/6806; C12Q 2600/154

(57) ABSTRACT

The present disclosure provides epigenetic signatures, comprising genomic CpG dinucleotide sequences, genes, and/or genomic regions, which are differentially methylated in individuals with Sotos syndrome relative to non-Sotos syndrome controls, and their use in methods and kits for detecting and/or screening for Sotos syndrome, or the likelihood of Sotos syndrome. The present disclosure also provides epigenetic signatures, comprising genomic CpG dinucleotide sequences, genes, and/or genomic regions, which are differentially methylated in individuals with Weaver syndrome relative to non-Weaver syndrome controls, and their use in methods and kits for detecting and/or screening for Weaver syndrome, or the likelihood of Weaver syndrome.

### RELATED APPLICATION

This application claims the benefit of priority to U.S. Provisional Applications Nos. 62/065,374 filed Oct. 17, 2014 and 621076,765 filed Nov. 7, 2014, respectively. The contents of which are incorporated herein by reference in their entirety.

### FIELD

The disclosure relates to methods and kits for detecting and/or screening for Sotos syndrome (SS), or an increased likelihood of SS, in a human subject. The disclosure also relates to methods and kits for detecting and/or screening for Weaver syndrome (WVS), or an increased likelihood of WVS, in a human subject.

### INTRODUCTION

Sotos syndrome (SS; OMIM 117550) is one of the most common and well-characterized overgrowth syndromes. It is characterized by pre- and postnatal overgrowth, advanced bone age, distinctive facial gestalt and a variety of neurodevelopmental problems including intellectual disability and behavioural difficulties [Sotos et al, 1964]. Mutations in the nuclear receptor SET (su(var)3-9, enhancer-of-zeste, trithorax) domain containing protein-1 (NSD1) gene are found in patients with Sotos syndrome [Baujat and Cormier-Daire 2007; Kurotaki et al, 2002; Tatton-Brown and Rahman 2007]. NSD1 is one of a large number of genes that developmentally regulate the epigenome. NSD1 encodes a histone lysine methyltransferase [Stec et al, 1998] and binds near various promoter elements to regulate transcription via interactions with H3K36 methylation and RNA polymerase II.

Weaver syndrome (WVS; OMIM 277590), first described in 1974, is an overgrowth syndrome characterized by tall stature, advanced bone age, characteristic facial features, variable intellectual disability and a range of associated clinical features including, poor coordination, soft doughy skin, camptodactyly of the fingers or toes, umbilical hernia, abnormal tone, and hoarse low cry [Weaver 1974; Tatton-Brown et al 2013]. Mutations in the enhancer of zeste (drosophila, homolog 2) (EZH2) gene are found in patients with Weaver syndrome [Gibson et al 2012; Tatton-Brown et al 2011]. EZH2 encodes a histone methyltransferase involved in chromatin remodeling and transcriptional repression. EZH2 in combination with EED (Embryonic Ectoderm Development protein, mouse, homolog of) and SUZ12 (Suppressor of Zeste 12, drosophila, homolog of) forms the polycomb repressive complex (PRC2) which catalyzes the methylation of lysine residue 27 of histone 3 (H3K27).

Individuals with Weaver syndrome present with variable, somewhat non-specific clinical features that overlap with other overgrowth syndromes, most notably Sotos syndrome. Establishing a clinical diagnosis of Weaver syndrome is therefore challenging.

Targeted gene sequencing is the cornerstone of current molecular diagnostics for a multitude of genetic disorders, including Sotos syndrome. However, this diagnostic approach carries inherent challenges including inadequate power to identify all pathogenic mutations, in that the functional impact of non-synonymous variants (variants of unknown significance or “VUS”) remains unclear. Several different pathogenicity prediction algorithms have been developed; however, these tools often provide incongruent results [Thusberg et al, 2011]. Accordingly, there are a large number/percentage of cases for which the classification of single nucleotide variants into pathogenic and benign is problematic. Further, even when a pathogenic mutation is identified, little is known about the molecular and developmental pathophysiology of these disorders making it difficult to design targeted/specific therapies.

Accordingly, there is a need for robust and cost-effective tests capable of identifying Sotos syndrome and Weaver syndrome cases with high specificity and sensitivity. These tests may also be used to identify Sotos and Weaver syndrome in individuals carrying variants of unknown significance.

### SUMMARY

The present disclosure provides DNA methylation markers which are capable of differentiating Sotos syndrome (SS) cases carrying a pathogenic NSD1 mutation from non-Sotos syndrome (non-SS) controls, including distinguishing Sotos syndrome cases from individuals carrying a benign NSD1 variant (benign variant as referred to herein means a variant in NSD1 gene that does not alter protein function). The DNA methylation markers and the methods of their use described herein may provide useful alternative or supplementary diagnostics to currently available methods of detecting and/or screening for SS, or likelihood of SS.

In an aspect, there is provided a method of detecting and/or screening for Sotos syndrome (SS), or an increased likelihood of SS, in a human subject, comprising determining a sample DNA methylation profile from a sample of DNA from said subject, said sample profile comprising the methylation level of at least one, optionally at least 7, at least 10, at least 17, at least 25, at least 41, at least 57, at least 87, at least 124, at least 156, at least 220, at least 287, at least 399, at least 549, at least 726, or all CpG loci from (i) Table 1 and/or (ii) associated CpG loci residing within 300 nucleotides, optionally within 150 nucleotides, of the CpG loci of (i).

The method further comprises determining the level of similarity of said sample profile to one or more control profiles, wherein (i) a high level of similarity of the sample profile to an SS specific control profile; (ii) a low level of similarity to a non-SS control profile; and/or (iii) a higher level of similarity to a SS specific control profile than to a non-SS control profile indicates the presence of, or an increased likelihood of, SS.

In an embodiment, the CpG loci comprise (i) CpG loci from Table 1 having an absolute SS delta-beta value ≧0.4, optionally ≧0.42, ≧0.44, ≧0.46, ≧0.5, ≧0.52, ≧0.54, ≧0.56, ≧0.58, ≧0.6, ≧0.62, ≧0.64, or ≧0.66; and/or (ii) associated CpG loci residing within 300 nucleotides, optionally within 150 nucleotides, of the CpG loci of (i).

In another aspect, there is provided a method of detecting and/or screening for Sotos syndrome (SS), or an increased likelihood of SS, in a human subject, comprising:

determining a sample methylation profile from a sample of DNA from said subject, said sample profile comprising the methylation level of CpG loci, wherein the CpG loci are the foci from Table 1 having an absolute SS delta-beta value ≧0.4; and

determining the level of similarity of said sample profile to one or more control profiles, wherein (i) a high level of similarity of the sample profile to an SS specific control profile; (ii) a low level of similarity to a non-SS control profile; and/or (iii) a higher level of similarity to a SS specific control profile than to a non-SS control profile indicates the presence of, or an increased likelihood of, SS.

In an embodiment, the CpG loci comprise CpG loci from Table 1 having an absolute SS delta-beta value ≧0.4, optionally ≧0.42, ≧0.44, ≧0.46, ≧0.48, ≧0.5, ≧0.52, ≧0.54, ≧0.56, ≧0.58, ≧0.6, ≧0.62, ≧0.64 or ≧0.66.

In another embodiment, determining the sample methylation profile comprises the steps:

a) providing the sample comprising genomic DNA from the subject;

b) optionally, isolating DNA from the sample;

c) optionally, treating DNA from the sample with bisulfite for a time and under conditions sufficient to convert non-methylated cytosines to uracils;

d) optionally, amplifying the DNA; and

e) determining the methylation level at the CpG loci by means of bisulfite sequencing, pyrosequencing, methylation-sensitive single-strand conformation analysis (MS-SSCA), high resolution melting analysis (HRM), combined bisulfite restriction analysis (COBRA), methylation-sensitive single nucleotide primer extension (MS-SnuPE), base-specific cleavage/MALDI-TOF, methylation-specific PCR (MSP), methylation-sensitive restriction enzyme-based methods, microarray-based methods, whole-genome bisulfite sequencing (WGBS, MethylC-seq or BS-seq), reduced-representation bisulfite sequencing (RRBS), and/or enrichment-based methods such as MeDIP-seq, MBD-seq, or MRE-seq.

In one embodiment, level of similarity is indicated by a correlation coefficient. In another embodiment, the correlation coefficient is a linear correlation coefficient, optionally a Pearson correlation coefficient or a Spearman correlation coefficient.

In another embodiment, a high level of similarity to the control profile is indicated by a Pearson correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25.

In another embodiment, a higher level of similarity to the SS specific control profile than to the non-SS control profile is indicated by a higher correlation value computed between the sample profile and the SS specific control profile than an equivalent correlation value computed between the sample profile and the non-SS control profile, optionally wherein the correlation value is a correlation coefficient, optionally a Pearson correlation coefficient or a Spearman correlation coefficient.

In an embodiment, the methylation level is measured as a value.

In another embodiment, a Sotos Syndrome Score (SS score) is calculated according to following formula:

SS score(B)=r (B, SS profile)−r(B, non-SS profile)

where r is a Pearson correlation coefficient, and B is a vector of DNA methylation levels across the selected CpG loci in the sample. In another embodiment, determining the sample methylation profile comprises contacting the DNA with at least one agent that provides for determination of a CpG methylation status of at least one, optionally all, of the selected CpG loci, wherein the agent comprises an oligonucleotide-immobilized substrate comprising a plurality of capture probes, each capture probe comprising a pair of capture oligonucleotides, wherein the capture oligonucleotide pairs comprise (a) an oligonucleotide comprising nucleotide sequence complementary to or identical to a nucleotide sequence of genomic DNA comprising a selected CpG, and (b) an oligonucleotide comprising nucleotide sequence complementary to or identical to a nucleotide sequence of genomic DNA comprising the same selected CpG locus of (a), in which the cytosine residue of the CpG locus is replaced with a thymine residue.

In yet another embodiment, the contacting is under hybridizing conditions.

In an embodiment, the methylation levels of the selected loci of at least one control profile is derived from one or more samples, optionally from historical methylation data for a patient or pool of patients.

In another embodiment, the non-SS control profile comprises methylation levels for the selected CpG loci listed in Table 1. In yet another embodiment, the SS specific control profile comprises DNA methylation levels for the selected CpG loci listed in Table 1. In an embodiment, the methylation levels of associated CpG loci not listed in Table 1 is assumed to be equivalent to the methylation level of a CpG loci listed in Table 1 with which the CpG loci is associated.

In an embodiment, the sample is derived from blood, fibroblast tissue, buccal tissue, lymphoblastoid cell line, saliva or a prenatal sample. The prenatal sample is optionally a CVS, placenta, circulating fetal DNA and/or amniotic fluid sample. In another embodiment, the sample is derived from a tissue biopsy.

In another embodiment, the human subject is a fetus.

Another aspect provides a method of detecting and/or screening for Sotos syndrome (SS), or an increased likelihood of SS, in a human subject, comprising determining the methylation level of at least one, optionally at least 6, at least 12, at least 17, at least 18, at least 24, at least 33, at least 49, at least 67, at least 72, at least 101, at least 127, at least 164, at least 212, at least 275, or all the genes from Table 1 in a sample comprising genomic DNA from the subject, wherein a difference in methylation levels of genes from Table 1, relative to a non-SS control profile is indicative of SS, or an increased likelihood of SS, in the subject.

Another aspect provides method of detecting and/or screening for Sotos syndrome (SS), or an increased likelihood of SS, in a human subject, comprising:

determining a sample methylation profile from a sample comprising DNA from said subject, same sample profile comprising the methylation level of at least one, optionally at least 6, at least 12, at least 17, at least 18, at least 24, at least 33, at least 49, at least 67, at least 72, at least 101, at least 127, at least 164, at least 212, at least 275, or all the genes from Table 1; and

determining the level of similarity of said sample profile to one or more control profiles, wherein (i) a high level of similarity of the sample profile to a SS specific control profile; (ii) a low level of similarity to a non-SS control profile; and/or (ii) a higher level of similarity to a SS control profile than a non-SS control profile indicates the presence of, or an increased likelihood of, SS.

In one embodiment, the methylation level is measured as a 0-value.

In another embodiment, hypermethylation is indicated by the gene having a significantly higher methylation beta value in the SS specific control profile compared to the non-SS control profile and hypomethylation is indicated by the gene having a significantly lower methylation beta value in the SS specific control profile compared to the non-SS control profile.

In another embodiment, the sample is derived from blood, fibroblast tissue, buccal tissue, lymphoblastoid cell line, saliva or a prenatal sample, optionally a CVS, placenta, circulating fetal DNA and/or amniotic fluid sample.

In another embodiment, the human subject is a fetus.

Another aspect provides a method of assigning a course of management for an individual with Sotos syndrome (SS), or an increased likelihood of SS, comprising:

a) identifying an individual with SS or an increased likelihood of SS, according to the methods disclosed herein; and

b) assigning a course of management for SS and/or symptoms of a SS, comprising i) testing for at least one medical condition associated with SS and ii) applying an appropriate medical intervention based on the results of the testing.

In one embodiment, the medical condition is selected from congenital heart defects, congenital renal abnormalities, hearing problems, vision problems, scoliosis, seizure activity, developmental/learning disabilities, behavioural problems and neuropsychological issues.

Another aspect of the disclosure provides a kit for detecting and/or screening for Sotos syndrome, or an increased likelihood of SS, in a sample, comprising:

(a) at least one detection agent for determining the methylation level of:
i) at least one, optionally at least 7, at least 10, at least 17, at least 25, at least 41, at least 57, at least 87, at least 124, at least 156, at least 220, at least 287, at least 399, at least 549, at least 726, or all CpG loci selected from Table 1, and/or
ii) at least one, optionally at least 6, at least 12, at least 17, at least 18, at least 24, at least 33, at least 49, at least 67, at least 72, at least 101, at least 127, at least 164, at least 212, at least 275, or all the genes from Table 1; and

(b) instructions for use.

In an embodiment, the kit further comprises bisulfite conversion reagents, methylation-dependent restriction enzymes, methylation-sensitive restriction enzymes, PCR reagents, probes and/or primers.

In an embodiment, the kit further comprises a computer-readable medium that causes a computer to compare methylation levels from a sample at the selected CpG loci to one or more control profiles and computes a correlation value between the sample and control profile. In an embodiment, the computer readable medium obtains the control profile from historical methylation data for a patient or pool of patients known to have, or not have, Sotos syndrome. In some embodiments, the computer readable medium causes a computer to update the control profile based on the testing results from the testing of a new patient.

The present disclosure also provides DNA methylation markers which are capable of differentiating Weaver syndrome (WVS) cases carrying a pathogenic EZH2 mutation from non-Weaver syndrome (non-WVS) controls, including distinguishing Weaver syndrome cases from individuals carrying a benign EZH2 variant (benign variant as referred to herein means a variant in EZH2 gene that does not alter protein function). The DNA methylation markers and the methods of their use described herein may provide useful alternative or supplementary diagnostics to currently available methods of detecting and/or screening for WVS, or likelihood of WVS.

Accordingly, another aspect of the disclosure provides a method of detecting and/or screening for Weaver syndrome (WVS), or an increased likelihood of WVS, in a human subject, comprising:

determining a sample methylation profile from a sample comprising DNA from said subject, said sample profile comprising the methylation level of at least one, optionally at least 3, at least 5, at least 10, at least 20, at least 30, at least 40, at least 50 or all CpG loci from (i) Table 8 and/or (ii) associated CpG loci residing within 300 nucleotides, optionally within 150 nucleotides, of the CpG loci of (i); and

determining the level of similarity of said sample profile to one or more control profiles, wherein (i) a high level of similarity of the sample profile to an WVS specific control profile; (ii) a low level of similarity to a non-WVS control profile; and/or (iii) a higher level of similarity to a WVS specific control profile than to a non-WVS control profile indicates the presence of, or an increased likelihood of, WVS.

In one embodiment, determining the sample methylation profile comprises the steps:

a) providing the sample comprising genomic DNA from the subject;

b) optionally, isolating DNA from the sample;

c) optionally, treating DNA from the sample with bisulfite for a time and under conditions sufficient to convert non-methylated cytosines to uracils;

d) optionally, amplifying the DNA; and

e) determining the methylation level at the CpG loci by means of bisulfite sequencing, pyrosequencing, methylation-sensitive single-strand conformation analysis (MS-SSCA), high resolution melting analysis (HRM), combined bisulfite restriction analysis (COBRA), methylation-sensitive single nucleotide primer extension (MS-SnuPE), base-specific cleavage/MALDI-TOF, methylation-specific PCR (MSP), methylation-sensitive restriction enzyme-based methods, microarray-based methods, whole-genome bisulfite sequencing (WGBS, MethylC-seq or BS-seq), reduced-representation bisulfite sequencing(RRBS), and/or enrichment-based methods such as MeDIP-seq, MBD-seq, or MRE-seq.

In another embodiment, a high level of similarity to the control profile is indicated by a correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25.

In another embodiment, the level of similarity to the WVS specific profile than to the non-WVS control profile is indicated by a higher correlation value computed between the sample profile and the WVS specific profile than an equivalent correlation value computed between the sample profile and the non-WVS control profile, optionally wherein the correlation value is a correlation coefficient.

In another embodiment, the correlation coefficient is a linear correlation coefficient, optionally a Pearson correlation coefficient.

In another embodiment, methylation level is measured as a β-value.

In another embodiment, a Weaver Syndrome Score (WVS score) is calculated according to following formula:

WVS score(B)=r(B, WVS profile)−r(B, non-WVS profile)

where r is a Pearson correlation coefficient, and B is a vector of DNA methylation levels across the selected CpG loci in the sample.

In another embodiment, determining the profile of methylated DNA from the subject comprises contacting the DNA with at least one agent that provides for determination of a CpG methylation status of at least one, optionally all, of the selected CpG loci, wherein the agent comprises an oligonucleotide-immobilized substrate comprising a plurality of capture probes, each capture probe comprising a pair of capture oligonucleotides, wherein the capture oligonucleotide pairs comprise (a) an oligonucleotide comprising nucleotide sequence complementary to or identical to a nucleotide sequence of genomic DNA comprising a selected CpG loci, and (b) an oligonucleotide comprising nucleotide sequence complementary to or identical to a nucleotide sequence of genomic DNA comprising the same selected CpG loci of (a), in which the cytosine residue of the CpG loci is replaced with a thymine residue.

Optionally, the contacting is under hybridizing conditions.

In another embodiment, the methylation levels of the selected loci of at least one control profile is derived from one or more samples, optionally from historical methylation data for a patient or pool of patients.

In another embodiment, the non-WVS control profile comprises methylation levels for the selected CpG loci listed in Table 8.

In another embodiment, the WVS specific control profile comprises methylation levels for the selected CpG loci listed in Table 8.

Optionally, the methylation level of a selected CpG locus not listed in Table 8 is assumed to be equivalent to the methylation level of a CpG locus listed in Table 8 with which the selected DNA CpG locus is associated.

In another embodiment, the sample is derived from blood, fibroblast tissue, buccal tissue, lymphoblastoid cell line, saliva or a prenatal sample, optionally a CVS, placenta, circulating fetal DNA and/or amniotic fluid sample.

In yet another embodiment, the human subject is a fetus.

Another aspect of the disclosure provides a method of detecting and/or screening for Weaver syndrome (WVS), or an increased likelihood of WVS, in a human subject, comprising:

determining a sample methylation profile from a sample comprising DNA from said subject, same sample profile comprising the methylation level of at least one, optionally at least 2, at least 4, at least 6, or all the genes from Table 8; and

determining the level of similarity of said sample profile to one or more control profiles, wherein (i) a high level of similarity of the sample profile to a WVS specific control profile; (ii) a low level of similarity to a non-WVS control profile; and/or (ii) a higher level of similarity to a WVS control profile than a non-WVS control profile indicates the presence of, or an increased likelihood of, WVS,

In one embodiment, determining the methylation levels of the selected genes comprises the steps:

a) providing the sample comprising genomic DNA from the subject;

b) optionally, isolating DNA from the sample;

c) optionally, treating DNA from the sample with bisulfite for a time and under conditions sufficient to convert non-methylated cytosines to uracils;

d) optionally, amplifying the DNA; and

e) determining the methylation status at the selected genes by means of bisulfite sequencing, pyrosequencing, methylation-sensitive single-strand conformation analysis (MS-SSCA), high resolution melting analysis (HRM), combined bisulfite restriction analysis (COBRA), methylation-sensitive single nucleotide primer extension (MS-SnuPE), base-specific cleavage/MALDI-TOF, methylation-specific PCR (MSP), methylation-sensitive restriction enzyme-based methods, microarray-based methods, whole-genome bisulfite sequencing (WGBS, MethylC-seq or BS-seq), reduced-representation bisulfite sequencing (RRBS), and/or enrichment-based methods such as MeDIP-seq, MBD-seq, or MRE-seq.

In another embodiment, the methylation level is measured as a β-value.

In another embodiment, hypermethylation is indicated by the selected gene having a significantly higher methylation beta vale in the WVS specific control profile compared to the non-WVS control profile and hypomethylation is indicated by the selected gene having a significantly lower methylation beta value in the WVS specific control profile compared to the non-WVS control profile.

In another embodiment, the sample is derived from blood, fibroblast tissue, buccal tissue, lymphoblastoid cell line, saliva or a prenatal sample, optionally a CVS, placenta, circulating fetal DNA and/or amniotic fluid sample.

In another embodiment, the human subject is a fetus.

The present disclosure also provides a method of determining a course of management for an individual with Weaver syndrome (WVS), or an increased likelihood of WVS, comprising:

a) identifying an individual with WVS or an increased likelihood of WVS, according to the methods described herein; and

b) assigning a course of management for WVS and/or symptoms of a WVS, comprising i) testing for at least one medical condition associated with SS and ii) applying an appropriate medical intervention based on the results of the testing.

In one embodiment, the medical condition is selected from scoliosis, camptodactyly, tumor growth, developmental/learning disabilities, intellectual disabilities, hypotonia and ligamentous laxity.

The present disclosure also provides a kit for detecting and/or screening for Weaver syndrome, or an increased likelihood of WVS, in a sample, comprising:

a) at least one detection agent for determining the methylation level of (i) at least one, optionally at least 3, at least 5, at least 10, at least 20, at least 30, at least 40, at least 50, or all CpG loci from Table 8, and/or (ii) at least one, optionally at least 2, at least 4, at least 6, or all the genes from Table 8; and

b) instructions for use.

In one embodiment, the kit further comprises bisulfite conversion reagents, methylation-dependent restriction enzymes, methylation-sensitive restriction enzymes, PCR reagents, probes and/or primers.

In yet another embodiment, the kit further comprises a computer-readable medium that causes a computer to compare methylation levels from a sample at the selected CpG loci to one or more control profiles and compute a correlation value between the sample and control profile.

### DRAWINGS

Embodiments are described below in relation to the drawings in which:

FIG. 1 identifies a large number of differentially methylated CGs between SS and control subjects. The volcano plot displays the relationship between difference in DNAm levels and significance between the two groups, using a scatter plot view. The y-axis is the negative log10 of P values (a higher value indicates greater significance) and the x-axis is the difference in DNAm between SS and Control (non-SS) subjects. Light grey horizontal line depicts significance level at p<0.05. Note that most of the significant CpGs are losing methylation in SS compared to non-SS. At 20% cut-off difference in DNA methylation, it is possible to clearly enrich for the highly significant CpGs with larger difference between SS and non-SS.

FIG. 2 shows a DNA methylation signature associated with NSD1 mutations. Unsupervised Hierarchical clustering of 72 samples based on the identified 7K differentially methylated CpG sites. All SS samples clustered together separate from non-SS control blood samples.

FIG. 3 depicts testing of the sensitivity and the specificity of the NSD1^{+/-} DNAm signature. Using 19 SS and 53 non-SS subjects, the median-methylation profiles of SS and non-SS controls were generated, respectively, on the 7K CG sites. The specificity of these profiles were tested on a test set of GEO blood samples (n=1056, light grey filled circles), all of which were more similar to the non-SS control profile (specificity 100%). The sensitivity was also estimated on a separate SS test set (n=19, represented as squares), all of which were more similar to the SS profile than to the non-SS control (100% sensitivity). Similarity was computed as the Pearson correlation to either the SS or the non-SS control DNAm profile. Out of 16 missense samples (represented as X), 9 classify with SS and 7 with controls. Also shown the classifications of the original 53 controls (represented as triangles) and 19 SS (represented as dark grey filled circles) used in the derivation of the SS and non-SS control signatures.

FIG. 4 shows that a NSD1^{+/-} DNAm signature can be identified in SS fibroblasts. Unsupervised Hierarchical clustering of 3 SS derived fibroblasts and 4 non-SS control derived fibroblasts clearly distinguish SS from control. In addition, tissue-specific DNAm is identified in fibroblast samples compared to controls.

FIG. 5 depicts a series of leave-one-out (LOO) cross-validations on the combined dataset of 47 Sotos cases (38 original SS cases+9 pathogenic missense cases) vs. 53 controls. At each LOO iteration, one sample was removed from the dataset for the subsequent validation step. The remaining samples were used to generate median DNA methylation profiles for the SS group and for the non-SS group. This classification was compared to the sample's true status (SS or non-SS). Based on these LOO results over all 47+53 samples, the specificity and sensitivity were estimated for various threshold combinations of delta beta (from 40% to 70%). In a second set of validations, an SS signature was created for different parameter combinations, and 1056 normal controls from the GEO collections were classified based on their closeness to SS or non-SS profiles. The number of samples correctly classified as “non-SS” contributed to the specificity estimate.

FIG. 6 shows unsupervised hierarchical clustering of the EZH2+/− DNA methylation signature. Differential DNA methylation analysis using t.test statistics and false discovery rate between 7 subjects with EZH2+/− single nucleotide variants (or missense mutations) and 52 control subjects identified 3216 CpG sites to be significantly different. Unsupervised hierarchical clustering using Euclidian distance metrics identified a subset of 6 subjects with EZH2+/− that clustered together separate of all control samples and one subject with EZH2+/− (Subject F0123) that clustered with the control samples.

FIG. 7 shows a principal component analysis revealing a clear separation between EZH2+/− and the controls.

FIG. 8 shows a principal component analysis for a minimal EZH2+/− signature.

FIG. 9 shows unsupervised hierarchical clustering of CpG sites showing the separation between Weaver syndrome and control samples.

FIG. 10 shows a gene cluster on chromosome 7 is regulated by EZH2. A chromosome-wide view of the DNA methylation difference between Weaver syndrome and control samples shows the mapping of the genomic region on the short arm of chromosome 7 overlapping the HOXA gene cluster.

### DESCRIPTION OF VARIOUS EMBODIMENTS

The inventors have conducted genome-wide DNA methylation (DNAm) profiling using blood from individuals with Sotos syndrome (SS), a disorder involving aberrant NSD1 function. Based on comparison of the DNA methylation profile from SS individuals to those of non-SS controls, the inventors have shown that DNA methylation profiles may be used in a test for early and accurate diagnosis of Sotos syndrome due to NSD1 pathogenic mutations. 7085 CpG loci (Table 1) were identified as showing a statistically significant (corrected p-value <0.05) difference in methylation levels between SS cases and non-SS controls.

The inventors have also conducted genome-wide DNA methylation (DNAm) profiling using blood from individuals with Weaver syndrome (WVS), a disorder involving aberrant EZH2 function. Based on comparison of the DNA methylation profile from WVS individuals to those of non-WVS controls, the inventors have shown that DNA methylation profiles may be used in a test for early and accurate diagnosis of Weaver syndrome due to EZH2 pathogenic mutations. 55 CpG Foci (Table 8) were identified as showing a difference in methylation levels between WVS cases and non-WVS controls.

I. Definitions

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used herein, the term “isolated” or “purified” when used in relation to a DNA molecule refers to a DNA molecule that is extracted and separated from one or more contaminants with which it naturally occurs.

As used herein, “methylation” refers specifically to DNA methylation, and more particularly to a modification in which a methyl group or hydroxymethyl group is added to the 5 position of a cytosine residue to form a 5-methyl cytosine (5-mCyt) or 5-hydroxymethylcytosines (5-hmC).

As used herein, “CpG locus” or “methylation locus” refers to an individual CpG dinucleotide sequence in genomic DNA which is capable of being methylated. Individual CpG loci may be identified by reference to an Illumina CpG locus (Illumina ID #) which is defined by a chromosome number, genomic coordinate, genome build (NCBI hg 19, genome build 37), and +/− strand designation to unambiguously define each CpG locus. The genomic information is publically available through the UCSC genome browser at https://genome.ucsc.edu/.

The term “methylation level” refers to a measure of the amount of methylation at a target site (for example, a CpG locus) within a DNA molecule in a sample. For example, the level of methylation can be measured for one or more CpG dinucleotides, or for a region of DNA. If the methylation level of a target site within a sample is higher than a reference level, the sample is considered to have increased methylation relative to the reference at the target site. Conversely, if the methylation level of a target site within a sample is lower than the reference level, the sample is considered to have a decreased methylation level relative to the reference at the target site. The target site may be an individual CpG locus or a region of DNA comprising multiple CpG loci, for example, a gene promoter. Methylation levels of a target site may be measured by methods known in the art, for example, as a “βvalue” or “beta value”, which is calculated as:

β value=intensity of the methylated target (M)/(intensity of the unmethylated target (U)+intensity of the methylated target (M)+100)

A β value of zero indicates no methylation and a value of one indicates 100% methylation.

As used herein, the term “methylation status” refers to whether a specified target DNA site is methylated or not methylated. The target site may be an individual CpG locus, a region of DNA comprising multiple CpG loci, for example, a gene promoter or a gene. For example, a target site may have a methylation status of “methylated” or “hypermethylated” if the target has significantly higher methylation beta value in a SS (or WVS) specific control profile compared to a non-SS (or non-WVS) control profile. Conversely, a target site may have a methylation status of “not methylated” or “hypomethylated” if the target has significantly lower methylation beta value in a SS (or WVS) specific control profile compared to a non-SS (or non-WVS) control profile.

As used herein, the term “delta beta” or “delta β” refers to the difference between the β value of a methylation target in two different samples, for example, the β value of a methylation target in a SS (or WVS) specific control profile and the β value of the same methylation target in a non-SS (or non-WVS) control profile.

As used herein the term “gene” refers to a genomic DNA sequence that comprises a coding sequence associated with the production of a polypeptide or polynucleotide product (e.g., rRNA, tRNA). The methylation level of a gene as used herein, encompasses the methylation level of sequences which are known or predicted to affect expression of the gene, including the promoter, enhancer, and transcription factor binding sites. As used herein, the term “enhancer” refers to a cis-acting region of DNA that is located up to 1 Mbp (upstream or downstream) of a gene.

As used herein, the term “sample methylation profile” or “sample profile” refers to the methylation levels at one or more target sequences in a subject's genomic DNA. The target sequence may be an individual CpG locus or a region of DNA comprising multiple CpG loci, for example, a gene promoter or CpG island. The methylation profile of a sample tested according the methods disclosed herein is referred to as a sample profile.

In some embodiments, the sample methylation profile is compared to one or more control profiles. The control profile may be a reference value and/or may be derived from one or more samples, optionally from historical methylation data for a patient or pool of patients who are known to have, or not have, Sotos syndrome or Weaver syndrome. In such cases, the historical methylation data can be a value that is continually updated as further samples are collected and individuals are identified as SS or not-SS or WVS or not-WVS. It will be understood that the control profile represents an average of the methylation levels for selected CpG loci as described herein. Average methylation values may, for example, be the mean values or median values.

For example, a “SS specific control profile” or “SS control profile” may be generated by measuring the methylation levels at specified target sequences in genomic DNA from an individual subject, or population of subjects, who are known to have SS and an NSD1 pathogenic mutation. Similarly, a “non-SS control profile” may be generated by measuring the methylation levels at specified target sequences in genomic DNA from an individual subject or population of subjects who are known to not have SS. In certain embodiments, the tissue source from which the sample profile and control profile are derived is matched, so that they are both derived from the same or similar tissue.

Further, a “WVS specific control profile” or “WVS control profile” may be generated by measuring the methylation levels at specified target sequences in genomic DNA from an individual subject, or population of subjects, who are known to have WVS and an EZH2 pathogenic mutation. Similarly, a “non-WVS control profile” may be generated by measuring the methylation levels at specified target sequences in genomic DNA from an individual subject or population of subjects who are known to not have WVS. In certain embodiments, the tissue source from which the sample profile and control profile are derived is matched, so that they are both derived from the same or similar tissue

As used herein, the phrase “detecting and/or screening” for a condition refers to a method or process of determining if a subject has or does not have said condition. Where the condition is a likelihood or risk for a disease or disorder, the phrase “detecting and/or screening” will be understood to refer to a method or process of determining if a subject is at an increased or decreased likelihood for the disease or disorder.

As used herein, the term “sensitivity” refers to the ability of the test to correctly identify those patients with the disease or disorder, such that a 100% sensitivity indicates a test that correctly identifies all patients with the disease or disorder. Sensitivity is calculated as:

Sensitivity=(True Positives)/(True Positives+False Negatives).

A high sensitivity as used herein refers to a sensitivity of greater than 50%.

As used herein, the term “specificity” refers the ability of a test to correctly identify those patients without the disease or disorder, such that a 100% specificity indicates a test that correctly identifies all patients without the disease or disorder. Specificity is calculated as:

Specificity=(True Negatives)/(True Negatives+False Positives).

A high specificity as used herein refers to a specificity of greater than 50%.

As used herein, the term “CpG” or “CG” site refers to cytosine and guanosine residues located sequentially (5′->3′) in a polynucleotide DNA sequence. The term “CpG island” refers to a region of genomic DNA characterized by a high frequency of CpG sites, for example, a CpG island may be characterized by CpG dinucleotide content of at least 60% over the length of the island. As used herein the term “CpG island shore” refers to a region of DNA occurring within 2 kbp (upstream or downstream) of a CpG island. As used herein the term “body” (in reference to a gene) refers to the genomic region covering the entire gene from the transcription start site to the end of the transcript. As used herein the term “distance from TSS” refers to the genomic difference in base pairs between specific CpG locus and the nearest transcription start site.

As used herein, a first CpG locus is “associated” with a second CpG locus, if the methylation status at the first locus is reasonably predictive of the methylation status of the second locus and vice versa. CpG loci may be considered “associated”, for example, if they occur within the same CpG island, CpG island shore, gene promoter or gene enhancer region. CpG loci may also be considered “associated” by virtue of their genomic proximity, for example, CpG loci residing within 300 nucleotides, optionally within 150 nucleotides, of each other may be considered associated.

As used herein, the term “treating DNA from the sample with bisulfite” refers to treatment of DNA with a reagent comprising bisulfite, disulfite, hydrogen sulfite or combinations thereof, for a time and under conditions sufficient to convert unmethylated DNA cytosine residues to uracil, thereby facilitating the identification of methylated and unmethylated CpG dinucleotide sequences. Bisulfite modifications to DNA may be detected according to methods known in the art, for example, using sequencing or detection probes which are capable of discerning the presence of a cytosine or uracil residue at the CpG site.

The term “subject” as used herein refers to a human subject and includes, for example, a fetus.

The terms “complementary” or “complementarity” are used in reference to a first polynucleotide (which may be an oligonucleotide) which is in “antiparallel association” with a second polynucleotide (which also may be an oligonucleotide). As used herein, the term “antiparallel association” refers to the alignment of two polynucleotides such that individual nucleotides or bases of the two associated polynucleotides are paired substantially in accordance with Watson-Crick base-pairing rules. Complementarity may be “partial,” in which only some of the polynucleotides' bases are matched according to the base pairing rules. Or, there may be “complete” or “total” complementarity between the polynucleotides. Those skilled in the art of nucleic acid technology can determine duplex stability empirically by considering a number of variables, including, for example, the length of the first polynucleotide, which may be an oligonucleotide, the base composition and sequence of the first polynucleotide, and the ionic strength and incidence of mismatched base pairs.

The term “hybridize” refers to the sequence specific non-covalent binding interaction with a complementary nucleic acid. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C. for 15 minutes, followed by a wash of 2.0×SSC at 50° C. for 15 minutes may be employed.

The stringency may be selected based on the conditions used in the wash step. For example, the salt concentration in the wash step can be selected from a high stringency of about 0.2×SSC at 50° C. for 15 minutes. In addition, the temperature in the wash step can be at high stringency conditions, at about 65° C. for 15 minutes.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log10 [Na+])+0.41(%(G+C)−600/I), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% sequence identity, the final wash temperature will be reduced by about 5° C. Based on these considerations those skilled in the art will be able to readily select appropriate hybridization conditions. In an embodiment, stringent hybridization conditions are selected. By way of example the following conditions may be employed to achieve stringent hybridization: hybridization at 5× sodium chloride/sodium citrate (SSC)/5× Denhardt's solution/1.0% SDS at Tm −5° C. based on the above equation, followed by a wash of 0.2×SSC/0.1% SDS at 60° C. for 15 minutes. Moderately stringent hybridization conditions include a washing step in 3×SSC at 42° C. for 15 minutes. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 2000, Third Edition.

The term “oligonucleotide” as used herein refers to a nucleic acid substantially free of cellular material or culture medium when produced by recombinant DNA techniques, or chemical precursors, or other chemicals when chemically synthesized. The term “nucleic acid” and/or “oligonucleotide” as used herein refers to a sequence of nucleotide or nucleoside monomers consisting of naturally occurring bases, sugars, and intersugar (backbone) linkages, and is intended to include DNA and RNA which can be either double stranded or single stranded, represent the sense or antisense strand. The term also includes modified or substituted oligomers comprising non-naturally occurring monomers or portions thereof.

As used herein, the term “amplify”, “amplifying” or “amplification” of DNA refers to the process of generating at least one copy of a DNA molecule or portion thereof. Methods of amplification of DNA are well known in the art, including but not limited to polymerase chain reaction (PCR), ligase chain reaction (LCR), self-sustained sequence replication (3SR), nucleic acid sequence based amplification (NASBA), strand displacement amplification (SDA), multiple displacement amplification (MDA) and rolling circle amplification (RCA).

II. Methods

As set out in Table 1, the instant disclosure identifies 7085 distinct CpG loci (the “7K” CG sites), each of which show a statistically significant (corrected p-value <0.05) difference in methylation levels between individuals with SS and non-SS controls over the tested population. As described in the Examples, the methylation levels of the disclosed loci, or a subset thereof, may be used in diagnostic testing for SS, with up to 100% sensitivity and specificity. It will be understood that the sensitivity and specificity of the methods described will tend to increase with the number of CpG loci or sites selected for testing (i.e. the size of the signature), to a maximal sensitivity/specificity of 100%. However, signatures utilizing fewer CpG loci, for example as few as 7 (Example 4), are described herein which retain greater than 50% sensitivity and specificity and are useful for assessing likelihood of Sotos syndrome.

As set out in Table 8, the instant disclosure also identifies 55 distinct CpG loci, each of which show a difference in methylation levels between individuals with WVS and non-WVS controls over the tested population. As described in the Examples, the methylation levels of the disclosed loci, or a subset thereof, may be used in diagnostic testing for WVS, with up to 100% sensitivity and specificity. It will be understood that the sensitivity and specificity of the methods described will tend to increase with the number of CpG loci or sites selected for testing (Le. the size of the signature), to a maximal sensitivity/specificity of 100%. However, signatures utilizing fewer CpG loci, are described herein which retain greater than 50% sensitivity and specificity and are useful for assessing likelihood of Weaver syndrome.

Useful methylation signatures according to the described methods are not intended to be limited to the 7K CG sites of Table 1 and the 55 CG sites of Table 8, but are intended to include associated CpG loci, and associated gene and non-gene regions. DNA methylation at a single CpG locus can predict DNA methylation of multiple other loci residing in near genomic proximity or overlapping CpG islands. Accordingly, “associated” loci and regions are loci and regions, the methylation levels or status of which may be reasonably predicted by the methylation levels or status of one or more of the CpG loci of Table 1 or Table 8. CpG loci may be considered “associated”, for example, if they occur within the same CpG island, CpG island shore, gene promoter or gene enhancer region. CpG loci may also be considered “associated” by virtue of their proximity, for example, CpG loci residing within 300 nucleotides, optionally within 150 nucleotides, of each other may be considered associated.

Accordingly, an aspect of the disclosure provides a method of detecting and/or screening for Sotos syndrome (SS), or an increased likelihood of SS, in a human subject, comprising determining a sample methylation profile from a sample of DNA from said subject, said sample profile comprising the methylation level of at least one, optionally at least 7, at least 10, at least 17, at least 25, at least 41, at least 57, at least 87, at least 124, at least 156, at least 220, at least 287, at least 399, at least 549, at least 726, or all CpG loci selected from (i) Table 1 and/or (ii) associated CpG loci residing within 300 nucleotides, optionally within 150 nucleotides, of the CpG loci of (i).

Another aspect of the disclosure provides a method of detecting and/or screening for Weaver syndrome (WVS), or an increased likelihood of WVS, in a human subject, comprising determining a sample methylation profile from a sample of DNA from said subject, said sample profile comprising the methylation level least one, optionally at least 3, at least 5, at least 10, at least 20, at least 30, at least 40, at least 50, or all CpG loci from (i) Table 8 and/or (ii) associated CpG loci residing within 300 nucleotides, optionally within 150 nucleotides, of the CpG loci of (i).

Methods of DNA methylation profiling of target genomic regions are generally known in the art (Stevens et al 2013, Harris et al 2010 and Hirst 2013).

For example, a non-limiting list of exemplary methods that may be used to determine methylation levels at a specified target sequence of DNA include: bisulfite sequencing, pyrosequencing, methylation-sensitive single-strand conformation analysis (MS-SSCA), high resolution melting analysis (HRM), methylation-sensitive single nucleotide primer extension (MS-SnuPE), base-specific cleavage/MALDI-TOF, methylation-specific PCR (MSP), methylation-sensitive restriction enzyme-based methods and/or microarray-based methods.

In an embodiment, methylation levels are measured using an agent that provides for determination of a CpG methylation status of at least one, optionally all, of the selected CpG loci, wherein the agent comprises an oligonucleotide-immobilized substrate comprising a plurality of capture probes, each capture probe comprising a pair of capture oligonucleotides, wherein the capture oligonucleotide pairs comprise (a) an oligonucleotide comprising nucleotide sequence complementary to or identical to a nucleotide sequence of genomic DNA comprising a selected CpG loci, and (b) an oligonucleotide comprising nucleotide sequence complementary to or identical to a nucleotide sequence of genomic DNA comprising the same selected CpG loci of (a), in which the cytosine residue of the CpG loci is replaced with a thymine residue. A non-limiting example of such an agent includes a “microarray”, comprising an ordered set of probes fixed to a solid surface that permits analysis such as methylation analysis of a plurality of genomic targets sequences.

According to the methods described herein, similarity of the

DNA methylation profile from a sample to one or more control profiles, may be used to identify individuals having Sotos syndrome, or an increased likelihood of having Sotos syndrome. For example, in an embodiment, the method comprises determining the level of similarity of a sample profile to one or more control profiles, wherein (i) a high level of similarity of the sample profile to an SS specific profile; (ii) a low level of similarity to a non-SS control profile; and/or (iii) a higher level of similarity to a SS specific profile than to a non-SS control profile indicates the presence of, or an increased likelihood of, SS.

Similarity of the DNA methylation profile from a sample to one or more control profiles, may be used to identify individuals having Weaver syndrome, or an increased likelihood of having Weaver syndrome. For example, in an embodiment, the method comprises determining the level of similarity of a sample profile to one or more control profiles, wherein (i) a high level of similarity of the sample profile to a WVS specific profile; (ii) a low level of similarity to a non-WVS control profile; and/or (iii) a higher level of similarity to a WVS specific profile than to a non-WVS control profile indicates the presence of, or an increased likelihood of, WVS.

It will be appreciated that the control profile may be a reference value, or derived from one or more samples, optionally from historical methylation data for a patient or pool of patients. The control profile may be a reference value and/or may be derived from one or more samples, optionally from historical methylation data for a patient or pool of patients who are known to have, or not have, Sotos syndrome or Weaver syndrome. In such cases, the historical methylation data can be a value that is continually updated as further samples are collected and individuals are identified as SS or not-SS or WVS or not-WVS. For example, the control database may be stored on an online database, which is continually updated with methylation data from diagnosed SS and non-SS patients and/or diagnosed WVS and non-WVS patients. It will be understood that the control profile represents an average of the methylation levels for selected CpG loci as described herein.

In an embodiment, the “SS specific control profile” is generated by measuring the methylation levels at specified target sequences in genomic DNA from an individual subject, or population of subjects, who are known to have SS. Similarly, in an embodiment, the “non-SS control profile” is generated by measuring the methylation levels at specified target sequences in genomic DNA from an individual subject, or population of subjects, who are known to not have SS. In certain embodiments, the tissue source from which the sample profile and control profile are derived is matched, so that they are both derived from the same or similar tissue. In other embodiments, the sample profile and control profile are derived from different tissues. In certain other embodiments, the SS specific control profile and the non-SS control profile are derived from historical data and can indicate similarity of a sample to either the SS or non-SS profiles.

In another embodiment, the “WVS specific control profile” is generated by measuring the methylation levels at specified target sequences in genomic DNA from an individual subject, or population of subjects, who are known to have WVS. Similarly, in an embodiment, the “non-WVS control profile” is generated by measuring the methylation levels at specified target sequences in genomic DNA from an individual subject, or population of subjects, who are known to not have WVS. In certain embodiments, the tissue source from which the sample profile and control profile are derived is matched, so that they are both derived from the same or similar tissue. In other embodiments, the sample profile and control profile are derived from different tissues. In certain other embodiments, the WVS specific control profile and the non-WVS control profile are derived from historical data and can indicate similarity of a sample to either the WVS or non-WVS profiles.

Methods of determining the similarity between methylation profiles are well known in the art. Methods of determining similarity may in some embodiments provide a non-quantitative measure of similarity, for example, using visual clustering. In another embodiment, similarity may be determined using methods which provide a quantitative measure of similarity.

For example, in an embodiment, similarity may be measured using hierarchical clustering, optionally using Manhattan distance. For example, unsupervised hierarchical clustering of the said sample with the SS specific control profile indicate similarity to an SS and if the said sample cluster with the non-SS control profile, this will indicate similarity to non-SS control profile.

The Manhattan distance function computes the distance that would be traveled to get from one data point to the other if a grid-like path is followed. The Manhattan distance between two items is the sum of the differences of their corresponding components.

The formula for this distance between a point X=(X1, X2, etc.) and a point Y=(Y1, Y2, etc.) is:

$$d = {\sum }_{i = 1}^{n} \vert {x}_{i} - {y}_{i} \vert$$

Where n is the number of variables, and Xi and Yi are the values of the variable, at points X and Y respectively.

In another embodiment, similarity may be measured by computing a “correlation coefficient”, which is a measure of the interdependence of random variables that ranges in value from −1 to +1, indicating perfect negative correlation at −1, absence of correlation at zero, and perfect positive correlation at +1. In an embodiment, the correlation coefficient may be a linear correlation coefficient, for example, a Pearson product-moment correlation coefficient.

A Pearson correlation coefficient (r) is calculated using the following formula:

$$r = \frac{{\sum }_{i} ( {x}_{i} - \overline{x} ) ( {y}_{i} - \overline{y} )}{\sqrt{{\sum }_{i} {( {x}_{i} - \overline{x} )}^{2}} \sqrt{{\sum }_{i} {( {y}_{i} - \overline{y} )}^{2}}}$$

In one embodiment, x and y are the beta values for various CpG loci in a sample profile and a control profile, respectively.

In an embodiment, a correlation coefficient calculated between the sample profile and the control profile indicates a high level of similarity to the control profile when the correlation coefficient has an absolute value between 0.5 to 1, optionally between 0.75 to 1, and a low level of similarity to the control profile when the correlation coefficient has an absolute value between 0 to 0.5, optionally between 0 to 0.25.

It will be appreciated that any “correlation value” which provides a quantitative scaling measure of similarity between methylation profiles may be used to measure similarity.

A sample profile may be identified as belonging to an individual with SS, or an increased likelihood of SS, where the sample profile has high similarity to the SS profile, low similarity to the non-SS profile, or higher similarity to the SS profile than to the non-SS profile. Conversely, a sample profile may be identified as belonging to an individual without SS, or an decreased likelihood of SS, where the sample profile has high similarity to the non-SS profile, low similarity to the SS profile, or higher similarity to the non-SS profile than to the SS profile.

For example, in an embodiment, a sample profile may be identified as belonging to an individual with SS, or an increased likelihood of SS, based on calculation of a Sotos Syndrome Score, which generally is defined by the following formula:

SS score(B)=r(B, SS profile)−r(B, control profile)

where r is the Pearson correlation coefficient, and B is a vector of DNA methylation levels across the selected CpG loci.

A sample profile with a positive Sotos Syndrome Score is more similar to the SS specific profile across the selected CpG loci, and is therefore classified as “SS”; whereas a sample with a negative Sotos Syndrome Score is more similar to the non-SS profile across the selected CpG loci, and is classified as “not SS”.

A sample profile may be identified as belonging to an individual with WVS, or an increased likelihood of WVS, where the sample profile has high similarity to the WVS profile, low similarity to the non-WVS profile, or higher similarity to the WVS profile than to the non-WVS profile. Conversely, a sample profile may be identified as belonging to an individual without WVS, or an decreased likelihood of WVS, where the sample profile has high similarity to the non-WVS profile, low similarity to the WVS profile, or higher similarity to the non-WVS profile than to the WVS profile.

For example, in an embodiment, a sample profile may be identified as belonging to an individual with WVS, or an increased likelihood of WVS, based on calculation of a Weaver Syndrome Score, which generally is defined by the following formula:

WVS score(B)=r(B, WVS profile)−r(B, control profile)

where r is the Pearson correlation coefficient, and B is a vector of DNA methylation levels across the selected CpG loci.

A sample profile with a positive Weaver Syndrome Score is more similar to the WVS specific profile across the selected CpG loci, and is therefore classified as “WVS”; whereas a sample with a negative Weaver Syndrome Score is more similar to the non-WVS profile across the selected CpG loci, and is classified as “not WVS”.

As used herein the term “sample” refers to a biological sample comprising genomic DNA from a human subject. The sample may, for example, comprise blood, fibroblast tissue, buccal tissue, and/or amniotic fluid.

Median methylation levels for SS and non-SS cases reported in Table 1 were identified using whole blood samples. However, the distinct methylation profiles observed in SS cases was also present in fibroblasts and amniocytes. For example, as shown in Example 3, a large subset of the CpG sites from the SS specific DNAm signature shared similar DNA methylation profiles in both blood and fibroblast tissues suggesting that the SS-specific profiles will be present in multiple tissues of individuals with Sotos syndrome and NSD1 mutations.

Another aspect provides, a method of detecting and/or screening for Sotos syndrome (SS), or an increased likelihood of SS, in a human subject, comprising determining the methylation level of at least one, optionally at least 6, at least 12, at least 17, at least 18, at least 24, at least 33, at least 49, at least 67, at least 72, at least 101, at least 127, at least 164, at least 212, at least 275, or all the genes from Table 1 in a sample comprising genomic DNA from the subject, wherein a difference in methylation levels of genes selected from Table 1, relative to a non-SS control profile is indicative of SS, or an increased likelihood of SS, in the subject.

Another aspect provides, a method of detecting and/or screening for Weaver syndrome (WVS), or an increased likelihood of WVS, in a human subject, comprising determining the methylation level of at least one, optionally at least 2, at least 4, at least 6, or all the genes from Table 8 in a sample comprising genomic DNA from the subject, wherein a difference in methylation levels of genes selected from Table 8, relative to a non-WVS control profile is indicative of WVS, or an increased likelihood of WVS, in the subject.

Diagnostic certainty for Sotos syndrome aids in medical management by enabling targeted medical testing and intervention. Targeted testing includes, but is not limited to, echocardiogram to screen for congenital heart defects, renal ultrasound to screen for congenital renal anomalies, audiology and ophthalmology assessment to screen for hearing and vision problems and active clinical monitoring to screen for scoliosis. In light of the increased incidence of non-febrile seizures, thorough electrophysiologic investigation of any suspected seizure activity would be indicated. Knowledge of the increased risk for tumors directs more aggressive investigation of common symptoms to enable early diagnosis and to maximize the opportunity for early intervention. In addition, neuropsychological assessment to screen for developmental/learning disabilities (individuals with Sotos syndrome have mild to moderate intellectual disability) and behavioural problems (e.g. aggression) provides the opportunity for early identification and optimal intervention. There is also an increased risk for other neuropsychological issues including ADHD and ASD, for which early diagnosis provides the opportunity for early intervention and improved outcomes. In summary, early identification of the above medical and cognitive issues provides the opportunity for an enhanced quality of life for individuals with Sotos syndrome.

Molecular confirmation of a diagnosis of Weaver syndrome is of value as it aids in medical management by enabling targeted medical testing and intervention and eliminating the need for screening for symptoms of other overgrowth syndromes. Targeted intervention for patients with Weaver syndrome includes, but is not limited to, active clinical monitoring to screen for scoliosis and follow-up of camptodactyly to monitor the need for surgical intervention. In addition, knowledge of the increased risk for tumors directs more aggressive investigation of common symptoms to enable early diagnosis and to maximize the opportunity for early intervention. In addition, neuropsychological assessment to screen for developmental/learning disabilities (individuals with Weaver syndrome typically have an intellectual disability) provides the opportunity for early identification and optimal intervention. Individuals with Weaver syndrome also present with hypotonia and ligamentous laxity and may benefit from physiotherapy. In summary, early identification of the above medical and cognitive issues provides the opportunity for an enhanced quality of life for individuals with Weaver syndrome.

Accordingly, an aspect provides a method of assigning a course of management for an individual with Sotos syndrome (SS), or an increased likelihood of SS, comprising:

a) identifying an individual with SS or an increased likelihood of SS, according the methods described herein; and

b) assigning a course of management for SS and/or symptoms of a SS, comprising i) testing for at least one medical condition associated with SS and ii) applying an appropriate medical intervention based on the results of the testing.

In one embodiment, the medical condition is selected from congenital heart defects, congenital renal abnormalities, hearing problems, vision problems, scoliosis, seizure activity, developmental/learning disabilities, behavioural problems and neuropsychological issues.

Another aspect provides a method of assigning a course of management for an individual with Weaver syndrome (WVS), or an increased likelihood of WVS, comprising:

a) identifying an individual with WVS or an increased likelihood of WVS, according the methods described herein; and

b) assigning a course of management for WVS and/or symptoms of a WVS, comprising i) testing for at least one medical condition associated with WVS and ii) applying an appropriate medical intervention based on the results of the testing.

In one embodiment, the medical condition is selected from scoliosis, camptodactyly, tumor growth, developmental/learning disabilities, intellectual disabilities, hypotonia and ligamentous laxity.

As used herein, the term “a course of management” refers to any testing, treatment, medical intervention and/or therapy applied to an individual with SS or WVS and/or symptoms of SS or WVS. Medical interventions include, but are not limited to, pharmaceutical treatments, surgical procedures, utilization of medical devices such as hearing aids or glasses, physical or occupational therapy and behavioral or cognitive therapy.

III. Kits

Another aspect provides a kit for detecting and/or screening for Sotos syndrome, or an increased likelihood of SS, in a sample, comprising:

a) at least one detection agent for determining the methylation level of:
i) at least one, optionally at least 7, at least 10, at least 17, at least 25, at least 41, at least 57, at least 87, at least 124, at least 156, at least 220, at least 287, at least 399, at least 549, at least 726, or all CpG loci selected from Table 1 or CpG loci residing within 300 nucleotides, optionally within 150 nucleotides, of the CpG loci in Table 1, and/or

ii) at least one, optionally at least 6, at least 12, at least 17, at least 18, at least 24, at least 33, at least 49, at least 67, at least 72, at least 101, at least 127, at least 164, at least 212, at least 275, or all the genes from Table 1; and

b) instructions for use.

Another aspect provides a kit for detecting and/or screening for Weaver syndrome, or an increased likelihood of WVS, in a sample, comprising:

a) at least one detection agent for determining the methylation level of:
i) at least one, optionally at least 3, at least 5, at least 10, at least 20, at least 30, at least 40, at least 50, or all CpG loci from Table 8 or CpG loci residing within 300 nucleotides, optionally within 150 nucleotides, of the CpG loci in Table 8, and/or
ii) at least one, optionally at least 2, at least 4, at least 6, or all the genes from Table 8; and

b) instructions for use.

In an embodiment, the kit further comprises bisulfite conversion reagents, methylation-dependent restriction enzymes, methylation-sensitive restriction enzymes, PCR reagents, probes and/or primers.

In an embodiment, the kit further comprises a computer-readable medium that causes a computer to compare methylation levels from a sample at the selected CpG loci or genes to one or more control profiles and compute a correlation value between the sample and control profile.

Other features and advantages of the disclosure will become apparent from the following detailed description. It should be understood, however, that the description and the specific examples while indicating preferred embodiments are given by way of illustration only, since various changes and modifications within the spirit and scope of the disclosure will become apparent to those skilled in the art from this description of various embodiments.

The following non-limiting examples are illustrative of the present disclosure:

### EXAMPLES

### Example 1

### Identification of DNAm Signature in SS Patients with NSD1 Pathogenic Mutations

### Materials and Methods

### Sample Collection

### Discovery Cohort

Individuals with a clinical diagnosis of Sotos syndrome and a pathogenic NSD1 mutation were recruited through an REB approved research study at the Hospital for Sick Children in Toronto, Ontario. A clinical diagnosis of Sotos syndrome was established based on the following criteria: height +/− weight greater than 2 SD above the mean, macrocephaly (OFC >2SD), developmental delay and characteristic facial gestalt. Informed consent was obtained from parents of all participants and assent was obtained from participants, as appropriate based on age. DNA from blood samples was extracted by standard methods. In all, 19 individuals, including one familial case with three affected individuals (father and two children), were included. All of these patients had a pathogenic NSD1 mutation, including a whole gene deletion or truncating mutation. In addition, six individuals with an overgrowth phenotype (some with a suspected clinical diagnosis of Sotos syndrome and some without the Sotos' gestalt) who were identified to have a missense mutation (single nucleotide substitution) that was classified as a variant of unknown significance, were also enrolled in the study. All patients with missense mutations were examined in person, or via medical records and photographs, independently by two reviewers with extensive clinical experience with Sotos syndrome (RW), who were blinded to the methylation results. Patients were classified into one of three phenotypic categories: (1) “typical Sotos syndrome”, (2) “possible Sotos syndrome”, and (3) “unlikely Sotos syndrome”. There was 100% concordance (6/6) between the clinical classification of both reviewers and the methylation status. Specifically, four patients were categorized as “typical Sotos syndrome”, all of whom had the DNAm signature and two patients were categorized as “unlikely Sotos syndrome”, neither of whom had the signature. The specific NSD1 mutations and clinical features of the patient cohort are summarized in Table 2.

### Validation Cohort

An additional 19 patients with a clinical diagnosis of Sotos syndrome and confirmed pathogenic NSD1 mutation (whole gene deletion or truncating mutations) and 10 patients with missense mutations were obtained from the University of Hong Kong. All patients with missense mutations for whom photographs and medical records were available (6/10) were assessed independently by two experienced clinicians who were blinded to the methylation results. Patients were assigned to one of the three phenotypic categories stipulated above. The clinicians' assessments were concordant for 5/6 patients. Specifically, two of the patients were categorized by both clinicians as “unlikely Sotos”; these patients did not have the methylation signature. Two of the patients were categorized by both clinicians as “typical Sotos”; these patients did have the methylation signature. One patient with the methylation signature was categorized by both clinicians as “possible Sotos”. In a single case, the clinicians' opinions were discordant, with one categorizing the patient as “possible Sotos syndrome” and the other as “unlikely Sotos syndrome”. Of note, this patient had the most limited clinical information available for review.

### Control Cohort

Genomic DNA from 53 consented control blood samples recruited at The Hospital for Sick Children following was used. DNA methylation data from an additional 1056 control blood samples were downloaded from the gene expression omnibus (GEO) public database (http://www.ncbi.nlm.nih.gov/geo/). This database is a repository of high throughput gene expression data and hybridization arrays, chips and microarrays.

### DNA Methylation Analysis

1 microgram of genomic DNA was subjected to bisulfite conversion using the Qiagen EZ DNA Methylation™ kit (Qiagen), according to the manufacturer's protocols. Modified genomic DNA was processed as described in the Infinium® Assay Methylation Protocol Guide Rev. C (November 2010) and analyzed on Infinium® HumanMethylation450 BeadChips (Illumine). Probes that had missing values, had detection p-value <0.01 and non-specific probes as well as known bound targets with known sequence variants [Chen et al, 2013a] were removed. The final set contained 424,586 probes.

### Differential DNA Methylation Analysis

To identify the differentially methylated CpG sites, the DNAm distributions in Sotos cases were compared against controls at each CpG site. To account for the influence of the family relationships among three of the Sotos subjects, three separate testing trials were formed, each time combining 16 non-familial Sotos cases with only one family member. The resulting set of 17 Sotos subjects was compared to 53 controls for each of the 424,586 available CpG sites, using a non-parametric Mann-Whitney U test. A stringent Bonferroni correction for multiple testing was applied to the results in each trial. In order to ensure robust results, only the CpG sites that were significant at the confidence level α=0.05 in all three trials were retained, i.e. with any choice of the family representative among the Sotos subjects. As many as 28458 CpG sites satisfied this criterion. Finally, an additional effect-size criterion requiring at least 20% difference in DNA methylation between the Sotos and the control groups in each of the three trials was applied. This filter reduced the NSD1 signature to the robust set of 7085 CpG sites, which were selected for further characterization, and for specificity and sensitivity testing in independent control and SS case cohorts.

### Sotos Syndrome Score and Classification Model

A simple classification model based on the NSD/^{+/-} DNAm signature in blood was developed. At each of the 7K CGs, a median DNAm level was computed across all 19 SS subjects in the original Discovery cohort. This resulted in a reference profile of the NSD1^{+/-} Sotos DNAm level over the 7K CGs, which was robust to outliers. Similarly, a robust median-DNAm reference profile for the 53 healthy control subjects was created. The classification of each new DNAm sample was based on extracting a vector B_{7K} of its DNAm values in the 7K CGs, and comparing B_{7K} to the two reference profiles computed above. A Sotos Syndrome Score was defined as:

SS score(B)=r(B_{7K}, SS profile)−r(B_{7K}, control profile)

where r is the Pearson correlation coefficient. A simple classification model was developed based on scoring each new DNAm sample using SSS: asample with a positive Sotos Syndrome Score is more similar to the SS reference profile based on the 7K CGs, and is therefore classified as “SS”;

whereas a sample with a negative Sotos Syndrome Score is more similar to the normal reference profile, and is classified as “not SS”.

### Results

DNA methylation was compared in peripheral blood from SS cases (NSD1^{+/-}; n=19) to controls (n=53). Initially known pathogenic mutations including NSD1 whole gene deletions and truncating mutations (NSD1^{+/-}) were used (Table 2) as well as one familial case of Sotos syndrome.

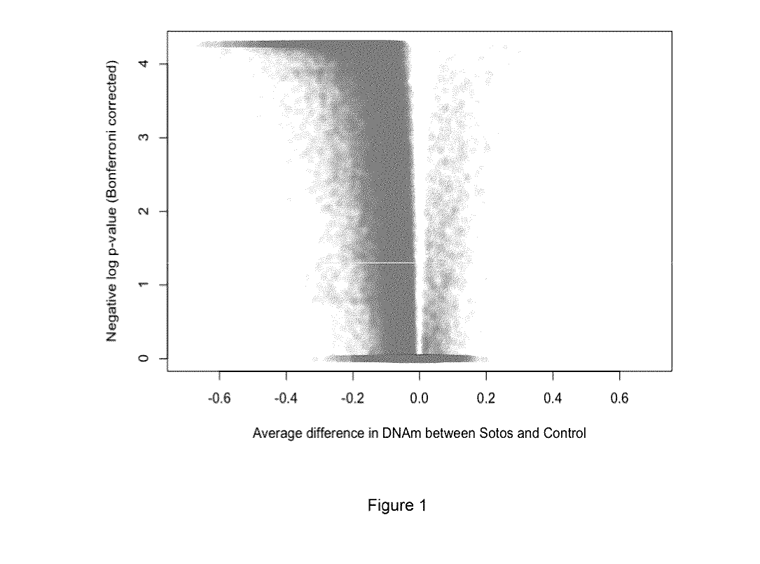

Genomic DNA obtained from whole blood was bisulfite treated and assessed using the Illumina Human Infinium® Methylation450 BeadChip. After filtering for non-specific probes as previously described [Chen et al, 2013b], methylation was quantified (beta values; range: 0-1) at 424,586 CpG sites. The significance of differential DNA methylation between the Sotos and control samples was assessed at each CpG using a non-parametric Mann-Whitney U test with a stringent Bonferroni correction for multiple testing. A largely specific signature for NSDI loss of function mutation was identified by selecting the set of probes that achieved statistical significance (corrected p<0.05) and had at least 20% difference in DNA methylation levels between the Sotos and non-Sotos control groups (FIG. 1). 7085 CpG sites (or 7K CGs) that were distributed across the genome were identified; 7038 CpG sites (99.3%) demonstrated loss of DNA methylation (Table 1) whereas only 47 CpG sites (0.6%) exhibited gain of methylation in SS compared to controls. Using unsupervised hierarchical clustering of the 7K CGs, all samples with NSD1^{+/-} faithfully clustered as a distinct group separate from controls (FIG. 1).

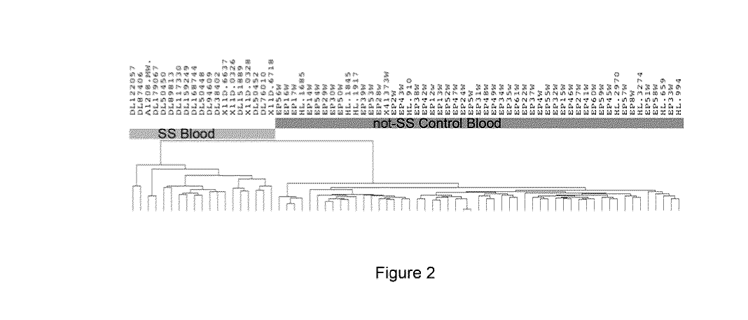

The NSD1^{+/-} DNAm signature was used to develop a predictive model, which classifies each new sample based on its DNAm profile as either SS or not-SS, using the Sotos Syndrome score (SS score; see above). The specificity of the SS score was tested using a large independent test set of normal DNAm blood samples (n=1056 subjects) extracted from the GEO database (www.ncbi.nlm.nih.gov/geo/). Each of the 1056 samples from the GEO collection received a negative SS score and were classified as “not SS” (FIG. 2), suggesting a 100% specificity of the classification model. The size and the diversity of data sources in the GEO test dataset suggest that new samples from normal patients are highly likely to be classified correctly as “not Sotos” by using the NSD1^{+/-} DNAm signature.

The sensitivity of the SS score was also tested as a predictor of SS using a replication cohort of cases with whole gene deletions and truncating mutations (n=19) from Hong Kong (Table 3).

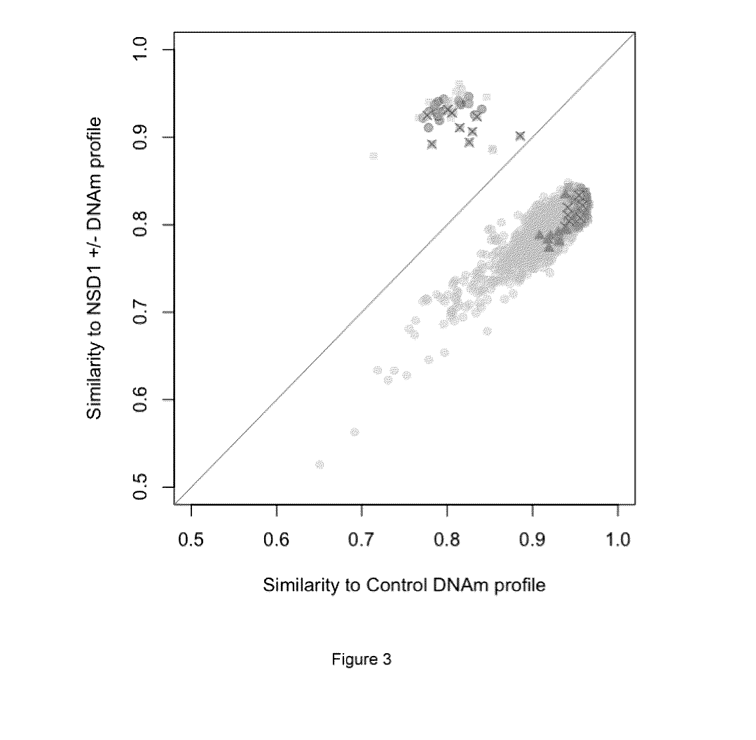

The sensitivity was 100%, as each of the SS subjects in the replication cohort received a positive SS score (FIG. 3).

To further assess the value of the highly specific NSD1^{+/-} DNA methylation signature, the SS DNA methylation profile with the DNA methylation profile of eight subjects with a clinical diagnosis of Weaver syndrome (OMIM 277590) who had confirmed mutations in a different histone methyltransferase (Table 4), Enhancer of Zeste, Drosophila, Homolog 2 or EZH2[Gibson et al, 2012; Tatton-Brown et al, 2011].

All Weaver syndrome patients with EZH2^{+/-} mutations received a strongly negative SS score (between −0.151 and −0.105) and hence were classified confidently as “not Sotos”. Furthermore, hierarchical clustering grouped the Weaver syndrome cases together with the controls, while the SS individuals maintained their distinct cluster, Clearly, the results indicate that the 7K CGs allow the molecular distinction of two clinically overlapping overgrowth syndromes and further demonstrates the specificity of the NSD1^{+/-} DNAm signature for SS (FIG. 3).

### Example 2

### DNAm Signature is Capable of Distinguishing between Pathogenic Variants in NSD1 and Benign Variants in NSD1

The efficiency of the NSD1^{+/-} DNAm signature to functionally classify NSD1 variants of unknown significance (VUS) as pathogenic or benign was investigated. DNA samples from 16 individuals with missense mutations in NSD1 were blinded in terms of inheritance and associated clinical features. As shown in FIG. 3, using average Beta DNA methylation levels at the 7K CGs, a correlation matrix was generated with unsupervised clustering in which nine of 16 samples clustered as a separate group away from the other 7 samples. Both the SS score-based classification (FIG. 3) and the unsupervised hierarchical clustering confirmed that the group of 9 subjects (Group I) carry missense mutations that can clearly be classified as pathogenic since all of them received positive SS score and also clustered with SS subjects. The remaining 7 subjects received negative SS score and clustered with controls indicating benign variant. The classification of variants into benign and pathogenic was compared to 2 other approaches. First, two highly experienced clinical geneticists reviewed the available photos and clinical information for these cases without knowledge of the molecular data for individual cases. Then the predicted pathogenicity of the missense mutations in the cohort utilizing the NSD1^{+/-} DNAm signature was compared to several different prediction algorithms (Table 2) revealing that the DNAm at the NSD1^{+/-} was the most congruent with the clinical diagnosis established by highly experienced clinicians.

Example 3: NSD1^{+/-} methylation signature is evident across multiple cell lineages and the genes associated with the signature are enriched in genes involving neural and cellular development pathways.

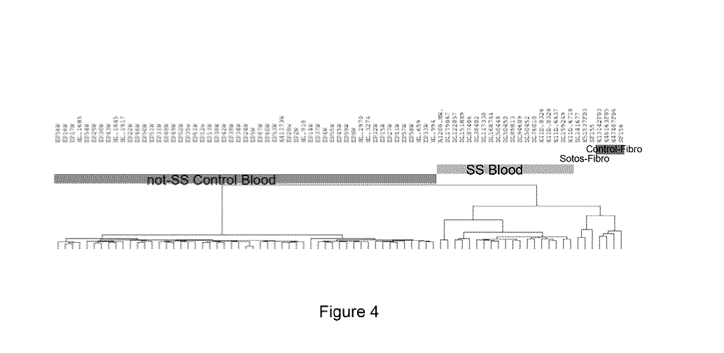

It was hypothesized that NSDI mutations impact the epigenome of multiple cell lineages by generating specific DNA methylation tags at specific genomic regions during embryonic development. Therefore, fibroblast-derived DNA samples from 3 SS subjects with truncating mutations in NSD1 were tested and compared to 4 control fibroblast samples. Using unsupervised hierarchical clustering of the NSD1^{+/-} 7K CGs, it was found that the 3 SS fibroblast clustered separately from control fibroblasts (FIG. 4). Comparing these data to the SS DNAm profile in blood, it was found that a large subset of the CpG sites from the SS specific DNAm signature shared similar DNA methylation profiles in both blood and fibroblast tissues.

Finally, the DNAm signature was investigated for its potential to elucidate the molecular pathophysiology of SS. Analysis of the genomic locations of the 7K CpGs in the NSD1^{+/-} DNA methylation signature showed that the majority of these CpGs mapped to enhancers and CpG island shores. Enrichment analyses for biological and molecular functions using DAVID [Huang da et al, 2009] identified enrichment in neural and cellular development pathways (Benjamini-Hochberg corrected p<0.05) highlighting the main clinical features associated with SS (i.e. overgrowth and developmental delay).

### Example 4

### Sensitivity and Specificity of the NSD1+/− Signature at Various Delta-Beta Cutoffs.

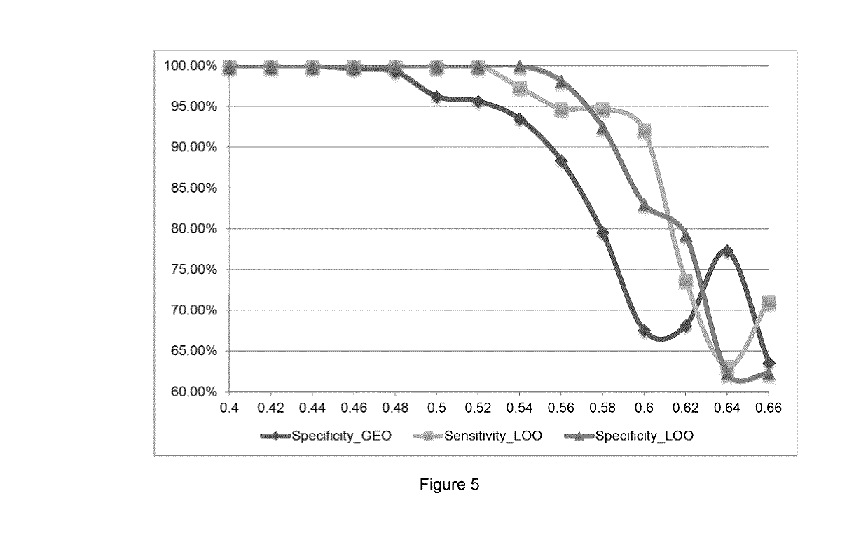

A series of leave-one-out (LOO) cross-validations were performed on the combined dataset (composed of 53 not SS and 36 SS). At each LOO iteration, one sample was removed from the dataset for the subsequent validation step. The remaining samples were used to generate median DNAm profiles for the SS group and for the non-SS group, respectively. Thereafter the previously retained validation sample was classified in terms of its Pearson correlation to either the SS profile or the non-SS profile, using only the significant CpGs as determined by the p-value and delta-beta thresholds. This classification was compared to the sample's true status (SS or non-SS). Based on these LOO results over all 36 (SS)+53 (non-SS) samples, the specificity and sensitivity were estimated for various threshold combinations (Table 5 and FIG. 5). Table 5 includes Recall, Precision and F-measure values. Recall is similar to sensitivity. Precision is calculated as true positives over the sum of true and false positives. F-measure combines precision and recall and is an alternative way to measure sensitivity and specificity.

### Example 5

### DNA Methylation Analysis of Weaver Syndrome Samples

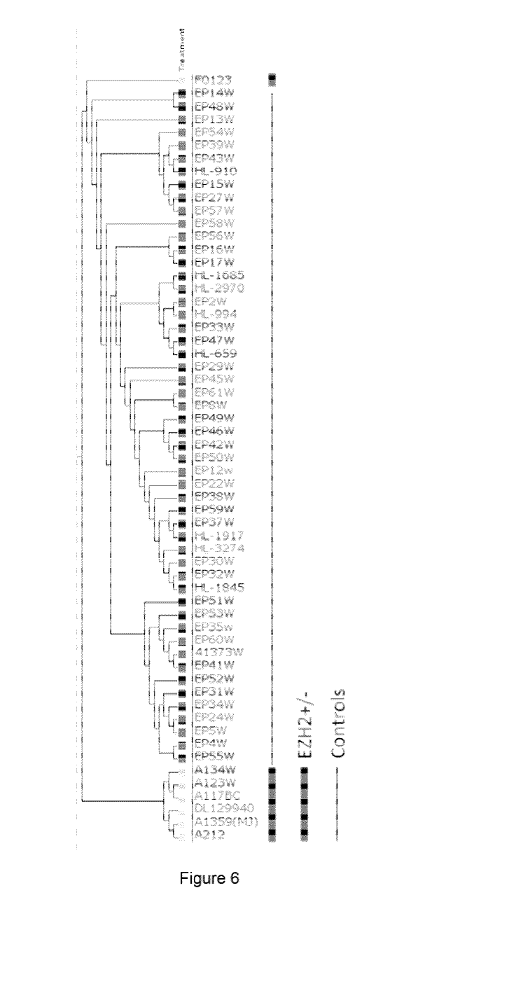

52 control samples and 7 Weaver samples with EZH2+/− variants (Table 6) were used for differential DNA methylation analysis. Three EZH2 variants are described in Table 7. After filtering for non-specific probes as per Chen et al, 2012, 424586 out of 485500 CpG sites were used for the differential DNA methylation analysis. Statistical analysis was performed using Qlucore Omics Explorer. Two group comparisons were performed using two-tailed tests and accounting for gender to assess for differential methylation. At a q<0.05 (Benjamini correction), 3126 CpG sites were identified to be highly significant. The q-value represents the corrected p-value, using the Benjamini Hochberg method to account for multiple testing. FIG. 6 shows unsupervised hierarchical clustering of the EZH2+/− DNA methylation signature.

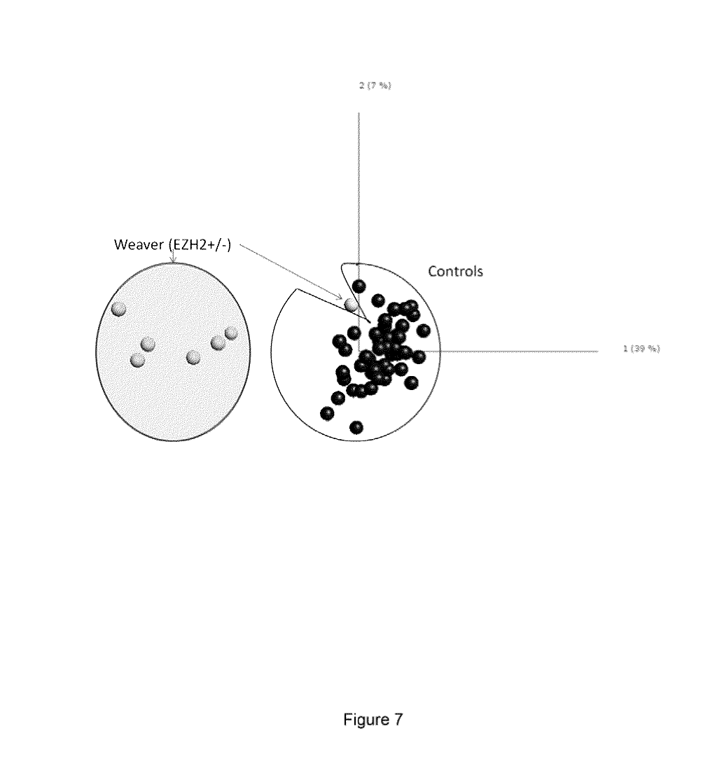

A subset of 6 subjects with EZH2+/− clustered together separate of all control samples and one subject with EZH2+/− (Subject F0123) clustered with the control samples. FIG. 7 shows a principal component analysis revealing a clear separation between EZH2+/− and the controls. Principal Component Analysis (PCA) is a well-established mathematical technique for reducing the dimensionality of data, while keeping as much variation as possible. PCA achieves dimension reduction by creating new, artificial variables called principal components. Each principal component is a linear combination of the observed variables. PCA is an unsupervised method, meaning that no information about groups is used in the dimension reduction. Accordingly, PCA shows a visual representation of the dominant patterns in a data set. By calculating, for example, the first three principal components, and visualizing the samples in this three-dimensional space, a visualization is created containing more of the variance in the original data than any other trio of linear combinations, to provide a three-dimensional sample representation.

Subsequent Insilco analysis using the ClinVar database identified that the variant in F0123 is likely a benign variant. Thus, this data highlights the validity of the EZH2+/− DNA methylation signature for determining the functionality of single nucleotide EZH2 variants.

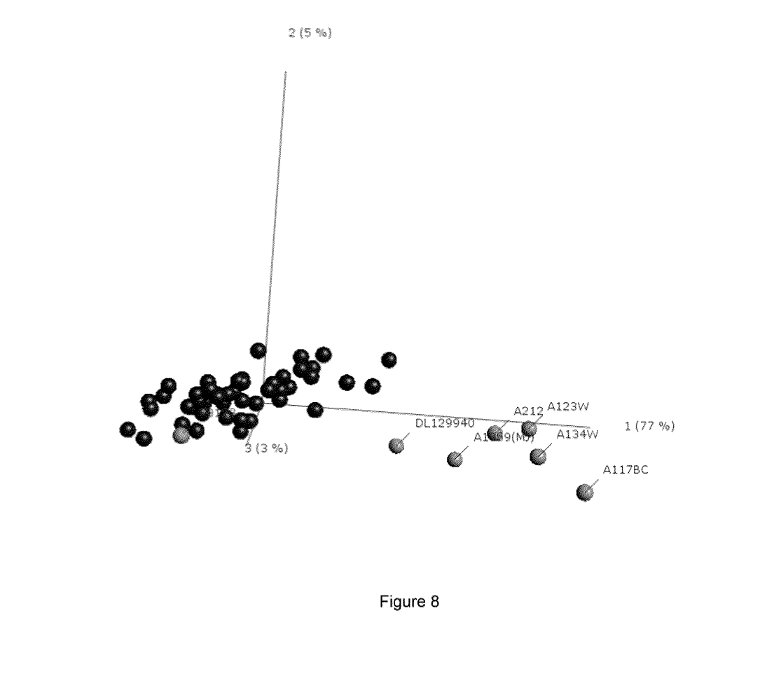

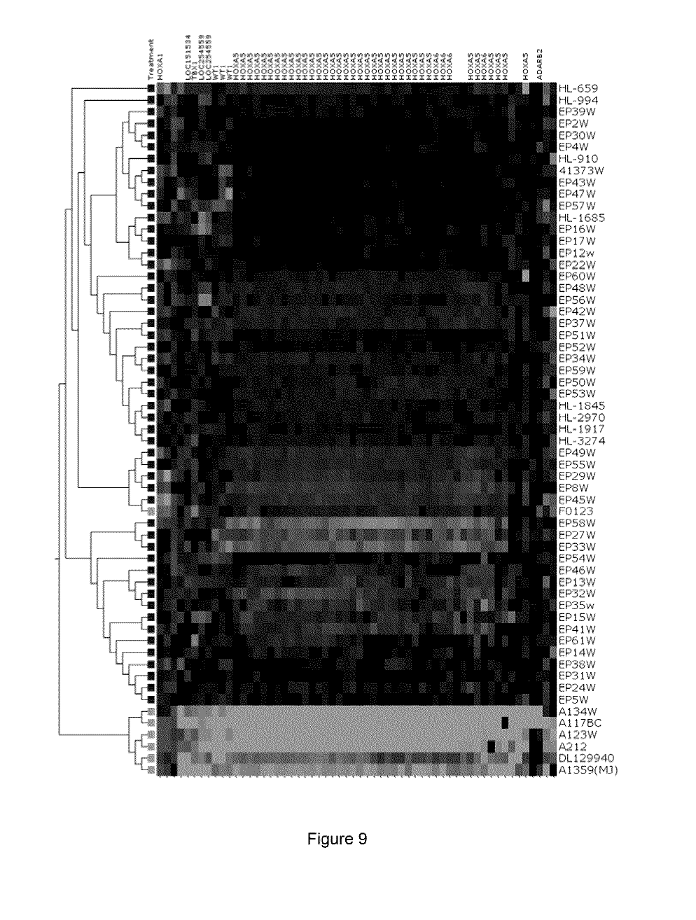

To identify a minimal signature that can classify Weaver subjects separate from control samples, a combination of different fold change threshold and hierarchical clustering was used to select the most relevant CpG sites that will maintain the separation. At a fold change of 1.12 in methylation levels in addition to q<0.05 (Benjamini correction), there are a total of 55 CpG sites contained within 8 genes (FIGS. 8 and 9 and Table 8). A fold change of at least 1.12 at q<0.05 (Benjamini correction) maintains the separation between Weaver samples and control samples when analyzed by hierarchical clustering. In Table 8, both the “difference” and “fold change” are provided for each CpG locus. The “difference” is the “average difference” and represents the difference between the means of methylation level between the Weaver samples and the control samples. The “fold change” is the average difference to the power of 2.

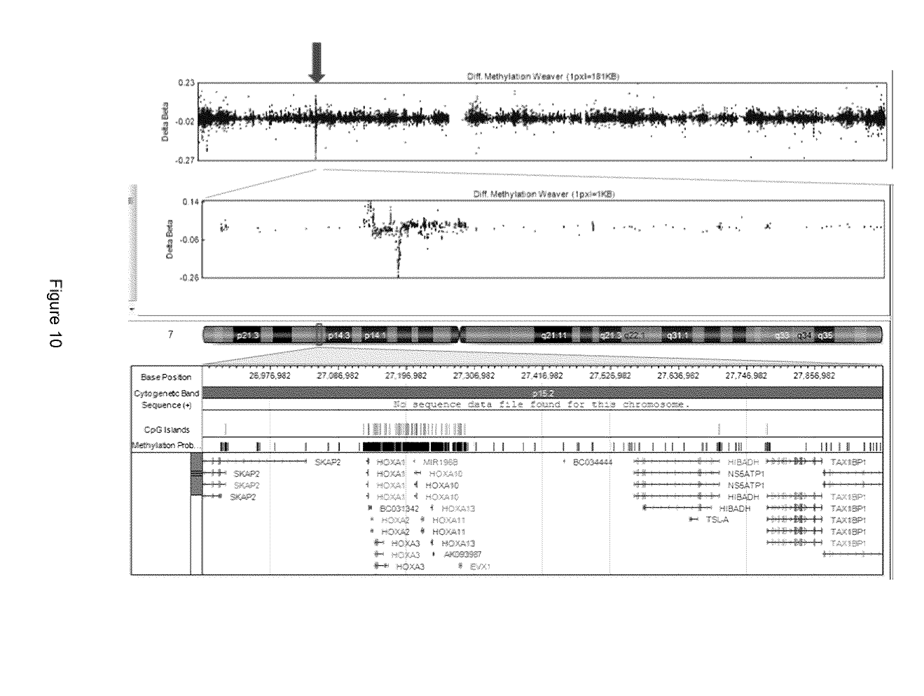

40 of the CpG sites in Table 8 correspond to one gene cluster on chromosome 7 (the HOXA gene cluster) as shown in FIG. 10.

### Example 6

### Sensitivity and Specificity of the EZH2 Signature at Various Delta-Beta Cutoffs.

A series of leave-one-out (LOO) cross-validations are performed on the Weaver dataset. At each LOO iteration, one sample is removed from the dataset for the subsequent validation step. The remaining samples are used to generate median DNAm profiles for the WVS group and for the non-WVS group, respectively. Thereafter the previously retained validation sample is classified in terms of its Pearson correlation to either the WVS profile or the non-WVS profile, using only the significant CpGs as determined by the p-value and delta-beta thresholds. This classification is compared to the sample's true status (WVS or non-WVS). Based on these LOO results, the specificity and sensitivity is estimated for various threshold combinations.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
7085 distinct CpG loci which show a statistically significant (corrected p-value <0.05) difference in methylation levels between
individuals with SS and non-SS controls.

| | | Bonferroni | | | DNA | | | | | |
|---|---|---|---|---|---|---|---|---|---|---|
| | | corrected p- | | | methylation | | | | | |
| Illumina ID | p-value | value | deltaBeta | Absolute deltaBeta | effect | Mean not-SS | Mean SS | Gene Symbol | Genome_Build | Chromosome |
| cg21105875 | 7.13E−10 | 3.03E−04 | −0.667525609 | 0.667525609 | loss | 0.807264945 | 0.139739336 | KDM5B; PCAT6; PCAT6 | 37 | 1 |
| cg23218354 | 7.84E−10 | 3.33E−04 | −0.666484699 | 0.666484699 | loss | 0.792990012 | 0.126505313 | | 37 | 1 |
| cg07600533 | 7.13E−10 | 3.03E−04 | −0.660777038 | 0.660777038 | loss | 0.778205581 | 0.117428544 | KLHDC7B | 37 | 22 |
| cg03133378 | 7.13E−10 | 3.03E−04 | −0.650051516 | 0.650051516 | loss | 0.877130877 | 0.227079362 | LOC100289187; LOC100289187; | 37 | 7 |
| | | | | | | | | LOC100289187 | | |
| cg09056876 | 7.13E−10 | 3.03E−04 | −0.645285393 | 0.645285393 | loss | 0.881861423 | 0.236576029 | | 37 | 6 |
| cg25807487 | 7.13E−10 | 3.03E−04 | −0.641534332 | 0.641534332 | loss | 0.916254198 | 0.274719866 | MRGPRF; MRGPRF | 37 | 11 |
| cg20079298 | 7.13E−10 | 3.03E−04 | −0.636533098 | 0.636533098 | loss | 0.912469498 | 0.2759364 | | 37 | 15 |
| cg09684846 | 7.13E−10 | 3.03E−04 | −0.636128663 | 0.636128663 | loss | 0.871829677 | 0.235701014 | TVP23A; TVP23A | 37 | 16 |
| cg12363903 | 7.13E−10 | 3.03E−04 | −0.633984078 | 0.633984078 | loss | 0.913349802 | 0.279365724 | C5orf66 | 37 | 5 |
| cg02956248 | 7.13E−10 | 3.03E−04 | −0.632348676 | 0.632348676 | loss | 0.921315517 | 0.288966841 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg01996116 | 7.13E−10 | 3.03E−04 | −0.625147247 | 0.625147247 | loss | 0.74575974 | 0.120612492 | HIGD1A; HIGD1A; HIGD1A | 37 | 3 |
| cg24365896 | 7.13E−10 | 3.03E−04 | −0.624559339 | 0.624559339 | loss | 0.923561809 | 0.299002471 | ANO1; ANO1 | 37 | 11 |
| cg13568106 | 7.13E−10 | 3.03E−04 | −0.623671654 | 0.623671654 | loss | 0.882872866 | 0.259201212 | | 37 | 17 |
| cg26863172 | 7.13E−10 | 3.03E−04 | −0.619545045 | 0.619545045 | loss | 0.676123253 | 0.056578207 | TMEM88B | 37 | 1 |
| cg04015962 | 7.13E−10 | 3.03E−04 | −0.618901966 | 0.618901966 | loss | 0.711299494 | 0.092397528 | | 37 | 1 |
| cg00701051 | 7.13E−10 | 3.03E−04 | −0.616076349 | 0.616076349 | loss | 0.877008208 | 0.260931859 | NBPF10; LOC100288142 | 37 | 1 |
| cg03930209 | 7.13E−10 | 3.03E−04 | −0.610314214 | 0.610314214 | loss | 0.644710817 | 0.034396603 | | 37 | 7 |
| cg06704455 | 7.13E−10 | 3.03E−04 | −0.609905089 | 0.609905089 | loss | 0.7984536 | 0.188548511 | ELFN2 | 37 | 22 |
| cg25334934 | 7.13E−10 | 3.03E−04 | −0.60969902 | 0.60969902 | loss | 0.745254668 | 0.135555648 | | 37 | 2 |
| cg23689428 | 7.13E−10 | 3.03E−04 | −0.608262562 | 0.608262562 | loss | 0.846926574 | 0.238664012 | LINC01101; LINC01101 | 37 | 2 |
| cg23359665 | 7.13E−10 | 3.03E−04 | −0.601121501 | 0.601121501 | loss | 0.910036713 | 0.308915212 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg08199758 | 7.13E−10 | 3.03E−04 | −0.596624024 | 0.596624024 | loss | 0.83720693 | 0.240582906 | CLDN9 | 37 | 16 |
| cg23731272 | 1.01E−09 | 4.27E−04 | −0.592967199 | 0.592967199 | loss | 0.772006287 | 0.179039088 | SMAD3 | 37 | 15 |
| cg20021506 | 7.13E−10 | 3.03E−04 | −0.590662775 | 0.590662775 | loss | 0.814213749 | 0.223550974 | | 37 | 6 |
| cg16293892 | 1.19E−09 | 5.07E−04 | −0.589842542 | 0.589842542 | loss | 0.668415877 | 0.078573335 | | 37 | 5 |
| cg15371801 | 7.13E−10 | 3.03E−04 | −0.58893567 | 0.58893567 | loss | 0.617537411 | 0.028601741 | | 37 | 2 |
| cg25844921 | 7.13E−10 | 3.03E−04 | −0.538772866 | 0.588772866 | loss | 0.811419225 | 0.222646359 | MRPS25 | 37 | 3 |
| cg26865747 | 7.13E−10 | 3.03E−04 | −0.587496531 | 0.587496531 | loss | 0.750370338 | 0.162873807 | | 37 | 6 |
| cg07475394 | 7.13E−10 | 3.03E−04 | −0.586398201 | 0.586398201 | loss | 0.799156785 | 0.212758584 | | 37 | 17 |
| cg16005559 | 9.23E−10 | 3.92E−04 | −0.585967254 | 0.585967254 | loss | 0.816837872 | 0.230870618 | | 37 | 11 |
| cg11429111 | 7.13E−10 | 3.03E−04 | −0.584744377 | 0.584744377 | loss | 0.817643836 | 0.232899459 | | 37 | 5 |
| cg00931644 | 7.13E−10 | 3.03E−04 | −0.584691362 | 0.584691362 | loss | 0.819789425 | 0.235098063 | KCTD12 | 37 | 13 |
| cg12846837 | 7.13E−10 | 3.03E−04 | −0.582501041 | 0.582501041 | loss | 0.944654458 | 0.362153418 | SPIDR; SPIDR; SPIDR; SPIDR | 37 | 8 |
| cg10316525 | 7.13E−10 | 3.03E−04 | −0.582297935 | 0.582297935 | loss | 0.953977358 | 0.371679424 | | 37 | 15 |
| cg05820861 | 7.77E−10 | 3.30E−04 | −0.580377701 | 0.580377701 | loss | 0.833947557 | 0.253569855 | C1QTNF9 | 37 | 13 |
| cg08234689 | 8.47E−10 | 3.60E−04 | −0.579808691 | 0.579808691 | loss | 0.712954891 | 0.1331462 | | 37 | 7 |
| cg16717549 | 7.13E−10 | 3.03E−04 | −0.578140386 | 0.578140386 | loss | 0.732366096 | 0.154225711 | AIRE | 37 | 21 |
| cg00982521 | 7.13E−10 | 3.03E−04 | −0.578071702 | 0.578071702 | loss | 0.695241947 | 0.117170245 | | 37 | 6 |
| cg06833564 | 7.13E−10 | 3.03E−04 | −0.575973992 | 0.575973992 | loss | 0.915340657 | 0.339366665 | UBALD1 | 37 | 16 |
| cg07816556 | 7.13E−10 | 3.03E−04 | −0.575612415 | 0.575612415 | loss | 0.776730492 | 0.201118078 | HIST1H1A | 37 | 6 |
| cg25449950 | 9.23E−10 | 3.92E−04 | −0.574895516 | 0.574895516 | loss | 0.92620354 | 0.351308024 | SOD3 | 37 | 4 |
| cg18689454 | 7.13E−10 | 3.03E−04 | −0.574305199 | 0.574305199 | loss | 0.723036508 | 0.148731309 | AIRE | 37 | 21 |
| cg09660878 | 9.23E−10 | 3.92E−04 | −0.571790995 | 0.571790995 | loss | 0.754144621 | 0.182353626 | | 37 | 5 |
| cg12109823 | 7.13E−10 | 3.03E−04 | −0.571660985 | 0.571660985 | loss | 0.732346745 | 0.16068576 | | 37 | 8 |
| cg11291003 | 7.13E−10 | 3.03E−04 | −0.570844179 | 0.570844179 | loss | 0.941511974 | 0.370667794 | C5orf66 | 37 | 5 |
| cg18315060 | 7.13E−10 | 3.03E−04 | −0.570043693 | 0.570043693 | loss | 0.833445323 | 0.263401629 | | 37 | 6 |
| cg26942432 | 7.13E−10 | 3.03E−04 | −0.569691687 | 0.569691687 | loss | 0.721414891 | 0.151723204 | | 37 | 15 |
| cg00186462 | 7.13E−10 | 3.03E−04 | −0.566811985 | 0.566811985 | loss | 0.829719064 | 0.262907079 | GPR128 | 37 | 3 |
| cg20422417 | 7.13E−10 | 3.03E−04 | −0.566475085 | 0.566475085 | loss | 0.663577206 | 0.097102121 | | 37 | 2 |
| cg06729532 | 7.13E−10 | 3.03E−04 | −0.566232486 | 0.566232486 | loss | 0.761560217 | 0.195327731 | | 37 | 19 |
| cg22344745 | 7.13E−10 | 3.03E−04 | −0.564662757 | 0.564662757 | loss | 0.604880411 | 0.040217654 | | 37 | 1 |
| cg03274456 | 7.13E−10 | 3.03E−04 | −0.564049015 | 0.564049015 | loss | 0.848262315 | 0.2842133 | AHNAK | 37 | 11 |
| cg21795221 | 7.13E−10 | 3.03E−04 | −0.561920733 | 0.561920733 | loss | 0.761334462 | 0.199413729 | | 37 | 6 |
| cg02486855 | 8.47E−10 | 3.60E−04 | −0.561237275 | 0.561237275 | loss | 0.793629379 | 0.232392104 | SMAD3 | 37 | 15 |
| cg26465389 | 7.13E−10 | 3.03E−04 | −0.559114632 | 0.559114632 | loss | 0.874881691 | 0.315767059 | | 37 | 10 |
| cg13504434 | 7.13E−10 | 3.03E−04 | −0.556328676 | 0.556328676 | loss | 0.771611215 | 0.215282539 | CABP1; CABP1; CABP1 | 37 | 12 |
| cg08482837 | 7.13E−10 | 3.03E−04 | −0.555827209 | 0.555827209 | loss | 0.77634637 | 0.220519161 | | 37 | 2 |
| cg01761236 | 7.13E−10 | 3.03E−04 | −0.555059387 | 0.555059387 | loss | 0.647741617 | 0.09268223 | | 37 | 14 |
| cg25968569 | 7.13E−10 | 3.03E−04 | −0.553658557 | 0.553658557 | loss | 0.747897832 | 0.194239275 | KCTD12 | 37 | 13 |
| cg14220678 | 7.12E−10 | 3.02E−04 | −0.553450612 | 0.553450612 | loss | 0.876737306 | 0.323286694 | CCDC17; CCDC17 | 37 | 1 |
| cg19092981 | 1.02E−09 | 4.33E−04 | −0.553279608 | 0.553279608 | loss | 0.649049673 | 0.095770065 | TBX1; TBX1; TBX1 | 37 | 22 |
| cg16615454 | 7.13E−10 | 3.03E−04 | −0.552314944 | 0.552314944 | loss | 0.854998332 | 0.302683388 | ASIC1; ASIC1; ASIC1; ASIC1 | 37 | 12 |
| cg08620329 | 7.13E−10 | 3.03E−04 | −0.551737635 | 0.551737635 | loss | 0.781145647 | 0.229408012 | | 37 | 8 |
| cg02781088 | 7.13E−10 | 3.03E−04 | −0.551124829 | 0.551124829 | loss | 0.906738864 | 0.355614035 | RGPD8; RGPD5 | 37 | 2 |
| cg12290671 | 7.13E−10 | 3.03E−04 | −0.550850153 | 0.550850153 | loss | 0.811936258 | 0.261086106 | LOC100289187; LOC100289187; | 37 | 7 |
| | | | | | | | | LOC100289187 | | |
| cg07292773 | 7.13E−10 | 3.03E−04 | −0.549533405 | 0.549533405 | loss | 0.74225417 | 0.192720765 | | 37 | 6 |
| cg05225634 | 7.13E−10 | 3.03E−04 | −0.548557478 | 0.548557478 | loss | 0.864180555 | 0.315623076 | | 37 | 6 |
| cg25646708 | 7.13E−10 | 3.03E−04 | −0.546157046 | 0.546157046 | loss | 0.662232438 | 0.116075392 | | 37 | 3 |
| cg13502125 | 7.13E−10 | 3.03E−04 | −0.545925023 | 0.545925023 | loss | 0.660020989 | 0.114095965 | NBPF8; NBPF8; NBPF8 | 37 | 1 |
| cg10690919 | 7.13E−10 | 3.03E−04 | −0.545905218 | 0.545905218 | loss | 0.930613247 | 0.384708029 | WFIKKN2 | 37 | 17 |
| cg15477144 | 7.13E−10 | 3.03E−04 | −0.545630925 | 0.545630925 | loss | 0.662839649 | 0.117208724 | FAM118A; FAM118A | 37 | 22 |
| cg12403142 | 7.13E−10 | 3.03E−04 | −0.545151071 | 0.545151071 | loss | 0.896332994 | 0.351181924 | | 37 | 1 |
| cg02649698 | 7.13E−10 | 3.03E−04 | −0.544792749 | 0.544792749 | loss | 0.930792155 | 0.385999406 | | 37 | 10 |
| cg11647108 | 7.13E−10 | 3.03E−04 | −0.54464198 | 0.54464198 | loss | 0.809088621 | 0.264446641 | LINC00857 | 37 | 10 |
| cg26915952 | 7.13E−10 | 3.03E−04 | −0.54226202 | 0.54226202 | loss | 0.66315297 | 0.120890949 | | 37 | 1 |
| cg06686742 | 7.13E−10 | 3.03E−04 | −0.541250424 | 0.541250424 | loss | 0.621930428 | 0.080680004 | CERS4 | 37 | 19 |
| cg03962527 | 7.13E−10 | 3.03E−04 | −0.540895316 | 0.540895316 | loss | 0.673987736 | 0.133092419 | ADD3; ADD3; ADD3; ADD3- | 37 | 10 |
| | | | | | | | | AS1 | | |
| cg22249529 | 7.13E−10 | 3.03E−04 | −0.5403968 | 0.5403968 | loss | 0.68848847 | 0.148091669 | KLHDC7B | 37 | 22 |
| cg26153054 | 7.13E−10 | 3.03E−04 | −0.539355044 | 0.539355044 | loss | 0.777373781 | 0.238018737 | | 37 | 2 |
| cg00594854 | 7.13E−10 | 3.03E−04 | −0.53857275 | 0.53857275 | loss | 0.694847202 | 0.156274452 | | 37 | 17 |
| cg19817652 | 1.73E−09 | 7.34E−04 | −0.537740329 | 0.537740329 | loss | 0.737056481 | 0.199316152 | C17orf98 | 37 | 17 |
| cg18373855 | 7.13E−10 | 3.03E−04 | −0.537557253 | 0.537557253 | loss | 0.653673651 | 0.116116398 | | 37 | 6 |
| cg04044120 | 1.30E−09 | 5.52E−04 | −0.537078965 | 0.537078965 | loss | 0.706126825 | 0.169047859 | DNAJB13 | 37 | 11 |
| cg26643967 | 7.13E−10 | 3.03E−04 | −0.535748578 | 0.535748678 | loss | 0.604694723 | 0.068946045 | SDHAF1 | 37 | 19 |
| cg25317631 | 7.13E−10 | 3.03E−04 | −0.530619834 | 0.530619834 | loss | 0.687656336 | 0.157036502 | | 37 | 11 |
| cg10999992 | 7.13E−10 | 3.03E−04 | −0.529714325 | 0.529714325 | loss | 0.892853213 | 0.363138888 | H1F0 | 37 | 22 |
| cg26576890 | 7.13E−10 | 3.03E−04 | −0.529356704 | 0.529356704 | loss | 0.916157192 | 0.386800488 | | 37 | 6 |
| cg26070540 | 7.13E−10 | 3.03E−04 | −0.528475678 | 0.528475678 | loss | 0.771644183 | 0.243168505 | COX4I2 | 37 | 20 |
| cg12131208 | 7.13E−10 | 3.03E−04 | −0.52800963 | 0.52800963 | loss | 0.781655789 | 0.253646159 | C17orf64 | 37 | 17 |
| cg18424208 | 7.13E−10 | 3.03E−04 | −0.527447954 | 0.527447954 | loss | 0.83411256 | 0.306664606 | CDX1 | 37 | 5 |
| cg21778193 | 7.13E−10 | 3.03E−04 | −0.526819811 | 0.526819811 | loss | 0.835847211 | 0.3090274 | | 37 | 1 |
| cg23381884 | 7.13E−10 | 3.03E−04 | −0.525560281 | 0.525560281 | loss | 0.797794545 | 0.272234265 | | 37 | 6 |
| cg08043592 | 7.13E−10 | 3.03E−04 | −0.525310235 | 0.525310235 | loss | 0.861637958 | 0.336327724 | GALP; GALP | 37 | 19 |
| cg02153814 | 7.13E−10 | 3.03E−04 | −0.524897652 | 0.524897652 | loss | 0.761393081 | 0.236495429 | | 37 | 3 |
| cg27535677 | 7.13E−10 | 3.03E−04 | −0.524734494 | 0.524734494 | loss | 0.71632333 | 0.191588836 | | 37 | 6 |
| cg04358214 | 7.13E−10 | 3.03E−04 | −0.524279247 | 0.524279247 | loss | 0.661170626 | 0.136891379 | C16orf70 | 37 | 16 |
| cg07187855 | 7.13E−10 | 3.03E−04 | −0.523020902 | 0.523020902 | loss | 0.714571402 | 0.1915505 | DDR1; DDR1; DDR1 | 37 | 6 |
| cg10502244 | 7.13E−10 | 3.03E−04 | −0.522520791 | 0.522520791 | loss | 0.708996847 | 0.186476056 | COL2A1; COL2A1 | 37 | 12 |
| cg21915678 | 7.13E−10 | 3.03E−04 | −0.52192296 | 0.52192296 | loss | 0.96356156 | 0.4416386 | | 37 | 17 |
| cg26004771 | 7.13E−10 | 3.03E−04 | −0.521469875 | 0.521469875 | loss | 0.55636257 | 0.034892695 | | 37 | 1 |
| cg09614479 | 7.13E−10 | 3.03E−04 | −0.521134622 | 0.521134622 | loss | 0.695134513 | 0.173999891 | LINC00857 | 37 | 10 |
| cg16073143 | 7.13E−10 | 3.03E−04 | −0.52040328 | 0.52040328 | loss | 0.670363309 | 0.149960029 | | 37 | 6 |
| cg04157161 | 7.13E−10 | 3.03E−04 | −0.518564786 | 0.518564786 | loss | 0.907584645 | 0.389019859 | GUCY2D | 37 | 17 |
| cg06154311 | 7.13E−10 | 3.03E−04 | −0.517339367 | 0.517339367 | loss | 0.744439285 | 0.227099918 | RBBP8NL; RBBP8NL | 37 | 20 |
| cg12233487 | 7.13E−10 | 3.03E−04 | −0.515729517 | 0.515729517 | loss | 0.748212975 | 0.232433459 | TMC2 | 37 | 20 |
| cg13471599 | 7.13E−10 | 3.03E−04 | −0.515679857 | 0.515679857 | loss | 0.862393392 | 0.346713535 | | 37 | 12 |
| cg02404974 | 7.13E−10 | 3.03E−04 | −0.515573872 | 0.515573872 | loss | 0.778736119 | 0.263162247 | | 37 | 6 |
| cg08083251 | 7.84E−10 | 3.33E−04 | −0.51464369 | 0.51464369 | loss | 0.63711281 | 0.12246912 | | 37 | 13 |
| cg04561804 | 7.84E−10 | 3.33E−04 | −0.513335931 | 0.513335931 | loss | 0.795612231 | 0.2822763 | SEC62; LOC100128164; LOC100128164 | 37 | 3 |
| cg14167415 | 1.19E−09 | 5.07E−04 | −0.513318481 | 0.513318481 | loss | 0.715788985 | 0.202470504 | | 37 | 5 |
| cg04359418 | 7.13E−10 | 3.03E−04 | −0.511630895 | 0.511630895 | loss | 0.842201177 | 0.330570282 | | 37 | 10 |
| cg26600753 | 7.13E−10 | 3.03E−04 | −0.511626816 | 0.511626816 | loss | 0.889819634 | 0.378192818 | TRIM7; TRIM7; TRIM7; TRIM7; | 37 | 5 |
| | | | | | | | | TRIM7 | | |
| cg05412788 | 7.13E−10 | 3.03E−04 | −0.510851587 | 0.510851587 | loss | 0.89909754 | 0.388245953 | | 37 | 12 |
| cg09593860 | 7.13E−10 | 3.03E−04 | −0.51038869 | 0.51038869 | loss | 0.930696455 | 0.420307765 | ZBTB16 | 37 | 11 |
| cg13614409 | 7.13E−10 | 3.03E−04 | −0.510337893 | 0.510337893 | loss | 0.632001204 | 0.121663311 | TEX26; TEX26-AS1; TEX26- | 37 | 13 |
| | | | | | | | | AS1; TEX26-AS1; TEX26- | | |
| | | | | | | | | AS1 | | |
| cg21811021 | 8.47E−10 | 3.60E−04 | −0.510327301 | 0.510327301 | loss | 0.822038219 | 0.311710918 | | 37 | 4 |
| cg21855361 | 8.47E−10 | 3.60E−04 | −0.51023467 | 0.51023467 | loss | 0.653689543 | 0.143454873 | | 37 | 7 |
| cg17801295 | 7.13E−10 | 3.03E−04 | −0.510175559 | 0.510175559 | loss | 0.872565383 | 0.362389824 | | 37 | 2 |
| cg00990385 | 7.13E−10 | 3.03E−04 | −0.510066251 | 0.510066251 | loss | 0.736727087 | 0.226660835 | CCDC42B | 37 | 12 |
| cg09044186 | 7.13E−10 | 3.03E−04 | −0.509061982 | 0.509061982 | loss | 0.947134847 | 0.438072865 | APOA5; APOA5 | 37 | 11 |
| cg22198853 | 3.87E−09 | 1.64E−03 | −0.50903874 | 0.50903874 | loss | 0.878159176 | 0.369120437 | | 37 | 6 |
| cg25885280 | 7.13E−10 | 3.03E−04 | −0.508239641 | 0.508239641 | loss | 0.677585619 | 0.169345978 | SHANK2 | 37 | 11 |
| cg17604655 | 7.13E−10 | 3.03E−04 | −0.507946482 | 0.507946482 | loss | 0.743487011 | 0.235540529 | | 37 | 7 |
| cg17900689 | 7.13E−10 | 3.03E−04 | −0.506828682 | 0.506828682 | loss | 0.721424406 | 0.214595724 | ZMYND15; ZMYND15; ZMYND15 | 37 | 17 |
| cg21010202 | 7.13E−10 | 3.03E−04 | −0.506476237 | 0.506476237 | loss | 0.754270055 | 0.247793818 | | 37 | 6 |
| cg05168012 | 7.13E−10 | 3.03E−04 | −0.50546475 | 0.50546475 | loss | 0.962566266 | 0.457101516 | | 37 | 2 |
| cg20137746 | 7.13E−10 | 3.03E−04 | −0.505390571 | 0.505390571 | loss | 0.768052694 | 0.262662124 | | 37 | 10 |
| cg17116120 | 7.77E−10 | 3.30E−04 | −0.504680735 | 0.504680735 | loss | 0.671342417 | 0.166661682 | COX4I2 | 37 | 20 |
| cg25438517 | 7.13E−10 | 3.03E−04 | −0.504280656 | 0.504280656 | loss | 0.803769015 | 0.299488359 | | 37 | 5 |
| cg09674170 | 7.13E−10 | 3.03E−04 | −0.503727877 | 0.503727877 | loss | 0.630479521 | 0.126751644 | CLDN9 | 37 | 16 |
| cg00970752 | 7.13E−10 | 3.03E−04 | −0.502724612 | 0.502724612 | loss | 0.665929002 | 0.16320439 | FHAD1 | 37 | 1 |
| cg11117637 | 7.84E−10 | 3.33E−04 | −0.502134769 | 0.502134769 | loss | 0.848691587 | 0.346556818 | CDX1 | 37 | 5 |
| cg06842409 | 7.13E−10 | 3.03E−04 | −0.502047573 | 0.502047573 | loss | 0.676171585 | 0.174124012 | | 37 | 20 |
| cg05516295 | 7.13E−10 | 3.03E−04 | −0.502023255 | 0.502023255 | loss | 0.822340243 | 0.320316988 | C5orf66 | 37 | 5 |
| cg10365562 | 7.13E−10 | 3.03E−04 | −0.501987404 | 0.501987404 | loss | 0.661048511 | 0.159061107 | TDGF1 | 37 | 3 |
| cg14706297 | 7.13E−10 | 3.03E−04 | −0.501768111 | 0.501768111 | loss | 0.655157719 | 0.153389608 | | 37 | 2 |
| cg10676327 | 7.13E−10 | 3.03E−04 | −0.50117058 | 0.50117058 | loss | 0.616161792 | 0.114991212 | | 37 | 4 |
| cg19196401 | 7.84E−10 | 3.33E−04 | −0.501106703 | 0.501106703 | loss | 0.647275154 | 0.146168451 | DDO; DDO | 37 | 6 |
| cg02632490 | 4.31E−09 | 1.83E−03 | −0.500749319 | 0.500749319 | loss | 0.885434106 | 0.384684787 | GRID1; MIR346; MIR346 | 37 | 10 |
| cg26707664 | 7.13E−10 | 3.03E−04 | −0.499598654 | 0.499598654 | loss | 0.693348719 | 0.193750065 | | 37 | 18 |
| cg06423211 | 1.10E−09 | 4.65E−04 | −0.49879343 | 0.49879343 | loss | 0.678280921 | 0.179487491 | | 37 | 19 |
| cg25257677 | 7.13E−10 | 3.03E−04 | −0.498557951 | 0.498557951 | loss | 0.691492645 | 0.192934695 | SLC39A14; SLC39A14; SLC39A14; | 37 | 8 |
| | | | | | | | | SLC39A14 | | |
| cg19094530 | 7.13E−10 | 3.03E−04 | −0.498338378 | 0.498338378 | loss | 0.72858486 | 0.230246482 | | 37 | 10 |
| cg12642717 | 7.13E−10 | 3.03E−04 | −0.497407554 | 0.497407554 | loss | 0.98224043 | 0.484832876 | CIDEA; CIDEA | 37 | 18 |
| cg20737388 | 1.83E−09 | 7.75E−04 | −0.496028632 | 0.496028632 | loss | 0.659817425 | 0.163788792 | DNAJB13 | 37 | 11 |
| cg04220541 | 7.13E−10 | 3.03E−04 | −0.495852391 | 0.495852391 | loss | 0.925245368 | 0.429392976 | SPIDR; SPIDR; SPIDR; SPIDR | 37 | 8 |
| cg26695758 | 7.13E−10 | 3.03E−04 | −0.495744913 | 0.495744913 | loss | 0.799232749 | 0.303487836 | TNXB | 37 | 6 |
| cg06415891 | 7.84E−10 | 3.33E−04 | −0.495658668 | 0.495658668 | loss | 0.755529744 | 0.259871076 | | 37 | 1 |
| cg13939055 | 7.13E−10 | 3.03E−04 | −0.495367476 | 0.495367476 | loss | 0.677413936 | 0.182046459 | LOC339529 | 37 | 1 |
| cg20816361 | 7.13E−10 | 3.03E−04 | −0.494006923 | 0.494006923 | loss | 0.761703723 | 0.2676968 | | 37 | 1 |
| cg00443981 | 7.13E−10 | 3.03E−04 | −0.493415997 | 0.493415997 | loss | 0.911612521 | 0.418196524 | C17orf64 | 37 | 17 |
| cg27500148 | 7.13E−10 | 3.03E−04 | −0.493414857 | 0.493414857 | loss | 0.904364445 | 0.410949588 | RPL23 | 37 | 17 |
| cg17976205 | 7.13E−10 | 3.03E−04 | −0.492894277 | 0.492894277 | loss | 0.820588989 | 0.327694712 | RBBP8NL | 37 | 20 |
| cg09634707 | 7.13E−10 | 3.03E−04 | −0.492206711 | 0.492206711 | loss | 0.627276017 | 0.135069306 | | 37 | 10 |
| cg11372436 | 1.21E−09 | 5.15E−04 | −0.491462365 | 0.491462365 | loss | 0.922202083 | 0.430739718 | SOD3 | 37 | 4 |
| cg07537443 | 7.13E−10 | 3.03E−04 | −0.490610195 | 0.490610195 | loss | 0.641119794 | 0.150509599 | NGFR | 37 | 17 |
| cg00549574 | 7.13E−10 | 3.03E−04 | −0.490600735 | 0.490600735 | loss | 0.859740158 | 0.369139424 | PRPSAP1 | 37 | 17 |
| cg22300566 | 7.13E−10 | 3.03E−04 | −0.489283704 | 0.489283704 | loss | 0.853773945 | 0.364490241 | | 37 | 6 |
| cg05308495 | 7.13E−10 | 3.03E−04 | −0.488620742 | 0.488620742 | loss | 0.636988958 | 0.148368216 | CACNG6; CACNG6; CACNG6; | 37 | 19 |
| | | | | | | | | CACNG6 | | |
| cg06693983 | 7.13E−10 | 3.03E−04 | −0.488569384 | 0.488569384 | loss | 0.764714796 | 0.276145412 | TMEM190 | 37 | 19 |
| cg08379987 | 7.13E−10 | 3.03E−04 | −0.488291735 | 0.488291735 | loss | 0.698124374 | 0.209832638 | TEX26; TEX26-AS1; TEX26- | 37 | 13 |
| | | | | | | | | AS1 | | |
| cg09692695 | 7.13E−10 | 3.03E−04 | −0.487350814 | 0.487350814 | loss | 0.792935355 | 0.305584541 | RTKN; RTKN; RTKN | 37 | 2 |
| cg04202002 | 7.13E−10 | 3.03E−04 | −0.487171358 | 0.487171358 | loss | 0.781307823 | 0.294136465 | | 37 | 2 |
| cg10635895 | 7.13E−10 | 3.03E−04 | −0.486421496 | 0.486421496 | loss | 0.598641794 | 0.112220299 | CACNA1C; CACNA1C; CACNA1C; | 37 | 12 |
| | | | | | | | | CACNA1C; CACNA1C; | | |
| | | | | | | | | CACNA1C; CACNA1C; CACNA1C; | | |
| | | | | | | | | CACNA1C; CACNA1C; | | |
| | | | | | | | | CACNA1C; CACNA1C; CACNA1C; | | |
| | | | | | | | | CACNA1C; CACNA1C; | | |
| | | | | | | | | CACNA1C; CACNA1C; | | |
| | | | | | | | | CACNA1C; CACNA1C; CACNA1C; | | |
| | | | | | | | | CACNA1C; CACNA1C; | | |
| | | | | | | | | CACNA1C; | | |
| cg07405182 | 7.13E−10 | 3.03E−04 | −0.485768588 | 0.485768588 | loss | 0.665093421 | 0.179324833 | | 37 | 1 |
| cg14429919 | 8.47E−10 | 3.60E−04 | −0.485191383 | 0.485191383 | loss | 0.831063725 | 0.345872341 | | 37 | 6 |
| cg00091064 | 7.13E−10 | 3.03E−04 | −0.484681796 | 0.484681796 | loss | 0.797603396 | 0.3129216 | | 37 | 6 |
| cg10555383 | 8.47E−10 | 3.60E−04 | −0.4833286 | 0.4833286 | loss | 0.625226862 | 0.141898262 | LYNX1; LYNX1; LYNX1 | 37 | 8 |
| cg24574963 | 7.13E−10 | 3.03E−04 | −0.482990798 | 0.482990798 | loss | 0.665378857 | 0.182388058 | | 37 | 7 |
| cg18568930 | 7.13E−10 | 3.03E−04 | −0.482176979 | 0.482176979 | loss | 0.713816874 | 0.231639894 | IGLONS | 37 | 19 |
| cg17724412 | 7.13E−10 | 3.03E−04 | −0.481905265 | 0.481905265 | loss | 0.8846546 | 0.402749335 | LOC401242 | 37 | 6 |
| cg14652587 | 7.13E−10 | 3.03E−04 | −0.481862975 | 0.481862975 | loss | 0.586174245 | 0.10431127 | | 37 | 17 |
| cg21406402 | 7.13E−10 | 3.03E−04 | −0.481843836 | 0.481843836 | loss | 0.628769079 | 0.146925243 | LINC01101; LINC01101 | 37 | 2 |
| cg23924370 | 7.13E−10 | 3.03E−04 | −0.481757897 | 0.481757897 | loss | 0.691106979 | 0.209349082 | | 37 | 2 |
| cg00643333 | 7.13E−10 | 3.03E−04 | −0.481036424 | 0.481036424 | loss | 0.661059177 | 0.180022753 | FAM118A; FAM118A | 37 | 22 |
| cg17034331 | 7.13E−10 | 3.03E−04 | −0.480900351 | 0.480900351 | loss | 0.804161104 | 0.323260753 | | 37 | 5 |
| cg01952185 | 7.13E−10 | 3.03E−04 | −0.48077913 | 0.48077913 | loss | 0.702348636 | 0.221569506 | | 37 | 5 |
| cg00823843 | 7.13E−10 | 3.03E−04 | −0.480460021 | 0.480460021 | loss | 0.664236549 | 0.183776528 | LOC339529 | 37 | 1 |
| cg00944421 | 7.13E−10 | 3.03E−04 | −0.47993921 | 0.47993921 | loss | 0.883873545 | 0.403934335 | ESRP2 | 37 | 16 |
| cg24550212 | 7.13E−10 | 3.03E−04 | −0.47977371 | 0.47977371 | loss | 0.953082304 | 0.473308594 | OCSTAMP | 37 | 20 |
| cg19940077 | 7.13E−10 | 3.03E−04 | −0.479647685 | 0.479647685 | loss | 0.883282579 | 0.403634894 | GPR25 | 37 | 1 |
| cg18409649 | 7.13E−10 | 3.03E−04 | −0.479423368 | 0.479423368 | loss | 0.633053708 | 0.15363034 | | 37 | 7 |
| cg03687532 | 7.13E−10 | 3.03E−04 | −0.479318855 | 0.479318855 | loss | 0.872776708 | 0.393457853 | | 37 | 16 |
| cg18121224 | 7.13E−10 | 3.03E−04 | −0.478885805 | 0.478885805 | loss | 0.596801702 | 0.117915897 | NSD1; NSD1 | 37 | 5 |
| cg27102629 | 7.13E−10 | 3.03E−04 | −0.478634384 | 0.478634384 | loss | 0.784942996 | 0.306308612 | | 37 | 10 |
| cg01342115 | 7.13E−10 | 3.03E−04 | −0.478472699 | 0.478472699 | loss | 0.612970064 | 0.134497365 | | 37 | 3 |
| cg11559198 | 7.13E−10 | 3.03E−04 | −0.477502019 | 0.477502019 | loss | 0.558613923 | 0.081111904 | | 37 | 2 |
| cg07581811 | 7.13E−10 | 3.03E−04 | −0.477322732 | 0.477322732 | loss | 0.977099379 | 0.499776647 | | 37 | 3 |
| cg00270878 | 1.01E−09 | 4.27E−04 | −0.476345614 | 0.476345614 | loss | 0.738598138 | 0.262252524 | SLC22A11 | 37 | 11 |
| cg14825735 | 6.89E−09 | 2.93E−03 | −0.476178885 | 0.476178885 | loss | 0.753666892 | 0.277488008 | | 37 | 6 |
| cg21411366 | 8.47E−10 | 3.60E−04 | −0.475961621 | 0.475961621 | loss | 0.838403662 | 0.362442041 | SOD3 | 37 | 4 |
| cg20703261 | 7.13E−10 | 3.03E−04 | −0.475907132 | 0.475907132 | loss | 0.852843491 | 0.376935359 | | 37 | 6 |
| cg19402939 | 7.13E−10 | 3.03E−04 | −0.475839 | 0.475839 | loss | 0.708699842 | 0.232860841 | CHRD | 37 | 3 |
| cg00524708 | 7.13E−10 | 3.03E−04 | −0.475268477 | 0.475268477 | loss | 0.573403409 | 0.098134932 | PRSS36; PRSS36; PRSS36 | 37 | 16 |
| cg02123734 | 8.47E−10 | 3.60E−04 | −0.474942928 | 0.474942928 | loss | 0.601035045 | 0.126092117 | | 37 | 10 |
| cg27362048 | 7.13E−10 | 3.03E−04 | −0.474878476 | 0.474878476 | loss | 0.886756611 | 0.411878135 | APOA5; APOA5 | 37 | 11 |
| cg13713218 | 9.23E−10 | 3.92E−04 | −0.474335977 | 0.474335977 | loss | 0.76658274 | 0.292246762 | LOC100499484; LOC100499484- | 37 | 9 |
| | | | | | | | | C9ORF174; LOC100499484- | | |
| | | | | | | | | C9ORF174; LOC100499484- | | |
| | | | | | | | | C9ORF174 | | |
| cg23634477 | 1.99E−09 | 8.43E−04 | −0.473733333 | 0.473733333 | loss | 0.872531257 | 0.398797924 | | 37 | 6 |
| cg25066665 | 6.35E−09 | 2.70E−03 | −0.473619952 | 0.473619952 | loss | 0.823456134 | 0.349836182 | RPRD2 | 37 | 1 |
| cg14708990 | 7.13E−10 | 3.03E−04 | −0.473464365 | 0.473464365 | loss | 0.7996888 | 0.326224435 | MYO7A; MYO7A; MYO7A | 37 | 11 |
| cg06582782 | 2.48E−08 | 1.05E−02 | −0.473451168 | 0.473451168 | loss | 0.895320857 | 0.421869688 | | 37 | 12 |
| cg09089451 | 7.13E−10 | 3.03E−04 | −0.472869572 | 0.472869572 | loss | 0.867145325 | 0.394275753 | KRBA1 | 37 | 7 |
| cg11450541 | 7.13E−10 | 3.03E−04 | −0.472813207 | 0.472813207 | loss | 0.726727211 | 0.253914004 | ANKRD20A19P | 37 | 13 |
| cg23505823 | 1.42E−09 | 6.01E−04 | −0.472678922 | 0.472678922 | loss | 0.907311798 | 0.434632876 | HCAR1 | 37 | 12 |
| cg19772114 | 7.13E−10 | 3.03E−04 | −0.472518926 | 0.472518926 | loss | 0.940760396 | 0.468241471 | LOC401242 | 37 | 6 |
| cg11012412 | 3.78E−09 | 1.61E−03 | −0.471959241 | 0.471959241 | loss | 0.602655628 | 0.130696387 | | 37 | 5 |
| cg02254407 | 1.73E−09 | 7.34E−04 | −0.471842315 | 0.471842315 | loss | 0.857284962 | 0.385442647 | PLEKHB1; PLEKHB1; PLEKHB1; | 37 | 11 |
| | | | | | | | | PLEKHB1 | | |
| cg17453840 | 7.77E−10 | 3.30E−04 | −0.471330912 | 0.471330912 | loss | 0.842043147 | 0.370712235 | CPEB1; LOC283692 | 37 | 15 |
| cg08772588 | 7.13E−10 | 3.03E−04 | −0.471321639 | 0.471321639 | loss | 0.788704645 | 0.317383006 | SSC5D; SSC5D | 37 | 19 |
| cg04539705 | 7.13E−10 | 3.03E−04 | −0.470931496 | 0.470931496 | loss | 0.702726343 | 0.231794847 | PRSS56 | 37 | 2 |
| cg04942251 | 7.13E−10 | 3.03E−04 | −0.470711918 | 0.470711918 | loss | 0.650031823 | 0.179319905 | | 37 | 11 |
| cg09288885 | 7.13E−10 | 3.03E−04 | −0.47056995 | 0.47056995 | loss | 0.737324179 | 0.266754229 | | 37 | 16 |
| cg10365886 | 1.01E−09 | 4.27E−04 | −0.470259963 | 0.470259963 | loss | 0.693914304 | 0.223654341 | TNXB | 37 | 6 |
| cg24524285 | 7.13E−10 | 3.03E−04 | −0.47019212 | 0.47019212 | loss | 0.668520275 | 0.198328156 | NRXN2; NRXN2; NRXN2 | 37 | 11 |
| cg21543589 | 7.13E−10 | 3.03E−04 | −0.469780067 | 0.469780067 | loss | 0.710939185 | 0.241159118 | COX4I2 | 37 | 20 |
| cg15674899 | 7.13E−10 | 3.03E−04 | −0.469385767 | 0.469385767 | loss | 0.796817038 | 0.327431271 | | 37 | 5 |
| cg07242540 | 7.13E−10 | 3.03E−04 | −0.469367149 | 0.469367149 | loss | 0.83571986 | 0.366352712 | | 37 | 1 |
| cg20084219 | 7.84E−10 | 3.33E−04 | −0.467980491 | 0.467980491 | loss | 0.637834896 | 0.169854405 | C17orf98; C17orf98 | 37 | 17 |
| cg02757970 | 7.13E−10 | 3.03E−04 | −0.467344459 | 0.467344459 | loss | 0.658774036 | 0.191429576 | | 37 | 2 |
| cg12133451 | 7.13E−10 | 3.03E−04 | −0.467171452 | 0.467171452 | loss | 0.658923874 | 0.191752422 | | 37 | 1 |
| cg01856384 | 7.13E−10 | 3.03E−04 | −0.466791569 | 0.466791569 | loss | 0.620338198 | 0.153546629 | | 37 | 6 |
| cg26209990 | 8.47E−10 | 3.60E−04 | −0.466666726 | 0.466666726 | loss | 0.674669058 | 0.208002333 | | 37 | 7 |
| cg11923631 | 7.13E−10 | 3.03E−04 | −0.466428107 | 0.466428107 | loss | 0.56794094 | 0.101512833 | AIRE | 37 | 21 |
| cg06303238 | 7.13E−10 | 3.03E−04 | −0.466198437 | 0.466198437 | loss | 0.818940013 | 0.352741576 | SALL4; SALL4 | 37 | 20 |
| cg03063309 | 1.19E−09 | 5.07E−04 | −0.465951649 | 0.465951649 | loss | 0.70832246 | 0.242370812 | SOD3 | 37 | 4 |
| cg05133205 | 7.13E−10 | 3.03E−04 | −0.465826103 | 0.465826103 | loss | 0.589031404 | 0.123205301 | PPT2; PPT2; PPT2; LOC100507547; | 37 | 6 |
| | | | | | | | | LOC100507547; LOC100507547; | | |
| | | | | | | | | LOC100507547; | | |
| | | | | | | | | PPT2-EGFL8 | | |
| cg05630111 | 1.73E−09 | 7.34E−04 | −0.465576373 | 0.465576373 | loss | 0.631265558 | 0.165689186 | CERS2; CERS2 | 37 | 1 |
| cg20625967 | 7.13E−10 | 3.03E−04 | −0.465325158 | 0.465325158 | loss | 0.952837511 | 0.487512353 | PLA2G4E | 37 | 15 |
| cg10854819 | 6.10E−09 | 2.59E−03 | −0.464742893 | 0.464742893 | loss | 0.726900648 | 0.262157755 | LOC283710 | 37 | 15 |
| cg27251412 | 7.13E−10 | 3.03E−04 | −0.464302584 | 0.464302584 | loss | 0.592549508 | 0.128246924 | AIRE | 37 | 21 |
| cg23280506 | 5.51E−09 | 2.34E−03 | −0.463890781 | 0.463890781 | loss | 0.57466165 | 0.110770869 | | 37 | 17 |
| cg17978631 | 7.13E−10 | 3.03E−04 | −0.463811339 | 0.463811339 | loss | 0.898171462 | 0.434360124 | | 37 | 3 |
| cg17261676 | 7.13E−10 | 3.03E−04 | −0.463287206 | 0.463287206 | loss | 0.925896858 | 0.462609653 | | 37 | 6 |
| cg20701556 | 7.13E−10 | 3.03E−04 | −0.463159568 | 0.463159568 | loss | 0.735505121 | 0.272346553 | RWDD3; RWDD3; TMEM56- | 37 | 1 |
| | | | | | | | | RWDD3; RWDD3; RWDD3; | | |
| | | | | | | | | RWDD3; RWDD3; RWDD3 | | |
| cg21235532 | 7.13E−10 | 3.03E−04 | −0.462858351 | 0.462858351 | loss | 0.653823421 | 0.19096507 | | 37 | 18 |
| cg04159546 | 7.13E−10 | 3.03E−04 | −0.462804201 | 0.462804201 | loss | 0.785128072 | 0.322323871 | TP73; TP73; TP73; TP73; TP73; | 37 | 1 |
| | | | | | | | | TP73 | | |
| cg26010218 | 7.13E−10 | 3.03E−04 | −0.462228449 | 0.462228449 | loss | 0.904977743 | 0.442749294 | | 37 | 4 |
| cg01235820 | 7.13E−10 | 3.03E−04 | −0.462013473 | 0.462013473 | loss | 0.640483087 | 0.178469614 | SCRT2 | 37 | 20 |
| cg20462561 | 9.23E−10 | 3.92E−04 | −0.461667392 | 0.461667392 | loss | 0.727610398 | 0.265943006 | | 37 | 1 |
| cg06689045 | 7.13E−10 | 3.03E−04 | −0.461558205 | 0.461558205 | loss | 0.709427323 | 0.247869118 | | 37 | 3 |
| cg19049754 | 7.13E−10 | 3.03E−04 | −0.461445259 | 0.461445259 | loss | 0.953579836 | 0.492134576 | LINC00857 | 37 | 10 |
| cg06825512 | 7.13E−10 | 3.03E−04 | −0.461327825 | 0.461327825 | loss | 0.748954972 | 0.287627147 | APCDD1 | 37 | 18 |
| cg11985480 | 7.13E−10 | 3.03E−04 | −0.461125107 | 0.461125107 | loss | 0.762637806 | 0.301512699 | LBX2; LBX2-AS1 | 37 | 2 |
| cg25457886 | 7.13E−10 | 3.03E−04 | −0.461071239 | 0.461071239 | loss | 0.572079155 | 0.111007916 | | 37 | 3 |
| cg01205267 | 7.13E−10 | 3.03E−04 | −0.460262812 | 0.460262812 | loss | 0.833072 | 0.372809188 | LRCH1; LRCH1; LRCH1 | 37 | 13 |
| cg03541791 | 7.13E−10 | 3.03E−04 | −0.460172332 | 0.460172332 | loss | 0.885580374 | 0.425408041 | LOC401242 | 37 | 6 |
| cg24024505 | 7.13E−10 | 3.03E−04 | −0.460154791 | 0.460154791 | loss | 0.977614121 | 0.517459329 | LOC401242 | 37 | 6 |
| cg17918558 | 7.13E−10 | 3.03E−04 | −0.459820658 | 0.459820658 | loss | 0.654012758 | 0.1941921 | | 37 | 1 |
| cg00003625 | 7.13E−10 | 3.03E−04 | −0.459601485 | 0.459601485 | loss | 0.869226585 | 0.4096251 | WFIKKN2 | 37 | 17 |
| cg01101782 | 7.13E−10 | 3.03E−04 | −0.459417978 | 0.459417978 | loss | 0.782747913 | 0.323329935 | KDM58; PCAT6; PCAT6 | 37 | 1 |
| cg23076537 | 7.77E−10 | 3.30E−04 | −0.459125833 | 0.459125833 | loss | 0.826991121 | 0.367865288 | | 37 | 10 |
| cg20631820 | 3.57E−09 | 1.52E−03 | −0.4590784 | 0.4590784 | loss | 0.898103342 | 0.439024941 | | 37 | 13 |
| cg09510752 | 7.13E−10 | 3.03E−04 | −0.459030307 | 0.459030307 | loss | 0.778260313 | 0.319230006 | C5orf66 | 37 | 5 |
| cg02497785 | 9.23E−10 | 3.92E−04 | −0.458802164 | 0.458802164 | loss | 0.819572923 | 0.360770759 | ABCA13 | 37 | 7 |
| cg08247921 | 7.13E−10 | 3.03E−04 | −0.458002921 | 0.458002921 | loss | 0.906608415 | 0.448605494 | MANBA | 37 | 4 |
| cg04638150 | 7.13E−10 | 3.03E−04 | −0.45780909 | 0.45780909 | loss | 0.691707513 | 0.233898424 | AHNAK | 37 | 11 |
| cg08378788 | 7.13E−10 | 3.03E−04 | −0.457665411 | 0.457665411 | loss | 0.70863437 | 0.250968959 | MYO1C; MYO1C; MYO1C | 37 | 17 |
| cg22497095 | 7.13E−10 | 3.03E−04 | −0.457007299 | 0.457007299 | loss | 0.724087664 | 0.267080365 | | 37 | 6 |
| cg13391079 | 7.77E−10 | 3.30E−04 | −0.456887931 | 0.456887931 | loss | 0.868437108 | 0.411549176 | | 37 | 13 |
| cg27576576 | 7.13E−10 | 3.03E−04 | −0.456528327 | 0.456528327 | loss | 0.703864774 | 0.247336447 | | 37 | 7 |
| cg12799314 | 1.19E−09 | 5.07E−04 | −0.456323421 | 0.456323421 | loss | 0.855438526 | 0.399115106 | ADARB2 | 37 | 10 |
| cg11931626 | 7.13E−10 | 3.03E−04 | −0.456005579 | 0.456005579 | loss | 0.669044191 | 0.213038612 | | 37 | 4 |
| cg18206867 | 7.13E−10 | 3.03E−04 | −0.455984926 | 0.455984926 | loss | 0.624660791 | 0.168675865 | B3GNT2 | 37 | 2 |
| cg23327896 | 7.13E−10 | 3.03E−04 | −0.455926801 | 0.455926801 | loss | 0.881712166 | 0.425785365 | DNAJB13 | 37 | 11 |
| cg13591848 | 7.13E−10 | 3.03E−04 | −0.455650127 | 0.455650127 | loss | 0.809051721 | 0.353401594 | PDZD7; PDZD7; SFXN3 | 37 | 10 |
| cg09368716 | 7.13E−10 | 3.03E−04 | −0.455583067 | 0.455583067 | loss | 0.637528302 | 0.181945235 | TMEM88B | 37 | 1 |
| cg24187351 | 7.77E−10 | 3.30E−04 | −0.454776776 | 0.454776776 | loss | 0.790014508 | 0.335237732 | | 37 | X |
| cg07521193 | 7.77E−10 | 3.30E−04 | −0.454746207 | 0.454746207 | loss | 0.950854119 | 0.496107912 | | 37 | 15 |
| cg13105599 | 1.54E−09 | 6.54E−04 | −0.454259544 | 0.454259544 | loss | 0.644306445 | 0.190016901 | | 37 | 15 |
| cg06155303 | 7.13E−10 | 3.03E−04 | −0.454164456 | 0.454164456 | loss | 0.774992526 | 0.320828071 | | 37 | 1 |
| cg26218577 | 7.13E−10 | 3.03E−04 | −0.453835469 | 0.453835469 | loss | 0.561744885 | 0.107909416 | RPLP1; RPLP1 | 37 | 15 |
| cg14433314 | 7.13E−10 | 3.03E−04 | −0.453695126 | 0.453695126 | loss | 0.592780113 | 0.139084988 | | 37 | 7 |
| cg21766667 | 7.13E−10 | 3.03E−04 | −0.453473202 | 0.453473202 | loss | 0.590397979 | 0.136924777 | | 37 | 17 |
| cg05868531 | 7.13E−10 | 3.03E−04 | −0.453332583 | 0.453332583 | loss | 0.497296136 | 0.043963553 | | 37 | 2 |
| cg25337631 | 9.23E−10 | 3.92E−04 | −0.453006988 | 0.453006988 | loss | 0.816117817 | 0.363110829 | | 37 | 10 |
| cg14372753 | 7.13E−10 | 3.03E−04 | −0.452963743 | 0.452963743 | loss | 0.954405702 | 0.501441959 | SLC25A18 | 37 | 22 |
| cg01721149 | 7.13E−10 | 3.03E−04 | −0.452296619 | 0.452296619 | loss | 0.84855516 | 0.396258541 | C1orf61 | 37 | 1 |
| cg14802355 | 7.13E−10 | 3.03E−04 | −0.452110019 | 0.452110019 | loss | 0.892821366 | 0.440711347 | MANBA | 37 | 4 |
| cg13934406 | 7.13E−10 | 3.03E−04 | −0.452039131 | 0.452039131 | loss | 0.903034613 | 0.450995482 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg09965419 | 7.13E−10 | 3.03E−04 | −0.451923319 | 0.451923319 | loss | 0.792788908 | 0.340865588 | DDR1; DDR1; DDR1 | 37 | 6 |
| cg24853438 | 7.13E−10 | 3.03E−04 | −0.451538505 | 0.451538505 | loss | 0.830088975 | 0.378550471 | | 37 | 5 |
| cg12408990 | 7.13E−10 | 3.03E−04 | −0.451391438 | 0.451391438 | loss | 0.658193577 | 0.206802139 | RASGEF1C | 37 | 5 |
| cg19760250 | 7.13E−10 | 3.03E−04 | −0.451310648 | 0.451310648 | loss | 0.882839966 | 0.431529318 | C17orf98; C17orf98 | 37 | 17 |
| cg22952142 | 7.13E−10 | 3.03E−04 | −0.450690172 | 0.450690172 | loss | 0.555406917 | 0.104716745 | | 37 | 15 |
| cg16216407 | 1.01E−09 | 4.27E−04 | −0.450471126 | 0.450471126 | loss | 0.726382849 | 0.275911724 | SOD3 | 37 | 4 |
| cg11905611 | 7.13E−10 | 3.03E−04 | −0.450054007 | 0.450054007 | loss | 0.603572883 | 0.153518876 | PLBD1; PLBD1 | 37 | 12 |
| cg09117688 | 9.23E−10 | 3.92E−04 | −0.449874133 | 0.449874133 | loss | 0.864899209 | 0.415025076 | SUGT1P3; TPTE2P5; TPTE2P5 | 37 | 13 |
| cg18192325 | 7.13E−10 | 3.03E−04 | −0.449553623 | 0.449553623 | loss | 0.836190011 | 0.386636388 | | 37 | 2 |
| cg21252282 | 7.13E−10 | 3.03E−04 | −0.449517086 | 0.449517086 | loss | 0.773240045 | 0.323722959 | | 37 | 9 |
| cg20286956 | 7.13E−10 | 3.03E−04 | −0.449472133 | 0.449472133 | loss | 0.791382304 | 0.341910171 | | 37 | 1 |
| cg03102494 | 9.34E−10 | 3.97E−04 | −0.449436597 | 0.449436597 | loss | 0.549431823 | 0.099995226 | NBPF8; NBPF8; NBPF8 | 37 | 1 |
| cg16915863 | 1.11E−09 | 4.72E−04 | −0.449430754 | 0.449430754 | loss | 0.783428973 | 0.333998219 | LOC400043 | 37 | 12 |
| cg09428340 | 1.01E−09 | 4.27E−04 | −0.44924694 | 0.44924694 | loss | 0.717309604 | 0.268062664 | PACRG; PACRG; PACRG | 37 | 6 |
| cg11557492 | 7.13E−10 | 3.03E−04 | −0.449179151 | 0.449179151 | loss | 0.937042909 | 0.487863759 | SSC5D; SSC5D | 37 | 19 |
| cg20580913 | 7.77E−10 | 3.30E−04 | −0.449024451 | 0.449024451 | loss | 0.922496192 | 0.473471741 | | 37 | 6 |
| cg05226558 | 7.13E−10 | 3.03E−04 | −0.448505343 | 0.448505343 | loss | 0.684416249 | 0.235910906 | TDGF1 | 37 | 3 |
| cg09510531 | 7.13E−10 | 3.03E−04 | −0.448252307 | 0.448252307 | loss | 0.597905604 | 0.149653296 | AIRE | 37 | 21 |
| cg17571919 | 7.13E−10 | 3.03E−04 | −0.448206 | 0.448206 | loss | 0.955981706 | 0.507775706 | LOC401242 | 37 | 6 |
| cg05224659 | 7.13E−10 | 3.03E−04 | −0.448170969 | 0.448170969 | loss | 0.913913128 | 0.465742159 | | 37 | 2 |
| cg22861548 | 1.19E−09 | 5.07E−04 | −0.447999822 | 0.447999822 | loss | 0.710461692 | 0.262461871 | LYNX1; LYNX1; LYNX1; LYNX1; | 37 | 8 |
| | | | | | | | | LYNX1; LYNX1 | | |
| cg11617964 | 7.13E−10 | 3.03E−04 | −0.447735333 | 0.447735333 | loss | 0.621226208 | 0.173490875 | PRRT1 | 37 | 6 |
| cg02430039 | 7.13E−10 | 3.03E−04 | −0.447605425 | 0.447605425 | loss | 0.658096355 | 0.210490929 | | 37 | 17 |
| cg14759977 | 7.13E−10 | 3.03E−04 | −0.447396014 | 0.447396014 | loss | 0.840101455 | 0.392705441 | SUGT1P3; TPTE2P5; TPTE2P5 | 37 | 13 |
| cg21182196 | 7.13E−10 | 3.03E−04 | −0.447255761 | 0.447255761 | loss | 0.875447455 | 0.428191694 | CLMN | 37 | 14 |
| cg03188580 | 7.84E−10 | 3.33E−04 | −0.44722037 | 0.44722037 | loss | 0.952674517 | 0.505454147 | LOC401242 | 37 | 6 |
| cg19389372 | 7.13E−10 | 3.03E−04 | −0.447030533 | 0.447030533 | loss | 0.511750126 | 0.064719593 | SDHAF1 | 37 | 19 |
| cg08123207 | 1.89E−09 | 8.01E−04 | −0.446981133 | 0.446981133 | loss | 0.573939827 | 0.126958694 | PRRT1 | 37 | 6 |
| cg15584071 | 7.13E−10 | 3.03E−04 | −0.446542535 | 0.446542535 | loss | 0.594724653 | 0.148182118 | SDHAF1 | 37 | 19 |
| cg14459158 | 7.13E−10 | 3.03E−04 | −0.446480756 | 0.446480756 | loss | 0.576235943 | 0.129755187 | | 37 | 9 |
| cg19579217 | 7.13E−10 | 3.03E−04 | −0.446452477 | 0.446452477 | loss | 0.580129683 | 0.133677206 | | 37 | 6 |
| cg08877188 | 7.13E−10 | 3.03E−04 | −0.446256795 | 0.446256795 | loss | 0.809929625 | 0.363672829 | SLC51A | 37 | 3 |
| cg22109795 | 9.54E−09 | 4.05E−03 | −0.445996626 | 0.445996626 | loss | 0.859000096 | 0.413003471 | | 37 | 1 |
| cg03042692 | 7.13E−10 | 3.03E−04 | −0.445911837 | 0.445911837 | loss | 0.752814866 | 0.306903029 | | 37 | 13 |
| cg25661792 | 7.13E−10 | 3.03E−04 | −0.445616592 | 0.445616592 | loss | 0.746268304 | 0.300651712 | | 37 | 3 |
| cg19328828 | 2.86E−09 | 1.22E−03 | −0.445423347 | 0.445423347 | loss | 0.867949012 | 0.422525665 | HCAR1 | 37 | 12 |
| cg04579398 | 7.13E−10 | 3.03E−04 | −0.445236975 | 0.445236975 | loss | 0.4972316 | 0.051994625 | SDHAF1 | 37 | 19 |
| cg00611789 | 8.56E−10 | 3.63E−04 | −0.444845076 | 0.444845076 | loss | 0.744315412 | 0.299470336 | CMYA5 | 37 | 5 |
| cg25460807 | 8.47E−10 | 3.60E−04 | −0.444522984 | 0.444522984 | loss | 0.765672355 | 0.321149371 | | 37 | 8 |
| cg07266144 | 7.13E−10 | 3.03E−04 | −0.444505758 | 0.444505758 | loss | 0.629984774 | 0.185479015 | PDLIM4; PDLIM4 | 37 | 5 |
| cg17378342 | 7.13E−10 | 3.03E−04 | −0.44413315 | 0.44413315 | loss | 0.870191762 | 0.426058612 | CDX1 | 37 | 5 |
| cg06092502 | 1.68E−09 | 7.12E−04 | −0.444090375 | 0.444090375 | loss | 0.755322775 | 0.3112324 | | 37 | 1 |
| cg19962075 | 9.23E−10 | 3.92E−04 | −0.443973314 | 0.443973314 | loss | 0.753733455 | 0.309760141 | MAPK4 | 37 | 18 |
| cg10831246 | 7.13E−10 | 3.03E−04 | −0.443951415 | 0.443951415 | loss | 0.693290274 | 0.249338859 | | 37 | 17 |
| cg02247178 | 7.13E−10 | 3.03E−04 | −0.443863653 | 0.443863653 | loss | 0.720639553 | 0.2767759 | TEX26; TEX26-AS1; TEX26- | 37 | 13 |
| | | | | | | | | AS1; TEX26-AS1; TEX26- | | |
| | | | | | | | | AS1 | | |
| cg01206378 | 7.84E−10 | 3.33E−04 | −0.443754379 | 0.443754379 | loss | 0.791688256 | 0.347933876 | RWDD3; RWDD3; TMEM56- | 37 | 1 |
| | | | | | | | | RWDD3; RWDD3; RWDD3; | | |
| | | | | | | | | RWDD3; RWDD3; RWDD3 | | |
| cg03466525 | 7.13E−10 | 3.03E−04 | −0.443625613 | 0.443625613 | loss | 0.550956379 | 0.107330766 | | 37 | 10 |
| cg27438152 | 6.35E−09 | 2.70E−03 | −0.443192915 | 0.443192915 | loss | 0.837630974 | 0.394438059 | LOC283710 | 37 | 15 |
| cg10244666 | 7.13E−10 | 3.03E−04 | −0.442677943 | 0.442677943 | loss | 0.975410802 | 0.532732859 | WT1; WT1; WT1; WT1; WT1 | 37 | 11 |
| cg12914966 | 7.13E−10 | 3.03E−04 | −0.442568959 | 0.442568959 | loss | 0.902508536 | 0.459939576 | LOC401242 | 37 | 6 |
| cg17445765 | 1.42E−09 | 6.01E−04 | −0.442562429 | 0.442562429 | loss | 0.706756692 | 0.264194264 | | 37 | 7 |
| cg06025456 | 7.13E−10 | 3.03E−04 | −0.441690502 | 0.441690502 | loss | 0.913417149 | 0.471726647 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg24010336 | 7.13E−10 | 3.03E−04 | −0.441409519 | 0.441409519 | loss | 0.765213508 | 0.323803988 | FBXO17; FBXO17 | 37 | 19 |
| cg20909380 | 7.13E−10 | 3.03E−04 | −0.441399425 | 0.441399425 | loss | 0.880811719 | 0.439412294 | | 37 | 15 |
| cg25483741 | 7.13E−10 | 3.03E−04 | −0.441384981 | 0.441384981 | loss | 0.802173428 | 0.360788447 | LYNX1; LYNX1; LYNX1; LYNX1 | 37 | 8 |
| cg08979191 | 2.16E−09 | 9.17E−04 | −0.441355199 | 0.441355199 | loss | 0.747086123 | 0.305730924 | SEPT8; SEPT8; SEPT8; SEPT8; | 37 | 5 |
| | | | | | | | | SEPT8 | | |
| cg12236088 | 7.77E−10 | 3.30E−04 | −0.441264269 | 0.441264269 | loss | 0.534429026 | 0.093164757 | | 37 | 2 |
| cg08216684 | 7.13E−10 | 3.03E−04 | −0.441140773 | 0.441140773 | loss | 0.880648232 | 0.439507459 | | 37 | 5 |
| cg24070990 | 1.01E−09 | 4.27E−04 | −0.441018401 | 0.441018401 | loss | 0.577719291 | 0.136700889 | | 37 | 1 |
| cg12974138 | 7.13E−10 | 3.03E−04 | −0.440904275 | 0.440904275 | loss | 0.555309711 | 0.114405436 | | 37 | 11 |
| cg11669516 | 7.13E−10 | 3.03E−04 | −0.440664324 | 0.440664324 | loss | 0.759324689 | 0.318660365 | IGFBP2 | 37 | 2 |
| cg09655403 | 9.23E−10 | 3.92E−04 | −0.440607506 | 0.440607506 | loss | 0.810391789 | 0.369784283 | CMYA5 | 37 | 5 |
| cg19978312 | 7.13E−10 | 3.03E−04 | −0.440570624 | 0.440570624 | loss | 0.688345583 | 0.247774959 | RASGEF1C | 37 | 5 |
| cg21475076 | 7.13E−10 | 3.03E−04 | −0.440253881 | 0.440253881 | loss | 0.96160654 | 0.521352659 | | 37 | 1 |
| cg05819154 | 7.13E−10 | 3.03E−04 | −0.439695857 | 0.439695857 | loss | 0.957510787 | 0.517814929 | | 37 | 16 |
| cg02452552 | 7.13E−10 | 3.03E−04 | −0.439337408 | 0.439337408 | loss | 0.761259396 | 0.321921988 | | 37 | 5 |
| cg00083206 | 7.13E−10 | 3.03E−04 | −0.439036577 | 0.439036577 | loss | 0.62387423 | 0.184837653 | DDO; DDO | 37 | 6 |
| cg07429623 | 4.21E−09 | 1.79E−03 | −0.438711523 | 0.438711523 | loss | 0.729110364 | 0.290398841 | KLHDC8A; KLHDC8A; KLHDC8A; | 37 | 1 |
| | | | | | | | | KLHDC8A; KLHDC8A; | | |
| | | | | | | | | KLHDC8A; KLHDC8A; KLHDC8A | | |
| cg10663765 | 7.13E−10 | 3.03E−04 | −0.438614247 | 0.438614247 | loss | 0.659999794 | 0.221385547 | | 37 | 3 |
| cg17330303 | 7.13E−10 | 3.03E−04 | −0.437921358 | 0.437921358 | loss | 0.980371523 | 0.542450165 | SFN | 37 | 1 |
| cg16306978 | 7.77E−10 | 3.30E−04 | −0.437410271 | 0.437410271 | loss | 0.521764387 | 0.084354115 | APOB; APOB | 37 | 2 |
| cg03679504 | 7.13E−10 | 3.03E−04 | −0.437376432 | 0.437376432 | loss | 0.924294096 | 0.486917665 | TDGF1; TDGF1 | 37 | 3 |
| cg08289130 | 7.13E−10 | 3.03E−04 | −0.437251558 | 0.437251558 | loss | 0.781742281 | 0.344490724 | | 37 | 16 |
| cg23180489 | 7.13E−10 | 3.03E−04 | −0.43724148 | 0.43724148 | loss | 0.745301604 | 0.308060124 | LYNX1; LYNX1; LYNX1; LYNX1; | 37 | 8 |
| | | | | | | | | LYNX1; LYNX1 | | |
| cg02783887 | 7.77E−10 | 3.30E−04 | −0.437091664 | 0.437091664 | loss | 0.877204823 | 0.440113159 | | 37 | 2 |
| cg13392078 | 7.13E−10 | 3.03E−04 | −0.437025495 | 0.437025495 | loss | 0.529964617 | 0.092939122 | RECK | 37 | 9 |
| cg17662083 | 7.13E−10 | 3.03E−04 | −0.436885738 | 0.436885738 | loss | 0.790162326 | 0.353276588 | DMKN; DMKN; DMKN; DMKN; | 37 | 19 |
| | | | | | | | | DMKN; DMKN; DMKN; | | |
| | | | | | | | | DMKN; DMKN; DMKN; DMKN; | | |
| | | | | | | | | DMKN; DMKN; DMKN | | |
| cg06878548 | 8.47E−10 | 3.60E−04 | −0.436724094 | 0.436724094 | loss | 0.849206953 | 0.412482859 | | 37 | 8 |
| cg24808901 | 7.13E−10 | 3.03E−04 | −0.436615518 | 0.436615518 | loss | 0.680602836 | 0.243987318 | RWDD3; RWDD3; TMEM56- | 37 | 1 |
| | | | | | | | | RWDD3; RWDD3; RWDD3; | | |
| | | | | | | | | RWDD3; RWDD3; RWDD3 | | |
| cg25453677 | 7.13E−10 | 3.03E−04 | −0.436426017 | 0.436426017 | loss | 0.915532223 | 0.479106206 | | 37 | 17 |
| cg08089567 | 7.84E−10 | 3.33E−04 | −0.436395835 | 0.436395835 | loss | 0.601636587 | 0.165240751 | AIRE | 37 | 21 |
| cg07853425 | 7.13E−10 | 3.03E−04 | −0.435434661 | 0.435434661 | loss | 0.782144702 | 0.346710041 | C5orf66 | 37 | 5 |
| cg20592017 | 8.47E−10 | 3.60E−04 | −0.435313097 | 0.435313097 | loss | 0.757687715 | 0.322374618 | | 37 | 15 |
| cg12231340 | 7.13E−10 | 3.03E−04 | −0.434705527 | 0.434705527 | loss | 0.800103809 | 0.365398282 | KRT81; KRT81 | 37 | 12 |
| cg18361418 | 7.13E−10 | 3.03E−04 | −0.434619279 | 0.434619279 | loss | 0.772058632 | 0.337439353 | GPX5; GPX5 | 37 | 6 |
| cg17493885 | 7.13E−10 | 3.03E−04 | −0.434571943 | 0.434571943 | loss | 0.513669457 | 0.079097514 | NSD1; NSD1 | 37 | 5 |
| cg19264571 | 7.13E−10 | 3.03E−04 | −0.43445197 | 0.43445197 | loss | 0.830105958 | 0.395653988 | APCDD1 | 37 | 18 |
| cg23279355 | 8.47E−10 | 3.60E−04 | −0.433968063 | 0.433968063 | loss | 0.744671651 | 0.310703588 | CMYA5 | 37 | 5 |
| cg14003022 | 7.13E−10 | 3.03E−04 | −0.433889179 | 0.433889179 | loss | 0.557603534 | 0.123714355 | | 37 | 4 |
| cg01791034 | 7.13E−10 | 3.03E−04 | −0.433756266 | 0.433756266 | loss | 0.892548772 | 0.458792506 | PLA2G4E | 37 | 15 |
| cg22455128 | 7.13E−10 | 3.03E−04 | −0.433672053 | 0.433672053 | loss | 0.845758706 | 0.412086653 | | 37 | 13 |
| cg25665606 | 7.13E−10 | 3.03E−04 | −0.433597248 | 0.433597248 | loss | 0.819543436 | 0.385946188 | KLHDC7B | 37 | 22 |
| cg19227031 | 7.13E−10 | 3.03E−04 | −0.433536644 | 0.433536644 | loss | 0.635643751 | 0.202107106 | PRRT1 | 37 | 6 |
| cg03453449 | 7.13E−10 | 3.03E−04 | −0.433524286 | 0.433524286 | loss | 0.831242809 | 0.397718524 | USP44; USP44 | 37 | 12 |
| cg16484473 | 7.77E−10 | 3.30E−04 | −0.433460241 | 0.433460241 | loss | 0.570568781 | 0.13710854 | | 37 | 10 |
| cg20632887 | 7.13E−10 | 3.03E−04 | −0.433443052 | 0.433443052 | loss | 0.744937134 | 0.311494082 | ATP1A2 | 37 | 1 |
| cg00399059 | 8.47E−10 | 3.60E−04 | −0.433437001 | 0.433437001 | loss | 0.692568866 | 0.259131865 | LYNX1; LYNX1; LYNX1; LYNX1; | 37 | 8 |
| | | | | | | | | LYNX1; LYNX1 | | |
| cg07168556 | 7.13E−10 | 3.03E−04 | −0.433421679 | 0.433421679 | loss | 0.780553326 | 0.347131647 | WFIKKN2 | 37 | 17 |
| cg21277452 | 7.13E−10 | 3.03E−04 | −0.433329675 | 0.433329675 | loss | 0.899778034 | 0.466448359 | LOC401242 | 37 | 6 |
| cg27179622 | 7.13E−10 | 3.03E−04 | −0.433156512 | 0.433156512 | loss | 0.483910681 | 0.050754169 | LEFTY2; LEFTY2 | 37 | 1 |
| cg00070899 | 7.13E−10 | 3.03E−04 | −0.43278351 | 0.43278351 | loss | 0.871877634 | 0.439094124 | GRM4; GRM4; GRM4; GRM4; | 37 | 6 |
| | | | | | | | | GRM4; GRM4 | | |
| cg13058819 | 7.77E−10 | 3.30E−04 | −0.432737448 | 0.432737448 | loss | 0.714937591 | 0.282200143 | SLC35F3 | 37 | 1 |
| cg12407057 | 7.13E−10 | 3.03E−04 | −0.432459074 | 0.432459074 | loss | 0.744015409 | 0.311556336 | | 37 | 1 |
| cg07752120 | 3.14E−08 | 1.33E−02 | −0.432319168 | 0.432319168 | loss | 0.728080504 | 0.295761335 | | 37 | 5 |
| cg09013220 | 7.12E−10 | 3.02E−04 | −0.43229697 | 0.43229697 | loss | 0.975596164 | 0.543299194 | | 37 | 17 |
| cg10859192 | 7.13E−10 | 3.03E−04 | −0.432120959 | 0.432120959 | loss | 0.841409836 | 0.409288876 | ATP5G2 | 37 | 12 |
| cg19786602 | 3.57E−09 | 1.52E−03 | −0.431617292 | 0.431617292 | loss | 0.561260043 | 0.129642751 | | 37 | 17 |
| cg17910899 | 7.77E−10 | 3.30E−04 | −0.431510981 | 0.431510981 | loss | 0.699586489 | 0.268075508 | | 37 | 13 |
| cg21334513 | 7.48E−09 | 3.18E−03 | −0.431064817 | 0.431064817 | loss | 0.518203083 | 0.087138266 | | 37 | 6 |
| cg12716639 | 7.13E−10 | 3.03E−04 | −0.430952813 | 0.430952813 | loss | 0.62302554 | 0.192072726 | USP2; USP2; USP2 | 37 | 11 |
| cg24024660 | 7.13E−10 | 3.03E−04 | −0.43088645 | 0.43088645 | loss | 0.650679332 | 0.219792882 | LOC100289187; LOC100289187; | 37 | 7 |
| | | | | | | | | LOC100289187 | | |
| cg16361867 | 7.13E−10 | 3.03E−04 | −0.430760454 | 0.430760454 | loss | 0.786018025 | 0.355257571 | GRIK4; GRIK4; GRIK4 | 37 | 11 |
| cg12182937 | 7.13E−10 | 3.03E−04 | −0.430719345 | 0.430719345 | loss | 0.610199898 | 0.179480553 | | 37 | 6 |
| cg14906690 | 7.13E−10 | 3.03E−04 | −0.430642438 | 0.430542438 | loss | 0.847776621 | 0.417134182 | | 37 | 10 |
| cg00213295 | 9.23E−10 | 3.92E−04 | −0.430574566 | 0.430574566 | loss | 0.767620525 | 0.337045959 | TMEM132E | 37 | 17 |
| cg06687305 | 7.13E−10 | 3.03E−04 | −0.430532675 | 0.430532675 | loss | 0.785018687 | 0.354486012 | CCDC17; CCDC17 | 37 | 1 |
| cg01119278 | 7.13E−10 | 3.03E−04 | −0.430435214 | 0.430435214 | loss | 0.619039338 | 0.188604124 | DDO; DDO | 37 | 6 |
| cg25356715 | 7.13E−10 | 3.03E−04 | −0.430152567 | 0.430152567 | loss | 0.904308791 | 0.474156224 | CACNA2D4 | 37 | 12 |
| cg24874173 | 1.42E−09 | 6.01E−04 | −0.430047679 | 0.430047679 | loss | 0.881110595 | 0.451062918 | NBPF10; LOC100288142 | 37 | 1 |
| cg00107241 | 7.13E−10 | 3.03E−04 | −0.42978486 | 0.42978486 | loss | 0.760530672 | 0.330745812 | | 37 | 1 |
| cg16677448 | 7.13E−10 | 3.03E−04 | −0.42973487 | 0.42973487 | loss | 0.593930164 | 0.164195294 | DMKN; DMKN; DMKN; DMKN; | 37 | 19 |
| | | | | | | | | DMKN; DMKN; DMKN; | | |
| | | | | | | | | DMKN; DMKN; DMKN; DMKN; | | |
| | | | | | | | | DMKN; DMKN; DMKN | | |
| cg04388919 | 7.13E−10 | 3.03E−04 | −0.429588264 | 0.429588264 | loss | 0.694471417 | 0.264883153 | | 37 | 7 |
| cg05745631 | 2.68E−09 | 1.14E−03 | −0.429281021 | 0.429281021 | loss | 0.726177621 | 0.2968966 | | 37 | 6 |
| cg02703145 | 7.13E−10 | 3.03E−04 | −0.429137915 | 0.429137915 | loss | 0.854636315 | 0.4254984 | | 37 | 9 |
| cg16087940 | 7.13E−10 | 3.03E−04 | −0.429052447 | 0.429052447 | loss | 0.807983658 | 0.378931212 | | 37 | 10 |
| cg00481725 | 7.13E−10 | 3.03E−04 | −0.428828307 | 0.428828307 | loss | 0.778284813 | 0.349456506 | | 37 | 15 |
| cg12033622 | 2.06E−09 | 8.75E−04 | −0.42868496 | 0.42868496 | loss | 0.670741028 | 0.242056069 | COX4I2 | 37 | 20 |
| cg18228590 | 1.10E−09 | 4.65E−04 | −0.428633015 | 0.428633015 | loss | 0.706522015 | 0.277889 | | 37 | 5 |
| cg18700133 | 7.13E−10 | 3.03E−04 | −0.427883518 | 0.427883518 | loss | 0.89839113 | 0.470507612 | ALOXE3; ALOXE3 | 37 | 17 |
| cg25642234 | 7.13E−10 | 3.03E−04 | −0.427643568 | 0.427643568 | loss | 0.649396468 | 0.2217529 | PLBD1 | 37 | 12 |
| cg15920791 | 7.13E−10 | 3.03E−04 | −0.427632047 | 0.427632047 | loss | 0.577747506 | 0.150115458 | | 37 | 8 |
| cg13641713 | 7.77E−10 | 3.30E−04 | −0.4274861 | 0.4274861 | loss | 0.761073247 | 0.333587147 | | 37 | 9 |
| cg13734833 | 7.13E−10 | 3.03E−04 | −0.427444979 | 0.427444979 | loss | 0.863288679 | 0.4358437 | MRGPRF; MRGPRF | 37 | 11 |
| cg03593471 | 7.13E−10 | 3.03E−04 | −0.427398133 | 0.427398133 | loss | 0.631692857 | 0.204294724 | | 37 | 8 |
| cg20889395 | 7.13E−10 | 3.03E−04 | −0.427392318 | 0.427392318 | loss | 0.877910889 | 0.450518571 | WFIKKN2 | 37 | 17 |
| cg12127196 | 7.13E−10 | 3.03E−04 | −0.427257882 | 0.427257882 | loss | 0.659903711 | 0.232645829 | AGBL4 | 37 | 1 |
| cg21215576 | 7.13E−10 | 3.03E−04 | −0.427107581 | 0.427107581 | loss | 0.777901875 | 0.350794294 | FAM83A; FAM83A | 37 | 8 |
| cg21386414 | 7.13E−10 | 3.03E−04 | −0.426953145 | 0.426953145 | loss | 0.760909692 | 0.333956547 | | 37 | 1 |
| cg04459939 | 7.13E−10 | 3.03E−04 | −0.426785848 | 0.426785848 | loss | 0.738748366 | 0.311962518 | | 37 | 15 |
| cg07191900 | 7.13E−10 | 3.03E−04 | −0.426687825 | 0.426687825 | loss | 0.956863219 | 0.530175394 | IL17RA | 37 | 22 |
| cg13842639 | 7.13E−10 | 3.03E−04 | −0.426448774 | 0.426448774 | loss | 0.753282621 | 0.326833847 | | 37 | 9 |
| cg07734975 | 7.12E−10 | 3.02E−04 | −0.426410518 | 0.426410518 | loss | 0.66808094 | 0.241670421 | LOC440028; SBF2-AS1 | 37 | 11 |
| cg24002541 | 7.77E−10 | 3.30E−04 | −0.426218692 | 0.426218692 | loss | 0.564264617 | 0.138045925 | | 37 | 10 |
| cg14552982 | 7.13E−10 | 3.03E−04 | −0.426213792 | 0.426213792 | loss | 0.689216815 | 0.263003024 | EXOSC2; EXOSC2; EXOSC2; | 37 | 9 |
| | | | | | | | | EXOSC2 | | |
| cg01395127 | 7.13E−10 | 3.03E−04 | −0.426093218 | 0.426093218 | loss | 0.733783894 | 0.307690676 | | 37 | 6 |
| cg13202523 | 7.13E−10 | 3.03E−04 | −0.425775578 | 0.425775578 | loss | 0.794403755 | 0.368628176 | | 37 | 7 |
| cg04715503 | 7.13E−10 | 3.03E−04 | −0.425647976 | 0.425647976 | loss | 0.459286749 | 0.033638773 | CLDN9 | 37 | 16 |
| cg08880849 | 7.13E−10 | 3.03E−04 | −0.425467961 | 0.425467961 | loss | 0.953452049 | 0.527984088 | KAT2A; HSPB9 | 37 | 17 |
| cg20245362 | 8.47E−10 | 3.60E−04 | −0.425422992 | 0.425422992 | loss | 0.588146117 | 0.152723125 | | 37 | 7 |
| cg09373015 | 7.13E−10 | 3.03E−04 | −0.424966002 | 0.424966002 | loss | 0.78421356 | 0.359247559 | | 37 | 6 |
| cg13702536 | 1.68E−09 | 7.12E−04 | −0.424889602 | 0.424889602 | loss | 0.896453291 | 0.471563688 | HCAR1 | 37 | 12 |
| cg22670033 | 7.13E−10 | 3.03E−04 | −0.42481428 | 0.42481428 | loss | 0.801083121 | 0.376268841 | | 37 | 5 |
| cg03342928 | 7.13E−10 | 3.03E−04 | −0.424245177 | 0.424245177 | loss | 0.951381647 | 0.527136471 | SSC5D; SSC5D | 37 | 19 |
| cg06489965 | 7.13E−10 | 3.03E−04 | −0.424209384 | 0.424209384 | loss | 0.574647392 | 0.150438008 | CLDN9 | 37 | 16 |
| cg27268279 | 7.13E−10 | 3.03E−04 | −0.424071781 | 0.424071781 | loss | 0.87342504 | 0.449353259 | COL13A1; COL13A1; COL13A1; | 37 | 10 |
| | | | | | | | | COL13A1; COL13A1; COL13A1 | | |
| cg27532360 | 7.13E−10 | 3.03E−04 | −0.424029278 | 0.424029278 | loss | 0.716325272 | 0.292295994 | ITGA2B | 37 | 17 |
| cg09814029 | 7.77E−10 | 3.30E−04 | −0.423970318 | 0.423970318 | loss | 0.793402736 | 0.369432418 | | 37 | 1 |
| cg02723291 | 7.13E−10 | 3.03E−04 | −0.423569806 | 0.423569806 | loss | 0.920354247 | 0.496784441 | ETNK1; ETNK1 | 37 | 12 |
| cg05293490 | 7.13E−10 | 3.03E−04 | −0.423135475 | 0.423135475 | loss | 0.577749336 | 0.154613861 | | 37 | 14 |
| cg03532263 | 7.13E−10 | 3.03E−04 | −0.422895288 | 0.422895288 | loss | 0.865927606 | 0.443032318 | | 37 | 14 |
| cg07349815 | 7.77E−10 | 3.30E−04 | −0.422804211 | 0.422804211 | loss | 0.868074128 | 0.445269918 | | 37 | 3 |
| cg24460126 | 8.47E−10 | 3.60E−04 | −0.422774168 | 0.422774168 | loss | 0.742940862 | 0.320166695 | FAM163A | 37 | 1 |
| cg14481208 | 7.13E−10 | 3.03E−04 | −0.422711739 | 0.422711739 | loss | 0.498578643 | 0.075866904 | RTKN | 37 | 2 |
| cg06555959 | 7.13E−10 | 3.03E−04 | −0.422649053 | 0.422649053 | loss | 0.680639436 | 0.257990382 | | 37 | 8 |
| cg12529273 | 7.13E−10 | 3.03E−04 | −0.422625629 | 0.422625629 | loss | 0.742915123 | 0.320289494 | LNX1; LNX1-AS2 | 37 | 4 |
| cg02179982 | 7.13E−10 | 3.03E−04 | −0.42250799 | 0.42250799 | loss | 0.664576849 | 0.242068859 | WNT7B | 37 | 22 |
| cg26776957 | 7.13E−10 | 3.03E−04 | −0.422486715 | 0.422486715 | loss | 0.811666268 | 0.389179553 | PLEKHB1; PLEKHB1; PLEKHB1; | 37 | 11 |
| | | | | | | | | PLEKHB1 | | |
| cg05912299 | 7.13E−10 | 3.03E−04 | −0.422437845 | 0.422437845 | loss | 0.701352945 | 0.2789151 | | 37 | 13 |
| cg12094552 | 7.13E−10 | 3.03E−04 | −0.422301786 | 0.422301786 | loss | 0.802543609 | 0.380241824 | | 37 | 21 |
| cg14947478 | 1.21E−09 | 5.15E−04 | −0.422193486 | 0.422193486 | loss | 0.804206592 | 0.382013106 | GDF5; GDF5 | 37 | 20 |
| cg01207473 | 7.13E−10 | 3.03E−04 | −0.421932638 | 0.421932638 | loss | 0.623923721 | 0.201991082 | | 37 | 6 |
| cg14172108 | 7.13E−10 | 3.03E−04 | −0.421812727 | 0.421812727 | loss | 0.502929838 | 0.081117111 | | 37 | 21 |
| cg21109038 | 1.73E−09 | 7.34E−04 | −0.421754025 | 0.421754025 | loss | 0.968512981 | 0.546758956 | ZNF710 | 37 | 15 |
| cg01510388 | 7.13E−10 | 3.03E−04 | −0.421730914 | 0.421730914 | loss | 0.481454606 | 0.059723692 | CYP7B1 | 37 | 8 |
| cg23193613 | 1.42E−09 | 6.01E−04 | −0.421511596 | 0.421511596 | loss | 0.765657996 | 0.3441464 | | 37 | 8 |
| cg13411784 | 7.13E−10 | 3.03E−04 | −0.42143053 | 0.42143053 | loss | 0.545832215 | 0.124401685 | DPH7 | 37 | 9 |
| cg03677069 | 7.13E−10 | 3.03E−04 | −0.421274657 | 0.421274657 | loss | 0.700661734 | 0.279387076 | SNCG; MMRN2 | 37 | 10 |
| cg04896168 | 8.47E−10 | 3.60E−04 | −0.421232698 | 0.421232698 | loss | 0.666034104 | 0.244801406 | | 37 | 1 |
| cg11069430 | 7.13E−10 | 3.03E−04 | −0.421214562 | 0.421214562 | loss | 0.846837174 | 0.425622612 | USP44; USP44 | 37 | 12 |
| cg01343363 | 9.23E−10 | 3.92E−04 | −0.42112461 | 0.42112461 | loss | 0.614312534 | 0.193187924 | NDRG4; NDRG4; NDRG4; NDRG4; | 37 | 16 |
| | | | | | | | | NDRG4; NDRG4; NDRG4; | | |
| | | | | | | | | NDRG4 | | |
| cg12229151 | 7.13E−10 | 3.03E−04 | −0.42050819 | 0.42050819 | loss | 0.920107308 | 0.499599118 | PRKCDBP | 37 | 11 |
| cg20481312 | 7.13E−10 | 3.03E−04 | −0.420199988 | 0.420199988 | loss | 0.588064317 | 0.167864329 | | 37 | 15 |
| cg09047034 | 7.13E−10 | 3.03E−04 | −0.420189059 | 0.420189059 | loss | 0.869142706 | 0.448953647 | | 37 | 2 |
| cg04821248 | 7.77E−10 | 3.30E−04 | −0.41989424 | 0.41989424 | loss | 0.745846223 | 0.325951982 | | 37 | 16 |
| cg13266496 | 7.13E−10 | 3.03E−04 | −0.41977953 | 0.41977953 | loss | 0.595713358 | 0.175933828 | DDO; DDO | 37 | 6 |
| cg01607625 | 1.89E−09 | 8.02E−04 | −0.419030129 | 0.419030129 | loss | 0.657844719 | 0.238814589 | | 37 | 6 |
| cg01863042 | 7.13E−10 | 3.03E−04 | −0.418605245 | 0.418605245 | loss | 0.577892743 | 0.159287498 | | 37 | 13 |
| cg08610426 | 7.84E−10 | 3.33E−04 | −0.418586638 | 0.418586638 | loss | 0.503075687 | 0.084489048 | IZUMO1 | 37 | 19 |
| cg20595752 | 8.47E−10 | 3.60E−04 | −0.418470647 | 0.418470647 | loss | 0.61069483 | 0.192224184 | TEAD3 | 37 | 6 |
| cg13349472 | 7.84E−10 | 3.33E−04 | −0.418103514 | 0.418103514 | loss | 0.610917802 | 0.192814288 | | 37 | 2 |
| cg21687003 | 9.23E−10 | 3.92E−04 | −0.418089801 | 0.418089801 | loss | 0.83794793 | 0.419858129 | | 37 | 4 |
| cg10541110 | 7.13E−10 | 3.03E−04 | −0.418009293 | 0.418009293 | loss | 0.637335258 | 0.219325966 | KRTAP2-3 | 37 | 17 |
| cg11210878 | 7.13E−10 | 3.03E−04 | −0.417974219 | 0.417974219 | loss | 0.859103813 | 0.441129594 | CHRM1; CHRM1 | 37 | 11 |
| cg01293485 | 7.13E−10 | 3.03E−04 | −0.417782969 | 0.417782969 | loss | 0.73833454 | 0.320551571 | HIGD1A; HIGD1A; HIGD1A | 37 | 3 |
| cg18927077 | 7.13E−10 | 3.03E−04 | −0.41750288 | 0.41750288 | loss | 0.570423851 | 0.152920971 | | 37 | 14 |
| cg24579589 | 7.13E−10 | 3.03E−04 | −0.417458722 | 0.417458722 | loss | 0.785515234 | 0.368056512 | | 37 | 1 |
| cg06672093 | 1.30E−09 | 5.52E−04 | −0.417314418 | 0.417314418 | loss | 0.738766343 | 0.321451926 | | 37 | X |
| cg12733396 | 7.13E−10 | 3.03E−04 | −0.41729834 | 0.41729834 | loss | 0.706159368 | 0.288861028 | COL9A2 | 37 | 1 |
| cg19860353 | 7.77E−10 | 3.30E−04 | −0.417187853 | 0.417187853 | loss | 0.876217006 | 0.459029153 | | 37 | 3 |
| cg18890544 | 7.13E−10 | 3.03E−04 | 0.417053969 | 0.417053969 | loss | 0.900906651 | 0.483852682 | | 37 | 1 |
| cg18959621 | 7.13E−10 | 3.03E−04 | −0.417048971 | 0.417048971 | loss | 0.735294489 | 0.318245518 | | 37 | 13 |
| cg25240254 | 7.13E−10 | 3.03E−04 | −0.417042314 | 0.417042314 | loss | 0.649139896 | 0.232097582 | SSC5D; SSC5D | 37 | 19 |
| cg26884773 | 7.13E−10 | 3.03E−04 | −0.416819931 | 0.416819931 | loss | 0.832420972 | 0.415601041 | | 37 | 1 |
| cg09938479 | 7.13E−10 | 3.03E−04 | −0.416766921 | 0.416766921 | loss | 0.537001615 | 0.120234694 | | 37 | 1 |
| cg15602677 | 1.02E−09 | 4.33E−04 | −0.41666814 | 0.41666814 | loss | 0.808034435 | 0.391366294 | SNTG2 | 37 | 2 |
| cg19127638 | 7.13E−10 | 3.03E−04 | −0.416471092 | 0.416471092 | loss | 0.827089115 | 0.410618024 | TET1 | 37 | 10 |
| cg07824824 | 7.13E−10 | 3.03E−04 | −0.416376538 | 0.416376538 | loss | 0.547124238 | 0.1307477 | CIDEA; CIDEA | 37 | 18 |
| cg22031964 | 7.13E−10 | 3.03E−04 | −0.416078236 | 0.416078236 | loss | 0.953505636 | 0.5374274 | CHRM1 | 37 | 11 |
| cg13647973 | 8.47E−10 | 3.60E−04 | −0.415989289 | 0.415989289 | loss | 0.896632343 | 0.480643054 | | 37 | 19 |
| cg02904657 | 7.13E−10 | 3.03E−04 | −0.415554739 | 0.415554739 | loss | 0.664038768 | 0.248484029 | | 37 | 15 |
| cg08274637 | 7.13E−10 | 3.03E−04 | −0.41539797 | 0.41539797 | loss | 0.752182175 | 0.336784206 | DLEU7; DLEU7; DLEU7- | 37 | 13 |
| | | | | | | | | AS1 | | |
| cg09616559 | 5.41E−08 | 2.30E−02 | −0.415318245 | 0.415318245 | loss | 0.654129462 | 0.238811218 | | 37 | 2 |
| cg01887374 | 7.13E−10 | 3.03E−04 | −0.415292823 | 0.415292823 | loss | 0.708072217 | 0.292779394 | HS6ST1 | 37 | 2 |
| cg02299497 | 7.13E−10 | 3.03E−04 | −0.415265345 | 0.415265345 | loss | 0.447015116 | 0.031749771 | | 37 | 14 |
| cg14625636 | 1.01E−09 | 4.27E−04 | −0.415256893 | 0.415256893 | loss | 0.79291164 | 0.377654747 | LOC100499484; LOC100499484- | 37 | 9 |
| | | | | | | | | C9ORF174; LOC100499484- | | |
| | | | | | | | | C9ORF174; LOC100499484- | | |
| | | | | | | | | C9ORF174 | | |
| cg03993109 | 7.13E−10 | 3.03E−04 | −0.41505912 | 0.41505912 | loss | 0.856502796 | 0.441443676 | ADAMTS2; ADAMTS2 | 37 | 5 |
| cg09221960 | 7.13E−10 | 3.03E−04 | −0.415029029 | 0.415029029 | loss | 0.9309336 | 0.515904571 | GRIK4; GRIK4; GRIK4 | 37 | 11 |
| cg01043616 | 7.13E−10 | 3.03E−04 | −0.414424433 | 0.414424433 | loss | 0.588738564 | 0.174314131 | ASIC1; ASIC1; ASIC1:ASIC1 | 37 | 12 |
| cg05413199 | 7.13E−10 | 3.03E−04 | −0.414394101 | 0.414394101 | loss | 0.765082113 | 0.350688012 | ZDHHC1 | 37 | 16 |
| cg23173402 | 1.01E−09 | 4.27E−04 | −0.414365519 | 0.414365519 | loss | 0.704025819 | 0.2896603 | | 37 | 1 |
| cg21123519 | 7.12E−10 | 3.02E−04 | −0.414343062 | 0.414343062 | loss | 0.450304541 | 0.035961479 | | 37 | 14 |
| cg11070193 | 1.12E−08 | 4.76E−03 | −0.414227899 | 0.414227899 | loss | 0.570193491 | 0.155965592 | CATSPER4 | 37 | 1 |
| cg13176198 | 7.13E−10 | 3.03E−04 | −0.414000665 | 0.414000665 | loss | 0.874464247 | 0.460463582 | | 37 | 15 |
| cg18947553 | 7.13E−10 | 3.03E−04 | −0.413924582 | 0.413924582 | loss | 0.557869743 | 0.143945161 | | 37 | 1 |
| cg11237406 | 7.13E−10 | 3.03E−04 | −0.413891836 | 0.413891836 | loss | 0.789824772 | 0.375932935 | ZMYND15; ZMYND15; ZMYND15 | 37 | 17 |
| cg17674042 | 7.13E−10 | 3.03E−04 | −0.413755004 | 0.413755004 | loss | 0.717099304 | 0.3033443 | PACSIN1; PACSIN1 | 37 | 6 |
| cg17112426 | 7.13E−10 | 3.03E−04 | −0.413701892 | 0.413701892 | loss | 0.643231692 | 0.2295298 | DMKN; DMKN; DMKN; DMKN; | 37 | 19 |
| | | | | | | | | DMKN; DMKN; DMKN; | | |
| | | | | | | | | DMKN; DMKN; DMKN; DMKN; | | |
| | | | | | | | | DMKN; DMKN; DMKN | | |
| cg16503797 | 7.13E−10 | 3.03E−04 | −0.413567005 | 0.413567005 | loss | 0.743281128 | 0.329714124 | | 37 | 18 |
| cg05593775 | 7.13E−10 | 3.03E−04 | −0.413309558 | 0.413309558 | loss | 0.582218791 | 0.168909232 | PDZD7; PDZD7 | 37 | 10 |
| cg17512474 | 7.13E−10 | 3.03E−04 | −0.413080836 | 0.413080836 | loss | 0.818496194 | 0.405415359 | CDX1 | 37 | 5 |
| cg03577139 | 1.30E−09 | 5.52E−04 | −0.41303044 | 0.41303044 | loss | 0.907368975 | 0.494338535 | SOD3 | 37 | 4 |
| cg04717895 | 7.77E−10 | 3.30E−04 | −0.413016801 | 0.413016801 | loss | 0.825265483 | 0.412248682 | | 37 | 16 |
| cg21148531 | 7.13E−10 | 3.03E−04 | −0.412972872 | 0.412972872 | loss | 0.838095625 | 0.425122753 | ATG5; ATG5; ATG5; ATG5; ATG5 | 37 | 6 |
| cg08798685 | 8.80E−09 | 3.74E−03 | −0.412907935 | 0.412907935 | loss | 0.909326783 | 0.496418848 | LOC100131289 | 37 | 6 |
| cg05374238 | 1.83E−09 | 7.75E−04 | −0.412827008 | 0.412827008 | loss | 0.713456555 | 0.300629547 | C1orf168; C1orf168 | 37 | 1 |
| cg01906695 | 7.13E−10 | 3.03E−04 | −0.412585707 | 0.412585707 | loss | 0.781407319 | 0.368821612 | COL2A1; COL2A1 | 37 | 12 |
| cg17762073 | 7.13E−10 | 3.03E−04 | −0.412506989 | 0.412506989 | loss | 0.545132783 | 0.132625794 | GRM4; GRM4; GRM4; GRM4; | 37 | 5 |
| | | | | | | | | GRM4; GRM4 | | |
| cg07554053 | 6.89E−09 | 2.93E−03 | −0.412474851 | 0.412474851 | loss | 0.881330187 | 0.468855335 | | 37 | 10 |
| cg05067427 | 7.13E−10 | 3.03E−04 | −0.412356108 | 0.412356108 | loss | 0.54701827 | 0.134662162 | | 37 | 16 |
| cg10442355 | 2.16E−09 | 9.17E−04 | −0.412333013 | 0.412333013 | loss | 0.807263477 | 0.394930465 | LOC100505532; LOC100505532 | 37 | 8 |
| cg00326131 | 7.13E−10 | 3.03E−04 | −0.412149075 | 0.412149075 | loss | 0.856994392 | 0.444845318 | RTKN; RTKN; RTKN | 37 | 2 |
| cg06410849 | 7.13E−10 | 3.03E−04 | −0.411899899 | 0.411899899 | loss | 0.755861164 | 0.343961265 | RRAD; RRAD | 37 | 16 |
| cg17356252 | 7.84E−10 | 3.33E−04 | −0.411781401 | 0.411781401 | loss | 0.559022056 | 0.147240655 | AIRE | 37 | 21 |
| cg15006681 | 7.13E−10 | 3.03E−04 | −0.41163468 | 0.41163468 | loss | 0.787342962 | 0.375708282 | H1F0 | 37 | 22 |
| cg16447311 | 7.13E−10 | 3.03E−04 | −0.411615138 | 0.411615138 | loss | 0.627824626 | 0.216209488 | | 37 | 6 |
| cg25360385 | 7.13E−10 | 3.03E−04 | −0.411597568 | 0.411597568 | loss | 0.679969921 | 0.268372353 | SLC4A8; SLC4A8; GALNT6 | 37 | 12 |
| cg22623319 | 2.16E−09 | 9.17E−04 | −0.411405036 | 0.411405036 | loss | 0.825270177 | 0.413865141 | RBM20 | 37 | 10 |
| cg19512582 | 2.91E−08 | 1.23E−02 | −0.411301326 | 0.411301326 | loss | 0.724897479 | 0.313596153 | | 37 | 14 |
| cg10408284 | 7.13E−10 | 3.03E−04 | −0.411151615 | 0.411151615 | loss | 0.913123974 | 0.501972359 | WFIKKN2 | 37 | 17 |
| cg14742937 | 6.30E−08 | 2.68E−02 | −0.411006622 | 0.411006622 | loss | 0.631706864 | 0.220700242 | ADAM5 | 37 | 8 |
| cg02242734 | 7.13E−10 | 3.03E−04 | −0.410824452 | 0.410824452 | loss | 0.789181811 | 0.378357359 | SLC25A18 | 37 | 22 |
| cg04664126 | 7.13E−10 | 3.03E−04 | −0.410705346 | 0.410705346 | loss | 0.832568323 | 0.421862976 | SCAND3 | 37 | 6 |
| cg06184825 | 2.35E−09 | 9.98E−04 | −0.410671399 | 0.410671399 | loss | 0.876434434 | 0.465763035 | CPNE5 | 37 | 6 |
| cg04807108 | 7.13E−10 | 3.03E−04 | −0.410657093 | 0.410657093 | loss | 0.796545281 | 0.385888188 | | 37 | 15 |
| cg08461752 | 1.30E−09 | 5.52E−04 | −0.410468991 | 0.410468991 | loss | 0.53096063 | 0.120491639 | | 37 | 10 |
| cg05568941 | 7.13E−10 | 3.03E−04 | −0.410353311 | 0.410353311 | loss | 0.752870223 | 0.342516912 | GJD3 | 37 | 17 |
| cg09978105 | 7.13E−10 | 3.03E−04 | −0.410342951 | 0.410342951 | loss | 0.727355404 | 0.317012453 | | 37 | 14 |
| cg27446137 | 7.13E−10 | 3.03E−04 | −0.410305467 | 0.410305467 | loss | 0.590872896 | 0.180567429 | | 37 | 4 |
| cg07218647 | 7.13E−10 | 3.03E−04 | −0.410276485 | 0.410276485 | loss | 0.683730685 | 0.2734542 | | 37 | 2 |
| cg07227033 | 8.47E−10 | 3.60E−04 | −0.410165471 | 0.410165471 | loss | 0.8136009 | 0.403435429 | ARL4A; ARL4A; ARL4A; ARL4A | 37 | 7 |
| cg23305129 | 7.13E−10 | 3.03E−04 | −0.40992263 | 0.40992263 | loss | 0.492088394 | 0.082165765 | | 37 | 7 |
| cg04625615 | 7.13E−10 | 3.03E−04 | −0.409807452 | 0.409807452 | loss | 0.798716281 | 0.388908829 | ITPKA | 37 | 15 |
| cg20955328 | 7.13E−10 | 3.03E−04 | −0.409735263 | 0.409735263 | loss | 0.93125014 | 0.521514876 | KLF15 | 37 | 3 |
| cg13341668 | 7.13E−10 | 3.03E−04 | −0.409551481 | 0.409551481 | loss | 0.916513392 | 0.506961912 | HYAL2; HYAL2 | 37 | 3 |
| cg06420834 | 7.13E−10 | 3.03E−04 | −0.409397737 | 0.409397737 | loss | 0.635150455 | 0.225752718 | MYL9; MYL9 | 37 | 20 |
| cg13565129 | 7.13E−10 | 3.03E−04 | −0.409381638 | 0.409381638 | loss | 0.590422494 | 0.181040856 | | 37 | 6 |
| cg19782271 | 7.13E−10 | 3.03E−04 | −0.409350234 | 0.409350234 | loss | 0.718546787 | 0.309196553 | STK10 | 37 | 5 |
| cg02257791 | 4.21E−09 | 1.79E−03 | −0.409297413 | 0.409297413 | loss | 0.681314685 | 0.272017272 | | 37 | 14 |
| cg27537199 | 7.13E−10 | 3.03E−04 | −0.409111992 | 0.409111992 | loss | 0.586427192 | 0.1773152 | | 37 | 1 |
| cg00220225 | 7.13E−10 | 3.03E−04 | −0.409060416 | 0.409060416 | loss | 0.71148684 | 0.302426424 | SEMA3F | 37 | 3 |
| cg18137414 | 7.84E−10 | 3.33E−04 | −0.408929932 | 0.408929932 | loss | 0.907737079 | 0.498807147 | | 37 | 22 |
| cg14190522 | 7.13E−10 | 3.03E−04 | −0.408818863 | 0.408818863 | loss | 0.664406798 | 0.255587935 | DAB2IP | 37 | 9 |
| cg06117341 | 7.13E−10 | 3.03E−04 | −0.408812834 | 0.408812834 | loss | 0.520954426 | 0.112141592 | CLDN9 | 37 | 16 |
| cg02547167 | 7.77E−10 | 3.30E−04 | −0.408796452 | 0.408796452 | loss | 0.753583581 | 0.344787129 | | 37 | X |
| cg03609493 | 7.13E−10 | 3.03E−04 | −0.408654457 | 0.408654457 | loss | 0.466323123 | 0.057668665 | MIR572 | 37 | 4 |
| cg17736526 | 7.13E−10 | 3.03E−04 | −0.408346702 | 0.408346702 | loss | 0.617940308 | 0.209593606 | | 37 | 7 |
| cg19873297 | 7.13E−10 | 3.03E−04 | −0.40798267 | 0.40798267 | loss | 0.924282111 | 0.516299441 | STK10 | 37 | 5 |
| cg00668150 | 7.13E−10 | 3.03E−04 | −0.407936531 | 0.407936531 | loss | 0.683196566 | 0.275260035 | CLUH | 37 | 17 |
| cg14478475 | 7.13E−10 | 3.03E−04 | −0.407934321 | 0.407934321 | loss | 0.887822262 | 0.479887941 | SLC25A51; SLC25A51; SLC25A51 | 37 | 9 |
| cg25415695 | 7.13E−10 | 3.03E−04 | −0.407881824 | 0.407881824 | loss | 0.794696877 | 0.386815053 | | 37 | 5 |
| cg14920044 | 7.13E−10 | 3.03E−04 | −0.407550848 | 0.407550848 | loss | 0.79054613 | 0.382995282 | SLC16A12 | 37 | 10 |
| cg22275125 | 7.13E−10 | 3.03E−04 | −0.407488736 | 0.407488736 | loss | 0.899437936 | 0.4919492 | SLC22A8; SLC22A8; SLC22A8; | 37 | 11 |
| | | | | | | | | SLC22A8 | | |
| cg21793452 | 7.77E−10 | 3.30E−04 | −0.407371089 | 0.407371089 | loss | 0.825719536 | 0.418348447 | | 37 | 3 |
| cg15123755 | 7.13E−10 | 3.03E−04 | −0.407294144 | 0.407294144 | loss | 0.786320685 | 0.379026541 | | 37 | 1 |
| cg00610692 | 7.13E−10 | 3.03E−04 | −0.406983075 | 0.406983075 | loss | 0.656368775 | 0.2493857 | RTKN; RTKN; RTKN | 37 | 2 |
| cg17052170 | 7.13E−10 | 3.03E−04 | −0.406979262 | 0.406979262 | loss | 0.790862326 | 0.383883065 | LY6E; LY6E; LOC100133669 | 37 | 8 |
| cg17454857 | 7.13E−10 | 3.03E−04 | −0.406901709 | 0.406901709 | loss | 0.813011774 | 0.406110065 | | 37 | 20 |
| cg24176731 | 7.13E−10 | 3.03E−04 | −0.406860848 | 0.406860848 | loss | 0.88418273 | 0.477321882 | PLEKHB1; PLEKHB1; PLEKHB1; | 37 | 11 |
| | | | | | | | | PLEKHB1 | | |
| cg16667508 | 7.13E−10 | 3.03E−04 | −0.406444502 | 0.406444502 | loss | 0.759774849 | 0.353330347 | RBPJL; RBPJL; MATN4; RBPJL; | 37 | 20 |
| | | | | | | | | MATN4; MATN4; MATN4; | | |
| | | | | | | | | MATN4 | | |
| cg00079785 | 7.13E−10 | 3.03E−04 | −0.406379365 | 0.406379365 | loss | 0.935071889 | 0.528692524 | SPIDR; SPIDR; SPIDR; SPIDR | 37 | 8 |
| cg15701111 | 7.84E−10 | 3.33E−04 | −0.406197674 | 0.406197674 | loss | 0.805412815 | 0.399215141 | KRT81; KRT81 | 37 | 12 |
| cg11322879 | 7.13E−10 | 3.03E−04 | −0.406153022 | 0.406153022 | loss | 0.688684492 | 0.282531471 | | 37 | 7 |
| cg02896835 | 7.13E−10 | 3.03E−04 | −0.406100667 | 0.406100667 | loss | 0.910141332 | 0.504040665 | | 37 | 1 |
| cg16624888 | 7.13E−10 | 3.03E−04 | −0.406087208 | 0.406087208 | loss | 0.547314704 | 0.141227496 | | 37 | 12 |
| cg18971434 | 7.13E−10 | 3.03E−04 | −0.405977842 | 0.405977842 | loss | 0.841466425 | 0.435488582 | LOC401242 | 37 | 6 |
| cg25432738 | 7.13E−10 | 3.03E−04 | −0.405824157 | 0.405824157 | loss | 0.801314472 | 0.395490315 | CRYL1 | 37 | 13 |
| cg15175162 | 7.13E−10 | 3.03E−04 | −0.405791703 | 0.405791703 | loss | 0.604884732 | 0.199093029 | FBXL5; FBXL5; FBXL5; FBXL5 | 37 | 4 |
| cg05366561 | 7.13E−10 | 3.03E−04 | −0.405768298 | 0.405768298 | loss | 0.491718094 | 0.085949796 | | 37 | 20 |
| cg12043145 | 2.03E−09 | 8.64E−04 | −0.405561784 | 0.405561784 | loss | 0.901956908 | 0.496395124 | | 37 | 14 |
| cg13207630 | 8.47E−10 | 3.60E−04 | −0.405552422 | 0.405552422 | loss | 0.5106785 | 0.105126078 | | 37 | 7 |
| cg18604823 | 7.13E−10 | 3.03E−04 | −0.405429628 | 0.405429628 | loss | 0.77693174 | 0.371502112 | GALP; GALP | 37 | 19 |
| cg14644378 | 7.13E−10 | 3.03E−04 | −0.405268264 | 0.405268264 | loss | 0.594201683 | 0.188933419 | DNAJB5; DNAJB5 | 37 | 9 |
| cg24496745 | 1.03E−08 | 4.39E−03 | −0.405200062 | 0.405200062 | loss | 0.786081862 | 0.3808818 | C1orf168 | 37 | 1 |
| cg00054210 | 7.13E−10 | 3.03E−04 | −0.405007555 | 0.405007555 | loss | 0.449233164 | 0.044225609 | CYP7B1 | 37 | 8 |
| cg25001930 | 7.13E−10 | 3.03E−04 | −0.404814171 | 0.404814171 | loss | 0.566978049 | 0.162163878 | NBPF8; NBPF8; NBPF8 | 37 | 1 |
| cg02920396 | 7.13E−10 | 3.03E−04 | −0.404712488 | 0.404712488 | loss | 0.748232 | 0.343519512 | MRGPRF; MRGPRF | 37 | 11 |
| cg04896115 | 8.47E−10 | 3.60E−04 | −0.404670391 | 0.404670391 | loss | 0.751294185 | 0.346623794 | | 37 | 7 |
| cg17886420 | 1.54E−09 | 6.54E−04 | −0.404586301 | 0.404586301 | loss | 0.627067419 | 0.222481118 | | 37 | 10 |
| cg11757040 | 7.13E−10 | 3.03E−04 | −0.404443569 | 0.404443569 | loss | 0.875537951 | 0.471094382 | DPF3; DPF3; DPF3; DPF3 | 37 | 14 |
| cg00689340 | 7.13E−10 | 3.03E−04 | −0.404350927 | 0.404350927 | loss | 0.579048109 | 0.174697182 | RTKN; RTKN; RTKN | 37 | 2 |
| cg17774102 | 7.13E−10 | 3.03E−04 | −0.404311504 | 0.404311504 | loss | 0.921503709 | 0.517192206 | LOC401242 | 37 | 6 |
| cg07298985 | 1.01E−09 | 4.27E−04 | −0.404286902 | 0.404286902 | loss | 0.890352955 | 0.486066053 | PIWIL2; PIWIL2 | 37 | 8 |
| cg25838465 | 8.47E−10 | 3.60E−04 | −0.40425138 | 0.40425138 | loss | 0.820099268 | 0.415847888 | | 37 | 1 |
| cg06599908 | 7.13E−10 | 3.03E−04 | −0.404232175 | 0.404232175 | loss | 0.519785872 | 0.115553697 | TTYH1; TTYH1; TTYH1 | 37 | 19 |
| cg09113483 | 1.10E−09 | 4.65E−04 | −0.404139049 | 0.404139049 | loss | 0.680534691 | 0.276395641 | | 37 | 1 |
| cg16034541 | 4.29E−08 | 1.82E−02 | −0.404064572 | 0.404064572 | loss | 0.576750858 | 0.172686286 | MUC4; MUC4; MUC4 | 37 | 3 |
| cg26644853 | 7.13E−10 | 3.03E−04 | −0.404001239 | 0.404001239 | loss | 0.665886615 | 0.261885376 | CX3CL1 | 37 | 16 |
| cg22350048 | 7.13E−10 | 3.03E−04 | −0.40385164 | 0.40385164 | loss | 0.60954687 | 0.205695229 | | 37 | 1 |
| cg03727500 | 7.13E−10 | 3.03E−04 | −0.403827582 | 0.403827582 | loss | 0.533839808 | 0.130012226 | | 37 | 2 |
| cg05463589 | 7.77E−10 | 3.30E−04 | −0.40345929 | 0.40345929 | loss | 0.638931606 | 0.235472315 | IL17C | 37 | 16 |
| cg09214243 | 8.47E−10 | 3.60E−04 | −0.403455257 | 0.403455257 | loss | 0.50689877 | 0.103443513 | | 37 | 15 |
| cg17827650 | 7.13E−10 | 3.03E−04 | −0.403421942 | 0.403421942 | loss | 0.644192019 | 0.240770076 | NOM1 | 37 | 7 |
| cg02987388 | 7.13E−10 | 3.03E−04 | −0.403419369 | 0.403419369 | loss | 0.901483998 | 0.498064629 | CCDC17; CCDC17 | 37 | 1 |
| cg21160372 | 7.13E−10 | 3.03E−04 | −0.403301271 | 0.403301271 | loss | 0.734657142 | 0.331355871 | NCAM2 | 37 | 21 |
| cg05254747 | 7.13E−10 | 3.03E−04 | −0.403176137 | 0.403176137 | loss | 0.694615008 | 0.291438871 | SLC39A14; SLC39A14; SLC39A14; | 37 | 8 |
| | | | | | | | | SLC39A14 | | |
| cg15116481 | 7.13E−10 | 3.03E−04 | −0.403148266 | 0.403148266 | loss | 0.841477619 | 0.438329353 | KLK15; KLK15; KLK15; KLK15 | 37 | 19 |
| cg00913360 | 2.16E−09 | 9.17E−04 | −0.402657971 | 0.402657971 | loss | 0.788295547 | 0.385637576 | | 37 | 7 |
| cg13981310 | 7.13E−10 | 3.03E−04 | −0.40264187 | 0.40264187 | loss | 0.772631758 | 0.369989888 | ENTPD2; ENTPD2 | 37 | 9 |
| cg04566512 | 7.13E−10 | 3.03E−04 | −0.402620369 | 0.402620369 | loss | 0.825267323 | 0.422646954 | | 37 | 22 |
| cg25653641 | 7.13E−10 | 3.03E−04 | −0.402585568 | 0.402585568 | loss | 0.691797774 | 0.289212206 | | 37 | 6 |
| cg18675097 | 7.13E−10 | 3.03E−04 | −0.402465573 | 0.402465573 | loss | 0.549688142 | 0.147222569 | NKAPL | 37 | 6 |
| cg00772991 | 7.13E−10 | 3.03E−04 | −0.40244414 | 0.40244414 | loss | 0.671555475 | 0.269111335 | | 37 | 2 |
| cg13655803 | 7.13E−10 | 3.03E−04 | −0.402303397 | 0.402303397 | loss | 0.625353274 | 0.223049876 | WFIKKN2 | 37 | 17 |
| cg17295878 | 7.13E−10 | 3.03E−04 | −0.402212078 | 0.402212078 | loss | 0.738870249 | 0.336658171 | TBC1D16; TBC1D16; TBC1D16; | 37 | 17 |
| | | | | | | | | TBC1D16; TBC1D16; TBC1D16; | | |
| | | | | | | | | TBC1D16 | | |
| cg23109344 | 7.13E−10 | 3.03E−04 | −0.402144807 | 0.402144807 | loss | 0.787032183 | 0.384887376 | TDRP | 37 | 8 |
| cg18919720 | 7.84E−10 | 3.33E−04 | −0.401656347 | 0.401656347 | loss | 0.741592888 | 0.339936541 | FSCN2; FSCN2 | 37 | 17 |
| cg02002217 | 7.13E−10 | 3.03E−04 | −0.401642085 | 0.401642085 | loss | 0.726420291 | 0.324778206 | ANKRD20A19P | 37 | 13 |
| cg05485462 | 7.13E−10 | 3.03E−04 | −0.401553346 | 0.401553346 | loss | 0.749882834 | 0.348329488 | CLDN9 | 37 | 16 |
| cg09240693 | 7.13E−10 | 3.03E−04 | −0.401486091 | 0.401486091 | loss | 0.660677021 | 0.259190929 | | 37 | 2 |
| cg03558837 | 7.13E−10 | 3.03E−04 | −0.401452099 | 0.401452099 | loss | 0.651931175 | 0.250479076 | ESPNL | 37 | 2 |
| cg13583664 | 7.13E−10 | 3.03E−04 | −0.401331375 | 0.401331375 | loss | 0.885723528 | 0.484392153 | | 37 | 9 |
| cg25571880 | 7.13E−10 | 3.03E−04 | −0.401223748 | 0.401223748 | loss | 0.673970866 | 0.272747118 | PRSS56 | 37 | 2 |
| cg06106599 | 8.65E−10 | 3.67E−04 | −0.401141044 | 0.401141044 | loss | 0.741877702 | 0.340736658 | FADD | 37 | 11 |
| cg09083345 | 7.13E−10 | 3.03E−04 | −0.400991093 | 0.400991093 | loss | 0.72297994 | 0.321988847 | | 37 | 6 |
| cg02754470 | 7.13E−10 | 3.03E−04 | −0.40098105 | 0.40098105 | loss | 0.766370162 | 0.365389112 | | 37 | 15 |
| cg24516106 | 4.75E−09 | 2.02E−03 | −0.400881633 | 0.400881633 | loss | 0.698180908 | 0.297299275 | | 37 | 2 |
| cg25556225 | 1.03E−08 | 4.39E−03 | −0.40078962 | 0.40078962 | loss | 0.830150532 | 0.429360912 | LOC283710 | 37 | 15 |
| cg09336899 | 7.13E−10 | 3.03E−04 | −0.400496594 | 0.400496594 | loss | 0.852927511 | 0.452430918 | NGEF; NGEF | 37 | 2 |
| cg11737757 | 7.13E−10 | 3.03E−04 | −0.400399889 | 0.400399889 | loss | 0.587832125 | 0.187432235 | | 37 | 1 |
| cg15868235 | 7.84E−10 | 3.33E−04 | −0.400186592 | 0.400186592 | loss | 0.75940191 | 0.359215318 | | 37 | 3 |
| cg14972141 | 7.13E−10 | 3.03E−04 | −0.400167327 | 0.400167327 | loss | 0.823663321 | 0.423495994 | | 37 | 13 |
| cg05415308 | 7.13E−10 | 3.03E−04 | −0.400146765 | 0.400146765 | loss | 0.744783547 | 0.344636782 | CT62 | 37 | 15 |
| cg12813394 | 7.13E−10 | 3.03E−04 | −0.400039143 | 0.400039143 | loss | 0.433263394 | 0.033224251 | | 37 | 14 |
| cg17136073 | 7.13E−10 | 3.03E−04 | −0.399866511 | 0.399866511 | loss | 0.616017358 | 0.216150847 | | 37 | 13 |
| cg09820729 | 7.13E−10 | 3.03E−04 | −0.399609432 | 0.399609432 | loss | 0.712025074 | 0.312415641 | MFN2; MFN2 | 37 | 1 |
| cg17681516 | 7.13E−10 | 3.03E−04 | −0.399423891 | 0.399423891 | loss | 0.910999938 | 0.511576047 | | 37 | 2 |
| cg00176638 | 8.47E−10 | 3.60E−04 | −0.39923979 | 0.39923979 | loss | 0.767451143 | 0.368211353 | KDM5B | 37 | 1 |
| cg21186576 | 7.13E−10 | 3.03E−04 | −0.399192931 | 0.399192931 | loss | 0.82518026 | 0.425987329 | | 37 | 17 |
| cg06813382 | 7.13E−10 | 3.03E−04 | −0.399087012 | 0.399087012 | loss | 0.601754189 | 0.202667176 | LINC00857 | 37 | 10 |
| cg13655082 | 7.13E−10 | 3.03E−04 | −0.398724092 | 0.398724092 | loss | 0.516756726 | 0.118032634 | | 37 | 9 |
| cg10504392 | 7.13E−10 | 3.03E−04 | −0.398608545 | 0.398608545 | loss | 0.519117315 | 0.120508771 | | 37 | 12 |
| cg22717478 | 8.56E−10 | 3.63E−04 | −0.398562469 | 0.398562469 | loss | 0.876942404 | 0.478379935 | OVCH1-AS1; OVCH1- | 37 | 12 |
| | | | | | | | | AS1; OVCH1-AS1 | | |
| cg09133892 | 7.77E−10 | 3.30E−04 | −0.398537483 | 0.398537483 | loss | 0.833143325 | 0.434605841 | LOC100505532 | 37 | 8 |
| cg06911613 | 1.01E−09 | 4.27E−04 | −0.398518831 | 0.398518831 | loss | 0.664869666 | 0.266350835 | | 37 | 16 |
| cg02623028 | 7.13E−10 | 3.03E−04 | −0.398278459 | 0.398278459 | loss | 0.829650547 | 0.431372088 | | 37 | 1 |
| cg25942596 | 6.89E−09 | 2.93E−03 | −0.398250449 | 0.398250449 | loss | 0.676359196 | 0.278108747 | | 37 | 15 |
| cg00224807 | 8.47E−10 | 3.60E−04 | −0.398196937 | 0.398196937 | loss | 0.892560455 | 0.494363518 | | 37 | 2 |
| cg08580014 | 1.89E−09 | 8.02E−04 | −0.398105183 | 0.398105183 | loss | 0.672282364 | 0.274177181 | PNLIPRP1 | 37 | 10 |
| cg07412254 | 7.13E−10 | 3.03E−04 | −0.39799628 | 0.39799628 | loss | 0.749020668 | 0.351024388 | LINC01101 | 37 | 2 |
| cg01337207 | 7.77E−10 | 3.30E−04 | −0.397977533 | 0.397977533 | loss | 0.586902464 | 0.188924931 | TNXB | 37 | 6 |
| cg13640071 | 9.23E−10 | 3.92E−04 | −0.397681317 | 0.397681317 | loss | 0.710372785 | 0.312691468 | TCEAL7 | 37 | X |
| cg27243166 | 7.13E−10 | 3.03E−04 | −0.397642371 | 0.397642371 | loss | 0.794677189 | 0.397034818 | NRXN2; NRXN2 | 37 | 11 |
| cg14563637 | 7.13E−10 | 3.03E−04 | −0.397637695 | 0.397637695 | loss | 0.748662683 | 0.351024988 | | 37 | 9 |
| cg20219329 | 4.57E−09 | 1.94E−03 | −0.39763027 | 0.39763027 | loss | 0.655223364 | 0.257593094 | MYOM2 | 37 | 8 |
| cg12414681 | 7.13E−10 | 3.03E−04 | −0.397605288 | 0.397605288 | loss | 0.647044734 | 0.249439446 | | 37 | 15 |
| cg04206219 | 7.13E−10 | 3.03E−04 | −0.397470739 | 0.397470739 | loss | 0.739935168 | 0.342464429 | | 37 | 6 |
| cg24437523 | 7.13E−10 | 3.03E−04 | −0.397392843 | 0.397392843 | loss | 0.690826255 | 0.293433412 | ATP1A2 | 37 | 1 |
| cg09203312 | 8.47E−10 | 3.60E−04 | −0.397226885 | 0.397226885 | loss | 0.668253421 | 0.271026535 | GJB6; GJB6; GJB6; GJB6; GJB6 | 37 | 13 |
| cg12428514 | 3.57E−09 | 1.52E−03 | −0.397181463 | 0.397181463 | loss | 0.925528887 | 0.528347424 | UCN3 | 37 | 10 |
| cg13073141 | 7.13E−10 | 3.03E−04 | −0.396611693 | 0.396611693 | loss | 0.685326281 | 0.288714588 | CERS2; CERS2 | 37 | 1 |
| cg11343894 | 7.48E−09 | 3.18E−03 | −0.396429528 | 0.396429528 | loss | 0.549655619 | 0.153226091 | S100A13; S100A13; S100A13; | 37 | 1 |
| | | | | | | | | S100A13; S100A13; S100A13; | | |
| | | | | | | | | S100A13; S100A1 | | |
| cg18081818 | 7.13E−10 | 3.03E−04 | −0.396382244 | 0.396382244 | loss | 0.769948938 | 0.373566694 | | 37 | 7 |
| cg09644356 | 7.13E−10 | 3.03E−04 | −0.396316097 | 0.396316097 | loss | 0.837932621 | 0.441616524 | | 37 | 2 |
| cg00568384 | 1.01E−09 | 4.27E−04 | −0.396248034 | 0.396248034 | loss | 0.661890328 | 0.265642294 | | 37 | 2 |
| cg20495964 | 3.57E−09 | 1.52E−03 | −0.396239976 | 0.396239976 | loss | 0.805546311 | 0.409306335 | CPNE5 | 37 | 6 |
| cg27508620 | 7.13E−10 | 3.03E−04 | −0.396203275 | 0.396203275 | loss | 0.758023487 | 0.361820212 | | 37 | 7 |
| cg21572914 | 7.13E−10 | 3.03E−04 | −0.395993653 | 0.395993653 | loss | 0.9479293 | 0.551935647 | LOC401242 | 37 | 6 |
| cg26575633 | 7.13E−10 | 3.03E−04 | −0.395811856 | 0.395811856 | loss | 0.577134879 | 0.181323024 | NTRK1; NTRK1; NTRK1 | 37 | 1 |
| cg27211198 | 7.13E−10 | 3.03E−04 | −0.395807102 | 0.395807102 | loss | 0.512150255 | 0.116343152 | | 37 | 2 |
| cg03619568 | 7.13E−10 | 3.03E−04 | −0.395331431 | 0.395331431 | loss | 0.828274802 | 0.432943371 | | 37 | 5 |
| cg15478483 | 7.13E−10 | 3.03E−04 | −0.395082353 | 0.395082353 | loss | 0.7286728 | 0.333590447 | | 37 | 6 |
| cg13142134 | 7.13E−10 | 3.03E−04 | −0.395035031 | 0.395035031 | loss | 0.512191536 | 0.117156505 | ATP6V0CP3 | 37 | 6 |
| cg03701930 | 1.68E−09 | 7.12E−04 | 0.3949729 | 0.3949729 | gain | 0.244566394 | 0.639539294 | | 37 | 10 |
| cg22022881 | 1.54E−09 | 6.54E−04 | −0.394961901 | 0.394961901 | loss | 0.632726194 | 0.237764294 | | 37 | 2 |
| cg07499899 | 7.13E−10 | 3.03E−04 | −0.394938052 | 0.394938052 | loss | 0.963354464 | 0.568416412 | RAP1GAP; RAP1GAP; RAP1GAP | 37 | 1 |
| cg01234610 | 7.13E−10 | 3.03E−04 | −0.394729601 | 0.394729601 | loss | 0.857845266 | 0.463115665 | WFIKKN2 | 37 | 17 |
| cg03335125 | 7.13E−10 | 3.03E−04 | −0.394700378 | 0.394700378 | loss | 0.924679713 | 0.529979335 | CCDC17; CCDC17 | 37 | 1 |
| cg18976044 | 7.77E−10 | 3.30E−04 | −0.394648154 | 0.394648154 | loss | 0.715932966 | 0.321284812 | ANKRD20A19P | 37 | 13 |
| cg00718036 | 7.13E−10 | 3.03E−04 | −0.394594822 | 0.394594822 | loss | 0.888177045 | 0.493582224 | SGMS2 | 37 | 4 |
| cg04972348 | 7.13E−10 | 3.03E−04 | −0.394575872 | 0.394575872 | loss | 0.747829613 | 0.353253741 | | 37 | 1 |
| cg12487162 | 5.39E−09 | 2.29E−03 | −0.394543457 | 0.394543457 | loss | 0.779135545 | 0.384592088 | TEAD1 | 37 | 11 |
| cg21181453 | 8.47E−10 | 3.60E−04 | −0.394377621 | 0.394377621 | loss | 0.756702815 | 0.362325194 | DPM2; DPM2 | 37 | 9 |
| cg18174881 | 7.13E−10 | 3.03E−04 | −0.394202116 | 0.394202116 | loss | 0.796916698 | 0.402714582 | MIS18BP1 | 37 | 14 |
| cg14736365 | 1.42E−09 | 6.01E−04 | −0.394093384 | 0.394093384 | loss | 0.640644508 | 0.246551124 | | 37 | 5 |
| cg24690588 | 1.68E−09 | 7.12E−04 | −0.394070834 | 0.394070834 | loss | 0.759738175 | 0.365667341 | C1orf168; C1orf168 | 37 | 1 |
| cg05096492 | 8.47E−10 | 3.60E−04 | −0.394068209 | 0.394068209 | loss | 0.704714179 | 0.310645971 | LOC401242 | 37 | 6 |
| cg13588403 | 7.13E−10 | 3.03E−04 | −0.393735783 | 0.393735783 | loss | 0.884940777 | 0.491204994 | DPF3; DPF3; DPF3; DPF3 | 37 | 14 |
| cg09187505 | 7.77E−10 | 3.30E−04 | −0.393618291 | 0.393618291 | loss | 0.531076983 | 0.137458692 | | 37 | 6 |
| cg03583746 | 7.13E−10 | 3.03E−04 | −0.393544485 | 0.393544485 | loss | 0.830871038 | 0.437326553 | | 37 | 6 |
| cg24911721 | 5.85E−09 | 2.49E−03 | −0.39346161 | 0.39346161 | loss | 0.816858 | 0.42339639 | MIRLET7BHG; MIRLET7A3; | 37 | 22 |
| | | | | | | | | MIRLET7B; MIR4763 | | |
| cg21544713 | 1.10E−09 | 4.65E−04 | −0.39343862 | 0.39343862 | loss | 0.859181449 | 0.465742829 | LOC100505532; LOC100505532 | 37 | 8 |
| cg09355528 | 7.77E−10 | 3.30E−04 | −0.393434856 | 0.393434856 | loss | 0.815718068 | 0.422283212 | COL13A1; COL13A1; COL13A1; | 37 | 10 |
| | | | | | | | | COL13A1; COL13A1; COL13A1 | | |
| cg22619549 | 2.16E−09 | 9.17E−04 | −0.393431247 | 0.393431247 | loss | 0.907876353 | 0.514445106 | PKD2L1; PKD2L1; PKD2L1; | 37 | 10 |
| | | | | | | | | PKD2L1 | | |
| cg22816091 | 1.01E−09 | 4.27E−04 | −0.393141028 | 0.393141028 | loss | 0.691208292 | 0.298067265 | | 37 | 1 |
| cg22877452 | 9.23E−10 | 3.92E−04 | −0.393117988 | 0.393117988 | loss | 0.776638864 | 0.383520876 | | 37 | 1 |
| cg25318971 | 7.13E−10 | 3.03E−04 | −0.39275964 | 0.39275964 | loss | 0.735260534 | 0.342500894 | BMP7 | 37 | 20 |
| cg25937216 | 9.23E−10 | 3.92E−04 | −0.392726329 | 0.392726329 | loss | 0.822442723 | 0.429716394 | LOC286177 | 37 | 8 |
| cg00937681 | 7.13E−10 | 3.03E−04 | −0.392534593 | 0.392534593 | loss | 0.618807381 | 0.226272788 | | 37 | 4 |
| cg27539527 | 7.13E−10 | 3.03E−04 | −0.392524602 | 0.392524602 | loss | 0.506519525 | 0.113994923 | | 37 | 7 |
| cg06590074 | 7.13E−10 | 3.03E−04 | −0.392080697 | 0.392080697 | loss | 0.873765885 | 0.481685188 | | 37 | 20 |
| cg05866021 | 1.99E−09 | 8.43E−04 | −0.392058969 | 0.392058969 | loss | 0.87266184 | 0.480602871 | C1orf168; C1orf168 | 37 | 1 |
| cg13883721 | 7.13E−10 | 3.03E−04 | −0.391926115 | 0.391926115 | loss | 0.824160009 | 0.432233894 | | 37 | 9 |
| cg02994923 | 7.13E−10 | 3.03E−04 | −0.391853317 | 0.391853317 | loss | 0.796443706 | 0.404590388 | | 37 | 2 |
| cg20310608 | 1.73E−09 | 7.34E−04 | −0.391828483 | 0.391828483 | loss | 0.789681926 | 0.397853444 | LOC284798; LOC284798; LOC284798; | 37 | 20 |
| | | | | | | | | LOC284798 | | |
| cg27399125 | 2.91E−08 | 1.23E−02 | −0.391567018 | 0.391567018 | loss | 0.8665794 | 0.475012382 | | 37 | 6 |
| cg26587870 | 5.85E−09 | 2.49E−03 | −0.391448836 | 0.391448836 | loss | 0.935265145 | 0.543816309 | LOC100131289 | 37 | 6 |
| cg19537511 | 7.77E−10 | 3.30E−04 | −0.391404538 | 0.391404538 | loss | 0.702359532 | 0.310954994 | ARHGEF15; ARHGEF15 | 37 | 17 |
| cg11934832 | 1.54E−08 | 6.56E−03 | −0.391387038 | 0.391387038 | loss | 0.812276438 | 0.4208894 | | 37 | 18 |
| cg24657518 | 7.84E−10 | 3.33E−04 | −0.391175105 | 0.391175105 | loss | 0.857733346 | 0.466558241 | | 37 | 6 |
| cg04236639 | 7.13E−10 | 3.03E−04 | −0.391131713 | 0.391131713 | loss | 0.87483916 | 0.483707447 | | 37 | 21 |
| cg15644756 | 7.13E−10 | 3.03E−04 | −0.390820506 | 0.390820506 | loss | 0.966063706 | 0.5752432 | LOC100130075 | 37 | 12 |
| cg00934322 | 7.13E−10 | 3.03E−04 | −0.390819298 | 0.390819298 | loss | 0.836436762 | 0.445617465 | DDR1; DDR1; DDR1 | 37 | 6 |
| cg06633543 | 7.13E−10 | 3.03E−04 | −0.390799283 | 0.390799283 | loss | 0.783634336 | 0.392835053 | CYP46A1 | 37 | 14 |
| cg05924882 | 6.89E−09 | 2.93E−03 | −0.390702341 | 0.390702341 | loss | 0.726370921 | 0.335668579 | | 37 | 15 |
| cg17906007 | 7.13E−10 | 3.03E−04 | −0.390648588 | 0.390648588 | loss | 0.647356206 | 0.256707618 | RASD2 | 37 | 22 |
| cg14686645 | 7.13E−10 | 3.03E−04 | −0.390627894 | 0.390627894 | loss | 0.506222026 | 0.115594132 | ITGA2B | 37 | 17 |
| cg11883009 | 7.13E−10 | 3.03E−04 | −0.390594671 | 0.390594671 | loss | 0.893495347 | 0.502900676 | NFIX; NFIX; NFIX | 37 | 19 |
| cg05991492 | 7.84E−10 | 3.33E−04 | −0.390584279 | 0.390584279 | loss | 0.454609075 | 0.064024796 | | 37 | 16 |
| cg15720535 | 7.77E−10 | 3.30E−04 | −0.390201811 | 0.390201811 | loss | 0.471366155 | 0.081164344 | AGPAT2; AGPAT2 | 37 | 9 |
| cg26314966 | 7.13E−10 | 3.03E−04 | −0.390066814 | 0.390066814 | loss | 0.804513855 | 0.414447041 | | 37 | 17 |
| cg20593826 | 7.13E−10 | 3.03E−04 | −0.389899709 | 0.389899709 | loss | 0.807506179 | 0.417606471 | LOC100131060; LOC100131060 | 37 | 1 |
| cg24998527 | 5.39E−09 | 2.29E−03 | −0.389685984 | 0.389685984 | loss | 0.617377298 | 0.227691314 | | 37 | 1 |
| cg23638640 | 1.10E−09 | 4.65E−04 | −0.389630849 | 0.389630849 | loss | 0.887660513 | 0.498029665 | PIWIL3; PIWIL3; TOP1P2; PIWIL3; | 37 | 22 |
| | | | | | | | | PIWIL3 | | |
| cg06203364 | 7.13E−10 | 3.03E−04 | −0.389498091 | 0.389498091 | loss | 0.553291438 | 0.163793347 | C1orf86; C1orf86 | 37 | 1 |
| cg07371589 | 7.13E−10 | 3.03E−04 | −0.38942505 | 0.38942505 | loss | 0.816352385 | 0.426927335 | SEMA6C; SEMA6C; SEMA6C | 37 | 1 |
| cg24696183 | 7.13E−10 | 3.03E−04 | −0.389375162 | 0.389375162 | loss | 0.766508962 | 0.3771338 | KCNQ1DN | 37 | 11 |
| cg02941741 | 7.13E−10 | 3.03E−04 | −0.389374041 | 0.389374041 | loss | 0.769341117 | 0.379967076 | NGEF; NGEF | 37 | 2 |
| cg05215994 | 7.13E−10 | 3.03E−04 | −0.389370059 | 0.389370059 | loss | 0.773051436 | 0.383681376 | | 37 | 13 |
| cg23989336 | 7.13E−10 | 3.03E−04 | −0.389334547 | 0.389334547 | loss | 0.745892847 | 0.3565583 | LINC00482 | 37 | 17 |
| cg26624732 | 2.91E−08 | 1.23E−02 | −0.389192807 | 0.389192807 | loss | 0.804315225 | 0.415122418 | | 37 | 5 |
| cg10082647 | 7.13E−10 | 3.03E−04 | −0.389165726 | 0.389165726 | loss | 0.802219826 | 0.4130541 | C12orf23 | 37 | 12 |
| cg27520536 | 7.13E−10 | 3.03E−04 | −0.38910318 | 0.38910318 | loss | 0.849619298 | 0.460516118 | | 37 | 16 |
| cg26787381 | 7.13E−10 | 3.03E−04 | −0.388872467 | 0.388872467 | loss | 0.697841685 | 0.308969218 | LOC284798; LOC284798; LOC284798; | 37 | 20 |
| | | | | | | | | LOC284798 | | |
| cg06188367 | 7.13E−10 | 3.03E−04 | −0.388801683 | 0.388801683 | loss | 0.586675906 | 0.197874223 | TMEM72-AS1 | 37 | 10 |
| cg19659741 | 6.30E−08 | 2.68E−02 | −0.388754317 | 0.388754317 | loss | 0.681830211 | 0.293075894 | ADAM5 | 37 | 8 |
| cg25830307 | 7.13E−10 | 3.03E−04 | −0.388729163 | 0.388729163 | loss | 0.916093134 | 0.527363971 | C10orf11 | 37 | 10 |
| cg18524788 | 1.73E−09 | 7.34E−04 | −0.388448859 | 0.388448859 | loss | 0.924311691 | 0.535862831 | | 37 | 7 |
| cg13870494 | 9.23E−10 | 3.92E−04 | −0.388365045 | 0.388365045 | loss | 0.819151851 | 0.430786806 | MAMDC2 | 37 | 9 |
| cg21117965 | 1.01E−09 | 4.27E−04 | −0.388293383 | 0.388293383 | loss | 0.634915153 | 0.24662177 | SPEG; SPEG | 37 | 2 |
| cg05897163 | 7.13E−10 | 3.03E−04 | −0.388250228 | 0.388250228 | loss | 0.855860498 | 0.467610271 | PPP1R1B | 37 | 17 |
| cg24795825 | 7.13E−10 | 3.03E−04 | −0.388218638 | 0.388218638 | loss | 0.506964643 | 0.118746006 | MORF4L1; MORF4L1; MORF4L1; | 37 | 15 |
| | | | | | | | | MORF4L1; MORF4L1 | | |
| cg02049180 | 7.13E−10 | 3.03E−04 | −0.388100624 | 0.388100624 | loss | 0.860645147 | 0.472544524 | NTRK1; INSRR | 37 | 1 |
| cg02873315 | 9.23E−10 | 3.92E−04 | −0.388067558 | 0.388067558 | loss | 0.653902817 | 0.265835259 | | 37 | 10 |
| cg22316093 | 7.13E−10 | 3.03E−04 | −0.388056949 | 0.388056949 | loss | 0.797636195 | 0.409579247 | | 37 | 21 |
| cg04308185 | 7.13E−10 | 3.03E−04 | −0.388014688 | 0.388014688 | loss | 0.671114394 | 0.283099706 | ORMDL3 | 37 | 17 |
| cg14453762 | 7.84E−10 | 3.33E−04 | −0.38798829 | 0.38798829 | loss | 0.854797625 | 0.466809335 | NTNG2 | 37 | 9 |
| cg16726706 | 7.13E−10 | 3.03E−04 | −0.387808799 | 0.387808799 | loss | 0.917478831 | 0.529670082 | | 37 | 12 |
| cg12883279 | 7.13E−10 | 3.03E−04 | −0.387678531 | 0.387678531 | loss | 0.891217472 | 0.503538941 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg04149015 | 7.13E−10 | 3.03E−04 | −0.387478936 | 0.387478936 | loss | 0.625001594 | 0.237522659 | PRSS36; PRSS36; PRSS36 | 37 | 16 |
| cg00307818 | 7.13E−10 | 3.03E−04 | −0.387468199 | 0.387468199 | loss | 0.827268881 | 0.439800682 | BSND | 37 | 1 |
| cg01191259 | 9.23E−10 | 3.92E−04 | −0.387266798 | 0.387266798 | loss | 0.794749862 | 0.407483065 | | 37 | 15 |
| cg04196143 | 7.12E−10 | 3.02E−04 | −0.387172999 | 0.387172999 | loss | 0.94481597 | 0.557642971 | | 37 | 2 |
| cg23381646 | 7.13E−10 | 3.03E−04 | −0.38694345 | 0.38694345 | loss | 0.720118785 | 0.333175335 | GCK; GCK; GCK | 37 | 7 |
| cg05752349 | 2.16E−09 | 9.17E−04 | −0.38659714 | 0.38659714 | loss | 0.77065597 | 0.384058829 | | 37 | 1 |
| cg20368818 | 1.30E−09 | 5.52E−04 | −0.386530616 | 0.386530616 | loss | 0.721905604 | 0.335374988 | | 37 | 11 |
| cg26874229 | 1.10E−09 | 4.65E−04 | −0.386410334 | 0.386410334 | loss | 0.561446706 | 0.175036372 | | 37 | 2 |
| cg05656990 | 7.13E−10 | 3.03E−04 | −0.38640058 | 0.38640058 | loss | 0.46400084 | 0.07760026 | | 37 | 13 |
| cg01803461 | 7.13E−10 | 3.03E−04 | −0.386341997 | 0.386341997 | loss | 0.875956015 | 0.489614018 | | 37 | 16 |
| cg12208770 | 7.13E−10 | 3.03E−04 | −0.386286134 | 0.386286134 | loss | 0.590839675 | 0.204553541 | TMEM88B | 37 | 1 |
| cg10730435 | 7.77E−10 | 3.30E−04 | −0.386164812 | 0.386164812 | loss | 0.6275964 | 0.241431588 | | 37 | 15 |
| cg23652484 | 7.13E−10 | 3.03E−04 | −0.385903725 | 0.385903725 | loss | 0.715465425 | 0.3295617 | | 37 | 1 |
| cg04478991 | 7.13E−10 | 3.03E−04 | −0.385899172 | 0.385899172 | loss | 0.705004102 | 0.319104929 | SLC51A | 37 | 3 |
| cg16151651 | 7.77E−10 | 3.30E−04 | −0.385868339 | 0.385868339 | loss | 0.583413983 | 0.197545644 | | 37 | 2 |
| cg19566586 | 7.13E−10 | 3.03E−04 | −0.385730709 | 0.385730709 | loss | 0.772111409 | 0.3863807 | TRIM7; TRIM7; TRIM7; TRIM7; | 37 | 5 |
| | | | | | | | | TRIM7 | | |
| cg19185846 | 7.13E−10 | 3.03E−04 | −0.38568996 | 0.38568996 | loss | 0.61652376 | 0.2308338 | | 37 | 2 |
| cg01971181 | 7.13E−10 | 3.03E−04 | −0.38542176 | 0.38542176 | loss | 0.720818277 | 0.335396518 | MF5D1; MFSD1 | 37 | 3 |
| cg05189127 | 7.13E−10 | 3.03E−04 | −0.385330271 | 0.385330271 | loss | 0.608563483 | 0.223233212 | | 37 | 10 |
| cg17598339 | 1.99E−09 | 8.43E−04 | −0.385313974 | 0.385313974 | loss | 0.765541515 | 0.380227541 | | 37 | 2 |
| cg25426302 | 7.13E−10 | 3.03E−04 | −0.385280738 | 0.385280738 | loss | 0.905147991 | 0.519867253 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg06640630 | 7.13E−10 | 3.03E−04 | −0.385187333 | 0.385187333 | loss | 0.623039051 | 0.237851718 | RD3; RD3 | 37 | 1 |
| cg09407967 | 7.13E−10 | 3.03E−04 | −0.385183582 | 0.385183582 | loss | 0.7442517 | 0.359068118 | | 37 | 6 |
| cg23669081 | 7.36E−08 | 3.12E−02 | −0.385170345 | 0.385170345 | loss | 0.60440385 | 0.219233505 | HOXB7 | 37 | 17 |
| cg23458168 | 7.13E−10 | 3.03E−04 | −0.38500903 | 0.38500903 | loss | 0.58339673 | 0.1983877 | ZNF536 | 37 | 19 |
| cg07355189 | 1.30E−09 | 5.52E−04 | −0.384990746 | 0.384990746 | loss | 0.874922687 | 0.489931941 | ALOX5; ALOX5; ALOX5 | 37 | 10 |
| cg20167074 | 1.10E−09 | 4.65E−04 | −0.384909004 | 0.384909004 | loss | 0.637656198 | 0.252747194 | S100A10 | 37 | 1 |
| cg21490444 | 7.13E−10 | 3.03E−04 | −0.384906943 | 0.384906943 | loss | 0.973911708 | 0.589004765 | | 37 | 5 |
| cg09285851 | 1.42E−09 | 6.01E−04 | −0.384834122 | 0.384834122 | loss | 0.786490328 | 0.401656206 | | 37 | 6 |
| cg01874730 | 7.84E−10 | 3.33E−04 | −0.384819865 | 0.384819865 | loss | 0.737781865 | 0.352962 | CX3CL1 | 37 | 16 |
| cg05771342 | 7.13E−10 | 3.03E−04 | −0.384587868 | 0.384587868 | loss | 0.727649121 | 0.343061253 | MRGPRF; MRGPRF | 37 | 11 |
| cg22953865 | 7.13E−10 | 3.03E−04 | −0.384477606 | 0.384477606 | loss | 0.8838697 | 0.499392094 | CCDC85C | 37 | 14 |
| cg04058281 | 7.13E−10 | 3.03E−04 | −0.384380544 | 0.384380544 | loss | 0.872405138 | 0.488024594 | | 37 | 5 |
| cg24729928 | 1.68E−09 | 7.12E−04 | −0.384338217 | 0.384338217 | loss | 0.666735864 | 0.282397647 | FAM60A; FAM60A; FAM60A | 37 | 12 |
| cg04154027 | 8.47E−10 | 3.60E−04 | −0.384275558 | 0.384275558 | loss | 0.799826104 | 0.415550546 | CMYA5 | 37 | 5 |
| cg14875327 | 7.13E−10 | 3.03E−04 | −0.384200927 | 0.384200927 | loss | 0.915243351 | 0.531042424 | | 37 | 3 |
| cg01826254 | 7.13E−10 | 3.03E−04 | −0.384151972 | 0.384151972 | loss | 0.755898813 | 0.371746841 | | 37 | 16 |
| cg06307176 | 7.13E−10 | 3.03E−04 | −0.383952402 | 0.383952402 | loss | 0.902915238 | 0.518962835 | LOC728637; LOC728637 | 37 | 5 |
| cg06320582 | 8.47E−10 | 3.60E−04 | −0.38393741 | 0.38393741 | loss | 0.879032887 | 0.495095476 | | 37 | 16 |
| cg16501323 | 7.13E−10 | 3.03E−04 | −0.383879924 | 0.383879924 | loss | 0.59285953 | 0.208979606 | AIRE | 37 | 21 |
| cg22972858 | 1.68E−09 | 7.12E−04 | −0.383829106 | 0.383829106 | loss | 0.905429806 | 0.5216007 | HCAR1; HCAR1 | 37 | 12 |
| cg20055237 | 7.13E−10 | 3.03E−04 | −0.383821682 | 0.383821682 | loss | 0.514517515 | 0.130695834 | | 37 | 2 |
| cg20638675 | 7.13E−10 | 3.03E−04 | −0.383554623 | 0.383554623 | loss | 0.411952743 | 0.02839812 | TMEM72-AS1 | 37 | 10 |
| cg20893180 | 9.23E−10 | 3.92E−04 | −0.383431542 | 0.383431542 | loss | 0.647831236 | 0.264399694 | | 37 | 8 |
| cg06294700 | 7.13E−10 | 3.03E−04 | −0.383390922 | 0.383390922 | loss | 0.845368981 | 0.461978059 | | 37 | 5 |
| cg14389669 | 7.13E−10 | 3.03E−04 | −0.383212855 | 0.383212855 | loss | 0.44241513 | 0.059202275 | ADAMTSL2; ADAMTSL2 | 37 | 9 |
| cg20091636 | 2.16E−09 | 9.17E−04 | −0.383047674 | 0.383047674 | loss | 0.632173115 | 0.249125441 | LOC100505532; LOC100505532 | 37 | 8 |
| cg04387835 | 7.77E−10 | 3.30E−04 | −0.382999486 | 0.382999486 | loss | 0.48003636 | 0.097036874 | ZMYND15; ZMYND15; ZMYND15 | 37 | 17 |
| cg26939946 | 7.13E−10 | 3.03E−04 | −0.382981509 | 0.382981509 | loss | 0.858299709 | 0.4753182 | RNF112 | 37 | 17 |
| cg14677130 | 5.39E−09 | 2.29E−03 | −0.382701449 | 0.382701449 | loss | 0.93442846 | 0.551727012 | EGFLAM; EGFLAM; EGFLAM; | 37 | 5 |
| | | | | | | | | EGFLAM | | |
| cg27359374 | 7.13E−10 | 3.03E−04 | −0.382627347 | 0.382627347 | loss | 0.957584864 | 0.574957518 | | 37 | 6 |
| cg04589210 | 7.13E−10 | 3.03E−04 | −0.382404732 | 0.382404732 | loss | 0.643305074 | 0.260900341 | | 37 | 2 |
| cg14035368 | 9.23E−10 | 3.92E−04 | −0.3823009 | 0.3823009 | loss | 0.731264442 | 0.348963541 | DNLZ; CARD9; CARD9; DNLZ | 37 | 9 |
| cg21160842 | 7.13E−10 | 3.03E−04 | −0.382184848 | 0.382184848 | loss | 0.711226636 | 0.329041788 | RCN1 | 37 | 11 |
| cg23058037 | 7.84E−10 | 3.33E−04 | −0.382161884 | 0.382161884 | loss | 0.605869819 | 0.223707935 | VIPR2 | 37 | 7 |
| cg15491131 | 7.13E−10 | 3.03E−04 | −0.382154694 | 0.382154694 | loss | 0.694593342 | 0.312438647 | | 37 | 13 |
| cg09088406 | 7.77E−10 | 3.30E−04 | −0.382087497 | 0.382087497 | loss | 0.847601068 | 0.465513571 | | 37 | 15 |
| cg24698211 | 7.13E−10 | 3.03E−04 | −0.38204201 | 0.38204201 | loss | 0.606203345 | 0.224161335 | FGF18 | 37 | 5 |
| cg16926213 | 7.13E−10 | 3.03E−04 | −0.382001079 | 0.382001079 | loss | 0.826119726 | 0.444118647 | | 37 | 1 |
| cg12082025 | 7.84E−10 | 3.33E−04 | −0.381930912 | 0.381930912 | loss | 0.949895794 | 0.567964882 | ABCA7 | 37 | 19 |
| cg24280832 | 7.13E−10 | 3.03E−04 | −0.381826352 | 0.381826352 | loss | 0.915798428 | 0.533972076 | C10orf11 | 37 | 10 |
| cg11801727 | 7.13E−10 | 3.03E−04 | −0.38161381 | 0.38161381 | loss | 0.840545351 | 0.458931541 | | 37 | 1 |
| cg04270401 | 7.13E−10 | 3.03E−04 | −0.381419678 | 0.381419678 | loss | 0.814129819 | 0.432710141 | | 37 | 10 |
| cg17611235 | 9.23E−10 | 3.92E−04 | −0.381284181 | 0.381284181 | loss | 0.684289334 | 0.303005153 | | 37 | 11 |
| cg20201270 | 1.67E−08 | 7.10E−03 | −0.381270391 | 0.381270391 | loss | 0.524935813 | 0.143665422 | | 37 | 2 |
| cg03704673 | 7.13E−10 | 3.03E−04 | −0.381247236 | 0.381247236 | loss | 0.757594325 | 0.376347088 | GLTPD2 | 37 | 17 |
| cg11027707 | 7.13E−10 | 3.03E−04 | −0.380962572 | 0.380962572 | loss | 0.826633602 | 0.445671029 | MON1A; MON1A | 37 | 3 |
| cg05527459 | 7.13E−10 | 3.03E−04 | −0.380854794 | 0.380854794 | loss | 0.905412047 | 0.524557253 | | 37 | 1 |
| cg23389215 | 7.13E−10 | 3.03E−04 | −0.380801266 | 0.380801266 | loss | 0.65894646 | 0.278145194 | NRXN2; NRXN2; NRXN2 | 37 | 11 |
| cg20035032 | 7.13E−10 | 3.03E−04 | −0.380778804 | 0.380778804 | loss | 0.80865814 | 0.427879335 | | 37 | 17 |
| cg07290269 | 1.19E−09 | 5.07E−04 | −0.380722552 | 0.380722552 | loss | 0.903068628 | 0.522346076 | FAM60A; FAM60A; FAM60A | 37 | 12 |
| cg14711997 | 7.13E−10 | 3.03E−04 | −0.380651681 | 0.380651681 | loss | 0.517199168 | 0.136547486 | KCNH2; KCNH2; KCNH2; KCNH2; | 37 | 7 |
| | | | | | | | | KCNH2; KCNH2 | | |
| cg19299265 | 1.01E−09 | 4.27E−04 | −0.380635448 | 0.380635448 | loss | 0.927530036 | 0.546894588 | | 37 | 10 |
| cg00426822 | 7.13E−10 | 3.03E−04 | −0.380625245 | 0.380625245 | loss | 0.589402715 | 0.208777471 | | 37 | 1 |
| cg21587238 | 2.96E−09 | 1.26E−03 | −0.380563682 | 0.380563682 | loss | 0.664596725 | 0.284033044 | | 37 | 5 |
| cg13474370 | 7.13E−10 | 3.03E−04 | −0.380382349 | 0.380382349 | loss | 0.576178672 | 0.195796323 | RBPMS2 | 37 | 15 |
| cg10282491 | 7.13E−10 | 3.03E−04 | −0.380317925 | 0.380317925 | loss | 0.695284413 | 0.314966488 | CLDN9 | 37 | 16 |
| cg14971320 | 7.13E−10 | 3.03E−04 | −0.380310416 | 0.380310416 | loss | 0.484951389 | 0.104640973 | RASGEF1A; RASGEF1A | 37 | 10 |
| cg21969792 | 7.13E−10 | 3.03E−04 | −0.380150185 | 0.380150185 | loss | 0.853596226 | 0.473446041 | | 37 | 4 |
| cg05150608 | 7.13E−10 | 3.03E−04 | −0.379974545 | 0.379974545 | loss | 0.533387157 | 0.153412612 | | 37 | 14 |
| cg08090164 | 7.84E−10 | 3.33E−04 | −0.379957575 | 0.379957575 | loss | 0.812875981 | 0.432918406 | LYNX1; LYNX1; LYNX1; LYNX1; | 37 | 8 |
| | | | | | | | | LYNX1; LYNX1 | | |
| cg10444806 | 7.13E−10 | 3.03E−04 | −0.37992765 | 0.37992765 | loss | 0.539193798 | 0.159266148 | ORMDL3 | 37 | 17 |
| cg06618740 | 7.13E−10 | 3.03E−04 | −0.379885016 | 0.379885016 | loss | 0.750232581 | 0.370347565 | | 37 | 1 |
| cg15282281 | 7.13E−10 | 3.03E−04 | −0.379879949 | 0.379879949 | loss | 0.640892449 | 0.2610125 | GRID1; MIR346; MIR346 | 37 | 10 |
| cg13187827 | 9.22E−08 | 3.91E−02 | −0.37985762 | 0.37985762 | loss | 0.670116843 | 0.290259224 | VWA7 | 37 | 6 |
| cg10932486 | 1.10E−09 | 4.65E−04 | −0.379445644 | 0.379445644 | loss | 0.596918542 | 0.217472898 | | 37 | 5 |
| cg15090036 | 8.47E−10 | 3.60E−04 | −0.379381397 | 0.379381397 | loss | 0.844178832 | 0.464797435 | | 37 | 17 |
| cg01057945 | 7.13E−10 | 3.03E−04 | −0.37936667 | 0.37936667 | loss | 0.786901858 | 0.407535188 | LOC283332 | 37 | 12 |
| cg10022914 | 7.13E−10 | 3.03E−04 | −0.379322975 | 0.379322975 | loss | 0.72590584 | 0.346582865 | | 37 | 10 |
| cg10321156 | 7.13E−10 | 3.03E−04 | −0.379259569 | 0.379259569 | loss | 0.433236853 | 0.053977284 | | 37 | 11 |
| cg14381677 | 7.13E−10 | 3.03E−04 | −0.379069545 | 0.379069545 | loss | 0.873552462 | 0.494482918 | KDM5B; PCAT6; PCAT6 | 37 | 1 |
| cg22171758 | 7.13E−10 | 3.03E−04 | −0.378980534 | 0.378980534 | loss | 0.809339575 | 0.430359041 | CISTR-ACT; CISTR-ACT | 37 | 12 |
| cg22657492 | 7.13E−10 | 3.03E−04 | −0.378958242 | 0.378958242 | loss | 0.964750783 | 0.585792541 | GFRA3 | 37 | 5 |
| cg15234946 | 7.13E−10 | 3.03E−04 | −0.378925048 | 0.378925048 | loss | 0.470568904 | 0.091643856 | | 37 | 6 |
| cg07264025 | 1.42E−09 | 6.01E−04 | −0.378783381 | 0.378783381 | loss | 0.683988245 | 0.305204865 | MGAT4C | 37 | 12 |
| cg13451356 | 7.13E−10 | 3.03E−04 | −0.378777463 | 0.378777463 | loss | 0.553632415 | 0.174854952 | | 37 | 1 |
| cg25701444 | 7.13E−10 | 3.03E−04 | −0.378661877 | 0.378661877 | loss | 0.738430489 | 0.359768612 | LOC400043 | 37 | 12 |
| cg16205352 | 7.13E−10 | 3.03E−04 | −0.378582848 | 0.378582848 | loss | 0.722989677 | 0.344406829 | PRPH | 37 | 12 |
| cg02845274 | 9.23E−10 | 3.92E−04 | −0.378573251 | 0.378573251 | loss | 0.924972904 | 0.546399653 | ABCA13 | 37 | 7 |
| cg23243408 | 7.13E−10 | 3.03E−04 | −0.378396664 | 0.378396664 | loss | 0.873195617 | 0.494798953 | | 37 | 2 |
| cg13136220 | 7.13E−10 | 3.03E−04 | −0.378385561 | 0.378385561 | loss | 0.770072443 | 0.391686882 | LOC100287846 | 37 | 8 |
| cg23444264 | 7.13E−10 | 3.03E−04 | −0.378371379 | 0.378371379 | loss | 0.723936338 | 0.345564959 | | 37 | 10 |
| cg01015662 | 7.77E−10 | 3.30E−04 | −0.37828759 | 0.37828759 | loss | 0.911869713 | 0.533582124 | | 37 | 1 |
| cg18877667 | 7.13E−10 | 3.03E−04 | −0.378126465 | 0.378126465 | loss | 0.643081789 | 0.264955324 | DLX4; DLX4 | 37 | 17 |
| cg02797195 | 7.13E−10 | 3.03E−04 | −0.378083321 | 0.378083321 | loss | 0.778993504 | 0.400910182 | RIPK4 | 37 | 21 |
| cg06939851 | 7.13E−10 | 3.03E−04 | −0.37776504 | 0.37776504 | loss | 0.54865126 | 0.17088622 | PYCR2; PYCR2 | 37 | 1 |
| cg27152312 | 2.69E−08 | 1.14E−02 | −0.377679012 | 0.377679012 | loss | 0.689784906 | 0.312105894 | | 37 | 7 |
| cg24361265 | 1.73E−09 | 7.34E−04 | −0.377623706 | 0.377623706 | loss | 0.779992857 | 0.402369151 | SERF2; ELL3 | 37 | 15 |
| cg00049595 | 7.13E−10 | 3.03E−04 | −0.377545075 | 0.377545075 | loss | 0.52650594 | 0.148960865 | | 37 | 6 |
| cg24980657 | 7.13E−10 | 3.03E−04 | −0.377410405 | 0.377410405 | loss | 0.571341336 | 0.193930931 | | 37 | 2 |
| cg23683010 | 1.87E−09 | 7.93E−04 | −0.377386854 | 0.377386854 | loss | 0.929982642 | 0.552595788 | MRGPRE | 37 | 11 |
| cg17158913 | 7.13E−10 | 3.03E−04 | −0.377257181 | 0.377257181 | loss | 0.816483975 | 0.439226794 | CASZ1; CASZ1 | 37 | 1 |
| cg04797061 | 7.13E−10 | 3.03E−04 | −0.377132201 | 0.377132201 | loss | 0.71383193 | 0.336699729 | PRSS36; PRSS36; PRSS36 | 37 | 16 |
| cg24025550 | 7.13E−10 | 3.03E−04 | −0.376824925 | 0.376824925 | loss | 0.408860475 | 0.032035551 | | 37 | 15 |
| cg05131488 | 7.13E−10 | 3.03E−04 | −0.376818673 | 0.376818673 | loss | 0.820451949 | 0.443633276 | ETV1; ETV1 | 37 | 7 |
| cg13521002 | 7.13E−10 | 3.03E−04 | −0.376777216 | 0.376777216 | loss | 0.881522698 | 0.504745482 | PEBP4 | 37 | 8 |
| cg25862444 | 7.13E−10 | 3.03E−04 | −0.376759674 | 0.376759674 | loss | 0.634028255 | 0.257268581 | NBPF8; NBPF8; NBPF8 | 37 | 1 |
| cg27283514 | 7.77E−10 | 3.30E−04 | −0.376603769 | 0.376603769 | loss | 0.690798281 | 0.314194512 | | 37 | 16 |
| cg00780971 | 1.30E−09 | 5.52E−04 | 0.376364842 | 0.376364842 | gain | 0.176655964 | 0.553020806 | | 37 | 1 |
| cg01515960 | 7.13E−10 | 3.03E−04 | −0.376333748 | 0.376333748 | loss | 0.61742293 | 0.241089182 | | 37 | 19 |
| cg23038338 | 7.13E−10 | 3.03E−04 | −0.376211254 | 0.376211254 | loss | 0.672606413 | 0.296395159 | | 37 | 1 |
| cg01162093 | 7.13E−10 | 3.03E−04 | −0.376166707 | 0.376166707 | loss | 0.741945866 | 0.365779159 | | 37 | 10 |
| cg02992639 | 7.13E−10 | 3.03E−04 | −0.376160266 | 0.376160266 | loss | 0.5144742 | 0.138313934 | | 37 | 1 |
| cg06136160 | 7.13E−10 | 3.03E−04 | −0.376145802 | 0.376145802 | loss | 0.826207191 | 0.450061388 | PACRG; PACRG; PACRG | 37 | 6 |
| cg01271726 | 7.13E−10 | 3.03E−04 | −0.375996954 | 0.375996954 | loss | 0.873803883 | 0.497811929 | | 37 | 6 |
| cg19021188 | 1.10E−09 | 4.65E−04 | −0.375971734 | 0.375971734 | loss | 0.736496334 | 0.3605246 | LYNX1; LYNX1; LYNX1; LYNX1; | 37 | 8 |
| | | | | | | | | LYNX1; LYNX1 | | |
| cg16674433 | 7.13E−10 | 3.03E−04 | −0.37590365 | 0.37590365 | loss | 0.641586715 | 0.265683065 | RNF112 | 37 | 17 |
| cg12262698 | 7.13E−10 | 3.03E−04 | −0.375887307 | 0.375887307 | loss | 0.612166025 | 0.236278718 | FAM118A; FAM118A | 37 | 22 |
| cg03954144 | 7.77E−10 | 3.30E−04 | −0.375667246 | 0.375667246 | loss | 0.548492926 | 0.17282568 | | 37 | 13 |
| cg00433296 | 7.13E−10 | 3.03E−04 | −0.375655711 | 0.375655711 | loss | 0.752544011 | 0.3768883 | SLC39A14; SLC39A14; SLC39A14; | 37 | 8 |
| | | | | | | | | SLC39A14 | | |
| cg14635466 | 7.13E−10 | 3.03E−04 | −0.375619121 | 0.375619121 | loss | 0.608505058 | 0.232885938 | SNX31 | 37 | 8 |
| cg23005885 | 7.13E−10 | 3.03E−04 | −0.375607822 | 0.375607822 | loss | 0.964136151 | 0.588528329 | ZNF710 | 37 | 15 |
| cg21049587 | 7.13E−10 | 3.03E−04 | −0.375552101 | 0.375552101 | loss | 0.799604336 | 0.424052235 | RGN; RGN; RGN; RGN | 37 | X |
| cg19041857 | 1.09E−08 | 4.62E−03 | −0.375438205 | 0.375438205 | loss | 0.8627694 | 0.487331195 | LOC100131289 | 37 | 6 |
| cg19529709 | 1.19E−09 | 5.07E−04 | −0.375245163 | 0.375245163 | loss | 0.913396634 | 0.538151471 | PIWIL3; PIWIL3; TOP1P2; PIWIL3; | | |
| | | | | | | | | PIWIL3 | 37 | 22 |
| cg23080060 | 4.57E−09 | 1.94E−03 | −0.375211693 | 0.375211693 | loss | 0.65835654 | 0.283144847 | | 37 | 5 |
| cg00424451 | 7.13E−10 | 3.03E−04 | −0.375189101 | 0.375189101 | loss | 0.605829136 | 0.230640035 | | 37 | 17 |
| cg07786675 | 7.13E−10 | 3.03E−04 | −0.375186541 | 0.375186541 | loss | 0.926167858 | 0.550981318 | SFN | 37 | 1 |
| cg05310240 | 7.13E−10 | 3.03E−04 | −0.375142107 | 0.375142107 | loss | 0.684690777 | 0.309548671 | | 37 | 17 |
| cg13419925 | 7.13E−10 | 3.03E−04 | −0.375054601 | 0.375054601 | loss | 0.694221519 | 0.319166918 | MFSD1; MFSD1 | 37 | 3 |
| cg03329019 | 2.26E−09 | 9.62E−04 | −0.374915379 | 0.374915379 | loss | 0.754999966 | 0.380084588 | | 37 | 1 |
| cg12165772 | 7.13E−10 | 3.03E−04 | −0.374898389 | 0.374898389 | loss | 0.57847853 | 0.203580141 | DLX3 | 37 | 17 |
| cg24346776 | 7.13E−10 | 3.03E−04 | −0.374894997 | 0.374894997 | loss | 0.679823136 | 0.304928139 | NBPF8; NBPF8; NBPF8 | 37 | 1 |
| cg11750580 | 7.84E−10 | 3.33E−04 | −0.374885999 | 0.374885999 | loss | 0.878981635 | 0.504095635 | CCDC17; CCDC17 | 37 | 1 |
| cg20768358 | 7.13E−10 | 3.03E−04 | −0.374831434 | 0.374831434 | loss | 0.600182892 | 0.225351459 | TP73; TP73; TP73; TP73; TP73; | 37 | 1 |
| | | | | | | | | TP73 | | |
| cg12080906 | 7.13E−10 | 3.03E−04 | −0.374816489 | 0.374816489 | loss | 0.737646342 | 0.362829853 | NES | 37 | 1 |
| cg20395040 | 7.13E−10 | 3.03E−04 | −0.374758805 | 0.374758805 | loss | 0.699839028 | 0.325080224 | | 37 | 13 |
| cg20660989 | 7.13E−10 | 3.03E−04 | −0.374540152 | 0.374540152 | loss | 0.70774447 | 0.333204318 | | 37 | 6 |
| cg17542176 | 7.13E−10 | 3.03E−04 | −0.374412219 | 0.374412219 | loss | 0.681947508 | 0.307535288 | | 37 | X |
| cg19401111 | 7.13E−10 | 3.03E−04 | −0.374200248 | 0.374200248 | loss | 0.711699819 | 0.337499571 | SLC51A | 37 | 3 |
| cg06853455 | 7.13E−10 | 3.03E−04 | −0.374053133 | 0.374053133 | loss | 0.819477962 | 0.445424829 | | 37 | 12 |
| cg08269485 | 7.13E−10 | 3.03E−04 | −0.373863698 | 0.373863698 | loss | 0.604223002 | 0.230359304 | IFITM10; MOB2 | 37 | 11 |
| cg26633084 | 7.84E−10 | 3.33E−04 | −0.373844945 | 0.373844945 | loss | 0.84474601 | 0.470901065 | | 37 | 4 |
| cg07318204 | 9.54E−09 | 4.05E−03 | −0.373837561 | 0.373837561 | loss | 0.765063043 | 0.391225482 | HHIP; HHIP-AS1 | 37 | 4 |
| cg26675395 | 7.13E−10 | 3.03E−04 | −0.373686526 | 0.373686526 | loss | 0.512691732 | 0.139005206 | PRSS36; PRSS36; PRSS36 | 37 | 16 |
| cg03136493 | 9.23E−10 | 3.92E−04 | −0.37368387 | 0.37368387 | loss | 0.73461127 | 0.3609274 | | 37 | 6 |
| cg04066637 | 7.13E−10 | 3.03E−04 | −0.373683678 | 0.373683678 | loss | 0.669975408 | 0.296292729 | TMEM114 | 37 | 16 |
| cg18026197 | 3.28E−09 | 1.39E−03 | −0.373587232 | 0.373587232 | loss | 0.680807591 | 0.307220359 | CHCHD6 | 37 | 3 |
| cg14218447 | 7.13E−10 | 3.03E−04 | −0.373552568 | 0.373552568 | loss | 0.496705257 | 0.123152688 | | 37 | 3 |
| cg05190790 | 7.84E−10 | 3.33E−04 | −0.373502427 | 0.373502427 | loss | 0.654390298 | 0.280887871 | | 37 | 11 |
| cg13818316 | 7.13E−10 | 3.03E−04 | −0.373489788 | 0.373489788 | loss | 0.804703347 | 0.431213559 | RIPK4 | 37 | 21 |
| cg16060369 | 7.13E−10 | 3.03E−04 | −0.373245625 | 0.373245625 | loss | 0.62639816 | 0.253152535 | RASGEF1A; RASGEF1A | 37 | 10 |
| cg11324650 | 7.13E−10 | 3.03E−04 | −0.373137926 | 0.373137926 | loss | 0.513091326 | 0.139953401 | | 37 | 3 |
| cg21287054 | 7.13E−10 | 3.03E−04 | −0.373070232 | 0.373070232 | loss | 0.500371083 | 0.127300851 | TTYH1; TTYH1; TTYH1 | 37 | 19 |
| cg02770672 | 8.47E−10 | 3.60E−04 | −0.372862643 | 0.372862643 | loss | 0.879485343 | 0.5066227 | | 37 | 6 |
| cg15122439 | 2.12E−08 | 9.01E−03 | −0.372692004 | 0.372692004 | loss | 0.828939651 | 0.456247647 | | 37 | 2 |
| cg14074251 | 7.13E−10 | 3.03E−04 | −0.372596697 | 0.372596697 | loss | 0.739831674 | 0.367234976 | SPEG | 37 | 2 |
| cg22532774 | 7.13E−10 | 3.03E−04 | −0.372588677 | 0.372588677 | loss | 0.773722525 | 0.401133847 | ESPNL | 37 | 2 |
| cg02959988 | 1.73E−09 | 7.34E−04 | −0.372571105 | 0.372571105 | loss | 0.859217368 | 0.486646263 | | 37 | 1 |
| cg03455316 | 7.13E−10 | 3.03E−04 | −0.372362057 | 0.372352057 | loss | 0.632445585 | 0.260083528 | | 37 | 15 |
| cg16837557 | 8.47E−10 | 3.60E−04 | −0.372266591 | 0.372266591 | loss | 0.802267674 | 0.430001082 | KIRREL3; KIRREL3 | 37 | 11 |
| cg09517766 | 2.16E−09 | 9.17E−04 | −0.372222234 | 0.372222234 | loss | 0.664842687 | 0.292620453 | | 37 | 10 |
| cg27516159 | 7.13E−10 | 3.03E−04 | −0.372180534 | 0.372180534 | loss | 0.703459687 | 0.331289153 | CACNA2D4 | 37 | 12 |
| cg05565052 | 8.47E−10 | 3.60E−04 | −0.372152038 | 0.372152038 | loss | 0.695222485 | 0.323070447 | TMEM184B; TMEM184B; | 37 | 22 |
| | | | | | | | | TMEM184B | | |
| cg13306087 | 7.13E−10 | 3.03E−04 | −0.37167472 | 0.37167472 | loss | 0.770482891 | 0.398808171 | | 37 | 5 |
| cg00870514 | 7.13E−10 | 3.03E−04 | −0.37157935 | 0.37157935 | loss | 0.726967068 | 0.355387718 | RAB11FIP4 | 37 | 17 |
| cg05090759 | 1.32E−08 | 5.59E−03 | −0.371530593 | 0.371530593 | loss | 0.684084081 | 0.312553488 | SLC38A11; SLC38A11; SLC38A11; | 37 | 2 |
| | | | | | | | | SLC36A11 | | |
| cg24176311 | 7.13E−10 | 3.03E−04 | −0.371476747 | 0.371476747 | loss | 0.910779294 | 0.539302547 | TNPO3; TNPO3; TPI1P2; TNPO3 | 37 | 7 |
| cg13837679 | 7.13E−10 | 3.03E−04 | −0.371466216 | 0.371466216 | loss | 0.694672087 | 0.323205871 | ALX4 | 37 | 11 |
| cg01587386 | 1.19E−09 | 5.07E−04 | −0.371357815 | 0.371357815 | loss | 0.803929615 | 0.4325718 | | 37 | 7 |
| cg00816387 | 7.13E−10 | 3.03E−04 | −0.371173161 | 0.371173161 | loss | 0.896750149 | 0.525576988 | | 37 | 10 |
| cg03110795 | 7.13E−10 | 3.03E−04 | −0.371171427 | 0.371171427 | loss | 0.666253021 | 0.295081594 | | 37 | 7 |
| cg15670924 | 1.01E−09 | 4.27E−04 | −0.37116692 | 0.37116692 | loss | 0.6026219 | 0.23145498 | DNAJB13 | 37 | 11 |
| cg03222009 | 7.13E−10 | 3.03E−04 | −0.371018877 | 0.371018877 | loss | 0.713009342 | 0.341990465 | HS6ST1 | 37 | 2 |
| cg16330552 | 5.85E−09 | 2.49E−03 | −0.370788391 | 0.370788391 | loss | 0.854454532 | 0.483666141 | | 37 | 3 |
| cg04457658 | 7.77E−10 | 3.30E−04 | −0.37070869 | 0.37070869 | loss | 0.678673455 | 0.307964765 | | 37 | 6 |
| cg02954987 | 7.13E−10 | 3.03E−04 | −0.370543317 | 0.370543317 | loss | 0.58683277 | 0.216289453 | LAMB2; LAMB2 | 37 | 3 |
| cg13466284 | 7.13E−10 | 3.03E−04 | −0.370392751 | 0.370392751 | loss | 0.840196145 | 0.469803394 | SFN | 37 | 1 |
| cg01628467 | 7.13E−10 | 3.03E−04 | −0.370235993 | 0.370235993 | loss | 0.969170234 | 0.598934241 | | 37 | 17 |
| cg05092353 | 7.13E−10 | 3.03E−04 | −0.370172944 | 0.370172944 | loss | 0.696929685 | 0.326756741 | F10 | 37 | 13 |
| cg03743678 | 7.13E−10 | 3.03E−04 | −0.37010889 | 0.37010889 | loss | 0.955266096 | 0.585157206 | | 37 | 16 |
| cg13001878 | 7.13E−10 | 3.03E−04 | −0.37009136 | 0.37009136 | loss | 0.767573166 | 0.397481806 | F10 | 37 | 13 |
| cg03287432 | 7.13E−10 | 3.03E−04 | −0.370084575 | 0.370084575 | loss | 0.698818945 | 0.328734371 | | 37 | 10 |
| cg22400703 | 7.13E−10 | 3.03E−04 | −0.370075566 | 0.370075566 | loss | 0.667196808 | 0.297121241 | CNRIP1; CNRIP1 | 37 | 2 |
| cg27637303 | 1.68E−09 | 7.12E−04 | −0.37004343 | 0.37004343 | loss | 0.645123736 | 0.275080306 | | 37 | 2 |
| cg07142285 | 7.13E−10 | 3.03E−04 | −0.370017882 | 0.370017882 | loss | 0.893841206 | 0.523823324 | APCDD1L | 37 | 20 |
| cg11942181 | 7.13E−10 | 3.03E−04 | −0.369979906 | 0.369979906 | loss | 0.515024894 | 0.145044988 | | 37 | 2 |
| cg08515989 | 7.13E−10 | 3.03E−04 | −0.369954179 | 0.369954179 | loss | 0.926498191 | 0.556544012 | ASPHD1 | 37 | 16 |
| cg01412970 | 3.18E−09 | 1.35E−03 | −0.369822809 | 0.369822809 | loss | 0.413986432 | 0.044163623 | PLD6 | 37 | 17 |
| cg13170076 | 7.13E−10 | 3.03E−04 | −0.36981752 | 0.36981752 | loss | 0.691463591 | 0.321646071 | | 37 | 2 |
| cg07743117 | 7.13E−10 | 3.03E−04 | −0.3698001 | 0.3698001 | loss | 0.9339333 | 0.5641332 | | 37 | 5 |
| cg14659662 | 1.32E−08 | 5.59E−03 | −0.369750211 | 0.369750211 | loss | 0.788138464 | 0.418388253 | GLIS1 | 37 | 1 |
| cg19573236 | 7.13E−10 | 3.03E−04 | −0.369687799 | 0.369587799 | loss | 0.845792075 | 0.476104276 | | 37 | 10 |
| cg04134279 | 7.13E−10 | 3.03E−04 | −0.369616122 | 0.369616122 | loss | 0.516184872 | 0.14656875 | TDRP; TDRP | 37 | 8 |
| cg22715761 | 7.13E−10 | 3.03E−04 | −0.369596437 | 0.369596437 | loss | 0.753375219 | 0.383778782 | LDB3; LDB3; LDB3; LDB3; LDB3; | 37 | 10 |
| | | | | | | | | LDB3 | | |
| cg08023416 | 7.13E−10 | 3.03E−04 | −0.369594714 | 0.369594714 | loss | 0.719729055 | 0.350134341 | IL1RL2 | 37 | 2 |
| cg13270055 | 9.23E−10 | 3.92E−04 | −0.369557459 | 0.369557459 | loss | 0.629165289 | 0.259607829 | CACNG2 | 37 | 22 |
| cg00346985 | 7.13E−10 | 3.03E−04 | −0.369400268 | 0.369400268 | loss | 0.509612955 | 0.140212687 | | 37 | 1 |
| cg21903286 | 7.48E−09 | 3.18E−03 | −0.369388075 | 0.369388075 | loss | 0.841073945 | 0.471685871 | PRICKLE1; PRICKLE1; PRICKLE1; | 37 | 12 |
| | | | | | | | | PRICKLE1 | | |
| cg19867917 | 8.47E−10 | 3.60E−04 | −0.369245441 | 0.369245441 | loss | 0.543479868 | 0.174234427 | COLEC11; COLEC11; COLEC11; | 37 | 2 |
| | | | | | | | | COLEC11; COLEC11; COLEC11 | | |
| cg24356964 | 7.13E−10 | 3.03E−04 | −0.369188421 | 0.369188421 | loss | 0.550205821 | 0.1810174 | TP73; TP73; TP73; TP73; TP73; | 37 | 1 |
| | | | | | | | | TP73; TP73; TP73; TP73; | | |
| | | | | | | | | TP73; TP73; TP73 | | |
| cg16893614 | 7.13E−10 | 3.03E−04 | −0.369145322 | 0.369145322 | loss | 0.861970081 | 0.492824759 | ALX3 | 37 | 1 |
| cg02362978 | 7.13E−10 | 3.03E−04 | −0.368984701 | 0.368984701 | loss | 0.861172272 | 0.492187571 | USP43; USP43 | 37 | 17 |
| cg06893273 | 7.13E−10 | 3.03E−04 | −0.368984383 | 0.368984383 | loss | 0.547886125 | 0.178901741 | | 37 | 17 |
| cg17535961 | 7.13E−10 | 3.03E−04 | −0.368501961 | 0.368501961 | loss | 0.898941602 | 0.530439641 | | 37 | 5 |
| cg02952809 | 7.13E−10 | 3.03E−04 | −0.368262161 | 0.368262161 | loss | 0.856956555 | 0.488694394 | HILPDA; HILPDA | 37 | 7 |
| cg11609571 | 7.13E−10 | 3.03E−04 | −0.368148613 | 0.368148613 | loss | 0.513853921 | 0.145705308 | PRELID2; PRELID2; PRELID2 | 37 | 5 |
| cg13680337 | 7.13E−10 | 3.03E−04 | −0.368042004 | 0.368042004 | loss | 0.76182094 | 0.393778935 | COL13A1; COL13A1; COL13A1; | 37 | 10 |
| | | | | | | | | COL13A1; COL13A1; COL13A1 | | |
| cg08575722 | 1.10E−09 | 4.65E−04 | −0.367921559 | 0.367921559 | loss | 0.873807753 | 0.505886194 | WT1; WT1; WT1; WT1; WT1 | 37 | 11 |
| cg22270027 | 1.19E−09 | 5.07E−04 | −0.367855687 | 0.367855687 | loss | 0.77102214 | 0.403165453 | | 37 | 4 |
| cg24830730 | 7.13E−10 | 3.03E−04 | −0.36782306 | 0.36782306 | loss | 0.63129773 | 0.263474671 | KCNH2; KCNH2; KCNH2; KCNH2 | 37 | 7 |
| cg01071644 | 8.47E−10 | 3.60E−04 | −0.367771782 | 0.367771782 | loss | 0.647982853 | 0.280211071 | QSOX2 | 37 | 9 |
| cg04585209 | 9.23E−10 | 3.92E−04 | −0.367743932 | 0.367743932 | loss | 0.811102174 | 0.443358241 | CCKBR | 37 | 11 |
| cg11288801 | 7.13E−10 | 3.03E−04 | −0.367633605 | 0.367633605 | loss | 0.623830934 | 0.256197329 | DAZAP1; DAZAP1 | 37 | 19 |
| cg04204221 | 7.13E−10 | 3.03E−04 | −0.367610782 | 0.367610782 | loss | 0.656080317 | 0.288469535 | | 37 | 2 |
| cg21167269 | 7.77E−10 | 3.30E−04 | −0.367598635 | 0.367598635 | loss | 0.443800051 | 0.076201416 | | 37 | 9 |
| cg04057238 | 7.13E−10 | 3.03E−04 | −0.367582296 | 0.367582296 | loss | 0.950330508 | 0.582748212 | | 37 | 5 |
| cg25433552 | 7.13E−10 | 3.03E−04 | −0.367574999 | 0.367574999 | loss | 0.686877317 | 0.319302318 | | 37 | 5 |
| cg14748380 | 1.10E−09 | 4.65E−04 | −0.367359715 | 0.367359715 | loss | 0.581935956 | 0.214576251 | FAM163A | 37 | 1 |
| cg08710469 | 7.13E−10 | 3.03E−04 | −0.36697585 | 0.36697585 | loss | 0.802918815 | 0.435942965 | | 37 | 1 |
| cg14301190 | 7.13E−10 | 3.03E−04 | −0.366951368 | 0.366951368 | loss | 0.457027502 | 0.090076134 | PRSS36; PRSS36; PRSS36 | 37 | 16 |
| cg08579577 | 1.19E−09 | 5.07E−04 | −0.366941059 | 0.366941059 | loss | 0.536844643 | 0.169903585 | | 37 | X |
| cg24524629 | 7.77E−10 | 3.30E−04 | −0.366869297 | 0.366869297 | loss | 0.638214058 | 0.271344771 | KRTAP2-1; KRTAP2-1 | 37 | 17 |
| cg07045469 | 8.47E−10 | 3.60E−04 | −0.366761624 | 0.366761624 | loss | 0.859216777 | 0.492455153 | ABCA13 | 37 | 7 |
| cg11342437 | 7.13E−10 | 3.03E−04 | −0.366738389 | 0.366738389 | loss | 0.73529753 | 0.368559141 | LRCH1; LRCH1; LRCH1 | 37 | 13 |
| cg20421058 | 8.47E−10 | 3.60E−04 | −0.366562699 | 0.366562699 | loss | 0.758637287 | 0.392074558 | SORCS2 | 37 | 4 |
| cg18900591 | 7.13E−10 | 3.03E−04 | −0.366503003 | 0.366503003 | loss | 0.883333862 | 0.516830859 | SALL4; SALL4 | 37 | 20 |
| cg04413853 | 7.13E−10 | 3.03E−04 | −0.366491428 | 0.366491428 | loss | 0.729367328 | 0.3628759 | C17orf64 | 37 | 17 |
| cg12550344 | 2.06E−09 | 8.75E−04 | −0.366450163 | 0.366450163 | loss | 0.673770751 | 0.307320588 | | 37 | 5 |
| cg02331910 | 3.57E−09 | 1.52E−03 | −0.366420348 | 0.366420348 | loss | 0.581625123 | 0.215194775 | S100A13; S100A13; S100A13; | 37 | 1 |
| | | | | | | | | S100A13; S100A13; S100A13; | | |
| | | | | | | | | S100A1 | | |
| cg25716013 | 1.83E−09 | 7.75E−04 | −0.366389571 | 0.366389571 | loss | 0.6539733 | 0.287583729 | COX7A2; COX7A2 | 37 | 6 |
| cg20275928 | 7.13E−10 | 3.03E−04 | −0.366354896 | 0.366354896 | loss | 0.817663449 | 0.451308553 | LINC00482 | 37 | 17 |
| cg09366730 | 9.23E−10 | 3.92E−04 | −0.366351367 | 0.366351367 | loss | 0.810770079 | 0.444418712 | ZFYVE28; ZFYVE28; ZFYVE28; | 37 | 4 |
| | | | | | | | | ZFYVE28; ZFYVE28; ZFYVE28 | | |
| cg27532171 | 7.13E−10 | 3.03E−04 | −0.366290254 | 0.366290254 | loss | 0.74988313 | 0.383592876 | | 37 | 1 |
| cg06943627 | 1.10E−09 | 4.65E−04 | −0.365927217 | 0.365927217 | loss | 0.824664811 | 0.458737594 | NGFR; LOC100288866 | 37 | 17 |
| cg21191514 | 7.84E−10 | 3.33E−04 | −0.365631294 | 0.365631294 | loss | 0.52007835 | 0.154447056 | TH; TH; TH | 37 | 11 |
| cg21363811 | 1.02E−09 | 4.33E−04 | −0.365445032 | 0.365445032 | loss | 0.751959262 | 0.386514229 | FAM163A | 37 | 1 |
| cg00039463 | 7.13E−10 | 3.03E−04 | −0.365311417 | 0.365311417 | loss | 0.748919306 | 0.383607888 | CREBBP; CREBBP | 37 | 16 |
| cg12634306 | 5.39E−09 | 2.29E−03 | −0.365263719 | 0.365263729 | loss | 0.576987542 | 0.211723823 | HEYL | 37 | 1 |
| cg12865939 | 1.83E−09 | 7.75E−04 | −0.365262289 | 0.365262289 | loss | 0.785704672 | 0.420442382 | ST5; ST5; ST5; ST5 | 37 | 11 |
| cg14425869 | 7.13E−10 | 3.03E−04 | −0.365226179 | 0.365226179 | loss | 0.810641785 | 0.445415606 | | 37 | 9 |
| cg22902089 | 7.13E−10 | 3.03E−04 | −0.365122961 | 0.365122961 | loss | 0.700051849 | 0.334928888 | CREB3L1 | 37 | 11 |
| cg19153828 | 7.13E−10 | 3.03E−04 | −0.365094575 | 0.365094575 | loss | 0.547327091 | 0.182232515 | | 37 | 2 |
| cg01205935 | 7.13E−10 | 3.03E−04 | −0.36508261 | 0.36508261 | loss | 0.756577892 | 0.391495282 | DOCK9; DOCK9; DOCK9- | 37 | 13 |
| | | | | | | | | AS2 | | |
| cg09100338 | 7.13E−10 | 3.03E−04 | −0.365065906 | 0.365065906 | loss | 0.518420913 | 0.153355007 | | 37 | 8 |
| cg15015539 | 7.13E−10 | 3.03E−04 | −0.365041759 | 0.365041759 | loss | 0.518838891 | 0.153797131 | DES | 37 | 2 |
| cg17017404 | 7.13E−10 | 3.03E−04 | −0.36490429 | 0.36490429 | loss | 0.856937943 | 0.492033653 | ALX4 | 37 | 11 |
| cg00802903 | 9.23E−10 | 3.92E−04 | −0.3648913 | 0.3648913 | loss | 0.700482847 | 0.335591547 | | 37 | 7 |
| cg23006204 | 7.13E−10 | 3.03E−04 | −0.364810965 | 0.364810965 | loss | 0.673922794 | 0.309111829 | DPM2; DPM2 | 37 | 9 |
| cg08801479 | 7.13E−10 | 3.03E−04 | −0.364809496 | 0.364809496 | loss | 0.88879556 | 0.523986065 | NOTCH4 | 37 | 6 |
| cg23414861 | 1.10E−09 | 4.65E−04 | −0.364778866 | 0.364778866 | loss | 0.734586377 | 0.369807512 | DPPA5; DPPA5 | 37 | 6 |
| cg19286437 | 7.13E−10 | 3.03E−04 | −0.364726239 | 0.364726239 | loss | 0.842713957 | 0.477987718 | LDB3; LDB3; LDB3; LDB3; LDB3; | 37 | 10 |
| | | | | | | | | LDB3 | | |
| cg02085507 | 7.13E−10 | 3.03E−04 | −0.364666201 | 0.364666201 | loss | 0.555593072 | 0.190926871 | TRIP10 | 37 | 19 |
| cg23290482 | 8.47E−10 | 3.60E−04 | −0.364660077 | 0.364660077 | loss | 0.779527683 | 0.414867606 | C17orf98; C17orf98 | 37 | 17 |
| cg23533513 | 7.13E−10 | 3.03E−04 | −0.364637754 | 0.364637754 | loss | 0.454664358 | 0.090026604 | MYO7A; MYO7A; MYO7A | 37 | 11 |
| cg25731401 | 3.88E−09 | 1.65E−03 | −0.364587093 | 0.364587093 | loss | 0.54935073 | 0.184763638 | | 37 | 2 |
| cg13753527 | 7.13E−10 | 3.03E−04 | −0.364477751 | 0.364477751 | loss | 0.759794704 | 0.395316953 | | 37 | 9 |
| cg04406111 | 1.19E−09 | 5.07E−04 | −0.364384629 | 0.364384629 | loss | 0.766352064 | 0.401967435 | TACC2; TACC2 | 37 | 10 |
| cg01713950 | 7.13E−10 | 3.03E−04 | −0.364347643 | 0.364347643 | loss | 0.794870613 | 0.430522971 | RCC2 | 37 | 1 |
| cg12443001 | 7.13E−10 | 3.03E−04 | −0.364315966 | 0.364315966 | loss | 0.665447272 | 0.301131306 | | 37 | 1 |
| cg12516097 | 7.13E−10 | 3.03E−04 | −0.364005956 | 0.364005956 | loss | 0.919542174 | 0.555536218 | | 37 | 1 |
| cg08893853 | 7.13E−10 | 3.03E−04 | −0.363962635 | 0.363962635 | loss | 0.758701753 | 0.394739118 | RIIAD1 | 37 | 1 |
| cg25567448 | 7.13E−10 | 3.03E−04 | −0.363780948 | 0.363780948 | loss | 0.837386636 | 0.473605688 | NEURL3 | 37 | 2 |
| cg05118960 | 7.13E−10 | 3.03E−04 | −0.363747002 | 0.353747002 | loss | 0.828188496 | 0.464441494 | HYAL2; HYAL2 | 37 | 3 |
| cg08991643 | 7.13E−10 | 3.03E−04 | −0.363576816 | 0.363576816 | loss | 0.709191804 | 0.345614988 | | 37 | 3 |
| cg20597877 | 1.10E−09 | 4.65E−04 | −0.36355348 | 0.36355348 | loss | 0.731955751 | 0.368402271 | | 37 | 13 |
| cg09527615 | 1.73E−09 | 7.34E−04 | −0.363521053 | 0.363521053 | loss | 0.672878528 | 0.309357475 | | 37 | 11 |
| cg25439883 | 7.13E−10 | 3.03E−04 | −0.363504024 | 0.363504024 | loss | 0.72538703 | 0.361883006 | | 37 | 6 |
| cg07085632 | 7.13E−10 | 3.03E−04 | −0.36346981 | 0.36346981 | loss | 0.65588164 | 0.292411829 | | 37 | 11 |
| cg25317157 | 7.13E−10 | 3.03E−04 | −0.363274641 | 0.363274641 | loss | 0.546268811 | 0.182994171 | C10orf90 | 37 | 10 |
| cg05124918 | 7.13E−10 | 3.03E−04 | −0.363164952 | 0.363164952 | loss | 0.94064257 | 0.577477618 | | 37 | 12 |
| cg22175814 | 7.13E−10 | 3.03E−04 | −0.362836564 | 0.362836564 | loss | 0.743998623 | 0.381162059 | LOC100134391 | 37 | 17 |
| cg19721065 | 7.13E−10 | 3.03E−04 | −0.362820586 | 0.362820586 | loss | 0.774630509 | 0.411809924 | RASAL1; RASAL1; RASAL1 | 37 | 12 |
| cg17851725 | 7.13E−10 | 3.03E−04 | −0.362805288 | 0.362805288 | loss | 0.805912353 | 0.443107065 | | 37 | 2 |
| cg25607216 | 1.99E−09 | 8.43E−04 | −0.362741563 | 0.362741563 | loss | 0.785935587 | 0.423194024 | | 37 | 11 |
| cg14618634 | 7.13E−10 | 3.03E−04 | −0.362643585 | 0.362643585 | loss | 0.701419426 | 0.338775841 | | 37 | 9 |
| cg13793718 | 7.77E−10 | 3.30E−04 | −0.36236995 | 0.36236995 | loss | 0.740881479 | 0.378511529 | | 37 | 13 |
| cg05324407 | 7.13E−10 | 3.03E−04 | −0.362357046 | 0.362357046 | loss | 0.686518675 | 0.324161629 | | 37 | 16 |
| cg15247509 | 9.23E−10 | 3.92E−04 | −0.362253932 | 0.362253932 | loss | 0.856014774 | 0.493760841 | GDF2; GDF2 | 37 | 10 |
| cg07140497 | 7.13E−10 | 3.03E−04 | −0.36220345 | 0.36220345 | loss | 0.825140338 | 0.462936888 | RASAL1; RASAL1; RASAL1 | 37 | 12 |
| cg16208084 | 7.13E−10 | 3.03E−04 | −0.362197988 | 0.362197988 | loss | 0.9475441 | 0.585346112 | KAT2A; HSPB9 | 37 | 17 |
| cg24129222 | 7.13E−10 | 3.03E−04 | −0.362072195 | 0.362072195 | loss | 0.530160636 | 0.268088441 | C2orf81 | 37 | 2 |
| cg19829856 | 7.13E−10 | 3.03E−04 | −0.362028971 | 0.362028971 | loss | 0.980790742 | 0.618761771 | LOXHD1 | 37 | 18 |
| cg11512608 | 7.13E−10 | 3.03E−04 | −0.361994665 | 0.361994665 | loss | 0.8754495 | 0.513454835 | PLEKHB1; PLEKHB1; PLEKHB1; | 37 | 11 |
| | | | | | | | | PLEKHB1 | | |
| cg14269301 | 7.13E−10 | 3.03E−04 | −0.361868428 | 0.361868428 | loss | 0.701246598 | 0.33937817 | MAP3K15 | 37 | X |
| cg21923770 | 7.13E−10 | 3.03E−04 | −0.361696691 | 0.361696691 | loss | 0.835293438 | 0.473596747 | TUSC5 | 37 | 17 |
| cg19815818 | 7.13E−10 | 3.03E−04 | −0.361488665 | 0.361488665 | loss | 0.835718694 | 0.474230029 | | 37 | 17 |
| cg08404221 | 7.13E−10 | 3.03E−04 | −0.361468913 | 0.361468913 | loss | 0.427666104 | 0.066197191 | FBXO27; FBXO27 | 37 | 19 |
| cg23678210 | 8.47E−10 | 3.60E−04 | −0.361437924 | 0.361437924 | loss | 0.611903083 | 0.250465159 | PXT1 | 37 | 6 |
| cg23951474 | 7.13E−10 | 3.03E−04 | −0.361345105 | 0.361345105 | loss | 0.756234811 | 0.394889706 | TH; TH; TH | 37 | 11 |
| cg19679397 | 7.13E−10 | 3.03E−04 | −0.361318385 | 0.361318385 | loss | 0.821938691 | 0.460620306 | FADD | 37 | 11 |
| cg01568228 | 7.13E−10 | 3.03E−04 | −0.361229364 | 0.361229364 | loss | 0.728083628 | 0.366854265 | | 37 | 9 |
| cg13198297 | 7.13E−10 | 3.03E−04 | −0.3612103 | 0.3512103 | loss | 0.808172311 | 0.446962012 | MYO15A | 37 | 17 |
| cg21771200 | 7.77E−10 | 3.30E−04 | 0.361172983 | 0.361172983 | gain | 0.097806006 | 0.458978988 | ZNF833P | 37 | 19 |
| cg06230839 | 7.13E−10 | 3.03E−04 | −0.36115173 | 0.36115173 | loss | 0.655108583 | 0.293956853 | | 37 | 21 |
| cg02110835 | 7.13E−10 | 3.03E−04 | −0.361124358 | 0.361124358 | loss | 0.817423775 | 0.456299418 | | 37 | 17 |
| cg18750087 | 7.13E−10 | 3.03E−04 | 0.361106204 | 0.361106204 | loss | 0.658860198 | 0.297753994 | | 37 | 10 |
| cg23236370 | 7.13E−10 | 3.03E−04 | −0.36094752 | 0.36094752 | loss | 0.778433843 | 0.417486324 | ADAMTS12 | 37 | 5 |
| cg26145670 | 7.13E−10 | 3.03E−04 | −0.360876693 | 0.360876693 | loss | 0.67218514 | 0.311308447 | SCAND3 | 37 | 6 |
| cg14560110 | 7.13E−10 | 3.03E−04 | −0.360831864 | 0.360831864 | loss | 0.66582536 | 0.304993496 | CCDC144NL; CCDC144NL; | 37 | 17 |
| | | | | | | | | LOC440416 | | |
| cg11345143 | 7.13E−10 | 3.03E−04 | −0.360822958 | 0.360822958 | loss | 0.747653711 | 0.386840753 | CRYL1 | 37 | 13 |
| cg14614094 | 7.13E−10 | 3.03E−04 | −0.360726565 | 0.360726565 | loss | 0.733017089 | 0.372290524 | EXOSC2; EXOSC2; EXOSC2; | 37 | 9 |
| | | | | | | | | EXOSC2 | | |
| cg08804111 | 6.35E−09 | 2.70E−03 | −0.360368012 | 0.360368012 | loss | 0.701177947 | 0.340809935 | | 37 | 11 |
| cg19265480 | 7.84E−10 | 3.33E−04 | −0.360356594 | 0.360356594 | loss | 0.722952994 | 0.3625964 | NBPF8; NBPF8; NBPF8 | 37 | 1 |
| cg03292743 | 7.13E−10 | 3.03E−04 | −0.36026448 | 0.36026448 | loss | 0.594413709 | 0.234149229 | C2orf81 | 37 | 2 |
| cg09266924 | 7.77E−10 | 3.30E−04 | −0.360196044 | 0.360196044 | loss | 0.599398179 | 0.239202135 | LOC646278 | 37 | 15 |
| cg20066226 | 7.77E−10 | 3.30E−04 | −0.360073985 | 0.360073985 | loss | 0.695345585 | 0.3352716 | | 37 | 13 |
| cg05931423 | 1.57E−09 | 6.68E−04 | −0.36006224 | 0.36006224 | loss | 0.917528258 | 0.557466018 | | 37 | 5 |
| cg08435674 | 7.13E−10 | 3.03E−04 | −0.359763095 | 0.359763095 | loss | 0.620530183 | 0.260867088 | | 37 | 6 |
| cg04117972 | 9.23E−10 | 3.92E−04 | −0.359725177 | 0.359725177 | loss | 0.703960977 | 0.3442358 | | 37 | 1 |
| cg06347454 | 1.19E−09 | 5.07E−04 | −0.359705194 | 0.359705194 | loss | 0.882248458 | 0.522543265 | ABCA13 | 37 | 7 |
| cg26242687 | 7.77E−10 | 3.30E−04 | −0.359570464 | 0.359570464 | loss | 0.613665823 | 0.254095359 | | 37 | 6 |
| cg14654363 | 7.13E−10 | 3.03E−04 | −0.359499631 | 0.359499631 | loss | 0.740599902 | 0.381100271 | | 37 | 6 |
| cg05332854 | 7.13E−10 | 3.03E−04 | −0.359335809 | 0.359335809 | loss | 0.629611126 | 0.270275318 | | 37 | 16 |
| cg12754571 | 1.87E−09 | 7.93E−04 | −0.359263245 | 0.359263245 | loss | 0.60709406 | 0.247830815 | GCSAML; GCSAML; GCSAML; | 37 | 1 |
| | | | | | | | | GCSAML; GCSAML; GCSAM1; | | |
| | | | | | | | | OR2C3; GCSAML- | | |
| | | | | | | | | AS1; GCSAML-AS1 | | |
| cg00380835 | 7.84E−10 | 3.33E−04 | −0.359262062 | 0.359262062 | loss | 0.744947656 | 0.385685594 | SHANK1 | 37 | 19 |
| cg17482114 | 7.77E−10 | 3.30E−04 | −0.359205386 | 0.359205386 | loss | 0.835935604 | 0.476730218 | ADCY5; ADCY5 | 37 | 3 |
| cg03309252 | 7.77E−10 | 3.30E−04 | −0.359137887 | 0.359137887 | loss | 0.761784228 | 0.402646341 | | 37 | 9 |
| cg17279652 | 7.13E−10 | 3.03E−04 | −0.359070426 | 0.359070426 | loss | 0.968263879 | 0.609193453 | TRIM7; TRIM7; TRIM7; TRIM7; | 37 | 5 |
| | | | | | | | | TRIM7 | | |
| cg26715042 | 1.42E−09 | 6.01E−04 | −0.358921903 | 0.358921903 | loss | 0.649149885 | 0.290227982 | CRISP2; CRISP2; CRISP2; CRISP2; | 37 | 6 |
| | | | | | | | | CRISP2; CRISP2; CRISP2; | | |
| | | | | | | | | CRISP2; CR1SP2; CR1SP2; | | |
| | | | | | | | | CRISP2; CRISP2 | | |
| cg08895590 | 1.43E−08 | 6.06E−03 | −0.358908604 | 0.358908604 | loss | 0.566550257 | 0.207641653 | | 37 | 1 |
| cg20011248 | 1.73E−09 | 7.34E−04 | −0.358893807 | 0.358893807 | loss | 0.516927596 | 0.158033789 | | 37 | 1 |
| cg11911679 | 5.84E−08 | 2.48E−02 | −0.358798317 | 0.358798317 | loss | 0.830666375 | 0.471868059 | CERKL; CERKL; CERKL; CERKL; | 37 | 2 |
| | | | | | | | | CERKL; CERKL; CERKL | | |
| cg21436055 | 7.13E−10 | 3.03E−04 | −0.358596207 | 0.358596207 | loss | 0.728040013 | 0.369443806 | | 37 | 1 |
| cg07225641 | 7.13E−10 | 3.03E−04 | −0.358558982 | 0.358558982 | loss | 0.542459758 | 0.183900776 | TDGF1; TDGF1 | 37 | 3 |
| cg05927817 | 1.10E−09 | 4.65E−04 | −0.35849651 | 0.35849651 | loss | 0.792759145 | 0.434262635 | | 37 | 6 |
| cg12243133 | 7.13E−10 | 3.03E−04 | −0.358494867 | 0.358494867 | loss | 0.651441402 | 0.292946535 | RHOD | 37 | 11 |
| cg08592890 | 7.13E−10 | 3.03E−04 | −0.358491028 | 0.358491028 | loss | 0.854311545 | 0.495820518 | CTF1; CTF1 | 37 | 16 |
| cg03215599 | 7.13E−10 | 3.03E−04 | −0.35846959 | 0.35846959 | loss | 0.891241408 | 0.532771818 | | 37 | 6 |
| cg21198712 | 7.13E−10 | 3.03E−04 | −0.358325638 | 0.358325638 | loss | 0.700427679 | 0.342102041 | | 37 | 9 |
| cg11644585 | 7.13E−10 | 3.03E−04 | −0.358292699 | 0.358292699 | loss | 0.96324707 | 0.604954371 | NTN1 | 37 | 17 |
| cg22878388 | 9.23E−10 | 3.92E−04 | −0.358120319 | 0.358120319 | loss | 0.632343977 | 0.274223659 | | 37 | 2 |
| cg26645709 | 1.73E−09 | 7.34E−04 | −0.358056252 | 0.358056252 | loss | 0.562059083 | 0.204002831 | | 37 | 15 |
| cg24942922 | 7.77E−10 | 3.30E−04 | −0.358040861 | 0.358040861 | loss | 0.718389985 | 0.360349124 | | 37 | 3 |
| cg05934682 | 7.13E−10 | 3.03E−04 | −0.357983459 | 0.357983459 | loss | 0.908292742 | 0.550309282 | IL4I1; IL4I1; IL4I1; IL4I1; IL4I1 | 37 | 19 |
| cg20457732 | 7.13E−10 | 3.03E−04 | −0.35773345 | 0.35773345 | loss | 0.419608885 | 0.061875435 | | 37 | 1 |
| cg15587476 | 7.13E−10 | 3.03E−04 | −0.357688107 | 0.357688107 | loss | 0.476514874 | 0.118826767 | FAM228A | 37 | 2 |
| cg10551329 | 7.13E−10 | 3.03E−04 | −0.357617398 | 0.357617398 | loss | 0.875334904 | 0.517717506 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg20736031 | 1.68E−09 | 7.12E−04 | −0.357600306 | 0.357600306 | loss | 0.753910906 | 0.3963106 | | 37 | 12 |
| cg17465304 | 7.13E−10 | 3.03E−04 | −0.357587743 | 0.357587743 | loss | 0.734518008 | 0.376930265 | KIF12; KIF12 | 37 | 9 |
| cg04357965 | 7.13E−10 | 3.03E−04 | −0.357555719 | 0.357555719 | loss | 0.761738566 | 0.404182847 | LHFPL2 | 37 | 5 |
| cg13110574 | 7.13E−10 | 3.03E−04 | −0.357547698 | 0.357547698 | loss | 0.815699257 | 0.458151559 | ADARB1; ADARB1; ADARB1; | 37 | 21 |
| | | | | | | | | ADARB1; SSR4P1; SSR4P1; | | |
| | | | | | | | | ADARB1; ADARB1; ADARB1; | | |
| | | | | | | | | ADARB1 | | |
| cg22302929 | 7.13E−10 | 3.03E−04 | −0.357330851 | 0.357330851 | loss | 0.801410045 | 0.444079194 | SCAND3 | 37 | 6 |
| cg18184769 | 7.13E−10 | 3.03E−04 | −0.357186356 | 0.357186356 | loss | 0.733793779 | 0.376607424 | MFN2; MFN2 | 37 | 1 |
| cg23869158 | 7.13E−10 | 3.03E−04 | −0.357125948 | 0.357125948 | loss | 0.742811577 | 0.385685629 | LRCH1; LRCH1; LRCH1 | 37 | 13 |
| cg16155393 | 7.13E−10 | 3.03E−04 | −0.357043959 | 0.357043959 | loss | 0.941228842 | 0.584184882 | TDGP1 | 37 | 3 |
| cg17277199 | 1.73E−09 | 7.34E−04 | −0.357030337 | 0.357030337 | loss | 0.460305855 | 0.103275518 | FAM228A | 37 | 2 |
| cg01194320 | 1.01E−09 | 4.27E−04 | −0.356928668 | 0.356928668 | loss | 0.730433621 | 0.373504953 | | 37 | 16 |
| cg25138412 | 1.83E−09 | 7.75E−04 | −0.356798987 | 0.356798987 | loss | 0.767477292 | 0.410678306 | | 37 | 1 |
| cg06259612 | 7.13E−10 | 3.03E−04 | −0.356720285 | 0.356720285 | loss | 0.926928079 | 0.570207794 | | 37 | 12 |
| cg07751331 | 7.13E−10 | 3.03E−04 | −0.356460926 | 0.356460926 | loss | 0.812905426 | 0.4564445 | KIF3C | 37 | 2 |
| cg26175343 | 7.13E−10 | 3.03E−04 | −0.356459051 | 0.356459051 | loss | 0.854382187 | 0.497923135 | LRRN4; LRRN4 | 37 | 20 |
| cg04981619 | 7.13E−10 | 3.03E−04 | −0.356266619 | 0.356266619 | loss | 0.572682225 | 0.216415606 | | 37 | 2 |
| cg10755878 | 1.99E−09 | 8.43E−04 | −0.356223937 | 0.356223937 | loss | 0.598870008 | 0.242646071 | | 37 | 2 |
| cg12934258 | 3.02E−09 | 1.28E−03 | −0.356217002 | 0.356217002 | loss | 0.690573349 | 0.334356347 | SYTL3; SYTL3; SYTL3; SYTL3 | 37 | 6 |
| cg14565151 | 7.77E−10 | 3.30E−04 | −0.356215887 | 0.356215887 | loss | 0.58615077 | 0.229934882 | USP44; USP44 | 37 | 12 |
| cg03359095 | 7.13E−10 | 3.03E−04 | −0.355907076 | 0.355907076 | loss | 0.613396211 | 0.257489135 | RBBP8NL | 37 | 20 |
| cg25937577 | 7.13E−10 | 3.03E−04 | −0.355880312 | 0.355880312 | loss | 0.586741383 | 0.230861071 | PLA2G3; PLA2G3 | 37 | 22 |
| cg09243648 | 7.13E−10 | 3.03E−04 | −0.355835125 | 0.355835125 | loss | 0.940137108 | 0.584301982 | | 37 | 17 |
| cg06210630 | 1.30E−09 | 5.52E−04 | −0.355787339 | 0.355787339 | loss | 0.791964757 | 0.436177418 | ATP1A2 | 37 | 1 |
| cg09547692 | 4.57E−09 | 1.94E−03 | −0.355753681 | 0.355753681 | loss | 0.81646894 | 0.460715259 | | 37 | 1 |
| cg25520960 | 7.13E−10 | 3.03E−04 | −0.355478366 | 0.355478366 | loss | 0.781066155 | 0.425587788 | | 37 | 2 |
| cg20871559 | 7.84E−10 | 3.33E−04 | −0.355408146 | 0.355408146 | loss | 0.820287969 | 0.464879824 | | 37 | 1 |
| cg19739906 | 9.23E−10 | 3.92E−04 | −0.355347593 | 0.355347593 | loss | 0.623271875 | 0.267924282 | HNMT; HNMT; HNMT | 37 | 2 |
| cg05618807 | 7.13E−10 | 3.03E−04 | −0.355215607 | 0.355215607 | loss | 0.664298372 | 0.309082765 | | 37 | 19 |
| cg09669754 | 9.23E−10 | 3.92E−04 | −0.355131853 | 0.355131853 | loss | 0.768612153 | 0.4134803 | CARD14; CARD14; CARD14; | 37 | 17 |
| | | | | | | | | CARD14 | | |
| cg04177826 | 7.13E−10 | 3.03E−04 | −0.355085493 | 0.355035493 | loss | 0.522769966 | 0.167684473 | KLHDC7B | 37 | 22 |
| cg13064789 | 7.13E−10 | 3.03E−04 | −0.35506182 | 0.35506182 | loss | 0.597972491 | 0.242910671 | | 37 | 1 |
| cg08234664 | 1.73E−09 | 7.34E−04 | −0.355049113 | 0.355049113 | loss | 0.502515902 | 0.147465789 | LAMB2; LAMB2 | 37 | 3 |
| cg00349061 | 7.13E−10 | 3.03E−04 | 0.354991317 | 0.354991317 | loss | 0.576192906 | 0.221201588 | SLC30A2; SLC30A2 | 37 | 1 |
| cg23694533 | 7.13E−10 | 3.03E−04 | −0.354967755 | 0.354967755 | loss | 0.646760138 | 0.291792382 | | 37 | 17 |
| cg08571918 | 1.99E−09 | 8.43E−04 | −0.354894845 | 0.354894845 | loss | 0.595193862 | 0.240299018 | | 37 | 2 |
| cg08133631 | 8.11E−09 | 3.44E−03 | −0.354875988 | 0.354875988 | loss | 0.494799891 | 0.139923903 | CATSPER4 | 37 | 1 |
| cg21929564 | 7.13E−10 | 3.03E−04 | −0.354875796 | 0.354875796 | loss | 0.470191566 | 0.11531577 | CCDC152 | 37 | 5 |
| cg16913064 | 7.13E−10 | 3.03E−04 | −0.354759666 | 0.354759666 | loss | 0.465956796 | 0.111197131 | | 37 | 17 |
| cg06829154 | 7.13E−10 | 3.03E−04 | −0.354646112 | 0.354646112 | loss | 0.591583206 | 0.336937094 | | 37 | 2 |
| cg11704490 | 7.13E−10 | 3.03E−04 | −0.354635228 | 0.354635228 | loss | 0.720330028 | 0.3656948 | | 37 | 2 |
| cg26337070 | 1.83E−09 | 7.75E−04 | −0.354498307 | 0.354498307 | loss | 0.836010666 | 0.481512359 | ATOH8 | 37 | 2 |
| cg27111444 | 7.13E−10 | 3.03E−04 | −0.354494166 | 0.354494166 | loss | 0.741982319 | 0.387488153 | | 37 | 12 |
| cg15078284 | 7.13E−10 | 3.03E−04 | −0.354420245 | 0.354420245 | loss | 0.42128844 | 0.066868195 | DMKN; DMKN; DMKN; DMKN; | 37 | 19 |
| | | | | | | | | DMKN; DMKN; DMKN | | |
| cg05560575 | 7.13E−10 | 3.03E−04 | −0.354338988 | 0.354338988 | loss | 0.932001894 | 0.577662906 | | 37 | 12 |
| cg12193833 | 7.13E−10 | 3.03E−04 | −0.354235972 | 0.354235972 | loss | 0.39559653 | 0.041360658 | | 37 | 17 |
| cg07506749 | 7.13E−10 | 3.03E−04 | −0.354225502 | 0.354225502 | loss | 0.77853246 | 0.424306959 | | 37 | 16 |
| cg25866552 | 7.13E−10 | 3.03E−04 | −0.35420576 | 0.35420576 | loss | 0.512371142 | 0.158165381 | | 37 | 7 |
| cg26907313 | 7.13E−10 | 3.03E−04 | −0.354205711 | 0.354205711 | loss | 0.86299377 | 0.508788059 | ATP6V1B1 | 37 | 2 |
| cg23378165 | 7.13E−10 | 3.03E−04 | −0.354149928 | 0.354149928 | loss | 0.597654528 | 0.2435046 | USP43; USP43 | 37 | 17 |
| cg21852429 | 7.13E−10 | 3.03E−04 | −0.353864019 | 0.353864019 | loss | 0.847868708 | 0.494004688 | C17orf98; C17orf98 | 37 | 17 |
| cg09374627 | 9.23E−10 | 3.92E−04 | −0.353764354 | 0.353764354 | loss | 0.811792419 | 0.458028065 | | 37 | 14 |
| cg17149103 | 7.13E−10 | 3.03E−04 | −0.353692955 | 0.353692965 | loss | 0.7808034 | 0.427110435 | NBPF8; NBPF8; N9PF8 | 37 | 1 |
| cg18098722 | 7.13E−10 | 3.03E−04 | −0.353571312 | 0.353571312 | loss | 0.852231289 | 0.498659976 | C11orf35 | 37 | 11 |
| cg09264065 | 7.13E−10 | 3.03E−04 | −0.353362935 | 0.353362935 | loss | 0.530051072 | 0.176698136 | | 37 | 1 |
| cg10563109 | 2.56E−09 | 1.09E−03 | 0.353347958 | 0.353347958 | loss | 0.648215698 | 0.294867741 | | 37 | 6 |
| cg02698580 | 1.10E−09 | 4.65E−04 | −0.353345552 | 0.353345552 | loss | 0.742830523 | 0.389484971 | RGR; RGR; RGR | 37 | 10 |
| cg10775231 | 7.13E−10 | 3.03E−04 | −0.353341926 | 0.353341926 | loss | 0.772619696 | 0.419277771 | LINC00857 | 37 | 10 |
| cg26173906 | 7.84E−10 | 3.33E−04 | −0.353333099 | 0.353333099 | loss | 0.698580563 | 0.345247465 | | 37 | 19 |
| cg07524919 | 1.11E−09 | 4.72E−04 | −0.353228838 | 0.353228838 | loss | 0.54574049 | 0.192511652 | TNXB | 37 | 6 |
| cg12388541 | 7.13E−10 | 3.03E−04 | −0.353184372 | 0.353184372 | loss | 0.850295466 | 0.497111094 | | 37 | 6 |
| cg08457421 | 7.13E−10 | 3.03E−04 | −0.353139257 | 0.353139257 | loss | 0.886072087 | 0.532932829 | PLA2G3; PLA2G3 | 37 | 22 |
| cg24727290 | 7.13E−10 | 3.03E−04 | −0.353125373 | 0.353125373 | loss | 0.797283491 | 0.444158118 | DDR1; DDR1 | 37 | 6 |
| cg01517680 | 2.22E−09 | 9.41E−04 | −0.352999091 | 0.352999091 | loss | 0.46455852 | 0.111566761 | | 37 | 16 |
| cg02399371 | 7.13E−10 | 3.03E−04 | −0.352970782 | 0.352970782 | loss | 0.53374397 | 0.180773188 | | 37 | 17 |
| cg00216693 | 8.56E−10 | 3.63E−04 | −0.352892007 | 0.352892007 | loss | 0.945873925 | 0.592981918 | | 37 | 2 |
| cg20328456 | 7.13E−10 | 3.03E−04 | −0.35285882 | 0.35285882 | loss | 0.678881532 | 0.326022712 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg01297627 | 7.13E−10 | 3.03E−04 | −0.352822704 | 0.352822704 | loss | 0.785476809 | 0.432654106 | FAM71E2; FAM71E2 | 37 | 19 |
| cg26827653 | 7.13E−10 | 3.03E−04 | −0.352781047 | 0.352781047 | loss | 0.911569842 | 0.558788794 | FEZ1; FEZ1 | 37 | 11 |
| cg12150256 | 7.13E−10 | 3.03E−04 | −0.352703705 | 0.352703705 | loss | 0.789663781 | 0.436960076 | HYAL2; HYAL2 | 37 | 3 |
| cg21187770 | 7.13E−10 | 3.03E−04 | −0.352595108 | 0.352595108 | loss | 0.864856049 | 0.512260941 | KIF3C | 37 | 2 |
| cg11455444 | 7.13E−10 | 3.03E−04 | −0.352561319 | 0.352561319 | loss | 0.568048808 | 0.215487488 | LINC01101; LINC01101 | 37 | 2 |
| cg12560020 | 1.10E−09 | 4.65E−04 | −0.35250223 | 0.35250223 | loss | 0.509379349 | 0.156877119 | | 37 | 15 |
| cg07833467 | 7.13E−10 | 3.03E−04 | −0.352453904 | 0.352453904 | loss | 0.495392949 | 0.142939045 | KLHDC7B | 37 | 22 |
| cg07313064 | 7.13E−10 | 3.03E−04 | −0.352387724 | 0.352387724 | loss | 0.7561637 | 0.403775976 | | 37 | 7 |
| cg20498637 | 7.13E−10 | 3.03E−04 | −0.352386522 | 0.352386522 | loss | 0.734758698 | 0.382372176 | | 37 | 6 |
| cg00639615 | 7.13E−10 | 3.03E−04 | −0.352249027 | 0.352249027 | loss | 0.498973057 | 0.146724029 | FANCI; FANCI | 37 | 15 |
| cg22697684 | 7.13E−10 | 3.03E−04 | −0.352248015 | 0.352248015 | loss | 0.868999374 | 0.516751359 | USP43; USP43 | 37 | 17 |
| cg18364858 | 7.13E−10 | 3.03E−04 | −0.352174464 | 0.352174464 | loss | 0.526405913 | 0.174231449 | | 37 | 14 |
| cg25582488 | 7.13E−10 | 3.03E−04 | −0.352161755 | 0.352161755 | loss | 0.419598747 | 0.067436992 | | 37 | 14 |
| cg26207380 | 7.77E−10 | 3.30E−04 | −0.352102728 | 0.352102728 | loss | 0.606691528 | 0.2545888 | | 37 | 8 |
| cg13752184 | 7.13E−10 | 3.03E−04 | −0.35205993 | 0.35205993 | loss | 0.730749836 | 0.378689906 | | 37 | 13 |
| cg26817145 | 2.69E−08 | 1.14E−02 | −0.352052581 | 0.352052581 | loss | 0.75784764 | 0.405795059 | THSD4 | 37 | 15 |
| cg20559189 | 7.13E−10 | 3.03E−04 | −0.351984519 | 0.351984519 | loss | 0.808682972 | 0.456698453 | TUB; TUB | 37 | 11 |
| cg23009780 | 7.13E−10 | 3.03E−04 | −0.35195356 | 0.35195356 | loss | 0.678100719 | 0.326147159 | | 37 | 2 |
| cg08951638 | 7.13E−10 | 3.03E−04 | −0.351936801 | 0.351936801 | loss | 0.553471013 | 0.201534212 | | 37 | 5 |
| cg02992311 | 7.13E−10 | 3.03E−04 | −0.351622012 | 0.351622012 | loss | 0.709533906 | 0.357911894 | FBXO27 | 37 | 19 |
| cg11724147 | 7.13E−10 | 3.03E−04 | −0.351494922 | 0.351494922 | loss | 0.816493351 | 0.464998429 | HIBADH | 37 | 7 |
| cg01090821 | 7.13E−10 | 3.03E−04 | −0.351373499 | 0.351373499 | loss | 0.767586464 | 0.416212965 | | 37 | 2 |
| cg01987536 | 7.13E−10 | 3.03E−04 | −0.351253932 | 0.351253932 | loss | 0.661692543 | 0.310438612 | AK7 | 37 | 14 |
| cg26630791 | 1.22E−08 | 5.16E−03 | −0.351137611 | 0.351137611 | loss | 0.611341323 | 0.260203712 | HAPLN1; HAPLN1 | 37 | 5 |
| cg21205855 | 8.47E−10 | 3.60E−04 | −0.351129164 | 0.351129164 | loss | 0.655926023 | 0.304796859 | | 37 | 8 |
| cg08731696 | 7.13E−10 | 3.03E−04 | −0.350955171 | 0.350955171 | loss | 0.864509683 | 0.513554512 | LOC100131060; LOC100131060 | 37 | 1 |
| cg07945733 | 7.13E−10 | 3.03E−04 | −0.350934204 | 0.350934204 | loss | 0.573010057 | 0.222075853 | C10orf82; C10orf82 | 37 | 10 |
| cg25921357 | 7.13E−10 | 3.03E−04 | −0.35089963 | 0.35089963 | loss | 0.70914153 | 0.3582419 | TMEM190 | 37 | 19 |
| cg00320094 | 8.55E−08 | 3.63E−02 | −0.350885875 | 0.350885875 | loss | 0.686017245 | 0.335131371 | WLS; WLS; WLS | 37 | 1 |
| cg26873880 | 4.21E−09 | 1.79E−03 | −0.350830226 | 0.350830226 | loss | 0.740201332 | 0.389371106 | | 37 | 6 |
| cg12948059 | 7.13E−10 | 3.03E−04 | −0.350766786 | 0.350766786 | loss | 0.472479368 | 0.121712582 | | 37 | 2 |
| cg27009812 | 7.13E−10 | 3.03E−04 | −0.350751175 | 0.350751175 | loss | 0.470144721 | 0.119393545 | CLDN9 | 37 | 16 |
| cg14887505 | 7.13E−10 | 3.03E−04 | −0.350747885 | 0.350747885 | loss | 0.881769243 | 0.531021359 | | 37 | 7 |
| cg07223180 | 7.13E−10 | 3.03E−04 | −0.35064731 | 0.35064731 | loss | 0.571626287 | 0.220978976 | CRYL1 | 37 | 13 |
| cg08951271 | 7.13E−10 | 3.03E−04 | −0.350639539 | 0.350639539 | loss | 0.827359004 | 0.476719465 | DDR1 | 37 | 6 |
| cg23693749 | 3.02E−09 | 1.28E−03 | −0.35060529 | 0.35060529 | loss | 0.717673949 | 0.367068659 | | 37 | 18 |
| cg08436104 | 2.56E−09 | 1.09E−03 | −0.350582032 | 0.350582032 | loss | 0.688430938 | 0.337848906 | | 37 | 5 |
| cg04637704 | 7.13E−10 | 3.03E−04 | −0.35052165 | 0.35052165 | loss | 0.931387715 | 0.580866065 | TNPO3; TNPO3; TPI1P2; TNPO3 | 37 | 7 |
| cg13245228 | 7.13E−10 | 3.03E−04 | −0.350343785 | 0.350343785 | loss | 0.743493643 | 0.393149859 | | 37 | 8 |
| cg16083044 | 7.13E−10 | 3.03E−04 | −0.350307246 | 0.350307246 | loss | 0.886269075 | 0.535961829 | | 37 | 6 |
| cg21204522 | 9.54E−09 | 4.05E−03 | −0.350291788 | 0.350291788 | loss | 0.945598057 | 0.595306269 | LOC100131289 | 37 | 6 |
| cg02630477 | 7.13E−10 | 3.03E−04 | −0.350201403 | 0.350201403 | loss | 0.889490385 | 0.539288982 | | 37 | 1 |
| cg14904725 | 7.13E−10 | 3.03E−04 | −0.350062946 | 0.350062946 | loss | 0.671585234 | 0.321522288 | F10 | 37 | 13 |
| cg10537450 | 7.13E−10 | 3.03E−04 | −0.350060378 | 0.350060378 | loss | 0.459563945 | 0.109503568 | NBPF8; NBPF8; NBPF8 | 37 | 1 |
| cg15093383 | 7.13E−10 | 3.03E−04 | −0.349952571 | 0.349952571 | loss | 0.834637683 | 0.484685112 | | 37 | 17 |
| cg02566259 | 7.13E−10 | 3.03E−04 | −0.349951576 | 0.349951576 | loss | 0.444613258 | 0.094661682 | | 37 | 4 |
| cg16415457 | 7.13E−10 | 3.03E−04 | −0.349673204 | 0.349673204 | loss | 0.453914998 | 0.104241794 | | 37 | 3 |
| cg07083902 | 7.13E−10 | 3.03E−04 | −0.349485722 | 0.349485722 | loss | 0.687865192 | 0.338379471 | | 37 | 6 |
| cg00632007 | 7.13E−10 | 3.03E−04 | −0.349442731 | 0.349442731 | loss | 0.80531606 | 0.455873329 | | 37 | 6 |
| cg26321999 | 7.13E−10 | 3.03E−04 | −0.349415084 | 0.349415084 | loss | 0.512178215 | 0.162763131 | DDR1; DDR1; DDR1 | 37 | 6 |
| cg20449659 | 1.83E−09 | 7.75E−04 | −0.349283498 | 0.349283498 | loss | 0.908670957 | 0.559387459 | WT1; WT1; WT1; WT1; WT1 | 37 | 11 |
| cg24648385 | 7.13E−10 | 3.03E−04 | −0.34921456 | 0.34921456 | loss | 0.529009913 | 0.179795353 | | 37 | 2 |
| cg11124420 | 1.19E−09 | 5.07E−04 | −0.349177007 | 0.349177007 | loss | 0.899935919 | 0.550758912 | | 37 | 9 |
| cg21850518 | 7.13E−10 | 3.03E−04 | −0.34909075 | 0.34909075 | loss | 0.482412768 | 0.133322018 | | 37 | 17 |
| cg21658372 | 7.13E−10 | 3.03E−04 | −0.349078907 | 0.349078907 | loss | 0.898368413 | 0.549289506 | NTN5; SEC1P | 37 | 19 |
| cg20415053 | 9.54E−09 | 4.05E−03 | −0.348901736 | 0.348901736 | loss | 0.395004019 | 0.046102283 | CATSPER4 | 37 | 1 |
| cg16059991 | 2.16E−09 | 9.17E−04 | −0.348845845 | 0.348845845 | loss | 0.725101051 | 0.376255206 | | 37 | 15 |
| cg25037000 | 7.13E−10 | 3.03E−04 | −0.348780592 | 0.348780592 | loss | 0.786672262 | 0.437891671 | | 37 | 1 |
| cg16268165 | 7.13E−10 | 3.03E−04 | −0.348740857 | 0.348740857 | loss | 0.601549621 | 0.252808764 | UNC5A | 37 | 5 |
| cg10075506 | 6.35E−09 | 2.70E−03 | −0.348704147 | 0.348704147 | loss | 0.730135958 | 0.381431812 | MYT1L | 37 | 2 |
| cg00291837 | 2.48E−08 | 1.05E−02 | −0.348589207 | 0.348589207 | loss | 0.731501866 | 0.382912659 | | 37 | 1 |
| cg25122233 | 7.13E−10 | 3.03E−04 | −0.348577922 | 0.348577922 | loss | 0.573690034 | 0.225112112 | HOXC4; HOXC6; HOXC5 | 37 | 12 |
| cg00760562 | 7.77E−10 | 3.30E−04 | −0.348541989 | 0.348541989 | loss | 0.726200225 | 0.377658235 | SLC51A | 37 | 3 |
| cg24833819 | 7.77E−10 | 3.30E−04 | −0.348496661 | 0.348496661 | loss | 0.734248955 | 0.385752294 | | 37 | 5 |
| cg10517650 | 1.01E−09 | 4.27E−04 | −0.348486587 | 0.348486587 | loss | 0.46068583 | 0.112199243 | SPICE1 | 37 | 3 |
| cg25618731 | 7.77E−10 | 3.30E−04 | −0.348436362 | 0.348436362 | loss | 0.584692768 | 0.236256406 | PRSS41 | 37 | 16 |
| cg03662997 | 7.13E−10 | 3.03E−04 | −0.348301517 | 0.348301517 | loss | 0.757678958 | 0.409377441 | | 37 | 11 |
| cg00597076 | 7.13E−10 | 3.03E−04 | −0.348282319 | 0.348282319 | loss | 0.567989466 | 0.219707147 | MYO1C; MYO1C; MYO1C | 37 | 17 |
| cg26046136 | 1.44E−09 | 6.12E−04 | −0.348029776 | 0.348029776 | loss | 0.79248974 | 0.444459965 | EYA2; EYA2 | 37 | 20 |
| cg21687740 | 7.13E−10 | 3.03E−04 | −0.347966306 | 0.347966306 | loss | 0.440245662 | 0.092279356 | | 37 | 1 |
| cg02881684 | 1.42E−09 | 6.01E−04 | 0.347842221 | 0.347842221 | loss | 0.593725609 | 0.245883388 | FOSB; FOSB | 37 | 19 |
| cg11550547 | 7.13E−10 | 3.03E−04 | −0.34767054 | 0.34767054 | loss | 0.65797567 | 0.310305129 | NOUFC2- | 37 | 11 |
| | | | | | | | | KCTD14; NDUFC2- | | |
| | | | | | | | | KCTD14; NDUFC2- | | |
| | | | | | | | | KCTD14; KCTD14; KCTD14; | | |
| | | | | | | | | KCTD14 | | |
| cg16800939 | 7.13E−10 | 3.03E−04 | −0.347660262 | 0.347660262 | loss | 0.582496126 | 0.234835865 | | 37 | 14 |
| cg19484305 | 8.47E−10 | 3.60E−04 | −0.347547564 | 0.347547564 | loss | 0.66106877 | 0.313521206 | | 37 | 11 |
| cg22842797 | 7.84E−10 | 3.33E−04 | −0.347410462 | 0.347410462 | loss | 0.559285656 | 0.211875194 | WFIKKN2 | 37 | 17 |
| cg15526213 | 7.13E−10 | 3.03E−04 | −0.347409015 | 0.347409015 | loss | 0.816010626 | 0.468601612 | | 37 | 10 |
| cg03357999 | 7.13E−10 | 3.03E−04 | −0.347304937 | 0.347304937 | loss | 0.499651594 | 0.152346657 | | 37 | 7 |
| cg21025349 | 7.13E−10 | 3.03E−04 | −0.347244543 | 0.347244543 | loss | 0.742095408 | 0.394851865 | KIRREL3; KIRREL3 | 37 | 11 |
| cg04322572 | 7.13E−10 | 3.03E−04 | −0.347156674 | 0.347156674 | loss | 0.917151209 | 0.569994535 | | 37 | 10 |
| cg19510698 | 7.13E−10 | 3.03E−04 | −0.347108355 | 0.347108355 | loss | 0.710857643 | 0.363749288 | ALDH1A3 | 37 | 15 |
| cg02086195 | 7.13E−10 | 3.03E−04 | −0.347046507 | 0.347046507 | loss | 0.59218796 | 0.245141453 | DHRS3 | 37 | 1 |
| cg19907915 | 7.48E−09 | 3.18E−03 | −0.34701561 | 0.34701561 | loss | 0.680575045 | 0.333559435 | IGSF98 | 37 | 11 |
| cg11631063 | 7.13E−10 | 3.03E−04 | −0.346991893 | 0.346991893 | loss | 0.64625264 | 0.299260747 | | 37 | 4 |
| cg25035376 | 7.13E−10 | 3.03E−04 | −0.346918204 | 0.346918204 | loss | 0.84082794 | 0.493909735 | KIF5A | 37 | 12 |
| cg18348654 | 7.13E−10 | 3.03E−04 | −0.346875003 | 0.346875003 | loss | 0.714933962 | 0.368058959 | | 37 | 18 |
| cg23042086 | 7.84E−10 | 3.33E−04 | −0.346736163 | 0.346736163 | loss | 0.691636892 | 0.344900729 | | 37 | 8 |
| cg21217540 | 8.47E−10 | 3.60E−04 | −0.346665016 | 0.346665016 | loss | 0.748592198 | 0.401927182 | KCNC4; KCNC4; KCNC4 | 37 | 1 |
| cg19137107 | 7.13E−10 | 3.03E−04 | −0.346632717 | 0.346632717 | loss | 0.517053208 | 0.170420491 | | 37 | 11 |
| cg14379965 | 7.13E−10 | 3.03E−04 | −0.34655148 | 0.34655148 | loss | 0.575956568 | 0.229405088 | | 37 | 2 |
| cg24652615 | 3.57E−09 | 1.52E−03 | −0.346367706 | 0.346367706 | loss | 0.729198558 | 0.382830853 | TMEM1518 | 37 | 6 |
| cg02193146 | 8.47E−10 | 3.60E−04 | −0.346352652 | 0.346352652 | loss | 0.735922175 | 0.389569524 | KCNC4; KCNC4; KCNC4 | 37 | 1 |
| cg18861753 | 1.54E−08 | 6.56E−03 | −0.346309419 | 0.346309419 | loss | 0.773895266 | 0.427585847 | COL23A1 | 37 | 5 |
| cg24129115 | 1.99E−09 | 8.43E−04 | −0.346117642 | 0.346117642 | loss | 0.845299077 | 0.499181435 | REM2 | 37 | 14 |
| cg19526353 | 7.13E−10 | 3.03E−04 | −0.346109588 | 0.346109588 | loss | 0.886746553 | 0.540636965 | ERF | 37 | 19 |
| cg05115664 | 7.13E−10 | 3.03E−04 | −0.346100424 | 0.346100424 | loss | 0.78022773 | 0.434127306 | | 37 | 9 |
| cg18140268 | 7.13E−10 | 3.03E−04 | −0.34606861 | 0.34606861 | loss | 0.654274151 | 0.308205541 | NBPF8; NBPF8; NBPF8 | 37 | 1 |
| cg11206167 | 7.13E−10 | 3.03E−04 | −0.346044956 | 0.346044956 | loss | 0.793716662 | 0.447671706 | | 37 | 5 |
| cg05105069 | 7.13E−10 | 3.03E−04 | −0.345905553 | 0.345905553 | loss | 0.572336089 | 0.226430535 | TCEAL7 | 37 | X |
| cg26331343 | 7.84E−10 | 3.33E−04 | −0.345857439 | 0.345857439 | loss | 0.717284104 | 0.371425665 | LINC00880; LINC00881 | 37 | 3 |
| cg05865340 | 7.13E−10 | 3.03E−04 | −0.345850916 | 0.345850916 | loss | 0.486010508 | 0.140159592 | | 37 | 2 |
| cg15127250 | 7.13E−10 | 3.03E−04 | −0.345781221 | 0.345781221 | loss | 0.496757349 | 0.150975128 | SLC4A8; SLC4A8; GALNT6 | 37 | 12 |
| cg19731612 | 7.13E−10 | 3.03E−04 | −0.345575797 | 0.345575797 | loss | 0.732989515 | 0.387413718 | NSD1; NSD1 | 37 | 5 |
| cg01308968 | 1.99E−09 | 8.43E−04 | −0.345505963 | 0.345505963 | loss | 0.64543504 | 0.299929076 | IGFBP7-AS1 | 37 | 4 |
| cg15006627 | 1.83E−09 | 7.75E−04 | −0.345453652 | 0.345453652 | loss | 0.719403328 | 0.373949676 | GPRC5A | 37 | 12 |
| cg09069900 | 7.13E−10 | 3.03E−04 | −0.3454528 | 0.3454528 | loss | 0.906289511 | 0.560836712 | F10 | 37 | 13 |
| cg03123541 | 7.13E−10 | 3.03E−04 | −0.345434316 | 0.345434316 | loss | 0.529881745 | 0.184447429 | RWDD3; RWDD3; TMEM56- | 37 | 1 |
| | | | | | | | | RWDD3; RWDD3; RWDD3; | | |
| | | | | | | | | RWDD3; RWDD3; RWDD3 | | |
| cg27143204 | 7.13E−10 | 3.03E−04 | −0.345358887 | 0.345358887 | loss | 0.809378534 | 0.464019647 | CCNO | 37 | 5 |
| cg02124912 | 7.13E−10 | 3.03E−04 | −0.345307783 | 0.345307783 | loss | 0.824297872 | 0.478990088 | GPR37L1 | 37 | 1 |
| cg04218880 | 7.13E−10 | 3.03E−04 | −0.345253491 | 0.345253491 | loss | 0.876496868 | 0.531243376 | | 37 | 1 |
| cg25836232 | 7.13E−10 | 3.03E−04 | −0.345215896 | 0.345215896 | loss | 0.487547268 | 0.142331372 | TUBB6 | 37 | 18 |
| cg04182912 | 7.13E−10 | 3.03E−04 | −0.345083498 | 0.345083498 | loss | 0.642451598 | 0.2973681 | | 37 | 14 |
| cg25221984 | 1.54E−09 | 6.54E−04 | −0.345046329 | 0.345046329 | loss | 0.567655758 | 0.222609429 | NBPF10; LOC10028S142; | 37 | 1 |
| | | | | | | | | CD160; CD160 | | |
| cg03425348 | 8.47E−10 | 3.60E−04 | −0.345044067 | 0.345044067 | loss | 0.661348585 | 0.316304518 | | 37 | 2 |
| cg01668653 | 2.56E−09 | 1.09E−03 | −0.344865536 | 0.344865536 | loss | 0.882137283 | 0.537271747 | | 37 | 6 |
| cg17113856 | 7.13E−10 | 3.03E−04 | −0.344849095 | 0.344849095 | loss | 0.816962066 | 0.472112971 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg17072519 | 7.13E−10 | 3.03E−04 | −0.344739193 | 0.344739193 | loss | 0.794530934 | 0.449791741 | AIFM3; AIFM3; AIFM3; AIFM3 | 37 | 22 |
| cg12043200 | 7.13E−10 | 3.03E−04 | −0.344726163 | 0.344726163 | loss | 0.807460892 | 0.462734729 | | 37 | 14 |
| cg01781662 | 7.13E−10 | 3.03E−04 | −0.344721339 | 0.344721339 | loss | 0.438803468 | 0.094082129 | NTMT1; NTMT1; NTMT1; NTMT1; | 37 | 9 |
| | | | | | | | | NTMT1; NTMT1; NTMT1; | | |
| | | | | | | | | NTMT1; NTMT1; NTMT1 | | |
| cg09395805 | 2.16E−09 | 9.17E−04 | −0.344660874 | 0.344660874 | loss | 0.628650721 | 0.283989847 | | 37 | 6 |
| cg15166039 | 5.39E−09 | 2.29E−03 | −0.344576179 | 0.344576179 | loss | 0.585056991 | 0.240480812 | | 37 | 6 |
| cg03676636 | 7.13E−10 | 3.03E−04 | −0.344516069 | 0.344516069 | loss | 0.787467387 | 0.442951318 | STPG2 | 37 | 4 |
| cg08785317 | 7.13E−10 | 3.03E−04 | −0.344408239 | 0.344408239 | loss | 0.910135798 | 0.565727559 | | 37 | 16 |
| cg18786420 | 7.13E−10 | 3.03E−04 | −0.344403486 | 0.344403486 | loss | 0.700224562 | 0.355821076 | | 37 | 5 |
| cg08605301 | 7.13E−10 | 3.03E−04 | −0.34437199 | 0.34437199 | loss | 0.554983313 | 0.210611324 | WIPF3 | 37 | 7 |
| cg06705862 | 7.77E−10 | 3.30E−04 | −0.344298327 | 0.344298327 | loss | 0.444129483 | 0.099831156 | | 37 | 3 |
| cg13048437 | 7.13E−10 | 3.03E−04 | −0.344177714 | 0.344177714 | loss | 0.815582243 | 0.471404529 | | 37 | 14 |
| cg15448220 | 1.96E−08 | 8.33E−03 | −0.344142123 | 0.344142123 | loss | 0.755397358 | 0.411255235 | SETDB1; SETDB1; SETDB1 | 37 | 1 |
| cg01231141 | 1.83E−09 | 7.75E−04 | −0.344102937 | 0.344102937 | loss | 0.546622904 | 0.202519966 | ADAMTS2; ADAMTS2 | 37 | 5 |
| cg10612560 | 7.13E−10 | 3.03E−04 | −0.34409466 | 0.34409466 | loss | 0.668381019 | 0.324286359 | ALOXE3; ALOXE3 | 37 | 17 |
| cg23323671 | 7.13E−10 | 3.03E−04 | −0.344067068 | 0.344067068 | loss | 0.620223774 | 0.276156706 | STMN1; STMN1; STMN1; STMN1; | 37 | 1 |
| | | | | | | | | MIR3917 | | |
| cg25488547 | 2.16E−09 | 9.17E−04 | −0.343816968 | 0.343816968 | loss | 0.75163027 | 0.407813302 | CHM; CHM | 37 | X |
| cg | | | | | | | | | | |
| 16665310 | 7.77E−10 | 3.30E−04 | −0.343810528 | 0.343810528 | loss | 0.675625734 | 0.331815206 | | 37 | 13 |
| cg25454270 | 7.13E−10 | 3.03E−04 | −0.343755703 | 0.343755703 | loss | 0.686309479 | 0.342553776 | APOA5; APOA5 | 37 | 11 |
| cg04578397 | 7.13E−10 | 3.03E−04 | −0.343643188 | 0.343643188 | loss | 0.776125217 | 0.432482029 | MT1L | 37 | 16 |
| cg12497374 | 7.77E−10 | 3.30E−04 | −0.343400582 | 0.343400582 | loss | 0.5191008 | 0.175700218 | | 37 | 3 |
| cg17900861 | 1.73E−09 | 7.34E−04 | −0.343399273 | 0.343399273 | loss | 0.830796879 | 0.487397606 | | 37 | 15 |
| cg11374335 | 7.13E−10 | 3.03E−04 | −0.343381122 | 0.343381122 | loss | 0.745465781 | 0.402084659 | WTIP | 37 | 19 |
| cg01902605 | 7.13E−10 | 3.03E−04 | −0.343360654 | 0.343360654 | loss | 0.793521766 | 0.450161112 | BHMT2; DMGDH; BHMT2; | 37 | 5 |
| | | | | | | | | DMGDH; DMGDH | | |
| cg14016257 | 7.13E−10 | 3.03E−04 | −0.343281379 | 0.343281379 | loss | 0.591606591 | 0.248325212 | | 37 | 4 |
| cg27651238 | 9.23E−10 | 3.92E−04 | −0.343220122 | 0.343220122 | loss | 0.42037917 | 0.077159048 | | 37 | 12 |
| cg06167719 | 2.35E−09 | 9.98E−04 | −0.343187595 | 0.343187595 | loss | 0.611465655 | 0.268278059 | HLC5; HLC5 | 37 | 21 |
| cg07669403 | 7.13E−10 | 3.03E−04 | −0.343148176 | 0.343148176 | loss | 0.461817015 | 0.118668839 | | 37 | 18 |
| cg15753394 | 7.13E−10 | 3.03E−04 | −0.343112336 | 0.343112336 | loss | 0.494233189 | 0.151120853 | RBM24; RBM24; RBM24 | 37 | 6 |
| cg27295595 | 7.13E−10 | 3.03E−04 | −0.343019627 | 0.343019627 | loss | 0.729296557 | 0.386276929 | | 37 | 16 |
| cg06388647 | 1.01E−09 | 4.27E−04 | −0.342910041 | 0.342910041 | loss | 0.846996064 | 0.504086024 | | 37 | 2 |
| cg09113474 | 7.13E−10 | 3.03E−04 | −0.342886686 | 0.342886686 | loss | 0.616863909 | 0.273977224 | | 37 | 6 |
| cg25263801 | 7.13E−10 | 3.03E−04 | −0.342876399 | 0.342876399 | loss | 0.659368717 | 0.316492318 | GPR85; GPR85; GPR85; GPR85 | 37 | 7 |
| cg09835878 | 7.13E−10 | 3.03E−04 | −0.342844549 | 0.342844549 | loss | 0.879427196 | 0.536582647 | VRTN | 37 | 14 |
| cg18033770 | 7.13E−10 | 3.03E−04 | −0.342741345 | 0.342741345 | loss | 0.774001709 | 0.431260365 | DNLZ; CARD9; CARD9; DNLZ | 37 | 9 |
| cg02760164 | 1.83E−09 | 7.75E−04 | −0.342688096 | 0.342688096 | loss | 0.701885196 | 0.3591971 | PLA2G4D | 37 | 15 |
| cg15255455 | 7.13E−10 | 3.03E−04 | −0.342578139 | 0.342578139 | loss | 0.497252155 | 0.154674016 | PLIN5 | 37 | 19 |
| cg18574813 | 7.13E−10 | 3.03E−04 | −0.342428941 | 0.342428941 | loss | 0.612122653 | 0.269693712 | PDE2A; PDE2A | 37 | 11 |
| cg26230275 | 7.13E−10 | 3.03E−04 | −0.342375083 | 0.342375083 | loss | 0.702737494 | 0.360362412 | S100A10 | 37 | 1 |
| cg04488758 | 1.30E−09 | 5.52E−04 | −0.342324031 | 0.342324031 | loss | 0.673282008 | 0.330957976 | USP44; USP44 | 37 | 12 |
| cg10665961 | 9.23E−10 | 3.92E−04 | −0.342271798 | 0.342271798 | loss | 0.942874951 | 0.600603153 | NTN1 | 37 | 17 |
| cg07090083 | 1.10E−09 | 4.65E−04 | −0.342091842 | 0.342091842 | loss | 0.886257342 | 0.5441655 | PEBP4 | 37 | 8 |
| cg15191795 | 7.13E−10 | 3.03E−04 | −0.341972277 | 0.341972277 | loss | 0.589801547 | 0.247829271 | | 37 | 4 |
| cg15752292 | 7.13E−10 | 3.03E−04 | −0.341847112 | 0.341847112 | loss | 0.685733147 | 0.343886035 | TIMM50 | 37 | 19 |
| cg14232218 | 7.13E−10 | 3.03E−04 | −0.341796009 | 0.341796009 | loss | 0.904663315 | 0.562867306 | | 37 | 9 |
| cg18346398 | 7.13E−10 | 3.03E−04 | −0.341611103 | 0.341611103 | loss | 0.647865779 | 0.306254676 | | 37 | 6 |
| cg06893296 | 8.47E−10 | 3.60E−04 | −0.341548852 | 0.341548852 | loss | 0.774922075 | 0.433373224 | | 37 | 6 |
| cg13430552 | 7.13E−10 | 3.03E−04 | −0.341527106 | 0.341527106 | loss | 0.6517394 | 0.310212294 | | 37 | 2 |
| cg08641963 | 7.13E−10 | 3.03E−04 | −0.34139479 | 0.34139479 | loss | 0.519438502 | 0.178043712 | TNNC2 | 37 | 20 |
| cg18973628 | 7.13E−10 | 3.03E−04 | −0.341382898 | 0.341382898 | loss | 0.804950651 | 0.463567753 | LOC643037 | 37 | 11 |
| cg15829969 | 7.13E−10 | 3.03E−04 | −0.341339265 | 0.341339265 | loss | 0.6069882 | 0.265648935 | NBPF8; NBPF8; NBPF8 | 37 | 1 |
| cg22012583 | 7.13E−10 | 3.03E−04 | −0.341299256 | 0.341299256 | loss | 0.545148668 | 0.203849412 | CERS2; CERS2 | 37 | 1 |
| cg16653408 | 7.84E−10 | 3.33E−04 | −0.341245643 | 0.341245643 | loss | 0.799891196 | 0.458645553 | | 37 | 22 |
| cg00424169 | 7.13E−10 | 3.03E−04 | −0.341198494 | 0.341198494 | loss | 0.677366942 | 0.336168447 | TEX26; TEX26-AS1; TEX26- | 37 | 13 |
| | | | | | | | | AS1; TEX26-AS1; TEX26- | | |
| | | | | | | | | AS1 | | |
| cg05501509 | 7.13E−10 | 3.03E−04 | −0.341117833 | 0.341117833 | loss | 0.885182857 | 0.544065024 | | 37 | 17 |
| cg01278041 | 1.68E−09 | 7.12E−04 | −0.341104817 | 0.341104817 | loss | 0.643868511 | 0.302763694 | WWC2-AS2 | 37 | 4 |
| cg14794735 | 1.73E−09 | 7.34E−04 | −0.34109838 | 0.34109838 | loss | 0.82989333 | 0.48879495 | MIR96; MIR183 | 37 | 7 |
| cg16897193 | 1.42E−09 | 6.01E−04 | −0.340974339 | 0.340974339 | loss | 0.666965581 | 0.325991242 | NOVA2 | 37 | 19 |
| cg21053539 | 8.47E−10 | 3.60E−04 | −0.340951194 | 0.340951194 | loss | 0.927221747 | 0.586270553 | LOC401242 | 37 | 6 |
| cg02282631 | 1.68E−09 | 7.12E−04 | −0.340898764 | 0.340898764 | loss | 0.720809528 | 0.379910765 | | 37 | 5 |
| cg14219918 | 7.13E−10 | 3.03E−04 | −0.340682182 | 0.340682182 | loss | 0.82693057 | 0.486248388 | NR5A1 | 37 | 9 |
| cg08409225 | 7.13E−10 | 3.03E−04 | −0.340620589 | 0.340620589 | loss | 0.554978083 | 0.214357494 | NABP2 | 37 | 12 |
| cg17220161 | 7.13E−10 | 3.03E−04 | −0.340435671 | 0.340435671 | loss | 0.476092587 | 0.135656915 | | 37 | 1 |
| cg19489705 | 7.13E−10 | 3.03E−04 | −0.340414674 | 0.340414674 | loss | 0.937562215 | 0.597147541 | | 37 | 5 |
| cg01568522 | 7.13E−10 | 3.03E−04 | −0.340408866 | 0.340408866 | loss | 0.488957013 | 0.148548147 | SLC4A8; SLC4A8; GALNT6 | 37 | 12 |
| cg03765423 | 7.13E−10 | 3.03E−04 | −0.340273492 | 0.340273492 | loss | 0.674809745 | 0.334536253 | | 37 | 3 |
| cg06491573 | 7.13E−10 | 3.03E−04 | −0.340243387 | 0.340243387 | loss | 0.947355734 | 0.607112347 | KLF15 | 37 | 3 |
| cg06846259 | 7.13E−10 | 3.03E−04 | −0.3400982 | 0.3400982 | loss | 0.691864042 | 0.351765841 | POMC; POMC | 37 | 2 |
| cg05003599 | 8.47E−10 | 3.60E−04 | −0.33997524 | 0.33997524 | loss | 0.803139075 | 0.463163835 | FBXO24; FBXO24; FBXO24; | 37 | 7 |
| | | | | | | | | PCOLCE-AS1 | | |
| cg02570888 | 7.13E−10 | 3.03E−04 | −0.339936006 | 0.339936006 | loss | 0.584550747 | 0.244614741 | | 37 | 1 |
| cg14336501 | 7.13E−10 | 3.03E−04 | −0.339790353 | 0.339790353 | loss | 0.833250406 | 0.493460053 | | 37 | 17 |
| cg20068058 | 7.13E−10 | 3.03E−04 | −0.339746654 | 0.339746654 | loss | 0.600216277 | 0.260469624 | CREB3L1 | 37 | 11 |
| cg04981056 | 7.13E−10 | 3.03E−04 | −0.339743465 | 0.339743465 | loss | 0.840407483 | 0.500664018 | SLC51A | 37 | 3 |
| cg20427865 | 7.13E−10 | 3.03E−04 | −0.339715521 | 0.339715521 | loss | 0.874213715 | 0.534498194 | CX3CL1 | 37 | 16 |
| cg19754520 | 6.35E−09 | 2.70E−03 | −0.33958465 | 0.33958465 | loss | 0.726465726 | 0.386881076 | CPN1 | 37 | 10 |
| cg15414773 | 7.13E−10 | 3.03E−04 | −0.339486766 | 0.339486766 | loss | 0.731182655 | 0.391695888 | | 37 | 1 |
| cg17995050 | 7.13E−10 | 3.03E−04 | −0.33943438 | 0.33943438 | loss | 0.506291474 | 0.166857094 | | 37 | 7 |
| cg25969992 | 7.13E−10 | 3.03E−04 | −0.339386092 | 0.339386092 | loss | 0.537186509 | 0.197800418 | CABP1; CABP1; CABP1 | 37 | 12 |
| cg07718903 | 7.13E−10 | 3.03E−04 | −0.339366812 | 0.339366812 | loss | 0.573226553 | 0.233859741 | FERMT1; FERMT1 | 37 | 20 |
| cg00880984 | 7.13E−10 | 3.03E−04 | −0.339308787 | 0.339308787 | loss | 0.552608787 | 0.2133 | IBTK | 37 | 6 |
| cg14295062 | 7.13E−10 | 3.03E−04 | −0.33912696 | 0.33912696 | loss | 0.647901472 | 0.308774512 | ENTPD2; ENTPD2 | 37 | 9 |
| cg24168538 | 1.68E−09 | 7.12E−04 | −0.339018854 | 0.339018854 | loss | 0.73847636 | 0.399457506 | | 37 | 4 |
| cg09362608 | 7.13E−10 | 3.03E−04 | −0.33893939 | 0.33893939 | loss | 0.532064408 | 0.193125018 | | 37 | 4 |
| cg00509187 | 1.54E−09 | 6.54E−04 | −0.338914104 | 0.338914104 | loss | 0.792746698 | 0.453832594 | ENOX1; ENOX1 | 37 | 13 |
| cg08873792 | 7.77E−10 | 3.30E−04 | −0.338879412 | 0.338879412 | loss | 0.746355536 | 0.407476124 | | 37 | 6 |
| cg20200200 | 7.13E−10 | 3.03E−04 | −0.338816354 | 0.338816354 | loss | 0.68692373 | 0.348107376 | KLHDC7A | 37 | 1 |
| cg05403241 | 7.13E−10 | 3.03E−04 | −0.338686845 | 0.338686845 | loss | 0.542463309 | 0.203776465 | | 37 | 2 |
| cg23251248 | 9.23E−10 | 3.92E−04 | −0.338649382 | 0.338649382 | loss | 0.625103606 | 0.286454224 | TBPL2; TBPL2 | 37 | 14 |
| cg01652244 | 7.77E−10 | 3.30E−04 | −0.338575309 | 0.338575309 | loss | 0.837329732 | 0.498754424 | DPPA5; DPPA5 | 37 | 6 |
| cg19959917 | 7.13E−10 | 3.03E−04 | −0.33852997 | 0.33852997 | loss | 0.598375558 | 0.259845588 | CXCL12; CXCL12; CXCL12; | 37 | 10 |
| | | | | | | | | CXCL12; CXCL12 | | |
| cg07608867 | 7.13E−10 | 3.03E−04 | −0.338506793 | 0.338506793 | loss | 0.762220523 | 0.423713729 | | 37 | 10 |
| cg21790715 | 7.13E−10 | 3.03E−04 | −0.338502595 | 0.338502595 | loss | 0.533105972 | 0.194603376 | | 37 | 2 |
| cg07189222 | 9.34E−10 | 3.97E−04 | −0.338230651 | 0.338230651 | loss | 0.669781298 | 0.331550647 | | 37 | 6 |
| cg08327367 | 1.99E−09 | 8.43E−04 | −0.338212076 | 0.338212076 | loss | 0.898467058 | 0.560254982 | LRP4 | 37 | 11 |
| cg12751521 | 7.13E−10 | 3.03E−04 | −0.338187098 | 0.338187098 | loss | 0.406511658 | 0.068324561 | | 37 | 12 |
| cg02007567 | 7.13E−10 | 3.03E−04 | −0.338150675 | 0.338150675 | loss | 0.642318945 | 0.304168271 | TVP23A; TVP23A | 37 | 16 |
| cg13663855 | 7.13E−10 | 3.03E−04 | −0.338115746 | 0.338115746 | loss | 0.651436923 | 0.313321176 | | 37 | 14 |
| cg15076318 | 1.01E−09 | 4.27E−04 | −0.338100571 | 0.338100571 | loss | 0.537634813 | 0.199534242 | | 37 | 6 |
| cg14624145 | 7.13E−10 | 3.03E−04 | −0.338014629 | 0.338014629 | loss | 0.547337217 | 0.209322588 | | 37 | 17 |
| cg15865827 | 7.13E−10 | 3.03E−04 | −0.337952095 | 0.337952095 | loss | 0.697114866 | 0.359162771 | PSMA8; PSMA8; PSMA8 | 37 | 18 |
| cg11203293 | 8.86E−09 | 3.76E−03 | −0.337715207 | 0.337715207 | loss | 0.686887294 | 0.349172088 | | 37 | 13 |
| cg09361966 | 7.13E−10 | 3.03E−04 | −0.337662002 | 0.337662002 | loss | 0.77292316 | 0.435261159 | | 37 | 1 |
| cg20112159 | 7.13E−10 | 3.03E−04 | −0.337597418 | 0.337597418 | loss | 0.569430936 | 0.231833518 | PTPRQ | 37 | 12 |
| cg14334161 | 7.13E−10 | 3.03E−04 | −0.337586209 | 0.337586209 | loss | 0.733897262 | 0.396311053 | NODAL; NODAL | 37 | 10 |
| cg17105755 | 7.13E−10 | 3.03E−04 | −0.33749158 | 0.33749158 | loss | 0.441780898 | 0.104289318 | M1AP; M1AP; M1AP; M1AP; | 37 | 2 |
| | | | | | | | | M1AP; M1AP | | |
| cg07870378 | 7.13E−10 | 3.03E−04 | −0.337306751 | 0.337306751 | loss | 0.812466645 | 0.475159894 | UBALD1 | 37 | 16 |
| cg23897302 | 7.13E−10 | 3.03E−04 | −0.337298798 | 0.337298798 | loss | 0.595533792 | 0.258234994 | AMN | 37 | 14 |
| cg17870909 | 7.13E−10 | 3.03E−04 | −0.337284234 | 0.337284234 | loss | 0.803409534 | 0.4661253 | FTCD; FTCD | 37 | 21 |
| cg15083775 | 7.13E−10 | 3.03E−04 | −0.33723477 | 0.33723477 | loss | 0.876577464 | 0.539342694 | | 37 | 17 |
| cg13374701 | 7.13E−10 | 3.03E−04 | −0.33720082 | 0.33720082 | loss | 0.978473896 | 0.641273076 | SFN | 37 | 1 |
| cg08597839 | 7.13E−10 | 3.03E−04 | −0.337135772 | 0.337135772 | loss | 0.490016521 | 0.152880749 | | 37 | 1 |
| cg16347256 | 5.81E−08 | 2.47E−02 | −0.337093341 | 0.337093341 | loss | 0.865006935 | 0.527913594 | C1orf168 | 37 | 1 |
| cg26302230 | 7.13E−10 | 3.03E−04 | −0.337038313 | 0.337038313 | loss | 0.676576577 | 0.339538265 | WNT6 | 37 | 2 |
| cg13808012 | 7.13E−10 | 3.03E−04 | −0.337014517 | 0.337014517 | loss | 0.592687317 | 0.2556728 | LOC339529 | 37 | 1 |
| cg01164574 | 9.23E−10 | 3.92E−04 | −0.337002073 | 0.337002073 | loss | 0.791696296 | 0.454694224 | | 37 | 21 |
| cg10923408 | 7.13E−10 | 3.03E−04 | −0.336925426 | 0.336925426 | loss | 0.589515649 | 0.252590224 | TEX26; TEX26-AS1; TEX26- | 37 | 13 |
| | | | | | | | | AS1; TEX26-AS1; TEX26- | | |
| | | | | | | | | AS1 | | |
| cg06520296 | 8.47E−10 | 3.60E−04 | −0.336861161 | 0.336861161 | loss | 0.575147249 | 0.238286088 | | 37 | 8 |
| cg03080147 | 7.13E−10 | 3.03E−04 | −0.336855788 | 0.336855788 | loss | 0.6872704 | 0.350414612 | MTMR7 | 37 | 8 |
| cg24146100 | 1.01E−09 | 4.27E−04 | −0.33678764 | 0.33678764 | loss | 0.687116123 | 0.350328482 | DOCK9; DOCK9 | 37 | 13 |
| cg05822532 | 1.89E−09 | 8.01E−04 | −0.336754775 | 0.336754775 | loss | 0.402659765 | 0.065904991 | ELN; ELN; ELN; ELN; ELN; ELN; | 37 | 7 |
| | | | | | | | | ELN; ELN; ELN; ELN; ELN; | | |
| | | | | | | | | ELN; ELN | | |
| cg25078026 | 7.13E−10 | 3.03E−04 | −0.336752196 | 0.336752196 | loss | 0.80960856 | 0.472856365 | LOC401242 | 37 | 6 |
| cg14981253 | 7.13E−10 | 3.03E−04 | −0.336731993 | 0.336731993 | loss | 0.613185774 | 0.276453781 | RBAK-RBAKDN; RBAKDN | 37 | 7 |
| cg17816637 | 7.13E−10 | 3.03E−04 | −0.336727978 | 0.336727978 | loss | 0.593028221 | 0.256300242 | SLC7A4 | 37 | 22 |
| cg13802736 | 7.13E−10 | 3.03E−04 | −0.336684862 | 0.336684862 | loss | 0.803869779 | 0.467184918 | | 37 | 1 |
| cg07207652 | 9.23E−10 | 3.92E−04 | −0.336655129 | 0.336655129 | loss | 0.780510123 | 0.443854994 | | 37 | 17 |
| cg06714761 | 7.13E−10 | 3.03E−04 | −0.336611843 | 0.336611843 | loss | 0.651452949 | 0.314841106 | | 37 | 1 |
| cg19593741 | 8.55E−08 | 3.63E−02 | −0.336538939 | 0.336538939 | loss | 0.803015398 | 0.466476459 | VWA7 | 37 | 6 |
| cg15671083 | 2.78E−09 | 1.18E−03 | −0.336527893 | 0.336527893 | loss | 0.879217875 | 0.542689982 | UCN3 | 37 | 10 |
| cg05998486 | 7.13E−10 | 3.03E−04 | −0.33645909 | 0.33645909 | loss | 0.499241002 | 0.162781912 | KCNH2; KCNH2 | 37 | 7 |
| cg26802194 | 1.32E−08 | 5.59E−03 | −0.336454607 | 0.336454607 | loss | 0.67052516 | 0.334070553 | CNGB1; CNGB1 | 37 | 16 |
| cg03604322 | 2.56E−09 | 1.09E−03 | −0.336453735 | 0.336453735 | loss | 0.618889938 | 0.282436203 | SLC5A1 | 37 | 22 |
| cg21535947 | 7.13E−10 | 3.03E−04 | −0.336343852 | 0.336343852 | loss | 0.448277853 | 0.111934001 | NUPL1; NUPL1 | 37 | 13 |
| cg11092486 | 1.54E−09 | 6.54E−04 | −0.336305773 | 0.336305773 | loss | 0.588011779 | 0.251706006 | | 37 | 6 |
| cg25224330 | 2.48E−08 | 1.05E−02 | −0.336070815 | 0.336070815 | loss | 0.900990721 | 0.564919906 | | 37 | 7 |
| cg14244636 | 1.01E−09 | 4.27E−04 | −0.336062598 | 0.336062598 | loss | 0.670363757 | 0.334301159 | | 37 | 10 |
| cg05950212 | 7.13E−10 | 3.03E−04 | −0.336000295 | 0.336000295 | loss | 0.618557966 | 0.282557671 | HIST2H2AB | 37 | 1 |
| cg10424681 | 7.13E−10 | 3.03E−04 | −0.335974487 | 0.335974487 | loss | 0.486853675 | 0.150879189 | C6orf201; FAM217A; FAM217A; | 37 | 6 |
| | | | | | | | | C6orf201; C6orf201 | | |
| cg05135729 | 7.77E−10 | 3.30E−04 | −0.335930355 | 0.335930355 | loss | 0.776354302 | 0.440423947 | | 37 | X |
| cg20270653 | 1.30E−09 | 5.52E−04 | −0.335921301 | 0.335921301 | loss | 0.735661766 | 0.399740465 | WBSCR17 | 37 | 7 |
| cg05631230 | 8.47E−10 | 3.60E−04 | −0.335878754 | 0.335878754 | loss | 0.546047594 | 0.210168841 | | 37 | 11 |
| cg17091577 | 7.13E−10 | 3.03E−04 | −0.335866442 | 0.335866442 | loss | 0.489949187 | 0.154082745 | DDR1; DDR1; DDR1 | 37 | 6 |
| cg06634862 | 7.13E−10 | 3.03E−04 | −0.335830888 | 0.335830888 | loss | 0.410997913 | 0.075167025 | LNX1; LNX1; LNX1-AS2 | 37 | 4 |
| cg00872984 | 1.19E−09 | 5.07E−04 | −0.335787565 | 0.335787565 | loss | 0.521314351 | 0.185526786 | TNXB | 37 | 6 |
| cg13305951 | 7.13E−10 | 3.03E−04 | −0.335770753 | 0.335770753 | loss | 0.672833836 | 0.337063082 | RNF112 | 37 | 17 |
| cg02910281 | 7.13E−10 | 3.03E−04 | −0.335753028 | 0.335763028 | loss | 0.856072492 | 0.520309465 | RHBDL2; RHBDL2 | 37 | 1 |
| cg19969694 | 7.13E−10 | 3.03E−04 | −0.335701192 | 0.335701192 | loss | 0.936931315 | 0.601230124 | VPS18 | 37 | 15 |
| cg26266427 | 7.77E−10 | 3.30E−04 | −0.335638885 | 0.335638885 | loss | 0.627181326 | 0.291542441 | TNXB | 37 | 6 |
| cg19676181 | 7.13E−10 | 3.03E−04 | −0.335626736 | 0.335626736 | loss | 0.602721272 | 0.267094535 | | 37 | 4 |
| cg14575854 | 7.13E−10 | 3.03E−04 | −0.335586416 | 0.335586416 | loss | 0.81115364 | 0.475567224 | GRB7; GRB7; GRB7; GRB7 | 37 | 17 |
| cg20118840 | 7.13E−10 | 3.03E−04 | −0.335557972 | 0.335557972 | loss | 0.639267055 | 0.303709082 | | 37 | 11 |
| cg08614082 | 7.77E−10 | 3.30E−04 | −0.33554339 | 0.33554339 | loss | 0.85378676 | 0.518243371 | | 37 | 6 |
| cg25123895 | 1.01E−09 | 4.27E−04 | −0.33553524 | 0.33553524 | loss | 0.605105534 | 0.269570294 | APOB; APOB | 37 | 2 |
| cg27431761 | 7.13E−10 | 3.03E−04 | −0.335516601 | 0.335516601 | loss | 0.730785372 | 0.395268771 | | 37 | 11 |
| cg15851278 | 9.23E−10 | 3.92E−04 | −0.335495051 | 0.335495051 | loss | 0.826087387 | 0.490592335 | DNAJB13 | 37 | 11 |
| cg13888070 | 7.13E−10 | 3.03E−04 | −0.335454987 | 0.335454987 | loss | 0.449064979 | 0.113609992 | SEC62; LOC100128164; LOC100128164 | 37 | 3 |
| cg15124926 | 2.48E−08 | 1.05E−02 | −0.335438267 | 0.335438267 | loss | 0.705126749 | 0.369688482 | WTAPP1 | 37 | 11 |
| cg15620114 | 7.13E−10 | 3.03E−04 | −0.335425899 | 0.335425899 | loss | 0.788090487 | 0.452664588 | SLC16A12 | 37 | 10 |
| cg13939809 | 7.13E−10 | 3.03E−04 | −0.335411977 | 0.335411977 | loss | 0.659645025 | 0.324233047 | | 37 | 18 |
| cg19503249 | 8.47E−10 | 3.60E−04 | −0.33537465 | 0.33537465 | loss | 0.827706932 | 0.492332282 | | 37 | 1 |
| cg26684363 | 7.13E−10 | 3.03E−04 | −0.335352007 | 0.335352007 | loss | 0.469965977 | 0.13461397 | | 37 | 7 |
| cg25924274 | 7.13E−10 | 3.03E−04 | −0.335292613 | 0.335292613 | loss | 0.616541666 | 0.281249053 | | 37 | 2 |
| cg20860806 | 2.16E−09 | 9.17E−04 | −0.335179029 | 0.335179029 | loss | 0.735475111 | 0.400296082 | | 37 | 15 |
| cg02696067 | 2.16E−09 | 9.17E−04 | −0.335124678 | 0.335124678 | loss | 0.708494866 | 0.373370188 | DDR1; DDR1 | 37 | 6 |
| cg08560952 | 7.13E−10 | 3.03E−04 | −0.335115876 | 0.335115876 | loss | 0.83903397 | 0.503918094 | | 37 | 16 |
| cg17889682 | 2.30E−08 | 9.75E−03 | −0.335108911 | 0.335108911 | loss | 0.696202328 | 0.361093428 | DYNC1I1; DYNC1I1; DYNC1I1; | 37 | 7 |
| | | | | | | | | DYNC1I1; DYNC1I1 | | |
| cg14518658 | 7.13E−10 | 3.03E−04 | −0.335106144 | 0.335106144 | loss | 0.463812019 | 0.128705875 | | 37 | 22 |
| cg06390079 | 7.77E−10 | 3.30E−04 | −0.335092012 | 0.335092012 | loss | 0.841038153 | 0.505946141 | ALDH3A1; ALDH3A1; ALDH3A1; | 37 | 17 |
| | | | | | | | | ALDH3A1; ALDH3A1 | | |
| cg14938587 | 7.77E−10 | 3.30E−04 | 0.335086609 | 0.335086609 | gain | 0.195434968 | 0.530521576 | | 37 | 7 |
| cg11521079 | 7.13E−10 | 3.03E−04 | −0.335079381 | 0.335079381 | loss | 0.573525881 | 0.2384465 | | 37 | 17 |
| cg08867399 | 7.77E−10 | 3.30E−04 | −0.335058273 | 0.335058273 | loss | 0.710989232 | 0.375930959 | HS6ST1 | 37 | 2 |
| cg03146625 | 7.13E−10 | 3.03E−04 | −0.335057239 | 0.335057239 | loss | 0.601003568 | 0.265946329 | HOXC4; HOXC4 | 37 | 12 |
| cg03381704 | 1.32E−08 | 5.59E−03 | −0.335008157 | 0.335008157 | loss | 0.739284092 | 0.404275935 | LINC00189 | 37 | 21 |
| cg21024112 | 7.13E−10 | 3.03E−04 | −0.335007481 | 0.335007481 | loss | 0.711458881 | 0.3764514 | | 37 | 6 |
| cg11632685 | 7.13E−10 | 3.03E−04 | −0.335007117 | 0.335007117 | loss | 0.84763817 | 0.512631053 | LOC284798; LOC284798; LOC284798; | 37 | 20 |
| | | | | | | | | LOC284798 | | |
| cg16783571 | 7.13E−10 | 3.03E−04 | −0.334933497 | 0.334933497 | loss | 0.822309938 | 0.487376441 | DPPA5 | 37 | 6 |
| cg09062638 | 7.13E−10 | 3.03E−04 | −0.334918762 | 0.334918762 | loss | 0.914506274 | 0.579587512 | | 37 | 1 |
| cg09735896 | 7.13E−10 | 3.03E−04 | −0.334890352 | 0.334890352 | loss | 0.883418864 | 0.548528512 | KRT12 | 37 | 17 |
| cg18882670 | 1.01E−09 | 4.27E−04 | −0.33485532 | 0.33485532 | loss | 0.887226679 | 0.552371359 | | 37 | X |
| cg18762806 | 7.13E−10 | 3.03E−04 | −0.334797158 | 0.334797158 | loss | 0.61239977 | 0.277602612 | | 37 | 8 |
| cg00290607 | 1.54E−08 | 6.56E−03 | −0.334795425 | 0.334795425 | loss | 0.64121707 | 0.306421645 | DOC2GP | 37 | 11 |
| cg10376827 | 7.13E−10 | 3.03E−04 | −0.334635602 | 0.334635602 | loss | 0.601895891 | 0.267260288 | ADIRF; AGAP11 | 37 | 10 |
| cg06856687 | 7.13E−10 | 3.03E−04 | −0.334621223 | 0.334621223 | loss | 0.614664164 | 0.280042941 | CDH22 | 37 | 20 |
| cg16419584 | 7.13E−10 | 3.03E−04 | −0.334582031 | 0.334582031 | loss | 0.558918619 | 0.224336588 | | 37 | 10 |
| cg22215678 | 7.13E−10 | 3.03E−04 | −0.334476415 | 0.334476415 | loss | 0.732019268 | 0.397542853 | SNRPD1 | 37 | 18 |
| cg11830800 | 3.97E−08 | 1.69E−02 | −0.334466698 | 0.334466698 | loss | 0.562449745 | 0.227983047 | CNTN1; CNTN1 | 37 | 12 |
| cg08918020 | 7.13E−10 | 3.03E−04 | −0.334378419 | 0.334378419 | loss | 0.586230225 | 0.251851806 | COX4I2 | 37 | 20 |
| cg21583668 | 7.13E−10 | 3.03E−04 | −0.33424751 | 0.33424751 | loss | 0.708390357 | 0.374142847 | MON1A; MON1A | 37 | 3 |
| cg27539986 | 7.13E−10 | 3.03E−04 | −0.334236771 | 0.334236771 | loss | 0.428666142 | 0.094429371 | KLHL21 | 37 | 1 |
| cg23779495 | 7.13E−10 | 3.03E−04 | −0.334224635 | 0.334224635 | loss | 0.908365117 | 0.574140482 | LOC401242 | 37 | 6 |
| cg00050402 | 7.13E−10 | 3.03E−04 | −0.334195351 | 0.334195351 | loss | 0.506008147 | 0.171812796 | | 37 | 7 |
| cg02358323 | 7.13E−10 | 3.03E−04 | −0.33417005 | 0.33417005 | loss | 0.715547679 | 0.381377629 | | 37 | 11 |
| cg12905085 | 7.13E−10 | 3.03E−04 | −0.334103738 | 0.334103738 | loss | 0.487040996 | 0.152937258 | | 37 | 1 |
| cg06340713 | 1.19E−09 | 5.07E−04 | −0.333997079 | 0.333997079 | loss | 0.700472596 | 0.366475518 | CHM; CHM | 37 | X |
| cg07939626 | 7.13E−10 | 3.03E−04 | −0.333982321 | 0.333982321 | loss | 0.664742021 | 0.3307597 | DDR1; DDR1; DDR1 | 37 | 6 |
| cg03125400 | 7.13E−10 | 3.03E−04 | −0.333885208 | 0.333885208 | loss | 0.698497849 | 0.364612641 | | 37 | 2 |
| cg19140429 | 4.21E−09 | 1.79E−03 | −0.333855444 | 0.333855444 | loss | 0.559609149 | 0.225753705 | | 37 | 17 |
| cg21683069 | 1.01E−09 | 4.27E−04 | −0.333830353 | 0.333830353 | loss | 0.884388842 | 0.550558488 | NBPF8; NBPF8; NBPF8 | 37 | 1 |
| cg24601559 | 7.13E−10 | 3.03E−04 | −0.333776717 | 0.333776717 | loss | 0.711244258 | 0.377467541 | | 37 | 2 |
| cg22343877 | 7.13E−10 | 3.03E−04 | −0.333634038 | 0.333634038 | loss | 0.852248674 | 0.518614635 | | 37 | 1 |
| cg01300914 | 1.30E−09 | 5.52E−04 | −0.333632295 | 0.333632295 | loss | 0.866058283 | 0.532425988 | | 37 | 6 |
| cg03792783 | 7.13E−10 | 3.03E−04 | −0.333596322 | 0.333596322 | loss | 0.528178387 | 0.194582065 | | 37 | 2 |
| cg01501896 | 7.13E−10 | 3.03E−04 | −0.333567128 | 0.333567128 | loss | 0.840046181 | 0.506479053 | KAT2A; HSPB9 | 37 | 17 |
| cg25428553 | 7.13E−10 | 3.03E−04 | −0.333421428 | 0.333421428 | loss | 0.380036319 | 0.046614891 | | 37 | 2 |
| cg21854759 | 7.13E−10 | 3.03E−04 | −0.333353113 | 0.333353113 | loss | 0.868745677 | 0.535392565 | | 37 | 1 |
| cg12860635 | 7.84E−10 | 3.33E−04 | −0.333346568 | 0.333346568 | loss | 0.832988556 | 0.499641988 | | 37 | 6 |
| cg13290149 | 7.13E−10 | 3.03E−04 | −0.333253515 | 0.333253515 | loss | 0.779291268 | 0.446037753 | RBBP8NL; RBBP8NL | 37 | 20 |
| cg05563558 | 7.13E−10 | 3.03E−04 | −0.333194076 | 0.333194076 | loss | 0.618625853 | 0.285431776 | | 37 | 12 |
| cg24591835 | 1.73E−09 | 7.34E−04 | −0.332895269 | 0.332895269 | loss | 0.706556038 | 0.373760769 | | 37 | 5 |
| cg18322510 | 8.47E−10 | 3.60E−04 | −0.332857323 | 0.332857323 | loss | 0.879408675 | 0.546551353 | LGALS8; LGALS8; LGALS8; LGALS8; | 37 | 1 |
| | | | | | | | | IGALS8-AS1 | | |
| cg10825234 | 1.42E−09 | 6.01E−04 | −0.332795935 | 0.332795935 | loss | 0.861005017 | 0.528209082 | | 37 | 4 |
| cg23433008 | 1.68E−09 | 7.12E−04 | −0.332774696 | 0.332774696 | loss | 0.822644796 | 0.4898701 | RASGEF1C | 37 | 5 |
| cg01628528 | 1.01E−09 | 4.27E−04 | −0.332590434 | 0.332590434 | loss | 0.907262745 | 0.574672312 | C1QTNF9 | 37 | 13 |
| cg20964304 | 7.13E−10 | 3.03E−04 | −0.332570075 | 0.332570075 | loss | 0.563276775 | 0.2307067 | PKNOX2 | 37 | 11 |
| cg22382854 | 7.13E−10 | 3.03E−04 | −0.332553319 | 0.332553319 | loss | 0.759456313 | 0.426902994 | | 37 | 17 |
| cg08166631 | 7.13E−10 | 3.03E−04 | −0.332447664 | 0.332447664 | loss | 0.840837528 | 0.508389865 | | 37 | 1 |
| cg23926439 | 1.42E−09 | 6.01E−04 | −0.332359769 | 0.332359769 | loss | 0.566774525 | 0.234414755 | | 37 | 1 |
| cg00088688 | 1.83E−09 | 7.75E−04 | −0.332346975 | 0.332346975 | loss | 0.676090092 | 0.343743118 | | 37 | 6 |
| cg17329164 | 7.13E−10 | 3.03E−04 | −0.332325028 | 0.332325028 | loss | 0.43975154 | 0.107426511 | PPT2; PPT2; PPT2; LOC100507547; | 37 | 6 |
| | | | | | | | | LOC100507547; LOC100507547; | | |
| | | | | | | | | LOC100507547; | | |
| | | | | | | | | PPT2-EGFL8 | | |
| cg16510654 | 7.13E−10 | 3.03E−04 | −0.332308513 | 0.332308513 | loss | 0.873591719 | 0.541283206 | C1orf64 | 37 | 1 |
| cg02962952 | 7.13E−10 | 3.03E−04 | −0.33226521 | 0.33226521 | loss | 0.737053945 | 0.404788735 | | 37 | 17 |
| cg12526471 | 1.73E−09 | 7.34E−04 | −0.33225771 | 0.33225771 | loss | 0.593054428 | 0.260796719 | ZBTB16 | 37 | 11 |
| cg18630644 | 7.13E−10 | 3.03E−04 | −0.332226399 | 0.332226399 | loss | 0.898867181 | 0.566640732 | C6orf201; FAM217A; FAM217A; | 37 | 6 |
| | | | | | | | | C6orf201; C6orf201 | | |
| cg11598935 | 7.13E−10 | 3.03E−04 | −0.332168206 | 0.332168206 | loss | 0.534490789 | 0.202322582 | BMP7 | 37 | 20 |
| cg19421125 | 7.13E−10 | 3.03E−04 | −0.332091261 | 0.332091261 | loss | 0.445538085 | 0.113446824 | LAG3 | 37 | 12 |
| cg08863620 | 7.13E−10 | 3.03E−04 | −0.332057011 | 0.332057011 | loss | 0.464931045 | 0.132874034 | | 37 | 10 |
| cg06238004 | 8.80E−09 | 3.74E−03 | −0.331934197 | 0.331934197 | loss | 0.866421474 | 0.534487276 | | 37 | 17 |
| cg07671036 | 2.68E−09 | 1.14E−03 | −0.331912499 | 0.331912499 | loss | 0.899265774 | 0.567353275 | PRSS42; PRSS42 | 37 | 3 |
| cg03951833 | 7.13E−10 | 3.03E−04 | −0.331859155 | 0.331859155 | loss | 0.569216485 | 0.237357329 | NBPF8; NBPF8; NBPF8 | 37 | 1 |
| cg04160749 | 2.16E−09 | 9.17E−04 | −0.331789397 | 0.331789397 | loss | 0.786712321 | 0.454922924 | LOC286177 | 37 | 8 |
| cg14950193 | 1.19E−09 | 5.07E−04 | −0.331765879 | 0.331765879 | loss | 0.509977579 | 0.178211701 | C7orf63; C7orf63 | 37 | 7 |
| cg08930131 | 8.11E−09 | 3.44E−03 | −0.331656281 | 0.331656281 | loss | 0.84797854 | 0.516322259 | COL23A1 | 37 | 5 |
| cg03829542 | 7.13E−10 | 3.03E−04 | −0.331648851 | 0.331648851 | loss | 0.79780554 | 0.466156638 | | 37 | 11 |
| cg03253303 | 4.21E−09 | 1.79E−03 | −0.33159527 | 0.33159527 | loss | 0.708319806 | 0.376724535 | RAB11FIP4 | 37 | 17 |
| cg15656686 | 7.13E−10 | 3.03E−04 | −0.331565131 | 0.331565131 | loss | 0.655809555 | 0.324244424 | DDR1; DDR1; DDR1 | 37 | 6 |
| cg14409029 | 9.54E−09 | 4.05E−03 | −0.33153421 | 0.33153421 | loss | 0.53680767 | 0.205273459 | ZDHHC13; ZDHHC13 | 37 | 11 |
| cg13881108 | 7.13E−10 | 3.03E−04 | −0.331386077 | 0.331386077 | loss | 0.681730525 | 0.350344447 | | 37 | 7 |
| cg16613240 | 1.83E−09 | 7.75E−04 | −0.331309785 | 0.331309785 | loss | 0.651970674 | 0.320660888 | | 37 | 6 |
| cg14221766 | 7.13E−10 | 3.03E−04 | −0.331307197 | 0.331307197 | loss | 0.451318292 | 0.120011095 | | 37 | 9 |
| cg00659878 | 1.43E−08 | 6.06E−03 | −0.331154976 | 0.331154976 | loss | 0.935785153 | 0.604630176 | | 37 | 1 |
| cg23536158 | 7.13E−10 | 3.03E−04 | −0.330964245 | 0.330964245 | loss | 0.903037157 | 0.572072912 | SPIDR; SPIDR; SPIDR; SPIDR | 37 | 8 |
| cg23415434 | 7.84E−10 | 3.33E−04 | −0.330938996 | 0.330938996 | loss | 0.541889225 | 0.210950229 | ARHGEF4; ARHGEF4 | 37 | 2 |
| cg10587449 | 7.13E−10 | 3.03E−04 | −0.330877097 | 0.330877097 | loss | 0.783573415 | 0.452696318 | | 37 | 17 |
| cg22620027 | 7.13E−10 | 3.03E−04 | −0.330863059 | 0.330863059 | loss | 0.840122694 | 0.509259635 | | 37 | 1 |
| cg11359720 | 7.13E−10 | 3.03E−04 | −0.330786555 | 0.330786555 | loss | 0.775045749 | 0.444259194 | | 37 | 21 |
| cg24642820 | 7.13E−10 | 3.03E−04 | −0.330647094 | 0.330647094 | loss | 0.451303898 | 0.120656804 | NUP210 | 37 | 3 |
| cg10197057 | 1.83E−09 | 7.75E−04 | −0.330606458 | 0.330606458 | loss | 0.499020502 | 0.168414044 | | 37 | 1 |
| cg16495975 | 7.13E−10 | 3.03E−04 | −0.33054839 | 0.33054839 | loss | 0.537843766 | 0.207295376 | TMEM114 | 37 | 16 |
| cg26227310 | 7.13E−10 | 3.03E−04 | −0.330479721 | 0.330479721 | loss | 0.783849568 | 0.453369847 | TLX1NB | 37 | 10 |
| cg18210365 | 7.13E−10 | 3.03E−04 | −0.330468582 | 0.330468582 | loss | 0.781564064 | 0.451095482 | RBPMS2 | 37 | 15 |
| cg13356540 | 7.13E−10 | 3.03E−04 | −0.330419363 | 0.330419363 | loss | 0.894978351 | 0.564558988 | CAMTA1 | 37 | 1 |
| cg10382206 | 8.47E−10 | 3.60E−04 | −0.330350224 | 0.330350224 | loss | 0.680734994 | 0.350384771 | | 37 | 1 |
| cg21038703 | 7.13E−10 | 3.03E−04 | −0.330334867 | 0.330334867 | loss | 0.782449532 | 0.452114665 | ASB16 | 37 | 17 |
| cg23661466 | 7.13E−10 | 3.03E−04 | −0.330300201 | 0.330300201 | loss | 0.484764825 | 0.154464624 | | 37 | 10 |
| cg02610813 | 7.13E−10 | 3.03E−04 | −0.330186549 | 0.330186549 | loss | 0.724903502 | 0.394716953 | | 37 | 7 |
| cg00961617 | 7.13E−10 | 3.03E−04 | −0.330163304 | 0.330163304 | loss | 0.801980292 | 0.471815988 | PPP1R1B | 37 | 17 |
| cg19154438 | 7.13E−10 | 3.03E−04 | −0.33012302 | 0.33012302 | loss | 0.653211102 | 0.323088082 | CKM | 37 | 19 |
| cg13688770 | 7.13E−10 | 3.03E−04 | −0.330077542 | 0.330077542 | loss | 0.675826925 | 0.345749382 | ZFYVE28; ZFYVE28; ZFYVE28; | 37 | 4 |
| | | | | | | | | ZFYVE28; ZFYVE28; ZFYVE28 | | |
| cg10140454 | 3.28E−09 | 1.39E−03 | −0.330005771 | 0.330005771 | loss | 0.440933819 | 0.110928048 | RAI1 | 37 | 17 |
| cg27625401 | 7.13E−10 | 3.03E−04 | −0.32999632 | 0.32999632 | loss | 0.786894179 | 0.456897859 | ZSCAN10; ZSCAN10 | 37 | 16 |
| cg17565967 | 7.13E−10 | 3.03E−04 | −0.329879503 | 0.329879503 | loss | 0.853821915 | 0.523942412 | RBBP8NL; RBBP8NL | 37 | 20 |
| cg22849655 | 7.13E−10 | 3.03E−04 | −0.32982241 | 0.32982241 | loss | 0.396928306 | 0.067105896 | PRRT1 | 37 | 6 |
| cg16370398 | 8.47E−10 | 3.60E−04 | −0.329783146 | 0.329783146 | loss | 0.525285145 | 0.195501999 | HOXC4; HOXC4 | 37 | 12 |
| cg20509119 | 1.19E−09 | 5.07E−04 | −0.329782062 | 0.329782062 | loss | 0.689098462 | 0.3593164 | | 37 | X |
| cg17393016 | 2.78E−09 | 1.18E−03 | −0.329781931 | 0.329781931 | loss | 0.620428013 | 0.290646082 | LINC00482 | 37 | 17 |
| cg27422857 | 2.35E−09 | 9.98E−04 | −0.32974205 | 0.32974205 | loss | 0.699192368 | 0.369450318 | | 37 | 2 |
| cg03557725 | 7.13E−10 | 3.03E−04 | −0.329739677 | 0.329739677 | loss | 0.46318644 | 0.133446763 | SNRPF | 37 | 12 |
| cg24661236 | 1.42E−09 | 6.01E−04 | −0.329715948 | 0.329715948 | loss | 0.558378525 | 0.228662576 | PI16; PI16 | 37 | 6 |
| cg21813538 | 7.13E−10 | 3.03E−04 | −0.329712179 | 0.329712179 | loss | 0.695200396 | 0.365488218 | MON1A; MON1A | 37 | 3 |
| cg23737190 | 1.42E−09 | 6.01E−04 | −0.329649273 | 0.329649273 | loss | 0.518604242 | 0.188954969 | PI16; PI16 | 37 | 6 |
| cg00487142 | 1.10E−09 | 4.65E−04 | −0.329634037 | 0.329634037 | loss | 0.569926866 | 0.240292829 | DPPA4; DPPA4 | 37 | 3 |
| cg00813720 | 7.13E−10 | 3.03E−04 | −0.329617576 | 0.329617576 | loss | 0.719304706 | 0.389687129 | | 37 | 5 |
| cg26279372 | 7.13E−10 | 3.03E−04 | −0.329605766 | 0.329605766 | loss | 0.657338955 | 0.327733188 | BEGAIN; BEGAIN | 37 | 14 |
| cg04720348 | 7.13E−10 | 3.03E−04 | −0.329536736 | 0.329536736 | loss | 0.824978442 | 0.495441706 | ARHGEF28; ARHGEF28 | 37 | 5 |
| cg09810089 | 7.13E−10 | 3.03E−04 | −0.32950117 | 0.32960117 | loss | 0.393970013 | 0.064468843 | RD3; RD3 | 37 | 1 |
| cg16530165 | 7.13E−10 | 3.03E−04 | −0.329485077 | 0.329485077 | loss | 0.666796777 | 0.3373117 | NPPC | 37 | 2 |
| cg19558933 | 1.10E−09 | 4.65E−04 | −0.329446329 | 0.329446329 | loss | 0.887660564 | 0.558214235 | | 37 | 11 |
| cg14676272 | 3.88E−09 | 1.65E−03 | −0.329420618 | 0.329420618 | loss | 0.838129047 | 0.508708429 | | 37 | 8 |
| cg16358155 | 8.91E−09 | 3.78E−03 | −0.329368833 | 0.329368833 | loss | 0.81154611 | 0.482177276 | MUC4; MUC4; MUC4 | 37 | 3 |
| cg06840491 | 8.47E−10 | 3.60E−04 | −0.329329724 | 0.329329724 | loss | 0.8208041 | 0.491474376 | GDF2; GDF2 | 37 | 10 |
| cg27170141 | 7.77E−10 | 3.30E−04 | −0.32932267 | 0.32932267 | loss | 0.838716317 | 0.509393647 | CASZ1; CASZ1 | 37 | 1 |
| cg26364871 | 1.19E−09 | 5.07E−04 | −0.32928748 | 0.32928748 | loss | 0.878596609 | 0.549309129 | | 37 | 10 |
| cg08198488 | 3.88E−09 | 1.65E−03 | −0.329262107 | 0.329262107 | loss | 0.820743572 | 0.491481465 | TPM1; TPM1; TPM1; TPM1; | 37 | 15 |
| | | | | | | | | TPM1; TPM1 | | |
| cg00099295 | 7.13E−10 | 3.03E−04 | −0.329237959 | 0.329237959 | loss | 0.443280575 | 0.114042616 | MKRN2; MKRN2 | 37 | 3 |
| cg09974661 | 9.23E−10 | 3.92E−04 | −0.329237881 | 0.329237881 | loss | 0.550984745 | 0.221746865 | COLEC11; COLEC11; COLEC11; | 37 | 2 |
| | | | | | | | | COLEC11; COLEC11; COLEC11 | | |
| cg21545013 | 7.13E−10 | 3.03E−04 | −0.329186329 | 0.329186329 | loss | 0.798552706 | 0.469366376 | | 37 | 1 |
| cg00386405 | 6.35E−09 | 2.70E−03 | −0.329159308 | 0.329159308 | loss | 0.947806485 | 0.618647176 | ZNF536 | 37 | 19 |
| cg00296018 | 7.13E−10 | 3.03E−04 | −0.329119575 | 0.329119575 | loss | 0.784941492 | 0.455821918 | | 37 | 5 |
| cg04123507 | 7.13E−10 | 3.03E−04 | −0.329012685 | 0.329012685 | loss | 0.936028243 | 0.607015559 | KRT86 | 37 | 12 |
| cg13686059 | 1.01E−09 | 4.27E−04 | −0.328924445 | 0.328924445 | loss | 0.840011304 | 0.511086859 | | 37 | 2 |
| cg15196156 | 7.13E−10 | 3.03E−04 | −0.328908947 | 0.328908947 | loss | 0.748838964 | 0.419930018 | | 37 | 1 |
| cg07491676 | 4.29E−08 | 1.82E−02 | −0.328783245 | 0.328783245 | loss | 0.689661862 | 0.360878618 | C15orf27 | 37 | 15 |
| cg07357827 | 7.77E−10 | 3.30E−04 | −0.328732801 | 0.328732801 | loss | 0.502416836 | 0.173684035 | SYN2; TIMP4; TIMP4; SYN2 | 37 | 3 |
| cg22343728 | 4.21E−09 | 1.79E−03 | −0.328654813 | 0.328654813 | loss | 0.66323273 | 0.334577918 | | 37 | 22 |
| cg09760963 | 7.13E−10 | 3.03E−04 | −0.32860909 | 0.32850909 | loss | 0.474482002 | 0.145872912 | RNF175 | 37 | 4 |
| cg16215084 | 7.77E−10 | 3.30E−04 | −0.328599019 | 0.328599019 | loss | 0.692227572 | 0.363628553 | DDR1; DDR1; DDR1 | 37 | 6 |
| cg06454084 | 9.23E−10 | 3.92E−04 | −0.32852663 | 0.32852663 | loss | 0.877466242 | 0.548939612 | DACT3 | 37 | 19 |
| cg00355514 | 7.13E−10 | 3.03E−04 | −0.328506698 | 0.328506698 | loss | 0.774027945 | 0.445521247 | | 37 | 13 |
| cg22500876 | 7.13E−10 | 3.03E−04 | −0.328466877 | 0.328466877 | loss | 0.632022377 | 0.3035555 | | 37 | 7 |
| cg03253268 | 7.13E−10 | 3.03E−04 | −0.328404818 | 0.328404818 | loss | 0.539498436 | 0.211093618 | CRYAA | 37 | 21 |
| cg13946767 | 4.21E−09 | 1.79E−03 | −0.328305638 | 0.328305638 | loss | 0.530935151 | 0.202629513 | S100A13; S100A13; S100A13; | 37 | 1 |
| | | | | | | | | S100A13; S100A13; S100A13; | | |
| | | | | | | | | S100A13; S100A1 | | |
| cg15574301 | 8.47E−10 | 3.60E−04 | −0.328279311 | 0.328279311 | loss | 0.54837983 | 0.220100519 | PDLIM4; PDLIM4 | 37 | 5 |
| cg03540028 | 7.13E−10 | 3.03E−04 | −0.328236366 | 0.328236366 | loss | 0.664334408 | 0.336098041 | RIPK4 | 37 | 21 |
| cg13705830 | 8.47E−10 | 3.60E−04 | −0.328202208 | 0.328202208 | loss | 0.701599691 | 0.373397482 | | 37 | 9 |
| cg27305460 | 7.77E−10 | 3.30E−04 | −0.328200378 | 0.328200378 | loss | 0.62494266 | 0.296742282 | | 37 | 15 |
| cg15946312 | 7.13E−10 | 3.03E−04 | −0.328155277 | 0.328155277 | loss | 0.854286142 | 0.526130865 | | 37 | 5 |
| cg25953930 | 7.77E−10 | 3.30E−04 | −0.3281069 | 0.3281069 | loss | 0.602134753 | 0.274027853 | | 37 | 6 |
| cg08450021 | 3.88E−09 | 1.65E−03 | −0.328095494 | 0.328095494 | loss | 0.582322721 | 0.254227226 | | 37 | 11 |
| cg16968885 | 4.57E−09 | 1.94E−03 | 0.32794534 | 0.32794534 | loss | 0.800012092 | 0.472066753 | COL11A1; COL11A1; COL11A1; | 37 | 1 |
| | | | | | | | | COL11A1 | | |
| cg08328513 | 8.47E−10 | 3.60E−04 | −0.327925565 | 0.327925565 | loss | 0.410618489 | 0.082692924 | BHMT2; DMGDH; DMGDH; | 37 | 5 |
| | | | | | | | | BHMT2; DMGDH; DMGDH; | | |
| | | | | | | | | DMGDH; DMGDH | | |
| cg04863005 | 6.80E−08 | 2.89E−02 | −0.327838633 | 0.327838633 | loss | 0.58432553 | 0.256486898 | TACSTD2; TACSTD2 | 37 | 1 |
| cg05148717 | 7.13E−10 | 3.03E−04 | −0.327794123 | 0.327794123 | loss | 0.927577858 | 0.599783735 | LOC401242 | 37 | 6 |
| cg00590063 | 7.77E−10 | 3.30E−04 | −0.327790466 | 0.327790466 | loss | 0.376370213 | 0.048579748 | B3GNT3 | 37 | 19 |
| cg18269141 | 7.13E−10 | 3.03E−04 | −0.327785478 | 0.327785478 | loss | 0.664471713 | 0.336686235 | PRKCZ; PRKCZ; PRKCZ; PRXCZ | 37 | 1 |
| cg02947450 | 1.89E−09 | 8.02E−04 | −0.327752479 | 0.327752479 | loss | 0.796866304 | 0.469113825 | | 37 | 10 |
| cg27561907 | 9.23E−10 | 3.92E−04 | −0.327746269 | 0.327746269 | loss | 0.789866851 | 0.462120582 | PRSS41 | 37 | 16 |
| cg12845268 | 7.13E−10 | 3.03E−04 | −0.327468921 | 0.327468921 | loss | 0.512054021 | 0.1845851 | | 37 | 10 |
| cg07703976 | 7.77E−10 | 3.30E−04 | −0.327431942 | 0.327431942 | loss | 0.95752363 | 0.630091688 | ABCA13 | 37 | 7 |
| cg27387888 | 7.13E−10 | 3.03E−04 | −0.32742536 | 0.32742536 | loss | 0.98027293 | 0.652847571 | | 37 | 22 |
| cg05573767 | 1.18E−08 | 5.01E−03 | −0.327422202 | 0.327422202 | loss | 0.536111719 | 0.208689518 | RPS6KA2 | 37 | 6 |
| cg05903289 | 6.89E−09 | 2.93E−03 | −0.327326961 | 0.327326961 | loss | 0.604475755 | 0.277148794 | | 37 | 2 |
| cg09313482 | 7.77E−10 | 3.30E−04 | −0.327311933 | 0.327311933 | loss | 0.943287821 | 0.615975888 | | 37 | 8 |
| cg03139204 | 7.13E−10 | 3.03E−04 | −0.32712876 | 0.32712876 | loss | 0.740835119 | 0.413706359 | RGN; RGN; RGN; RGN | 37 | X |
| cg05955675 | 7.77E−10 | 3.30E−04 | −0.327125169 | 0.327125169 | loss | 0.750916645 | 0.423791476 | | 37 | 16 |
| cg01825213 | 7.13E−10 | 3.03E−04 | −0.326890551 | 0.326890551 | loss | 0.619976904 | 0.293086353 | | 37 | 9 |
| cg11906444 | 1.01E−09 | 4.27E−04 | −0.326864011 | 0.326864011 | loss | 0.754901475 | 0.428037465 | CD177 | 37 | 19 |
| cg25390787 | 1.42E−09 | 6.01E−04 | −0.326843473 | 0.326843473 | loss | 0.735034996 | 0.408191524 | CRISP2; CRISP2; CRISP2; CRISP2; | 37 | 6 |
| | | | | | | | | CRISP2; CRISP2; CRISP2; | | |
| | | | | | | | | CRISP2; CRISP2; CRISP2; | | |
| | | | | | | | | CRISP2; CRISP2 | | |
| cg05606376 | 5.16E−09 | 2.19E−03 | −0.32680333 | 0.32680333 | loss | 0.892825965 | 0.566022635 | EGFLAM; EGFLAM; EGFLAM; | 37 | 5 |
| | | | | | | | | EGFLAM | | |
| cg15665630 | 7.13E−10 | 3.03E−04 | −0.326778033 | 0.326778033 | loss | 0.513458762 | 0.186680729 | | 37 | 3 |
| cg17792740 | 7.84E−10 | 3.33E−04 | −0.326749854 | 0.326749854 | loss | 0.698212437 | 0.371462582 | ADIRF; AGAP11 | 37 | 10 |
| cg11097968 | 7.13E−10 | 3.03E−04 | −0.326706566 | 0.326706566 | loss | 0.606034872 | 0.279328306 | | 37 | 14 |
| cg00589002 | 7.13E−10 | 3.03E−04 | −0.326620659 | 0.326620659 | loss | 0.680664847 | 0.354044138 | TP73; TP73; TP73; TP73; TP73; | 37 | 1 |
| | | | | | | | | TP73; TP73; TP73; TP73; | | |
| | | | | | | | | TP73; TP73; TP73 | | |
| cg13569486 | 3.28E−09 | 1.39E−03 | −0.326613918 | 0.326613918 | loss | 0.446372991 | 0.119759072 | LMO1 | 37 | 11 |
| cg05213635 | 2.30E−08 | 9.75E−03 | −0.326611606 | 0.326611606 | loss | 0.589169679 | 0.262558074 | | 37 | 11 |
| cg02066331 | 7.77E−10 | 3.30E−04 | −0.326553835 | 0.326553835 | loss | 0.569321 | 0.242767165 | RBM24; RBM24; RBM24 | 37 | 6 |
| cg27597505 | 1.99E−09 | 8.43E−04 | −0.326514222 | 0.326514222 | loss | 0.701125598 | 0.374511376 | CISTR-ACT | 37 | 12 |
| cg01185080 | 7.13E−10 | 3.03E−04 | −0.326506449 | 0.326506449 | loss | 0.541960496 | 0.215454047 | ZNF710 | 37 | 15 |
| cg04139465 | 8.47E−10 | 3.60E−04 | −0.32638035 | 0.32638035 | loss | 0.467230389 | 0.140850038 | LRRC4B | 37 | 19 |
| cg14223001 | 1.68E−09 | 7.12E−04 | −0.326363107 | 0.326363107 | loss | 0.815166866 | 0.488803759 | | 37 | 11 |
| cg07107113 | 7.13E−10 | 3.03E−04 | −0.326277063 | 0.326277063 | loss | 0.713495187 | 0.387218124 | FBLIM1 | 37 | 1 |
| cg06850787 | 1.21E−09 | 5.15E−04 | −0.326270506 | 0.326270506 | loss | 0.883511012 | 0.557240506 | | 37 | 2 |
| cg23629166 | 9.23E−10 | 3.92E−04 | −0.326175165 | 0.326175165 | loss | 0.808718047 | 0.482542882 | PEX10; PEX10 | 37 | 1 |
| cg13159361 | 7.13E−10 | 3.03E−04 | −0.326164805 | 0.326164805 | loss | 0.723753864 | 0.397589059 | | 37 | 15 |
| cg24786658 | 1.83E−09 | 7.75E−04 | −0.326158177 | 0.326158177 | loss | 0.613890525 | 0.287732347 | GPR85; GPR85; GPR85; GPR85 | 37 | 7 |
| cg16391327 | 2.12E−08 | 9.01E−03 | −0.326106285 | 0.326106285 | loss | 0.624838238 | 0.298731953 | FAM98B; FAM98B | 37 | 15 |
| cg06647332 | 7.13E−10 | 3.03E−04 | −0.32606402 | 0.32606402 | loss | 0.864349949 | 0.538285929 | LOC728637 | 37 | 5 |
| cg00540400 | 7.77E−10 | 3.30E−04 | −0.326042663 | 0.326042663 | loss | 0.750002845 | 0.423960182 | | 37 | 15 |
| cg15398400 | 7.13E−10 | 3.03E−04 | −0.326002754 | 0.326002754 | loss | 0.573098177 | 0.247095424 | C10orf82; C10orf82 | 37 | 10 |
| cg18582362 | 7.84E−10 | 3.33E−04 | −0.325994387 | 0.325994387 | loss | 0.571299852 | 0.245305465 | GTPBP10; GTPBP10 | 37 | 7 |
| cg03850936 | 5.85E−09 | 2.49E−03 | −0.325959472 | 0.325959472 | loss | 0.885348808 | 0.559389335 | | 37 | 6 |
| cg04774597 | 7.13E−10 | 3.03E−04 | −0.325884869 | 0.325884869 | loss | 0.39787246 | 0.071987592 | RPLP1; RPLP1 | 37 | 15 |
| cg06861037 | 7.13E−10 | 3.03E−04 | −0.325859626 | 0.325859626 | loss | 0.753410349 | 0.427550724 | | 37 | 16 |
| cg17765892 | 7.13E−10 | 3.03E−04 | −0.325848109 | 0.325848109 | loss | 0.881086221 | 0.555238112 | GDF6 | 37 | 8 |
| cg06679270 | 3.97E−08 | 1.69E−02 | −0.325727676 | 0.325727676 | loss | 0.6284542 | 0.302726524 | CERS3; CERS3 | 37 | 15 |
| cg00318347 | 7.13E−10 | 3.03E−04 | −0.325685452 | 0.325685452 | loss | 0.640549728 | 0.314864276 | EBF2 | 37 | 8 |
| cg19433435 | 7.13E−10 | 3.03E−04 | −0.32565994 | 0.32565994 | loss | 0.780876381 | 0.455216441 | FEZ1; FEZ1 | 37 | 11 |
| cg24435747 | 2.56E−09 | 1.09E−03 | −0.325629145 | 0.325629145 | loss | 0.605995892 | 0.280366747 | IFNLR1; IFNLR1; IFNLR1 | 37 | 1 |
| cg15371026 | 1.68E−09 | 7.12E−04 | −0.325551222 | 0.325551222 | loss | 0.665482528 | 0.339931306 | | 37 | 4 |
| cg22258713 | 1.01E−09 | 4.27E−04 | −0.325548412 | 0.325548412 | loss | 0.788479936 | 0.462931524 | | 37 | 8 |
| cg14192299 | 8.47E−10 | 3.60E−04 | −0.325509347 | 0.325509347 | loss | 0.837102594 | 0.511593247 | NANOS2 | 37 | 19 |
| cg15837308 | 1.32E−08 | 5.59E−03 | −0.325485695 | 0.325485695 | loss | 0.622083236 | 0.296597541 | | 37 | 6 |
| cg18049154 | 1.19E−09 | 5.07E−04 | −0.325435283 | 0.325435283 | loss | 0.711778883 | 0.3863436 | TSLP; TSLP | 37 | 5 |
| cg22872778 | 8.47E−10 | 3.60E−04 | −0.32538701 | 0.32538701 | loss | 0.970905704 | 0.645518694 | ABCA13 | 37 | 7 |
| cg23390118 | 7.13E−10 | 3.03E−04 | −0.325377889 | 0.325377889 | loss | 0.611283425 | 0.285905535 | CACNA1C; CACNA1C; CACNA1C; | 37 | 12 |
| | | | | | | | | CACNA1C; CACNA1C; | | |
| | | | | | | | | CACNA1C; CACNA1C; CACNA1C; | | |
| | | | | | | | | CACNA1C; CACNA1C; | | |
| | | | | | | | | CACNA1C; CACNA1C; CACNA1C; | | |
| | | | | | | | | CACNA1C; CACNA1C; | | |
| | | | | | | | | CACNA1C; CACNA1C; | | |
| | | | | | | | | CACNA1C; CACNA1C; CACNA1C; | | |
| | | | | | | | | CACNA1C; CACNA1C; | | |
| | | | | | | | | CACNA1C | | |
| cg17976873 | 3.28E−09 | 1.39E−03 | −0.325305123 | 0.325305123 | loss | 0.601851275 | 0.276546153 | REM2 | 37 | 14 |
| cg03904291 | 7.13E−10 | 3.03E−04 | −0.325304692 | 0.325304692 | loss | 0.674408415 | 0.349103724 | ABCG4; ABCG4 | 37 | 11 |
| cg09179916 | 3.28E−09 | 1.39E−03 | −0.325249443 | 0.325249443 | loss | 0.865152013 | 0.539902571 | EGFLAM; EGFLAM; EGFLAM; | 37 | 5 |
| | | | | | | | | EGFLAM | | |
| cg12453228 | 7.13E−10 | 3.03E−04 | −0.32522168 | 0.32522168 | loss | 0.609596498 | 0.284374818 | | 37 | 6 |
| cg25547332 | 7.13E−10 | 3.03E−04 | −0.325214674 | 0.325214674 | loss | 0.886406951 | 0.561192276 | LOC728637; LOC728637 | 37 | 5 |
| cg23687098 | 7.13E−10 | 3.03E−04 | −0.325211375 | 0.325221375 | loss | 0.705980487 | 0.381769112 | | 37 | 2 |
| cg05917111 | 7.13E−10 | 3.03E−04 | −0.325177238 | 0.325177238 | loss | 0.708227732 | 0.383050494 | CHST5; CHST5 | 37 | 16 |
| cg21542078 | 7.13E−10 | 3.03E−04 | −0.325171236 | 0.325171236 | loss | 0.803687483 | 0.478516247 | TRPM4; TRPM4 | 37 | 19 |
| cg10697737 | 7.13E−10 | 3.03E−04 | −0.325074298 | 0.325074298 | loss | 0.524835957 | 0.199761659 | | 37 | 19 |
| cg18646240 | 7.13E−10 | 3.03E−04 | −0.325046435 | 0.325046435 | loss | 0.606373653 | 0.281327218 | RLF | 37 | 1 |
| cg20364512 | 7.13E−10 | 3.03E−04 | −0.325024474 | 0.325024474 | loss | 0.830356768 | 0.505332294 | | 37 | 22 |
| cg19734801 | 1.19E−09 | 5.07E−04 | −0.324999435 | 0.324999435 | loss | 0.9726634 | 0.647663965 | LOC728637 | 37 | 5 |
| cg17016026 | 2.56E−09 | 1.09E−03 | −0.324856242 | 0.324856242 | loss | 0.771058625 | 0.446202382 | | 37 | 2 |
| cg18050844 | 7.13E−10 | 3.03E−04 | −0.324841854 | 0.324841854 | loss | 0.903958925 | 0.579117071 | | 37 | 17 |
| cg09852697 | 7.13E−10 | 3.03E−04 | −0.324819877 | 0.324819877 | loss | 0.78436553 | 0.459545653 | TP73; TP73; TP73; TP73; TP73; | 37 | 1 |
| | | | | | | | | TP73; TP73; TP73; TP73; | | |
| | | | | | | | | TP73; TP73; TP73; TP73 | | |
| cg20367266 | 7.13E−10 | 3.03E−04 | −0.324737925 | 0.324737925 | loss | 0.693809725 | 0.3690718 | TCEAL7 | 37 | X |
| cg19234738 | 7.13E−10 | 3.03E−04 | −0.324724819 | 0.324724819 | loss | 0.495482419 | 0.1707576 | | 37 | 3 |
| cg09183151 | 7.13E−10 | 3.03E−04 | −0.324692818 | 0.324692818 | loss | 0.892333389 | 0.567640571 | | 37 | 5 |
| cg21471580 | 1.54E−09 | 6.54E−04 | −0.324669959 | 0.324669959 | loss | 0.899925553 | 0.575255594 | PIWIL2; PIWIL2 | 37 | 8 |
| cg25447894 | 7.13E−10 | 3.03E−04 | −0.324601545 | 0.324601545 | loss | 0.605568709 | 0.280967165 | CSDC2 | 37 | 22 |
| cg05687834 | 7.13E−10 | 3.03E−04 | −0.324533681 | 0.324533681 | loss | 0.702069845 | 0.377536165 | NUAK1 | 37 | 12 |
| cg09544050 | 1.01E−09 | 4.27E−04 | −0.324464262 | 0.324464262 | loss | 0.562439779 | 0.237975518 | BAI1 | 37 | 8 |
| cg18184975 | 7.13E−10 | 3.03E−04 | −0.32434648 | 0.32434648 | loss | 0.802611521 | 0.478265041 | GFRA4; GFRA4; GFRA4; GFRA4 | 37 | 20 |
| cg12806797 | 7.13E−10 | 3.03E−04 | −0.324333523 | 0.324333523 | loss | 0.554602111 | 0.230268588 | H2AFJ; H2AFJ | 37 | 12 |
| cg24969520 | 7.13E−10 | 3.03E−04 | −0.324274712 | 0.324274712 | loss | 0.578434911 | 0.254160199 | TMEM72-AS1 | 37 | 10 |
| cg22457769 | 7.13E−10 | 3.03E−04 | −0.324170889 | 0.324170889 | loss | 0.579422177 | 0.255251288 | FAM83H-AS1 | 37 | 8 |
| cg01025774 | 7.13E−10 | 3.03E−04 | −0.324169959 | 0.324169959 | loss | 0.579416206 | 0.255246247 | | 37 | 5 |
| cg14281592 | 7.13E−10 | 3.03E−04 | −0.324104493 | 0.324104493 | loss | 0.581596075 | 0.257491582 | RALGPS1; RALGPS1; RALGPS1; | 37 | 9 |
| | | | | | | | | ANGPTL2; RALGPS1 | | |
| cg20358011 | 7.34E−08 | 3.12E−02 | 0.324043929 | 0.324043929 | gain | 0.17295663 | 0.497000559 | KIRREL3; KIRREL3 | 37 | 11 |
| cg06174495 | 7.13E−10 | 3.03E−04 | −0.324012384 | 0.324012384 | loss | 0.695218255 | 0.371205871 | | 37 | 21 |
| cg23651872 | 7.13E−10 | 3.03E−04 | −0.323997378 | 0.323997378 | loss | 0.645041119 | 0.321043741 | TBC1D16; TBC1D16; TBC1D16; | 37 | 17 |
| | | | | | | | | TBC1D16; TBC1D16; TBC1D16; | | |
| | | | | | | | | TBC1D16 | | |
| cg10085138 | 7.13E−10 | 3.03E−04 | −0.323981655 | 0.323981655 | loss | 0.824829338 | 0.500847682 | | 37 | 7 |
| cg18209323 | 7.13E−10 | 3.03E−04 | −0.323964667 | 0.323964667 | loss | 0.493714566 | 0.169749899 | | 37 | 7 |
| cg09533178 | 7.13E−10 | 3.03E−04 | −0.323895463 | 0.323895463 | loss | 0.535834381 | 0.211938918 | PPP1R12C; PPP1R12C | 37 | 19 |
| cg09408902 | 7.13E−10 | 3.03E−04 | −0.323793745 | 0.323793745 | loss | 0.533301962 | 0.209508218 | | 37 | 2 |
| cg24920126 | 1.99E−09 | 8.43E−04 | −0.323792898 | 0.323792898 | loss | 0.797631957 | 0.473839059 | PPP1R3G | 37 | 6 |
| cg20918682 | 7.13E−10 | 3.03E−04 | −0.323757824 | 0.323757824 | loss | 0.6761069 | 0.352349076 | LRRN4; LRRN4 | 37 | 20 |
| cg03846641 | 1.83E−09 | 7.75E−04 | −0.323749466 | 0.323749466 | loss | 0.624902672 | 0.301153206 | SH3RF3; SH3RF3- | 37 | 2 |
| | | | | | | | | AS1; SH3RF3-AS1 | | |
| cg06108383 | 7.13E−10 | 3.03E−04 | −0.323644333 | 0.323644333 | loss | 0.873561568 | 0.549917235 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg05189804 | 7.13E−10 | 3.03E−04 | −0.323629881 | 0.323629881 | loss | 0.719837323 | 0.396207441 | CLMP | 37 | 11 |
| cg02726377 | 7.77E−10 | 3.30E−04 | −0.32358263 | 0.32358263 | loss | 0.596021636 | 0.272439006 | SGMS2 | 37 | 4 |
| cg12671565 | 7.13E−10 | 3.03E−04 | −0.323538268 | 0.323538268 | loss | 0.909661568 | 0.5861233 | CACNA2D4 | 37 | 12 |
| cg10608596 | 1.30E−09 | 5.52E−04 | 0.32348336 | 0.32348336 | gain | 0.293814392 | 0.617297753 | ZNF833P | 37 | 19 |
| cg05156901 | 3.88E−09 | 1.65E−03 | −0.323414475 | 0.323414475 | loss | 0.616036087 | 0.292621612 | CPT1B; CPT1B; CPT1B; CPT1B; | 37 | 22 |
| | | | | | | | | CPT1B; CPT1B; CPT1B; | | |
| | | | | | | | | CPT1B; CHKB-CPT1B | | |
| cg21033965 | 7.13E−10 | 3.03E−04 | −0.323381609 | 0.323381609 | loss | 0.442414681 | 0.119033072 | | 37 | 1 |
| cg10531073 | 7.77E−10 | 3.30E−04 | −0.323316016 | 0.323316016 | loss | 0.775464487 | 0.452148471 | BAIAP2L2 | 37 | 22 |
| cg10441379 | 7.13E−10 | 3.03E−04 | −0.323217133 | 0.323217133 | loss | 0.585131762 | 0.261914629 | BTF3; BTF3 | 37 | 5 |
| cg04136369 | 1.68E−09 | 7.12E−04 | −0.323153329 | 0.323153329 | loss | 0.714140917 | 0.390987588 | HNF1B; HNF1B | 37 | 17 |
| cg07068045 | 7.13E−10 | 3.03E−04 | −0.323146892 | 0.323146892 | loss | 0.838867915 | 0.515721024 | | 37 | 11 |
| cg03221914 | 7.77E−10 | 3.30E−04 | −0.323114462 | 0.323114462 | loss | 0.704598215 | 0.381483753 | HIST1H2AJ | 37 | 6 |
| cg24829409 | 1.54E−09 | 6.54E−04 | −0.323082284 | 0.323082284 | loss | 0.569746442 | 0.246664157 | LINC00588 | 37 | 8 |
| cg21734175 | 7.13E−10 | 3.03E−04 | −0.323061559 | 0.323061559 | loss | 0.644047447 | 0.320985888 | | 37 | 12 |
| cg00056497 | 8.47E−10 | 3.60E−04 | −0.323031708 | 0.323031708 | loss | 0.603146538 | 0.280114829 | LOC399829 | 37 | 10 |
| cg22158992 | 7.13E−10 | 3.03E−04 | −0.322974525 | 0.322974525 | loss | 0.667977625 | 0.3450031 | NKD2; NKD2 | 37 | 5 |
| cg13331196 | 7.13E−10 | 3.03E−04 | −0.32291748 | 0.32291748 | loss | 0.378148457 | 0.055230976 | | 37 | 10 |
| cg11586382 | 7.13E−10 | 3.03E−04 | −0.322867813 | 0.322867813 | loss | 0.821171813 | 0.498304 | PITRM1; PITRM1; PITRM1 | 37 | 10 |
| cg27449258 | 7.13E−10 | 3.03E−04 | −0.322846915 | 0.322846915 | loss | 0.873694568 | 0.550847653 | ADARB1; ADARB1; ADARB1; | 37 | 21 |
| | | | | | | | | ADARB1; SSR4P1; ADARB1; | | |
| | | | | | | | | ADARB1; ADARB1; ADARB1 | | |
| cg24576535 | 7.13E−10 | 3.03E−04 | −0.322808439 | 0.322808439 | loss | 0.396874064 | 0.074065625 | | 37 | 3 |
| cg14836839 | 7.13E−10 | 3.03E−04 | −0.322765935 | 0.322765935 | loss | 0.423060474 | 0.100294538 | | 37 | 17 |
| cg03814644 | 4.97E−09 | 2.11E−03 | −0.322743614 | 0.322743614 | loss | 0.702021832 | 0.379278218 | LINC00473; LINC00473 | 37 | 6 |
| cg07033475 | 1.10E−09 | 4.65E−04 | −0.322677381 | 0.322677381 | loss | 0.652300823 | 0.329623441 | | 37 | 11 |
| cg23812775 | 1.01E−09 | 4.27E−04 | −0.322646636 | 0.322646636 | loss | 0.739917277 | 0.417270641 | GDF2; GDF2 | 37 | 10 |
| cg00147172 | 3.88E−09 | 1.65E−03 | −0.32264274 | 0.32264274 | loss | 0.617744087 | 0.295101347 | | 37 | 22 |
| cg19176553 | 7.13E−10 | 3.03E−04 | −0.322626144 | 0.322626144 | loss | 0.572435368 | 0.249809224 | | 37 | 17 |
| cg20444256 | 7.13E−10 | 3.03E−04 | −0.322606872 | 0.322606872 | loss | 0.625041655 | 0.302434782 | CKM; CKM | 37 | 19 |
| cg19478539 | 7.13E−10 | 3.03E−04 | −0.32245092 | 0.32245092 | loss | 0.920217691 | 0.597766771 | | 37 | 17 |
| cg20366479 | 7.13E−10 | 3.03E−04 | −0.322427867 | 0.322427867 | loss | 0.555571585 | 0.234143718 | IGFBP2 | 37 | 2 |
| cg10875257 | 7.13E−10 | 3.03E−04 | −0.322375025 | 0.322375025 | loss | 0.746857025 | 0.424482 | SCAND3 | 37 | 6 |
| cg15013527 | 7.13E−10 | 3.03E−04 | −0.322353761 | 0.322353761 | loss | 0.545234938 | 0.222881176 | | 37 | 11 |
| cg09676390 | 7.13E−10 | 3.03E−04 | −0.32231794 | 0.32231794 | loss | 0.77134067 | 0.449022729 | ADARB1; ADARB1; ADARB1; | 37 | 21 |
| | | | | | | | | ADARB1; SSR4P1; ADARB1; | | |
| | | | | | | | | ADARB1; ADARB1; ADARB1 | | |
| cg21902394 | 7.13E−10 | 3.03E−04 | −0.322306716 | 0.322306716 | loss | 0.831564545 | 0.509257829 | | 37 | 17 |
| cg02463440 | 7.13E−10 | 3.03E−04 | −0.322280212 | 0.322280212 | loss | 0.943839183 | 0.621558971 | PIWIL2; PIWIL2 | 37 | 8 |
| cg11170318 | 7.13E−10 | 3.03E−04 | −0.32212618 | 0.32212618 | loss | 0.823095657 | 0.500969476 | | 37 | 21 |
| cg14837974 | 7.13E−10 | 3.03E−04 | −0.322110503 | 0.322110503 | loss | 0.822677526 | 0.500567024 | OR6S1 | 37 | 14 |
| cg25845463 | 7.13E−10 | 3.03E−04 | −0.32206635 | 0.32206635 | loss | 0.718155526 | 0.396089176 | PRKCZ; PRKCZ; PRKCZ; PRKCZ | 37 | 1 |
| cg02738086 | 7.13E−10 | 3.03E−04 | −0.322029987 | 0.322029987 | loss | 0.723650623 | 0.401620635 | POLR3H; POLR3H; POLR3H; | 37 | 22 |
| | | | | | | | | POLR3H; POLR3H | | |
| cg19398496 | 7.13E−10 | 3.03E−04 | −0.321780182 | 0.321780182 | loss | 0.7976125 | 0.475832318 | | 37 | 6 |
| cg17872886 | 8.47E−10 | 3.60E−04 | −0.321718617 | 0.321718617 | loss | 0.484402875 | 0.162684258 | COLEC11; COLEC11; CDLEC11; | 37 | 2 |
| | | | | | | | | COLEC11; COLEC11; COLEC11 | | |
| cg20673321 | 7.13E−10 | 3.03E−04 | −0.321716263 | 0.321716263 | loss | 0.668747145 | 0.347030882 | ZNF541 | 37 | 19 |
| cg07855242 | 1.16E−07 | 4.91E−02 | −0.321712891 | 0.321712891 | loss | 0.907035168 | 0.585322276 | MYT1L | 37 | 2 |
| cg19570321 | 7.13E−10 | 3.03E−04 | −0.321710933 | 0.321710933 | loss | 0.517742215 | 0.196031282 | HRASLS5; HRASLS5; HRASLS5; | 37 | 11 |
| | | | | | | | | MIR3680-1; MIR3680-2 | | |
| cg21116077 | 7.13E−10 | 3.03E−04 | −0.321706971 | 0.321706971 | loss | 0.836997883 | 0.515290912 | | 37 | 1 |
| cg22508928 | 1.01E−09 | 4.27E−04 | −0.3216958 | 0.3216958 | loss | 0.8302331 | 0.5085373 | CASZ1; CASZ1 | 37 | 1 |
| cg17239057 | 8.47E−10 | 3.60E−04 | −0.321689365 | 0.321689365 | loss | 0.515219051 | 0.193529686 | | 37 | 1 |
| cg16559243 | 9.23E−10 | 3.92E−04 | −0.321673177 | 0.321673177 | loss | 0.841615436 | 0.519942259 | EGFLAM; EGFLAM; EGFLAM; | 37 | 5 |
| | | | | | | | | EGFLAM | | |
| cg18055623 | 7.77E−10 | 3.30E−04 | −0.321427475 | 0.321427475 | loss | 0.618881881 | 0.297454406 | CACNG8; MIR935 | 37 | 19 |
| cg24569251 | 7.92E−08 | 3.36E−02 | −0.321270511 | 0.321270511 | loss | 0.850753111 | 0.5294826 | | 37 | 6 |
| cg03961510 | 1.19E−09 | 5.07E−04 | −0.3212511 | 0.3212511 | loss | 0.945148511 | 0.623897412 | | 37 | 10 |
| cg00995520 | 7.13E−10 | 3.03E−04 | −0.321177768 | 0.321177768 | loss | 0.667045174 | 0.345867406 | KCNA3 | 37 | 1 |
| cg02486145 | 1.67E−08 | 7.10E−03 | −0.321145316 | 0.321145316 | loss | 0.718099798 | 0.396954482 | | 37 | 2 |
| cg00245465 | 2.12E−08 | 9.01E−03 | −0.321109756 | 0.321109756 | loss | 0.768755374 | 0.447645618 | AICDA | 37 | 12 |
| cg27071797 | 7.13E−10 | 3.03E−04 | −0.321105505 | 0.321105505 | loss | 0.87201137 | 0.550905865 | | 37 | 15 |
| cg00348467 | 7.13E−10 | 3.03E−04 | −0.321045603 | 0.321045603 | loss | 0.423565732 | 0.102520129 | | 37 | 17 |
| cg05526015 | 3.14E−08 | 1.33E−02 | −0.321025835 | 0.321025835 | loss | 0.897110353 | 0.576084518 | | 37 | 6 |
| cg25594486 | 7.13E−10 | 3.03E−04 | −0.320982874 | 0.320982874 | loss | 0.798198962 | 0.477216088 | SHANK1 | 37 | 19 |
| cg22039909 | 7.13E−10 | 3.03E−04 | −0.320972021 | 0.320972021 | loss | 0.785036862 | 0.464064841 | TP73; TP73; TP73; TP73; TP73; | 37 | 1 |
| | | | | | | | | TP73; TP73; TP73; TP73; | | |
| | | | | | | | | TP73; TP73; TP73; TP73 | | |
| cg27126225 | 7.13E−10 | 3.03E−04 | −0.320954533 | 0.320954533 | loss | 0.829532409 | 0.508577876 | TUB; TUB | 37 | 11 |
| cg19717326 | 7.13E−10 | 3.03E−04 | −0.320920114 | 0.320920114 | loss | 0.799410991 | 0.478490876 | MYADM; MYADM; MYADM | 37 | 19 |
| cg22914338 | 9.23E−10 | 3.92E−04 | −0.320896518 | 0.320896518 | loss | 0.919048836 | 0.598152318 | | 37 | 19 |
| cg03998792 | 7.13E−10 | 3.03E−04 | −0.320877825 | 0.320877825 | loss | 0.77295036 | 0.452072535 | | 37 | 11 |
| cg12125332 | 1.73E−09 | 7.34E−04 | −0.32084821 | 0.32084821 | loss | 0.583172604 | 0.262324394 | ZC2HC1C; ZC2HC1C | 37 | 14 |
| cg09320367 | 7.13E−10 | 3.03E−04 | −0.320841437 | 0.320841437 | loss | 0.809284866 | 0.488443429 | | 37 | 1 |
| cg02680487 | 7.13E−10 | 3.03E−04 | −0.320795942 | 0.320795942 | loss | 0.562135377 | 0.241339435 | DDR1; DDR1; DDR1 | 37 | 6 |
| cg02477448 | 7.13E−10 | 3.03E−04 | −0.320740088 | 0.320740088 | loss | 0.892211711 | 0.571471624 | H1F0 | 37 | 22 |
| cg05341260 | 7.13E−10 | 3.03E−04 | −0.320731621 | 0.320731621 | loss | 0.636105315 | 0.315373694 | | 37 | 17 |
| cg14833891 | 1.01E−09 | 4.27E−04 | −0.320670661 | 0.320670661 | loss | 0.927500891 | 0.606830229 | | 37 | 7 |
| cg19975916 | 1.99E−09 | 8.43E−04 | −0.320618477 | 0.320618477 | loss | 0.816468042 | 0.495849565 | HCAR1; HCAR1 | 37 | 12 |
| cg06074123 | 7.13E−10 | 3.03E−04 | −0.320608942 | 0.320608942 | loss | 0.869110736 | 0.548501794 | | 37 | 4 |
| cg15234845 | 7.13E−10 | 3.03E−04 | −0.320589245 | 0.320589245 | loss | 0.721666192 | 0.401076947 | | 37 | 10 |
| cg04636402 | 7.13E−10 | 3.03E−04 | −0.320577151 | 0.320577151 | loss | 0.495202372 | 0.174625221 | NRG2; NRG2; NRG2; NRG2; | 37 | 5 |
| | | | | | | | | NRG2 | | |
| cg01847021 | 7.13E−10 | 3.03E−04 | −0.320567417 | 0.320567417 | loss | 0.83182407 | 0.511256653 | RADIL | 37 | 7 |
| cg00850481 | 1.83E−09 | 7.75E−04 | −0.320566 | 0.320566 | loss | 0.6811051 | 0.3605391 | | 37 | 1 |
| cg19999682 | 7.13E−10 | 3.03E−04 | −0.320431705 | 0.320431705 | loss | 0.855765964 | 0.535334259 | | 37 | 4 |
| cg01467047 | 7.13E−10 | 3.03E−04 | −0.320399007 | 0.320399007 | loss | 0.871022572 | 0.550623565 | FOXL1 | 37 | 16 |
| cg14128068 | 7.13E−10 | 3.03E−04 | −0.320383172 | 0.370383172 | loss | 0.894392408 | 0.574009235 | | 37 | 9 |
| cg16468843 | 7.34E−08 | 3.12E−02 | −0.320356006 | 0.320356006 | loss | 0.810841242 | 0.490485235 | ATP2B2; ATP2B2 | 37 | 3 |
| cg14209784 | 7.13E−10 | 3.03E−04 | −0.320348811 | 0.320348811 | loss | 0.833000323 | 0.512651512 | ADIRF; AGAP11 | 37 | 10 |
| cg08383929 | 7.13E−10 | 3.03E−04 | −0.320264901 | 0.320264901 | loss | 0.799764172 | 0.479499271 | | 37 | 5 |
| cg24942458 | 7.13E−10 | 3.03E−04 | −0.320211258 | 0.320211258 | loss | 0.685699875 | 0.365488618 | | 37 | 19 |
| cg24530234 | 1.73E−09 | 7.34E−04 | −0.320183747 | 0.320183747 | loss | 0.636718172 | 0.316534425 | | 37 | 10 |
| cg11560654 | 2.48E−08 | 1.05E−02 | −0.32006518 | 0.32006518 | loss | 0.754497362 | 0.434432182 | | 37 | 13 |
| cg13507429 | 7.13E−10 | 3.03E−04 | −0.320001633 | 0.320001633 | loss | 0.787656715 | 0.467655082 | KLHDC7A | 37 | 1 |
| cg13796381 | 7.13E−10 | 3.03E−04 | −0.319976903 | 0.319976903 | loss | 0.427977687 | 0.108000784 | PTGDS | 37 | 9 |
| cg10350722 | 6.30E−08 | 2.68E−02 | −0.319929221 | 0.319929221 | loss | 0.864901404 | 0.544972182 | PRMT8 | 37 | 12 |
| cg20758542 | 7.13E−10 | 3.03E−04 | −0.319917571 | 0.319917571 | loss | 0.814430347 | 0.494512776 | IL17RA | 37 | 22 |
| cg19443920 | 7.84E−10 | 3.33E−04 | −0.319863614 | 0.319863614 | loss | 0.82309789 | 0.503234276 | ACLY; ACLY | 37 | 17 |
| cg21890667 | 7.13E−10 | 3.03E−04 | −0.319777889 | 0.319777889 | loss | 0.547302142 | 0.227524253 | HORMAD2 | 37 | 22 |
| cg10068408 | 7.13E−10 | 3.03E−04 | −0.319579336 | 0.319579336 | loss | 0.874061025 | 0.554481688 | SLC22A8; SLC22A8; SLC22A8; | 37 | 11 |
| | | | | | | | | SLC22A8; SLC22A8; SLC22A8; | | |
| | | | | | | | | SLC22A8; SLC22A8 | | |
| cg08350047 | 7.13E−10 | 3.03E−04 | −0.319529062 | 0.319529062 | loss | 0.760495662 | 0.4409656 | | 37 | 16 |
| cg16081457 | 6.89E−09 | 2.93E−03 | −0.319526684 | 0.319526684 | loss | 0.590902202 | 0.271375518 | | 37 | 12 |
| cg23018236 | 7.13E−10 | 3.03E−04 | −0.319489044 | 0.319489044 | loss | 0.575901574 | 0.256412529 | | 37 | 17 |
| cg09856367 | 6.37E−09 | 2.70E−03 | −0.319486924 | 0.319486924 | loss | 0.613539259 | 0.294052335 | | 37 | 3 |
| cg11320749 | 7.13E−10 | 3.03E−04 | −0.319459658 | 0.319469658 | loss | 0.833385111 | 0.513915453 | | 37 | 10 |
| cg05434641 | 9.34E−10 | 3.97E−04 | −0.319396266 | 0.319396266 | loss | 0.567375454 | 0.247979188 | | 37 | 16 |
| cg27648238 | 7.13E−10 | 3.03E−04 | −0.31933895 | 0.31933895 | loss | 0.881028179 | 0.561689229 | | 37 | 8 |
| cg23657179 | 7.13E−10 | 3.03E−04 | −0.31928481 | 0.31928481 | loss | 0.53720514 | 0.217920329 | ZNF503-AS2; ZNF503-AS2 | 37 | 10 |
| cg23367683 | 1.10E−09 | 4.65E−04 | −0.319265302 | 0.319265302 | loss | 0.85530676 | 0.536041459 | TRIM7; TRIM7; TRIM7; TRIM7; | 37 | 5 |
| | | | | | | | | TRIM7; TRIM7 | | |
| cg03630736 | 1.89E−09 | 8.01E−04 | −0.319236144 | 0.319236144 | loss | 0.77186635 | 0.452630206 | SRP68; SRP68; SRP68 | 37 | 17 |
| cg19402371 | 7.13E−10 | 3.03E−04 | −0.319230774 | 0.319230774 | loss | 0.723499121 | 0.404268347 | TMEM190 | 37 | 19 |
| cg21833184 | 4.57E−09 | 1.94E−03 | −0.31922542 | 0.31922542 | loss | 0.689187185 | 0.369961765 | RBAK-RBAKDN; RBAKDN | 37 | 7 |
| cg05216268 | 7.13E−10 | 3.03E−04 | −0.319218112 | 0.319218112 | loss | 0.756096689 | 0.436878576 | PLA2G4E | 37 | 15 |
| cg07168142 | 7.13E−10 | 3.03E−04 | −0.319190772 | 0.319190772 | loss | 0.974454172 | 0.6552634 | KAT2A; KSPB9 | 37 | 17 |
| cg14625581 | 7.13E−10 | 3.03E−04 | −0.319145845 | 0.319145845 | loss | 0.364733953 | 0.045588108 | | 37 | 17 |
| cg10432947 | 7.77E−10 | 3.30E−04 | −0.319130212 | 0.319130212 | loss | 0.638306142 | 0.319175929 | NAT8L | 37 | 4 |
| cg19710238 | 7.13E−10 | 3.03E−04 | −0.319101915 | 0.319101915 | loss | 0.563852809 | 0.244750894 | | 37 | 2 |
| cg27001715 | 1.89E−09 | 8.01E−04 | −0.319086435 | 0.319086435 | loss | 0.602555354 | 0.283468919 | | 37 | 6 |
| cg22549041 | 1.30E−09 | 5.52E−04 | −0.318985928 | 0.318985928 | loss | 0.402471625 | 0.083485696 | CYP1A1 | 37 | 15 |
| cg26952618 | 7.13E−10 | 3.03E−04 | −0.318956745 | 0.318956745 | loss | 0.439203492 | 0.120246748 | TVP23A; TVP23A | 37 | 16 |
| cg14785464 | 2.78E−09 | 1.18E−03 | −0.31888052 | 0.31888052 | loss | 0.507715898 | 0.188835378 | HLCS; HLCS | 37 | 21 |
| cg08819022 | 2.91E−08 | 1.23E−02 | −0.318780448 | 0.318780448 | loss | 0.801604689 | 0.482824241 | | 37 | 22 |
| cg09065876 | 7.13E−10 | 3.03E−04 | −0.318754757 | 0.318754757 | loss | 0.391994979 | 0.073240222 | | 37 | 17 |
| cg12440062 | 7.77E−10 | 3.30E−04 | −0.318746972 | 0.318746972 | loss | 0.666101072 | 0.3473541 | CRISP2; CRISP2; CRISP2; CRISP2; | 37 | 6 |
| | | | | | | | | CRISP2; CRISP2; CRISP2; | | |
| | | | | | | | | CRISP2; CRISP2; CRISP2; | | |
| | | | | | | | | CRISP2; CRISP2 | | |
| cg01332054 | 7.13E−10 | 3.03E−04 | −0.318703459 | 0.318703459 | loss | 0.733308394 | 0.414604935 | STRA6; STRA6; STRA6; STRA6; | 37 | 15 |
| | | | | | | | | STRA6; STRA6; STRAS; STRA6 | | |
| cg00964751 | 7.13E−10 | 3.03E−04 | −0.318667779 | 0.318667779 | loss | 0.737129574 | 0.418461794 | C17orf96 | 37 | 17 |
| cg19112186 | 7.48E−09 | 3.18E−03 | −0.318601876 | 0.318601876 | loss | 0.593246032 | 0.274644156 | CPT1B; CPT1B; CPT1B; CPT1B; | 37 | 22 |
| | | | | | | | | CPT1B; CPT1B; CPT1B; | | |
| | | | | | | | | CPT1B; CHKB-CPT1B | | |
| cg13583088 | 7.13E−10 | 3.03E−04 | −0.318515074 | 0.318515074 | loss | 0.908155721 | 0.589640647 | | 37 | 14 |
| cg11367159 | 7.13E−10 | 3.03E−04 | −0.318465723 | 0.318465723 | loss | 0.455352217 | 0.136886494 | | 37 | 12 |
| cg26784300 | 7.13E−10 | 3.03E−04 | −0.318457693 | 0.318457693 | loss | 0.662894087 | 0.344436394 | | 37 | 2 |
| cg27464846 | 1.42E−09 | 6.01E−04 | −0.318453751 | 0.318453751 | loss | 0.761836609 | 0.443382859 | | 37 | 16 |
| cg19463349 | 1.96E−08 | 8.33E−03 | −0.31825157 | 0.31825157 | loss | 0.699495153 | 0.381243582 | | 37 | 13 |
| cg16689481 | 3.28E−09 | 1.39E−03 | 0.318169297 | 0.318169297 | gain | 0.383002221 | 0.701171518 | TCHH | 37 | 1 |
| cg26875665 | 7.13E−10 | 3.03E−04 | −0.318095799 | 0.318095799 | loss | 0.828803734 | 0.510707935 | NHLH1 | 37 | 1 |
| cg22251148 | 7.13E−10 | 3.03E−04 | −0.318042995 | 0.318042995 | loss | 0.787409036 | 0.469366041 | | 37 | 11 |
| cg03983336 | 1.54E−09 | 6.54E−04 | −0.318026447 | 0.318026447 | loss | 0.852147806 | 0.534121359 | SLC5A7 | 37 | 2 |
| cg15302032 | 8.47E−10 | 3.60E−04 | −0.31799163 | 0.31799163 | loss | 0.657001447 | 0.339009818 | NTM | 37 | 11 |
| cg02207638 | 7.13E−10 | 3.03E−04 | −0.317938754 | 0.317938754 | loss | 0.87781243 | 0.559873676 | FAM83C; FAM83C | 37 | 20 |
| cg11868728 | 7.13E−10 | 3.03E−04 | −0.317907185 | 0.317907185 | loss | 0.863782685 | 0.5458755 | | 37 | 20 |
| cg00989365 | 7.13E−10 | 3.03E−04 | −0.317827873 | 0.317827873 | loss | 0.436513187 | 0.118685314 | | 37 | 14 |
| cg21477262 | 7.13E−10 | 3.03E−04 | −0.317812613 | 0.317812613 | loss | 0.870238908 | 0.552426294 | ETNK1; ETNK1 | 37 | 12 |
| cg27569858 | 7.13E−10 | 3.03E−04 | −0.317792496 | 0.317792496 | loss | 0.75265326 | 0.434860765 | ALX3 | 37 | 1 |
| cg01239922 | 7.13E−10 | 3.03E−04 | −0.317660167 | 0.317660167 | loss | 0.837545532 | 0.519885365 | LRRC14B | 37 | 5 |
| cg00947878 | 1.30E−09 | 5.52E−04 | −0.317623453 | 0.317623453 | loss | 0.691180942 | 0.373557488 | KIFC3; XIFC3; KIFC3; KIFC3 | 37 | 16 |
| cg24268343 | 7.13E−10 | 3.03E−04 | −0.317548519 | 0.317548519 | loss | 0.578599219 | 0.2610507 | KLF8; KLF8 | 37 | X |
| cg20925954 | 7.13E−10 | 3.03E−04 | −0.317536845 | 0.317536845 | loss | 0.564474745 | 0.2469379 | DUSP5 | 37 | 10 |
| cg27314669 | 7.13E−10 | 3.03E−04 | −0.317361954 | 0.317361954 | loss | 0.798468866 | 0.481106912 | NTRK1; INSRR | 37 | 1 |
| cg08222513 | 4.89E−09 | 2.08E−03 | −0.317338703 | 0.317338703 | loss | 0.556899409 | 0.239560706 | TRIM31 | 37 | 6 |
| cg11632438 | 7.13E−10 | 3.03E−04 | −0.317308564 | 0.317308564 | loss | 0.467945623 | 0.150637059 | MFSD1; MFSD1 | 37 | 3 |
| cg17369032 | 7.13E−10 | 3.03E−04 | −0.317219004 | 0.317219004 | loss | 0.735280092 | 0.418061088 | NGFR; LOC100288866 | 37 | 17 |
| cg19983801 | 1.01E−09 | 4.27E−04 | −0.317206894 | 0.317206894 | loss | 0.805970258 | 0.488763365 | MUC4; MUC4; MUC4; MUC4; | 37 | 3 |
| | | | | | | | | MUC4; MUC4 | | |
| cg14843888 | 7.13E−10 | 3.03E−04 | −0.317126018 | 0.317126018 | loss | 0.721322642 | 0.404196624 | CACNA1D; CACNA1D; CACNA1D | 37 | 3 |
| cg04502620 | 7.13E−10 | 3.03E−04 | −0.31709224 | 0.31709224 | loss | 0.736934634 | 0.419842394 | DOC2A; DOC2A; DOC2A; DOC2A; | 37 | 16 |
| | | | | | | | | DOC2A; DOC2A | | |
| cg27297505 | 7.13E−10 | 3.03E−04 | −0.317047719 | 0.317047719 | loss | 0.769777066 | 0.452729347 | | 37 | 2 |
| cg01466011 | 7.13E−10 | 3.03E−04 | −0.317037974 | 0.317037974 | loss | 0.684860857 | 0.367822882 | LINC00880 | 37 | 3 |
| cg11498156 | 7.13E−10 | 3.03E−04 | −0.317008749 | 0.317008749 | loss | 0.60365946 | 0.236650712 | TLX1NB; TLX1; TLX1 | 37 | 10 |
| cg07923794 | 7.13E−10 | 3.03E−04 | −0.316946013 | 0.316946013 | loss | 0.909703519 | 0.592757506 | | 37 | 17 |
| cg19362196 | 7.13E−10 | 3.03E−04 | −0.31694212 | 0.31694212 | loss | 0.624076891 | 0.307134771 | STMN1; STMN1; STMN1; STMN1; | 37 | 1 |
| | | | | | | | | MIR3917 | | |
| cg19045731 | 1.10E−09 | 4.65E−04 | −0.316896063 | 0.316896063 | loss | 0.852100751 | 0.535204688 | ESPNL | 37 | 2 |
| cg10225865 | 9.23E−10 | 3.92E−04 | −0.316801099 | 0.316801099 | loss | 0.431812098 | 0.115010999 | | 37 | 8 |
| cg15541170 | 7.13E−10 | 3.03E−04 | −0.316799809 | 0.316799809 | loss | 0.744821179 | 0.428021371 | | 37 | 2 |
| cg16426293 | 5.85E−09 | 2.49E−03 | −0.316691623 | 0.316691623 | loss | 0.52072074 | 0.204029117 | | 37 | 17 |
| cg03243506 | 7.13E−10 | 3.03E−04 | −0.316584799 | 0.316584799 | loss | 0.805984717 | 0.489399918 | TMC2 | 37 | 20 |
| cg08639948 | 7.13E−10 | 3.03E−04 | −0.316565085 | 0.316565085 | loss | 0.697330543 | 0.380765459 | | 37 | 1 |
| cg04534697 | 2.35E−09 | 9.98E−04 | −0.316537217 | 0.316537217 | loss | 0.928770875 | 0.612233659 | | 37 | 6 |
| cg19313311 | 2.35E−09 | 9.98E−04 | −0.316523002 | 0.316523002 | loss | 0.581868508 | 0.265345506 | | 37 | 7 |
| cg16355231 | 1.73E−09 | 7.34E−04 | −0.316457195 | 0.316457195 | loss | 0.393380868 | 0.076923673 | PEX10; PEX10 | 37 | 1 |
| cg11998619 | 7.13E−10 | 3.03E−04 | −0.316391143 | 0.316391143 | loss | 0.723971702 | 0.407580559 | | 37 | 19 |
| cg04164023 | 8.65E−10 | 3.67E−04 | −0.316286286 | 0.316286286 | loss | 0.920739498 | 0.604453212 | PKNOX2 | 37 | 11 |
| cg09713684 | 7.13E−10 | 3.03E−04 | −0.316246819 | 0.316246819 | loss | 0.65450456 | 0.338257741 | C5orf66 | 37 | 5 |
| cg13206556 | 7.13E−10 | 3.03E−04 | −0.316241762 | 0.316241762 | loss | 0.597406015 | 0.281164253 | | 37 | 13 |
| cg04647918 | 1.19E−09 | 5.07E−04 | −0.316219358 | 0.316219358 | loss | 0.691971511 | 0.375752153 | DOC2GP | 37 | 11 |
| cg19894382 | 7.13E−10 | 3.03E−04 | −0.316197664 | 0.316197664 | loss | 0.919666517 | 0.603468853 | | 37 | 10 |
| cg16617366 | 7.13E−10 | 3.03E−04 | −0.316178732 | 0.316178732 | loss | 0.766877874 | 0.450699141 | CTIF; CTIF | 37 | 18 |
| cg25270844 | 7.13E−10 | 3.03E−04 | −0.316162374 | 0.316162374 | loss | 0.702491274 | 0.3863289 | SSC5D; SSC5D | 37 | 19 |
| cg16537676 | 7.13E−10 | 3.03E−04 | −0.316151118 | 0.316151118 | loss | 0.600211236 | 0.284060118 | DDR1; DDR1; DDR1 | 37 | 6 |
| cg14951955 | 7.13E−10 | 3.03E−04 | −0.316096176 | 0.316096176 | loss | 0.664701617 | 0.348605441 | ATG5; ATG5; ATG5; ATG5; ATG5 | 37 | 6 |
| cg15986919 | 7.13E−10 | 3.03E−04 | −0.315908285 | 0.315908285 | loss | 0.389604713 | 0.073696428 | TMEM190 | 37 | 19 |
| cg00305229 | 7.13E−10 | 3.03E−04 | −0.315852211 | 0.315852211 | loss | 0.850290264 | 0.534438053 | | 37 | 1 |
| cg03929570 | 7.13E−10 | 3.03E−04 | −0.315832882 | 0.315832882 | loss | 0.619132764 | 0.303299882 | | 37 | 16 |
| cg02788857 | 7.13E−10 | 3.03E−04 | −0.315786848 | 0.315786848 | loss | 0.857130589 | 0.541343741 | PIWIL2; PIWIL2 | 37 | 8 |
| cg01362541 | 7.13E−10 | 3.03E−04 | −0.315779926 | 0.315779926 | loss | 0.624742296 | 0.308962371 | BMP7 | 37 | 20 |
| cg24435209 | 3.02E−09 | 1.28E−03 | −0.315768731 | 0.315768731 | loss | 0.567429055 | 0.251660324 | CECR5; CECR5; CECR5- | 37 | 22 |
| | | | | | | | | AS1; CECR5-AS1 | | |
| cg16748643 | 7.77E−10 | 3.30E−04 | −0.315647905 | 0.315647905 | loss | 0.64435427 | 0.328706365 | | 37 | 16 |
| cg13561807 | 7.13E−10 | 3.03E−04 | −0.315616108 | 0.315616108 | loss | 0.756557843 | 0.440941735 | LOXHD1 | 37 | 18 |
| cg24258529 | 1.68E−09 | 7.12E−04 | −0.315613741 | 0.315613741 | loss | 0.800491764 | 0.484878024 | | 37 | 4 |
| cg22695379 | 7.13E−10 | 3.03E−04 | −0.315592986 | 0.315592986 | loss | 0.883828368 | 0.568235382 | GFRA3 | 37 | 5 |
| cg01677628 | 1.68E−09 | 7.12E−04 | −0.315219671 | 0.315219671 | loss | 0.645247677 | 0.330028006 | | 37 | 11 |
| cg04487205 | 7.77E−10 | 3.30E−04 | −0.31514487 | 0.31514487 | loss | 0.518934623 | 0.203789753 | C17orf98; C17orf98 | 37 | 17 |
| cg04915618 | 3.02E−09 | 1.28E−03 | −0.315123882 | 0.315123882 | loss | 0.6166052 | 0.301481318 | KALRN; KALRN | 37 | 3 |
| cg07790079 | 9.23E−10 | 3.92E−04 | −0.31506392 | 0.31506392 | loss | 0.664078696 | 0.349014776 | | 37 | 1 |
| cg21911276 | 7.13E−10 | 3.03E−04 | −0.315018379 | 0.315018379 | loss | 0.45402826 | 0.139009881 | NARS | 37 | 18 |
| cg23820816 | 1.42E−09 | 6.01E−04 | −0.314949413 | 0.314949413 | loss | 0.595753208 | 0.280803794 | TRIM2; TRIM2 | 37 | 4 |
| cg26036375 | 1.01E−09 | 4.27E−04 | −0.314906042 | 0.314906042 | loss | 0.889597172 | 0.574691129 | | 37 | 2 |
| cg02661079 | 7.13E−10 | 3.03E−04 | −0.314869807 | 0.314869807 | loss | 0.392265581 | 0.077395774 | CDH22 | 37 | 20 |
| cg20418125 | 7.13E−10 | 3.03E−04 | −0.314730018 | 0.314730018 | loss | 0.841383042 | 0.526653024 | OPCML | 37 | 11 |
| cg23146346 | 7.13E−10 | 3.03E−04 | −0.314604257 | 0.314604257 | loss | 0.759897592 | 0.445293335 | RBM24; RBM24; RBM24 | 37 | 6 |
| cg26590664 | 7.77E−10 | 3.30E−04 | 0.314493618 | 0.314493618 | gain | 0.135228494 | 0.449722112 | ZNF833P | 37 | 19 |
| cg19431051 | 8.47E−10 | 3.60E−04 | −0.314459466 | 0.314459466 | loss | 0.799599408 | 0.485139941 | LINC00301 | 37 | 11 |
| cg02283735 | 1.83E−09 | 7.75E−04 | −0.314415826 | 0.314415826 | loss | 0.633282617 | 0.318866791 | PTPRF; PTPRF | 37 | 1 |
| cg17158564 | 6.35E−09 | 2.70E−03 | −0.314407504 | 0.314407504 | loss | 0.803537609 | 0.489130106 | UCN3 | 37 | 10 |
| cg05936300 | 7.13E−10 | 3.03E−04 | −0.314398863 | 0.314398863 | loss | 0.678894381 | 0.364495518 | TP73; TP73; TP73; TP73; TP73; | 37 | 1 |
| | | | | | | | | TP73 | | |
| cg25288155 | 7.13E−10 | 3.03E−04 | −0.314391053 | 0.314391053 | loss | 0.731189936 | 0.416798882 | PLEKHB1; PLEKHB1; PLEKHB1; | 37 | 11 |
| | | | | | | | | PLEKHB1 | | |
| cg23079252 | 1.96E−08 | 8.33E−03 | −0.314385043 | 0.314385043 | loss | 0.512642149 | 0.198257106 | | 37 | 6 |
| cg27583138 | 9.23E−10 | 3.92E−04 | −0.314345764 | 0.314345764 | loss | 0.526104275 | 0.211758512 | GMNN; GMNN; GMNN; GMNN | 37 | 6 |
| cg05393297 | 6.35E−09 | 2.70E−03 | −0.31433865 | 0.31433865 | loss | 0.530887374 | 0.216548724 | | 37 | 12 |
| cg03710889 | 7.13E−10 | 3.03E−04 | −0.314310849 | 0.314310849 | loss | 0.530845791 | 0.216534941 | SLC11A1 | 37 | 2 |
| cg16080552 | 2.91E−08 | 1.23E−02 | −0.314278497 | 0.314278497 | loss | 0.493442419 | 0.179163922 | TACSTD2; TACSTD2 | 37 | 1 |
| cg04217177 | 7.77E−10 | 3.30E−04 | −0.314238411 | 0.314238411 | loss | 0.658295058 | 0.344056647 | ESRRG | 37 | 1 |
| cg06620514 | 7.13E−10 | 3.03E−04 | −0.314237616 | 0.314237616 | loss | 0.564364351 | 0.250126735 | | 37 | 14 |
| cg17878151 | 3.28E−09 | 1.39E−03 | −0.314235487 | 0.314235487 | loss | 0.686280592 | 0.372045106 | | 37 | 15 |
| cg00892368 | 8.47E−10 | 3.60E−04 | −0.314213882 | 0.314213882 | loss | 0.667320447 | 0.353106565 | | 37 | 6 |
| cg23863328 | 7.13E−10 | 3.03E−04 | −0.314167009 | 0.314167009 | loss | 0.785391579 | 0.471224571 | | 37 | 10 |
| cg22805485 | 2.16E−09 | 9.17E−04 | −0.314119674 | 0.314119674 | loss | 0.892433015 | 0.578313341 | SPON1 | 37 | 11 |
| cg26976412 | 9.23E−10 | 3.92E−04 | −0.314097101 | 0.314097101 | loss | 0.434551617 | 0.120454516 | RAI1 | 37 | 17 |
| cg00646396 | 7.13E−10 | 3.03E−04 | −0.314080906 | 0.314080906 | loss | 0.498920047 | 0.184839141 | | 37 | 1 |
| cg04888360 | 1.01E−09 | 4.27E−04 | −0.314062202 | 0.314062202 | loss | 0.708579591 | 0.394517388 | CRTAC1; CRTAC1 | 37 | 10 |
| cg15901783 | 7.13E−10 | 3.03E−04 | −0.3140472 | 0.3140472 | loss | 0.815029342 | 0.500982141 | KCTD12 | 37 | 13 |
| cg02354563 | 7.13E−10 | 3.03E−04 | −0.314036124 | 0.314036124 | loss | 0.623153647 | 0.309117524 | SLC9A3; SLC9A3 | 37 | 5 |
| cg16092017 | 7.13E−10 | 3.03E−04 | −0.313986386 | 0.313986386 | loss | 0.568359045 | 0.254372659 | | 37 | 5 |
| cg00317879 | 5.39E−09 | 2.29E−03 | −0.313978008 | 0.313978008 | loss | 0.870981396 | 0.557003388 | LOC338694 | 37 | 11 |
| cg02557933 | 7.13E−10 | 3.03E−04 | −0.313977282 | 0.313977282 | loss | 0.653039517 | 0.339062235 | SPEG | 37 | 2 |
| cg26206185 | 7.13E−10 | 3.03E−04 | −0.313896129 | 0.313896129 | loss | 0.859360464 | 0.545464335 | IL17REL; IL17REL | 37 | 22 |
| cg12583970 | 7.13E−10 | 3.03E−04 | −0.313841803 | 0.313841803 | loss | 0.907599062 | 0.593757259 | SFN | 37 | 1 |
| cg03954393 | 7.77E−10 | 3.30E−04 | −0.313831818 | 0.313831818 | loss | 0.833654294 | 0.519822476 | | 37 | 1 |
| cg05756918 | 7.77E−10 | 3.30E−04 | −0.313811589 | 0.313811589 | loss | 0.852215672 | 0.538404082 | | 37 | 7 |
| cg09035529 | 7.13E−10 | 3.03E−04 | −0.313810481 | 0.313810481 | loss | 0.870522445 | 0.556711965 | SLC22A11 | 37 | 11 |
| cg27291468 | 1.68E−09 | 7.12E−04 | −0.313774556 | 0.313774556 | loss | 0.792447668 | 0.478673112 | | 37 | 2 |
| cg02369725 | 4.64E−08 | 1.97E−02 | −0.313734185 | 0.313734185 | loss | 0.724506674 | 0.410772488 | PDE11A; PDE11A | 37 | 2 |
| cg19871388 | 8.47E−10 | 3.60E−04 | −0.313611139 | 0.313611139 | loss | 0.565125957 | 0.251514818 | SALL4; SALL4 | 37 | 20 |
| cg27516568 | 7.77E−10 | 3.30E−04 | −0.313565221 | 0.313565221 | loss | 0.685185904 | 0.371620682 | TMEM155; PP12613 | 37 | 4 |
| cg11537406 | 4.57E−09 | 1.94E−03 | −0.313508923 | 0.313508923 | loss | 0.496334666 | 0.182825744 | SLC37A2; SLC37A2 | 37 | 11 |
| cg12231373 | 5.84E−08 | 2.48E−02 | −0.313420672 | 0.313420672 | loss | 0.900816808 | 0.587396135 | | 37 | 6 |
| cg15743907 | 1.01E−09 | 4.27E−04 | −0.313335354 | 0.313335354 | loss | 0.775795725 | 0.462460371 | PDE4DIP; PDE4DIP; PDE4DIP; | 37 | 1 |
| | | | | | | | | PDE4DIP; PDE4DIP; PDE4DIP; | | |
| | | | | | | | | NBPF9; LOC100288142; | | |
| | | | | | | | | PDE4DIP; PDE4DIP | | |
| cg15193133 | 7.13E−10 | 3.03E−04 | −0.313330257 | 0.313330257 | loss | 0.781379745 | 0.468049488 | | 37 | 17 |
| cg03727673 | 7.13E−10 | 3.03E−04 | −0.313291269 | 0.313291269 | loss | 0.902065128 | 0.588773859 | RAP1GAP; RAP1GAP; RAP1GAP | 37 | 1 |
| cg10816626 | 7.77E−10 | 3.30E−04 | −0.313277339 | 0.313277339 | loss | 0.836678162 | 0.523400824 | CASZ1; CASZ1 | 37 | 1 |
| cg18135555 | 7.13E−10 | 3.03E−04 | −0.313265836 | 0.313265836 | loss | 0.671565325 | 0.358299488 | PIWIL2; PIWIL2 | 37 | 8 |
| cg19370715 | 7.77E−10 | 3.30E−04 | −0.313229412 | 0.313229412 | loss | 0.827022353 | 0.513792941 | CASZ1; CASZ1 | 37 | 1 |
| cg06501366 | 8.47E−10 | 3.60E−04 | −0.313205955 | 0.313205955 | loss | 0.496911355 | 0.1837054 | BHMT2; DMGDH; DMGDH; | 37 | 5 |
| | | | | | | | | BHMT2; DMGDH; DMGDH; | | |
| | | | | | | | | DMGDH; DMGDH | | |
| cg09071762 | 7.13E−10 | 3.03E−04 | −0.313187647 | 0.313187647 | loss | 0.766895842 | 0.453708194 | DPPA5; DPPA5 | 37 | 6 |
| cg01306688 | 1.42E−09 | 6.01E−04 | −0.313181738 | 0.313181738 | loss | 0.857608126 | 0.544426388 | HCAR1; HCAR1 | 37 | 12 |
| cg18322589 | 1.01E−09 | 4.27E−04 | −0.312989202 | 0.312989202 | loss | 0.74668356 | 0.433694359 | TACC2; TACC2 | 37 | 10 |
| cg26090072 | 7.13E−10 | 3.03E−04 | −0.312984567 | 0.312984567 | loss | 0.520817932 | 0.207833365 | RTKN | 37 | 2 |
| cg04876424 | 9.23E−10 | 3.92E−04 | −0.312958184 | 0.312958184 | loss | 0.626823196 | 0.313865012 | RBM24 | 37 | 6 |
| cg04605287 | 3.28E−09 | 1.39E−03 | −0.312949397 | 0.312949397 | loss | 0.573933058 | 0.260983662 | | 37 | 1 |
| cg08109624 | 1.73E−09 | 7.34E−04 | −0.312927934 | 0.312927934 | loss | 0.836371753 | 0.523443819 | | 37 | 1 |
| cg11168104 | 1.54E−08 | 6.56E−03 | −0.312904304 | 0.312904304 | loss | 0.729769204 | 0.4168649 | | 37 | 5 |
| cg14030674 | 7.13E−10 | 3.03E−04 | −0.312892464 | 0.312892464 | loss | 0.811846506 | 0.498954041 | ANK1; ANK1; ANK1; ANK1; | 37 | 8 |
| | | | | | | | | ANK1 | | |
| cg05168015 | 7.77E−10 | 3.30E−04 | −0.312880445 | 0.312880445 | loss | 0.760057309 | 0.447176865 | SH3PXD2B | 37 | 5 |
| cg00256046 | 7.77E−10 | 3.30E−04 | −0.312823402 | 0.312823402 | loss | 0.787006638 | 0.474183235 | ESRRG | 37 | 1 |
| cg12102973 | 7.13E−10 | 3.03E−04 | −0.312736357 | 0.312736357 | loss | 0.860424687 | 0.547688329 | CACNA1A; CACNA1A; CACNA1A; | 37 | 19 |
| | | | | | | | | CACNA1A; CACNA1A | | |
| cg02932167 | 7.13E−10 | 3.03E−04 | −0.312704293 | 0.312704293 | loss | 0.671496281 | 0.358791988 | ECEL1 | 37 | 2 |
| cg08234653 | 7.13E−10 | 3.03E−04 | −0.312699704 | 0.312699704 | loss | 0.595426387 | 0.282726682 | | 37 | 10 |
| cg10347828 | 7.13E−10 | 3.03E−04 | −0.312575556 | 0.312575556 | loss | 0.527349774 | 0.214774218 | RASIP1; RASIP1 | 37 | 19 |
| cg18942934 | 1.01E−09 | 4.27E−04 | −0.312546658 | 0.312546658 | loss | 0.645413628 | 0.332866971 | | 37 | 6 |
| cg02599361 | 1.81E−08 | 7.69E−03 | −0.3125111 | 0.3125111 | loss | 0.605851758 | 0.293340659 | ADAMTS2 | 37 | 5 |
| cg01854076 | 7.13E−10 | 3.03E−04 | −0.312436816 | 0.312436816 | loss | 0.792282492 | 0.479845676 | NATBL | 37 | 4 |
| cg05021771 | 7.13E−10 | 3.03E−04 | −0.312429591 | 0.312429591 | loss | 0.691262138 | 0.378832547 | VAX2 | 37 | 2 |
| cg14882481 | 7.13E−10 | 3.03E−04 | −0.312376499 | 0.312376499 | loss | 0.831146417 | 0.518769918 | ALKBH8 | 37 | 11 |
| cg17830682 | 7.77E−10 | 3.30E−04 | −0.312374367 | 0.312374367 | loss | 0.722704085 | 0.410329718 | LGR6; LGR6; LGR6 | 37 | 1 |
| cg07241675 | 7.13E−10 | 3.03E−04 | −0.312321059 | 0.312321059 | loss | 0.535674547 | 0.223353488 | | 37 | 7 |
| cg08889009 | 7.13E−10 | 3.03E−04 | −0.312305736 | 0.312305736 | loss | 0.359084675 | 0.046778939 | | 37 | 15 |
| cg11218842 | 3.02E−09 | 1.28E−03 | −0.312269391 | 0.312269391 | loss | 0.804858426 | 0.492589035 | UCN3 | 37 | 10 |
| cg02168442 | 7.13E−10 | 3.03E−04 | −0.312254425 | 0.312254425 | loss | 0.492432219 | 0.180177794 | | 37 | 8 |
| cg19766489 | 7.77E−10 | 3.30E−04 | −0.312229715 | 0.312229715 | loss | 0.508370262 | 0.196140547 | GIPC2 | 37 | 1 |
| cg08217910 | 8.47E−10 | 3.60E−04 | −0.312170114 | 0.312170114 | loss | 0.665734938 | 0.353564824 | TMEM37 | 37 | 2 |
| cg24720008 | 1.82E−09 | 7.75E−04 | −0.312152847 | 0.312152847 | loss | 0.582994658 | 0.270841812 | LOC100130539 | 37 | 10 |
| cg02657828 | 7.13E−10 | 3.03E−04 | −0.312144769 | 0.312144769 | loss | 0.816059028 | 0.503914259 | | 37 | 16 |
| cg03446244 | 7.13E−10 | 3.03E−04 | −0.312118747 | 0.312118747 | loss | 0.743596611 | 0.431477865 | LINC01082 | 37 | 16 |
| cg18026225 | 7.13E−10 | 3.03E−04 | −0.312062949 | 0.312062949 | loss | 0.4159679 | 0.103904951 | PLCD3 | 37 | 17 |
| cg01960748 | 3.02E−09 | 1.28E−03 | −0.312010278 | 0.312010278 | loss | 0.842693695 | 0.530683418 | | 37 | 1 |
| cg21136104 | 1.10E−09 | 4.65E−04 | −0.312003395 | 0.312003395 | loss | 0.488567302 | 0.176563906 | | 37 | 3 |
| cg27522367 | 7.13E−10 | 3.03E−04 | −0.31191457 | 0.31191457 | loss | 0.562186953 | 0.250272382 | GPD1; GPD1 | 37 | 12 |
| cg13344095 | 7.13E−10 | 3.03E−04 | −0.311890795 | 0.311890795 | loss | 0.399229536 | 0.087338741 | | 37 | 6 |
| cg23206461 | 8.47E−10 | 3.60E−04 | −0.311864583 | 0.311864583 | loss | 0.64417393 | 0.332309347 | NBPF8; NBPF8; NBPF8 | 37 | 1 |
| cg11704513 | 1.10E−09 | 4.65E−04 | −0.311852527 | 0.311852527 | loss | 0.776605309 | 0.464752782 | COX4I2 | 37 | 20 |
| cg27469738 | 6.35E−09 | 2.70E−03 | −0.311851036 | 0.311851036 | loss | 0.73377233 | 0.421921294 | ANKRD2; ANKRD2 | 37 | 10 |
| cg22243109 | 7.13E−10 | 3.03E−04 | −0.311828268 | 0.311828268 | loss | 0.777796157 | 0.465967888 | | 37 | 17 |
| cg18869061 | 1.19E−09 | 5.07E−04 | −0.311694967 | 0.311694967 | loss | 0.958012885 | 0.646317918 | LYSMD4; LYSMD4; LYSMD4; | 37 | 15 |
| | | | | | | | | LYSMD4; LYSMD4; LYSMD4; | | |
| | | | | | | | | LYSMD4 | | |
| cg13490352 | 7.13E−10 | 3.03E−04 | −0.311669372 | 0.311669372 | loss | 0.476824666 | 0.165155294 | | 37 | 7 |
| cg12588880 | 7.77E−10 | 3.30E−04 | −0.311650647 | 0.311650647 | loss | 0.553680164 | 0.242029518 | | 37 | 2 |
| cg05820241 | 7.13E−10 | 3.03E−04 | −0.311637263 | 0.311637263 | loss | 0.647747204 | 0.336109941 | | 37 | 1 |
| cg04037236 | 7.13E−10 | 3.03E−04 | −0.311625355 | 0.311625355 | loss | 0.826261191 | 0.514635835 | | 37 | 17 |
| cg01601573 | 7.13E−10 | 3.03E−04 | −0.311618297 | 0.311618297 | loss | 0.506220426 | 0.194602129 | GAB1; GAB1 | 37 | 4 |
| cg24762889 | 7.77E−10 | 3.30E−04 | −0.311608464 | 0.311608464 | loss | 0.974274623 | 0.662666159 | | 37 | 6 |
| cg13244241 | 7.13E−10 | 3.03E−04 | −0.31158549 | 0.31158549 | loss | 0.960026466 | 0.648440976 | | 37 | 10 |
| cg13291570 | 1.10E−09 | 4.65E−04 | −0.311569386 | 0.311569386 | loss | 0.47525703 | 0.163587644 | | 37 | 8 |
| cg04473902 | 7.13E−10 | 3.03E−04 | −0.311551952 | 0.311551952 | loss | 0.712624217 | 0.401072265 | PLBD1 | 37 | 12 |
| cg05567511 | 8.47E−10 | 3.60E−04 | −0.311457084 | 0.311457084 | loss | 0.77744796 | 0.465990876 | | 37 | 7 |
| cg05950877 | 1.01E−09 | 4.27E−04 | 0.311432197 | 0.311432197 | gain | 0.101748068 | 0.413180265 | ZNF833P | 37 | 19 |
| cg05239811 | 7.92E−08 | 3.36E−02 | −0.311401772 | 0.311401772 | loss | 0.718163908 | 0.406762135 | VWA7 | 37 | 6 |
| cg06251500 | 7.13E−10 | 3.03E−04 | −0.311398894 | 0.311398894 | loss | 0.876739058 | 0.565340165 | KCNH2; KCNH2 | 37 | 7 |
| cg09517873 | 7.77E−10 | 3.30E−04 | −0.311385334 | 0.311385334 | loss | 0.702221681 | 0.390836347 | DHRS3 | 37 | 1 |
| cg02735058 | 7.13E−10 | 3.03E−04 | −0.311367506 | 0.311367506 | loss | 0.862335794 | 0.550968288 | | 37 | 6 |
| cg16288318 | 3.02E−09 | 1.28E−03 | −0.311295317 | 0.311295317 | loss | 0.66771037 | 0.356415053 | | 37 | 13 |
| cg20518446 | 9.23E−10 | 3.92E−04 | −0.311248066 | 0.311248066 | loss | 0.76616716 | 0.454919094 | AHNAK; AHNAK | 37 | 11 |
| cg13001017 | 7.13E−10 | 3.03E−04 | −0.311198416 | 0.311198416 | loss | 0.846015104 | 0.534816688 | KHDC3L | 37 | 6 |
| cg20236750 | 7.13E−10 | 3.03E−04 | −0.311181672 | 0.311181672 | loss | 0.638454055 | 0.327272382 | LGI1 | 37 | 10 |
| cg08217285 | 7.13E−10 | 3.03E−04 | −0.311178929 | 0.311178929 | loss | 0.432037379 | 0.12085845 | GIT1; GIT1 | 37 | 17 |
| cg24723561 | 9.23E−10 | 3.92E−04 | −0.311172753 | 0.311172753 | loss | 0.593105347 | 0.281932594 | SLC35F3 | 37 | 1 |
| cg08250742 | 7.13E−10 | 3.03E−04 | −0.311161652 | 0.311161652 | loss | 0.867632364 | 0.556470712 | LOC100288866; LOC100288866 | 37 | 17 |
| cg11556846 | 3.57E−09 | 1.52E−03 | −0.311137148 | 0.311137148 | loss | 0.694073672 | 0.382936524 | | 37 | 2 |
| cg03130418 | 7.13E−10 | 3.03E−04 | −0.311070478 | 0.311070478 | loss | 0.814283066 | 0.503212588 | TNXB | 37 | 6 |
| cg08950537 | 7.13E−10 | 3.03E−04 | −0.311001658 | 0.311001658 | loss | 0.55825987 | 0.247258212 | RBP4 | 37 | 10 |
| cg13464393 | 7.13E−10 | 3.03E−04 | −0.3109305 | 0.3109305 | loss | 0.964442994 | 0.653512494 | | 37 | 9 |
| cg14170358 | 7.13E−10 | 3.03E−04 | −0.31087712 | 0.31087712 | loss | 0.550665879 | 0.239788759 | | 37 | 2 |
| cg15597109 | 7.13E−10 | 3.03E−04 | −0.310871745 | 0.310871745 | loss | 0.883545457 | 0.572673712 | CASZ1; CASZ1 | 37 | 1 |
| cg05358404 | 7.13E−10 | 3.03E−04 | −0.3107786 | 0.3107786 | loss | 0.690525406 | 0.379746806 | RTEL1; RTEL1; RTEL1; RTEL1; | 37 | 20 |
| | | | | | | | | RTEL1-TNFRSF6B | | |
| cg04143909 | 1.01E−09 | 4.27E−04 | −0.310629817 | 0.310629817 | loss | 0.774051164 | 0.463421347 | SLC35F3 | 37 | 1 |
| cg05411132 | 2.48E−08 | 1.05E−02 | −0.310563981 | 0.310563981 | loss | 0.780530823 | 0.469966841 | XRCC6P5 | 37 | X |
| cg21489266 | 7.13E−10 | 3.03E−04 | −0.310542328 | 0.310542328 | loss | 0.583386881 | 0.272844553 | TDGF1; TDGF1 | 37 | 3 |
| cg22572476 | 7.13E−10 | 3.03E−04 | −0.31053342 | 0.31053342 | loss | 0.557961538 | 0.247428118 | | 37 | 6 |
| cg20071884 | 8.47E−10 | 3.60E−04 | −0.31051734 | 0.31051734 | loss | 0.82043067 | 0.509913329 | KIAA1210; KIAA1210 | 37 | X |
| cg19910200 | 7.13E−10 | 3.03E−04 | −0.310474102 | 0.310474102 | loss | 0.887567602 | 0.5770935 | TMEM190 | 37 | 19 |
| cg01869233 | 7.13E−10 | 3.03E−04 | −0.310421769 | 0.310421769 | loss | 0.707806228 | 0.397384459 | LRRN4 | 37 | 20 |
| cg14283328 | 7.13E−10 | 3.03E−04 | −0.310357933 | 0.310357933 | loss | 0.541687651 | 0.231329718 | RHOD | 37 | 11 |
| cg18533225 | 7.13E−10 | 3.03E−04 | −0.310261018 | 0.310261018 | loss | 0.495949589 | 0.185688571 | KLHDC7B | 37 | 22 |
| cg07654896 | 7.13E−10 | 3.03E−04 | −0.310148031 | 0.310148031 | loss | 0.649222308 | 0.339074276 | RGN; RGN; RGN; RGN | 37 | X |
| cg03021802 | 7.13E−10 | 3.03E−04 | −0.310140394 | 0.310140394 | loss | 0.470435647 | 0.160295253 | DMTN; DMTN; DMTN; DMTN; | 37 | 8 |
| | | | | | | | | DMTN; DMTN | | |
| cg21391046 | 5.84E−08 | 2.48E−02 | −0.310068274 | 0.310068274 | loss | 0.851966874 | 0.5418986 | | 37 | 4 |
| cg21251018 | 7.13E−10 | 3.03E−04 | −0.310026641 | 0.310026641 | loss | 0.558721317 | 0.248694676 | NKAPL | 37 | 6 |
| cg09906233 | 9.23E−10 | 3.92E−04 | −0.310011724 | 0.310011724 | loss | 0.874605494 | 0.564593771 | ZFYVE28; ZFYVE28; ZFYVE28; | 37 | 4 |
| | | | | | | | | ZFYVE28; ZFYVE28; ZFYVE28 | | |
| cg07476339 | 1.30E−09 | 5.52E−04 | −0.310007231 | 0.310007231 | loss | 0.907943549 | 0.597936318 | | 37 | 7 |
| cg08952407 | 7.13E−10 | 3.03E−04 | −0.309963213 | 0.309963213 | loss | 0.678771972 | 0.368808759 | CCDC152 | 37 | 5 |
| cg23936402 | 1.54E−09 | 6.54E−04 | −0.309832456 | 0.309832456 | loss | 0.625869979 | 0.316037524 | GPX5; GPX5 | 37 | 6 |
| cg02172058 | 7.13E−10 | 3.03E−04 | −0.309807044 | 0.309807044 | loss | 0.672071821 | 0.362264776 | C17orf64 | 37 | 17 |
| cg08812802 | 7.13E−10 | 3.03E−04 | −0.309802928 | 0.309802928 | loss | 0.799381792 | 0.489578865 | | 37 | 12 |
| cg20432211 | 7.13E−10 | 3.03E−04 | −0.309782873 | 0.309782873 | loss | 0.452616381 | 0.142833508 | | 37 | 4 |
| cg04731038 | 1.10E−09 | 4.65E−04 | −0.309749346 | 0.309749346 | loss | 0.57690447 | 0.267155124 | | 37 | 17 |
| cg21716282 | 7.13E−10 | 3.03E−04 | −0.309707996 | 0.309707996 | loss | 0.616412502 | 0.306704506 | | 37 | 3 |
| cg15769343 | 7.13E−10 | 3.03E−04 | −0.309673911 | 0.309673911 | loss | 0.595536428 | 0.285862518 | | 37 | 12 |
| cg07207789 | 7.13E−10 | 3.03E−04 | −0.309663318 | 0.309663318 | loss | 0.7198836 | 0.410220282 | CRISPLD2 | 37 | 16 |
| cg24263062 | 9.22E−08 | 3.91E−02 | −0.309618082 | 0.309618082 | loss | 0.581332453 | 0.271714371 | EBF4 | 37 | 20 |
| cg13029400 | 7.13E−10 | 3.03E−04 | −0.309616331 | 0.309616331 | loss | 0.813406166 | 0.503789835 | ZBTB38 | 37 | 3 |
| cg02834449 | 7.77E−10 | 3.30E−04 | −0.309610095 | 0.309610095 | loss | 0.694201419 | 0.384591324 | C16orf74 | 37 | 16 |
| cg03124514 | 7.13E−10 | 3.03E−04 | −0.309595974 | 0.309595974 | loss | 0.910594704 | 0.600998729 | | 37 | 5 |
| cg17519696 | 7.13E−10 | 3.03E−04 | −0.30957129 | 0.30957129 | loss | 0.847966355 | 0.538395065 | OCA2 | 37 | 15 |
| cg25985355 | 3.28E−09 | 1.39E−03 | −0.309558318 | 0.309558318 | loss | 0.473298157 | 0.163739839 | | 37 | 7 |
| cg10043155 | 7.13E−10 | 3.03E−04 | −0.309453161 | 0.305453161 | loss | 0.950449502 | 0.640996341 | | 37 | 6 |
| cg16595288 | 7.13E−10 | 3.03E−04 | −0.309192937 | 0.309192937 | loss | 0.616385249 | 0.307192312 | RASGEF1A; RASGEF1A | 37 | 10 |
| cg00737841 | 1.54E−08 | 6.56E−03 | −0.309141804 | 0.309141804 | loss | 0.760423204 | 0.4512814 | | 37 | 10 |
| cg26687119 | 7.13E−10 | 3.03E−04 | −0.309094503 | 0.309094503 | loss | 0.644244515 | 0.335150012 | NTRK1; INSRR | 37 | 1 |
| cg15638292 | 7.13E−10 | 3.03E−04 | −0.309094051 | 0.309094051 | loss | 0.587037887 | 0.277943835 | MYO7A; MYO7A; MYO7A | 37 | 11 |
| cg27196974 | 1.54E−09 | 6.54E−04 | −0.309063665 | 0.309063655 | loss | 0.595716289 | 0.286652624 | | 37 | 17 |
| cg01306824 | 7.84E−10 | 3.33E−04 | −0.308978281 | 0.308978251 | loss | 0.909637752 | 0.600659471 | | 37 | 17 |
| cg23105130 | 7.84E−10 | 3.33E−04 | −0.30893666 | 0.30893666 | loss | 0.555594242 | 0.246657582 | MAPK15 | 37 | 8 |
| cg07726287 | 7.13E−10 | 3.03E−04 | −0.308898328 | 0.308898328 | loss | 0.577025287 | 0.268126959 | | 37 | 16 |
| cg14759277 | 7.13E−10 | 3.03E−04 | −0.308814928 | 0.308814928 | loss | 0.932693145 | 0.623878218 | CACNA1G; CACNA1G; CACNA1G; | 37 | 17 |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G | | |
| cg05134926 | 2.12E−08 | 9.01E−03 | −0.308790448 | 0.308790448 | loss | 0.696447819 | 0.387657371 | KLHL14 | 37 | 18 |
| cg12161935 | 7.13E−10 | 3.03E−04 | −0.308690764 | 0.308690764 | loss | 0.866007264 | 0.5573165 | | 37 | 2 |
| cg08134856 | 7.13E−10 | 3.03E−04 | −0.30852562 | 0.30852562 | loss | 0.576898249 | 0.268372629 | STMN1; STMN1; STMN1; STMN1; | 37 | 1 |
| | | | | | | | | MIR3917 | | |
| cg17870997 | 6.80E−08 | 2.89E−02 | −0.308521714 | 0.308521714 | loss | 0.706180685 | 0.397658971 | | 37 | 2 |
| cg04169774 | 7.13E−10 | 3.03E−04 | −0.308457037 | 0.308457037 | loss | 0.73927636 | 0.430819324 | PER2 | 37 | 2 |
| cg13404038 | 7.13E−10 | 3.03E−04 | −0.308448631 | 0.308448631 | loss | 0.666050649 | 0.357602018 | CRB2 | 37 | 9 |
| cg27357306 | 7.13E−10 | 3.03E−04 | −0.308418109 | 0.308418109 | loss | 0.416020983 | 0.107602874 | | 37 | 10 |
| cg21434046 | 7.13E−10 | 3.03E−04 | −0.308339386 | 0.308339386 | loss | 0.738397798 | 0.430058412 | | 37 | 18 |
| cg01161644 | 7.13E−10 | 3.03E−04 | −0.308324469 | 0.308324469 | loss | 0.703327045 | 0.395002576 | | 37 | 16 |
| cg12432038 | 7.13E−10 | 3.03E−04 | −0.308304471 | 0.308304471 | loss | 0.6800409 | 0.371736429 | C1orf229; C1orf229 | 37 | 1 |
| cg19628964 | 7.13E−10 | 3.03E−04 | −0.308254242 | 0.308254242 | loss | 0.751434089 | 0.443179847 | LOC401242 | 37 | 6 |
| cg02462416 | 7.84E−10 | 3.33E−04 | −0.308207464 | 0.308207464 | loss | 0.582458717 | 0.274251253 | | 37 | 11 |
| cg03696370 | 7.77E−10 | 3.30E−04 | −0.308172499 | 0.308172499 | loss | 0.888256387 | 0.580083888 | PEBP4 | 37 | 8 |
| cg02784823 | 1.67E−08 | 7.10E−03 | −0.308111859 | 0.30811859 | loss | 0.712922913 | 0.404811054 | LMTK3 | 37 | 19 |
| cg21936959 | 7.13E−10 | 3.03E−04 | −0.308023408 | 0.308023408 | loss | 0.921184149 | 0.613160741 | MRGPRF; MRGPRF | 37 | 11 |
| cg15770575 | 1.19E−09 | 5.07E−04 | −0.307821675 | 0.307821675 | loss | 0.683575028 | 0.375753353 | | 37 | 22 |
| cg07991586 | 7.13E−10 | 3.03E−04 | −0.307789095 | 0.307789095 | loss | 0.85403663 | 0.546247535 | | 37 | 16 |
| cg07455790 | 2.48E−08 | 1.05E−02 | −0.307700737 | 0.307700737 | loss | 0.678077719 | 0.370376982 | | 37 | 6 |
| cg03671431 | 7.13E−10 | 3.03E−04 | −0.307682272 | 0.307682272 | loss | 0.638057672 | 0.3304054 | | 37 | 18 |
| cg21151061 | 7.13E−10 | 3.03E−04 | −0.307671535 | 0.307671535 | loss | 0.749356058 | 0.441684524 | GPD1; GPD1 | 37 | 12 |
| cg14476334 | 7.13E−10 | 3.03E−04 | −0.307653831 | 0.307653831 | loss | 0.705276502 | 0.397622671 | | 37 | 1 |
| cg27237745 | 7.84E−10 | 3.33E−04 | −0.307581964 | 0.307581964 | loss | 0.910115452 | 0.602533488 | KHDC3L | 37 | 6 |
| cg09101902 | 7.13E−10 | 3.03E−04 | −0.307574112 | 0.307574112 | loss | 0.471997347 | 0.164423235 | ARHGAP22 | 37 | 10 |
| cg21597025 | 1.01E−09 | 4.27E−04 | −0.307457894 | 0.307457894 | loss | 0.782972494 | 0.4755146 | CHST2 | 37 | 3 |
| cg07589381 | 7.77E−10 | 3.30E−04 | −0.307448257 | 0.307448257 | loss | 0.697358398 | 0.389910141 | CASZ1; CASZ1 | 37 | 1 |
| cg11823142 | 8.80E−09 | 3.74E−03 | −0.307425641 | 0.307425641 | loss | 0.843672606 | 0.536246965 | | 37 | 6 |
| cg12130768 | 7.13E−10 | 3.03E−04 | −0.307425038 | 0.307425038 | loss | 0.511318279 | 0.203893241 | USP43; USP43 | 37 | 17 |
| cg27206976 | 7.13E−10 | 3.03E−04 | −0.307398387 | 0.307398387 | loss | 0.51089877 | 0.203500382 | PLXNB2 | 37 | 22 |
| cg22496723 | 7.13E−10 | 3.03E−04 | −0.307386054 | 0.307386054 | loss | 0.870224513 | 0.562838459 | CACNG2 | 37 | 22 |
| cg20031364 | 7.13E−10 | 3.03E−04 | −0.307355149 | 0.307355149 | loss | 0.537017619 | 0.2296625 | SRCIN1 | 37 | 17 |
| cg14457311 | 7.13E−10 | 3.03E−04 | −0.307279613 | 0.307279613 | loss | 0.733933308 | 0.426653694 | CACNA1G; CACNA1G; CACNA1G; | 37 | 17 |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G | | |
| cg13211302 | 7.13E−10 | 3.03E−04 | −0.30724426 | 0.30724426 | loss | 0.530725913 | 0.223481653 | | 37 | 8 |
| cg11348165 | 7.13E−10 | 3.03E−04 | −0.307228329 | 0.307228329 | loss | 0.650605864 | 0.343377535 | SFN | 37 | 1 |
| cg19545472 | 7.13E−10 | 3.03E−04 | −0.307212314 | 0.307212314 | loss | 0.682853443 | 0.375541129 | GFRA4; GFRA4; GFRA4; GFRA4 | 37 | 20 |
| cg05056638 | 7.48E−09 | 3.18E−03 | −0.307193291 | 0.307193291 | loss | 0.637872379 | 0.330679088 | | 37 | 8 |
| cg12207153 | 1.83E−09 | 7.75E−04 | −0.307173917 | 0.307173917 | loss | 0.649644428 | 0.342470512 | | 37 | 1 |
| cg16351246 | 1.01E−09 | 4.27E−04 | −0.307154709 | 0.307154709 | loss | 0.497178972 | 0.190024262 | | 37 | 13 |
| cg13053129 | 7.13E−10 | 3.03E−04 | −0.307077915 | 0.307077915 | loss | 0.865150009 | 0.558072094 | | 37 | 19 |
| cg27503360 | 7.13E−10 | 3.03E−04 | −0.307060193 | 0.307060193 | loss | 0.584766228 | 0.277706035 | CRHR1; CRHR1; CRHR1; CRHR1; | 37 | 17 |
| | | | | | | | | CRHR1 | | |
| cg09552892 | 7.13E−10 | 3.03E−04 | −0.30704779 | 0.30704779 | loss | 0.533492996 | 0.226445206 | SNCG; MMRN2 | 37 | 10 |
| cg24049888 | 7.13E−10 | 3.03E−04 | −0.307002845 | 0.307002845 | loss | 0.327952862 | 0.020950017 | POU2AF1; POU2AF1 | 37 | 11 |
| cg21461564 | 9.23E−10 | 3.92E−04 | −0.306998092 | 0.306998092 | loss | 0.525869662 | 0.218871571 | TTPA | 37 | 8 |
| cg03661817 | 1.10E−09 | 4.65E−04 | −0.306941924 | 0.306941924 | loss | 0.793956177 | 0.487014253 | KIFC3; KIFC3; KIFC3; KIFC3 | 37 | 16 |
| cg00847857 | 2.16E−09 | 9.17E−04 | −0.306857586 | 0.306857586 | loss | 0.723513504 | 0.416655918 | LINC00189 | 37 | 21 |
| cg02141929 | 1.10E−09 | 4.65E−04 | −0.306628883 | 0.306628883 | loss | 0.65557503 | 0.348946147 | | 37 | 16 |
| cg09817162 | 3.02E−09 | 1.28E−03 | −0.30662414 | 0.30662414 | loss | 0.794639834 | 0.488015694 | | 37 | 6 |
| cg26182859 | 8.47E−10 | 3.60E−04 | −0.306608241 | 0.306608241 | loss | 0.458668587 | 0.152060345 | | 37 | 1 |
| cg27179866 | 7.13E−10 | 3.03E−04 | −0.306578869 | 0.306578869 | loss | 0.492544481 | 0.185965612 | | 37 | 6 |
| cg25341560 | 7.13E−10 | 3.03E−04 | −0.306577281 | 0.306577281 | loss | 0.506183481 | 0.1996062 | SCN1B; HPN; HPN | 37 | 19 |
| cg09765983 | 7.34E−08 | 3.12E−02 | −0.306495868 | 0.306495868 | loss | 0.684303626 | 0.377807759 | | 37 | 4 |
| cg00274965 | 7.13E−10 | 3.03E−04 | −0.306481993 | 0.306481993 | loss | 0.340716387 | 0.034234394 | | 37 | 21 |
| cg03115048 | 1.19E−09 | 5.07E−04 | −0.306396508 | 0.306396508 | loss | 0.818269649 | 0.511873141 | | 37 | 1 |
| cg15534366 | 7.13E−10 | 3.03E−04 | −0.306383212 | 0.306383212 | loss | 0.793504094 | 0.487120882 | CDH4 | 37 | 20 |
| cg13125537 | 7.13E−10 | 3.03E−04 | −0.306374598 | 0.306374598 | loss | 0.759746592 | 0.453371994 | | 37 | 17 |
| cg06314987 | 7.13E−10 | 3.03E−04 | −0.306347913 | 0.306347913 | loss | 0.854837519 | 0.558489606 | FOXL1 | 37 | 16 |
| cg25604594 | 5.85E−09 | 2.49E−03 | −0.306309385 | 0.306309385 | loss | 0.746364926 | 0.440055541 | ANKRD2; ANKRD2 | 37 | 10 |
| cg26477792 | 2.16E−09 | 9.17E−04 | −0.30624528 | 0.30624528 | loss | 0.509845826 | 0.203600546 | | 37 | 6 |
| cg23467079 | 1.54E−08 | 6.56E−03 | −0.306241084 | 0.306241084 | loss | 0.699226919 | 0.392985835 | FAM83A; FAM83A | 37 | 8 |
| cg00592643 | 3.02E−09 | 1.28E−03 | −0.306207738 | 0.306207738 | loss | 0.738563191 | 0.432355453 | BTBD3; BTBD3 | 37 | 20 |
| cg01862363 | 7.77E−10 | 3.30E−04 | −0.306164818 | 0.306164818 | loss | 0.451819338 | 0.14565452 | NTN1 | 37 | 17 |
| cg03879180 | 7.13E−10 | 3.03E−04 | −0.306159251 | 0.306159251 | loss | 0.652688804 | 0.346529553 | | 37 | 2 |
| cg13744039 | 7.13E−10 | 3.03E−04 | −0.30609155 | 0.30609155 | loss | 0.753996338 | 0.447904788 | | 37 | 3 |
| cg24261333 | 7.13E−10 | 3.03E−04 | −0.306084881 | 0.306084881 | loss | 0.518802375 | 0.212717494 | PLEKHB1; PLEKHB1 | 37 | 11 |
| cg23335299 | 7.13E−10 | 3.03E−04 | −0.305982798 | 0.305982798 | loss | 0.575967781 | 0.272984983 | | 37 | 15 |
| cg18376352 | 7.13E−10 | 3.03E−04 | −0.305976721 | 0.305976721 | loss | 0.619913609 | 0.313936888 | CRLF1 | 37 | 19 |
| cg01914059 | 7.13E−10 | 3.03E−04 | −0.305962887 | 0.305962887 | loss | 0.739579375 | 0.433616438 | | 37 | 16 |
| cg10958002 | 7.13E−10 | 3.03E−04 | −0.305935138 | 0.305935138 | loss | 0.426491832 | 0.120556694 | SYN2; TIMP4; SYN2 | 37 | 3 |
| cg14785609 | 7.13E−10 | 3.03E−04 | −0.305902198 | 0.305902198 | loss | 0.409402785 | 0.103500586 | | 37 | 1 |
| cg17962638 | 7.77E−10 | 3.30E−04 | −0.305872403 | 0.305872403 | loss | 0.473574517 | 0.167702114 | | 37 | 6 |
| cg26535593 | 8.47E−10 | 3.60E−04 | −0.305866787 | 0.305866787 | loss | 0.468362434 | 0.162495647 | ZNF670; ZNF670; ZNF670- | 37 | 1 |
| | | | | | | | | ZNF695 | | |
| cg08745665 | 7.13E−10 | 3.03E−04 | −0.305827413 | 0.305827413 | loss | 0.844759025 | 0.538931612 | ZNF518B | 37 | 4 |
| cg09506575 | 1.19E−09 | 5.07E−04 | −0.305736599 | 0.305736599 | loss | 0.516157902 | 0.210421303 | GPR85; GPR85; GPR85; GPR85; | 37 | 7 |
| | | | | | | | | GPR85 | | |
| cg20941258 | 7.13E−10 | 3.03E−04 | −0.305719726 | 0.305719726 | loss | 0.518214391 | 0.212494665 | TDGF1; TDGF1 | 37 | 3 |
| cg06891815 | 7.13E−10 | 3.03E−04 | −0.305659194 | 0.305659194 | loss | 0.7564866 | 0.450827406 | NOTCH4 | 37 | 6 |
| cg21062347 | 7.13E−10 | 3.03E−04 | −0.305657437 | 0.305657437 | loss | 0.560731296 | 0.255073859 | SLC22A8; SLC22A8; SLC22A8; | 37 | 11 |
| | | | | | | | | SLC22A8 | | |
| cg08260245 | 4.21E−09 | 1.79E−03 | −0.30565608 | 0.30565608 | loss | 0.651913898 | 0.346257818 | CPT1B; CPT1B; CPT1B; CPT1B; | 37 | 22 |
| | | | | | | | | CPT1B; CPT1B; CPT1B; | | |
| | | | | | | | | CPT1B; CHKB-CPT1B | | |
| cg21700780 | 7.77E−10 | 3.30E−04 | −0.305649264 | 0.305649264 | loss | 0.839507864 | 0.5338586 | MYOM2 | 37 | 8 |
| cg25792518 | 1.30E−09 | 5.52E−04 | −0.305459066 | 0.305459066 | loss | 0.686338419 | 0.380879353 | CHAD; ACSF2 | 37 | 17 |
| cg08169827 | 7.13E−10 | 3.03E−04 | −0.30538293 | 0.30538293 | loss | 0.432039417 | 0.126656487 | | 37 | 1 |
| cg11715966 | 7.13E−10 | 3.03E−04 | −0.305381925 | 0.305381925 | loss | 0.885710166 | 0.580328241 | CCDC8 | 37 | 19 |
| cg01733570 | 7.13E−10 | 3.03E−04 | −0.305290861 | 0.305290861 | loss | 0.706297032 | 0.401006171 | DFNA5; DFNA5; DFNA5; DFNA5; | 37 | 7 |
| | | | | | | | | DFNA5 | | |
| cg12758973 | 1.19E−09 | 5.07E−04 | −0.305282448 | 0.305282448 | loss | 0.637585389 | 0.332302941 | GCSAML; GCSAML; GCSAML; | 37 | 1 |
| | | | | | | | | GCSAML; GCSAML; GCSAML; | | |
| | | | | | | | | OR2C3; GCSAML- | | |
| | | | | | | | | AS1; GCSAML-AS1 | | |
| cg21051964 | 7.13E−10 | 3.03E−04 | −0.30526735 | 0.30526735 | loss | 0.828494626 | 0.523227276 | B4GALNT1; B4GALNT1 | 37 | 12 |
| cg12499235 | 1.83E−09 | 7.75E−04 | −0.305230026 | 0.305230026 | loss | 0.838177796 | 0.532947771 | ASCL2 | 37 | 11 |
| cg04149978 | 7.13E−10 | 3.03E−04 | −0.305176834 | 0.305176894 | loss | 0.778964964 | 0.473788071 | CASZ1; CASZ1 | 37 | 1 |
| cg09795979 | 7.13E−10 | 3.03E−04 | −0.305070877 | 0.305070877 | loss | 0.817728394 | 0.512657518 | SYT13; SYT13 | 37 | 11 |
| cg06260709 | 7.77E−10 | 3.30E−04 | −0.305044847 | 0.305044847 | loss | 0.510171194 | 0.205126347 | | 37 | 20 |
| cg03456771 | 6.80E−08 | 2.89E−02 | −0.3049531 | 0.3049531 | loss | 0.755161553 | 0.450208453 | | 37 | 7 |
| cg18062205 | 7.13E−10 | 3.03E−04 | −0.304947862 | 0.304947862 | loss | 0.617362462 | 0.3124146 | KCNG1 | 37 | 20 |
| cg00095526 | 7.13E−10 | 3.03E−04 | −0.304859699 | 0.304859699 | loss | 0.780320828 | 0.475461129 | CACNA1S; CACNA1S | 37 | 1 |
| cg00944166 | 7.77E−10 | 3.30E−04 | −0.304791556 | 0.304791556 | loss | 0.970691015 | 0.665899459 | SLCO6A1; SLCO6A1 | 37 | 5 |
| cg19872936 | 7.13E−10 | 3.03E−04 | −0.304770683 | 0.304770683 | loss | 0.723871989 | 0.419101306 | ESRRB | 37 | 14 |
| cg14320626 | 7.77E−10 | 3.30E−04 | −0.304701257 | 0.304701257 | loss | 0.708529198 | 0.403827941 | | 37 | 4 |
| cg26014401 | 7.13E−10 | 3.03E−04 | −0.304636283 | 0.304636283 | loss | 0.533236336 | 0.228600053 | | 37 | 22 |
| cg23823894 | 7.13E−10 | 3.03E−04 | −0.304584339 | 0.304584339 | loss | 0.847942557 | 0.543358218 | | 37 | 8 |
| cg14724749 | 7.13E−10 | 3.03E−04 | −0.30451471 | 0.30451471 | loss | 0.68197304 | 0.377458329 | | 37 | 19 |
| cg03547924 | 7.77E−10 | 3.30E−04 | −0.304506411 | 0.304506411 | loss | 0.823734417 | 0.519228006 | GDF5; GDF5 | 37 | 20 |
| cg27147718 | 7.77E−10 | 3.30E−04 | −0.304477201 | 0.304477201 | loss | 0.821838283 | 0.517361082 | NDRG4; NDRG4 | 37 | 16 |
| cg00108349 | 7.13E−10 | 3.03E−04 | −0.304459145 | 0.304459145 | loss | 0.767977398 | 0.463518253 | | 37 | 13 |
| cg27454064 | 9.23E−10 | 3.92E−04 | −0.304457494 | 0.304457494 | loss | 0.422953842 | 0.118496347 | | 37 | 12 |
| cg14892768 | 7.13E−10 | 3.03E−04 | −0.304451762 | 0.304451762 | loss | 0.524872621 | 0.220420859 | AXL; AXL | 37 | 19 |
| cg11213150 | 7.13E−10 | 3.03E−04 | −0.304397106 | 0.304397106 | loss | 0.662204353 | 0.357807247 | RALGPS1; RALGPS1; RALGPS1; | 37 | 9 |
| | | | | | | | | ANGPTL2; RALGPS1 | | |
| cg20951444 | 7.13E−10 | 3.03E−04 | −0.304379866 | 0.304379866 | loss | 0.734135572 | 0.429755706 | FNDC5; FNDC5; FNDC5 | 37 | 1 |
| cg12602851 | 1.22E−08 | 5.16E−03 | −0.304365801 | 0.304365801 | loss | 0.641322583 | 0.336956782 | IMPG1; IMPG1 | 37 | 6 |
| cg04079760 | 7.77E−10 | 3.30E−04 | −0.304243547 | 0.304243547 | loss | 0.411573281 | 0.107329734 | ESRP2 | 37 | 16 |
| cg10036920 | 8.47E−10 | 3.60E−04 | −0.304214648 | 0.304214648 | loss | 0.662338519 | 0.358123871 | LRCOL1 | 37 | 12 |
| cg26608305 | 7.13E−10 | 3.03E−04 | −0.304210876 | 0.304210876 | loss | 0.612633853 | 0.308422976 | AP2A2; AP2A2 | 37 | 11 |
| cg26516532 | 7.13E−10 | 3.03E−04 | −0.304158493 | 0.304158493 | loss | 0.508270534 | 0.204112041 | | 37 | 1 |
| cg13872005 | 7.13E−10 | 3.03E−04 | −0.30408635 | 0.30408635 | loss | 0.352421717 | 0.048335367 | | 37 | 2 |
| cg02049405 | 1.19E−09 | 5.07E−04 | −0.303995114 | 0.303995114 | loss | 0.544024991 | 0.240029876 | | 37 | 6 |
| cg03622666 | 2.16E−09 | 9.17E−04 | −0.303979352 | 0.303979352 | loss | 0.62569707 | 0.321717718 | | 37 | 16 |
| cg21950166 | 7.13E−10 | 3.03E−04 | −0.303955623 | 0.303955623 | loss | 0.629768875 | 0.325813253 | SFN | 37 | 1 |
| cg19250304 | 7.13E−10 | 3.03E−04 | −0.303955601 | 0.303955601 | loss | 0.570529772 | 0.266574171 | PITRM1; PITRM1; PITRM1 | 37 | 10 |
| cg09163477 | 7.13E−10 | 3.03E−04 | −0.303948561 | 0.303948561 | loss | 0.788399938 | 0.484451376 | | 37 | 12 |
| cg18706028 | 8.47E−10 | 3.60E−04 | −0.303934221 | 0.303934221 | loss | 0.624948226 | 0.321014006 | CCKBR | 37 | 11 |
| cg19340941 | 1.01E−09 | 4.27E−04 | −0.303909075 | 0.303909075 | loss | 0.663423987 | 0.359514912 | RBM46; RBM46; RBM46 | 37 | 4 |
| cg17745392 | 7.77E−10 | 3.30E−04 | −0.303891107 | 0.303891107 | loss | 0.806778177 | 0.502887071 | LRRC4B | 37 | 19 |
| cg18092474 | 1.87E−09 | 7.93E−04 | −0.303885412 | 0.303885412 | loss | 0.541304512 | 0.2374191 | CYP1A1 | 37 | 15 |
| cg07740525 | 7.13E−10 | 3.03E−04 | −0.303835239 | 0.303835239 | loss | 0.653752898 | 0.349917659 | KIF12; KIF12 | 37 | 9 |
| cg08922729 | 7.77E−10 | 3.30E−04 | −0.303796416 | 0.303796416 | loss | 0.593766551 | 0.289970135 | | 37 | 1 |
| cg11885357 | 3.69E−09 | 1.57E−03 | −0.303785658 | 0.303785658 | loss | 0.396830715 | 0.093045057 | ESYT3 | 37 | 3 |
| cg24466377 | 9.23E−10 | 3.92E−04 | −0.303770914 | 0.303770914 | loss | 0.554464291 | 0.250693376 | SIX5 | 37 | 19 |
| cg17105139 | 7.13E−10 | 3.03E−04 | −0.303692483 | 0.303692483 | loss | 0.625815136 | 0.322122653 | LBX2; LBX2-AS1 | 37 | 2 |
| cg17842821 | 7.13E−10 | 3.03E−04 | −0.303670203 | 0.303670203 | loss | 0.610297262 | 0.306627059 | | 37 | 5 |
| cg27379787 | 7.13E−10 | 3.03E−04 | −0.3036016 | 0.3036016 | loss | 0.675451347 | 0.371849747 | | 37 | 2 |
| cg00647658 | 7.13E−10 | 3.03E−04 | −0.303567596 | 0.303567596 | loss | 0.398661747 | 0.095094151 | | 37 | 16 |
| cg19966745 | 7.13E−10 | 3.03E−04 | −0.303511977 | 0.303511977 | loss | 0.484257677 | 0.1807457 | | 37 | 10 |
| cg26233253 | 7.13E−10 | 3.03E−04 | −0.303480104 | 0.303480104 | loss | 0.818116809 | 0.514636706 | SLC35E4 | 37 | 22 |
| cg24992471 | 7.13E−10 | 3.03E−04 | −0.303470085 | 0.303470085 | loss | 0.764544526 | 0.461074441 | NGFR | 37 | 17 |
| cg23602058 | 7.77E−10 | 3.30E−04 | −0.303468581 | 0.303468581 | loss | 0.581859392 | 0.278390812 | ITGA3; ITGA3 | 37 | 17 |
| cg06766016 | 7.13E−10 | 3.03E−04 | −0.303458394 | 0.303458394 | loss | 0.9300636 | 0.626605206 | BTBD19 | 37 | 1 |
| cg08012287 | 7.13E−10 | 3.03E−04 | −0.30343638 | 0.30343638 | loss | 0.734588168 | 0.431151788 | ACTN3; ACTN3; ACTN3 | 37 | 11 |
| cg14160968 | 7.13E−10 | 3.03E−04 | −0.30343502 | 0.30343502 | loss | 0.826239549 | 0.522804529 | | 37 | 4 |
| cg00748494 | 1.99E−09 | 8.43E−04 | −0.30341226 | 0.30341226 | loss | 0.423373458 | 0.119961199 | | 37 | 13 |
| cg08537783 | 7.13E−10 | 3.03E−04 | −0.303409255 | 0.303409255 | loss | 0.419881064 | 0.116471809 | CASZ1; CASZ1 | 37 | 1 |
| cg04752871 | 7.13E−10 | 3.03E−04 | −0.303375239 | 0.303375239 | loss | 0.405863575 | 0.102488336 | | 37 | 2 |
| cg01309213 | 7.13E−10 | 3.03E−04 | −0.30337489 | 0.30337489 | loss | 0.777141208 | 0.473766318 | ARHGDIG; PDIA2 | 37 | 16 |
| cg00868766 | 7.13E−10 | 3.03E−04 | −0.303369895 | 0.303369895 | loss | 0.757776619 | 0.454406724 | | 37 | 15 |
| cg21767759 | 8.47E−10 | 3.60E−04 | −0.303366294 | 0.303366294 | loss | 0.573911858 | 0.270545565 | ITGA3; ITGA3 | 37 | 17 |
| cg19414967 | 7.13E−10 | 3.03E−04 | −0.303327497 | 0.303327497 | loss | 0.502131174 | 0.198803676 | | 37 | 8 |
| cg00364956 | 1.30E−09 | 5.52E−04 | −0.303294396 | 0.303294396 | loss | 0.744393002 | 0.441098606 | SLFN13 | 37 | 17 |
| cg17572255 | 1.83E−09 | 7.75E−04 | −0.30327083 | 0.30327083 | loss | 0.614955036 | 0.311684206 | LIMA1; LIMA1 | 37 | 12 |
| cg20048521 | 7.13E−10 | 3.03E−04 | −0.303244199 | 0.303244199 | loss | 0.713466964 | 0.410222765 | KHDC3L | 37 | 6 |
| cg01343041 | 7.13E−10 | 3.03E−04 | −0.303236252 | 0.303236252 | loss | 0.515729817 | 0.212493565 | FAM228A | 37 | 2 |
| cg11460035 | 7.13E−10 | 3.03E−04 | −0.303234513 | 0.303234513 | loss | 0.887052277 | 0.583817765 | LOC643037 | 37 | 11 |
| cg22451657 | 7.13E−10 | 3.03E−04 | −0.303137143 | 0.303137143 | loss | 0.725698813 | 0.422561671 | PLEKHG5; PLEKHG5; PLEKHG5 | 37 | 1 |
| cg25125298 | 7.13E−10 | 3.03E−04 | −0.30309333 | 0.30309333 | loss | 0.392855426 | 0.089762096 | | 37 | 5 |
| cg00463502 | 7.13E−10 | 3.03E−04 | −0.303013112 | 0.303013112 | loss | 0.784116436 | 0.481103324 | WFIKKN2 | 37 | 17 |
| cg22462726 | 4.21E−09 | 1.79E−03 | −0.302961382 | 0.302961382 | loss | 0.906961406 | 0.604000024 | | 37 | 3 |
| cg25926515 | 7.13E−10 | 3.03E−04 | −0.302942132 | 0.302942132 | loss | 0.395735955 | 0.092793822 | TET1 | 37 | 10 |
| cg03127558 | 7.13E−10 | 3.03E−04 | −0.302924419 | 0.302924419 | loss | 0.973194166 | 0.670269747 | MAMSTR | 37 | 19 |
| cg15042332 | 1.43E−08 | 6.06E−03 | −0.302909039 | 0.302909039 | loss | 0.619382109 | 0.316473071 | FABP7 | 37 | 6 |
| cg06710596 | 7.13E−10 | 3.03E−04 | −0.302871706 | 0.302871706 | loss | 0.8238634 | 0.520991694 | GAPDHS | 37 | 19 |
| cg21797718 | 7.13E−10 | 3.03E−04 | −0.302740471 | 0.302740471 | loss | 0.80216933 | 0.499428859 | GRM4; GRM4; GRM4; GRM4; | 37 | 6 |
| | | | | | | | | GRM4; GRM4 | | |
| cg05857999 | 1.81E−08 | 7.69E−03 | −0.302738486 | 0.302738486 | loss | 0.51351434 | 0.210775854 | | 37 | 6 |
| cg03961824 | 7.34E−08 | 3.12E−02 | −0.302731305 | 0.302731305 | loss | 0.773939328 | 0.471208024 | | 37 | 8 |
| cg02213822 | 7.13E−10 | 3.03E−04 | −0.302724544 | 0.302724544 | loss | 0.747585674 | 0.444861129 | | 37 | 9 |
| cg09259081 | 1.68E−09 | 7.12E−04 | −0.302703621 | 0.302703621 | loss | 0.522978985 | 0.220275364 | TLDC1 | 37 | 16 |
| cg11181985 | 7.13E−10 | 3.03E−04 | −0.302640786 | 0.302640786 | loss | 0.828349162 | 0.525708376 | | 37 | 9 |
| cg27491557 | 7.13E−10 | 3.03E−04 | −0.30260064 | 0.30260064 | loss | 0.832465834 | 0.529865194 | | 37 | 6 |
| cg13641043 | 7.13E−10 | 3.03E−04 | −0.302596279 | 0.302596279 | loss | 0.351580889 | 0.048984609 | PLCD3 | 37 | 17 |
| cg03058346 | 1.42E−09 | 6.01E−04 | −0.302572557 | 0.302572557 | loss | 0.732315857 | 0.4297433 | | 37 | 1 |
| cg27483455 | 7.13E−10 | 3.03E−04 | −0.30254432 | 0.30254432 | loss | 0.965246691 | 0.662702371 | ASIC1; ASIC1; ASIC1; ASIC1 | 37 | 12 |
| cg17204557 | 7.13E−10 | 3.03E−04 | −0.302493656 | 0.302493656 | loss | 0.891239309 | 0.588745653 | LINC01101; LINC01101 | 37 | 2 |
| cg14434213 | 7.77E−10 | 3.30E−04 | −0.30248828 | 0.30248828 | loss | 0.686534868 | 0.384046588 | | 37 | 8 |
| cg05376267 | 1.73E−09 | 7.34E−04 | −0.30245351 | 0.30245351 | loss | 0.39493253 | 0.09247902 | LEFTY1 | 37 | 1 |
| cg02625638 | 7.13E−10 | 3.03E−04 | −0.302451469 | 0.302451469 | loss | 0.821437198 | 0.518985729 | FAM83A; FAM83A | 37 | 8 |
| cg16749456 | 8.47E−10 | 3.60E−04 | −0.302351325 | 0.302351325 | loss | 0.733015555 | 0.430664229 | TRPM4; TRPM4 | 37 | 19 |
| cg17768691 | 7.13E−10 | 3.03E−04 | −0.302351165 | 0.302351165 | loss | 0.477123283 | 0.174772118 | PLBD1; PLBD1 | 37 | 12 |
| cg15667844 | 7.13E−10 | 3.03E−04 | −0.302286617 | 0.302286617 | loss | 0.50077787 | 0.198491253 | DUSP5 | 37 | 10 |
| cg22522920 | 7.13E−10 | 3.03E−04 | −0.302174198 | 0.302174198 | loss | 0.604279857 | 0.302105659 | FAIM2 | 37 | 12 |
| cg01001533 | 7.13E−10 | 3.03E−04 | −0.302149306 | 0.302149306 | loss | 0.502173936 | 0.200024629 | | 37 | 10 |
| cg04686778 | 7.13E−10 | 3.03E−04 | −0.302141226 | 0.302141226 | loss | 0.985175796 | 0.683034571 | NRXN2; NRXN2; NRXN2 | 37 | 11 |
| cg25541708 | 7.13E−10 | 3.03E−04 | −0.302035046 | 0.302035046 | loss | 0.937650587 | 0.635615541 | | 37 | 5 |
| cg00034101 | 9.23E−10 | 3.92E−04 | −0.302029877 | 0.302029877 | loss | 0.796383077 | 0.4943532 | HEYL | 37 | 1 |
| cg10838157 | 7.77E−10 | 3.30E−04 | −0.302024793 | 0.302024793 | loss | 0.680567887 | 0.378543094 | | 37 | 15 |
| cg16971128 | 7.13E−10 | 3.03E−04 | −0.302010892 | 0.302010892 | loss | 0.882536092 | 0.5805252 | TMPRSS6; TMPRSS6 | 37 | 22 |
| cg22573675 | 7.13E−10 | 3.03E−04 | −0.301999376 | 0.301999376 | loss | 0.913898223 | 0.611898847 | | 37 | 2 |
| cg11386011 | 7.13E−10 | 3.03E−04 | −0.301996439 | 0.301996439 | loss | 0.809303915 | 0.507307476 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg26500033 | 8.80E−09 | 3.74E−03 | −0.301949565 | 0.301949565 | loss | 0.8141436 | 0.512194035 | | 37 | 6 |
| cg25308803 | 3.88E−09 | 1.65E−03 | −0.301890312 | 0.301890312 | loss | 0.647561683 | 0.345671371 | SH3RF3; SH3RF3- | 37 | 2 |
| | | | | | | | | AS1; SH3RF3-AS1 | | |
| cg15891218 | 1.30E−09 | 5.52E−04 | −0.301865686 | 0.301865686 | loss | 0.777800057 | 0.475934371 | TMEM308; TMEM308 | 37 | 14 |
| cg11362935 | 7.77E−10 | 3.30E−04 | −0.301758503 | 0.301758503 | loss | 0.446822028 | 0.145063525 | POU2AF1; POU2AF1 | 37 | 11 |
| cg23009355 | 7.13E−10 | 3.03E−04 | −0.301662596 | 0.301662596 | loss | 0.967077802 | 0.665415206 | GAL | 37 | 11 |
| cg14099457 | 7.77E−10 | 3.30E−04 | −0.301589434 | 0.301589434 | loss | 0.53771584 | 0.236126406 | LAMB2; LAMB2 | 37 | 3 |
| cg04585822 | 8.47E−10 | 3.60E−04 | −0.301588243 | 0.301588243 | loss | 0.763370066 | 0.461781824 | USH1G; USH1G | 37 | 17 |
| cg03014829 | 7.13E−10 | 3.03E−04 | −0.301554748 | 0.301554748 | loss | 0.69198813 | 0.390433382 | | 37 | 7 |
| cg13707623 | 7.13E−10 | 3.03E−04 | −0.301550977 | 0.301550977 | loss | 0.950002525 | 0.648451547 | ZNF831 | 37 | 20 |
| cg00984540 | 1.83E−09 | 7.75E−04 | −0.301514152 | 0.301514152 | loss | 0.91544837 | 0.613934218 | | 37 | 7 |
| cg13214295 | 7.13E−10 | 3.03E−04 | −0.301480874 | 0.301480874 | loss | 0.582890757 | 0.281409882 | | 37 | 17 |
| cg17324149 | 7.13E−10 | 3.03E−04 | −0.301384421 | 0.301384421 | loss | 0.555203809 | 0.253819388 | | 37 | 3 |
| cg19833103 | 7.11E−10 | 3.03E−04 | −0.301373283 | 0.301373283 | loss | 0.44962123 | 0.148247947 | LRCH1; LRCH1; LRCH1 | 37 | 13 |
| cg26672426 | 7.13E−10 | 3.03E−04 | −0.301366266 | 0.301366266 | loss | 0.702441225 | 0.401074959 | PTGES | 37 | 9 |
| cg11166453 | 1.10E−09 | 4.65E−04 | −0.301346531 | 0.301346531 | loss | 0.664128472 | 0.362781941 | GCSAML; GCSAML; GCSAML; | 37 | 1 |
| | | | | | | | | GCSAML; GCSAML; GCSAML | | |
| cg07951355 | 7.13E−10 | 3.03E−04 | −0.30132906 | 0.30132906 | loss | 0.498254019 | 0.196924959 | | 37 | 1 |
| cg01382860 | 7.13E−10 | 3.03E−04 | −0.30130727 | 0.30130727 | loss | 0.899197623 | 0.597890353 | TRPM4; TRPM4 | 37 | 19 |
| cg04327247 | 7.13E−10 | 3.03E−04 | −0.301206563 | 0.301206563 | loss | 0.858338551 | 0.557131988 | | 37 | 9 |
| cg25478132 | 7.13E−10 | 3.03E−04 | −0.301147568 | 0.301147568 | loss | 0.894313468 | 0.5931659 | TLX1NB | 37 | 10 |
| cg01440864 | 7.13E−10 | 3.03E−04 | −0.301142278 | 0.301142278 | loss | 0.71928936 | 0.418147082 | WNT3 | 37 | 17 |
| cg04986934 | 7.13E−10 | 3.03E−04 | −0.301108743 | 0.301108743 | loss | 0.86008766 | 0.558978918 | KRT12 | 37 | 17 |
| cg13067553 | 7.13E−10 | 3.03E−04 | −0.301093352 | 0.301093352 | loss | 0.439625092 | 0.13853174 | NBPF8; NBPF8; NBPF8 | 37 | 1 |
| cg19552640 | 2.35E−09 | 9.98E−04 | −0.301086551 | 0.301086551 | loss | 0.614538751 | 0.3134522 | MEGF10; MEGF10 | 37 | 5 |
| cg05308656 | 7.13E−10 | 3.03E−04 | −0.301019327 | 0.301019327 | loss | 0.436557694 | 0.135538368 | ARL4C; ARL4C | 37 | 2 |
| cg26772540 | 8.47E−10 | 3.60E−04 | −0.300971687 | 0.300971687 | gain | 0.241402208 | 0.542373894 | ZNF833P | 37 | 19 |
| cg18770763 | 7.13E−10 | 3.03E−04 | −0.300951045 | 0.300951045 | loss | 0.706449951 | 0.405498906 | EPS8L2 | 37 | 11 |
| cg09119043 | 1.01E−09 | 4.27E−04 | −0.30093308 | 0.30093308 | loss | 0.571926509 | 0.270993429 | SLC35F3 | 37 | 1 |
| cg10257870 | 7.77E−10 | 3.30E−04 | −0.300917374 | 0.300917374 | loss | 0.692200815 | 0.391283441 | CMYA5 | 37 | 5 |
| cg23882131 | 7.13E−10 | 3.03E−04 | −0.300842395 | 0.300842395 | loss | 0.74249943 | 0.441657035 | MRGPRF; MRGPRF | 37 | 11 |
| cg15587771 | 1.99E−09 | 8.43E−04 | −0.300794471 | 0.300794471 | loss | 0.736634589 | 0.435840118 | SDK1 | 37 | 7 |
| cg24210717 | 7.13E−10 | 3.03E−04 | −0.300785027 | 0.300785027 | loss | 0.684959304 | 0.384174276 | GPD1; GPD1 | 37 | 12 |
| cg13948430 | 7.13E−10 | 3.03E−04 | −0.30077466 | 0.30077466 | loss | 0.840957619 | 0.540182959 | | 37 | 2 |
| cg02150077 | 1.42E−09 | 6.01E−04 | −0.300749108 | 0.300749106 | loss | 0.626169723 | 0.325420616 | | 37 | 16 |
| cg06679334 | 7.13E−10 | 3.03E−04 | −0.300712507 | 0.300712507 | loss | 0.449281142 | 0.148568634 | | 37 | 17 |
| cg00697301 | 1.89E−09 | 8.02E−04 | −0.300710036 | 0.300710036 | loss | 0.875718998 | 0.575008963 | | 37 | 12 |
| cg24258347 | 1.19E−09 | 5.07E−04 | −0.300685804 | 0.300685804 | loss | 0.816674487 | 0.515988682 | SPON1 | 37 | 11 |
| cg16768191 | 7.77E−10 | 3.30E−04 | −0.300621446 | 0.300621446 | loss | 0.79456594 | 0.493944494 | | 37 | 7 |
| cg21714557 | 7.13E−10 | 3.03E−04 | −0.300611833 | 0.300611833 | loss | 0.865727668 | 0.565115835 | F10 | 37 | 13 |
| cg14132536 | 7.13E−10 | 3.03E−04 | −0.300610682 | 0.300610682 | loss | 0.501199 | 0.200588318 | DPM2 | 37 | 9 |
| cg20339859 | 7.77E−10 | 3.30E−04 | −0.300594675 | 0.300594675 | loss | 0.771911698 | 0.471317024 | | 37 | 10 |
| cg06012872 | 7.13E−10 | 3.03E−04 | −0.300555263 | 0.300555263 | loss | 0.446083292 | 0.145528029 | TMEM72-AS1 | 37 | 10 |
| cg24517467 | 1.42E−09 | 6.01E−04 | −0.300540437 | 0.300540437 | loss | 0.476160015 | 0.175619578 | | 37 | 7 |
| cg17401780 | 7.13E−10 | 3.03E−04 | −0.300528324 | 0.300528324 | loss | 0.837540783 | 0.537012459 | | 37 | 10 |
| cg16390049 | 7.84E−10 | 3.33E−04 | −0.300484998 | 0.300484998 | loss | 0.881432181 | 0.580947182 | NOTCH4 | 37 | 6 |
| cg19497444 | 1.68E−09 | 7.12E−04 | −0.300469473 | 0.300469473 | loss | 0.699317696 | 0.398848224 | SLC22A18; SLC22A18 | 37 | 11 |
| cg13695585 | 7.13E−10 | 3.03E−04 | −0.300363585 | 0.300363585 | loss | 0.736149874 | 0.435786288 | DDR1; DDR1; DDR1 | 37 | 6 |
| cg15682101 | 7.13E−10 | 3.03E−04 | −0.300261907 | 0.300261907 | loss | 0.613984425 | 0.313722518 | | 37 | 6 |
| cg14896134 | 7.13E−10 | 3.03E−04 | −0.300205578 | 0.300205578 | loss | 0.472272789 | 0.172067211 | | 37 | 10 |
| cg13666323 | 3.28E−09 | 1.39E−03 | −0.300184632 | 0.300184632 | loss | 0.536153936 | 0.235969304 | | 37 | X |
| cg21644489 | 7.13E−10 | 3.03E−04 | −0.300184417 | 0.300184417 | loss | 0.818662323 | 0.518477906 | | 37 | 15 |
| cg02760962 | 4.64E−08 | 1.97E−02 | −0.300179972 | 0.300179972 | loss | 0.736755819 | 0.436575847 | TRIM44 | 37 | 11 |
| cg07356483 | 1.99E−09 | 8.43E−04 | −0.300158545 | 0.300158545 | loss | 0.718617662 | 0.418459118 | CRTAC1; CRTAC1 | 37 | 10 |
| cg12288076 | 7.13E−10 | 3.03E−04 | −0.300148377 | 0.300148377 | loss | 0.587979236 | 0.287830859 | OCSTAMP | 37 | 20 |
| cg25606780 | 7.13E−10 | 3.03E−04 | −0.30011983 | 0.30011983 | loss | 0.951157283 | 0.651037453 | | 37 | 3 |
| cg02206373 | 1.19E−09 | 5.07E−04 | −0.300095908 | 0.300095908 | loss | 0.862087308 | 0.5619914 | STEAP2; STEAP2; STEAP2; STEAP2; | 37 | 7 |
| | | | | | | | | STEAP2; STEAP2 | | |
| cg11434671 | 7.13E−10 | 3.03E−04 | −0.300091613 | 0.300091613 | loss | 0.580022177 | 0.279930565 | CYGB; PRCD | 37 | 17 |
| cg09068286 | 7.13E−10 | 3.03E−04 | −0.300016317 | 0.300016317 | loss | 0.575604264 | 0.275587947 | GFPT2 | 37 | 5 |
| cg16290689 | 7.13E−10 | 3.03E−04 | −0.300002651 | 0.300002651 | loss | 0.582751187 | 0.282748535 | TEX26; TEX26-AS1; TEX26- | 37 | 13 |
| | | | | | | | | AS1; TEX26-AS1; TEX26- | | |
| | | | | | | | | AS1 | | |
| cg03441844 | 7.13E−10 | 3.03E−04 | −0.299914998 | 0.299914998 | loss | 0.436974774 | 0.137059776 | | 37 | 1 |
| cg21225804 | 7.13E−10 | 3.03E−04 | −0.299903498 | 0.299903498 | loss | 0.51138434 | 0.211480841 | NCS1 | 37 | 9 |
| cg11822175 | 7.13E−10 | 3.03E−04 | −0.299899539 | 0.299899539 | loss | 0.891839704 | 0.591940165 | | 37 | 10 |
| cg15203566 | 1.10E−09 | 4.65E−04 | −0.299881847 | 0.299881847 | loss | 0.889356111 | 0.589474265 | | 37 | 5 |
| cg08272268 | 7.13E−10 | 3.03E−04 | −0.299782464 | 0.299782464 | loss | 0.659622464 | 0.35984 | ZNF281; ZNF281; ZNF281 | 37 | 1 |
| cg17726692 | 3.67E−08 | 1.56E−02 | −0.299693566 | 0.299693566 | loss | 0.817074166 | 0.5173806 | LINC00908 | 37 | 18 |
| cg15043318 | 7.13E−10 | 3.03E−04 | −0.299677133 | 0.299677133 | loss | 0.902996968 | 0.603319835 | | 37 | 5 |
| cg19143959 | 7.13E−10 | 3.03E−04 | −0.299639031 | 0.299639031 | loss | 0.916291849 | 0.616652818 | | 37 | 15 |
| cg24341311 | 8.47E−10 | 3.60E−04 | −0.299605965 | 0.299605965 | loss | 0.849795536 | 0.550189571 | C3orf56 | 37 | 3 |
| cg27331665 | 7.13E−10 | 3.03E−04 | −0.299596534 | 0.299596534 | loss | 0.489150394 | 0.18955386 | | 37 | 11 |
| cg07330481 | 7.13E−10 | 3.03E−04 | −0.299596266 | 0.299596266 | loss | 0.629087008 | 0.329490741 | ARL5C; ARL5C | 37 | 17 |
| cg24694702 | 7.13E−10 | 3.03E−04 | −0.299592182 | 0.299592182 | loss | 0.746227106 | 0.446634924 | HSP88 | 37 | 12 |
| cg02365397 | 7.13E−10 | 3.03E−04 | −0.299570831 | 0.299570831 | loss | 0.78639976 | 0.486828929 | ANKRD63 | 37 | 15 |
| cg08035479 | 2.16E−09 | 9.17E−04 | −0.299539222 | 0.299539222 | loss | 0.843246592 | 0.543707371 | LOC286177 | 37 | 8 |
| cg01168289 | 9.34E−10 | 3.97E−04 | −0.299478046 | 0.299478046 | loss | 0.895027429 | 0.595549382 | WT1; WT1; WT1; WT1; WT1 | 37 | 11 |
| cg12451836 | 7.13E−10 | 3.03E−04 | −0.299455465 | 0.299455465 | loss | 0.456285694 | 0.156830229 | | 37 | 7 |
| cg03832522 | 7.13E−10 | 3.03E−04 | −0.299395609 | 0.299395609 | loss | 0.359803921 | 0.060408312 | RUFY4 | 37 | 2 |
| cg12199221 | 2.48E−08 | 1.05E−02 | −0.299376689 | 0.299376689 | loss | 0.435196243 | 0.135819554 | HAPLN1; HAPLN1 | 37 | 5 |
| cg13445177 | 7.13E−10 | 3.03E−04 | −0.299365918 | 0.299365918 | loss | 0.772163942 | 0.472798024 | S100A10 | 37 | 1 |
| cg11403027 | 7.13E−10 | 3.03E−04 | −0.299245649 | 0.299245649 | loss | 0.883639049 | 0.5843934 | | 37 | 5 |
| cg03340760 | 7.77E−10 | 3.30E−04 | −0.299200057 | 0.299200057 | loss | 0.752164287 | 0.452964229 | | 37 | 2 |
| cg09481121 | 8.47E−10 | 3.60E−04 | −0.299179395 | 0.299179395 | loss | 0.779919525 | 0.480740129 | CMYA5 | 37 | 5 |
| cg14847243 | 7.13E−10 | 3.03E−04 | −0.299167714 | 0.299167714 | loss | 0.670839102 | 0.371671388 | CHRD | 37 | 3 |
| cg08481464 | 7.13E−10 | 3.03E−04 | −0.299142514 | 0.299142514 | loss | 0.749466085 | 0.450323571 | C10orf11 | 37 | 10 |
| cg14897238 | 8.47E−10 | 3.60E−04 | −0.299121068 | 0.299121068 | loss | 0.469877662 | 0.170756594 | | 37 | 21 |
| cg14972155 | 2.91E−08 | 1.23E−02 | −0.299113233 | 0.299113233 | loss | 0.704495457 | 0.405382224 | ESM1; ESM1 | 37 | 5 |
| cg22624278 | 7.13E−10 | 3.03E−04 | −0.299087434 | 0.299087434 | loss | 0.877481575 | 0.578394141 | | 37 | 12 |
| cg07164191 | 7.13E−10 | 3.03E−04 | −0.298994484 | 0.298994484 | loss | 0.834586408 | 0.535591924 | | 37 | 5 |
| cg18556455 | 7.13E−10 | 3.03E−04 | −0.298941726 | 0.298941726 | loss | 0.768234196 | 0.469292471 | | 37 | 2 |
| cg03969301 | 7.77E−10 | 3.30E−04 | −0.298934318 | 0.298934318 | loss | 0.861393736 | 0.562459418 | | 37 | 2 |
| cg24067118 | 7.13E−10 | 3.03E−04 | −0.298910858 | 0.298910858 | loss | 0.721443464 | 0.422532606 | | 37 | 10 |
| cg23647410 | 7.13E−10 | 3.03E−04 | −0.298880098 | 0.298880098 | loss | 0.817137345 | 0.518257247 | | 37 | 1 |
| cg03484180 | 7.13E−10 | 3.03E−04 | −0.298876823 | 0.298876823 | loss | 0.46893076 | 0.170053937 | MON1A; MON1A; MON1A; | 37 | 3 |
| | | | | | | | | MON1A | | |
| cg25966908 | 7.77E−10 | 3.30E−04 | −0.298863672 | 0.298863672 | loss | 0.521266872 | 0.2224032 | | 37 | 7 |
| cg17469265 | 1.10E−09 | 4.65E−04 | −0.298834491 | 0.298834491 | loss | 0.629003591 | 0.3301691 | WDR19 | 37 | 4 |
| cg06468108 | 8.47E−10 | 3.60E−04 | −0.298808149 | 0.298808149 | loss | 0.577906743 | 0.279098594 | | 37 | 2 |
| cg01971227 | 7.13E−10 | 3.03E−04 | −0.298705701 | 0.298705701 | loss | 0.414194855 | 0.115489154 | | 37 | 16 |
| cg10487067 | 7.13E−10 | 3.03E−04 | −0.298590731 | 0.298590731 | loss | 0.614095649 | 0.315504918 | | 37 | 1 |
| cg17745234 | 7.13E−10 | 3.03E−04 | −0.298543685 | 0.298543685 | loss | 0.701922379 | 0.403378694 | TMPRSS6; TMPRSS6 | 37 | 22 |
| cg18460422 | 7.13E−10 | 3.03E−04 | −0.29853524 | 0.29853524 | loss | 0.774542887 | 0.476007647 | TNXB | 37 | 6 |
| cg02024846 | 7.13E−10 | 3.03E−04 | −0.29851901 | 0.29851901 | loss | 0.569872734 | 0.271353724 | CDC14B; CDC14B | 37 | 9 |
| cg25394203 | 7.13E−10 | 3.03E−04 | −0.298510645 | 0.298510645 | gain | 0.197819555 | 0.4963302 | ZNF833P | 37 | 19 |
| cg19149031 | 7.13E−10 | 3.03E−04 | −0.29846202 | 0.29846202 | loss | 0.879230949 | 0.580768929 | SLC22A3 | 37 | 6 |
| cg22471112 | 1.01E−09 | 4.27E−04 | −0.298437255 | 0.298437255 | loss | 0.677546655 | 0.3791094 | TRIM2; TRIM2 | 37 | 4 |
| cg23626733 | 7.13E−10 | 3.03E−04 | −0.298424027 | 0.298424027 | loss | 0.751890015 | 0.453465988 | PEX10; PEX10 | 37 | 1 |
| cg13337731 | 7.13E−10 | 3.03E−04 | −0.298394669 | 0.298394669 | loss | 0.449841534 | 0.151446865 | MLXIPL; MLXIPL; MIXIPL; | 37 | 7 |
| cg22445712 | 7.13E−10 | 3.03E−04 | −0.298369284 | 0.298369284 | loss | 0.481113513 | 0.182744229 | PLXNB2 | 37 | 22 |
| cg27385590 | 1.10E−09 | 4.65E−04 | −0.298309538 | 0.298309538 | loss | 0.527370774 | 0.229061235 | | 37 | 7 |
| cg05499178 | 7.13E−10 | 3.03E−04 | −0.298305604 | 0.298305604 | loss | 0.682912598 | 0.384606994 | | 37 | 11 |
| cg03697173 | 7.13E−10 | 3.03E−04 | −0.298282667 | 0.298282667 | loss | 0.833170391 | 0.534887724 | RADIL | 37 | 7 |
| cg11676353 | 7.13E−10 | 3.03E−04 | −0.298073451 | 0.298073451 | loss | 0.85239454 | 0.554321088 | | 37 | 1 |
| cg01811796 | 7.13E−10 | 3.03E−04 | −0.298053747 | 0.298053747 | loss | 0.426842779 | 0.128789032 | STMN1; STMN1; STMN1; STMN1; | 37 | 1 |
| | | | | | | | | STMN1; MIR3917 | | |
| cg10217202 | 7.13E−10 | 3.03E−04 | −0.298031783 | 0.298031783 | loss | 0.854814377 | 0.556782594 | | 37 | 14 |
| cg15302379 | 8.47E−10 | 3.60E−04 | −0.298025073 | 0.298025073 | loss | 0.730877132 | 0.432852059 | KAZALD1 | 37 | 10 |
| cg01499518 | 1.10E−09 | 4.65E−04 | −0.298005033 | 0.298005033 | loss | 0.599594398 | 0.301589365 | SRRM3 | 37 | 7 |
| cg12079699 | 7.13E−10 | 3.03E−04 | −0.297875718 | 0.297875718 | loss | 0.91755823 | 0.619682512 | NTM | 37 | 11 |
| cg24414344 | 7.77E−10 | 3.30E−04 | −0.297858352 | 0.297858352 | loss | 0.617185981 | 0.319327629 | KRT12 | 37 | 17 |
| cg00463859 | 7.13E−10 | 3.03E−04 | −0.297735651 | 0.297735651 | loss | 0.755668451 | 0.4579328 | TCEA3 | 37 | 1 |
| cg17213154 | 1.68E−09 | 7.12E−04 | −0.297705415 | 0.297705415 | loss | 0.758598226 | 0.460892812 | ST5; ST5; ST5; ST5 | 37 | 11 |
| cg22523072 | 9.23E−10 | 3.92E−04 | −0.297630273 | 0.297630273 | loss | 0.698084732 | 0.400454459 | | 37 | 1 |
| cg08231697 | 7.13E−10 | 3.03E−04 | −0.297571924 | 0.297571924 | loss | 0.838022689 | 0.540450765 | | 37 | 1 |
| cg02431260 | 7.13E−10 | 3.03E−04 | −0.29754729 | 0.29754729 | loss | 0.471910987 | 0.174363696 | ESAM | 37 | 11 |
| cg03939719 | 7.13E−10 | 3.03E−04 | −0.297460429 | 0.297460429 | loss | 0.7119571 | 0.414496671 | | 37 | 12 |
| cg24599107 | 7.77E−10 | 3.30E−04 | −0.297439548 | 0.297439548 | loss | 0.91386543 | 0.616425882 | PVRL4 | 37 | 1 |
| cg13220917 | 7.13E−10 | 3.03E−04 | −0.297383507 | 0.297383507 | loss | 0.633584625 | 0.336201118 | | 37 | 2 |
| cg07935320 | 7.13E−10 | 3.03E−04 | −0.297383191 | 0.297383191 | loss | 0.970610238 | 0.673227047 | FAM83A; FAM83A | 37 | 8 |
| cg24279744 | 7.13E−10 | 3.03E−04 | −0.297377043 | 0.297377043 | loss | 0.606053949 | 0.308676906 | ATP2B2; ATP2B2 | 37 | 3 |
| cg12271415 | 7.13E−10 | 3.03E−04 | −0.297369674 | 0.297369674 | loss | 0.696803568 | 0.399433894 | SSC5D; SSC5D | 37 | 19 |
| cg03517083 | 1.83E−09 | 7.75E−04 | −0.29733292 | 0.29733292 | loss | 0.487049117 | 0.189716197 | CHST5 | 37 | 16 |
| cg12793610 | 9.23E−10 | 3.92E−04 | −0.297309432 | 0.297309432 | loss | 0.757423985 | 0.460114553 | TRIM2; TRIM2 | 37 | 4 |
| cg22550309 | 1.68E−09 | 7.12E−04 | −0.297248486 | 0.297248486 | loss | 0.949859004 | 0.652610518 | PRSS42 | 37 | 3 |
| cg19773546 | 7.13E−10 | 3.03E−04 | −0.297165753 | 0.297165753 | loss | 0.976683994 | 0.679518241 | | 37 | 17 |
| cg18045195 | 7.13E−10 | 3.03E−04 | −0.297153292 | 0.297153292 | loss | 0.583607309 | 0.286454018 | | 37 | 2 |
| cg20800117 | 4.64E−08 | 1.97E−02 | −0.297151207 | 0.297151207 | loss | 0.677472783 | 0.380321576 | CAMTA1 | 37 | 1 |
| cg26931208 | 7.13E−10 | 3.03E−04 | −0.297143517 | 0.297143517 | loss | 0.661279911 | 0.364136394 | RASIP1; IZUMO1 | 37 | 19 |
| cg09411356 | 7.13E−10 | 3.03E−04 | −0.297095685 | 0.297095685 | loss | 0.800235543 | 0.503139859 | OBSCN; OBSCN; OBSCN; C1orf145; | 37 | 1 |
| | | | | | | | | C1orf145 | | |
| cg06351981 | 7.13E−10 | 3.03E−04 | −0.297064164 | 0.297064164 | loss | 0.722582223 | 0.425518059 | SPEG | 37 | 2 |
| cg16686158 | 7.84E−10 | 3.33E−04 | −0.297064056 | 0.297064056 | loss | 0.458011687 | 0.160947631 | HORMAD2 | 37 | 22 |
| cg10800953 | 1.73E−09 | 7.34E−04 | −0.297060432 | 0.297060432 | loss | 0.723640989 | 0.426580556 | CHRD | 37 | 3 |
| cg13477780 | 7.77E−10 | 3.30E−04 | −0.297026989 | 0.297026989 | loss | 0.50183753 | 0.204810541 | SRRM3 | 37 | 7 |
| cg00701706 | 7.13E−10 | 3.03E−04 | −0.296987674 | 0.296987674 | loss | 0.589133157 | 0.292145482 | OTOP3; OTOP3 | 37 | 17 |
| cg01759114 | 2.12E−08 | 9.01E−03 | −0.296973589 | 0.296973589 | loss | 0.601213842 | 0.304240253 | | 37 | 8 |
| cg11688949 | 2.16E−09 | 9.17E−04 | −0.296954712 | 0.296954712 | loss | 0.513491489 | 0.216536776 | STMN4; STMN4; STMN4; STMN4; | 37 | 8 |
| | | | | | | | | STMN4; STMN4; STMN4; | | |
| | | | | | | | | STMN4 | | |
| cg10365312 | 7.13E−10 | 3.03E−04 | −0.29695275 | 0.29695275 | loss | 0.839998785 | 0.543046035 | WFDC5 | 37 | 20 |
| cg26019584 | 7.13E−10 | 3.03E−04 | −0.296925656 | 0.296925656 | loss | 0.857556721 | 0.560631065 | | 37 | 19 |
| cg00537837 | 7.13E−10 | 3.03E−04 | −0.296874045 | 0.296874045 | loss | 0.639389845 | 0.3425158 | | 37 | 16 |
| cg01283332 | 1.12E−08 | 4.76E−03 | −0.296871864 | 0.296871864 | loss | 0.630057228 | 0.333185365 | | 37 | 5 |
| cg18798774 | 8.47E−10 | 3.60E−04 | −0.296867225 | 0.296867225 | loss | 0.887345266 | 0.590478041 | HSPA12A | 37 | 10 |
| cg04631458 | 7.13E−10 | 3.03E−04 | −0.296864128 | 0.296864128 | loss | 0.451404581 | 0.154540453 | | 37 | 7 |
| cg05888252 | 7.13E−10 | 3.03E−04 | −0.296851864 | 0.296851864 | loss | 0.827010817 | 0.530158953 | | 37 | 1 |
| cg16894439 | 9.23E−10 | 3.92E−04 | −0.296846694 | 0.296846694 | loss | 0.699032858 | 0.402186165 | | 37 | 1 |
| cg22172057 | 1.83E−09 | 7.75E−04 | −0.296774823 | 0.296774823 | loss | 0.448625011 | 0.151850188 | ADAR82 | 37 | 10 |
| cg00976097 | 1.10E−09 | 4.65E−04 | −0.296772819 | 0.296772819 | loss | 0.914505672 | 0.617732853 | AHRR; AHRR | 37 | 5 |
| cg07586026 | 7.13E−10 | 3.03E−04 | −0.296758566 | 0.296758566 | loss | 0.490033625 | 0.193275059 | | 37 | 1 |
| cg05714552 | 7.13E−10 | 3.03E−04 | −0.296743869 | 0.296743869 | loss | 0.400566162 | 0.103822293 | PRRT1 | 37 | 6 |
| cg26174326 | 7.13E−10 | 3.03E−04 | −0.29671486 | 0.29671486 | loss | 0.95239763 | 0.655682771 | | 37 | 6 |
| cg10503232 | 7.13E−10 | 3.03E−04 | −0.296706425 | 0.296706425 | loss | 0.775619508 | 0.478913082 | KCNQ1DN | 37 | 11 |
| cg27244482 | 8.47E−10 | 3.60E−04 | −0.296702665 | 0.296702665 | loss | 0.759843347 | 0.463140682 | CAMK2A; CAMK2A; CAMK2A; | 37 | 5 |
| | | | | | | | | CAMK2A | | |
| cg00603340 | 7.13E−10 | 3.03E−04 | −0.296695495 | 0.296695495 | loss | 0.619926677 | 0.323231182 | TRIM7,TRIM7; TRIM7; TRIM7; | 37 | 5 |
| | | | | | | | | TRIM7 | | |
| cg20958628 | 7.13E−10 | 3.03E−04 | −0.296563446 | 0.296563446 | loss | 0.868096087 | 0.571532641 | KAZN; KAZN; KAZN | 37 | 1 |
| cg19098932 | 3.18E−09 | 1.35E−03 | −0.296558836 | 0.296558836 | loss | 0.499271396 | 0.20271256 | PEX10; PEX10 | 37 | 1 |
| cg19590598 | 7.13E−10 | 3.03E−04 | −0.296539426 | 0.296539426 | loss | 0.516740796 | 0.220201371 | | 37 | 2 |
| cg24024528 | 7.92E−08 | 3.36E−02 | −0.296523167 | 0.296523167 | loss | 0.788607779 | 0.492084612 | | 37 | 12 |
| cg02400092 | 7.84E−10 | 3.33E−04 | −0.296485761 | 0.296485761 | loss | 0.327376919 | 0.030891158 | ZBTB44 | 37 | 11 |
| cg10866062 | 7.13E−10 | 3.03E−04 | −0.296475992 | 0.296475992 | loss | 0.503349751 | 0.206873759 | BTBD17 | 37 | 17 |
| cg23851167 | 7.13E−10 | 3.03E−04 | −0.296471024 | 0.296471024 | loss | 0.700425194 | 0.403954171 | EEF1A2 | 37 | 20 |
| cg19146112 | 7.13E−10 | 3.03E−04 | −0.296442413 | 0.296442413 | loss | 0.874646925 | 0.578204512 | KHDC3L | 37 | 6 |
| cg18224262 | 7.13E−10 | 3.03E−04 | −0.296359244 | 0.296359244 | loss | 0.726812268 | 0.430453024 | | 37 | 18 |
| cg17434154 | 7.13E−10 | 3.03E−04 | −0.296266379 | 0.296266379 | loss | 0.581542826 | 0.285276447 | ESRRG | 37 | 1 |
| cg20091689 | 7.13E−10 | 3.03E−04 | −0.296264717 | 0.296264717 | loss | 0.789788711 | 0.493523994 | ANKRD39 | 37 | 2 |
| cg03521737 | 7.13E−10 | 3.03E−04 | −0.296261616 | 0.296261616 | loss | 0.560851698 | 0.264590082 | | 37 | 1 |
| cg27302994 | 1.01E−09 | 4.27E−04 | −0.296210166 | 0.296210166 | loss | 0.677357166 | 0.381147 | | 37 | 1 |
| cg05390307 | 7.13E−10 | 3.03E−04 | −0.296203612 | 0.296203612 | loss | 0.831195353 | 0.534991741 | FEZ1; FEZ1 | 37 | 11 |
| cg24360241 | 7.13E−10 | 3.03E−04 | −0.29618292 | 0.29618292 | loss | 0.520063349 | 0.223880429 | | 37 | 2 |
| cg08572336 | 7.13E−10 | 3.03E−04 | −0.296179697 | 0.296179697 | loss | 0.864594221 | 0.568414524 | SHANK1 | 37 | 19 |
| cg06419659 | 1.83E−09 | 7.75E−04 | −0.296057153 | 0.296057153 | loss | 0.394172217 | 0.098115064 | S100A13; S100A13; S100A13; | 37 | 1 |
| | | | | | | | | S100A13; S100A13; S100A13; | | |
| | | | | | | | | S100A1 | | |
| cg14263391 | 7.13E−10 | 3.03E−04 | −0.296039523 | 0.296039523 | loss | 0.510874158 | 0.214834635 | GRB7; GRB7; GRB7 | 37 | 17 |
| cg25763520 | 7.13E−10 | 3.03E−04 | −0.295939608 | 0.295939608 | loss | 0.923302296 | 0.627362688 | PRPH | 37 | 12 |
| cg14508777 | 7.13E−10 | 3.03E−04 | −0.295909826 | 0.295909826 | loss | 0.678118685 | 0.382208859 | HIGD1A; HIGD1A; HIGD1A | 37 | 3 |
| cg04198698 | 7.13E−10 | 3.03E−04 | −0.295878363 | 0.295878363 | loss | 0.865656498 | 0.569778135 | | 37 | 15 |
| cg24152179 | 7.13E−10 | 3.03E−04 | −0.295846439 | 0.295846439 | loss | 0.848329745 | 0.552483306 | TBX1; TBX1; TBX1 | 37 | 22 |
| cg22986999 | 7.13E−10 | 3.03E−04 | −0.29583291 | 0.29583291 | loss | 0.437052604 | 0.141219694 | MRGPRF; MRGPRF | 37 | 11 |
| cg08421934 | 8.80E−09 | 3.74E−03 | −0.295724289 | 0.295724289 | loss | 0.62669783 | 0.330973541 | | 37 | 6 |
| cg10202113 | 3.88E−09 | 1.65E−03 | −0.295677492 | 0.295677492 | loss | 0.697838909 | 0.402161418 | | 37 | 1 |
| cg09636313 | 7.13E−10 | 3.03E−04 | −0.295629187 | 0.295629187 | loss | 0.691662134 | 0.396032947 | | 37 | 16 |
| cg17382841 | 8.56E−10 | 3.63E−04 | −0.295602098 | 0.295602098 | loss | 0.78755351 | 0.491951412 | EGFLAM; EGFLAM; EGFLAM; | 37 | 5 |
| | | | | | | | | EGFLAM | | |
| cg02413092 | 7.13E−10 | 3.03E−04 | −0.295477807 | 0.295477807 | loss | 0.60608273 | 0.310604924 | NOM1 | 37 | 7 |
| cg05586134 | 7.13E−10 | 3.03E−04 | −0.295462194 | 0.295462194 | loss | 0.978096258 | 0.682634065 | PTCRA; PTCRA; PTCRA; PTCRA | 37 | 6 |
| cg10270430 | 7.13E−10 | 3.03E−04 | −0.295398701 | 0.295398701 | loss | 0.747827319 | 0.452428618 | GRM4; GRM4; GRM4; GRM4; | 37 | 6 |
| | | | | | | | | GRM4; GRM4 | | |
| cg02989255 | 7.13E−10 | 3.03E−04 | −0.295397867 | 0.295397867 | loss | 0.660250196 | 0.364852329 | TNXB | 37 | 6 |
| cg00337187 | 7.13E−10 | 3.03E−04 | −0.295332625 | 0.295332625 | loss | 0.575945372 | 0.280612747 | | 37 | 6 |
| cg07296387 | 8.11E−09 | 3.44E−03 | −0.295329812 | 0.295329812 | loss | 0.623901094 | 0.328571282 | CDH22 | 37 | 20 |
| cg08401657 | 7.13E−10 | 3.03E−04 | −0.295308902 | 0.295308902 | loss | 0.672088802 | 0.3767799 | TFAP2A; TFAP2A; TFAP2A | 37 | 6 |
| cg10638827 | 7.13E−10 | 3.03E−04 | −0.295302052 | 0.295302052 | loss | 0.572592211 | 0.277290159 | MT1M | 37 | 16 |
| cg03546360 | 7.13E−10 | 3.03E−04 | −0.295279454 | 0.295279454 | loss | 0.47845303 | 0.183173576 | OHRS3 | 37 | 1 |
| cg16844333 | 7.13E−10 | 3.03E−04 | −0.295242297 | 0.295242297 | loss | 0.574627479 | 0.279385182 | LINC00864; LINC00864 | 37 | 10 |
| cg05112555 | 9.23E−10 | 3.92E−04 | −0.295237767 | 0.295237767 | loss | 0.719388943 | 0.424151176 | LOC100134391 | 37 | 17 |
| cg25075776 | 7.13E−10 | 3.03E−04 | −0.295236064 | 0.295236064 | loss | 0.866274023 | 0.571037959 | | 37 | 6 |
| cg09576209 | 7.13E−10 | 3.03E−04 | −0.295189578 | 0.295189578 | loss | 0.471295581 | 0.176106003 | CACNA1C; CACNA1C; CACNA1C; | 37 | 12 |
| | | | | | | | | CACNA1C; CACNA1C; | | |
| | | | | | | | | CACNA1C; CACNA1C; CACNA1C; | | |
| | | | | | | | | CACNA1C; CACNA1C; | | |
| | | | | | | | | CACNA1C; CACNA1C; CACNA1C; | | |
| | | | | | | | | CACNA1C; CACNA1C; | | |
| | | | | | | | | CACNA1C; CACNA1C; | | |
| | | | | | | | | CACNA1C; CACNA1C; CACNA1C; | | |
| | | | | | | | | CACNA1C; CACNA1C; | | |
| | | | | | | | | CACNA1C | | |
| cg05998295 | 1.73E−09 | 7.34E−04 | −0.295177767 | 0.295177767 | loss | 0.675652392 | 0.380474625 | XPO7 | 37 | 8 |
| cg14991769 | 7.13E−10 | 3.03E−04 | −0.295159817 | 0.295159817 | loss | 0.393551177 | 0.09839136 | TMEM176B; TMEM176B; | 37 | 7 |
| | | | | | | | | TMEM176B; TMEM176B; | | |
| | | | | | | | | TMEM176A | | |
| cg02605996 | 2.56E−09 | 1.09E−03 | −0.295146878 | 0.295146878 | loss | 0.71836986 | 0.423222982 | | 37 | 3 |
| cg05409945 | 7.13E−10 | 3.03E−04 | −0.295115639 | 0.295115639 | loss | 0.783694215 | 0.488578576 | C12orf80; LINC00592 | 37 | 12 |
| cg06201514 | 1.07E−07 | 4.55E−02 | −0.295106622 | 0.295106622 | loss | 0.669206981 | 0.374100359 | MYT1L | 37 | 2 |
| cg25944717 | 7.13E−10 | 3.03E−04 | −0.29509791 | 0.29509791 | loss | 0.654216145 | 0.359118235 | TRIM41; TRIM41; TRIM41 | 37 | 5 |
| cg02068015 | 7.13E−10 | 3.03E−04 | −0.295097591 | 0.295097591 | loss | 0.679726891 | 0.3846293 | | 37 | 22 |
| cg26006084 | 7.13E−10 | 3.03E−04 | −0.295097213 | 0.295097213 | loss | 0.971833625 | 0.676736412 | STK10 | 37 | 5 |
| cg09353289 | 7.77E−10 | 3.30E−04 | −0.295038089 | 0.295038089 | loss | 0.904836389 | 0.6097983 | | 37 | 10 |
| cg01020079 | 7.13E−10 | 3.03E−04 | −0.295031669 | 0.295031669 | loss | 0.789099492 | 0.494067824 | TMPRSS12 | 37 | 12 |
| cg02680050 | 7.13E−10 | 3.03E−04 | −0.294967044 | 0.294967044 | loss | 0.580044968 | 0.285077924 | LRCOL1 | 37 | 12 |
| cg05300029 | 2.35E−09 | 9.98E−04 | −0.294960965 | 0.294960965 | loss | 0.81680083 | 0.521839865 | | 37 | 14 |
| cg03684807 | 9.23E−10 | 3.92E−04 | −0.294926666 | 0.294926666 | loss | 0.706811777 | 0.411885112 | | 37 | 22 |
| cg25607670 | 7.13E−10 | 3.03E−04 | −0.294882106 | 0.294882106 | loss | 0.737018694 | 0.442136588 | MFI2; MF12 | 37 | 3 |
| cg23445321 | 1.19E−09 | 5.07E−04 | −0.294844697 | 0.294844697 | loss | 0.727407779 | 0.432563082 | SLC35F3 | 37 | 1 |
| cg10714061 | 7.13E−10 | 3.03E−04 | −0.294775506 | 0.294775506 | loss | 0.712151547 | 0.417376041 | IP6K3; IP6K3; IP6K3; IP6K3 | 37 | 6 |
| cg02996583 | 9.34E−10 | 3.97E−04 | −0.294774438 | 0.294774438 | loss | 0.799422567 | 0.504648129 | SLC45A4; SLC45A4; SLC45A4 | 37 | 8 |
| cg14769703 | 7.84E−10 | 3.33E−04 | −0.29476743 | 0.29476743 | loss | 0.445128185 | 0.150360755 | | 37 | 10 |
| cg27051129 | 7.13E−10 | 3.03E−04 | −0.294736775 | 0.294736775 | loss | 0.909644545 | 0.614907771 | DLEU7; DLEU7; DLEU7- | 37 | 13 |
| | | | | | | | | AS1 | | |
| cg23358886 | 7.13E−10 | 3.03E−04 | −0.294645037 | 0.294645037 | loss | 0.372181785 | 0.077536748 | RD3; RD3 | 37 | 1 |
| cg14135852 | 8.47E−10 | 3.60E−04 | −0.294634683 | 0.294634683 | loss | 0.94457743 | 0.649942747 | | 37 | X |
| cg19817820 | 7.13E−10 | 3.03E−04 | −0.294586036 | 0.294586036 | loss | 0.566403977 | 0.271817941 | KNDC1 | 37 | 10 |
| cg16228356 | 7.13E−10 | 3.03E−04 | −0.294493122 | 0.294493122 | loss | 0.493640206 | 0.199147084 | CRHR1 | 37 | 17 |
| cg02865928 | 7.13E−10 | 3.03E−04 | −0.294446691 | 0.294446691 | loss | 0.859755121 | 0.565308429 | | 37 | 17 |
| cg26298737 | 7.13E−10 | 3.03E−04 | −0.294434188 | 0.294434188 | loss | 0.565872047 | 0.271437859 | SRP9; SRP9 | 37 | 1 |
| cg24690094 | 3.14E−08 | 1.33E−02 | −0.294395131 | 0.294395131 | loss | 0.460405785 | 0.166010654 | DOC2GP | 37 | 11 |
| cg25423077 | 2.22E−09 | 9.41E−04 | −0.294371356 | 0.294371356 | loss | 0.722086021 | 0.427714665 | CDH20 | 37 | 18 |
| cg21211367 | 4.57E−09 | 1.94E−03 | 0.294363461 | 0.294363461 | gain | 0.150724456 | 0.445087918 | | 37 | 2 |
| cg00333946 | 1.30E−09 | 5.52E−04 | −0.294353238 | 0.294353238 | loss | 0.588542985 | 0.294189747 | | 37 | 15 |
| cg08632909 | 8.47E−10 | 3.60E−04 | −0.294310203 | 0.294310203 | loss | 0.618003068 | 0.323692865 | FADD | 37 | 11 |
| cg11426075 | 2.27E−09 | 9.66E−04 | −0.294284495 | 0.294284495 | loss | 0.414446418 | 0.120161923 | ERRFI1 | 37 | 1 |
| cg18025886 | 7.77E−10 | 3.30E−04 | −0.294263128 | 0.294263128 | loss | 0.532773087 | 0.238509959 | MFI2; MFI2 | 37 | 3 |
| cg00270625 | 3.97E−08 | 1.69E−02 | −0.294252618 | 0.294252618 | loss | 0.721538342 | 0.427285724 | CPT1B; CPT1B; CPT1B; CPT1B; | 37 | 22 |
| | | | | | | | | CPT1B; CPT1B; CPT1B; | | |
| | | | | | | | | CPT1B; CPT1B; CPT1B; CPT1B; | | |
| | | | | | | | | CPT1B; CPT1B; CHKB- | | |
| | | | | | | | | CPT1B | | |
| cg03122247 | 7.77E−10 | 3.30E−04 | −0.294241043 | 0.294241043 | loss | 0.751364113 | 0.457123071 | | 37 | 4 |
| cg22222281 | 7.13E−10 | 3.03E−04 | −0.294238862 | 0.294238862 | loss | 0.533651821 | 0.239412959 | CD9 | 37 | 12 |
| cg20785560 | 1.83E−09 | 7.75E−04 | −0.294229773 | 0.294229773 | loss | 0.433553881 | 0.139324108 | GIPC2 | 37 | 1 |
| cg12684052 | 7.13E−10 | 3.03E−04 | −0.294208055 | 0.294208055 | loss | 0.618959843 | 0.324751788 | | 37 | 17 |
| cg13016619 | 1.30E−09 | 5.52E−04 | −0.294186723 | 0.294186723 | loss | 0.921469564 | 0.627282841 | | 37 | 2 |
| cg00178928 | 7.13E−10 | 3.03E−04 | −0.294179548 | 0.294179548 | loss | 0.664598889 | 0.370419341 | | 37 | 2 |
| cg03569507 | 7.13E−10 | 3.03E−04 | −0.294169508 | 0.294169508 | loss | 0.693932455 | 0.399762947 | COPZ2 | 37 | 17 |
| cg06572093 | 7.13E−10 | 3.03E−04 | −0.294148675 | 0.294148675 | loss | 0.362113857 | 0.057965182 | | 37 | 14 |
| cg18046394 | 7.13E−10 | 3.03E−04 | −0.294116463 | 0.294116463 | loss | 0.531481881 | 0.237365418 | WIPF3 | 37 | 7 |
| cg09168805 | 4.29E−08 | 1.82E−02 | −0.294069141 | 0.294069141 | loss | 0.757343353 | 0.463274212 | ABCA4 | 37 | 1 |
| cg23907108 | 7.13E−10 | 3.03E−04 | −0.294000938 | 0.294000938 | loss | 0.535760391 | 0.241759453 | NRXN2; NRXN2; NRXN2 | 37 | 11 |
| cg15414745 | 6.80E−08 | 2.89E−02 | −0.293996346 | 0.293996346 | loss | 0.625917923 | 0.331921576 | DPPA5; DPPA5 | 37 | 6 |
| cg09798888 | 8.80E−09 | 3.74E−03 | −0.293988318 | 0.293988318 | loss | 0.35573148 | 0.061743162 | | 37 | 12 |
| cg16096531 | 7.13E−10 | 3.03E−04 | −0.29398016 | 0.29398016 | loss | 0.779826077 | 0.485845918 | SP7; SP7 | 37 | 12 |
| cg22360016 | 1.68E−09 | 7.12E−04 | −0.29390183 | 0.29390183 | loss | 0.450377379 | 0.156475549 | | 37 | 2 |
| cg04312620 | 7.13E−10 | 3.03E−04 | −0.293872293 | 0.293872293 | loss | 0.917204423 | 0.623332129 | SALL4; SALL4 | 37 | 20 |
| cg25974617 | 7.13E−10 | 3.03E−04 | −0.29380238 | 0.29380238 | loss | 0.552963321 | 0.259160941 | TRAM1; LOC286190 | 37 | 8 |
| cg09693926 | 4.21E−09 | 1.79E−03 | −0.293771378 | 0.293771378 | loss | 0.825235619 | 0.531464241 | ADAMTS2 | 37 | 5 |
| cg14818812 | 7.13E−10 | 3.03E−04 | −0.293646456 | 0.293646456 | loss | 0.786865162 | 0.493218706 | | 37 | 8 |
| cg16284953 | 7.13E−10 | 3.03E−04 | −0.293608206 | 0.293508206 | loss | 0.680020447 | 0.386412241 | | 37 | 8 |
| cg06042504 | 8.47E−10 | 3.60E−04 | −0.29359026 | 0.29359026 | loss | 0.825209719 | 0.531619459 | | 37 | 8 |
| cg25402787 | 7.13E−10 | 3.03E−04 | −0.293587328 | 0.293587328 | loss | 0.630719281 | 0.337131953 | | 37 | 15 |
| cg26164577 | 8.47E−10 | 3.60E−04 | −0.293544548 | 0.293544548 | loss | 0.439580817 | 0.146036269 | SNRPF | 37 | 12 |
| cg22160612 | 7.13E−10 | 3.03E−04 | −0.293518811 | 0.293518811 | loss | 0.441869928 | 0.148351118 | FAM118A; FAM118A | 37 | 22 |
| cg23052055 | 7.13E−10 | 3.03E−04 | −0.293495547 | 0.293495547 | loss | 0.522961906 | 0.229466359 | TP73; TP73; TP73; TP73; TP73; | 37 | 1 |
| | | | | | | | | TP73; TP73; TP73; TP73; | | |
| | | | | | | | | TP73; TP73; TP73 | | |
| cg19228034 | 7.13E−10 | 3.03E−04 | −0.293439128 | 0.293439128 | loss | 0.582928728 | 0.2894896 | | 37 | 7 |
| cg08818632 | 7.13E−10 | 3.03E−04 | −0.293430544 | 0.293430544 | loss | 0.769628668 | 0.476198124 | OTOP3; OTOP3 | 37 | 17 |
| cg04037970 | 1.30E−09 | 5.52E−04 | −0.29342721 | 0.29342721 | loss | 0.585016687 | 0.291589476 | | 37 | 7 |
| cg25903143 | 6.89E−09 | 2.93E−03 | −0.293418633 | 0.293418633 | loss | 0.848421215 | 0.555002582 | LOC338694 | 37 | 11 |
| cg20480274 | 9.23E−10 | 3.92E−04 | −0.2933856 | 0.2933856 | loss | 0.729801247 | 0.436415647 | SEZ6; SEZ6 | 37 | 17 |
| cg06588556 | 2.56E−09 | 1.09E−03 | −0.293366176 | 0.293366176 | loss | 0.676723653 | 0.383357476 | LDHAL6A; LDHAL6A | 37 | 11 |
| cg26537280 | 7.13E−10 | 3.03E−04 | −0.293363159 | 0.293363159 | loss | 0.454528647 | 0.161165488 | RWDD3; RWDD3; TMEM56- | 37 | 1 |
| | | | | | | | | RWDD3; RWDD3; RWDD3; | | |
| | | | | | | | | RWDD3; RWDD3; RWDD3 | | |
| cg06590170 | 7.13E−10 | 3.03E−04 | −0.293343643 | 0.293343643 | loss | 0.766391502 | 0.473047859 | | 37 | 6 |
| cg25502179 | 8.47E−10 | 3.60E−04 | −0.293303927 | 0.293303927 | loss | 0.951333021 | 0.658029094 | PRSS42 | 37 | 3 |
| cg21872908 | 7.13E−10 | 3.03E−04 | −0.293292689 | 0.293292689 | loss | 0.864208925 | 0.570916235 | C1orf177; C1orf177 | 37 | 1 |
| cg19645200 | 7.13E−10 | 3.03E−04 | −0.29328289 | 0.29328289 | loss | 0.836817949 | 0.543535059 | | 37 | 5 |
| cg00787996 | 1.81E−08 | 7.69E−03 | −0.293276752 | 0.293276752 | loss | 0.740808428 | 0.447531676 | TRIM31 | 37 | 6 |
| cg15138125 | 7.13E−10 | 3.03E−04 | −0.293256654 | 0.293256654 | loss | 0.376383377 | 0.083126723 | | 37 | 1 |
| cg13796250 | 3.57E−09 | 1.52E−03 | −0.293242846 | 0.293242846 | loss | 0.455611177 | 0.162368331 | | 37 | 6 |
| cg21209395 | 7.13E−10 | 3.03E−04 | −0.293126554 | 0.293126554 | loss | 0.488620083 | 0.195493529 | ATP8B5P; ATP8B5P | 37 | 9 |
| cg14995475 | 7.77E−10 | 3.30E−04 | −0.293079948 | 0.293079948 | loss | 0.899657825 | 0.606577876 | TNFSF9 | 37 | 19 |
| cg07115262 | 7.13E−10 | 3.03E−04 | −0.293056121 | 0.293056121 | loss | 0.758740726 | 0.465684606 | KRT86 | 37 | 12 |
| cg13970894 | 7.13E−10 | 3.03E−04 | −0.293011314 | 0.293011314 | loss | 0.869587349 | 0.576576035 | | 37 | 10 |
| cg10092257 | 7.13E−10 | 3.03E−04 | −0.29300946 | 0.29300946 | loss | 0.435317585 | 0.142308125 | | 37 | 18 |
| cg04624413 | 7.13E−10 | 3.03E−04 | −0.29300172 | 0.29300172 | loss | 0.708536979 | 0.415535259 | NKX6-3; NKX6-3 | 37 | 8 |
| cg26664752 | 2.45E−09 | 1.04E−03 | −0.29298573 | 0.29298573 | loss | 0.865283074 | 0.572297344 | | 37 | 11 |
| cg25879142 | 7.77E−10 | 3.30E−04 | −0.29295309 | 0.29295309 | loss | 0.412081542 | 0.119128451 | | 37 | 7 |
| cg09482093 | 7.13E−10 | 3.03E−04 | −0.292875376 | 0.292875376 | loss | 0.77663977 | 0.483764394 | RNF112 | 37 | 17 |
| cg03043822 | 1.68E−09 | 7.12E−04 | −0.292863276 | 0.292863276 | loss | 0.698085523 | 0.405222247 | PIWIL3; PIWIL3; TOP1P2; PIWIL3; | 37 | 22 |
| | | | | | | | | PIWIL3 | | |
| cg06978117 | 7.13E−10 | 3.03E−04 | −0.292806971 | 0.292806971 | loss | 0.713113383 | 0.420306412 | C11orf53 | 37 | 11 |
| cg13526469 | 7.77E−10 | 3.30E−04 | −0.292779264 | 0.292779264 | loss | 0.34927224 | 0.056492976 | | 37 | 3 |
| cg12291059 | 2.12E−08 | 9.01E−03 | −0.292761071 | 0.292761071 | loss | 0.842296289 | 0.549535218 | CTAGE1; CTAGE1 | 37 | 18 |
| cg22047282 | 7.13E−10 | 3.03E−04 | −0.292712769 | 0.292712769 | loss | 0.616822051 | 0.324109282 | EMILIN1 | 37 | 2 |
| cg02905426 | 7.13E−10 | 3.03E−04 | −0.292700855 | 0.292700855 | loss | 0.866156802 | 0.573455947 | FAM76A; FAM76A; FAM76A; | 37 | 1 |
| | | | | | | | | FAM76A; FAM76A | | |
| cg18713687 | 1.54E−08 | 6.56E−03 | −0.292686478 | 0.292686478 | loss | 0.837290366 | 0.544603888 | MUC4; MUC4; MUC4 | 37 | 3 |
| cg03533472 | 7.13E−10 | 3.03E−04 | −0.29265443 | 0.29265443 | loss | 0.513300915 | 0.220646485 | GPT2; GPT2 | 37 | 16 |
| cg23217153 | 7.13E−10 | 3.03E−04 | −0.292638718 | 0.292638718 | loss | 0.482176636 | 0.189537918 | | 37 | 2 |
| cg01477942 | 1.30E−09 | 5.52E−04 | −0.292626273 | 0.292626273 | loss | 0.949383496 | 0.656757224 | | 37 | 10 |
| cg19928597 | 5.39E−09 | 2.29E−03 | −0.292588006 | 0.292588006 | loss | 0.440537466 | 0.14794946 | | 37 | 7 |
| cg07130150 | 1.42E−09 | 6.01E−04 | −0.292586568 | 0.292586568 | loss | 0.663738709 | 0.371152141 | | 37 | 12 |
| cg18256205 | 1.73E−09 | 7.34E−04 | −0.292532104 | 0.292532104 | loss | 0.680140357 | 0.387608253 | | 37 | 5 |
| cg05351887 | 7.13E−10 | 3.03E−04 | −0.292494306 | 0.292494306 | loss | 0.38587746 | 0.093383155 | | 37 | 16 |
| cg01786973 | 7.13E−10 | 3.03E−04 | −0.292491825 | 0.292491825 | loss | 0.655355766 | 0.362863941 | NR5A1 | 37 | 9 |
| cg01693350 | 9.23E−10 | 3.92E−04 | −0.292471957 | 0.292471957 | loss | 0.521490987 | 0.229019029 | WT1; WT1; WT1; WT1; WT1; | 37 | 11 |
| | | | | | | | | WT1; WT1 | | |
| cg02152233 | 8.47E−10 | 3.60E−04 | −0.29242364 | 0.29242364 | loss | 0.886563675 | 0.594140035 | OPRD1 | 37 | 1 |
| cg14058476 | 7.84E−10 | 3.33E−04 | −0.2923669 | 0.2923669 | loss | 0.790430788 | 0.498063888 | NFATC4; NFATC4 | 37 | 14 |
| cg04589008 | 7.13E−10 | 3.03E−04 | −0.292333183 | 0.292333183 | loss | 0.867094425 | 0.574761241 | | 37 | 2 |
| cg04229928 | 2.40E−08 | 1.02E−02 | −0.29229035 | 0.29229035 | loss | 0.690203357 | 0.397913006 | USP2-AS1 | 37 | 11 |
| cg19193384 | 7.13E−10 | 3.03E−04 | −0.292284553 | 0.292284553 | loss | 0.419120787 | 0.126836234 | | 37 | 17 |
| cg04933168 | 8.47E−10 | 3.60E−04 | −0.292282928 | 0.292282928 | loss | 0.742247481 | 0.449964553 | CACNG2 | 37 | 22 |
| cg10783149 | 1.10E−09 | 4.65E−04 | −0.292199491 | 0.292199491 | loss | 0.841902491 | 0.549703 | | 37 | 14 |
| cg25362652 | 9.23E−10 | 3.92E−04 | −0.29217918 | 0.29217918 | loss | 0.496252115 | 0.204072935 | | 37 | 6 |
| cg21052104 | 7.13E−10 | 3.03E−04 | −0.292160466 | 0.292160466 | loss | 0.84492876 | 0.552768294 | ADAMTS2; ADAMTS2 | 37 | 5 |
| cg19774782 | 7.13E−10 | 3.03E−04 | −0.292101387 | 0.292101387 | loss | 0.848115281 | 0.556013894 | TACC2; TACC2 | 37 | 10 |
| cg14286514 | 7.13E−10 | 3.03E−04 | −0.29201517 | 0.29201517 | loss | 0.654854123 | 0.362838953 | DDX58 | 37 | 9 |
| cg16695380 | 7.13E−10 | 3.03E−04 | −0.291992152 | 0.291992152 | loss | 0.867241434 | 0.575249282 | MAD2L2 | 37 | 1 |
| cg13521842 | 7.13E−10 | 3.03E−04 | −0.291977009 | 0.291977009 | loss | 0.432943234 | 0.140966225 | BEGAIN; BEGAIN | 37 | 14 |
| cg12763978 | 7.13E−10 | 3.03E−04 | −0.291969511 | 0.291969511 | loss | 0.72348157 | 0.431512059 | | 37 | 6 |
| cg20740051 | 5.41E−08 | 2.30E−02 | −0.291962608 | 0.291962608 | loss | 0.885610702 | 0.593648094 | B4GALT3; B4GALT3; B4GALT3; | 37 | 1 |
| | | | | | | | | B4GALT3; B4GALT3; B4GALT3 | | |
| cg21784396 | 7.13E−10 | 3.03E−04 | −0.291946492 | 0.291946492 | loss | 0.36638734 | 0.074440848 | PRRT4; PRRT4 | 37 | 7 |
| cg14089881 | 6.30E−08 | 2.68E−02 | −0.291934448 | 0.291934448 | loss | 0.616938977 | 0.325004529 | ICMT; LINC00337 | 37 | 1 |
| cg24667603 | 3.40E−08 | 1.44E−02 | −0.291926828 | 0.291926828 | loss | 0.538347345 | 0.246420518 | | 37 | 15 |
| cg10394898 | 7.13E−10 | 3.03E−04 | −0.291907034 | 0.291907034 | loss | 0.566026723 | 0.274119688 | | 37 | 15 |
| cg12748639 | 5.07E−09 | 2.15E−03 | −0.291891471 | 0.291891471 | loss | 0.605487837 | 0.313596367 | CLEC4F; CLEC4F | 37 | 2 |
| cg04945076 | 1.01E−09 | 4.27E−04 | −0.291870157 | 0.291870157 | loss | 0.781407534 | 0.489537376 | | 37 | 12 |
| cg06979680 | 1.03E−08 | 4.39E−03 | −0.291851693 | 0.291851693 | loss | 0.91372744 | 0.621875747 | GRID1 | 37 | 10 |
| cg27529555 | 7.77E−10 | 3.30E−04 | −0.291822698 | 0.291822698 | loss | 0.588255204 | 0.296432506 | KLHDC8A; KLHDC8A; KLHDC8A; | 37 | 1 |
| | | | | | | | | KLHDC8A | | |
| cg14254433 | 9.23E−10 | 3.92E−04 | −0.291809649 | 0.291809649 | loss | 0.729387755 | 0.437578106 | PACSIN1; PACSIN1 | 37 | 6 |
| cg20761187 | 1.73E−09 | 7.34E−04 | −0.291802213 | 0.291802213 | loss | 0.675041619 | 0.383239406 | LOC100128239 | 37 | 11 |
| cg17818731 | 7.13E−10 | 3.03E−04 | −0.291797089 | 0.291797089 | loss | 0.786242942 | 0.494445853 | | 37 | 2 |
| cg23740940 | 8.47E−10 | 3.60E−04 | −0.291752176 | 0.291752176 | loss | 0.436563806 | 0.14481163 | LOC338694 | 37 | 11 |
| cg03209412 | 7.13E−10 | 3.03E−04 | −0.291718449 | 0.291718449 | loss | 0.49874316 | 0.207024712 | | 37 | 4 |
| cg09791746 | 2.35E−09 | 9.98E−04 | −0.291688386 | 0.291688386 | loss | 0.722458704 | 0.430770318 | ADAMTSLS | 37 | 19 |
| cg11060441 | 7.13E−10 | 3.03E−04 | −0.291636285 | 0.291636285 | loss | 0.845225079 | 0.553588794 | SSPO | 37 | 7 |
| cg12838168 | 7.13E−10 | 3.03E−04 | −0.291581716 | 0.291581716 | loss | 0.70963584 | 0.418054124 | LRRC15; LRRC15 | 37 | 3 |
| cg11541486 | 7.13E−10 | 3.03E−04 | −0.291567293 | 0.291567293 | loss | 0.376065598 | 0.084498305 | BTBD17; BTBD17 | 37 | 17 |
| cg17289378 | 7.84E−10 | 3.33E−04 | −0.291561907 | 0.291561907 | loss | 0.74133106 | 0.449769153 | | 37 | 6 |
| cg02855836 | 7.13E−10 | 3.03E−04 | −0.291552547 | 0.291552547 | loss | 0.534909058 | 0.243356512 | | 37 | 1 |
| cg27367526 | 8.47E−10 | 3.60E−04 | −0.291526469 | 0.291526469 | loss | 0.57847574 | 0.286949271 | STEAP2; STEAP2; STEAP2; STEAP2; | 37 | 7 |
| | | | | | | | | STEAP2; STEAP2 | | |
| cg24123198 | 1.32E−08 | 5.59E−03 | −0.291522441 | 0.291522441 | loss | 0.604409768 | 0.312887327 | | 37 | 4 |
| cg20128319 | 7.13E−10 | 3.03E−04 | −0.291461028 | 0.291461028 | loss | 0.656571387 | 0.365110359 | NEURL3 | 37 | 2 |
| cg14521421 | 3.02E−09 | 1.28E−03 | −0.291415356 | 0.291415356 | loss | 0.834964709 | 0.543549353 | ST5 | 37 | 11 |
| cg05671968 | 2.35E−09 | 9.98E−04 | −0.291340639 | 0.291340639 | loss | 0.880769051 | 0.589428412 | | 37 | 5 |
| cg27475169 | 7.13E−10 | 3.03E−04 | −0.291248927 | 0.291248927 | loss | 0.719998968 | 0.428750041 | | 37 | 11 |
| cg26656672 | 8.11E−09 | 3.44E−03 | −0.291212435 | 0.291212435 | loss | 0.900914811 | 0.609702376 | | 37 | 6 |
| cg04462774 | 7.77E−10 | 3.30E−04 | −0.291200625 | 0.291200625 | loss | 0.857849113 | 0.566648488 | AQP6 | 37 | 12 |
| cg18478731 | 7.13E−10 | 3.03E−04 | −0.291177376 | 0.291177376 | loss | 0.4287247 | 0.137547324 | | 37 | 2 |
| cg08828723 | 1.73E−09 | 7.34E−04 | −0.291151074 | 0.291151074 | loss | 0.88630903 | 0.595157956 | NOTCH4 | 37 | 6 |
| cg00933542 | 7.13E−10 | 3.03E−04 | −0.291144912 | 0.291144912 | loss | 0.7234328 | 0.432287888 | PCMT1; PCMT1; PCMT1; PCMT1; | 37 | 6 |
| | | | | | | | | PCMT1; PCMT1 | | |
| cg15325070 | 7.13E−10 | 3.03E−04 | −0.291128233 | 0.291128233 | loss | 0.774084321 | 0.482956088 | | 37 | 1 |
| cg23012917 | 9.23E−10 | 3.92E−04 | −0.291058406 | 0.291058406 | loss | 0.8494889 | 0.558430494 | | 37 | 1 |
| cg14502850 | 7.13E−10 | 3.03E−04 | −0.291047711 | 0.291047711 | loss | 0.783485064 | 0.492437353 | C1QL4 | 37 | 12 |
| cg01066936 | 7.13E−10 | 3.03E−04 | −0.291025318 | 0.291025318 | loss | 0.752040247 | 0.461014929 | TEF; TEF | 37 | 22 |
| cg23164850 | 7.13E−10 | 3.03E−04 | −0.291003486 | 0.291003486 | loss | 0.785755968 | 0.494752482 | FSCN2; FSCN2 | 37 | 17 |
| cg16180796 | 7.77E−10 | 3.30E−04 | −0.290993578 | 0.290993578 | loss | 0.37972883 | 0.088735252 | | 37 | 7 |
| cg13930544 | 6.89E−09 | 2.93E−03 | −0.290841601 | 0.290841601 | loss | 0.795341925 | 0.504500324 | GCSAML; GCSAML; GCSAML; | 37 | 1 |
| | | | | | | | | GCSAML; GCSAML; GCSAML; | | |
| | | | | | | | | OR2C3; GCSAML- | | |
| | | | | | | | | AS1; GCSAML-AS1 | | |
| cg09933329 | 7.13E−10 | 3.03E−04 | −0.290834742 | 0.290834742 | loss | 0.556241342 | 0.2654056 | GCK; GCK; GCK | 37 | 7 |
| cg08817693 | 1.11E−09 | 4.72E−04 | −0.290824066 | 0.290824066 | loss | 0.705676742 | 0.414852676 | C12orf23 | 37 | 12 |
| cg00927699 | 6.87E−09 | 2.92E−03 | −0.290811409 | 0.290811409 | loss | 0.850130615 | 0.559319206 | ADARB1; ADARB1; ADARB1; | 37 | 21 |
| | | | | | | | | ADARB1; SSR4P1; SSR4P1; | | |
| | | | | | | | | ADARB1; ADARB1; ADARB1; | | |
| | | | | | | | | ADARB1 | | |
| cg17100943 | 7.13E−10 | 3.03E−04 | −0.290802811 | 0.290802811 | loss | 0.46404644 | 0.173243628 | SLC25A23 | 37 | 19 |
| cg00639837 | 1.01E−09 | 4.27E−04 | −0.290756469 | 0.290756469 | loss | 0.901557287 | 0.610800818 | | 37 | 13 |
| cg02773436 | 7.13E−10 | 3.03E−04 | −0.290648363 | 0.290648363 | loss | 0.545764387 | 0.255116024 | | 37 | 10 |
| cg19027710 | 8.47E−10 | 3.60E−04 | −0.290624201 | 0.290624201 | loss | 0.901699283 | 0.611075082 | CHST2 | 37 | 3 |
| cg23791325 | 2.56E−09 | 1.09E−03 | −0.290600825 | 0.290600825 | loss | 0.72222616 | 0.431625335 | | 37 | 2 |
| cg18163342 | 7.13E−10 | 3.03E−04 | −0.290559681 | 0.290559681 | loss | 0.583347923 | 0.292788241 | CEP170B; CEP170B | 37 | 14 |
| cg20292791 | 1.00E−07 | 4.25E−02 | −0.290495313 | 0.290495313 | loss | 0.819057631 | 0.528562318 | DAB1 | 37 | 1 |
| cg24996814 | 2.35E−09 | 9.98E−04 | −0.290489986 | 0.290489986 | loss | 0.606665304 | 0.316175318 | | 37 | 11 |
| cg06282802 | 7.13E−10 | 3.03E−04 | −0.290468866 | 0.290468866 | loss | 0.71229856 | 0.421829694 | | 37 | 2 |
| cg05589489 | 1.87E−09 | 7.93E−04 | −0.290435973 | 0.290435973 | loss | 0.71277509 | 0.422339118 | | 37 | 12 |
| cg09084279 | 7.13E−10 | 3.03E−04 | −0.29038898 | 0.29038898 | loss | 0.866351551 | 0.575962571 | FSCN2; FSCN2 | 37 | 17 |
| cg01801826 | 7.13E−10 | 3.03E−04 | −0.290380031 | 0.290380031 | loss | 0.536472755 | 0.246092724 | | 37 | 8 |
| cg03001867 | 7.13E−10 | 3.03E−04 | −0.290341224 | 0.290341224 | loss | 0.936222936 | 0.645881712 | | 37 | 16 |
| cg20790798 | 7.48E−09 | 3.18E−03 | −0.290323233 | 0.290323233 | loss | 0.676184304 | 0.385861071 | | 37 | 5 |
| cg02899903 | 7.13E−10 | 3.03E−04 | −0.290307961 | 0.290307961 | loss | 0.822996702 | 0.532688741 | PITRM1; PITRM1; PITRM1 | 37 | 10 |
| cg11872743 | 1.83E−09 | 7.75E−04 | −0.290299839 | 0.290299839 | loss | 0.572182245 | 0.281882406 | | 37 | 17 |
| cg25599129 | 8.47E−10 | 3.60E−04 | −0.290289198 | 0.290289198 | loss | 0.501601351 | 0.211312153 | | 37 | 1 |
| cg16885093 | 7.13E−10 | 3.03E−04 | −0.290266568 | 0.290266568 | loss | 0.952304926 | 0.662038359 | CSHL1; CSHL1; CSHL1; CSHL1 | 37 | 17 |
| cg23388085 | 1.01E−09 | 4.27E−04 | −0.290235942 | 0.290235942 | loss | 0.943771925 | 0.653535982 | | 37 | 6 |
| cg15197065 | 7.77E−10 | 3.30E−04 | −0.290216646 | 0.290216646 | loss | 0.636234515 | 0.346017869 | CMYA5 | 37 | 5 |
| cg02940147 | 7.13E−10 | 3.03E−04 | −0.290199637 | 0.290199637 | loss | 0.467168345 | 0.176968708 | ROBO3 | 37 | 11 |
| cg10770023 | 1.68E−09 | 7.12E−04 | −0.290181206 | 0.290181206 | loss | 0.499324742 | 0.209143535 | CPT1B; CPT1B; CPT1B; CPT1B; | 37 | 22 |
| | | | | | | | | CPT1B; CPT1B; CPT1B; | | |
| | | | | | | | | CPT1B; CHKB-CPT1B | | |
| cg20171429 | 1.42E−09 | 6.01E−04 | −0.290158255 | 0.290158255 | loss | 0.513885096 | 0.223726841 | C7orf63; C7orf63 | 37 | 7 |
| cg18038544 | 1.89E−09 | 8.01E−04 | −0.29007342 | 0.29007342 | loss | 0.548556988 | 0.258483569 | GRIP2 | 37 | 3 |
| cg23688479 | 7.13E−10 | 3.03E−04 | −0.290010314 | 0.290010314 | loss | 0.696662438 | 0.406652124 | CCDC152 | 37 | 5 |
| cg05877550 | 1.68E−09 | 7.12E−04 | −0.289978101 | 0.289978101 | loss | 0.640063719 | 0.350085618 | | 37 | 16 |
| cg18201671 | 7.13E−10 | 3.03E−04 | −0.289942921 | 0.289942921 | loss | 0.588183504 | 0.298240582 | TLR5 | 37 | 1 |
| cg05180717 | 7.77E−10 | 3.30E−04 | −0.289903103 | 0.289903103 | loss | 0.510009862 | 0.220106759 | EMC4; EMC4 | 37 | 15 |
| cg14076971 | 7.13E−10 | 3.03E−04 | −0.289899193 | 0.289899193 | loss | 0.853632328 | 0.563733135 | C1orf86; C1orf86 | 37 | 1 |
| cg12743470 | 7.13E−10 | 3.03E−04 | −0.289889451 | 0.289889451 | loss | 0.685911298 | 0.396021847 | VRTN | 37 | 14 |
| cg15015340 | 7.13E−10 | 3.03E−04 | −0.289872164 | 0.289872164 | loss | 0.470728398 | 0.180856234 | DAGLA | 37 | 11 |
| cg13023646 | 8.47E−10 | 3.60E−04 | −0.289870729 | 0.289870729 | loss | 0.370217351 | 0.080346622 | TRIM61; TRIM61 | 37 | 4 |
| cg05467458 | 7.77E−10 | 3.30E−04 | −0.289859295 | 0.289859295 | loss | 0.71790933 | 0.428050035 | SLC7A9; SLC7A9; SLC7A9 | 37 | 19 |
| cg23098371 | 7.13E−10 | 3.03E−04 | −0.289811509 | 0.289811509 | loss | 0.913561326 | 0.623749818 | | 37 | 1 |
| cg17628249 | 7.13E−10 | 3.03E−04 | −0.289798135 | 0.289798135 | loss | 0.650155206 | 0.360357071 | C17orf64 | 37 | 17 |
| cg02616418 | 1.68E−09 | 7.12E−04 | −0.2897925 | 0.2897925 | loss | 0.789730342 | 0.499937841 | TRIM2; TRIM2 | 37 | 4 |
| cg11400650 | 7.13E−10 | 3.03E−04 | −0.289792072 | 0.289792072 | loss | 0.699281613 | 0.409489541 | NOX5; NOX5; NOX5; MIR548H4; | 37 | 15 |
| | | | | | | | | NOX5; NOX5 | | |
| cg06950634 | 7.13E−10 | 3.03E−04 | −0.28974873 | 0.28974873 | loss | 0.947893047 | 0.658144318 | | 37 | 11 |
| cg13639937 | 7.13E−10 | 3.03E−04 | −0.2897014 | 0.2897014 | loss | 0.885972111 | 0.596270712 | | 37 | 1 |
| cg00782200 | 4.57E−09 | 1.94E−03 | −0.289677531 | 0.289677531 | loss | 0.395268319 | 0.105590788 | | 37 | 2 |
| cg15780361 | 7.13E−10 | 3.03E−04 | −0.289644876 | 0.289644876 | loss | 0.665837664 | 0.376192788 | ALS2CR11; ALS2CR11; ALS2CR11; | 37 | 2 |
| | | | | | | | | ALS2CR11; ALS2CR11; | | |
| | | | | | | | | ALS2CR11; ALS2CR11; | | |
| | | | | | | | | ALS2CR11 | | |
| cg14531437 | 9.23E−10 | 3.92E−04 | −0.28964082 | 0.28964082 | loss | 0.854083785 | 0.564442965 | | 37 | 9 |
| cg17804348 | 7.13E−10 | 3.03E−04 | −0.289632127 | 0.289632127 | loss | 0.373111811 | 0.083479685 | TP73; TP73; TP73; TP73; TP73; | 37 | 1 |
| | | | | | | | | TP73; TP73; TP73; TP73; | | |
| | | | | | | | | TP73; TP73; TP73; TP73 | | |
| cg00493304 | 7.77E−10 | 3.30E−04 | −0.289601153 | 0.289601153 | loss | 0.889497758 | 0.599896606 | GTF2A1L; STON1- | 37 | 2 |
| | | | | | | | | GTF2A1L; STON1- | | |
| | | | | | | | | GTF2A1L; GTF2A1L; STON1- | | |
| | | | | | | | | GTF2A1L | | |
| cg22897224 | 4.57E−09 | 1.94E−03 | −0.289570671 | 0.289570671 | loss | 0.736078347 | 0.446507676 | PIWIL3; PIWIL3; TOP1P2; PIWIL3; | 37 | 22 |
| | | | | | | | | PIWIL3 | | |
| cg23009263 | 7.13E−10 | 3.03E−04 | −0.289543047 | 0.289543047 | loss | 0.854328864 | 0.564785818 | | 37 | 14 |
| cg10053622 | 3.88E−09 | 1.65E−03 | −0.289484774 | 0.289484774 | loss | 0.777050962 | 0.487566188 | DNAH2 | 37 | 17 |
| cg00412675 | 1.54E−09 | 6.54E−04 | −0.289436169 | 0.289436169 | loss | 0.786178098 | 0.496741929 | GATA4 | 37 | 8 |
| cg06642945 | 5.39E−09 | 2.29E−03 | −0.289416769 | 0.289416769 | loss | 0.848148398 | 0.558731629 | PACRG; PACRG; PACRG | 37 | 6 |
| cg01924435 | 7.13E−10 | 3.03E−04 | −0.289340039 | 0.289340039 | loss | 0.41404984 | 0.124709801 | | 37 | 4 |
| cg08289350 | 7.13E−10 | 3.03E−04 | −0.289338297 | 0.289338297 | loss | 0.539353879 | 0.250015582 | CYGB; PRCD | 37 | 17 |
| cg20949251 | 8.47E−10 | 3.60E−04 | −0.28927382 | 0.28927382 | loss | 0.858957432 | 0.569683612 | | 37 | 6 |
| cg05474265 | 7.13E−10 | 3.03E−04 | −0.289202722 | 0.289202722 | loss | 0.595809681 | 0.306606959 | | 37 | 4 |
| cg03309770 | 7.13E−10 | 3.03E−04 | −0.289151724 | 0.289151724 | loss | 0.391379053 | 0.102227329 | TVP23A; TVP23A | 37 | 16 |
| cg14333454 | 7.13E−10 | 3.03E−04 | −0.28915099 | 0.28915099 | loss | 0.681995408 | 0.392844418 | SFN | 37 | 1 |
| cg02930419 | 8.47E−10 | 3.60E−04 | −0.289132599 | 0.289132599 | loss | 0.413668343 | 0.124535745 | | 37 | 17 |
| cg06221946 | 7.13E−10 | 3.03E−04 | −0.2890655 | 0.2890655 | loss | 0.636896311 | 0.347830812 | MARCKSL1; MARCKSL1 | 37 | 1 |
| cg16876823 | 7.77E−10 | 3.30E−04 | −0.289060533 | 0.289060533 | loss | 0.662461221 | 0.373400688 | TLE6; TLE6 | 37 | 19 |
| cg15974931 | 2.12E−08 | 9.01E−03 | −0.289023303 | 0.289023303 | loss | 0.843270032 | 0.554246729 | PKHD1L1 | 37 | 8 |
| cg24557048 | 9.23E−10 | 3.92E−04 | −0.288998252 | 0.288998252 | loss | 0.496728481 | 0.207730229 | PRKCZ; PRKCZ; PRKCZ; PRKCZ | 37 | 1 |
| cg02064284 | 7.13E−10 | 3.03E−04 | −0.28897856 | 0.28897856 | loss | 0.390442194 | 0.101463634 | | 37 | 14 |
| cg07626700 | 7.13E−10 | 3.03E−04 | −0.288947721 | 0.288947721 | loss | 0.611807462 | 0.322859741 | NOM1 | 37 | 7 |
| cg16442587 | 7.13E−10 | 3.03E−04 | −0.288822206 | 0.288822206 | loss | 0.856385242 | 0.567563035 | | 37 | 18 |
| cg19865237 | 8.65E−10 | 3.67E−04 | −0.288807357 | 0.288807357 | loss | 0.56493951 | 0.276132153 | | 37 | 14 |
| cg11855943 | 7.13E−10 | 3.03E−04 | −0.288728354 | 0.288728354 | loss | 0.525956077 | 0.237227724 | | 37 | 17 |
| cg02479842 | 1.99E−09 | 8.43E−04 | −0.288703579 | 0.288703579 | loss | 0.856499832 | 0.567796253 | | 37 | 2 |
| cg12723492 | 8.47E−10 | 3.60E−04 | −0.288701457 | 0.288701457 | loss | 0.706233804 | 0.417532347 | KIRREL3; KIRREL3 | 37 | 11 |
| cg16454316 | 8.47E−10 | 3.60E−04 | −0.288689861 | 0.288689861 | loss | 0.718788938 | 0.430099076 | | 37 | 6 |
| cg22937320 | 7.13E−10 | 3.03E−04 | −0.288682392 | 0.288682392 | loss | 0.822640157 | 0.533957765 | FAM154A | 37 | 9 |
| cg20262330 | 7.13E−10 | 3.03E−04 | −0.288634216 | 0.288634216 | loss | 0.501808951 | 0.213174735 | SYNJ2BP-COX16; SYNJ2BP- | 37 | 14 |
| | | | | | | | | COX16; SYNJ2BP- | | |
| | | | | | | | | COX16; COX16; COX16 | | |
| cg08572513 | 2.12E−08 | 9.01E−03 | −0.288597789 | 0.288597789 | loss | 0.746348619 | 0.457750829 | KIRREL3; KIRREL3 | 37 | 11 |
| cg03382250 | 7.13E−10 | 3.03E−04 | −0.288593013 | 0.288593013 | loss | 0.55978903 | 0.271196018 | KIRREL3; KIRREL3 | 37 | 11 |
| cg05316463 | 7.13E−10 | 3.03E−04 | −0.288575122 | 0.288575122 | loss | 0.393947074 | 0.105371951 | CLMN | 37 | 14 |
| cg22928954 | 7.13E−10 | 3.03E−04 | −0.288509503 | 0.288509503 | loss | 0.502261013 | 0.213751511 | | 37 | 19 |
| cg17545182 | 7.48E−09 | 3.18E−03 | −0.288470204 | 0.288470204 | loss | 0.816374145 | 0.527903941 | GCSAML; GCSAML; GCSAML; | 37 | 1 |
| | | | | | | | | GCSAML; GCSAML; GCSAML; | | |
| | | | | | | | | OR2C3; GCSAML- | | |
| | | | | | | | | AS1; GCSAML-AS1 | | |
| cg25104872 | 7.13E−10 | 3.03E−04 | −0.288384017 | 0.288384017 | loss | 0.69741337 | 0.409029353 | LOC401242 | 37 | 6 |
| cg10962524 | 3.14E−08 | 1.33E−02 | −0.288346559 | 0.288346559 | loss | 0.674479489 | 0.386132929 | HAPLN1; HAPLN1 | 37 | 5 |
| cg11709544 | 7.13E−10 | 3.03E−04 | −0.288341336 | 0.288341336 | loss | 0.364966785 | 0.076625449 | STMN1; STMN1; STMN1; STMN1; | 37 | 1 |
| | | | | | | | | STMN1; MIR3917 | | |
| cg09893305 | 1.54E−08 | 6.56E−03 | −0.288333201 | 0.288333201 | loss | 0.736159836 | 0.447826635 | HAPLN1 | 37 | 5 |
| cg18335290 | 7.77E−10 | 3.30E−04 | −0.288267713 | 0.288267713 | loss | 0.498257077 | 0.209989365 | LIMS2 | 37 | 2 |
| cg12912758 | 2.78E−09 | 1.18E−03 | −0.288094902 | 0.288094902 | loss | 0.749650402 | 0.4615555 | | 37 | 6 |
| cg05063670 | 8.80E−09 | 3.74E−03 | −0.28800739 | 0.28800739 | loss | 0.831349966 | 0.543342576 | RPS6KA2; RPS6KA2 | 37 | 6 |
| cg03142554 | 7.77E−10 | 3.30E−04 | −0.287995667 | 0.287995667 | loss | 0.703845249 | 0.415849582 | IBTK | 37 | 6 |
| cg08370173 | 8.47E−10 | 3.60E−04 | −0.287977067 | 0.287977067 | loss | 0.754324902 | 0.466347835 | | 37 | 17 |
| cg23549902 | 6.30E−08 | 2.68E−02 | −0.287959007 | 0.287959007 | loss | 0.51925217 | 0.231293163 | ZNF890P; ZNF890P | 37 | 7 |
| cg03800065 | 7.13E−10 | 3.03E−04 | −0.287906187 | 0.287906187 | loss | 0.72120897 | 0.433302782 | | 37 | 10 |
| cg03880033 | 8.11E−09 | 3.44E−03 | −0.287904808 | 0.287904808 | loss | 0.660058049 | 0.372153241 | CES1; CES1; CES1 | 37 | 16 |
| cg14836522 | 7.13E−10 | 3.03E−04 | −0.287899641 | 0.287899641 | loss | 0.5682748 | 0.280375159 | SDC3 | 37 | 1 |
| cg21193577 | 7.13E−10 | 3.03E−04 | −0.287860725 | 0.287860725 | loss | 0.549997402 | 0.262136676 | BEGAIN; BEGAIN | 37 | 14 |
| cg10982664 | 7.13E−10 | 3.03E−04 | −0.28784792 | 0.28784792 | loss | 0.776250749 | 0.488402829 | BHMT | 37 | 5 |
| cg16074228 | 7.13E−10 | 3.03E−04 | −0.287823588 | 0.287823588 | loss | 0.8634718 | 0.575648212 | KHDC3L | 37 | 6 |
| cg27179680 | 1.01E−09 | 4.27E−04 | −0.287822771 | 0.287822771 | loss | 0.747633542 | 0.459810771 | | 37 | 1 |
| cg23052855 | 9.54E−09 | 4.05E−03 | −0.287812242 | 0.287812242 | loss | 0.640491083 | 0.352678841 | ZDHHC19 | 37 | 3 |
| cg04017512 | 4.97E−09 | 2.11E−03 | −0.287803975 | 0.287803975 | loss | 0.701894887 | 0.414090912 | | 37 | 10 |
| cg01968133 | 1.19E−09 | 5.07E−04 | −0.287802923 | 0.287802923 | loss | 0.860116817 | 0.572313894 | CASZ1; CASZ1 | 37 | 1 |
| cg00658082 | 7.13E−10 | 3.03E−04 | −0.287767222 | 0.287767222 | loss | 0.471102381 | 0.183335159 | RTKN | 37 | 2 |
| cg02978140 | 7.13E−10 | 3.03E−04 | −0.287734139 | 0.287734139 | loss | 0.518406751 | 0.230672612 | | 37 | 1 |
| cg04342483 | 2.78E−09 | 1.18E−03 | −0.287727138 | 0.287727138 | loss | 0.629544891 | 0.341817753 | CAMTA1 | 37 | 1 |
| cg08548500 | 7.13E−10 | 3.03E−04 | −0.28771649 | 0.28771649 | loss | 0.763146208 | 0.475429718 | F10 | 37 | 13 |
| cg08159989 | 9.22E−08 | 3.91E−02 | −0.287688258 | 0.287688258 | loss | 0.910373711 | 0.622685453 | KLHDC8A; KLHDC8A; KLHDC8A; | 37 | 1 |
| | | | | | | | | KLHDC8A; KLHDC8A; | | |
| | | | | | | | | KLHDC8A; KLHDC8A; KLHDC8A | | |
| cg15246511 | 1.68E−09 | 7.12E−04 | −0.287672571 | 0.287672571 | loss | 0.729215647 | 0.441543076 | APOB | 37 | 2 |
| cg04140525 | 7.13E−10 | 3.03E−04 | −0.287662029 | 0.257662029 | loss | 0.796677706 | 0.509015676 | | 37 | 1 |
| cg16428357 | 1.68E−09 | 7.12E−04 | −0.287591043 | 0.287591043 | loss | 0.820974343 | 0.5333833 | | 37 | 20 |
| cg07013104 | 7.13E−10 | 3.03E−04 | −0.287566718 | 0.287566718 | loss | 0.548582 | 0.261015282 | PLEKHB1; PLEKHB1; PLEKHB1; | 37 | 11 |
| | | | | | | | | PLEKKB1 | | |
| cg03883572 | 7.13E−10 | 3.03E−04 | −0.287512825 | 0.287512825 | loss | 0.507201319 | 0.219688494 | MAST4; MAST4 | 37 | 5 |
| cg25924688 | 7.13E−10 | 3.03E−04 | −0.287500233 | 0.287500233 | loss | 0.489777315 | 0.202277082 | BBS4; BBS4; HIGD2B; BBS4; | 37 | 15 |
| | | | | | | | | BBS4 | | |
| cg25396728 | 2.30E−08 | 9.75E−03 | −0.287499073 | 0.287499073 | loss | 0.429774709 | 0.142275636 | IQSEC3; IQSEC3; LOC574538 | 37 | 12 |
| cg18941641 | 2.35E−09 | 9.98E−04 | −0.287490905 | 0.287490905 | loss | 0.459942615 | 0.172451711 | | 37 | 10 |
| cg07689920 | 7.13E−10 | 3.03E−04 | −0.287482962 | 0.287482962 | loss | 0.666573657 | 0.379090694 | PLEKHB1; PLEKHB1 | 37 | 11 |
| cg00398420 | 7.13E−10 | 3.03E−04 | −0.287460695 | 0.287460695 | loss | 0.427626119 | 0.140165424 | | 37 | 16 |
| cg14962509 | 7.13E−10 | 3.03E−04 | −0.287390082 | 0.287390082 | loss | 0.945922906 | 0.658532824 | TFAP2E | 37 | 1 |
| cg05017199 | 7.13E−10 | 3.03E−04 | −0.287366703 | 0.287366703 | loss | 0.400201455 | 0.112834752 | TMEM176B; TMEM176B; | 37 | 7 |
| | | | | | | | | TMEM176B; TMEM176B; | | |
| | | | | | | | | TMEM176A | | |
| cg14958041 | 8.47E−10 | 3.60E−04 | −0.287327942 | 0.287327942 | loss | 0.734124425 | 0.446796482 | | 37 | 9 |
| cg16844661 | 7.13E−10 | 3.03E−04 | −0.287226112 | 0.287226112 | loss | 0.761584942 | 0.474358829 | TH; TH; TH | 37 | 11 |
| cg15774065 | 7.13E−10 | 3.03E−04 | −0.287220689 | 0.287220689 | loss | 0.701931472 | 0.414710782 | FSCN2; FSCN2 | 37 | 17 |
| cg02831900 | 7.13E−10 | 3.03E−04 | −0.28721558 | 0.28721558 | loss | 0.943750668 | 0.656535088 | | 37 | 4 |
| cg10807643 | 3.88E−09 | 1.65E−03 | −0.287197615 | 0.287197615 | loss | 0.679060209 | 0.391862594 | TBPL2; TBPL2 | 37 | 14 |
| cg08288433 | 1.30E−09 | 5.52E−04 | −0.287145916 | 0.287145916 | loss | 0.730774987 | 0.443629071 | CCDC144NL; CCDC144NL; | 37 | 17 |
| | | | | | | | | LOC440416 | | |
| cg02769477 | 1.07E−07 | 4.55E−02 | −0.287135173 | 0.287135173 | loss | 0.652967475 | 0.365832302 | | 37 | 1 |
| cg18288532 | 9.23E−10 | 3.92E−04 | −0.28712964 | 0.28712964 | loss | 0.446742187 | 0.159612547 | | 37 | 6 |
| cg02174341 | 7.13E−10 | 3.03E−04 | −0.287087401 | 0.287087401 | loss | 0.655177577 | 0.368090176 | UCMA; UCMA | 37 | 10 |
| cg03579045 | 2.78E−09 | 1.18E−03 | −0.287065175 | 0.287065175 | loss | 0.773810887 | 0.486745712 | GATA4 | 37 | 8 |
| cg07779699 | 7.13E−10 | 3.03E−04 | −0.286980782 | 0.286980782 | loss | 0.876024564 | 0.589043782 | FAM71E2 | 37 | 19 |
| cg11334165 | 7.13E−10 | 3.03E−04 | −0.286974464 | 0.286974464 | loss | 0.460933623 | 0.173959159 | | 37 | 5 |
| cg24635754 | 1.01E−09 | 4.27E−04 | −0.286946777 | 0.286946777 | loss | 0.885318642 | 0.598371865 | | 37 | 1 |
| cg01027976 | 7.13E−10 | 3.03E−04 | −0.286941771 | 0.286941771 | loss | 0.731610542 | 0.444668771 | | 37 | 22 |
| cg18660345 | 7.77E−10 | 3.30E−04 | −0.28690712 | 0.28690712 | loss | 0.443265992 | 0.156358872 | MYL9; MYL9 | 37 | 20 |
| cg14485097 | 7.13E−10 | 3.03E−04 | −0.286895538 | 0.286895538 | loss | 0.456069432 | 0.169173894 | | 37 | 7 |
| cg08878450 | 7.13E−10 | 3.03E−04 | −0.286864783 | 0.286864783 | loss | 0.46709367 | 0.180228887 | OSCP1; OSCP1 | 37 | 1 |
| cg27168291 | 7.13E−10 | 3.03E−04 | −0.286852948 | 0.286852948 | loss | 0.884975425 | 0.598122476 | ZBTB22; TAPBP; ZBTB22; TAPBP; | 37 | 6 |
| | | | | | | | | TAPBP | | |
| cg17520215 | 7.77E−10 | 3.30E−04 | −0.286788707 | 0.286788707 | loss | 0.698699077 | 0.411910371 | GAS2L3 | 37 | 12 |
| cg00314115 | 1.10E−09 | 4.65E−04 | −0.28678276 | 0.28678276 | loss | 0.52363156 | 0.2368488 | | 37 | 2 |
| cg05373875 | 2.35E−09 | 9.98E−04 | −0.286729589 | 0.286729589 | loss | 0.810870842 | 0.524141253 | | 37 | 20 |
| cg05369327 | 7.13E−10 | 3.03E−04 | −0.286725682 | 0.286725682 | loss | 0.332768813 | 0.046043131 | | 37 | 14 |
| cg16677399 | 1.30E−09 | 5.52E−04 | −0.286659328 | 0.286659328 | loss | 0.848564328 | 0.561905 | LOC401242 | 37 | 6 |
| cg03302546 | 7.13E−10 | 3.03E−04 | −0.286610058 | 0.286610058 | loss | 0.850157128 | 0.563547071 | | 37 | 13 |
| cg10359157 | 7.84E−10 | 3.33E−04 | −0.28657761 | 0.28657761 | loss | 0.873234569 | 0.586656959 | DLEU7; DLEU7; DLEU7- | 37 | 13 |
| | | | | | | | | AS1 | | |
| cg27128941 | 7.13E−10 | 3.03E−04 | −0.286508087 | 0.286508087 | loss | 0.64729224 | 0.360784153 | | 37 | 5 |
| cg10519417 | 7.13E−10 | 3.03E−04 | −0.286485599 | 0.286485599 | loss | 0.487317164 | 0.200831565 | | 37 | 17 |
| cg12606933 | 7.13E−10 | 3.03E−04 | −0.286436795 | 0.286436795 | loss | 0.727267277 | 0.440830482 | SOX30; SOX30 | 37 | 5 |
| cg18498622 | 7.48E−09 | 3.18E−03 | −0.286421945 | 0.286421945 | loss | 0.632548804 | 0.346126859 | GABRQ | 37 | X |
| cg06993467 | 7.13E−10 | 3.03E−04 | −0.286416237 | 0.286416237 | loss | 0.968236208 | 0.681819971 | RAP1GAP; RAP1GAP; RAP1GAP | 37 | 1 |
| cg11966998 | 7.77E−10 | 3.30E−04 | −0.286357146 | 0.286357146 | loss | 0.475173834 | 0.188816688 | | 37 | 11 |
| cg19520234 | 4.97E−09 | 2.11E−03 | −0.286335281 | 0.286335281 | loss | 0.670469692 | 0.384134412 | LOC152586 | 37 | 4 |
| cg03012289 | 3.88E−09 | 1.65E−03 | −0.286295257 | 0.286295257 | loss | 0.881790245 | 0.595494988 | PKHD1L1 | 37 | 8 |
| cg13731640 | 2.16E−09 | 9.17E−04 | −0.286287218 | 0.286287218 | loss | 0.609386783 | 0.323099565 | | 37 | 7 |
| cg00907008 | 1.68E−09 | 7.12E−04 | −0.286250122 | 0.286250122 | loss | 0.710055381 | 0.423805259 | CHL1; CHL1 | 37 | 3 |
| cg26607337 | 7.13E−10 | 3.03E−04 | −0.286235589 | 0.286235589 | loss | 0.527306189 | 0.2410706 | | 37 | 5 |
| cg03549506 | 7.13E−10 | 3.03E−04 | −0.286197428 | 0.286197428 | loss | 0.795277581 | 0.509080153 | | 37 | 10 |
| cg25005368 | 7.84E−10 | 3.33E−04 | −0.28613698 | 0.28613698 | loss | 0.426709577 | 0.140572597 | | 37 | 10 |
| cg23008871 | 7.13E−10 | 3.03E−04 | −0.286123 | 0.286123 | loss | 0.341228885 | 0.055105885 | | 37 | 17 |
| cg06915226 | 7.77E−10 | 3.30E−04 | −0.286075193 | 0.286075193 | loss | 0.81935587 | 0.533280676 | ADD2; ADD2; ADD2; ADD2; | 37 | 2 |
| | | | | | | | | ADD2 | | |
| cg21849552 | 7.13E−10 | 3.03E−04 | −0.286070867 | 0.286070867 | loss | 0.541371391 | 0.255300524 | | 37 | 3 |
| cg24202119 | 1.22E−08 | 5.16E−03 | −0.286023118 | 0.286023118 | loss | 0.71679513 | 0.430772012 | CAPSL; CAPSL; CAPSL; CAPSL | 37 | 5 |
| cg06894628 | 7.77E−10 | 3.30E−04 | −0.286004233 | 0.286004233 | loss | 0.600942421 | 0.314938188 | SLURP1 | 37 | 8 |
| cg06269928 | 7.13E−10 | 3.03E−04 | −0.28596894 | 0.28596894 | loss | 0.757463187 | 0.471494247 | | 37 | 1 |
| cg14803869 | 1.19E−09 | 5.07E−04 | −0.285922228 | 0.285922228 | loss | 0.864797487 | 0.578875259 | | 37 | 12 |
| cg08209422 | 8.47E−10 | 3.60E−04 | −0.285916073 | 0.285916073 | loss | 0.691804079 | 0.405888006 | RHBDL2; RHBDL2 | 37 | 1 |
| cg02458053 | 7.13E−10 | 3.03E−04 | −0.285905543 | 0.285905543 | loss | 0.912858408 | 0.626952865 | | 37 | 21 |
| cg04724659 | 7.13E−10 | 3.03E−04 | −0.285888213 | 0.285888213 | loss | 0.80795803 | 0.522069818 | CCSAP | 37 | 1 |
| cg10166889 | 1.54E−09 | 6.54E−04 | −0.285863844 | 0.285863844 | loss | 0.681166485 | 0.395302641 | | 37 | 5 |
| cg04105712 | 1.10E−09 | 4.65E−04 | −0.285797893 | 0.285797893 | loss | 0.812523317 | 0.526725424 | | 37 | 1 |
| cg18943588 | 2.69E−08 | 1.14E−02 | −0.285790873 | 0.285790873 | loss | 0.698468243 | 0.412677371 | | 37 | 10 |
| cg25943719 | 7.13E−10 | 3.03E−04 | −0.285766059 | 0.285766059 | loss | 0.664105906 | 0.378339847 | ALDH1A3 | 37 | 15 |
| cg27083329 | 1.30E−09 | 5.52E−04 | −0.285745385 | 0.285745385 | loss | 0.877217985 | 0.5914726 | | 37 | 15 |
| cg27629948 | 3.88E−09 | 1.65E−03 | −0.285704148 | 0.285704148 | loss | 0.930732136 | 0.645027988 | | 37 | 15 |
| cg19870370 | 7.13E−10 | 3.03E−04 | −0.285690385 | 0.285690385 | loss | 0.711838932 | 0.426148547 | CRYL1 | 37 | 13 |
| cg10570754 | 2.30E−08 | 9.75E−03 | −0.285660349 | 0.285660349 | loss | 0.858319996 | 0.572659647 | NTF3 | 37 | 12 |
| cg05030553 | 3.57E−09 | 1.52E−03 | −0.28564646 | 0.28564646 | loss | 0.704816777 | 0.419170318 | | 37 | 21 |
| cg11556592 | 8.47E−10 | 3.60E−04 | −0.285644327 | 0.285644327 | loss | 0.460723257 | 0.175078929 | LINC00482 | 37 | 17 |
| cg02477603 | 8.47E−10 | 3.60E−04 | −0.285644055 | 0.285644055 | loss | 0.397923208 | 0.112279153 | | 37 | 10 |
| cg23913963 | 7.13E−10 | 3.03E−04 | −0.285634757 | 0.285634757 | loss | 0.837828892 | 0.552194135 | RCC2 | 37 | 1 |
| cg24253618 | 7.84E−10 | 3.33E−04 | −0.285496616 | 0.285496616 | loss | 0.544281969 | 0.258785353 | | 37 | 1 |
| cg07897069 | 7.13E−10 | 3.03E−04 | −0.28548791 | 0.28548791 | loss | 0.761963204 | 0.476475294 | | 37 | 5 |
| cg19764594 | 7.13E−10 | 3.03E−04 | −0.285452755 | 0.285452755 | loss | 0.752179285 | 0.466726529 | LINC00606 | 37 | 3 |
| cg12439325 | 7.13E−10 | 3.03E−04 | −0.285449331 | 0.285449331 | loss | 0.44546366 | 0.160014329 | TMPRSS12 | 37 | 12 |
| cg24756378 | 3.28E−09 | 1.39E−03 | −0.285393711 | 0.285393711 | loss | 0.713811681 | 0.428417971 | | 37 | 14 |
| cg00627029 | 7.13E−10 | 3.03E−04 | −0.285312867 | 0.285312867 | loss | 0.362844923 | 0.077532055 | KIF19 | 37 | 17 |
| cg07636176 | 8.47E−10 | 3.60E−04 | −0.285211214 | 0.285211214 | loss | 0.667770449 | 0.382559235 | APOB | 37 | 2 |
| cg03472880 | 7.13E−10 | 3.03E−04 | −0.285210447 | 0.285210447 | loss | 0.790356842 | 0.505146394 | | 37 | 2 |
| cg13227481 | 7.13E−10 | 3.03E−04 | −0.285203248 | 0.285203248 | loss | 0.649987289 | 0.364784041 | DES | 37 | 2 |
| cg09239106 | 7.13E−10 | 3.03E−04 | −0.285172826 | 0.285172826 | loss | 0.699356226 | 0.4141834 | KCNH3 | 37 | 12 |
| cg02961934 | 7.13E−10 | 3.03E−04 | −0.285153003 | 0.285153003 | loss | 0.967368615 | 0.682215612 | | 37 | 7 |
| cg09517075 | 1.10E−09 | 4.65E−04 | −0.285099919 | 0.285099919 | loss | 0.813806166 | 0.528706247 | PIWIL2; PIWIL2 | 37 | 8 |
| cg10742957 | 7.13E−10 | 3.03E−04 | −0.285097503 | 0.285097503 | loss | 0.340079349 | 0.054981846 | FBXO17; FBXO17 | 37 | 19 |
| cg22933058 | 7.13E−10 | 3.03E−04 | −0.284928778 | 0.284928778 | loss | 0.568747613 | 0.283818835 | | 37 | 1 |
| cg16816874 | 7.77E−10 | 3.30E−04 | −0.284906969 | 0.284906969 | loss | 0.695344134 | 0.410437165 | GATA4 | 37 | 8 |
| cg18145090 | 1.99E−09 | 8.43E−04 | −0.284870852 | 0.284870852 | loss | 0.876017364 | 0.591146512 | GJB6; GJB6; GJB6; GJB6 | 37 | 13 |
| cg27166581 | 7.13E−10 | 3.03E−04 | −0.2848596 | 0.2848596 | loss | 0.4959997 | 0.2111401 | | 37 | 1 |
| cg13723983 | 7.13E−10 | 3.03E−04 | −0.284818907 | 0.284818907 | loss | 0.82238316 | 0.537564253 | NTMT1; NTMT1; C9orf50 | 37 | 9 |
| cg16331632 | 3.88E−09 | 1.65E−03 | −0.284722253 | 0.284722253 | loss | 0.716127906 | 0.431405653 | | 37 | 14 |
| cg16568641 | 3.28E−09 | 1.39E−03 | −0.284591253 | 0.284591253 | loss | 0.591317847 | 0.306726594 | LRRC6; LRRC6 | 37 | 8 |
| cg00806138 | 7.13E−10 | 3.03E−04 | −0.284527192 | 0.284527192 | loss | 0.795498715 | 0.510971524 | FOXE3 | 37 | 1 |
| cg06168149 | 7.13E−10 | 3.03E−04 | −0.284524009 | 0.284524009 | loss | 0.543894732 | 0.259370724 | GPD1; GPD1 | 37 | 12 |
| cg06928246 | 7.13E−10 | 3.03E−04 | −0.284482357 | 0.284482357 | loss | 0.702864198 | 0.418381841 | | 37 | 1 |
| cg07011961 | 6.89E−09 | 2.93E−03 | −0.284451748 | 0.284451748 | loss | 0.768929966 | 0.484478218 | PRDM9 | 37 | 5 |
| cg00693419 | 7.77E−10 | 3.30E−04 | −0.284437308 | 0.284437308 | loss | 0.681575996 | 0.397138688 | KCNMA1; KCNMA1; KCNMA1; | 37 | 10 |
| | | | | | | | | KCNMA1; KCNMA1; KCNMA1 | | |
| cg23639734 | 2.69E−08 | 1.14E−02 | −0.284394288 | 0.284394288 | loss | 0.783403906 | 0.499009618 | SOD3 | 37 | 4 |
| cg14414792 | 1.54E−09 | 6.54E−04 | −0.284375182 | 0.284375182 | loss | 0.843662747 | 0.559287565 | ATOH7; ATOH7 | 37 | 10 |
| cg02931685 | 7.13E−10 | 3.03E−04 | −0.284357526 | 0.284357526 | loss | 0.638378996 | 0.354021471 | | 37 | 19 |
| cg09889948 | 3.88E−09 | 1.65E−03 | −0.284353415 | 0.284353415 | loss | 0.548620468 | 0.264267053 | | 37 | 17 |
| cg02052758 | 2.16E−09 | 9.17E−04 | −0.284336706 | 0.284336706 | loss | 0.904753847 | 0.620417141 | | 37 | 7 |
| cg07238058 | 1.19E−09 | 5.07E−04 | −0.284304699 | 0.284304699 | loss | 0.769658575 | 0.485353876 | | 37 | 12 |
| cg26817546 | 7.84E−10 | 3.33E−04 | −0.28425924 | 0.28425924 | loss | 0.369505581 | 0.085246341 | | 37 | 5 |
| cg25154244 | 7.13E−10 | 3.03E−04 | −0.284250986 | 0.284250986 | loss | 0.531450815 | 0.247199829 | | 37 | 10 |
| cg10401489 | 7.13E−10 | 3.03E−04 | −0.284156804 | 0.284156804 | loss | 0.799517887 | 0.515361082 | TPGS1 | 37 | 19 |
| cg03604763 | 7.13E−10 | 3.03E−04 | −0.284156044 | 0.284156044 | loss | 0.665698968 | 0.381542924 | WDR63 | 37 | 1 |
| cg08932654 | 7.13E−10 | 3.03E−04 | −0.284151149 | 0.284151149 | loss | 0.509549113 | 0.225397965 | ORMDL3 | 37 | 17 |
| cg17515480 | 7.13E−10 | 3.03E−04 | −0.284081148 | 0.284081148 | loss | 0.395564364 | 0.111483216 | ZNF541 | 37 | 19 |
| cg03217115 | 1.01E−09 | 4.27E−04 | −0.284053626 | 0.284053626 | loss | 0.528484579 | 0.244430953 | | 37 | 2 |
| cg08891559 | 4.97E−09 | 2.11E−03 | −0.28404425 | 0.28404425 | loss | 0.558458421 | 0.274414171 | | 37 | 10 |
| cg25878149 | 7.13E−10 | 3.03E−04 | −0.28402303 | 0.28402303 | loss | 0.806551289 | 0.522528259 | | 37 | 2 |
| cg23737263 | 7.13E−10 | 3.03E−04 | −0.283949594 | 0.283949594 | loss | 0.824776194 | 0.5408266 | | 37 | 2 |
| cg19159759 | 7.13E−10 | 3.03E−04 | −0.283922287 | 0.283922287 | loss | 0.779574192 | 0.495651906 | CRYL1 | 37 | 13 |
| cg14010405 | 8.47E−10 | 3.60E−04 | −0.283918776 | 0.283918776 | loss | 0.835979353 | 0.552060576 | GTF2B | 37 | 1 |
| cg04248271 | 7.13E−10 | 3.03E−04 | −0.283887813 | 0.283887813 | loss | 0.35600183 | 0.072114017 | EBF1 | 37 | 5 |
| cg07075347 | 2.16E−09 | 9.17E−04 | −0.283837589 | 0.283837589 | loss | 0.429952232 | 0.146114644 | PGAM2 | 37 | 7 |
| cg13017614 | 7.13E−10 | 3.03E−04 | −0.283768436 | 0.283768436 | loss | 0.821006042 | 0.537237606 | FOXE3 | 37 | 1 |
| cg04850999 | 7.13E−10 | 3.03E−04 | −0.283768318 | 0.283768318 | loss | 0.711004083 | 0.427235765 | C10orf11 | 37 | 10 |
| cg08111158 | 7.13E−10 | 3.03E−04 | −0.283746342 | 0.283746342 | loss | 0.383520006 | 0.099773664 | | 37 | 1 |
| cg05661164 | 1.01E−09 | 4.27E−04 | −0.283726952 | 0.283726952 | loss | 0.809762881 | 0.526035929 | KRT83; KRT83 | 37 | 12 |
| cg26039829 | 7.13E−10 | 3.03E−04 | −0.283704151 | 0.283704151 | loss | 0.932309298 | 0.648605147 | PIWIL2; PIWIL2 | 37 | 8 |
| cg21467935 | 7.13E−10 | 3.03E−04 | −0.283698265 | 0.283698265 | loss | 0.837448836 | 0.553750571 | HSPA1A; HSPA1L | 37 | 6 |
| cg02931604 | 8.47E−10 | 3.60E−04 | −0.283672256 | 0.283672256 | loss | 0.628660509 | 0.344988253 | DPPA5 | 37 | 6 |
| cg24083746 | 7.13E−10 | 3.03E−04 | −0.283660178 | 0.283660178 | loss | 0.524663213 | 0.241003035 | CKM; CKM | 37 | 19 |
| cg10574365 | 7.84E−10 | 3.33E−04 | −0.283628874 | 0.283628874 | loss | 0.67598621 | 0.392357335 | | 37 | 1 |
| cg07865444 | 7.77E−10 | 3.30E−04 | −0.283621337 | 0.283621337 | loss | 0.838529296 | 0.554907959 | | 37 | 8 |
| cg24945462 | 8.11E−09 | 3.44E−03 | −0.283613881 | 0.283613881 | loss | 0.796341728 | 0.512727847 | LOC642852 | 37 | 21 |
| cg17000480 | 7.13E−10 | 3.03E−04 | −0.283603598 | 0.283603598 | loss | 0.68506584 | 0.401462241 | TTC40 | 37 | 10 |
| cg08888203 | 7.13E−10 | 3.03E−04 | −0.283507839 | 0.283507839 | loss | 0.975009668 | 0.691501829 | FANCD2OS; FANCD2OS; FANCD2OS; | 37 | 3 |
| | | | | | | | | FANCD2OS | | |
| cg06070578 | 1.19E−09 | 5.07E−04 | −0.283504545 | 0.283504545 | loss | 0.885328145 | 0.6018236 | | 37 | X |
| cg02042585 | 7.77E−10 | 3.30E−04 | −0.283499181 | 0.283499181 | loss | 0.748544675 | 0.465045494 | SLC22A3 | 37 | 6 |
| cg22374525 | 7.13E−10 | 3.03E−04 | −0.283493069 | 0.283493069 | loss | 0.521633328 | 0.238140259 | | 37 | 1 |
| cg07394701 | 1.99E−09 | 8.43E−04 | −0.283477519 | 0.283477519 | loss | 0.911439472 | 0.627961953 | NBPF10; LOC100288142 | 37 | 1 |
| cg11231735 | 1.99E−09 | 8.43E−04 | −0.283452994 | 0.283452994 | loss | 0.621733464 | 0.338280471 | | 37 | 2 |
| cg17129388 | 7.13E−10 | 3.03E−04 | −0.283397808 | 0.283397808 | loss | 0.777443402 | 0.494045594 | NGFR | 37 | 17 |
| cg00001099 | 2.26E−08 | 9.58E−03 | −0.283378362 | 0.283378362 | loss | 0.75333085 | 0.469952488 | PSKH2 | 37 | 8 |
| cg14062977 | 7.13E−10 | 3.03E−04 | −0.283365541 | 0.283365541 | loss | 0.788817617 | 0.505452076 | C90rf171; C9orf171 | 37 | 9 |
| cg05157195 | 7.13E−10 | 3.03E−04 | −0.283304942 | 0.283304942 | loss | 0.639862389 | 0.356557447 | | 37 | 6 |
| cg24932660 | 7.13E−10 | 3.03E−04 | −0.283244123 | 0.283244123 | loss | 0.472459528 | 0.189215405 | CD164L2 | 37 | 1 |
| cg01570972 | 7.13E−10 | 3.03E−04 | −0.283240444 | 0.283240444 | loss | 0.479653679 | 0.196413235 | GRHL1 | 37 | 2 |
| cg15677087 | 7.13E−10 | 3.03E−04 | −0.283231742 | 0.283231742 | loss | 0.54139193 | 0.258160188 | SLC17A9 | 37 | 20 |
| cg02707176 | 3.40E−08 | 1.44E−02 | 0.28321165 | 0.28321165 | gain | 0.429720679 | 0.712932329 | PCDHGB4; PCDHGA1; PCDHGA10; | 37 | 5 |
| | | | | | | | | PCDHGA2; PCDHGA3; | | |
| | | | | | | | | PCDHGA4; PCDHGA5; | | |
| | | | | | | | | PCDHGA6; PCDHGA7; PCDHGA9; | | |
| | | | | | | | | PCDHGB1; PCDKGB2; | | |
| | | | | | | | | PCDHGB3; PCDHGB5; PCDHGB6; | | |
| | | | | | | | | PCDHGB7; PCDHGA8; | | |
| | | | | | | | | PCDHGB7 | | |
| cg02197228 | 8.65E−10 | 3.67E−04 | −0.283160125 | 0.283160125 | loss | 0.754715831 | 0.471555706 | ASIC4 | 37 | 2 |
| cg14307477 | 7.77E−10 | 3.30E−04 | −0.283151773 | 0.283151773 | loss | 0.821084649 | 0.537932876 | KLK15; KLK15; KLK15; KLK15 | 37 | 19 |
| cg08310581 | 9.23E−10 | 3.92E−04 | −0.283123149 | 0.283123149 | loss | 0.586403213 | 0.303280065 | ARHGEF4; ARHGEF4 | 37 | 2 |
| cg03295928 | 7.13E−10 | 3.03E−04 | −0.283072569 | 0.283072569 | loss | 0.342794832 | 0.059722263 | RTKN | 37 | 2 |
| cg09652312 | 3.57E−09 | 1.52E−03 | −0.283042788 | 0.283042788 | loss | 0.554749481 | 0.271706694 | | 37 | 7 |
| cg22745354 | 7.13E−10 | 3.03E−04 | −0.282998008 | 0.282998008 | loss | 0.640656308 | 0.3576583 | | 37 | 2 |
| cg08246800 | 7.13E−10 | 3.03E−04 | −0.282985153 | 0.282985153 | loss | 0.742833336 | 0.459848182 | PLEKHB1; PLEKHB1 | 37 | 11 |
| cg07045089 | 7.13E−10 | 3.03E−04 | −0.282981354 | 0.282981354 | loss | 0.84983486 | 0.566853506 | TP73; TP73; TP73; TP73; TP73; | 37 | 1 |
| | | | | | | | | TP73; TP73; TP73; TP73; | | |
| | | | | | | | | TP73; TP73; TP73; TP73 | | |
| cg27350104 | 7.13E−10 | 3.03E−04 | −0.282968651 | 0.282968651 | loss | 0.69568304 | 0.412714388 | | 37 | 11 |
| cg19392379 | 1.54E−09 | 6.54E−04 | −0.282958723 | 0.282958723 | loss | 0.818765575 | 0.535806853 | TACC2; TACC2 | 37 | 10 |
| cg17519938 | 2.56E−09 | 1.09E−03 | −0.28294301 | 0.28294301 | loss | 0.621190451 | 0.338247441 | LRRC47 | 37 | 1 |
| cg09853936 | 7.13E−10 | 3.03E−04 | −0.282914189 | 0.282914189 | loss | 0.690450819 | 0.407536629 | ARHGEF16 | 37 | 1 |
| cg10872641 | 7.13E−10 | 3.03E−04 | −0.282906511 | 0.282906511 | loss | 0.689661923 | 0.406755412 | MTERF | 37 | 7 |
| cg15117754 | 7.13E−10 | 3.03E−04 | −0.282902084 | 0.282902084 | loss | 0.854755566 | 0.571853482 | FANCD2OS; FANCD2OS; FANCD2OS; | 37 | 3 |
| | | | | | | | | FANCD2OS | | |
| cg19297232 | 1.99E−09 | 8.43E−04 | −0.28286253 | 0.28286253 | loss | 0.693293725 | 0.410431194 | SMPD3 | 37 | 16 |
| cg25712005 | 7.13E−10 | 3.03E−04 | −0.282857442 | 0.282857442 | loss | 0.536012936 | 0.253155494 | ELN; ELN; ELN; ELN; ELN; ELN; | 37 | 7 |
| | | | | | | | | ELN; ELN; ELN; ELN; ELN; | | |
| | | | | | | | | ELN; ELN | | |
| cg25301173 | 3.57E−09 | 1.52E−03 | −0.282764189 | 0.282764189 | loss | 0.746654936 | 0.463890747 | | 37 | 2 |
| cg07745344 | 9.23E−10 | 3.92E−04 | −0.282760322 | 0.282760322 | loss | 0.823133898 | 0.540373576 | | 37 | 8 |
| cg07651048 | 7.13E−10 | 3.03E−04 | −0.282690489 | 0.282690489 | loss | 0.802631989 | 0.5199415 | | 37 | 12 |
| cg12101586 | 1.01E−09 | 4.27E−04 | −0.282678535 | 0.282678535 | loss | 0.480843406 | 0.198164871 | CYP1A1 | 37 | 15 |
| cg14299483 | 8.47E−10 | 3.60E−04 | −0.282676216 | 0.282676216 | loss | 0.751522498 | 0.468846282 | | 37 | 14 |
| cg05985317 | 7.13E−10 | 3.03E−04 | −0.282669762 | 0.282669762 | loss | 0.454733257 | 0.172063494 | PRRT1 | 37 | 6 |
| cg19605931 | 3.02E−09 | 1.28E−03 | −0.282656118 | 0.282656118 | loss | 0.814774347 | 0.532118229 | | 37 | 18 |
| cg01803844 | 7.13E−10 | 3.03E−04 | −0.282628841 | 0.282628841 | loss | 0.845244764 | 0.552615924 | | 37 | 17 |
| cg26820168 | 3.02E−09 | 1.28E−03 | −0.282602967 | 0.282602967 | loss | 0.562349032 | 0.279746065 | FAM83A; FAM83A | 37 | 8 |
| cg26586027 | 4.67E−09 | 1.98E−03 | −0.282540051 | 0.282540051 | loss | 0.835117588 | 0.552577538 | | 37 | 18 |
| cg22700246 | 7.48E−09 | 3.18E−03 | −0.282512895 | 0.282512895 | loss | 0.567572972 | 0.285060076 | | 37 | 15 |
| cg09590377 | 7.77E−10 | 3.30E−04 | −0.282465147 | 0.282465147 | loss | 0.332569708 | 0.050104561 | | 37 | 2 |
| cg04452896 | 9.23E−10 | 3.92E−04 | −0.282423654 | 0.282423654 | loss | 0.933872283 | 0.651448629 | | 37 | 11 |
| cg17391620 | 9.94E−08 | 4.22E−02 | −0.282409913 | 0.282409913 | loss | 0.776731808 | 0.494321894 | VWA7 | 37 | 6 |
| cg18052665 | 7.84E−10 | 3.33E−04 | −0.2823504 | 0.2823504 | loss | 0.9310677 | 0.6487173 | DPPA5; DPPA5 | 37 | 6 |
| cg15139452 | 7.13E−10 | 3.03E−04 | −0.282235694 | 0.282235694 | loss | 0.835530111 | 0.553294418 | | 37 | 1 |
| cg18318818 | 7.13E−10 | 3.03E−04 | −0.28223002 | 0.28223002 | loss | 0.473153534 | 0.190923514 | CACNA1G; CACNA1G; CACNA1G; | 37 | 17 |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G- | | |
| | | | | | | | | AS1; CACNA1G; CACNA1G; | | |
| | | | | | | | | CACNA1G; CACNA1G; CACNA1G | | |
| cg22199969 | 9.23E−10 | 3.92E−04 | −0.28220277 | 0.28220277 | loss | 0.423318666 | 0.141115896 | ALPI | 37 | 2 |
| cg10403849 | 7.13E−10 | 3.03E−04 | −0.282184458 | 0.282184458 | loss | 0.502844775 | 0.220660318 | TET1 | 37 | 10 |
| cg03483944 | 7.13E−10 | 3.03E−04 | −0.282158689 | 0.282158689 | loss | 0.747032972 | 0.464874282 | FAM71E2; FAM71E2 | 37 | 19 |
| cg24363820 | 4.97E−09 | 2.11E−03 | −0.28214541 | 0.28214541 | loss | 0.517493745 | 0.235348335 | CPT1B; CPT1B; CPT1B; CPT1B; | 37 | 22 |
| | | | | | | | | CPT1B; CPT1B; CPT1B; | | |
| | | | | | | | | CPT1B; CPT1B; CPT1B; CHKB- | | |
| | | | | | | | | CPT1B | | |
| cg00448720 | 2.16E−09 | 9.17E−04 | −0.282104431 | 0.282104431 | loss | 0.769101413 | 0.486996982 | C1QTNF9 | 37 | 13 |
| cg25137841 | 7.13E−10 | 3.03E−04 | −0.28204585 | 0.28204585 | loss | 0.647628738 | 0.365582888 | PLA2G3; PLA2G3 | 37 | 22 |
| cg26172342 | 7.13E−10 | 3.03E−04 | −0.281964595 | 0.281964595 | loss | 0.760630372 | 0.478665776 | ANK1; ANK1; ANK1; ANK1; | 37 | 8 |
| | | | | | | | | ANK1 | | |
| cg26343510 | 7.13E−10 | 3.03E−04 | −0.281961419 | 0.281961419 | loss | 0.423333806 | 0.141372387 | P2RY6; P2RY6; P2RY6; P2RY6; | 37 | 11 |
| | | | | | | | | P2RY6 | | |
| cg21593409 | 7.13E−10 | 3.03E−04 | −0.281953236 | 0.281953236 | loss | 0.552544494 | 0.270591259 | IL17C | 37 | 16 |
| cg06222062 | 7.13E−10 | 3.03E−04 | −0.281918506 | 0.281918506 | loss | 0.543449358 | 0.261530853 | PLA2G5 | 37 | 1 |
| cg19309616 | 1.83E−09 | 7.75E−04 | −0.281906263 | 0.281906263 | loss | 0.762031492 | 0.480125229 | | 37 | 1 |
| cg26940100 | 7.13E−10 | 3.03E−04 | −0.281831632 | 0.281831632 | loss | 0.824189538 | 0.542357906 | OBSCN; OBSCN; OBSCN; C1orf145; | 37 | 1 |
| | | | | | | | | C1orf145 | | |
| cg11857646 | 7.13E−10 | 3.03E−04 | −0.281796225 | 0.281796225 | loss | 0.526910802 | 0.245114576 | KCNH2; KCNH2 | 37 | 7 |
| cg19602315 | 7.13E−10 | 3.03E−04 | −0.281784882 | 0.281784882 | loss | 0.6333849 | 0.351600018 | C17orf75 | 37 | 17 |
| cg16970604 | 9.23E−10 | 3.92E−04 | −0.281716903 | 0.281716903 | loss | 0.862940726 | 0.581223824 | LOC100505532 | 37 | 8 |
| cg18709881 | 1.19E−09 | 5.07E−04 | −0.2816931 | 0.2816931 | loss | 0.838233711 | 0.556540612 | | 37 | 18 |
| cg08659204 | 7.13E−10 | 3.03E−04 | −0.281676234 | 0.281676234 | loss | 0.621158187 | 0.339481953 | | 37 | 17 |
| cg06200514 | 1.83E−09 | 7.75E−04 | −0.281666683 | 0.281666683 | loss | 0.919232942 | 0.637566259 | NLRP14 | 37 | 11 |
| cg08707536 | 9.23E−10 | 3.92E−04 | −0.281637442 | 0.281637442 | loss | 0.76460663 | 0.482969188 | | 37 | 6 |
| cg01202519 | 8.47E−10 | 3.60E−04 | −0.281594258 | 0.281594258 | loss | 0.521214464 | 0.239620206 | | 37 | 11 |
| cg27016974 | 1.02E−09 | 4.33E−04 | −0.281539343 | 0.281539343 | loss | 0.924259902 | 0.642720559 | PRSS42; PRSS42 | 37 | 3 |
| cg03996861 | 1.01E−09 | 4.27E−04 | −0.281525513 | 0.281525513 | loss | 0.503797172 | 0.222271659 | | 37 | 8 |
| cg21484547 | 7.13E−10 | 3.03E−04 | −0.281474282 | 0.281474282 | loss | 0.681053664 | 0.399579382 | | 37 | 13 |
| cg02744588 | 7.13E−10 | 3.03E−04 | −0.281447832 | 0.281447832 | loss | 0.689596343 | 0.408148512 | CCDC114 | 37 | 19 |
| cg14213394 | 4.29E−08 | 1.82E−02 | −0.281446682 | 0.281446682 | loss | 0.452064475 | 0.170617794 | | 37 | 8 |
| cg20317418 | 1.10E−09 | 4.65E−04 | −0.281412464 | 0.281412464 | loss | 0.894599258 | 0.613186794 | | 37 | 15 |
| cg17344906 | 8.47E−10 | 3.60E−04 | −0.281390708 | 0.281390708 | loss | 0.621379564 | 0.339988856 | NFIX; NFIX; NFIX | 37 | 19 |
| cg21144493 | 7.13E−10 | 3.03E−04 | −0.281382548 | 0.281382548 | loss | 0.4247112 | 0.143328652 | CAMTA1 | 37 | 1 |
| cg08767820 | 1.42E−09 | 6.01E−04 | −0.281347735 | 0.281347735 | loss | 0.402623585 | 0.12127585 | | 37 | 7 |
| cg12626589 | 7.13E−10 | 3.03E−04 | −0.281307271 | 0.281307271 | loss | 0.897073789 | 0.615766518 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg16877087 | 7.13E−10 | 3.03E−04 | −0.281239416 | 0.281239416 | loss | 0.91250014 | 0.531260724 | | 37 | 2 |
| cg04518195 | 7.13E−10 | 3.03E−04 | −0.281236239 | 0.281236239 | loss | 0.681318515 | 0.400082276 | LRRC4B | 37 | 19 |
| cg04833011 | 1.81E−08 | 7.69E−03 | −0.281181837 | 0.281181837 | loss | 0.807919155 | 0.526737318 | | 37 | 15 |
| cg25686793 | 7.13E−10 | 3.03E−04 | −0.281178585 | 0.281178585 | loss | 0.843698485 | 0.5625199 | CRX | 37 | 19 |
| cg13570656 | 1.30E−09 | 5.52E−04 | −0.281154544 | 0.281154544 | loss | 0.429151515 | 0.147996971 | CYP1A1 | 37 | 15 |
| cg04613258 | 7.13E−10 | 3.03E−04 | −0.281140325 | 0.281140325 | loss | 0.526884902 | 0.245744576 | NGFR; LOC100288866 | 37 | 17 |
| cg20401945 | 7.13E−10 | 3.03E−04 | −0.281084587 | 0.281084587 | loss | 0.535786487 | 0.2547019 | ASPHD1 | 37 | 16 |
| cg11883836 | 7.13E−10 | 3.03E−04 | −0.281068464 | 0.281068464 | loss | 0.626352458 | 0.345293994 | LINC00880 | 37 | 3 |
| cg25534953 | 7.13E−10 | 3.03E−04 | −0.281064307 | 0.281064307 | loss | 0.471913294 | 0.190848988 | NEURL3 | 37 | 2 |
| cg08374687 | 7.13E−10 | 3.03E−04 | −0.28105253 | 0.28105253 | loss | 0.951319142 | 0.670266612 | ADAD1; ADAD1; ADAD1 | 37 | 4 |
| cg15278109 | 7.13E−10 | 3.03E−04 | −0.281024492 | 0.281024492 | loss | 0.365352057 | 0.084327565 | | 37 | 10 |
| cg19535358 | 2.12E−08 | 9.01E−03 | −0.281022408 | 0.281022408 | loss | 0.503999585 | 0.522977176 | | 37 | 13 |
| cg21749424 | 7.13E−10 | 3.03E−04 | −0.280983999 | 0.280983999 | loss | 0.597269181 | 0.316285182 | | 37 | 6 |
| cg21867110 | 7.13E−10 | 3.03E−04 | −0.280981422 | 0.280981422 | loss | 0.573581987 | 0.292600565 | | 37 | 19 |
| cg05828191 | 1.73E−09 | 7.34E−04 | −0.280971791 | 0.280971791 | loss | 0.776787466 | 0.495815675 | WDR59 | 37 | 16 |
| cg11146935 | 7.13E−10 | 3.03E−04 | −0.280921816 | 0.280921816 | loss | 0.895053434 | 0.614131618 | | 37 | 15 |
| cg27547703 | 4.61E−08 | 1.96E−02 | −0.280891759 | 0.280891759 | loss | 0.953366077 | 0.672474318 | TDRD1 | 37 | 10 |
| cg01430344 | 9.23E−10 | 3.92E−04 | −0.280859224 | 0.280859224 | loss | 0.526510036 | 0.245550812 | | 37 | 2 |
| cg04205744 | 7.77E−10 | 3.30E−04 | −0.280838643 | 0.280838643 | loss | 0.829034502 | 0.548195859 | | 37 | 16 |
| cg25612391 | 7.13E−10 | 3.03E−04 | −0.280817609 | 0.280817609 | loss | 0.885676568 | 0.604858959 | SLC25A42 | 37 | 19 |
| cg18434354 | 7.13E−10 | 3.03E−04 | −0.280797422 | 0.280797422 | loss | 0.76074744 | 0.479950018 | LINC01101; LINC01101 | 37 | 2 |
| cg03333149 | 3.11E−13 | 1.32E−07 | −0.280730533 | 0.280730533 | loss | 0.938252206 | 0.657521673 | | 37 | 12 |
| cg19197440 | 7.13E−10 | 3.03E−04 | −0.280705604 | 0.280705604 | loss | 0.53607444 | 0.255368835 | | 37 | 17 |
| cg10687107 | 7.13E−10 | 3.03E−04 | −0.280677068 | 0.280677068 | loss | 0.590724068 | 0.310047 | | 37 | 8 |
| cg01401534 | 7.13E−10 | 3.03E−04 | −0.280622551 | 0.280622551 | loss | 0.459305151 | 0.1786826 | C6orf201; FAM217A; FAM217A; | 37 | 6 |
| | | | | | | | | C6orf201; C6orf201 | | |
| cg06637774 | 7.13E−10 | 3.03E−04 | −0.280602249 | 0.280602249 | loss | 0.38839973 | 0.107797481 | P2RY6; P2RY6; P2RY6; P2RY6; | 37 | 11 |
| | | | | | | | | P2RY6 | | |
| cg15813594 | 3.88E−09 | 1.65E−03 | −0.280580755 | 0.280580755 | loss | 0.823673943 | 0.543093188 | EGFLAM; EGFLAM; EGFLAM; | 37 | 5 |
| | | | | | | | | EGFLAM | | |
| cg02452639 | 1.30E−09 | 5.52E−04 | −0.280540927 | 0.280540927 | loss | 0.814545162 | 0.534004235 | | 37 | 1 |
| cg25120325 | 5.41E−08 | 2.30E−02 | −0.280526743 | 0.280526743 | loss | 0.825300249 | 0.544773506 | PNLIPRP2; PNLIPRP2 | 37 | 10 |
| cg27108424 | 7.13E−10 | 3.03E−04 | −0.280500272 | 0.280500272 | loss | 0.908618113 | 0.628117841 | | 37 | 17 |
| cg27649194 | 7.13E−10 | 3.03E−04 | −0.280485993 | 0.280485993 | loss | 0.417762194 | 0.137276202 | CACNA2D4 | 37 | 12 |
| cg24489344 | 7.13E−10 | 3.03E−04 | −0.280443055 | 0.280443055 | loss | 0.982003243 | 0.701560188 | | 37 | 22 |
| cg05365033 | 5.41E−08 | 2.30E−02 | −0.280405347 | 0.280405347 | loss | 0.711077964 | 0.430672618 | CRTC1; CRTC1 | 37 | 19 |
| cg08221781 | 7.13E−10 | 3.03E−04 | −0.280324884 | 0.280324884 | loss | 0.651259549 | 0.370934665 | RHOD | 37 | 11 |
| cg04865929 | 1.89E−09 | 8.02E−04 | −0.280267661 | 0.280267661 | loss | 0.715975274 | 0.435707613 | GJC1; GJC1 | 37 | 17 |
| cg14080129 | 3.88E−09 | 1.65E−03 | −0.280095998 | 0.280095998 | loss | 0.91136154 | 0.631265541 | | 37 | 9 |
| cg26706187 | 6.35E−09 | 2.70E−03 | −0.280074017 | 0.280074017 | loss | 0.936513664 | 0.656439647 | | 37 | 11 |
| cg21067023 | 2.78E−09 | 1.18E−03 | −0.28006047 | 0.28006047 | loss | 0.680458106 | 0.400397635 | COLEC12 | 37 | 18 |
| cg03539001 | 7.13E−10 | 3.03E−04 | −0.280047851 | 0.280047851 | loss | 0.509508745 | 0.229460894 | MORF4L1; MORF4L1; MORF4L1; | 37 | 15 |
| | | | | | | | | MORF4L1; MORF4L1 | | |
| cg19461383 | 1.01E−09 | 4.27E−04 | −0.280030264 | 0.280030264 | loss | 0.853427958 | 0.573397694 | NOX5; NOX5; NOX5; MIR548H4; | 37 | 15 |
| | | | | | | | | NOX5; NOX5 | | |
| cg15620905 | 7.13E−10 | 3.03E−04 | −0.280018264 | 0.280018264 | loss | 0.611080411 | 0.331062147 | PTPRF; PTPRF | 37 | 1 |
| cg12124927 | 7.13E−10 | 3.03E−04 | −0.280014451 | 0.280014451 | loss | 0.780347251 | 0.5003328 | GRIK4; GRIK4; GRIK4 | 37 | 11 |
| cg06521653 | 1.81E−08 | 7.69E−03 | −0.280014085 | 0.280014085 | loss | 0.471083636 | 0.191069551 | | 37 | 16 |
| cg19884634 | 7.13E−10 | 3.03E−04 | −0.279978316 | 0.279978316 | loss | 0.775466934 | 0.495488618 | PRRT4; PRRT4 | 37 | 7 |
| cg14395298 | 1.47E−09 | 6.24E−04 | −0.279942925 | 0.279942925 | loss | 0.381247833 | 0.101304909 | | 37 | 13 |
| cg19364351 | 1.19E−09 | 5.07E−04 | −0.279922724 | 0.279922724 | loss | 0.76494213 | 0.485019406 | CCKBR | 37 | 11 |
| cg10052908 | 8.47E−10 | 3.60E−04 | −0.279906964 | 0.279906964 | loss | 0.933519958 | 0.653612994 | DPPA5 | 37 | 6 |
| cg00206639 | 1.99E−09 | 8.43E−04 | −0.279865621 | 0.279865621 | loss | 0.441535387 | 0.161669765 | | 37 | 15 |
| cg19727499 | 1.73E−09 | 7.34E−04 | −0.279758059 | 0.279758059 | loss | 0.861297453 | 0.581539394 | PPFIA3; PPFIA3 | 37 | 19 |
| cg20512303 | 7.13E−10 | 3.03E−04 | −0.279732521 | 0.279732521 | loss | 0.778159709 | 0.498427188 | PDLIM4; PDLIM4 | 37 | 5 |
| cg00660384 | 7.77E−10 | 3.30E−04 | −0.279720739 | 0.279720739 | loss | 0.811757404 | 0.532036665 | RASGEF1A | 37 | 10 |
| cg22276371 | 7.13E−10 | 3.03E−04 | −0.279712893 | 0.279712893 | loss | 0.434590781 | 0.154877888 | CHRND; CHRND; CHRND; CHRND | 37 | 2 |
| cg11976911 | 3.28E−09 | 1.39E−03 | −0.279705073 | 0.279705073 | loss | 0.500052226 | 0.220347153 | | 37 | 11 |
| cg10934807 | 2.16E−09 | 9.17E−04 | −0.279704289 | 0.279704289 | loss | 0.757828372 | 0.478124082 | RGPD8; RGPD5 | 37 | 2 |
| cg26333618 | 7.13E−10 | 3.03E−04 | −0.279698051 | 0.279698051 | loss | 0.683152798 | 0.403454747 | | 37 | 12 |
| cg17263206 | 7.13E−10 | 3.03E−04 | −0.279656113 | 0.279656113 | loss | 0.482554025 | 0.202897912 | | 37 | 5 |
| cg10161743 | 7.13E−10 | 3.03E−04 | −0.279633145 | 0.279633145 | loss | 0.489057262 | 0.209424118 | GRIN2D | 37 | 19 |
| cg13809236 | 7.13E−10 | 3.03E−04 | −0.279627588 | 0.279527588 | loss | 0.710385217 | 0.430857629 | MAPK8IP1 | 37 | 11 |
| cg00506049 | 7.13E−10 | 3.03E−04 | −0.279514321 | 0.279514321 | loss | 0.608576226 | 0.329061906 | SRRM3 | 37 | 7 |
| cg08701183 | 1.01E−09 | 4.27E−04 | −0.279502036 | 0.279502036 | loss | 0.514781972 | 0.235279935 | | 37 | 7 |
| cg03987199 | 7.13E−10 | 3.03E−04 | −0.279471918 | 0.279471918 | loss | 0.869454242 | 0.589982324 | OPRD1 | 37 | 1 |
| cg10892403 | 7.13E−10 | 3.03E−04 | −0.279458033 | 0.279458033 | loss | 0.408978809 | 0.129520776 | CHRM1 | 37 | 11 |
| cg26643476 | 9.54E−09 | 4.05E−03 | −0.279435035 | 0.279435035 | loss | 0.840605511 | 0.561170476 | HCAR1 | 37 | 12 |
| cg03236170 | 1.01E−09 | 4.27E−04 | −0.279432822 | 0.279432822 | loss | 0.486426551 | 0.206993729 | | 37 | 16 |
| cg24639679 | 3.12E−09 | 1.32E−03 | −0.279417947 | 0.279417947 | loss | 0.705715117 | 0.426297171 | | 37 | 6 |
| cg02716646 | 7.77E−10 | 3.30E−04 | −0.279399683 | 0.279399683 | loss | 0.510878572 | 0.231478888 | POMC; POMC | 37 | 2 |
| cg00732378 | 7.13E−10 | 3.03E−04 | −0.279391652 | 0.279391652 | loss | 0.567619434 | 0.288227782 | | 37 | 16 |
| cg08772003 | 4.21E−09 | 1.79E−03 | −0.27937141 | 0.27937141 | loss | 0.492583798 | 0.213212388 | AS3MT; C10orf32-ASMT | 37 | 10 |
| cg08966208 | 7.13E−10 | 3.03E−04 | −0.279360337 | 0.279360337 | loss | 0.530490479 | 0.251130142 | IFITM10; MOB2 | 37 | 11 |
| cg25101936 | 7.13E−10 | 3.03E−04 | −0.279355453 | 0.279355453 | loss | 0.633321506 | 0.353966053 | ZBTB16 | 37 | 11 |
| cg13010014 | 7.13E−10 | 3.03E−04 | −0.279350686 | 0.279350686 | loss | 0.593149404 | 0.313798718 | ROBO3 | 37 | 11 |
| cg11884274 | 7.13E−10 | 3.03E−04 | −0.279343807 | 0.279343807 | loss | 0.762491525 | 0.483147718 | NOTCH4 | 37 | 5 |
| cg13228642 | 7.13E−10 | 3.03E−04 | −0.279321402 | 0.279321402 | loss | 0.543447002 | 0.2641256 | IER5 | 37 | 1 |
| cg16836311 | 7.77E−10 | 3.30E−04 | −0.279298046 | 0.279298046 | loss | 0.630519275 | 0.351221229 | MAN1C1 | 37 | 1 |
| cg22795769 | 7.13E−10 | 3.03E−04 | −0.279241647 | 0.279241647 | loss | 0.666759658 | 0.387518012 | MYO1C; MYO1C; MYO1C | 37 | 17 |
| cg08656770 | 7.13E−10 | 3.03E−04 | −0.279236867 | 0.279236867 | loss | 0.590823096 | 0.311586229 | | 37 | 6 |
| cg08274552 | 7.13E−10 | 3.03E−04 | −0.279191816 | 0.279191816 | loss | 0.776639451 | 0.497447635 | SLC4A3; SLC4A3; SLC4A3 | 37 | 2 |
| cg24892948 | 7.13E−10 | 3.03E−04 | −0.279162778 | 0.279162778 | loss | 0.727998755 | 0.448835976 | DMKN; DMKN; DMKN; DMKN; | 37 | 19 |
| | | | | | | | | DMKN; DMKN; DMKN; | | |
| | | | | | | | | DMKN; DMKN | | |
| cg07803814 | 5.41E−08 | 2.30E−02 | −0.279159858 | 0.279159858 | loss | 0.896438881 | 0.617279024 | VGLL4; VGLL4 | 37 | 3 |
| cg19263607 | 7.13E−10 | 3.03E−04 | −0.279156259 | 0.279156259 | loss | 0.5009584 | 0.221802141 | | 37 | 7 |
| cg04598670 | 7.34E−08 | 3.12E−02 | −0.279130326 | 0.279130326 | loss | 0.585712596 | 0.307582271 | | 37 | 7 |
| cg16129526 | 7.77E−10 | 3.30E−04 | −0.279071766 | 0.279071766 | loss | 0.754884319 | 0.475812553 | PLEKHD1 | 37 | 14 |
| cg12686920 | 3.28E−09 | 1.39E−03 | −0.279063734 | 0.279063734 | loss | 0.654813945 | 0.375750212 | MEOX1; MEOX1; MEOX1 | 37 | 17 |
| cg26841862 | 1.54E−09 | 6.54E−04 | −0.27906046 | 0.27906046 | loss | 0.550511577 | 0.271451118 | | 37 | 12 |
| cg08538032 | 7.77E−10 | 3.30E−04 | −0.279019968 | 0.279019968 | loss | 0.489208815 | 0.210188847 | ELAVL4; ELAVL4; ELAVL4; ELAVL4; | 37 | 1 |
| | | | | | | | | ELAVL4 | | |
| cg00324725 | 4.57E−09 | 1.94E−03 | −0.278968012 | 0.278968012 | loss | 0.570089042 | 0.291121029 | | 37 | 16 |
| cg15468668 | 7.13E−10 | 3.03E−04 | −0.278954918 | 0.278954918 | loss | 0.838501783 | 0.559546865 | ASGR1; ASGR1; ASGR1; ASGR1 | 37 | 17 |
| cg11024722 | 2.16E−09 | 9.17E−04 | −0.278917713 | 0.278917713 | loss | 0.68905503 | 0.410137318 | UCN3 | 37 | 10 |
| cg22382021 | 1.30E−09 | 5.52E−04 | −0.278890246 | 0.278890246 | loss | 0.723742417 | 0.444852171 | C8orf82; ARHGAP39 | 37 | 8 |
| cg26396721 | 1.10E−09 | 4.65E−04 | −0.278869466 | 0.278869466 | loss | 0.94645566 | 0.667586194 | | 37 | 2 |
| cg25883020 | 7.13E−10 | 3.03E−04 | −0.278853204 | 0.278853204 | loss | 0.730107909 | 0.451254706 | NKX6-3; NKX6-3 | 37 | 8 |
| cg11262850 | 7.13E−10 | 3.03E−04 | −0.278841086 | 0.278841086 | loss | 0.537768145 | 0.258927059 | | 37 | 5 |
| cg24580146 | 2.35E−09 | 9.98E−04 | −0.278807837 | 0.278807837 | loss | 0.790505349 | 0.511697512 | DNAH2 | 37 | 17 |
| cg03162994 | 7.77E−10 | 3.30E−04 | −0.278803924 | 0.278803924 | loss | 0.691578989 | 0.412775065 | PSMA8; PSMA8; PSMA8 | 37 | 18 |
| cg00316312 | 7.13E−10 | 3.03E−04 | −0.278779059 | 0.278779059 | loss | 0.817609806 | 0.538830747 | GPR123 | 37 | 10 |
| cg24184022 | 2.56E−09 | 1.09E−03 | −0.278754977 | 0.278754977 | loss | 0.886737542 | 0.607982565 | | 37 | 7 |
| cg05767720 | 7.13E−10 | 3.03E−04 | −0.278731842 | 0.278731842 | loss | 0.396149909 | 0.117418067 | PRRT1 | 37 | 6 |
| cg19403023 | 2.35E−09 | 9.98E−04 | −0.27870296 | 0.27870296 | loss | 0.739682766 | 0.460979806 | PRSS41 | 37 | 16 |
| cg20699586 | 1.01E−09 | 4.27E−04 | −0.278684526 | 0.278684526 | loss | 0.342086809 | 0.063402283 | | 37 | 1 |
| cg08358943 | 7.13E−10 | 3.03E−04 | −0.278648999 | 0.278648999 | loss | 0.660372228 | 0.381723229 | WNT3 | 37 | 17 |
| cg26917745 | 7.13E−10 | 3.03E−04 | −0.278607488 | 0.278607488 | loss | 0.594960364 | 0.316352876 | | 37 | 7 |
| cg00063006 | 7.77E−10 | 3.30E−04 | −0.278562999 | 0.278562999 | loss | 0.670576664 | 0.392013665 | | 37 | 6 |
| cg11948766 | 1.54E−08 | 6.56E−03 | −0.278484585 | 0.278484585 | loss | 0.973990243 | 0.695505659 | | 37 | 8 |
| cg16167422 | 7.77E−10 | 3.30E−04 | −0.278479853 | 0.278479853 | loss | 0.731874436 | 0.453394582 | | 37 | 7 |
| cg05034195 | 7.13E−10 | 3.03E−04 | −0.278463855 | 0.278463855 | loss | 0.315495479 | 0.037031625 | | 37 | 2 |
| cg14042396 | 7.13E−10 | 3.03E−04 | −0.278439843 | 0.278439843 | loss | 0.430195008 | 0.151755165 | | 37 | 2 |
| cg22517356 | 7.13E−10 | 3.03E−04 | −0.278437387 | 0.278437387 | loss | 0.605255549 | 0.226818162 | TMEM88B | 37 | 1 |
| cg16517358 | 1.01E−09 | 4.27E−04 | −0.278417956 | 0.278417956 | loss | 0.855808279 | 0.577390324 | | 37 | 11 |
| cg17200137 | 1.68E−09 | 7.12E−04 | −0.278244228 | 0.278244228 | loss | 0.830692734 | 0.552448506 | GDF2; GDF2 | 37 | 10 |
| cg13468625 | 7.13E−10 | 3.03E−04 | −0.278173468 | 0.278173468 | loss | 0.688487362 | 0.410313894 | EXD3 | 37 | 9 |
| cg01807241 | 7.13E−10 | 3.03E−04 | −0.278161618 | 0.278161618 | loss | 0.367791826 | 0.089530209 | | 37 | 4 |
| cg18432441 | 8.47E−10 | 3.60E−04 | −0.278109902 | 0.278109902 | loss | 0.79331356 | 0.515203659 | TUB; TUB | 37 | 11 |
| cg12561474 | 7.13E−10 | 3.03E−04 | −0.278039829 | 0.278039829 | loss | 0.969559706 | 0.691519876 | NGFR; LOC100288866 | 37 | 17 |
| cg03503642 | 7.77E−10 | 3.30E−04 | −0.277946658 | 0.277946658 | loss | 0.714554111 | 0.436607453 | COL12A1; COL12A1 | 37 | 6 |
| cg03507824 | 7.13E−10 | 3.03E−04 | −0.277937394 | 0.277937394 | loss | 0.761419447 | 0.483482053 | | 37 | 11 |
| cg03352173 | 4.97E−09 | 2.11E−03 | −0.277932698 | 0.277932698 | loss | 0.675030904 | 0.397098206 | SLC9C1; SLC9C1 | 37 | 3 |
| cg21062780 | 4.21E−09 | 1.79E−03 | −0.277911242 | 0.277911242 | loss | 0.797007519 | 0.519096276 | | 37 | 6 |
| cg08709360 | 1.19E−09 | 5.07E−04 | −0.27782079 | 0.27782079 | loss | 0.918092096 | 0.640271306 | | 37 | 1 |
| cg07031872 | 7.13E−10 | 3.03E−04 | −0.277793186 | 0.277793186 | loss | 0.644608662 | 0.366815476 | SLC24A4; SLC24A4 | 37 | 14 |
| cg13304350 | 9.23E−10 | 3.92E−04 | −0.277754046 | 0.277754046 | loss | 0.585700834 | 0.307946788 | | 37 | 17 |
| cg19319928 | 1.19E−09 | 5.07E−04 | −0.277728861 | 0.277728861 | loss | 0.872159449 | 0.594430588 | | 37 | 1 |
| cg24860562 | 3.67E−08 | 1.56E−02 | −0.277592931 | 0.277592931 | loss | 0.804444249 | 0.526851318 | LOC283710 | 37 | 15 |
| cg01215511 | 1.01E−09 | 4.27E−04 | −0.277548086 | 0.277548086 | loss | 0.670081857 | 0.392533771 | FLYWCH1; FLYWCH1 | 37 | 16 |
| cg01713438 | 1.21E−09 | 5.15E−04 | −0.277534918 | 0.277534918 | loss | 0.476997031 | 0.199462113 | TNN | 37 | 1 |
| cg00931742 | 4.21E−09 | 1.79E−03 | −0.277528349 | 0.277528349 | loss | 0.772515149 | 0.4949868 | MOS | 37 | 8 |
| cg23887948 | 7.13E−10 | 3.03E−04 | −0.277446845 | 0.277446845 | loss | 0.895720457 | 0.618273612 | STRA6; STRA6; STRA6; STRA6; | 37 | 15 |
| | | | | | | | | STRA6; STRA6; STRA6; STRA6 | | |
| cg16244664 | 7.13E−10 | 3.03E−04 | −0.277403766 | 0.277403766 | loss | 0.899371219 | 0.621967453 | PIWIL1; PIWIL1 | 37 | 12 |
| cg12180613 | 7.13E−10 | 3.03E−04 | −0.277347254 | 0.277347254 | loss | 0.633742313 | 0.356395059 | SHROOM1; SHROOM1 | 37 | 5 |
| cg22190721 | 7.13E−10 | 3.03E−04 | −0.277205944 | 0.277205944 | loss | 0.884611121 | 0.607405176 | ESRP2 | 37 | 16 |
| cg25017403 | 1.32E−08 | 5.59E−03 | −0.277168194 | 0.277168194 | loss | 0.784854358 | 0.507686165 | LOC283710 | 37 | 15 |
| cg14206172 | 7.13E−10 | 3.03E−04 | −0.277146021 | 0.277146021 | loss | 0.327970611 | 0.05082459 | FBXO17; FBXO17 | 37 | 19 |
| cg20347666 | 1.99E−09 | 8.43E−04 | −0.27703787 | 0.27703787 | loss | 0.750844311 | 0.473806441 | DGKI | 37 | 7 |
| cg05231970 | 7.13E−10 | 3.03E−04 | −0.277029774 | 0.277029774 | loss | 0.890662015 | 0.613632241 | | 37 | 10 |
| cg10973146 | 7.13E−10 | 3.03E−04 | −0.277024059 | 0.277024059 | loss | 0.822540494 | 0.545516435 | EXOC3L4 | 37 | 14 |
| cg00653460 | 7.13E−10 | 3.03E−04 | −0.276987806 | 0.276987806 | loss | 0.488285947 | 0.211298141 | RPL24 | 37 | 3 |
| cg27263741 | 7.13E−10 | 3.03E−04 | −0.2768827 | 0.2768827 | loss | 0.355630919 | 0.078748219 | LINC00111 | 37 | 21 |
| cg19763471 | 7.13E−10 | 3.03E−04 | −0.276872794 | 0.276872794 | loss | 0.886631306 | 0.609758512 | CACNA1G; CACNA1G; CACNA1G; | 37 | 17 |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G | | |
| cg09188304 | 7.84E−10 | 3.33E−04 | −0.276849476 | 0.276849476 | loss | 0.864484617 | 0.587635141 | CRISPLD2 | 37 | 16 |
| cg16725948 | 7.13E−10 | 3.03E−04 | −0.276833454 | 0.276833454 | loss | 0.590505366 | 0.313671912 | | 37 | 14 |
| cg03324464 | 5.85E−09 | 2.49E−03 | −0.27682049 | 0.27682049 | loss | 0.676512549 | 0.399692059 | | 37 | 1 |
| cg01841471 | 7.13E−10 | 3.03E−04 | −0.276818299 | 0.276818299 | loss | 0.640899028 | 0.364080729 | | 37 | 13 |
| cg07769957 | 2.35E−09 | 9.98E−04 | −0.276766281 | 0.276766281 | loss | 0.467214266 | 0.190447985 | NOVA2 | 37 | 19 |
| cg02104434 | 4.21E−09 | 1.79E−03 | −0.276699754 | 0.276699754 | loss | 0.793692072 | 0.516992318 | GCSAML; GCSAML; GCSAML; | 37 | 1 |
| | | | | | | | | GCSAML; GCSAML; GCSAML; | | |
| | | | | | | | | OR2C3; GCSAML- | | |
| | | | | | | | | AS1 | | |
| cg21606036 | 8.47E−10 | 3.60E−04 | −0.276696008 | 0.276696008 | loss | 0.733729549 | 0.457033541 | CMYA5 | 37 | 5 |
| cg08732750 | 7.13E−10 | 3.03E−04 | −0.276690115 | 0.276690115 | loss | 0.497696568 | 0.221006453 | LOC339524; LOC339524; LOC339524; | 37 | 1 |
| | | | | | | | | LOC339524; LOC339524 | | |
| cg07017454 | 7.84E−10 | 3.33E−04 | −0.276670366 | 0.276670366 | loss | 0.924624619 | 0.647954253 | | 37 | 1 |
| cg06776201 | 7.13E−10 | 3.03E−04 | −0.276650581 | 0.276650581 | loss | 0.971319728 | 0.694669147 | DPF3; DPF3; DPF3; DPF3 | 37 | 14 |
| cg14264194 | 7.13E−10 | 3.03E−04 | −0.276646804 | 0.276646804 | loss | 0.294110219 | 0.017463415 | RD3; RD3 | 37 | 1 |
| cg02704502 | 7.13E−10 | 3.03E−04 | −0.276642975 | 0.276642975 | loss | 0.587740487 | 0.311097512 | | 37 | 11 |
| cg16519668 | 7.13E−10 | 3.03E−04 | −0.276595908 | 0.276595908 | loss | 0.748843855 | 0.472247947 | BCAR1; BCAR1; BCAR1; BCAR1; | 37 | 16 |
| | | | | | | | | BCAR1; BCAR1; BCAR1; | | |
| | | | | | | | | BCAR1 | | |
| cg03222066 | 7.13E−10 | 3.03E−04 | −0.276590264 | 0.276590264 | loss | 0.560305475 | 0.283715212 | ABCG4; ABCG4 | 37 | 11 |
| cg23270113 | 7.84E−10 | 3.33E−04 | −0.276566541 | 0.276566541 | loss | 0.436080221 | 0.15951368 | APOC1 | 37 | 19 |
| cg08670906 | 7.13E−10 | 3.03E−04 | −0.276513431 | 0.276513431 | loss | 0.485957719 | 0.209444288 | | 37 | 6 |
| cg10955566 | 1.54E−09 | 6.54E−04 | −0.27648942 | 0.27648942 | loss | 0.539458091 | 0.562968671 | | 37 | 1 |
| cg03554573 | 7.77E−10 | 3.30E−04 | −0.276447612 | 0.276447612 | loss | 0.5206524 | 0.244204788 | C11orf53 | 37 | 11 |
| cg11052143 | 7.13E−10 | 3.03E−04 | −0.276330204 | 0.276330204 | loss | 0.586455998 | 0.310125794 | ALS2CR11; ALS2CR11; ALS2CR11; | 37 | 2 |
| | | | | | | | | ALS2CR11; ALS2CR11; | | |
| | | | | | | | | ALS2CR11; ALS2CR11; | | |
| | | | | | | | | ALS2CR11 | | |
| cg26935536 | 7.13E−10 | 3.03E−04 | −0.27631769 | 0.27631769 | loss | 0.930304202 | 0.653986512 | | 37 | 5 |
| cg20344448 | 8.11E−09 | 3.44E−03 | −0.27630098 | 0.27630098 | loss | 0.777802668 | 0.501501688 | FRMD4A | 37 | 10 |
| cg11743829 | 9.23E−10 | 3.92E−04 | −0.27627805 | 0.27627805 | loss | 0.570246174 | 0.293968124 | NBPF10; LOC100288142; | 37 | 1 |
| | | | | | | | | CD160; CD160 | | |
| cg27427581 | 7.13E−10 | 3.03E−04 | −0.276235878 | 0.276235878 | loss | 0.646449002 | 0.370213124 | | 37 | 10 |
| cg15445037 | 5.84E−08 | 2.48E−02 | −0.276217539 | 0.276217539 | loss | 0.939337051 | 0.663119512 | CDH4; CDH4; CDH4 | 37 | 20 |
| cg24490133 | 1.30E−09 | 5.52E−04 | −0.276188855 | 0.276188855 | loss | 0.706280908 | 0.430092053 | KIF26B | 37 | 1 |
| cg17445155 | 3.47E−09 | 1.47E−03 | −0.276186972 | 0.276186972 | loss | 0.826925828 | 0.550738856 | PPEF2 | 37 | 4 |
| cg08158233 | 1.19E−09 | 5.07E−04 | −0.276181663 | 0.276181663 | loss | 0.499236287 | 0.223054624 | | 37 | 8 |
| cg01471372 | 7.13E−10 | 3.03E−04 | −0.276151829 | 0.276151829 | loss | 0.765347964 | 0.489196135 | TACC1 | 37 | 8 |
| cg18507732 | 7.13E−10 | 3.03E−04 | −0.276142546 | 0.276142546 | loss | 0.52732597 | 0.251183424 | | 37 | 14 |
| cg23698536 | 7.13E−10 | 3.03E−04 | −0.276087373 | 0.276087373 | loss | 0.898770385 | 0.622683012 | FAM83A; FAM83A | 37 | 8 |
| cg23012310 | 1.21E−09 | 5.15E−04 | −0.276086707 | 0.276086707 | loss | 0.854416265 | 0.578329559 | | 37 | 14 |
| cg21201393 | 1.54E−08 | 6.56E−03 | −0.276073945 | 0.276073945 | loss | 0.902201709 | 0.626127765 | EGFLAM; EGFLAM; EGFLAM; | 37 | 5 |
| | | | | | | | | EGFLAM | | |
| cg12443604 | 7.13E−10 | 3.03E−04 | −0.276056227 | 0.276056227 | loss | 0.516191121 | 0.240134894 | BTBD17; BTBD17 | 37 | 17 |
| cg19273694 | 7.13E−10 | 3.03E−04 | −0.276010958 | 0.276010958 | loss | 0.791259128 | 0.515248171 | MFSD2B | 37 | 2 |
| cg15903282 | 7.13E−10 | 3.03E−04 | −0.27600268 | 0.27600268 | loss | 0.795188757 | 0.519186076 | EPHB1 | 37 | 3 |
| cg15804973 | 7.13E−10 | 3.03E−04 | −0.275973208 | 0.275973208 | loss | 0.512435996 | 0.236462788 | MAP3K5 | 37 | 6 |
| cg07064495 | 7.13E−10 | 3.03E−04 | −0.275961733 | 0.275961733 | loss | 0.302789489 | 0.026827756 | | 37 | 18 |
| cg17210938 | 8.80E−09 | 3.74E−03 | −0.275947283 | 0.275947283 | loss | 0.406532815 | 0.130585532 | TACSTD2; TACSTD2 | 37 | 1 |
| cg05796704 | 7.13E−10 | 3.03E−04 | −0.275891085 | 0.275891085 | loss | 0.661525391 | 0.385634306 | FBXO44; FBXO2; FBXO44; FBXO44; | 37 | 1 |
| | | | | | | | | FBXO44 | | |
| cg17103155 | 7.13E−10 | 3.03E−04 | −0.275841659 | 0.275841659 | loss | 0.727090606 | 0.451248947 | | 37 | 4 |
| cg05139523 | 5.85E−09 | 2.49E−03 | −0.275798794 | 0.275798794 | loss | 0.6946653 | 0.418866506 | RIMS4; RIMS4 | 37 | 20 |
| cg15823502 | 7.13E−10 | 3.03E−04 | −0.275796334 | 0.275796334 | loss | 0.429819147 | 0.154022814 | | 37 | 6 |
| cg00138926 | 1.19E−09 | 5.07E−04 | −0.275752259 | 0.275752259 | loss | 0.879515053 | 0.603762794 | | 37 | 2 |
| cg05241134 | 7.48E−09 | 3.18E−03 | −0.275713044 | 0.275713044 | loss | 0.586310074 | 0.310597029 | MYOM2 | 37 | 8 |
| cg17397004 | 7.13E−10 | 3.03E−04 | −0.275697753 | 0.275697753 | loss | 0.6608857 | 0.385187947 | KDELR3; KDELR3 | 37 | 22 |
| cg25122629 | 8.47E−10 | 3.60E−04 | −0.275610864 | 0.275610864 | loss | 0.389949704 | 0.114338839 | | 37 | 2 |
| cg22414192 | 5.59E−09 | 2.37E−03 | −0.275585216 | 0.275585216 | loss | 0.91201891 | 0.636433693 | KCNG1 | 37 | 20 |
| cg24317190 | 7.77E−10 | 3.30E−04 | −0.275581974 | 0.275581974 | loss | 0.940856509 | 0.665274535 | | 37 | 7 |
| cg23255283 | 1.22E−08 | 5.16E−03 | −0.275502927 | 0.275502927 | loss | 0.608257362 | 0.332754435 | LINC00908 | 37 | 18 |
| cg25313468 | 8.80E−09 | 3.74E−03 | −0.275468174 | 0.275468174 | loss | 0.589745274 | 0.3142771 | REST | 37 | 4 |
| cg14181528 | 7.13E−10 | 3.03E−04 | −0.2754516 | 0.2754516 | loss | 0.544743 | 0.2692914 | | 37 | 1 |
| cg20038187 | 7.13E−10 | 3.03E−04 | −0.27544109 | 0.27544109 | loss | 0.804858043 | 0.529416953 | | 37 | 10 |
| cg11936341 | 7.13E−10 | 3.03E−04 | −0.275417167 | 0.275417167 | loss | 0.532325038 | 0.256907871 | | 37 | 6 |
| cg04098547 | 5.84E−08 | 2.48E−02 | −0.275409586 | 0.275409586 | loss | 0.739319257 | 0.463909671 | ABCC12 | 37 | 16 |
| cg11871991 | 8.47E−10 | 3.60E−04 | −0.275401894 | 0.275401894 | loss | 0.590959594 | 0.3155577 | CPEB1; CPEB1; CPEB1; CPEB1 | 37 | 15 |
| cg24413781 | 7.13E−10 | 3.03E−04 | −0.275372764 | 0.275372764 | loss | 0.299046511 | 0.023673748 | CERS4 | 37 | 19 |
| cg23894948 | 7.13E−10 | 3.03E−04 | −0.275331949 | 0.275331949 | loss | 0.755773043 | 0.480441094 | SLC7A4 | 37 | 22 |
| cg00232387 | 1.42E−09 | 6.01E−04 | −0.275315304 | 0.275315304 | loss | 0.758972609 | 0.483657306 | DIRAS1 | 37 | 19 |
| cg12099459 | 7.13E−10 | 3.03E−04 | −0.275311175 | 0.275311175 | loss | 0.631812998 | 0.356501824 | NBPF8; NBPF8; NBPF8 | 37 | 1 |
| cg06502392 | 1.30E−09 | 5.52E−04 | −0.27529334 | 0.27529334 | loss | 0.81777644 | 0.5424831 | | 37 | 3 |
| cg26027576 | 7.13E−10 | 3.03E−04 | −0.275290012 | 0.275290012 | loss | 0.666941983 | 0.391651971 | | 37 | 8 |
| cg14299480 | 3.57E−09 | 1.52E−03 | −0.275247061 | 0.275247061 | loss | 0.6555192 | 0.380272139 | GGN | 37 | 19 |
| cg17371020 | 1.99E−09 | 8.43E−04 | −0.275191699 | 0.275191699 | loss | 0.767308075 | 0.492116376 | TRIM2; TRIM2 | 37 | 4 |
| cg10153349 | 1.42E−09 | 6.01E−04 | −0.275124286 | 0.275124286 | loss | 0.643221398 | 0.368097112 | SPON1 | 37 | 11 |
| cg05404792 | 7.77E−10 | 3.30E−04 | −0.275122267 | 0.275122267 | loss | 0.839616449 | 0.564494182 | MT1L | 37 | 16 |
| cg12103951 | 3.88E−09 | 1.65E−03 | −0.275107775 | 0.275107775 | loss | 0.585954628 | 0.310846853 | | 37 | 10 |
| cg24750156 | 7.13E−10 | 3.03E−04 | −0.275107458 | 0.275107458 | loss | 0.666303258 | 0.3911958 | B4GALNT1; B4GALNT1; B4GALNT1 | 37 | 12 |
| cg14576824 | 7.13E−10 | 3.03E−04 | −0.275054202 | 0.275054202 | loss | 0.475211643 | 0.200157441 | RPS6KC1; RPS6KC1 | 37 | 1 |
| cg13251842 | 1.42E−09 | 6.01E−04 | −0.275011398 | 0.275011398 | loss | 0.928509051 | 0.653497653 | MIRLET7BHG; MIRLET7A3; | 37 | 22 |
| | | | | | | | | MIRLET7B; MIR4763 | | |
| cg19628934 | 7.13E−10 | 3.03E−04 | −0.274976286 | 0.274976286 | loss | 0.459688904 | 0.184712618 | | 37 | 14 |
| cg19154323 | 8.80E−09 | 3.74E−03 | −0.274898473 | 0.274898473 | loss | 0.606550926 | 0.331652453 | PLEKHM3 | 37 | 2 |
| cg15392457 | 7.13E−10 | 3.03E−04 | −0.27489732 | 0.27489732 | loss | 0.736207179 | 0.461309859 | | 37 | 11 |
| cg03694403 | 7.13E−10 | 3.03E−04 | −0.274881988 | 0.274881988 | loss | 0.854770553 | 0.579888565 | | 37 | 8 |
| cg05204532 | 7.13E−10 | 3.03E−04 | −0.274868789 | 0.274868789 | loss | 0.488356683 | 0.213487894 | | 37 | 2 |
| cg15600051 | 1.10E−09 | 4.65E−04 | −0.274849217 | 0.274849217 | loss | 0.792573028 | 0.517723812 | ZFYVE28; ZFYVE28; ZFYVE28; | 37 | 4 |
| | | | | | | | | ZFYVE28; ZFYVE28; ZFYVE28 | | |
| cg01711322 | 7.13E−10 | 3.03E−04 | −0.27483254 | 0.27483254 | loss | 0.78984494 | 0.5150124 | GPR25 | 37 | 1 |
| cg09953520 | 7.48E−09 | 3.18E−03 | −0.274811598 | 0.274811598 | loss | 0.534903104 | 0.260091506 | EBF2 | 37 | 8 |
| cg11705954 | 7.77E−10 | 3.30E−04 | −0.274718688 | 0.274718688 | loss | 0.938633747 | 0.663915059 | | 37 | 1 |
| cg26665274 | 8.47E−10 | 3.60E−04 | −0.274716782 | 0.274716782 | loss | 0.863225417 | 0.588508635 | BMP2 | 37 | 20 |
| cg09546802 | 7.13E−10 | 3.03E−04 | −0.274694562 | 0.274694562 | loss | 0.406328258 | 0.131633696 | PRELID2; PRELID2; PRELID2 | 37 | 5 |
| cg24377495 | 7.77E−10 | 3.30E−04 | −0.274630814 | 0.274630814 | loss | 0.684131038 | 0.409500224 | | 37 | 19 |
| cg06181761 | 7.13E−10 | 3.03E−04 | −0.274625527 | 0.274625527 | loss | 0.853324851 | 0.578699324 | | 37 | 14 |
| cg20794351 | 7.13E−10 | 3.03E−04 | −0.274579984 | 0.274579984 | loss | 0.869668166 | 0.595088182 | | 37 | 8 |
| cg17149030 | 1.01E−09 | 4.27E−04 | −0.274576587 | 0.274576587 | loss | 0.70472327 | 0.430146682 | PGPEP1L; PGPEP1L; PGPEP1L | 37 | 15 |
| cg23829948 | 1.10E−09 | 4.65E−04 | −0.27457389 | 0.27457389 | loss | 0.867706313 | 0.593132424 | | 37 | 4 |
| cg18623649 | 7.13E−10 | 3.03E−04 | −0.274561619 | 0.274561619 | loss | 0.750806813 | 0.476245194 | | 37 | 10 |
| cg05156120 | 8.47E−10 | 3.60E−04 | −0.274538688 | 0.274538688 | loss | 0.871502053 | 0.596963365 | MEOX1; MEOX1; MEOX1 | 37 | 17 |
| cg02630888 | 7.84E−10 | 3.33E−04 | −0.274519178 | 0.274519178 | loss | 0.838845142 | 0.564325965 | FBXL5; FBXL5; FBXL5; FBXL5 | 37 | 4 |
| cg09320662 | 7.77E−10 | 3.30E−04 | −0.274500279 | 0.274500279 | loss | 0.432372206 | 0.157871926 | LRCOL1 | 37 | 12 |
| cg01973920 | 7.13E−10 | 3.03E−04 | −0.274495202 | 0.274495202 | loss | 0.605826808 | 0.331331606 | | 37 | 5 |
| cg12585923 | 7.13E−10 | 3.03E−04 | −0.274482045 | 0.274482045 | loss | 0.489128915 | 0.214646871 | CCDC152 | 37 | 5 |
| cg00567703 | 7.13E−10 | 3.03E−04 | −0.274460466 | 0.274460466 | loss | 0.418584747 | 0.144124281 | HOXC4; HOXC6; HOXC5 | 37 | 12 |
| cg12589798 | 7.13E−10 | 3.03E−04 | −0.27432216 | 0.27432216 | loss | 0.81861193 | 0.544289771 | CDH22 | 37 | 20 |
| cg09269945 | 7.13E−10 | 3.03E−04 | −0.274302164 | 0.274302164 | loss | 0.532280964 | 0.2579788 | BMP7 | 37 | 20 |
| cg21450652 | 8.47E−10 | 3.60E−04 | −0.2742846 | 0.2742846 | loss | 0.850417906 | 0.576133306 | PRSS42; PRSS42 | 37 | 3 |
| cg17142470 | 7.13E−10 | 3.03E−04 | −0.274283678 | 0.274283678 | loss | 0.370611674 | 0.096327995 | SORBS3 | 37 | 8 |
| cg21207436 | 1.54E−09 | 6.54E−04 | −0.274256194 | 0.274256194 | loss | 0.833161642 | 0.558905447 | VRTN | 37 | 14 |
| cg17205324 | 7.13E−10 | 3.03E−04 | −0.274245111 | 0.274245111 | loss | 0.427220006 | 0.152974895 | EFS; EFS; EFS | 37 | 14 |
| cg02470697 | 7.13E−10 | 3.03E−04 | −0.274237692 | 0.274237692 | loss | 0.877547398 | 0.603309706 | GTF2A1L; STON1- | 37 | 2 |
| | | | | | | | | GTF2A1L; STON1- | | |
| | | | | | | | | GTF2A1L; GTF2A1L; STON1- | | |
| | | | | | | | | GTF2A1L | | |
| cg11693285 | 1.01E−09 | 4.27E−04 | −0.27423363 | 0.27423363 | loss | 0.320926994 | 0.046693364 | | 37 | 10 |
| cg17780447 | 7.13E−10 | 3.03E−04 | −0.274195265 | 0.274195265 | loss | 0.622134583 | 0.347939318 | | 37 | 19 |
| cg08043020 | 1.01E−09 | 4.27E−04 | −0.274191898 | 0.274191898 | loss | 0.628521362 | 0.354329465 | | 37 | 11 |
| cg07618085 | 7.77E−10 | 3.30E−04 | −0.274189166 | 0.274189166 | loss | 0.682832355 | 0.408643188 | TBC1D16; TBC1D16; TBC1D16; | 37 | 17 |
| | | | | | | | | TBC1D16; TBC1D16; TBC1D16; | | |
| | | | | | | | | TBC1D16 | | |
| cg26023303 | 1.73E−09 | 7.34E−04 | −0.274155645 | 0.274155645 | loss | 0.481776632 | 0.207620988 | | 37 | 14 |
| cg14166701 | 7.13E−10 | 3.03E−04 | −0.274137392 | 0.274137392 | loss | 0.899050804 | 0.624913412 | ASPG | 37 | 14 |
| cg15255946 | 1.99E−09 | 8.43E−04 | −0.274134567 | 0.274134567 | loss | 0.741121691 | 0.466987124 | HLA-DQB2 | 37 | 6 |
| cg27423692 | 7.13E−10 | 3.03E−04 | −0.273974813 | 0.273974813 | loss | 0.87359326 | 0.599618447 | | 37 | 10 |
| cg22910549 | 7.13E−10 | 3.03E−04 | −0.273892703 | 0.273892703 | loss | 0.871282068 | 0.597389365 | KLHDC7B | 37 | 22 |
| cg25718438 | 7.77E−10 | 3.30E−04 | −0.273886762 | 0.273886762 | loss | 0.624374526 | 0.350487765 | WFDC5; WFDC5 | 37 | 20 |
| cg16003238 | 7.13E−10 | 3.03E−04 | −0.273859743 | 0.273859743 | loss | 0.641742813 | 0.367883071 | IGDCC3 | 37 | 15 |
| cg22268510 | 7.13E−10 | 3.03E−04 | −0.273774574 | 0.273774574 | loss | 0.30811673 | 0.034342156 | PRRT1 | 37 | 6 |
| cg02834447 | 7.77E−10 | 3.30E−04 | −0.273738984 | 0.273738984 | loss | 0.829183419 | 0.555444435 | | 37 | 2 |
| Cg10249734 | 7.13E−10 | 3.03E−04 | −0.273729992 | 0.273729992 | loss | 0.744534415 | 0.470804424 | SECTM1; SECTM1 | 37 | 17 |
| cg01717649 | 7.13E−10 | 3.03E−04 | −0.273693077 | 0.273693077 | loss | 0.688469025 | 0.414775947 | | 37 | 11 |
| cg04612959 | 1.87E−09 | 7.93E−04 | −0.273682416 | 0.273682416 | loss | 0.671616763 | 0.397934347 | SDK1; SDK1 | 37 | 7 |
| cg18414021 | 1.10E−09 | 4.65E−04 | −0.273636237 | 0.273636237 | loss | 0.440040606 | 0.166404368 | CCDC85C | 37 | 14 |
| cg21697667 | 7.13E−10 | 3.03E−04 | −0.27360758 | 0.27360758 | loss | 0.777956362 | 0.504348782 | | 37 | 2 |
| cg19042287 | 1.01E−09 | 4.27E−04 | −0.273516261 | 0.273516261 | loss | 0.660735438 | 0.387219176 | NR2E1; NR2E1 | 37 | 6 |
| cg25573884 | 7.13E−10 | 3.03E−04 | −0.273481449 | 0.273481449 | loss | 0.914908596 | 0.641427147 | | 37 | 7 |
| cg10081626 | 7.13E−10 | 3.03E−04 | −0.27342111 | 0.27342111 | loss | 0.63313974 | 0.359718629 | | 37 | 10 |
| cg27478242 | 9.23E−10 | 3.92E−04 | −0.273395196 | 0.273395196 | loss | 0.446776713 | 0.173381517 | | 37 | 12 |
| cg08846011 | 1.01E−09 | 4.27E−04 | −0.27338597 | 0.27338597 | loss | 0.868900417 | 0.595514447 | | 37 | 1 |
| cg00987015 | 1.30E−09 | 5.52E−04 | −0.273384958 | 0.273384958 | loss | 0.824223958 | 0.550839 | CHRM1 | 37 | 11 |
| cg13928759 | 7.13E−10 | 3.03E−04 | −0.273369778 | 0.273369778 | loss | 0.66265416 | 0.389284382 | LGI1 | 37 | 10 |
| cg14328523 | 7.13E−10 | 3.03E−04 | −0.273361358 | 0.273361358 | loss | 0.379987385 | 0.106626026 | | 37 | 9 |
| cg26451735 | 8.55E−08 | 3.63E−02 | −0.273329777 | 0.273329777 | loss | 0.518898994 | 0.245569218 | STRA6; STRA6; STRA6; STRA6; | 37 | 15 |
| | | | | | | | | STRA6; STRA6; STRA6; STRA6 | | |
| cg01355876 | 7.13E−10 | 3.03E−04 | −0.273273571 | 0.273273571 | loss | 0.88276103 | 0.609487459 | LOC728637; LOC728637 | 37 | 5 |
| cg10772086 | 1.19E−09 | 5.07E−04 | −0.273254372 | 0.273254372 | loss | 0.696180213 | 0.422925841 | TF | 37 | 3 |
| cg19706390 | 7.77E−10 | 3.30E−04 | −0.273254099 | 0.273254099 | loss | 0.807299417 | 0.534045318 | CASZ1; CASZ1 | 37 | 1 |
| cg26758396 | 7.13E−10 | 3.03E−04 | −0.273219395 | 0.273219395 | loss | 0.652905036 | 0.379685641 | FERMT1; FERMT1 | 37 | 20 |
| cg13326914 | 7.48E−09 | 3.18E−03 | −0.273217448 | 0.273217448 | loss | 0.807242965 | 0.534025518 | LRRC66 | 37 | 4 |
| cg01054354 | 1.42E−09 | 6.01E−04 | −0.273183773 | 0.273183773 | loss | 0.590845491 | 0.317661718 | LYSMD4; LYSMD4; LY5MD4; | 37 | 15 |
| | | | | | | | | LYSMD4; LYSMD4; LYSMD4; | | |
| | | | | | | | | LYSMD4 | | |
| cg02389040 | 5.01E−08 | 2.13E−02 | −0.273179029 | 0.273179029 | loss | 0.594527606 | 0.321348576 | | 37 | 6 |
| cg14924160 | 7.13E−10 | 3.03E−04 | −0.273173885 | 0.273173885 | loss | 0.842693932 | 0.569520047 | SUGT1P3; TPTE2P5; TPTE2P5 | 37 | 13 |
| cg10668414 | 7.13E−10 | 3.03E−04 | −0.273170804 | 0.273170804 | loss | 0.504697751 | 0.231526947 | | 37 | 20 |
| cg11786623 | 1.54E−08 | 6.56E−03 | −0.273148843 | 0.273148843 | loss | 0.424409817 | 0.151260974 | | 37 | 5 |
| cg21122199 | 7.13E−10 | 3.03E−04 | −0.273148576 | 0.273148576 | loss | 0.575269323 | 0.302120747 | C17orf64 | 37 | 17 |
| cg21401642 | 7.13E−10 | 3.03E−04 | −0.273120182 | 0.273120182 | loss | 0.73703027 | 0.463910088 | | 37 | 4 |
| cg02841651 | 7.13E−10 | 3.03E−04 | −0.273103135 | 0.273103135 | loss | 0.690413206 | 0.417310071 | | 37 | 11 |
| cg09357462 | 7.13E−10 | 3.03E−04 | −0.273100502 | 0.273100502 | loss | 0.68805826 | 0.414957759 | DUSP5 | 37 | 10 |
| cg04466022 | 1.03E−08 | 4.39E−03 | −0.273094904 | 0.273094904 | loss | 0.940987709 | 0.667892806 | MDGA1 | 37 | 6 |
| cg20770435 | 8.47E−10 | 3.60E−04 | −0.273087979 | 0.273087979 | loss | 0.514017796 | 0.240929818 | CELF4; CELF4; CELF4; CELF4 | 37 | 18 |
| cg10372829 | 7.13E−10 | 3.03E−04 | −0.273075424 | 0.273075424 | loss | 0.676394936 | 0.403319512 | | 37 | 13 |
| cg07634645 | 7.13E−10 | 3.03E−04 | −0.273035427 | 0.273035427 | loss | 0.445200004 | 0.172164576 | PLBD1; PLBD1 | 37 | 12 |
| cg01423389 | 7.13E−10 | 3.03E−04 | −0.273024585 | 0.273024585 | loss | 0.743043885 | 0.4700193 | | 37 | 3 |
| cg12288726 | 7.13E−10 | 3.03E−04 | −0.272965671 | 0.272965671 | loss | 0.834535 | 0.561569329 | ARF5 | 37 | 7 |
| cg16654603 | 1.42E−09 | 6.01E−04 | −0.272962731 | 0.272962731 | loss | 0.624380572 | 0.351417841 | | 37 | 17 |
| cg01065977 | 1.19E−09 | 5.07E−04 | −0.272928263 | 0.272928263 | loss | 0.879463392 | 0.606535129 | ISYNA1; ISYNA1; ISYNA1; ISYNA1 | 37 | 19 |
| cg27560452 | 1.02E−09 | 4.33E−04 | −0.272920055 | 0.272920055 | loss | 0.73436899 | 0.461448935 | | 37 | 16 |
| cg22570042 | 1.68E−09 | 7.12E−04 | −0.272906779 | 0.272906779 | loss | 0.726753232 | 0.453846453 | CCDC144NL; CCDC144NL; | 37 | 17 |
| | | | | | | | | LOC440416 | | |
| cg27315996 | 8.47E−10 | 3.60E−04 | −0.27290249 | 0.27290249 | loss | 0.869487702 | 0.596585212 | DMTN; DMTN; DMTN; DMTN; | 37 | 8 |
| | | | | | | | | DMTN; DMTN | | |
| cg15656462 | 7.13E−10 | 3.03E−04 | −0.272890819 | 0.272890819 | loss | 0.870240108 | 0.597349288 | | 37 | 10 |
| cg20167366 | 1.96E−08 | 8.33E−03 | −0.272872872 | 0.272872872 | loss | 0.549612372 | 0.2767395 | | 37 | 6 |
| cg17178468 | 7.13E−10 | 3.03E−04 | −0.272864116 | 0.272864116 | loss | 0.404989172 | 0.132125056 | | 37 | 17 |
| cg05349837 | 7.13E−10 | 3.03E−04 | −0.272823225 | 0.272823225 | loss | 0.828980919 | 0.556157694 | HOXC4 | 37 | 12 |
| cg07044859 | 6.89E−09 | 2.93E−03 | −0.272781142 | 0.272781142 | loss | 0.561286872 | 0.288505729 | | 37 | 10 |
| cg13521077 | 5.41E−08 | 2.30E−02 | −0.272765727 | 0.272765727 | loss | 0.689818051 | 0.417052324 | C4orf22; C4orf22 | 37 | 4 |
| cg16619576 | 1.12E−08 | 4.76E−03 | −0.272764666 | 0.272764666 | loss | 0.704432555 | 0.431667888 | | 37 | 2 |
| cg08317694 | 7.13E−10 | 3.03E−04 | −0.272690993 | 0.272690993 | loss | 0.497727075 | 0.225036082 | CRABP1 | 37 | 15 |
| cg04661001 | 7.84E−10 | 3.33E−04 | −0.272663674 | 0.272663674 | loss | 0.909225515 | 0.636561841 | SLC25A42 | 37 | 19 |
| cg25283465 | 7.13E−10 | 3.03E−04 | −0.272657332 | 0.272657332 | loss | 0.371346628 | 0.098689296 | | 37 | 1 |
| cg03483626 | 7.13E−10 | 3.03E−04 | −0.272598263 | 0.272598263 | loss | 0.54492444 | 0.272326176 | KCNA3 | 37 | 1 |
| cg16448636 | 7.13E−10 | 3.03E−04 | −0.272586195 | 0.272586195 | loss | 0.573771189 | 0.301184994 | | 37 | 7 |
| cg14044120 | 7.13E−10 | 3.03E−04 | −0.27249009 | 0.27249009 | loss | 0.825719655 | 0.553229565 | ENTPD2; ENTPD2 | 37 | 9 |
| cg12343363 | 1.96E−08 | 8.33E−03 | −0.272445901 | 0.272445901 | loss | 0.936950672 | 0.664504771 | METTL21C; METTL21C | 37 | 13 |
| cg19413350 | 1.54E−09 | 6.54E−04 | −0.272427999 | 0.272427999 | loss | 0.806518281 | 0.534090282 | | 37 | 8 |
| cg03257743 | 1.68E−09 | 7.12E−04 | −0.272376198 | 0.272376198 | loss | 0.719759051 | 0.447392853 | | 37 | 2 |
| cg15750303 | 3.78E−09 | 1.61E−03 | −0.272330585 | 0.272330585 | loss | 0.861480332 | 0.589149747 | NTSC1B; NTSC1B; NTSC1B; | 37 | 2 |
| | | | | | | | | NTSC1B; NTSC1B- | | |
| | | | | | | | | RDH14; NTSC1B- | | |
| | | | | | | | | RDH14; NTSC1B | | |
| cg08439880 | 1.99E−09 | 8.43E−04 | −0.272317888 | 0.272317888 | loss | 0.815223506 | 0.542905618 | SRPRB | 37 | 3 |
| cg17160439 | 7.13E−10 | 3.03E−04 | −0.272304881 | 0.272304881 | loss | 0.690634534 | 0.418329653 | NAALADL1 | 37 | 11 |
| cg06908857 | 8.47E−10 | 3.60E−04 | −0.272230302 | 0.272230302 | loss | 0.787552849 | 0.515322547 | | 37 | 16 |
| cg01937803 | 7.13E−10 | 3.03E−04 | −0.272216066 | 0.272216066 | loss | 0.402119017 | 0.129902951 | | 37 | 14 |
| cg21511948 | 7.13E−10 | 3.03E−04 | −0.272215724 | 0.272215724 | loss | 0.629197694 | 0.356981971 | ELFN2 | 37 | 22 |
| cg06355129 | 7.13E−10 | 3.03E−04 | −0.272207899 | 0.272207899 | loss | 0.386405374 | 0.114197475 | NES | 37 | 1 |
| cg24973864 | 7.77E−10 | 3.30E−04 | −0.272187062 | 0.272187062 | loss | 0.868186274 | 0.595999212 | | 37 | 5 |
| cg11515089 | 7.13E−10 | 3.03E−04 | −0.272057539 | 0.272057539 | loss | 0.850526015 | 0.578468476 | | 37 | 17 |
| cg08837626 | 4.57E−09 | 1.94E−03 | −0.271986276 | 0.271986276 | loss | 0.6542285 | 0.382242224 | | 37 | 7 |
| cg00797104 | 1.30E−09 | 5.52E−04 | −0.271985766 | 0.271985766 | loss | 0.607533319 | 0.335547553 | | 37 | 6 |
| cg05096209 | 7.13E−10 | 3.03E−04 | −0.271916066 | 0.271916066 | loss | 0.896281613 | 0.624365547 | LOC401242 | 37 | 6 |
| cg13791841 | 7.13E−10 | 3.03E−04 | −0.271906252 | 0.271906252 | loss | 0.77563917 | 0.503732918 | PHOX2A | 37 | 11 |
| cg08048268 | 6.89E−09 | 2.93E−03 | −0.271889528 | 0.271889528 | loss | 0.533559987 | 0.261670459 | SRPRB | 37 | 3 |
| cg14482569 | 7.13E−10 | 3.03E−04 | −0.271880275 | 0.271880275 | loss | 0.313590691 | 0.041710416 | ZBTB44 | 37 | 11 |
| cg25788719 | 7.13E−10 | 3.03E−04 | −0.271874647 | 0.271874647 | loss | 0.783527658 | 0.511653012 | HSPA12B; HSPA12B | 37 | 20 |
| cg27498545 | 1.22E−08 | 5.16E−03 | −0.271823221 | 0.271823221 | loss | 0.812290304 | 0.540467082 | SOGA2 | 37 | 18 |
| cg22020227 | 7.13E−10 | 3.03E−04 | −0.271815178 | 0.271815178 | loss | 0.807259308 | 0.535444129 | OLFML3; OLFML3; OLFML3 | 37 | 1 |
| cg01795955 | 5.84E−08 | 2.48E−02 | −0.271807208 | 0.271807208 | loss | 0.759906608 | 0.4880994 | | 37 | 2 |
| cg16906964 | 8.47E−10 | 3.60E−04 | −0.271782837 | 0.271782837 | loss | 0.622708402 | 0.350925565 | | 37 | 1 |
| cg16751357 | 7.13E−10 | 3.03E−04 | −0.271757195 | 0.271757195 | loss | 0.332768834 | 0.061011639 | | 37 | 6 |
| cg19218505 | 7.13E−10 | 3.03E−04 | −0.271689474 | 0.271689474 | loss | 0.812964762 | 0.541275288 | TUB; TUB | 37 | 11 |
| cg19442201 | 7.77E−10 | 3.30E−04 | −0.271662246 | 0.271662246 | loss | 0.703403528 | 0.431741282 | | 37 | 15 |
| cg16760981 | 7.13E−10 | 3.03E−04 | −0.271636134 | 0.271636134 | loss | 0.788768423 | 0.517132288 | | 37 | 11 |
| cg13392957 | 7.13E−10 | 3.03E−04 | −0.271616986 | 0.271616986 | loss | 0.711176498 | 0.439559512 | NBPF8; NBPF8; NBPF8 | 37 | 1 |
| cg05399330 | 7.13E−10 | 3.03E−04 | −0.271578061 | 0.271578061 | loss | 0.723911138 | 0.452333076 | BSND | 37 | 1 |
| cg22196260 | 7.13E−10 | 3.03E−04 | −0.271477538 | 0.271477538 | loss | 0.349605142 | 0.078127604 | MYBPC2 | 37 | 19 |
| cg09235562 | 2.16E−09 | 9.17E−04 | −0.271466692 | 0.271466692 | loss | 0.465462345 | 0.193995654 | | 37 | 19 |
| cg10112270 | 7.13E−10 | 3.03E−04 | −0.271436802 | 0.271436802 | loss | 0.529754896 | 0.258318094 | | 37 | 1 |
| cg13953753 | 7.77E−10 | 3.30E−04 | −0.271435384 | 0.271435384 | loss | 0.567564419 | 0.296129035 | VAX1; VAX1 | 37 | 10 |
| cg16861209 | 7.13E−10 | 3.03E−04 | −0.271422051 | 0.271422051 | loss | 0.771957151 | 0.5005351 | SLC7A9; SLC7A9; SLC7A9 | 37 | 19 |
| cg10600889 | 1.10E−09 | 4.65E−04 | −0.271409447 | 0.271409447 | loss | 0.432994225 | 0.161584778 | FBXO47; FBXO47 | 37 | 17 |
| cg04178634 | 7.13E−10 | 3.03E−04 | −0.271365483 | 0.271365483 | loss | 0.915264136 | 0.643898653 | GNG7 | 37 | 19 |
| cg26187194 | 3.57E−09 | 1.52E−03 | −0.271359568 | 0.271359568 | loss | 0.843072826 | 0.571713259 | | 37 | 10 |
| cg05958582 | 1.19E−09 | 5.07E−04 | −0.2712789 | 0.2712789 | loss | 0.680936547 | 0.409657647 | PKD2L1; PKD2L1; PKD2L1; | 37 | 10 |
| | | | | | | | | PKD2L1 | | |
| cg11372818 | 1.22E−08 | 5.16E−03 | −0.271268191 | 0.271268191 | loss | 0.876223685 | 0.604955494 | | 37 | 10 |
| cg13810723 | 8.56E−10 | 3.63E−04 | −0.271245782 | 0.271245782 | loss | 0.560337212 | 0.289091429 | SRRM4 | 37 | 12 |
| cg14264372 | 7.13E−10 | 3.03E−04 | −0.271243444 | 0.271243444 | loss | 0.634565079 | 0.363321635 | | 37 | 4 |
| cg17471939 | 7.84E−10 | 3.33E−04 | −0.271232199 | 0.271232199 | loss | 0.312121727 | 0.040889528 | TP73; TP73; TP73; TP73; TP73; | 37 | 1 |
| | | | | | | | | TP73 | | |
| cg03923640 | 6.89E−09 | 2.93E−03 | −0.271226651 | 0.271226651 | loss | 0.782525398 | 0.511298747 | | 37 | 16 |
| cg14479947 | 7.13E−10 | 3.03E−04 | −0.271168485 | 0.271168485 | loss | 0.513959838 | 0.242791353 | C15orf56; PAK6; PAK6; PAK6; | 37 | 15 |
| | | | | | | | | PAK6; PAK6 | | |
| cg00166722 | 7.13E−10 | 3.03E−04 | −0.271163551 | 0.271163551 | loss | 0.943867157 | 0.672703606 | FANCD2OS; FANCD2OS; FANCD2OS; | 37 | 3 |
| | | | | | | | | FANCD2OS | | |
| cg15594585 | 7.13E−10 | 3.03E−04 | −0.271163512 | 0.271163512 | loss | 0.396601702 | 0.12543819 | | 37 | 1 |
| cg04190888 | 7.13E−10 | 3.03E−04 | −0.271157599 | 0.271157599 | loss | 0.847094975 | 0.575937376 | | 37 | 10 |
| cg17558772 | 1.68E−09 | 7.12E−04 | −0.271138114 | 0.271138114 | loss | 0.678340885 | 0.407202771 | ADARB2 | 37 | 10 |
| cg26510023 | 7.13E−10 | 3.03E−04 | −0.27111526 | 0.27111526 | loss | 0.77829213 | 0.507176871 | | 37 | 10 |
| cg23109891 | 7.13E−10 | 3.03E−04 | −0.271065035 | 0.271065035 | loss | 0.860991553 | 0.589926518 | SOX10 | 37 | 22 |
| cg10674793 | 7.13E−10 | 3.03E−04 | −0.271014711 | 0.271014711 | loss | 0.736864975 | 0.465850265 | DVL3 | 37 | 3 |
| cg26135325 | 8.11E−09 | 3.44E−03 | 0.270991632 | 0.270991632 | gain | 0.340095457 | 0.611087088 | LCE3A | 37 | 1 |
| cg14503395 | 1.96E−08 | 8.33E−03 | −0.270932936 | 0.270932936 | loss | 0.787337142 | 0.516404206 | CRB2 | 37 | 9 |
| cg27140633 | 7.13E−10 | 3.03E−04 | −0.270927448 | 0.270927448 | loss | 0.952430425 | 0.681502976 | | 37 | 11 |
| cg12872238 | 1.01E−09 | 4.27E−04 | −0.270911226 | 0.270911226 | loss | 0.532269332 | 0.261358106 | ZCCHC16; TRPC5 | 37 | X |
| cg24837256 | 7.13E−10 | 3.03E−04 | −0.27090814 | 0.27090814 | loss | 0.657670492 | 0.386762353 | IFFO2 | 37 | 1 |
| cg06066088 | 7.13E−10 | 3.03E−04 | −0.27087422 | 0.27087422 | loss | 0.654883743 | 0.384009524 | P2RX5; P2RX5; P2RX5; P2RX5; | 37 | 17 |
| | | | | | | | | P2RX5-TAX1BP3 | | |
| cg12528597 | 9.23E−10 | 3.92E−04 | −0.270859699 | 0.270859699 | loss | 0.850157887 | 0.579298188 | RTN4RL2 | 37 | 11 |
| cg22708150 | 1.30E−09 | 5.52E−04 | −0.270774489 | 0.270774489 | loss | 0.662052336 | 0.391277847 | LY6G5C | 37 | 6 |
| cg23374892 | 7.13E−10 | 3.03E−04 | −0.270732838 | 0.270732838 | loss | 0.825015043 | 0.554282206 | SHANK1 | 37 | 19 |
| cg19214300 | 7.84E−10 | 3.33E−04 | −0.270702835 | 0.270702835 | loss | 0.588899412 | 0.318196576 | MT1IP; MT1IP; MT1IP | 37 | 16 |
| cg10571901 | 7.13E−10 | 3.03E−04 | −0.270700095 | 0.270700095 | loss | 0.842546119 | 0.571846024 | | 37 | 2 |
| cg16430538 | 7.13E−10 | 3.03E−04 | −0.270651007 | 0.270651007 | loss | 0.839373119 | 0.568722112 | ZNF518B | 37 | 4 |
| cg16810031 | 1.43E−08 | 6.06E−03 | −0.270636924 | 0.270636924 | loss | 0.761283647 | 0.490646724 | ZPBP2; ZPBP2 | 37 | 17 |
| cg27046335 | 3.97E−08 | 1.69E−02 | −0.270618061 | 0.270618061 | loss | 0.609772955 | 0.339154894 | PRLR | 37 | 5 |
| cg06399427 | 7.13E−10 | 3.03E−04 | −0.270584897 | 0.270584897 | loss | 0.961351026 | 0.690766129 | | 37 | 2 |
| cg18795569 | 7.13E−10 | 3.03E−04 | −0.270579964 | 0.270579964 | loss | 0.628146275 | 0.357566312 | C19orf77; C19orf77 | 37 | 19 |
| cg14654731 | 7.13E−10 | 3.03E−04 | −0.270543836 | 0.270543836 | loss | 0.607450994 | 0.336907159 | TMEM155; PP12613 | 37 | 4 |
| cg04662609 | 7.13E−10 | 3.03E−04 | −0.270536444 | 0.270536444 | loss | 0.718790221 | 0.448253776 | | 37 | 5 |
| cg00847453 | 7.13E−10 | 3.03E−04 | −0.270474997 | 0.270474997 | loss | 0.731656774 | 0.461181776 | DDO; DOO | 37 | 6 |
| cg13148669 | 7.13E−10 | 3.03E−04 | −0.27045957 | 0.27045957 | loss | 0.801444175 | 0.530984606 | | 37 | 1 |
| cg13986670 | 1.99E−09 | 8.43E−04 | −0.270449436 | 0.270449436 | loss | 0.739032572 | 0.468583135 | FBXO2 | 37 | 1 |
| cg10521948 | 2.56E−09 | 1.09E−03 | −0.270438131 | 0.270438131 | loss | 0.546122772 | 0.275684641 | | 37 | X |
| cg06569542 | 7.13E−10 | 3.03E−04 | −0.270395007 | 0.270395007 | loss | 0.83136396 | 0.560968953 | SLIT1; ARKGAP19-SL1T1 | 37 | 10 |
| cg27434086 | 7.13E−10 | 3.03E−04 | −0.270382366 | 0.270382366 | loss | 0.780280677 | 0.509898312 | APBB2; APBB2; APBB2 | 37 | 4 |
| cg08883853 | 1.71E−09 | 7.28E−04 | −0.270338985 | 0.270338985 | loss | 0.638493256 | 0.368154271 | CYP2W1 | 37 | 7 |
| cg26700447 | 9.73E−10 | 3.92E−04 | −0.270327049 | 0.270327049 | loss | 0.739065108 | 0.468738059 | CCKBR | 37 | 11 |
| cg04295144 | 1.42E−09 | 6.01E−04 | −0.270315179 | 0.270315179 | loss | 0.355045832 | 0.084730653 | ICAM5 | 37 | 19 |
| cg14417784 | 2.56E−09 | 1.09E−03 | −0.270263574 | 0.270263574 | loss | 0.408502809 | 0.138339235 | | 37 | 2 |
| cg19234983 | 4.75E−09 | 2.02E−03 | −0.270238921 | 0.270238921 | loss | 0.70204435 | 0.431805429 | | 37 | 1 |
| cg08979515 | 6.35E−09 | 2.70E−03 | −0.270217502 | 0.270217502 | loss | 0.54476166 | 0.274544159 | ABHD10; ABHD10; ABHD10; | 37 | 3 |
| | | | | | | | | ABHD10 | | |
| cg22697108 | 9.23E−10 | 3.92E−04 | −0.270200058 | 0.270200058 | loss | 0.576990464 | 0.306790406 | | 37 | 5 |
| cg03476791 | 7.13E−10 | 3.03E−04 | −0.270195375 | 0.270195375 | loss | 0.587318181 | 0.317122806 | | 37 | 7 |
| cg01882498 | 1.67E−08 | 7.10E−03 | −0.270181271 | 0.270181271 | loss | 0.634496242 | 0.364314971 | LIN54 | 37 | 4 |
| cg17349199 | 7.77E−10 | 3.30E−04 | −0.270124364 | 0.270124364 | loss | 0.66602527 | 0.395900906 | C10orf82; C10orf82 | 37 | 10 |
| cg13947534 | 2.63E−09 | 1.12E−03 | −0.27008484 | 0.27008484 | loss | 0.621660375 | 0.351575535 | C9orf171; C9orf171 | 37 | 9 |
| cg12924095 | 7.13E−10 | 3.03E−04 | −0.270082893 | 0.270082893 | loss | 0.38733244 | 0.117249547 | LOC100652758; G3BP1; G3BP1 | 37 | 5 |
| cg14501281 | 9.34E−10 | 3.97E−04 | −0.270082253 | 0.270082253 | loss | 0.738029577 | 0.467947324 | | 37 | 2 |
| cg14794655 | 7.13E−10 | 3.03E−04 | −0.27006017 | 0.27006017 | loss | 0.706502153 | 0.436441982 | C2orf73 | 37 | 2 |
| cg11510586 | 7.13E−10 | 3.03E−04 | −0.270059632 | 0.270059632 | loss | 0.294338136 | 0.024278504 | | 37 | 9 |
| cg19547370 | 3.88E−09 | 1.65E−03 | −0.270029153 | 0.270029153 | loss | 0.735699047 | 0.465669894 | PCOLCE2 | 37 | 3 |
| cg06988349 | 1.01E−09 | 4.27E−04 | −0.270025454 | 0.270025454 | loss | 0.886533683 | 0.616508229 | ATOH7; ATOH7 | 37 | 10 |
| cg22486834 | 1.19E−09 | 5.07E−04 | −0.270012492 | 0.270012492 | loss | 0.638753192 | 0.3687407 | GSTA4 | 37 | 6 |
| cg15037663 | 7.13E−10 | 3.03E−04 | −0.269975217 | 0.269975217 | loss | 0.673707664 | 0.403732447 | DFNA5; DFNA5; DFNA5; DFNA5; | 37 | 7 |
| | | | | | | | | DFNA5 | | |
| cg20731937 | 7.13E−10 | 3.03E−04 | −0.269950519 | 0.269950519 | loss | 0.33009454 | 0.060144021 | | 37 | 12 |
| cg11246233 | 3.14E−08 | 1.33E−02 | −0.269852705 | 0.269852705 | loss | 0.867453034 | 0.597600329 | | 37 | 5 |
| cg02799090 | 7.13E−10 | 3.03E−04 | −0.269825095 | 0.269825095 | loss | 0.709063077 | 0.439237982 | | 37 | 10 |
| cg20851828 | 8.47E−10 | 3.60E−04 | −0.269824337 | 0.269824337 | loss | 0.768526296 | 0.498701959 | | 37 | 6 |
| cg22743002 | 7.13E−10 | 3.03E−04 | −0.26974974 | 0.26974974 | loss | 0.55761567 | 0.287865929 | GRM4; GRM4 | 37 | 6 |
| cg07275979 | 2.35E−09 | 9.98E−04 | −0.269748974 | 0.269748974 | loss | 0.910200009 | 0.640451035 | TBC1D31; TBC1D31 | 37 | 8 |
| cg02980055 | 7.77E−10 | 3.30E−04 | −0.269702977 | 0.269702977 | loss | 0.802119689 | 0.532416712 | | 37 | 8 |
| cg05764839 | 7.13E−10 | 3.03E−04 | −0.269684838 | 0.269684838 | loss | 0.314523242 | 0.044838404 | PRRT1 | 37 | 6 |
| cg05096637 | 7.13E−10 | 3.03E−04 | −0.269659452 | 0.269659452 | loss | 0.799827364 | 0.530167912 | | 37 | 2 |
| cg24113950 | 7.13E−10 | 3.03E−04 | −0.269623955 | 0.269623955 | loss | 0.483456223 | 0.213832268 | | 37 | 17 |
| cg02948944 | 7.34E−08 | 3.12E−02 | −0.269613737 | 0.269613737 | loss | 0.889852919 | 0.620239182 | | 37 | 17 |
| cg14153919 | 7.13E−10 | 3.03E−04 | −0.269589149 | 0.269589149 | loss | 0.638375808 | 0.368786659 | CRABP1 | 37 | 15 |
| cg26288449 | 7.13E−10 | 3.03E−04 | −0.269586318 | 0.269586318 | loss | 0.625667353 | 0.356081035 | SBK2; SBX2 | 37 | 19 |
| cg05212464 | 7.13E−10 | 3.03E−04 | −0.269577223 | 0.269577223 | loss | 0.374346325 | 0.104769101 | | 37 | 5 |
| cg09755594 | 1.30E−09 | 5.52E−04 | −0.269531131 | 0.269531131 | loss | 0.81553436 | 0.546003229 | WDR63 | 37 | 1 |
| cg18147098 | 7.77E−10 | 3.30E−04 | −0.269530034 | 0.269530034 | loss | 0.741550275 | 0.472120241 | | 37 | 1 |
| cg18530324 | 7.13E−10 | 3.03E−04 | −0.269499654 | 0.269499654 | loss | 0.633943919 | 0.364444265 | CTIF; CTIF | 37 | 18 |
| cg01287088 | 7.13E−10 | 3.03E−04 | −0.269480497 | 0.269480497 | loss | 0.668805085 | 0.399324588 | PFN3 | 37 | 5 |
| cg02850715 | 7.13E−10 | 3.03E−04 | −0.269448114 | 0.269448114 | loss | 0.697402232 | 0.427954118 | ST14 | 37 | 11 |
| cg19463256 | 9.23E−10 | 3.92E−04 | −0.269413014 | 0.269413014 | loss | 0.734662691 | 0.465249676 | CCDC8 | 37 | 19 |
| cg24927974 | 7.13E−10 | 3.03E−04 | −0.269394012 | 0.269394012 | loss | 0.669080183 | 0.399686171 | DPY19L1 | 37 | 7 |
| cg08474748 | 1.32E−08 | 5.59E−03 | −0.26939377 | 0.26939377 | loss | 0.555078158 | 0.285684388 | | 37 | 5 |
| cg23848052 | 7.13E−10 | 3.03E−04 | −0.269343242 | 0.269343242 | loss | 0.571714283 | 0.302371041 | MEGF11 | 37 | 15 |
| cg22383874 | 7.13E−10 | 3.03E−04 | −0.269311069 | 0.269311069 | loss | 0.785519728 | 0.516208659 | CACNA1G; CACNA1G; CACNA1G; | 37 | 17 |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G | | |
| cg23595451 | 1.19E−09 | 5.07E−04 | −0.269291416 | 0.269291416 | loss | 0.666594792 | 0.397303376 | | 37 | 4 |
| cg06462065 | 7.13E−10 | 3.03E−04 | −0.26928475 | 0.26928475 | loss | 0.450191987 | 0.180907236 | | 37 | 19 |
| cg10356759 | 1.22E−08 | 5.16E−03 | −0.269271453 | 0.269271453 | loss | 0.473275536 | 0.204004082 | IQSEC3; IQSEC3; LOC574538 | 37 | 12 |
| cg19530728 | 7.13E−10 | 3.03E−04 | −0.269256768 | 0.269256768 | loss | 0.754765468 | 0.4855087 | BTBD19 | 37 | 1 |
| cg22814764 | 1.01E−09 | 4.27E−04 | −0.269247939 | 0.269247939 | loss | 0.890154462 | 0.620906524 | C3orf56 | 37 | 3 |
| cg22775123 | 7.13E−10 | 3.03E−04 | −0.269242877 | 0.269242877 | loss | 0.519693925 | 0.250451047 | DAAM2; DAAM2 | 37 | 6 |
| cg23484555 | 7.84E−10 | 3.33E−04 | −0.269229751 | 0.269229751 | loss | 0.86604781 | 0.596818059 | | 37 | 15 |
| cg14348540 | 7.13E−10 | 3.03E−04 | −0.269215281 | 0.269215281 | loss | 0.545604628 | 0.276389347 | SLC39A14; SLC39A14; SLC39A14; | 37 | 8 |
| | | | | | | | | SLC39A14 | | |
| cg11041817 | 2.91E−08 | 1.23E−02 | −0.269189439 | 0.269189439 | loss | 0.610336009 | 0.341146571 | HOXB7 | 37 | 17 |
| cg19766591 | 1.83E−09 | 7.75E−04 | −0.269148045 | 0.269148045 | loss | 0.865331845 | 0.5961838 | | 37 | 10 |
| cg16855352 | 5.39E−09 | 2.29E−03 | −0.269113544 | 0.269113544 | loss | 0.946495421 | 0.677381876 | | 37 | 6 |
| cg15745284 | 9.23E−10 | 3.92E−04 | −0.26908534 | 0.26908534 | loss | 0.86999577 | 0.600910429 | TNXB | 37 | 6 |
| cg12884551 | 7.13E−10 | 3.03E−04 | −0.269007705 | 0.269007705 | loss | 0.459863164 | 0.190855459 | | 37 | 5 |
| cg06834313 | 1.32E−08 | 5.59E−03 | −0.268959931 | 0.268959931 | loss | 0.758970172 | 0.490010241 | | 37 | 12 |
| cg23259375 | 7.13E−10 | 3.03E−04 | −0.268944335 | 0.268944335 | loss | 0.924611817 | 0.655667482 | | 37 | 6 |
| cg11132536 | 7.13E−10 | 3.03E−04 | −0.268939122 | 0.268939122 | loss | 0.876391087 | 0.607451965 | VSX2 | 37 | 14 |
| cg21443699 | 1.54E−08 | 6.56E−03 | −0.268925636 | 0.268925636 | loss | 0.839692842 | 0.570767206 | C7orf34 | 37 | 7 |
| cg10694914 | 1.10E−09 | 4.65E−04 | −0.268890178 | 0.268890178 | loss | 0.566025902 | 0.297135724 | POUZAF1 | 37 | 11 |
| cg05816193 | 7.13E−10 | 3.03E−04 | −0.26886238 | 0.26886238 | loss | 0.437497951 | 0.168635571 | HIST1H1A; HIST1H1A | 37 | 6 |
| cg19065106 | 7.13E−10 | 3.03E−04 | −0.268855113 | 0.268855113 | loss | 0.757857866 | 0.489002753 | SP7; SP7 | 37 | 12 |
| cg24612707 | 7.13E−10 | 3.03E−04 | −0.26883589 | 0.26883589 | loss | 0.556800543 | 0.287964653 | HILPDA; HILPDA | 37 | 7 |
| cg05545777 | 4.29E−08 | 1.82E−02 | −0.268788167 | 0.268788167 | loss | 0.469697619 | 0.200909452 | | 37 | 5 |
| cg15536947 | 2.92E−09 | 1.24E−03 | −0.268758916 | 0.268758916 | loss | 0.497523666 | 0.22876475 | PITPNC1; PITPNC1 | 37 | 17 |
| cg10494684 | 7.13E−10 | 3.03E−04 | −0.26875294 | 0.26875294 | loss | 0.368402492 | 0.099649553 | TMEM97 | 37 | 17 |
| cg26004810 | 1.01E−09 | 4.27E−04 | −0.268723316 | 0.268723316 | loss | 0.649935845 | 0.381212529 | FADD | 37 | 11 |
| cg21862126 | 4.21E−09 | 1.79E−03 | −0.268694286 | 0.268694286 | loss | 0.765981851 | 0.497287565 | LOC254099 | 37 | 1 |
| cg26385222 | 7.13E−10 | 3.03E−04 | −0.268683903 | 0.268683903 | loss | 0.490541279 | 0.221857376 | TMEM176B; TMEM176B; | 37 | 7 |
| | | | | | | | | TMEM176B; TMEM176B; | | |
| | | | | | | | | TMEM176A | | |
| cg16276523 | 7.77E−10 | 3.30E−04 | −0.268673621 | 0.268673621 | loss | 0.866950558 | 0.598276947 | PVRL4 | 37 | 1 |
| cg17527422 | 9.23E−10 | 3.92E−04 | −0.268659549 | 0.268659549 | loss | 0.764337408 | 0.495677859 | C12orf68 | 37 | 12 |
| cg25251478 | 8.47E−10 | 3.60E−04 | −0.268592153 | 0.268592153 | loss | 0.7020987 | 0.433506547 | DDR1; DDR1; DDR1 | 37 | 6 |
| cg09334514 | 7.13E−10 | 3.03E−04 | −0.268586531 | 0.268586531 | loss | 0.562993102 | 0.294406571 | LGALS8; LGALS8; LGALS8; LGALS8; | 37 | 1 |
| | | | | | | | | LGALS8-AS1 | | |
| cg04872399 | 7.13E−10 | 3.03E−04 | −0.268537718 | 0.268537718 | loss | 0.8470759 | 0.578538182 | TNNT2; TNNT2; TNNT2; TNNT2; | 37 | 1 |
| | | | | | | | | TNNT2; TNNT2; TNNT2; | | |
| | | | | | | | | TNNT2; TNNT2; TNNT2; TNNT2; | | |
| | | | | | | | | TNNT2; TNNT2; TNNT2 | | |
| cg03330558 | 3.02E−09 | 1.28E−03 | −0.268517055 | 0.268517055 | loss | 0.754572143 | 0.486055088 | LOC283710 | 37 | 15 |
| cg21474062 | 7.13E−10 | 3.03E−04 | −0.268515611 | 0.268515611 | loss | 0.691807511 | 0.4232919 | ZBTB38 | 37 | 3 |
| cg10612096 | 7.13E−10 | 3.03E−04 | −0.268511034 | 0.268511034 | loss | 0.505835675 | 0.237324641 | SPATA3 | 37 | 2 |
| cg16379671 | 1.67E−08 | 7.10E−03 | −0.268472484 | 0.268472484 | loss | 0.848927113 | 0.580454629 | | 37 | 12 |
| cg09373597 | 7.13E−10 | 3.03E−04 | −0.268454255 | 0.268454255 | loss | 0.463860008 | 0.195405753 | C3orf72 | 37 | 3 |
| cg15205559 | 1.68E−09 | 7.12E−04 | −0.268442219 | 0.268442219 | loss | 0.812573266 | 0.544131047 | | 37 | 4 |
| cg19618897 | 7.13E−10 | 3.03E−04 | −0.268376022 | 0.268376022 | loss | 0.707254098 | 0.438878076 | | 37 | 17 |
| cg13981054 | 7.77E−10 | 3.30E−04 | −0.268369911 | 0.268369911 | loss | 0.665331811 | 0.3969619 | | 37 | 6 |
| cg15326579 | 7.13E−10 | 3.03E−04 | −0.26833909 | 0.26833909 | loss | 0.806234855 | 0.537895765 | | 37 | 2 |
| cg19284039 | 8.55E−08 | 3.63E−02 | −0.268309939 | 0.268309939 | loss | 0.553906492 | 0.285596554 | ASCL2 | 37 | 11 |
| cg18500300 | 7.13E−10 | 3.03E−04 | −0.268287425 | 0.268287425 | loss | 0.472969172 | 0.204681747 | NACAD | 37 | 7 |
| cg00974095 | 7.13E−10 | 3.03E−04 | −0.268200747 | 0.268200747 | loss | 0.785794442 | 0.517593694 | HPCAL4; HPCAL4; HPCAL4 | 37 | 1 |
| cg08139206 | 7.13E−10 | 3.03E−04 | −0.268194918 | 0.268194918 | loss | 0.67440023 | 0.406205312 | | 37 | 14 |
| cg23202468 | 1.54E−09 | 6.54E−04 | −0.268176142 | 0.268176142 | loss | 0.716732277 | 0.448556135 | ZBTB47 | 37 | 3 |
| cg19276110 | 7.13E−10 | 3.03E−04 | −0.268131462 | 0.268131462 | loss | 0.691715857 | 0.423584394 | PLA2G5 | 37 | 1 |
| cg22691119 | 4.97E−09 | 2.11E−03 | −0.268092548 | 0.268092548 | loss | 0.956222377 | 0.688129829 | | 37 | 12 |
| cg07070113 | 4.31E−09 | 1.83E−03 | −0.268090634 | 0.268090634 | loss | 0.754060315 | 0.485969681 | LINC00482 | 37 | 17 |
| cg11672277 | 7.13E−10 | 3.03E−04 | −0.268075223 | 0.268075223 | loss | 0.707995306 | 0.439920082 | MAPRE2 | 37 | 18 |
| cg27176129 | 8.47E−10 | 3.60E−04 | −0.268038915 | 0.268038915 | loss | 0.831816515 | 0.5637776 | RBPJL; RBPJL; MATN4; MATN4; | 37 | 20 |
| | | | | | | | | RBPJL; MATN4; MATN4; | | |
| | | | | | | | | MATN4 | | |
| cg05504117 | 7.84E−10 | 3.33E−04 | −0.268034176 | 0.268034176 | loss | 0.419364348 | 0.151330172 | | 37 | 17 |
| cg00055529 | 8.47E−10 | 3.60E−04 | −0.268026792 | 0.268026792 | loss | 0.418544432 | 0.15051764 | FBXO47; FBXO47 | 37 | 17 |
| cg09100271 | 7.13E−10 | 3.03E−04 | −0.26798885 | 0.26798885 | loss | 0.498604638 | 0.230615788 | | 37 | 8 |
| cg20408181 | 5.41E−08 | 2.30E−02 | −0.267947607 | 0.267947607 | loss | 0.764576583 | 0.496628976 | | 37 | 2 |
| cg07830534 | 1.99E−09 | 8.43E−04 | −0.267937984 | 0.267937984 | loss | 0.839823713 | 0.571885729 | KRT28 | 37 | 17 |
| cg01773685 | 7.13E−10 | 3.03E−04 | −0.267890783 | 0.267890783 | loss | 0.755448342 | 0.487557559 | KLK15; KLK15; KLK15; KLK15 | 37 | 19 |
| cg26383138 | 1.16E−07 | 4.91E−02 | −0.267878963 | 0.267878963 | loss | 0.599183698 | 0.331304735 | MYT1L | 37 | 2 |
| cg15925365 | 7.77E−10 | 3.30E−04 | −0.267854866 | 0.267854866 | loss | 0.635903766 | 0.3680489 | GSTA4 | 37 | 6 |
| cg10196558 | 7.13E−10 | 3.03E−04 | −0.267843145 | 0.267843145 | loss | 0.4063371 | 0.138493955 | | 37 | 1 |
| cg04685442 | 4.64E−08 | 1.97E−02 | −0.267804319 | 0.267804319 | loss | 0.646257377 | 0.378453059 | | 37 | 8 |
| cg09476130 | 1.10E−09 | 4.65E−04 | −0.267796606 | 0.267796606 | loss | 0.604923079 | 0.337126474 | CCDC19; CCDC19 | 37 | 1 |
| cg10321236 | 7.13E−10 | 3.03E−04 | −0.267754102 | 0.267754102 | loss | 0.589251055 | 0.321496953 | MYO7A; MYO7A; MYO7A | 37 | 11 |
| cg16723488 | 9.23E−10 | 3.92E−04 | −0.267723557 | 0.267723557 | loss | 0.366422215 | 0.098698658 | APOB; APOB | 37 | 2 |
| cg00582337 | 7.13E−10 | 3.03E−04 | −0.267688935 | 0.267688935 | loss | 0.931906053 | 0.664217118 | KAT2A; HSPB9 | 37 | 17 |
| cg12065616 | 7.77E−10 | 3.30E−04 | −0.267645858 | 0.267645858 | loss | 0.507230123 | 0.239584265 | MIR5700 | 37 | 12 |
| cg03313542 | 9.23E−10 | 3.92E−04 | −0.267638942 | 0.267638942 | loss | 0.452137838 | 0.184498895 | TMEM209 | 37 | 7 |
| cg25433316 | 7.13E−10 | 3.03E−04 | −0.267572805 | 0.267572805 | loss | 0.369903902 | 0.102331096 | CYS1 | 37 | 2 |
| cg16135695 | 5.39E−09 | 2.29E−03 | −0.267541949 | 0.267541949 | loss | 0.952812749 | 0.6852708 | | 37 | 10 |
| cg08363339 | 8.47E−10 | 3.60E−04 | −0.267473258 | 0.267473258 | loss | 0.833409323 | 0.565936065 | SLMO2; SLMO2; SLMO2- | 37 | 20 |
| | | | | | | | | ATP5E; SLMO2-ATP5E | | |
| cg10612274 | 7.77E−10 | 3.30E−04 | −0.267427478 | 0.267417478 | loss | 0.448586968 | 0.18115949 | MACROD1 | 37 | 11 |
| cg06919203 | 7.84E−10 | 3.33E−04 | −0.26737026 | 0.26737026 | loss | 0.70092416 | 0.4335539 | GFRA4; GFRA4; GFRA4; GFRA4 | 37 | 20 |
| cg04130572 | 7.13E−10 | 3.03E−04 | −0.267367137 | 0.267367137 | loss | 0.683823249 | 0.416456112 | JPH2; JPH2 | 37 | 20 |
| cg25673584 | 8.47E−10 | 3.60E−04 | −0.267334325 | 0.267334325 | loss | 0.694141102 | 0.426806776 | NANOS2 | 37 | 19 |
| cg00363082 | 7.13E−10 | 3.03E−04 | −0.257306819 | 0.267306819 | loss | 0.553117477 | 0.285810659 | | 37 | 5 |
| cg17866778 | 7.13E−10 | 3.03E−04 | −0.267273676 | 0.267273676 | loss | 0.352328426 | 0.085054751 | | 37 | 6 |
| cg20850873 | 1.96E−08 | 8.33E−03 | −0.267265816 | 0.267265816 | loss | 0.914109245 | 0.646843429 | FANCD2OS; FANCD2OS; FANCD2OS; | 37 | 3 |
| | | | | | | | | FANCD2OS | | |
| cg18316498 | 7.77E−10 | 3.30E−04 | −0.267235933 | 0.267235933 | loss | 0.462423421 | 0.195187488 | POU2AF1; POU2AF1 | 37 | 11 |
| cg11166287 | 7.77E−10 | 3.30E−04 | −0.267186226 | 0.267186226 | loss | 0.612728091 | 0.345541865 | | 37 | 14 |
| cg25598308 | 7.77E−10 | 3.30E−04 | −0.267128392 | 0.267128392 | loss | 0.728807345 | 0.461678953 | GRIK4; GRIK4; GRIK4 | 37 | 11 |
| cg17921412 | 7.13E−10 | 3.03E−04 | −0.267111185 | 0.267111185 | loss | 0.524633221 | 0.257522035 | | 37 | 5 |
| cg16037711 | 7.13E−10 | 3.03E−04 | −0.26709591 | 0.26709591 | loss | 0.485294151 | 0.218198241 | LOC100131060; LOC100131060 | 37 | 1 |
| cg08976101 | 7.13E−10 | 3.03E−04 | −0.267037099 | 0.267037099 | loss | 0.550343764 | 0.283306665 | ALX4 | 37 | 11 |
| cg02329835 | 7.13E−10 | 3.03E−04 | −0.267011118 | 0.267011118 | loss | 0.551890142 | 0.284879024 | | 37 | 9 |
| cg15070513 | 8.47E−10 | 3.60E−04 | −0.266997232 | 0.266997232 | loss | 0.605783896 | 0.338786665 | PPP1R1B | 37 | 17 |
| cg14789818 | 9.23E−10 | 3.92E−04 | −0.266986542 | 0.266986542 | loss | 0.333162943 | 0.066176401 | | 37 | 1 |
| cg00807235 | 7.13E−10 | 3.03E−04 | −0.266815385 | 0.266815385 | loss | 0.921868285 | 0.6550529 | | 37 | 16 |
| cg02276070 | 7.13E−10 | 3.03E−04 | −0.266791266 | 0.266791266 | loss | 0.837817766 | 0.5710265 | ARSI | 37 | 5 |
| cg22992730 | 6.35E−09 | 2.70E−03 | −0.266785662 | 0.266785662 | loss | 0.514183962 | 0.247398301 | | 37 | 19 |
| cg11660018 | 7.13E−10 | 3.03E−04 | −0.266770806 | 0.266770806 | loss | 0.585126406 | 0.3133556 | PRSS23 | 37 | 11 |
| cg09077319 | 9.23E−10 | 3.92E−04 | −0.266735935 | 0.266735935 | loss | 0.617583764 | 0.350847829 | | 37 | 11 |
| cg09467436 | 1.19E−09 | 5.07E−04 | −0.266712611 | 0.266712611 | loss | 0.689735758 | 0.423023147 | | 37 | 2 |
| cg11287647 | 1.10E−09 | 4.65E−04 | 0.266699855 | 0.266699855 | loss | 0.628828749 | 0.362128894 | ARL4A; ARL4A | 37 | 7 |
| cg14382976 | 1.54E−09 | 6.54E−04 | −0.266646278 | 0.266646278 | loss | 0.839233149 | 0.572586871 | ASZ1; ASZ1; AS21; ASZ1 | 37 | 7 |
| cg20772512 | 7.84E−10 | 3.33E−04 | −0.266637156 | 0.266637156 | loss | 0.380814392 | 0.114177236 | DNAI1 | 37 | 1 |
| cg01209984 | 7.13E−10 | 3.03E−04 | −0.266635187 | 0.266635187 | loss | 0.68489917 | 0.418263982 | | 37 | 21 |
| cg02623200 | 1.01E−09 | 4.27E−04 | −0.266615008 | 0.266615008 | loss | 0.616862408 | 0.3502474 | SPAG17; SPAG17 | 37 | 1 |
| cg08774009 | 7.13E−10 | 3.03E−04 | −0.266610076 | 0.266610076 | loss | 0.447539464 | 0.180929388 | | 37 | 8 |
| cg02438164 | 1.19E−09 | 5.07E−04 | −0.266593272 | 0.266593272 | loss | 0.529097866 | 0.262504594 | | 37 | 16 |
| cg09141303 | 2.78E−09 | 1.18E−03 | −0.266551371 | 0.266551371 | loss | 0.712980094 | 0.446428724 | SLC9B2; SLC9B2 | 37 | 4 |
| cg24996985 | 7.77E−10 | 3.30E−04 | −0.266523823 | 0.266523823 | loss | 0.552582511 | 0.286058688 | | 37 | 2 |
| cg00435606 | 7.13E−10 | 3.03E−04 | −0.266523105 | 0.266523105 | loss | 0.786877428 | 0.520354324 | CENPV | 37 | 17 |
| cg06239923 | 7.84E−10 | 3.33E−04 | −0.266507464 | 0.266507464 | loss | 0.938528458 | 0.672020994 | MIR5095 | 37 | 20 |
| cg26621607 | 1.30E−09 | 5.52E−04 | −0.266482781 | 0.266482781 | loss | 0.508941775 | 0.242458994 | LRCOL1 | 37 | 12 |
| cg13846270 | 7.77E−10 | 3.30E−04 | −0.266477099 | 0.265477099 | loss | 0.449220642 | 0.182743543 | DLEU7; DLEU7; DLEU7- | 37 | 13 |
| | | | | | | | | AS1 | | |
| cg06974662 | 8.47E−10 | 3.60E−04 | −0.266466286 | 0.266466286 | loss | 0.504779509 | 0.238313224 | | 37 | 1 |
| cg26107597 | 7.13E−10 | 3.03E−04 | −0.266422855 | 0.266422855 | loss | 0.464263496 | 0.197840641 | COCH; COCH | 37 | 14 |
| cg09618778 | 7.13E−10 | 3.03E−04 | −0.266417292 | 0.266417292 | loss | 0.5102797 | 0.243862408 | RHOD | 37 | 11 |
| cg13891539 | 8.14E−09 | 3.46E−03 | −0.266409632 | 0.266409632 | loss | 0.723779489 | 0.457369856 | | 37 | 12 |
| cg00583535 | 7.77E−10 | 3.30E−04 | −0.266406529 | 0.266406529 | loss | 0.423524083 | 0.157117554 | IGFN1 | 37 | 1 |
| cg05506600 | 1.68E−09 | 7.12E−04 | −0.266390313 | 0.266390313 | loss | 0.48181193 | 0.215421618 | | 37 | 15 |
| cg21708738 | 1.03E−08 | 4.39E−03 | −0.266390043 | 0.266390043 | loss | 0.760889808 | 0.494499765 | C2orf91 | 37 | 2 |
| cg02830202 | 7.77E−10 | 3.30E−04 | −0.266351592 | 0.266351592 | loss | 0.769207462 | 0.502855871 | | 37 | 16 |
| cg08352755 | 7.13E−10 | 3.03E−04 | −0.266344419 | 0.256344419 | loss | 0.464487308 | 0.198142888 | | 37 | 5 |
| cg15056966 | 7.13E−10 | 3.03E−04 | −0.266333139 | 0.266333139 | loss | 0.528497457 | 0.262164318 | | 37 | 3 |
| cg24720336 | 7.13E−10 | 3.03E−04 | −0.266331262 | 0.266331262 | loss | 0.389299657 | 0.122968394 | PIP5K1A; PIP5K1A; PIP5K1A; | 37 | 1 |
| | | | | | | | | PIP5K1A | | |
| cg03365937 | 7.13E−10 | 3.03E−04 | −0.266249034 | 0.266249034 | loss | 0.839453534 | 0.5732045 | CHST1 | 37 | 11 |
| cg16057826 | 7.13E−10 | 3.03E−04 | −0.266219883 | 0.266219883 | loss | 0.536895689 | 0.270675806 | CLIC6 | 37 | 21 |
| cg23587980 | 7.13E−10 | 3.03E−04 | −0.266175089 | 0.266175089 | loss | 0.569098977 | 0.302923888 | KRT86 | 37 | 12 |
| cg03242265 | 7.77E−10 | 3.30E−04 | −0.26614104 | 0.26614104 | loss | 0.500726634 | 0.234585594 | NBPF8; NBPF8; NBPF8 | 37 | 1 |
| cg06532268 | 8.80E−09 | 3.74E−03 | −0.266127515 | 0.266127515 | loss | 0.686731957 | 0.420604441 | HIST1H2APS1 | 37 | 6 |
| cg19987219 | 7.77E−10 | 3.30E−04 | −0.26607125 | 0.26607125 | loss | 0.834030785 | 0.567959535 | KNCN; KNCN; MKNX1-A51 | 37 | 1 |
| cg17121140 | 7.13E−10 | 3.03E−04 | −0.266060897 | 0.266060897 | loss | 0.321964632 | 0.055903735 | | 37 | 13 |
| cg26055950 | 7.13E−10 | 3.03E−04 | −0.266059371 | 0.266059371 | loss | 0.359502806 | 0.093443435 | TP73; TP73; TP73; TP73; TP73; | 37 | 1 |
| | | | | | | | | TP73 | | |
| cg03555157 | 2.16E−09 | 9.17E−04 | −0.265993517 | 0.265993517 | loss | 0.61555787 | 0.349564353 | LIMS2; LIMS2 | 37 | 2 |
| cg14329783 | 3.40E−08 | 1.44E−02 | −0.265973377 | 0.265973377 | loss | 0.577029989 | 0.311056612 | | 37 | 7 |
| cg03400335 | 7.13E−10 | 3.03E−04 | −0.26596524 | 0.26596524 | loss | 0.781700928 | 0.515735688 | ILDR1; ILDR1; ILDR1 | 37 | 3 |
| cg09359103 | 9.54E−09 | 4.05E−03 | −0.265952255 | 0.265952255 | loss | 0.556169702 | 0.290217447 | KCNN3; KCNN3 | 37 | 1 |
| cg18041277 | 1.30E−09 | 5.52E−04 | −0.265828174 | 0.265828174 | loss | 0.774652309 | 0.508824135 | | 37 | 8 |
| cg17518825 | 3.14E−08 | 1.33E−02 | −0.265822068 | 0.265822068 | loss | 0.652053921 | 0.386231853 | EPAS1 | 37 | 2 |
| cg07508649 | 7.13E−10 | 3.03E−04 | −0.265815248 | 0.265815248 | loss | 0.523813889 | 0.257998641 | C2; C2 | 37 | 6 |
| cg13623268 | 6.66E−08 | 2.83E−02 | −0.265804722 | 0.265804722 | loss | 0.72217549 | 0.456370769 | PCSK5 | 37 | 9 |
| cg10671830 | 1.19E−09 | 5.07E−04 | 0.265791792 | 0.265791792 | gain | 0.234938355 | 0.500730147 | SORCS2 | 37 | 4 |
| cg06889571 | 1.35E−08 | 5.71E−03 | −0.265781527 | 0.265781527 | loss | 0.907459683 | 0.641678156 | ADARB2 | 37 | 10 |
| cg10123421 | 1.42E−09 | 6.01E−04 | 0.26575448 | 0.26575448 | gain | 0.305665385 | 0.571419865 | ZNF833P | 37 | 19 |
| cg11068289 | 7.13E−10 | 3.03E−04 | −0.265747582 | 0.265747582 | loss | 0.795592406 | 0.529944824 | | 37 | 17 |
| cg25821249 | 7.13E−10 | 3.03E−04 | −0.26574206 | 0.26574206 | loss | 0.812909272 | 0.547167212 | | 37 | 17 |
| cg08601628 | 1.54E−09 | 6.54E−04 | −0.265699134 | 0.265699134 | loss | 0.820276323 | 0.554577188 | C8orf74 | 37 | 8 |
| cg17352045 | 1.01E−09 | 4.27E−04 | −0.265662348 | 0.265662348 | loss | 0.589189519 | 0.323527171 | | 37 | 6 |
| cg18753928 | 9.54E−09 | 4.05E−03 | −0.265658013 | 0.265658013 | loss | 0.419797926 | 0.154139914 | SPICE1 | 37 | 3 |
| cg08373187 | 7.13E−10 | 3.03E−04 | −0.265626657 | 0.265626657 | loss | 0.543648298 | 0.278021641 | CXCL14 | 37 | 5 |
| cg16111190 | 1.19E−09 | 5.07E−04 | −0.265619921 | 0.265619921 | loss | 0.754582815 | 0.488962894 | DDR1; DDR1; DDR1; DDR1; | 37 | 6 |
| | | | | | | | | DDR1; DDR1 | | |
| cg16500916 | 7.13E−10 | 3.03E−04 | −0.265611023 | 0.265611023 | loss | 0.839360711 | 0.573749688 | | 37 | 16 |
| cg15089314 | 7.13E−10 | 3.03E−04 | −0.265560781 | 0.265560781 | loss | 0.676948528 | 0.411387747 | BHMT2; DMGDH; BHMT2; | 37 | 5 |
| | | | | | | | | DMGDH; DMGDH | | |
| cg22167400 | 7.13E−10 | 3.03E−04 | −0.265559566 | 0.265559566 | loss | 0.561432725 | 0.295873159 | KRT18P55 | 37 | 17 |
| cg08245096 | 7.13E−10 | 3.03E−04 | −0.265559095 | 0.665559095 | loss | 0.659274989 | 0.393715894 | | 37 | 7 |
| cg08685733 | 1.68E−09 | 7.12E−04 | −0.2655377 | 0.2655377 | loss | 0.424940853 | 0.159403153 | | 37 | 17 |
| cg06724402 | 6.35E−09 | 2.70E−03 | −0.265492344 | 0.265492344 | loss | 0.775135768 | 0.509643424 | ANGPTL5 | 37 | 11 |
| cg15209566 | 7.77E−10 | 3.30E−04 | 0.265448171 | 0.265448171 | gain | 0.064197847 | 0.329646018 | ZNF833P | 37 | 19 |
| cg06084073 | 7.13E−10 | 3.03E−04 | −0.265369154 | 0.265369154 | loss | 0.80112496 | 0.535755806 | C15orf56; PAK6; PAK6; PAK6; | 37 | 15 |
| | | | | | | | | PAK6; PAK6 | | |
| cg12717963 | 7.84E−10 | 3.33E−04 | −0.265297543 | 0.265297543 | loss | 0.911956231 | 0.646658688 | TRIM7; TRIM7; TRIM7; TRIM7; | 37 | 5 |
| | | | | | | | | TRIM7 | | |
| cg01431140 | 7.13E−10 | 3.03E−04 | −0.265292927 | 0.265292927 | loss | 0.942089174 | 0.676796247 | | 37 | 17 |
| cg23053961 | 2.56E−09 | 1.09E−03 | −0.265286158 | 0.265286158 | loss | 0.652527281 | 0.387241124 | GLIS3 | 37 | 9 |
| cg04391540 | 1.73E−09 | 7.34E−04 | −0.265261816 | 0.265261816 | loss | 0.500798728 | 0.235536913 | ZNF541 | 37 | 19 |
| cg07207043 | 7.13E−10 | 3.03E−04 | −0.265164374 | 0.265164374 | loss | 0.653772004 | 0.388607629 | | 37 | 6 |
| cg18938392 | 2.30E−08 | 9.75E−03 | −0.265098195 | 0.265098195 | loss | 0.757870572 | 0.492772376 | | 37 | 1 |
| cg17436656 | 7.13E−10 | 3.03E−04 | −0.265042387 | 0.265042387 | loss | 0.65096357 | 0.385921182 | RARG; RARG; RARG | 37 | 12 |
| cg05645982 | 9.45E−10 | 4.01E−04 | −0.26499208 | 0.26499208 | loss | 0.582386375 | 0.317394294 | FAM83A; FAM83A | 37 | 8 |
| cg04299200 | 2.03E−09 | 8.64E−04 | −0.264987648 | 0.264987648 | loss | 0.769497337 | 0.504509688 | | 37 | 5 |
| cg24068229 | 7.13E−10 | 3.03E−04 | −0.26494425 | 0.26494425 | loss | 0.361283368 | 0.096339118 | FBXO27; FBXO27 | 37 | 19 |
| cg04573398 | 9.23E−10 | 3.92E−04 | −0.264915806 | 0.264915806 | loss | 0.848526047 | 0.583610241 | CCDC81; CCDC81 | 37 | 11 |
| cg16791134 | 1.99E−09 | 8.43E−04 | −0.264913525 | 0.264913525 | loss | 0.706361525 | 0.441448 | SYN2; TIMP4; TIMP4; SYN2 | 37 | 3 |
| cg13191951 | 3.14E−08 | 1.33E−02 | −0.264910446 | 0.264910446 | loss | 0.83417254 | 0.569262094 | THSD4 | 37 | 15 |
| cg15235893 | 7.13E−10 | 3.03E−04 | −0.26489749 | 0.26489749 | loss | 0.760589043 | 0.495691553 | ARL4C; ARL4C | 37 | 2 |
| cg01227744 | 2.91E−08 | 1.23E−02 | −0.264878797 | 0.264878797 | loss | 0.748920132 | 0.484041335 | | 37 | 2 |
| cg12582028 | 7.13E−10 | 3.03E−04 | −0.264872876 | 0.264872876 | loss | 0.703398858 | 0.438525982 | OAZ3; MRPL9 | 37 | 1 |
| cg03162823 | 7.13E−10 | 3.03E−04 | −0.264859543 | 0.264859543 | loss | 0.60949626 | 0.344636718 | USP44; USP44; USP44 | 37 | 12 |
| cg26280727 | 6.80E−08 | 2.89E−02 | −0.264356229 | 0.264856229 | loss | 0.759486111 | 0.494629882 | LMTK3 | 37 | 19 |
| cg20075675 | 7.77E−10 | 3.30E−04 | −0.264799132 | 0.264799132 | loss | 0.647144626 | 0.382345494 | NUAK1 | 37 | 12 |
| cg18323095 | 7.13E−10 | 3.03E−04 | −0.264716329 | 0.264716329 | loss | 0.929303917 | 0.664587588 | | 37 | 15 |
| cg12301695 | 1.22E−08 | 5.16E−03 | −0.264694271 | 0.264694271 | loss | 0.577167583 | 0.312473312 | SLC38A11; SLC38A11; SLC38A11; | 37 | 2 |
| | | | | | | | | SLC38A11 | | |
| cg17321385 | 7.77E−10 | 3.30E−04 | −0.264660938 | 0.264660938 | loss | 0.537150091 | 0.272489153 | | 37 | 2 |
| cg23397023 | 7.13E−10 | 3.03E−04 | −0.264644494 | 0.264644494 | loss | 0.595538794 | 0.3308943 | | 37 | 7 |
| cg05407338 | 8.47E−10 | 3.60E−04 | −0.264630332 | 0.264630332 | loss | 0.443412738 | 0.178782406 | ARHGEF15; ARHGEF15 | 37 | 17 |
| cg26934993 | 7.13E−10 | 3.03E−04 | −0.264598862 | 0.264598862 | loss | 0.906304226 | 0.641705365 | KAT2A; HSP89 | 37 | 17 |
| cg21211253 | 7.13E−10 | 3.03E−04 | −0.264582092 | 0.264582092 | loss | 0.871955715 | 0.607373624 | | 37 | 5 |
| cg04319873 | 7.13E−10 | 3.03E−04 | −0.264573954 | 0.264573954 | loss | 0.298483777 | 0.033909823 | | 37 | 2 |
| cg14466942 | 7.13E−10 | 3.03E−04 | −0.26456419 | 0.26456419 | loss | 0.567932108 | 0.303367918 | RBM24; RBM24; RBM24 | 37 | 6 |
| cg13595308 | 7.13E−10 | 3.03E−04 | −0.264548422 | 0.264548422 | loss | 0.784250881 | 0.519702459 | | 37 | 9 |
| cg02608963 | 7.13E−10 | 3.03E−04 | −0.264534462 | 0.264534462 | loss | 0.721650021 | 0.457115559 | | 37 | 16 |
| cg09031776 | 1.83E−09 | 7.75E−04 | −0.264508902 | 0.264508902 | loss | 0.51708206 | 0.252573159 | | 37 | 10 |
| cg17052552 | 7.13E−10 | 3.03E−04 | −0.264484531 | 0.264484531 | loss | 0.946472202 | 0.681987671 | ANK1; ANK1; ANK1; ANK1; | 37 | 8 |
| | | | | | | | | ANK1 | | |
| cg23629935 | 7.13E−10 | 3.03E−04 | −0.264482472 | 0.264482472 | loss | 0.762764113 | 0.498281641 | | 37 | 14 |
| cg12244617 | 7.13E−10 | 3.03E−04 | −0.264468251 | 0.264468251 | loss | 0.415641274 | 0.151173022 | PLXNB2 | 37 | 22 |
| cg19916129 | 7.84E−10 | 3.33E−04 | −0.26440799 | 0.26440799 | loss | 0.610005185 | 0.345597194 | TUBGCP3; TUBGCP3; TUBGCP3; | 37 | 13 |
| | | | | | | | | TUBGCP3 | | |
| cg24735235 | 2.35E−09 | 9.98E−04 | −0.264396197 | 0.264396197 | loss | 0.458498608 | 0.194102411 | FLJ37453 | 37 | 1 |
| cg15442037 | 7.13E−10 | 3.03E−04 | −0.264373392 | 0.264373392 | loss | 0.487270609 | 0.222897218 | | 37 | 11 |
| cg09384937 | 7.13E−10 | 3.03E−04 | −0.264331391 | 0.264331391 | loss | 0.796433085 | 0.532101694 | WNT10B | 37 | 12 |
| cg00965168 | 7.13E−10 | 3.03E−04 | −0.264293783 | 0.264293783 | loss | 0.69166663 | 0.427372847 | | 37 | 1 |
| cg17878899 | 2.78E−09 | 1.18E−03 | −0.264291397 | 0.264291397 | loss | 0.685799485 | 0.421508088 | KIAA1210; KIAA1210 | 37 | X |
| cg22904173 | 7.13E−10 | 3.03E−04 | −0.264284975 | 0.264284975 | loss | 0.646963328 | 0.382678353 | | 37 | 11 |
| cg00202454 | 2.16E−09 | 9.17E−04 | −0.264283981 | 0.264283981 | loss | 0.924561523 | 0.660277541 | PLA2G4D | 37 | 15 |
| cg03759229 | 1.10E−09 | 4.65E−04 | −0.264280637 | 0.264280637 | loss | 0.51562336 | 0.251342724 | | 37 | 6 |
| cg15772157 | 7.13E−10 | 3.03E−04 | −0.26425341 | 0.26425341 | loss | 0.408538326 | 0.144284916 | | 37 | 10 |
| cg16602316 | 3.04E−09 | 1.29E−03 | −0.264247067 | 0.264247067 | loss | 0.320877544 | 0.056630477 | | 37 | 14 |
| cg04598224 | 7.77E−10 | 3.30E−04 | 0.264246083 | 0.264246083 | gain | 0.122124835 | 0.386370918 | ZNF833P | 37 | 19 |
| cg08107402 | 7.13E−10 | 3.03E−04 | −0.264243493 | 0.264243493 | loss | 0.61147554 | 0.347232047 | SCGB1B2P | 37 | 19 |
| cg24702147 | 7.13E−10 | 3.03E−04 | 0.264241932 | 0.264241932 | loss | 0.949576085 | 0.685334153 | MIR141; MIR200C | 37 | 12 |
| cg00509207 | 7.13E−10 | 3.03E−04 | −0.264177587 | 0.264177587 | loss | 0.63202224 | 0.367844653 | TSPAN11 | 37 | 12 |
| cg18493761 | 1.43E−08 | 6.06E−03 | 0.264163727 | 0.264163727 | loss | 0.695227874 | 0.431064147 | | 37 | 11 |
| cg12564567 | 6.35E−09 | 2.70E−03 | −0.264146827 | 0.264146827 | loss | 0.399205596 | 0.135058769 | | 37 | 11 |
| cg20307496 | 7.13E−10 | 3.03E−04 | −0.264145319 | 0.264145319 | loss | 0.841696519 | 0.5775512 | CDC42EP1 | 37 | 22 |
| cg15244786 | 7.13E−10 | 3.03E−04 | −0.264142173 | 0.264142173 | loss | 0.554765638 | 0.290623465 | HOXC4; HOXC4 | 37 | 12 |
| cg10422709 | 7.13E−10 | 3.03E−04 | −0.264141202 | 0.264141202 | loss | 0.672671749 | 0.408530547 | | 37 | 15 |
| cg16773028 | 7.13E−10 | 3.03E−04 | −0.264105046 | 0.264105046 | loss | 0.857458134 | 0.593353088 | KCNA2; KCNA2 | 37 | 1 |
| cg06889535 | 3.69E−09 | 1.57E−03 | −0.264102627 | 0.264102627 | loss | 0.529021998 | 0.264919371 | KIRREL3; KIRREL3 | 37 | 11 |
| cg03463990 | 2.90E−15 | 1.23E−09 | −0.264038185 | 0.264038185 | loss | 0.855128215 | 0.591090029 | CCDC33 | 37 | 15 |
| cg25162921 | 7.13E−10 | 3.03E−04 | −0.264010102 | 0.264010102 | loss | 0.558877102 | 0.294867 | MTAP | 37 | 9 |
| cg16149919 | 1.83E−09 | 7.75E−04 | −0.263981424 | 0.263981424 | loss | 0.7879117 | 0.523930276 | PGPEP1L; PGPEP1L; PGPEP1L | 37 | 15 |
| cg23591302 | 1.22E−08 | 5.16E−03 | −0.263962934 | 0.263962934 | loss | 0.96172694 | 0.697764006 | PRICKLE1; PRICKLE1; PRICKLE1; | 37 | 12 |
| | | | | | | | | PRICKLE1 | | |
| cg08958251 | 1.19E−09 | 5.07E−04 | −0.263953071 | 0.263953071 | loss | 0.789336147 | 0.525383076 | | 37 | 11 |
| cg02136452 | 7.13E−10 | 3.03E−04 | −0.263945043 | 0.263945043 | loss | 0.333965087 | 0.070020044 | ZNF423 | 37 | 16 |
| cg27315077 | 7.13E−10 | 3.03E−04 | −0.263792141 | 0.263792141 | loss | 0.586826406 | 0.323034265 | | 37 | 21 |
| cg01310397 | 7.34E−08 | 3.12E−02 | −0.263778195 | 0.263778195 | loss | 0.79730783 | 0.533529635 | MUC4; MUC4; MUC4 | 37 | 3 |
| cg23507802 | 6.89E−09 | 2.93E−03 | −0.263735102 | 0.263735102 | loss | 0.804989208 | 0.541254106 | WNT7A | 37 | 3 |
| cg16581017 | 1.10E−09 | 4.65E−04 | −0.263723845 | 0.263723845 | loss | 0.713327098 | 0.449603253 | MYL10 | 37 | 7 |
| cg11507770 | 1.54E−09 | 6.54E−04 | −0.263712638 | 0.263712638 | loss | 0.399580002 | 0.135867364 | | 37 | 11 |
| cg19728382 | 7.13E−10 | 3.03E−04 | −0.263660667 | 0.263660667 | loss | 0.478642443 | 0.214981776 | STC2 | 37 | 5 |
| cg07160793 | 8.47E−10 | 3.60E−04 | −0.263650664 | 0.263650664 | loss | 0.689526864 | 0.4258762 | | 37 | 2 |
| cg15489799 | 7.13E−10 | 3.03E−04 | −0.263600633 | 0.263600633 | loss | 0.546355298 | 0.282754665 | | 37 | 1 |
| cg00469015 | 7.13E−10 | 3.03E−04 | −0.263585279 | 0.263585279 | loss | 0.319321526 | 0.055736248 | RPLP1; RPLP1 | 37 | 15 |
| cg25325636 | 7.13E−10 | 3.03E−04 | −0.263529998 | 0.263529998 | loss | 0.680735162 | 0.417205165 | ZNF831 | 37 | 20 |
| cg06895640 | 1.01E−09 | 4.27E−04 | −0.263504457 | 0.263504457 | loss | 0.840139434 | 0.576634976 | SMCO4 | 37 | 11 |
| cg09893504 | 7.13E−10 | 3.03E−04 | −0.263498069 | 0.263498069 | loss | 0.549340951 | 0.285842882 | C2orf50 | 37 | 2 |
| cg01820273 | 1.12E−08 | 4.76E−03 | −0.263472855 | 0.263472855 | loss | 0.643903838 | 0.380430982 | CLPSL1 | 37 | 6 |
| cg05674602 | 7.13E−10 | 3.03E−04 | −0.263430822 | 0.263430822 | loss | 0.368130809 | 0.104699988 | | 37 | 2 |
| cg11367633 | 7.13E−10 | 3.03E−04 | −0.263426084 | 0.263426084 | loss | 0.498174619 | 0.234748535 | CHD6 | 37 | 20 |
| cg22284422 | 1.67E−08 | 7.10E−03 | −0.26339418 | 0.26339418 | loss | 0.897983951 | 0.634589771 | LINC00908 | 37 | 18 |
| cg08896939 | 7.13E−10 | 3.03E−04 | −0.263390208 | 0.263390208 | loss | 0.405707462 | 0.142317255 | RNF135; RNF135; RNF135 | 37 | 17 |
| cg06809326 | 1.68E−09 | 7.12E−04 | −0.26337068 | 0.26337068 | loss | 0.750167857 | 0.486797176 | CCDC144NL; CCDC144NL; | 37 | 17 |
| | | | | | | | | LOC440416 | | |
| cg27282900 | 1.32E−08 | 5.59E−03 | −0.263318975 | 0.263318975 | loss | 0.343252642 | 0.079933666 | | 37 | 6 |
| cg18263397 | 6.35E−09 | 2.70E−03 | −0.263291461 | 0.263291461 | loss | 0.607121291 | 0.343829829 | ZCCHC16; TRPC5 | 37 | X |
| cg12544557 | 7.13E−10 | 3.03E−04 | −0.263281107 | 0.263281107 | loss | 0.567001713 | 0.303720606 | KRT12 | 37 | 17 |
| cg18632631 | 7.13E−10 | 3.03E−04 | −0.263257636 | 0.263257636 | loss | 0.692614172 | 0.429356535 | TNK1; TNK1 | 37 | 17 |
| cg12258454 | 7.13E−10 | 3.03E−04 | −0.263188648 | 0.263188648 | loss | 0.789138289 | 0.525949641 | | 37 | 3 |
| cg07747661 | 7.77E−10 | 3.30E−04 | −0.263165078 | 0.263165078 | loss | 0.888909408 | 0.625744329 | PKNOX2 | 37 | 11 |
| cg01066472 | 5.84E−08 | 2.48E−02 | −0.263152477 | 0.263152477 | loss | 0.384207082 | 0.121054605 | | 37 | 1 |
| cg09606832 | 7.13E−10 | 3.03E−04 | 0.263137134 | 0.263137134 | loss | 0.691694987 | 0.428557853 | TFAP2A; TFAP2A; TFAP2A | 37 | 6 |
| cg06365016 | 7.13E−10 | 3.03E−04 | −0.263133465 | 0.263133465 | loss | 0.750175794 | 0.487042329 | PIWIL1; PIWIL1 | 37 | 12 |
| cg04994481 | 9.23E−10 | 3.92E−04 | −0.263127617 | 0.263127617 | loss | 0.905528023 | 0.642400406 | | 37 | 14 |
| cg25607643 | 7.13E−10 | 3.03E−04 | −0.263110012 | 0.263110012 | loss | 0.644998594 | 0.381888582 | | 37 | 15 |
| cg19380722 | 1.10E−09 | 4.65E−04 | −0.263067557 | 0.263067557 | loss | 0.775745592 | 0.512678035 | PKD2L1; PKD2L1; PKD2L1; | 37 | 10 |
| | | | | | | | | PKD2L1 | | |
| cg17642369 | 1.42E−09 | 6.01E−04 | −0.263050913 | 0.263050913 | loss | 0.355879592 | 0.092828679 | | 37 | 7 |
| cg08334034 | 5.85E−09 | 2.49E−03 | −0.263041664 | 0.263041664 | loss | 0.686458664 | 0.423417 | RGMA; RGMA; RGMA; RGMA; | 37 | 15 |
| | | | | | | | | RGMA; RGMA | | |
| cg05496603 | 5.85E−09 | 2.49E−03 | −0.263029724 | 0.263029724 | loss | 0.800433594 | 0.537403871 | TMEM151B | 37 | 6 |
| cg11968118 | −7.13E−10 | 3.03E−04 | −0.262994508 | 0.262994508 | loss | 0.702924555 | 0.439930047 | | 37 | 17 |
| cg26906998 | 1.30E−09 | 5.52E−04 | −0.262990294 | 0.262990294 | loss | 0.333394682 | 0.070404388 | PLD6 | 37 | 17 |
| cg23021340 | 2.78E−09 | 1.18E−03 | −0.26297992 | 0.26297992 | loss | 0.782173143 | 0.519193224 | METTL24 | 37 | 6 |
| cg25620797 | 1.01E−09 | 4.27E−04 | −0.262964516 | 0.262964516 | loss | 0.864373734 | 0.601409218 | PNPT1 | 37 | 2 |
| cg17179314 | 4.94E−13 | 2.10E−07 | −0.262924848 | 0.262924848 | loss | 0.510046463 | 0.247121615 | CHRNB4; CHRNB4 | 37 | 15 |
| cg01111041 | 7.13E−10 | 3.03E−04 | −0.262922058 | 0.262922058 | loss | 0.467671902 | 0.204749844 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg20003893 | 7.13E−10 | 3.03E−04 | −0.262879506 | 0.262879506 | loss | 0.682926606 | 0.4200471 | | 37 | 8 |
| cg12379307 | 7.13E−10 | 3.03E−04 | −0.262856944 | 0.262856944 | loss | 0.750683432 | 0.487826488 | SMPDL3A; SMPDL3A | 37 | 6 |
| cg26561986 | 7.13E−10 | 3.03E−04 | −0.26284949 | 0.26284949 | loss | 0.857751743 | 0.594902253 | USP44; USP44; USP44 | 37 | 12 |
| cg01700504 | 2.16E−09 | 9.17E−04 | −0.262836507 | 0.262836507 | loss | 0.581334119 | 0.318497612 | GJB6; GJB6; GJB6; GJB6; GJB6 | 37 | 13 |
| cg15520962 | 7.13E−10 | 3.03E−04 | −0.262831409 | 0.262831409 | loss | 0.733763632 | 0.470932224 | TMEM190 | 37 | 19 |
| cg04059762 | 7.13E−10 | 3.03E−04 | −0.262823708 | 0.262823708 | loss | 0.890760008 | 0.6279363 | | 37 | 16 |
| cg17757980 | 9.23E−10 | 3.92E−04 | −0.262801605 | 0.262801605 | loss | 0.778596017 | 0.515794412 | SYT15; SYT15 | 37 | 10 |
| cg16570507 | 7.13E−10 | 3.03E−04 | −0.262801513 | 0.262801513 | loss | 0.470954877 | 0.208153365 | GRID1; MIR346; MIR346 | 37 | 10 |
| cg02445968 | 3.02E−09 | 1.28E−03 | −0.26278735 | 0.26278735 | loss | 0.904104732 | 0.641317382 | ADARB2 | 37 | 10 |
| cg00000029 | 1.10E−09 | 4.65E−04 | −0.262778712 | 0.262778712 | loss | 0.574659636 | 0.311880924 | RBL2 | 37 | 16 |
| cg01984511 | 7.13E−10 | 3.03E−04 | −0.262767136 | 0.262767136 | loss | 0.733353636 | 0.4705865 | DTX1 | 37 | 12 |
| cg14435498 | 7.13E−10 | 3.03E−04 | −0.262756221 | 0.262756221 | loss | 0.432772768 | 0.170016547 | | 37 | 10 |
| cg17987384 | 7.13E−10 | 3.03E−04 | −0.26266311 | 0.26266311 | loss | 0.753571434 | 0.490908324 | KRT18P55; KRT1SP55 | 37 | 17 |
| cg04100190 | 7.13E−10 | 3.03E−04 | −0.262652411 | 0.262652411 | loss | 0.68357887 | 0.420926459 | COL9A2 | 37 | 1 |
| cg15467577 | 8.47E−10 | 3.60E−04 | −0.262642712 | 0.262642712 | loss | 0.5787684 | 0.316125688 | HPN; HPN | 37 | 19 |
| cg25521622 | 7.13E−10 | 3.03E−04 | −0.262616545 | 0.262616545 | loss | 0.887413557 | 0.624797012 | | 37 | 2 |
| cg06760077 | 7.13E−10 | 3.03E−04 | −0.262608758 | 0.262608758 | loss | 0.553474781 | 0.290866024 | | 37 | 2 |
| cg20655369 | 4.97E−09 | 2.11E−03 | −0.262571997 | 0.262571997 | loss | 0.811164185 | 0.548592188 | LINC00925 | 37 | 15 |
| cg02227331 | 7.13E−10 | 3.03E−04 | −0.262516475 | 0.262516475 | loss | 0.803287928 | 0.540771453 | UGT1A6; UGT1A4; UGT1A10; | 37 | 2 |
| | | | | | | | | UGT1A8; UGT1A7; UGT1A5; | | |
| | | | | | | | | UGT1A3; UGT1A9; UGT1A6; | | |
| | | | | | | | | LOC100286922; LOC100286922; | | |
| | | | | | | | | LOC100286922; | | |
| | | | | | | | | LOC100286922; LOC100286922; | | |
| | | | | | | | | LOC100286922 | | |
| cg16194588 | 4.21E−09 | 1.79E−03 | −0.262514348 | 0.262514348 | loss | 0.686031228 | 0.423516881 | LMTK3 | 37 | 19 |
| cg17641566 | 7.13E−10 | 3.03E−04 | −0.262513305 | 0.262513305 | loss | 0.785638134 | 0.523124829 | CDH23; CDH23; CDH23 | 37 | 10 |
| cg10817554 | 7.13E−10 | 3.03E−04 | −0.262491765 | 0.262491765 | loss | 0.513221142 | 0.250729376 | | 37 | 2 |
| cg20367961 | 8.47E−10 | 3.60E−04 | −0.262475418 | 0.262475418 | loss | 0.724463483 | 0.461988065 | EDN2; EDN2 | 37 | 1 |
| cg22352176 | 2.35E−09 | 9.98E−04 | −0.262473481 | 0.262473481 | loss | 0.673120234 | 0.410646753 | DISP1 | 37 | 1 |
| cg16179507 | 7.13E−10 | 3.03E−04 | −0.26244071 | 0.26244071 | loss | 0.916075557 | 0.653634847 | | 37 | 2 |
| cg20464732 | 7.84E−10 | 3.33E−04 | −0.262433178 | 0.262433178 | loss | 0.859876825 | 0.597443647 | | 37 | 8 |
| cg10249506 | 7.13E−10 | 3.03E−04 | −0.262429507 | 0.262429507 | loss | 0.961929713 | 0.699500206 | | 37 | 22 |
| cg07785717 | 1.01E−09 | 4.27E−04 | −0.262429281 | 0.262429281 | loss | 0.60941074 | 0.346981459 | CACNG8; MIR935 | 37 | 19 |
| cg12728621 | 7.13E−10 | 3.03E−04 | −0.262373743 | 0.262373743 | loss | 0.399779549 | 0.137405806 | EPB41L1; EPB41L1; EPB41L1; | 37 | 20 |
| | | | | | | | | EPB41L1; EPB41L1 | | |
| cg23515411 | 7.13E−10 | 3.03E−04 | −0.262295398 | 0.262295398 | loss | 0.626151457 | 0.363856059 | | 37 | 11 |
| cg23247955 | 2.35E−09 | 9.98E−04 | −0.262274444 | 0.262274444 | loss | 0.768299385 | 0.506024941 | LRRC59 | 37 | 17 |
| cg12281620 | 7.13E−10 | 3.03E−04 | −0.262248354 | 0.262248354 | loss | 0.896743072 | 0.634494718 | GNG7 | 37 | 19 |
| cg04495354 | 7.84E−10 | 3.33E−04 | −0.262243614 | 0.262243614 | loss | 0.514425673 | 0.252182059 | PLLP | 37 | 16 |
| cg17510121 | 7.13E−10 | 3.03E−04 | −0.262233548 | 0.262233548 | loss | 0.892215536 | 0.629981988 | SCUBE3 | 37 | 6 |
| cg13977320 | 7.13E−10 | 3.03E−04 | −0.262221739 | 0.262221739 | loss | 0.753598404 | 0.491376665 | KIF12 | 37 | 9 |
| cg03892457 | 3.28E−09 | 1.39E−03 | −0.262185415 | 0.262185415 | loss | 0.705102074 | 0.442916659 | CADM3; CADM3 | 37 | 1 |
| cg23487312 | 9.23E−10 | 3.92E−04 | −0.262180833 | 0.262180833 | loss | 0.510932698 | 0.248751865 | CADPS; CADPS; CADPS | 37 | 3 |
| cg01116161 | 7.13E−10 | 3.03E−04 | −0.26209294 | 0.26209294 | loss | 0.867644281 | 0.605551341 | | 37 | 5 |
| cg26133081 | 9.23E−10 | 3.92E−04 | −0.262070531 | 0.262070531 | loss | 0.89703136 | 0.634960829 | GJA3 | 37 | 13 |
| cg08577046 | 9.23E−10 | 3.92E−04 | −0.26205935 | 0.26205935 | loss | 0.838214779 | 0.576155429 | | 37 | 17 |
| cg21370924 | 7.13E−10 | 3.03E−04 | −0.262053278 | 0.262053278 | loss | 0.62912506 | 0.367071782 | ZBTB38 | 37 | 3 |
| cg06708213 | 7.13E−10 | 3.03E−04 | −0.262041336 | 0.262041336 | loss | 0.749299772 | 0.487258435 | MGAT58; MGAT58 | 37 | 17 |
| cg13163521 | 7.84E−10 | 3.33E−04 | −0.262029492 | 0.262029492 | loss | 0.366030979 | 0.104001486 | LTK; LTK; LTK | 37 | 15 |
| cg13275609 | 7.77E−10 | 3.30E−04 | −0.261938378 | 0.261938378 | loss | 0.780687313 | 0.518748935 | | 37 | 2 |
| cg17191443 | 7.13E−10 | 3.03E−04 | −0.261928639 | 0.261928639 | loss | 0.822427715 | 0.560499076 | MATN4; MATN4; MATN4 | 37 | 20 |
| cg03352287 | 1.10E−09 | 4.65E−04 | 0.261879537 | 0.261879537 | gain | 0.080196116 | 0.342075653 | CRCT1 | 37 | 1 |
| cg10750464 | 7.13E−10 | 3.03E−04 | −0.261872294 | 0.261872294 | loss | 0.496974694 | 0.2351024 | CCDC151 | 37 | 19 |
| cg06766541 | 7.13E−10 | 3.03E−04 | −0.261846986 | 0.261846986 | loss | 0.761107015 | 0.499260029 | | 37 | 7 |
| cg07795548 | 1.10E−09 | 4.65E−04 | −0.261803612 | 0.261803612 | loss | 0.891813853 | 0.630010241 | CYB5B | 37 | 16 |
| cg05766948 | 7.13E−10 | 3.03E−04 | −0.261795305 | 0.261795305 | loss | 0.32342806 | 0.061632755 | | 37 | 2 |
| cg10658779 | 7.13E−10 | 3.03E−04 | −0.261717185 | 0.261717185 | loss | 0.819760979 | 0.558043794 | C10orf11 | 37 | 10 |
| cg00255889 | 7.13E−10 | 3.03E−04 | −0.261691895 | 0.261691895 | loss | 0.678113077 | 0.416421182 | ATP8A2 | 37 | 13 |
| cg26584034 | 2.56E−09 | 1.09E−03 | −0.261663555 | 0.261663555 | loss | 0.559986308 | 0.298322753 | SYNPO; SYNPO | 37 | 5 |
| cg02455706 | 7.13E−10 | 3.03E−04 | −0.261633035 | 0.261633035 | loss | 0.429015174 | 0.167382138 | CRISPLD2 | 37 | 16 |
| cg08121453 | 7.13E−10 | 3.03E−04 | −0.261618561 | 0.261618561 | loss | 0.795095349 | 0.533476788 | | 37 | 1 |
| cg15927699 | 4.97E−09 | 2.11E−03 | −0.261607353 | 0.261607353 | loss | 0.678725506 | 0.417118153 | | 37 | 1 |
| cg05624199 | 1.01E−09 | 4.27E−04 | −0.26159131 | 0.26159131 | loss | 0.878825645 | 0.617234335 | | 37 | 19 |
| cg17267654 | 7.13E−10 | 3.03E−04 | −0.26157717 | 0.26157717 | loss | 0.93428987 | 0.6727127 | | 37 | 6 |
| cg20240715 | 9.23E−10 | 3.92E−04 | −0.26149994 | 0.26149994 | loss | 0.777204675 | 0.515704735 | KCNE1; KCNE1; KCNE1; KCNE1; | 37 | 21 |
| | | | | | | | | KCNE1; KCNE1; KCNE1; | | |
| | | | | | | | | KCNE1 | | |
| cg08709333 | 7.13E−10 | 3.03E−04 | −0.261499296 | 0.261499296 | loss | 0.832257543 | 0.570758247 | | 37 | 7 |
| cg22440848 | 4.97E−09 | 2.11E−03 | −0.261477119 | 0.261477119 | loss | 0.457829209 | 0.196352091 | | 37 | 4 |
| cg20814179 | 9.22E−08 | 3.91E−02 | −0.261476864 | 0.261476864 | loss | 0.559152628 | 0.297675765 | TMEM175 | 37 | 4 |
| cg16922753 | 2.21E−08 | 9.38E−03 | −0.261474497 | 0.261474497 | loss | 0.675035453 | 0.413560956 | | 37 | 8 |
| cg27312338 | 7.77E−10 | 3.30E−04 | −0.261444581 | 0.261444581 | loss | 0.797297628 | 0.535853047 | KIFC3; KIFC3; KIFC3 | 37 | 16 |
| cg26993965 | 3.88E−09 | 1.65E−03 | −0.261395994 | 0.261395994 | loss | 0.874527542 | 0.613131547 | | 37 | 18 |
| cg00800679 | 4.97E−09 | 2.11E−03 | −0.261367446 | 0.261367446 | loss | 0.758877217 | 0.497509771 | FGFBP1 | 37 | 4 |
| cg08985785 | 1.54E−08 | 6.56E−03 | −0.261340334 | 0.261340334 | loss | 0.595096923 | 0.333756588 | KCNK5 | 37 | 6 |
| cg15014458 | 9.23E−10 | 3.92E−04 | −0.261328263 | 0.261328263 | loss | 0.469430181 | 0.208101918 | LYPD3; LYPD3 | 37 | 19 |
| cg08260052 | 4.57E−09 | 1.94E−03 | −0.261313432 | 0.261313432 | loss | 0.754963391 | 0.493649959 | MEFV; MEFV | 37 | 16 |
| cg03020006 | 7.84E−10 | 3.33E−04 | −0.261305863 | 0.261305863 | loss | 0.958772504 | 0.697466641 | ALS2CR11; ALS2CR11; ALS2CR11; | 37 | 2 |
| | | | | | | | | ALS2CR11; ALS2CR11; | | |
| | | | | | | | | ALS2CR11; ALS2CR11; | | |
| | | | | | | | | ALS2CR11 | | |
| cg19730268 | 7.77E−10 | 3.30E−04 | −0.261299227 | 0.261299227 | loss | 0.745515051 | 0.484215824 | | 37 | 14 |
| cg04232360 | 7.13E−10 | 3.03E−04 | −0.261278392 | 0.261278392 | loss | 0.811841557 | 0.550563165 | | 37 | 1 |
| cg00648184 | 1.10E−09 | 4.65E−04 | −0.261257554 | 0.261257554 | loss | 0.967431177 | 0.706173624 | MARCH10; MARCH10; MIR548W | 37 | 17 |
| cg06769994 | 1.42E−09 | 6.01E−04 | −0.261244512 | 0.261244512 | loss | 0.579690047 | 0.318445535 | | 37 | 17 |
| cg02847897 | 1.19E−09 | 5.07E−04 | −0.261239183 | 0.261239183 | loss | 0.384647647 | 0.123408464 | KLHL8 | 37 | 4 |
| cg08777316 | 9.23E−10 | 3.92E−04 | −0.261234078 | 0.261234078 | loss | 0.380820019 | 0.119585941 | | 37 | 5 |
| cg05886689 | 7.13E−10 | 3.03E−04 | −0.261223689 | 0.261223689 | loss | 0.771231683 | 0.510007994 | LRRC4B | 37 | 19 |
| cg21199045 | 9.23E−10 | 3.92E−04 | −0.26120668 | 0.26120668 | loss | 0.797900898 | 0.536694218 | | 37 | 18 |
| cg16651537 | 9.23E−10 | 3.92E−04 | −0.261202981 | 0.261202981 | loss | 0.498129211 | 0.23692623 | CLEC11A | 37 | 19 |
| cg23257225 | 6.89E−09 | 2.93E−03 | −0.261172584 | 0.261172584 | loss | 0.712182766 | 0.451010182 | ADORA1; ADORA1 | 37 | 1 |
| cg08820801 | 7.13E−10 | 3.03E−04 | −0.261167139 | 0.261167139 | loss | 0.697386357 | 0.436219218 | FBXO17; FBXO17 | 37 | 19 |
| cg04872675 | 7.13E−10 | 3.03E−04 | −0.261164899 | 0.261164899 | loss | 0.789687781 | 0.528522882 | | 37 | 12 |
| cg25322720 | 8.47E−10 | 3.60E−04 | −0.261159613 | 0.261159613 | loss | 0.438091655 | 0.176932042 | | 37 | 17 |
| cg26122451 | 7.13E−10 | 3.03E−04 | −0.261095682 | 0.261095682 | loss | 0.45000947 | 0.188913788 | | 37 | 7 |
| cg26875637 | 9.23E−10 | 3.92E−04 | −0.26109558 | 0.26109558 | loss | 0.665165821 | 0.404070241 | CTTNBP2NL | 37 | 1 |
| cg12893736 | 7.13E−10 | 3.03E−04 | −0.261074228 | 0.261074228 | loss | 0.959119892 | 0.698045665 | GPRC5C; GPRC5C | 37 | 17 |
| cg08293242 | 8.80E−09 | 3.74E−03 | −0.261051246 | 0.261051246 | loss | 0.875102181 | 0.614050935 | | 37 | 13 |
| cg10589385 | 9.23E−10 | 3.92E−04 | −0.261008862 | 0.261008862 | loss | 0.623360762 | 0.3623519 | SETDB1; SETDB1; SETDB1 | 37 | 1 |
| cg09948179 | 1.73E−09 | 7.34E−04 | −0.261001536 | 0.261001536 | loss | 0.899160998 | 0.638159463 | PLA2G4E | 37 | 15 |
| cg10208897 | 2.16E−09 | 9.17E−04 | −0.260999734 | 0.260999734 | loss | 0.740620775 | 0.479621041 | ADAMTS2 | 37 | 5 |
| cg02311932 | 7.13E−10 | 3.03E−04 | −0.260998889 | 0.260998889 | loss | 0.461022283 | 0.200023394 | RBM24; RBM24; RBM24 | 37 | 6 |
| cg17930194 | 7.13E−10 | 3.03E−04 | −0.260953327 | 0.260953327 | loss | 0.320710892 | 0.059757565 | LHX6; LHX6; LHX6; LHX6; LHX6 | 37 | 9 |
| cg26840462 | 7.13E−10 | 3.03E−04 | −0.260947114 | 0.260947114 | loss | 0.959865085 | 0.698917971 | PKNOX2 | 37 | 11 |
| cg18264486 | 3.40E−08 | 1.44E−02 | −0.260938175 | 0.260938175 | loss | 0.478844143 | 0.217905968 | | 37 | 6 |
| cg01443452 | 8.47E−10 | 3.60E−04 | −0.26093022 | 0.26093022 | loss | 0.769206043 | 0.508275824 | GAPDH5 | 37 | 19 |
| cg16691177 | 7.13E−10 | 3.03E−04 | −0.260925896 | 0.260925896 | loss | 0.415557494 | 0.154631599 | TRIM61; TRIM61 | 37 | 4 |
| cg23916496 | 7.13E−10 | 3.03E−04 | −0.260906593 | 0.260906593 | loss | 0.635064923 | 0.375158329 | LRIT1 | 37 | 10 |
| cg01189915 | 7.13E−10 | 3.03E−04 | −0.260902924 | 0.260902924 | loss | 0.81123663 | 0.550333706 | HILPDA; HILPDA | 37 | 7 |
| cg17382918 | 1.10E−09 | 4.65E−04 | −0.260902649 | 0.260902649 | loss | 0.547646513 | 0.286743865 | APCDD1L | 37 | 20 |
| cg23093692 | 1.42E−09 | 6.01E−04 | −0.260895764 | 0.260895764 | loss | 0.782403323 | 0.521507559 | SLC35F3 | 37 | 1 |
| cg20996561 | 7.13E−10 | 3.03E−04 | −0.260890486 | 0.260890486 | loss | 0.435830651 | 0.174940165 | CACNA1I; CACNA1I | 37 | 22 |
| cg07498426 | 9.23E−10 | 3.92E−04 | −0.260887449 | 0.260887449 | loss | 0.947532002 | 0.686644553 | | 37 | 11 |
| cg04313735 | 2.06E−09 | 8.77E−04 | −0.260871843 | 0.260871843 | loss | 0.506597178 | 0.245725335 | PLEKHB1; PLEKHB1; PLEKHB1; | 37 | 11 |
| | | | | | | | | PLEKHB1 | | |
| cg18033416 | 7.13E−10 | 3.03E−04 | −0.260834684 | 0.260834684 | loss | 0.684353719 | 0.423519035 | RHBDL3 | 37 | 17 |
| cg26773791 | 7.13E−10 | 3.03E−04 | −0.2608205 | 0.2608205 | loss | 0.5865043 | 0.3256838 | | 37 | 11 |
| cg18385190 | 1.30E−09 | 5.52E−04 | −0.260797117 | 0.260797117 | loss | 0.653853358 | 0.393056241 | | 37 | 14 |
| cg06211243 | 2.48E−08 | 1.05E−02 | −0.260793459 | 0.260793459 | loss | 0.698553589 | 0.437760129 | | 37 | 18 |
| cg06378100 | 7.13E−10 | 3.03E−04 | −0.260777712 | 0.260777712 | loss | 0.574225183 | 0.313447471 | | 37 | 16 |
| cg21653641 | 7.13E−10 | 3.03E−04 | −0.260774307 | 0.260774307 | loss | 0.836986483 | 0.576212176 | MAMSTR; MAMSTR | 37 | 19 |
| cg14289461 | 7.13E−10 | 3.03E−04 | −0.260759352 | 0.260759352 | loss | 0.656400823 | 0.395641471 | LAMA4; LAMA4; LAMA4; LAMA4; | 37 | 6 |
| | | | | | | | | LAMA4; LAMA4; LAMA4; | | |
| | | | | | | | | LAMA4; LAMA4; LAMA4 | | |
| cg01266390 | 7.13E−10 | 3.03E−04 | −0.260758446 | 0.260758446 | loss | 0.589335775 | 0.328577329 | | 37 | 10 |
| cg04131405 | 7.13E−10 | 3.03E−04 | −0.260756539 | 0.260756539 | loss | 0.913214745 | 0.652458206 | DLX4; DLX4 | 37 | 17 |
| cg12416033 | 7.13E−10 | 3.03E−04 | −0.260746827 | 0.260746827 | loss | 0.760271657 | 0.499524829 | SDHAF1 | 37 | 19 |
| cg16851282 | 5.39E−09 | 2.29E−03 | −0.260681899 | 0.260681899 | loss | 0.900483417 | 0.639801518 | MRGPRE | 37 | 11 |
| cg06784539 | 7.13E−10 | 3.03E−04 | −0.260654871 | 0.260654871 | loss | 0.757367936 | 0.496713065 | TMEM213; ATP6V0A4; ATP6V0A4; | 37 | 7 |
| | | | | | | | | ATP6V0A4; ATP6V0A4 | | |
| cg11909574 | 1.54E−09 | 6.54E−04 | −0.260637107 | 0.260637107 | loss | 0.702472966 | 0.441835859 | | 37 | 11 |
| cg09884310 | 6.32E−09 | 2.68E−03 | −0.260630808 | 0.260630808 | loss | 0.813091483 | 0.552460675 | | 37 | 6 |
| cg16658020 | 9.23E−10 | 3.92E−04 | −0.260623444 | 0.260623444 | loss | 0.784281232 | 0.523657788 | KRTAP2-1; KRTAP2-1 | 37 | 17 |
| cg04259752 | 7.13E−10 | 3.03E−04 | −0.260577511 | 0.260577511 | loss | 0.697251017 | 0.436673506 | LOXL2 | 37 | 8 |
| cg01268571 | 1.43E−08 | 6.06E−03 | −0.26054198 | 0.26054198 | loss | 0.412242209 | 0.151700229 | | 37 | 2 |
| cg25249286 | 5.39E−09 | 2.29E−03 | −0.260454373 | 0.260454373 | loss | 0.751343743 | 0.490889371 | | 37 | 2 |
| cg21313071 | 1.10E−09 | 4.65E−04 | −0.260449188 | 0.260449188 | loss | 0.750263594 | 0.489814406 | KCNG1 | 37 | 20 |
| cg21248196 | 8.47E−10 | 3.60E−04 | −0.260344189 | 0.260344189 | loss | 0.774164125 | 0.513819935 | PSMA8; PSMA8; PSMA8 | 37 | 18 |
| cg26688911 | 7.13E−10 | 3.03E−04 | −0.260341 | 0.260341 | loss | 0.447027506 | 0.186686505 | LPIN3 | 37 | 20 |
| cg25217583 | 8.56E−10 | 3.63E−04 | −0.260336174 | 0.260336174 | loss | 0.816116904 | 0.555780729 | ELAVL4; ELAVL4; ELAVL4; ELAVL4; | 37 | 1 |
| | | | | | | | | ELAVL4 | | |
| cg08802482 | 9.23E−10 | 3.92E−04 | −0.260330974 | 0.260330974 | loss | 0.640137562 | 0.379806588 | | 37 | 7 |
| cg13673514 | 7.13E−10 | 3.03E−04 | −0.260318713 | 0.260318713 | loss | 0.49662813 | 0.236309418 | BEGAIN; BEGAIN | 37 | 14 |
| cg20764575 | 7.13E−10 | 3.03E−04 | −0.26029711 | 0.26029711 | loss | 0.666813198 | 0.406516088 | DFNA5; DFNA5; DFNA5 | 37 | 7 |
| cg08836375 | 3.97E−08 | 1.69E−02 | −0.260265393 | 0.260265393 | loss | 0.96899584 | 0.708730447 | | 37 | 6 |
| cg08420135 | 7.13E−10 | 3.03E−04 | −0.260226531 | 0.260226531 | loss | 0.883486272 | 0.623259741 | | 37 | 12 |
| cg10528576 | 1.39E−08 | 5.90E−03 | −0.260165415 | 0.260165415 | loss | 0.55219205 | 0.292026635 | DLK1 | 37 | 14 |
| cg18242139 | 1.01E−09 | 4.27E−04 | −0.260089327 | 0.260089327 | loss | 0.800479421 | 0.540390094 | ELAVL4; ELAVL4; ELAVL4; ELAVL4; | 37 | 1 |
| | | | | | | | | ELAVL4 | | |
| cg19380499 | 7.13E−10 | 3.03E−04 | −0.260083493 | 0.260083493 | loss | 0.362706694 | 0.102623201 | EFCC1 | 37 | 3 |
| cg00009053 | 7.13E−10 | 3.03E−04 | −0.260076835 | 0.260076835 | loss | 0.630707411 | 0.370630576 | SYT7; SYT7 | 37 | 11 |
| cg24015889 | 1.19E−09 | 5.07E−04 | −0.260075464 | 0.260075454 | loss | 0.665298311 | 0.405222847 | | 37 | 17 |
| cg00700487 | 7.13E−10 | 3.03E−04 | −0.260030894 | 0.260030894 | loss | 0.578001847 | 0.317970953 | NOS3 | 37 | 7 |
| cg08121652 | 1.01E−09 | 4.27E−04 | −0.260024648 | 0.260024648 | loss | 0.720224119 | 0.460199471 | TRABD2B | 37 | 1 |
| cg22398616 | 7.13E−10 | 3.03E−04 | −0.25994321 | 0.25994321 | loss | 0.766951051 | 0.507007841 | LECT1; LECT1 | 37 | 13 |
| cg12468238 | 9.23E−10 | 3.92E−04 | −0.259922757 | 0.259922757 | loss | 0.720347951 | 0.460425194 | IRX1 | 37 | 5 |
| cg13622265 | 8.47E−10 | 3.60E−04 | −0.259897402 | 0.259897402 | loss | 0.887826391 | 0.627928988 | TTLL11 | 37 | 9 |
| cg16987606 | 7.13E−10 | 3.03E−04 | −0.259888784 | 0.259888784 | loss | 0.355362421 | 0.095473637 | GPRC5C | 37 | 17 |
| cg26312935 | 1.01E−09 | 4.27E−04 | −0.259850454 | 0.259850454 | loss | 0.645286625 | 0.385436171 | RNF111; RNF111; RNF111; | 37 | 15 |
| | | | | | | | | RNF111 | | |
| cg16715722 | 7.13E−10 | 3.03E−04 | −0.259801546 | 0.259801546 | loss | 0.347053834 | 0.587252288 | TLX1NB; TLX1; TLX1 | 37 | 10 |
| cg12793096 | 3.67E−08 | 1.56E−02 | −0.259798193 | 0.259798193 | loss | 0.90437384 | 0.644575647 | | 37 | 6 |
| cg13475995 | 7.13E−10 | 3.03E−04 | −0.259741229 | 0.259741229 | loss | 0.6755456 | 0.415804371 | | 37 | 10 |
| cg16813662 | 7.13E−10 | 3.03E−04 | −0.259736002 | 0.259736002 | loss | 0.559081802 | 0.2993458 | LINC00554 | 37 | 13 |
| cg03907016 | 7.77E−10 | 3.30E−04 | −0.259703211 | 0.259703211 | loss | 0.891260164 | 0.631556953 | TNFSF9 | 37 | 19 |
| cg10461261 | 7.13E−10 | 3.03E−04 | −0.259671152 | 0.259571152 | loss | 0.827176217 | 0.567505065 | | 37 | 15 |
| cg07195674 | 7.13E−10 | 3.03E−04 | −0.259632674 | 0.259632674 | loss | 0.862818504 | 0.603185829 | | 37 | 14 |
| cg17181653 | 7.13E−10 | 3.03E−04 | −0.259624636 | 0.259624636 | loss | 0.553813583 | 0.294188947 | SF3B5 | 37 | 6 |
| cg02475301 | 1.42E−09 | 6.01E−04 | −0.259584758 | 0.259584758 | loss | 0.81168237 | 0.552097612 | MRGPRE | 37 | 11 |
| cg12672189 | 1.96E−08 | 8.33E−03 | −0.259552204 | 0.259552204 | loss | 0.59106614 | 0.331513935 | | 37 | 6 |
| cg18049167 | 7.13E−10 | 3.03E−04 | −0.259545728 | 0.259545728 | loss | 0.353842715 | 0.09429698 | PPT2; PPT2; PPT2; LOC100507547; | 37 | 6 |
| | | | | | | | | LOC100507547; LOC100507547; | | |
| | | | | | | | | LOC100507547; | | |
| | | | | | | | | PPT2-EGFL8 | | |
| cg03890167 | 7.13E−10 | 3.03E−04 | −0.259538631 | 0.259538631 | loss | 0.425562872 | 0.166024241 | | 37 | 1 |
| cg16454130 | 8.47E−10 | 3.60E−04 | −0.259533777 | 0.259533777 | loss | 0.791947777 | 0.532414 | CLDN4 | 37 | 7 |
| cg04683509 | 1.10E−09 | 4.65E−04 | −0.259520172 | 0.259520172 | loss | 0.861649666 | 0.602129494 | PPP1R13L; PPP1R13L | 37 | 19 |
| cg22694931 | 7.13E−10 | 3.03E−04 | −0.259492924 | 0.259492924 | loss | 0.856335077 | 0.596842153 | DACT2 | 37 | 6 |
| cg20149170 | 7.13E−10 | 3.03E−04 | −0.259421281 | 0.259421281 | loss | 0.88740054 | 0.627979259 | EPHB2; EPHB2 | 37 | 1 |
| cg01690119 | 7.95E−16 | 3.38E−10 | −0.25942123 | 0.25942123 | loss | 0.714779412 | 0.455358182 | | 37 | 16 |
| cg04964333 | 7.13E−10 | 3.03E−04 | −0.259420198 | 0.259420198 | loss | 0.776829704 | 0.517409506 | LPIN3 | 37 | 20 |
| cg23058194 | 7.13E−10 | 3.03E−04 | −0.25927627 | 0.25927627 | loss | 0.930510653 | 0.671234382 | KBTBD13 | 37 | 15 |
| cg00771642 | 7.84E−10 | 3.33E−04 | −0.259268721 | 0.259268721 | loss | 0.957894127 | 0.698625406 | | 37 | 19 |
| cg19497998 | 7.13E−10 | 3.03E−04 | −0.259249094 | 0.259249094 | loss | 0.593012094 | 0.333763 | | 37 | 6 |
| cg11364888 | 8.47E−10 | 3.60E−04 | −0.259242945 | 0.259242945 | loss | 0.913625151 | 0.654382206 | | 37 | 12 |
| cg16440561 | 1.10E−09 | 4.65E−04 | −0.259239094 | 0.259239094 | loss | 0.300212595 | 0.040973501 | SPEG | 37 | 2 |
| cg10137597 | 1.10E−09 | 4.65E−04 | −0.259162392 | 0.259162392 | loss | 0.750302545 | 0.491140153 | PIWIL2; PIWIL2 | 37 | 8 |
| cg26530345 | 9.23E−10 | 3.92E−04 | −0.259160212 | 0.259160212 | loss | 0.866409653 | 0.607249441 | SPEG | 37 | 2 |
| cg07740110 | 7.84E−10 | 3.33E−04 | −0.259149897 | 0.259149897 | loss | 0.578819833 | 0.319669935 | TRPV3; TRPV3 | 37 | 17 |
| cg09103073 | 7.13E−10 | 3.03E−04 | −0.259115162 | 0.259115162 | loss | 0.893388709 | 0.634273547 | KRT12 | 37 | 17 |
| cg16405908 | 2.35E−09 | 9.98E−04 | −0.259099591 | 0.259099591 | loss | 0.892033668 | 0.632934076 | MRGPRE | 37 | 11 |
| cg08661360 | 7.13E−10 | 3.03E−04 | −0.259076677 | 0.259076677 | loss | 0.630152283 | 0.371075606 | TNXB | 37 | 6 |
| cg03555881 | 2.86E−09 | 1.22E−03 | −0.259051964 | 0.259051964 | loss | 0.872480546 | 0.613428582 | | 37 | 6 |
| cg24728050 | 1.01E−09 | 4.27E−04 | −0.259012944 | 0.259012944 | loss | 0.881884485 | 0.622871541 | MRGPRE; MRGPRE | 37 | 11 |
| cg11043571 | 1.96E−08 | 8.33E−03 | −0.259003484 | 0.259003484 | loss | 0.862009966 | 0.603006482 | CPE | 37 | 4 |
| cg15899727 | 1.10E−09 | 4.65E−04 | −0.258995863 | 0.258995863 | loss | 0.744769287 | 0.485773424 | | 37 | 1 |
| cg07688931 | 9.23E−10 | 3.92E−04 | −0.25899373 | 0.25899373 | loss | 0.786938089 | 0.527944359 | | 37 | 5 |
| cg19659004 | 2.78E−09 | 1.18E−03 | −0.258966495 | 0.258966495 | loss | 0.437567611 | 0.178601116 | AMN | 37 | 14 |
| cg14223654 | 3.57E−09 | 1.52E−03 | −0.258904869 | 0.258904869 | loss | 0.54277024 | 0.283865371 | | 37 | 2 |
| cg06720467 | 7.13E−10 | 3.03E−04 | −0.258888921 | 0.258888921 | loss | 0.545760762 | 0.286871841 | SFN | 37 | 1 |
| cg26304689 | 7.13E−10 | 3.03E−04 | −0.258868245 | 0.258868245 | loss | 0.764171951 | 0.505303706 | | 37 | 5 |
| cg09159285 | 7.13E−10 | 3.03E−04 | −0.25886042 | 0.25886042 | loss | 0.315609408 | 0.056748988 | KLHL21 | 37 | 1 |
| cg19702802 | 7.13E−10 | 3.03E−04 | −0.258818911 | 0.258818911 | loss | 0.60918367 | 0.350364759 | NANOS2 | 37 | 19 |
| cg09842196 | 8.47E−10 | 3.60E−04 | −0.258767253 | 0.258767253 | loss | 0.802335106 | 0.543567853 | | 37 | 13 |
| cg05079544 | 8.47E−10 | 3.60E−04 | −0.25874082 | 0.25874082 | loss | 0.669660096 | 0.410919276 | N4BP1 | 37 | 16 |
| cg16653901 | 7.13E−10 | 3.03E−04 | −0.258734312 | 0.258734312 | loss | 0.945585847 | 0.686851535 | SKP1; SKP1 | 37 | 5 |
| cg05530630 | 7.13E−10 | 3.03E−04 | −0.258731002 | 0.258731002 | loss | 0.729966408 | 0.471235406 | EGFR; EGFR; EGFR | 37 | 7 |
| cg17820828 | 3.02E−09 | 1.28E−03 | −0.258726051 | 0.258726051 | loss | 0.575047304 | 0.316321253 | KCNQ1; KCNQ1 | 37 | 11 |
| cg07008193 | 7.13E−10 | 3.03E−04 | −0.258601263 | 0.258601263 | loss | 0.473387092 | 0.214785829 | CREB3L1 | 37 | 11 |
| cg01917340 | 9.23E−10 | 3.92E−04 | −0.258595611 | 0.258595611 | loss | 0.662701911 | 0.4041063 | | 37 | 14 |
| cg00455331 | 7.13E−10 | 3.03E−04 | −0.258590751 | 0.258590751 | loss | 0.793269298 | 0.534678547 | | 37 | 2 |
| cg05543030 | 1.01E−09 | 4.27E−04 | −0.258585277 | 0.258585277 | loss | 0.534828583 | 0.276243306 | | 37 | 2 |
| cg09826056 | 1.10E−09 | 4.65E−04 | −0.258488117 | 0.258488117 | loss | 0.432536375 | 0.174048259 | GIPC2 | 37 | 1 |
| cg06691250 | 3.14E−08 | 1.33E−02 | −0.25848391 | 0.25848391 | loss | 0.724570292 | 0.466086382 | | 37 | 10 |
| cg05329578 | 7.77E−10 | 3.30E−04 | −0.258411189 | 0.258411189 | loss | 0.356119092 | 0.097707904 | | 37 | 9 |
| cg16107528 | 8.47E−10 | 3.60E−04 | −0.258407816 | 0.258407816 | loss | 0.804019287 | 0.545611471 | NTN1 | 37 | 17 |
| cg01095004 | 7.13E−10 | 3.03E−04 | −0.258357443 | 0.258357443 | loss | 0.973243408 | 0.714885965 | | 37 | 16 |
| cg05096467 | 1.03E−08 | 4.39E−03 | −0.258314807 | 0.258314807 | loss | 0.403931902 | 0.145617095 | TEX14; TEX14; TEX14 | 37 | 17 |
| cg00169782 | 7.13E−10 | 3.03E−04 | −0.25831348 | 0.25831348 | loss | 0.826698568 | 0.568385088 | KRT42P | 37 | 17 |
| cg01965673 | 7.13E−10 | 3.03E−04 | −0.258274903 | 0.258274903 | loss | 0.676207532 | 0.417932629 | | 37 | 3 |
| cg23591611 | 7.13E−10 | 3.03E−04 | −0.258267547 | 0.258267547 | loss | 0.512039406 | 0.253771859 | | 37 | 12 |
| cg17590162 | 7.13E−10 | 3.03E−04 | −0.258258157 | 0.258258157 | loss | 0.537720057 | 0.2794619 | MACROD1 | 37 | 11 |
| cg19307528 | 7.13E−10 | 3.03E−04 | −0.258247775 | 0.258247775 | loss | 0.703898175 | 0.4456504 | SBSPON | 37 | 8 |
| cg25530735 | 1.68E−09 | 7.12E−04 | −0.258233386 | 0.258233386 | loss | 0.884333704 | 0.626100318 | | 37 | 5 |
| cg24880736 | 7.13E−10 | 3.03E−04 | −0.258196614 | 0.258196614 | loss | 0.663735155 | 0.405538541 | | 37 | 7 |
| cg22033281 | 6.35E−09 | 2.70E−03 | −0.258173382 | 0.258173382 | loss | 0.554613717 | 0.296440335 | | 37 | 11 |
| cg09201238 | 7.13E−10 | 3.03E−04 | −0.258172094 | 0.258172094 | loss | 0.686170847 | 0.427998753 | | 37 | 17 |
| cg21291672 | 7.13E−10 | 3.03E−04 | −0.258168422 | 0.258168422 | loss | 0.543565534 | 0.285397112 | TMEM200B | 37 | 1 |
| cg16071029 | 1.96E−08 | 8.33E−03 | −0.258157897 | 0.258157897 | loss | 0.537304208 | 0.279146311 | | 37 | 6 |
| cg08175536 | 1.19E−09 | 5.07E−04 | −0.258145475 | 0.258145475 | loss | 0.765261734 | 0.507116259 | LINC00925 | 37 | 15 |
| cg01114163 | 2.91E−08 | 1.23E−02 | −0.258113591 | 0.258113591 | loss | 0.749813785 | 0.491700194 | | 37 | 5 |
| cg09511741 | 1.68E−09 | 7.12E−04 | −0.258081375 | 0.258081375 | loss | 0.89590484 | 0.637823465 | TRPM4; TRPM4 | 37 | 19 |
| cg16154524 | 7.13E−10 | 3.03E−04 | −0.25805147 | 0.25805147 | loss | 0.819376858 | 0.561325388 | KRT18P55 | 37 | 17 |
| cg13734535 | 7.13E−10 | 3.03E−04 | −0.258022381 | 0.258022381 | loss | 0.797886892 | 0.539864512 | NCS1 | 37 | 9 |
| cg26572207 | 7.13E−10 | 3.03E−04 | −0.258005095 | 0.258005095 | loss | 0.62113653 | 0.363131435 | | 37 | 8 |
| cg10132767 | 2.48E−08 | 1.05E−02 | −0.257952363 | 0.257952363 | loss | 0.785682387 | 0.527730024 | | 37 | 6 |
| cg07814122 | 5.41E−08 | 2.30E−02 | −0.257909327 | 0.257909327 | loss | 0.797239051 | 0.539329724 | KIAA1467 | 37 | 12 |
| cg10799440 | 7.13E−10 | 3.03E−04 | −0.257769295 | 0.257769295 | loss | 0.827491466 | 0.569722171 | | 37 | 8 |
| cg04781339 | 8.47E−10 | 3.60E−04 | −0.257760807 | 0.257760807 | loss | 0.56140106 | 0.303640253 | | 37 | 21 |
| cg06949812 | 7.13E−10 | 3.03E−04 | −0.257740193 | 0.257740193 | loss | 0.421662481 | 0.163922288 | GJD3 | 37 | 17 |
| cg13695646 | 7.13E−10 | 3.03E−04 | −0.257726796 | 0.257726796 | loss | 0.520765291 | 0.263038494 | BHMT | 37 | 5 |
| cg12458966 | 7.13E−10 | 3.03E−04 | −0.257701564 | 0.257701564 | loss | 0.750371175 | 0.492669612 | | 37 | 1 |
| cg05804139 | 3.28E−09 | 1.39E−03 | −0.257699951 | 0.257699951 | loss | 0.764248209 | 0.506548259 | | 37 | 6 |
| cg04986504 | 7.13E−10 | 3.03E−04 | −0.257692425 | 0.257692425 | loss | 0.478901719 | 0.221209294 | DKK3; DKK3; DKK3 | 37 | 11 |
| cg03492827 | 7.13E−10 | 3.03E−04 | −0.257688737 | 0.257688737 | loss | 0.408553519 | 0.150864782 | | 37 | 7 |
| cg07923390 | 7.13E−10 | 3.03E−04 | −0.25767456 | 0.25767456 | loss | 0.75966366 | 0.5019891 | TCP11; TCP11; TCP11; TCP11; | 37 | 6 |
| | | | | | | | | TCP11; TCP11; TCP11; TCP11; | | |
| | | | | | | | | TCP11; TCP11; TCP11; | | |
| | | | | | | | | TCP11; TCP11; TCP11 | | |
| cg22138002 | 7.13E−10 | 3.03E−04 | −0.257617781 | 0.257617781 | loss | 0.803812834 | 0.546195053 | LOXHD1; LOXHD1 | 37 | 18 |
| cg21615654 | 3.02E−09 | 1.28E−03 | −0.257595113 | 0.257595113 | loss | 0.851828219 | 0.594233106 | | 37 | 5 |
| cg11985839 | 1.80E−12 | 7.66E−07 | −0.257582899 | 0.257582899 | loss | 0.544955076 | 0.287372177 | DDR1; DDR1; DDR1 | 37 | 6 |
| cg25923214 | 7.13E−10 | 3.03E−04 | −0.257564569 | 0.257564569 | loss | 0.431429081 | 0.173864512 | MGAT5B; MGAT5B | 37 | 17 |
| cg08031492 | 7.84E−10 | 3.33E−04 | −0.257557457 | 0.257557457 | loss | 0.636952569 | 0.379395112 | | 37 | 2 |
| cg10265365 | 7.13E−10 | 3.03E−04 | −0.257511509 | 0.257511509 | loss | 0.707392426 | 0.449880918 | TMEM145 | 37 | 19 |
| cg24371114 | 5.85E−09 | 2.48E−03 | −0.257499586 | 0.257499586 | loss | 0.677922375 | 0.420422788 | CCDC149; CCDC149 | 37 | 4 |
| cg10455971 | 7.95E−08 | 3.37E−02 | −0.257482699 | 0.257482699 | loss | 0.639033746 | 0.381551047 | | 37 | 5 |
| cg05551068 | 7.13E−10 | 3.03E−04 | −0.257457915 | 0.257457915 | loss | 0.37988357 | 0.122425655 | RFC5; RFC5; RFC5; RFC5 | 37 | 12 |
| cg14942906 | 1.54E−09 | 6.54E−04 | −0.25744127 | 0.25744127 | loss | 0.648564653 | 0.391123382 | DOC2GP | 37 | 11 |
| cg15015426 | 1.19E−09 | 5.07E−04 | −0.257437364 | 0.257437364 | loss | 0.596009217 | 0.338571853 | OR10J5 | 37 | 1 |
| cg01568317 | 7.13E−10 | 3.03E−04 | −0.257436517 | 0.257436517 | loss | 0.799347811 | 0.541911294 | | 37 | 14 |
| cg12987312 | 2.16E−09 | 9.17E−04 | −0.257425579 | 0.257425579 | loss | 0.610597474 | 0.353171894 | | 37 | 3 |
| cg04321555 | 1.68E−09 | 7.12E−04 | −0.257409522 | 0.257409522 | loss | 0.638728645 | 0.381319124 | | 37 | 19 |
| cg03987884 | 8.11E−09 | 3.44E−03 | −0.257391994 | 0.257391994 | loss | 0.852861506 | 0.595469512 | HMSD | 37 | 18 |
| cg01444808 | 1.30E−09 | 5.52E−04 | −0.257385099 | 0.257385099 | loss | 0.784548487 | 0.527163388 | SEPT8; SEPT8; SEPT8; SEPT8 | 37 | 5 |
| cg20078406 | 1.10E−09 | 4.65E−04 | −0.257368313 | 0.257368313 | loss | 0.899497113 | 0.6421288 | PRSS42; PRSS42 | 37 | 3 |
| cg04120272 | 1.19E−09 | 5.07E−04 | −0.257219526 | 0.257219526 | loss | 0.412975325 | 0.155755798 | FBXO47; FBXO47 | 37 | 17 |
| cg16425726 | 5.41E−08 | 2.30E−02 | −0.257191352 | 0.257191352 | loss | 0.962054381 | 0.704863029 | SCD5; SCD5 | 37 | 4 |
| cg04471507 | 7.13E−10 | 3.03E−04 | −0.257178032 | 0.257178032 | loss | 0.350577238 | 0.093399206 | STMN1; STMN1; STMN1; STMN1; | 37 | 1 |
| | | | | | | | | STMN1; MIR3917 | | |
| cg26636010 | 1.19E−09 | 5.07E−04 | −0.257161149 | 0.257161149 | loss | 0.567610355 | 0.310449206 | | 37 | 2 |
| cg27194211 | 7.13E−10 | 3.03E−04 | −0.257139016 | 0.257139016 | loss | 0.729002581 | 0.471863565 | SMTNL2; SMTNL2 | 37 | 17 |
| cg03638715 | 7.13E−10 | 3.03E−04 | −0.257139011 | 0.257139011 | loss | 0.829396275 | 0.572257265 | UBALD1 | 37 | 16 |
| cg01806151 | 7.13E−10 | 3.03E−04 | −0.257130947 | 0.257130947 | loss | 0.7170639 | 0.459932953 | | 37 | 1 |
| cg15344726 | 1.19E−09 | 5.07E−04 | −0.25712431 | 0.25712431 | loss | 0.847134757 | 0.590060447 | GJA3 | 37 | 13 |
| cg10279314 | 2.56E−09 | 1.09E−03 | −0.257092567 | 0.257092567 | loss | 0.596140349 | 0.339047782 | | 37 | 6 |
| cg23789054 | 7.77E−10 | 3.30E−04 | −0.257084476 | 0.257084476 | loss | 0.87652437 | 0.619439894 | LOC401242 | 37 | 6 |
| cg09033333 | 4.57E−09 | 1.94E−03 | −0.257038705 | 0.257038706 | loss | 0.594834047 | 0.337795341 | | 37 | 10 |
| cg08414643 | 1.03E−08 | 4.39E−03 | −0.25698464 | 0.25698464 | loss | 0.402967104 | 0.145982464 | | 37 | 5 |
| cg22952917 | 1.10E−09 | 4.65E−04 | −0.25696396 | 0.25596396 | loss | 0.842029219 | 0.585065259 | NOX5; NOX5; NOX5; MIR548H4; | 37 | 15 |
| | | | | | | | | NOX5; NOX5 | | |
| cg03800939 | 7.77E−10 | 3.30E−04 | −0.256938935 | 0.256938935 | loss | 0.466492247 | 0.209553312 | SLC37A2; SLC37A2 | 37 | 11 |
| cg22478544 | 7.13E−10 | 3.03E−04 | −0.256925187 | 0.256925187 | loss | 0.661976087 | 0.4050509 | | 37 | 5 |
| cg11747081 | 1.22E−08 | 5.16E−03 | −0.256910214 | 0.256910214 | loss | 0.795127485 | 0.538217271 | C13orf45 | 37 | 13 |
| cg21488617 | 7.13E−10 | 3.03E−04 | −0.25690849 | 0.25690849 | loss | 0.482870466 | 0.225961976 | DNALI1 | 37 | 1 |
| cg18717329 | 7.77E−10 | 3.30E−04 | −0.256900548 | 0.256900548 | loss | 0.892419289 | 0.635518741 | | 37 | 14 |
| cg06470855 | 1.22E−08 | 5.16E−03 | −0.256874868 | 0.256874868 | loss | 0.692114121 | 0.435239253 | | 37 | 13 |
| cg09556447 | 1.01E−09 | 4.27E−04 | −0.256839596 | 0.256839596 | loss | 0.93262736 | 0.675787765 | PI16; PI16 | 37 | 6 |
| cg14701108 | 3.67E−08 | 1.56E−02 | −0.25680821 | 0.25680821 | loss | 0.612209257 | 0.355401047 | | 37 | 2 |
| cg05708536 | 7.84E−10 | 3.33E−04 | −0.25675342 | 0.25675342 | loss | 0.548016679 | 0.291263259 | | 37 | 1 |
| cg09576762 | 7.13E−10 | 3.03E−04 | −0.256735067 | 0.256735067 | loss | 0.590578979 | 0.333843912 | PGPEP1L; PGPEP1L; PGPEP1L | 37 | 15 |
| cg14637144 | 8.47E−10 | 3.60E−04 | −0.256703862 | 0.256703862 | loss | 0.440158957 | 0.183455094 | MFI2; MFI2 | 37 | 3 |
| cg01759672 | 7.13E−10 | 3.03E−04 | −0.256699851 | 0.256699851 | loss | 0.98213694 | 0.725437088 | GFRA3 | 37 | 5 |
| cg04017036 | 9.23E−10 | 3.92E−04 | −0.256686978 | 0.256686978 | loss | 0.867275849 | 0.610588871 | | 37 | 16 |
| cg02888048 | 3.14E−08 | 1.33E−02 | −0.256673586 | 0.256673586 | loss | 0.492821162 | 0.236147576 | | 37 | 7 |
| cg25470758 | 8.65E−10 | 3.67E−04 | −0.256668004 | 0.256668004 | loss | 0.736976739 | 0.480308735 | | 37 | 1 |
| cg10313047 | 8.47E−10 | 3.60E−04 | −0.256667007 | 0.256667007 | loss | 0.553387719 | 0.296720712 | DNAH1 | 37 | 3 |
| cg25011257 | 7.13E−10 | 3.03E−04 | −0.256664127 | 0.256664127 | loss | 0.712457104 | 0.455792976 | TH; TH; TH; TH; TH; TH; MIR4686 | 37 | 11 |
| cg10409299 | 1.83E−09 | 7.75E−04 | −0.256646796 | 0.256646796 | loss | 0.673017043 | 0.416370247 | TMEM176B; TMEM176B; | 37 | 7 |
| | | | | | | | | TMEM176B; TMEM176B; | | |
| | | | | | | | | TMEM176A | | |
| cg03444077 | 7.13E−10 | 3.03E−04 | −0.25662586 | 0.25662586 | loss | 0.530373113 | 0.253747253 | TMEM72-AS1 | 37 | 10 |
| cg21761639 | 1.42E−09 | 6.01E−04 | −0.256592959 | 0.256592959 | loss | 0.530811153 | 0.274218194 | | 37 | 7 |
| cg14476358 | 7.13E−10 | 3.03E−04 | −0.256569353 | 0.256569353 | loss | 0.542704353 | 0.286135 | | 37 | 9 |
| cg13422817 | 9.23E−10 | 3.92E−04 | −0.256566282 | 0.256566282 | loss | 0.62376777 | 0.367201488 | FGF6 | 37 | 12 |
| cg03238581 | 7.13E−10 | 3.03E−04 | −0.256560444 | 0.256560444 | loss | 0.745688621 | 0.489128176 | CSPG4 | 37 | 15 |
| cg05928980 | 7.13E−10 | 3.03E−04 | −0.256522984 | 0.256522984 | loss | 0.81381056 | 0.557287576 | PTPRU; PTPRU; PTPRU; PTPRU | 37 | 1 |
| cg05728902 | 7.13E−10 | 3.03E−04 | −0.256474747 | 0.256474747 | loss | 0.713850211 | 0.457375465 | INHBE | 37 | 12 |
| cg21607649 | 7.13E−10 | 3.03E−04 | −0.256465509 | 0.256465509 | loss | 0.712293879 | 0.455828371 | | 37 | 10 |
| cg26077005 | 7.13E−10 | 3.03E−04 | −0.256460309 | 0.256460309 | loss | 0.618830679 | 0.362370371 | | 37 | 6 |
| cg05517791 | 1.10E−09 | 4.65E−04 | −0.256459001 | 0.256459001 | loss | 0.657721077 | 0.401262076 | | 37 | 7 |
| cg14020904 | 7.13E−10 | 3.03E−04 | −0.256396206 | 0.256396206 | loss | 0.738006642 | 0.481610435 | C1QTNF1; C1QTNF1; C1QTNF1; | 37 | 17 |
| | | | | | | | | C1QTNF1- | | |
| | | | | | | | | AS1; C1QTNF1-AS1 | | |
| cg11625488 | 7.13E−10 | 3.03E−04 | 0.25637507 | 0.25637507 | gain | 0.437525842 | 0.693900912 | | 37 | 6 |
| cg07803420 | 7.13E−10 | 3.03E−04 | −0.256364618 | 0.256364618 | loss | 0.7201006 | 0.463735982 | DDR1; DDR1; DDR1 | 37 | 6 |
| cg08370430 | 8.47E−10 | 3.60E−04 | −0.256345287 | 0.256345287 | loss | 0.833634781 | 0.577289494 | | 37 | 17 |
| cg05338187 | 7.77E−10 | 3.30E−04 | −0.256276454 | 0.256276454 | loss | 0.62420323 | 0.367926776 | LGALS8; LGALS8; LGALS8; LGALS8; | 37 | 1 |
| | | | | | | | | LGALS8-AS1 | | |
| cg00555085 | 7.13E−10 | 3.03E−04 | −0.256229247 | 0.256229247 | loss | 0.860075047 | 0.6038458 | | 37 | 16 |
| cg09468051 | 7.13E−10 | 3.03E−04 | −0.256192375 | 0.256192375 | loss | 0.671315398 | 0.415123024 | | 37 | 4 |
| cg05653018 | 7.13E−10 | 3.03E−04 | −0.256121433 | 0.256121433 | loss | 0.860440057 | 0.604318624 | ELF3; ELF3 | 37 | 1 |
| cg27008363 | 7.13E−10 | 3.03E−04 | −0.256120241 | 0.256120241 | loss | 0.883587758 | 0.627467518 | HOXC4; HOXC4 | 37 | 12 |
| cg05628160 | 3.88E−09 | 1.65E−03 | −0.256115495 | 0.256115495 | loss | 0.801069719 | 0.544954224 | RGS20; RGS20; RG520; RGS20 | 37 | 8 |
| cg08000731 | 1.89E−09 | 8.02E−04 | −0.256107421 | 0.256107421 | loss | 0.71468464 | 0.458577219 | | 37 | 11 |
| cg24254488 | 7.13E−10 | 3.03E−04 | −0.256103896 | 0.256103896 | loss | 0.455007008 | 0.198903112 | | 37 | 7 |
| cg26817877 | 1.19E−09 | 5.07E−04 | −0.256102822 | 0.255102822 | loss | 0.474407492 | 0.218304671 | | 37 | 2 |
| cg14673415 | 7.13E−10 | 3.03E−04 | −0.256052291 | 0.256052291 | loss | 0.679892238 | 0.423839947 | | 37 | 6 |
| cg04512603 | 5.39E−09 | 2.29E−03 | −0.256021321 | 0.256021321 | loss | 0.931154526 | 0.675133206 | | 37 | 7 |
| cg05312960 | 2.56E−09 | 1.09E−03 | −0.256000471 | 0.256000471 | loss | 0.86185653 | 0.605856059 | PIWIL3; PIWIL3; TOP1P2; PIWIL3; | 37 | 22 |
| | | | | | | | | PIWIL3 | | |
| cg11229864 | 1.10E−09 | 4.65E−04 | −0.255999676 | 0.255999676 | loss | 0.767206423 | 0.511206747 | | 37 | 11 |
| cg07555084 | 4.57E−09 | 1.94E−03 | −0.255994918 | 0.255994918 | loss | 0.702897689 | 0.446902771 | | 37 | 6 |
| cg15778434 | 7.13E−10 | 3.03E−04 | −0.255988151 | 0.255988151 | loss | 0.82727494 | 0.571286788 | CACNG8; CACNG6 | 37 | 19 |
| cg16680214 | 2.56E−09 | 1.09E−03 | −0.2559811 | 0.2559811 | loss | 0.4670858 | 0.2111047 | KCNN3; KCNN3 | 37 | 1 |
| cg06093039 | 5.39E−09 | 2.29E−03 | −0.255960761 | 0.255960761 | loss | 0.815665091 | 0.559704329 | GPR133 | 37 | 12 |
| cg18223453 | 7.77E−10 | 3.30E−04 | −0.255957067 | 0.255957067 | loss | 0.728685091 | 0.472728024 | TBX4 | 37 | 17 |
| cg14858616 | 7.77E−10 | 3.30E−04 | −0.255943331 | 0.255943331 | loss | 0.591437649 | 0.335494318 | | 37 | 10 |
| cg03252627 | 7.84E−10 | 3.33E−04 | −0.255921713 | 0.255921713 | loss | 0.776409113 | 0.5204874 | | 37 | 8 |
| cg11196848 | 7.13E−10 | 3.03E−04 | −0.255892982 | 0.255892982 | loss | 0.694861653 | 0.438968671 | FGF20; FGF20 | 37 | 8 |
| cg14488962 | 2.56E−09 | 1.09E−03 | −0.255806737 | 0.255806737 | loss | 0.780199602 | 0.524392865 | NTM | 37 | 11 |
| cg02826525 | 7.13E−10 | 3.03E−04 | −0.255804752 | 0.255804752 | loss | 0.856420911 | 0.600616159 | ARHGEF33; LOC375196 | 37 | 2 |
| cg03451178 | 1.81E−08 | 7.69E−03 | −0.255785663 | 0.255785663 | loss | 0.644566945 | 0.388781282 | METAP1D | 37 | 2 |
| cg20488756 | 1.99E−09 | 8.43E−04 | −0.255747835 | 0.255747835 | loss | 0.847353047 | 0.591605212 | TRIM15 | 37 | 6 |
| cg09879253 | 7.13E−10 | 3.03E−04 | −0.255729475 | 0.255729475 | loss | 0.745145245 | 0.489415771 | | 37 | 17 |
| cg23628563 | 2.69E−08 | 1.14E−02 | −0.255715081 | 0.255715081 | loss | 0.789214434 | 0.533499353 | | 37 | 7 |
| cg24867279 | 9.94E−08 | 4.22E−02 | −0.255705419 | 0.255705419 | loss | 0.524693577 | 0.268988159 | | 37 | 6 |
| cg03890691 | 7.13E−10 | 3.03E−04 | −0.255687522 | 0.255687522 | loss | 0.306565989 | 0.050878467 | DOC2A; DOC2A; DOC2A; DOC2A; | 37 | 16 |
| | | | | | | | | DOC2A; DOC2A; DOC2A | | |
| cg20816104 | 7.13E−10 | 3.03E−04 | −0.255681362 | 0.255681362 | loss | 0.472975957 | 0.217294594 | | 37 | 2 |
| cg21205031 | 7.13E−10 | 3.03E−04 | −0.255669497 | 0.255669497 | loss | 0.625747279 | 0.370077782 | YIPF4 | 37 | 2 |
| cg01258050 | 7.77E−10 | 3.30E−04 | −0.255639458 | 0.255639458 | loss | 0.818341975 | 0.562702518 | KNDC1 | 37 | 10 |
| cg09259772 | 7.13E−10 | 3.03E−04 | −0.255634171 | 0.255634171 | loss | 0.51916073 | 0.263526559 | ARL4A; ARL4A; ARL4A; ARL4A | 37 | 7 |
| cg20675702 | 7.13E−10 | 3.03E−04 | −0.255624023 | 0.255624023 | loss | 0.743205706 | 0.487581682 | | 37 | 10 |
| cg06835125 | 8.47E−10 | 3.60E−04 | −0.255609821 | 0.255609821 | loss | 0.703959257 | 0.448349435 | | 37 | 2 |
| cg00901554 | 7.13E−10 | 3.03E−04 | −0.255591381 | 0.255591381 | loss | 0.832445923 | 0.576854541 | | 37 | 4 |
| cg21593676 | 9.23E−10 | 3.92E−04 | −0.255571019 | 0.255571019 | loss | 0.395550128 | 0.139979109 | KLHL8 | 37 | 4 |
| cg19706795 | 7.13E−10 | 3.03E−04 | −0.255560293 | 0.255560293 | loss | 0.794448323 | 0.538883029 | DFNA5; DFNA5; DFNA5; DFNA5; | 37 | 7 |
| | | | | | | | | DFNA5 | | |
| cg18031326 | 8.47E−10 | 3.60E−04 | −0.255473889 | 0.255473889 | loss | 0.642716772 | 0.387242882 | LRP5 | 37 | 11 |
| cg08818829 | 1.54E−08 | 6.56E−03 | −0.255465515 | 0.255465515 | loss | 0.769336709 | 0.513871194 | DNAH5 | 37 | 5 |
| cg03755115 | 7.13E−10 | 3.03E−04 | −0.255457778 | 0.255457778 | loss | 0.370395857 | 0.114938078 | MYO7A; MYO7A; MYO7A | 37 | 11 |
| cg14640244 | 7.13E−10 | 3.03E−04 | −0.255405123 | 0.255405123 | loss | 0.445676515 | 0.190271392 | LCN10 | 37 | 9 |
| cg01475377 | 1.21E−09 | 5.15E−04 | −0.255386928 | 0.255386928 | loss | 0.837227287 | 0.581840359 | | 37 | 6 |
| cg10115348 | 7.13E−10 | 3.03E−04 | −0.255285573 | 0.255285573 | loss | 0.642864591 | 0.387579018 | ARHGEF10L | 37 | 1 |
| cg18103394 | 1.01E−09 | 4.27E−04 | −0.255264194 | 0.255264194 | loss | 0.794290447 | 0.539026253 | SYN1; SYN1 | 37 | X |
| cg04104776 | 7.13E−10 | 3.03E−04 | −0.255236137 | 0.255236137 | loss | 0.584179096 | 0.328942959 | TRPV3; TRPV3 | 37 | 17 |
| cg16529323 | 1.96E−08 | 8.33E−03 | −0.255216577 | 0.255216577 | loss | 0.718822525 | 0.463605947 | | 37 | 18 |
| cg02151983 | 7.13E−10 | 3.03E−04 | −0.255188733 | 0.255188733 | loss | 0.644760215 | 0.389571482 | | 37 | 3 |
| cg00301396 | 7.13E−10 | 3.03E−04 | −0.25515931 | 0.25515931 | loss | 0.294211477 | 0.039052168 | | 37 | 16 |
| cg21497977 | 7.77E−10 | 3.30E−04 | −0.255154961 | 0.255154961 | loss | 0.433011143 | 0.177856182 | | 37 | 6 |
| cg04617936 | 2.35E−09 | 9.98E−04 | −0.255112619 | 0.255112619 | loss | 0.889433272 | 0.634320653 | | 37 | 2 |
| cg11482879 | 4.21E−09 | 1.79E−03 | −0.255111582 | 0.255111582 | loss | 0.90509047 | 0.649978888 | | 37 | 17 |
| cg16189953 | 5.39E−09 | 2.29E−03 | −0.255103401 | 0.255103401 | loss | 0.624547613 | 0.369444212 | | 37 | 18 |
| cg26923084 | 6.35E−09 | 2.70E−03 | −0.255102398 | 0.255102398 | loss | 0.693420898 | 0.4383185 | NID2 | 37 | 14 |
| cg05211480 | 1.32E−08 | 5.59E−03 | −0.255097612 | 0.255097612 | loss | 0.558594736 | 0.303497124 | | 37 | 14 |
| cg14210678 | 7.13E−10 | 3.03E−04 | −0.255097184 | 0.255097184 | loss | 0.768720608 | 0.513623424 | LMX1B; LMX1B; LMX1B | 37 | 9 |
| cg07311419 | 7.13E−10 | 3.03E−04 | −0.255085203 | 0.255085203 | loss | 0.595610032 | 0.340524829 | | 37 | 11 |
| cg26877596 | 7.77E−10 | 3.30E−04 | −0.255064424 | 0.255064424 | loss | 0.569508847 | 0.314444424 | NUAK1 | 37 | 12 |
| cg14191654 | 7.13E−10 | 3.03E−04 | −0.255057479 | 0.255057479 | loss | 0.313192632 | 0.058135153 | CDH22 | 37 | 20 |
| cg23129573 | 7.13E−10 | 3.03E−04 | −0.254975775 | 0.254975775 | loss | 0.318544264 | 0.063568489 | ELN; ELN; ELN; ELN; ELN; ELN; | 37 | 7 |
| | | | | | | | | ELN; ELN; ELN; ELN; ELN; | | |
| | | | | | | | | ELN; ELN | | |
| cg23424043 | 7.13E−10 | 3.03E−04 | −0.25497454 | 0.25497454 | loss | 0.410811962 | 0.155837422 | ZC3HAV1; ZC3KAV1 | 37 | 7 |
| cg05184311 | 7.13E−10 | 3.03E−04 | −0.254969868 | 0.254969868 | loss | 0.583893926 | 0.328924059 | PKP1; PKP1 | 37 | 1 |
| cg19524572 | 7.13E−10 | 3.03E−04 | −0.254928403 | 0.254928403 | loss | 0.632028268 | 0.377099865 | | 37 | 14 |
| cg05100432 | 7.77E−10 | 3.30E−04 | −0.254911299 | 0.254911299 | loss | 0.863769764 | 0.608858465 | | 37 | 14 |
| cg02677192 | 7.13E−10 | 3.03E−04 | −0.254883189 | 0.254883189 | loss | 0.664576272 | 0.409693082 | PDXK | 37 | 21 |
| cg16672421 | 7.13E−10 | 3.03E−04 | −0.254882015 | 0.254882015 | loss | 0.796291362 | 0.541409347 | CCSAP | 37 | 1 |
| cg20945085 | 7.13E−10 | 3.03E−04 | −0.254859019 | 0.254859019 | loss | 0.320582187 | 0.065723168 | M1AP; M1AP; M1AP; M1AP | 37 | 2 |
| cg18120323 | 4.97E−09 | 2.11E−03 | −0.254785765 | 0.254785765 | loss | 0.496005236 | 0.241219471 | | 37 | 1 |
| cg02602264 | 1.30E−09 | 5.52E−04 | −0.254776895 | 0.254776895 | loss | 0.778052436 | 0.523275541 | CYP2W1 | 37 | 7 |
| cg18680834 | 9.54E−09 | 4.05E−03 | −0.254747871 | 0.254747871 | loss | 0.774394183 | 0.519646312 | ZNF536 | 37 | 19 |
| cg18061197 | 7.77E−10 | 3.30E−04 | −0.254744074 | 0.254744074 | loss | 0.904263868 | 0.649519794 | RGS22; RGS22; RGS22; RGS22 | 37 | 8 |
| cg25037335 | 4.26E−08 | 1.81E−02 | −0.254738261 | 0.254738261 | loss | 0.81183759 | 0.557099329 | ERICH1-AS1; ERICH1-AS1 | 37 | 8 |
| cg16328007 | 2.35E−09 | 9.98E−04 | −0.254725017 | 0.254725017 | loss | 0.487455275 | 0.232730259 | RIMS3 | 37 | 1 |
| cg16532282 | 8.47E−10 | 3.60E−04 | −0.254705171 | 0.254705171 | loss | 0.412801247 | 0.158096076 | | 37 | 1 |
| cg22122321 | 7.13E−10 | 3.03E−04 | −0.254673356 | 0.254673356 | loss | 0.516503874 | 0.261830518 | | 37 | 4 |
| cg02828104 | 7.13E−10 | 3.03E−04 | −0.254630045 | 0.254630045 | loss | 0.542804304 | 0.288174259 | TMEM189; TMEM189; TMEM189- | 37 | 20 |
| | | | | | | | | UBE2V1; TMEM189 | | |
| cg04637454 | 7.13E−10 | 3.03E−04 | −0.254623701 | 0.254623701 | loss | 0.350105774 | 0.095482073 | | 37 | 6 |
| cg15542459 | 7.13E−10 | 3.03E−04 | −0.25458003 | 0.25458003 | loss | 0.564548042 | 0.309968012 | | 37 | 10 |
| cg19180828 | 7.13E−10 | 3.03E−04 | −0.254578001 | 0.254578001 | loss | 0.549282636 | 0.294704635 | RNF207 | 37 | 1 |
| cg05726758 | 7.13E−10 | 3.03E−04 | −0.254567324 | 0.254567324 | loss | 0.798640447 | 0.544073124 | | 37 | 16 |
| cg03270204 | 7.13E−10 | 3.03E−04 | −0.254561959 | 0.254561959 | loss | 0.861404494 | 0.606842535 | DDR1; DDR1; DDR1 | 37 | 6 |
| cg14992524 | 7.13E−10 | 3.03E−04 | −0.254520501 | 0.254520501 | loss | 0.520968813 | 0.266448312 | SOX7 | 37 | 8 |
| cg07138793 | 8.47E−10 | 3.60E−04 | −0.254499016 | 0.254499016 | loss | 0.764627998 | 0.510128982 | OCA2 | 37 | 15 |
| cg13809095 | 1.68E−09 | 7.12E−04 | −0.254427531 | 0.254427531 | loss | 0.762415408 | 0.507987876 | | 37 | 10 |
| cg01963623 | 7.77E−10 | 3.30E−04 | −0.254381885 | 0.254381885 | loss | 0.468711538 | 0.214329653 | USP18 | 37 | 22 |
| cg24479014 | 2.91E−08 | 1.23E−02 | −0.254381556 | 0.254381556 | loss | 0.901257026 | 0.646875471 | WTAPP1 | 37 | 11 |
| cg02854229 | 9.23E−10 | 3.92E−04 | −0.254308498 | 0.254308498 | loss | 0.608322104 | 0.354013606 | | 37 | 14 |
| cg10173814 | 7.77E−10 | 3.30E−04 | −0.254292224 | 0.254292224 | loss | 0.61545613 | 0.361163906 | | 37 | 9 |
| cg17579753 | 7.13E−10 | 3.03E−04 | −0.254289661 | 0.254289661 | loss | 0.390658075 | 0.136368414 | GAS2L2; GAS2L2 | 37 | 17 |
| cg22894896 | 8.47E−10 | 3.60E−04 | −0.254284284 | 0.254284284 | loss | 0.419933513 | 0.165649229 | MIR193A | 37 | 17 |
| cg00660106 | 7.77E−10 | 3.30E−04 | −0.254251286 | 0.254251285 | loss | 0.577044868 | 0.322793582 | KIAA1598 | 37 | 10 |
| cg17867243 | 1.68E−09 | 7.12E−04 | −0.254246 | 0.254246 | loss | 0.765206158 | 0.510960159 | PLA2G4D | 37 | 15 |
| cg20862097 | 7.13E−10 | 3.03E−04 | −0.254234153 | 0.254234153 | loss | 0.637672689 | 0.383438535 | DAAM2; DAAM2 | 37 | 6 |
| cg24332577 | 7.13E−10 | 3.03E−04 | −0.25419559 | 0.25419559 | loss | 0.73975936 | 0.485563771 | SALL4; SALL4 | 37 | 20 |
| cg08602346 | 8.47E−10 | 3.60E−04 | −0.254174729 | 0.254174729 | loss | 0.475626906 | 0.221452176 | TUBGCP3; TUBGCP3; TUBGCP3; | 37 | 13 |
| | | | | | | | | TUBGCP3 | | |
| cg04800569 | 7.13E−10 | 3.03E−04 | −0.254143875 | 0.254143875 | loss | 0.459729681 | 0.205585806 | TMEM190 | 37 | 19 |
| cg00930707 | 7.13E−10 | 3.03E−04 | −0.254102241 | 0.254102241 | loss | 0.844344506 | 0.590242265 | RNPEP | 37 | 1 |
| cg18665594 | 5.84E−08 | 2.48E−02 | −0.254083542 | 0.254083542 | loss | 0.536474425 | 0.282390882 | | 37 | 5 |
| cg27282281 | 5.84E−08 | 2.48E−02 | −0.254064059 | 0.254064059 | loss | 0.787903042 | 0.533838982 | | 37 | 7 |
| cg00907200 | 1.68E−09 | 7.12E−04 | −0.254010695 | 0.254010695 | loss | 0.745192419 | 0.491181724 | ESPNL | 37 | 2 |
| cg08422645 | 5.39E−08 | 2.29E−02 | −0.254005362 | 0.254005362 | loss | 0.519016956 | 0.265011594 | | 37 | 1 |
| cg03332701 | 7.13E−10 | 3.03E−04 | −0.253983866 | 0.253983866 | loss | 0.447143766 | 0.1931599 | | 37 | 15 |
| cg18310895 | 8.47E−10 | 3.60E−04 | −0.253973234 | 0.253973234 | loss | 0.716930334 | 0.4629571 | KCNQ2; KCNQ2; XCNQ2; KCNQ2; | 37 | 20 |
| | | | | | | | | KCNQ2 | | |
| cg21964928 | 3.40E−08 | 1.44E−02 | −0.25395138 | 0.25395138 | loss | 0.519986751 | 0.266035371 | | 37 | 5 |
| cg11797717 | 7.13E−10 | 3.03E−04 | −0.253941689 | 0.253941689 | loss | 0.649686877 | 0.395745188 | PLCD3 | 37 | 17 |
| cg20893919 | 1.99E−09 | 8.43E−04 | −0.253919585 | 0.253919585 | loss | 0.733580085 | 0.4796605 | TRPC3; TRPC3; TRPC3 | 37 | 4 |
| cg11382666 | 7.13E−10 | 3.03E−04 | −0.253918813 | 0.253918813 | loss | 0.848522313 | 0.5946035 | | 37 | 6 |
| cg14354168 | 8.47E−10 | 3.60E−04 | −0.253902044 | 0.253902044 | loss | 0.669162621 | 0.415260576 | | 37 | 6 |
| cg19802390 | 9.23E−10 | 3.92E−04 | −0.253836717 | 0.253836717 | loss | 0.555281834 | 0.301445116 | | 37 | 1 |
| cg09223687 | 7.13E−10 | 3.03E−04 | −0.253809197 | 0.253809197 | loss | 0.892998221 | 0.639189024 | | 37 | 4 |
| cg09784886 | 1.81E−08 | 7.69E−03 | −0.253787181 | 0.253787181 | loss | 0.448462874 | 0.194675692 | KRTAP5-11; KRTAP5-11 | 37 | 11 |
| cg02483204 | 1.03E−08 | 4.39E−03 | −0.253766207 | 0.253766207 | loss | 0.577869513 | 0.324103306 | | 37 | 12 |
| cg19701087 | 8.47E−10 | 3.60E−04 | −0.253726356 | 0.253726356 | loss | 0.548102726 | 0.294376371 | GSTA4 | 37 | 6 |
| cg20516209 | 7.13E−10 | 3.03E−04 | −0.253723829 | 0.253723829 | loss | 0.572516253 | 0.318792424 | EMILIN1 | 37 | 2 |
| cg15283852 | 7.84E−10 | 3.33E−04 | −0.253713734 | 0.253713734 | loss | 0.352638771 | 0.098925038 | USP43; USP43 | 37 | 17 |
| cg03703111 | 7.13E−10 | 3.03E−04 | −0.253713165 | 0.253713165 | loss | 0.540706389 | 0.286993224 | NR2F1; MIR548AO | 37 | 5 |
| cg17156402 | 7.13E−10 | 3.03E−04 | −0.253698915 | 0.253698915 | loss | 0.864666368 | 0.610967453 | FAM131C | 37 | 1 |
| cg10888244 | 7.77E−10 | 3.30E−04 | −0.253687977 | 0.253687977 | loss | 0.773346689 | 0.519658712 | PVR14 | 37 | 1 |
| cg01991625 | 7.13E−10 | 3.03E−04 | −0.253681814 | 0.253681814 | loss | 0.837194543 | 0.583512729 | | 37 | 16 |
| cg22157494 | 7.13E−10 | 3.03E−04 | −0.253656131 | 0.253656131 | loss | 0.727193708 | 0.473537576 | DFFB; DFFB; CEP104; DFFB | 37 | 1 |
| cg20752878 | 7.13E−10 | 3.03E−04 | −0.253618634 | 0.253618634 | loss | 0.728420092 | 0.474801459 | ASGR1; ASGR1 | 37 | 17 |
| cg18964375 | 7.13E−10 | 3.03E−04 | −0.253596872 | 0.253596872 | loss | 0.725527566 | 0.471930694 | | 37 | 1 |
| cg06699564 | 1.68E−09 | 7.12E−04 | −0.253560356 | 0.253560356 | loss | 0.518527568 | 0.264967212 | LOC100288748 | 37 | 8 |
| cg19769301 | 2.35E−09 | 9.98E−04 | 0.253556255 | 0.253556255 | gain | 0.422650945 | 0.6762072 | NBPF23 | 37 | 1 |
| cg27387193 | 7.83E−09 | 3.32E−03 | −0.253546901 | 0.253546901 | loss | 0.518833931 | 0.265287029 | TNXB | 37 | 6 |
| cg24368876 | 3.88E−09 | 1.65E−03 | −0.253542103 | 0.253542103 | loss | 0.740191532 | 0.486649429 | | 37 | 1 |
| cg12689679 | 8.80E−09 | 3.74E−03 | −0.253541662 | 0.253541662 | loss | 0.705797562 | 0.4522559 | LOC283710 | 37 | 15 |
| cg19490561 | 7.13E−10 | 3.03E−04 | −0.253535396 | 0.253535396 | loss | 0.878648302 | 0.625112906 | PDIA6; PDIA6; PDIA6 | 37 | 2 |
| cg02780269 | 7.13E−10 | 3.03E−04 | −0.253525284 | 0.253525284 | loss | 0.481431655 | 0.227906371 | SECTM1 | 37 | 17 |
| cg25782983 | 7.77E−10 | 3.30E−04 | −0.253513754 | 0.253513754 | loss | 0.730467872 | 0.476954118 | TPBGL | 37 | 11 |
| cg17201651 | 7.77E−10 | 3.30E−04 | −0.253486095 | 0.253486095 | loss | 0.554935036 | 0.301448941 | XPNPEP1; XPNPEP1; XPNPEP1 | 37 | 10 |
| cg26600802 | 7.77E−10 | 3.30E−04 | −0.253457418 | 0.253457418 | loss | 0.94617963 | 0.692722212 | TDRD1 | 37 | 10 |
| cg00839584 | 5.01E−08 | 2.13E−02 | −0.2534312 | 0.2534312 | loss | 0.621332306 | 0.367901106 | IL1A | 37 | 2 |
| cg19163067 | 1.30E−09 | 5.52E−04 | −0.253383018 | 0.253383018 | loss | 0.47857024 | 0.225187221 | TFAP2E | 37 | 1 |
| cg10643271 | 1.73E−09 | 7.34E−04 | −0.253328676 | 0.253328676 | loss | 0.446225053 | 0.192896376 | SLC15A4 | 37 | 12 |
| cg23927469 | 7.13E−10 | 3.03E−04 | −0.253328455 | 0.253328455 | loss | 0.536078496 | 0.282750041 | TBC1D14 | 37 | 4 |
| cg13749379 | 7.84E−10 | 3.33E−04 | −0.253321207 | 0.253321207 | loss | 0.319439192 | 0.066117985 | SOST | 37 | 17 |
| cg21844144 | 1.39E−08 | 5.90E−03 | −0.253269768 | 0.253269768 | loss | 0.707614521 | 0.454344753 | LOC338694 | 37 | 11 |
| cg12097325 | 1.67E−08 | 7.10E−03 | −0.253267416 | 0.253267416 | loss | 0.748129092 | 0.494861676 | LOC254099 | 37 | 1 |
| cg21811204 | 7.13E−10 | 3.03E−04 | −0.25324366 | 0.25324366 | loss | 0.369883998 | 0.116640338 | SHROOM1; SHROOM1 | 37 | 5 |
| cg05866737 | 5.39E−09 | 2.29E−03 | −0.253206689 | 0.253206689 | loss | 0.73154273 | 0.478336041 | KRTAP5-5; MOB2 | 37 | 11 |
| cg14463912 | 7.13E−10 | 3.03E−04 | −0.253204031 | 0.253204031 | loss | 0.679230366 | 0.426026335 | PDZD7; PDZD7; SFXN3 | 37 | 10 |
| cg18197594 | 3.28E−09 | 1.39E−03 | −0.253197919 | 0.253197919 | loss | 0.634401908 | 0.381203988 | IDE | 37 | 10 |
| cg22047901 | 7.13E−10 | 3.03E−04 | −0.253174368 | 0.253174368 | loss | 0.577859515 | 0.324685147 | MIR96; MIR183 | 37 | 7 |
| cg04839131 | 7.13E−10 | 3.03E−04 | −0.253162959 | 0.253162959 | loss | 0.41043943 | 0.157276471 | KCNH2; KCNH2 | 37 | 7 |
| cg19843891 | 7.13E−10 | 3.03E−04 | −0.253111605 | 0.253111605 | loss | 0.338031289 | 0.084919684 | | 37 | 8 |
| cg13630131 | 7.13E−10 | 3.03E−04 | −0.253107577 | 0.253107577 | loss | 0.692720883 | 0.439613306 | | 37 | 7 |
| cg02174092 | 4.97E−09 | 2.11E−03 | −0.253104241 | 0.253104241 | loss | 0.326060328 | 0.072956088 | | 37 | 10 |
| cg02664787 | 2.56E−09 | 1.09E−03 | −0.253018911 | 0.253018911 | loss | 0.610062031 | 0.357043171 | OPCML | 37 | 11 |
| cg04872541 | 5.85E−09 | 2.49E−03 | −0.253017138 | 0.253017138 | loss | 0.678258921 | 0.425241782 | PKD2L1; PKD2L1; PKD2L1; | 37 | 10 |
| | | | | | | | | PKD2L1 | | |
| cg06814287 | 7.13E−10 | 3.03E−04 | −0.253006503 | 0.253006503 | loss | 0.928586979 | 0.675580476 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFLS | | |
| cg13504055 | 1.54E−09 | 6.54E−04 | −0.252898967 | 0.252898967 | loss | 0.772572296 | 0.519673329 | | 37 | 2 |
| cg22369590 | 7.13E−10 | 3.03E−04 | −0.252892501 | 0.252892501 | loss | 0.78234593 | 0.529453429 | HAPLN2 | 37 | 1 |
| cg13904562 | 9.23E−10 | 3.92E−04 | −0.252753351 | 0.252753351 | loss | 0.574804857 | 0.322051506 | | 37 | 12 |
| cg08021797 | 1.73E−09 | 7.34E−04 | −0.252662763 | 0.252662763 | loss | 0.503173613 | 0.25051085 | C16orf74 | 37 | 16 |
| cg27612019 | 7.13E−10 | 3.03E−04 | −0.252638054 | 0.252638054 | loss | 0.68212103 | 0.429482976 | SLC25A18 | 37 | 22 |
| cg12070285 | 7.13E−10 | 3.03E−04 | −0.252623988 | 0.252623988 | loss | 0.894369717 | 0.641745729 | KHDC3L | 37 | 6 |
| cg01783554 | 7.13E−10 | 3.03E−04 | −0.252617312 | 0.252617312 | loss | 0.866714836 | 0.614097524 | | 37 | 8 |
| cg23144063 | 7.13E−10 | 3.03E−04 | −0.252597226 | 0.252597226 | loss | 0.420401179 | 0.167803953 | FAM107A; FAM107A | 37 | 3 |
| cg10336790 | 1.96E−08 | 8.33E−03 | −0.252570921 | 0.252570921 | loss | 0.573555815 | 0.320984894 | GPR85; GPR85; GPR85; GPR85 | 37 | 7 |
| cg11789681 | 7.13E−10 | 3.03E−04 | −0.252555489 | 0.252555489 | loss | 0.626510136 | 0.373954647 | | 37 | 15 |
| cg20929609 | 7.13E−10 | 3.03E−04 | −0.25254656 | 0.25254656 | loss | 0.629068119 | 0.376521559 | RLF | 37 | 1 |
| cg11194698 | 1.83E−09 | 7.75E−04 | −0.252534939 | 0.252534939 | loss | 0.788736515 | 0.536201576 | GCK; GCK; GCK | 37 | 7 |
| cg17168157 | 7.13E−10 | 3.03E−04 | −0.252532074 | 0.252532074 | loss | 0.560938074 | 0.308406 | RGS22; RGS22; RGS22 | 37 | 8 |
| cg24919768 | 9.23E−10 | 3.92E−04 | −0.25252701 | 0.25252701 | loss | 0.455078926 | 0.203551916 | MALL | 37 | 2 |
| cg07755203 | 1.83E−09 | 7.75E−04 | −0.252521446 | 0.252521446 | loss | 0.70351747 | 0.450996024 | | 37 | 2 |
| cg11950360 | 7.13E−10 | 3.03E−04 | −0.252518435 | 0.252518435 | loss | 0.5655325 | 0.313014065 | CELF4; CELF4; CELF4; CELF4 | 37 | 18 |
| cg03184588 | 5.41E−08 | 2.30E−02 | −0.252495828 | 0.252495828 | loss | 0.591732792 | 0.339236965 | FAM60A; FAM60A; FAM60A | 37 | 12 |
| cg02198562 | 3.88E−09 | 1.65E−03 | −0.252481152 | 0.252481152 | loss | 0.863053934 | 0.610572782 | | 37 | 11 |
| cg13092405 | 5.41E−08 | 2.30E−02 | −0.252480301 | 0.252480301 | loss | 0.468437768 | 0.215957466 | | 37 | 1 |
| cg03015498 | 1.22E−08 | 5.16E−03 | −0.252429811 | 0.252429811 | loss | 0.732862458 | 0.480432647 | | 37 | 10 |
| cg08942800 | 7.48E−09 | 3.18E−03 | −0.252411801 | 0.252411801 | loss | 0.799317025 | 0.546905224 | CRISP2; CRISP2; CRISP2; CRISP2; | 37 | 6 |
| | | | | | | | | CRISP2; CRISP2; CRISP2; | | |
| | | | | | | | | CRISP2; CRISP2; CRISP2; | | |
| | | | | | | | | CRISP2; CRISP2 | | |
| cg03724964 | 7.13E−10 | 3.03E−04 | −0.252382086 | 0.252382086 | loss | 0.495845498 | 0.243463412 | FBXO17; F8XO17 | 37 | 19 |
| cg14716095 | 7.13E−10 | 3.03E−04 | −0.25236452 | 0.25236452 | loss | 0.534691579 | 0.282327059 | | 37 | 13 |
| cg12552293 | 7.13E−10 | 3.03E−04 | −0.252353794 | 0.252353794 | loss | 0.556255153 | 0.303901359 | C17orf98; C17orf98 | 37 | 17 |
| cg25696972 | 1.01E−09 | 4.27E−04 | −0.252352833 | 0.252352833 | loss | 0.841214462 | 0.588861629 | | 37 | 6 |
| cg03211420 | 9.54E−09 | 4.05E−03 | −0.252350441 | 0.252350441 | loss | 0.603866117 | 0.351515676 | | 37 | 6 |
| cg11433098 | 2.35E−09 | 9.98E−04 | −0.252320634 | 0.252320634 | loss | 0.432533675 | 0.180213041 | | 37 | 15 |
| cg04254769 | 5.85E−09 | 2.49E−03 | −0.252315988 | 0.252315988 | loss | 0.869331817 | 0.617015829 | | 37 | 5 |
| cg14266410 | 9.23E−10 | 3.92E−04 | −0.252307843 | 0.252307843 | loss | 0.89481386 | 0.642506018 | | 37 | 14 |
| cg26286839 | 3.02E−09 | 1.28E−03 | −0.252280628 | 0.252280628 | loss | 0.685301428 | 0.4330208 | THBS4 | 37 | 5 |
| cg16919708 | 4.29E−08 | 1.82E−02 | −0.252279871 | 0.252279871 | loss | 0.584276577 | 0.331996706 | OPCML | 37 | 11 |
| cg20636352 | 1.83E−09 | 7.75E−04 | −0.252267463 | 0.252267463 | loss | 0.602611034 | 0.350343571 | | 37 | 2 |
| cg13906860 | 5.39E−09 | 2.29E−03 | −0.252261761 | 0.252261761 | loss | 0.388936915 | 0.136675154 | | 37 | 11 |
| cg04249191 | 1.54E−09 | 6.54E−04 | −0.252257659 | 0.252257659 | loss | 0.812171836 | 0.559914176 | | 37 | 1 |
| cg08635489 | 7.13E−10 | 3.03E−04 | −0.252257048 | 0.252257048 | loss | 0.871462725 | 0.619205676 | | 37 | 1 |
| cg12828018 | 7.77E−10 | 3.30E−04 | −0.252225369 | 0.252225369 | loss | 0.737719834 | 0.485494465 | PATL1 | 37 | 11 |
| cg20673819 | 2.91E−08 | 1.23E−02 | −0.252222774 | 0.252222774 | loss | 0.945069009 | 0.692846235 | LINC00473; LINC00473 | 37 | 6 |
| cg16610412 | 8.47E−10 | 3.60E−04 | −0.252198646 | 0.252198646 | loss | 0.917170834 | 0.664972188 | FSTL4 | 37 | 5 |
| cg14825555 | 7.13E−10 | 3.03E−04 | −0.252189626 | 0.252189626 | loss | 0.406092396 | 0.153902771 | SFN | 37 | 1 |
| cg20467658 | 1.67E−08 | 7.10E−03 | −0.252183295 | 0.252183295 | loss | 0.645791736 | 0.393608441 | LINC00925 | 37 | 15 |
| cg05930207 | 1.30E−09 | 5.52E−04 | −0.252168619 | 0.252168619 | loss | 0.766795608 | 0.514626988 | GJD3 | 37 | 17 |
| cg07880854 | 7.13E−10 | 3.03E−04 | −0.252147569 | 0.252147569 | loss | 0.500395051 | 0.248247482 | FBLN7; FBLN7 | 37 | 2 |
| cg15849368 | 7.13E−10 | 3.03E−04 | −0.252114173 | 0.252114173 | loss | 0.570119591 | 0.318005418 | | 37 | 17 |
| cg16761096 | 1.19E−09 | 5.07E−04 | −0.252108903 | 0.252108903 | loss | 0.787872915 | 0.535764012 | COBL | 37 | 7 |
| cg24716558 | 7.13E−10 | 3.03E−04 | −0.252105055 | 0.252105055 | loss | 0.537623208 | 0.285518153 | USH1G; USH1G | 37 | 17 |
| cg24179445 | 1.10E−09 | 4.65E−04 | −0.252078963 | 0.252078963 | loss | 0.550927792 | 0.298848829 | | 37 | 10 |
| cg18105179 | 7.77E−10 | 3.30E−04 | −0.252064866 | 0.252054866 | loss | 0.901870425 | 0.649805559 | GAL3ST2 | 37 | 2 |
| cg20714328 | 7.13E−10 | 3.03E−04 | −0.252048316 | 0.252048316 | loss | 0.754790134 | 0.502741818 | THBS4 | 37 | 5 |
| cg13269964 | 7.13E−10 | 3.03E−04 | −0.252004597 | 0.252004597 | loss | 0.816824485 | 0.564819888 | CD300LG; CD300LG; CD300LG; | 37 | 17 |
| | | | | | | | | CD300LG | | |
| cg22233974 | 7.13E−10 | 3.03E−04 | −0.25197683 | 0.25197683 | loss | 0.498438225 | 0.246461394 | | 37 | 1 |
| cg19821361 | 3.57E−09 | 1.52E−03 | −0.251952645 | 0.251962645 | loss | 0.492964719 | 0.241002074 | TSPEAR; TSPEAR; KRTAP10- | 37 | 21 |
| | | | | | | | | 6; KRTAP10-6 | | |
| cg23009419 | 7.13E−10 | 3.03E−04 | −0.251950649 | 0.251950649 | loss | 0.495350302 | 0.243399653 | TDGF1; TDGF1 | 37 | 3 |
| cg27551856 | 1.22E−08 | 5.16E−03 | −0.251924786 | 0.251924786 | loss | 0.898234157 | 0.646309371 | | 37 | 16 |
| cg10093648 | 7.13E−10 | 3.03E−04 | −0.251906169 | 0.251906169 | loss | 0.49486334 | 0.242957171 | ASPHD1 | 37 | 16 |
| cg12875241 | 1.43E−08 | 6.06E−03 | −0.251885289 | 0.251885289 | loss | 0.578801364 | 0.326916075 | GPR143; GPR143 | 37 | X |
| cg00720629 | 7.13E−10 | 3.03E−04 | −0.251871486 | 0.251871486 | loss | 0.789352245 | 0.537480759 | TBX1; TBX1; TBX1 | 37 | 22 |
| cg05756954 | 4.57E−09 | 1.94E−03 | −0.251860872 | 0.251860872 | loss | 0.394764258 | 0.142903386 | | 37 | X |
| cg07173688 | 7.13E−10 | 3.03E−04 | −0.2518606 | 0.2518606 | loss | 0.601685794 | 0.349825194 | | 37 | 2 |
| cg16343401 | 7.13E−10 | 3.03E−04 | −0.251759562 | 0.251759562 | loss | 0.339184215 | 0.087424654 | GFAP; GFAP; GFAP | 37 | 17 |
| cg01675158 | 7.13E−10 | 3.03E−04 | −0.251721743 | 0.251721743 | loss | 0.762301543 | 0.5105798 | CCDC85C | 37 | 14 |
| cg11654325 | 7.13E−10 | 3.03E−04 | −0.251675763 | 0.251675763 | loss | 0.570980187 | 0.319304424 | CXXC11 | 37 | 2 |
| cg14814062 | 7.13E−10 | 3.03E−04 | −0.251669031 | 0.251669031 | loss | 0.525458066 | 0.273819035 | TTYH1; TTYH1; TTYH1 | 37 | 19 |
| cg22728830 | 4.21E−09 | 1.79E−03 | −0.251575295 | 0.251575296 | loss | 0.576236513 | 0.324661218 | | 37 | 11 |
| cg04284140 | 9.23E−10 | 3.92E−04 | −0.25155554 | 0.25155554 | loss | 0.55452284 | 0.3029673 | POLD2; POLD2; POLD2 | 37 | 7 |
| cg26250086 | 5.01E−08 | 2.13E−02 | −0.251544269 | 0.251544269 | loss | 0.664581581 | 0.413037312 | | 37 | 1 |
| cg12613020 | 4.67E−09 | 1.98E−03 | −0.251499827 | 0.251499827 | loss | 0.4132936 | 0.161793773 | | 37 | 5 |
| cg03540622 | 7.77E−10 | 3.30E−04 | −0.251476445 | 0.251476445 | loss | 0.832180815 | 0.580704371 | ZBTB47 | 37 | 3 |
| cg10621809 | 7.13E−10 | 3.03E−04 | −0.251471686 | 0.251471686 | loss | 0.596741357 | 0.345269671 | | 37 | 12 |
| cg23681730 | 2.30E−08 | 9.75E−03 | −0.251461537 | 0.251461537 | loss | 0.526533849 | 0.275072312 | TRIM44 | 37 | 11 |
| cg16171484 | 7.13E−10 | 3.03E−04 | −0.251447277 | 0.251447277 | loss | 0.654674277 | 0.403227 | SGPP2 | 37 | 2 |
| cg23648082 | 7.13E−10 | 3.03E−04 | −0.251405826 | 0.251405826 | loss | 0.690770349 | 0.439364524 | TMC2 | 37 | 20 |
| cg01346718 | 7.13E−10 | 3.03E−04 | −0.251394887 | 0.251394887 | loss | 0.65430734 | 0.402912453 | CSNK1E; CSNK1E | 37 | 22 |
| cg08770961 | 7.13E−10 | 3.03E−04 | −0.251393376 | 0.251393376 | loss | 0.717185817 | 0.465792441 | RRAD; RRAD | 37 | 16 |
| cg11794215 | 7.13E−10 | 3.03E−04 | −0.251293439 | 0.251293439 | loss | 0.609063151 | 0.357769712 | | 37 | 17 |
| cg08370178 | 1.01E−09 | 4.27E−04 | −0.251287235 | 0.251257235 | loss | 0.854083358 | 0.602796124 | | 37 | 5 |
| cg16481280 | 7.13E−10 | 3.03E−04 | −0.251284441 | 0.251284441 | loss | 0.929759517 | 0.678475376 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg18129759 | 7.77E−10 | 3.30E−04 | −0.251255424 | 0.251255424 | loss | 0.838686736 | 0.587431312 | GPR37L1 | 37 | 1 |
| cg18537730 | 7.13E−10 | 3.03E−04 | −0.251247732 | 0.251247732 | loss | 0.301628455 | 0.050380722 | IZUMO1; IZUMO1 | 37 | 19 |
| cg07504598 | 7.77E−10 | 3.30E−04 | −0.251245409 | 0.251245409 | loss | 0.553744626 | 0.302499218 | DFNA5; DFNA5; DFNA5; DFNA5; | 37 | 7 |
| | | | | | | | | DFNA5 | | |
| cg16537292 | 3.02E−09 | 1.28E−03 | −0.251231191 | 0.251231191 | loss | 0.799136026 | 0.547904835 | ERICH1-AS1; ERICH1-AS1 | 37 | 8 |
| cg14585415 | 7.13E−10 | 3.03E−04 | −0.251223303 | 0.251223303 | loss | 0.759725132 | 0.508501829 | PDZD7; PDZD7 | 37 | 10 |
| cg05130518 | 8.47E−10 | 3.60E−04 | −0.251180118 | 0.251180118 | loss | 0.748902753 | 0.497722635 | IL17D | 37 | 13 |
| cg00057231 | 7.13E−10 | 3.03E−04 | −0.251148888 | 0.251148888 | loss | 0.658729906 | 0.407581018 | COL7A1 | 37 | 3 |
| cg05293216 | 1.67E−08 | 7.10E−03 | −0.251141269 | 0.251141269 | loss | 0.80117744 | 0.550036171 | FANCG | 37 | 9 |
| cg02012483 | 9.23E−10 | 3.92E−04 | −0.25112621 | 0.25112621 | loss | 0.946550034 | 0.695423824 | TRIM15 | 37 | 6 |
| cg03499943 | 5.39E−09 | 2.29E−03 | −0.251109549 | 0.251109549 | loss | 0.957578802 | 0.706469253 | LHPP; LHPP | 37 | 10 |
| cg22738655 | 7.77E−10 | 3.30E−04 | −0.251099804 | 0.251099804 | loss | 0.686956998 | 0.435857194 | UCMA; UCMA | 37 | 10 |
| cg12899423 | 7.13E−10 | 3.03E−04 | −0.251083863 | 0.251083863 | loss | 0.588362704 | 0.337278841 | ALX4 | 37 | 11 |
| cg04307107 | 1.30E−09 | 5.52E−04 | −0.251007804 | 0.251007804 | loss | 0.601287751 | 0.350279947 | NFATC2; NFATC2; NFATC2; | 37 | 20 |
| | | | | | | | | NFATC2 | | |
| cg18410945 | 7.13E−10 | 3.03E−04 | −0.251001498 | 0.251001498 | loss | 0.57371434 | 0.322712841 | | 37 | 7 |
| cg07262335 | 7.13E−10 | 3.03E−04 | −0.251001216 | 0.251001216 | loss | 0.681000151 | 0.429998935 | TCF7L1 | 37 | 2 |
| cg17463698 | 8.47E−10 | 3.60E−04 | −0.250998191 | 0.250998191 | loss | 0.839210421 | 0.588212229 | WHAMM | 37 | 15 |
| cg15731001 | 6.30E−08 | 2.68E−02 | −0.250981377 | 0.250981377 | loss | 0.722323877 | 0.4713425 | SLC6A8; SLC6A8; SLC6A8 | 37 | X |
| cg17862447 | 1.83E−09 | 7.75E−04 | −0.250949381 | 0.250949381 | loss | 0.86606274 | 0.615113359 | KLHL29 | 37 | 2 |
| cg13689996 | 8.55E−08 | 3.63E−02 | −0.250881532 | 0.250881532 | loss | 0.750983238 | 0.500101706 | LINC00469 | 37 | 17 |
| cg12197752 | 2.78E−09 | 1.18E−03 | −0.250868146 | 0.250868146 | loss | 0.636115081 | 0.385246935 | | 37 | 5 |
| cg00642615 | 7.13E−10 | 3.03E−04 | −0.25084515 | 0.25084515 | loss | 0.530593679 | 0.279748529 | PAQR5 | 37 | 15 |
| cg08122232 | 7.13E−10 | 3.03E−04 | −0.250820636 | 0.250820636 | loss | 0.59580333 | 0.344982694 | EXOC7; EXOC7; EXOC7; EXOC7; | 37 | 17 |
| | | | | | | | | EXOC7; EXOC7; EXOC7 | | |
| cg14089267 | 1.54E−09 | 6.54E−04 | −0.250779179 | 0.250779179 | loss | 0.389924134 | 0.139144955 | | 37 | 13 |
| cg02304584 | 3.67E−08 | 1.56E−02 | −0.250744461 | 0.250744461 | loss | 0.579035696 | 0.328291235 | | 37 | 6 |
| cg13269555 | 1.01E−09 | 4.27E−04 | −0.250711431 | 0.250711431 | loss | 0.470298066 | 0.219586635 | KAZALD1 | 37 | 10 |
| cg02275035 | 7.77E−10 | 3.30E−04 | −0.2507047 | 0.2507047 | loss | 0.734121542 | 0.483416841 | | 37 | 14 |
| cg23075506 | 1.32E−09 | 5.62E−04 | −0.250695998 | 0.250695998 | loss | 0.891393715 | 0.640697718 | | 37 | 1 |
| cg03871267 | 7.77E−10 | 3.30E−04 | −0.250636061 | 0.250636061 | loss | 0.697275285 | 0.446639224 | | 37 | 5 |
| cg25201101 | 7.84E−10 | 3.33E−04 | −0.25061881 | 0.25061881 | loss | 0.762565387 | 0.511946576 | RAN | 37 | 12 |
| cg01881287 | 7.13E−10 | 3.03E−04 | −0.250609707 | 0.250609707 | loss | 0.792882466 | 0.542272759 | | 37 | 7 |
| cg27416261 | 7.13E−10 | 3.03E−04 | −0.250576754 | 0.250576754 | loss | 0.470296783 | 0.219720029 | ATP8B5P; ATP8B5P | 37 | 9 |
| cg24415728 | 1.68E−09 | 7.12E−04 | −0.250572981 | 0.250572981 | loss | 0.69080004 | 0.440227059 | | 37 | 8 |
| cg10831285 | 8.47E−10 | 3.60E−04 | −0.250563021 | 0.250563021 | loss | 0.316889536 | 0.066326515 | B3GNT3 | 37 | 19 |
| cg12078872 | 7.13E−10 | 3.03E−04 | −0.250560626 | 0.250560626 | loss | 0.869573549 | 0.619012924 | DDO; DDO | 37 | 6 |
| cg26750002 | 7.13E−10 | 3.03E−04 | −0.250550941 | 0.250550941 | loss | 0.683449406 | 0.432898465 | RASIP1; IZUMO1 | 37 | 19 |
| cg08947894 | 7.13E−10 | 3.03E−04 | −0.250534707 | 0.250534707 | loss | 0.696238583 | 0.445703876 | | 37 | 1 |
| cg21566642 | 7.77E−10 | 3.30E−04 | −0.250526064 | 0.250526064 | loss | 0.501062494 | 0.250536431 | | 37 | 2 |
| cg16166968 | 7.13E−10 | 3.03E−04 | −0.250490996 | 0.250490996 | loss | 0.727330102 | 0.476839106 | ADAM30; ADAM30 | 37 | 1 |
| cg00583861 | 7.13E−10 | 3.03E−04 | −0.250482796 | 0.250482796 | loss | 0.962742443 | 0.712259647 | | 37 | 1 |
| cg19733042 | 7.13E−10 | 3.03E−04 | −0.250464948 | 0.250464948 | loss | 0.740223289 | 0.489758341 | NBPF8; NBPF8; NBPF8 | 37 | 1 |
| cg20743080 | 7.13E−10 | 3.03E−04 | −0.250436661 | 0.250436661 | loss | 0.617618732 | 0.367182071 | | 37 | 4 |
| cg12020444 | 7.13E−10 | 3.03E−04 | −0.250404964 | 0.250404964 | loss | 0.870226123 | 0.619821159 | PIWIL1; PIWIL1 | 37 | 12 |
| cg12209693 | 7.13E−10 | 3.03E−04 | −0.250355065 | 0.250355065 | loss | 0.372929142 | 0.122574076 | GTF2IRD1; GTF2IRD1; GTF2IRD1 | 37 | 7 |
| cg25212270 | 7.77E−10 | 3.30E−04 | −0.250300391 | 0.250300391 | loss | 0.681310038 | 0.431009647 | | 37 | 15 |
| cg12024822 | 9.23E−10 | 3.92E−04 | −0.250296768 | 0.250296768 | loss | 0.475148862 | 0.224852094 | | 37 | 6 |
| cg06406026 | 6.89E−09 | 2.93E−03 | −0.250273756 | 0.250273756 | loss | 0.808348826 | 0.558075071 | | 37 | 10 |
| cg22105158 | 7.13E−10 | 3.03E−04 | −0.250262943 | 0.250262943 | loss | 0.662620855 | 0.412357912 | C19orf77; C19orf77 | 37 | 19 |
| cg05090351 | 1.01E−09 | 4.27E−04 | −0.250249002 | 0.250249002 | loss | 0.607113038 | 0.356864035 | | 37 | 10 |
| cg23553912 | 8.47E−10 | 3.60E−04 | −0.250241506 | 0.250241506 | loss | 0.391116153 | 0.140874646 | SCHIP1; SCHIP1; SCHIP1; IQCJ- | 37 | 3 |
| | | | | | | | | SCHIP1; IQCJ- | | |
| | | | | | | | | SCHIP1; SCHIP1 | | |
| cg16652937 | 7.77E−10 | 3.30E−04 | −0.250240784 | 0.250240784 | loss | 0.795963002 | 0.545722218 | UBE2E2 | 37 | 3 |
| cg19849544 | 7.77E−10 | 3.30E−04 | −0.250239067 | 0.250239067 | loss | 0.696817379 | 0.446578312 | | 37 | 8 |
| cg10430077 | 7.13E−10 | 3.03E−04 | −0.250216885 | 0.250216885 | loss | 0.438121943 | 0.187905059 | | 37 | 5 |
| cg15365218 | 7.77E−10 | 3.30E−04 | −0.250212377 | 0.250212377 | loss | 0.744193242 | 0.493980865 | | 37 | 11 |
| cg24406754 | 7.13E−10 | 3.03E−04 | −0.250203688 | 0.250203688 | loss | 0.931784953 | 0.681581265 | C12orf56; C12orf56 | 37 | 12 |
| cg12160664 | 7.13E−10 | 3.03E−04 | −0.250194685 | 0.250194685 | loss | 0.510415821 | 0.260221135 | OLFML3; OLFML3; OLFML3 | 37 | 1 |
| cg15442434 | 7.13E−10 | 3.03E−04 | −0.250192856 | 0.250192856 | loss | 0.625646779 | 0.375453924 | | 37 | 6 |
| cg21378403 | 7.13E−10 | 3.03E−04 | −0.250178253 | 0.250178253 | loss | 0.465054089 | 0.214875835 | PDZD7; PDZD7 | 37 | 10 |
| cg24369989 | 7.13E−10 | 3.03E−04 | −0.250168986 | 0.250168986 | loss | 0.537479451 | 0.287310465 | CHRNB4; CHRNB4 | 37 | 15 |
| cg08638044 | 7.13E−10 | 3.03E−04 | −0.250157741 | 0.250157741 | loss | 0.693994353 | 0.443836612 | | 37 | 15 |
| cg25013303 | 7.77E−10 | 3.30E−04 | −0.250150107 | 0.250150107 | loss | 0.82102826 | 0.570878153 | | 37 | 1 |
| cg17099016 | 7.13E−10 | 3.03E−04 | −0.250126573 | 0.250126573 | loss | 0.419439326 | 0.169312753 | OTOG | 37 | 11 |
| cg27031448 | 9.23E−10 | 3.92E−04 | −0.250122438 | 0.250122438 | loss | 0.458547226 | 0.208424788 | GTF3A | 37 | 13 |
| cg22591480 | 8.47E−10 | 3.60E−04 | −0.25011697 | 0.25011697 | loss | 0.852841458 | 0.632724488 | SLC16A12 | 37 | 10 |
| cg18212064 | 7.13E−10 | 3.03E−04 | −0.250105929 | 0.250105929 | loss | 0.757022223 | 0.506916294 | | 37 | 8 |
| cg04975966 | 7.13E−10 | 3.03E−04 | −0.250094077 | 0.250094077 | loss | 0.870101183 | 0.620007106 | ALX3 | 37 | 1 |
| cg03303857 | 7.77E−10 | 3.30E−04 | −0.250093901 | 0.250093901 | loss | 0.498089213 | 0.247995312 | GRIK4; GRIK4; GRIK4 | 37 | 11 |
| cg23484899 | 7.13E−10 | 3.03E−04 | −0.250080793 | 0.250080793 | loss | 0.715057581 | 0.464976788 | | 37 | 1 |
| cg20996314 | 7.13E−10 | 3.03E−04 | −0.25007375 | 0.25007375 | loss | 0.670720085 | 0.420646335 | HR; HR | 37 | 8 |
| cg27343801 | 7.13E−10 | 3.03E−04 | −0.250052255 | 0.250052255 | loss | 0.874087938 | 0.624035682 | NOVA2 | 37 | 19 |
| cg17623793 | 1.01E−09 | 4.27E−04 | −0.250019514 | 0.250019514 | loss | 0.891580591 | 0.641561076 | | 37 | 5 |
| cg26349358 | 7.13E−10 | 3.03E−04 | −0.249995844 | 0.249995844 | loss | 0.595710474 | 0.345714629 | | 37 | 2 |
| cg10256477 | 7.13E−10 | 3.03E−04 | −0.249931452 | 0.249931452 | loss | 0.676865223 | 0.426933771 | KCNH3 | 37 | 12 |
| cg08087655 | 2.35E−09 | 9.98E−04 | −0.249870149 | 0.249870149 | loss | 0.822722755 | 0.572852606 | MIR100HG | 37 | 11 |
| cg20005057 | 7.13E−10 | 3.03E−04 | −0.249846364 | 0.249846364 | loss | 0.561373058 | 0.311526694 | GABARAPL1 | 37 | 12 |
| cg26644395 | 2.30E−08 | 9.75E−03 | −0.24983687 | 0.24983687 | loss | 0.751879753 | 0.502042882 | UCN3 | 37 | 10 |
| cg17506621 | 9.54E−09 | 4.05E−03 | −0.249756256 | 0.249756256 | loss | 0.564553862 | 0.314797606 | | 37 | 6 |
| cg01795776 | 7.13E−10 | 3.03E−04 | −0.249698128 | 0.249698128 | loss | 0.833031834 | 0.583333706 | | 37 | 1 |
| cg27413508 | 7.77E−10 | 3.30E−04 | −0.249679705 | 0.249679705 | loss | 0.613859823 | 0.364180118 | COX4I2 | 37 | 20 |
| cg00060374 | 3.40E−08 | 1.44E−02 | −0.249669109 | 0.249669109 | loss | 0.847588221 | 0.597919112 | ANKRD65; ANKRD65; ANKRD65 | 37 | 1 |
| cg07839926 | 1.02E−09 | 4.33E−04 | −0.249571827 | 0.249571827 | loss | 0.799320798 | 0.549748971 | | 37 | 10 |
| cg17570276 | 7.84E−10 | 3.33E−04 | −0.249567635 | 0.249567635 | loss | 0.799068535 | 0.5495009 | EIF2AK1; EIF2AK1 | 37 | 7 |
| cg05045817 | 7.77E−10 | 3.30E−04 | −0.249557923 | 0.249557923 | loss | 0.522193558 | 0.272635635 | | 37 | 1 |
| cg07480640 | 4.57E−09 | 1.94E−03 | −0.249537953 | 0.249537953 | loss | 0.675290594 | 0.425752641 | PRICKLE2; PRICKLE2 | 37 | 3 |
| cg20432350 | 7.77E−10 | 3.30E−04 | −0.249535423 | 0.249535423 | loss | 0.483401911 | 0.233866488 | KCND3; KCND3 | 37 | 1 |
| cg03025830 | 7.34E−08 | 3.12E−02 | −0.249529944 | 0.249529944 | loss | 0.423335077 | 0.173805134 | FGF17 | 37 | 8 |
| cg17571266 | 2.35E−09 | 9.98E−04 | −0.249483808 | 0.249483808 | loss | 0.642571902 | 0.393088094 | ZNF385A;ZNF385A; ZNFF385A | 37 | 12 |
| cg21729798 | 7.34E−08 | 3.12E−02 | −0.249482536 | 0.249482536 | loss | 0.803424494 | 0.553941959 | DCDC2; DCDC2; KAAG1 | 37 | 6 |
| cg04349021 | 8.47E−10 | 3.60E−04 | −0.24946697 | 0.24946697 | loss | 0.553748006 | 0.304281035 | ANKRD33; ANKRD33 | 37 | 12 |
| cg25943845 | 8.47E−10 | 3.60E−04 | −0.2494623 | 0.2494623 | loss | 0.804478258 | 0.555015959 | GDF2; GDF2 | 37 | 10 |
| cg06159340 | 7.13E−10 | 3.03E−04 | −0.249459637 | 0.249459637 | loss | 0.628718902 | 0.379259265 | CTF1; CTF1 | 37 | 16 |
| cg25745746 | 7.13E−10 | 3.03E−04 | −0.249451813 | 0.249451813 | loss | 0.50384976 | 0.254397947 | VWA5B1 | 37 | 1 |
| cg17908602 | 8.47E−10 | 3.60E−04 | −0.249436008 | 0.249436008 | loss | 0.886554885 | 0.637118876 | | 37 | 1 |
| cg07102320 | 1.32E−08 | 5.59E−03 | −0.249428404 | 0.249428404 | loss | 0.742914151 | 0.493485747 | | 37 | 11 |
| cg08634357 | 7.77E−10 | 3.30E−04 | −0.249413754 | 0.249413754 | loss | 0.660245013 | 0.410831259 | RRAD; RRAD | 37 | 16 |
| cg07925738 | 1.54E−09 | 6.54E−04 | −0.249413089 | 0.249413089 | loss | 0.87436123 | 0.624948141 | | 37 | 1 |
| cg23006519 | 7.13E−10 | 3.03E−04 | −0.249399298 | 0.249399298 | loss | 0.470718804 | 0.221319506 | LINC01101; LINC01101 | 37 | 2 |
| cg11613902 | 2.35E−09 | 9.98E−04 | −0.249316991 | 0.249316991 | loss | 0.681471674 | 0.432154682 | TLL2 | 37 | 10 |
| cg16511795 | 7.84E−10 | 3.33E−04 | −0.249312808 | 0.249312808 | loss | 0.640523308 | 0.3912105 | LRPAP1 | 37 | 4 |
| cg22816857 | 7.13E−10 | 3.03E−04 | −0.249305583 | 0.249305583 | loss | 0.933065583 | 0.68376 | ELAVL3; ELAVL3 | 37 | 19 |
| cg16009311 | 5.39E−09 | 2.29E−03 | −0.249282088 | 0.249282088 | loss | 0.674862694 | 0.425580606 | EXT1 | 37 | 8 |
| cg04779752 | 7.13E−10 | 3.03E−04 | −0.249281641 | 0.249281641 | loss | 0.9507667 | 0.701485059 | CLDN4 | 37 | 7 |
| cg16079541 | 7.13E−10 | 3.03E−04 | −0.249280623 | 0.249280623 | loss | 0.87522427 | 0.625943647 | | 37 | 6 |
| cg18019825 | 7.13E−10 | 3.03E−04 | −0.249253066 | 0.249253066 | loss | 0.957072877 | 0.707819812 | | 37 | 7 |
| cg25956097 | 7.13E−10 | 3.03E−04 | −0.249236386 | 0.249236386 | loss | 0.762786115 | 0.513549729 | ZNF32-AS3 | 37 | 10 |
| cg05134847 | 7.13E−10 | 3.03E−04 | −0.249232116 | 0.249232116 | loss | 0.404167351 | 0.154935235 | | 37 | 22 |
| cg19432362 | 6.30E−08 | 2.68E−02 | −0.249218672 | 0.249218672 | loss | 0.861208655 | 0.611989982 | SIAH3 | 37 | 13 |
| cg27355653 | 3.14E−08 | 1.33E−02 | −0.249201528 | 0.249201528 | loss | 0.638691481 | 0.389489953 | LINC00925 | 37 | 15 |
| cg12444736 | 1.01E−09 | 4.27E−04 | −0.24914009 | 0.24914009 | loss | 0.912339502 | 0.663199412 | | 37 | 13 |
| cg01016191 | 8.47E−10 | 3.60E−04 | −0.249103617 | 0.249103617 | loss | 0.420461653 | 0.171358036 | | 37 | 12 |
| cg25035485 | 7.77E−10 | 3.30E−04 | −0.249091149 | 0.249091149 | loss | 0.410459996 | 0.161368847 | APOB | 37 | 2 |
| cg15630458 | 7.13E−10 | 3.03E−04 | −0.249073273 | 0.249073273 | loss | 0.641702349 | 0.392629076 | | 37 | 8 |
| cg15986841 | 7.84E−10 | 3.33E−04 | −0.249069697 | 0.249069697 | loss | 0.437352348 | 0.188282651 | ECHDC2; ECHDC2; ECHDC2 | 37 | 1 |
| cg07861603 | 7.13E−10 | 3.03E−04 | −0.249066643 | 0.249066643 | loss | 0.414376168 | 0.165309525 | CCDC64B; LOC100128770 | 37 | 16 |
| cg09720117 | 5.84E−08 | 2.48E−02 | −0.249030689 | 0.249030689 | loss | 0.623791283 | 0.374760594 | KCNA5 | 37 | 12 |
| cg13460311 | 1.01E−09 | 4.27E−04 | −0.249021089 | 0.249021089 | loss | 0.694792289 | 0.4457712 | | 37 | 18 |
| cg08011245 | 1.68E−09 | 7.12E−04 | −0.248973877 | 0.248973877 | loss | 0.928444736 | 0.679470859 | | 37 | 19 |
| cg05815196 | 7.13E−10 | 3.03E−04 | −0.248964695 | 0.248964695 | loss | 0.398070255 | 0.149105559 | PLA2G3; PLA2G3 | 37 | 22 |
| cg05442477 | 8.58E−08 | 3.64E−02 | −0.248943925 | 0.248943925 | loss | 0.661287642 | 0.412343718 | OLIG1 | 37 | 21 |
| cg23553444 | 1.30E−09 | 5.52E−04 | −0.248939598 | 0.248939598 | loss | 0.841148304 | 0.592208706 | GLB1L2 | 37 | 11 |
| cg01055594 | 7.13E−10 | 3.03E−04 | −0.24890196 | 0.24890196 | loss | 0.750390166 | 0.501488206 | WDR35; WDR35 | 37 | 2 |
| cg02314896 | 7.13E−10 | 3.03E−04 | −0.248891932 | 0.248891932 | loss | 0.616407396 | 0.367515465 | SULT2B1 | 37 | 19 |
| cg10769767 | 8.47E−10 | 3.60E−04 | −0.248884471 | 0.248884471 | loss | 0.690457983 | 0.441573512 | PAX5; PAX5; PAX5; PAX5; PAX5; | 37 | 9 |
| | | | | | | | | PAX5; PAX5; PAX5; PAX5; | | |
| | | | | | | | | PAX5; PAX5; PAX5; PAX5 | | |
| cg27403609 | 1.54E−08 | 6.56E−03 | −0.24887671 | 0.24887671 | loss | 0.887499851 | 0.638623141 | | 37 | 2 |
| cg04279801 | 7.13E−10 | 3.03E−04 | −0.248876312 | 0.248876312 | loss | 0.615357836 | 0.366481524 | RNF112 | 37 | 17 |
| cg03460756 | 2.35E−09 | 9.98E−04 | −0.248811454 | 0.248811454 | loss | 0.792392783 | 0.543581329 | JTGB3 | 37 | 17 |
| cg03689146 | 1.19E−09 | 5.07E−04 | −0.248786491 | 0.248786491 | loss | 0.619305874 | 0.370519382 | | 37 | 7 |
| cg14036981 | 1.30E−09 | 5.52E−04 | −0.248779652 | 0.248779652 | loss | 0.667777864 | 0.418998212 | LOC338694 | 37 | 11 |
| cg00076774 | 7.13E−10 | 3.03E−04 | −0.248776547 | 0.248776547 | loss | 0.724026247 | 0.4752497 | | 37 | 2 |
| cg18821144 | 5.39E−09 | 2.29E−03 | −0.248764381 | 0.248764381 | loss | 0.969257645 | 0.720493265 | | 37 | 12 |
| cg10467371 | 1.03E−08 | 4.39E−03 | −0.248723532 | 0.248723532 | loss | 0.796576585 | 0.547853053 | | 37 | 6 |
| cg08994082 | 7.13E−10 | 3.03E−04 | −0.24871719 | 0.24871719 | loss | 0.914058113 | 0.665340924 | GNG7 | 37 | 19 |
| cg07777909 | 7.13E−10 | 3.03E−04 | −0.248698131 | 0.248698131 | loss | 0.89214796 | 0.643449829 | DMTN | 37 | 8 |
| cg21723861 | 1.42E−09 | 6.01E−04 | −0.248687389 | 0.248687389 | loss | 0.881010836 | 0.632323447 | | 37 | 17 |
| cg23268630 | 2.69E−08 | 1.14E−02 | −0.24868552 | 0.24868552 | loss | 0.739700202 | 0.491014682 | SPOCD1; SPOCD1; SPOCD1 | 37 | 1 |
| cg24506936 | 7.13E−10 | 3.03E−04 | −0.248667298 | 0.248667298 | loss | 0.654718598 | 0.4060513 | | 37 | 8 |
| cg19331988 | 1.12E−08 | 4.76E−03 | −0.24866556 | 0.24866556 | loss | 0.83403716 | 0.5853716 | | 37 | 2 |
| cg27239280 | 7.13E−10 | 3.03E−04 | −0.24858446 | 0.24858446 | loss | 0.448917566 | 0.200333106 | CTF1; CTF1 | 37 | 16 |
| cg20636351 | 1.99E−09 | 8.43E−04 | −0.248581138 | 0.248581138 | loss | 0.791875143 | 0.543294006 | | 37 | 8 |
| cg03440414 | 8.47E−10 | 3.60E−04 | −0.248543263 | 0.248543263 | loss | 0.79176624 | 0.543222976 | | 37 | 2 |
| cg14266237 | 2.30E−05 | 9.75E−03 | −0.248515831 | 0.248515831 | loss | 0.61334826 | 0.364832429 | KLF1 | 37 | 19 |
| cg11651209 | 7.13E−10 | 3.03E−04 | −0.248481538 | 0.248481538 | loss | 0.441663634 | 0.193182096 | | 37 | 12 |
| cg22277296 | 7.13E−10 | 3.03E−04 | −0.248456876 | 0.248456876 | loss | 0.688752906 | 0.440296029 | | 37 | 5 |
| cg09225287 | 1.99E−09 | 8.43E−04 | −0.248454892 | 0.248454892 | loss | 0.424699606 | 0.176244714 | SEMA3C | 37 | 7 |
| cg15142430 | 7.13E−10 | 3.03E−04 | −0.248449398 | 0.248449398 | loss | 0.589748398 | 0.341299 | PIAS2; PIAS2 | 37 | 18 |
| cg05520687 | 7.84E−10 | 3.33E−04 | −0.248446302 | 0.248446302 | loss | 0.756791473 | 0.508345171 | | 37 | 15 |
| cg06706876 | 7.13E−10 | 3.03E−04 | −0.248403819 | 0.248403819 | loss | 0.752186713 | 0.503882894 | PLLP | 37 | 16 |
| cg08415998 | 7.13E−10 | 3.03E−04 | −0.248325615 | 0.248325615 | loss | 0.556914762 | 0.308589147 | DRGX | 37 | 10 |
| cg18690729 | 9.23E−10 | 3.92E−04 | −0.248322555 | 0.248322555 | loss | 0.735919949 | 0.487597394 | | 37 | 10 |
| cg21697812 | 2.35E−09 | 9.98E−04 | −0.248304263 | 0.248304263 | loss | 0.548265204 | 0.299960941 | C11orf91 | 37 | 11 |
| cg03467555 | 9.23E−10 | 3.92E−04 | −0.248303936 | 0.248303936 | loss | 0.463275536 | 0.2149716 | | 37 | 16 |
| cg14989226 | 5.85E−09 | 2.49E−03 | −0.248303328 | 0.248303328 | loss | 0.403424291 | 0.155120962 | SRRM3 | 37 | 7 |
| cg18104354 | 2.56E−09 | 1.09E−03 | −0.248273549 | 0.248273549 | loss | 0.753859608 | 0.505586059 | | 37 | 10 |
| cg06862049 | 1.18E−08 | 5.01E−03 | −0.248240565 | 0.248240565 | loss | 0.659093094 | 0.410852529 | LMTK3 | 37 | 19 |
| cg27340001 | 7.13E−10 | 3.03E−04 | −0.248228549 | 0.248228549 | loss | 0.833220643 | 0.584992094 | TP53BP1; TP53BP1; TP53BP1; | 37 | 15 |
| | | | | | | | | RNU6-28P | | |
| cg03027739 | 7.13E−10 | 3.03E−04 | −0.248228243 | 0.248228243 | loss | 0.653731513 | 0.405503271 | | 37 | 7 |
| cg18534077 | 7.13E−10 | 3.03E−04 | −0.248187932 | 0.248187932 | loss | 0.788315738 | 0.540127806 | AS3MT; C10orf32-ASMT | 37 | 10 |
| cg17307791 | 1.42E−09 | 6.01E−04 | −0.248185097 | 0.248185097 | loss | 0.693170321 | 0.444985224 | | 37 | 4 |
| cg11312495 | 1.01E−09 | 4.27E−04 | −0.248173969 | 0.248173969 | loss | 0.846159634 | 0.597985665 | | 37 | 5 |
| cg22356117 | 9.23E−10 | 3.92E−04 | −0.248161218 | 0.248161218 | loss | 0.824581353 | 0.576420135 | LAMP3 | 37 | 3 |
| cg25418852 | 5.80E−09 | 2.46E−03 | −0.248156211 | 0.248156211 | loss | 0.847735292 | 0.599579081 | SCGB1B2P | 37 | 19 |
| cg14302936 | 2.56E−09 | 1.09E−03 | −0.248099535 | 0.248099535 | loss | 0.637311206 | 0.389211671 | GLIPR2 | 37 | 9 |
| cg17516330 | 7.13E−10 | 3.03E−04 | −0.248089622 | 0.248089622 | loss | 0.730504181 | 0.482414559 | STRA6; STRA6; STRA6; STRA6; | 37 | 15 |
| | | | | | | | | STRA6; STRA6; STRA6; STRA6 | | |
| cg24117274 | 7.13E−10 | 3.03E−04 | −0.248085121 | 0.248085121 | loss | 0.278803238 | 0.030718116 | RAP1GAP; RAP1GAP; RAP1GAP | 37 | 1 |
| cg10969241 | 3.02E−09 | 1.28E−03 | −0.248068593 | 0.248068593 | loss | 0.718299428 | 0.470230835 | | 37 | 10 |
| cg16810343 | 3.88E−09 | 1.65E−03 | −0.248064928 | 0.248064928 | loss | 0.44335944 | 0.195294512 | SRRM3 | 37 | 7 |
| cg03846408 | 7.13E−10 | 3.03E−04 | −0.248060825 | 0.248060825 | loss | 0.530763819 | 0.282702994 | IFITM10; MOB2 | 37 | 11 |
| cg01654820 | 7.13E−10 | 3.03E−04 | −0.248055304 | 0.248055304 | loss | 0.730180204 | 0.4821249 | EMP2 | 37 | 16 |
| cg07209550 | 7.77E−10 | 3.30E−04 | −0.248033954 | 0.248033954 | loss | 0.761694813 | 0.513660859 | CMYA5 | 37 | 5 |
| cg09982773 | 7.13E−10 | 3.03E−04 | −0.247985559 | 0.247985559 | loss | 0.893470836 | 0.645485276 | KCP; KCP; KCP; KCP | 37 | 7 |
| cg18498241 | 7.84E−10 | 3.33E−04 | −0.247980891 | 0.247980891 | loss | 0.343684206 | 0.095703315 | | 37 | 11 |
| cg19824710 | 2.16E−09 | 9.17E−04 | −0.247916907 | 0.247916907 | loss | 0.763338819 | 0.515421912 | PTPRF; PTPRF | 37 | 1 |
| cg15151749 | 7.13E−10 | 3.03E−04 | −0.247913776 | 0.247913776 | loss | 0.796807723 | 0.548893947 | USH1C; USH1C | 37 | 11 |
| cg07421596 | 7.13E−10 | 3.03E−04 | −0.247903928 | 0.247903928 | loss | 0.555267798 | 0.307363871 | CFB | 37 | 6 |
| cg23886216 | 7.13E−10 | 3.03E−04 | −0.247738756 | 0.247738756 | loss | 0.605045715 | 0.357306959 | | 37 | 14 |
| cg04983977 | 7.13E−10 | 3.03E−04 | −0.247724318 | 0.247724318 | loss | 0.731808583 | 0.484084265 | GPR25 | 37 | 1 |
| cg08750432 | 8.47E−10 | 3.60E−04 | −0.247707695 | 0.247707695 | loss | 0.597048672 | 0.349340976 | | 37 | 8 |
| cg02931642 | 2.35E−09 | 9.98E−04 | −0.247684947 | 0.247684947 | loss | 0.660104447 | 0.4124195 | | 37 | 3 |
| cg01135546 | 7.13E−10 | 3.03E−04 | −0.247627359 | 0.247627359 | loss | 0.301240355 | 0.053612995 | | 37 | 17 |
| cg12159314 | 7.13E−10 | 3.03E−04 | −0.247617532 | 0.247617532 | loss | 0.689806091 | 0.442188559 | SLC24A4 | 37 | 14 |
| cg19247654 | 5.01E−08 | 2.13E−02 | −0.247609083 | 0.247609083 | loss | 0.857201736 | 0.609592653 | | 37 | 18 |
| cg04798919 | 9.23E−10 | 3.92E−04 | −0.247608726 | 0.247608726 | loss | 0.825489779 | 0.577881053 | ZNF575 | 37 | 19 |
| cg07972116 | 7.77E−10 | 3.30E−04 | −0.247580352 | 0.247580352 | loss | 0.67119737 | 0.423617018 | | 37 | 1 |
| cg21531689 | 7.13E−10 | 3.03E−04 | −0.247565121 | 0.247565121 | loss | 0.737050562 | 0.489485441 | DNPEP | 37 | 2 |
| cg05090695 | 7.13E−10 | 3.03E−04 | −0.247552669 | 0.247552669 | loss | 0.402778974 | 0.155226305 | CDKN1C; CDKN1C; CDKN1C | 37 | 11 |
| cg10245967 | 7.13E−10 | 3.03E−04 | −0.247527472 | 0.247527472 | loss | 0.827040525 | 0.579513053 | | 37 | 1 |
| cg19241831 | 8.47E−10 | 3.60E−04 | −0.247494288 | 0.247494288 | loss | 0.843364511 | 0.595870224 | OTOF | 37 | 2 |
| cg17938867 | 7.13E−10 | 3.03E−04 | −0.247477861 | 0.247477861 | loss | 0.889433143 | 0.641955282 | GPSM2 | 37 | 1 |
| cg06023661 | 1.01E−09 | 4.27E−04 | −0.247476657 | 0.247476657 | loss | 0.431773568 | 0.184296911 | NOTCH4 | 37 | 6 |
| cg06035600 | 1.03E−08 | 4.39E−03 | −0.247443831 | 0.247443831 | loss | 0.546441964 | 0.298998133 | MAP3K6 | 37 | 1 |
| cg19443750 | 3.40E−08 | 1.44E−02 | −0.247419656 | 0.247419656 | loss | 0.803874726 | 0.556455071 | | 37 | 13 |
| cg13019568 | 2.25E−09 | 9.55E−04 | −0.247407117 | 0.247407117 | loss | 0.352529974 | 0.105122856 | | 37 | 14 |
| cg04709703 | 7.13E−10 | 3.03E−04 | −0.247386077 | 0.247386077 | loss | 0.824476425 | 0.577090347 | DAGLA | 37 | 11 |
| cg17019053 | 7.13E−10 | 3.03E−04 | −0.247344584 | 0.247344584 | loss | 0.306066609 | 0.058722026 | M1AP; M1AP; M1AP; M1AP | 37 | 2 |
| cg13293535 | 2.56E−09 | 1.09E−03 | −0.247337352 | 0.247337352 | loss | 0.587415934 | 0.340078582 | GATA4 | 37 | 8 |
| cg14339848 | 7.13E−10 | 3.03E−04 | −0.247313015 | 0.247313015 | loss | 0.912799468 | 0.665486453 | KLF15 | 37 | 3 |
| cg21904329 | 4.57E−09 | 1.94E−03 | −0.247303874 | 0.247303874 | loss | 0.749527721 | 0.502223847 | | 37 | 15 |
| cg02240291 | 7.13E−10 | 3.03E−04 | −0.247303326 | 0.247303326 | loss | 0.893399232 | 0.646095906 | SMARCD3; SMARCD3; SMARCD3 | 37 | 7 |
| cg10056813 | 7.77E−10 | 3.30E−04 | −0.247286101 | 0.247286101 | loss | 0.788679372 | 0.541393271 | TTLL10 | 37 | 1 |
| cg14492992 | 3.88E−09 | 1.65E−03 | −0.247246248 | 0.247246248 | loss | 0.707790836 | 0.460544588 | | 37 | 15 |
| cg16432009 | 1.73E−09 | 7.34E−04 | −0.247221361 | 0.247221361 | loss | 0.950010292 | 0.702788931 | CDC42EP1 | 37 | 22 |
| cg10997906 | 7.13E−10 | 3.03E−04 | −0.247212717 | 0.247212717 | loss | 0.648073806 | 0.400861088 | ADAMTS2; ADAMTS2 | 37 | 5 |
| cg02687418 | 4.97E−09 | 2.11E−03 | −0.2472003 | 0.2472003 | loss | 0.349610819 | 0.102410519 | | 37 | 11 |
| cg21023520 | 2.35E−09 | 9.98E−04 | −0.247193552 | 0.247193552 | loss | 0.898064811 | 0.650871259 | MPPED1 | 37 | 22 |
| cg04021544 | 2.12E−08 | 9.01E−03 | −0.247180602 | 0.247180602 | loss | 0.641160549 | 0.393979947 | | 37 | 7 |
| cg07138994 | 1.30E−09 | 5.52E−04 | −0.247158535 | 0.247158535 | loss | 0.686873153 | 0.439714618 | | 37 | 11 |
| cg09565002 | 2.30E−08 | 9.75E−03 | −0.247151635 | 0.247151635 | loss | 0.802263 | 0.555111365 | FGFBP1 | 37 | 4 |
| cg20903926 | 7.13E−10 | 3.03E−04 | −0.24713472 | 0.24713472 | loss | 0.899707185 | 0.652572465 | C1orf177; C1orf177 | 37 | 1 |
| cg18699242 | 1.89E−09 | 8.02E−04 | −0.247107001 | 0.247107001 | loss | 0.470029651 | 0.22292265 | USP18 | 37 | 22 |
| cg16703135 | 7.77E−10 | 3.30E−04 | −0.247103337 | 0.247103337 | loss | 0.51075346 | 0.263650124 | | 37 | 7 |
| cg10765171 | 7.77E−10 | 3.30E−04 | −0.247100332 | 0.247100332 | loss | 0.804494474 | 0.557394141 | | 37 | 13 |
| cg23366166 | 7.13E−10 | 3.03E−04 | −0.247084339 | 0.247084339 | loss | 0.552001351 | 0.304917012 | FEZ1; FEZ1 | 37 | 11 |
| cg12944343 | 1.42E−09 | 6.01E−04 | −0.247061732 | 0.247061732 | loss | 0.540944243 | 0.293882512 | | 37 | 14 |
| cg14544666 | 7.13E−10 | 3.03E−04 | −0.247039292 | 0.247039292 | loss | 0.298523351 | 0.051484059 | RASGEF1C | 37 | 5 |
| cg00262344 | 1.01E−09 | 4.27E−04 | −0.247014485 | 0.247014485 | loss | 0.705420485 | 0.458406 | PSMA8; PSMA8; PSMA8 | 37 | 18 |
| cg13476313 | 9.23E−10 | 3.92E−04 | −0.245993971 | 0.246993971 | loss | 0.813283794 | 0.566289824 | NR5A1 | 37 | 9 |
| cg00116315 | 8.80E−09 | 3.74E−03 | −0.2469841 | 0.2469841 | loss | 0.525374311 | 0.278390212 | TEAD3; TULP1 | 37 | 6 |
| cg19479373 | 8.11E−09 | 3.44E−03 | −0.246976338 | 0.246976338 | loss | 0.708023774 | 0.461047435 | CRTAC1; CRTAC1 | 37 | 10 |
| cg15523463 | 9.54E−09 | 4.05E−03 | −0.246952757 | 0.246952757 | loss | 0.793863192 | 0.546910435 | | 37 | 5 |
| cg25736663 | 1.42E−09 | 6.01E−04 | −0.246942918 | 0.246942918 | loss | 0.803323294 | 0.556380376 | SLC35F3 | 37 | 1 |
| cg07928883 | 8.47E−10 | 3.60E−04 | −0.246927557 | 0.246927557 | loss | 0.820862404 | 0.573934847 | | 37 | 7 |
| cg06472166 | 5.39E−09 | 2.29E−03 | −0.246877125 | 0.246877125 | loss | 0.36772047 | 0.120843345 | | 37 | 7 |
| cg24123106 | 1.99E−09 | 8.43E−04 | −0.246857463 | 0.246857463 | loss | 0.837974034 | 0.591116571 | FABP7 | 37 | 6 |
| cg00904823 | 2.48E−08 | 1.05E−02 | −0.246848524 | 0.246848524 | loss | 0.802014789 | 0.555166265 | RBM20 | 37 | 10 |
| cg05129050 | 8.11E−09 | 3.44E−03 | −0.246844923 | 0.246844923 | loss | 0.540958123 | 0.2941132 | CAMTA1 | 37 | 1 |
| cg23739457 | 1.19E−09 | 5.07E−04 | −0.246836936 | 0.246836936 | loss | 0.587201394 | 0.340364459 | DSCAML1 | 37 | 11 |
| cg05670995 | 7.13E−10 | 3.03E−04 | −0.246812993 | 0.246812993 | loss | 0.688170387 | 0.441357394 | MMP21 | 37 | 10 |
| cg16273979 | 9.22E−08 | 3.91E−02 | −0.246798629 | 0.246798629 | loss | 0.745215023 | 0.498416394 | | 37 | 4 |
| cg05113697 | 7.13E−10 | 3.03E−04 | −0.246795352 | 0.246795352 | loss | 0.647854664 | 0.401059312 | | 37 | 16 |
| cg23765086 | 7.77E−10 | 3.30E−04 | −0.246759377 | 0.246759377 | loss | 0.629470983 | 0.382711606 | CAMK2B; CAMK2B; CAMK2B; | 37 | 7 |
| | | | | | | | | CAMK2B; CAMK2B; CAMK2B; | | |
| | | | | | | | | CAMK2B; CAMK2B | | |
| cg02082083 | 1.68E−09 | 7.12E−04 | −0.246718515 | 0.246718515 | loss | 0.796910774 | 0.550192259 | PLA2G5 | 37 | 1 |
| cg21629821 | 6.35E−09 | 2.70E−03 | 0.246702624 | 0.246702624 | gain | 0.386674664 | 0.633377288 | DKFZp434J0226 | 37 | 19 |
| cg14847483 | 7.13E−10 | 3.03E−04 | −0.246696411 | 0.246696411 | loss | 0.379368136 | 0.132671725 | EMC4; EMC4 | 37 | 15 |
| cg20077028 | 2.48E−08 | 1.05E−02 | −0.246645019 | 0.246645019 | loss | 0.589143513 | 0.342498494 | | 37 | 12 |
| cg13778078 | 1.42E−09 | 6.01E−04 | −0.246641062 | 0.246641062 | loss | 0.887226462 | 0.6405854 | CPLX1 | 37 | 4 |
| cg07525299 | 5.85E−09 | 2.49E−03 | −0.246634866 | 0.246634866 | loss | 0.759854666 | 0.5132198 | ASCL2 | 37 | 11 |
| cg05306173 | 7.77E−10 | 3.30E−04 | −0.246628331 | 0.246628331 | loss | 0.664904272 | 0.418275941 | KIAA1598 | 37 | 10 |
| cg21428990 | 4.26E−08 | 1.81E−02 | 0.24662128 | 0.24662128 | gain | 0.078136996 | 0.324758276 | | 37 | 7 |
| cg03421300 | 7.13E−10 | 3.03E−04 | −0.246619433 | 0.246519433 | loss | 0.900054757 | 0.653435324 | SFN | 37 | 1 |
| cg22038738 | 3.67E−08 | 1.56E−02 | −0.246613981 | 0.246613981 | loss | 0.800525392 | 0.553911412 | PLAT; PLAT | 37 | 8 |
| cg23621967 | 7.13E−10 | 3.03E−04 | −0.246602319 | 0.246602319 | loss | 0.336846657 | 0.090244338 | | 37 | 1 |
| cg02238549 | 7.48E−09 | 3.18E−03 | −0.246592911 | 0.246592911 | loss | 0.434301628 | 0.187708718 | | 37 | 10 |
| cg00023919 | 7.13E−10 | 3.03E−04 | −0.246591153 | 0.246591153 | loss | 0.424513853 | 0.1779227 | RHOD | 37 | 11 |
| cg17149262 | 1.16E−07 | 4.91E−02 | −0.246578962 | 0.246578962 | loss | 0.576641162 | 0.3300622 | | 37 | 8 |
| cg07856975 | 1.96E−08 | 8.33E−03 | −0.246536121 | 0.246536121 | loss | 0.633494168 | 0.386958047 | ETV7; ETV7; ETV7; ETV7; ETV7; | 37 | 6 |
| | | | | | | | | ETV7; ETV7; ETV7 | | |
| cg18730926 | 7.77E−10 | 3.30E−04 | −0.246528889 | 0.246528889 | loss | 0.580197489 | 0.3336686 | PCDP1; PCDP1 | 37 | 2 |
| cg04258219 | 7.13E−10 | 3.03E−04 | −0.246507712 | 0.246507712 | loss | 0.320709613 | 0.074201901 | | 37 | 7 |
| cg21079003 | 1.32E−08 | 5.59E−03 | −0.246497438 | 0.246497438 | loss | 0.4761719 | 0.229674462 | RGMA; RGMA; RGMA; RGMA; | 37 | 15 |
| | | | | | | | | RGMA; RGMA | | |
| cg06071746 | 1.10E−09 | 4.65E−04 | −0.246452143 | 0.246452143 | loss | 0.522188766 | 0.275736624 | GDPD1; GDPD1; GDPD1 | 37 | 17 |
| cg27598583 | 7.77E−10 | 3.30E−04 | −0.246398116 | 0.246398116 | loss | 0.954402745 | 0.708004629 | | 37 | 16 |
| cg13826247 | 7.13E−10 | 3.03E−04 | −0.246388917 | 0.246388917 | loss | 0.414375317 | 0.1679864 | HOXC6; HOXC4; HOXC6; HOXC5 | 37 | 12 |
| cg09584358 | 9.23E−10 | 3.92E−04 | −0.246370315 | 0.246370315 | loss | 0.743209026 | 0.496838712 | | 37 | 17 |
| cg03721641 | 7.13E−10 | 3.03E−04 | −0.246347808 | 0.246347808 | loss | 0.808266338 | 0.561918529 | IL17REL; IL17REL | 37 | 22 |
| cg03874542 | 1.81E−08 | 7.69E−03 | −0.246329638 | 0.246329638 | loss | 0.864688426 | 0.618358788 | | 37 | 10 |
| cg01321174 | 7.77E−10 | 3.30E−04 | −0.246279794 | 0.246279794 | loss | 0.728118758 | 0.481838965 | | 37 | 4 |
| cg24231558 | 2.69E−08 | 1.14E−02 | −0.246266706 | 0.246266706 | loss | 0.765509989 | 0.519243282 | | 37 | 7 |
| cg14023999 | 1.42E−09 | 6.01E−04 | −0.246265347 | 0.246265347 | loss | 0.650485458 | 0.404220112 | | 37 | 15 |
| cg05766510 | 3.97E−08 | 1.69E−02 | −0.246239377 | 0.246239377 | loss | 0.804605947 | 0.558366571 | PTPRN2; PTPRN2; PTPRN2 | 37 | 7 |
| cg24553170 | 1.10E−09 | 4.65E−04 | −0.246215776 | 0.246215776 | loss | 0.651734617 | 0.405518841 | BEND5; AGBL4 | 37 | 1 |
| cg05336956 | 7.13E−10 | 3.03E−04 | −0.246212635 | 0.246212635 | loss | 0.405039258 | 0.158826624 | | 37 | 1 |
| cg26839356 | 7.13E−10 | 3.03E−04 | −0.246207358 | 0.246207358 | loss | 0.43194037 | 0.185733012 | | 37 | 15 |
| cg27532318 | 7.77E−10 | 3.30E−04 | −0.246101335 | 0.246101335 | loss | 0.376104047 | 0.130002712 | | 37 | 7 |
| cg11972810 | 7.84E−10 | 3.33E−04 | −0.246077857 | 0.246077857 | loss | 0.520011615 | 0.273933759 | C15orf27 | 37 | 15 |
| Cg01099024 | 3.14E−08 | 1.33E−02 | −0.246070334 | 0.246070334 | loss | 0.539177875 | 0.293107541 | SMOC1; SMOC1 | 37 | 14 |
| cg02758247 | 2.56E−09 | 1.09E−03 | −0.246053036 | 0.246053036 | loss | 0.713594077 | 0.467541041 | CDH20 | 37 | 18 |
| cg14413262 | 9.23E−10 | 3.92E−04 | −0.246000184 | 0.246000184 | loss | 0.878877019 | 0.632876835 | SLC45A1 | 37 | 1 |
| cg03268942 | 1.83E−09 | 7.75E−04 | −0.245994131 | 0.245994131 | loss | 0.73649886 | 0.490504729 | | 37 | 2 |
| cg27336261 | 7.77E−10 | 3.30E−04 | −0.245986262 | 0.245986262 | loss | 0.835954109 | 0.589967847 | | 37 | 10 |
| cg07201996 | 5.41E−08 | 2.30E−02 | −0.245974284 | 0.245974284 | loss | 0.750093249 | 0.504118965 | CDH4; CDH4; CDH4 | 37 | 20 |
| cg26411822 | 7.13E−10 | 3.03E−04 | −0.245958064 | 0.245958064 | loss | 0.346900579 | 0.100942515 | STX12 | 37 | 1 |
| cg24125828 | 1.10E−09 | 4.65E−04 | −0.245939151 | 0.245939151 | loss | 0.478947551 | 0.2330084 | PRRT1 | 37 | 6 |
| cg11858450 | 3.57E−09 | 1.52E−03 | −0.245905058 | 0.245905058 | loss | 0.758724975 | 0.512819918 | CCDC105 | 37 | 19 |
| cg15676542 | 1.83E−09 | 7.75E−04 | −0.245873538 | 0.245873538 | loss | 0.623638738 | 0.3777652 | BTN2A3P | 37 | 6 |
| cg22830895 | 8.47E−10 | 3.60E−04 | −0.245872155 | 0.245872155 | loss | 0.454962985 | 0.209090829 | CRYGN | 37 | 7 |
| cg04359306 | 7.13E−10 | 3.03E−04 | −0.245824088 | 0.245824088 | loss | 0.604431153 | 0.358607065 | | 37 | 16 |
| cg02939356 | 8.47E−10 | 3.60E−04 | −0.245823879 | 0.245823879 | loss | 0.926107491 | 0.680283612 | PRSS42; PRSS42 | 37 | 3 |
| cg13679303 | 2.82E−08 | 1.20E−02 | −0.245814223 | 0.245814223 | loss | 0.360155511 | 0.114341288 | | 37 | 9 |
| cg22162848 | 7.13E−10 | 3.03E−04 | −0.245807481 | 0.245807481 | loss | 0.602053634 | 0.356246153 | ITGB4; ITGB4 | 37 | 17 |
| cg22079043 | 7.13E−10 | 3.03E−04 | −0.245787839 | 0.245787839 | loss | 0.525686768 | 0.279898929 | CTSD; MOB2; MOB2 | 37 | 11 |
| cg25705148 | 1.12E−08 | 4.76E−03 | −0.245771997 | 0.245771997 | loss | 0.518671413 | 0.272899416 | | 37 | 2 |
| cg15154047 | 7.13E−10 | 3.03E−04 | −0.245755998 | 0.245755998 | loss | 0.574258992 | 0.328502994 | | 37 | 8 |
| cg23013473 | 8.47E−10 | 3.60E−04 | −0.245703744 | 0.245703744 | loss | 0.856061591 | 0.610357847 | | 37 | 17 |
| cg23345697 | 1.99E−09 | 8.43E−04 | −0.245676961 | 0.245676961 | loss | 0.743871655 | 0.498194694 | | 37 | 7 |
| cg17032564 | 1.10E−09 | 4.65E−04 | −0.245608653 | 0.245608653 | loss | 0.536709289 | 0.291100635 | | 37 | 5 |
| cg20113012 | 7.13E−10 | 3.03E−04 | −0.245587104 | 0.245587104 | loss | 0.361391196 | 0.115804092 | CDC42EP4 | 37 | 17 |
| cg17841545 | 7.13E−10 | 3.03E−04 | −0.245572891 | 0.245572891 | loss | 0.852894474 | 0.607321582 | NRAP; NRAP; NRAP | 37 | 10 |
| cg06917617 | 7.13E−10 | 3.03E−04 | −0.245571417 | 0.245571417 | loss | 0.382858058 | 0.137286641 | CR1L | 37 | 1 |
| cg00034755 | 2.16E−09 | 9.17E−04 | −0.245551179 | 0.245551179 | loss | 0.626238932 | 0.380687753 | | 37 | 22 |
| cg22755142 | 1.54E−09 | 6.54E−04 | −0.245531424 | 0.245531424 | loss | 0.818799547 | 0.573268124 | | 37 | 2 |
| cg25453625 | 1.42E−09 | 6.01E−04 | −0.245517979 | 0.245517979 | loss | 0.702219696 | 0.456701718 | | 37 | 7 |
| cg06962684 | 1.10E−09 | 4.65E−04 | −0.24550298 | 0.24550298 | loss | 0.763927198 | 0.518424218 | CELF4; CELF4; CELF4; CELF4 | 37 | 18 |
| cg06552810 | 6.89E−09 | 2.93E−03 | −0.245496467 | 0.245496467 | loss | 0.654536979 | 0.409040512 | | 37 | 11 |
| cg07041748 | 7.13E−10 | 3.03E−04 | −0.245495969 | 0.245495969 | loss | 0.719516145 | 0.474020176 | DOC2A; DOC2A; DOC2A; DOC2A; | 37 | 16 |
| | | | | | | | | DOC2A; DOC2A; DOC2A | | |
| cg05725404 | 9.54E−09 | 4.05E−03 | −0.245490224 | 0.245490224 | loss | 0.44177143 | 0.196281206 | NDRG4; NDRG4; NDRG4; NDRG4; | 37 | 16 |
| | | | | | | | | NDRG4; NDRG4; NDRG4; | | |
| | | | | | | | | NDRG4 | | |
| cg06901759 | 6.35E−09 | 2.70E−03 | −0.245479789 | 0.245479789 | loss | 0.592776883 | 0.347297094 | | 37 | 20 |
| cg22603450 | 1.83E−09 | 7.75E−04 | −0.245450655 | 0.245450655 | loss | 0.536022685 | 0.290572029 | | 37 | 19 |
| cg14059257 | 7.13E−10 | 3.03E−04 | −0.245447529 | 0.245447529 | loss | 0.633129911 | 0.387682382 | KRT7 | 37 | 12 |
| cg14013080 | 7.13E−10 | 3.03E−04 | −0.245438001 | 0.245438001 | loss | 0.505812319 | 0.260374318 | | 37 | 14 |
| cg03077077 | 7.84E−10 | 3.33E−04 | −0.245426605 | 0.245426606 | loss | 0.6731587 | 0.427732094 | TMEM155; PP12613 | 37 | 4 |
| cg14395744 | 1.42E−09 | 6.01E−04 | −0.245423589 | 0.245423589 | loss | 0.755425825 | 0.510002235 | | 37 | 18 |
| cg03895348 | 1.00E−08 | 4.25E−03 | −0.245420023 | 0.245420023 | loss | 0.739349517 | 0.493929494 | CD207 | 37 | 2 |
| cg06682185 | 1.01E−09 | 4.27E−04 | −0.24541745 | 0.24541745 | loss | 0.830267332 | 0.584849882 | SERPINB5 | 37 | 18 |
| cg03490759 | 6.32E−09 | 2.68E−03 | −0.245414105 | 0.245414105 | loss | 0.590752511 | 0.345338406 | HHIPL1 | 37 | 14 |
| cg12669395 | 7.77E−10 | 3.30E−04 | −0.245398113 | 0.245398113 | loss | 0.73184846 | 0.486450347 | DDR1; DDR1; DDR1; DDR1; | 37 | 6 |
| | | | | | | | | DDR1; DDR1 | | |
| cg25314315 | 1.01E−09 | 4.27E−04 | −0.245397848 | 0.245397848 | loss | 0.793659319 | 0.548261471 | RHBDL2; RHBDL2 | 37 | 1 |
| cg13825930 | 1.54E−09 | 6.54E−04 | −0.245379443 | 0.245379443 | loss | 0.341437185 | 0.096057742 | | 37 | X |
| cg10107890 | 3.67E−08 | 1.56E−02 | −0.245369112 | 0.245369112 | loss | 0.701066389 | 0.455697276 | | 37 | 2 |
| cg11975790 | 6.89E−09 | 2.93E−03 | −0.245363335 | 0.245363335 | loss | 0.615540664 | 0.370177329 | DDR1; DDR1; DDR1 | 37 | 6 |
| cg10533103 | 4.57E−09 | 1.94E−03 | −0.245351682 | 0.245351682 | loss | 0.777177917 | 0.531826235 | | 37 | 1 |
| cg15638969 | 1.99E−09 | 8.43E−04 | −0.245307304 | 0.245307304 | loss | 0.827626257 | 0.582318953 | | 37 | 10 |
| cg01765164 | 2.35E−09 | 9.98E−04 | −0.245238827 | 0.245238827 | loss | 0.453614157 | 0.208375329 | | 37 | 16 |
| cg14308254 | 7.77E−10 | 3.30E−04 | −0.245230801 | 0.245230801 | loss | 0.920994036 | 0.675763235 | FAM83A; FAM83A | 37 | 8 |
| cg10096100 | 7.48E−09 | 3.18E−03 | −0.245193639 | 0.245193639 | loss | 0.674795151 | 0.429601512 | CNKSR1; CNKSR1 | 37 | 1 |
| cg08275704 | 7.13E−10 | 3.03E−04 | −0.24512145 | 0.24512145 | loss | 0.691022885 | 0.445901435 | | 37 | 19 |
| cg09782034 | 8.55E−08 | 3.63E−02 | −0.245103626 | 0.245103626 | loss | 0.765802585 | 0.520698959 | RORA | 37 | 15 |
| cg27305917 | 7.13E−10 | 3.03E−04 | −0.245079089 | 0.245079089 | loss | 0.449759436 | 0.204680347 | | 37 | 21 |
| cg19084479 | 9.23E−10 | 3.92E−04 | −0.245077172 | 0.245077172 | loss | 0.456218608 | 0.211141435 | POLD2; POLD2; POLD2 | 37 | 7 |
| cg08149865 | 7.84E−10 | 3.33E−04 | −0.245069302 | 0.245069302 | loss | 0.740544619 | 0.495475318 | DMTN; DMTN; DMTN; DMTN | 37 | 8 |
| cg11344005 | 1.19E−09 | 5.07E−04 | −0.245032695 | 0.245032695 | loss | 0.549960177 | 0.304927482 | KLHDC7B | 37 | 22 |
| cg17055821 | 7.13E−10 | 3.03E−04 | −0.245029811 | 0.245029811 | loss | 0.328061826 | 0.083032016 | C17orf75 | 37 | 17 |
| cg02736908 | 7.13E−10 | 3.03E−04 | −0.245001865 | 0.245001865 | loss | 0.684072483 | 0.439070618 | | 37 | 17 |
| cg25136988 | 1.19E−09 | 5.07E−04 | −0.244970747 | 0.244970747 | loss | 0.759057653 | 0.514086906 | LY6G5C | 37 | 6 |
| cg09404429 | 7.13E−10 | 3.03E−04 | −0.244954313 | 0.244954313 | loss | 0.512489925 | 0.267535612 | | 37 | 17 |
| cg07842521 | 7.13E−10 | 3.03E−04 | −0.244911842 | 0.244911842 | loss | 0.72875843 | 0.483846588 | | 37 | 16 |
| cg12847793 | 7.13E−10 | 3.03E−04 | −0.244881594 | 0.244881594 | loss | 0.673570642 | 0.428689047 | DDR1; DDR1; DDR1 | 37 | 6 |
| cg09649266 | 7.13E−10 | 3.03E−04 | −0.244873868 | 0.244873868 | loss | 0.619295262 | 0.374421394 | | 37 | 2 |
| cg23579185 | 2.86E−09 | 1.22E−03 | −0.244803246 | 0.244803246 | loss | 0.804419535 | 0.559616288 | | 37 | 18 |
| cg24870966 | 1.10E−09 | 4.65E−04 | −0.244801327 | 0.244801327 | loss | 0.714521321 | 0.469719994 | LINC00482; LINC00482 | 37 | 17 |
| cg02718966 | 7.13E−10 | 3.03E−04 | −0.24478524 | 0.24478524 | loss | 0.341177989 | 0.096392749 | HIGD1A; HIGD1A; HIGD1A | 37 | 3 |
| cg15883853 | 1.67E−08 | 7.10E−03 | −0.244775745 | 0.244775745 | loss | 0.860312598 | 0.615536853 | | 37 | 12 |
| cg12645284 | 7.77E−10 | 3.30E−04 | −0.244713259 | 0.244713259 | loss | 0.856970794 | 0.612257535 | ADAMTS7 | 37 | 15 |
| cg21514735 | 7.48E−09 | 3.18E−03 | −0.244658071 | 0.244658071 | loss | 0.500269577 | 0.255611506 | LOC100505532 | 37 | 8 |
| cg09622269 | 7.13E−10 | 3.03E−04 | −0.244655289 | 0.244655289 | loss | 0.536600442 | 0.291945153 | DUPD1 | 37 | 10 |
| cg02389590 | 7.13E−10 | 3.03E−04 | −0.244631096 | 0.244631096 | loss | 0.534032113 | 0.289401018 | SYBU; SYBU; SYBU; SYBU; SYBU; | 37 | 8 |
| | | | | | | | | SYBU; SYBU; SYBU; SYBU; | | |
| | | | | | | | | SYBU; SYBU; SYBU; SYBU; | | |
| | | | | | | | | SYBU | | |
| cg250S0332 | 9.45E−10 | 4.01E−04 | −0.244618929 | 0.244618929 | loss | 0.636581124 | 0.391962194 | | 37 | 11 |
| cg07943780 | 7.13E−10 | 3.03E−04 | −0.244606755 | 0.244606755 | loss | 0.351092779 | 0.106486024 | | 37 | 8 |
| cg17024542 | 6.35E−09 | 2.70E−03 | −0.244568999 | 0.244568999 | loss | 0.781739781 | 0.537170782 | CHST2 | 37 | 3 |
| cg12850379 | 8.47E−10 | 3.60E−04 | −0.244560893 | 0.244560893 | loss | 0.564896023 | 0.320335129 | TCEA1; TCEA1 | 37 | 8 |
| cg13844049 | 7.13E−10 | 3.03E−04 | −0.244553502 | 0.244553502 | loss | 0.356378815 | 0.111825313 | | 37 | 1 |
| cg23904955 | 3.28E−09 | 1.39E−03 | −0.244552158 | 0.244552158 | loss | 0.657535075 | 0.412982918 | | 37 | 10 |
| cg20372230 | 1.10E−09 | 4.65E−04 | −0.244540528 | 0.244540528 | loss | 0.380551908 | 0.13601138 | | 37 | 7 |
| cg13879655 | 7.77E−10 | 3.30E−04 | −0.244537101 | 0.244537101 | loss | 0.833308225 | 0.588771124 | | 37 | 8 |
| cg12887581 | 4.64E−08 | 1.97E−02 | −0.244529852 | 0.244529852 | loss | 0.782998911 | 0.538469059 | | 37 | 10 |
| cg14071650 | 7.13E−10 | 3.03E−04 | −0.244518186 | 0.244518186 | loss | 0.450795108 | 0.206276921 | LMX1B; LMX1B; LMX1B | 37 | 9 |
| cg27503950 | 8.11E−09 | 3.44E−03 | −0.244507358 | 0.244507358 | loss | 0.665806317 | 0.421298959 | | 37 | 6 |
| cg05225012 | 7.84E−10 | 3.33E−04 | −0.244466338 | 0.244466338 | loss | 0.948488656 | 0.704022318 | | 37 | 2 |
| cg06264679 | 7.13E−10 | 3.03E−04 | −0.244432465 | 0.244432465 | loss | 0.432087694 | 0.187655229 | PPT2; PPT2; PPT2; LOC100507547; | 37 | 6 |
| | | | | | | | | LOC100507547; LOC100507547; | | |
| | | | | | | | | LOC100507547; | | |
| | | | | | | | | PPT2-EGFL8 | | |
| cg23915527 | 7.13E−10 | 3.03E−04 | −0.244405927 | 0.244405927 | loss | 0.438410321 | 0.194004394 | | 37 | 1 |
| cg12565126 | 7.13E−10 | 3.03E−04 | −0.244383306 | 0.244383306 | loss | 0.803654653 | 0.559271347 | MFSD2B | 37 | 2 |
| cg22225479 | 7.13E−10 | 3.03E−04 | −0.244347363 | 0.244347368 | loss | 0.381545994 | 0.137198626 | | 37 | 17 |
| cg14085177 | 7.13E−10 | 3.03E−04 | −0.24433883 | 0.24433883 | loss | 0.805411583 | 0.561072753 | | 37 | 9 |
| cg08532057 | 7.13E−10 | 3.03E−04 | −0.244305766 | 0.244305766 | loss | 0.331310532 | 0.087004766 | NUPL1; NUPL1 | 37 | 13 |
| cg15012468 | 1.54E−09 | 6.54E−04 | −0.244272348 | 0.244272348 | loss | 0.797177965 | 0.552905618 | MFSD4 | 37 | 1 |
| cg26580869 | 2.16E−09 | 9.17E−04 | −0.24424914 | 0.24424914 | loss | 0.743812728 | 0.499563588 | HCN2 | 37 | 19 |
| cg26927427 | 1.10E−09 | 4.65E−04 | −0.244218658 | 0.244218658 | loss | 0.729103411 | 0.484884753 | KIFC3; KIFC3; KIFC3 | 37 | 16 |
| cg23573129 | 7.13E−10 | 3.03E−04 | −0.244214306 | 0.244214306 | loss | 0.485240742 | 0.241026435 | TBX1; TBX1; TBX1 | 37 | 22 |
| cg17395619 | 7.13E−10 | 3.03E−04 | −0.244195648 | 0.244195648 | loss | 0.760808489 | 0.516612841 | | 37 | 19 |
| cg06660015 | 7.13E−10 | 3.03E−04 | −0.244186345 | 0.244186345 | loss | 0.375791587 | 0.131605242 | SLC4A9; SLC4A9; SLC4A9; SLC4A9 | 37 | 5 |
| cg07647651 | 7.13E−10 | 3.03E−04 | −0.244185935 | 0.244185935 | loss | 0.458007611 | 0.213821676 | | 37 | 6 |
| cg19019345 | 1.54E−08 | 6.56E−03 | −0.244180482 | 0.244180482 | loss | 0.697753917 | 0.453573435 | ST5 | 37 | 11 |
| cg10963061 | 4.97E−09 | 2.11E−03 | −0.244179385 | 0.244179385 | loss | 0.564928785 | 0.3207494 | | 37 | 1 |
| cg18876602 | 5.84E−08 | 2.48E−02 | −0.244161319 | 0.244161319 | loss | 0.740009319 | 0.495848 | CAP2 | 37 | 6 |
| cg07140194 | 7.13E−10 | 3.03E−04 | −0.244151204 | 0.244151204 | loss | 0.841511787 | 0.597360582 | EMX1 | 37 | 2 |
| cg06464775 | 7.13E−10 | 3.03E−04 | −0.24411864 | 0.24411864 | loss | 0.463840434 | 0.219721794 | | 37 | 7 |
| cg09373148 | 9.23E−10 | 3.92E−04 | −0.244068942 | 0.244068942 | loss | 0.497050725 | 0.252981782 | ESYT3 | 37 | 3 |
| cg05324273 | 7.13E−10 | 3.03E−04 | −0.244067705 | 0.244067705 | loss | 0.875509017 | 0.631441312 | | 37 | 10 |
| cg21980100 | 1.10E−09 | 4.65E−04 | −0.24406045 | 0.24406045 | loss | 0.441683962 | 0.197623512 | CCDC144NL; CCDC144NL; | 37 | 17 |
| | | | | | | | | LOC440416 | | |
| cg07107628 | 5.84E−08 | 2.48E−02 | −0.244031786 | 0.244031786 | loss | 0.847794057 | 0.603762271 | | 37 | 6 |
| cg00071446 | 7.13E−10 | 3.03E−04 | −0.24402677 | 0.24402677 | loss | 0.925941611 | 0.681914841 | | 37 | 3 |
| cg06680906 | 7.13E−10 | 3.03E−04 | −0.244013515 | 0.244013515 | loss | 0.773528852 | 0.529515347 | NOX5; NOX5; NOX5; MIR548H4; | 37 | 15 |
| | | | | | | | | NOX5; NOX5 | | |
| cg06971773 | 7.77E−10 | 3.30E−04 | −0.244006216 | 0.244006216 | loss | 0.49102314 | 0.247016924 | TACR2 | 37 | 10 |
| cg01936707 | 7.77E−10 | 3.30E−04 | −0.243997248 | 0.243997248 | loss | 0.832674783 | 0.588677535 | DDR1; DDR1; DDR1; DDR1; | 37 | 6 |
| | | | | | | | | DDR1; DDR1 | | |
| cg22871908 | 7.84E−10 | 3.33E−04 | −0.243995865 | 0.243995865 | loss | 0.385017575 | 0.14102171 | | 37 | 1 |
| cg24912456 | 1.10E−09 | 4.65E−04 | −0.243990402 | 0.243990402 | loss | 0.872285696 | 0.628295294 | | 37 | 6 |
| cg06892912 | 7.13E−10 | 3.03E−04 | −0.243985953 | 0.243985953 | loss | 0.768254006 | 0.524268053 | RNF39; RNF39 | 37 | 6 |
| cg06767142 | 5.37E−08 | 2.28E−02 | −0.243925629 | 0.243925629 | loss | 0.944849542 | 0.700923913 | MACROD1 | 37 | 11 |
| cg01422370 | 1.10E−09 | 4.65E−04 | −0.243924759 | 0.243924759 | loss | 0.808787042 | 0.564862282 | | 37 | 2 |
| cg05589046 | 6.35E−09 | 2.70E−03 | −0.243922429 | 0.243922429 | loss | 0.9039235 | 0.660001071 | LINC00620 | 37 | 3 |
| cg21327235 | 6.89E−09 | 2.93E−03 | −0.243922332 | 0.243922332 | loss | 0.707612938 | 0.463690606 | DNAH2 | 37 | 17 |
| cg08687941 | 7.13E−10 | 3.03E−04 | −0.243913729 | 0.243913729 | loss | 0.756264406 | 0.512350676 | CNGB1; CNGB1 | 37 | 16 |
| cg21158317 | 7.13E−10 | 3.03E−04 | −0.243880151 | 0.243880151 | loss | 0.576863198 | 0.332983047 | RNF112 | 37 | 17 |
| cg02695062 | 7.13E−10 | 3.03E−04 | −0.24385523 | 0.24385523 | loss | 0.818401389 | 0.574546159 | DDR1; DDR1; DDR1 | 37 | 6 |
| cg16698038 | 7.13E−10 | 3.03E−04 | −0.243839321 | 0.243839321 | loss | 0.324348266 | 0.080508945 | | 37 | 5 |
| cg22379862 | 7.13E−10 | 3.03E−04 | −0.24383631 | 0.24383631 | loss | 0.892479834 | 0.648643524 | CACNA2D4 | 37 | 12 |
| cg26063721 | 9.23E−10 | 3.92E−04 | −0.24379749 | 0.24379749 | loss | 0.846222308 | 0.602424818 | PACSIN1; PACSIN1 | 37 | 6 |
| cg18041814 | 7.13E−10 | 3.03E−04 | −0.243784144 | 0.243784144 | loss | 0.838930891 | 0.595146747 | CPNE5 | 37 | 6 |
| cg24309555 | 7.13E−10 | 3.03E−04 | −0.243753104 | 0.243753104 | loss | 0.457300509 | 0.213547406 | APOB; APOB | 37 | 2 |
| cg12412902 | 1.01E−09 | 4.27E−04 | −0.243724162 | 0.243724162 | loss | 0.827029015 | 0.583304853 | PACSIN1; PACSIN1 | 37 | 6 |
| cg27565473 | 1.01E−09 | 4.27E−04 | −0.243722043 | 0.243722043 | loss | 0.821934713 | 0.578212671 | | 37 | 3 |
| cg05975881 | 7.13E−10 | 3.03E−04 | −0.24366575 | 0.24366575 | loss | 0.629908421 | 0.386242671 | ZSCAN10; ZSCAN10 | 37 | 16 |
| cg11931463 | 7.13E−10 | 3.03E−04 | −0.243649851 | 0.243649851 | loss | 0.528744787 | 0.285094935 | LINC00880; LINC00881 | 37 | 3 |
| cg10314139 | 7.77E−10 | 3.30E−04 | −0.243649453 | 0.243649453 | loss | 0.847678236 | 0.604028782 | COL13A1; COL13A1; COL13A1; | 37 | 10 |
| | | | | | | | | COL13A1; COL13A1; COL13A1 | | |
| cg12967164 | 9.23E−10 | 3.92E−04 | −0.243643311 | 0.243643311 | loss | 0.907683581 | 0.664040271 | ELFN1 | 37 | 7 |
| cg17288288 | 7.13E−10 | 3.03E−04 | −0.243641889 | 0.243641889 | loss | 0.70748563 | 0.463843741 | DLEU7; DLEU7-AS1 | 37 | 13 |
| cg02611848 | 7.13E−10 | 3.03E−04 | −0.243635954 | 0.243635954 | loss | 0.28798784 | 0.044351885 | M1AP; M1AP; M1AP; M1AP; | 37 | 2 |
| | | | | | | | | M1AP | | |
| cg05170706 | 2.12E−08 | 9.01E−03 | −0.24363229 | 0.24363229 | loss | 0.584742243 | 0.341109953 | | 37 | 13 |
| cg20543681 | 7.13E−10 | 3.03E−04 | −0.243626457 | 0.243626457 | loss | 0.487802034 | 0.244175576 | SCRT2 | 37 | 20 |
| cg01907837 | 4.57E−09 | 1.94E−03 | −0.243611757 | 0.243611757 | loss | 0.717398387 | 0.473786629 | RNF8; RNF8; RNF8 | 37 | 6 |
| cg17594670 | 1.12E−08 | 4.76E−03 | −0.24358266 | 0.24358266 | loss | 0.584015566 | 0.340432906 | | 37 | 1 |
| cg25364822 | 7.13E−10 | 3.03E−04 | −0.243581404 | 0.243581404 | loss | 0.748249751 | 0.504668347 | FNDC5; FNDC5; FNDC5 | 37 | 1 |
| cg21235746 | 2.35E−09 | 9.98E−04 | −0.243567607 | 0.243567607 | loss | 0.353790313 | 0.110222706 | LINC00654; LINC00654 | 37 | 20 |
| cg15022678 | 9.23E−10 | 3.92E−04 | −0.243560071 | 0.243560071 | loss | 0.849187947 | 0.605627876 | RADIL | 37 | 7 |
| cg19666541 | 1.12E−08 | 4.76E−03 | −0.243558929 | 0.243558929 | loss | 0.644061017 | 0.400502088 | LOC283710 | 37 | 15 |
| cg19419650 | 1.83E−09 | 7.75E−04 | −0.243534205 | 0.243534205 | loss | 0.513406928 | 0.269872724 | EVISL; EVISL | 37 | 19 |
| cg26651775 | 7.13E−10 | 3.03E−04 | −0.243516996 | 0.243516996 | loss | 0.781182313 | 0.537665318 | METRN | 37 | 16 |
| cg20645966 | 1.30E−09 | 5.52E−04 | −0.243514688 | 0.243514688 | loss | 0.7196797 | 0.476165012 | | 37 | 12 |
| cg05214460 | 7.13E−10 | 3.03E−04 | −0.243511899 | 0.243511899 | loss | 0.374569287 | 0.131057388 | ADD3; ADD3; ADD3; ADD3- | 37 | 10 |
| | | | | | | | | AS1 | | |
| cg10429573 | 1.19E−09 | 5.07E−04 | −0.243478025 | 0.243478025 | loss | 0.865461719 | 0.621983694 | | 37 | 2 |
| cg15138109 | 7.13E−10 | 3.03E−04 | −0.2434699 | 0.2434699 | loss | 0.6708792 | 0.4274093 | | 37 | 2 |
| cg13102294 | 7.13E−10 | 3.03E−04 | −0.243443327 | 0.243443327 | loss | 0.415566921 | 0.172123594 | PPT2; PPT2; PPT2; LOC100507547; | 37 | 6 |
| | | | | | | | | LOC100507547; LOC100507547; | | |
| | | | | | | | | LOC100507547; PPT2-EGFL8 | | |
| cg13882377 | 1.96E−08 | 8.33E−03 | −0.243389602 | 0.243389602 | loss | 0.715787591 | 0.472397988 | LHPP; LHPP | 37 | 10 |
| cg04726200 | 2.56E−09 | 1.09E−03 | −0.243306947 | 0.243306947 | loss | 0.591584047 | 0.3482771 | SLC22A18; SLC22A18 | 37 | 11 |
| cg26292895 | 1.91E−09 | 8.12E−04 | −0.243274541 | 0.243274541 | loss | 0.971050353 | 0.727775812 | RBM20 | 37 | 10 |
| cg04582871 | 7.13E−10 | 3.03E−04 | −0.243219495 | 0.243219495 | loss | 0.512332836 | 0.269113341 | MACROD1 | 37 | 11 |
| cg19981409 | 4.64E−08 | 1.97E−02 | −0.243188383 | 0.243188383 | loss | 0.45337473 | 0.210186347 | NOX4; NOX4; NOX4; NOX4 | 37 | 11 |
| cg24895155 | 3.97E−08 | 1.69E−02 | −0.24311435 | 0.24311435 | loss | 0.475877274 | 0.232762924 | | 37 | 8 |
| cg07028396 | 1.68E−09 | 7.12E−04 | −0.243109742 | 0.243109742 | loss | 0.491098925 | 0.247989182 | | 37 | 10 |
| cg03469862 | 9.23E−10 | 3.92E−04 | −0.243109231 | 0.243109231 | loss | 0.315066272 | 0.071957041 | LOC338694 | 37 | 11 |
| cg13347577 | 7.13E−10 | 3.03E−04 | −0.243109147 | 0.243109147 | loss | 0.689722694 | 0.446613547 | | 37 | 17 |
| cg15277677 | 3.40E−08 | 1.44E−02 | −0.243067386 | 0.243067386 | loss | 0.654410915 | 0.411343529 | NTM | 37 | 11 |
| cg13019868 | 1.30E−09 | 5.52E−04 | −0.243015428 | 0.243015428 | loss | 0.539885075 | 0.296869647 | | 37 | 11 |
| cg12187687 | 1.10E−09 | 4.65E−04 | −0.243010324 | 0.243010324 | loss | 0.627699742 | 0.384689418 | | 37 | 7 |
| cg09859240 | 7.13E−10 | 3.03E−04 | −0.242988101 | 0.242988101 | loss | 0.526521266 | 0.283533165 | MCMBP; MCMBP; MCMBP | 37 | 10 |
| cg18676967 | 7.84E−10 | 3.33E−04 | −0.242987948 | 0.242987948 | loss | 0.78183666 | 0.538848712 | | 37 | 2 |
| cg12468774 | 6.80E−08 | 2.89E−02 | −0.242923823 | 0.242923823 | loss | 0.455549764 | 0.212625941 | CCDC36; CCDC36 | 37 | 3 |
| cg27199697 | 8.47E−10 | 3.60E−04 | −0.242896985 | 0.242896985 | loss | 0.513433585 | 0.2705366 | DLX3 | 37 | 17 |
| cg14688272 | 7.13E−10 | 3.03E−04 | −0.24285562 | 0.24285562 | loss | 0.668626391 | 0.425770771 | FN3KRP; FN3KRP | 37 | 17 |
| cg13555278 | 7.13E−10 | 3.03E−04 | −0.242836963 | 0.242836963 | loss | 0.736464534 | 0.493627571 | EXTL1 | 37 | 1 |
| cg04264070 | 7.77E−10 | 3.30E−04 | −0.242804586 | 0.242804586 | loss | 0.442987068 | 0.200182482 | TMEM190 | 37 | 19 |
| cg23373640 | 7.77E−10 | 3.30E−04 | −0.242795338 | 0.242795338 | loss | 0.363482553 | 0.120687215 | | 37 | 14 |
| cg12222949 | 6.81E−08 | 2.89E−02 | −0.242794797 | 0.242794797 | loss | 0.369597762 | 0.126802965 | COLGALT2 | 37 | 1 |
| cg04352168 | 5.01E−08 | 2.13E−02 | −0.242788492 | 0.242788492 | loss | 0.783020409 | 0.540231918 | | 37 | 16 |
| cg27260867 | 1.03E−08 | 4.39E−03 | −0.242767507 | 0.242767507 | loss | 0.583448466 | 0.340680959 | | 37 | 13 |
| cg11314274 | 8.56E−10 | 3.63E−04 | −0.242745494 | 0.242745494 | loss | 0.985156588 | 0.742411094 | | 37 | 3 |
| cg18536118 | 1.43E−08 | 6.06E−03 | −0.242743625 | 0.242743625 | loss | 0.887474513 | 0.644730888 | | 37 | 5 |
| cg06621691 | 2.91E−08 | 1.23E−02 | −0.242736535 | 0.242736535 | loss | 0.830216764 | 0.587480229 | SDK1; SDK1 | 37 | 7 |
| cg10158997 | 4.29E−08 | 1.82E−02 | −0.242713387 | 0.242713387 | loss | 0.951539475 | 0.708826088 | VWA7 | 37 | 6 |
| cg26388730 | 7.13E−10 | 3.03E−04 | −0.242688813 | 0.242688813 | loss | 0.700159708 | 0.457470894 | NRXN2; NRXN2; NRXN2 | 37 | 11 |
| cg14331765 | 7.13E−10 | 3.03E−04 | −0.242686075 | 0.242686075 | loss | 0.832900787 | 0.590214712 | | 37 | 11 |
| cg18304280 | 7.13E−10 | 3.03E−04 | −0.242624438 | 0.242624438 | loss | 0.683094732 | 0.440470294 | | 37 | 14 |
| cg00873811 | 7.13E−10 | 3.03E−04 | −0.242605472 | 0.242605472 | loss | 0.887220072 | 0.6446146 | | 37 | 16 |
| cg14692950 | 1.22E−08 | 5.16E−03 | −0.242586831 | 0.242586831 | loss | 0.763697113 | 0.521110282 | DNAH2 | 37 | 17 |
| cg07603878 | 7.13E−10 | 3.03E−04 | −0.242581309 | 0.242581309 | loss | 0.913214379 | 0.670633071 | | 37 | 19 |
| cg12177762 | 1.99E−09 | 8.43E−04 | −0.242576784 | 0.242576784 | loss | 0.89187286 | 0.649296076 | C3orf56 | 37 | 3 |
| cg27206026 | 1.96E−08 | 8.33E−03 | −0.242535479 | 0.242535479 | loss | 0.578422791 | 0.335887312 | | 37 | 16 |
| cg11540997 | 7.13E−10 | 3.03E−04 | −0.242501336 | 0.242501336 | loss | 0.63062673 | 0.388125394 | DUOX2; DUOXA2 | 37 | 15 |
| cg03466522 | 7.13E−10 | 3.03E−04 | −0.24248694 | 0.24248694 | loss | 0.712399581 | 0.469912641 | FAM131C | 37 | 1 |
| cg08125503 | 8.47E−10 | 3.60E−04 | −0.24247875 | 0.24247875 | loss | 0.389341985 | 0.146863235 | PIGL; NCOR1 | 37 | 17 |
| cg24620905 | 1.10E−09 | 4.65E−04 | −0.242471936 | 0.242471936 | loss | 0.830490542 | 0.588018606 | MTMR11; MTMR11; MTMR11 | 37 | 1 |
| cg15033140 | 7.13E−10 | 3.03E−04 | −0.242390326 | 0.242390326 | loss | 0.769615338 | 0.527225012 | | 37 | 6 |
| cg06946880 | 7.13E−10 | 3.03E−04 | −0.242375492 | 0.242375492 | loss | 0.872441592 | 0.6300661 | ATP6V1B1 | 37 | 2 |
| cg09791504 | 8.47E−10 | 3.60E−04 | −0.242361942 | 0.242361942 | loss | 0.589027383 | 0.346665441 | RIIAD1 | 37 | 1 |
| cg07637741 | 5.39E−09 | 2.29E−03 | −0.242344367 | 0.242344367 | loss | 0.659777191 | 0.417432824 | SYTL3; SYTL3; SYTL3; SYTL3 | 37 | 6 |
| cg01695406 | 1.10E−09 | 4.65E−04 | −0.242335618 | 0.242335618 | loss | 0.760126247 | 0.517790629 | TMEM190 | 37 | 19 |
| cg05872387 | 7.13E−10 | 3.03E−04 | −0.242322427 | 0.242322427 | loss | 0.900112651 | 0.657790224 | GAL3ST2 | 37 | 2 |
| cg03985801 | 8.56E−10 | 3.63E−04 | −0.242321093 | 0.242321093 | loss | 0.668435446 | 0.426114353 | LGR6; LGR6; LGR6 | 37 | 1 |
| cg17347335 | 4.21E−09 | 1.79E−03 | −0.242320349 | 0.242320349 | loss | 0.781052808 | 0.538732459 | VSTM5 | 37 | 11 |
| cg15069551 | 7.13E−10 | 3.03E−04 | −0.242289962 | 0.242289962 | loss | 0.772603768 | 0.530313806 | LOC284798; LOC284798; LOC284798; | 37 | 20 |
| | | | | | | | | LOC284798 | | |
| cg12245717 | 7.13E−10 | 3.03E−04 | −0.242233751 | 0.242233751 | loss | 0.553831951 | 0.3115982 | LINC00261 | 37 | 20 |
| cg19863595 | 9.54E−09 | 4.05E−03 | −0.24221993 | 0.24221993 | loss | 0.816371147 | 0.574151218 | AMDHD1 | 37 | 12 |
| cg01817364 | 9.23E−10 | 3.92E−04 | −0.242202572 | 0.242202572 | loss | 0.859235919 | 0.617033347 | | 37 | 5 |
| cg09982942 | 2.48E−08 | 1.05E−02 | −0.242183501 | 0.242183501 | loss | 0.536828225 | 0.294644724 | FBLN7; FBLN7 | 37 | 2 |
| cg17635777 | 6.35E−09 | 2.70E−03 | −0.242170827 | 0.242170827 | loss | 0.648763374 | 0.406592547 | MROH5; MROH5; MROH5 | 37 | 8 |
| cg16675054 | 7.13E−10 | 3.03E−04 | −0.242110503 | 0.242110503 | loss | 0.988733232 | 0.746622729 | STK10 | 37 | 5 |
| cg09011891 | 7.13E−10 | 3.03E−04 | −0.242090261 | 0.242090261 | loss | 0.339597457 | 0.097507195 | C5orf66 | 37 | 5 |
| cg17808631 | 7.13E−10 | 3.03E−04 | −0.242086858 | 0.242086858 | loss | 0.676684411 | 0.434597553 | SNX31 | 37 | 8 |
| cg24172175 | 7.77E−10 | 3.30E−04 | −0.242077734 | 0.242077734 | loss | 0.875594392 | 0.633516659 | | 37 | 19 |
| cg12690246 | 7.13E−10 | 3.03E−04 | −0.242004056 | 0.242004056 | loss | 0.793920921 | 0.551916865 | AQP5 | 37 | 12 |
| cg04729574 | 7.13E−10 | 3.03E−04 | −0.241991499 | 0.241991499 | loss | 0.610738723 | 0.368747224 | ECE2; ECE2; ECE2; ECE2 | 37 | 3 |
| cg21124375 | 7.13E−10 | 3.03E−04 | −0.241974356 | 0.241974356 | loss | 0.406060898 | 0.164086542 | | 37 | 2 |
| cg17466082 | 3.67E−08 | 1.56E−02 | −0.241963799 | 0.241963799 | loss | 0.773434387 | 0.531470588 | | 37 | 2 |
| cg13047869 | 7.13E−10 | 3.03E−04 | −0.241961117 | 0.241961117 | loss | 0.975685206 | 0.733724088 | FANCD2OS; FANCD2OS; FANCD2OS; | 37 | 3 |
| | | | | | | | | FANCD2OS | | |
| cg25570495 | 2.48E−08 | 1.05E−02 | −0.241925425 | 0.241925425 | loss | 0.900061619 | 0.658136194 | SALL4; SALL4 | 37 | 20 |
| cg15808604 | 7.13E−10 | 3.03E−04 | −0.241912006 | 0.241912006 | loss | 0.891122206 | 0.6492102 | LOC283731 | 37 | 15 |
| cg23957311 | 7.13E−10 | 3.03E−04 | −0.241898273 | 0.241898273 | loss | 0.714151838 | 0.472253565 | ZIC4; ZIC4; ZIC4; ZIC4; Z1C4 | 37 | 3 |
| cg25215834 | 1.19E−09 | 5.07E−04 | −0.241886276 | 0.241886276 | loss | 0.5683208 | 0.326434524 | CARD14; CARD14; CARD14; | 37 | 17 |
| | | | | | | | | CARD14 | | |
| cg12559119 | 7.13E−10 | 3.03E−04 | −0.241873818 | 0.241873818 | loss | 0.43423093 | 0.192357112 | | 37 | 17 |
| cg02184697 | 7.13E−10 | 3.03E−04 | −0.24186336 | 0.24186336 | loss | 0.376234613 | 0.134371253 | KREMEN2; KREMEN2; KREMEN2; | 37 | 16 |
| | | | | | | | | KREMEN2 | | |
| cg06086012 | 4.64E−08 | 1.97E−02 | −0.241854443 | 0.241854443 | loss | 0.852711613 | 0.610857171 | CLDN11; CLDN11 | 37 | 3 |
| cg17024199 | 7.13E−10 | 3.03E−04 | −0.24183426 | 0.24183426 | loss | 0.514545908 | 0.272711647 | MGAT5B; MGAT5B; MGAT5B | 37 | 17 |
| cg23940612 | 7.13E−10 | 3.03E−04 | −0.241808897 | 0.241808897 | loss | 0.284333572 | 0.042524675 | RUFY4 | 37 | 2 |
| cg10243004 | 7.13E−10 | 3.03E−04 | −0.241806081 | 0.241806081 | loss | 0.87802204 | 0.636215959 | POLR2D | 37 | 2 |
| cg06737907 | 7.13E−10 | 3.03E−04 | −0.24180458 | 0.24180458 | loss | 0.808018721 | 0.566214141 | MEGF11 | 37 | 15 |
| cg26906273 | 7.13E−10 | 3.03E−04 | −0.24180165 | 0.24180165 | loss | 0.325622138 | 0.083820488 | | 37 | 1 |
| cg19642408 | 1.67E−08 | 7.10E−03 | −0.241798654 | 0.241798654 | loss | 0.761815419 | 0.520016765 | | 37 | 2 |
| cg16670359 | 7.13E−10 | 3.03E−04 | −0.241794477 | 0.241794477 | loss | 0.666586794 | 0.424792318 | | 37 | 8 |
| cg18156486 | 1.10E−09 | 4.65E−04 | −0.241794329 | 0.241794329 | loss | 0.398300228 | 0.156505899 | | 37 | 11 |
| cg21460582 | 7.13E−10 | 3.03E−04 | −0.241786055 | 0.241786055 | loss | 0.722324785 | 0.480538729 | CSPG4 | 37 | 15 |
| cg17600393 | 1.67E−08 | 7.10E−03 | −0.241784407 | 0.241784407 | loss | 0.69970173 | 0.457917324 | SNTG2 | 37 | 2 |
| cg16276982 | 8.47E−10 | 3.60E−04 | −0.241758218 | 0.241758218 | loss | 0.3115406 | 0.069782382 | | 37 | 15 |
| cg01602730 | 7.84E−10 | 3.33E−04 | −0.241747149 | 0.241747149 | loss | 0.327443617 | 0.085696468 | | 37 | 1 |
| cg02951357 | 7.13E−10 | 3.03E−04 | −0.241730843 | 0.241730843 | loss | 0.541659096 | 0.299928253 | LOC100131060; LOC100131060 | 37 | 1 |
| cg27664689 | 2.78E−09 | 1.18E−03 | −0.241722953 | 0.241722953 | loss | 0.555824194 | 0.314101241 | CACNB4; CACNB4; CACNB4; | 37 | 2 |
| | | | | | | | | CACNB4; CACNB4 | | |
| cg09501821 | 4.64E−08 | 1.97E−02 | −0.241658264 | 0.241658264 | loss | 0.582832528 | 0.341174265 | | 37 | 15 |
| cg10832281 | 1.83E−09 | 7.75E−04 | −0.241652523 | 0.241652523 | loss | 0.663040858 | 0.421388335 | PAMR1; PAMR1; PAMR1; PAMR1 | 37 | 11 |
| cg07513768 | 7.13E−10 | 3.03E−04 | −0.241644077 | 0.241644077 | loss | 0.317529513 | 0.075885436 | RPLP1; RPLP1 | 37 | 15 |
| cg04124909 | 7.13E−10 | 3.03E−04 | −0.241642517 | 0.241642517 | loss | 0.74014897 | 0.498506453 | ZNF648; ZNF648 | 37 | 1 |
| cg17379860 | 7.13E−10 | 3.03E−04 | −0.241635603 | 0.241635603 | loss | 0.422185468 | 0.180549865 | TMEM216; TMEM216; TMEM216 | 37 | 11 |
| cg24566261 | 7.13E−10 | 3.03E−04 | −0.241634012 | 0.241634012 | loss | 0.516230547 | 0.274596535 | DDR1; DDR1; DDR1 | 37 | 6 |
| cg06707910 | 2.56E−09 | 1.09E−03 | −0.241623506 | 0.241623506 | loss | 0.528964694 | 0.287341288 | CECR5; CECR5; CECR5- | 37 | 22 |
| | | | | | | | | AS1; CECR5-AS1 | | |
| cg18786782 | 7.13E−10 | 3.03E−04 | −0.241570568 | 0.241570568 | loss | 0.830724857 | 0.589154288 | | 37 | 3 |
| cg10894991 | 7.13E−10 | 3.03E−04 | −0.241539761 | 0.241539761 | loss | 0.754004955 | 0.512465194 | | 37 | 1 |
| cg22801949 | 7.77E−10 | 3.30E−04 | −0.241530482 | 0.241530482 | loss | 0.740754817 | 0.499224335 | IER5 | 37 | 1 |
| cg03453916 | 7.13E−10 | 3.03E−04 | −0.241509466 | 0.242509466 | loss | 0.363001223 | 0.121491757 | | 37 | 7 |
| cg04755995 | 7.13E−10 | 3.03E−04 | −0.241477635 | 0.241477635 | loss | 0.8806317 | 0.639154065 | | 37 | 7 |
| cg07704990 | 8.47E−10 | 3.60E−04 | −0.241470462 | 0.241470462 | loss | 0.905242079 | 0.663771618 | SLCO6A1; SLCO6A1 | 37 | 5 |
| cg09181015 | 9.23E−10 | 3.92E−04 | −0.241462402 | 0.241462402 | loss | 0.673695808 | 0.432233406 | | 37 | 5 |
| cg05875017 | 7.77E−10 | 3.30E−04 | −0.241449035 | 0.241449035 | loss | 0.336146885 | 0.094697849 | CHRND; CHRND; CHRND; CHRND | 37 | 2 |
| cg12635602 | 9.23E−10 | 3.92E−04 | −0.241445425 | 0.241445425 | loss | 0.823076402 | 0.581630976 | LOC283194 | 37 | 11 |
| cg01850889 | 7.13E−10 | 3.03E−04 | −0.241432379 | 0.241432379 | loss | 0.962002091 | 0.720569712 | | 37 | 2 |
| cg17718377 | 7.77E−10 | 3.30E−04 | −0.24141673 | 0.24141673 | loss | 0.700943936 | 0.459527206 | DTX1 | 37 | 12 |
| cg23226249 | 7.13E−10 | 3.03E−04 | −0.241415277 | 0.241415277 | loss | 0.915364677 | 0.6739494 | | 37 | 1 |
| cg21764262 | 7.77E−10 | 3.30E−04 | −0.241370538 | 0.241370538 | loss | 0.803545791 | 0.562175253 | | 37 | 6 |
| cg15142477 | 8.56E−10 | 3.63E−04 | −0.241360474 | 0.241360474 | loss | 0.42963461 | 0.188274135 | RNF165; RNF165; RNF165 | 37 | 18 |
| cg12924302 | 7.13E−10 | 3.03E−04 | −0.241358025 | 0.241358025 | loss | 0.948540219 | 0.707182194 | | 37 | 5 |
| cg07457305 | 7.13E−10 | 3.03E−04 | −0.241357284 | 0.241357284 | loss | 0.872655113 | 0.631297829 | | 37 | 2 |
| cg20615832 | 9.22E−08 | 3.91E−02 | −0.241345055 | 0.241345055 | loss | 0.433835443 | 0.192490388 | PF4V1 | 37 | 4 |
| cg15132710 | 7.13E−10 | 3.03E−04 | −0.241336531 | 0.241336531 | loss | 0.870667349 | 0.629330818 | LOXHD1 | 37 | 18 |
| cg02849695 | 1.10E−09 | 4.65E−04 | −0.241326893 | 0.241326893 | loss | 0.413938325 | 0.172611431 | CCDC19; CCDC19 | 37 | 1 |
| cg01569660 | 7.13E−10 | 3.03E−04 | −0.241273368 | 0.241273368 | loss | 0.784259368 | 0.542986 | KBTBD13 | 37 | 15 |
| cg12012524 | 7.13E−10 | 3.03E−04 | −0.241249943 | 0.241249943 | loss | 0.69763346 | 0.456383518 | CIRBP; CIRBP; CIRBP; CIRBP- | 37 | 19 |
| | | | | | | | | AS1 | | |
| cg06951677 | 1.99E−09 | 8.43E−04 | −0.241249857 | 0.241249857 | loss | 0.674667634 | 0.433417776 | | 37 | 1 |
| cg06888460 | 1.54E−09 | 6.54E−04 | −0.241249073 | 0.241249073 | loss | 0.720805432 | 0.479555359 | LDHAL6A; LDHAL6A | 37 | 11 |
| cg18480654 | 1.16E−07 | 4.91E−02 | −0.241219117 | 0.241219117 | loss | 0.859698128 | 0.618479012 | | 37 | 5 |
| cg09894276 | 1.83E−09 | 7.75E−04 | −0.241217102 | 0.241217102 | loss | 0.856471896 | 0.615254794 | WDR27; WDR27 | 37 | 6 |
| cg17598923 | 1.16E−07 | 4.91E−02 | −0.241206587 | 0.241206587 | loss | 0.570418628 | 0.329212041 | | 37 | 5 |
| cg02939598 | 7.84E−10 | 3.33E−04 | −0.241194312 | 0.241194312 | loss | 0.980698371 | 0.739504059 | | 37 | 10 |
| cg02635786 | 1.18E−08 | 5.01E−03 | −0.241178054 | 0.241178054 | loss | 0.932210665 | 0.691032612 | TDRD1 | 37 | 10 |
| cg19395706 | 7.13E−10 | 3.03E−04 | −0.241165996 | 0.241165996 | loss | 0.637695613 | 0.396529618 | NRXN2; NRXN2; NRXN2 | 37 | 11 |
| cg11389868 | 7.77E−10 | 3.30E−04 | −0.241153583 | 0.241153583 | loss | 0.75298973 | 0.511836147 | UGT3A1; UGT3A1; UGT3A1 | 37 | 5 |
| cg04760493 | 8.47E−10 | 3.60E−04 | −0.241130438 | 0.241130438 | loss | 0.682237826 | 0.441107388 | LARP1B; LARP1B; LARP1B; | 37 | 4 |
| | | | | | | | | LARP1B | | |
| cg00091633 | 7.84E−10 | 3.33E−04 | −0.241121666 | 0.241121666 | loss | 0.669234742 | 0.428113076 | FSTL1 | 37 | 3 |
| cg01830154 | 3.28E−09 | 1.39E−03 | −0.241117483 | 0.241117483 | loss | 0.411992636 | 0.170875153 | | 37 | 17 |
| cg04647654 | 7.77E−10 | 3.30E−04 | −0.241115505 | 0.241115505 | loss | 0.883598034 | 0.642482529 | | 37 | 12 |
| cg01057635 | 7.13E−10 | 3.03E−04 | −0.241108825 | 0.241108825 | loss | 0.910130208 | 0.669021382 | | 37 | 1 |
| cg21206816 | 1.83E−09 | 7.75E−04 | −0.241066616 | 0.241066616 | loss | 0.566027651 | 0.324961035 | SLC9C1; SLC9C1 | 37 | 3 |
| cg00073549 | 7.13E−10 | 3.03E−04 | −0.241049415 | 0.241049415 | loss | 0.701412179 | 0.460362765 | | 37 | 16 |
| cg08537845 | 7.13E−10 | 3.03E−04 | −0.241028266 | 0.241028266 | loss | 0.687689277 | 0.446661012 | | 37 | 5 |
| cg16707400 | 7.13E−10 | 3.03E−04 | −0.2410176 | 0.2410176 | loss | 0.776235442 | 0.535217841 | | 37 | 6 |
| cg21052814 | 3.40E−08 | 1.44E−02 | −0.240966791 | 0.240966791 | loss | 0.515071526 | 0.274104735 | RASGRF1; RASGRF1 | 37 | 15 |
| cg23429749 | 9.23E−10 | 3.92E−04 | −0.240909859 | 0.240909859 | loss | 0.774132142 | 0.533222282 | | 37 | 14 |
| cg20811236 | 7.77E−10 | 3.30E−04 | −0.240898438 | 0.240898438 | loss | 0.518086538 | 0.2771881 | | 37 | 18 |
| cg23183559 | 7.77E−10 | 3.30E−04 | −0.240895292 | 0.240895292 | loss | 0.907942898 | 0.667047606 | | 37 | 8 |
| cg18675610 | 2.78E−09 | 1.18E−03 | −0.240894906 | 0.240894906 | loss | 0.591469489 | 0.350574582 | ARHGAP12; ARHGAP12; ARHGAP12; | 37 | 10 |
| | | | | | | | | ARHGAP12; ARHGAP12 | | |
| cg05063520 | 2.69E−08 | 1.14E−02 | −0.240865606 | 0.240865606 | loss | 0.948433794 | 0.707568188 | | 37 | 8 |
| cg04452437 | 7.13E−10 | 3.03E−04 | −0.24084009 | 0.24084009 | loss | 0.950546449 | 0.709706359 | | 37 | 6 |
| cg10500653 | 3.87E−13 | 1.64E−07 | −0.240817785 | 0.240817785 | loss | 0.756535962 | 0.515718177 | RHBDL2; RHBDL2 | 37 | 1 |
| cg06081951 | 7.13E−10 | 3.03E−04 | −0.240816246 | 0.240816246 | loss | 0.439063081 | 0.198246835 | CALCA; CALCA; CALCA | 37 | 11 |
| cg02625222 | 3.88E−09 | 1.65E−03 | −0.240810076 | 0.240810076 | loss | 0.511888211 | 0.271078135 | CRB2 | 37 | 9 |
| cg07138585 | 7.13E−10 | 3.03E−04 | −0.240809542 | 0.240809542 | loss | 0.837919377 | 0.597109835 | | 37 | 2 |
| cg06881093 | 7.13E−10 | 3.03E−04 | −0.240789888 | 0.240789888 | loss | 0.359155119 | 0.118365231 | PAX8; PAX8; PAX8; PAX8 | 37 | 2 |
| cg02274869 | 7.77E−10 | 3.30E−04 | 0.240788067 | 0.240788067 | gain | 0.182786009 | 0.423574076 | ZNF833P | 37 | 19 |
| cg26955540 | 2.56E−09 | 1.09E−03 | −0.240758922 | 0.240758922 | loss | 0.680496551 | 0.439737629 | SEMA4B | 37 | 15 |
| cg09246479 | 7.13E−10 | 3.03E−04 | −0.240756146 | 0.240756146 | loss | 0.724794117 | 0.484037971 | UPB1; ADORA2A- | 37 | 22 |
| | | | | | | | | AS1; ADORA2A-AS1 | | |
| cg25042226 | 7.13E−10 | 3.03E−04 | −0.240725805 | 0.240725805 | loss | 0.848058781 | 0.607332976 | PAX8; PAX8; PAX8; PAX8; PAX8; | 37 | 2 |
| | | | | | | | | PAX8; PAX8; PAX8 | | |
| cg13630250 | 8.47E−10 | 3.60E−04 | −0.24067966 | 0.24067966 | loss | 0.804947977 | 0.564268318 | SYNJ2BP-COX16; SYNJ2BP- | 37 | 14 |
| | | | | | | | | COX16; SYNJ2BP- | | |
| | | | | | | | | COX16; COX16; COX16 | | |
| cg19225422 | 7.13E−10 | 3.03E−04 | −0.240657506 | 0.240657506 | loss | 0.6792249 | 0.438567394 | PDIA6; PDIA6; PDIA6 | 37 | 2 |
| cg06217323 | 1.68E−09 | 7.12E−04 | −0.240607807 | 0.240607807 | loss | 0.78496526 | 0.544357453 | | 37 | 3 |
| cg17385483 | 7.13E−10 | 3.03E−04 | −0.240598972 | 0.240598972 | loss | 0.525483972 | 0.284885 | | 37 | 12 |
| cg22392876 | 8.47E−10 | 3.60E−04 | −0.24055197 | 0.24055197 | loss | 0.873422206 | 0.632870235 | | 37 | 7 |
| cg05863637 | 7.84E−10 | 3.33E−04 | −0.240543824 | 0.240543824 | loss | 0.680549577 | 0.440005753 | | 37 | 12 |
| cg05902711 | 7.13E−10 | 3.03E−04 | −0.240503773 | 0.240503773 | loss | 0.683867138 | 0.443363365 | | 37 | 16 |
| cg20545694 | 1.19E−09 | 5.07E−04 | −0.240498628 | 0.240498628 | loss | 0.639014945 | 0.398516318 | | 37 | 8 |
| cg11946630 | 7.48E−09 | 3.18E−03 | −0.240444925 | 0.240444925 | loss | 0.780410466 | 0.539965541 | CRTAC1; CRTAC1 | 37 | 10 |
| cg24611264 | 7.13E−10 | 3.03E−04 | −0.240411643 | 0.240411643 | loss | 0.537836066 | 0.297424424 | TRIM29; TRIM29 | 37 | 11 |
| cg13476072 | 1.01E−09 | 4.27E−04 | −0.240411222 | 0.240411222 | loss | 0.762420751 | 0.522009529 | MTMR11; MTMR11; MTMR11 | 37 | 1 |
| cg24780796 | 1.19E−09 | 5.07E−04 | −0.240403012 | 0.240403012 | loss | 0.689148747 | 0.448745735 | SLC36A2 | 37 | 5 |
| cg01512532 | 7.13E−10 | 3.03E−04 | −0.240400662 | 0.240400662 | loss | 0.982968915 | 0.742568253 | FSCN2; FSCN2 | 37 | 17 |
| cg07776626 | 2.35E−09 | 9.98E−04 | −0.240399918 | 0.240399918 | loss | 0.84147223 | 0.601072312 | | 37 | 8 |
| cg25511813 | 9.23E−10 | 3.92E−04 | −0.240387252 | 0.240387252 | loss | 0.797190334 | 0.556803082 | DIRAS1 | 37 | 19 |
| cg01622718 | 1.54E−09 | 6.54E−04 | −0.240313957 | 0.240313957 | loss | 0.790856492 | 0.550542535 | PTPRN2; PTPRN2; PTPRN2 | 37 | 7 |
| cg27462791 | 7.13E−10 | 3.03E−04 | −0.240279444 | 0.240279444 | loss | 0.650884774 | 0.410605329 | SYT8 | 37 | 11 |
| cg23735482 | 6.89E−09 | 2.93E−03 | −0.240243204 | 0.240243204 | loss | 0.83734124 | 0.597098035 | MYO3A | 37 | 10 |
| cg16788656 | 7.13E−10 | 3.03E−04 | −0.240235099 | 0.240235099 | loss | 0.888530617 | 0.648295518 | | 37 | 16 |
| cg08203127 | 7.13E−10 | 3.03E−04 | −0.240233026 | 0.240233026 | loss | 0.628629238 | 0.388396212 | ARHGDIG; PDIA2 | 37 | 16 |
| cg14448104 | 3.88E−09 | 1.65E−03 | −0.240213489 | 0.240213489 | loss | 0.778132619 | 0.537919129 | MATN4; MATN4; MATN4 | 37 | 20 |
| cg01056004 | 1.19E−09 | 5.07E−04 | −0.240206759 | 0.240206759 | loss | 0.421616206 | 0.181409447 | ARHGAP19-SLIT1 | 37 | 10 |
| cg11316386 | 7.13E−10 | 3.03E−04 | −0.240203152 | 0.240203152 | loss | 0.525607423 | 0.285404271 | C1orf229; C1orf229 | 37 | 1 |
| cg15851904 | 1.89E−09 | 8.02E−04 | −0.240201052 | 0.240201052 | loss | 0.505063402 | 0.26486235 | | 37 | 12 |
| cg03918288 | 7.13E−10 | 3.03E−04 | −0.240188537 | 0.240188537 | loss | 0.59511056 | 0.354922024 | SPATC1; SPATC1 | 37 | 8 |
| cg09527118 | 1.42E−09 | 6.01E−04 | −0.24018543 | 0.24018543 | loss | 0.786722294 | 0.546536865 | | 37 | 11 |
| cg20806427 | 7.13E−10 | 3.03E−04 | −0.240184504 | 0.240184504 | loss | 0.443606264 | 0.203421761 | | 37 | 1 |
| cg14543641 | 7.13E−10 | 3.03E−04 | −0.240171982 | 0.240171982 | loss | 0.705953464 | 0.465781482 | | 37 | 14 |
| cg06421590 | 7.13E−10 | 3.03E−04 | −0.240162213 | 0.240162213 | loss | 0.352660653 | 0.11249844 | | 37 | 4 |
| cg07936030 | 7.13E−10 | 3.03E−04 | −0.24014702 | 0.24014702 | loss | 0.746432191 | 0.506285171 | SRCIN1 | 37 | 17 |
| cg07091678 | 1.12E−08 | 4.76E−03 | −0.240127937 | 0.240127937 | loss | 0.654700349 | 0.414572412 | CRTAC1; CRTAC1 | 37 | 10 |
| cg22152492 | 7.13E−10 | 3.03E−04 | −0.24011806 | 0.24011806 | loss | 0.851505272 | 0.611387212 | SCAND3 | 37 | 6 |
| cg26010879 | 1.30E−09 | 5.52E−04 | −0.240097106 | 0.240097106 | loss | 0.464119058 | 0.224021953 | | 37 | 7 |
| cg23281527 | 1.24E−08 | 5.26E−03 | −0.240088101 | 0.240088101 | loss | 0.91236867 | 0.672280569 | KLHDC7A | 37 | 1 |
| cg22138302 | 7.13E−10 | 3.03E−04 | −0.240087828 | 0.240087828 | loss | 0.597582087 | 0.357494259 | ABCG4; ABCG4 | 37 | 11 |
| cg26188543 | 7.48E−09 | 3.18E−03 | −0.240083492 | 0.240083492 | loss | 0.762903398 | 0.522819906 | | 37 | 3 |
| cg26076054 | 1.30E−09 | 5.52E−04 | −0.24007572 | 0.24007572 | loss | 0.806083196 | 0.566007476 | AHRR; AHRR | 37 | 5 |
| cg03350299 | 8.47E−10 | 3.60E−04 | −0.240072109 | 0.240072109 | loss | 0.423895626 | 0.183823518 | APOB; APOB | 37 | 2 |
| cg10855395 | 7.13E−10 | 3.03E−04 | −0.240061297 | 0.240061297 | loss | 0.360643419 | 0.120582122 | | 37 | 12 |
| cg20634798 | 1.22E−08 | 5.16E−03 | 0.240056031 | 0.240056031 | gain | 0.34379844 | 0.583854471 | LCE3A | 37 | 1 |
| cg24777351 | 1.96E−08 | 8.33E−03 | −0.240050746 | 0.240050746 | loss | 0.449891128 | 0.209840382 | SLC38A11; SLC38A11 | 37 | 2 |
| cg15602054 | 6.89E−09 | 2.93E−03 | −0.240005578 | 0.240005578 | loss | 0.64042846 | 0.400422882 | | 37 | 2 |
| cg24849736 | 1.54E−09 | 6.54E−04 | −0.240005152 | 0.240005152 | loss | 0.670264517 | 0.430259365 | ASCL5; ASCL5 | 37 | 1 |
| cg02975463 | 7.13E−10 | 3.03E−04 | −0.239989878 | 0.239989878 | loss | 0.447609855 | 0.207619976 | | 37 | 2 |
| cg01018110 | 7.48E−09 | 3.18E−03 | −0.239970768 | 0.239970768 | loss | 0.914149868 | 0.6741791 | | 37 | 5 |
| cg02087079 | 4.97E−09 | 2.11E−03 | −0.239956985 | 0.239956985 | loss | 0.588066443 | 0.348109459 | COL23A1 | 37 | 5 |
| cg06019590 | 1.54E−08 | 6.56E−03 | −0.239946998 | 0.239946998 | loss | 0.701048604 | 0.461101606 | SLC9A3; SLC9A3 | 37 | 5 |
| cg11523134 | 7.13E−10 | 3.03E−04 | −0.239944091 | 0.239944091 | loss | 0.872871668 | 0.632927576 | TP73; TP73; TP73; TP73; TP73; | 37 | 1 |
| | | | | | | | | TP73; TP73; TP73; TP73; | | |
| | | | | | | | | TP73; TP73; TP73 | | |
| cg11798891 | 7.13E−10 | 3.03E−04 | −0.23991798 | 0.23991798 | loss | 0.431760298 | 0.191842318 | TSNARE1 | 37 | 8 |
| cg24676346 | 7.77E−10 | 3.30E−04 | −0.239910975 | 0.239910975 | loss | 0.897358475 | 0.6574475 | | 37 | 6 |
| cg13977620 | 5.39E−09 | 2.29E−03 | −0.239884173 | 0.239884173 | loss | 0.813378279 | 0.573494106 | | 37 | 12 |
| cg21376799 | 7.13E−10 | 3.03E−04 | −0.239867457 | 0.239867457 | loss | 0.967754192 | 0.727886735 | | 37 | 6 |
| cg17881353 | 1.75E−09 | 7.44E−04 | −0.23983428 | 0.23983428 | loss | 0.722856198 | 0.483021918 | WNT9B | 37 | 17 |
| cg24006352 | 8.47E−10 | 3.60E−04 | −0.239813168 | 0.239813168 | loss | 0.851160957 | 0.611347788 | KCND3; KCND3 | 37 | 1 |
| cg15575875 | 7.13E−10 | 3.03E−04 | −0.239812872 | 0.239812872 | loss | 0.761676666 | 0.521863794 | | 37 | 1 |
| cg11821245 | 5.41E−08 | 2.30E−02 | −0.23980201 | 0.23980201 | loss | 0.69718284 | 0.457380829 | LDHC; LDHC | 37 | 11 |
| cg05265884 | 1.99E−09 | 8.43E−04 | −0.239781759 | 0.239781759 | loss | 0.455434106 | 0.215652347 | | 37 | 10 |
| cg23288838 | 2.12E−08 | 9.01E−03 | −0.239769111 | 0.239769111 | loss | 0.56639567 | 0.326626559 | | 37 | 8 |
| cg25351353 | 9.34E−10 | 3.97E−04 | −0.239707587 | 0.239707587 | loss | 0.948992152 | 0.709284565 | | 37 | 12 |
| cg03357547 | 7.13E−10 | 3.03E−04 | −0.239695753 | 0.239695753 | loss | 0.774401194 | 0.534705441 | SHANK1 | 37 | 19 |
| cg01919208 | 7.13E−10 | 3.03E−04 | −0.239693484 | 0.239693484 | loss | 0.402831298 | 0.163137814 | LAMB2; LAMB2 | 37 | 3 |
| cg16655765 | 4.96E−08 | 2.11E−02 | −0.23961761 | 0.23961761 | loss | 0.726193785 | 0.486576175 | METTL24 | 37 | 6 |
| cg08481491 | 7.13E−10 | 3.03E−04 | −0.23959792 | 0.23959792 | loss | 0.606173379 | 0.366575459 | ALDH1L1; ALDH1L1; ALDH1L1; | 37 | 3 |
| | | | | | | | | ALDH1L1; ALDH1L1- | | |
| | | | | | | | | AS2; ALDH1L1 | | |
| cg21415424 | 9.23E−10 | 3.92E−04 | −0.23957142 | 0.23957142 | loss | 0.690944038 | 0.451372618 | | 37 | 6 |
| cg14688299 | 2.56E−09 | 1.09E−03 | −0.239569903 | 0.239569903 | loss | 0.753711221 | 0.514141318 | | 37 | 13 |
| cg10138352 | 8.80E−09 | 3.74E−03 | −0.239550774 | 0.239550774 | loss | 0.579434162 | 0.339883388 | | 37 | 7 |
| cg04456916 | 7.34E−08 | 3.12E−02 | −0.239540082 | 0.239540082 | loss | 0.547220606 | 0.307680524 | | 37 | 5 |
| cg10061384 | 7.13E−10 | 3.03E−04 | −0.239528685 | 0.239528685 | loss | 0.794940179 | 0.555411494 | | 37 | 1 |
| cg14581129 | 1.30E−09 | 5.52E−04 | −0.239494551 | 0.239494551 | loss | 0.625035557 | 0.385541006 | | 37 | 12 |
| cg18358061 | 1.39E−08 | 5.90E−03 | −0.239458918 | 0.239458918 | loss | 0.893230188 | 0.653771271 | | 37 | 3 |
| cg05441133 | 1.01E−09 | 4.27E−04 | −0.239440426 | 0.239440426 | loss | 0.876219243 | 0.636778818 | GDF2; GDF2 | 37 | 10 |
| cg08688548 | 7.13E−10 | 3.03E−04 | −0.239440175 | 0.239440175 | loss | 0.838297187 | 0.598857012 | CPEB1; LOC283692 | 37 | 15 |
| cg20295248 | 2.35E−09 | 9.98E−04 | −0.239426374 | 0.239426374 | loss | 0.333596874 | 0.094170499 | LINC00654; LINC00654 | 37 | 20 |
| cg27430293 | 2.56E−09 | 1.09E−03 | −0.23941807 | 0.23941807 | loss | 0.507651258 | 0.268233188 | | 37 | 16 |
| cg01890726 | 1.68E−09 | 7.12E−04 | −0.239360699 | 0.239360699 | loss | 0.954276723 | 0.714916024 | GLB1L2 | 37 | 11 |
| cg08496033 | 7.13E−10 | 3.03E−04 | −0.239314577 | 0.239314577 | loss | 0.582775083 | 0.343460506 | | 37 | 8 |
| cg15794873 | 7.13E−10 | 3.03E−04 | −0.239292017 | 0.239292017 | loss | 0.500826411 | 0.261534394 | | 37 | 10 |
| cg02721176 | 8.47E−10 | 3.60E−04 | −0.239287335 | 0.239287335 | loss | 0.333039906 | 0.093752571 | CCDC172 | 37 | 10 |
| cg27190145 | 7.13E−10 | 3.03E−04 | −0.239285732 | 0.239285732 | loss | 0.823196832 | 0.5839111 | DAAM2; DAAM2 | 37 | 6 |
| cg02446487 | 7.13E−10 | 3.03E−04 | −0.239281696 | 0.239281696 | loss | 0.961440713 | 0.722159018 | NTN5; SEC1P | 37 | 19 |
| cg19594666 | 7.77E−10 | 3.30E−04 | −0.23927451 | 0.23927451 | loss | 0.439014851 | 0.199740341 | LEP | 37 | 7 |
| cg06872257 | 7.13E−10 | 3.03E−04 | −0.239271345 | 0.239271345 | loss | 0.413765598 | 0.174494253 | FGF20; FGF20 | 37 | 8 |
| cg07634019 | 7.77E−10 | 3.30E−04 | −0.239260409 | 0.239260409 | loss | 0.614298668 | 0.375038259 | PSD2 | 37 | 5 |
| cg22539356 | 3.88E−09 | 1.65E−03 | −0.239244068 | 0.239244068 | loss | 0.924575174 | 0.685331106 | | 37 | 15 |
| cg18955367 | 1.12E−08 | 4.76E−03 | −0.23920712 | 0.23920712 | loss | 0.701109932 | 0.461902812 | LMTK3 | 37 | 19 |
| cg15890794 | 7.77E−10 | 3.30E−04 | −0.239198593 | 0.239198593 | loss | 0.584495523 | 0.345296929 | CIDEA; CIDEA | 37 | 18 |
| cg08856033 | 2.16E−09 | 9.17E−04 | −0.239182719 | 0.239182719 | loss | 0.522420313 | 0.283237594 | | 37 | 2 |
| cg00653790 | 1.01E−09 | 4.27E−04 | −0.239153913 | 0.239153913 | loss | 0.602076672 | 0.362922759 | | 37 | 15 |
| cg25753631 | 3.88E−09 | 1.65E−03 | −0.239115382 | 0.239115382 | loss | 0.717161517 | 0.478046135 | HIST1H2APS1 | 37 | 6 |
| cg09015973 | 8.47E−10 | 3.60E−04 | −0.239100195 | 0.239100195 | loss | 0.310224117 | 0.071123922 | ARHGEF4; ARHGEF4 | 37 | 2 |
| cg02032841 | 7.84E−10 | 3.33E−04 | −0.239038633 | 0.239038633 | loss | 0.79433641 | 0.555297776 | | 37 | 3 |
| cg11001769 | 2.16E−09 | 9.17E−04 | −0.239025305 | 0.239025305 | loss | 0.857927611 | 0.618902306 | TMEM30B; TMEM30B | 37 | 14 |
| cg11350504 | 2.78E−09 | 1.18E−03 | −0.239020406 | 0.239020406 | loss | 0.867479642 | 0.628459235 | | 37 | 13 |
| cg00161556 | 3.67E−08 | 1.56E−02 | −0.239017431 | 0.239017431 | loss | 0.561906766 | 0.322889335 | ZDHHC13; ZDHHC13 | 37 | 11 |
| cg22126965 | 2.91E−08 | 1.23E−02 | −0.238959218 | 0.238959218 | loss | 0.667035694 | 0.428076476 | COX19 | 37 | 7 |
| cg03400060 | 7.13E−10 | 3.03E−04 | −0.238937467 | 0.238937467 | loss | 0.496680879 | 0.257743412 | BHMT2; DMGDH; BHMT2; | 37 | 5 |
| | | | | | | | | DMGDH; DMGDH | | |
| cg09813050 | 1.01E−09 | 4.27E−04 | −0.238925128 | 0.238925128 | loss | 0.385442926 | 0.146517798 | | 37 | 11 |
| cg04675946 | 9.23E−10 | 3.92E−04 | −0.238921769 | 0.238921769 | loss | 0.652594551 | 0.413672782 | | 37 | 8 |
| cg09656511 | 7.84E−10 | 3.33E−04 | −0.238882869 | 0.238882869 | loss | 0.971177192 | 0.732294324 | CCKBR | 37 | 11 |
| cg23824713 | 1.01E−09 | 4.27E−04 | −0.238855589 | 0.238855589 | loss | 0.797303325 | 0.558453235 | SERP1NB5 | 37 | 18 |
| cg01786585 | 7.13E−10 | 3.03E−04 | −0.238842519 | 0.238842519 | loss | 0.895321866 | 0.656479347 | | 37 | 6 |
| cg23083049 | 9.23E−10 | 3.92E−04 | −0.238799259 | 0.238799259 | loss | 0.890299836 | 0.651500576 | KIRREL3; KIRREL3 | 37 | 11 |
| cg13677022 | 7.13E−10 | 3.03E−04 | −0.238789185 | 0.238789185 | loss | 0.841489291 | 0.602700106 | ARID3C | 37 | 9 |
| cg11697038 | 7.13E−10 | 3.03E−04 | −0.238773634 | 0.238773634 | loss | 0.662394887 | 0.423621253 | AHDC1 | 37 | 1 |
| cg02959725 | 3.88E−09 | 1.65E−03 | −0.23875906 | 0.23875906 | loss | 0.839472177 | 0.600713118 | | 37 | 3 |
| cg17394530 | 7.13E−10 | 3.03E−04 | −0.238741723 | 0.238741723 | loss | 0.585488258 | 0.346746535 | | 37 | 6 |
| cg07292311 | 8.47E−10 | 3.60E−04 | −0.238740399 | 0.238740399 | loss | 0.706660234 | 0.467919835 | ZNF664-FAM101A | 37 | 12 |
| cg20132698 | 1.83E−09 | 7.75E−04 | −0.238739161 | 0.238739161 | loss | 0.727217785 | 0.488478624 | | 37 | 11 |
| cg01786048 | 2.69E−08 | 1.14E−02 | −0.238729697 | 0.238729697 | loss | 0.391285219 | 0.152555522 | ESPNL | 37 | 2 |
| cg11684897 | 8.47E−10 | 3.60E−04 | −0.238709684 | 0.238709684 | loss | 0.530498355 | 0.291788671 | | 37 | 17 |
| cg17481703 | 3.67E−08 | 1.56E−02 | −0.238683369 | 0.238683369 | loss | 0.72G49364 | 0.487810271 | C13orf45 | 37 | 13 |
| cg23635560 | 7.13E−10 | 3.03E−04 | −0.238681905 | 0.238681905 | loss | 0.674869728 | 0.436187824 | | 37 | 2 |
| cg13337662 | 7.77E−10 | 3.30E−04 | −0.238653066 | 0.238653066 | loss | 0.441116266 | 0.2024632 | ATP12A; ATP12A | 37 | 13 |
| cg03405128 | 7.13E−10 | 3.03E−04 | −0.238652665 | 0.238652665 | loss | 0.49997393 | 0.261321265 | | 37 | 4 |
| cg13783444 | 7.13E−10 | 3.03E−04 | −0.238636512 | 0.238636512 | loss | 0.407572436 | 0.168935924 | 2C2HC1C; ZC2HC1C | 37 | 14 |
| cg20354882 | 1.81E−08 | 7.69E−03 | −0.238611959 | 0.238611959 | loss | 0.655877306 | 0.417265347 | AMDHD1 | 37 | 12 |
| cg08834436 | 1.12E−08 | 4.76E−03 | −0.238609518 | 0.238609518 | loss | 0.473532789 | 0.234923271 | | 37 | 22 |
| cg13708016 | 1.10E−09 | 4.65E−04 | −0.238596595 | 0.238596595 | loss | 0.465871989 | 0.227275394 | | 37 | 3 |
| cg16988970 | 7.13E−10 | 3.03E−04 | −0.238574881 | 0.238574881 | loss | 0.548471775 | 0.309896894 | ASB16 | 37 | 17 |
| cg00188627 | 7.13E−10 | 3.03E−04 | −0.238561659 | 0.238561659 | loss | 0.397765828 | 0.159204169 | SCHIP1; SCHIP1; SCHIP1; IQCJ- | 37 | 3 |
| | | | | | | | | SCHIP1; IQCJ- | | |
| | | | | | | | | SCHIP1; SCHIP1 | | |
| cg01497154 | 7.13E−10 | 3.03E−04 | −0.238510632 | 0.238510632 | loss | 0.410425926 | 0.171915294 | ADAM33; ADAM33; ADAM33 | 37 | 20 |
| cg22588640 | 1.73E−09 | 7.34E−04 | −0.238491882 | 0.238491882 | loss | 0.429855194 | 0.191363313 | KCNIP3 | 37 | 2 |
| cg20724680 | 2.78E−09 | 1.18E−03 | −0.238463649 | 0.238463649 | loss | 0.702339108 | 0.453875459 | LMTK3 | 37 | 19 |
| cg25486824 | 1.12E−08 | 4.76E−03 | −0.238443169 | 0.238443169 | loss | 0.871165628 | 0.632722459 | SPON1 | 37 | 11 |
| cg11316100 | 7.13E−10 | 3.03E−04 | −0.238425595 | 0.238425595 | loss | 0.426354372 | 0.187928776 | C20orf24; TGIF2- | 37 | 20 |
| | | | | | | | | C20orf24; C20orf24; C20orf24; | | |
| | | | | | | | | C20orf24 | | |
| cg16592832 | 8.47E−10 | 3.60E−04 | −0.238409903 | 0.238409903 | loss | 0.478741421 | 0.240331518 | UPB1; ADORA2A- | 37 | 22 |
| | | | | | | | | AS1; ADORA2A-AS1 | | |
| cg02637101 | 1.99E−09 | 8.43E−04 | −0.238371703 | 0.238371703 | loss | 0.654732232 | 0.416360529 | PGPEP1L; PGPEP1L; PGPEP1L | 37 | 15 |
| cg26682922 | 7.13E−10 | 3.03E−04 | −0.23837047 | 0.23837047 | loss | 0.610098217 | 0.371727747 | | 37 | 1 |
| cg13982318 | 7.13E−10 | 3.03E−04 | −0.238365433 | 0.233365433 | loss | 0.892739321 | 0.654373888 | APBA1 | 37 | 9 |
| cg26333393 | 8.47E−10 | 3.60E−04 | −0.238351026 | 0.238351026 | loss | 0.723305996 | 0.484954971 | CPXM2 | 37 | 10 |
| cg03731646 | 1.68E−09 | 7.12E−04 | −0.238342982 | 0.238342982 | loss | 0.64740267 | 0.409059688 | | 37 | 18 |
| cg16734433 | 3.14E−08 | 1.33E−02 | −0.238331941 | 0.238331941 | loss | 0.916169094 | 0.677837153 | | 37 | 10 |
| cg17035539 | 2.35E−09 | 9.98E−04 | −0.238326002 | 0.238326002 | loss | 0.615061938 | 0.376735935 | | 37 | 1 |
| cg18564464 | 8.11E−09 | 3.44E−03 | −0.238300325 | 0.238300325 | loss | 0.699677955 | 0.461377629 | | 37 | 1 |
| cg01706515 | 1.12E−08 | 4.76E−03 | −0.238287837 | 0.238287837 | loss | 0.735742302 | 0.497454465 | CRISP2; CRISP2; CRISP2; CRISP2; | 37 | 6 |
| | | | | | | | | CRISP2; CRISP2 | | |
| cg24332389 | 7.77E−10 | 3.30E−04 | −0.238286759 | 0.238286759 | loss | 0.871858153 | 0.633571394 | PLEKHG5; PLEKHG5; PLEKHG5 | 37 | 1 |
| cg27113419 | 3.02E−09 | 1.28E−03 | −0.238255104 | 0.238255104 | loss | 0.361434406 | 0.123179302 | NDRG4; NDRG4; NDRG4; NDRG4; | 37 | 16 |
| | | | | | | | | NDRG4; NDRG4; NDRG4; | | |
| | | | | | | | | NDRG4 | | |
| cg23490796 | 7.13E−10 | 3.03E−04 | −0.238251859 | 0.238251859 | loss | 0.584323883 | 0.346072024 | DES | 37 | 2 |
| cg00126498 | 2.16E−09 | 9.17E−04 | −0.23823361 | 0.23823361 | loss | 0.493998134 | 0.255764524 | ULBP1 | 37 | 6 |
| cg04189591 | 7.13E−10 | 3.03E−04 | −0.238233007 | 0.238233007 | loss | 0.515575519 | 0.277342512 | SHISA8 | 37 | 22 |
| cg09477019 | 7.13E−10 | 3.03E−04 | −0.238217252 | 0.238217252 | loss | 0.398940134 | 0.160722882 | | 37 | 16 |
| cg15209808 | 7.77E−10 | 3.30E−04 | −0.238211667 | 0.238211667 | loss | 0.383276204 | 0.145054536 | HORMAD2 | 37 | 22 |
| cg11599830 | 7.13E−10 | 3.03E−04 | −0.238211225 | 0.238211225 | loss | 0.451148366 | 0.212937141 | LMBR1L | 37 | 12 |
| cg00093178 | 5.85E−09 | 2.49E−03 | −0.238200885 | 0.238200885 | loss | 0.778822926 | 0.540622041 | | 37 | 13 |
| cg16406611 | 1.81E−08 | 7.69E−03 | −0.238157843 | 0.238157843 | loss | 0.627090196 | 0.388932353 | IFITM5 | 37 | 11 |
| cg12946961 | 1.19E−09 | 5.07E−04 | −0.238149103 | 0.238149103 | loss | 0.911883968 | 0.673734865 | | 37 | 5 |
| cg23175533 | 1.16E−07 | 4.91E−02 | −0.238139571 | 0.238139571 | loss | 0.726053042 | 0.487913471 | KIAA1467 | 37 | 12 |
| cg10938046 | 7.13E−10 | 3.03E−04 | −0.238131077 | 0.238131077 | loss | 0.873359183 | 0.635228106 | C6orf223; C6orf223 | 37 | 6 |
| cg17962756 | 2.12E−08 | 9.01E−03 | −0.23808803 | 0.23808803 | loss | 0.575377089 | 0.337289059 | | 37 | 5 |
| cg04733365 | 7.77E−10 | 3.30E−04 | −0.238040856 | 0.238040856 | loss | 0.709098332 | 0.471057476 | | 37 | 11 |
| cg05309280 | 8.47E−10 | 3.60E−04 | −0.238040509 | 0.238040509 | loss | 0.770096432 | 0.532055924 | GORASP2; GORASP2 | 37 | 2 |
| cg26332285 | 7.13E−10 | 3.03E−04 | −0.238000682 | 0.238000682 | loss | 0.7646623 | 0.526661618 | GPR123 | 37 | 10 |
| cg16524232 | 7.13E−10 | 3.03E−04 | −0.237998163 | 0.237998163 | loss | 0.72952254 | 0.491524376 | | 37 | 16 |
| cg11241151 | 1.42E−09 | 6.01E−04 | −0.23799682 | 0.23799682 | loss | 0.800681102 | 0.562684282 | | 37 | 2 |
| cg24735129 | 7.77E−10 | 3.30E−04 | −0.237974694 | 0.237974694 | loss | 0.537843306 | 0.299868612 | | 37 | 11 |
| cg10350350 | 2.69E−08 | 1.14E−02 | −0.237961212 | 0.237961212 | loss | 0.732829842 | 0.494865629 | | 37 | 11 |
| cg26211724 | 1.99E−09 | 8.43E−04 | −0.237946899 | 0.237946899 | loss | 0.719336164 | 0.481389265 | | 37 | 18 |
| cg23669855 | 9.23E−10 | 3.92E−04 | −0.23793345 | 0.23793345 | loss | 0.929901462 | 0.691968012 | LYSMD4; LYSMD4; LYSMD4; | 37 | 15 |
| | | | | | | | | LYSMD4; LYSMD4; LYSMD4; | | |
| | | | | | | | | LYSMD4 | | |
| cg13736131 | 1.19E−09 | 5.07E−04 | −0.237921023 | 0.237921023 | loss | 0.541493417 | 0.303572394 | ST5; ST5; ST5; ST5 | 37 | 11 |
| cg13924715 | 8.55E−08 | 3.63E−02 | −0.237888027 | 0.237888027 | loss | 0.92067851S | 0.682790488 | | 37 | 11 |
| cg06626847 | 7.13E−10 | 3.03E−04 | −0.237886684 | 0.237886684 | loss | 0.391770443 | 0.153883759 | | 37 | 1 |
| cg23604683 | 7.13E−10 | 3.03E−04 | −0.237788513 | 0.237788513 | loss | 0.76716416 | 0.529375647 | | 37 | 7 |
| cg05404005 | 7.13E−10 | 3.03E−04 | −0.237743153 | 0.237743153 | loss | 0.568021642 | 0.330278488 | | 37 | 11 |
| cg19628603 | 1.42E−09 | 6.01E−04 | −0.237713411 | 0.237713411 | loss | 0.561873617 | 0.324160206 | | 37 | 6 |
| C616598508 | 1.01E−09 | 4.27E−04 | −0.237702474 | 0.237702474 | loss | 0.452092757 | 0.214390282 | | 37 | 16 |
| cg14130459 | 1.30E−09 | 5.52E−04 | −0.23769835 | 0.23769835 | loss | 0.530465321 | 0.292766971 | SLC15A3; SLC15A3 | 37 | 11 |
| cg22496437 | 7.13E−10 | 3.03E−04 | −0.237679677 | 0.237679677 | loss | 0.601537889 | 0.363858212 | ARHGEF10L | 37 | 1 |
| cg08266168 | 7.13E−10 | 3.03E−04 | −0.237678853 | 0.237678853 | loss | 0.783485694 | 0.545806841 | F7; F7; F7; F7 | 37 | 13 |
| cg16306259 | 2.30E−08 | 9.75E−03 | −0.237641054 | 0.237641054 | loss | 0.890983466 | 0.653342412 | | 37 | 6 |
| cg13443627 | 2.16E−09 | 9.17E−04 | −0.237629604 | 0.237629604 | loss | 0.3436352 | 0.106005596 | TACSTD2; TACSTD2 | 37 | 1 |
| cg07337544 | 7.84E−10 | 3.33E−04 | −0.237613713 | 0.237613713 | loss | 0.765490025 | 0.527876312 | KCNIP3; KCNIP3 | 37 | 2 |
| cg16555466 | 1.19E−09 | 5.07E−04 | −0.237611931 | 0.237611931 | loss | 0.543118666 | 0.305506735 | | 37 | 14 |
| cg13810327 | 7.13E−10 | 3.03E−04 | −0.237609743 | 0.237609743 | loss | 0.902104413 | 0.664494671 | C9orf171; C9orf171 | 37 | 9 |
| cg26740318 | 1.68E−09 | 7.12E−04 | −0.237563477 | 0.237563477 | loss | 0.697787436 | 0.460223959 | LDHAL6A; LDHAL6A | 37 | 11 |
| cg19094438 | 7.13E−10 | 3.03E−04 | −0.237556508 | 0.237556508 | loss | 0.572295255 | 0.334738747 | BCAR1; BCAR1; BCAR1; BCAR1; | 37 | 16 |
| | | | | | | | | BCAR1; BCAR1; BCAR1 | | |
| cg18449739 | 1.42E−09 | 6.01E−04 | −0.237553504 | 0.237553504 | loss | 0.726098092 | 0.488544588 | DTX1 | 37 | 12 |
| cg09777637 | 3.14E−08 | 1.33E−02 | −0.237549015 | 0.237549015 | loss | 0.622237109 | 0.384688094 | TMEM229A | 37 | 7 |
| cg17604312 | 7.13E−10 | 3.03E−04 | −0.237537785 | 0.237537785 | loss | 0.686617232 | 0.449079447 | DDR1; DDR1 | 37 | 6 |
| cg04518186 | 6.35E−09 | 2.70E−03 | −0.237471437 | 0.237471437 | loss | 0.768961496 | 0.531490059 | AQP11 | 37 | 11 |
| cg14807565 | 7.13E−10 | 3.03E−04 | −0.237450908 | 0.237450908 | loss | 0.524144102 | 0.286693194 | | 37 | 10 |
| cg20905088 | 5.41E−08 | 2.30E−02 | −0.237431582 | 0.237431582 | loss | 0.835945306 | 0.598513724 | PTHLH; PTHLH; PTHLH; PTHLH | 37 | 12 |
| cg18621852 | 7.13E−10 | 3.03E−04 | −0.23742772 | 0.23742772 | loss | 0.885958296 | 0.648530576 | FANCD2OS; FANCD2OS; FANCD2OS; | 37 | 3 |
| | | | | | | | | FANCD2OS | | |
| cg03079132 | 7.13E−10 | 3.03E−04 | −0.237388993 | 0.237388993 | loss | 0.480610475 | 0.243221482 | RHBDL1; RHBDL1 | 37 | 16 |
| cg22625519 | 7.13E−10 | 3.03E−04 | −0.237383458 | 0.237383458 | loss | 0.659342128 | 0.421958671 | | 37 | 6 |
| cg13099429 | 8.47E−10 | 3.60E−04 | −0.237377134 | 0.237377134 | loss | 0.413941545 | 0.176564412 | SYNJ2BP-COX16; SYNJ2BP- | 37 | 14 |
| | | | | | | | | COX16; SYNJ2BP- | | |
| | | | | | | | | COX16; COX16; COX16 | | |
| cg24964694 | 1.19E−09 | 5.07E−04 | −0.237376378 | 0.237376378 | loss | 0.375103962 | 0.137727584 | | 37 | 6 |
| cg08276171 | 7.13E−10 | 3.03E−04 | −0.237367231 | 0.237367231 | loss | 0.573016972 | 0.33S649741 | | 37 | 14 |
| cg09122655 | 7.13E−10 | 3.03E−04 | −0.237351856 | 0.237351856 | loss | 0.747698362 | 0.510346506 | NPFFR1 | 37 | 10 |
| cg27455017 | 7.13E−10 | 3.03E−04 | −0.237324321 | 0.237324321 | loss | 0.454950962 | 0.217626641 | RARRES2 | 37 | 7 |
| cg24940230 | 9.23E−10 | 3.92E−04 | −0.237297279 | 0.237297279 | loss | 0.723875874 | 0.486578594 | | 37 | 11 |
| cg14070755 | 7.13E−10 | 3.03E−04 | −0.23729597 | 0.23729597 | loss | 0.583498553 | 0.346202582 | MLPH; MLPH; MLPH; MLPH; | 37 | 2 |
| | | | | | | | | MLPH | | |
| cg09113440 | 8.11E−09 | 3.44E−03 | −0.237271634 | 0.237271634 | loss | 0.812856581 | 0.575584947 | | 37 | 10 |
| cg01294283 | 7.13E−10 | 3.03E−04 | −0.237267925 | 0.237267925 | loss | 0.888330213 | 0.651062288 | | 37 | 2 |
| cg24848998 | 1.99E−09 | 8.43E−04 | −0.237217104 | 0.237217104 | loss | 0.805170492 | 0.567953388 | GRIP2 | 37 | 3 |
| cg05161119 | 7.77E−10 | 3.30E−04 | −0.237176024 | 0.237176024 | loss | 0.685046189 | 0.447870165 | CPLX1 | 37 | 4 |
| cg16292132 | 3.14E−08 | 1.33E−02 | −0.237174871 | 0.237174871 | loss | 0.719453 | 0.482288129 | NHSL1 | 37 | 6 |
| cg06906952 | 9.54E−09 | 4.05E−03 | −0.237125921 | 0.237125921 | loss | 0.942417057 | 0.705291135 | | 37 | 1 |
| cg16449012 | 3.97E−08 | 1.69E−02 | −0.237117058 | 0.237117058 | loss | 0.727773423 | 0.490656365 | FAM184B | 37 | 4 |
| cg03717239 | 7.13E−10 | 3.03E−04 | −0.237093229 | 0.237093229 | loss | 0.563127706 | 0.326034476 | | 37 | 8 |
| cg15146462 | 7.92E−08 | 3.36E−02 | −0.237087668 | 0.237087668 | loss | 0.720074521 | 0.482986853 | | 37 | 11 |
| cg23557906 | 7.13E−10 | 3.03E−04 | −0.237078192 | 0.237078192 | loss | 0.946794104 | 0.709715912 | | 37 | 2 |
| cg05225593 | 7.13E−10 | 3.03E−04 | −0.237077312 | 0.237077312 | loss | 0.903782106 | 0.666704794 | | 37 | 16 |
| cg25478299 | 8.80E−09 | 3.74E−03 | −0.237066897 | 0.237066897 | loss | 0.804439038 | 0.567372141 | | 37 | 11 |
| cg11402505 | 7.13E−10 | 3.03E−04 | −0.237041233 | 0.237041233 | loss | 0.30236544 | 0.065324207 | FBXO27 | 37 | 19 |
| cg01591830 | 7.77E−10 | 3.30E−04 | −0.237025216 | 0.237025216 | loss | 0.493989604 | 0.256964388 | | 37 | 16 |
| cg08681856 | 9.23E−10 | 3.92E−04 | −0.237006576 | 0.237006576 | loss | 0.666077864 | 0.429071288 | KLK4 | 37 | 19 |
| cg05100067 | 9.23E−10 | 3.92E−04 | −0.236983148 | 0.236983148 | loss | 0.713914983 | 0.476931835 | MYT1L | 37 | 2 |
| cg01256227 | 1.83E−09 | 7.75E−04 | −0.236978947 | 0.236978947 | loss | 0.500565247 | 0.2635863 | | 37 | 6 |
| cg05697249 | 7.77E−10 | 3.30E−04 | −0.236965226 | 0.236965226 | loss | 0.703327838 | 0.466362612 | C11orf52; HSPB2- | 37 | 11 |
| | | | | | | | | C11orf52 | | |
| cg12894629 | 7.13E−10 | 3.03E−04 | −0.2369556 | 0.2369556 | loss | 0.820416858 | 0.583461259 | SLC51A | 37 | 3 |
| cg19642433 | 7.13E−10 | 3.03E−04 | −0.236951613 | 0.236951613 | loss | 0.880137019 | 0.643185406 | C12orf56; C12orf56 | 37 | 12 |
| cg03336167 | 2.16E−09 | 9.17E−04 | −0.236951431 | 0.236951431 | loss | 0.362583532 | 0.125632101 | SLC22A18; SLC22A18 | 37 | 11 |
| cg27255656 | 1.32E−08 | 5.59E−03 | −0.236948323 | 0.236948323 | loss | 0.635008823 | 0.3980605 | CSMD1 | 37 | 8 |
| cg06649280 | 7.13E−10 | 3.03E−04 | −0.236941875 | 0.236941875 | loss | 0.517203287 | 0.280261412 | MYOM3; MTOM3 | 37 | 1 |
| cg09372728 | 9.23E−10 | 3.92E−04 | −0.236915405 | 0.236915405 | loss | 0.81536107 | 0.578445665 | MT3 | 37 | 16 |
| cg12976582 | 7.13E−10 | 3.03E−04 | −0.23690851 | 0.23690851 | loss | 0.372304558 | 0.135396049 | HYAL2; HYAL2 | 37 | 3 |
| cg00228891 | 7.13E−10 | 3.03E−04 | −0.236883715 | 0.236883715 | loss | 0.685046209 | 0.448162494 | CR1L | 37 | 1 |
| cg09635954 | 8.47E−10 | 3.60E−04 | −0.23687516 | 0.23687516 | loss | 0.573304272 | 0.336429112 | PRR15 | 37 | 7 |
| cg12554483 | 1.01E−09 | 4.27E−04 | −0.236868951 | 0.236868951 | loss | 0.363010958 | 0.126142007 | VENTX | 37 | 10 |
| cg15452204 | 7.13E−10 | 3.03E−04 | −0.236834542 | 0.236834542 | loss | 0.721299119 | 0.484464576 | CDX1 | 37 | 5 |
| cg02524475 | 7.48E−09 | 3.18E−03 | −0.236821228 | 0.236821228 | loss | 0.491501275 | 0.254680047 | HHIP; HHIP-AS1 | 37 | 4 |
| cg11945297 | 3.14E−08 | 1.33E−02 | −0.236804703 | 0.236804703 | loss | 0.821653215 | 0.584848512 | GRHL2 | 37 | 3 |
| cg22753661 | 1.10E−09 | 4.65E−04 | −0.236800795 | 0.236800795 | loss | 0.846742677 | 0.609941882 | ADAMTS7 | 37 | 15 |
| cg17223846 | 7.13E−10 | 3.03E−04 | −0.236793957 | 0.236793957 | loss | 0.632599487 | 0.395805529 | C19orf77; C19orf77 | 37 | 19 |
| cg14243817 | 2.56E−09 | 1.09E−03 | −0.236785494 | 0.236785494 | loss | 0.711255894 | 0.4744704 | | 37 | 9 |
| cg27664085 | 3.40E−08 | 1.44E−02 | −0.236747297 | 0.236747297 | loss | 0.429308794 | 0.192561497 | | 37 | 15 |
| cg15490784 | 1.01E−09 | 4.27E−04 | −0.23671052 | 0.23671052 | loss | 0.443734238 | 0.207023718 | KLF15 | 37 | 3 |
| cg02717339 | 7.13E−10 | 3.03E−04 | −0.236687069 | 0.236687069 | loss | 0.643492234 | 0.406805165 | ANKRD39 | 37 | 2 |
| cg10593400 | 7.13E−10 | 3.03E−04 | −0.236654498 | 0.236654498 | loss | 0.50020194 | 0.263547441 | DDAH1; DDAH1 | 37 | 1 |
| cg06678242 | 5.85E−09 | 2.49E−03 | −0.236651324 | 0.236651324 | loss | 0.725846842 | 0.489195518 | RGMA; RGMA; RGMA; RGMA; | 37 | 15 |
| | | | | | | | | RGMA; RGMA | | |
| cg09240747 | 7.13E−10 | 3.03E−04 | −0.23664331 | 0.23664331 | loss | 0.768484134 | 0.531840824 | | 37 | 17 |
| cg25071744 | 1.01E−09 | 4.27E−04 | −0.23662031 | 0.23662031 | loss | 0.495052834 | 0.258432524 | APOB; APOB | 37 | 2 |
| cg21451816 | 7.13E−10 | 3.03E−04 | −0.236616196 | 0.236616196 | loss | 0.474559391 | 0.237943194 | | 37 | 7 |
| cg13739485 | 7.13E−10 | 3.03E−04 | −0.236603449 | 0.236603449 | loss | 0.828274155 | 0.591670706 | | 37 | 9 |
| cg19025461 | 8.47E−10 | 3.60E−04 | −0.236560327 | 0.236560327 | loss | 0.3518251 | 0.115264773 | FLJ37453 | 37 | 1 |
| cg01067216 | 8.47E−10 | 3.60E−04 | −0.236559367 | 0.236559367 | loss | 0.743083243 | 0.506523876 | | 37 | 14 |
| cg10501602 | 7.13E−10 | 3.03E−04 | −0.2365412 | 0.2365412 | loss | 0.446144394 | 0.209603195 | NOS3 | 37 | 7 |
| cg24439315 | 7.13E−10 | 3.03E−04 | −0.236522566 | 0.236522566 | loss | 0.391646026 | 0.155123461 | LRRC4B | 37 | 19 |
| cg04821993 | 7.13E−10 | 3.03E−04 | −0.236482315 | 0.236482315 | loss | 0.367981698 | 0.131499383 | | 37 | 7 |
| cg18773260 | 2.48E−08 | 1.05E−02 | −0.236420526 | 0.236420526 | loss | 0.564365679 | 0.327945153 | HOXB7 | 37 | 17 |
| cg10143507 | 7.13E−10 | 3.03E−04 | −0.236418002 | 0.236418002 | loss | 0.365903496 | 0.129485494 | | 37 | 2 |
| cg13750214 | 7.84E−10 | 3.33E−04 | −0.236417288 | 0.236417288 | loss | 0.983308729 | 0.746891441 | | 37 | 19 |
| cg20966270 | 9.23E−10 | 3.92E−04 | −0.236407463 | 0.236407463 | loss | 0.742304345 | 0.505896882 | KCNC4; KCNC4; KCNC4 | 37 | 1 |
| cg15692360 | 7.13E−10 | 3.03E−04 | −0.236372011 | 0.236372011 | loss | 0.548082023 | 0.311710012 | FERMT1; FERMT1 | 37 | 20 |
| cg05227350 | 7.13E−10 | 3.03E−04 | −0.236357962 | 0.236357962 | loss | 0.311730979 | 0.075373017 | ZBTB44 | 37 | 11 |
| cg06255037 | 7.13E−10 | 3.03E−04 | −0.236345951 | 0.236345951 | loss | 0.719073409 | 0.482727459 | CHADL | 37 | 22 |
| cg00616135 | 9.23E−10 | 3.92E−04 | −0.236339283 | 0.236339283 | loss | 0.862073072 | 0.625733788 | LACTB; LACTB | 37 | 15 |
| cg09997546 | 1.19E−09 | 5.07E−04 | −0.236330441 | 0.236330441 | loss | 0.537690917 | 0.301360476 | AKIP1; AKIP1; AKIP1; ST5; AKIP1; | 37 | 11 |
| | | | | | | | | AKIP1; AKIP1 | | |
| cg20017761 | 7.13E−10 | 3.03E−04 | −0.236317964 | 0.236317964 | loss | 0.643615028 | 0.407297065 | LOC284798; LOC284798; LOC284798; | 37 | 20 |
| | | | | | | | | LOC284798 | | |
| cg14836960 | 7.77E−10 | 3.30E−04 | −0.236210421 | 0.236210421 | loss | 0.35626384 | 0.120053418 | DPPA4; DPPA4 | 37 | 3 |
| cg08474334 | 7.77E−10 | 3.30E−04 | −0.236192515 | 0.236192515 | loss | 0.628262557 | 0.392070041 | NTRK1; INSRR | 37 | 1 |
| cg19313402 | 7.13E−10 | 3.03E−04 | −0.236189924 | 0.236189924 | loss | 0.643632877 | 0.407442953 | SLC30A10; RNU5F- | 37 | 1 |
| | | | | | | | | 1; SLC30A10 | | |
| cg26799250 | 7.13E−10 | 3.03E−04 | −0.23613919 | 0.23613919 | loss | 0.790571366 | 0.554432176 | PITPNM3; PITPNM3 | 37 | 17 |
| cg11783602 | 1.01E−09 | 4.27E−04 | −0.236090663 | 0.236090663 | loss | 0.679298751 | 0.443208088 | | 37 | 8 |
| cg01550017 | 3.18E−09 | 1.35E−03 | −0.236089292 | 0.236089292 | loss | 0.734883492 | 0.4987942 | | 37 | 5 |
| cg09970967 | 7.13E−10 | 3.03E−04 | −0.23607751 | 0.23607751 | loss | 0.709588434 | 0.473510924 | PSEN1; PSEN1 | 37 | 14 |
| cg19572055 | 3.02E−09 | 1.28E−03 | −0.23603385 | 0.23603385 | loss | 0.703307615 | 0.467273765 | | 37 | 8 |
| cg26214645 | 7.13E−10 | 3.03E−04 | −0.236014538 | 0.236014538 | loss | 0.757101174 | 0.521086635 | SECTM1; SECTM1 | 37 | 17 |
| cg20129827 | 2.16E−09 | 9.17E−04 | −0.235981476 | 0.235981476 | loss | 0.674310506 | 0.438329029 | KIAA1210; KIAA1210 | 37 | X |
| cg25472530 | 2.03E−08 | 8.64E−03 | −0.235981391 | 0.235981391 | loss | 0.606526723 | 0.370545331 | PRDM9 | 37 | 5 |
| cg01030476 | 9.23E−10 | 3.92E−04 | −0.235951993 | 0.235951993 | loss | 0.69850854 | 0.462556547 | LGALS8; LGALS8; LGALS8; LGALS8; | 37 | 1 |
| | | | | | | | | LGALS8-AS1 | | |
| cg13260758 | 1.99E−09 | 8.43E−04 | −0.235922914 | 0.235922914 | loss | 0.726140743 | 0.490217829 | | 37 | 22 |
| cg08354681 | 7.77E−10 | 3.30E−04 | −0.235913213 | 0.235913213 | loss | 0.789961677 | 0.554048465 | CDYL | 37 | 6 |
| cg26904169 | 1.68E−09 | 7.12E−04 | −0.235892888 | 0.235892888 | loss | 0.542592464 | 0.306699576 | STEAP2; STEAP2; STEAP2; STEAP2; | 37 | 7 |
| | | | | | | | | STEAP2 | | |
| cg02051924 | 7.13E−10 | 3.03E−04 | −0.235830009 | 0.235830009 | loss | 0.773308462 | 0.537478453 | LITAF; LITAF; LITAF; LITAF; LITAF | 37 | 16 |
| cg21710255 | 1.12E−08 | 4.76E−03 | −0.235812752 | 0.235812752 | loss | 0.69417697 | 0.458364218 | CRISP2; CRISP2; CRISP2; CRISP2; | 37 | 6 |
| | | | | | | | | CRISP2; CRISP2 | | |
| cg02734505 | 2.16E−09 | 9.17E−04 | −0.235787244 | 0.235787244 | loss | 0.419565857 | 0.183778612 | ZNF385A; ZNF385A; ZNF385A | 37 | 12 |
| cg22109464 | 3.02E−09 | 1.28E−03 | −0.235749538 | 0.235749538 | loss | 0.590820532 | 0.355070994 | C21orf54 | 37 | 21 |
| cg12352302 | 7.13E−10 | 3.03E−04 | −0.235731329 | 0.235731329 | loss | 0.445870523 | 0.210139194 | | 37 | 5 |
| cg01145903 | 9.23E−10 | 3.92E−04 | −0.235718399 | 0.235718399 | loss | 0.886717317 | 0.650998918 | | 37 | 5 |
| cg06837568 | 3.67E−08 | 1.56E−02 | −0.235701483 | 0.235701483 | loss | 0.553914436 | 0.318212953 | WSCD2 | 37 | 12 |
| cg00652772 | 7.13E−10 | 3.03E−04 | −0.235692215 | 0.235692215 | loss | 0.363751489 | 0.128059274 | | 37 | 2 |
| cg08530064 | 7.13E−10 | 3.03E−04 | −0.235668241 | 0.235668241 | loss | 0.445002458 | 0.209334218 | | 37 | 7 |
| cg13353050 | 2.35E−09 | 9.98E−04 | −0.235628349 | 0.235628349 | loss | 0.917760449 | 0.6821321 | | 37 | 3 |
| cg04467334 | 1.99E−09 | 8.43E−04 | −0.235596444 | 0.235596444 | loss | 0.723169832 | 0.487573388 | CCKBR | 37 | 11 |
| cg11590282 | 7.13E−10 | 3.03E−04 | −0.235594395 | 0.235594395 | loss | 0.731996389 | 0.496401994 | | 37 | 11 |
| cg06357843 | 6.89E−09 | 2.93E−03 | −0.235548384 | 0.235548384 | loss | 0.941453502 | 0.705905118 | PANX2; PANX2; PANX2 | 37 | 22 |
| cg15180789 | 7.13E−10 | 3.03E−04 | −0.235513901 | 0.235513901 | loss | 0.455845072 | 0.220331171 | SCHIP1; SCHIP1; SCHIP1; IQCJ- | 37 | 3 |
| | | | | | | | | SCHIP1; IQCJ- | | |
| | | | | | | | | SCHIP1; SCHIP1 | | |
| cg15032861 | 7.13E−10 | 3.03E−04 | −0.235480258 | 0.235480258 | loss | 0.856708511 | 0.621228253 | | 37 | 2 |
| cg21117508 | 3.40E−08 | 1.44E−02 | −0.235432612 | 0.235432612 | loss | 0.805445989 | 0.570013376 | | 37 | 1 |
| cg15895197 | 7.13E−10 | 3.03E−04 | −0.235409582 | 0.235409582 | loss | 0.518530211 | 0.283120629 | EMILIN1 | 37 | 2 |
| cg12515485 | 7.13E−10 | 3.03E−04 | −0.235407895 | 0.235407895 | loss | 0.664973419 | 0.429565524 | LRCOL1 | 37 | 12 |
| cg04289036 | 7.13E−10 | 3.03E−04 | −0.235374414 | 0.235374414 | loss | 0.778494955 | 0.543120541 | C1orf210; C1orf210; C1orf210; | 37 | 1 |
| | | | | | | | | C1orf210 | | |
| cg16530382 | 8.47E−10 | 3.60E−04 | −0.235372894 | 0.235372894 | loss | 0.344464602 | 0.109091708 | FBXO47; FBXO47 | 37 | 17 |
| cg08715146 | 7.13E−10 | 3.03E−04 | −0.235359163 | 0.235359163 | loss | 0.715049651 | 0.479690488 | ATXN10; ATXN10 | 37 | 22 |
| cg14793137 | 1.30E−09 | 5.52E−04 | −0.235353047 | 0.235353047 | loss | 0.729547894 | 0.494194847 | TPO; TPO; TPO; TPO; TPO; TPO | 37 | 2 |
| cg04224786 | 4.57E−09 | 1.94E−03 | −0.23534148 | 0.23534148 | loss | 0.595631068 | 0.360289588 | | 37 | 8 |
| cg05382497 | 1.83E−09 | 7.75E−04 | −0.235317572 | 0.235317572 | loss | 0.752232202 | 0.516914629 | MRGPRE; MRGPRE | 37 | 11 |
| cg04252915 | 7.13E−10 | 3.03E−04 | −0.235313271 | 0.235313271 | loss | 0.868712236 | 0.633398965 | | 37 | 2 |
| cg16450309 | 7.13E−10 | 3.03E−04 | −0.235313054 | 0.235313054 | loss | 0.872145913 | 0.636832859 | EIF5A2 | 37 | 3 |
| cg25918821 | 1.32E−09 | 5.62E−04 | −0.235308662 | 0.235308662 | loss | 0.622737415 | 0.387428753 | | 37 | 17 |
| cg00538923 | 7.84E−10 | 3.33E−04 | −0.235298544 | 0.235298544 | loss | 0.885126767 | 0.649828224 | PRCD; PRCD | 37 | 17 |
| cg05188837 | 7.13E−10 | 3.03E−04 | −0.235261115 | 0.235261115 | loss | 0.530611857 | 0.295350741 | | 37 | 16 |
| cg18771955 | 7.13E−10 | 3.03E−04 | −0.235255166 | 0.235255166 | loss | 0.778938777 | 0.543683612 | | 37 | 2 |
| cg11540016 | 7.48E−09 | 3.18E−03 | −0.235249218 | 0.235249218 | loss | 0.86197713 | 0.626727912 | | 37 | 10 |
| cg05149097 | 7.13E−10 | 3.03E−04 | −0.235208767 | 0.235208767 | loss | 0.457738296 | 0.222529529 | | 37 | 11 |
| cg08206906 | 1.54E−09 | 6.54E−04 | −0.235204381 | 0.235204381 | loss | 0.806495245 | 0.571290865 | | 37 | 19 |
| cg26234644 | 1.99E−09 | 8.43E−04 | −0.235142138 | 0.235142138 | loss | 0.834897291 | 0.599755153 | TMEM220; TMEM220- | 37 | 17 |
| | | | | | | | | AS1; TMEM220- | | |
| | | | | | | | | AS1; TMEM220- | | |
| | | | | | | | | AS1; TMEM220-AS1 | | |
| cg25985959 | 7.77E−10 | 3.30E−04 | −0.235099858 | 0.235099858 | loss | 0.845290928 | 0.610191071 | | 37 | 6 |
| cg21459583 | 7.13E−10 | 3.03E−04 | −0.235097157 | 0.235097157 | loss | 0.642477451 | 0.407380294 | | 37 | 1 |
| cg00339913 | 1.99E−09 | 8.43E−04 | −0.235083413 | 0.235083413 | loss | 0.687986013 | 0.4529026 | PHYHIP; PHYHIP | 37 | 8 |
| cg07267550 | 7.13E−10 | 3.03E−04 | −0.235075321 | 0.235075321 | loss | 0.976426268 | 0.741350947 | PRCD; PRCD | 37 | 17 |
| cg01689404 | 1.01E−09 | 4.27E−04 | −0.235075164 | 0.235075164 | loss | 0.45953687 | 0.224461706 | | 37 | 17 |
| cg09581049 | 7.13E−10 | 3.03E−04 | −0.235072364 | 0.235072364 | loss | 0.394342111 | 0.159269747 | | 37 | 17 |
| cg22670819 | 7.13E−10 | 3.03E−04 | −0.235057927 | 0.235057927 | loss | 0.538189974 | 0.303132047 | CFB | 37 | 6 |
| cg08433293 | 7.13E−10 | 3.03E−04 | −0.235039768 | 0.235039768 | loss | 0.483775321 | 0.248735553 | FANCI; FANCI | 37 | 15 |
| cg24334149 | 2.56E−09 | 1.09E−03 | −0.235028294 | 0.235028294 | loss | 0.691907158 | 0.456878865 | ATP6V1B1 | 37 | 2 |
| cg15951661 | 7.77E−10 | 3.30E−04 | −0.234996868 | 0.234996868 | loss | 0.688297804 | 0.453300935 | MAP3K9; MAP3K9 | 37 | 14 |
| cg10502231 | 7.77E−10 | 3.30E−04 | −0.23492732 | 0.23492732 | loss | 0.346243787 | 0.111316466 | | 37 | 1 |
| cg05879418 | 4.57E−09 | 1.94E−03 | −0.234920022 | 0.234920022 | loss | 0.422920128 | 0.188000106 | GPX8; CDC20B; CDC20B; CDC20B | 37 | 5 |
| cg14121987 | 5.85E−09 | 2.49E−03 | −0.234914267 | 0.234914267 | loss | 0.833722379 | 0.598808112 | | 37 | 5 |
| cg07147285 | 7.13E−10 | 3.03E−04 | −0.2348684 | 0.2348684 | loss | 0.785210306 | 0.550341906 | CCDC113; CCDC113 | 37 | 16 |
| cg10505973 | 1.30E−09 | 5.52E−04 | −0.234850624 | 0.234850624 | loss | 0.70654943 | 0.471698806 | | 37 | 14 |
| cg04865726 | 1.54E−09 | 6.54E−04 | −0.234850254 | 0.234850254 | loss | 0.343797577 | 0.108947324 | | 37 | 1 |
| cg22697853 | 1.99E−09 | 8.43E−04 | −0.234839995 | 0.234839995 | loss | 0.51314403 | 0.278304035 | N8PF8; NBPF8; NBPF8 | 37 | 1 |
| cg14051236 | 7.13E−10 | 3.03E−04 | −0.23480544 | 0.23480544 | loss | 0.446550528 | 0.211745088 | CCDC85C | 37 | 14 |
| cg20699305 | 7.13E−10 | 3.03E−04 | −0.234781663 | 0.234781663 | loss | 0.792635351 | 0.557853688 | ACTL8 | 37 | 1 |
| cg25714115 | 7.13E−10 | 3.03E−04 | −0.23477483 | 0.23477483 | loss | 0.362204432 | 0.127429602 | GTF2IRD1; GTF2IRD1; GTF2IRD1 | 37 | 7 |
| cg12904135 | 1.32E−08 | 5.59E−03 | −0.234757765 | 0.234757765 | loss | 0.8071261 | 0.572368335 | | 37 | 2 |
| cg20623503 | 7.13E−10 | 3.03E−04 | −0.234657013 | 0.234657013 | loss | 0.900750577 | 0.666093565 | CARD14; CARD14; CARD14 | 37 | 17 |
| cg02582520 | 4.21E−09 | 1.79E−03 | −0.234648572 | 0.234648572 | loss | 0.750129619 | 0.515481047 | GRIN3B | 37 | 19 |
| cg23659134 | 1.19E−09 | 5.07E−04 | −0.234636509 | 0.234636509 | loss | 0.763343662 | 0.528707153 | GSTO2; GSTO2; GSTO2; GSTO2 | 37 | 10 |
| cg15959252 | 1.68E−09 | 7.12E−04 | −0.234574729 | 0.234574729 | loss | 0.50297847 | 0.268403741 | C12orf68 | 37 | 12 |
| cg01644795 | 1.42E−09 | 6.01E−04 | −0.234572726 | 0.234572726 | loss | 0.493469791 | 0.258897065 | GATA4; C8orf49 | 37 | 8 |
| cg03716984 | 2.35E−09 | 9.98E−04 | −0.234563917 | 0.234563917 | loss | 0.811949458 | 0.577385541 | | 37 | 10 |
| cg11604426 | 7.13E−10 | 3.03E−04 | −0.234497641 | 0.234497641 | loss | 0.413262364 | 0.178764724 | RHBDL3 | 37 | 17 |
| cg01905210 | 7.13E−10 | 3.03E−04 | −0.234462004 | 0.234462004 | loss | 0.539855998 | 0.305393994 | ESYT3 | 37 | 3 |
| cg14844953 | 1.01E−09 | 4.27E−04 | −0.234461954 | 0.234461954 | loss | 0.824321825 | 0.589859871 | | 37 | 1 |
| cg00012362 | 2.35E−09 | 9.98E−04 | −0.234434436 | 0.234434436 | loss | 0.684804789 | 0.450370353 | S100A13; S100A13; S100A13; | 37 | 1 |
| | | | | | | | | S100A13; S100A13; S100A13; | | |
| | | | | | | | | S100A1 | | |
| cg10907148 | 4.97E−09 | 2.11E−03 | −0.234410865 | 0.234410865 | loss | 0.80576843 | 0.571357565 | HID1 | 37 | 17 |
| cg02303258 | 7.13E−10 | 3.03E−04 | −0.234401238 | 0.234401238 | loss | 0.842138021 | 0.607736782 | | 37 | 5 |
| cg13886554 | 7.13E−10 | 3.03E−04 | −0.234401176 | 0.234401176 | loss | 0.353977436 | 0.119576259 | FBLN7; FBLN7 | 37 | 2 |
| cg00962755 | 7.13E−10 | 3.03E−04 | −0.23438919 | 0.23438919 | loss | 0.403446066 | 0.169056876 | PHLDA3; PHLDA3 | 37 | 1 |
| cg25740457 | 1.68E−09 | 7.12E−04 | −0.234380905 | 0.234380905 | loss | 0.523992075 | 0.289611171 | CCKBR | 37 | 11 |
| cg03044367 | 7.13E−10 | 3.03E−04 | −0.234379725 | 0.234379725 | loss | 0.699620519 | 0.465240794 | | 37 | 1 |
| cg17641252 | 1.43E−08 | 6.06E−03 | −0.234369022 | 0.234369022 | loss | 0.574850898 | 0.340481876 | PLEK2 | 37 | 14 |
| cg13491480 | 7.13E−10 | 3.03E−04 | −0.234360848 | 0.234360848 | loss | 0.646540066 | 0.412179218 | LOC100289019 | 37 | 9 |
| cg24854181 | 3.39E−09 | 1.44E−03 | −0.234336288 | 0.234336288 | loss | 0.890914517 | 0.656578229 | | 37 | 5 |
| cg20356482 | 7.13E−10 | 3.03E−04 | −0.234316418 | 0.234316418 | loss | 0.893205642 | 0.658889224 | FBP2; FBP2 | 37 | 9 |
| cg19317226 | 2.91E−08 | 1.23E−02 | −0.234287592 | 0.234287592 | loss | 0.728625992 | 0.4943384 | | 37 | 10 |
| cg05124243 | 9.23E−10 | 3.92E−04 | −0.234267976 | 0.234267976 | loss | 0.918483111 | 0.684215135 | | 37 | 10 |
| cg26929272 | 7.13E−10 | 3.03E−04 | −0.234264307 | 0.234264307 | loss | 0.900541266 | 0.666276959 | | 37 | 10 |
| cg16627090 | 2.35E−09 | 9.98E−04 | −0.23425742 | 0.23425742 | loss | 0.422534396 | 0.188276976 | PGAM2; PGAM2 | 37 | 7 |
| cg21936179 | 2.35E−09 | 9.98E−04 | −0.234242392 | 0.234242392 | loss | 0.520414762 | 0.286172371 | EIF2D; EIF2D | 37 | 1 |
| cg06789686 | 9.23E−10 | 3.92E−04 | −0.234240118 | 0.234240118 | loss | 0.822273883 | 0.588033765 | | 37 | 17 |
| cg27239981 | 1.10E−09 | 4.65E−04 | −0.23422862 | 0.23422862 | loss | 0.576431849 | 0.342203229 | | 37 | 6 |
| cg22953960 | 7.77E−10 | 3.30E−04 | −0.234217984 | 0.234217984 | loss | 0.502214783 | 0.267996799 | | 37 | 10 |
| cg07472579 | 8.47E−10 | 3.60E−04 | −0.2342005 | 0.2342005 | loss | 0.7935202 | 0.5593197 | | 37 | 6 |
| cg19925872 | 7.13E−10 | 3.03E−04 | −0.23419881 | 0.23419881 | loss | 0.860072157 | 0.625873347 | ZNF710 | 37 | 15 |
| cg04582672 | 1.99E−09 | 8.43E−04 | −0.234183026 | 0.234183026 | loss | 0.778744885 | 0.544561859 | | 37 | 14 |
| cg03856044 | 4.64E−08 | 1.97E−02 | −0.234175334 | 0.234175334 | loss | 0.850198775 | 0.616023441 | APCS | 37 | 1 |
| cg26878893 | 7.13E−10 | 3.03E−04 | −0.234172063 | 0.234172063 | loss | 0.423001792 | 0.188829729 | ALX3 | 37 | 1 |
| cg02852670 | 1.10E−09 | 4.65E−04 | −0.234141094 | 0.234141094 | loss | 0.9562343 | 0.722093206 | KLHL33 | 37 | 14 |
| cg14087574 | 7.13E−10 | 3.03E−04 | −0.234138569 | 0.234138569 | loss | 0.797036545 | 0.562897976 | | 37 | 1 |
| cg05657709 | 3.02E−09 | 1.28E−03 | −0.234134206 | 0.234134206 | loss | 0.578022606 | 0.3438884 | ILDR1; ILDR1; ILDR1 | 37 | 3 |
| cg16044810 | 7.13E−10 | 3.03E−04 | −0.234126517 | 0.234126517 | loss | 0.813344817 | 0.5792183 | CRYGN | 37 | 7 |
| cg13952899 | 1.99E−09 | 8.43E−04 | −0.234109668 | 0.234109668 | loss | 0.789396715 | 0.555287047 | C1orf95 | 37 | 1 |
| cg06483432 | 7.13E−10 | 3.03E−04 | −0.234105438 | 0.234105438 | loss | 0.522125474 | 0.288020035 | KIT; KIT | 37 | 4 |
| cg00404187 | 7.13E−10 | 3.03E−04 | −0.234103139 | 0.234103139 | loss | 0.572270815 | 0.338167676 | | 37 | 7 |
| cg15399533 | 7.13E−10 | 3.03E−04 | −0.234097048 | 0.234097045 | loss | 0.581423583 | 0.347326535 | NR2E1; NR2E1 | 37 | 6 |
| cg07278425 | 3.88E−09 | 1.65E−03 | −0.234096729 | 0.234096729 | loss | 0.616959117 | 0.382862388 | HOTAIRM1; KOTAIRM1 | 37 | 7 |
| cg16277071 | 2.35E−09 | 9.98E−04 | −0.234095695 | 0.234095696 | loss | 0.526598202 | 0.292502506 | | 37 | 17 |
| cg18888461 | 1.99E−09 | 8.43E−04 | −0.23408929 | 0.23408929 | loss | 0.405655496 | 0.171566206 | LDHAL6A; LDHAL6A | 37 | 11 |
| cg21144120 | 7.13E−10 | 3.03E−04 | −0.234009118 | 0.234009118 | loss | 0.353662134 | 0.119653016 | | 37 | 6 |
| cg20378690 | 4.57E−09 | 1.94E−03 | −0.23399083 | 0.23399083 | loss | 0.545896177 | 0.311905347 | PEBP4 | 37 | 8 |
| cg23973272 | 3.40E−08 | 1.44E−02 | −0.233958269 | 0.233958269 | loss | 0.774365998 | 0.540407729 | PXDNL | 37 | 8 |
| cg12905571 | 5.39E−09 | 2.29E−03 | −0.233925727 | 0.233925727 | loss | 0.415757309 | 0.181831582 | | 37 | 1 |
| cg10910512 | 1.42E−09 | 6.01E−04 | −0.233907994 | 0.233907994 | loss | 0.736310853 | 0.502402859 | LAMA4; LAMA4; LAMA4; LAMA4; | 37 | 6 |
| | | | | | | | | LAMA4; LAMA4; LAMA4; | | |
| | | | | | | | | LAMA4; LAMA4; LAMA4 | | |
| cg10186366 | 7.13E−10 | 3.03E−04 | −0.233885105 | 0.233885105 | loss | 0.328431283 | 0.094546178 | | 37 | 17 |
| cg14644418 | 1.03E−08 | 4.39E−03 | −0.233883886 | 0.233883886 | loss | 0.697999615 | 0.464115729 | FOXI2 | 37 | 10 |
| cg05266836 | 2.91E−08 | 1.23E−02 | −0.23388253 | 0.23388253 | loss | 0.659156277 | 0.425273747 | | 37 | 17 |
| cg08363684 | 1.03E−08 | 4.39E−03 | −0.233877161 | 0.233877161 | loss | 0.967813243 | 0.733936082 | | 37 | 6 |
| cg02008416 | 3.57E−09 | 1.52E−03 | −0.233876658 | 0.233876658 | loss | 0.78368357 | 0.549806912 | TMEM61 | 37 | 1 |
| cg23948204 | 7.13E−10 | 3.03E−04 | −0.233870744 | 0.233870744 | loss | 0.580432485 | 0.346561741 | | 37 | 5 |
| cg11183181 | 2.35E−09 | 9.98E−04 | −0.233863344 | 0.233863344 | loss | 0.853003268 | 0.619139924 | | 37 | 5 |
| cg01692252 | 7.77E−10 | 3.30E−04 | −0.233859018 | 0.233859018 | loss | 0.773096594 | 0.539237576 | GRB7 | 37 | 17 |
| cg25976170 | 1.83E−09 | 7.75E−04 | −0.233847273 | 0.233847273 | loss | 0.900607891 | 0.666760618 | GAS2L1; GAS2L1; GAS2L1 | 37 | 22 |
| cg18432895 | 1.19E−09 | 5.07E−04 | −0.233835855 | 0.233835855 | loss | 0.631086438 | 0.397250582 | DUSP4; DUSP4 | 37 | 8 |
| cg05994850 | 2.78E−09 | 1.18E−03 | −0.233828663 | 0.233828663 | loss | 0.595303034 | 0.361474371 | | 37 | 6 |
| cg00110832 | 7.13E−10 | 3.03E−04 | −0.233825431 | 0.233825431 | loss | 0.882945002 | 0.649119571 | PPT2; PPT2; PRRT1; PPT2; LOC100507547; | 37 | 6 |
| | | | | | | | | LOC100507547; | | |
| | | | | | | | | LOC100507547; LOC100507547; | | |
| | | | | | | | | PPT2-EGFL8 | | |
| cg08638582 | 2.35E−09 | 9.98E−04 | −0.233828312 | 0.233818312 | loss | 0.754796994 | 0.520978682 | DIRAS1 | 37 | 19 |
| cg22285914 | 2.48E−08 | 1.05E−02 | −0.233794236 | 0.233794236 | loss | 0.603427442 | 0.369633206 | SP7; SP7; SP7 | 37 | 12 |
| cg04857403 | 7.13E−10 | 3.03E−04 | −0.233762817 | 0.233762817 | loss | 0.443605675 | 0.209842859 | | 37 | 1 |
| cg26410635 | 6.89E−09 | 2.93E−03 | −0.233742931 | 0.233742931 | loss | 0.725576355 | 0.491833424 | AMDHD1 | 37 | 12 |
| cg04387820 | 3.57E−09 | 1.52E−03 | −0.233728631 | 0.233728631 | loss | 0.620687913 | 0.386959282 | | 37 | 10 |
| cg15458577 | 7.13E−10 | 3.03E−04 | −0.233723103 | 0.233723103 | loss | 0.643481779 | 0.409758676 | | 37 | 1 |
| cg17480077 | 7.13E−10 | 3.03E−04 | −0.233715642 | 0.233715642 | loss | 0.364898742 | 0.1311831 | | 37 | 1 |
| cg08032971 | 1.19E−09 | 5.07E−04 | −0.233707297 | 0.233707297 | loss | 0.853910726 | 0.620203429 | DMBX1; DMBX1 | 37 | 1 |
| cg14195249 | 7.13E−10 | 3.03E−04 | −0.23370314 | 0.23370314 | loss | 0.735202675 | 0.501499535 | | 37 | ID |
| cg24977886 | 7.13E−10 | 3.03E−04 | −0.23364877 | 0.23364877 | loss | 0.654826453 | 0.421177682 | FAM109B | 37 | 22 |
| cg02624051 | 1.30E−09 | 5.52E−04 | −0.233645051 | 0.233645051 | loss | 0.862620604 | 0.628975553 | PLA2G4D | 37 | 15 |
| cg13993183 | 1.10E−09 | 4.65E−04 | −0.233615163 | 0.233615163 | loss | 0.875791998 | 0.642176835 | | 37 | 9 |
| cg27487796 | 1.54E−09 | 6.54E−04 | −0.233613509 | 0.233613509 | loss | 0.754440779 | 0.520827271 | | 37 | 7 |
| cg11194613 | 8.11E−09 | 3.44E−03 | −0.233565155 | 0.233565155 | loss | 0.725724991 | 0.492159835 | CTTN; CTTN; CTTN | 37 | 11 |
| cg27649348 | 7.77E−10 | 3.30E−04 | −0.233547346 | 0.233547346 | loss | 0.460318134 | 0.226770788 | | 37 | 5 |
| cg07889505 | 1.99E−09 | 8.43E−04 | −0.233542563 | 0.233542563 | loss | 0.401959581 | 0.168417018 | | 37 | 15 |
| cg00491927 | 7.13E−10 | 3.03E−04 | −0.233540654 | 0.233540654 | loss | 0.345683728 | 0.112143075 | | 37 | 3 |
| cg19655177 | 7.13E−10 | 3.03E−04 | −0.233534743 | 0.233534743 | loss | 0.756371843 | 0.5228371 | | 37 | 7 |
| cg19021383 | 7.13E−10 | 3.03E−04 | −0.233525728 | 0.233525728 | loss | 0.459785725 | 0.226259996 | MAPK8IP1 | 37 | 11 |
| cg02596940 | 7.13E−10 | 3.03E−04 | −0.233494505 | 0.233494505 | loss | 0.441589181 | 0.208094676 | | 37 | 4 |
| cg18461362 | 8.47E−10 | 3.60E−04 | −0.233456235 | 0.233456235 | loss | 0.8375265 | 0.604070265 | | 37 | 8 |
| cg02072875 | 7.13E−10 | 3.03E−04 | −0.233433024 | 0.233433024 | loss | 0.848812847 | 0.615379824 | IQCE; IQCE | 37 | 7 |
| cg21171339 | 7.48E−09 | 3.18E−03 | −0.233432349 | 0.233432349 | loss | 0.559339013 | 0.325906665 | VPS13A; VPS13A; VPS13A; | 37 | 9 |
| | | | | | | | | VPS13A | | |
| cg15429214 | 1.19E−09 | 5.07E−04 | −0.233432017 | 0.233432017 | loss | 0.795414817 | 0.5619828 | | 37 | 22 |
| cg19691659 | 7.13E−10 | 3.03E−04 | −0.233430258 | 0.233430258 | loss | 0.723174128 | 0.489743871 | NRXN2; NRXN2 | 37 | 11 |
| cg05181941 | 2.16E−09 | 9.17E−04 | −0.23342766 | 0.23342766 | loss | 0.85008406 | 0.6166564 | | 37 | 12 |
| cg09728985 | 7.13E−10 | 3.03E−04 | −0.233426065 | 0.233426065 | loss | 0.81284573 | 0.579419665 | | 37 | 16 |
| cg10654546 | 8.56E−10 | 3.63E−04 | −0.233390536 | 0.233390536 | loss | 0.326887129 | 0.093496592 | NABP2 | 37 | 12 |
| cg23464360 | 7.13E−10 | 3.03E−04 | −0.23339002 | 0.23339002 | loss | 0.543436049 | 0.310046029 | MAFA | 37 | 8 |
| cg13207534 | 2.56E−09 | 1.09E−03 | −0.233383655 | 0.233383655 | loss | 0.848617549 | 0.615233894 | PPP1R3G | 37 | 6 |
| cg09580336 | 7.13E−10 | 3.03E−04 | −0.233381375 | 0.233381375 | loss | 0.370309045 | 0.136927671 | ATP1A1; ATP1A1 | 37 | 1 |
| cg24001246 | 7.13E−10 | 3.03E−04 | −0.233313921 | 0.233313921 | loss | 0.620019557 | 0.386705635 | RAP1GAP; RAP1GAP; RAP1GAP | 37 | 1 |
| cg24264254 | 7.13E−10 | 3.03E−04 | −0.233301747 | 0.233301747 | loss | 0.842949242 | 0.609647494 | | 37 | 10 |
| cg27158481 | 7.13E−10 | 3.03E−04 | −0.233284187 | 0.233284187 | loss | 0.60637467 | 0.373090482 | LOC284798; LOC284798; LOC284798; | 37 | 20 |
| | | | | | | | | LOC284798 | | |
| cg24308847 | 7.13E−10 | 3.03E−04 | −0.23326874 | 0.23326874 | loss | 0.449561228 | 0.216292488 | | 37 | 20 |
| cg18363035 | 1.99E−09 | 8.43E−04 | −0.233252495 | 0.233252495 | loss | 0.457496272 | 0.224243776 | GJB6; GJB6; GJB6; GJB6; GJB6 | 37 | 13 |
| cg16903060 | 5.01E−08 | 2.13E−02 | −0.233246757 | 0.233246757 | loss | 0.829945792 | 0.596699035 | | 37 | 1 |
| cg04440361 | 1.54E−09 | 6.54E−04 | −0.233225099 | 0.233225099 | loss | 0.715425387 | 0.482200288 | | 37 | 17 |
| cg02964065 | 4.97E−09 | 2.11E−03 | −0.233200655 | 0.233200655 | loss | 0.730735902 | 0.497535247 | HLA-DQB2 | 37 | 6 |
| cg19091930 | 7.13E−10 | 3.03E−04 | −0.233196398 | 0.233196398 | loss | 0.909585745 | 0.676389347 | LRRC14B | 37 | 5 |
| cg13598010 | 7.13E−10 | 3.03E−04 | −0.233141667 | 0.233141667 | loss | 0.277015602 | 0.043873935 | | 37 | 7 |
| cg10432422 | 8.47E−10 | 3.60E−04 | −0.233134658 | 0.233134658 | loss | 0.467853075 | 0.234718418 | | 37 | 1 |
| cg07511721 | 1.01E−09 | 4.27E−04 | −0.233132989 | 0.233132989 | loss | 0.375275079 | 0.14214209 | SLC29A4; SLC29A4 | 37 | 7 |
| cg22956649 | 2.48E−08 | 1.05E−02 | −0.233121988 | 0.233121988 | loss | 0.805262806 | 0.572140818 | HOXD-AS1 | 37 | 2 |
| cg01109337 | 2.35E−09 | 9.98E−04 | −0.233119293 | 0.233119293 | loss | 0.420963987 | 0.187844694 | TEX14; TEX14; TEX14 | 37 | 17 |
| cg04690110 | 7.13E−10 | 3.03E−04 | −0.23311402 | 0.23311402 | loss | 0.409984449 | 0.176870429 | | 37 | 6 |
| cg16392860 | 7.13E−10 | 3.03E−04 | −0.23309474 | 0.23309474 | loss | 0.687301328 | 0.454206588 | GRM4; GRM4; GRM4; GRM4 | 37 | 6 |
| cg07545858 | 7.13E−10 | 3.03E−04 | −0.233060781 | 0.233060781 | loss | 0.862014334 | 0.628953553 | | 37 | 16 |
| cg25969122 | 4.64E−08 | 1.97E−02 | 0.233029219 | 0.233029219 | gain | 0.172704964 | 0.405734182 | | 37 | 11 |
| cg27222736 | 7.13E−10 | 3.03E−04 | −0.233025464 | 0.233025464 | loss | 0.413774058 | 0.180748594 | | 37 | 6 |
| cg18083764 | 1.19E−09 | 5.07E−04 | −0.233020268 | 0.233020268 | loss | 0.859466526 | 0.626446259 | | 37 | 5 |
| cg21745164 | 8.47E−10 | 3.60E−04 | −0.233003607 | 0.233003607 | loss | 0.639220372 | 0.406216765 | CHP2 | 37 | 16 |
| cg09562655 | 3.02E−09 | 1.28E−03 | −0.232974059 | 0.232974059 | loss | 0.953493 | 0.720518941 | | 37 | 3 |
| cg07547765 | 2.30E−08 | 9.75E−03 | −0.232965631 | 0.232965631 | loss | 0.577854602 | 0.344888971 | HOXB7 | 37 | 17 |
| cg19846667 | 1.54E−09 | 6.54E−04 | −0.232943151 | 0.232943151 | loss | 0.352113809 | 0.119170659 | | 37 | 14 |
| cg13503773 | 9.22E−08 | 3.91E−02 | −0.232918104 | 0.232918104 | loss | 0.944215645 | 0.711297541 | C9orf47; C9orf47; S1PR3 | 37 | 9 |
| cg18303437 | 1.07E−07 | 4.55E−02 | −0.232904567 | 0.232904567 | loss | 0.787853349 | 0.554948782 | | 37 | 3 |
| cg08084655 | 8.47E−10 | 3.60E−04 | −0.232878183 | 0.232878183 | loss | 0.501863594 | 0.268985412 | BTBD17 | 37 | 17 |
| cg01360281 | 7.77E−10 | 3.30E−04 | −0.232868105 | 0.232868105 | loss | 0.882661575 | 0.649793471 | | 37 | 5 |
| cg05194362 | 7.13E−10 | 3.03E−04 | −0.232865349 | 0.232865349 | loss | 0.81055976 | 0.577694412 | PAX8; PAX8; PAX8; PAX8; PAX8; | 37 | 2 |
| | | | | | | | | PAX8; PAX8; PAX8 | | |
| cg00965023 | 1.01E−09 | 4.27E−04 | 0.232840352 | 0.232840352 | gain | 0.239083525 | 0.471923876 | ZNF833P | 37 | 19 |
| cg12353636 | 1.99E−09 | 8.43E−04 | −0.23283697 | 0.23283697 | loss | 0.821439575 | 0.588602606 | CDHR1; CDHR1 | 37 | 10 |
| cg17846127 | 9.23E−10 | 3.92E−04 | −0.232822247 | 0.232822247 | loss | 0.681484153 | 0.448661906 | LINC00925 | 37 | 15 |
| cg21926804 | 7.84E−10 | 3.33E−04 | −0.232807374 | 0.232807374 | loss | 0.784322215 | 0.551514841 | C8orf74 | 37 | 8 |
| cg20883730 | 8.47E−10 | 3.60E−04 | −0.232783658 | 0.232783658 | loss | 0.82212227 | 0.589338612 | NLRP14 | 37 | 11 |
| cg08354406 | 7.13E−10 | 3.03E−04 | −0.232771657 | 0.232771657 | loss | 0.413399934 | 0.180628276 | NRAP; NRAP; NRAP | 37 | 10 |
| cg25851313 | 7.13E−10 | 3.03E−04 | −0.232764793 | 0.232764793 | loss | 0.287022383 | 0.05425759 | FAM131C | 37 | 1 |
| cg21215218 | 1.19E−09 | 5.07E−04 | −0.232747234 | 0.232747234 | loss | 0.589118145 | 0.356370912 | PAX7; PAX7; PAX7 | 37 | 1 |
| cg15420683 | 7.13E−10 | 3.03E−04 | −0.232746904 | 0.232746904 | loss | 0.965454787 | 0.732707882 | TMEM151B | 37 | 6 |
| cg13478289 | 1.10E−09 | 4.65E−04 | −0.232717374 | 0.232717374 | loss | 0.739717768 | 0.507000394 | COL11A2; COL11A2; COL11A2 | 37 | 6 |
| cg04452432 | 7.13E−10 | 3.03E−04 | −0.232714887 | 0.232714887 | loss | 0.850505481 | 0.617790594 | CX3CL1 | 37 | 16 |
| cg04939302 | 1.30E−09 | 5.52E−04 | −0.232683244 | 0.232683244 | loss | 0.720142274 | 0.487459029 | | 37 | 2 |
| cg26567736 | 4.97E−09 | 2.11E−03 | −0.232648577 | 0.232648577 | loss | 0.64962343 | 0.416974853 | NTM | 37 | 11 |
| cg20185617 | 7.13E−10 | 3.03E−04 | −0.232595343 | 0.232595343 | loss | 0.707592943 | 0.4749976 | | 37 | 1 |
| cg03550129 | 7.13E−10 | 3.03E−04 | −0.232564263 | 0.232564263 | loss | 0.547197081 | 0.314632818 | | 37 | 2 |
| cg22683036 | 7.13E−10 | 3.03E−04 | −0.232563094 | 0.232563094 | loss | 0.362875494 | 0.1303124 | ATG4C; ATG4C | 37 | 1 |
| cg08898775 | 1.10E−09 | 4.65E−04 | −0.232537976 | 0.232537976 | loss | 0.7384341 | 0.505896124 | ADAM10 | 37 | 15 |
| cg13688889 | 3.88E−09 | 1.65E−03 | −0.232537423 | 0.232537423 | loss | 0.794987011 | 0.562449588 | | 37 | 9 |
| cg16555417 | 1.01E−09 | 4.27E−04 | −0.232526094 | 0.232526094 | loss | 0.406202964 | 0.173676871 | | 37 | 1 |
| cg25290227 | 3.02E−09 | 1.28E−03 | −0.232499029 | 0.232499029 | loss | 0.589225853 | 0.356726824 | GPAM; GPAM | 37 | 10 |
| cg17512539 | 5.41E−08 | 2.30E−02 | −0.232483095 | 0.232483095 | loss | 0.738668289 | 0.506185194 | | 37 | 6 |
| cg21345949 | 7.13E−10 | 3.03E−04 | −0.232461609 | 0.232461609 | loss | 0.4885799 | 0.256118291 | FUOM; FUOM; FUOM; FUOM | 37 | 10 |
| cg21436572 | 1.32E−08 | 5.59E−03 | −0.232451673 | 0.232451673 | loss | 0.602693443 | 0.370241771 | SLCO5A1; SLCO5A1; SLCOSA1 | 37 | 8 |
| cg22433862 | 7.13E−10 | 3.03E−04 | −0.232446469 | 0.232446469 | loss | 0.949142098 | 0.716695629 | | 37 | 1 |
| cg08590069 | 7.13E−10 | 3.03E−04 | −0.232437846 | 0.232437846 | loss | 0.594141817 | 0.361703971 | RGS22; RGS22; RGS22 | 37 | 8 |
| cg19034028 | 7.13E−10 | 3.03E−04 | −0.232347943 | 0.232347943 | loss | 0.727307355 | 0.494959412 | CDH3 | 37 | 16 |
| cg23239574 | 7.13E−10 | 3.03E−04 | −0.232313085 | 0.232313085 | loss | 0.469965843 | 0.237652759 | | 37 | 1 |
| cg12134633 | 3.97E−08 | 1.69E−02 | −0.232280547 | 0.232280547 | loss | 0.547433394 | 0.315152847 | SCG5; SCG5 | 37 | 35 |
| cg22678073 | 1.01E−09 | 4.27E−04 | −0.232200937 | 0.232200937 | loss | 0.600349713 | 0.368148776 | ERRFI1 | 37 | 1 |
| cg05316627 | 7.13E−10 | 3.03E−04 | −0.232193081 | 0.232193081 | loss | 0.690303934 | 0.458110853 | | 37 | 6 |
| cg09581060 | 7.13E−10 | 3.03E−04 | −0.232178313 | 0.232178313 | loss | 0.788791725 | 0.556613412 | | 37 | 2 |
| cg20059012 | 5.83E−08 | 2.47E−02 | −0.232154012 | 0.232154012 | loss | 0.381738377 | 0.149584365 | RARG; RARG; RARG; RARG; | 37 | 12 |
| | | | | | | | | RARG | | |
| cg25194612 | 3.39E−09 | 1.44E−03 | −0.232151396 | 0.232151396 | loss | 0.799220979 | 0.567069582 | | 37 | 11 |
| cg13015534 | 1.42E−09 | 6.01E−04 | −0.232130938 | 0.232130938 | loss | 0.487875285 | 0.255744347 | ST6GALNAC1; ST6GALNAC1 | 37 | 17 |
| cg05504055 | 1.19E−09 | 5.07E−04 | −0.232120882 | 0.232120882 | loss | 0.846948206 | 0.614827324 | PRSS42; PRSS42 | 37 | 3 |
| cg23800169 | 2.46E−09 | 1.05E−03 | −0.232055964 | 0.232055964 | loss | 0.758587652 | 0.526531688 | | 37 | 10 |
| cg13430464 | 1.68E−09 | 7.12E−04 | −0.232029739 | 0.232029739 | loss | 0.376768455 | 0.144738716 | CHRDL2; CHRDL2 | 37 | 11 |
| cg02621357 | 9.23E−10 | 3.92E−04 | −0.232020285 | 0.232020285 | loss | 0.564966091 | 0.332945806 | FAM167A | 37 | 8 |
| cg11084470 | 7.13E−10 | 3.03E−04 | −0.232000548 | 0.232000548 | loss | 0.57908303 | 0.347082482 | TCP11; TCP11; TCP11; TCP11; | 37 | 6 |
| | | | | | | | | TCP11; TCP11; TCP11 | | |
| cg18815398 | 1.43E−08 | 6.06E−03 | −0.231966642 | 0.231966642 | loss | 0.702626177 | 0.470659535 | | 37 | 20 |
| cg20693690 | 1.67E−08 | 7.10E−03 | −0.231959462 | 0.231959462 | loss | 0.772022821 | 0.540063359 | TCF7L1 | 37 | 2 |
| cg13754720 | 1.54E−09 | 6.54E−04 | −0.231958789 | 0.231958789 | loss | 0.68601533 | 0.454056541 | PTCH1; PTCH1 | 37 | 9 |
| cg10613381 | 7.77E−10 | 3.30E−04 | −0.231934664 | 0.231934664 | loss | 0.713740764 | 0.4818061 | UPB1; ADORA2A- | 37 | 22 |
| | | | | | | | | AS1; ADORA2A-AS1 | | |
| cg07651720 | 1.19E−09 | 5.07E−04 | −0.231932184 | 0.231932184 | loss | 0.951734619 | 0.719802435 | | 37 | 8 |
| cg00943382 | 7.84E−10 | 3.33E−04 | −0.231930384 | 0.231930384 | loss | 0.645996813 | 0.414066429 | | 37 | 7 |
| cg13972661 | 1.10E−09 | 4.65E−04 | −0.231876034 | 0.231876034 | loss | 0.584277328 | 0.352401294 | HIATL2 | 37 | 9 |
| cg10622551 | 4.97E−09 | 2.11E−03 | −0.231873214 | 0.231873214 | loss | 0.333478143 | 0.101604929 | ZFYVE28; ZFYVE28; ZFYVE28; | 37 | 4 |
| | | | | | | | | ZFYVE28; ZFYVE28; ZFYVE28; | | |
| | | | | | | | | ZFYVE28; ZFYVE28 | | |
| cg04493143 | 1.28E−08 | 5.44E−03 | −0.231835301 | 0.231835301 | loss | 0.909983431 | 0.678148129 | HTR4; HTR4; HTR4; HTR4; HTR4; | 37 | 5 |
| | | | | | | | | HTR4; HTR4 | | |
| cg04986555 | 7.13E−10 | 3.03E−04 | −0.231829255 | 0.231829255 | loss | 0.480048702 | 0.248219447 | TMEM61 | 37 | 1 |
| cg00798317 | 7.13E−10 | 3.03E−04 | −0.231800939 | 0.231800939 | loss | 0.846059409 | 0.614258471 | ITGA3; ITGA3 | 37 | 17 |
| cg02008818 | 7.13E−10 | 3.03E−04 | −0.231773676 | 0.231773676 | loss | 0.741898158 | 0.510124482 | EIF5A2 | 37 | 3 |
| cg27238470 | 7.84E−10 | 3.33E−04 | −0.23170191 | 0.23170191 | loss | 0.719655492 | 0.487953582 | EDN2; EDN2 | 37 | 1 |
| cg23992493 | 8.47E−10 | 3.60E−04 | −0.231696103 | 0.231696103 | loss | 0.808873862 | 0.577177759 | | 37 | 17 |
| cg04810284 | 8.55E−08 | 3.63E−02 | −0.23167611 | 0.23167611 | loss | 0.943942092 | 0.712265982 | LINC01088 | 37 | 4 |
| cg13792913 | 7.13E−10 | 3.03E−04 | −0.231664166 | 0.231664166 | loss | 0.897968666 | 0.6663045 | | 37 | 11 |
| cg16186131 | 7.13E−10 | 3.03E−04 | −0.231643331 | 0.231643331 | loss | 0.823401913 | 0.591758582 | | 37 | 5 |
| cg26995224 | 4.97E−09 | 2.11E−03 | −0.231641947 | 0.231641947 | loss | 0.6067328 | 0.375090853 | KDM2B; KDM2B | 37 | 12 |
| cg10483275 | 7.13E−10 | 3.03E−04 | −0.231639847 | 0.231639847 | loss | 0.567550747 | 0.3359109 | CAMK2A; CAMK2A; CAMK2A; | 37 | 5 |
| | | | | | | | | CAMK2A | | |
| cg01891818 | 8.47E−10 | 3.60E−04 | −0.231620066 | 0.231620066 | loss | 0.538918613 | 0.307298547 | ICT1 | 37 | 17 |
| cg05465347 | 7.77E−10 | 3.30E−04 | −0.231614733 | 0.231614733 | loss | 0.736438951 | 0.504824218 | | 37 | 16 |
| cg15560801 | 7.13E−10 | 3.03E−04 | −0.231614315 | 0.231614315 | loss | 0.486681115 | 0.2550668 | | 37 | 1 |
| cg14038058 | 1.83E−09 | 7.75E−04 | −0.23155397 | 0.23155397 | loss | 0.308836366 | 0.077282396 | TRIM61; TRIM61 | 37 | 4 |
| cg19615684 | 8.47E−10 | 3.60E−04 | −0.231543902 | 0.231543902 | loss | 0.760911355 | 0.529367453 | CD9 | 37 | 12 |
| cg16936370 | 7.13E−10 | 3.03E−04 | −0.231516779 | 0.231516779 | loss | 0.727007396 | 0.495490618 | MMP19; MMP19; MMP19 | 37 | 12 |
| cg00407546 | 7.13E−10 | 3.03E−04 | −0.231510302 | 0.231510302 | loss | 0.402812725 | 0.171302422 | GATA3-AS1; GATA3- | 37 | 10 |
| | | | | | | | | AS1; GATA3-AS1; GATA3- | | |
| | | | | | | | | AS1; GATA3-AS1; GATA3- | | |
| | | | | | | | | AS1 | | |
| cg07548255 | 4.21E−09 | 1.79E−03 | −0.231471948 | 0.231471948 | loss | 0.574322077 | 0.342850129 | SH3RF3; SH3RF3- | 37 | 2 |
| | | | | | | | | AS1; SH3RF3-AS1 | | |
| cg01461856 | 8.47E−10 | 3.60E−04 | −0.231471218 | 0.231471218 | loss | 0.331630662 | 0.100159444 | | 37 | 17 |
| cg08355456 | 5.01E−08 | 2.13E−02 | −0.231456829 | 0.231456829 | loss | 0.496449011 | 0.264992182 | DOC2GP | 37 | 11 |
| cg08119295 | 1.12E−08 | 4.76E−03 | 0.231438473 | 0.231438473 | gain | 0.615347351 | 0.846785824 | TCEAL7 | 37 | X |
| cg26843498 | 6.35E−09 | 2.70E−03 | −0.231425704 | 0.231425704 | loss | 0.520422051 | 0.288996347 | | 37 | 19 |
| cg10556999 | 1.32E−08 | 5.59E−03 | −0.231424639 | 0.231424639 | loss | 0.922429115 | 0.691004476 | | 37 | 1 |
| cg18795786 | 5.84E−08 | 2.48E−02 | −0.231424195 | 0.231424195 | loss | 0.80010393 | 0.568679735 | CNTN1; CNTN1 | 37 | 12 |
| cg16137147 | 7.13E−10 | 3.03E−04 | −0.231418969 | 0.231418969 | loss | 0.278006243 | 0.046587274 | | 37 | 1 |
| cg04089788 | 7.13E−10 | 3.03E−04 | −0.231365641 | 0.231365641 | loss | 0.524790094 | 0.293424453 | PLXNB2 | 37 | 22 |
| cg27584013 | 7.13E−10 | 3.03E−04 | −0.231345231 | 0.231345231 | loss | 0.773150802 | 0.541805571 | | 37 | 1 |
| cg18476766 | 4.97E−09 | 2.11E−03 | −0.231343018 | 0.231343018 | loss | 0.8502442 | 0.618901182 | | 37 | 2 |
| cg25305153 | 9.54E−09 | 4.05E−03 | −0.231336376 | 0.231336376 | loss | 0.770489553 | 0.539153176 | ASB18 | 37 | 2 |
| cg22147968 | 7.13E−10 | 3.03E−04 | −0.231317872 | 0.231317872 | loss | 0.947009955 | 0.715692082 | CUX2 | 37 | 12 |
| cg19420604 | 1.54E−08 | 6.56E−03 | −0.231293381 | 0.231293381 | loss | 0.893319987 | 0.662026606 | MARCH10; MARCH10; MIR548W | 37 | 17 |
| cg19445044 | 3.10E−08 | 1.32E−02 | −0.231280176 | 0.231280176 | loss | 0.591613046 | 0.360332871 | | 37 | 12 |
| cg14997592 | 6.35E−09 | 2.70E−03 | −0.231271216 | 0.231271216 | loss | 0.713935151 | 0.482663935 | CRISP2; CRISP2; CRISP2; CRISP2; | 37 | 6 |
| | | | | | | | | CRISP2; CRISP2; CRISP2; | | |
| | | | | | | | | CRISP2; CRISP2; CR1SP2; | | |
| | | | | | | | | CRISP2; CRISP2 | | |
| cg19254328 | 7.13E−10 | 3.03E−04 | −0.231265634 | 0.231265634 | loss | 0.843862828 | 0.612597194 | CCDC37; LOC100506907; LOC100506907; | 37 | 3 |
| | | | | | | | | LOC100506907 | | |
| cg08168768 | 4.21E−09 | 1.79E−03 | −0.231259169 | 0.231259169 | loss | 0.648811187 | 0.417552018 | KIAA1210 | 37 | X |
| cg13040392 | 7.13E−10 | 3.03E−04 | −0.231256031 | 0.231256031 | loss | 0.627393113 | 0.396137082 | | 37 | 9 |
| cg05530317 | 1.32E−08 | 5.59E−03 | −0.231238525 | 0.231238525 | loss | 0.615024472 | 0.383785947 | EGFLAM; EGFLAM; EGFLAM; | 37 | 5 |
| | | | | | | | | EGFLAM | | |
| cg19699056 | 1.54E−09 | 6.54E−04 | −0.23121882 | 0.23121882 | loss | 0.762323085 | 0.531104265 | | 37 | 7 |
| cg21408915 | 7.13E−10 | 3.03E−04 | −0.231192052 | 0.231192052 | loss | 0.507174417 | 0.275982365 | | 37 | 11 |
| cg20278854 | 1.43E−08 | 6.06E−03 | −0.23119192 | 0.23119192 | loss | 0.573144896 | 0.341952976 | ZDHHC19 | 37 | 3 |
| cg25958766 | 2.78E−09 | 1.18E−03 | −0.231183055 | 0.231183055 | loss | 0.388077055 | 0.156894 | LINC00654; LINC00654 | 37 | 20 |
| cg17347634 | 2.35E−09 | 9.98E−04 | −0.231180266 | 0.231180266 | loss | 0.525873277 | 0.294693012 | CYP7B1 | 37 | 8 |
| cg07477034 | 7.13E−10 | 3.03E−04 | −0.231176173 | 0.231176173 | loss | 0.350180585 | 0.119004412 | HLF | 37 | 17 |
| cg25629768 | 1.96E−08 | 8.33E−03 | −0.23113564 | 0.23113564 | loss | 0.892216723 | 0.661081082 | TIMM13; LMNB2 | 37 | 19 |
| cg02162144 | 4.21E−09 | 1.79E−03 | −0.231037583 | 0.231037583 | loss | 0.79350483 | 0.562467247 | | 37 | 14 |
| cg15906733 | 7.13E−10 | 3.03E−04 | −0.231019024 | 0.231019024 | loss | 0.493098594 | 0.262079571 | | 37 | 17 |
| cg17285709 | 7.13E−10 | 3.03E−04 | −0.231017607 | 0.231017607 | loss | 0.757999577 | 0.526981971 | SPEG | 37 | 2 |
| cg05714155 | 8.47E−10 | 3.60E−04 | −0.230984583 | 0.230984583 | loss | 0.289795481 | 0.058810898 | | 37 | 2 |
| cg09289148 | 7.13E−10 | 3.03E−04 | −0.230907158 | 0.230907158 | loss | 0.583583711 | 0.352676553 | | 37 | 16 |
| cg22094845 | 4.57E−09 | 1.94E−03 | −0.230906834 | 0.230906834 | loss | 0.494050923 | 0.263144088 | ALPI | 37 | 2 |
| cg05063952 | 7.13E−10 | 3.03E−04 | −0.230881801 | 0.230881801 | loss | 0.491620813 | 0.260739012 | STAC2 | 37 | 17 |
| cg17971837 | 7.77E−10 | 3.30E−04 | −0.230878872 | 0.230878872 | loss | 0.680868372 | 0.4499895 | PVALB | 37 | 22 |
| cg20265528 | 1.01E−09 | 4.27E−04 | −0.230821086 | 0.230821086 | loss | 0.900775509 | 0.669954424 | | 37 | 2 |
| cg15772924 | 7.13E−10 | 3.03E−04 | −0.230816234 | 0.230816234 | loss | 0.441954923 | 0.211138688 | HOXC4; KOXC6; HOXC5 | 37 | 12 |
| cg04259907 | 9.23E−10 | 3.92E−04 | −0.230813201 | 0.230813201 | loss | 0.475295725 | 0.244482524 | LYPD3; LYPD3 | 37 | 19 |
| cg24810305 | 9.23E−10 | 3.92E−04 | −0.230807214 | 0.230807214 | loss | 0.507020796 | 0.276213582 | AMN | 37 | 14 |
| cg25641533 | 7.34E−08 | 3.12E−02 | −0.230799326 | 0.230799326 | loss | 0.758287079 | 0.527487753 | VWA7 | 37 | 6 |
| cg01565291 | 1.01E−09 | 4.27E−04 | −0.23079506 | 0.23079506 | loss | 0.706425325 | 0.475630265 | KIF26B | 37 | 1 |
| cg00786761 | 2.56E−09 | 1.09E−03 | −0.23078091 | 0.23078091 | loss | 0.944942334 | 0.714161424 | | 37 | 3 |
| cg19311244 | 7.13E−10 | 3.03E−04 | −0.230773745 | 0.230773745 | loss | 0.670716757 | 0.439943012 | | 37 | 4 |
| cg09630142 | 7.34E−08 | 3.12E−02 | −0.230763291 | 0.230763291 | loss | 0.762341874 | 0.531578582 | VWA7 | 37 | 6 |
| cg18472410 | 7.13E−10 | 3.03E−04 | −0.230761738 | 0.230761738 | loss | 0.767616826 | 0.536855088 | CCDC152 | 37 | 5 |
| cg24485927 | 7.13E−10 | 3.03E−04 | −0.230713918 | 0.230713918 | loss | 0.6003011 | 0.369587182 | LINC01115 | 37 | 2 |
| cg02537838 | 7.13E−10 | 3.03E−04 | −0.230699052 | 0.230699052 | loss | 0.896358681 | 0.665659629 | RBBP8NL; RBBP8NL | 37 | 20 |
| cg17335199 | 8.11E−09 | 3.44E−03 | −0.230678056 | 0.230678056 | loss | 0.916908015 | 0.686229959 | LINC01105 | 37 | 2 |
| cg06494185 | 1.54E−09 | 6.54E−04 | −0.230652682 | 0.230652682 | loss | 0.646079 | 0.415426318 | | 37 | 17 |
| cg21032567 | 7.13E−10 | 3.03E−04 | −0.230647608 | 0.230647608 | loss | 0.770937349 | 0.540289741 | LINC01115 | 37 | 2 |
| cg26312542 | 7.13E−10 | 3.03E−04 | −0.230622552 | 0.230622552 | loss | 0.947078664 | 0.716456112 | TEX12 | 37 | 11 |
| cg26276327 | 7.13E−10 | 3.03E−04 | −0.230586398 | 0.230586398 | loss | 0.352799991 | 0.122213592 | | 37 | 1 |
| cg26932623 | 9.23E−10 | 3.92E−04 | −0.230586317 | 0.230586317 | loss | 0.877843311 | 0.647255994 | | 37 | 1 |
| cg27592309 | 2.69E−08 | 1.14E−02 | −0.230585905 | 0.230585905 | loss | 0.66876167 | 0.438175765 | | 37 | 16 |
| cg13449377 | 1.30E−09 | 5.52E−04 | −0.230552744 | 0.230552744 | loss | 0.717470474 | 0.486917729 | DLX6-AS1 | 37 | 7 |
| cg14827832 | 2.30E−08 | 9.75E−03 | −0.230544303 | 0.230544303 | loss | 0.477639585 | 0.247095282 | | 37 | 17 |
| cg13238680 | 7.13E−10 | 3.03E−04 | −0.230518422 | 0.230518422 | loss | 0.81732554 | 0.586807118 | | 37 | 10 |
| cg00765765 | 3.02E−09 | 1.28E−03 | −0.23049795 | 0.23049795 | loss | 0.709066938 | 0.478568988 | | 37 | 2 |
| cg15924332 | 9.54E−09 | 4.05E−03 | −0.230482704 | 0.230482704 | loss | 0.520840951 | 0.290358247 | ITIHS; ITIHS | 37 | 10 |
| cg20272155 | 7.77E−10 | 3.30E−04 | −0.230461033 | 0.230461033 | loss | 0.500928815 | 0.270467782 | IFITM10; MOB2 | 37 | 11 |
| cg21672550 | 7.13E−10 | 3.03E−04 | −0.230432533 | 0.230432533 | loss | 0.785126509 | 0.554693976 | CNTFR; CNTFR; CNTFR; CNTFR- | 37 | 9 |
| | | | | | | | | AS1 | | |
| cg26719062 | 1.03E−08 | 4.39E−03 | −0.23042711 | 0.23042711 | loss | 0.520407857 | 0.289980747 | | 37 | 6 |
| cg24058386 | 7.13E−10 | 3.03E−04 | −0.230423195 | 0.230423195 | loss | 0.463528319 | 0.233105124 | C1orf61 | 37 | 1 |
| cg04476394 | 8.47E−10 | 3.60E−04 | −0.23041809 | 0.23041809 | loss | 0.469113713 | 0.238695624 | | 37 | 5 |
| cg08951051 | 1.83E−09 | 7.75E−04 | −0.230399391 | 0.230399391 | loss | 0.455567574 | 0.225168182 | | 37 | 16 |
| cg12453539 | 7.13E−10 | 3.03E−04 | −0.230397237 | 0.230397237 | loss | 0.405841719 | 0.175444482 | LRRN4 | 37 | 20 |
| cg23516451 | 7.13E−10 | 3.03E−04 | −0.230393233 | 0.230393233 | loss | 0.787008251 | 0.556615018 | OSMR; OSMR | 37 | 5 |
| cg11695684 | 1.54E−09 | 6.54E−04 | −0.230376229 | 0.230376229 | loss | 0.839896458 | 0.609520229 | CHRND; CHRND; CHRND; CHRND | 37 | 2 |
| cg01824138 | 1.10E−09 | 4.65E−04 | −0.230315727 | 0.230315727 | loss | 0.804030004 | 0.573714276 | CASZ1 | 37 | 1 |
| cg14540913 | 7.13E−10 | 3.03E−04 | −0.230293868 | 0.230293868 | loss | 0.595223474 | 0.364929606 | PRRX2 | 37 | 9 |
| cg03071221 | 7.13E−10 | 3.03E−04 | −0.230266778 | 0.230266778 | loss | 0.838129313 | 0.607862535 | | 37 | 11 |
| cg20335213 | 7.77E−10 | 3.30E−04 | −0.230263069 | 0.230263069 | loss | 0.720842151 | 0.490579082 | ARHGAP29 | 37 | 1 |
| cg06289725 | 7.77E−10 | 3.30E−04 | −0.230244308 | 0.230244308 | loss | 0.886872485 | 0.656628176 | CHRM1; CHRM1 | 37 | 11 |
| cg17596731 | 7.13E−10 | 3.03E−04 | −0.230212351 | 0.230212351 | loss | 0.878854351 | 0.648642 | | 37 | 5 |
| cg00586527 | 2.06E−09 | 8.77E−04 | −0.230195288 | 0.230195288 | loss | 0.865226451 | 0.635031163 | | 37 | 10 |
| cg24082680 | 8.47E−10 | 3.60E−04 | −0.230175209 | 0.230175209 | loss | 0.349507985 | 0.119332776 | ATG4C; ATG4C | 37 | 1 |
| cg11412129 | 7.13E−10 | 3.03E−04 | −0.230159873 | 0.230159873 | loss | 0.720299343 | 0.490139471 | | 37 | 1 |
| cg03213289 | 1.68E−09 | 7.12E−04 | −0.230154158 | 0.230154158 | loss | 0.419170185 | 0.189016026 | LOC63930 | 37 | 20 |
| cg20016673 | 1.32E−09 | 5.62E−04 | −0.230118571 | 0.230118571 | loss | 0.287642765 | 0.057524194 | NTNG1; NTNG1; NTNG1 | 37 | 1 |
| cg24743423 | 3.88E−09 | 1.65E−03 | −0.230113627 | 0.230113627 | loss | 0.500499157 | 0.270385529 | KAL1 | 37 | X |
| cg02837600 | 1.54E−09 | 6.54E−04 | −0.230097369 | 0.230097369 | loss | 0.607574145 | 0.377476776 | LOC642852 | 37 | 21 |
| cg23948843 | 7.13E−10 | 3.03E−04 | −0.230078032 | 0.230078032 | loss | 0.619931585 | 0.389853553 | | 37 | 2 |
| cg25521481 | 2.78E−09 | 1.18E−03 | −0.230061493 | 0.230061493 | loss | 0.510892428 | 0.280830935 | TTBK1 | 37 | 6 |
| cg06687091 | 7.13E−10 | 3.03E−04 | −0.230038541 | 0.230038541 | loss | 0.833148953 | 0.603110412 | RNF165; RNF165; RNF165 | 37 | 18 |
| cg24733126 | 1.30E−09 | 5.52E−04 | −0.229914086 | 0.229914086 | loss | 0.811493915 | 0.581579829 | SCUBE2; SCUBE2; MIR5691 | 37 | 11 |
| cg00645383 | 7.48E−09 | 3.18E−03 | −0.229904056 | 0.229904056 | loss | 0.717622221 | 0.487718165 | | 37 | 13 |
| cg17112828 | 1.89E−09 | 8.02E−04 | −0.22987423 | 0.22987423 | loss | 0.724886849 | 0.495012619 | LRRN2; LRRN2 | 37 | 1 |
| cg11757417 | 1.10E−09 | 4.65E−04 | −0.229864967 | 0.229864967 | loss | 0.849473702 | 0.619608735 | | 37 | 7 |
| cg04193820 | 9.23E−10 | 3.92E−04 | −0.229850518 | 0.229850518 | loss | 0.363752838 | 0.133902319 | RNF135; RNF135; RNF135 | 37 | 17 |
| cg03720745 | 9.23E−10 | 3.92E−04 | −0.22981975 | 0.22981975 | loss | 0.566617185 | 0.336797435 | | 37 | 8 |
| cg15687395 | 2.56E−09 | 1.09E−03 | −0.229800915 | 0.229800915 | loss | 0.744277574 | 0.514476659 | | 37 | 5 |
| cg26317834 | 7.13E−10 | 3.03E−04 | −0.22974924 | 0.22974924 | loss | 0.715990087 | 0.486240847 | SMTNL2 | 37 | 17 |
| cg11133013 | 7.13E−10 | 3.03E−04 | −0.229726607 | 0.229726607 | loss | 0.882456213 | 0.652729606 | FAM83C; FAM83C | 37 | 20 |
| cg13921251 | 7.77E−10 | 3.30E−04 | −0.229681067 | 0.229581067 | loss | 0.800895885 | 0.571214818 | | 37 | 9 |
| cg27005487 | 7.13E−10 | 3.03E−04 | −0.229617473 | 0.229617473 | loss | 0.768349943 | 0.538732471 | TBX2 | 37 | 17 |
| cg24856064 | 2.78E−09 | 1.18E−03 | −0.229614752 | 0.229614752 | loss | 0.353217175 | 0.123602423 | TEX26; TEX26-AS1; TEX26- | 37 | 13 |
| | | | | | | | | AS1 | | |
| cg23422156 | 7.13E−10 | 3.03E−04 | −0.229572267 | 0.229572267 | loss | 0.277740058 | 0.048167791 | CCDC42B | 37 | 12 |
| cg24827726 | 7.77E−10 | 3.30E−04 | −0.229557527 | 0.229557527 | loss | 0.729817151 | 0.500259624 | GRIP2 | 37 | 3 |
| cg22291359 | 8.47E−10 | 3.60E−04 | −0.229555626 | 0.229555626 | loss | 0.359240945 | 0.129685319 | PIGL; NCOR1 | 37 | 17 |
| cg08636191 | 2.78E−09 | 1.18E−03 | −0.229553466 | 0.229553466 | loss | 0.776165977 | 0.546612512 | | 37 | 7 |
| cg08072539 | 7.13E−10 | 3.03E−04 | −0.229549071 | 0.229549071 | loss | 0.853833036 | 0.624283965 | TDRP | 37 | 8 |
| cg07903023 | 7.13E−10 | 3.03E−04 | −0.229538782 | 0.229538782 | loss | 0.610591753 | 0.381052971 | KLHL30 | 37 | 2 |
| cg26422151 | 7.13E−10 | 3.03E−04 | −0.229536724 | 0.229536724 | loss | 0.387309636 | 0.157772912 | | 37 | 10 |
| cg08102414 | 1.83E−09 | 7.75E−04 | −0.229534296 | 0.229534296 | loss | 0.593564508 | 0.364030212 | | 37 | 4 |
| cg05622003 | 7.13E−10 | 3.03E−04 | −0.22951622 | 0.22951622 | loss | 0.685306543 | 0.455790324 | | 37 | 5 |
| cg02692893 | 1.81E−08 | 7.69E−03 | −0.229489549 | 0.229489549 | loss | 0.641669555 | 0.412180106 | ROPN1 | 37 | 3 |
| cg03054684 | 8.80E−09 | 3.74E−03 | −0.229472273 | 0.229472273 | loss | 0.441880832 | 0.212408559 | MOBP; MOBP; MOBP; MOBP; | 37 | 3 |
| | | | | | | | | MOBP; MOBP; MOBP | | |
| cg01381795 | 7.13E−10 | 3.03E−04 | −0.229459427 | 0.229459427 | loss | 0.775272321 | 0.545812894 | CACNG6; CACNG6; CACNG6; | 37 | 19 |
| | | | | | | | | CACNG6 | | |
| cg19537719 | 3.57E−09 | 1.52E−03 | −0.229455252 | 0.229455252 | loss | 0.317242567 | 0.087787315 | ANK1; ANK1; ANK1; ANK1; | 37 | 8 |
| | | | | | | | | ANK1 | | |
| cg20265748 | 7.13E−10 | 3.03E−04 | −0.229448036 | 0.229448036 | loss | 0.764445736 | 0.5349977 | PTPN21 | 37 | 14 |
| cg13698883 | 1.42E−09 | 6.01E−04 | −0.229363435 | 0.229383435 | loss | 0.74024557 | 0.510862135 | IGFBPL1 | 37 | 9 |
| cg15712226 | 7.13E−10 | 3.03E−04 | −0.229348042 | 0.229348042 | loss | 0.273987015 | 0.044638974 | SNRPF | 37 | 12 |
| cg13839439 | 7.13E−10 | 3.03E−04 | −0.229338424 | 0.229338424 | loss | 0.705774494 | 0.476436071 | CISTR-ACT; CISTR-ACT | 37 | 12 |
| cg13726191 | 2.48E−08 | 1.05E−02 | −0.229330078 | 0.229330078 | loss | 0.485147555 | 0.256817476 | FGFBP1 | 37 | 4 |
| cg06324373 | 2.78E−09 | 1.18E−03 | −0.229327534 | 0.229327534 | loss | 0.387938987 | 0.158611453 | CRTAC1; CRTAC1 | 37 | 10 |
| cg14635586 | 6.89E−09 | 2.93E−03 | −0.229325103 | 0.229325103 | loss | 0.666161385 | 0.436836282 | | 37 | 6 |
| cg26478107 | 7.13E−10 | 3.03E−04 | −0.229293823 | 0.229293823 | loss | 0.391470953 | 0.162177129 | | 37 | 6 |
| cg02455471 | 7.13E−10 | 3.03E−04 | −0.229281073 | 0.229281073 | loss | 0.913478149 | 0.684197076 | | 37 | 5 |
| cg25201363 | 7.13E−10 | 3.03E−04 | −0.22927271 | 0.22927271 | loss | 0.528528757 | 0.299256047 | LRPAP1 | 37 | 4 |
| cg21686577 | 7.13E−10 | 3.03E−04 | −0.229232518 | 0.229232518 | loss | 0.401305336 | 0.172072818 | SRRM3 | 37 | 7 |
| cg23173688 | 8.47E−10 | 3.60E−04 | −0.229177159 | 0.229177159 | loss | 0.43492273 | 0.205745571 | | 37 | 1 |
| cg16611278 | 1.30E−09 | 5.52E−04 | −0.229177127 | 0.229177127 | loss | 0.911473004 | 0.682295876 | DTX1 | 37 | 12 |
| cg00118978 | 7.13E−10 | 3.03E−04 | −0.229176034 | 0.229176034 | loss | 0.369505877 | 0.140329843 | | 37 | 3 |
| cg07009504 | 7.13E−10 | 3.03E−04 | −0.229170753 | 0.229170753 | loss | 0.573100006 | 0.343929253 | | 37 | 13 |
| cg15768933 | 7.13E−10 | 3.03E−04 | −0.229158953 | 0.229158953 | loss | 0.889746736 | 0.660587782 | ECEL1 | 37 | 2 |
| cg09322513 | 9.23E−10 | 3.92E−04 | −0.229141237 | 0.229141237 | loss | 0.386485319 | 0.157344082 | LINC00922 | 37 | 16 |
| cg00926894 | 4.57E−09 | 1.94E−03 | −0.229124284 | 0.229124284 | loss | 0.60071836 | 0.371594076 | RGN; RGN; RGN; RGN | 37 | X |
| cg16625671 | 1.99E−09 | 8.43E−04 | −0.229106026 | 0.229106026 | loss | 0.877188296 | 0.648082271 | | 37 | 5 |
| cg02475474 | 5.41E−08 | 2.30E−02 | −0.22902091 | 0.22902091 | loss | 0.844272045 | 0.615251135 | MOG; MOG; MOG; MOG; | 37 | 6 |
| | | | | | | | | MOG; MOG; MOG; MOG; | | |
| | | | | | | | | MOG | | |
| cg13912311 | 1.19E−09 | 5.07E−04 | −0.229007878 | 0.229007878 | loss | 0.609976208 | 0.380968329 | NR5A1 | 37 | 9 |
| cg18244845 | 7.84E−10 | 3.33E−04 | −0.228992705 | 0.228992705 | loss | 0.730493535 | 0.501500829 | RFX2; RFX2 | 37 | 19 |
| cg11421509 | 1.22E−08 | 5.16E−03 | −0.228969786 | 0.228969786 | loss | 0.460311445 | 0.231341659 | | 37 | 8 |
| cg25397029 | 8.11E−09 | 3.44E−03 | −0.228964303 | 0.228964303 | loss | 0.638120985 | 0.409156682 | ANKRD2; ANKRD2 | 37 | 10 |
| cg22130008 | 9.23E−10 | 3.92E−04 | −0.22896192 | 0.22896192 | loss | 0.445973943 | 0.217012024 | | 37 | 4 |
| cg06530441 | 8.11E−09 | 3.44E−03 | −0.228958923 | 0.228958923 | loss | 0.361848292 | 0.132889369 | CPT1B; CPT1B; CPT1B; CPT1B; | 37 | 22 |
| | | | | | | | | CPT1B; CPT1B; CPT1B; | | |
| | | | | | | | | CPT1B; CPT1B; CPT1B; CPT1B; | | |
| | | | | | | | | CPT1B; CPT1B; CHKB- | | |
| | | | | | | | | CPT1B | | |
| cg26550330 | 7.13E−10 | 3.03E−04 | −0.228938148 | 0.228938148 | loss | 0.704861036 | 0.475922888 | OBSCN; OBSCN; OBSCN; C1orf145; | 37 | 1 |
| | | | | | | | | C1orf145 | | |
| cg13870426 | 7.84E−10 | 3.33E−04 | −0.228935584 | 0.228935584 | loss | 0.822593225 | 0.593657641 | | 37 | 17 |
| cg04090347 | 4.97E−09 | 2.11E−03 | −0.228883652 | 0.228883652 | loss | 0.617950423 | 0.389066771 | | 37 | 21 |
| cg19813226 | 7.13E−10 | 3.03E−04 | −0.228883114 | 0.228883114 | loss | 0.357206038 | 0.128322924 | | 37 | 1 |
| cg16263848 | 1.54E−09 | 6.54E−04 | −0.228871468 | 0.228871468 | loss | 0.643104168 | 0.4142327 | NTN1 | 37 | 17 |
| cg16985708 | 7.13E−10 | 3.03E−04 | −0.228864207 | 0.228864207 | loss | 0.45938086 | 0.230516653 | | 37 | 7 |
| cg07253829 | 8.47E−10 | 3.60E−04 | −0.228827702 | 0.228827702 | loss | 0.419107196 | 0.190279494 | DPPA4; DPPA4 | 37 | 3 |
| cg24933709 | 7.13E−10 | 3.03E−04 | −0.228804266 | 0.228804266 | loss | 0.387716081 | 0.158911815 | | 37 | 7 |
| cg15161667 | 7.13E−10 | 3.03E−04 | −0.228798284 | 0.228798284 | loss | 0.753850096 | 0.525051812 | LRP4 | 37 | 11 |
| cg11737172 | 1.16E−07 | 4.91E−02 | −0.228781217 | 0.228781217 | loss | 0.460887806 | 0.232106588 | KBTBD11 | 37 | 8 |
| cg18569141 | 7.13E−10 | 3.03E−04 | −0.22876441 | 0.22876441 | loss | 0.344330498 | 0.115566088 | SEMA4B | 37 | 15 |
| cg27364650 | 1.67E−08 | 7.10E−03 | −0.228759599 | 0.228759599 | loss | 0.420646187 | 0.191886588 | | 37 | 3 |
| cg08182193 | 3.02E−09 | 1.28E−03 | −0.228720992 | 0.228720992 | loss | 0.458449845 | 0.229728853 | CRIP2; CRIP2; CRIP2; CRIP2; | 37 | 14 |
| | | | | | | | | CRIP2; CRIP2; CRIP2; CRIP2 | | |
| cg17396305 | 8.80E−09 | 3.74E−03 | −0.228686082 | 0.228686082 | loss | 0.551073311 | 0.322387229 | | 37 | 6 |
| cg08178168 | 7.13E−10 | 3.03E−04 | −0.228682873 | 0.228682873 | loss | 0.528787238 | 0.300104365 | | 37 | 22 |
| cg02116131 | 7.13E−10 | 3.03E−04 | −0.228648575 | 0.228648575 | loss | 0.765252275 | 0.5366037 | | 37 | 17 |
| cg26582789 | 3.28E−09 | 1.39E−03 | −0.228644954 | 0.228644954 | loss | 0.463013196 | 0.234368242 | MAP3K15 | 37 | X |
| cg05068206 | 5.01E−08 | 2.13E−02 | −0.228628507 | 0.228628507 | loss | 0.412803809 | 0.184175302 | | 37 | 6 |
| cg21293216 | 7.13E−10 | 3.03E−04 | −0.22861741 | 0.22861741 | loss | 0.833310951 | 0.604693541 | IL22RA1; IL22RA1 | 37 | 1 |
| cg14325661 | 7.13E−10 | 3.03E−04 | −0.228607289 | 0.228607289 | loss | 0.659317936 | 0.430710647 | TRPM4; TRPM4 | 37 | 19 |
| cg11143671 | 9.23E−10 | 3.92E−04 | −0.228580812 | 0.228580812 | loss | 0.439786794 | 0.211205982 | KIAA1614 | 37 | 1 |
| cg19227924 | 7.13E−10 | 3.03E−04 | −0.228540483 | 0.228540483 | loss | 0.378581158 | 0.150040675 | | 37 | 17 |
| cg14413050 | 3.02E−09 | 1.28E−03 | −0.228518704 | 0.228518704 | loss | 0.831751298 | 0.603232594 | | 37 | 13 |
| cg07552868 | 1.01E−09 | 4.27E−04 | −0.228510357 | 0.228510357 | loss | 0.797629434 | 0.569119076 | PDE4DIP; PDE4DIP; PDE4DIP; | 37 | 1 |
| | | | | | | | | PDE4DIP; PDE4DIP; PDE4DIP; | | |
| | | | | | | | | NBPF9; LOC100288142; | | |
| | | | | | | | | PDE4DIP; PDE4DIP | | |
| cg02802029 | 1.83E−09 | 7.75E−04 | −0.228484622 | 0.228484622 | loss | 0.540763628 | 0.312279006 | PLOD2; PLOD2 | 37 | 3 |
| cg05854695 | 1.42E−09 | 6.01E−04 | −0.228481376 | 0.228481376 | loss | 0.830762158 | 0.602280782 | TRPC3; TRPC3; TRPC3 | 37 | 4 |
| cg08784911 | 9.23E−10 | 3.92E−04 | −0.22845637 | 0.22845637 | loss | 0.551940117 | 0.323483747 | | 37 | 1 |
| cg26416971 | 8.47E−10 | 3.60E−04 | −0.228448245 | 0.228448245 | loss | 0.445868562 | 0.217420318 | CKMT2; CKMT2; CKMT2; RNU5E- | 37 | 5 |
| | | | | | | | | 1; NU5E-1; RNU5D-1 | | |
| cg02467858 | 2.16E−09 | 9.17E−04 | −0.228434437 | 0.228434437 | loss | 0.721311066 | 0.492876629 | TTC23; TTC23; TTC23; TTC23; | 37 | 15 |
| | | | | | | | | TTC23; TTC23; LRRC28; | | |
| | | | | | | | | TTC23; LRRC28 | | |
| cg10130718 | 8.47E−10 | 3.60E−04 | −0.228379582 | 0.228379582 | loss | 0.924334147 | 0.695954565 | DMRTB1 | 37 | 1 |
| cg00019841 | 8.47E−10 | 3.60E−04 | −0.228371656 | 0.228371656 | loss | 0.908476126 | 0.680104471 | MAPK4 | 37 | 18 |
| cg21944224 | 7.13E−10 | 3.03E−04 | −0.228362405 | 0.228362405 | loss | 0.424185381 | 0.195822976 | | 37 | 3 |
| cg23534245 | 3.57E−09 | 1.52E−03 | −0.22833861 | 0.22833861 | loss | 0.593997045 | 0.365658435 | BMP2 | 37 | 20 |
| cg13096592 | 9.23E−10 | 3.92E−04 | −0.228329673 | 0.228329673 | loss | 0.608328543 | 0.379998871 | | 37 | 8 |
| cg01244934 | 9.23E−10 | 3.92E−04 | −0.228291112 | 0.228291112 | loss | 0.877267847 | 0.648976735 | | 37 | 1 |
| cg14218621 | 7.13E−10 | 3.03E−04 | −0.228273442 | 0.228273442 | loss | 0.760188989 | 0.531915547 | C9orf171; C9orf171 | 37 | 9 |
| cg14645861 | 7.77E−10 | 3.30E−04 | −0.228272848 | 0.228272848 | loss | 0.905114189 | 0.676841341 | | 37 | 6 |
| cg13627197 | 1.54E−09 | 6.54E−04 | −0.228242668 | 0.228242668 | loss | 0.706572474 | 0.478329806 | PIWIL3; PIWIL3; TOP1P2; PIWIL3; | 37 | 22 |
| | | | | | | | | PIWIL3 | | |
| cg24932585 | 7.13E−10 | 3.03E−04 | −0.228220887 | 0.228220887 | loss | 0.387330134 | 0.159109247 | | 37 | 19 |
| cg12927641 | 1.54E−09 | 6.54E−04 | −0.228183701 | 0.228183701 | loss | 0.923712536 | 0.695528835 | | 37 | 6 |
| cg02737384 | 7.77E−10 | 3.30E−04 | −0.228181369 | 0.228181369 | loss | 0.361808528 | 0.133627159 | RASSF4 | 37 | 10 |
| cg18973515 | 7.13E−10 | 3.03E−04 | −0.228126857 | 0.228126857 | loss | 0.438274245 | 0.210147388 | IGDCC4 | 37 | 15 |
| cg15676707 | 1.30E−09 | 5.52E−04 | −0.228100123 | 0.228100123 | loss | 0.534010353 | 0.305910229 | | 37 | 6 |
| cg07733098 | 7.13E−10 | 3.03E−04 | −0.228085969 | 0.228085969 | loss | 0.440626228 | 0.212540259 | NRXN2; NRXN2 | 37 | 11 |
| cg00738772 | 7.13E−10 | 3.03E−04 | −0.228065688 | 0.228065688 | loss | 0.579949311 | 0.351883624 | | 37 | 7 |
| cg26057784 | 5.85E−09 | 2.49E−03 | −0.228061309 | 0.228061309 | loss | 0.569338962 | 0.341277653 | DPF3; DPF3; DPF3 | 37 | 14 |
| cg11191337 | 8.47E−10 | 3.60E−04 | −0.228061239 | 0.228061239 | loss | 0.823867257 | 0.595806018 | PLTP; PLTP; PLTP | 37 | 20 |
| cg04661709 | 7.13E−10 | 3.03E−04 | −0.228049065 | 0.228049065 | loss | 0.663295683 | 0.435246618 | DAB2IP | 37 | 9 |
| cg18279134 | 7.84E−10 | 3.33E−04 | −0.228028865 | 0.228028865 | loss | 0.809358506 | 0.581329641 | FAM131C | 37 | 1 |
| cg12284521 | 1.19E−09 | 5.07E−04 | −0.228021339 | 0.228021339 | loss | 0.699694351 | 0.471673012 | GRM4; GRM4; GRM4; GRM4; | 37 | 6 |
| | | | | | | | | GRM4; GRM4; GRM4 | | |
| cg14283082 | 7.13E−10 | 3.03E−04 | −0.227994733 | 0.227994733 | loss | 0.586938151 | 0.358943418 | | 37 | 8 |
| cg27118929 | 1.73E−09 | 7.34E−04 | −0.227982376 | 0.227982376 | loss | 0.76323817 | 0.535255794 | NOX5; NOX5; NOX5; MIR548H4; | 37 | 15 |
| | | | | | | | | NOX5; NOX5 | | |
| cg00788760 | 7.13E−10 | 3.03E−04 | −0.227963454 | 0.227963454 | loss | 0.566374677 | 0.338411224 | KIAA1644 | 37 | 22 |
| cg04396112 | 8.11E−09 | 3.44E−03 | −0.227940376 | 0.227940376 | loss | 0.809832523 | 0.581892147 | MAD1L1; MAD1L1; MAD1L1 | 37 | 7 |
| cg04898026 | 7.77E−10 | 3.30E−04 | −0.227934615 | 0.227934615 | loss | 0.564077421 | 0.336142806 | SSPO | 37 | 7 |
| cg15555527 | 1.30E−09 | 5.52E−04 | −0.22791628 | 0.22791628 | loss | 0.934782062 | 0.706865782 | ANTXR2; ANTXR2; ANTXR2 | 37 | 4 |
| cg10776186 | 1.68E−09 | 7.12E−04 | −0.22791053 | 0.22791053 | loss | 0.673802283 | 0.445891753 | NUPL1; NUPL1 | 37 | 13 |
| cg27655214 | 8.46E−10 | 3.59E−04 | −0.227848241 | 0.227848241 | loss | 0.277279813 | 0.049431572 | | 37 | 12 |
| cg26316885 | 3.02E−09 | 1.28E−03 | −0.227816506 | 0.227816506 | loss | 0.453001936 | 0.225185429 | ICAM5 | 37 | 19 |
| cg12070646 | 7.13E−10 | 3.03E−04 | −0.227812668 | 0.227812668 | loss | 0.925047721 | 0.697235053 | VAX2 | 37 | 2 |
| cg10399005 | 9.23E−10 | 3.92E−04 | −0.227803104 | 0.227803104 | loss | 0.87857254 | 0.650769435 | | 37 | 14 |
| cg22219795 | 7.13E−10 | 3.03E−04 | −0.227774873 | 0.227774873 | loss | 0.964844849 | 0.737069976 | C3orf56 | 37 | 3 |
| cg18833933 | 7.13E−10 | 3.03E−04 | −0.227730452 | 0.227730452 | loss | 0.558641475 | 0.330911024 | LRCOL1 | 37 | 12 |
| cg27038634 | 7.13E−10 | 3.03E−04 | −0.227690591 | 0.227690591 | loss | 0.455349474 | 0.227658882 | | 37 | 8 |
| cg02375506 | 7.13E−10 | 3.03E−04 | −0.227652087 | 0.227652087 | loss | 0.87125394 | 0.643601853 | MPPED1 | 37 | 22 |
| cg06219995 | 7.13E−10 | 3.03E−04 | −0.227642491 | 0.227642491 | loss | 0.450244991 | 0.2226025 | CLCNKB; CLCNKB | 37 | 1 |
| cg24408057 | 7.13E−10 | 3.03E−04 | −0.227613969 | 0.227613969 | loss | 0.716274881 | 0.488660912 | SHD | 37 | 19 |
| cg22062555 | 7.13E−10 | 3.03E−04 | −0.227607567 | 0.227607567 | loss | 0.754199885 | 0.526592318 | NKPD1 | 37 | 19 |
| cg20547777 | 2.48E−08 | 1.05E−02 | −0.227581317 | 0.227581317 | loss | 0.703367675 | 0.475786359 | EXT1 | 37 | 8 |
| cg09049251 | 7.77E−10 | 3.30E−04 | −0.227563523 | 0.227563523 | loss | 0.352326368 | 0.124762845 | FAIM2 | 37 | 12 |
| cg17529386 | 4.97E−09 | 2.11E−03 | −0.227518547 | 0.227518547 | loss | 0.714499864 | 0.486981318 | | 37 | 6 |
| cg15645685 | 2.35E−09 | 9.98E−04 | −0.227462511 | 0.227462511 | loss | 0.749276628 | 0.521814118 | PBX4; PBX4 | 37 | 19 |
| cg05298458 | 7.13E−10 | 3.03E−04 | −0.227461784 | 0.227461784 | loss | 0.461279966 | 0.233818182 | TBX1; TBX1; TBX1 | 37 | 22 |
| cg05973455 | 7.13E−10 | 3.03E−04 | −0.227445759 | 0.227445759 | loss | 0.672378842 | 0.444933082 | | 37 | 4 |
| cg17841765 | 1.19E−09 | 5.07E−04 | −0.227412641 | 0.227412641 | loss | 0.557111817 | 0.329699176 | | 37 | 1 |
| cg27128734 | 1.68E−09 | 7.12E−04 | −0.227411658 | 0.227411658 | loss | 0.849047911 | 0.621636253 | KLK4 | 37 | 19 |
| cg15169829 | 7.77E−10 | 3.30E−04 | −0.227381859 | 0.227381859 | loss | 0.528988236 | 0.301606376 | TMPRSS12 | 37 | 12 |
| cg25569480 | 3.02E−09 | 1.28E−03 | −0.227381007 | 0.227381007 | loss | 0.707191025 | 0.479810018 | GTF2A1L; STON1- | 37 | 2 |
| | | | | | | | | GTF2A1L; STON1- | | |
| | | | | | | | | GTF2A1L; GTF2A1L; STON1- | | |
| | | | | | | | | GTF2A1L | | |
| cg14126466 | 2.35E−09 | 9.98E−04 | −0.22738 | 0.22738 | loss | 0.562143558 | 0.334763559 | TBX5; TBX5; TBX5 | 37 | 12 |
| cg21506565 | 1.30E−09 | 5.52E−04 | −0.227350515 | 0.227350515 | loss | 0.834669957 | 0.607319441 | C6orf25; C6orf25; C6orf25; | 37 | 6 |
| | | | | | | | | C6orf25; C6orf25; C6orf25 | | |
| cg03177666 | 1.30E−09 | 5.52E−04 | −0.227343197 | 0.227343197 | loss | 0.763038738 | 0.535695541 | | 37 | 16 |
| cg13808198 | 2.56E−09 | 1.09E−03 | −0.227328313 | 0.227328313 | loss | 0.80916256 | 0.581834247 | ERRFI1 | 37 | 1 |
| cg05473289 | 7.13E−10 | 3.03E−04 | −0.22732721 | 0.22732721 | loss | 0.690027292 | 0.462700082 | TNXB | 37 | 6 |
| cg01751047 | 7.77E−10 | 3.30E−04 | −0.227317439 | 0.227317439 | loss | 0.694084251 | 0.466766812 | CDH15 | 37 | 16 |
| cg06600725 | 1.01E−09 | 4.27E−04 | −0.227265936 | 0.227265936 | loss | 0.346395253 | 0.119129316 | | 37 | 12 |
| cg08853304 | 7.13E−10 | 3.03E−04 | −0.227257152 | 0.227257152 | loss | 0.749702028 | 0.522444876 | | 37 | 5 |
| cg13683667 | 7.13E−10 | 3.03E−04 | −0.227239879 | 0.227239879 | loss | 0.981110374 | 0.753870494 | | 37 | 1 |
| cg05339633 | 7.13E−10 | 3.03E−04 | −0.227233561 | 0.227233561 | loss | 0.602212438 | 0.374978876 | MAP6D1 | 37 | 3 |
| cg06179011 | 9.54E−09 | 4.05E−03 | −0.227214161 | 0.227214161 | loss | 0.424585809 | 0.197371649 | TSPAN4; TSPAN4; TSPAN4; | 37 | 11 |
| | | | | | | | | TSPAN4; TSPAN4; TSPAN4; | | |
| | | | | | | | | TSPAN4; POLR2L | | |
| cg06961278 | 7.13E−10 | 3.03E−04 | −0.227213367 | 0.227213367 | loss | 0.784639196 | 0.557425829 | | 37 | 2 |
| cg25447164 | 7.92E−08 | 3.36E−02 | −0.227193082 | 0.227193082 | loss | 0.52767217 | 0.300479088 | | 37 | 17 |
| cg07516196 | 3.57E−09 | 1.52E−03 | −0.227168082 | 0.227168082 | loss | 0.61198257 | 0.384814488 | RNF39; KNF39 | 37 | 6 |
| cg18349298 | 1.68E−09 | 7.12E−04 | −0.227161598 | 0.227161598 | loss | 0.398099745 | 0.170938148 | RARRES1; RARRES1 | 37 | 3 |
| cg16212298 | 1.01E−09 | 4.27E−04 | −0.227156482 | 0.227156482 | loss | 0.939880064 | 0.712723582 | LRRC4B | 37 | 19 |
| cg04208124 | 7.13E−10 | 3.03E−04 | −0.227147132 | 0.227147132 | loss | 0.839284938 | 0.612137806 | AHNAK | 37 | 11 |
| cg26313888 | 7.13E−10 | 3.03E−04 | −0.227132576 | 0.227132576 | loss | 0.865521664 | 0.638389088 | LOC541473; LOC100101148 | 37 | 7 |
| cg19878482 | 3.97E−08 | 1.69E−02 | −0.227115516 | 0.227115516 | loss | 0.492073687 | 0.264958171 | MROH6; MROH6 | 37 | 8 |
| cg20322093 | 7.84E−10 | 3.33E−04 | −0.227110661 | 0.227110661 | loss | 0.476198802 | 0.249088141 | OTOP3; OTOP3 | 37 | 17 |
| cg09173348 | 1.68E−09 | 7.12E−04 | −0.227083294 | 0.227083294 | loss | 0.530148606 | 0.303065312 | ARL5C; ARL5C | 37 | 17 |
| cg11182874 | 1.43E−08 | 6.06E−03 | −0.227066463 | 0.227066463 | loss | 0.860328492 | 0.633262029 | | 37 | 8 |
| cg14848685 | 9.22E−08 | 3.91E−02 | −0.227034543 | 0.227034543 | loss | 0.928104413 | 0.701069871 | KIAA1244 | 37 | 6 |
| cg00496961 | 9.94E−08 | 4.22E−02 | −0.226950043 | 0.226950043 | loss | 0.548154702 | 0.321204659 | | 37 | 11 |
| cg07944445 | 1.01E−09 | 4.27E−04 | −0.226891079 | 0.226891079 | loss | 0.563618491 | 0.336727412 | | 37 | 7 |
| cg19236794 | 1.01E−09 | 4.27E−04 | −0.22687087 | 0.22687087 | loss | 0.399251653 | 0.172380782 | FAM132A | 37 | 1 |
| cg01511806 | 7.13E−10 | 3.03E−04 | −0.226858258 | 0.226858258 | loss | 0.837050917 | 0.610192659 | | 37 | 16 |
| cg25762706 | 2.16E−09 | 9.17E−04 | −0.226803373 | 0.226803373 | loss | 0.724872396 | 0.498069024 | STMN4; STMN4; STMN4; STMN4; | 37 | 8 |
| | | | | | | | | STMN4; STMN4; STMN4; | | |
| | | | | | | | | STMN4 | | |
| cg07875335 | 7.77E−10 | 3.30E−04 | −0.226792662 | 0.226792662 | loss | 0.789682657 | 0.562889994 | GTF2A1L; STON1- | 37 | 2 |
| | | | | | | | | GTF2A1L; STON1- | | |
| | | | | | | | | GTF2A1L; GTF2A1L; STON1- | | |
| | | | | | | | | GTF2A1L | | |
| cg13551783 | 9.23E−10 | 3.92E−04 | −0.226792434 | 0.226792434 | loss | 0.575379528 | 0.348587094 | CTRC | 37 | 1 |
| cg04231541 | 1.19E−09 | 5.07E−04 | −0.226789767 | 0.226789767 | loss | 0.973217879 | 0.746428112 | PRSS41 | 37 | 16 |
| cg25136631 | 2.16E−09 | 9.17E−04 | −0.22677762 | 0.22677762 | loss | 0.765492238 | 0.538714618 | HLA-DQB2 | 37 | 6 |
| cg13762474 | 1.19E−09 | 5.07E−04 | −0.226774243 | 0.226774243 | loss | 0.769866813 | 0.543092571 | PLA2G4D | 37 | 15 |
| cg26373716 | 5.01E−08 | 2.13E−02 | −0.22674814 | 0.22674814 | loss | 0.642614681 | 0.415866541 | FAM150A | 37 | 8 |
| cg08650934 | 7.13E−10 | 3.03E−04 | −0.226743245 | 0.226743245 | loss | 0.876539751 | 0.649796506 | | 37 | 21 |
| cg21894287 | 1.68E−09 | 7.12E−04 | −0.226689108 | 0.226689108 | loss | 0.776570596 | 0.549881488 | | 37 | 18 |
| cg20704654 | 5.84E−08 | 2.48E−02 | −0.226676665 | 0.226676665 | loss | 0.776654653 | 0.549977988 | REM1; LINC00028 | 37 | 20 |
| cg04897644 | 9.23E−10 | 3.92E−04 | −0.226672181 | 0.226672181 | loss | 0.634880792 | 0.408208612 | PKLR; PKLR | 37 | 1 |
| cg08977611 | 7.13E−10 | 3.03E−04 | −0.226624244 | 0.226624244 | loss | 0.518653726 | 0.292029482 | | 37 | 20 |
| cg02063817 | 1.19E−09 | 5.07E−04 | −0.226559737 | 0.226559737 | loss | 0.533233513 | 0.306673776 | | 37 | 7 |
| cg03300078 | 1.01E−09 | 4.27E−04 | −0.226545293 | 0.226545293 | loss | 0.475597475 | 0.249052182 | IFITM10; MOB2 | 37 | 11 |
| cg10841756 | 1.10E−09 | 4.65E−04 | −0.226542642 | 0.226542642 | loss | 0.812857072 | 0.586314429 | PVRL4; PVRL4 | 37 | 1 |
| cg08418111 | 2.35E−09 | 9.98E−04 | −0.226523479 | 0.226523479 | loss | 0.594057779 | 0.3675343 | LDHC; LDHC | 37 | 11 |
| cg04768479 | 1.68E−09 | 7.12E−04 | −0.226517166 | 0.226517166 | loss | 0.514407108 | 0.287889941 | | 37 | 6 |
| cg24771017 | 7.84E−10 | 3.33E−04 | −0.226512694 | 0.226512694 | loss | 0.975582694 | 0.74907 | CACNA1G; CACNA1G; CACNA1G; | 37 | 17 |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; CACNA1G; | | |
| | | | | | | | | CACNA1G; CACNA1G; | | |
| | | | | | | | | CACNA1G; CACNA1G; | | |
| | | | | | | | | CACNA1G; CACNA1G | | |
| cg13300939 | 1.19E−09 | 5.07E−04 | −0.226509266 | 0.226509266 | loss | 0.877897519 | 0.651388253 | GPR37L1 | 37 | 1 |
| cg23182880 | 1.42E−09 | 6.01E−04 | −0.22648668 | 0.22648668 | loss | 0.936996357 | 0.710509676 | NRXN2; NRXN2; NRXN2 | 37 | 11 |
| cg06777215 | 2.06E−09 | 8.75E−04 | −0.226428991 | 0.226428991 | loss | 0.872373709 | 0.645944719 | GLB1L2 | 37 | 11 |
| cg04059863 | 7.13E−10 | 3.03E−04 | −0.226424272 | 0.226424272 | loss | 0.886614419 | 0.660190147 | ILDR1; ILDR1; ILDR1 | 37 | 3 |
| cg01081083 | 9.23E−10 | 3.92E−04 | −0.226384747 | 0.226384747 | loss | 0.825574347 | 0.5991896 | SGSM1; SGSM1; SGSM1; SGSM1 | 37 | 22 |
| cg09737078 | 7.13E−10 | 3.03E−04 | −0.226382039 | 0.226382039 | loss | 0.754745704 | 0.528363665 | | 37 | 16 |
| cg19948393 | 1.01E−09 | 4.27E−04 | −0.226356312 | 0.226356312 | loss | 0.580058042 | 0.353701729 | ANKRD33; ANKRD33 | 37 | 12 |
| cg20616108 | 2.16E−09 | 9.17E−04 | −0.226327458 | 0.226327458 | loss | 0.528412917 | 0.302085459 | | 37 | 1 |
| cg17546623 | 1.10E−09 | 4.65E−04 | −0.226305854 | 0.226305854 | loss | 0.813323272 | 0.587017418 | | 37 | 10 |
| cg13048466 | 7.13E−10 | 3.03E−04 | −0.226287529 | 0.226287529 | loss | 0.57959007 | 0.353302541 | | 37 | 1 |
| cg24608087 | 1.01E−09 | 4.27E−04 | −0.226281916 | 0.226281916 | loss | 0.71313414 | 0.486852224 | | 37 | X |
| cg16138602 | 7.13E−10 | 3.03E−04 | −0.226256544 | 0.226256544 | loss | 0.728562232 | 0.520305688 | TSPAN11 | 37 | 12 |
| cg23925513 | 9.23E−10 | 3.92E−04 | −0.226255769 | 0.226255769 | loss | 0.677245845 | 0.450990076 | MIR497HG | 37 | 17 |
| cg20410290 | 2.78E−09 | 1.18E−03 | −0.226254547 | 0.226254547 | loss | 0.671996558 | 0.445742012 | | 37 | 17 |
| cg04562491 | 7.13E−10 | 3.03E−04 | −0.226245028 | 0.226245028 | loss | 0.53535244 | 0.309107412 | DTX1 | 37 | 12 |
| cg05072008 | 3.02E−09 | 1.28E−03 | −0.226238007 | 0.226238007 | loss | 0.429301119 | 0.203063112 | FIGNL1; FIGNL1 | 37 | 7 |
| cg14930320 | 7.13E−10 | 3.03E−04 | −0.226205685 | 0.226205685 | loss | 0.666553485 | 0.4403478 | | 37 | 6 |
| cg08667128 | 9.34E−10 | 3.97E−04 | −0.226199701 | 0.226199701 | loss | 0.336454065 | 0.110254365 | MIR193A | 37 | 17 |
| cg21901395 | 7.13E−10 | 3.03E−04 | −0.226183267 | 0.226183267 | loss | 0.591778702 | 0.365595435 | | 37 | 17 |
| cg02451774 | 4.57E−09 | 1.94E−03 | −0.226156751 | 0.226156751 | loss | 0.387236187 | 0.161079435 | NBPF8; NBPF8; NBPF8 | 37 | 1 |
| cg17016890 | 2.35E−09 | 9.98E−04 | −0.226137722 | 0.226137722 | loss | 0.982087651 | 0.755949929 | ZBTB7C | 37 | 18 |
| cg00082939 | 7.77E−10 | 3.30E−04 | −0.226108879 | 0.226108879 | loss | 0.528650885 | 0.302542006 | ZNFS03-A52; ZNS503-AS2 | 37 | 10 |
| cg13660719 | 7.13E−10 | 3.03E−04 | −0.226101841 | 0.226101841 | loss | 0.456889511 | 0.230787671 | DDR1; DDR1; DDR1 | 37 | 6 |
| cg18512313 | 3.02E−09 | 1.28E−03 | −0.226071277 | 0.226071277 | loss | 0.870287089 | 0.644215812 | | 37 | 14 |
| cg25383593 | 7.13E−10 | 3.03E−04 | −0.226041581 | 0.226041581 | loss | 0.33268154 | 0.106639959 | | 37 | 5 |
| cg13703912 | 6.35E−09 | 2.70E−03 | −0.226020912 | 0.226020912 | loss | 0.731959536 | 0.505938624 | C9orf66; DOCK8 | 37 | 9 |
| cg05422049 | 7.84E−10 | 3.33E−04 | −0.22600771 | 0.22600771 | loss | 0.361892823 | 0.135885114 | | 37 | 2 |
| cg27277859 | 7.13E−10 | 3.03E−04 | −0.226007324 | 0.226007324 | loss | 0.8018679 | 0.575860576 | SHANK1 | 37 | 19 |
| cg10134527 | 7.13E−10 | 3.03E−04 | −0.225994363 | 0.225994363 | loss | 0.879048098 | 0.653053735 | ZBTB22; TAPBP; ZBTB22; TAPBP; | 37 | 6 |
| | | | | | | | | TAPBP | | |
| cg21153102 | 7.48E−09 | 3.18E−03 | −0.225979584 | 0.225979584 | loss | 0.53272346 | 0.306743876 | | 37 | 15 |
| cg23272544 | 7.13E−10 | 3.03E−04 | −0.225920564 | 0.225920564 | loss | 0.936080611 | 0.710160047 | AIRE | 37 | 21 |
| cg11438310 | 1.30E−09 | 5.52E−04 | −0.22591857 | 0.22591857 | loss | 0.555551911 | 0.329633341 | CMYA5 | 37 | 5 |
| cg19187616 | 3.57E−09 | 1.52E−03 | −0.225901246 | 0.225901246 | loss | 0.577725028 | 0.351823782 | | 37 | 8 |
| cg12424468 | 7.13E−10 | 3.03E−04 | −0.22587994 | 0.22587994 | loss | 0.858684492 | 0.632804553 | VPS51; VPS51 | 37 | 11 |
| cg23998478 | 7.13E−10 | 3.03E−04 | −0.225870725 | 0.225870725 | loss | 0.358184883 | 0.132314158 | | 37 | 2 |
| cg11262216 | 8.47E−10 | 3.60E−04 | −0.225865765 | 0.225865765 | loss | 0.693172483 | 0.467306718 | MEOX1; MEOX1; MEOX1 | 37 | 17 |
| cg19608003 | 7.13E−10 | 3.03E−04 | −0.225837443 | 0.225837443 | loss | 0.842203802 | 0.616366359 | | 37 | 20 |
| cg14769991 | 7.13E−10 | 3.03E−04 | −0.225792458 | 0.225792458 | loss | 0.58115147 | 0.355359012 | | 37 | 6 |
| cg17700835 | 1.54E−09 | 6.54E−04 | −0.225766554 | 0.225766554 | loss | 0.72797226 | 0.502205706 | | 37 | 6 |
| cg21086940 | 5.16E−09 | 2.19E−03 | −0.225757607 | 0.225757607 | loss | 0.377950075 | 0.152192468 | | 37 | 8 |
| cg21962013 | 7.13E−10 | 3.03E−04 | −0.22575659 | 0.22575659 | loss | 0.630737249 | 0.404980659 | ATP8A2 | 37 | 13 |
| cg23769143 | 7.13E−10 | 3.03E−04 | −0.225743493 | 0.225743493 | loss | 0.56029334 | 0.334549847 | SYN2; TIMP4; TIMP4; SYN2 | 37 | 3 |
| cg23133355 | 7.13E−10 | 3.03E−04 | −0.225741348 | 0.225741348 | loss | 0.93010023 | 0.704358882 | | 37 | 7 |
| cg13613044 | 7.77E−10 | 3.30E−04 | −0.225716081 | 0.225716081 | loss | 0.846126428 | 0.620410347 | | 37 | 9 |
| cg20436037 | 7.13E−10 | 3.03E−04 | −0.225689095 | 0.225689095 | loss | 0.987159377 | 0.761470282 | FAM83C | 37 | 20 |
| cg26730347 | 3.97E−08 | 1.69E−02 | −0.225685495 | 0.225685495 | loss | 0.513438125 | 0.287752629 | SLC5A1 | 37 | 22 |
| cg09163005 | 7.13E−10 | 3.03E−04 | −0.225671232 | 0.225671232 | loss | 0.336076445 | 0.110405214 | H2AFJ; H2AFJ | 37 | 12 |
| cg02299328 | 8.47E−10 | 3.60E−04 | −0.22565211 | 0.22565211 | loss | 0.425985757 | 0.200333647 | RAPGEFL1 | 37 | 17 |
| cg01775897 | 7.13E−10 | 3.03E−04 | −0.225650934 | 0.225650934 | loss | 0.516568375 | 0.290917441 | DPF3; DPF3; DPF3; DPF3 | 37 | 14 |
| cg21626848 | 7.77E−10 | 3.30E−04 | −0.225631799 | 0.225631799 | loss | 0.462202981 | 0.236571182 | LEPREL4; FKBP10 | 37 | 17 |
| cg05375686 | 3.88E−09 | 1.65E−03 | −0.225618708 | 0.225618708 | loss | 0.721069302 | 0.495450594 | | 37 | 12 |
| cg07895437 | 1.19E−09 | 5.07E−04 | −0.225618105 | 0.225618105 | loss | 0.430848234 | 0.205230129 | ZBTB22; TAPBP; ZBTB22; TAPBP; | 37 | 6 |
| | | | | | | | | TAPBP | | |
| cg13320683 | 7.13E−10 | 3.03E−04 | −0.225627978 | 0.225617978 | loss | 0.710495272 | 0.484877294 | RHOBTB1; RHOBTB1 | 37 | 10 |
| cg23198707 | 1.01E−09 | 4.27E−04 | −0.225609408 | 0.225609408 | loss | 0.628870996 | 0.403261588 | | 37 | 3 |
| cg11022060 | 6.30E−08 | 2.68E−02 | −0.225599786 | 0.225599786 | loss | 0.671792568 | 0.446192782 | CPEB1 | 37 | 15 |
| cg06636220 | 2.35E−09 | 9.98E−04 | −0.225591172 | 0.225591172 | loss | 0.881596625 | 0.656005453 | | 37 | 3 |
| cg25323057 | 1.73E−09 | 7.34E−04 | −0.225588055 | 0.225588055 | loss | 0.332760323 | 0.107172268 | ADIRF; AGAP11 | 37 | 10 |
| cg18063278 | 6.89E−09 | 2.93E−03 | −0.225555391 | 0.225555391 | loss | 0.674946938 | 0.449391547 | PPARG; PPARG | 37 | 3 |
| cg14976741 | 1.42E−09 | 6.01E−04 | −0.225517031 | 0.225517031 | loss | 0.607791919 | 0.382274888 | GALNT8 | 37 | 12 |
| cg20400592 | 7.13E−10 | 3.03E−04 | −0.225509097 | 0.225509097 | loss | 0.760605015 | 0.535095918 | DLEU7; DLEU7; DLEU7- | 37 | 13 |
| | | | | | | | | AS1 | | |
| cg05339727 | 2.12E−08 | 9.01E−03 | −0.225484641 | 0.225484641 | loss | 0.483859158 | 0.258374518 | SLC38A11; SLC38A11; SLC38A11; | 37 | 2 |
| | | | | | | | | SLC38A11 | | |
| cg24937995 | 4.29E−08 | 1.82E−02 | −0.225461027 | 0.225461027 | loss | 0.907389315 | 0.681928288 | | 37 | 21 |
| cg04937609 | 7.13E−10 | 3.03E−04 | −0.225454098 | 0.225454098 | loss | 0.710771404 | 0.485317306 | KLHL29 | 37 | 2 |
| cg23411869 | 7.13E−10 | 3.03E−04 | −0.225421111 | 0.225421111 | loss | 0.861561117 | 0.636140006 | | 37 | 17 |
| cg00137234 | 1.01E−09 | 4.27E−04 | −0.225421048 | 0.225421048 | loss | 0.644688983 | 0.419267935 | RBM46; RBM46; RBM46 | 37 | 4 |
| cg18040225 | 7.13E−10 | 3.03E−04 | −0.225399232 | 0.225399232 | loss | 0.581353996 | 0.355954765 | CCNJ; CCNJ; CCNJ; ENTPD1- | 37 | 10 |
| | | | | | | | | AS1 | | |
| cg00147850 | 1.10E−09 | 4.65E−04 | −0.225397487 | 0.225397487 | loss | 0.877959523 | 0.652562035 | PDE4DIP; PDE4DIP; PDE4DIP; | 37 | 1 |
| | | | | | | | | PDE4DIP; PDE4DIP; PDE4DIP; | | |
| | | | | | | | | NBPF9; LOC100288142; | | |
| | | | | | | | | PDE4DIP; PDE4DIP | | |
| cg02710534 | 8.47E−10 | 3.60E−04 | −0.225389121 | 0.225389121 | loss | 0.918915304 | 0.693526182 | | 37 | 5 |
| cg10994149 | 1.68E−09 | 7.12E−04 | −0.225384879 | 0.225384879 | loss | 0.777421438 | 0.552036559 | | 37 | 5 |
| cg239S8524 | 7.13E−10 | 3.03E−04 | −0.225380609 | 0.225380609 | loss | 0.745599632 | 0.520219024 | SRGAP3; SRGAP3 | 37 | 3 |
| cg27217474 | 7.13E−10 | 3.03E−04 | −0.225357688 | 0.225357688 | loss | 0.824909464 | 0.599551776 | | 37 | 17 |
| cg24159514 | 4.75E−09 | 2.02E−03 | 0.225312 | 0.225312 | gain | 0.500469877 | 0.725781876 | ZNF833P | 37 | 19 |
| cg26681628 | 1.42E−09 | 6.01E−04 | −0.225304988 | 0.225304988 | loss | 0.683414553 | 0.453109565 | | 37 | 16 |
| cg10312186 | 7.13E−10 | 3.03E−04 | −0.225300902 | 0.225300902 | gain | 0.393735492 | 0.619036394 | | 37 | 6 |
| cg07208667 | 7.13E−10 | 3.03E−04 | −0.225300484 | 0.225300484 | loss | 0.66917656 | 0.443876076 | | 37 | 8 |
| cg00594129 | 7.13E−10 | 3.03E−04 | −0.225287516 | 0.225237516 | loss | 0.365764087 | 0.140476571 | EBF1 | 37 | 5 |
| cg23059946 | 8.47E−10 | 3.60E−04 | −0.225273311 | 0.225273311 | loss | 0.649380328 | 0.424107018 | IGFBP2 | 37 | 2 |
| cg27264390 | 7.13E−10 | 3.03E−04 | −0.22525221 | 0.22525221 | loss | 0.803192404 | 0.577940194 | | 37 | 8 |
| cg01345055 | 1.18E−08 | 5.01E−03 | −0.22524972 | 0.22524972 | loss | 0.554931296 | 0.329681576 | CPLX1 | 37 | 4 |
| cg14579240 | 7.13E−10 | 3.03E−04 | −0.225233946 | 0.225233946 | loss | 0.553623528 | 0.328389582 | EXD3 | 37 | 9 |
| cg26403223 | 3.02E−09 | 1.28E−03 | −0.225217095 | 0.225217095 | loss | 0.86193643 | 0.636719335 | | 37 | 1 |
| cg00489772 | 7.13E−10 | 3.03E−04 | −0.225193719 | 0.225193719 | loss | 0.791465913 | 0.566272194 | DFFB; DFFB; CEP104; DFFB | 37 | 1 |
| cg16968985 | 7.13E−10 | 3.03E−04 | −0.225169931 | 0.225169931 | loss | 0.424649313 | 0.199479382 | SEZ6; SEZ6 | 37 | 17 |
| cg03776087 | 6.89E−09 | 2.93E−03 | −0.225152378 | 0.225152378 | loss | 0.802337255 | 0.577184876 | TRPM8 | 37 | 2 |
| cg05673731 | 7.13E−10 | 3.03E−04 | −0.225133755 | 0.225133755 | loss | 0.438436985 | 0.213303229 | PTPRU; PTPRU; PTPRU; PTPRU | 37 | 1 |
| cg23873996 | 1.57E−09 | 6.68E−04 | −0.225131385 | 0.225131385 | loss | 0.53544355 | 0.310312165 | | 37 | 6 |
| cg12889810 | 4.57E−09 | 1.94E−03 | −0.225114201 | 0.225114201 | loss | 0.830755877 | 0.605641676 | | 37 | 7 |
| cg03240511 | 9.23E−10 | 3.92E−04 | −0.225105906 | 0.225105906 | loss | 0.342407289 | 0.117301383 | | 37 | 16 |
| cg15444185 | 7.13E−10 | 3.03E−04 | −0.225083897 | 0.225083897 | loss | 0.413680685 | 0.188596788 | | 37 | 8 |
| cg10099601 | 8.47E−10 | 3.60E−04 | −0.225081549 | 0.225081549 | loss | 0.659961496 | 0.434879947 | TRIL | 37 | 7 |
| cg21645554 | 7.13E−10 | 3.03E−04 | −0.225057068 | 0.225057068 | loss | 0.374410294 | 0.149353226 | COLCA2; COLCA2; COLCA2; | 37 | 11 |
| | | | | | | | | COLCA1 | | |
| cg09124496 | 8.80E−09 | 3.74E−03 | −0.225053777 | 0.225053777 | loss | 0.829401483 | 0.604347706 | INHBA; INHBA-AS1; INHBA- | 37 | 7 |
| | | | | | | | | AS1 | | |
| cg06399596 | 1.32E−08 | 5.59E−03 | −0.225047372 | 0.225047372 | loss | 0.670047602 | 0.445000229 | | 37 | 6 |
| cg14887142 | 8.47E−10 | 3.60E−04 | −0.225042094 | 0.225042094 | loss | 0.591157847 | 0.366115753 | | 37 | 14 |
| cg11427534 | 8.47E−10 | 3.60E−04 | −0.225037746 | 0.225037746 | loss | 0.429840281 | 0.204802535 | | 37 | 5 |
| cg04763519 | 7.13E−10 | 3.03E−04 | −0.225023844 | 0.225023844 | loss | 0.734189079 | 0.509165235 | | 37 | 6 |
| cg27329371 | 1.42E−09 | 6.01E−04 | −0.225005354 | 0.225005354 | loss | 0.758367566 | 0.533362212 | ALDH3A1; ALDH3A1 | 37 | 17 |
| cg24406898 | 2.69E−08 | 1.14E−02 | −0.224975217 | 0.224975217 | loss | 0.729332864 | 0.504357647 | COL1A2 | 37 | 7 |
| cg17248924 | 8.55E−08 | 3.63E−02 | −0.224972103 | 0.224972103 | loss | 0.391299558 | 0.166327456 | TMEM232; TMEM232 | 37 | 5 |
| cg11245681 | 1.42E−09 | 6.01E−04 | −0.224937415 | 0.224937415 | loss | 0.514429457 | 0.289492041 | | 37 | 5 |
| cg16207110 | 8.47E−10 | 3.60E−04 | −0.22493733 | 0.22493733 | loss | 0.318696551 | 0.093759221 | | 37 | 17 |
| cg01601949 | 1.19E−09 | 5.07E−04 | −0.224928719 | 0.224928719 | loss | 0.540790555 | 0.315861835 | | 37 | 2 |
| cg14253670 | 1.42E−09 | 6.01E−04 | −0.224909582 | 0.224909582 | loss | 0.773443547 | 0.548533965 | | 37 | 8 |
| cg03390419 | 7.13E−10 | 3.03E−04 | −0.224897477 | 0.224897477 | loss | 0.580094247 | 0.355196771 | DUPD1 | 37 | 10 |
| cg01497892 | 1.43E−08 | 6.06E−03 | −0.224891499 | 0.224891409 | loss | 0.736457128 | 0.511565629 | | 37 | 6 |
| cg11137156 | 7.13E−10 | 3.03E−04 | −0.224886578 | 0.224886578 | loss | 0.837059913 | 0.612173335 | | 37 | 3 |
| cg01827781 | 7.13E−10 | 3.03E−04 | −0.224872874 | 0.224872874 | loss | 0.671826751 | 0.446953876 | CDIPT; CDIPT; CDIPT; CDIPT- | 37 | 16 |
| | | | | | | | | AS1; CDIPT-AS1 | | |
| cg24790297 | 1.30E−09 | 5.52E−04 | −0.224846301 | 0.224846301 | loss | 0.493555772 | 0.268709471 | CLEC11A | 37 | 19 |
| cg03862380 | 7.77E−10 | 3.30E−04 | −0.224842512 | 0.224842512 | loss | 0.725541142 | 0.500698629 | HNF4A; HNF4A; HNF4A; HNF4A; | 37 | 20 |
| | | | | | | | | HNF4A; HNF4A; HNF4A | | |
| cg20256260 | 7.13E−10 | 3.03E−04 | −0.224837036 | 0.224837036 | loss | 0.361382591 | 0.136545555 | RBPJL; RBPJL; MATN4; MATN4; | 37 | 20 |
| | | | | | | | | RBPJL; MATN4; MATN4; | | |
| | | | | | | | | MATN4; MATN4 | | |
| cg16681268 | 1.19E−09 | 5.07E−04 | −0.224832761 | 0.224832761 | loss | 0.926764732 | 0.701931971 | ASZ1; ASZ1; ASZ1; ASZ1 | 37 | 7 |
| cg22160472 | 7.84E−10 | 3.33E−04 | −0.22482793 | 0.22482793 | loss | 0.460807365 | 0.235979435 | | 37 | 5 |
| cg08792388 | 1.42E−09 | 6.01E−04 | −0.224822788 | 0.224822788 | loss | 0.853765953 | 0.628943165 | | 37 | 6 |
| cg09629631 | 2.16E−09 | 9.17E−04 | −0.224817692 | 0.224817692 | loss | 0.754150992 | 0.5293333 | CACNA2D4 | 37 | 12 |
| cg05550995 | 8.47E−10 | 3.60E−04 | −0.22481011 | 0.22481011 | loss | 0.806970587 | 0.582160476 | | 37 | 16 |
| cg06241901 | 7.13E−10 | 3.03E−04 | −0.224809056 | 0.224809056 | loss | 0.514832168 | 0.290023112 | TTBK1 | 37 | 6 |
| cg15234492 | 8.56E−10 | 3.63E−04 | −0.224775416 | 0.224775416 | loss | 0.407043463 | 0.182268047 | KDM28; KDM2B; KDM2B | 37 | 12 |
| cg13526264 | 1.22E−08 | 5.16E−03 | −0.224764756 | 0.224764756 | loss | 0.380326642 | 0.155561886 | | 37 | 6 |
| cg19508900 | 7.13E−10 | 3.03E−04 | −0.224746597 | 0.224746597 | loss | 0.358887549 | 0.134140952 | ZBTB48; ZBTB48; ZBTB48; | 37 | 1 |
| | | | | | | | | TAS1R1; TAS1R1 | | |
| cg02026498 | 8.47E−10 | 3.60E−04 | −0.224746373 | 0.224746373 | loss | 0.459419091 | 0.234672718 | GNG8 | 37 | 19 |
| cg09497409 | 1.32E−08 | 5.59E−03 | −0.224745848 | 0.224745848 | loss | 0.471528715 | 0.246782867 | CER54 | 37 | 19 |
| cg10331119 | 7.13E−10 | 3.03E−04 | −0.224708049 | 0.224708049 | loss | 0.902003143 | 0.677295094 | CAMTA1 | 37 | 1 |
| cg01271455 | 1.42E−09 | 6.01E−04 | −0.22469551 | 0.22469551 | loss | 0.413073787 | 0.188378276 | PTPRN2; PTPRN2; PTPRN2 | 37 | 7 |
| cg01688776 | 7.13E−10 | 3.03E−04 | −0.224672889 | 0.224672889 | loss | 0.881499619 | 0.656826729 | LINC00112; LINC00479; LINC00479 | 37 | 21 |
| cg06975979 | 1.99E−09 | 8.43E−04 | −0.224665696 | 0.224665696 | loss | 0.342095349 | 0.117429654 | LRRC61; ACTR3C; LRRC61 | 37 | 7 |
| cg06687640 | 7.13E−10 | 3.03E−04 | −0.224644719 | 0.224644719 | loss | 0.301095364 | 0.076450645 | | 37 | 5 |
| cg12054981 | 1.54E−08 | 6.56E−03 | −0.22464249 | 0.22464249 | loss | 0.392217038 | 0.167574548 | PLAT; PLAT | 37 | 8 |
| cg12491114 | 7.13E−10 | 3.03E−04 | −0.224634131 | 0.224634131 | loss | 0.435403708 | 0.210769576 | KRT19 | 37 | 17 |
| cg14462779 | 7.13E−10 | 3.03E−04 | −0.224630852 | 0.224630852 | loss | 0.385292781 | 0.160661929 | DUPD1 | 37 | 10 |
| cg15730491 | 3.14E−08 | 1.33E−02 | −0.22462938 | 0.22462938 | loss | 0.693243615 | 0.468614235 | | 37 | 1 |
| cg12302402 | 7.13E−10 | 3.03E−04 | −0.224628303 | 0.224628303 | loss | 0.428460679 | 0.203832376 | LOC494141; LOC494141; LOC494141 | 37 | 11 |
| cg04512892 | 8.11E−09 | 3.44E−03 | −0.224618786 | 0.224618786 | loss | 0.426963013 | 0.202344228 | | 37 | 19 |
| cg00777271 | 1.42E−09 | 6.01E−04 | −0.224612788 | 0.224612788 | loss | 0.752521 | 0.527908212 | MTMR11; MTMR11 | 37 | 1 |
| cg10833838 | 4.57E−09 | 1.94E−03 | −0.224582263 | 0.224582263 | loss | 0.777868581 | 0.553286318 | MIR54BW | 37 | 17 |
| cg14731570 | 9.23E−10 | 3.92E−04 | −0.224578866 | 0.224578866 | loss | 0.51761786 | 0.293038994 | | 37 | 2 |
| cg03127898 | 7.13E−10 | 3.03E−04 | −0.224575834 | 0.224575834 | loss | 0.612653092 | 0.388077259 | LRRN4CL | 37 | 11 |
| cg05800368 | 1.54E−08 | 6.5GE−03 | −0.224542603 | 0.224542603 | loss | 0.681120932 | 0.456578329 | TTLL11 | 37 | 9 |
| cg24032393 | 8.47E−10 | 3.60E−04 | −0.224833869 | 0.224533869 | loss | 0.76863914 | 0.544105271 | NOS3 | 37 | 7 |
| cg25026992 | 7.34E−08 | 3.12E−02 | −0.224470648 | 0.224470648 | gain | 0.430817717 | 0.655288365 | PCDHB7 | 37 | 5 |
| cg11785154 | 2.35E−09 | 9.98E−04 | −0.224468206 | 0.224468206 | loss | 0.725902358 | 0.501434153 | SYT2 | 37 | 1 |
| cg13523576 | 7.13E−10 | 3.03E−04 | −0.224467399 | 0.224467399 | loss | 0.671046523 | 0.446579124 | DMTN | 37 | 8 |
| cg00056066 | 8.80E−09 | 3.74E−03 | −0.224451782 | 0.224451782 | loss | 0.704211558 | 0.479759776 | ARHGAP21 | 37 | 10 |
| cg17092956 | 7.13E−10 | 3.03E−04 | −0.224405978 | 0.224405978 | loss | 0.398860355 | 0.174454376 | | 37 | 10 |
| cg19846609 | 8.60E−09 | 3.65E−03 | −0.22440553 | 0.22440553 | loss | 0.960907724 | 0.736502194 | | 37 | 11 |
| cg17485681 | 1.30E−09 | 5.52E−04 | −0.224392919 | 0.224392919 | loss | 0.636350766 | 0.411957847 | CDH23; CDH23; CDK23 | 37 | 10 |
| cg26819718 | 1.73E−09 | 7.34E−04 | −0.224390266 | 0.224390266 | loss | 0.943532966 | 0.7191427 | C10orf11 | 37 | 10 |
| cg13794863 | 7.77E−10 | 330E−04 | −0.224387598 | 0.224387598 | loss | 0.826752498 | 0.6023649 | | 37 | 1 |
| cg06137072 | 7.84E−10 | 3.33E−04 | −0.224347133 | 0.224347133 | loss | 0.766254104 | 0.541906971 | ZBTB38 | 37 | 3 |
| cg15236483 | 4.64E−08 | 1.97E−02 | −0.224340792 | 0.224340792 | loss | 0.356015525 | 0.131674732 | | 37 | 22 |
| cg19252931 | 7.13E−10 | 3.03E−04 | −0.224318953 | 0.224318953 | loss | 0.355217381 | 0.130898428 | C11orf91 | 37 | 11 |
| cg04451175 | 7.13E−10 | 3.03E−04 | −0.224311039 | 0.224311039 | loss | 0.578697057 | 0.354386018 | FGF20 | 37 | 8 |
| cg11234688 | 1.03E−08 | 4.39E−03 | −0.224308416 | 0.224308416 | loss | 0.527154404 | 0.302845988 | | 37 | 2 |
| cg00341060 | 7.13E−10 | 3.03E−04 | −0.224307504 | 0.224307504 | loss | 0.742364345 | 0.518056841 | | 37 | 1 |
| cg15249793 | 3.88E−09 | 1.65E−03 | −0.224306223 | 0.224306223 | loss | 0.40714497 | 0.182838747 | TEX14; TEX14; TEX14 | 37 | 17 |
| cg18454045 | 7.13E−10 | 3.03E−04 | −0.224291515 | 0.224291515 | loss | 0.672243721 | 0.447952206 | | 37 | 10 |
| cg13812393 | 2.12E−08 | 9.01E−03 | −0.224277498 | 0.224277498 | loss | 0.648099009 | 0.423821512 | | 37 | 2 |
| cg20243176 | 7.13E−10 | 3.03E−04 | −0.224246739 | 0.224246739 | loss | 0.849342415 | 0.625095676 | | 37 | 1 |
| cg17191109 | 9.23E−10 | 3.92E−04 | −0.224196979 | 0.224196979 | loss | 0.821898426 | 0.597701447 | | 37 | 10 |
| cg2180B635 | 1.83E−09 | 7.75E−04 | −0.224188249 | 0.224188249 | loss | 0.811946743 | 0.587758494 | EGFR; EGFR; EGFR | 37 | 7 |
| cg10599948 | 7.13E−10 | 3.03E−04 | −0.224179278 | 0.224179278 | loss | 0.491942449 | 0.267763171 | RAS1P1; IZUMO1 | 37 | 19 |
| cg01079738 | 7.13E−10 | 3.03E−04 | −0.224179172 | 0.224179172 | loss | 0.851096225 | 0.626917053 | SHCBP1L; SHCBP1L | 37 | 1 |
| cg12902206 | 1.21E−09 | 5.15E−04 | −0.224157073 | 0.224157073 | loss | 0.452327962 | 0.228170888 | DNAH1 | 37 | 3 |
| cg00330953 | 3.14E−08 | 1.33E−02 | −0.224152541 | 0.224152541 | loss | 0.776890864 | 0.552738324 | VWA7 | 37 | 6 |
| cg09614876 | 4.97E−09 | 2.11E−03 | −0.224127112 | 0.224127112 | loss | 0.755263783 | 0.531136671 | LRRC66 | 37 | 4 |
| cg21918786 | 1.89E−09 | 8.02E−04 | −0.224119531 | 0.224119531 | loss | 0.9426295 | 0.718509969 | | 37 | 6 |
| cg10173186 | 7.13E−10 | 3.03E−04 | −0.223987246 | 0.223987246 | loss | 0.316589804 | 0.092602558 | SYN2; TIMP4; SYN2 | 37 | 3 |
| cg14830045 | 1.42E−09 | 6.01E−04 | −0.2239743 | 0.2239743 | loss | 0.726395442 | 0.502421141 | | 37 | 1 |
| cg13871843 | 9.54E−09 | 4.05E−03 | −0.223960858 | 0.223960858 | loss | 0.84903817 | 0.628077312 | | 37 | 1 |
| cg14629055 | 7.13E−10 | 3.03E−04 | −0.22394908 | 0.22394908 | loss | 0.789503551 | 0.565554471 | ANKRD338 | 37 | 5 |
| cg17658717 | 5.39E−09 | 2.29E−03 | −0.223917629 | 0.223917629 | loss | 0.521072217 | 0.297154588 | CLEC3B | 37 | 3 |
| cg15484406 | 1.30E−09 | 5.52E−04 | −0.223906569 | 0.223906569 | loss | 0.685089934 | 0.461183365 | | 37 | 14 |
| Cg22803560 | 3.57E−09 | 1.52E−03 | −0.223865352 | 0.223865352 | loss | 0.428457511 | 0.204592159 | | 37 | 4 |
| cg17127823 | 7.13E−10 | 3.03E−04 | −0.2238648 | 0.2238648 | loss | 0.402839053 | 0.178974253 | TRAM1; LOC286190 | 37 | 8 |
| cg09971646 | 2.91E−08 | 1.23E−02 | −0.22380044 | 0.22380044 | loss | 0.510506228 | 0.286705788 | DLK1 | 37 | 14 |
| cg21697794 | 7.77E−10 | 3.30E−04 | −0.223783385 | 0.223783385 | loss | 0.521350443 | 0.297567059 | | 37 | 6 |
| cg00366917 | 1.54E−09 | 6.54E−04 | −0.223782839 | 0.223782839 | loss | 0.486536904 | 0.262754065 | MBP; M3P | 37 | 18 |
| cg10203922 | 8.11E−09 | 3.44E−03 | −0.223775372 | 0.223775372 | loss | 0.717520002 | 0.493744629 | HHIP; HHIP-AS1 | 37 | 4 |
| cg23690302 | 4.97E−09 | 2.11E−03 | −0.223758818 | 0.223758818 | loss | 0.903008789 | 0.679249971 | | 37 | 4 |
| cg20371266 | 7.13E−10 | 3.03E−04 | −0.223755252 | 0.223755252 | loss | 0.304398317 | 0.080643065 | CHRND; CHRND; CHRND; CHRND | 37 | 2 |
| cg05082738 | 7.13E−10 | 3.03E−04 | −0.223752036 | 0.223752036 | loss | 0.858908436 | 0.6351564 | LINC00301 | 37 | 11 |
| cg22981993 | 7.13E−10 | 3.03E−04 | −0.223741394 | 0.223741394 | loss | 0.431439664 | 0.207698271 | ALS2CR11; ALS2CR11; ALS2CR11; | 37 | 2 |
| | | | | | | | | ALS2CR11; ALS2CR11; | | |
| | | | | | | | | ALS2CR11; ALS2CR11; | | |
| | | | | | | | | ALS2CR11 | | |
| cg09623056 | 7.13E−10 | 3.03E−04 | −0.223738942 | 0.223738942 | loss | 0.749959483 | 0.526220541 | NEURL3 | 37 | 2 |
| cg06847624 | 9.54E−09 | 4.05E−03 | −0.223717473 | 0.223717473 | loss | 0.318593687 | 0.094876214 | PFN3; PFN3 | 37 | 5 |
| cg27050936 | 6.89E−09 | 2.93E−03 | −0.223714603 | 0.223714603 | loss | 0.695108062 | 0.471393459 | | 37 | 6 |
| cg01969701 | 7.13E−10 | 3.03E−04 | −0.223693462 | 0.223693462 | loss | 0.254747992 | 0.0310S453 | | 37 | 11 |
| cg10237252 | 7.13E−10 | 3.03E−04 | −0.223684614 | 0.223684614 | loss | 0.553657632 | 0.329973018 | TOM1L1 | 37 | 17 |
| cg18232772 | 8.47E−10 | 3.60E−04 | −0.22366207 | 0.22366207 | loss | 0.820774906 | 0.597112835 | | 37 | 22 |
| cg20110349 | 7.48E−09 | 3.18E−03 | −0.223641656 | 0.223641656 | loss | 0.465218921 | 0.241577265 | TO82P1 | 37 | 6 |
| cg26493188 | 8.80E−09 | 3.74E−03 | −0.223626883 | 0.223626883 | loss | 0.775825036 | 0.552198153 | FLYWCH1; FLYWCH1 | 37 | 16 |
| cg03387723 | 7.77E−10 | 3.30E−04 | −0.223613158 | 0.223613158 | loss | 0.657495664 | 0.433882506 | SCMH1; SCMH1; SCMH1; SCMH1 | 37 | 1 |
| cg10199168 | 8.47E−10 | 3.60E−04 | −0.223611756 | 0.223611756 | loss | 0.847807662 | 0.624195906 | DPEP3; DPEP3; DPEP3; DPEP3 | 37 | 16 |
| cg12050175 | 1.12E−08 | 4.76E−03 | −0.223611609 | 0.223611609 | loss | 0.675520362 | 0.451908753 | | 37 | 10 |
| cg15332871 | 8.47E−10 | 3.60E−04 | −0.223611006 | 0.223611006 | loss | 0.747085 | 0.523473994 | | 37 | 6 |
| cg25315503 | 7.13E−10 | 3.03E−04 | −0.223601091 | 0.223601091 | loss | 0.809280821 | 0.585679729 | | 37 | 21 |
| cg10702227 | 1.54E−09 | 6.54E−04 | −0.223600803 | 0.223600803 | loss | 0.464310126 | 0.240709324 | | 37 | 8 |
| cg21217093 | 3.97E−08 | 1.69E−02 | −0.223596366 | 0.223596366 | loss | 0.579925019 | 0.356328653 | | 37 | 9 |
| cg20370281 | 7.848-10 | 3.33E−04 | −0.223574089 | 0.223574089 | loss | 0.922814537 | 0.699240447 | VPS18 | 37 | 15 |
| cg08901520 | 5.85E−09 | 2.49E−03 | −0.223559058 | 0.223559058 | loss | 0.874913411 | 0.651354353 | | 37 | 2 |
| cg27149931 | 7.13E−10 | 3.03E−04 | −0.223519647 | 0.223519647 | loss | 0.429790906 | 0.206271259 | | 37 | 19 |
| cg00425710 | 7.13E−10 | 3.03E−04 | −0.223456977 | 0.223456977 | loss | 0.693559077 | 0.4701021 | MT1A | 37 | 16 |
| cg02369542 | 3.02E−09 | 1.28E−03 | −0.2234473 | 0.2234473 | loss | 0.5611127 | 0.3376654 | RIMS3 | 37 | 1 |
| cg09391284 | 7.13E−10 | 3.03E−04 | −0.223446105 | 0.223446105 | loss | 0.589871411 | 0.366425306 | | 37 | 3 |
| cg05644480 | 7.13E−10 | 3.03E−04 | −0.223428191 | 0.223428191 | loss | 0.524589891 | 0.3011617 | APOC1 | 37 | 19 |
| cg17936051 | 1.19E−09 | 5.07E−04 | −0.223410453 | 0.223410453 | loss | 0.799826189 | 0.576415735 | LINC00469; LOC400620 | 37 | 17 |
| cg21855021 | 4.97E−09 | 2.11E−03 | −0.223407049 | 0.223407049 | loss | 0.503083943 | 0.279676894 | AKAP7 | 37 | 6 |
| cg24389359 | 3.02E−09 | 1.28E−03 | −0.223406401 | 0.223406401 | loss | 0.781002713 | 0.557596312 | CCDC63; CCDC63; CCDC63 | 37 | 12 |
| cg13205937 | 1.10E−09 | 4.65E−04 | −0.22337339 | 0.22337339 | loss | 0.847110413 | 0.623737024 | DAGLA | 37 | 11 |
| cg01431340 | 1.03E−08 | 4.39E−03 | −0.223350815 | 0.223350815 | loss | 0.398683828 | 0.175333013 | METTL24 | 37 | 6 |
| cg17716663 | 1.10E−09 | 4.65E−04 | −0.223330229 | 0.223330229 | loss | 0.361014904 | 0.137684675 | FOSB; FOSB | 37 | 19 |
| cg03553576 | 7.13E−10 | 3.03E−04 | −0.223330009 | 0.223330009 | loss | 0.618999079 | 0.395669071 | LRRC14B | 37 | 5 |
| cg07142010 | 7.13E−10 | 3.03E−04 | −0.223269551 | 0.223269551 | loss | 0.335851087 | 0.112581536 | ALPL; ALPL; ALPL | 37 | 1 |
| cg20075683 | 1.10E−09 | 4.65E−04 | −0.223269523 | 0.223269523 | loss | 0.776950675 | 0.553681153 | CCNI2 | 37 | 5 |
| cg05131382 | 7.13E−10 | 3.03E−04 | −0.223263868 | 0.223263868 | loss | 0.612273857 | 0.389009988 | | 37 | 3 |
| cg23686113 | 1.01E−09 | 4.27E−04 | −0.223260837 | 0.223260837 | loss | 0.367620142 | 0.144359304 | STAU2; STAU2; STAU2; STAU2; | 37 | 8 |
| | | | | | | | | STAU2; STAU2; STAU2 | | |
| cg08121925 | 9.23E−10 | 3.92E−04 | −0.223257402 | 0.223257402 | loss | 0.366534536 | 0.143277134 | | 37 | 3 |
| cg25920019 | 6.80E−08 | 2.89E−02 | −0.223254456 | 0.223254456 | loss | 0.810046632 | 0.586792176 | | 37 | 5 |
| cg22880770 | 1.42E−09 | 6.01E−04 | −0.223218741 | 0.223218741 | loss | 0.551752658 | 0.328533918 | NEURL3; NEURL3; NEURL3 | 37 | 2 |
| cg21809624 | 7.13E−10 | 3.03E−04 | −0.223212134 | 0.223212134 | loss | 0.591095587 | 0.367883453 | | 37 | 17 |
| cg16405308 | 4.21E−09 | 1.79E−03 | −0.223210335 | 0.223210335 | loss | 0.840470411 | 0.617260076 | | 37 | 15 |
| cg17329202 | 7.13E−10 | 3.03E−04 | −0.223205635 | 0.223205635 | loss | 0.463604711 | 0.240399076 | | 37 | 13 |
| cg13871826 | 7.13E−10 | 3.03E−04 | −0.223161266 | 0.223161266 | loss | 0.773682125 | 0.550520859 | DDAH1; CYR61 | 37 | 1 |
| cg19414980 | 1.43E−08 | 6.06E−03 | −0.223142344 | 0.223142344 | loss | 0.680626126 | 0.457483782 | | 37 | 7 |
| cg00760646 | 7.13E−10 | 3.03E−04 | −0.223133711 | 0.223133711 | loss | 0.812659423 | 0.589525712 | CXCL16; ZMYND15; ZMYND15; | 37 | 17 |
| | | | | | | | | CXCL16; ZMYND15 | | |
| cg27056132 | 7.13E−10 | 3.03E−04 | −0.223102432 | 0.223102432 | loss | 0.601912879 | 0.378810447 | | 37 | 1 |
| cg16060189 | 7.77E−10 | 3.30E−04 | −0.223093694 | 0.223093694 | loss | 0.634657264 | 0.411563571 | | 37 | 10 |
| cg22463915 | 7.13E−10 | 3.03E−04 | −0.223048503 | 0.223048503 | loss | 0.574092185 | 0.351043682 | EEF1A2 | 37 | 20 |
| cg21412355 | 2.56E−09 | 1.09E−03 | −0.223039906 | 0.223039906 | loss | 0.741541953 | 0.518502047 | | 37 | 18 |
| cg19562969 | 7.13E−10 | 3.03E−04 | −0.223030885 | 0.223030885 | loss | 0.388329274 | 0.165298388 | C20orf24; TGIF2- | 37 | 20 |
| | | | | | | | | C20orf24; C20orf24; C20orf | | |
| | | | | | | | | 24; C20orf24 | | |
| cg02382400 | 7.13E−10 | 3.03E−04 | −0.223007804 | 0.223007804 | loss | 0.728549592 | 0.505541788 | SLC4A11; SLC4A11; SLC4A11; | 37 | 20 |
| | | | | | | | | SLC4A11 | | |
| cg07690882 | 4.57E−09 | 1.94E−03 | −0.223003043 | 0.223003043 | loss | 0.373881726 | 0.150878683 | | 37 | 11 |
| cg14276557 | 1.11E−09 | 4.72E−04 | −0.222978723 | 0.222978723 | loss | 0.51764654 | 0.294667818 | | 37 | 9 |
| cg06698332 | 1.12E−08 | 4.76E−03 | −0.222951272 | 0.222951272 | loss | 0.897334172 | 0.6743829 | S100A10 | 37 | 1 |
| cg20664247 | 7.77E−10 | 3.30E−04 | −0.222948221 | 0.222948221 | loss | 0.753478704 | 0.530530482 | PEX10; PEX10 | 37 | 1 |
| cg13213799 | 9.23E−10 | 3.92E−04 | −0.222947943 | 0.222947943 | loss | 0.802688196 | 0.579740253 | | 37 | 14 |
| cg17241776 | 7.13E−10 | 3.03E−04 | −0.222877869 | 0.222377869 | loss | 0.254131515 | 0.031253645 | DLEU7; DLEU7-AS1 | 37 | 13 |
| cg01540595 | 9.23E−10 | 3.92E−04 | −0.222870719 | 0.222870719 | loss | 0.530316666 | 0.307445947 | | 37 | 1 |
| cg09957942 | 7.77E−10 | 3.30E−04 | −0.222850951 | 0.222850951 | loss | 0.458200792 | 0.235349841 | | 37 | 13 |
| cg20119532 | 1.19E−09 | 5.07E−04 | −0.222846466 | 0.222846466 | loss | 0.655027155 | 0.432180688 | SLC52A3 | 37 | 20 |
| cg13978777 | 7.13E−10 | 3.03E−04 | −0.222842005 | 0.222842006 | loss | 0.670728942 | 0.447886935 | | 37 | 19 |
| cg17986341 | 7.13E−10 | 3.03E−04 | −0.222838715 | 0.222838715 | loss | 0.721837326 | 0.498998612 | TP73; TP73; TP73; TP73; TP73; | 37 | 1 |
| | | | | | | | | TP73; TP73; TP73; TP73; | | |
| | | | | | | | | TP73; TP73; TP73; TP73 | | |
| cg08286854 | 1.10E−09 | 4.65E−04 | −0.222807984 | 0.222807984 | loss | 0.587957696 | 0.365149712 | KRTAP2-2 | 37 | 17 |
| cg00531789 | 9.23E−10 | 3.92E−04 | −0.222803923 | 0.222803923 | loss | 0.851341123 | 0.6285372 | PI16; PI16 | 37 | 6 |
| cg02464921 | 7.13E−10 | 3.03E−04 | −0.222803353 | 0.222803353 | loss | 0.699825342 | 0.477021988 | | 37 | 10 |
| cg19498228 | 8.47E−10 | 3.60E−04 | −0.22278506 | 0.22278506 | loss | 0.603526625 | 0.380741565 | NPHS1; NPHS1 | 37 | 19 |
| cg17674604 | 1.30E−09 | 5.52E−04 | −0.222742533 | 0.222742533 | loss | 0.717631915 | 0.494889382 | PHYHIP; PHYHIP | 37 | 8 |
| cg15571882 | 1.68E−09 | 7.12E−04 | −0.222726063 | 0.222726063 | loss | 0.831547398 | 0.608821335 | SLC44A3; SLC44A3; SLC44A3; | 37 | 1 |
| | | | | | | | | SLC44A3; SLC44A3; SLC44A3; | | |
| | | | | | | | | LINC01057 | | |
| cg13388466 | 7.77E−10 | 3.30E−04 | −0.222692553 | 0.222692553 | loss | 0.592842906 | 0.370150353 | | 37 | 17 |
| cg17076485 | 7.13E−10 | 3.03E−04 | −0.222650708 | 0.222650708 | loss | 0.598601749 | 0.375951041 | | 37 | 11 |
| cg21386647 | 7.13E−10 | 3.03E−04 | −0.2226264 | 0.2226264 | loss | 0.698569611 | 0.475943212 | SRGAP3; SRGAP3 | 37 | 3 |
| cg03954086 | 3.02E−09 | 1.28E−03 | −0.222613868 | 0.222613868 | loss | 0.755324574 | 0.532710706 | | 37 | 12 |
| cg10663316 | 1.01E−09 | 4.27E−04 | −0.222609806 | 0.222609806 | loss | 0.7234728 | 0.500862994 | ZNF664-FAM101A | 37 | 12 |
| cg07691146 | 1.54E−09 | 6.54E−04 | −0.222591037 | 0.222591037 | loss | 0.821782049 | 0.599191012 | FAM124A; FAM124A | 37 | 13 |
| cg00499707 | 7.13E−10 | 3.03E−04 | −0.222575542 | 0.222575542 | loss | 0.811654236 | 0.589078694 | EPB41L1; EPB41L1; EPB41L1; | 37 | 20 |
| | | | | | | | | EPB41L1 | | |
| cg22258315 | 9.23E−10 | 3.92E−04 | −0.222570269 | 0.222570269 | loss | 0.493957051 | 0.271386782 | | 37 | 8 |
| cg15436123 | 2.78E−09 | 1.18E−03 | −0.222569469 | 0.222569469 | loss | 0.555202092 | 0.332632624 | RGS12; RGS12; RGS12 | 37 | 4 |
| cg03980370 | 7.13E−10 | 3.03E−04 | −0.222560788 | 0.222560788 | loss | 0.823628358 | 0.601067571 | ECT2L; ECT2L | 37 | 6 |
| cg25629041 | 7.13E−10 | 3.03E−04 | −0.222536345 | 0.222536345 | loss | 0.746845045 | 0.5243087 | | 37 | 2 |
| cg17696006 | 1.01E−09 | 4.27E−04 | −0.222497317 | 0.222497317 | loss | 0.503759975 | 0.281262659 | | 37 | 2 |
| cg15541583 | 1.42E−09 | 6.01E−04 | −0.222494134 | 0.222494134 | loss | 0.480731481 | 0.258237347 | SLC9C1; SLC9C1 | 37 | 3 |
| cg01359962 | 1.83E−09 | 7.75E−04 | −0.222462681 | 0.222462681 | loss | 0.551821728 | 0.329359047 | POMGNT2 | 37 | 3 |
| cg00697095 | 9.23E−10 | 3.92E−04 | −0.222436589 | 0.222436589 | loss | 0.835907836 | 0.613471247 | GRB7; GRB7; GRB7; GRB7 | 37 | 17 |
| cg08133641 | 7.13E−10 | 3.03E−04 | −0.222415692 | 0.222415692 | loss | 0.439813298 | 0.217397606 | TMEM190 | 37 | 19 |
| cg05308504 | 7.13E−10 | 3.03E−04 | −0.222413826 | 0.222413826 | loss | 0.242196434 | 0.019782607 | | 37 | 16 |
| cg01745789 | 7.77E−10 | 3.30E−04 | −0.222411233 | 0.222411233 | loss | 0.315219891 | 0.092808658 | | 37 | 7 |
| cg04707519 | 7.13E−10 | 3.03E−04 | −0.222369235 | 0.222369235 | loss | 0.239810779 | 0.017441544 | | 37 | 10 |
| cg15033653 | 7.13E−10 | 3.03E−04 | −0.222336726 | 0.222336726 | loss | 0.367539674 | 0.145202948 | CCDC428 | 37 | 12 |
| cg07670516 | 8.47E−10 | 3.60E−04 | −0.222334318 | 0.222334318 | loss | 0.64108773 | 0.418753412 | ZNF232 | 37 | 17 |
| cg22576165 | 7.77E−10 | 3.30E−04 | −0.222323036 | 0.222323036 | loss | 0.378764643 | 0.156441607 | | 37 | 10 |
| cg26185500 | 1.01E−09 | 4.27E−04 | −0.222320107 | 0.222320107 | loss | 0.859701513 | 0.637381406 | C8orf86 | 37 | 8 |
| cg20972453 | 4.21E−09 | 1.79E−03 | −0.222315854 | 0.222315854 | loss | 0.387629126 | 0.165313272 | GJB6; GJB6; GJB6; GJB6; GJB6 | 37 | 13 |
| cg14230238 | 2.91E−08 | 1.23E−02 | −0.222305662 | 0.222305662 | loss | 0.466083962 | 0.2437783 | RFX2; RFX2 | 37 | 19 |
| cg07280807 | 1.19E−09 | 5.07E−04 | −0.222302549 | 0.222302549 | loss | 0.776357855 | 0.554055306 | | 37 | 14 |
| cg20366110 | 7.13E−10 | 3.03E−04 | −0.222286961 | 0.222286961 | loss | 0.511282891 | 0.288995929 | ANXA13; ANXA13 | 37 | 8 |
| cg00288957 | 7.13E−10 | 3.03E−04 | −0.222269362 | 0.222269362 | loss | 0.728176862 | 0.5059075 | ANKDD1A | 37 | 15 |
| cg12827405 | 1.01E−09 | 4.27E−04 | −0.222249353 | 0.222249353 | loss | 0.766967958 | 0.544718606 | LINC00518 | 37 | 6 |
| cg25145360 | 1.19E−09 | 5.07E−04 | −0.222234985 | 0.222234985 | loss | 0.722824685 | 0.5005897 | ALDH3A1; ALDH3A1 | 37 | 17 |
| cg11071682 | 3.88E−09 | 1.65E−03 | −0.222183447 | 0.222183447 | loss | 0.607256906 | 0.385073459 | SERTAD4 | 37 | 1 |
| cg13501538 | 7.13E−10 | 3.03E−04 | −0.222177442 | 0.222177442 | loss | 0.812657389 | 0.590479947 | | 37 | 6 |
| cg25054390 | 7.13E−10 | 3.03E−04 | −0.222169458 | 0.222169458 | loss | 0.510520575 | 0.288351118 | GPR68; GPRS8 | 37 | 14 |
| cg23229568 | 7.13E−10 | 3.03E−04 | −0.222163418 | 0.222163418 | loss | 0.945350894 | 0.723187476 | UGT1A6; UGT1A4; UGT1A10; | 37 | 2 |
| | | | | | | | | UGT1A8; UGT1A7; UGT1A5; | | |
| | | | | | | | | UGT1A3; UGT1A9; UGT1A6; | | |
| | | | | | | | | LOC100286922; LOC100286922; | | |
| | | | | | | | | LOC100286922 | | |
| cg01872872 | 1.54E−08 | 6.56E−03 | −0.22215171 | 0.22215171 | loss | 0.858566734 | 0.636415024 | LINC00301 | 37 | 11 |
| cg26112457 | 7.13E−10 | 3.03E−04 | −0.222138364 | 0.222138364 | loss | 0.864856117 | 0.642717753 | APOB | 37 | 2 |
| cg08564522 | 2.16E−09 | 9.17E−04 | −0.222130057 | 0.222130057 | loss | 0.87378514 | 0.651655082 | | 37 | 19 |
| cg12921411 | 7.13E−10 | 3.03E−04 | −0.222089706 | 0.222089706 | loss | 0.558339553 | 0.336249847 | SYNPO; SYNPO | 37 | 5 |
| cg25983544 | 7.77E−10 | 3.30E−04 | −0.222088225 | 0.222088225 | loss | 0.754950225 | 0.532862 | PSMA8; PSMA8; PSMA8 | 37 | 18 |
| cg19743522 | 1.19E−09 | 5.07E−04 | −0.222065017 | 0.222065017 | loss | 0.883859023 | 0.661794006 | DTX1 | 37 | 12 |
| cg21750709 | 7.77E−10 | 3.30E−04 | −0.222059157 | 0.222059157 | loss | 0.363948492 | 0.141889335 | DPPA4 | 37 | 3 |
| cg22079902 | 4.21E−09 | 1.79E−03 | −0.222055552 | 0.222055552 | loss | 0.730277081 | 0.508221529 | PRDM9 | 37 | 5 |
| cg17461600 | 9.22E−08 | 3.91E−02 | −0.22203918 | 0.22203918 | loss | 0.707456068 | 0.485416888 | DAB1 | 37 | 1 |
| cg07297242 | 2.69E−08 | 1.14E−02 | −0.222028223 | 0.222028223 | loss | 0.384511353 | 0.16248313 | | 37 | 2 |
| cg10128806 | 7.13E−10 | 3.03E−04 | −0.222021869 | 0.222021869 | loss | 0.891205051 | 0.669183182 | FRAS1; FRAS1 | 37 | 4 |
| cg18161890 | 1.01E−09 | 4.27E−04 | −0.222019155 | 0.222019155 | loss | 0.428204255 | 0.2061851 | KRT72; KRT72; KRT72; KRT72; | 37 | 12 |
| | | | | | | | | KRT72; KRT72 | | |
| cg09870137 | 7.13E−10 | 3.03E−04 | −0.221996328 | 0.221996328 | loss | 0.508590087 | 0.286693759 | WNT7B | 37 | 22 |
| cg05196187 | 7.13E−10 | 3.03E−04 | −0.221995795 | 0.221995795 | loss | 0.736946972 | 0.514951176 | | 37 | 10 |
| cg13613891 | 1.10E−09 | 4.65E−04 | −0.221995766 | 0.221995766 | loss | 0.876316208 | 0.654320441 | ZBTB47 | 37 | 3 |
| cg06538334 | 8.80E−09 | 3.74E−03 | −0.221972049 | 0.221972049 | loss | 0.602629708 | 0.380657659 | | 37 | X |
| cg12585943 | 7.13E−10 | 3.03E−04 | −0.22196575 | 0.22196575 | loss | 0.361861894 | 0.139896145 | PPT2; PPT2; PPT2; LOC100507547; | 37 | 6 |
| | | | | | | | | LOC100507547; LOC100507547; | | |
| | | | | | | | | LOC100507547; | | |
| | | | | | | | | PPT2-EGFL8 | | |
| cg07912161 | 1.83E−09 | 7.75E−04 | −0.221960776 | 0.221960776 | loss | 0.776693311 | 0.554732535 | PI16; PI16 | 37 | 6 |
| cg09415955 | 7.13E−10 | 3.03E−04 | −0.221872236 | 0.221872236 | loss | 0.602574277 | 0.380702041 | CASZ1; CASZ1 | 37 | 1 |
| cg06349050 | 1.01E−09 | 4.27E−04 | −0.221870521 | 0.221870521 | loss | 0.720461826 | 0.498591306 | SPSB1 | 37 | 1 |
| cg10195901 | 7.77E−10 | 3.30E−04 | −0.221859625 | 0.221859625 | loss | 0.403744704 | 0.181885079 | HORMAD2 | 37 | 22 |
| cg05394244 | 1.10E−09 | 4.65E−04 | −0.221809072 | 0.221809072 | loss | 0.482071155 | 0.260262082 | PTP4A1 | 37 | 6 |
| cg19825600 | 3.40E−08 | 1.44E−02 | −0.221805468 | 0.221805468 | loss | 0.472473415 | 0.250667947 | ALLC | 37 | 2 |
| cg00078861 | 2.65E−08 | 1.12E−02 | −0.221784196 | 0.221784196 | loss | 0.765157425 | 0.543373229 | | 37 | 5 |
| cg07405121 | 1.10E−09 | 4.65E−04 | −0.221766396 | 0.221766396 | loss | 0.497147991 | 0.275381594 | SLC9A3R2; SLC9A3R2; SLC9A3R2; | 37 | 16 |
| | | | | | | | | SLC9A3R2; SLC9A3R2 | | |
| cg15407819 | 7.13E−10 | 3.03E−04 | −0.22175257 | 0.22175257 | loss | 0.925394211 | 0.703641641 | SPTBN4; SPTSN4 | 37 | 19 |
| cg01055696 | 7.13E−10 | 3.03E−04 | −0.221751918 | 0.221751918 | loss | 0.747386436 | 0.525634518 | | 37 | 6 |
| cg08930881 | 5.85E−09 | 2.49E−03 | −0.221685802 | 0.221685802 | loss | 0.781733549 | 0.560047747 | CRTAC1; CRTAC1 | 37 | 10 |
| cg09473826 | 2.73E−09 | 1.18E−03 | −0.221670328 | 0.221670328 | loss | 0.68161824 | 0.459947912 | TNR | 37 | 1 |
| cg14706475 | 1.10E−09 | 4.65E−04 | −0.221662188 | 0.221662188 | loss | 0.803227958 | 0.581565771 | | 37 | 6 |
| cg01298758 | 1.19E−09 | 5.07E−04 | −0.221648426 | 0.221648426 | loss | 0.671576038 | 0.449927612 | USP47; USP47 | 37 | 11 |
| cg15148078 | 7.13E−10 | 3.03E−04 | −0.221646517 | 0.221646517 | loss | 0.670480917 | 0.4488344 | C19orf77; C19orf77 | 37 | 19 |
| cg10949576 | 7.13E−10 | 3.03E−04 | −0.221636886 | 0.221636886 | loss | 0.677379104 | 0.455742218 | PPP1R32; PPP1R32 | 37 | 11 |
| cg25789721 | 9.22E−08 | 3.91E−02 | −0.221631361 | 0.221631361 | loss | 0.579593555 | 0.357962194 | ATP2B2; ATP2B2 | 37 | 3 |
| cg23081026 | 1.10E−09 | 4.65E−04 | −0.221630749 | 0.221630749 | loss | 0.624740896 | 0.403110147 | | 37 | 1 |
| cg17811845 | 1.19E−09 | 5.07E−04 | −0.22161552 | 0.22161552 | loss | 0.649853785 | 0.428238265 | GTF2B | 37 | 1 |
| cg03252786 | 8.47E−10 | 3.60E−04 | −0.22159461 | 0.22159461 | loss | 0.957394487 | 0.735799876 | PKNOX2 | 37 | 11 |
| cg00937742 | 7.13E−10 | 3.03E−04 | −0.22158146 | 0.22158146 | loss | 0.579300208 | 0.357718747 | LYPD1; LYPD1 | 37 | 2 |
| cg20237610 | 7.34E−08 | 3.12E−02 | −0.221549411 | 0.221549411 | loss | 0.477390964 | 0.255841553 | LAMA2; LAMA2 | 37 | 6 |
| cg25844605 | 3.40E−08 | 1.44E−02 | −0.221549059 | 0.221549059 | loss | 0.899864142 | 0.678315082 | LINC00654 | 37 | 20 |
| cg19499085 | 7.84E−10 | 3.33E−04 | −0.221524152 | 0.221524152 | loss | 0.597199946 | 0.375675794 | ATP8B3; ATP8B3; ATP8B3 | 37 | 19 |
| cg16643603 | 2.65E−08 | 1.12E−02 | −0.221514971 | 0.221514971 | loss | 0.59050016 | 0.368985188 | PDE1B; PDE1B | 37 | 12 |
| cg11194708 | 1.02E−09 | 4.33E−04 | −0.221401364 | 0.221401364 | loss | 0.646297723 | 0.424896359 | PACRG; PACRG; PACRG | 37 | 6 |
| cg20249210 | 1.10E−09 | 4.65E−04 | −0.22139848 | 0.22139848 | loss | 0.598672557 | 0.377274076 | | 37 | 12 |
| cg04820679 | 3.12E−09 | 1.32E−03 | −0.221395782 | 0.221395782 | loss | 0.853416529 | 0.632020747 | COL11A2; COL11A2; COL11A2 | 37 | 6 |
| cg00381541 | 5.01E−08 | 2.13E−02 | −0.221395392 | 0.221395392 | loss | 0.525335098 | 0.303939706 | | 37 | 17 |
| cg15554005 | 1.54E−08 | 6.56E−03 | −0.221373083 | 0.221373083 | loss | 0.888428083 | 0.667055 | | 37 | 11 |
| cg09970023 | 7.77E−10 | 3.30E−04 | −0.221367468 | 0.221367468 | loss | 0.532415815 | 0.311048347 | SIX4 | 37 | 14 |
| cg24688803 | 7.13E−10 | 3.03E−04 | −0.221363346 | 0.221363346 | loss | 0.785185075 | 0.563821729 | ALDH1L2; ALDH1L2 | 37 | 12 |
| cg14830003 | 7.13E−10 | 3.03E−04 | −0.221356067 | 0.221356067 | loss | 0.797531096 | 0.576175029 | DNALI1 | 37 | 1 |
| cg14573817 | 7.13E−10 | 3.03E−04 | −0.22135 | 0.22135 | loss | 0.782581547 | 0.561231547 | C12orf23 | 37 | 12 |
| cg20703671 | 7.13E−10 | 3.03E−04 | −0.221349596 | 0.221349596 | loss | 0.437459343 | 0.216109747 | MYO1C; MYO1C; MYO1C | 37 | 17 |
| cg03665132 | 7.13E−10 | 3.03E−04 | −0.221332573 | 0.221332573 | loss | 0.533228749 | 0.311896176 | C1orfS6; C1orf86 | 37 | 1 |
| cg25341032 | 3.97E−08 | 1.69E−02 | −0.22131641 | 0.22131641 | loss | 0.894249804 | 0.672933394 | ADD3; ADD3-AS1 | 37 | 10 |
| cg18048071 | 1.99E−09 | 8.43E−04 | −0.221307038 | 0.221307038 | loss | 0.535623279 | 0.314316241 | VIPR2 | 37 | 7 |
| cg16533838 | 7.13E−10 | 3.03E−04 | −0.221260217 | 0.221260217 | loss | 0.461376017 | 0.2401158 | | 37 | 11 |
| cg23079808 | 1.42E−09 | 6.01E−04 | −0.221255369 | 0.221255369 | loss | 0.363804536 | 0.142549166 | LTBR; LTBR | 37 | 12 |
| cg18628483 | 7.84E−10 | 3.33E−04 | −0.221249393 | 0.221249393 | loss | 0.720949269 | 0.499699876 | KIF5A | 37 | 12 |
| cg03995300 | 2.16E−09 | 9.17E−04 | −0.221233228 | 0.221233228 | loss | 0.417798119 | 0.196564891 | ZNF232 | 37 | 17 |
| cg04769341 | 7.13E−10 | 3.03E−04 | −0.22121609 | 0.22121609 | loss | 0.54849926 | 0.327283171 | TCEAL7 | 37 | X |
| cg03812309 | 2.16E−09 | 9.17E−04 | −0.221199214 | 0.221199214 | loss | 0.742630732 | 0.521431518 | | 37 | 1 |
| cg00537210 | 1.42E−09 | 6.01E−04 | −0.221173126 | 0.221173126 | loss | 0.873689632 | 0.652516506 | KLHL29 | 37 | 2 |
| cg12308391 | 4.01E−09 | 1.70E−03 | −0.221156306 | 0.221156306 | loss | 0.857417688 | 0.636261382 | NCAM1; NCAM1; NCAM1; | 37 | 11 |
| | | | | | | | | NCAM1-AS1 | | |
| cg18221580 | 1.03E−08 | 4.39E−03 | −0.221150913 | 0.221150913 | loss | 0.750083666 | 0.528932753 | SUCLA2 | 37 | 13 |
| cg13551510 | 7.13E−10 | 3.03E−04 | −0.221137115 | 0.221137115 | loss | 0.921596162 | 0.700459047 | NTM | 37 | 11 |
| cg08248579 | 9.22E−08 | 3.91E−02 | −0.221106257 | 0.221106257 | loss | 0.599469457 | 0.3783632 | KIF13A; KIF13A; KIF13A; KIF13A; | 37 | 6 |
| | | | | | | | | KIF13A | | |
| cg07229001 | 1.83E−09 | 7.75E−04 | −0.221101452 | 0.221101452 | loss | 0.817151428 | 0.596049976 | | 37 | 15 |
| cg13540341 | 7.13E−10 | 3.03E−04 | −0.221090026 | 0.221090026 | loss | 0.743162449 | 0.522072424 | MAMSTR; MAMSTR | 37 | 19 |
| cg17937182 | 7.77E−10 | 3.30E−04 | −0.22107157 | 0.22107157 | loss | 0.929279564 | 0.708207994 | PDE4A; PDE4A; PDE4A; PDE4A; | 37 | 19 |
| | | | | | | | | PDE4A | | |
| cg23067299 | 7.77E−10 | 3.30E−04 | −0.221054217 | 0.221054217 | loss | 0.91308167 | 0.692027453 | AHRR; AHRR | 37 | 5 |
| cg05221720 | 8.50E−09 | 3.61E−03 | −0.221052691 | 0.221052691 | loss | 0.860525856 | 0.639473165 | COL9A1; COL9A1 | 37 | 6 |
| cg01181681 | 1.42E−09 | 6.01E−04 | −0.221018328 | 0.221018328 | loss | 0.442450234 | 0.221431906 | TLR5 | 37 | 1 |
| cg00461236 | 9.23E−10 | 3.92E−04 | −0.221016832 | 0.221016832 | loss | 0.852336191 | 0.631319359 | | 37 | 16 |
| cg18810310 | 7.13E−10 | 3.03E−04 | −0.221015627 | 0.221015627 | loss | 0.499491074 | 0.278475447 | | 37 | 14 |
| cg11421972 | 7.13E−10 | 3.03E−04 | −0.220943807 | 0.220943807 | loss | 0.83091963 | 0.609975824 | HSPB7 | 37 | 1 |
| cg14186349 | 3.14E−08 | 1.33E−02 | −0.220931707 | 0.220931707 | loss | 0.586519219 | 0.365587512 | | 37 | 15 |
| cg14352032 | 7.13E−10 | 3.03E−04 | −0.220921954 | 0.220921954 | loss | 0.56829146 | 0.347369506 | | 37 | 17 |
| cg16426764 | 2.30E−08 | 9.75E−03 | −0.220891173 | 0.220891173 | loss | 0.638898785 | 0.418007612 | LHPP; LHPP | 37 | 10 |
| cg21171625 | 7.13E−10 | 3.03E−04 | −0.220861437 | 0.220861437 | loss | 0.534249649 | 0.313388212 | DNAL1 | 37 | 1 |
| cg16302441 | 7.13E−10 | 3.03E−04 | −0.220835275 | 0.220835275 | loss | 0.498229498 | 0.277394224 | POMC; POMC | 37 | 2 |
| cg27384918 | 7.13E−10 | 3.03E−04 | −0.220832991 | 0.220832991 | loss | 0.698186826 | 0.477353835 | | 37 | 16 |
| cg11581248 | 4.21E−09 | 1.79E−03 | −0.220826517 | 0.220826517 | loss | 0.744253411 | 0.523426894 | | 37 | 1 |
| cg27414481 | 7.13E−10 | 3.03E−04 | −0.220821533 | 0.220821533 | loss | 0.768403568 | 0.547582035 | PRKCDBP | 37 | 11 |
| cg08787968 | 9.23E−10 | 3.92E−04 | −0.220804179 | 0.220804179 | loss | 0.558509243 | 0.337705065 | WT1; WT1; WT1; WT1; WT1 | 37 | 11 |
| cg07969646 | 1.10E−09 | 4.65E−04 | −0.220793028 | 0.220793028 | loss | 0.542458445 | 0.321665418 | IER5 | 37 | 1 |
| cg10207181 | 7.13E−10 | 3.03E−04 | −0.220785239 | 0.220785239 | loss | 0.554734815 | 0.333949576 | NBPF8; NBPF8; NBPF8 | 37 | 1 |
| cg04558175 | 7.13E−10 | 3.03E−04 | −0.22078513 | 0.22078513 | loss | 0.763137883 | 0.542352753 | RCN3 | 37 | 19 |
| cg01667892 | 9.54E−09 | 4.05E−03 | −0.220774676 | 0.220774676 | loss | 0.674565423 | 0.453790747 | PRDM9 | 37 | 5 |
| cg21561315 | 1.57E−09 | 6.68E−04 | −0.220752951 | 0.220752951 | loss | 0.959321487 | 0.738568535 | | 37 | 12 |
| cg18105725 | 6.35E−09 | 2.70E−03 | −0.220705003 | 0.220705003 | loss | 0.572345232 | 0.351640229 | SLC22A1; SLC22A1 | 37 | 6 |
| cg12322672 | 7.48E−09 | 3.18E−03 | −0.220661033 | 0.220661033 | loss | 0.871490421 | 0.650829388 | KIRREL3; KIRREL3 | 37 | 11 |
| cg04035597 | 6.30E−08 | 2.65E−02 | −0.220653066 | 0.220653066 | loss | 0.719244608 | 0.498591541 | | 37 | 8 |
| cg11245990 | 7.13E−10 | 3.03E−04 | −0.220640786 | 0.220640786 | loss | 0.459717757 | 0.239076971 | | 37 | 11 |
| cg19567193 | 7.13E−10 | 3.03E−04 | −0.220630411 | 0.220630411 | loss | 0.724929628 | 0.504299218 | RBPMS2 | 37 | 15 |
| cg21747160 | 7.13E−10 | 3.03E−04 | −0.220611555 | 0.220613555 | loss | 0.433940855 | 0.2133293 | LMBR1L | 37 | 12 |
| cg00730441 | 7.13E−10 | 3.03E−04 | −0.22060324 | 0.22060324 | loss | 0.808507392 | 0.587904153 | TBX2 | 37 | 17 |
| cg21700511 | 7.13E−10 | 3.03E−04 | −0.220586424 | 0.220586424 | loss | 0.600519947 | 0.379933524 | HSPA12B; HSPA12B | 37 | 20 |
| cg01451808 | 2.48E−08 | 1.05E−02 | −0.220580132 | 0.220580132 | loss | 0.861954938 | 0.641374806 | | 37 | 3 |
| cg18651192 | 4.57E−09 | 1.94E−03 | −0.220570162 | 0.220570162 | loss | 0.759305009 | 0.538734847 | | 37 | 3 |
| cg24127506 | 8.47E−10 | 3.60E−04 | −0.220549043 | 0.220549043 | loss | 0.876509766 | 0.655960724 | | 37 | 14 |
| cg20254763 | 6.89E−09 | 2.93E−03 | −0.220536371 | 0.220536371 | loss | 0.663858847 | 0.443322476 | | 37 | 1 |
| cg21350413 | 7.13E−10 | 3.03E−04 | −0.22048349 | 0.22048349 | loss | 0.495982943 | 0.275499453 | | 37 | 6 |
| cg18005180 | 7.77E−10 | 3.30E−04 | −0.220475811 | 0.220475811 | loss | 0.617715458 | 0.397239647 | | 37 | 3 |
| cg13073224 | 3.97E−08 | 1.69E−02 | −0.220463967 | 0.220463967 | loss | 0.324925421 | 0.104461454 | | 37 | 12 |
| cg01344644 | 1.01E−09 | 4.27E−04 | −0.220459231 | 0.220459231 | loss | 0.798479372 | 0.578020141 | | 37 | 13 |
| cg07882059 | 1.68E−09 | 7.12E−04 | −0.220455133 | 0.220455133 | loss | 0.481774304 | 0.261319171 | LOC338694 | 37 | 11 |
| cg03726858 | 7.13E−10 | 3.03E−04 | −0.220435678 | 0.220435678 | loss | 0.766347055 | 0.545911376 | | 37 | 17 |
| cg15704304 | 6.30E−08 | 2.68E−02 | −0.220386527 | 0.220386527 | loss | 0.861456068 | 0.641069541 | | 37 | 12 |
| cg06925882 | 7.12E−10 | 3.02E−04 | −0.22037085 | 0.22037085 | loss | 0.986776762 | 0.766405912 | | 37 | 16 |
| cg04306069 | 7.77E−10 | 3.30E−04 | −0.220363554 | 0.220363554 | loss | 0.644231277 | 0.423867724 | | 37 | 5 |
| cg03870261 | 1.12E−08 | 4.76E−03 | −0.220343112 | 0.220343112 | loss | 0.855645847 | 0.635302735 | TIMM13; LMNB2 | 37 | 19 |
| cg06692785 | 7.13E−10 | 3.03E−04 | −0.220341649 | 0.220341649 | loss | 0.546727602 | 0.326385953 | KIAA1614 | 37 | 1 |
| cg06159896 | 7.77E−10 | 3.30E−04 | −0.220295842 | 0.220295842 | loss | 0.387493972 | 0.167198129 | | 37 | 12 |
| cg01692379 | 7.13E−10 | 3.03E−04 | −0.22029087 | 0.22029087 | loss | 0.528114206 | 0.307823335 | RNF39; RNF39 | 37 | 6 |
| cg09943102 | 1.83E−09 | 7.75E−04 | −0.220287563 | 0.220287563 | loss | 0.542628104 | 0.322340541 | | 37 | 7 |
| cg21559386 | 7.13E−10 | 3.03E−04 | −0.220271343 | 0.220271343 | loss | 0.731117502 | 0.510846159 | HNF4A; HNF4A; HNF4A; HNF4A; | 37 | 20 |
| | | | | | | | | HNF4A; HNF4A; HNF4A | | |
| cg05702280 | 1.68E−09 | 7.12E−04 | −0.220239286 | 0.220239286 | loss | 0.721520109 | 0.501280824 | DNAJB5; DNAJB5 | 37 | 9 |
| cg26629730 | 7.13E−10 | 3.03E−04 | −0.220226172 | 0.220226172 | loss | 0.720589066 | 0.500362894 | SCUBE1 | 37 | 22 |
| cg11024728 | 1.83E−09 | 7.75E−04 | −0.220139446 | 0.220139446 | loss | 0.636880228 | 0.416740782 | | 37 | 7 |
| cg03760518 | 7.13E−10 | 3.03E−04 | −0.220104354 | 0.220104354 | loss | 0.494640725 | 0.274536371 | TP73; TP73; TP73; TP73; TP73; | 37 | 1 |
| | | | | | | | | TP73; TP73; TP73; TP73; | | |
| | | | | | | | | TP73; TP73; TP73; TP73 | | |
| cg14006677 | 8.47E−10 | 3.60E−04 | −0.22009148 | 0.22009148 | loss | 0.428069168 | 0.207977688 | DIXDC1; DIXDC1 | 37 | 11 |
| cg24195486 | 7.77E−10 | 3.30E−04 | −0.22005376 | 0.22005376 | loss | 0.557106466 | 0.337052706 | SLC24A4; SLC24A4 | 37 | 14 |
| cg21464975 | 2.16E−09 | 9.17E−04 | −0.219993745 | 0.219993745 | loss | 0.907662845 | 0.6876691 | MARCH10; MARCH10; MIR548W | 37 | 17 |
| cg01682285 | 2.30E−08 | 9.75E−03 | −0.219973475 | 0.219973475 | loss | 0.469557987 | 0.249584512 | TSHR; TSHR; TSHR | 37 | 14 |
| cg16212279 | 7.13E−10 | 3.03E−04 | −0.219915982 | 0.219915982 | loss | 0.555036306 | 0.335120324 | | 37 | 11 |
| cg02269978 | 3.57E−09 | 1.52E−03 | −0.21990471 | 0.21990471 | loss | 0.485691998 | 0.265787288 | | 37 | 6 |
| cg07841866 | 7.13E−10 | 3.03E−04 | −0.219869562 | 0.219869562 | loss | 0.867366174 | 0.647496612 | NOS3 | 37 | 7 |
| cg07058988 | 3.12E−09 | 1.32E−03 | −0.219850387 | 0.219850387 | loss | 0.827245552 | 0.607395165 | | 37 | 17 |
| cg10729496 | 7.13E−10 | 3.03E−04 | −0.21984022 | 0.21984022 | loss | 0.941591796 | 0.721751576 | FANCD2OS; FANCD2OS; FANCD2OS; | 37 | 3 |
| | | | | | | | | FANCD2OS | | |
| cg00080125 | 7.13E−10 | 3.03E−04 | −0.219838844 | 0.219838844 | loss | 0.502035126 | 0.282196282 | C17orf82 | 37 | 17 |
| cg11051752 | 1.01E−09 | 4.27E−04 | −0.219832992 | 0.219832992 | loss | 0.879910351 | 0.660077359 | | 37 | 6 |
| cg03129014 | 8.47E−10 | 3.60E−04 | −0.219823469 | 0.219823469 | loss | 0.475395145 | 0.255571676 | GJA3 | 37 | 13 |
| cg01055562 | 8.47E−10 | 3.60E−04 | −0.219808924 | 0.219808924 | loss | 0.470195777 | 0.250386853 | COG2; COG2 | 37 | 1 |
| cg04839207 | 9.23E−10 | 3.92E−04 | −0.219801935 | 0.219801935 | loss | 0.6705141 | 0.450712165 | SERTAD4 | 37 | 1 |
| cg19362874 | 5.39E−09 | 2.29E−03 | −0.219768356 | 0.219768356 | loss | 0.850664332 | 0.630895976 | | 37 | 6 |
| cg09361493 | 1.01E−09 | 4.27E−04 | −0.219751928 | 0.219751928 | loss | 0.350149402 | 0.130397474 | | 37 | 2 |
| cg18990042 | 7.13E−10 | 3.03E−04 | −0.219741618 | 0.219741618 | loss | 0.334662487 | 0.114920869 | COL2A1; COL2A1 | 37 | 12 |
| cg22618405 | 7.13E−10 | 3.03E−04 | −0.219726405 | 0.219726405 | loss | 0.960683175 | 0.740956771 | | 37 | 2 |
| cg08289525 | 1.01E−09 | 4.27E−04 | −0.219707697 | 0.219707697 | loss | 0.464885379 | 0.245177682 | ST8SIA2 | 37 | 15 |
| cg00474218 | 7.13E−10 | 3.03E−04 | −0.219660388 | 0.219660388 | loss | 0.579333094 | 0.359672706 | | 37 | 15 |
| cg03353445 | 4.57E−09 | 1.94E−03 | −0.219658291 | 0.219658291 | loss | 0.650910379 | 0.431252088 | GPRC5A | 37 | 12 |
| cg18246298 | 7.13E−10 | 3.03E−04 | 0.219657585 | 0.219657585 | loss | 0.823980121 | 0.604322535 | C11orf53 | 37 | 11 |
| cg02914827 | 1.99E−09 | 8.43E−04 | −0.219647751 | 0.219647751 | loss | 0.643932592 | 0.424284841 | MMP21 | 37 | 10 |
| cg04122918 | 7.13E−10 | 3.03E−04 | −0.219539124 | 0.219539124 | loss | 0.919827094 | 0.700287971 | DPF3; DPF3; DPF3; DPF3 | 37 | 14 |
| cg21875655 | 2.41E−09 | 1.03E−03 | −0.2195298 | 0.2195298 | loss | 0.796815423 | 0.577285624 | | 37 | 6 |
| cg26047066 | 7.13E−10 | 3.03E−04 | −0.219522315 | 0.219522315 | loss | 0.978643709 | 0.759121394 | | 37 | 1 |
| cg04052235 | 9.23E−10 | 3.92E−04 | −0.219514801 | 0.219514801 | loss | 0.451381836 | 0.231867035 | | 37 | 11 |
| cg07291889 | 8.47E−10 | 3.60E−04 | −0.219478633 | 0.219478633 | loss | 0.801387562 | 0.581908929 | | 37 | 8 |
| cg03290213 | 2.16E−09 | 9.17E−04 | −0.219476022 | 0.219476022 | loss | 0.908549028 | 0.689073006 | CARD14; CARD14; CARD14 | 37 | 17 |
| cg01422467 | 5.39E−09 | 2.29E−03 | −0.219453999 | 0.219453999 | loss | 0.614248023 | 0.394794024 | SLC9B2; SLC9B2 | 37 | 4 |
| cg23234622 | 2.35E−09 | 9.98E−04 | −0.219390498 | 0.219390498 | loss | 0.916892262 | 0.697501765 | | 37 | 2 |
| cg06159404 | 3.67E−08 | 1.56E−02 | −0.219383414 | 0.219383414 | loss | 0.3269962 | 0.107612786 | | 37 | 10 |
| cg08284496 | 8.47E−10 | 3.60E−04 | −0.219367561 | 0.219367561 | loss | 0.495599791 | 0.276232229 | GRB7; GRB7; GRB7 | 37 | 17 |
| cg08933517 | 1.43E−08 | 6.06E−03 | −0.219350312 | 0.219350312 | loss | 0.808104283 | 0.588753971 | TDRD9 | 37 | 14 |
| cg18936052 | 7.13E−10 | 3.03E−04 | −0.219346465 | 0.219346465 | loss | 0.546545653 | 0.327199188 | FAM76A; FAM76A; FAM76A; | 37 | 1 |
| | | | | | | | | FAM76A; FAM76A | | |
| cg26143452 | 2.16E−09 | 9.17E−04 | −0.219287255 | 0.219287255 | loss | 0.375263643 | 0.155976389 | TBPL2; TBPL2 | 37 | 14 |
| cg19214331 | 7.13E−10 | 3.03E−04 | −0.21927179 | 0.21927179 | loss | 0.898020749 | 0.678748959 | MYL9; MYL9 | 37 | 20 |
| cg03475293 | 7.13E−10 | 3.03E−04 | −0.219267892 | 0.219267892 | loss | 0.957051698 | 0.737783806 | | 37 | 6 |
| cg11941920 | 1.10E−09 | 4.65E−04 | −0.219262124 | 0.219262124 | loss | 0.398104762 | 0.178842638 | | 37 | 6 |
| cg01267165 | 7.13E−10 | 3.03E−04 | −0.219261973 | 0.219261973 | loss | 0.816018085 | 0.596756112 | LITAF; LITAF; LITAF; LITAF | 37 | 16 |
| cg15393702 | 3.28E−09 | 1.39E−03 | −0.219218197 | 0.219218197 | loss | 0.633303721 | 0.414085524 | ANKRD29 | 37 | 18 |
| cg22108980 | 7.13E−10 | 3.03E−04 | −0.219214761 | 0.219214761 | loss | 0.403814832 | 0.184600071 | IGFBP1 | 37 | 7 |
| cg07038187 | 7.13E−10 | 3.03E−04 | −0.219189773 | 0.219189773 | loss | 0.876774926 | 0.657585153 | LINC00925 | 37 | 15 |
| cg02873391 | 1.19E−09 | 5.07E−04 | −0.219189213 | 0.219189213 | loss | 0.802498008 | 0.583308794 | | 37 | 11 |
| cg04995311 | 9.23E−10 | 3.92E−04 | −0.219187324 | 0.219187324 | loss | 0.799880489 | 0.580693165 | USP29 | 37 | 19 |
| cg15249221 | 7.13E−10 | 3.03E−04 | −0.219176968 | 0.219176968 | loss | 0.613955168 | 0.3947782 | | 37 | 7 |
| cg20852302 | 7.13E−10 | 3.03E−04 | −0.2191747 | 0.2191747 | loss | 0.548334089 | 0.329159388 | | 37 | 20 |
| cg11814882 | 7.13E−10 | 3.03E−04 | −0.219169087 | 0.219169087 | loss | 0.852040181 | 0.632871094 | CYGB; PRCD | 37 | 17 |
| cg09981929 | 1.22E−08 | 5.16E−03 | −0.219138213 | 0.219138213 | loss | 0.350564706 | 0.131426492 | | 37 | 10 |
| cg17247293 | 7.77E−10 | 3.30E−04 | −0.219135236 | 0.219135236 | loss | 0.665769336 | 0.4466341 | C6orf123 | 37 | 6 |
| cg19460817 | 8.47E−10 | 3.60E−04 | −0.219113959 | 0.219113959 | loss | 0.622707436 | 0.403593476 | CELF1 | 37 | 11 |
| cg23579062 | 7.13E−10 | 3.03E−04 | −0.219089932 | 0.219089932 | loss | 0.596580296 | 0.377490365 | FAM219A; FAM219A; FAM219A; | 37 | 9 |
| | | | | | | | | FAM219A; FAM219A; | | |
| | | | | | | | | DNAI1; DNAI1; FAM219A | | |
| cg19797049 | 2.35E−09 | 9.98E−04 | −0.219041161 | 0.219041161 | loss | 0.634095232 | 0.415054071 | DNAJC2; DNAJC2; DNAJC2; | 37 | 7 |
| | | | | | | | | DNAJC2 | | |
| cg00990380 | 7.13E−10 | 3.03E−04 | −0.219033945 | 0.219033945 | loss | 0.666096745 | 0.4470628 | | 37 | 6 |
| cg09514174 | 7.13E−10 | 3.03E−04 | −0.219021132 | 0.219021132 | loss | 0.530090796 | 0.311069665 | OSCP1; OSCP1 | 37 | 1 |
| cg18143792 | 7.13E−10 | 3.03E−04 | −0.218995762 | 0.218995762 | loss | 0.459458026 | 0.240462265 | | 37 | 1 |
| cg08252303 | 8.11E−09 | 3.44E−03 | −0.218974178 | 0.218974178 | loss | 0.763207555 | 0.544233376 | KCNMA1; KCNMA1; KCNMA1; | 37 | 10 |
| | | | | | | | | KCNMA1; KCNMA1; KCNMA1; | | |
| | | | | | | | | KCNMA1; KCNMA1; | | |
| | | | | | | | | KCNMA1 | | |
| cg17184245 | 7.13E−10 | 3.03E−04 | −0.218968596 | 0.218968596 | loss | 0.656377549 | 0.437408953 | RAP1GAP; RAP1GAP | 37 | 1 |
| cg11651446 | 7.13E−10 | 3.03E−04 | −0.218960237 | 0.218960237 | loss | 0.742304049 | 0.523343812 | IL22RA1 | 37 | 1 |
| cg04467589 | 7.13E−10 | 3.03E−04 | −0.218944933 | 0.218944933 | loss | 0.685363509 | 0.466418576 | RNF39; RNF39 | 37 | 6 |
| cg18227471 | 7.13E−10 | 3.03E−04 | −0.218921942 | 0.218921942 | loss | 0.75993383 | 0.541011888 | NKX6-3 | 37 | 8 |
| cg17974424 | 3.28E−09 | 1.39E−03 | −0.21889955 | 0.21889955 | loss | 0.616019874 | 0.397120324 | | 37 | 6 |
| cg11750736 | 6.30E−08 | 2.68E−02 | −0.218862058 | 0.218862058 | loss | 0.695424164 | 0.476562106 | TMEM220; TMEM220- | 37 | 17 |
| | | | | | | | | AS1; TMEM220- | | |
| | | | | | | | | AS1; TMEM220- | | |
| | | | | | | | | AS1; TMEM220-AS1 | | |
| cg18022883 | 7.13E−10 | 3.03E−04 | −0.218841768 | 0.218841768 | loss | 0.807123021 | 0.588281253 | | 37 | 4 |
| cg00035416 | 8.47E−10 | 3.60E−04 | −0.218835162 | 0.218835162 | loss | 0.406621115 | 0.187785953 | STAU2; STAU2; STAU2; STAU2; | 37 | 8 |
| | | | | | | | | STAU2; STAU2; STAU2 | | |
| cg09511963 | 1.01E−09 | 4.27E−04 | −0.218779099 | 0.218779099 | loss | 0.442404096 | 0.223624998 | LOC494141; LOC494141; LOC494141 | 37 | 11 |
| cg13305336 | 5.85E−09 | 2.49E−03 | −0.218774293 | 0.218774293 | loss | 0.751596217 | 0.532821924 | | 37 | 1 |
| cg26344606 | 7.13E−10 | 3.03E−04 | −0.218766283 | 0.218766283 | loss | 0.364928625 | 0.146162342 | HR; HR | 37 | 8 |
| cg18059464 | 7.13E−10 | 3.03E−04 | −0.21875644 | 0.21875644 | loss | 0.602763134 | 0.384006694 | GPR123 | 37 | 10 |
| cg23831927 | 7.13E−10 | 3.03E−04 | −0.218746468 | 0.218746468 | loss | 0.883558362 | 0.669821894 | CRYL1 | 37 | 13 |
| cg07850154 | 2.16E−09 | 9.17E−04 | −0.218743719 | 0.218743719 | loss | 0.525792577 | 0.307048859 | RNF180; RNF180 | 37 | 5 |
| cg23651356 | 9.56E−10 | 4.06E−04 | −0.218717175 | 0.218717175 | loss | 0.431183016 | 0.212465841 | RGS17 | 37 | 6 |
| cg12837463 | 7.13E−10 | 3.03E−04 | −0.218697852 | 0.218697852 | loss | 0.596421223 | 0.377723371 | | 37 | 7 |
| cg07929768 | 8.47E−10 | 3.60E−04 | −0.218670862 | 0.218670862 | loss | 0.476740368 | 0.258069506 | | 37 | 2 |
| cg13536265 | 7.13E−10 | 3.03E−04 | −0.218661124 | 0.218661124 | loss | 0.370985583 | 0.152324459 | NFATC4; NFATC4; NFATC4; | 37 | 14 |
| | | | | | | | | NFATC4 | | |
| cg01927921 | 9.23E−10 | 3.92E−04 | −0.218638364 | 0.218638364 | loss | 0.702265828 | 0.483627465 | | 37 | 5 |
| cg12800962 | 7.13E−10 | 3.03E−04 | −0.218615594 | 0.218615594 | loss | 0.660645106 | 0.442029512 | | 37 | 20 |
| cg12594581 | 8.47E−10 | 3.60E−04 | −0.218615541 | 0.218615541 | loss | 0.348507804 | 0.129892262 | MFI2; MFI2 | 37 | 3 |
| cg02747254 | 1.01E−09 | 4.27E−04 | −0.218614476 | 0.218614476 | loss | 0.52847707 | 0.309862594 | KCTD13 | 37 | 16 |
| cg25309888 | 7.13E−10 | 3.03E−04 | −0.218591518 | 0.218591518 | loss | 0.5412194 | 0.322627882 | | 37 | 2 |
| cg12085698 | 1.99E−09 | 8.43E−04 | −0.218584608 | 0.218584608 | loss | 0.842203138 | 0.623618529 | SPON1 | 37 | 11 |
| cg17351974 | 7.13E−10 | 3.03E−04 | −0.218580703 | 0.218580703 | loss | 0.322588464 | 0.104007761 | RPL37 | 37 | 5 |
| cg04463638 | 1.73E−09 | 7.34E−04 | −0.218529531 | 0.218529531 | loss | 0.781962625 | 0.553433094 | CLDN5; CLDN5; CLDN5; CLDN5 | 37 | 22 |
| cg00919055 | 7.13E−10 | 3.03E−04 | −0.218525945 | 0.218525945 | loss | 0.424107062 | 0.205581118 | CDX1 | 37 | 5 |
| cg21853510 | 9.23E−10 | 3.92E−04 | −0.218518478 | 0.218518478 | loss | 0.941541402 | 0.723022924 | SECTM1 | 37 | 17 |
| cg06222397 | 7.13E−10 | 3.03E−04 | −0.218478427 | 0.218478427 | loss | 0.581446168 | 0.362967741 | | 37 | 16 |
| cg24694501 | 1.83E−09 | 7.75E−04 | −0.218474032 | 0.218474032 | loss | 0.479391785 | 0.260917753 | | 37 | 2 |
| cg05965387 | 7.13E−10 | 3.03E−04 | −0.21847279 | 0.21847279 | loss | 0.725791713 | 0.507318924 | DLEU7; DLEU7; DLEU7- | 37 | 13 |
| | | | | | | | | AS1 | | |
| cg03632420 | 2.56E−09 | 1.09E−03 | −0.218461352 | 0.218461352 | loss | 0.599916211 | 0.381454859 | | 37 | 16 |
| cg16708623 | 2.22E−09 | 9.41E−04 | −0.218456546 | 0.218456546 | loss | 0.323020058 | 0.609563512 | TRIM2; TRIM2 | 37 | 4 |
| cg12111783 | 5.41E−08 | 2.30E−02 | −0.218434879 | 0.218434879 | loss | 0.38040686 | 0.161971981 | BCOR; BCOR | 37 | X |
| cg00571021 | 7.84E−10 | 3.33E−04 | −0.218425105 | 0.218425105 | loss | 0.83198524 | 0.613560135 | NOS3 | 37 | 7 |
| cg00435063 | 2.30E−08 | 9.75E−03 | −0.218407199 | 0.218407199 | loss | 0.793068981 | 0.574661782 | PTPRN2; PTPRN2; PTPRN2 | 37 | 7 |
| cg00259472 | 8.80E−09 | 3.74E−03 | −0.218401845 | 0.218401845 | loss | 0.806854898 | 0.588453053 | FABP6; FABP6 | 37 | 5 |
| cg14473643 | 1.01E−09 | 4.27E−04 | −0.218394486 | 0.218394486 | loss | 0.345075632 | 0.126681146 | ZBTB22; TAPBP; ZBTB22; TAPBP; | 37 | 6 |
| | | | | | | | | TAPBP | | |
| cg09928766 | 7.13E−10 | 3.03E−04 | −0.218384071 | 0.218384071 | loss | 0.396767842 | 0.178383771 | IGLON5 | 37 | 19 |
| cg27511413 | 7.13E−10 | 3.03E−04 | −0.218380622 | 0.218380622 | loss | 0.765624392 | 0.547243771 | ASGR1; ASGR1; ASGR1; ASGR1 | 37 | 17 |
| cg03908908 | 7.13E−10 | 3.03E−04 | −0.218365464 | 0.218365464 | loss | 0.59106937 | 0.372703906 | MYO7A; MYO7A; MYO7A | 37 | 11 |
| cg24161925 | 1.19E−09 | 5.07E−04 | −0.218358626 | 0.218358626 | loss | 0.424663191 | 0.206304565 | ECT2L; ECT2L | 37 | 6 |
| cg17792806 | 7.13E−10 | 3.03E−04 | −0.218334322 | 0.218334322 | loss | 0.462768981 | 0.244434659 | TCEAL7 | 37 | X |
| cg16105461 | 7.84E−10 | 3.33E−04 | −0.218328542 | 0.218328542 | loss | 0.850304883 | 0.631976341 | DAGLA | 37 | 11 |
| cg04017102 | 4.57E−09 | 1.94E−03 | −0.218318625 | 0.218318625 | loss | 0.785044102 | 0.566725476 | | 37 | 5 |
| cg03143118 | 7.13E−10 | 3.03E−04 | −0.218296771 | 0.218296771 | loss | 0.819456336 | 0.601159565 | PTCRA; PTCRA; PTCRA; PTCRA | 37 | 6 |
| cg25500028 | 1.30E−09 | 5.52E−04 | −0.218290827 | 0.218290827 | loss | 0.468344309 | 0.250053482 | | 37 | 3 |
| cg13360823 | 7.13E−10 | 3.03E−04 | −0.218285895 | 0.218285895 | loss | 0.331837794 | 0.113551899 | CACNG6; CACNG6; CACNG6; | 37 | 19 |
| | | | | | | | | CACNG6 | | |
| cg13459217 | 8.80E−09 | 3.74E−03 | −0.218267032 | 0.218267032 | loss | 0.957905891 | 0.739638859 | KLHDC8A; KLHDC8A; KLHDC8A; | 37 | 1 |
| | | | | | | | | KLHDC8A | | |
| cg12656497 | 7.13E−10 | 3.03E−04 | −0.218251622 | 0.218251622 | loss | 0.459940645 | 0.241689024 | LINC00111 | 37 | 21 |
| cg05372495 | 7.13E−10 | 3.03E−04 | −0.218217218 | 0.218217218 | loss | 0.945273247 | 0.727056029 | ABCA7 | 37 | 19 |
| cg02540026 | 7.77E−10 | 3.30E−04 | −0.218214016 | 0.218214016 | loss | 0.719064192 | 0.500850176 | | 37 | 2 |
| cg12384004 | 7.77E−10 | 3.30E−04 | −0.218207159 | 0.218207159 | loss | 0.469316742 | 0.251109582 | ATP8A2 | 37 | 13 |
| cg03688324 | 8.47E−10 | 3.60E−04 | −0.218183908 | 0.218183908 | loss | 0.582005943 | 0.363822035 | FGF9 | 37 | 13 |
| cg18648917 | 8.47E−10 | 3.60E−04 | −0.218159408 | 0.218159408 | loss | 0.754246849 | 0.536087441 | | 37 | 3 |
| cg08721802 | 7.13E−10 | 3.03E−04 | −0.218127774 | 0.218127774 | loss | 0.327647611 | 0.109519837 | | 37 | 4 |
| cg24160421 | 7.13E−10 | 3.03E−04 | −0.218091695 | 0.218091695 | loss | 0.39675213 | 0.178660435 | | 37 | 8 |
| cg11606877 | 3.67E−08 | 1.56E−02 | −0.218091028 | 0.218091028 | loss | 0.68916444 | 0.471073412 | TNXB | 37 | 6 |
| cg11152463 | 7.13E−10 | 3.03E−04 | −0.218089934 | 0.218089934 | loss | 0.805765692 | 0.587675759 | | 37 | 1 |
| cg26214742 | 7.77E−10 | 3.30E−04 | −0.218038546 | 0.218038546 | loss | 0.384218151 | 0.166179605 | H2AFY; H2AFY; H2AFY; H2AFY | 37 | 5 |
| cg17156432 | 4.21E−09 | 1.79E−03 | −0.218024215 | 0.218024215 | loss | 0.557318879 | 0.339294665 | | 37 | 15 |
| cg06618097 | 7.13E−10 | 3.03E−04 | −0.218023039 | 0.218023039 | loss | 0.738015545 | 0.519992506 | PPP5D1; PNMAL2 | 37 | 19 |
| cg05871254 | 2.78E−09 | 1.18E−03 | −0.218010041 | 0.218010041 | loss | 0.696250764 | 0.478240724 | HS3ST3B1 | 37 | 17 |
| cg21380024 | 1.10E−09 | 4.65E−04 | −0.218007259 | 0.218007259 | loss | 0.507881447 | 0.289874188 | MIR548W | 37 | 17 |
| cg05751839 | 9.23E−10 | 3.92E−04 | −0.217998952 | 0.217998952 | loss | 0.701300217 | 0.483301265 | SORCS2 | 37 | 4 |
| cg07570257 | 1.26E−08 | 5.33E−03 | −0.217981239 | 0.217981239 | loss | 0.777099302 | 0.559118063 | COL11A2; COL11A2; COL11A2 | 37 | 6 |
| cg09953287 | 7.13E−10 | 3.03E−04 | −0.217973822 | 0.217973822 | loss | 0.451755492 | 0.233781671 | | 37 | 10 |
| cg20371401 | 3.57E−09 | 1.52E−03 | −0.21794874 | 0.21794874 | loss | 0.688776334 | 0.470827594 | C2; C2; C2; C2; C2; C2 | 37 | 6 |
| cg03687084 | 1.68E−09 | 7.12E−04 | −0.217925411 | 0.217925411 | loss | 0.931958523 | 0.714033112 | AHCTF1P1 | 37 | 2 |
| cg18685071 | 7.13E−10 | 3.03E−04 | −0.217897222 | 0.217897222 | loss | 0.734653245 | 0.516756024 | SMKR1 | 37 | 7 |
| cg04231050 | 6.35E−09 | 2.70E−03 | −0.217890055 | 0.217890055 | loss | 0.760739508 | 0.542849453 | BFSP2 | 37 | 3 |
| cg02486415 | 1.19E−09 | 5.07E−04 | −0.217884073 | 0.217884073 | loss | 0.513921238 | 0.296037165 | | 37 | 12 |
| cg25771683 | 4.57E−09 | 1.94E−03 | −0.217799399 | 0.217799399 | loss | 0.431782287 | 0.213982888 | | 37 | 12 |
| cg13308964 | 1.07E−07 | 4.55E−02 | −0.217790032 | 0.217790032 | loss | 0.647587679 | 0.429797647 | | 37 | 17 |
| cg01077274 | 7.13E−10 | 3.03E−04 | −0.217751465 | 0.217751465 | loss | 0.457223142 | 0.239471676 | | 37 | 1 |
| cg26968378 | 2.78E−09 | 1.18E−03 | −0.217747572 | 0.217747572 | loss | 0.40657316 | 0.188825588 | | 37 | 6 |
| cg14278345 | 7.13E−10 | 3.03E−04 | −0.217745158 | 0.217745158 | loss | 0.895975664 | 0.678230506 | IL17REL; IL17REL | 37 | 22 |
| cg06353830 | 9.23E−10 | 3.92E−04 | −0.217710988 | 0.217710988 | loss | 0.914413106 | 0.696702118 | | 37 | 1 |
| cg26180255 | 9.54E−09 | 4.05E−03 | −0.217614557 | 0.217614557 | loss | 0.889062287 | 0.671447729 | CELSR1 | 37 | 22 |
| cg13246592 | 7.13E−10 | 3.03E−04 | −0.217608001 | 0.217608001 | loss | 0.587662277 | 0.370054276 | P2RX5; P2RX5; P2RX5; P2RX5; | 37 | 17 |
| cg03662049 | 7.13E−10 | 3.03E−04 | −0.217607033 | 0.217507033 | loss | 0.539655221 | 0.322048188 | | 37 | 2 |
| cg02359712 | 8.47E−10 | 3.60E−04 | −0.217601718 | 0.217601718 | loss | 0.4678947 | 0.250292982 | | 37 | 5 |
| cg20531550 | 7.13E−10 | 3.03E−04 | −0.217591805 | 0.217591805 | loss | 0.804723523 | 0.587131718 | KCP; KCP; KCP; KCP | 37 | 7 |
| cg19515035 | 7.13E−10 | 3.03E−04 | −0.217591344 | 0.217591344 | loss | 0.296261604 | 0.078670259 | RTKN | 37 | 2 |
| cg07594531 | 3.02E−09 | 1.28E−03 | −0.21755189 | 0.21756189 | loss | 0.650596S08 | 0.433034618 | GJB4 | 37 | 1 |
| cg09834706 | 1.42E−09 | 6.01E−04 | −0.217561691 | 0.217561691 | loss | 0.701374538 | 0.483812847 | | 37 | 2 |
| cg24324584 | 1.01E−09 | 4.27E−04 | −0.217506021 | 0.217506021 | loss | 0.803913409 | 0.586407388 | | 37 | 10 |
| cg09971562 | 3.88E−09 | 1.65E−03 | −0.217462336 | 0.217462336 | loss | 0.57672883 | 0.359266494 | | 37 | 11 |
| cg15681462 | 7.13E−10 | 3.03E−04 | −0.217447932 | 0.217447932 | loss | 0.416160326 | 0.198712394 | IL11; IL11 | 37 | 19 |
| cg22376864 | 9.23E−10 | 3.92E−04 | −0.217441597 | 0.217441597 | loss | 0.600845432 | 0.383403835 | ATP1A3; ATP1A3; ATP1A3; | 37 | 19 |
| | | | | | | | | ATP1A3 | | |
| cg25096368 | 7.13E−10 | 3.03E−04 | −0.217434612 | 0.217434612 | loss | 0.716558606 | 0.499123994 | CISTR-ACT | 37 | 12 |
| cg19473656 | 9.23E−10 | 3.92E−04 | −0.217426792 | 0.217426792 | loss | 0.912957762 | 0.695530971 | SLC7A8; SLC7A8; SLC7A8; SLC7A8; | 37 | 14 |
| | | | | | | | | SLC7A8; SLC7A8; SLC7A8 | | |
| cg23562538 | 7.13E−10 | 3.03E−04 | −0.217424745 | 0.217424745 | loss | 0.621819362 | 0.404394618 | MAPK15 | 37 | 8 |
| cg21656205 | 1.10E−09 | 4.65E−04 | −0.217342303 | 0.217342303 | loss | 0.421988998 | 0.204646695 | RIMS4; RIMS4 | 37 | 20 |
| cg14525458 | 7.13E−10 | 3.03E−04 | −0.217311352 | 0.217311352 | loss | 0.603899428 | 0.386588076 | KDELR1 | 37 | 19 |
| cg22341310 | 9.23E−10 | 3.92E−04 | −0.21729519 | 0.21729519 | loss | 0.325748136 | 0.108452946 | ZNF541 | 37 | 19 |
| cg05654765 | 1.89E−09 | 8.01E−04 | −0.217283412 | 0.217283412 | loss | 0.397446606 | 0.180163194 | LAMB2; LAMB2 | 37 | 3 |
| cg25189241 | 8.47E−10 | 3.60E−04 | −0.217281175 | 0.217281175 | loss | 0.801291175 | 0.58401 | GORASP2; GORASP2 | 37 | 2 |
| cg20248204 | 7.13E−10 | 3.03E−04 | −0.217272038 | 0.217272038 | loss | 0.376236232 | 0.158964194 | KCTD12 | 37 | 13 |
| cg19518672 | 1.42E−09 | 6.01E−04 | −0.217265098 | 0.217265098 | loss | 0.443798657 | 0.226533559 | SEPT8; SEPT8; SEPT8; SEPT8; | 37 | 5 |
| | | | | | | | | SEPT8 | | |
| cg26063874 | 7.13E−10 | 3.03E−04 | −0.217246959 | 0.217246959 | loss | 0.622263547 | 0.405016588 | | 37 | 7 |
| cg17517709 | 1.54E−08 | 6.56E−03 | −0.217246783 | 0.217246783 | loss | 0.881976583 | 0.6647298 | | 37 | 10 |
| cg10851010 | 7.13E−10 | 3.03E−04 | −0.217231511 | 0.217231511 | loss | 0.708748011 | 0.4915165 | | 37 | 17 |
| cg11955878 | 8.47E−10 | 3.60E−04 | −0.217218879 | 0.217218879 | loss | 0.330878185 | 0.113659306 | TRIM61; TRIM61 | 37 | 4 |
| cg03159409 | 1.01E−09 | 4.27E−04 | −0.217201457 | 0.217201457 | loss | 0.867966245 | 0.650764788 | ZNF664- | 37 | 12 |
| | | | | | | | | FAM101A; FAM101A | | |
| cg13607218 | 7.13E−10 | 3.03E−04 | −0.217173749 | 0.217173749 | loss | 0.359217872 | 0.142044122 | | 37 | 2 |
| cg15644727 | 8.56E−10 | 3.63E−04 | −0.217163717 | 0.217163717 | loss | 0.61530854 | 0.398144824 | | 37 | 11 |
| cg22179564 | 2.48E−08 | 1.05E−02 | −0.217159267 | 0.217159267 | loss | 0.515842938 | 0.298683671 | | 37 | 5 |
| cg05290737 | 3.88E−09 | 1.65E−03 | −0.217155492 | 0.217155492 | loss | 0.681410362 | 0.464254871 | HCAR1; HCAR1 | 37 | 12 |
| cg13245431 | 7.13E−10 | 3.03E−04 | −0.217151198 | 0.217151198 | loss | 0.567465251 | 0.350314053 | HORMAD2 | 37 | 22 |
| cg11197905 | 7.13E−10 | 3.03E−04 | −0.217148984 | 0.217148984 | loss | 0.487936708 | 0.270787724 | ZNF5188 | 37 | 4 |
| cg18626035 | 3.02E−09 | 1.28E−03 | −0.217130058 | 0.217130058 | loss | 0.742911564 | 0.525781506 | SLC9B2; SLC9B2 | 37 | 4 |
| cg15922705 | 4.64E−08 | 1.97E−02 | −0.217115575 | 0.217115575 | loss | 0.842214045 | 0.625098471 | COL9A1; COL9A1 | 37 | 6 |
| cg21242079 | 1.68E−09 | 7.12E−04 | −0.217107064 | 0.217107064 | loss | 0.474347758 | 0.257240694 | ADAMTS7 | 37 | 15 |
| cg23028178 | 1.01E−09 | 4.27E−04 | −0.217082864 | 0.217082864 | loss | 0.787310223 | 0.570227359 | NID1 | 37 | 1 |
| cg19097322 | 3.57E−09 | 1.52E−03 | −0.217014915 | 0.217014915 | loss | 0.517729774 | 0.300714859 | FABP7 | 37 | 6 |
| cg07518714 | 1.19E−09 | 5.07E−04 | −0.21700885 | 0.21700885 | loss | 0.615467121 | 0.398458271 | TNXB | 37 | 6 |
| cg19696491 | 3.02E−09 | 1.28E−03 | −0.217007541 | 0.217007541 | loss | 0.467123106 | 0.250115565 | CHRNA5 | 37 | 15 |
| cg16014085 | 7.13E−10 | 3.03E−04 | −0.217007287 | 0.217007287 | loss | 0.520635723 | 0.303628435 | ZNF48; ZNF48; ZNF48; ZNF48 | 37 | 16 |
| cg03871754 | 7.13E−10 | 3.03E−04 | −0.216999329 | 0.216999329 | loss | 0.53473137 | 0.317732041 | | 37 | 17 |
| cg24716416 | 1.83E−09 | 7.75E−04 | −0.216995181 | 0.216995181 | loss | 0.674647375 | 0.457652194 | | 37 | 4 |
| cg13436378 | 1.03E−08 | 4.39E−03 | −0.216990546 | 0.216990546 | loss | 0.744471834 | 0.527481288 | DYSF; DYSF; DYSF; DYSF; DYSF; | 37 | 2 |
| | | | | | | | | DYSF; DYSF; DYSF; DYSF; | | |
| | | | | | | | | DYSF; DYSF; DYSF; DYSF; DYSF | | |
| cg13355424 | 7.13E−10 | 3.03E−04 | −0.216985696 | 0.216985696 | loss | 0.387054008 | 0.170068312 | | 37 | 1 |
| cg21870551 | 7.13E−10 | 3.03E−04 | −0.216969424 | 0.216969424 | loss | 0.857591194 | 0.640621771 | APOA5; APOA5 | 37 | 11 |
| cg00521464 | 7.13E−10 | 3.03E−04 | −0.216964119 | 0.216964119 | loss | 0.696957155 | 0.479993035 | | 37 | 17 |
| cg08703522 | 3.52E−08 | 1.50E−02 | −0.216946596 | 0.216946596 | loss | 0.339944068 | 0.122997472 | | 37 | 17 |
| cg08052120 | 7.13E−10 | 3.03E−04 | −0.216944556 | 0.216944556 | loss | 0.634732126 | 0.417787571 | ZNF649 | 37 | 19 |
| cg12277789 | 7.77E−10 | 3.30E−04 | −0.216935002 | 0.216935002 | loss | 0.75422696 | 0.537291959 | | 37 | 10 |
| cg06860721 | 1.10E−09 | 4.65E−04 | −0.216932646 | 0.216932646 | loss | 0.646296387 | 0.429363741 | NCOA4; NCOA4; NCOA4 | 37 | 10 |
| cg21620139 | 1.68E−09 | 7.12E−04 | −0.216926908 | 0.216926908 | loss | 0.890234755 | 0.673307847 | C6orf25; C6orf25; C6orf25; | 37 | 6 |
| | | | | | | | | C6orf25; C6orf25; C6orf25 | | |
| cg05382501 | 7.13E−10 | 3.03E−04 | −0.216879074 | 0.216879074 | loss | 0.802022851 | 0.585143776 | | 37 | 4 |
| cg02555807 | 7.13E−10 | 3.03E−04 | −0.21687364 | 0.21687364 | loss | 0.736787075 | 0.519913435 | | 37 | 15 |
| cg16166011 | 1.19E−09 | 5.07E−04 | −0.216857705 | 0.216857705 | loss | 0.781068128 | 0.564210424 | ADD2; ADD2; ADD2; ADD2; | 37 | 2 |
| | | | | | | | | ADD2 | | |
| cg14338779 | 1.54E−09 | 6.54E−04 | −0.2168487 | 0.2168487 | loss | 0.833558342 | 0.616709641 | PTPRN2; PTPRN2; PTPRN2 | 37 | 7 |
| cg11193342 | 8.80E−09 | 3.74E−03 | −0.216803358 | 0.216803358 | loss | 0.437754317 | 0.220950959 | | 37 | 12 |
| cg22921760 | 7.13E−10 | 3.03E−04 | −0.216785757 | 0.216785757 | loss | 0.628342957 | 0.4115572 | SALL2; SALL2 | 37 | 14 |
| cg16591659 | 8.47E−10 | 3.60E−04 | −0.215782751 | 0.216782751 | loss | 0.778692157 | 0.561909406 | | 37 | 17 |
| cg02960125 | 7.13E−10 | 3.03E−04 | −0.216775864 | 0.216775864 | loss | 0.922211311 | 0.705435447 | ZNF648 | 37 | 1 |
| cg10012623 | 1.19E−09 | 5.07E−04 | −0.216758493 | 0.216758493 | loss | 0.88198134 | 0.665222847 | CHURC1-FNTB; CHURC1- | 37 | 14 |
| | | | | | | | | FNTB | | |
| cg04428163 | 7.13E−10 | 3.03E−04 | −0.216750476 | 0.216750476 | loss | 0.400963758 | 0.184213282 | SDCBP2; SDCBP2; SDCBP2; | 37 | 20 |
| | | | | | | | | FKBP1A-SDC8P2 | | |
| cg20115910 | 7.13E−10 | 3.03E−04 | −0.216744314 | 0.216744314 | loss | 0.866426502 | 0.649682188 | KCNH3 | 37 | 12 |
| cg01730666 | 7.13E−10 | 3.03E−04 | −0.216743586 | 0.216743586 | loss | 0.493831145 | 0.277087559 | CD300LG; CD300LG; CD300LG; | 37 | 17 |
| | | | | | | | | CD300LG | | |
| cg21100167 | 6.35E−09 | 2.70E−03 | −0.216743263 | 0.216743263 | loss | 0.599952881 | 0.383209618 | PCBP3; PCBP3 | 37 | 21 |
| cg02400942 | 7.13E−10 | 3.03E−04 | −0.216735824 | 0.216735824 | loss | 0.778358383 | 0.561622559 | CSPG4 | 37 | 15 |
| cg21325715 | 4.57E−09 | 1.94E−03 | −0.216734824 | 0.216734824 | loss | 0.632473442 | 0.415738618 | | 37 | 17 |
| cg06653052 | 1.83E−09 | 7.75E−04 | 0.216726911 | 0.216726911 | gain | 0.234165619 | 0.450892529 | C19orf24 | 37 | 19 |
| cg21012874 | 7.13E−10 | 3.03E−04 | −0.216720334 | 0.216720334 | loss | 0.522152292 | 0.305431959 | SNCG; MMRN2 | 37 | 10 |
| cg18947360 | 1.10E−09 | 4.65E−04 | −0.216710803 | 0.216710803 | loss | 0.409379979 | 0.192669176 | | 37 | 1 |
| cg08241939 | 7.13E−10 | 3.03E−04 | −0.216702103 | 0.216702103 | loss | 0.727393185 | 0.510691082 | | 37 | 1 |
| cg26254345 | 8.55E−08 | 3.63E−02 | −0.216684652 | 0.216684652 | loss | 0.86692907 | 0.650244418 | | 37 | 10 |
| cg12843033 | 7.77E−10 | 3.30E−04 | −0.216676022 | 0.216676022 | loss | 0.750194187 | 0.533518165 | AIRE | 37 | 21 |
| cg13903421 | 4.21E−09 | 1.79E−03 | −0.216660093 | 0.216660093 | loss | 0.410082981 | 0.193422888 | WNT6 | 37 | 2 |
| cg26428830 | 1.19E−09 | 5.07E−04 | −0.216620921 | 0.216620921 | loss | 0.695070804 | 0.478449882 | | 37 | 11 |
| cg21581504 | 7.13E−10 | 3.03E−04 | −0.216610365 | 0.216610365 | loss | 0.708574942 | 0.491964576 | CNRIP1; CNRIP1 | 37 | 2 |
| cg06556497 | 2.78E−09 | 1.18E−03 | −0.216594632 | 0.216594632 | loss | 0.501720579 | 0.285126047 | ATP9B | 37 | 18 |
| cg12696328 | 7.77E−10 | 3.30E−04 | −0.216593333 | 0.216593333 | loss | 0.364730342 | 0.148137009 | | 37 | 1 |
| cg15572235 | 6.30E−08 | 2.68E−02 | −0.216590934 | 0.216590934 | loss | 0.488207883 | 0.271616949 | ZNF890P; ZNF890P | 37 | 7 |
| cg26102082 | 7.13E−10 | 3.03E−04 | −0.216572998 | 0.216572998 | loss | 0.955279298 | 0.7387063 | NGFR; LOC100288866 | 37 | 17 |
| cg04757350 | 1.73E−09 | 7.34E−04 | −0.216556075 | 0.216556075 | loss | 0.841647238 | 0.625091163 | | 37 | 10 |
| cg09452027 | 7.13E−10 | 3.03E−04 | −0.216511972 | 0.216511972 | loss | 0.681836466 | 0.465324494 | SLC3SC1; SLC35C1; SLC35C1 | 37 | 11 |
| cg04188937 | 7.13E−10 | 3.03E−04 | −0.216504237 | 0.216504237 | loss | 0.705664702 | 0.489160465 | | 37 | 5 |
| cg08425070 | 1.73E−09 | 7.34E−04 | −0.216438158 | 0.216438158 | loss | 0.630293926 | 0.413855769 | SRRM3 | 37 | 7 |
| cg16359657 | 2.16E−09 | 9.17E−04 | −0.216431815 | 0.216431815 | loss | 0.522062721 | 0.305630906 | RIMS3 | 37 | 1 |
| cg02853387 | 1.83E−09 | 7.75E−04 | −0.2164063 | 0.2164063 | loss | 0.656609453 | 0.440203153 | DSCAML1 | 37 | 11 |
| cg14651210 | 3.67E−08 | 1.56E−02 | −0.216402535 | 0.216402535 | loss | 0.812664611 | 0.596262076 | | 37 | 1 |
| cg24724587 | 9.23E−10 | 3.92E−04 | −0.216397468 | 0.216397468 | loss | 0.571418457 | 0.355020986 | DDAH1; DDAH1 | 37 | 1 |
| cg26544377 | 9.23E−10 | 3.92E−04 | −0.216385214 | 0.216385214 | loss | 0.794805491 | 0.578421276 | | 37 | 7 |
| cg12720965 | 8.80E−09 | 3.74E−03 | −0.216383822 | 0.216383822 | loss | 0.780277381 | 0.563893559 | KIF26B | 37 | 1 |
| cg17806162 | 7.77E−10 | 3.30E−04 | −0.216373362 | 0.216373362 | loss | 0.408786221 | 0.192412859 | | 37 | 1 |
| cg03287299 | 3.28E−09 | 1.39E−03 | −0.216372292 | 0.216372292 | loss | 0.339457292 | 0.123085 | LINC00654; LINC00654 | 37 | 20 |
| cg14578009 | 9.94E−08 | 4.22E−02 | −0.216335493 | 0.216335493 | loss | 0.473342183 | 0.25700669 | | 37 | 5 |
| cg23849826 | 5.39E−09 | 2.29E−03 | −0.215334135 | 0.216334135 | loss | 0.811279947 | 0.594945812 | THBS1 | 37 | 15 |
| cg00225006 | 9.23E−10 | 3.92E−04 | −0.216300375 | 0.216300375 | loss | 0.880776398 | 0.664476024 | WHAMM | 37 | 15 |
| cg04850045 | 1.42E−09 | 6.01E−04 | −0.216294705 | 0.216294705 | loss | 0.766762434 | 0.550467729 | | 37 | 2 |
| cg03921696 | 7.77E−10 | 3.30E−04 | −0.216216106 | 0.216216106 | loss | 0.603143236 | 0.386927129 | | 37 | 3 |
| cg05662444 | 5.41E−08 | 2.30E−02 | −0.216201984 | 0.216201984 | loss | 0.350741251 | 0.134539266 | | 37 | 5 |
| cg20957370 | 8.80E−09 | 3.74E−03 | −0.216189866 | 0.216189866 | loss | 0.694898772 | 0.478708905 | | 37 | 5 |
| cg25529304 | 7.13E−10 | 3.03E−04 | −0.216186166 | 0.216186166 | loss | 0.71340676 | 0.497220594 | | 37 | 5 |
| cg18290190 | 1.16E−07 | 4.91E−02 | −0.216181473 | 0.216181473 | loss | 0.872006043 | 0.655824571 | RPH3AL; RPH3AL; RPH3AL; | 37 | 17 |
| | | | | | | | | RPH3AL | | |
| cg17484472 | 1.01E−09 | 4.27E−04 | −0.216170971 | 0.216170971 | loss | 0.573268689 | 0.357097718 | | 37 | 16 |
| cg00516481 | 7.77E−10 | 3.30E−04 | −0.216169083 | 0.216169083 | loss | 0.44220163 | 0.226032547 | PDE9A; PDE9A; PDE9A; PDE9A; | 37 | 21 |
| | | | | | | | | PDE9A; PDE9A; PDE9A; | | |
| | | | | | | | | PDE9A; PDE9A; PDE9A; PDE9A; | | |
| | | | | | | | | PDE9A; PDE9A; PDE9A; | | |
| | | | | | | | | PDE9A; PDE9A; PDE9A; | | |
| | | | | | | | | PDE9A; PDE9A; PDE9A | | |
| cg03237218 | 7.13E−10 | 3.03E−04 | −0.216159036 | 0.216159036 | loss | 0.74203113 | 0.525872094 | SLFN12 | 37 | 17 |
| cg04369651 | 7.13E−10 | 3.03E−04 | −0.216135577 | 0.216135577 | loss | 0.826728794 | 0.610593218 | MYT1L | 37 | 2 |
| cg19820919 | 1.02E−09 | 4.33E−04 | −0.216134623 | 0.216134623 | loss | 0.468873612 | 0.252738988 | SPSB1 | 37 | 1 |
| cg19135273 | 7.13E−10 | 3.03E−04 | −0.216129375 | 0.216129375 | loss | 0.681563298 | 0.465433924 | MROH5; MROH5; MROH5 | 37 | 8 |
| cg16148593 | 3.57E−09 | 1.52E−03 | −0.216103943 | 0.216103943 | loss | 0.362991875 | 0.146887932 | PHYHIP; PHYHIP | 37 | 8 |
| cg24194775 | 2.69E−08 | 1.14E−02 | −0.215085703 | 0.216085703 | loss | 0.567188515 | 0.351102812 | NPR2 | 37 | 9 |
| cg08277369 | 3.28E−09 | 1.39E−03 | −0.216082512 | 0.216082512 | loss | 0.35725873 | 0.141176218 | TLDC1 | 37 | 16 |
| cg08578641 | 7.13E−10 | 3.03E−04 | −0.216072895 | 0.216072895 | loss | 0.888772036 | 0.672699141 | FAM219A; FAM219A; FAM219A; | 37 | 9 |
| | | | | | | | | FAM219A; FAM219A; | | |
| | | | | | | | | DNAI1; DNAI1; FAM219A | | |
| cg00669347 | 8.47E−10 | 3.60E−04 | −0.216066132 | 0.216066132 | loss | 0.639377474 | 0.423311341 | | 37 | 16 |
| cg01571001 | 1.12E−08 | 4.76E−03 | −0.216030803 | 0.216030803 | loss | 0.846460668 | 0.630429865 | | 37 | 3 |
| cg19808847 | 1.81E−08 | 7.69E−03 | −0.216026189 | 0.216026189 | loss | 0.760012783 | 0.543986594 | | 37 | 8 |
| cg19299755 | 3.88E−09 | 1.65E−03 | −0.216024806 | 0.216024806 | loss | 0.496745536 | 0.280720729 | | 37 | 11 |
| cg25965498 | 9.94E−08 | 4.22E−02 | −0.216022528 | 0.216022528 | loss | 0.786340281 | 0.570317753 | VGLL4; VGLL4 | 37 | 3 |
| cg13506559 | 7.13E−10 | 3.03E−04 | −0.216004314 | 0.216004314 | loss | 0.831032902 | 0.615028588 | | 37 | 9 |
| cg18981477 | 8.47E−10 | 3.60E−04 | −0.216004209 | 0.216004209 | loss | 0.629406626 | 0.413402418 | | 37 | 20 |
| cg06318402 | 8.56E−10 | 3.63E−04 | −0.215981583 | 0.215981583 | loss | 0.654939348 | 0.438957765 | | 37 | 1 |
| cg16301924 | 7.13E−10 | 3.03E−04 | −0.215945666 | 0.215945666 | loss | 0.825475477 | 0.609529812 | LECT1; LECT1 | 37 | 13 |
| cg18972998 | 7.13E−10 | 3.03E−04 | −0.215935865 | 0.215935865 | loss | 0.846107883 | 0.630172018 | TTC22; TTC22 | 37 | 1 |
| cg19022717 | 7.13E−10 | 3.03E−04 | −0.215932757 | 0.215932757 | loss | 0.804829451 | 0.588896694 | LRRC71 | 37 | 1 |
| cg11755201 | 9.23E−10 | 3.92E−04 | −0.215888065 | 0.215888065 | loss | 0.737281647 | 0.521393582 | CAMTA1 | 37 | 1 |
| cg05038469 | 1.99E−09 | 8.43E−04 | −0.215875865 | 0.215875865 | loss | 0.528952647 | 0.313076782 | | 37 | 14 |
| cg04499083 | 1.54E−09 | 6.54E−04 | −0.215869454 | 0.215869454 | loss | 0.794003672 | 0.578134218 | | 37 | 1 |
| cg16699148 | 4.29E−08 | 1.82E−02 | −0.215844806 | 0.215844806 | loss | 0.448192623 | 0.232347817 | TACSTD2; TACSTD2 | 37 | 1 |
| cg24789467 | 7.13E−10 | 3.03E−04 | −0.215840218 | 0.215840218 | loss | 0.297276443 | 0.081436225 | SHROOM1; SHROOM1 | 37 | 5 |
| cg25336198 | 7.77E−10 | 3.30E−04 | −0.215831194 | 0.215831194 | loss | 0.607719058 | 0.391887865 | INS; INS; INS-IGF2; INS- | 37 | 11 |
| | | | | | | | | IGF2; INS; INS; INS; INS; INS- | | |
| | | | | | | | | IGF2; INS-IGF2 | | |
| cg05106191 | 7.13E−10 | 3.03E−04 | −0.215827763 | 0.215827763 | loss | 0.819942634 | 0.604114871 | PAX8; PAX8; PAX8; PAX8; PAX8; | 37 | 2 |
| | | | | | | | | PAX8; PAX8; PAX8 | | |
| cg21903646 | 2.56E−09 | 1.09E−03 | −0.215821461 | 0.215821461 | loss | 0.351651804 | 0.135830343 | | 37 | 6 |
| cg02574464 | 1.83E−09 | 7.75E−04 | −0.215783075 | 0.215783075 | loss | 0.423702728 | 0.207919653 | CNGB1; CNG81 | 37 | 16 |
| cg18730174 | 3.57E−09 | 1.52E−03 | −0.215777757 | 0.215777757 | loss | 0.834953387 | 0.619175629 | MIR100HG | 37 | 11 |
| cg25633271 | 7.13E−10 | 3.03E−04 | −0.215761391 | 0.215761391 | loss | 0.880244768 | 0.664483376 | SRCIN1 | 37 | 17 |
| cg14383174 | 2.78E−09 | 1.18E−03 | −0.215734417 | 0.215734417 | loss | 0.783664164 | 0.567929747 | | 37 | 17 |
| cg00270259 | 7.13E−10 | 3.03E−04 | −0.215730846 | 0.215730846 | loss | 0.737741981 | 0.522011135 | SFTPC; SFTPC; SFTPC | 37 | 8 |
| cg18774154 | 6.35E−09 | 2.70E−03 | −0.215728242 | 0.215728242 | loss | 0.817076142 | 0.6013479 | | 37 | 2 |
| cg17108141 | 8.47E−10 | 3.60E−04 | −0.215721529 | 0.215721529 | loss | 0.556721158 | 0.340999629 | DDAH1; DDAH1 | 37 | 1 |
| cg07477928 | 1.92E−08 | 8.15E−03 | −0.215701033 | 0.215701033 | loss | 0.791052427 | 0.575351394 | SVEP1 | 37 | 9 |
| cg02668773 | 1.01E−09 | 4.27E−04 | −0.215699494 | 0.215699494 | loss | 0.508479358 | 0.292779865 | MPO | 37 | 17 |
| cg01812146 | 1.01E−09 | 4.27E−04 | −0.2156879 | 0.2156879 | loss | 0.600370911 | 0.384683012 | ARHGAP29 | 37 | 1 |
| cg04001333 | 7.48E−09 | 3.18E−03 | −0.215583684 | 0.215583684 | loss | 0.558368813 | 0.342785129 | FLVCR2 | 37 | 14 |
| cg08746853 | 2.35E−09 | 9.98E−04 | −0.215577178 | 0.215577178 | loss | 0.462811872 | 0.247234694 | HIST4H4 | 37 | 12 |
| cg00548822 | 7.13E−10 | 3.03E−04 | −0.21555338 | 0.21555338 | loss | 0.908655468 | 0.693102088 | | 37 | 11 |
| cg18337938 | 7.13E−10 | 3.03E−04 | −0.21553391 | 0.21553391 | loss | 0.473284992 | 0.257751082 | | 37 | 1 |
| cg20581874 | 3.02E−09 | 1.28E−03 | −0.215526619 | 0.215526619 | loss | 0.502154725 | 0.286628106 | | 37 | 14 |
| cg17624536 | 9.34E−10 | 3.97E−04 | −0.215512785 | 0.215512785 | loss | 0.437172479 | 0.221659694 | | 37 | 6 |
| cg00026362 | 7.13E−10 | 3.03E−04 | −0.215497779 | 0.215497779 | loss | 0.570598874 | 0.355101094 | LOC100132354 | 37 | 6 |
| cg03647927 | 7.13E−10 | 3.03E−04 | −0.21548559 | 0.21548559 | loss | 0.712777396 | 0.497291806 | ARHGAP23 | 37 | 17 |
| cg09613495 | 7.13E−10 | 3.03E−04 | −0.215480828 | 0.215480828 | loss | 0.43104734 | 0.215566512 | GUCA1A | 37 | 6 |
| cg11953516 | 1.10E−09 | 4.65E−04 | −0.215477691 | 0.215477691 | loss | 0.379311636 | 0.163833945 | GCKR; FNDC4 | 37 | 2 |
| cg25818214 | 7.13E−10 | 3.03E−04 | −0.215465852 | 0.215465852 | loss | 0.548397481 | 0.332931629 | PRICKLE2; PRICKLE2 | 37 | 3 |
| cg15679892 | 1.03E−08 | 4.39E−03 | −0.215440783 | 0.215440783 | loss | 0.662875289 | 0.447434506 | | 37 | 7 |
| cg08133584 | 7.13E−10 | 3.03E−04 | −0.215428533 | 0.215428533 | loss | 0.515103862 | 0.299675329 | C16orf11 | 37 | 16 |
| cg03610228 | 7.13E−10 | 3.03E−04 | −0.215385558 | 0.215385558 | loss | 0.312547847 | 0.097162289 | | 37 | 10 |
| cg10130564 | 6.81E−08 | 2.89E−02 | −0.215374598 | 0.215374598 | loss | 0.744206287 | 0.528831688 | TAGLN; TAGLN; LOC100652768 | 37 | 11 |
| cg13026730 | 3.67E−08 | 1.56E−02 | −0.215338447 | 0.215338447 | loss | 0.390081396 | 0.174742949 | EIF4E3; EIF4E3; GPR27; EIF4E3 | 37 | 3 |
| cg15280358 | 2.56E−09 | 1.09E−03 | −0.21533774 | 0.21533774 | loss | 0.76333457 | 0.547996829 | ADAM12; ADAM12 | 37 | 10 |
| cg09284655 | 1.54E−09 | 6.54E−04 | −0.215308251 | 0.215308251 | loss | 0.790354751 | 0.5750465 | | 37 | 14 |
| cg15078822 | 1.42E−09 | 6.01E−04 | −0.21527892 | 0.21527892 | loss | 0.685175343 | 0.469896424 | | 37 | 7 |
| cg01093878 | 7.84E−10 | 3.33E−04 | −0.215269643 | 0.215269643 | loss | 0.56468319 | 0.349413547 | AMER3; AMER3; AMER3; AMER3 | 37 | 2 |
| cg23139982 | 8.55E−08 | 3.63E−02 | −0.215263973 | 0.215263973 | loss | 0.563441796 | 0.348177824 | | 37 | 4 |
| cg22369879 | 5.84E−08 | 2.48E−02 | −0.215250253 | 0.215250253 | loss | 0.767320406 | 0.552070153 | | 37 | 3 |
| cg17665699 | 9.23E−10 | 3.92E−04 | −0.215247958 | 0.215247958 | loss | 0.800593317 | 0.585345359 | ULK2; ULK2 | 37 | 17 |
| cg11410859 | 1.01E−09 | 4.27E−04 | −0.215224407 | 0.215224407 | loss | 0.84958556 | 0.634361153 | SCN5A; SCN5A; SCN5A; SCN5A; | 37 | 3 |
| | | | | | | | | SCN5A; SCN5A | | |
| cg01089602 | 7.13E−10 | 3.03E−04 | −0.215137325 | 0.215137325 | loss | 0.761397925 | 0.5462606 | F7; F7; F7; F7 | 37 | 13 |
| cg10591174 | 7.13E−10 | 3.03E−04 | −0.215122719 | 0.215122719 | loss | 0.501884077 | 0.286761359 | MYL2; MYL2 | 37 | 12 |
| cg03272408 | 7.13E−10 | 3.03E−04 | −0.215115342 | 0.215115342 | loss | 0.709237083 | 0.494121741 | COPZ2 | 37 | 17 |
| cg07033202 | 7.13E−10 | 3.03E−04 | −0.215108026 | 0.215108026 | loss | 0.367826238 | 0.152718212 | | 37 | 3 |
| cg09386265 | 7.13E−10 | 3.03E−04 | −0.21509967 | 0.21509967 | loss | 0.804829906 | 0.589730235 | | 37 | 19 |
| cg06080250 | 7.13E−10 | 3.03E−04 | −0.215097888 | 0.215097888 | loss | 0.839599947 | 0.624502059 | | 37 | 4 |
| cg18208742 | 9.23E−10 | 3.92E−04 | −0.215092694 | 0.215092694 | loss | 0.8787232 | 0.663630506 | GFAP; GFAP; GFAP; GFAP; GFAP; | 37 | 17 |
| | | | | | | | | GFAP | | |
| cg16419906 | 7.13E−10 | 3.03E−04 | −0.215046169 | 0.215046169 | loss | 0.775776151 | 0.560729982 | SHROOM1 | 37 | 5 |
| cg13941021 | 7.13E−10 | 3.03E−04 | −0.215039068 | 0.215039068 | loss | 0.304110175 | 0.089071107 | PPP1CC; PPP1CC | 37 | 12 |
| cg25507767 | 7.13E−10 | 3.03E−04 | −0.214992403 | 0.214992403 | loss | 0.829106326 | 0.614113924 | TMEM213; ATP6V0A4; ATP6V0A4 | 37 | 7 |
| cg14652095 | 7.13E−10 | 3.03E−04 | −0.214958502 | 0.214958502 | loss | 0.613644396 | 0.398685894 | HIST1H1A; HIST1H1A | 37 | 6 |
| cg23989110 | 1.19E−09 | 5.07E−04 | −0.214932299 | 0.214932299 | loss | 0.728688787 | 0.513756488 | MAP3K6 | 37 | 1 |
| cg19481355 | 1.42E−09 | 6.01E−04 | −0.214930143 | 0.214930143 | loss | 0.906917996 | 0.691987853 | LOC401242 | 37 | 6 |
| cg12523932 | 2.35E−09 | 9.98E−04 | −0.214926012 | 0.214926012 | loss | 0.282719972 | 0.06779396 | KLB | 37 | 4 |
| cg27008631 | 7.13E−10 | 3.03E−04 | −0.214917291 | 0.214917291 | loss | 0.668572085 | 0.453654794 | NTN5; NTN5; SEC1P | 37 | 19 |
| cg11204974 | 3.57E−09 | 1.52E−03 | −0.21491577 | 0.21491577 | loss | 0.547695417 | 0.332779647 | | 37 | 14 |
| cg25417766 | 1.73E−09 | 7.34E−04 | −0.214892636 | 0.214892636 | loss | 0.376550936 | 0.1616583 | | 37 | 19 |
| cg20935489 | 2.78E−09 | 1.18E−03 | −0.214887243 | 0.214887243 | loss | 0.655549843 | 0.4416626 | | 37 | 8 |
| cg00152942 | 7.13E−10 | 3.03E−04 | −0.214855561 | 0.214855561 | loss | 0.663727443 | 0.448871882 | CPNE2 | 37 | 16 |
| cg15799267 | 7.77E−10 | 3.30E−04 | −0.214846682 | 0.214846682 | loss | 0.427819011 | 0.212972329 | ALOX15B; ALOX15B; ALOX15B | 37 | 17 |
| cg24864576 | 3.28E−09 | 1.39E−03 | −0.214844024 | 0.214844024 | loss | 0.513962632 | 0.299118608 | | 37 | 18 |
| cg23666491 | 7.13E−10 | 3.03E−04 | −0.214773853 | 0.214773853 | loss | 0.823037705 | 0.608263853 | | 37 | 6 |
| cg23771366 | 7.84E−10 | 3.33E−04 | −0.214756623 | 0.214756623 | loss | 0.462809723 | 0.2480531 | PRSS23 | 37 | 11 |
| cg18509185 | 5.41E−08 | 2.30E−02 | −0.21475339 | 0.21475339 | loss | 0.748934896 | 0.534181506 | SOBP | 37 | 6 |
| cg25464265 | 7.48E−09 | 3.18E−03 | −0.214752949 | 0.214752949 | loss | 0.846098891 | 0.631345941 | | 37 | 1 |
| cg16743289 | 7.77E−10 | 3.30E−04 | −0.214744304 | 0.214744304 | loss | 0.52954134 | 0.314797035 | CACYBP; CACYBP | 37 | 1 |
| cg12243267 | 1.42E−09 | 6.01E−04 | −0.214727257 | 0.214727257 | loss | 0.767497304 | 0.552770047 | USP29 | 37 | 19 |
| cg12391643 | 9.54E−09 | 4.05E−03 | −0.214723873 | 0.214723873 | loss | 0.833788826 | 0.619064953 | | 37 | 2 |
| cg12317414 | 7.13E−10 | 3.03E−04 | −0.214698209 | 0.214698209 | loss | 0.329809115 | 0.115110906 | SYNPO2L; SYNPO2L | 37 | 10 |
| cg03632245 | 7.13E−10 | 3.03E−04 | −0.214644402 | 0.214644402 | loss | 0.792306538 | 0.577662135 | | 37 | 5 |
| cg25556008 | 7.13E−10 | 3.03E−04 | −0.214615592 | 0.214615592 | loss | 0.905935315 | 0.691319724 | GNG7 | 37 | 19 |
| cg13587180 | 6.89E−09 | 2.93E−03 | −0.214887881 | 0.214587881 | loss | 0.721656175 | 0.507068294 | SHISA3 | 37 | 4 |
| cg16750777 | 9.23E−10 | 3.92E−04 | −0.214581518 | 0.214581518 | loss | 0.736756894 | 0.522175376 | NKD1 | 37 | 16 |
| cg13072940 | 7.13E−10 | 3.03E−04 | −0.214573307 | 0.214573307 | loss | 0.42160123 | 0.207027924 | MON1A; MON1A; MON1A; | 37 | 3 |
| | | | | | | | | MON1A | | |
| cg20902757 | 7.13E−10 | 3.03E−04 | −0.214572496 | 0.214572496 | loss | 0.648138843 | 0.433566347 | | 37 | 8 |
| cg13945148 | 9.23E−10 | 3.92E−04 | −0.214522267 | 0.214522267 | loss | 0.595152296 | 0.380630029 | | 37 | 10 |
| cg11941060 | 7.77E−10 | 3.30E−04 | −0.214499133 | 0.214499133 | loss | 0.880805404 | 0.666306271 | SRPRB | 37 | 3 |
| cg01794156 | 9.22E−08 | 3.91E−02 | −0.214477532 | 0.214477532 | loss | 0.593691626 | 0.379214094 | PKP3 | 37 | 11 |
| cg14150727 | 7.13E−10 | 3.03E−04 | −0.214446954 | 0.214446954 | loss | 0.762523872 | 0.548076918 | LGR6; LGR6; LGR6 | 37 | 1 |
| cg23337866 | 7.77E−10 | 3.30E−04 | −0.214418782 | 0.214418782 | loss | 0.536982917 | 0.322564135 | | 37 | 4 |
| cg22481770 | 7.13E−10 | 3.03E−04 | −0.214417207 | 0.214417207 | loss | 0.346502681 | 0.132085474 | | 37 | 3 |
| cg07141002 | 9.23E−10 | 3.92E−04 | −0.214399585 | 0.214399585 | loss | 0.427860485 | 0.2134609 | H1F0 | 37 | 22 |
| cg20793420 | 7.13E−10 | 3.03E−04 | −0.21437391 | 0.21437391 | loss | 0.359335951 | 0.144962041 | | 37 | 22 |
| cg09487101 | 7.77E−10 | 3.30E−04 | −0.214369743 | 0.214369743 | loss | 0.533744566 | 0.319374824 | EFNA2 | 37 | 19 |
| cg08535474 | 4.97E−09 | 2.11E−03 | −0.214293071 | 0.214293071 | loss | 0.70039103 | 0.486097959 | | 37 | 17 |
| cg15410675 | 4.21E−09 | 1.79E−03 | −0.214279716 | 0.214279716 | loss | 0.522296951 | 0.308017235 | TRIM2; TRIM2 | 37 | 4 |
| cg05357695 | 5.85E−09 | 2.49E−03 | −0.214270553 | 0.214270553 | loss | 0.785867889 | 0.571597335 | SLC7A8; SLC7A8; SLC7A8; SLC7A8; | 37 | 14 |
| | | | | | | | | SLC7A8; SLC7A8; SLC7A8 | | |
| cg13459606 | 9.23E−10 | 3.92E−04 | −0.214241927 | 0.214241927 | loss | 0.853875315 | 0.639533388 | GRB10 | 37 | 7 |
| cg23044903 | 7.13E−10 | 3.03E−04 | −0.214235455 | 0.214235455 | loss | 0.760589308 | 0.546353853 | CPLX3 | 37 | 15 |
| cg08906015 | 7.77E−10 | 3.30E−04 | −0.214220021 | 0.214220021 | loss | 0.898136715 | 0.683916694 | DACT3 | 37 | 19 |
| cg13868361 | 1.10E−09 | 4.65E−04 | −0.214217072 | 0.214217072 | loss | 0.661704113 | 0.447487041 | GTF2B | 37 | 1 |
| cg05180887 | 1.92E−08 | 8.15E−03 | −0.214215221 | 0.214215221 | loss | 0.627362004 | 0.413145782 | MYT1L | 37 | 2 |
| cg20475486 | 9.23E−10 | 3.92E−04 | −0.214214821 | 0.214214821 | loss | 0.820019515 | 0.605804694 | | 37 | 14 |
| cg21147203 | 4.64E−08 | 1.97E−02 | −0.214189907 | 0.214189907 | loss | 0.695012672 | 0.480822765 | C5orf66 | 37 | 5 |
| cg05275595 | 2.12E−08 | 9.01E−03 | −0.214179208 | 0.214179208 | loss | 0.552089196 | 0.337909988 | CD177 | 37 | 19 |
| cg04268624 | 1.10E−09 | 4.65E−04 | −0.214173703 | 0.214173703 | loss | 0.805608762 | 0.591435059 | | 37 | 5 |
| cg09469223 | 1.09E−08 | 4.62E−03 | −0.214173024 | 0.214173024 | loss | 0.692684971 | 0.478511947 | SBK2 | 37 | 19 |
| cg00942918 | 2.78E−09 | 1.18E−03 | −0.214155478 | 0.214155478 | loss | 0.542901266 | 0.328745788 | HIST1H3A; HIST1H4A | 37 | 6 |
| cg26112661 | 7.13E−10 | 3.03E−04 | −0.214135276 | 0.214135276 | loss | 0.426484058 | 0.212348782 | MAGOH; LOC100507564 | 37 | 1 |
| cg06061238 | 7.77E−10 | 3.30E−04 | −0.214128396 | 0.214128396 | loss | 0.750631213 | 0.536502818 | GPR123 | 37 | 10 |
| cg08282428 | 6.89E−09 | 2.93E−03 | −0.214105804 | 0.214105804 | loss | 0.62954234 | 0.415436535 | RBM46; RBM46; RBM46 | 37 | 4 |
| cg24382070 | 7.13E−10 | 3.03E−04 | −0.214103789 | 0.214103789 | loss | 0.432226589 | 0.2181228 | LGI3 | 37 | 8 |
| cg00141308 | 1.35E−08 | 5.74E−03 | −0.214074488 | 0.214074488 | loss | 0.5412899 | 0.327215412 | | 37 | 22 |
| cg17884383 | 7.13E−10 | 3.03E−04 | −0.214070466 | 0.214070466 | loss | 0.652908408 | 0.438837941 | KLC3 | 37 | 19 |
| cg23188684 | 1.16E−07 | 4.91E−02 | −0.214056826 | 0.214056826 | loss | 0.470006272 | 0.255949445 | DOC2GP | 37 | 11 |
| cg03841312 | 9.23E−10 | 3.92E−04 | −0.214053484 | 0.214053484 | loss | 0.886597996 | 0.672544512 | MIRLET7BHG; MIRLET7A3; | 37 | 22 |
| | | | | | | | | MIRLET7B; MIR4763 | | |
| cg15254238 | 7.13E−10 | 3.03E−04 | −0.214047355 | 0.214047355 | loss | 0.337061796 | 0.123014441 | TET1 | 37 | 10 |
| cg04737087 | 7.13E−10 | 3.03E−04 | −0.214042517 | 0.214042517 | loss | 0.390725817 | 0.1766833 | ANKRD53; ANKRD53 | 37 | 2 |
| cg08756033 | 2.16E−09 | 9.17E−04 | −0.214027692 | 0.214027692 | loss | 0.729743909 | 0.515716218 | MEDAG; TEX26-A51 | 37 | 13 |
| cg03666300 | 7.13E−10 | 3.03E−04 | −0.214024138 | 0.214024138 | loss | 0.892683738 | 0.6786596 | | 37 | 16 |
| cg16101008 | 2.69E−08 | 1.14E−02 | −0.214021842 | 0.214021842 | loss | 0.342422204 | 0.128400362 | CASZ1 | 37 | 1 |
| cg22674497 | 1.30E−09 | 5.52E−04 | −0.214013119 | 0.214013119 | loss | 0.65454756 | 0.440534441 | USH1G; USH1G | 37 | 17 |
| cg01189038 | 7.13E−10 | 3.03E−04 | −0.213980566 | 0.213980566 | loss | 0.650794577 | 0.436814012 | ECE2; ECE2; ECE2; ECE2 | 37 | 3 |
| cg13581527 | 7.13E−10 | 3.03E−04 | −0.213963702 | 0.213963702 | loss | 0.279897338 | 0.065933636 | | 37 | 7 |
| cg27401562 | 7.92E−08 | 3.36E−02 | −0.21395596 | 0.21395596 | loss | 0.52715213 | 0.313196171 | | 37 | X |
| cg19478105 | 7.13E−10 | 3.03E−04 | −0.213943447 | 0.213943447 | loss | 0.820143664 | 0.606200218 | SLIT1; ARHGAP19-SLIT1 | 37 | 10 |
| cg02343628 | 1.96E−08 | 8.33E−03 | 0.213943059 | 0.213943059 | gain | 0.528333664 | 0.742276724 | | 37 | 15 |
| cg15102365 | 1.01E−09 | 4.27E−04 | −0.213940264 | 0.213940264 | loss | 0.886108475 | 0.672168212 | GEMIN6 | 37 | 2 |
| cg10564498 | 8.47E−10 | 3.60E−04 | −0.213921977 | 0.213921977 | loss | 0.32556204 | 0.111640062 | AXL; AXL | 37 | 19 |
| cg24235633 | 7.13E−10 | 3.03E−04 | −0.213886754 | 0.213886754 | loss | 0.920837872 | 0.706951118 | CDIPT; CDIPT; CDIPT; CDIPT- | 37 | 16 |
| | | | | | | | | AS1; CDIPT-AS1 | | |
| cg08360599 | 7.13E−10 | 3.03E−04 | −0.21385342 | 0.21385342 | loss | 0.548974643 | 0.335121224 | ZBTB38 | 37 | 3 |
| cg09214412 | 8.80E−09 | 3.74E−03 | −0.213832711 | 0.213832711 | loss | 0.752694311 | 0.5388616 | RGMA; RGMA; RGMA; RGMA; | 37 | 15 |
| | | | | | | | | RGMA; RGMA | | |
| cg00335715 | 7.13E−10 | 3.03E−04 | −0.213812321 | 0.213812321 | loss | 0.269680217 | 0.055867896 | | 37 | 5 |
| cg17062938 | 7.13E−10 | 3.03E−04 | −0.213796579 | 0.213796579 | loss | 0.372884438 | 0.159087859 | | 37 | 7 |
| cg00520135 | 2.30E−08 | 9.75E−03 | −0.213795998 | 0.213795998 | loss | 0.721331609 | 0.507535612 | TPM1; TPM1; TPM1; TPM1; | 37 | 15 |
| | | | | | | | | TPM1; TPM1 | | |
| cg16246169 | 8.47E−10 | 3.60E−04 | −0.213772163 | 0.213772163 | loss | 0.671324992 | 0.457552829 | SOX2-OT; SOX2-OT; SOX2- | 37 | 3 |
| | | | | | | | | OT; SOX2-OT; SOX2- | | |
| | | | | | | | | OT; SOX2-OT | | |
| cg19811683 | 6.35E−09 | 2.70E−03 | −0.213757185 | 0.213757185 | loss | 0.739163279 | 0.525406094 | | 37 | 17 |
| cg26060667 | 1.19E−09 | 5.07E−04 | −0.213731266 | 0.213731266 | loss | 0.602523325 | 0.388792059 | GCSAML; GCSAML; GCSAML; | 37 | 1 |
| | | | | | | | | GCSAML; GCSAML; GCSAML | | |
| cg17057719 | 7.77E−10 | 3.30E−04 | −0.213724014 | 0.213724014 | loss | 0.444712932 | 0.230988918 | | 37 | 3 |
| cg25821245 | 7.13E−10 | 3.03E−04 | −0.21370655 | 0.21370655 | loss | 0.421679285 | 0.207972735 | LRRC2; LRRC2 | 37 | 3 |
| cg19158754 | 7.13E−10 | 3.03E−04 | −0.213676247 | 0.213676247 | loss | 0.519802953 | 0.306126706 | | 37 | 11 |
| cg19001789 | 9.23E−10 | 3.92E−04 | −0.213670546 | 0.213670546 | loss | 0.324693868 | 0.111023322 | | 37 | 13 |
| cg22760338 | 2.91E−08 | 1.23E−02 | −0.213635624 | 0.213635624 | loss | 0.563458777 | 0.349823153 | PRLR; PRLR; PRLR; PRLR; PRLR; | 37 | 5 |
| | | | | | | | | PRLR; PRLR | | |
| cg04753078 | 1.01E−09 | 4.27E−04 | −0.213627773 | 0.213627773 | loss | 0.424282732 | 0.210654959 | TNXB | 37 | 6 |
| cg00507679 | 7.84E−10 | 3.33E−04 | −0.213595415 | 0.213595415 | loss | 0.865368756 | 0.651773341 | TTBK1 | 37 | 6 |
| cg12499872 | 6.80E−08 | 2.89E−02 | −0.213593423 | 0.213593423 | loss | 0.777359911 | 0.563766488 | TEPP; TEPP | 37 | 16 |
| cg19522930 | 1.19E−09 | 5.07E−04 | −0.213593154 | 0.213593154 | loss | 0.79127636 | 0.577683206 | | 37 | 11 |
| cg21843594 | 1.10E−09 | 4.65E−04 | −0.213584954 | 0.213584954 | loss | 0.385618636 | 0.172033682 | HORMAD2 | 37 | 22 |
| cg09508938 | 4.29E−08 | 1.82E−02 | −0.213573636 | 0.213573636 | loss | 0.874679272 | 0.661105635 | | 37 | 6 |
| cg16058797 | 7.13E−10 | 3.03E−04 | −0.213562857 | 0.213562857 | loss | 0.262575226 | 0.049012369 | | 37 | 8 |
| cg09175009 | 7.13E−10 | 3.03E−04 | −0.213554185 | 0.213554185 | loss | 0.570174621 | 0.356620435 | TVP23A; TVP23A | 37 | 16 |
| cg05940452 | 4.49E−09 | 1.91E−03 | −0.213506952 | 0.213506952 | loss | 0.514627258 | 0.301120306 | FOXR1 | 37 | 11 |
| cg14485744 | 1.22E−08 | 5.16E−03 | −0.213502387 | 0.213502387 | loss | 0.851480428 | 0.637978041 | EXT1 | 37 | 8 |
| cg18517222 | 1.83E−09 | 7.75E−04 | −0.213485903 | 0.213485903 | loss | 0.722643032 | 0.509157129 | | 37 | 11 |
| cg10585661 | 1.42E−09 | 6.01E−04 | −0.213485321 | 0.213485321 | loss | 0.530048421 | 0.3165631 | FAM131A; FAM131A | 37 | 3 |
| cg15939523 | 4.64E−08 | 1.97E−02 | −0.21346911 | 0.21346911 | loss | 0.846385804 | 0.632916594 | | 37 | 4 |
| cg24750308 | 2.91E−08 | 1.23E−02 | −0.213464156 | 0.213464156 | loss | 0.502035026 | 0.288570871 | NOX4; NOX4; NOX4; NOX4 | 37 | 11 |
| cg11952839 | 7.13E−10 | 3.03E−04 | −0.213456519 | 0.213456519 | loss | 0.626352913 | 0.412896394 | NKX6-3; NKX6-3 | 37 | 8 |
| cg04629204 | 7.13E−10 | 3.03E−04 | −0.213452302 | 0.213452302 | loss | 0.78796046 | 0.574508159 | EXTL1 | 37 | 1 |
| cg02269643 | 2.35E−09 | 9.98E−04 | −0.213439285 | 0.213439285 | loss | 0.804505043 | 0.591065759 | | 37 | 14 |
| cg20391126 | 1.54E−09 | 6.54E−04 | −0.213432269 | 0.213432269 | loss | 0.698841634 | 0.485409365 | | 37 | 7 |
| cg09755872 | 8.47E−10 | 3.60E−04 | −0.213432003 | 0.213432003 | loss | 0.34605187 | 0.132619866 | | 37 | 7 |
| cg17588350 | 7.77E−10 | 3.30E−04 | −0.213417397 | 0.213417397 | loss | 0.938691921 | 0.725274524 | LINC00301 | 37 | 11 |
| cg13448197 | 5.85E−09 | 2.49E−03 | −0.21340039 | 0.21340039 | loss | 0.267328413 | 0.053928023 | | 37 | 10 |
| cg03738669 | 7.13E−10 | 3.03E−04 | −0.213375226 | 0.213375226 | loss | 0.752710749 | 0.539335524 | SLC4A11; SLC4A11; SLC4A11; | 37 | 20 |
| | | | | | | | | SLC4A11 | | |
| cg19511338 | 4.21E−09 | 1.79E−03 | −0.213374346 | 0.213374346 | loss | 0.586694581 | 0.373320235 | | 37 | 10 |
| cg27558485 | 7.13E−10 | 3.03E−04 | −0.213363938 | 0.213363938 | loss | 0.717625143 | 0.504261206 | SLC51A | 37 | 3 |
| cg01807131 | 2.12E−08 | 9.01E−03 | −0.213349728 | 0.213349728 | loss | 0.686667275 | 0.473317547 | | 37 | 7 |
| cg06941635 | 9.23E−10 | 3.92E−04 | −0.213307796 | 0.213307796 | loss | 0.798682402 | 0.585374606 | SEPT8; SEPT8; SEPT8; SEPT8 | 37 | 5 |
| cg27319263 | 7.13E−10 | 3.03E−04 | −0.21330015 | 0.21330015 | loss | 0.883435179 | 0.670135029 | NRXN2; NRXN2 | 37 | 11 |
| cg17125472 | 1.10E−09 | 4.65E−04 | −0.213297384 | 0.213297384 | loss | 0.573686049 | 0.360388665 | | 37 | 11 |
| cg02046423 | 7.13E−10 | 3.03E−04 | −0.213198472 | 0.213198472 | loss | 0.435753355 | 0.222554882 | | 37 | 17 |
| cg25946374 | 1.01E−09 | 4.27E−04 | −0.213194423 | 0.213194423 | loss | 0.868882875 | 0.655688453 | IL22RA1; IL22RA1 | 37 | 1 |
| cg23691894 | 1.32E−08 | 5.59E−03 | −0.213165224 | 0.213165224 | loss | 0.871568094 | 0.658402871 | ADD3; ADD3-AS1 | 37 | 10 |
| cg00021600 | 1.07E−07 | 4.55E−02 | −0.213120394 | 0.213120394 | loss | 0.481417247 | 0.268296853 | NECAB2 | 37 | 16 |
| cg22957121 | 7.13E−10 | 3.03E−04 | −0.213111585 | 0.213111585 | loss | 0.643668826 | 0.430557241 | MGAT4C | 37 | 12 |
| cg14023737 | 7.13E−10 | 3.03E−04 | −0.213101238 | 0.213101238 | loss | 0.352940538 | 0.1398393 | NTMT1; NTMT1 | 37 | 9 |
| cg00242839 | 7.77E−10 | 3.30E−04 | −0.213090623 | 0.213090623 | loss | 0.650665906 | 0.437575282 | RBM17; RBM17 | 37 | 10 |
| cg19249231 | 1.67E−08 | 7.10E−03 | −0.213081654 | 0.213081654 | loss | 0.606118766 | 0.393037112 | GPR137B | 37 | 1 |
| cg11976052 | 2.78E−09 | 1.18E−03 | −0.213041881 | 0.213041881 | loss | 0.723941745 | 0.510899865 | KLK4 | 37 | 19 |
| cg15173741 | 7.13E−10 | 3.03E−04 | −0.213035684 | 0.213035684 | loss | 0.868645102 | 0.655609418 | ANK1; ANK1; ANK1; ANK1; | 37 | 8 |
| | | | | | | | | ANK1 | | |
| cg10091408 | 1.30E−09 | 5.52E−04 | −0.213030617 | 0.213030617 | loss | 0.557593106 | 0.344562488 | | 37 | 17 |
| cg04087740 | 3.14E−08 | 1.33E−02 | −0.213017268 | 0.213017268 | loss | 0.341969911 | 0.128952643 | EIF4E3; EIF4E3; GPR27; EIF4E3 | 37 | 3 |
| cg02055264 | 1.43E−08 | 6.06E−03 | −0.213011594 | 0.213011594 | loss | 0.769232294 | 0.5562207 | | 37 | 6 |
| cg05318142 | 7.13E−10 | 3.03E−04 | −0.213005483 | 0.213005483 | loss | 0.29181543 | 0.078809948 | ATOH8 | 37 | 2 |
| cg11323506 | 8.56E−10 | 3.63E−04 | −0.212980515 | 0.212980515 | loss | 0.397529833 | 0.184549318 | CKMT2; CKMT2; CKMT2; RNU5E- | 37 | 5 |
| | | | | | | | | 1; RNU5D-1 | | |
| cg03078169 | 7.13E−10 | 3.03E−04 | −0.212957044 | 0.212957044 | loss | 0.688010185 | 0.475053141 | FXYD1; FXYD1; FXYD1; FXYD1 | 37 | 19 |
| cg26787020 | 8.47E−10 | 3.60E−04 | −0.212955324 | 0.212955324 | loss | 0.532533736 | 0.319578412 | CACYBP; CACYBP | 37 | 1 |
| cg23136738 | 7.13E−10 | 3.03E−04 | −0.212945049 | 0.212945049 | loss | 0.427786349 | 0.2148413 | AP2A2; AP2A2 | 37 | 11 |
| cg22017733 | 1.83E−09 | 7.75E−04 | 0.212943735 | 0.212943735 | gain | 0.3030248 | 0.515968535 | MAT2B; MAT2B | 37 | 5 |
| cg26090062 | 1.68E−09 | 7.12E−04 | −0.212897351 | 0.212897351 | loss | 0.522283998 | 0.309386647 | | 37 | 6 |
| cg00605362 | 3.02E−09 | 1.28E−03 | −0.212891053 | 0.212891053 | loss | 0.433404606 | 0.220513553 | C16orf71 | 37 | 16 |
| cg03104309 | 1.99E−09 | 8.43E−04 | −0.212868573 | 0.212868573 | loss | 0.876742043 | 0.663873471 | LRRC4B | 37 | 19 |
| cg20992663 | 1.30E−09 | 5.52E−04 | −0.212858134 | 0.212858134 | loss | 0.534955487 | 0.322097353 | TRIM33; TRIM33 | 37 | 1 |
| cg20903160 | 1.10E−09 | 4.65E−04 | −0.212853474 | 0.212853474 | loss | 0.350704596 | 0.137851122 | ECT2L; ECT2L | 37 | 6 |
| cg26794871 | 7.13E−10 | 3.03E−04 | −0.212845336 | 0.212845336 | loss | 0.831210794 | 0.618365459 | CDH22 | 37 | 20 |
| cg16899498 | 7.13E−10 | 3.03E−04 | −0.21283198 | 0.21283198 | loss | 0.337009751 | 0.124177771 | MAFA | 37 | 8 |
| cg12350592 | 8.11E−09 | 3.44E−03 | −0.212816705 | 0.212816705 | loss | 0.69580107 | 0.382984365 | PYY | 37 | 17 |
| cg24599996 | 7.13E−10 | 3.03E−04 | −0.212815885 | 0.212815885 | loss | 0.824710932 | 0.611895047 | CELSR2 | 37 | 1 |
| cg04757499 | 1.99E−09 | 8.43E−04 | −0.21279137 | 0.21279137 | loss | 0.789153511 | 0.576362141 | RGR; RGR; RGR | 37 | 10 |
| cg25972714 | 1.73E−09 | 7.34E−04 | −0.212762309 | 0.212762309 | loss | 0.850703509 | 0.6379412 | GRID1; MIR346; MIR346 | 37 | 10 |
| cg10806711 | 7.13E−10 | 3.03E−04 | −0.212746652 | 0.212746652 | loss | 0.334109849 | 0.121363197 | FAM19A3; FAM19A3 | 37 | 1 |
| cg03655395 | 6.35E−09 | 2.70E−03 | −0.212733001 | 0.212733001 | loss | 0.52178073 | 0.309047729 | | 37 | 1 |
| cg20654619 | 7.13E−10 | 3.03E−04 | −0.212723928 | 0.212723928 | loss | 0.858225892 | 0.645501965 | | 37 | 10 |
| cg25744277 | 7.13E−10 | 3.03E−04 | −0.212720613 | 0.212720613 | loss | 0.749665208 | 0.536944594 | OCA2 | 37 | 15 |
| cg26921881 | 1.10E−09 | 4.65E−04 | −0.212702619 | 0.212702619 | loss | 0.92972636 | 0.717023741 | | 37 | 17 |
| cg12226028 | 1.54E−09 | 6.54E−04 | −0.212701302 | 0.212701302 | loss | 0.316572415 | 0.103871114 | C15orf27 | 37 | 15 |
| cg05129477 | 7.13E−10 | 3.03E−04 | −0.212671917 | 0.212671917 | loss | 0.555813511 | 0.343141594 | KIF17; KIF17 | 37 | 1 |
| cg22392793 | 7.13E−10 | 3.03E−04 | −0.212653819 | 0.212653819 | loss | 0.80294766 | 0.590293841 | | 37 | 2 |
| cg18043888 | 7.13E−10 | 3.03E−04 | −0.212621785 | 0.212621785 | loss | 0.364993545 | 0.15237176 | RHOD | 37 | 11 |
| cg19179099 | 1.83E−09 | 7.75E−04 | −0.212613635 | 0.212613635 | loss | 0.617839806 | 0.405226171 | | 37 | 10 |
| cg08319294 | 3.02E−09 | 1.28E−03 | −0.212592682 | 0.212592682 | loss | 0.477197406 | 0.264604724 | | 37 | 19 |
| cg26046772 | 1.68E−09 | 7.12E−04 | −0.212591999 | 0.212591999 | loss | 0.678648475 | 0.466056476 | | 37 | 7 |
| cg20752312 | 5.61E−09 | 2.38E−03 | −0.212574032 | 0.212574032 | loss | 0.652245673 | 0.439671641 | ALKBH7 | 37 | 19 |
| cg22700880 | 9.23E−10 | 3.92E−04 | −0.212573631 | 0.212573631 | loss | 0.595635308 | 0.383061676 | | 37 | 13 |
| cg25975485 | 7.13E−10 | 3.03E−04 | −0.212563651 | 0.212563651 | loss | 0.420402004 | 0.207838353 | HOXC4; HOXC4 | 37 | 12 |
| cg17621438 | 2.35E−09 | 9.98E−04 | −0.212559502 | 0.212559502 | loss | 0.459005091 | 0.246445588 | RNF180; RNF180 | 37 | 5 |
| cg16484042 | 6.89E−09 | 2.93E−03 | −0.21252974 | 0.21252974 | loss | 0.449627381 | 0.237097641 | | 37 | 16 |
| cg24018756 | 3.14E−08 | 1.33E−02 | −0.212528682 | 0.212528682 | loss | 0.45844587 | 0.245917188 | IFITM5 | 37 | 11 |
| cg10683615 | 7.13E−10 | 3.03E−04 | −0.212526482 | 0.212526482 | loss | 0.649385653 | 0.436859171 | INMT; INMT; INMT- | 37 | 7 |
| | | | | | | | | FAM188B | | |
| cg00754947 | 7.13E−10 | 3.03E−04 | −0.212421731 | 0.212421731 | loss | 0.750294513 | 0.537872782 | GRB10 | 37 | 7 |
| cg20392764 | 1.28E−08 | 5.44E−03 | −0.212407521 | 0.212407521 | loss | 0.811979704 | 0.599572182 | ASCL2 | 37 | 11 |
| cg23846712 | 8.11E−09 | 3.44E−03 | −0.212406843 | 0.212406843 | loss | 0.504229766 | 0.291822924 | TBXA2R; TBXA2R; CACTIN- | 37 | 19 |
| | | | | | | | | AS1 | | |
| cg13790268 | 7.77E−10 | 3.30E−04 | −0.212398552 | 0.212398552 | loss | 0.704403481 | 0.492004929 | HHIPL1; HHIPL1 | 37 | 14 |
| cg21100328 | 5.39E−09 | 2.29E−03 | 0.212347089 | 0.212347089 | gain | 0.213730423 | 0.426077512 | DKFZp434J0226 | 37 | 19 |
| cg12404281 | 2.45E−09 | 1.04E−03 | −0.212336744 | 0.212336744 | loss | 0.636185438 | 0.423848694 | RXRG; RXRG; RXRG | 37 | 1 |
| cg12279688 | 1.54E−08 | 6.56E−03 | −0.212330504 | 0.212330504 | loss | 0.775994409 | 0.563663906 | | 37 | 4 |
| cg27274604 | 1.68E−09 | 7.12E−04 | −0.212311867 | 0.212311867 | loss | 0.720448885 | 0.508137018 | SLIT1; ARHGAP19-SLIT1 | 37 | 10 |
| cg09938664 | 7.13E−10 | 3.03E−04 | −0.212237358 | 0.212237358 | loss | 0.494818323 | 0.282580965 | | 37 | 11 |
| cg22566355 | 7.13E−10 | 3.03E−04 | −0.212179054 | 0.212179054 | loss | 0.44036243 | 0.228183376 | KCNH3 | 37 | 12 |
| cg18950952 | 7.13E−10 | 3.03E−04 | −0.212163949 | 0.212163949 | loss | 0.837156191 | 0.624992241 | MALL | 37 | 2 |
| cg07472953 | 2.78E−09 | 1.18E−03 | −0.212157464 | 0.212157464 | loss | 0.751016864 | 0.5388594 | CSF3; CSF3; CSF3; CSF3; CSF3 | 37 | 17 |
| cg09142166 | 5.41E−08 | 2.30E−02 | −0.212142159 | 0.212142159 | loss | 0.661473594 | 0.449331435 | PNPLA5; PNPLA5 | 37 | 22 |
| cg12091331 | 1.12E−08 | 4.76E−03 | −0.212129583 | 0.212129583 | loss | 0.622586325 | 0.410456741 | PLAT; PLAT; PLAT; PLAT | 37 | 8 |
| cg02874016 | 7.13E−10 | 3.03E−04 | −0.212119035 | 0.212119035 | loss | 0.729055853 | 0.516936818 | CACNA2D4 | 37 | 12 |
| cg23526676 | 1.07E−07 | 4.55E−02 | −0.212103111 | 0.212103111 | loss | 0.556393158 | 0.344290047 | VWA7 | 37 | 6 |
| cg26181818 | 4.21E−09 | 1.79E−03 | −0.212102072 | 0.212102072 | loss | 0.616849313 | 0.404747241 | ALPI | 37 | 2 |
| cg21376136 | 8.11E−09 | 3.44E−03 | −0.212095702 | 0.212095702 | loss | 0.410515796 | 0.198420094 | CACNB4; CACNB4; CACNB4; | 37 | 2 |
| | | | | | | | | CACNB4; CACNB4 | | |
| cg03379797 | 3.67E−08 | 1.56E−02 | −0.212086911 | 0.212086911 | loss | 0.714404481 | 0.502317571 | NHSL1 | 37 | 6 |
| cg12758090 | 5.01E−08 | 2.13E−02 | −0.212064019 | 0.212064019 | loss | 0.559066508 | 0.347002488 | | 37 | 15 |
| cg15469871 | 2.30E−08 | 9.75E−03 | −0.212049766 | 0.212049766 | loss | 0.314347036 | 0.102297269 | LRRC37A6P; LRRC37A6P | 37 | 10 |
| cg19343034 | 7.13E−10 | 3.03E−04 | −0.212048498 | 0.212048498 | loss | 0.923173192 | 0.711124694 | MAMSTR | 37 | 19 |
| cg13017345 | 7.77E−10 | 3.30E−04 | −0.212044269 | 0.212044269 | loss | 0.765362728 | 0.553318459 | MXI1 | 37 | 10 |
| cg01328833 | 7.77E−10 | 3.30E−04 | −0.212020478 | 0.212020478 | loss | 0.74899326 | 0.536972782 | SHD | 37 | 19 |
| cg27395056 | 9.23E−10 | 3.92E−04 | −0.212013783 | 0.212013783 | loss | 0.266076302 | 0.054062519 | ACBD4; ACBD4; ACBD4; ACBD4 | 37 | 17 |
| cg01411366 | 1.83E−09 | 7.75E−04 | −0.21200649 | 0.21200649 | loss | 0.472445908 | 0.260439418 | SLC9C1 | 37 | 3 |
| cg21543918 | 7.92E−08 | 3.36E−02 | −0.21200008 | 0.21200008 | loss | 0.764338815 | 0.552338735 | NHSL1 | 37 | 6 |
| cg01908551 | 7.13E−10 | 3.03E−04 | −0.211974937 | 0.211974937 | loss | 0.504778955 | 0.292804018 | METRNL | 37 | 17 |
| cg05123976 | 7.13E−10 | 3.03E−04 | −0.211963929 | 0.211963929 | loss | 0.434284506 | 0.222320576 | | 37 | 17 |
| cg12299599 | 2.56E−09 | 1.09E−03 | −0.211950138 | 0.211950138 | loss | 0.655206874 | 0.443256735 | | 37 | 7 |
| cg10883038 | 7.13E−10 | 3.03E−04 | −0.211925944 | 0.211925944 | loss | 0.373440232 | 0.161514288 | DENND2C; DENND2C | 37 | 1 |
| cg02978784 | 7.13E−10 | 3.03E−04 | −0.211919765 | 0.211919765 | loss | 0.764670789 | 0.552751024 | | 37 | 6 |
| cg17735531 | 1.99E−09 | 8.43E−04 | −0.2119033 | 0.2119033 | loss | 0.625454653 | 0.413551353 | HCN2 | 37 | 19 |
| cg13557773 | 5.16E−09 | 2.19E−03 | −0.211881173 | 0.211881173 | loss | 0.653193585 | 0.441312412 | RASA3 | 37 | 13 |
| cg00531453 | 9.23E−10 | 3.92E−04 | −0.211859369 | 0.211859369 | loss | 0.625711092 | 0.413851724 | TMEM229A | 37 | 7 |
| cg11476737 | 1.68E−09 | 7.12E−04 | −0.211856223 | 0.211856223 | loss | 0.94437197 | 0.732515747 | | 37 | 10 |
| cg23123165 | 1.83E−09 | 7.75E−04 | −0.211845153 | 0.211845153 | loss | 0.829133558 | 0.617288406 | | 37 | 7 |
| cg03708694 | 7.13E−10 | 3.03E−04 | −0.211839011 | 0.211839011 | loss | 0.513304723 | 0.301465712 | | 37 | 11 |
| cg11028624 | 2.35E−09 | 9.98E−04 | −0.211837116 | 0.211837116 | loss | 0.785973787 | 0.574136671 | SPON1 | 37 | 11 |
| cg00909806 | 7.13E−10 | 3.03E−04 | −0.211829742 | 0.211829742 | loss | 0.319491683 | 0.107661941 | | 37 | 1 |
| cg24983120 | 1.44E−09 | 6.12E−04 | −0.211812241 | 0.211812241 | loss | 0.631386906 | 0.419574665 | | 37 | 18 |
| cg27305303 | 2.78E−09 | 1.18E−03 | −0.211783259 | 0.211783259 | loss | 0.4638457 | 0.252062441 | OTOF | 37 | 2 |
| cg17802633 | 2.87E−08 | 1.22E−02 | −0.211783163 | 0.211783163 | loss | 0.544406169 | 0.332623006 | PROKR1 | 37 | 2 |
| cg19551055 | 7.77E−10 | 3.30E−04 | −0.211764224 | 0.211764224 | loss | 0.731165336 | 0.519401112 | | 37 | 11 |
| cg17341345 | 1.10E−09 | 4.65E−04 | −0.211755681 | 0.211755681 | loss | 0.779268345 | 0.567512665 | CCNH; CCNH | 37 | 5 |
| cg05210804 | 2.78E−09 | 1.18E−03 | −0.211754024 | 0.211754024 | loss | 0.360476623 | 0.148722598 | ZBTB22; TAPBP; ZBTB22; TAPBP; | 37 | 6 |
| | | | | | | | | TAPBP | | |
| cg09250458 | 1.01E−09 | 4.27E−04 | −0.211738844 | 0.211738844 | loss | 0.567329691 | 0.355590847 | C1QTNF8 | 37 | 16 |
| cg02787980 | 9.22E−08 | 3.91E−02 | −0.211730148 | 0.211730148 | loss | 0.504703183 | 0.292973035 | PNOC | 37 | 8 |
| cg19793809 | 7.13E−10 | 3.03E−04 | −0.21172976 | 0.21172976 | loss | 0.521735366 | 0.310005606 | SHROOM1; SHROOM1 | 37 | 5 |
| cg06017490 | 1.54E−09 | 6.54E−04 | −0.211728905 | 0.211728905 | loss | 0.607600411 | 0.395871506 | | 37 | 7 |
| cg02792821 | 7.13E−10 | 3.03E−04 | −0.211694499 | 0.211694499 | loss | 0.333953664 | 0.122259165 | | 37 | 20 |
| cg04247717 | 8.47E−10 | 3.60E−04 | −0.21168071 | 0.21168071 | loss | 0.29096863 | 0.079287921 | ARHGEF4; ARHGEF4 | 37 | 2 |
| cg26250093 | 7.13E−10 | 3.03E−04 | −0.211678408 | 0.211678408 | loss | 0.931796296 | 0.720117888 | FAM228A | 37 | 2 |
| cg13692134 | 4.97E−09 | 2.11E−03 | −0.211660449 | 0.211660449 | loss | 0.479712708 | 0.268052259 | | 37 | 5 |
| cg27310219 | 1.12E−08 | 4.76E−03 | −0.211636468 | 0.211636468 | loss | 0.809209921 | 0.597573453 | | 37 | 7 |
| cg25293806 | 1.42E−09 | 6.01E−04 | −0.211625254 | 0.211625254 | loss | 0.517275025 | 0.305649771 | TMEM190 | 37 | 19 |
| cg00031973 | 1.42E−09 | 6.01E−04 | −0.211623112 | 0.211623112 | loss | 0.4075998 | 0.195976688 | | 37 | 7 |
| cg22486928 | 7.13E−10 | 3.03E−04 | −0.211621983 | 0.211621983 | loss | 0.367860372 | 0.156238388 | SNRPF | 37 | 12 |
| cg05730092 | 7.13E−10 | 3.03E−04 | −0.211616863 | 0.211616863 | loss | 0.732320104 | 0.520703241 | BEGAIN | 37 | 14 |
| cg26051413 | 6.30E−08 | 2.68E−02 | −0.211605984 | 0.211605984 | loss | 0.412344749 | 0.200738765 | ASCL2 | 37 | 11 |
| cg04595372 | 1.30E−09 | 5.52E−04 | −0.211604129 | 0.211604129 | loss | 0.690637858 | 0.479033729 | CRISP2; CRISP2; CRISP2; CRISP2; | 37 | 6 |
| | | | | | | | | CRISP2; CRISP2; CRISP2; | | |
| | | | | | | | | CRISP2; CRISP2; CRISP2; | | |
| | | | | | | | | CRISP2; CRISP2 | | |
| cg24378227 | 7.13E−10 | 3.03E−04 | −0.211603932 | 0.211603932 | loss | 0.511492232 | 0.2998883 | | 37 | 6 |
| cg27052709 | 1.03E−08 | 4.39E−03 | −0.211603284 | 0.211603284 | loss | 0.856483996 | 0.644880712 | DNAH17; DNAH17-AS1 | 37 | 17 |
| cg06768010 | 7.13E−10 | 3.03E−04 | −0.211600599 | 0.211600599 | loss | 0.404045181 | 0.192444582 | LBX2; LBX2-AS1 | 37 | 2 |
| cg04788926 | 5.85E−09 | 2.49E−03 | −0.211599919 | 0.211599919 | loss | 0.779450455 | 0.567850535 | | 37 | 18 |
| cg15256387 | 7.13E−10 | 3.03E−04 | −0.211597402 | 0.211597402 | loss | 0.898589943 | 0.686992541 | ETV1; ETV1 | 37 | 7 |
| cg13853761 | 7.13E−10 | 3.03E−04 | −0.211568548 | 0.211568548 | loss | 0.857311236 | 0.645742688 | VPS51; VPS51 | 37 | 11 |
| cg15280185 | 4.57E−09 | 1.94E−03 | −0.21156186 | 0.21156186 | loss | 0.747941372 | 0.536379512 | | 37 | 10 |
| cg14259096 | 9.23E−10 | 3.92E−04 | −0.211560689 | 0.211560689 | loss | 0.708744019 | 0.497183329 | | 37 | 4 |
| cg19218607 | 7.13E−10 | 3.03E−04 | −0.211537686 | 0.211537686 | loss | 0.536060615 | 0.324522929 | | 37 | 12 |
| cg22361859 | 1.30E−09 | 5.52E−04 | −0.211530589 | 0.211530589 | loss | 0.269868162 | 0.058337574 | FBXO47; FBXO47 | 37 | 17 |
| cg08002151 | 1.73E−09 | 7.34E−04 | −0.211518991 | 0.211518991 | loss | 0.460771109 | 0.249252119 | | 37 | 17 |
| cg07181395 | 7.13E−10 | 3.03E−04 | −0.211507921 | 0.211507921 | loss | 0.334415645 | 0.122907724 | | 37 | 2 |
| cg02019125 | 7.13E−10 | 3.03E−04 | −0.211506246 | 0.211506246 | loss | 0.404357817 | 0.192851571 | | 37 | 3 |
| cg19513207 | 1.19E−09 | 5.07E−04 | −0.211496697 | 0.211496697 | loss | 0.437761821 | 0.226265124 | | 37 | 3 |
| cg03379552 | 6.89E−09 | 2.93E−03 | −0.211460612 | 0.211460612 | loss | 0.432679783 | 0.221219171 | CRTAC1; CRTAC1 | 37 | 10 |
| cg02853553 | 7.13E−10 | 3.03E−04 | −0.21139294 | 0.21139294 | loss | 0.778873192 | 0.567480253 | CAMK2B; CAMK2B; CAMK2B; | 37 | 7 |
| | | | | | | | | CAMK2B; CAMK2B; CAMK2B; | | |
| | | | | | | | | CAMK2B; CAMK2B | | |
| cg26459819 | 7.13E−10 | 3.03E−04 | −0.211390436 | 0.211390436 | loss | 0.42436213 | 0.212971694 | COLCA2; COLCA2; COLCA2; | 37 | 11 |
| | | | | | | | | COLCA1 | | |
| cg14555100 | 9.23E−10 | 3.92E−04 | −0.21137942 | 0.21137942 | loss | 0.926301049 | 0.714921629 | GPRIN2 | 37 | 10 |
| cg13422804 | 7.13E−10 | 3.03E−04 | −0.211373292 | 0.211373292 | loss | 0.531022715 | 0.319649424 | AGPAT2; AGPAT2 | 37 | 9 |
| cg12210255 | 7.13E−10 | 3.03E−04 | −0.211345353 | 0.211345353 | loss | 0.296853638 | 0.085508285 | | 37 | 7 |
| cg08144358 | 2.78E−09 | 1.18E−03 | −0.211339797 | 0.211339797 | loss | 0.792239379 | 0.580899582 | | 37 | 7 |
| cg03225580 | 9.23E−10 | 3.92E−04 | −0.211335911 | 0.211335911 | loss | 0.68067837 | 0.469342459 | | 37 | 5 |
| cg26132737 | 8.47E−10 | 3.60E−04 | −0.21133276 | 0.21133276 | loss | 0.731707577 | 0.520374818 | ANKRD44; ANKRD44 | 37 | 2 |
| cg12019614 | 1.16E−07 | 4.91E−02 | 0.211318894 | 0.211318894 | gain | 0.212161759 | 0.423480653 | DOCK6 | 37 | 19 |
| cg11500727 | 1.30E−09 | 5.52E−04 | −0.211306971 | 0.211306971 | loss | 0.448649577 | 0.237342606 | CNFN; CNFN | 37 | 19 |
| cg12638745 | 2.56E−09 | 1.09E−03 | −0.211305677 | 0.211305677 | loss | 0.518789825 | 0.307484147 | SH3BGR; LCA5L | 37 | 21 |
| cg07963854 | 1.68E−09 | 7.12E−04 | −0.211292477 | 0.211292477 | loss | 0.753695942 | 0.542403465 | | 37 | 12 |
| cg12582654 | 7.13E−10 | 3.03E−04 | −0.211278155 | 0.211278155 | loss | 0.430954408 | 0.219676253 | KCP; KCP; KCP; KCP | 37 | 7 |
| cg13700073 | 7.13E−10 | 3.03E−04 | −0.211272493 | 0.211272493 | loss | 0.45091207 | 0.239639576 | FSCN2; FSCN2 | 37 | 17 |
| cg21516302 | 1.30E−09 | 5.52E−04 | −0.211266996 | 0.211266996 | loss | 0.897752396 | 0.6864854 | | 37 | 2 |
| cg06377543 | 1.42E−09 | 6.01E−04 | −0.211238427 | 0.211238427 | loss | 0.761097662 | 0.549859235 | PSMA8; PSMA8; PSMA8 | 37 | 18 |
| cg10194870 | 7.13E−10 | 3.03E−04 | −0.211234478 | 0.211234478 | loss | 0.953069172 | 0.741834694 | OSCAR; OSCAR; OSCAR; OSCAR; | 37 | 19 |
| | | | | | | | | OSCAR; OSCAR | | |
| cg24779381 | 7.77E−10 | 3.30E−04 | −0.211202895 | 0.211202895 | loss | 0.829483289 | 0.618280394 | ISLR; ISLR | 37 | 15 |
| cg04537050 | 7.13E−10 | 3.03E−04 | 0.211172415 | 0.211172415 | loss | 0.825343821 | 0.614171406 | ZSCAN10; ZSCAN10; ZSCAN10; | 37 | 16 |
| | | | | | | | | ZSCAN10 | | |
| cg22533683 | 7.13E−10 | 3.03E−04 | −0.211168805 | 0.211168805 | loss | 0.571392334 | 0.360223529 | MIR193A | 37 | 17 |
| cg20003638 | 1.01E−09 | 4.27E−04 | −0.211094723 | 0.211094723 | loss | 0.758132617 | 0.547037894 | SLC12A5; SLC12A5 | 37 | 20 |
| cg08739828 | 7.77E−10 | 3.30E−04 | −0.211079722 | 0.211079722 | loss | 0.666789492 | 0.455709771 | MOCOS | 37 | 18 |
| cg02372856 | 1.83E−09 | 7.75E−04 | −0.211074695 | 0.211074695 | loss | 0.89975143 | 0.688676735 | | 37 | 8 |
| cg17437419 | 7.13E−10 | 3.03E−04 | −0.211067215 | 0.211067215 | loss | 0.781260262 | 0.570193047 | LOC440311 | 37 | 15 |
| cg13296371 | 7.13E−10 | 3.03E−04 | −0.211056685 | 0.211056685 | loss | 0.377414879 | 0.166358194 | TMEM128 | 37 | 4 |
| cg10601230 | 4.29E−08 | 1.82E−02 | −0.21098196 | 0.21098196 | loss | 0.899725525 | 0.688743565 | PACRG; PACRG; PACRG | 37 | 6 |
| cg03560996 | 3.67E−08 | 1.56E−02 | −0.210980525 | 0.210980525 | loss | 0.866334502 | 0.655353976 | VWA7 | 37 | 6 |
| cg17564844 | 7.13E−10 | 3.03E−04 | −0.2109697 | 0.2109697 | loss | 0.679685942 | 0.468716241 | GNAZ; RTDR1 | 37 | 22 |
| cg14130039 | 7.13E−10 | 3.03E−04 | −0.210966588 | 0.210966688 | loss | 0.328813853 | 0.117847165 | PPT2; PPT2; PPT2; LOC100507547; | 37 | 6 |
| | | | | | | | | LOC100507547; LOC100507547 | | |
| | | | | | | | | LOC100507547; | | |
| | | | | | | | | PPT2-EGFL8 | | |
| cg00900933 | 7.13E−10 | 3.03E−04 | −0.210954547 | 0.210954547 | loss | 0.458033 | 0.247078453 | ARL5C | 37 | 17 |
| cg02962597 | 2.16E−09 | 9.17E−04 | −0.210919759 | 0.210919759 | loss | 0.520721289 | 0.309801529 | NOS1; NOS1; NOS1; NOS1 | 37 | 12 |
| cg11898431 | 2.56E−09 | 1.09E−03 | −0.210888181 | 0.210888181 | loss | 0.657588987 | 0.446700806 | | 37 | 6 |
| cg18162670 | 7.13E−10 | 3.03E−04 | −0.210887389 | 0.210887389 | loss | 0.459463842 | 0.248576453 | | 37 | 14 |
| cg10001845 | 7.13E−10 | 3.03E−04 | −0.210859856 | 0.210859856 | loss | 0.883737862 | 0.672878006 | | 37 | 13 |
| cg06453241 | 7.13E−10 | 3.03E−04 | −0.210827324 | 0.210827324 | loss | 0.783388147 | 0.572560824 | | 37 | 2 |
| cg22261895 | 7.13E−10 | 3.03E−04 | −0.210768911 | 0.210768911 | loss | 0.731396611 | 0.5206277 | PLBD1 | 37 | 12 |
| cg14691388 | 7.13E−10 | 3.03E−04 | −0.210758283 | 0.210758283 | loss | 0.863129094 | 0.652370812 | KCNK9; KCNK9 | 37 | 8 |
| cg25129541 | 7.13E−10 | 3.03E−04 | −0.210738911 | 0.210738911 | loss | 0.490846411 | 0.2801075 | | 37 | 12 |
| cg23594693 | 7.13E−10 | 3.03E−04 | −0.210735685 | 0.210735685 | loss | 0.660098674 | 0.449362988 | TFEB; TFEB; TFEB; TFEB; TFEB | 37 | 6 |
| cg15192101 | 1.30E−09 | 5.52E−04 | −0.210723184 | 0.210723184 | loss | 0.674290555 | 0.463567371 | | 37 | 4 |
| cg21139046 | 7.77E−10 | 3.30E−04 | −0.210704308 | 0.210704308 | loss | 0.765602149 | 0.554897841 | | 37 | 3 |
| cg22610676 | 1.30E−09 | 5.52E−04 | −0.210684575 | 0.210684575 | loss | 0.426466681 | 0.215782106 | C1orf61 | 37 | 1 |
| cg04289314 | 3.88E−09 | 1.65E−03 | −0.210680764 | 0.210680764 | loss | 0.805708823 | 0.595028059 | | 37 | 2 |
| cg25447806 | 7.77E−10 | 3.30E−04 | −0.210669068 | 0.210669068 | loss | 0.560932662 | 0.350263594 | LINC00880 | 37 | 3 |
| cg27110277 | 7.13E−10 | 3.03E−04 | −0.210616514 | 0.210616514 | loss | 0.631855332 | 0.421238818 | FGF20 | 37 | 8 |
| cg08757448 | 8.47E−10 | 3.60E−04 | −0.210616056 | 0.210616056 | loss | 0.843963574 | 0.633347518 | C10orf32; C10orf32; C10orf32- | 37 | 10 |
| | | | | | | | | ASMT | | |
| cg20495333 | 7.13E−10 | 3.03E−04 | −0.210563715 | 0.210563715 | loss | 0.681679968 | 0.471116253 | INHBB | 37 | 2 |
| cg11503875 | 5.85E−09 | 2.49E−03 | −0.210535963 | 0.210535963 | loss | 0.52217054 | 0.311634576 | | 37 | 22 |
| cg18847009 | 3.57E−09 | 1.52E−03 | −0.210524782 | 0.210524782 | loss | 0.389862755 | 0.179337973 | | 37 | 2 |
| cg05477174 | 7.13E−10 | 3.03E−04 | −0.210506353 | 0.210506353 | loss | 0.603467036 | 0.392960682 | IGDCC3 | 37 | 15 |
| cg15482422 | 1.01E−09 | 4.27E−04 | −0.210482251 | 0.210482251 | loss | 0.599151292 | 0.388669041 | RRAD; RRAD | 37 | 16 |
| cg22235293 | 1.68E−09 | 7.12E−04 | −0.210478665 | 0.210478665 | loss | 0.877050989 | 0.666572324 | C6orf25; C6orf25; C6orf25; | 37 | 6 |
| | | | | | | | | C6orf25; C6orf25; C6orf25 | | |
| cg21125503 | 8.47E−10 | 3.60E−04 | −0.210475633 | 0.210475633 | loss | 0.673686051 | 0.463210418 | | 37 | 12 |
| cg11077026 | 7.13E−10 | 3.03E−04 | −0.210473939 | 0.210473939 | loss | 0.428824168 | 0.218350229 | IGSF9; IGSF9 | 37 | 1 |
| cg26382173 | 8.47E−10 | 3.60E−04 | −0.210446428 | 0.210446428 | loss | 0.665326428 | 0.45458 | TMEM121 | 37 | 14 |
| cg14637470 | 7.13E−10 | 3.03E−04 | −0.210433055 | 0.210433055 | loss | 0.792335096 | 0.581902041 | FAM73B | 37 | 9 |
| cg07827796 | 7.13E−10 | 3.03E−04 | −0.210415783 | 0.210415783 | loss | 0.934302342 | 0.723886559 | WDR88 | 37 | 19 |
| cg06621452 | 7.13E−10 | 3.03E−04 | −0.210411172 | 0.210411172 | loss | 0.906763766 | 0.696352594 | MALL | 37 | 2 |
| cg04264925 | 7.13E−10 | 3.03E−04 | −0.210396466 | 0.210396466 | loss | 0.688030025 | 0.477633559 | | 37 | 5 |
| cg11150807 | 1.83E−09 | 7.75E−04 | −0.210392808 | 0.210392808 | loss | 0.865062396 | 0.654669588 | | 37 | 10 |
| cg12938128 | 7.13E−10 | 3.03E−04 | −0.210390141 | 0.210390141 | loss | 0.861975906 | 0.651585765 | NRXN2; NRXN2 | 37 | 11 |
| cg07576591 | 7.48E−09 | 3.18E−03 | −0.210380156 | 0.210380156 | loss | 0.748824062 | 0.538443906 | | 37 | 5 |
| cg08045932 | 3.57E−09 | 1.52E−03 | −0.21036924 | 0.21036924 | loss | 0.543191781 | 0.332822541 | LOC63930 | 37 | 20 |
| cg13388692 | 1.77E−08 | 7.52E−03 | −0.210368611 | 0.210368611 | loss | 0.476382917 | 0.266014306 | | 37 | 1 |
| cg22998028 | 7.13E−10 | 3.03E−04 | −0.210367344 | 0.210367344 | loss | 0.530422932 | 0.320055588 | ASB16 | 37 | 17 |
| cg06161375 | 8.47E−10 | 3.60E−04 | −0.210314751 | 0.210314751 | loss | 0.695447309 | 0.485132559 | TRIM63 | 37 | 1 |
| cg08350509 | 1.10E−09 | 4.65E−04 | −0.210314166 | 0.210314166 | loss | 0.599146325 | 0.388832159 | TLE2; TLE2; TLE2 | 37 | 19 |
| cg16922763 | 2.06E−09 | 8.75E−04 | −0.210293615 | 0.210293615 | loss | 0.297683355 | 0.08738974 | | 37 | 13 |
| cg08756640 | 7.13E−10 | 3.03E−04 | −0.210261373 | 0.210261373 | loss | 0.564777838 | 0.354516465 | | 37 | 10 |
| cg06722121 | 7.13E−10 | 3.03E−04 | −0.210230513 | 0.210230513 | loss | 0.57761006 | 0.367379547 | OTOP3; OTOP3 | 37 | 17 |
| cg01618988 | 1.19E−09 | 5.07E−04 | −0.210225339 | 0.210225339 | loss | 0.731180698 | 0.520955359 | | 37 | 20 |
| cg09084256 | 1.43E−08 | 6.06E−03 | −0.210223962 | 0.210223962 | loss | 0.756631779 | 0.546407818 | C22orf42; C22orf42 | 37 | 22 |
| cg09134760 | 7.13E−10 | 3.03E−04 | −0.210201108 | 0.210201108 | loss | 0.418226085 | 0.208024976 | | 37 | 5 |
| cg24654266 | 1.12E−08 | 4.76E−03 | −0.21018403 | 0.21018403 | loss | 0.364493992 | 0.154309962 | DPP10; DPP10 | 37 | 2 |
| cg11630226 | 9.54E−09 | 4.05E−03 | −0.210126722 | 0.210126722 | loss | 0.359847772 | 0.14972105 | LY6K; LY6K; LY6K | 37 | 8 |
| cg13717553 | 7.77E−10 | 3.30E−04 | −0.210115329 | 0.210115329 | loss | 0.593989853 | 0.383874524 | LRRC4B | 37 | 19 |
| cg16489360 | 7.13E−10 | 3.03E−04 | −0.210097785 | 0.210097785 | loss | 0.648527691 | 0.438429906 | TMEM209 | 37 | 7 |
| cg07148341 | 4.64E−08 | 1.97E−02 | −0.210094854 | 0.210094854 | loss | 0.852459972 | 0.642365118 | | 37 | 6 |
| cg07088882 | 7.77E−10 | 3.30E−04 | −0.210063094 | 0.210063094 | loss | 0.371691942 | 0.161628847 | | 37 | 11 |
| cg20473155 | 7.13E−10 | 3.03E−04 | −0.210055403 | 0.210055403 | loss | 0.358335321 | 0.148279918 | | 37 | 2 |
| cg10060499 | 2.35E−09 | 9.98E−04 | −0.210047058 | 0.210047058 | loss | 0.822946481 | 0.612899424 | | 37 | X |
| cg10257302 | 8.80E−09 | 3.74E−03 | −0.210046207 | 0.210046207 | loss | 0.434271572 | 0.224225365 | TBPL2; TBPL2 | 37 | 14 |
| cg24302529 | 7.77E−10 | 3.30E−04 | −0.21004561 | 0.21004561 | loss | 0.57757784 | 0.367532229 | ALOX5; ALOX5; ALOX5 | 37 | 10 |
| cg22442730 | 7.13E−10 | 3.03E−04 | −0.210026961 | 0.210026961 | loss | 0.389521243 | 0.179494282 | SRMS | 37 | 20 |
| cg05826295 | 4.57E−09 | 1.94E−03 | −0.210025886 | 0.210025886 | loss | 0.360527757 | 0.150501871 | | 37 | 6 |
| cg12265130 | 5.39E−09 | 2.29E−03 | −0.209997713 | 0.209997713 | loss | 0.51826163 | 0.308263918 | PATL1 | 37 | 11 |
| cg20121927 | 1.83E−09 | 7.75E−04 | −0.209994091 | 0.209994091 | loss | 0.792853326 | 0.582859235 | LINC00473; LINC00473 | 37 | 6 |
| cg03630015 | 1.01E−09 | 4.27E−04 | −0.209979939 | 0.209979939 | loss | 0.772887509 | 0.562907571 | WNT10A | 37 | 2 |
| cg01978213 | 8.47E−10 | 3.60E−04 | −0.20997883 | 0.20997883 | loss | 0.602102047 | 0.392123218 | | 37 | 4 |
| cg01832802 | 7.13E−10 | 3.03E−04 | −0.209921047 | 0.209921047 | loss | 0.723070842 | 0.513149794 | LINC00605 | 37 | 14 |
| cg22807681 | 1.68E−09 | 7.12E−04 | −0.209916345 | 0.209916345 | loss | 0.331857698 | 0.121941354 | | 37 | 5 |
| cg27361011 | 7.13E−10 | 3.03E−04 | −0.209874577 | 0.209874577 | loss | 0.381997494 | 0.172122918 | | 37 | 2 |
| cg05831517 | 1.19E−09 | 5.07E−04 | −0.20986796 | 0.20986796 | loss | 0.865632813 | 0.655764853 | | 37 | 16 |
| cg15062725 | 7.13E−10 | 3.03E−04 | −0.209844626 | 0.209844626 | loss | 0.748864085 | 0.539019459 | ZFR2 | 37 | 19 |
| cg17881600 | 7.13E−10 | 3.03E−04 | −0.209834371 | 0.209834371 | loss | 0.872738336 | 0.662903965 | | 37 | 6 |
| cg21647560 | 8.47E−10 | 3.60E−04 | −0.209833155 | 0.209833155 | loss | 0.641164885 | 0.431331729 | | 37 | 1 |
| cg08069902 | 7.38E−08 | 3.13E−02 | −0.209802293 | 0.209802293 | loss | 0.739387506 | 0.529585213 | KCN10; KCNK10; KCNK10 | 37 | 14 |
| cg17278401 | 7.13E−10 | 3.03E−04 | −0.209801846 | 0.209801846 | loss | 0.401114617 | 0.191312771 | MAST4; MAST4 | 37 | 5 |
| cg20960833 | 1.99E−09 | 8.43E−04 | −0.209773082 | 0.209773082 | loss | 0.639058506 | 0.429285424 | | 37 | 7 |
| cg27269130 | 4.57E−09 | 1.94E−03 | −0.209768218 | 0.209768218 | loss | 0.770555042 | 0.560786824 | CDC42EP5 | 37 | 19 |
| cg12854815 | 1.43E−08 | 6.06E−03 | −0.209760425 | 0.209760425 | loss | 0.773460466 | 0.563700041 | | 37 | 6 |
| cg27241889 | 2.56E−09 | 1.09E−03 | −0.209758396 | 0.209758396 | loss | 0.681606913 | 0.471848518 | ERICH1-AS1; ERICH1-AS1 | 37 | 8 |
| cg15671996 | 7.13E−10 | 3.03E−04 | −0.209745634 | 0.209745634 | loss | 0.715495223 | 0.505749588 | TRIM15 | 37 | 6 |
| cg00157359 | 7.13E−10 | 3.03E−04 | −0.209741723 | 0.209741723 | loss | 0.496454517 | 0.286712794 | PELI3; PELI3; PELI3; PELI3 | 37 | 11 |
| cg01443946 | 1.96E−08 | 8.33E−03 | −0.209733472 | 0.209733472 | loss | 0.816530972 | 0.6067975 | ANO2; ANO2 | 37 | 12 |
| cg20703003 | 1.96E−08 | 8.33E−03 | −0.209731586 | 0.209731586 | loss | 0.777113698 | 0.567382112 | | 37 | 10 |
| cg19956113 | 7.84E−10 | 3.33E−04 | −0.209714111 | 0.209714111 | loss | 0.840934623 | 0.631220512 | CHRD | 37 | 3 |
| cg14319451 | 7.13E−10 | 3.03E−04 | −0.209696071 | 0.209696071 | loss | 0.447685 | 0.237988929 | RCOR2 | 37 | 11 |
| cg22318514 | 4.29E−08 | 1.82E−02 | −0.209686671 | 0.209686671 | loss | 0.381067938 | 0.171381266 | | 37 | 6 |
| cg11787508 | 2.48E−08 | 1.05E−02 | −0.209673438 | 0.209673438 | loss | 0.758489932 | 0.548816494 | | 37 | 13 |
| cg23598212 | 1.30E−09 | 5.52E−04 | −0.20963266 | 0.20963266 | loss | 0.708759977 | 0.499127318 | IFITM10; MOB2 | 37 | 11 |
| cg14546466 | 1.30E−09 | 5.52E−04 | −0.20962128 | 0.20962128 | loss | 0.595769698 | 0.386148418 | | 37 | 2 |
| cg22535729 | 4.21E−09 | 1.79E−03 | −0.209600039 | 0.209600039 | loss | 0.927280809 | 0.717680771 | RP1 | 37 | 8 |
| cg01518016 | 7.13E−10 | 3.03E−04 | −0.209599532 | 0.209599532 | loss | 0.795234374 | 0.585634841 | SYCN | 37 | 19 |
| cg22395765 | 3.88E−09 | 1.65E−03 | −0.209547244 | 0.209547244 | loss | 0.618312285 | 0.408765041 | PTPRN2; PTPRN2; PTPRN2 | 37 | 7 |
| cg08577298 | 7.13E−10 | 3.03E−04 | −0.209525438 | 0.209525438 | loss | 0.846568091 | 0.637042653 | | 37 | 5 |
| cg27053299 | 5.39E−09 | 2.29E−03 | −0.209519921 | 0.209519921 | loss | 0.621727726 | 0.412207806 | CLYBL | 37 | 13 |
| cg27570575 | 4.57E−09 | 1.94E−03 | −0.209514138 | 0.209514138 | loss | 0.805885874 | 0.596371735 | | 37 | 16 |
| cg18324432 | 7.13E−10 | 3.03E−04 | −0.209507529 | 0.209507529 | loss | 0.627420306 | 0.417912776 | | 37 | 17 |
| cg17672101 | 7.13E−10 | 3.03E−04 | −0.209491104 | 0.209491104 | loss | 0.877778198 | 0.668287094 | ZFR2 | 37 | 19 |
| cg26256793 | 1.54E−09 | 6.54E−04 | −0.209479366 | 0.209479366 | loss | 0.388812572 | 0.179333206 | COL11A1; COL11A1; COL11A1; | 37 | 1 |
| | | | | | | | | COL11A1 | | |
| cg10210594 | 1.10E−09 | 4.65E−04 | −0.209406812 | 0.209406812 | loss | 0.230968065 | 0.021561253 | | 37 | 1 |
| cg03432955 | 8.47E−10 | 3.60E−04 | −0.209394185 | 0.209394185 | loss | 0.427643338 | 0.218249153 | HLA-G | 37 | 6 |
| cg00593462 | 2.16E−09 | 9.17E−04 | −0.209356698 | 0.209356698 | loss | 0.608140204 | 0.398783506 | | 37 | 13 |
| cg13329862 | 7.13E−10 | 3.03E−04 | −0.209353736 | 0.209353736 | loss | 0.81784943 | 0.608495694 | DDR1; DDR1 | 37 | 6 |
| cg23178550 | 7.13E−10 | 3.03E−04 | −0.209351198 | 0.209351198 | loss | 0.349142274 | 0.139791076 | | 37 | 7 |
| cg25913761 | 1.12E−08 | 4.76E−03 | −0.20934663 | 0.20934663 | loss | 0.664220242 | 0.454873612 | SEMA4B | 37 | 15 |
| cg25480418 | 7.13E−10 | 3.03E−04 | −0.209329428 | 0.209329428 | loss | 0.351845026 | 0.142515599 | | 37 | 2 |
| cg12955789 | 1.83E−09 | 7.75E−04 | −0.209310661 | 0.209310561 | loss | 0.728961185 | 0.519650524 | RASGEF1C | 37 | 5 |
| cg25458010 | 7.77E−10 | 3.30E−04 | −0.209307052 | 0.209307052 | loss | 0.79085307 | 0.581546018 | GALNT9 | 37 | 12 |
| cg10905012 | 7.13E−10 | 3.03E−04 | −0.209293255 | 0.209293255 | loss | 0.872614491 | 0.663321235 | NRXN2; NRXN2; NRXN2 | 37 | 11 |
| cg10414829 | 7.77E−10 | 3.30E−04 | −0.209276287 | 0.209276287 | loss | 0.52977344 | 0.320497153 | SNCG | 37 | 10 |
| cg02013987 | 7.13E−10 | 3.03E−04 | −0.209245547 | 0.209245547 | loss | 0.863355347 | 0.6541098 | ZNF222; ZNF222 | 37 | 19 |
| cg09988676 | 1.68E−09 | 7.12E−04 | −0.20923934 | 0.20923934 | loss | 0.732705992 | 0.523466653 | CHRND; CHRND; CHRND; CHRND | 37 | 2 |
| cg18825119 | 4.29E−08 | 1.82E−02 | −0.20921864 | 0.20921864 | loss | 0.806496134 | 0.597277494 | PACRG; PACRG; PARK2; PARK2; | 37 | 6 |
| | | | | | | | | PARK2; PACRG | | |
| cg18001869 | 7.13E−10 | 3.03E−04 | −0.209158257 | 0.209158257 | loss | 0.760883187 | 0.551724929 | CACNA1I; CACNA1I | 37 | 22 |
| cg25877436 | 1.99E−09 | 8.43E−04 | −0.20915375 | 0.20915375 | loss | 0.585637479 | 0.376483729 | GTF3A | 37 | 13 |
| cg07774964 | 4.57E−09 | 1.94E−03 | −0.209147271 | 0.209147271 | loss | 0.692902842 | 0.483755571 | PLEKHO2; PLEKHO2 | 37 | 15 |
| cg19093295 | 7.13E−10 | 3.03E−04 | −0.209138818 | 0.209138818 | loss | 0.798500394 | 0.589361576 | | 37 | 11 |
| cg18735956 | 7.13E−10 | 3.03E−04 | −0.209136382 | 0.209136382 | loss | 0.266449434 | 0.057313052 | GRM4; GRM4; GRM4; GRM4; | 37 | 6 |
| | | | | | | | | GRM4; GRM4; GRM4 | | |
| cg22380178 | 7.13E−10 | 3.03E−04 | −0.209119898 | 0.209119898 | loss | 0.507995845 | 0.298875947 | | 37 | 15 |
| cg10145449 | 7.13E−10 | 3.03E−04 | −0.209078135 | 0.209078135 | loss | 0.969353747 | 0.760275612 | SPATC1; SPATC1 | 37 | 8 |
| cg04094751 | 7.13E−10 | 3.03E−04 | −0.209056815 | 0.209056815 | gain | 0.109479762 | 0.318536576 | HSPA12B; HSPA12B | 37 | 20 |
| cg14182974 | 9.23E−10 | 3.92E−04 | −0.209055606 | 0.209055606 | loss | 0.300604351 | 0.091548745 | RCL1; RCL1; RCL1 | 37 | 9 |
| cg16436164 | 8.47E−10 | 3.60E−04 | −0.209046656 | 0.209046656 | loss | 0.670479821 | 0.461433165 | | 37 | 12 |
| cg04768368 | 9.23E−10 | 3.92E−04 | −0.209015457 | 0.209015457 | loss | 0.787838251 | 0.578822794 | TACC2; TACC2 | 37 | 10 |
| cg09200774 | 7.13E−10 | 3.03E−04 | −0.20898685 | 0.20898685 | loss | 0.834459032 | 0.625472182 | | 37 | 15 |
| cg14584255 | 2.91E−08 | 1.23E−02 | −0.208986665 | 0.208986665 | loss | 0.73355853 | 0.524571865 | PACRG; PACRG; PARK2; PARK2; | 37 | 6 |
| | | | | | | | | PARK2; PACRG | | |
| cg23258611 | 7.13E−10 | 3.03E−04 | −0.208967077 | 0.208967077 | loss | 0.34936086 | 0.140393784 | LINC00673 | 37 | 17 |
| cg21697944 | 7.13E−10 | 3.03E−04 | −0.208953883 | 0.208953883 | loss | 0.720360036 | 0.511406153 | HRC; HRC | 37 | 19 |
| cg15860114 | 7.13E−10 | 3.03E−04 | −0.208947506 | 0.208947506 | loss | 0.845822606 | 0.6368751 | | 37 | 10 |
| cg11437810 | 7.10E−10 | 3.02E−04 | −0.208943916 | 0.208943916 | loss | 0.217154724 | 0.008210808 | RPLP1; RPLP1 | 37 | 15 |
| cg05313129 | 2.56E−09 | 1.09E−03 | −0.208936368 | 0.208936368 | loss | 0.595884726 | 0.386948359 | LINC00588 | 37 | 8 |
| cg02481697 | 8.47E−10 | 3.60E−04 | −0.208933478 | 0.203933478 | loss | 0.440548949 | 0.231615471 | NTN1 | 37 | 17 |
| cg11886639 | 4.57E−09 | 1.94E−03 | −0.208896639 | 0.208896639 | loss | 0.857214662 | 0.648318024 | | 37 | 8 |
| cg05264816 | 7.13E−10 | 3.03E−04 | −0.20887945 | 0.20887945 | loss | 0.872716568 | 0.663837118 | LOXHD1; LOXHD1 | 37 | 18 |
| cg25593625 | 7.13E−10 | 3.03E−04 | −0.208866099 | 0.208866099 | loss | 0.397839417 | 0.188973318 | MYOM3 | 37 | 1 |
| cg16225804 | 5.39E−09 | 2.29E−03 | −0.208853763 | 0.208853763 | loss | 0.506139504 | 0.297285741 | | 37 | 15 |
| cg15836046 | 7.13E−10 | 3.03E−04 | −0.208836733 | 0.208836733 | loss | 0.856942568 | 0.648105835 | | 37 | 1 |
| cg15174248 | 7.13E−10 | 3.03E−04 | −0.20882148 | 0.20882148 | loss | 0.472820498 | 0.263999018 | CAMK2A; CAMK2A | 37 | 5 |
| cg14437996 | 7.13E−10 | 3.03E−04 | −0.208793141 | 0.208793141 | loss | 0.498111764 | 0.289318624 | SHC3 | 37 | 9 |
| cg09136878 | 5.41E−08 | 2.30E−02 | −0.208792087 | 0.208792087 | loss | 0.414680904 | 0.205888816 | | 37 | 1 |
| cg14227911 | 1.22E−08 | 5.16E−03 | −0.208776986 | 0.208776986 | loss | 0.720074509 | 0.511297524 | | 37 | 13 |
| cg14254748 | 1.96E−08 | 8.33E−03 | −0.208761395 | 0.208761395 | loss | 0.458342189 | 0.249580794 | L1TD1; L1TD1 | 37 | 1 |
| cg12564962 | 7.13E−10 | 3.03E−04 | −0.208735096 | 0.208735096 | loss | 0.905150508 | 0.696415412 | RIPPLY3 | 37 | 21 |
| cg00310410 | 1.99E−09 | 8.43E−04 | −0.208715873 | 0.208715873 | loss | 0.776224432 | 0.567508559 | | 37 | 2 |
| cg12146150 | 7.13E−10 | 3.03E−04 | −0.208686898 | 0.208686898 | loss | 0.901313098 | 0.6926262 | NTN1 | 37 | 17 |
| cg01123965 | 2.56E−09 | 1.09E−03 | −0.208659767 | 0.208659767 | loss | 0.466589151 | 0.257929384 | | 37 | X |
| cg03176729 | 1.44E−08 | 6.09E−03 | −0.208626853 | 0.208626853 | loss | 0.711206247 | 0.502579394 | HTRA1 | 37 | 10 |
| cg01806972 | 7.13E−10 | 3.03E−04 | −0.208605978 | 0.208605978 | loss | 0.298637755 | 0.090031776 | ACTN3; ACTN3; ZDHHC24; | 37 | 11 |
| | | | | | | | | ACTN3 | | |
| cg23132774 | 2.56E−09 | 1.09E−03 | −0.208570963 | 0.208570963 | loss | 0.65702494 | 0.448453976 | RASA3 | 37 | 13 |
| cg19871173 | 1.03E−08 | 4.39E−03 | −0.208538826 | 0.208538826 | loss | 0.843185679 | 0.634646853 | | 37 | 1 |
| cg14508358 | 2.56E−09 | 1.09E−03 | −0.208507308 | 0.208507308 | loss | 0.301422689 | 0.092915381 | | 37 | 9 |
| cg23063807 | 7.13E−10 | 3.03E−04 | −0.208501361 | 0.208501361 | loss | 0.442012543 | 0.233511182 | | 37 | 2 |
| cg06662140 | 2.78E−09 | 1.18E−03 | −0.208479766 | 0.208479766 | loss | 0.950573319 | 0.742093553 | KLHDC7A | 37 | 1 |
| cg04671739 | 7.13E−10 | 3.03E−04 | −0.208444971 | 0.208444971 | loss | 0.634194677 | 0.425749706 | ASB10; ASB10; ASB10; ASB10 | 37 | 7 |
| cg03522256 | 1.01E−09 | 4.27E−04 | −0.208431656 | 0.208431656 | loss | 0.579182479 | 0.370750824 | | 37 | 12 |
| cg06533798 | 7.13E−10 | 3.03E−04 | −0.208403807 | 0.208403807 | loss | 0.61556276 | 0.407158953 | | 37 | 11 |
| cg25498045 | 1.54E−09 | 6.54E−04 | −0.208372264 | 0.208372264 | loss | 0.597116964 | 0.3887447 | GAPDHS | 37 | 19 |
| cg19252333 | 7.13E−10 | 3.03E−04 | −0.208364501 | 0.208364501 | loss | 0.671757077 | 0.463392576 | PTPRU; PTPRU; PTPRU; PTPRU | 37 | 1 |
| cg17359265 | 7.13E−10 | 3.03E−04 | −0.20835094 | 0.20835094 | loss | 0.585936992 | 0.377586053 | ADAMTS2 | 37 | 5 |
| cg24439713 | 7.13E−10 | 3.03E−04 | −0.208349855 | 0.208349855 | loss | 0.657502208 | 0.449152353 | | 37 | 1 |
| cg22177286 | 8.11E−09 | 3.44E−03 | −0.208332833 | 0.208332833 | loss | 0.734334051 | 0.526001218 | ITM2C; ITM2C; ITM2C | 37 | 2 |
| cg08541962 | 1.67E−08 | 7.10E−03 | −0.208325028 | 0.208325028 | loss | 0.646299092 | 0.437974065 | GJD4 | 37 | 10 |
| cg02059315 | 7.13E−10 | 3.03E−04 | −0.208323554 | 0.208323554 | loss | 0.632364713 | 0.424041159 | | 37 | 16 |
| cg20160695 | 7.13E−10 | 3.03E−04 | −0.208305764 | 0.208305764 | loss | 0.768157711 | 0.559851947 | | 37 | 6 |
| cg24324441 | 7.13E−10 | 3.03E−04 | −0.2082964 | 0.2082964 | loss | 0.317429192 | 0.109132792 | MFSD1; MFSD1 | 37 | 3 |
| cg15463284 | 3.02E−09 | 1.28E−03 | −0.208293053 | 0.208293053 | loss | 0.814044553 | 0.6057515 | LDHAL6A; LDHAL6A | 37 | 11 |
| cg12462883 | 7.77E−10 | 3.30E−04 | −0.208292842 | 0.208292842 | loss | 0.881573742 | 0.6732809 | SLC9C1; SLC9C1 | 37 | 3 |
| cg23711873 | 7.13E−10 | 3.03E−04 | −0.203277165 | 0.208277165 | loss | 0.303026521 | 0.094749356 | | 37 | 10 |
| cg13947929 | 7.13E−10 | 3.03E−04 | −0.208266748 | 0.208266748 | loss | 0.459232677 | 0.250965929 | CRHR1; CRHR1; CRHR1; CRHR1; | 37 | 17 |
| | | | | | | | | CRHR1 | | |
| cg03312205 | 1.68E−09 | 7.12E−04 | −0.208256987 | 0.208256987 | loss | 0.73349964 | 0.525242653 | COPS7A; COPS7A; COPS7A; | 37 | 12 |
| | | | | | | | | COPS7A | | |
| cg14554415 | 1.32E−08 | 5.59E−03 | −0.208243311 | 0.208243311 | loss | 0.671427458 | 0.463184147 | NOX5; SPESP1; MIR548H4; | 37 | 15 |
| | | | | | | | | NOX5; NOX5 | | |
| cg06067425 | 7.13E−10 | 3.03E−04 | −0.20824171 | 0.20824171 | loss | 0.510716387 | 0.302474676 | LOC152586 | 37 | 4 |
| cg22325294 | 7.13E−10 | 3.03E−04 | −0.208223643 | 0.208223643 | loss | 0.566252466 | 0.358028824 | RNF39; RNF39 | 37 | 6 |
| cg27511599 | 1.68E−09 | 7.12E−04 | −0.208210714 | 0.208210714 | loss | 0.426091443 | 0.217880729 | | 37 | 7 |
| cg20639805 | 4.57E−09 | 1.94E−03 | −0.208201685 | 0.208201685 | loss | 0.390710938 | 0.182509253 | RBM20 | 37 | 10 |
| cg06312846 | 1.19E−09 | 5.07E−04 | −0.208182758 | 0.208182758 | loss | 0.384363458 | 0.1761807 | | 37 | 6 |
| cg27666046 | 1.30E−09 | 5.52E−04 | −0.208180369 | 0.208180369 | loss | 0.577378945 | 0.369198576 | SECTM1 | 37 | 17 |
| cg02465887 | 1.67E−08 | 7.10E−03 | −0.208177429 | 0.208177429 | loss | 0.8962642 | 0.688086771 | DCHS2 | 37 | 4 |
| cg06061081 | 1.32E−08 | 5.59E−03 | −0.208134077 | 0.208134077 | loss | 0.759445089 | 0.551311012 | AQP11 | 37 | 11 |
| cg27424326 | 7.13E−10 | 3.03E−04 | −0.208121565 | 0.208121565 | loss | 0.885710383 | 0.677588818 | | 37 | 2 |
| cg12067764 | 4.57E−09 | 1.94E−03 | −0.208108327 | 0.208108327 | loss | 0.32478457 | 0.116676242 | | 37 | 1 |
| cg08052629 | 2.16E−09 | 9.17E−04 | −0.208089818 | 0.208089818 | loss | 0.71582503 | 0.507735212 | PPP1R14A; PPP1R14A | 37 | 19 |
| cg26557756 | 7.77E−10 | 3.30E−04 | −0.208050797 | 0.208050797 | loss | 0.547319968 | 0.339269171 | GLI3 | 37 | 7 |
| cg14036033 | 7.77E−10 | 3.30E−04 | −0.208047206 | 0.208047206 | loss | 0.449307253 | 0.241260047 | | 37 | 1 |
| cg07241660 | 1.12E−08 | 4.76E−03 | −0.208045397 | 0.208045397 | loss | 0.719153085 | 0.511107688 | | 37 | 11 |
| cg23878875 | 7.13E−10 | 3.03E−04 | −0.208030678 | 0.208030678 | loss | 0.754933972 | 0.546903294 | XYLT2 | 37 | 17 |
| cg09265392 | 7.13E−10 | 3.03E−04 | −0.208011491 | 0.208011491 | loss | 0.338006645 | 0.129995154 | | 37 | 16 |
| cg02276974 | 6.30E−08 | 2.68E−02 | −0.208005455 | 0.208005455 | loss | 0.559671649 | 0.351666194 | CRABP1 | 37 | 15 |
| cg22068464 | 9.23E−10 | 3.92E−04 | −0.207998205 | 0.207998205 | loss | 0.854439717 | 0.646441512 | | 37 | 2 |
| cg14214165 | 2.78E−09 | 1.18E−03 | −0.207991391 | 0.207991391 | loss | 0.649581538 | 0.441590147 | TNS1 | 37 | 2 |
| cg04142296 | 8.11E−09 | 3.44E−03 | −0.207985026 | 0.207985026 | loss | 0.372922096 | 0.164937071 | PLEKHA2 | 37 | 8 |
| cg11235602 | 7.34E−08 | 3.12E−02 | −0.207984739 | 0.207984739 | loss | 0.354829025 | 0.146844286 | MOBP; MOBP; MOBP; MOBP; | 37 | 3 |
| | | | | | | | | MOBP; MOBP; MOBP | | |
| cg16243197 | 8.47E−10 | 3.60E−04 | −0.207980066 | 0.207980066 | loss | 0.870815213 | 0.662835147 | DHX9; DHX9 | 37 | 1 |
| cg14026325 | 5.01E−08 | 2.13E−02 | −0.207964371 | 0.207964371 | loss | 0.930803747 | 0.722839376 | | 37 | 8 |
| cg23810282 | 1.83E−09 | 7.75E−04 | −0.207953921 | 0.207953921 | loss | 0.783916768 | 0.575962847 | | 37 | 5 |
| cg09857070 | 1.83E−09 | 7.75E−04 | −0.20795157 | 0.20795157 | loss | 0.621676553 | 0.413724982 | | 37 | 1 |
| cg04197086 | 1.68E−09 | 7.12E−04 | −0.207937504 | 0.207937504 | loss | 0.757754145 | 0.549816641 | | 37 | 13 |
| cg17207000 | 7.13E−10 | 3.03E−04 | −0.207928771 | 0.207928771 | loss | 0.475954547 | 0.268025776 | KIF19 | 37 | 17 |
| cg21356710 | 1.30E−09 | 5.52E−04 | −0.207876885 | 0.207876885 | loss | 0.593585774 | 0.385708888 | MFSD2B | 37 | 2 |
| cg00871787 | 7.13E−10 | 3.03E−04 | −0.207854114 | 0.207854114 | loss | 0.924670949 | 0.716816835 | | 37 | 17 |
| cg25882597 | 7.13E−10 | 3.03E−04 | −0.207838178 | 0.207838178 | loss | 0.541363813 | 0.333525635 | LPIN3 | 37 | 20 |
| cg25849642 | 4.57E−09 | 1.94E−03 | −0.207811142 | 0.207811142 | loss | 0.616424983 | 0.408613841 | | 37 | 13 |
| cg22858288 | 1.83E−09 | 7.75E−04 | −0.207799385 | 0.207799385 | loss | 0.724656438 | 0.516857053 | SLC22A18; SLC22A18 | 37 | 11 |
| cg05692746 | 7.77E−10 | 3.30E−04 | −0.207796662 | 0.207796662 | loss | 0.602190426 | 0.394393765 | LONRF2 | 37 | 2 |
| cg19399165 | 8.47E−10 | 3.60E−04 | −0.20775414 | 0.20775414 | loss | 0.768532387 | 0.560778247 | EMILIN1 | 37 | 2 |
| cg09807524 | 7.77E−10 | 3.30E−04 | −0.207747148 | 0.207747148 | loss | 0.807479766 | 0.599732618 | OTOG | 37 | 11 |
| cg20733761 | 2.48E−08 | 1.05E−02 | −0.207734312 | 0.207734312 | loss | 0.755375236 | 0.547640924 | PCBP3; PCBP3 | 37 | 21 |
| cg19683073 | 7.77E−10 | 3.30E−04 | −0.207689745 | 0.207689745 | loss | 0.822645151 | 0.614955406 | SERINC5; SERINC5; SERINC5 | 37 | 5 |
| cg19257464 | 7.13E−10 | 3.03E−04 | −0.207665117 | 0.207665117 | loss | 0.823029106 | 0.615363988 | | 37 | 2 |
| cg06531916 | 8.47E−10 | 3.60E−04 | −0.207648608 | 0.207648608 | loss | 0.881244585 | 0.673595976 | | 37 | 12 |
| cg04010205 | 7.13E−10 | 3.03E−04 | −0.207632984 | 0.207632984 | loss | 0.761795208 | 0.554162224 | GLIS3; GLIS3 | 37 | 9 |
| cg24546723 | 7.13E−10 | 3.03E−04 | −0.207615616 | 0.207615616 | loss | 0.535223492 | 0.327607876 | DLX4 | 37 | 17 |
| cg02050426 | 2.78E−09 | 1.18E−03 | −0.207591898 | 0.207591898 | loss | 0.759829245 | 0.552237347 | CDH20 | 37 | 18 |
| cg07643267 | 1.01E−09 | 4.27E−04 | −0.207571491 | 0.207571491 | loss | 0.472257891 | 0.2646864 | LYPD3; LYPD3 | 37 | 19 |
| cg10287786 | 7.48E−09 | 3.18E−03 | −0.207548079 | 0.207548079 | loss | 0.538770885 | 0.331222806 | DSCAML1 | 37 | 11 |
| cg05609335 | 8.47E−10 | 3.60E−04 | −0.207546492 | 0.207546492 | loss | 0.546315809 | 0.338769318 | | 37 | 17 |
| cg07348481 | 7.13E−10 | 3.03E−04 | −0.207541549 | 0.207541549 | loss | 0.387975613 | 0.180434065 | | 37 | 11 |
| cg02055607 | 7.13E−10 | 3.03E−04 | −0.207540461 | 0.207540461 | loss | 0.524763043 | 0.317222582 | | 37 | 5 |
| cg24345184 | 1.30E−09 | 5.52E−04 | −0.20752259 | 0.20752259 | loss | 0.495883008 | 0.288360418 | TBX1; TBX1; TBX1 | 37 | 22 |
| cg03866211 | 1.01E−09 | 4.27E−04 | −0.207509255 | 0.207509255 | loss | 0.836309143 | 0.628799888 | ALOX5; ALOX5; ALOX5 | 37 | 10 |
| cg23349301 | 7.13E−10 | 3.03E−04 | −0.207505891 | 0.207505891 | loss | 0.549322674 | 0.341816782 | | 37 | 17 |
| cg13679679 | 2.78E−09 | 1.18E−03 | −0.207490927 | 0.207490927 | loss | 0.621845357 | 0.414354429 | CMC1 | 37 | 3 |
| cg07674022 | 1.01E−09 | 4.27E−04 | −0.207490403 | 0.207490403 | loss | 0.795549426 | 0.588059024 | TRPC3; TRPC3; TRPC3 | 37 | 4 |
| cg16763089 | 3.28E−09 | 1.39E−03 | −0.20748429 | 0.20748429 | loss | 0.33331146 | 0.125827171 | LINC00654; LINC00654 | 37 | 20 |
| cg09318162 | 1.83E−09 | 7.75E−04 | −0.207481727 | 0.207481727 | loss | 0.675107062 | 0.467625335 | | 37 | 6 |
| cg01987516 | 1.30E−09 | 5.52E−04 | −0.207473954 | 0.207473954 | loss | 0.840108525 | 0.632634571 | | 37 | 10 |
| cg03981535 | 1.19E−09 | 5.07E−04 | −0.207471995 | 0.207471995 | loss | 0.518317266 | 0.310845271 | RNPEP | 37 | 1 |
| cg24148085 | 7.13E−10 | 3.03E−04 | −0.20747072 | 0.20747072 | loss | 0.569855143 | 0.362384424 | | 37 | 3 |
| cg16317457 | 1.83E−09 | 7.75E−04 | −0.207458604 | 0.207458604 | loss | 0.513544245 | 0.306085641 | | 37 | 11 |
| cg07447742 | 1.30E−09 | 5.52E−04 | −0.207453219 | 0.207453219 | loss | 0.689025508 | 0.481572288 | MLNR | 37 | 13 |
| cg15383087 | 7.13E−10 | 3.03E−04 | −0.207433566 | 0.207433566 | loss | 0.759693466 | 0.5522599 | ZFP82 | 37 | 19 |
| cg00628788 | 1.19E−09 | 5.07E−04 | −0.207431405 | 0.207431405 | loss | 0.433872064 | 0.226440659 | | 37 | 18 |
| cg05497750 | 5.85E−09 | 2.49E−03 | −0.207406495 | 0.207406495 | loss | 0.671626319 | 0.464219824 | CHP2 | 37 | 16 |
| cg19092396 | 7.48E−09 | 3.18E−03 | −0.207405229 | 0.207405229 | loss | 0.458969015 | 0.251563786 | | 37 | 1 |
| cg06156640 | 9.54E−09 | 4.05E−03 | −0.207402355 | 0.207402355 | loss | 0.856057796 | 0.648655441 | ALLC | 37 | 2 |
| cg02309230 | 1.68E−09 | 7.12E−04 | −0.207380503 | 0.207380503 | loss | 0.763077062 | 0.555696559 | | 37 | 6 |
| cg11923441 | 7.13E−10 | 3.03E−04 | −0.207376282 | 0.207376282 | loss | 0.6346703 | 0.427294018 | | 37 | 8 |
| cg16348254 | 7.77E−10 | 3.30E−04 | −0.207375963 | 0.207375963 | loss | 0.84352194 | 0.636145976 | | 37 | 10 |
| cg07981328 | 1.03E−08 | 4.39E−03 | −0.207368222 | 0.207368222 | loss | 0.468460236 | 0.261092014 | | 37 | 1 |
| cg01084740 | 8.47E−10 | 3.60E−04 | −0.207360503 | 0.207360503 | loss | 0.518967332 | 0.311606829 | TMEM30B | 37 | 14 |
| cg10645091 | 4.64E−08 | 1.97E−02 | −0.207350361 | 0.207350361 | loss | 0.792488685 | 0.585138324 | C13orf45 | 37 | 13 |
| cg01812577 | 7.13E−10 | 3.03E−04 | −0.207343193 | 0.207343193 | loss | 0.452924587 | 0.245581394 | CHRM1; CHRM1 | 37 | 11 |
| cg16346321 | 7.13E−10 | 3.03E−04 | −0.207334896 | 0.207334896 | loss | 0.798329096 | 0.5909942 | CASZ1; CASZ1 | 37 | 1 |
| cg12673429 | 9.23E−10 | 3.92E−04 | −0.207321414 | 0.207321414 | loss | 0.429282002 | 0.221960588 | PIEZO2 | 37 | 18 |
| cg05566455 | 1.99E−09 | 8.43E−04 | −0.207303368 | 0.207303368 | loss | 0.533356874 | 0.326053506 | KCNJ5; C11orf45; C11orf45; | 37 | 11 |
| | | | | | | | | C11orf45 | | |
| cg00123104 | 7.13E−10 | 3.03E−04 | −0.207280004 | 0.207280004 | loss | 0.351330357 | 0.144050353 | CLSTN1; CLSTN1; CLSTN1; | 37 | 1 |
| | | | | | | | | CLSTN1 | | |
| cg11334406 | 1.99E−09 | 8.43E−04 | −0.207271697 | 0.207271597 | loss | 0.747275726 | 0.540004029 | ALOX15 | 37 | 17 |
| cg05180451 | 7.48E−09 | 3.18E−03 | −0.207267919 | 0.207267919 | loss | 0.68068346 | 0.473415541 | | 37 | 6 |
| cg23312397 | 1.42E−09 | 6.01E−04 | −0.207247156 | 0.207247156 | loss | 0.557037221 | 0.349790065 | BDKRB2 | 37 | 14 |
| cg18922062 | 7.77E−10 | 3.30E−04 | −0.207241044 | 0.207241044 | loss | 0.717488785 | 0.510247741 | BTNL9 | 37 | 5 |
| cg12072164 | 3.88E−09 | 1.65E−03 | −0.207230784 | 0.207230784 | loss | 0.603417519 | 0.396186735 | LYPD5; LYPD5; LYPD5 | 37 | 19 |
| cg12155036 | 7.13E−10 | 3.03E−04 | −0.207230107 | 0.207230107 | loss | 0.570090013 | 0.362859906 | | 37 | 3 |
| cg25220063 | 7.13E−10 | 3.03E−04 | −0.207204371 | 0.207204371 | loss | 0.27123633 | 0.054031959 | | 37 | 1 |
| cg00056257 | 1.54E−09 | 6.54E−04 | −0.207155548 | 0.207155548 | loss | 0.68602573 | 0.478870182 | GRM7; GRM7 | 37 | 3 |
| cg17093267 | 1.01E−09 | 4.27E−04 | −0.207143471 | 0.207143471 | loss | 0.51072793 | 0.303584459 | XPNPEP1; XPNPEP1; XPNPEP1 | 37 | 10 |
| cg06153927 | 7.13E−10 | 3.03E−04 | −0.207127386 | 0.207127386 | loss | 0.820879268 | 0.613751882 | | 37 | 6 |
| cg10685359 | 6.80E−08 | 2.89E−02 | −0.207094408 | 0.207094408 | loss | 0.495968496 | 0.288874088 | C4orf36 | 37 | 4 |
| cg17620300 | 6.89E−09 | 2.93E−03 | −0.207049177 | 0.207049177 | loss | 0.804841925 | 0.597792747 | | 37 | 6 |
| cg21864304 | 1.07E−07 | 4.55E−02 | −0.207045483 | 0.207045483 | loss | 0.862694936 | 0.655649453 | VGLL4; VGLL4 | 37 | 3 |
| cg18637459 | 8.47E−10 | 3.60E−04 | −0.207037579 | 0.207037579 | loss | 0.353071194 | 0.146033615 | TMEM119; TMEM119 | 37 | 12 |
| cg07469063 | 1.10E−09 | 4.65E−04 | −0.207033466 | 0.207033466 | loss | 0.336543298 | 0.129509832 | | 37 | 15 |
| cg23513363 | 7.13E−10 | 3.03E−04 | −0.207017418 | 0.207017418 | loss | 0.622357189 | 0.415339771 | | 37 | 10 |
| cg17425810 | 8.56E−10 | 3.63E−04 | −0.206993248 | 0.205993248 | loss | 0.39860186 | 0.191608612 | TRABD2B | 37 | 1 |
| cg11251135 | 7.13E−10 | 3.03E−04 | −0.206980252 | 0.206980252 | loss | 0.923541328 | 0.716561076 | RTN4RL1 | 37 | 17 |
| cg11414913 | 1.54E−09 | 6.54E−04 | −0.206961165 | 0.206961165 | loss | 0.764735153 | 0.557773988 | | 37 | 1 |
| cg15050310 | 4.21E−09 | 1.79E−03 | −0.206959504 | 0.206959504 | loss | 0.874105245 | 0.667145741 | | 37 | 11 |
| cg00500770 | 7.77E−10 | 3.30E−04 | −0.206933519 | 0.206933619 | loss | 0.76890236 | 0.561968741 | | 37 | 20 |
| cg08530537 | 7.13E−10 | 3.03E−04 | −0.206898109 | 0.206898109 | loss | 0.702836468 | 0.495938359 | DPEP3; DPEP3; DPEP3; DPEP3 | 37 | 16 |
| cg09382492 | 8.47E−10 | 3.60E−04 | −0.206819503 | 0.206819503 | loss | 0.726495526 | 0.519676024 | AANAT; AANAT | 37 | 17 |
| cg15822328 | 2.06E−09 | 8.75E−04 | −0.206809149 | 0.206809149 | loss | 0.578566649 | 0.3717575 | LOC254099 | 37 | 1 |
| cg04829830 | 1.12E−08 | 4.76E−03 | −0.206804164 | 0.206804164 | loss | 0.718654575 | 0.511850412 | CEP170B; CEP170B | 37 | 14 |
| cg07421287 | 7.13E−10 | 3.03E−04 | −0.206769413 | 0.206769413 | loss | 0.542159325 | 0.335389912 | KCNA3 | 37 | 1 |
| cg24439334 | 7.13E−10 | 3.03E−04 | −0.206766306 | 0.206766306 | loss | 0.858774306 | 0.652008 | PEX10; PEX10 | 37 | 1 |
| cg06376715 | 7.13E−10 | 3.03E−04 | −0.206756758 | 0.206756758 | loss | 0.462732081 | 0.255975324 | TP73; TP73; TP73; TP73; TP73; | 37 | 1 |
| | | | | | | | | TP73; TP73; TP73; TP73; | | |
| | | | | | | | | TP73; TP73; TP73; TP73 | | |
| cg10941424 | 8.55E−05 | 3.63E−02 | −0.20672612 | 0.20672612 | loss | 0.897450696 | 0.690724576 | | 37 | 12 |
| cg03652339 | 7.13E−10 | 3.03E−04 | −0.205703239 | 0.206703239 | loss | 0.433998798 | 0.277295559 | DUSPS | 37 | 10 |
| cg17207736 | 5.85E−09 | 2.49E−03 | −0.205699264 | 0.206699264 | loss | 0.576920717 | 0.370221453 | SLC45A4; SLC45A4; SLC45A4 | 37 | 8 |
| cg00599163 | 1.81E−08 | 7.69E−03 | −0.20667994 | 0.20667994 | loss | 0.740818823 | 0.534138882 | | 37 | 2 |
| cg02757179 | 7.13E−10 | 3.03E−04 | −0.206679695 | 0.206679695 | loss | 0.877601877 | 0.670922182 | POMC; POMC | 37 | 2 |
| cg25343227 | 7.13E−10 | 3.03E−04 | −0.206673875 | 0.206673875 | loss | 0.619952798 | 0.413278924 | LINC00634 | 37 | 22 |
| cg14095959 | 7.13E−10 | 3.03E−04 | −0.205618181 | 0.206618181 | loss | 0.38301574 | 0.176397559 | | 37 | 4 |
| cg22234712 | 8.80E−09 | 3.74E−03 | −0.20659948 | 0.20659948 | loss | 0.331173758 | 0.124574279 | MIR3150B; MIR3150B | 37 | 8 |
| cg10081469 | 2.78E−09 | 1.18E−03 | −0.205588393 | 0.206588393 | loss | 0.470271317 | 0.263682924 | TMEM30B; TMEM30B | 37 | 14 |
| cg04777352 | 2.35E−09 | 9.98E−04 | −0.206585657 | 0.206585657 | loss | 0.749060857 | 0.5424752 | | 37 | 8 |
| cg08889843 | 7.13E−10 | 3.03E−04 | −0.206579778 | 0.206579778 | loss | 0.562083719 | 0.355503941 | SBK2; SBK2 | 37 | 19 |
| cg20925811 | 7.13E−10 | 3.03E−04 | −0.206576477 | 0.206575477 | loss | 0.719210589 | 0.512634112 | MMP9 | 37 | 20 |
| cg03364381 | 9.54E−09 | 4.05E−03 | −0.206575672 | 0.206575672 | loss | 0.47639545 | 0.269819788 | LINC00111 | 37 | 21 |
| cg11684572 | 7.13E−10 | 3.03E−04 | −0.206567234 | 0.206567234 | loss | 0.82068364 | 0.614116406 | DCA2 | 37 | 15 |
| cg20980321 | 7.13E−10 | 3.03E−04 | −0.206563638 | 0.206563638 | loss | 0.436461679 | 0.229898041 | AGPAT1; RNF5; AGPAT1 | 37 | 6 |
| cg11549848 | 1.03E−08 | 4.39E−03 | −0.206552368 | 0.206562368 | loss | 0.879496426 | 0.672934059 | C1QTNF9B- | 37 | 13 |
| | | | | | | | | AS1; C1QTNF9B- | | |
| | | | | | | | | AS1; MIPEP | | |
| cg02713669 | 1.68E−09 | 7.12E−04 | −0.206550156 | 0.206550156 | loss | 0.274137988 | 0.067587832 | SH3RF3; SH3Rf3- | 37 | 2 |
| | | | | | | | | AS1; SH3RF3-AS1 | | |
| cg10551185 | 1.01E−09 | 4.27E−04 | 0.206536143 | 0.205536143 | loss | 0.806324202 | 0.599788059 | | 37 | 5 |
| cg00397764 | 1.01E−09 | 4.27E−04 | −0.206512183 | 0.206512183 | loss | 0.758952094 | 0.552439912 | TDRD1 | 37 | 10 |
| cg10672681 | 8.47E−10 | 3.60E−04 | −0.206495343 | 0.206495343 | loss | 0.658458343 | 0.451963 | ABHD10; ABHD10; ABHD10; | 37 | 3 |
| | | | | | | | | ABHD10 | | |
| cg24003560 | 7.13E−10 | 3.03E−04 | −0.206491069 | 0.206491069 | loss | 0.387798228 | 0.181307159 | C1orf115 | 37 | 1 |
| cg22748407 | 7.13E−10 | 3.03E−04 | −0.206464833 | 0.206464833 | loss | 0.430910968 | 0.224446135 | USP22 | 37 | 17 |
| cg02481789 | 7.13E−10 | 3.03E−04 | −0.206454089 | 0.206454089 | loss | 0.476056572 | 0.269602482 | | 37 | 17 |
| cg06819763 | 7.77E−10 | 3.30E−04 | −0.206429276 | 0.206429276 | loss | 0.262707204 | 0.056277928 | | 37 | 2 |
| cg25940248 | 1.54E−09 | 6.54E−04 | −0.206425215 | 0.206425215 | loss | 0.566170409 | 0.359745194 | KLB | 37 | 4 |
| cg25925441 | 1.30E−09 | 5.52E−04 | −0.206391818 | 0.206391818 | loss | 0.657066889 | 0.450675071 | RPRM | 37 | 2 |
| cg15600238 | 7.13E−10 | 3.03E−04 | −0.206389833 | 0.206389833 | loss | 0.778705021 | 0.572315188 | | 37 | 7 |
| cg14221644 | 7.84E−10 | 3.33E−04 | −0.206387808 | 0.206387808 | loss | 0.664556485 | 0.458168676 | | 37 | 9 |
| cg20652899 | 1.30E−09 | 5.52E−04 | −0.206372337 | 0.206372337 | loss | 0.699496896 | 0.493124559 | FXYD6; FXYD6; FXYD6; FXYD6; | 37 | 11 |
| | | | | | | | | FXYD6; FXYD6; FXYD6; | | |
| | | | | | | | | FXYD6-FXYD6; FXYD6- | | |
| | | | | | | | | FXYD6; FXYD6- | | |
| | | | | | | | | FXYD6; FXYD6- | | |
| | | | | | | | | FXYD6; FXYD6; FXYD6 | | |
| cg01076229 | 1.30E−09 | 5.52E−04 | −0.206358902 | 0.206358902 | loss | 0.723380255 | 0.517021353 | CRISP2; CRISP2; CRISP2; CRISP2; | 37 | 6 |
| | | | | | | | | CRISP2; CRISP2; CRISP2; | | |
| | | | | | | | | CRISP2; CRISP2; CRISP2; | | |
| | | | | | | | | CRISP2; CRISP2 | | |
| cg00174500 | 7.13E−10 | 3.03E−04 | −0.206358369 | 0.206358369 | loss | 0.781855681 | 0.575497312 | CMTMS; CMTMS | 37 | 14 |
| cg04546557 | 1.10E−09 | 4.65E−04 | −0.206355331 | 0.206355331 | loss | 0.704895555 | 0.498540224 | | 37 | 7 |
| cg05601974 | 7.13E−10 | 3.03E−04 | −0.206350969 | 0.206350969 | loss | 0.57749554 | 0.371144571 | SMARCD3; SMARCD3:SMARCD3 | 37 | 7 |
| cg23575603 | 7.13E−10 | 3.03E−04 | −0.206315773 | 0.206315773 | loss | 0.686598179 | 0.480282406 | | 37 | 13 |
| cg23989012 | 1.01E−09 | 4.27E−04 | −0.206288406 | 0.206288406 | loss | 0.541975558 | 0.335687153 | NBPF8; NBPF8; NBPF8 | 37 | 1 |
| cg19274341 | 9.27E−08 | 3.94E−02 | −0.206283801 | 0.206283801 | loss | 0.35868325 | 0.152399449 | | 37 | 1 |
| cg25112202 | 8.47E−10 | 3.60E−04 | −0.206256304 | 0.206256304 | loss | 0.389700104 | 0.1834438 | | 37 | 3 |
| cg20806180 | 9.23E−10 | 3.92E−04 | −0.206255412 | 0.206255412 | loss | 0.936648189 | 0.730392776 | MGAT3 | 37 | 22 |
| cg26230651 | 7.13E−10 | 3.03E−04 | −0.206248537 | 0.206248537 | loss | 0.282704055 | 0.076455518 | | 37 | 2 |
| cg19208681 | 7.13E−10 | 3.03E−04 | −0.206230978 | 0.206230978 | loss | 0.759304749 | 0.553073771 | TEX14; RAD51C; TEX14; RAD51C; | 37 | 17 |
| | | | | | | | | TEX14; RAD51C; RAD51C | | |
| cg07589899 | 1.54E−09 | 6.54E−04 | −0.20622585 | 0.20622585 | loss | 0.379671113 | 0.173445264 | | 37 | 2 |
| cg16596957 | 7.77E−10 | 3.30E−04 | −0.206186551 | 0.206186551 | loss | 0.671786498 | 0.465599947 | | 37 | 16 |
| cg12984635 | 1.83E−09 | 7.75E−04 | −0.206175196 | 0.206175196 | loss | 0.762599213 | 0.556424018 | ETHE1 | 37 | 19 |
| cg21004358 | 3.02E−09 | 1.28E−03 | −0.206144953 | 0.206144953 | loss | 0.811124458 | 0.604979506 | F11-AS1; F11-AS1; F11- | 37 | 4 |
| | | | | | | | | F11-AS1 | | |
| cg07444554 | 7.77E−10 | 3.30E−04 | −0.206129722 | 0.206129722 | loss | 0.858453134 | 0.652323412 | NRG2; NRG2; NRG2; NRG2; | 37 | 5 |
| | | | | | | | | NRG2 | | |
| cg11566975 | 7.13E−10 | 3.03E−04 | −0.206129088 | 0.206129088 | loss | 0.322287923 | 0.116158835 | LAMB2 | 37 | 3 |
| cg08030082 | 7.13E−10 | 3.03E−04 | −0.206101586 | 0.206101586 | loss | 0.512738798 | 0.306637212 | POMC; POMC | 37 | 2 |
| cg16817668 | 7.13E−10 | 3.03E−04 | −0.206099118 | 0.206099118 | loss | 0.953487553 | 0.747388435 | | 37 | 1 |
| cg18180846 | 7.13E−10 | 3.03E−04 | −0.206094292 | 0.206094292 | loss | 0.737342115 | 0.531247824 | LBX1-AS1 | 37 | 10 |
| cg03464655 | 1.16E−07 | 4.91E−02 | −0.206074368 | 0.206074368 | loss | 0.356127815 | 0.150053447 | SLC38A11; SLC38A11; SLC38A11; | 37 | 2 |
| | | | | | | | | SLC38A11 | | |
| cg05812599 | 7.13E−10 | 3.03E−04 | −0.20605712 | 0.20605712 | loss | 0.914849643 | 0.708792524 | CLPB; CLPB; CLP8; CLPB | 37 | 11 |
| cg15865892 | 2.78E−09 | 1.18E−03 | −0.20605344 | 0.20605344 | loss | 0.746113034 | 0.540059594 | | 37 | 2 |
| cg09571848 | 7.13E−10 | 3.03E−04 | −0.206052829 | 0.206052829 | loss | 0.908400711 | 0.702347882 | | 37 | 5 |
| cg03366850 | 9.23E−10 | 3.92E−04 | −0.206039059 | 0.206039059 | loss | 0.50711857 | 0.301079511 | TRIL | 37 | 7 |
| cg05754122 | 7.13E−10 | 3.03E−04 | −0.206027584 | 0.206027584 | loss | 0.661131996 | 0.455104412 | TMPRSS6; TMPRSS6 | 37 | 22 |
| cg00255882 | 3.88E−09 | 1.65E−03 | −0.206014256 | 0.206014256 | loss | 0.283361243 | 0.077346987 | WDR33; WDR33; WDR33 | 37 | 2 |
| cg00237876 | 7.77E−10 | 3.30E−04 | −0.205999366 | 0.205999366 | loss | 0.763743308 | 0.557743941 | CD300LG; CD300LG; CD300LG; | 37 | 17 |
| | | | | | | | | CD300LG | | |
| cg24479859 | 2.16E−09 | 9.17E−04 | −0.205978074 | 0.205978074 | loss | 0.895284809 | 0.689306735 | | 37 | 13 |
| cg15571353 | 8.47E−10 | 3.60E−04 | −0.205973668 | 0.205973668 | loss | 0.750883204 | 0.544909535 | LOC284632 | 37 | 1 |
| cg10647465 | 4.29E−08 | 1.82E−02 | 0.205968382 | 0.205968382 | gain | 0.387666042 | 0.593634424 | KIRREL3; KIRREL3 | 37 | 11 |
| cg09012594 | 1.89E−09 | 8.02E−04 | −0.205959496 | 0.205959496 | loss | 0.445125215 | 0.239165719 | NEBL; NEBL | 37 | 10 |
| cg15263021 | 7.13E−10 | 3.03E−04 | −0.205944496 | 0.205944496 | loss | 0.437881902 | 0.231937406 | | 37 | 11 |
| cg00761968 | 7.77E−10 | 3.30E−04 | −0.205915631 | 0.205915631 | loss | 0.618825272 | 0.412909641 | LECT1; LECT1; LECT1; LECT1 | 37 | 13 |
| cg04436389 | 7.13E−10 | 3.03E−04 | −0.205896806 | 0.205896806 | loss | 0.786103947 | 0.580207141 | NANOS2 | 37 | 19 |
| cg16550453 | 6.89E−09 | 2.93E−03 | −0.205843306 | 0.205843306 | loss | 0.9672474 | 0.761404094 | TDRD1 | 37 | 10 |
| cg10184431 | 7.13E−10 | 3.03E−04 | −0.205830799 | 0.205830799 | loss | 0.362697475 | 0.156866676 | HIST1HZBM; HIST1H2AJ | 37 | 6 |
| cg03960424 | 7.13E−10 | 3.03E−04 | −0.205817639 | 0.205817639 | loss | 0.466271709 | 0.260454071 | APCDD1L | 37 | 20 |
| cg12945707 | 8.47E−10 | 3.60E−04 | −0.205816686 | 0.205816686 | loss | 0.695054257 | 0.489237571 | COL11A2; COL11A2; COL11A2 | 37 | 6 |
| cg13460850 | 8.11E−09 | 3.44E−03 | −0.205798084 | 0.205798084 | loss | 0.795238966 | 0.589440882 | | 37 | 3 |
| cg26717563 | 1.01E−09 | 4.27E−04 | −0.205793438 | 0.205793438 | loss | 0.777349485 | 0.571556047 | TLE2; TLE2; TLE2 | 37 | 19 |
| cg10978034 | 1.30E−09 | 5.52E−04 | −0.205791372 | 0.205791372 | loss | 0.879886672 | 0.6740953 | | 37 | 6 |
| cg04938738 | 7.13E−10 | 3.03E−04 | −0.205783064 | 0.205783064 | loss | 0.557347858 | 0.351564794 | MOB2 | 37 | 11 |
| cg19645255 | 7.13E−10 | 3.03E−04 | −0.205761271 | 0.205761271 | loss | 0.583899494 | 0.378138224 | TUSC5 | 37 | 17 |
| cg21219373 | 3.83E−09 | 1.62E−03 | −0.20574391 | 0.20574391 | loss | 0.943457154 | 0.737713244 | PPP1R13L; PPP1R13L | 37 | 19 |
| cg06761424 | 1.67E−08 | 7.10E−03 | −0.205707824 | 0.205707824 | loss | 0.59297063 | 0.387262806 | KIAA1210 | 37 | X |
| cg27006947 | 8.64E−08 | 3.67E−02 | −0.205706855 | 0.205706855 | loss | 0.464201121 | 0.258494266 | | 37 | 7 |
| cg06888406 | 2.56E−09 | 1.09E−03 | −0.2056895 | 0.2056895 | loss | 0.890659594 | 0.684970094 | | 37 | 10 |
| cg01784928 | 8.47E−10 | 3.60E−04 | −0.205680744 | 0.205680744 | loss | 0.699292085 | 0.493611341 | TMEM63C | 37 | 14 |
| cg26986681 | 7.92E−08 | 3.36E−02 | −0.205648945 | 0.205648945 | loss | 0.299581707 | 0.093932761 | IGFBP7-AS1 | 37 | 4 |
| cg02683408 | 7.13E−10 | 3.03E−04 | −0.20562034 | 0.20562034 | loss | 0.891971575 | 0.686351235 | | 37 | 17 |
| cg03680291 | 5.41E−08 | 2.30E−02 | −0.205592736 | 0.205592736 | loss | 0.557943836 | 0.3523511 | | 37 | 16 |
| cg00003578 | 7.13E−10 | 3.03E−04 | −0.205589422 | 0.205589422 | loss | 0.58490314 | 0.379313718 | | 37 | 1 |
| cg13779349 | 6.89E−09 | 2.93E−03 | −0.205551035 | 0.205551035 | loss | 0.50399147 | 0.298440435 | ZCCHC16; TRPC5 | 37 | X |
| cg04944300 | 7.77E−10 | 3.30E−04 | −0.205532594 | 0.205532594 | loss | 0.844013994 | 0.6384514 | ESPNP | 37 | 1 |
| cg02838492 | 7.13E−10 | 3.03E−04 | −0.20552946 | 0.20552946 | loss | 0.662624808 | 0.457095347 | KIF12; KIF12 | 37 | 9 |
| cg21829490 | 8.80E−09 | 3.74E−03 | −0.205492015 | 0.205492015 | loss | 0.535207945 | 0.329715931 | | 37 | 18 |
| cg25228169 | 7.13E−10 | 3.03E−04 | −0.205484225 | 0.205484225 | loss | 0.794047572 | 0.588563347 | GRAMD2 | 37 | 15 |
| cg24790788 | 2.56E−09 | 1.09E−03 | −0.205472179 | 0.205472179 | loss | 0.507495826 | 0.302023647 | PLOD2; PLOD2 | 37 | 3 |
| cg03931865 | 1.99E−09 | 8.43E−04 | −0.205464239 | 0.205464239 | loss | 0.685345351 | 0.479881112 | | 37 | 16 |
| cg09669835 | 7.13E−10 | 3.03E−04 | −0.205462235 | 0.205462235 | loss | 0.359501732 | 0.154039497 | MTERF | 37 | 7 |
| cg11968804 | 7.13E−10 | 3.03E−04 | −0.205434421 | 0.205434421 | loss | 0.309019462 | 0.103585041 | | 37 | 1 |
| cg21456083 | 7.13E−10 | 3.03E−04 | −0.205415066 | 0.205415066 | loss | 0.33480856 | 0.129393494 | | 37 | 19 |
| cg24778776 | 8.11E−09 | 3.44E−03 | −0.20540868 | 0.20540868 | loss | 0.879495362 | 0.674086682 | | 37 | 11 |
| cg07745707 | 7.13E−10 | 3.03E−04 | −0.205407566 | 0.205407566 | loss | 0.802264625 | 0.596857059 | ZNF469 | 37 | 16 |
| cg22194188 | 7.13E−10 | 3.03E−04 | −0.205406167 | 0.205406167 | loss | 0.375267306 | 0.169861139 | TEX2 | 37 | 17 |
| cg14884373 | 7.13E−10 | 3.03E−04 | −0.205402413 | 0.205402413 | loss | 0.733392508 | 0.527990094 | GFRA4; GFRA4; GFRA4; GFRA4 | 37 | 20 |
| cg14621323 | 7.13E−10 | 3.03E−04 | −0.205397583 | 0.205397583 | loss | 0.877531577 | 0.672133994 | PFN3 | 37 | 5 |
| cg17924225 | 7.13E−10 | 3.03E−04 | −0.205364486 | 0.205364486 | loss | 0.850011804 | 0.644647318 | | 37 | 5 |
| cg02680566 | 7.13E−10 | 3.03E−04 | −0.205353095 | 0.205353095 | loss | 0.664453325 | 0.459100229 | OSR1 | 37 | 2 |
| cg14560430 | 1.12E−08 | 4.76E−03 | −0.205337587 | 0.205337587 | loss | 0.73452107 | 0.529183482 | TRIM71 | 37 | 3 |
| cg20254361 | 7.13E−10 | 3.03E−04 | −0.20532842 | 0.20532842 | loss | 0.691602832 | 0.486274412 | MYOM3; MYOM3 | 37 | 1 |
| cg00068428 | 1.54E−09 | 6.54E−04 | −0.205308487 | 0.205308487 | loss | 0.67936924 | 0.474060753 | | 37 | 8 |
| cg05554161 | 7.77E−10 | 3.30E−04 | −0.20530392 | 0.20530392 | loss | 0.598127738 | 0.392823818 | OLFML2A | 37 | 9 |
| cg07655487 | 1.01E−09 | 4.27E−04 | −0.205302801 | 0.205302801 | loss | 0.386478113 | 0.181175312 | | 37 | 11 |
| cg02147681 | 2.35E−09 | 9.98E−04 | −0.205277209 | 0.205277209 | loss | 0.509780762 | 0.304503553 | RAI1 | 37 | 17 |
| cg23362884 | 7.13E−10 | 3.03E−04 | −0.205269899 | 0.205269899 | loss | 0.454000217 | 0.248730318 | RBM20 | 37 | 10 |
| cg25181043 | 2.78E−09 | 1.18E−03 | −0.205264368 | 0.205264368 | loss | 0.878238862 | 0.672974494 | | 37 | 20 |
| cg18669381 | 7.77E−10 | 3.30E−04 | −0.205255435 | 0.205255435 | loss | 0.666239553 | 0.460984118 | ARHGEF19; ARHGEF19 | 37 | 1 |
| cg17839399 | 7.13E−10 | 3.03E−04 | −0.205246643 | 0.205246643 | loss | 0.912787049 | 0.707540406 | ETNK1; ETNK1 | 37 | 12 |
| cg24136671 | 7.13E−10 | 3.03E−04 | −0.205245873 | 0.205245873 | loss | 0.851142479 | 0.645896606 | | 37 | 14 |
| cg16270485 | 7.13E−10 | 3.03E−04 | −0.205237819 | 0.205237819 | loss | 0.342610208 | 0.137372388 | ALPL; ALPL; ALPL | 37 | 1 |
| cg00670942 | 1.19E−09 | 5.07E−04 | −0.205231512 | 0.205231512 | loss | 0.413700889 | 0.208469376 | | 37 | 17 |
| cg21243717 | 7.13E−10 | 3.03E−04 | −0.20521893 | 0.20521893 | loss | 0.820451947 | 0.615233018 | | 37 | 5 |
| cg09759994 | 8.47E−10 | 3.60E−04 | −0.205217746 | 0.205217746 | loss | 0.346164625 | 0.140946878 | TTL | 37 | 2 |
| cg04353586 | 2.16E−09 | 9.17E−04 | −0.205212991 | 0.205212991 | loss | 0.874929221 | 0.669716229 | CAMTA1 | 37 | 1 |
| cg16412370 | 7.13E−10 | 3.03E−04 | −0.205212331 | 0.205212331 | loss | 0.824062049 | 0.618849718 | HSF2BP; RRP1B | 37 | 21 |
| cg25875537 | 7.13E−10 | 3.03E−04 | −0.20520395 | 0.20520395 | loss | 0.442714826 | 0.237510876 | | 37 | 8 |
| cg03575765 | 9.23E−10 | 3.92E−04 | −0.205201815 | 0.205201815 | loss | 0.642750521 | 0.437548706 | | 37 | 14 |
| cg06719448 | 5.84E−08 | 2.48E−02 | −0.205172571 | 0.205172571 | loss | 0.727463847 | 0.522291276 | FAM71F1; FAM71F1; FAM71F1; | 37 | 7 |
| | | | | | | | | FAM71F1; FAM71F1 | | |
| cg10724867 | 7.13E−10 | 3.03E−04 | −0.205169232 | 0.205169232 | loss | 0.841875832 | 0.6367066 | HOXA10; HOXA10-HOXA9 | 37 | 7 |
| cg24166966 | 7.13E−10 | 3.03E−04 | −0.205112853 | 0.205112853 | loss | 0.344272234 | 0.139159381 | FBXL2; FBXL2 | 37 | 3 |
| cg13710556 | 7.13E−10 | 3.03E−04 | −0.205109615 | 0.205109615 | loss | 0.490289021 | 0.285179406 | | 37 | 7 |
| cg24550343 | 7.13E−10 | 3.03E−04 | −0.205096801 | 0.205096801 | loss | 0.882989019 | 0.677892218 | | 37 | 5 |
| cg09732145 | 7.13E−10 | 3.03E−04 | −0.205083843 | 0.205083843 | loss | 0.571591155 | 0.366507312 | | 37 | 3 |
| cg23369909 | 1.10E−09 | 4.65E−04 | −0.205077319 | 0.205077319 | loss | 0.704075777 | 0.498998459 | | 37 | 11 |
| cg17828057 | 2.48E−08 | 1.05E−02 | −0.205074668 | 0.205074668 | loss | 0.539889204 | 0.334814535 | PLAT; PLAT | 37 | 8 |
| cg03480346 | 3.88E−09 | 1.65E−03 | −0.20506447 | 0.20506447 | loss | 0.64986467 | 0.4448002 | MFSD2B | 37 | 2 |
| cg07915343 | 1.68E−09 | 7.12E−04 | −0.205056127 | 0.205056127 | loss | 0.743124868 | 0.538068741 | LDHAL6A; LDHAL6A | 37 | 11 |
| cg10831901 | 2.35E−09 | 9.98E−04 | −0.205031968 | 0.205031968 | loss | 0.294094992 | 0.089063024 | LINC00654; LINC00654 | 37 | 20 |
| cg18438319 | 7.13E−10 | 3.03E−04 | −0.2050014 | 0.2050014 | loss | 0.480304511 | 0.275303112 | | 37 | 7 |
| cg22200736 | 7.13E−10 | 3.03E−04 | −0.204998767 | 0.204998767 | loss | 0.466990049 | 0.261991282 | KRT72; KRT72; KRT72; KRT72; | 37 | 12 |
| | | | | | | | | KRT72; KRT72 | | |
| cg14809332 | 2.78E−09 | 1.18E−03 | −0.204995993 | 0.204995993 | loss | 0.547201187 | 0.342205194 | MROH6; MROH6 | 37 | 8 |
| cg26034481 | 8.47E−10 | 3.60E−04 | −0.204995798 | 0.204995798 | loss | 0.806646645 | 0.601650847 | | 37 | 14 |
| cg09867953 | 7.13E−10 | 3.03E−04 | −0.204987083 | 0.204987083 | loss | 0.489220377 | 0.284233294 | ERN2 | 37 | 16 |
| cg21695020 | 7.13E−10 | 3.03E−04 | −0.204978214 | 0.204978214 | loss | 0.839271432 | 0.634293218 | DPEP3; DPEP3; DPEP3; DPEPS | 37 | 16 |
| cg02395590 | 7.13E−10 | 3.03E−04 | −0.204976904 | 0.204976904 | loss | 0.834250351 | 0.629273447 | | 37 | 5 |
| cg16731266 | 8.47E−10 | 3.60E−04 | −0.204973634 | 0.204973634 | loss | 0.81390104 | 0.608927406 | | 37 | 6 |
| cg27629589 | 9.23E−10 | 3.92E−04 | −0.2049395 | 0.2049395 | loss | 0.752515647 | 0.547576147 | | 37 | 2 |
| cg02109484 | 7.13E−10 | 3.03E−04 | −0.204923779 | 0.204923779 | loss | 0.309180589 | 0.104256809 | M1AP; M1AP; M1AP; M1AP | 37 | 2 |
| cg05191217 | 7.77E−10 | 3.30E−04 | −0.204907753 | 0.204907753 | loss | 0.976246547 | 0.771338794 | | 37 | 5 |
| cg04166411 | 1.54E−09 | 6.54E−04 | −0.204896331 | 0.204896331 | loss | 0.755063619 | 0.550167288 | EBF2 | 37 | 8 |
| cg21377521 | 1.42E−09 | 6.01E−04 | −0.204894124 | 0.204894124 | loss | 0.854332247 | 0.649438124 | TMTC1; TMTC1 | 37 | 12 |
| cg01781963 | 1.68E−09 | 7.12E−04 | −0.204874461 | 0.204874461 | loss | 0.758994891 | 0.554120429 | | 37 | 5 |
| cg20974196 | 7.77E−10 | 3.30E−04 | −0.204865053 | 0.204865053 | loss | 0.405569558 | 0.200704506 | CFD | 37 | 19 |
| cg01435791 | 7.13E−10 | 3.03E−04 | −0.204856748 | 0.204856748 | loss | 0.814614525 | 0.609757776 | | 37 | 19 |
| cg19908485 | 1.83E−09 | 7.75E−04 | 0.204812366 | 0.204812366 | gain | 0.179347287 | 0.384159653 | NBPF23 | 37 | 1 |
| cg11503274 | 7.77E−10 | 3.30E−04 | −0.204802365 | 0.204802365 | loss | 0.250426503 | 0.045624138 | COX1 | 37 | 5 |
| cg12244325 | 7.13E−10 | 3.03E−04 | −0.204778732 | 0.204778732 | loss | 0.811511238 | 0.606732506 | | 37 | 21 |
| cg08013206 | 4.64E−08 | 1.97E−02 | −0.204774703 | 0.204774703 | loss | 0.888128321 | 0.683353618 | | 37 | 7 |
| cg09895590 | 7.13E−10 | 3.03E−04 | −0.204733348 | 0.204733348 | loss | 0.803861277 | 0.599127929 | VAX1 | 37 | 10 |
| cg11395890 | 1.99E−09 | 8.43E−04 | −0.204722923 | 0.204722923 | loss | 0.869035558 | 0.664312635 | FHAD1 | 37 | 1 |
| cg05046097 | 7.13E−10 | 3.03E−04 | −0.204694376 | 0.204694376 | loss | 0.697803553 | 0.493109176 | SNCG; MMRN2 | 37 | 10 |
| cg11248999 | 9.23E−10 | 3.92E−04 | −0.204691948 | 0.204691948 | loss | 0.825423683 | 0.620731735 | EPHB2; EPHB2 | 37 | 1 |
| cg07891179 | 7.13E−10 | 3.03E−04 | −0.204687721 | 0.204687721 | loss | 0.318643457 | 0.113955735 | WIPF3 | 37 | 7 |
| cg07226718 | 7.77E−10 | 3.30E−04 | −0.204679373 | 0.204679373 | loss | 0.786435743 | 0.581756371 | | 37 | 12 |
| cg18060171 | 1.99E−09 | 8.43E−04 | −0.204613766 | 0.204613766 | loss | 0.664982777 | 0.460369012 | CACNA1A; CACNA1A; CACNA1A; | 37 | 19 |
| | | | | | | | | CACNA1A; CACNA1A | | |
| cg14550163 | 7.77E−10 | 3.30E−04 | −0.20459582 | 0.20459582 | loss | 0.880331585 | 0.675735765 | CUX2 | 37 | 12 |
| cg06434451 | 7.13E−10 | 3.03E−04 | −0.204569314 | 0.204569314 | loss | 0.659676832 | 0.455107518 | MARCKSL1; MARCKSL1 | 37 | 1 |
| cg22804000 | 7.13E−10 | 3.03E−04 | −0.204557618 | 0.204557618 | loss | 0.396985247 | 0.192427629 | DFNA5; DFNA5; DFNA5; DFNA5; | 37 | 7 |
| | | | | | | | | DFNA5 | | |
| cg00406022 | 8.80E−09 | 3.74E−03 | −0.204550536 | 0.204550536 | loss | 0.883627683 | 0.679077147 | C6orf25; C6orf25; C6orf25; | 37 | 6 |
| | | | | | | | | C6orf25; C6orf25; C6orf25 | | |
| cg05280814 | 8.47E−10 | 3.60E−04 | −0.204505367 | 0.204505367 | loss | 0.667163002 | 0.462657635 | | 37 | 11 |
| cg05585991 | 7.13E−10 | 3.03E−04 | −0.204475928 | 0.204475928 | loss | 0.86572474 | 0.661248812 | C5orf52 | 37 | 5 |
| cg23300529 | 7.13E−10 | 3.03E−04 | −0.204473044 | 0.204473044 | loss | 0.784812526 | 0.580339482 | HR; HR | 37 | 8 |
| cg09322973 | 1.12E−08 | 4.76E−03 | −0.20447105 | 0.20447105 | loss | 0.319346147 | 0.114875097 | FAM66C | 37 | 12 |
| cg12586386 | 2.12E−08 | 9.01E−03 | −0.204461419 | 0.204461419 | loss | 0.677968825 | 0.473507406 | AQP11 | 37 | 11 |
| cg04826345 | 8.11E−09 | 3.44E−03 | −0.204457914 | 0.204457914 | loss | 0.887117232 | 0.682659318 | | 37 | 13 |
| cg05803853 | 7.13E−10 | 3.03E−04 | −0.20440595 | 0.20440596 | loss | 0.815399325 | 0.610993365 | | 37 | 10 |
| cg09933458 | 2.69E−08 | 1.14E−02 | −0.20440459 | 0.20440459 | loss | 0.298550419 | 0.094145829 | | 37 | 13 |
| cg09256832 | 1.01E−09 | 4.27E−04 | −0.204401982 | 0.204401982 | loss | 0.832675664 | 0.628273682 | SPSB1 | 37 | 1 |
| cg20089744 | 7.13E−10 | 3.03E−04 | −0.204375227 | 0.204375227 | loss | 0.896610009 | 0.692234782 | | 37 | 3 |
| cg16942223 | 7.77E−10 | 3.30E−04 | 0.20436316 | 0.20436316 | gain | 0.230335381 | 0.434698541 | JAKMIP1 | 37 | 4 |
| cg04412755 | 6.35E−09 | 2.70E−03 | −0.204320714 | 0.204320714 | loss | 0.455611091 | 0.251290376 | PTH1R; PTH1R | 37 | 3 |
| cg09042678 | 2.16E−09 | 9.17E−04 | −0.204290723 | 0.204290723 | loss | 0.410201564 | 0.205910841 | PET117 | 37 | 20 |
| cg17969123 | 7.13E−10 | 3.03E−04 | −0.204280165 | 0.204280165 | loss | 0.463984547 | 0.259704382 | | 37 | 19 |
| cg27494647 | 4.29E−08 | 1.82E−02 | −0.20427525 | 0.20427525 | loss | 0.424997306 | 0.220722055 | RARRES2; RARRES2 | 37 | 7 |
| cg04206342 | 2.35E−09 | 9.98E−04 | −0.204246874 | 0.204246874 | loss | 0.535468209 | 0.331221335 | LINC00511 | 37 | 17 |
| cg08019058 | 7.13E−10 | 3.03E−04 | −0.20424549 | 0.20424549 | loss | 0.680115249 | 0.475869759 | | 37 | 2 |
| cg04947136 | 7.13E−10 | 3.03E−04 | −0.204221698 | 0.204221698 | loss | 0.731747857 | 0.527526159 | | 37 | 6 |
| cg05077231 | 7.77E−10 | 3.30E−04 | −0.204216834 | 0.204216834 | loss | 0.661016987 | 0.456800153 | MLNR | 37 | 13 |
| cg13161901 | 1.42E−09 | 6.01E−04 | −0.204158923 | 0.204158923 | loss | 0.627064117 | 0.422905194 | CDH20 | 37 | 18 |
| cg21633052 | 2.92E−09 | 1.24E−03 | −0.204149696 | 0.204149696 | loss | 0.29022603 | 0.086076334 | WWC2-AS2 | 37 | 4 |
| cg21846790 | 1.73E−09 | 7.34E−04 | −0.204143572 | 0.204143572 | loss | 0.336986879 | 0.132843308 | UBALD1 | 37 | 16 |
| cg02206767 | 1.22E−08 | 5.16E−03 | −0.204129495 | 0.204129495 | loss | 0.784184925 | 0.580055419 | DRG2 | 37 | 17 |
| cg06797617 | 7.13E−10 | 3.03E−04 | −0.204109353 | 0.204109353 | loss | 0.598332147 | 0.394222794 | | 37 | 2 |
| cg24128434 | 7.13E−10 | 3.03E−04 | −0.204097217 | 0.204097217 | loss | 0.828463717 | 0.6243665 | DNAH2 | 37 | 17 |
| cg02523270 | 3.02E−09 | 1.28E−03 | −0.204089589 | 0.204089589 | loss | 0.359423617 | 0.155334028 | | 37 | 3 |
| cg01947751 | 8.47E−10 | 3.60E−04 | −0.204089068 | 0.204089068 | loss | 0.710573304 | 0.506484235 | MFI2; MPI2-AS1 | 37 | 3 |
| cg19242688 | 8.47E−10 | 3.60E−04 | −0.204069057 | 0.204069057 | loss | 0.274600442 | 0.070531385 | GNG8 | 37 | 19 |
| cg13550419 | 7.13E−10 | 3.03E−04 | −0.20406706 | 0.20406706 | loss | 0.410642313 | 0.206575253 | | 37 | 17 |
| cg16868095 | 7.13E−10 | 3.03E−04 | −0.204023594 | 0.204023594 | loss | 0.646820158 | 0.442796565 | | 37 | 3 |
| cg03623111 | 7.13E−10 | 3.03E−04 | −0.20402213 | 0.20402213 | loss | 0.840354977 | 0.636332847 | ZDHHC1 | 37 | 16 |
| cg25002788 | 7.13E−10 | 3.03E−04 | −0.204013701 | 0.204013701 | loss | 0.630016877 | 0.426003176 | SPON2; SPON2; SPON2; SPON2 | 37 | 4 |
| cg08971217 | 7.48E−09 | 3.18E−03 | −0.204002948 | 0.204002948 | loss | 0.372211577 | 0.168208629 | | 37 | 16 |
| cg13717817 | 5.39E−09 | 2.29E−03 | −0.203964452 | 0.203964452 | loss | 0.768545875 | 0.564581424 | IL17RD | 37 | 3 |
| cg12629515 | 7.13E−10 | 3.03E−04 | −0.203956894 | 0.203956894 | loss | 0.805116364 | 0.601159471 | HIST1H2BO; HIST1H3J | 37 | 6 |
| cg19695978 | 7.13E−10 | 3.03E−04 | −0.203951641 | 0.203951641 | loss | 0.6245456 | 0.420593959 | | 37 | 15 |
| cg01365639 | 1.01E−09 | 4.27E−04 | −0.203945592 | 0.203945592 | loss | 0.579036198 | 0.375090606 | | 37 | 10 |
| cg18152887 | 1.01E−09 | 4.27E−04 | −0.203941984 | 0.203941984 | loss | 0.527111808 | 0.323169824 | | 37 | 2 |
| cg01292846 | 7.13E−10 | 3.03E−04 | −0.203920729 | 0.203920729 | loss | 0.633041558 | 0.429120829 | | 37 | 17 |
| cg01390950 | 7.13E−10 | 3.03E−04 | −0.203918863 | 0.203918863 | loss | 0.465828104 | 0.261909241 | GRM4 | 37 | 6 |
| cg07262247 | 7.13E−10 | 3.03E−04 | −0.203918079 | 0.203918079 | loss | 0.940596326 | 0.736678247 | PDLIM4; PDLIM4 | 37 | 5 |
| cg16620608 | 7.13E−10 | 3.03E−04 | −0.203907747 | 0.203907747 | loss | 0.731876142 | 0.527968394 | MOGAT3 | 37 | 7 |
| cg26345105 | 1.30E−09 | 5.52E−04 | −0.203889528 | 0.203889528 | loss | 0.404458045 | 0.200568518 | | 37 | 1 |
| cg12275060 | 7.13E−10 | 3.03E−04 | −0.203882799 | 0.203882799 | loss | 0.386243981 | 0.182361182 | BTBD19 | 37 | 1 |
| cg02870820 | 7.13E−10 | 3.03E−04 | −0.203881381 | 0.203881381 | loss | 0.514564228 | 0.310682847 | SSC5D; SSC5D | 37 | 19 |
| cg23146472 | 1.54E−08 | 6.56E−03 | −0.203875439 | 0.203875439 | loss | 0.446751662 | 0.242876224 | TTBK1 | 37 | 6 |
| cg00082140 | 7.13E−10 | 3.03E−04 | −0.203858788 | 0.203858788 | loss | 0.427610817 | 0.223752029 | | 37 | 5 |
| cg25845955 | 7.13E−10 | 3.03E−04 | −0.203850257 | 0.203850257 | loss | 0.301312768 | 0.097462511 | PRRT1 | 37 | 5 |
| cg13574820 | 7.13E−10 | 3.03E−04 | −0.203842919 | 0.203842919 | loss | 0.380876408 | 0.177033488 | | 37 | 7 |
| cg03719040 | 7.13E−10 | 3.03E−04 | −0.203836715 | 0.203836715 | loss | 0.503852409 | 0.300015694 | TMEM128 | 37 | 4 |
| cg12584458 | 1.22E−08 | 5.16E−03 | −0.203833807 | 0.203533807 | loss | 0.433161113 | 0.229327306 | | 37 | 6 |
| cg17265358 | 9.23E−10 | 3.92E−04 | −0.20382 | 0.20382 | loss | 0.671913211 | 0.468093212 | CPLX1 | 37 | 4 |
| cg12946440 | 1.19E−09 | 5.07E−04 | −0.203810347 | 0.203810347 | loss | 0.7183021 | 0.514491753 | ELFN1 | 37 | 7 |
| cg05866854 | 5.01E−08 | 2.13E−02 | −0.203788937 | 0.203788937 | loss | 0.766714808 | 0.562925871 | GORASP2; GORASP2 | 37 | 2 |
| cg03811829 | 7.13E−10 | 3.03E−04 | −0.203772787 | 0.203772787 | loss | 0.83845347 | 0.634680682 | ISG15 | 37 | 1 |
| cg26175729 | 7.13E−10 | 3.03E−04 | −0.203750923 | 0.203750923 | loss | 0.75894637 | 0.555195447 | ABCB8; ABCB8; ABCB8; ABCB8 | 37 | 7 |
| cg06961147 | 9.23E−10 | 3.92E−04 | −0.203735008 | 0.203735008 | loss | 0.485164949 | 0.281429941 | TANC1; TANC1 | 37 | 2 |
| cg03254146 | 1.99E−09 | 8.43E−04 | −0.203713792 | 0.203713792 | loss | 0.710699962 | 0.506986171 | | 37 | 1 |
| cg24158324 | 5.41E−08 | 2.30E−02 | −0.2037036 | 0.2037086 | loss | 0.772610558 | 0.568901959 | | 37 | 3 |
| cg26789888 | 1.99E−09 | 8.43E−04 | −0.203703145 | 0.203703145 | loss | 0.902464751 | 0.698761606 | | 37 | 11 |
| cg04524088 | 1.96E−08 | 8.33E−03 | −0.20368251 | 0.20368251 | loss | 0.607289551 | 0.403607141 | MIR129-1 | 37 | 7 |
| cg01124539 | 7.13E−10 | 3.03E−04 | −0.203673267 | 0.203673267 | loss | 0.729655879 | 0.525982612 | | 37 | 9 |
| cg02876276 | 7.84E−10 | 3.33E−04 | −0.203632741 | 0.203632741 | loss | 0.9871001 | 0.783467359 | | 37 | 17 |
| cg02121529 | 3.02E−09 | 1.28E−03 | −0.203590305 | 0.203590305 | loss | 0.314112572 | 0.110522267 | | 37 | 13 |
| cg13447295 | 9.22E−08 | 3.91E−02 | −0.203569697 | 0.203569697 | loss | 0.888969268 | 0.685399571 | | 37 | 6 |
| cg14259717 | 5.39E−09 | 2.29E−03 | −0.203564452 | 0.203564452 | loss | 0.538996211 | 0.335431759 | LDHC; LDHC | 37 | 11 |
| cg06029792 | 3.57E−09 | 1.52E−03 | −0.203556077 | 0.203556077 | loss | 0.866203694 | 0.662647618 | | 37 | 15 |
| cg02250764 | 7.84E−10 | 3.33E−04 | −0.203552317 | 0.203552317 | loss | 0.518927252 | 0.315374935 | | 37 | 5 |
| cg24091438 | 2.06E−09 | 8.75E−04 | −0.203541634 | 0.203541634 | loss | 0.340535938 | 0.136994304 | LRRC61; ACTR3C; LRRC61 | 37 | 7 |
| cg06193239 | 7.77E−10 | 3.30E−04 | −0.203541151 | 0.203541151 | loss | 0.723967845 | 0.520426694 | | 37 | 11 |
| cg23853507 | 4.97E−09 | 2.11E−03 | −0.203522178 | 0.203522178 | loss | 0.427490213 | 0.223968035 | ADARB2 | 37 | 10 |
| cg11237792 | 8.47E−10 | 3.60E−04 | −0.203516735 | 0.203516735 | loss | 0.5768511 | 0.373344365 | TTC30A | 37 | 2 |
| cg03670393 | 7.13E−10 | 3.03E−04 | −0.20350997 | 0.20350997 | loss | 0.439299775 | 0.235789806 | | 37 | 4 |
| cg13672871 | 1.19E−09 | 5.07E−04 | −0.203495457 | 0.203495457 | loss | 0.487100204 | 0.283604747 | | 37 | 13 |
| cg04855401 | 7.13E−10 | 3.03E−04 | −0.203486013 | 0.203486013 | loss | 0.283211851 | 0.079725838 | | 37 | 2 |
| cg05498866 | 1.54E−09 | 6.54E−04 | −0.203454191 | 0.203454191 | loss | 0.615317732 | 0.411863541 | SFTPC; SFTPC; SFTPC | 37 | 8 |
| cg13390800 | 9.23E−10 | 3.92E−04 | −0.20344244 | 0.20344244 | loss | 0.71643394 | 0.5129915 | DCHS1 | 37 | 11 |
| cg05015938 | 1.42E−09 | 6.01E−04 | −0.203436035 | 0.203436035 | loss | 0.784222764 | 0.580786729 | FNDC1 | 37 | 6 |
| cg14494090 | 7.13E−10 | 3.03E−04 | −0.203419848 | 0.203419848 | loss | 0.395762072 | 0.193342224 | KNDC1 | 37 | 10 |
| cg04648334 | 1.42E−09 | 6.01E−04 | −0.203419138 | 0.203419138 | loss | 0.630672932 | 0.427253794 | | 37 | 6 |
| cg11924019 | 1.68E−09 | 7.12E−04 | −0.203411477 | 0.203411477 | loss | 0.463848083 | 0.260436606 | CYP1A1 | 37 | 15 |
| cg09811860 | 8.47E−10 | 3.60E−04 | −0.203402176 | 0.203402176 | loss | 0.516703558 | 0.313301382 | | 37 | 1 |
| cg01251360 | 1.96E−08 | 8.33E−03 | −0.203393756 | 0.203393756 | loss | 0.608173332 | 0.404779576 | CDH1 | 37 | 16 |
| cg04375683 | 7.13E−10 | 3.03E−04 | −0.203392902 | 0.203392902 | loss | 0.503713202 | 0.3003203 | PPP2R1A; PPP2R1A | 37 | 19 |
| cg07139196 | 1.42E−09 | 6.01E−04 | −0.203383418 | 0.203383418 | loss | 0.79805583 | 0.594672412 | | 37 | 12 |
| cg26883837 | 7.77E−10 | 3.30E−04 | −0.203356033 | 0.203356033 | loss | 0.838443851 | 0.635087818 | | 37 | 21 |
| cg07954075 | 1.19E−09 | 5.07E−04 | −0.203337823 | 0.203337823 | loss | 0.826751817 | 0.623413994 | | 37 | 12 |
| cg22867608 | 1.96E−08 | 8.33E−03 | −0.203333209 | 0.203333209 | loss | 0.922066226 | 0.718733018 | EGR2; EGR2; EGR2; EGR2 | 37 | 10 |
| cg02698886 | 7.13E−10 | 3.03E−04 | −0.203291165 | 0.203291165 | loss | 0.647345936 | 0.444054771 | DCDC2 | 37 | 6 |
| cg18755942 | 7.13E−10 | 3.03E−04 | −0.203273788 | 0.203273788 | loss | 0.832974464 | 0.629700676 | C3orf72; FOXL2 | 37 | 3 |
| cg09648727 | 1.83E−09 | 7.75E−04 | −0.20326697 | 0.20326697 | loss | 0.785417164 | 0.582150194 | | 37 | 17 |
| cg16260421 | 7.13E−10 | 3.03E−04 | −0.203246846 | 0.203246846 | loss | 0.38312387 | 0.179877024 | AGBL4 | 37 | 1 |
| cg07014308 | 1.68E−09 | 7.12E−04 | −0.203242621 | 0.203242621 | loss | 0.889893157 | 0.686650535 | C6orf25; C6orf25; C6orf25; | 37 | 6 |
| | | | | | | | | C6orf25; C6orf25; C6orf25 | | |
| cg18417954 | 1.96E−08 | 8.33E−03 | 0.20323902 | 0.20323902 | gain | 0.240218909 | 0.443457929 | DNAAF3; DNAAF3; DNAAF3; | 37 | 19 |
| | | | | | | | | DNAAF3 | | |
| cg10754705 | 2.35E−09 | 9.98E−04 | −0.20323734 | 0.20323734 | loss | 0.418076542 | 0.214839201 | PHYHIP; PHYHIP | 37 | 8 |
| cg05142520 | 7.13E−10 | 3.03E−04 | −0.203233204 | 0.203233204 | loss | 0.424464909 | 0.221231706 | WNT10B | 37 | 12 |
| cg08044427 | 3.57E−09 | 1.52E−03 | −0.203226531 | 0.203226531 | loss | 0.542601919 | 0.339375388 | | 37 | 2 |
| cg12716837 | 4.55E−09 | 1.93E−03 | −0.203210515 | 0.203210515 | loss | 0.466179746 | 0.262969231 | SORCS2 | 37 | 4 |
| cg14315430 | 7.13E−10 | 3.03E−04 | −0.203201168 | 0.203201168 | loss | 0.613203226 | 0.410002059 | DDIT3; DDIT3; DDIT3; DDIT3; | 37 | 12 |
| | | | | | | | | DDIT3; DDIT3; MBD6 | | |
| cg02798848 | 7.77E−10 | 3.30E−04 | −0.203189247 | 0.203189247 | loss | 0.602344042 | 0.399154794 | | 37 | 7 |
| cg01923075 | 7.13E−10 | 3.03E−04 | −0.203176527 | 0.203176527 | loss | 0.824743309 | 0.621566782 | | 37 | 7 |
| cg06629734 | 7.13E−10 | 3.03E−04 | −0.203173451 | 0.203173451 | loss | 0.463839409 | 0.260665959 | GLP1R | 37 | 6 |
| cg27615366 | 7.13E−10 | 3.03E−04 | −0.203159626 | 0.203159626 | loss | 0.539252591 | 0.336092965 | PDLIM4; PDLIM4 | 37 | 5 |
| cg19211931 | 3.02E−09 | 1.28E−03 | −0.203138221 | 0.203138221 | loss | 0.426114426 | 0.222976206 | DPP10; DPP10 | 37 | 2 |
| cg17793372 | 1.42E−09 | 6.01E−04 | −0.203101535 | 0.203101535 | loss | 0.669298964 | 0.466197429 | SRMS; SRMS | 37 | 20 |
| cg11468148 | 8.47E−10 | 3.60E−04 | −0.20309552 | 0.20309552 | loss | 0.823028432 | 0.619932912 | DIP2C | 37 | 10 |
| cg16777618 | 3.88E−09 | 1.65E−03 | −0.20305606 | 0.20305606 | loss | 0.385425708 | 0.182369647 | | 37 | 11 |
| cg25213362 | 7.13E−10 | 3.03E−04 | −0.20302387 | 0.20302387 | loss | 0.655672111 | 0.452648241 | TMPRSS12 | 37 | 12 |
| cg10366786 | 8.80E−09 | 3.74E−03 | −0.203019266 | 0.203019266 | loss | 0.608982719 | 0.405963453 | SNORA26; DANCR; MIR4449 | 37 | 4 |
| cg05051605 | 9.23E−10 | 3.92E−04 | −0.20301698 | 0.20301698 | loss | 0.790671421 | 0.587654441 | VWA3A | 37 | 16 |
| cg08142858 | 7.13E−10 | 3.03E−04 | −0.20300817 | 0.20300817 | loss | 0.758024158 | 0.555015988 | | 37 | 1 |
| cg04252762 | 8.47E−10 | 3.60E−04 | −0.202998175 | 0.202998175 | loss | 0.83674524 | 0.633747065 | | 37 | 1 |
| cg26334166 | 7.84E−10 | 3.33E−04 | −0.202986438 | 0.202986438 | loss | 0.777213085 | 0.574226647 | COL20A1 | 37 | 20 |
| cg26003909 | 7.77E−10 | 3.30E−04 | −0.20298603 | 0.20298603 | loss | 0.595200842 | 0.392214812 | | 37 | 8 |
| cg01146943 | 7.13E−10 | 3.03E−04 | −0.202962109 | 0.202962109 | loss | 0.861333115 | 0.658371006 | DLEU7; DLEU7-AS1 | 37 | 13 |
| cg19260189 | 7.77E−10 | 3.30E−04 | −0.202960233 | 0.202960233 | loss | 0.804606621 | 0.601646388 | RFX4; RFX4; LOC100287944 | 37 | 12 |
| cg23474811 | 1.54E−08 | 6.56E−03 | −0.202951012 | 0.202951012 | loss | 0.5214242 | 0.318473188 | SDK1; SDK1 | 37 | 7 |
| cg18686665 | 4.29E−08 | 1.82E−02 | −0.202918724 | 0.202918724 | loss | 0.903253194 | 0.700334471 | | 37 | 2 |
| cg24438909 | 7.13E−10 | 3.03E−04 | −0.202911639 | 0.202911639 | loss | 0.390866545 | 0.187954906 | NTRK1; INSRR; INSRR | 37 | 1 |
| cg20322862 | 7.13E−10 | 3.03E−04 | −0.202892126 | 0.202892126 | loss | 0.422972491 | 0.220080365 | TGIF1; TGIF1 | 37 | 18 |
| cg26192556 | 1.99E−09 | 8.43E−04 | −0.202883255 | 0.202883255 | loss | 0.627759085 | 0.424575829 | SLCO6A1; SLCO6A1 | 37 | 5 |
| cg16807687 | 1.42E−09 | 6.01E−04 | −0.202866163 | 0.202866163 | loss | 0.694059387 | 0.491193224 | CDHR1; CDHR1 | 37 | 10 |
| cg07018645 | 7.13E−10 | 3.03E−04 | −0.202864712 | 0.202864712 | loss | 0.811008289 | 0.608143576 | LMAN1L | 37 | 15 |
| cg04580443 | 1.10E−09 | 4.65E−04 | −0.20284818 | 0.20284818 | loss | 0.775353957 | 0.572505776 | | 37 | 11 |
| cg26474549 | 3.02E−09 | 1.28E−03 | −0.202842106 | 0.202842106 | loss | 0.5851799 | 0.382337794 | LINC01107 | 37 | 2 |
| cg23239444 | 7.13E−10 | 3.03E−04 | −0.202834547 | 0.202834547 | loss | 0.517037153 | 0.314202606 | PACSIN1 | 37 | 6 |
| cg05845831 | 6.89E−09 | 2.93E−03 | −0.20281584 | 0.20281584 | loss | 0.56957724 | 0.3567614 | | 37 | 2 |
| cg03076771 | 7.13E−10 | 3.03E−04 | −0.202805163 | 0.202805163 | loss | 0.404106151 | 0.201300988 | C20orf24; TGIF2- | 37 | 20 |
| | | | | | | | | C20orf24; C20orf24; C20orf24; | | |
| | | | | | | | | C20orf24 | | |
| cg03305746 | 1.87E−09 | 7.93E−04 | −0.202795648 | 0.202795648 | loss | 0.930170977 | 0.727375329 | | 37 | 2 |
| cg02159289 | 2.56E−09 | 1.09E−03 | −0.202762984 | 0.202762984 | loss | 0.594196008 | 0.391433024 | BCOR; BCOR | 37 | X |
| cg14495399 | 7.13E−10 | 3.03E−04 | −0.202758555 | 0.202758555 | loss | 0.846650549 | 0.643891994 | | 37 | 9 |
| cg21074766 | 1.68E−09 | 7.12E−04 | −0.202746695 | 0.202746695 | loss | 0.42165617 | 0.218909475 | | 37 | 15 |
| cg02376887 | 1.83E−09 | 7.75E−04 | −0.202726592 | 0.202726592 | loss | 0.900275145 | 0.697548553 | OVCH1-AS1; OVCH1- | 37 | 12 |
| | | | | | | | | AS1; OVCH1-AS1 | | |
| cg05098551 | 9.23E−10 | 3.92E−04 | −0.202722538 | 0.202722538 | loss | 0.602667379 | 0.399944841 | FKBP7; PLEKHA3; FKBP7; | 37 | 2 |
| | | | | | | | | MIR548N | | |
| cg06225242 | 7.13E−10 | 3.03E−04 | −0.202712878 | 0.202712878 | loss | 0.819754055 | 0.617041176 | | 37 | 11 |
| cg03748603 | 7.13E−10 | 3.03E−04 | −0.202676747 | 0.202676747 | loss | 0.951184911 | 0.748508165 | | 37 | 19 |
| cg03015592 | 7.13E−10 | 3.03E−04 | −0.202672698 | 0.202672698 | loss | 0.659350398 | 0.4566777 | OTOG | 37 | 11 |
| cg26390598 | 1.67E−08 | 7.10E−03 | 0.202654941 | 0.202654941 | gain | 0.433079689 | 0.635734629 | B3GALT5; B3GALT5; B3GALT5; | 37 | 21 |
| | | | | | | | | B3GALT5; B3GALT5 | | |
| cg00383763 | 2.48E−08 | 1.05E−02 | −0.202651088 | 0.202651088 | loss | 0.6915024 | 0.488851312 | | 37 | 1 |
| cg23405152 | 3.57E−09 | 1.52E−03 | −0.202642148 | 0.202642148 | loss | 0.641194266 | 0.438552118 | | 37 | 2 |
| cg14562060 | 7.13E−10 | 3.03E−04 | −0.202608774 | 0.202608774 | loss | 0.669872268 | 0.467263494 | TCP11; TCP11; TCP11; TCP11; | 37 | 6 |
| | | | | | | | | TCP11; TCP11; TCP11; TCP11; | | |
| | | | | | | | | TCP11; TCP11; TCP1 | | |
| | | | | | | | | TCP11; TCP11; TCP11 | | |
| cg18754388 | 1.19E−09 | 5.07E−04 | −0.202603735 | 0.202603735 | loss | 0.68353877 | 0.480935035 | COL11A2; COL11A2; COL11A2 | 37 | 6 |
| cg27419075 | 8.47E−10 | 3.60E−04 | −0.202600602 | 0.202600602 | loss | 0.871205655 | 0.668605053 | PDE2A; PDE2A; PDE2A; PDE2A | 37 | 11 |
| cg07845392 | 7.13E−10 | 3.03E−04 | −0.202600432 | 0.202600432 | loss | 0.403630732 | 0.2010303 | SLC25A10; SLC25A10; SLC25A10 | 37 | 17 |
| cg18411150 | 3.57E−09 | 1.52E−03 | 0.202592852 | 0.202592852 | gain | 0.191459619 | 0.394052471 | SHANK1 | 37 | 19 |
| cg07769947 | 2.48E−08 | 1.05E−02 | −0.202591895 | 0.202591895 | loss | 0.542001436 | 0.339409541 | | 37 | 2 |
| cg01687862 | 7.13E−10 | 3.03E−04 | −0.202585705 | 0.202585705 | loss | 0.672520675 | 0.469934971 | | 37 | 7 |
| cg13516209 | 1.30E−09 | 5.52E−04 | −0.202555284 | 0.202555284 | loss | 0.275881569 | 0.074326286 | HLA-H | 37 | 6 |
| cg19002763 | 7.13E−10 | 3.03E−04 | −0.20254488 | 0.20254488 | loss | 0.746480515 | 0.543935635 | DMTN | 37 | 8 |
| cg01948047 | 1.19E−08 | 5.04E−03 | −0.202537327 | 0.202537327 | loss | 0.921598606 | 0.719061279 | | 37 | 22 |
| cg18402261 | 7.13E−10 | 3.03E−04 | −0.202535961 | 0.202535961 | loss | 0.379975791 | 0.177439829 | | 37 | 17 |
| cg21298759 | 1.30E−09 | 5.52E−04 | −0.20253574 | 0.20253574 | loss | 0.428068281 | 0.225532541 | STPG1; STPG1; STPG1; STPG1; | 37 | 1 |
| | | | | | | | | NIPAL3; STPG1 | | |
| cg03006527 | 7.13E−10 | 3.03E−04 | −0.202534979 | 0.202534979 | loss | 0.471718379 | 0.2691834 | | 37 | 1 |
| cg09263059 | 7.13E−10 | 3.03E−04 | −0.202525168 | 0.202525168 | loss | 0.449078226 | 0.246553059 | | 37 | 17 |
| cg04095097 | 7.34E−08 | 3.12E−02 | −0.202519684 | 0.202519684 | loss | 0.36388466 | 0.161364976 | | 37 | 10 |
| cg18031880 | 8.80E−09 | 3.74E−03 | −0.202482804 | 0.202482804 | loss | 0.52821784 | 0.325735035 | | 37 | 11 |
| cg14222229 | 1.03E−08 | 4.39E−03 | 0.202429241 | 0.202429241 | gain | 0.166994642 | 0.369423882 | NBPF23 | 37 | 1 |
| cg01147352 | 7.13E−10 | 3.03E−04 | −0.202416192 | 0.202416192 | loss | 0.30360896 | 0.101192769 | | 37 | 16 |
| cg27309611 | 2.16E−09 | 9.17E−04 | −0.202398523 | 0.202398523 | loss | 0.425777864 | 0.223379341 | SEPT8; SEPT8; SEPT8; SEPT8; | 37 | 5 |
| | | | | | | | | SEPT8 | | |
| cg09900722 | 9.23E−10 | 3.92E−04 | −0.202390195 | 0.202390195 | loss | 0.891993319 | 0.685603124 | | 37 | 10 |
| cg09120934 | 7.48E−09 | 3.18E−03 | −0.202368509 | 0.202368509 | loss | 0.498736309 | 0.2963678 | POU2F3; POU2F3 | 37 | 11 |
| cg00945260 | 7.13E−10 | 3.03E−04 | −0.202352126 | 0.202352126 | loss | 0.774081591 | 0.571729465 | | 37 | 16 |
| cg03893692 | 7.13E−10 | 3.03E−04 | −0.202319904 | 0.202319904 | loss | 0.737443945 | 0.535124041 | CAMK2A; CAMK2A; CAMK2A; | 37 | 5 |
| | | | | | | | | CAMK2A | | |
| cg03070187 | 7.13E−10 | 3.03E−04 | −0.202312772 | 0.202312772 | loss | 0.547705555 | 0.345392782 | TRIP10 | 37 | 19 |
| cg22704845 | 3.28E−09 | 1.39E−03 | −0.20230171 | 0.20230171 | loss | 0.478164204 | 0.275862494 | | 37 | 10 |
| cg25004981 | 3.67E−08 | 1.56E−02 | −0.202299648 | 0.202299648 | loss | 0.828144283 | 0.625544635 | CERS3; CERS3 | 37 | 15 |
| cg27113326 | 3.94E−08 | 1.67E−02 | −0.202286418 | 0.202286418 | loss | 0.88080566 | 0.678519241 | PTPRN2; PTPRN2; PTPRN2 | 37 | 7 |
| cg03669698 | 1.01E−09 | 4.27E−04 | −0.20226293 | 0.20226293 | loss | 0.797491283 | 0.595228353 | GAS2L1; GAS2L1; GAS2L1 | 37 | 22 |
| cg26679004 | 1.01E−09 | 4.27E−04 | −0.202248992 | 0.202248992 | loss | 0.304266087 | 0.102017095 | GRID1 | 37 | 10 |
| cg05399320 | 1.01E−09 | 4.27E−04 | −0.202240325 | 0.202240325 | loss | 0.479598855 | 0.277358529 | SLC6A5 | 37 | 11 |
| cg21917728 | 1.73E−09 | 7.34E−04 | −0.202171189 | 0.202171189 | loss | 0.310129517 | 0.107958328 | | 37 | 6 |
| cg13095450 | 7.13E−10 | 3.03E−04 | −0.202151639 | 0.202151639 | loss | 0.483398257 | 0.281246618 | | 37 | 17 |
| cg11580525 | 7.13E−10 | 3.03E−04 | −0.202132382 | 0.202132382 | loss | 0.76493787 | 0.562805488 | SCN4B; SCN4B | 37 | 11 |
| cg21221545 | 8.47E−10 | 3.60E−04 | −0.202115452 | 0.202115452 | loss | 0.937233664 | 0.735118212 | | 37 | 2 |
| cg27438425 | 7.13E−10 | 3.03E−04 | −0.202113525 | 0.202113525 | loss | 0.476270666 | 0.274157141 | | 37 | 1 |
| cg00843634 | 2.12E−08 | 9.01E−03 | −0.20210145 | 0.20210145 | loss | 0.725877321 | 0.523775871 | SOX7 | 37 | 8 |
| cg14663607 | 7.13E−10 | 3.03E−04 | −0.202092648 | 0.202092648 | loss | 0.790592183 | 0.588499535 | WNT10A | 37 | 2 |
| cg06187336 | 7.13E−10 | 3.03E−04 | −0.202090972 | 0.202090972 | loss | 0.53593666 | 0.333845688 | MAFA | 37 | 8 |
| cg23170347 | 7.13E−10 | 3.03E−04 | −0.202080923 | 0.202080923 | loss | 0.608073364 | 0.405992441 | | 37 | 3 |
| cg01596963 | 7.13E−10 | 3.03E−04 | −0.202055613 | 0.202055613 | loss | 0.53421136 | 0.332155747 | LRRN4CL | 37 | 11 |
| cg10486974 | 9.23E−10 | 3.92E−04 | −0.20202919 | 0.20202919 | loss | 0.789611002 | 0.587581812 | PACSIN1; PACSIN1 | 37 | 6 |
| cg27217253 | 1.42E−09 | 6.01E−04 | −0.202023977 | 0.202023977 | loss | 0.824607847 | 0.622583871 | CCDC42; CCDC42 | 37 | 17 |
| cg04650834 | 3.57E−09 | 1.52E−03 | −0.202023131 | 0.202023131 | loss | 0.670091402 | 0.468068271 | | 37 | 8 |
| cg16900255 | 7.13E−10 | 3.03E−04 | −0.202017035 | 0.202017035 | loss | 0.431209164 | 0.229192129 | C15orf59 | 37 | 15 |
| cg19408398 | 1.83E−09 | 7.75E−04 | −0.201993728 | 0.201993728 | loss | 0.638489998 | 0.436496271 | FIP1L1; FIP1L1; FIP1L1 | 37 | 4 |
| cg23830810 | 7.34E−08 | 3.12E−02 | −0.201992354 | 0.201992354 | loss | 0.296670506 | 0.094678151 | COQS | 37 | 12 |
| cg10554436 | 7.13E−10 | 3.03E−04 | −0.201980823 | 0.201980823 | loss | 0.883433453 | 0.681452629 | C3orf72; FOXL2 | 37 | 3 |
| cg19478079 | 2.48E−08 | 1.05E−02 | −0.201951148 | 0.201951148 | loss | 0.767426489 | 0.565475341 | | 37 | 17 |
| cg01320298 | 7.13E−10 | 3.03E−04 | −0.201949439 | 0.201949439 | loss | 0.563481615 | 0.361532176 | | 37 | 3 |
| cg11405475 | 8.47E−10 | 3.60E−04 | −0.201935358 | 0.201935358 | loss | 0.906573081 | 0.704637724 | SNRPE | 37 | 1 |
| cg12116535 | 1.43E−08 | 6.06E−03 | −0.201900861 | 0.201900861 | loss | 0.565045149 | 0.363144288 | MCF2; MCF2 | 37 | X |
| cg13910377 | 4.21E−09 | 1.79E−03 | −0.201861062 | 0.201861062 | loss | 0.514097257 | 0.312236194 | LINC00111 | 37 | 21 |
| cg22273182 | 7.13E−10 | 3.03E−04 | −0.20185195 | 0.20185195 | loss | 0.479089726 | 0.277237776 | | 37 | 14 |
| cg07160871 | 8.11E−09 | 3.44E−03 | −0.201829218 | 0.201829218 | loss | 0.380878545 | 0.179049328 | | 37 | 2 |
| cg01489256 | 1.81E−08 | 7.69E−03 | −0.201818133 | 0.201818133 | loss | 0.743731315 | 0.541913182 | TDH | 37 | 8 |
| cg23289456 | 1.30E−09 | 5.52E−04 | −0.201816793 | 0.201816793 | loss | 0.693175875 | 0.491359082 | SLC52A3 | 37 | 20 |
| cg13560058 | 7.13E−10 | 3.03E−04 | −0.201807081 | 0.201807081 | loss | 0.821712334 | 0.619905253 | | 37 | 1 |
| cg26580609 | 1.12E−08 | 4.76E−03 | −0.201802847 | 0.201802847 | loss | 0.912943464 | 0.711140618 | NT5C1B; NT5C1B; NT5C1B; | 37 | 2 |
| | | | | | | | | NT5C1B; NT5C1B- | | |
| | | | | | | | | RDH14; NT5C1B- | | |
| | | | | | | | | RDH14; NT5C1B | | |
| cg21995147 | 7.77E−10 | 3.30E−04 | −0.201780822 | 0.201780822 | loss | 0.964887298 | 0.763106476 | ABCA7 | 37 | 19 |
| cg15546638 | 2.30E−08 | 9.75E−03 | −0.201750619 | 0.201750619 | loss | 0.510707472 | 0.308956853 | | 37 | 15 |
| cg16574155 | 8.47E−10 | 3.60E−04 | −0.201740859 | 0.201740859 | loss | 0.793201947 | 0.591461088 | C1orf172 | 37 | 1 |
| cg12499062 | 8.47E−10 | 3.60E−04 | −0.201730787 | 0.201730787 | loss | 0.959990487 | 0.7582597 | | 37 | 18 |
| cg07511014 | 4.97E−09 | 2.11E−03 | −0.201730454 | 0.201730454 | loss | 0.305353968 | 0.103623514 | PAOX; PAOX; PAOX | 37 | 10 |
| cg14688662 | 7.48E−09 | 3.18E−03 | −0.201719392 | 0.201719392 | loss | 0.588871804 | 0.387152412 | | 37 | 7 |
| cg06653203 | 1.54E−09 | 6.54E−04 | −0.201694401 | 0.201694401 | loss | 0.68456103 | 0.482866629 | WNT7A | 37 | 3 |
| cg18331412 | 3.57E−09 | 1.52E−03 | −0.201684655 | 0.201684655 | loss | 0.546988138 | 0.345303482 | TGFBI | 37 | 5 |
| cg11437223 | 8.22E−08 | 3.49E−02 | −0.201673692 | 0.201673692 | loss | 0.369939227 | 0.168265535 | DPY19L1 | 37 | 7 |
| cg16091553 | 1.07E−07 | 4.55E−02 | −0.201659638 | 0.201669638 | loss | 0.365803558 | 0.164133921 | GABRG1; GABRG1 | 37 | 4 |
| cg26228746 | 7.77E−10 | 3.30E−04 | −0.201649821 | 0.201649821 | loss | 0.633594204 | 0.431944382 | LICMA; LICMA | 37 | 10 |
| cg09269848 | 7.13E−10 | 3.03E−04 | −0.201640337 | 0.201640337 | loss | 0.351268119 | 0.149627782 | MYO1C; MYO1C; MYO1C | 37 | 17 |
| cg19360482 | 7.13E−10 | 3.03E−04 | −0.201639697 | 0.201639697 | loss | 0.732932979 | 0.531293282 | | 37 | 5 |
| cg01195404 | 7.84E−10 | 3.33E−04 | −0.201628905 | 0.201628905 | loss | 0.881554658 | 0.679925753 | | 37 | 2 |
| cg01446692 | 7.92E−08 | 3.36E−02 | −0.201595321 | 0.201595321 | loss | 0.785054104 | 0.583458782 | CER1 | 37 | 9 |
| cg11615607 | 2.35E−09 | 9.98E−04 | −0.201592199 | 0.201592199 | loss | 0.907844681 | 0.706252482 | | 37 | 3 |
| cg01120874 | 7.13E−10 | 3.03E−04 | −0.201578773 | 0.201578773 | loss | 0.297622115 | 0.096043342 | FIBCD1; FIBCD1 | 37 | 9 |
| cg06983508 | 7.77E−10 | 3.30E−04 | −0.201532531 | 0.201532531 | loss | 0.557618455 | 0.356085924 | SLC12A8; SLC12A8 | 37 | 3 |
| cg14607660 | 8.47E−10 | 3.60E−04 | −0.201518552 | 0.201518552 | loss | 0.743192211 | 0.541673659 | | 37 | 9 |
| cg24387126 | 7.77E−10 | 3.30E−04 | −0.201489906 | 0.201489906 | loss | 0.542694006 | 0.3412041 | GATA4; C8orf49 | 37 | 8 |
| cg10552903 | 7.13E−10 | 3.03E−04 | −0.201468208 | 0.201468208 | loss | 0.701498649 | 0.500030441 | BCL7A; BCL7A | 37 | 12 |
| cg26973074 | 3.88E−09 | 1.65E−03 | −0.201460545 | 0.201460545 | loss | 0.769678592 | 0.568218047 | KIAA1210 | 37 | X |
| cg24222546 | 1.10E−09 | 4.55E−04 | −0.201452513 | 0.201452613 | loss | 0.59301066 | 0.391558047 | STRA6; STRA6; STRA6; STRA6; | 37 | 15 |
| | | | | | | | | STRA6 | | |
| cg01916632 | 9.23E−10 | 3.92E−04 | −0.201430725 | 0.201430725 | loss | 0.565807666 | 0.364376941 | PDSS2 | 37 | 6 |
| cg11086695 | 6.30E−08 | 2.68E−02 | −0.20142368 | 0.20142368 | loss | 0.624183157 | 0.422759476 | | 37 | 7 |
| cg22973459 | 7.13E−10 | 3.03E−04 | −0.201411238 | 0.201411238 | loss | 0.838996243 | 0.637585006 | | 37 | 10 |
| cg24682986 | 4.97E−09 | 2.11E−03 | −0.2014093 | 0.2014093 | loss | 0.815097747 | 0.613688447 | | 37 | 2 |
| cg13359765 | 4.21E−09 | 1.79E−03 | −0.201404717 | 0.201404717 | loss | 0.507005628 | 0.305600912 | SLC35F3 | 37 | 1 |
| cg18452703 | 7.13E−10 | 3.03E−04 | −0.201393627 | 0.201393627 | loss | 0.692259068 | 0.490865441 | | 37 | 10 |
| cg13032057 | 7.13E−10 | 3.03E−04 | −0.20138807 | 0.20138807 | loss | 0.84760927 | 0.6462212 | | 37 | 6 |
| cg18435000 | 3.57E−09 | 1.52E−03 | −0.201387979 | 0.201387979 | loss | 0.684092279 | 0.4827043 | IQSEC3; IQSEC3; LOC574538 | 37 | 12 |
| cg06632797 | 7.13E−10 | 3.03E−04 | −0.20137469 | 0.20137469 | loss | 0.89671286 | 0.695338171 | | 37 | 11 |
| cg27543040 | 4.97E−09 | 2.11E−03 | −0.201365666 | 0.201365666 | loss | 0.353107949 | 0.151742283 | | 37 | 1 |
| cg04912710 | 7.13E−10 | 3.03E−04 | −0.201364362 | 0.201364362 | loss | 0.649905568 | 0.448541206 | | 37 | 8 |
| cg06795125 | 2.69E−08 | 1.14E−02 | −0.201356064 | 0.201356064 | loss | 0.48990447 | 0.288548406 | SLILT1C2; SLILT1C2 | 37 | 2 |
| cg01134643 | 5.01E−08 | 2.13E−02 | −0.201355498 | 0.201355498 | loss | 0.901432162 | 0.700076665 | LICN3 | 37 | 10 |
| cg08633479 | 6.88E−09 | 2.92E−03 | −0.201354814 | 0.201354814 | loss | 0.67537167 | 0.474016856 | LISP29 | 37 | 19 |
| cg26164234 | 7.13E−10 | 3.03E−04 | −0.20134281 | 0.20134281 | loss | 0.872354857 | 0.671012047 | SLIT1; ARHGAP19-SLIT1 | 37 | 10 |
| cg09637273 | 7.77E−10 | 3.30E−04 | −0.201337101 | 0.201337101 | loss | 0.288018868 | 0.086681766 | CLIEDC1; CLIEDC1 | 37 | 17 |
| cg02007434 | 3.88E−09 | 1.65E−03 | −0.201329497 | 0.201329497 | loss | 0.915276638 | 0.713947141 | TLL2 | 37 | 10 |
| cg12705353 | 8.47E−10 | 3.60E−04 | −0.201287797 | 0.201287797 | loss | 0.623206468 | 0.421918671 | WDR66; WDR66 | 37 | 12 |
| cg20704560 | 7.77E−10 | 3.30E−04 | −0.201282799 | 0.201282799 | loss | 0.411099817 | 0.209817018 | CASZ1 | 37 | 1 |
| cg14016344 | 7.13E−10 | 3.03E−04 | −0.201249434 | 0.201249434 | loss | 0.581926875 | 0.380677441 | CD72 | 37 | 9 |
| cg17186388 | 7.33E−10 | 3.03E−04 | −0.201237526 | 0.201237526 | loss | 0.375975185 | 0.174737659 | WIPF3 | 37 | 7 |
| cg13708645 | 1.12E−08 | 4.76E−03 | −0.201234648 | 0.201234648 | loss | 0.493968077 | 0.292733429 | KDM2B; KDM2B | 37 | 12 |
| cg22979368 | 7.77E−10 | 3.30E−04 | −0.201234127 | 0.201234127 | loss | 0.410420704 | 0.209186576 | IGFBP2 | 37 | 2 |
| cg00115747 | 7.13E−10 | 3.03E−04 | −0.201227736 | 0.201227736 | loss | 0.721664094 | 0.520436359 | MGAT5B; MGAT5B; MGAT5B | 37 | 17 |
| cg09230763 | 1.19E−09 | 5.07E−04 | 0.201226925 | 0.201226925 | loss | 0.783667372 | 0.582440447 | MAP3K6 | 37 | 1 |
| cg01283343 | 7.13E−10 | 3.03E−04 | −0.201226544 | 0.201226544 | loss | 0.526122121 | 0.324895576 | CEACAM5 | 37 | 19 |
| cg06414816 | 3.97E−08 | 1.69E−02 | −0.201218183 | 0.201218183 | loss | 0.494444642 | 0.293226459 | TMEM232 | 37 | 5 |
| cg00253658 | 3.02E−09 | 1.28E−03 | −0.201211546 | 0.201211546 | loss | 0.351219511 | 0.150007965 | | 37 | 16 |
| cg07832006 | 8.47E−10 | 3.60E−04 | −0.201194481 | 0.201194481 | loss | 0.809395828 | 0.608201347 | SYCP1; SYCP1; SYCP1; SYCP1 | 37 | 1 |
| cg09359017 | 7.13E−10 | 3.03E−04 | −0.201155744 | 0.201155744 | loss | 0.855251668 | 0.654095924 | | 37 | 1 |
| cg17840408 | 1.96E−08 | 8.33E−03 | −0.20113906 | 0.20113906 | loss | 0.489877572 | 0.288738512 | PKP3 | 37 | 11 |
| cg16289210 | 1.07E−07 | 4.55E−02 | −0.201090812 | 0.201090812 | loss | 0.395945542 | 0.194854729 | FMOD; FMOD; FMOD; FMOD | 37 | 1 |
| cg04202736 | 2.30E−08 | 9.75E−03 | −0.201083282 | 0.201083282 | loss | 0.900605811 | 0.699522529 | PLA2G4D | 37 | 15 |
| cg26100214 | 6.30E−08 | 2.68E−02 | −0.201079223 | 0.201079223 | loss | 0.754820306 | 0.553741082 | | 37 | 8 |
| cg17211562 | 7.13E−10 | 3.03E−04 | −0.201070698 | 0.201070698 | loss | 0.63096064 | 0.429889941 | LPIN1; LPIN1; LPIN1; LPN1 | 37 | 2 |
| cg25097610 | 3.28E−09 | 1.39E−03 | −0.201024417 | 0.201024417 | loss | 0.658813858 | 0.457789441 | | 37 | 4 |
| cg10043663 | 7.13E−10 | 3.03E−04 | −0.201011877 | 0.201011877 | loss | 0.749274994 | 0.548263118 | ASIC4 | 37 | 2 |
| cg19904265 | 5.01E−08 | 2.13E−02 | −0.201001779 | 0.201001779 | loss | 0.667049079 | 0.4660473 | PRDM16; PRDM16 | 37 | 1 |
| cg24122922 | 3.02E−09 | 1.28E−03 | −0.20099653 | 0.20099653 | loss | 0.63632113 | 0.4353246 | SYNDIG1 | 37 | 20 |
| cg02356435 | 1.01E−09 | 4.27E−04 | −0.200984041 | 0.200984041 | loss | 0.713250958 | 0.512266918 | PLCL2 | 37 | 3 |
| cg00422578 | 2.69E−08 | 1.14E−02 | −0.20095293 | 0.20095293 | loss | 0.612441477 | 0.411488547 | | 37 | 11 |
| cg14021880 | 7.13E−10 | 3.03E−04 | −0.200935111 | 0.200935111 | loss | 0.704732511 | 0.5037974 | EMILIN1 | 37 | 2 |
| cg22856010 | 2.30E−08 | 9.75E−03 | −0.200919814 | 0.200919814 | loss | 0.663028455 | 0.462108641 | | 37 | 8 |
| cg24730688 | 7.13E−10 | 3.03E−04 | −0.200907773 | 0.200907773 | loss | 0.536147432 | 0.335239659 | KIF17; KIF17 | 37 | 1 |
| cg17301379 | 1.03E−08 | 4.39E−03 | −0.200881654 | 0.200881654 | loss | 0.870592072 | 0.669710418 | TTC40 | 37 | 10 |
| cg18782651 | 2.16E−09 | 9.17E−04 | −0.200879808 | 0.200879808 | loss | 0.749179638 | 0.548299829 | | 37 | 7 |
| cg18748499 | 7.13E−10 | 3.03E−04 | −0.200879632 | 0.200879632 | loss | 0.314185206 | 0.113305574 | GJD3 | 37 | 17 |
| cg13504318 | 3.40E−08 | 1.44E−02 | −0.20087943 | 0.20087943 | loss | 0.824971489 | 0.624092059 | INSL3; INSL3 | 37 | 19 |
| cg04802386 | 1.19E−09 | 5.07E−04 | −0.200857212 | 0.200857212 | loss | 0.810172294 | 0.609315082 | SLC9B2; SLC9B2 | 37 | 4 |
| cg02962521 | 7.13E−10 | 3.03E−04 | −0.200819308 | 0.200819308 | loss | 0.582896843 | 0.382077535 | | 37 | 18 |
| cg07111415 | 7.77E−10 | 3.30E−04 | −0.200799081 | 0.200799081 | loss | 0.697311334 | 0.496512253 | ZDHHC19; ZDHHC19 | 37 | 3 |
| cg23800435 | 1.10E−09 | 4.65E−04 | −0.200787002 | 0.200787002 | loss | 0.846615996 | 0.645828994 | C1orf210; C1orf210; C1orf210; | 37 | 1 |
| | | | | | | | | C1orf210 | | |
| cg01643605 | 7.13E−10 | 3.03E−04 | −0.200743811 | 0.200743811 | loss | 0.405641664 | 0.204897853 | | 37 | 16 |
| cg07464674 | 7.13E−10 | 3.03E−04 | −0.200729559 | 0.200729559 | loss | 0.426721606 | 0.225992047 | PITPNM3; PITPNM3 | 37 | 17 |
| cg18530107 | 8.47E−10 | 3.60E−04 | −0.200707519 | 0.200707519 | loss | 0.850731825 | 0.650024306 | LOC100188947 | 37 | 10 |
| cg23586996 | 7.13E−10 | 3.03E−04 | −0.200689016 | 0.200689016 | loss | 0.918833287 | 0.718144271 | | 37 | 17 |
| cg02989257 | 7.13E−10 | 3.03E−04 | −0.20068112 | 0.20068112 | loss | 0.776059579 | 0.575378459 | COL16A1 | 37 | 1 |
| cg12923268 | 1.67E−08 | 7.10E−03 | −0.200658035 | 0.200658035 | loss | 0.785815564 | 0.585157529 | C6orf25; C6orf25; C6orf25; | 37 | 6 |
| | | | | | | | | C6orf25; C6orf25;C6orf25 | | |
| cg04993130 | 7.13E−10 | 3.03E−04 | −0.200656851 | 0.200656851 | loss | 0.471047645 | 0.270390794 | EPS8L2 | 37 | 11 |
| cg23708717 | 7.13E−10 | 3.03E−04 | −0.200630985 | 0.200630985 | loss | 0.388921532 | 0.188290547 | CCDC11 | 37 | 18 |
| cg19164987 | 8.47E−10 | 3.60E−04 | −0.200627582 | 0.200627582 | loss | 0.311835985 | 0.111208403 | HOXC4; HOXC6; HOXC5 | 37 | 12 |
| cg14910288 | 7.13E−10 | 3.03E−04 | −0.200626751 | 0.200626751 | loss | 0.551669692 | 0.351042941 | | 37 | 18 |
| cg13799544 | 7.77E−10 | 3.30E−04 | −0.20061813 | 0.20061813 | loss | 0.42609253 | 0.2254744 | FAM189A1 | 37 | 15 |
| cg13993218 | 7.13E−10 | 3.03E−04 | −0.200593235 | 0.200593235 | loss | 0.730729047 | 0.530135812 | INS; INS-IGF2; INS; INS; INS- | 37 | 11 |
| | | | | | | | | IGF2 | | |
| cg18901140 | 7.13E−10 | 3.03E−04 | −0.200586121 | 0.200586121 | loss | 0.523450309 | 0.322864188 | | 37 | 17 |
| cg03719509 | 7.13E−10 | 3.03E−04 | −0.20057271 | 0.20057271 | loss | 0.26887007 | 0.06829736 | LHFPL2 | 37 | 5 |
| cg13218688 | 8.80E−09 | 3.74E−03 | −0.200569269 | 0.200569269 | loss | 0.558371698 | 0.357802429 | FAM69C | 37 | 18 |
| cg22929099 | 5.61E−09 | 2.38E−03 | −0.200553013 | 0.200553013 | loss | 0.91430246 | 0.713749447 | GFRA3 | 37 | 5 |
| cg11004890 | 2.16E−09 | 9.17E−04 | −0.200531989 | 0.200531989 | loss | 0.554735842 | 0.354203853 | SLC4A11; SLC4A11; SLC4A11; | 37 | 20 |
| | | | | | | | | SLC4A11 | | |
| cg09593454 | 7.84E−10 | 3.33E−04 | −0.20052549 | 0.20052549 | loss | 0.886162702 | 0.685637212 | TRPV3; TRPV3 | 37 | 17 |
| cg15841205 | 9.23E−10 | 3.92E−04 | −0.200525324 | 0.200525324 | loss | 0.565049683 | 0.364524359 | | 37 | 14 |
| cg23596567 | 7.13E−10 | 3.03E−04 | −0.200511238 | 0.200511238 | loss | 0.668260838 | 0.4677496 | ZNF18 | 37 | 17 |
| cg25316749 | 8.47E−10 | 3.60E−04 | −0.200488702 | 0.200488702 | loss | 0.474840949 | 0.274352247 | COLCA2; COLCA2; COLCA2; | 37 | 11 |
| | | | | | | | | COLCA1 | | |
| cg18452973 | 3.57E−09 | 1.52E−03 | −0.200483896 | 0.200483896 | loss | 0.457047255 | 0.256563359 | | 37 | 12 |
| cg21535580 | 7.13E−10 | 3.03E−04 | −0.200479873 | 0.200479873 | loss | 0.791275391 | 0.590795518 | ETNK2 | 37 | 1 |
| cg27384088 | 7.13E−10 | 3.03E−04 | −0.200471896 | 0.200471896 | loss | 0.537204608 | 0.336732712 | | 37 | 6 |
| cg07752409 | 1.83E−09 | 7.75E−04 | −0.200463485 | 0.200463485 | loss | 0.800441226 | 0.599977741 | RASGEF1C | 37 | 5 |
| cg02991085 | 1.96E−08 | 8.33E−03 | −0.200449005 | 0.200449005 | loss | 0.268665983 | 0.068216978 | LINC00028 | 37 | 20 |
| cg18758664 | 7.13E−10 | 3.03E−04 | −0.200437219 | 0.200437219 | loss | 0.648934225 | 0.448497006 | MESP2 | 37 | 15 |
| cg14968482 | 7.77E−10 | 3.30E−04 | −0.200424701 | 0.200424701 | loss | 0.919932372 | 0.719507671 | ALOX5; ALOX5; ALOX5 | 37 | 10 |
| cg22896075 | 8.14E−09 | 3.46E−03 | −0.20041798 | 0.20041798 | loss | 0.964674192 | 0.764256213 | GJD3 | 37 | 17 |
| cg13215995 | 7.13E−10 | 3.03E−04 | −0.200415353 | 0.200415353 | loss | 0.635165836 | 0.434750482 | | 37 | 17 |
| cg12285565 | 7.13E−10 | 3.03E−04 | −0.200413791 | 0.200413791 | loss | 0.578680779 | 0.378266988 | | 37 | 8 |
| cg02439077 | 1.01E−09 | 4.27E−04 | −0.200375959 | 0.200375959 | loss | 0.898723183 | 0.698347224 | NODAL | 37 | 10 |
| cg08141989 | 7.77E−10 | 3.30E−04 | −0.200334858 | 0.200334858 | loss | 0.852753517 | 0.652418659 | ASGR1; ASGR1 | 37 | 17 |
| cg21827317 | 6.80E−08 | 2.89E−02 | −0.200322594 | 0.200322594 | loss | 0.3876373 | 0.187314706 | | 37 | 3 |
| cg10333023 | 6.89E−09 | 2.93E−03 | −0.200316914 | 0.200316914 | loss | 0.807046002 | 0.606729088 | KCNJ5; C11orf45; C11orf45; | 37 | 11 |
| | | | | | | | | C11orf45 | | |
| cg26615126 | 7.77E−10 | 3.30E−04 | −0.20030581 | 0.20030581 | loss | 0.753555751 | 0.553249941 | COLEC11; COLEC11; COLEC11; | 37 | 2 |
| | | | | | | | | COLEC11; COLEC11; COLEC11 | | |
| cg02924487 | 7.13E−10 | 3.03E−04 | −0.200287013 | 0.200287013 | loss | 0.68391613 | 0.483629118 | TP73; TP73; TP73; TP73; TP73; | 37 | 1 |
| | | | | | | | | TP73; TP73; TP73; TP73; | | |
| | | | | | | | | TP73; TP73; TP73; TP73 | | |
| cg03848890 | 1.01E−09 | 4.27E−04 | −0.20028403 | 0.20028403 | loss | 0.827988442 | 0.627704412 | OTOF | 37 | 2 |
| cg24059075 | 7.13E−10 | 3.03E−04 | −0.200276511 | 0.200276511 | loss | 0.344473681 | 0.144197171 | PRPH | 37 | 12 |
| cg13451000 | 9.23E−10 | 3.92E−04 | −0.200276317 | 0.200276317 | loss | 0.629003958 | 0.428727641 | LINC00301 | 37 | 11 |
| cg08533674 | 8.47E−10 | 3.60E−04 | −0.200244095 | 0.200244095 | loss | 0.800575136 | 0.600331041 | | 37 | 1 |
| cg26312807 | 5.84E−08 | 2.48E−02 | −0.200244088 | 0.200244088 | loss | 0.404811632 | 0.204567544 | | 37 | 3 |
| cg00851377 | 5.84E−08 | 2.48E−02 | −0.200240391 | 0.200240391 | loss | 0.321279621 | 0.121039229 | GPR85; GPR85; GPR85; GPR85 | 37 | 7 |
| cg17743381 | 7.13E−10 | 3.03E−04 | −0.200237937 | 0.200237937 | loss | 0.389080508 | 0.188842571 | | 37 | 1 |
| cg11830761 | 3.02E−09 | 1.28E−03 | −0.200218819 | 0.200218819 | loss | 0.379187119 | 0.1789683 | | 37 | 3 |
| cg11490745 | 7.48E−09 | 3.18E−03 | −0.200207867 | 0.200207867 | loss | 0.574456185 | 0.474248318 | PAX3; PAX3; PAX3; PAX3;PAX3; | 37 | 2 |
| | | | | | | | | PAX3 | | |
| cg03711791 | 3.12E−09 | 1.32E−03 | −0.200187845 | 0.200187845 | loss | 0.732154104 | 0.531966259 | KCNG1 | 37 | 20 |
| cg20544552 | 4.97E−09 | 2.11E−03 | −0.200185653 | 0.200185653 | loss | 0.293079123 | 0.092893469 | | 37 | 11 |
| cg00451635 | 7.13E−10 | 3.03E−04 | −0.200165315 | 0.200165315 | loss | 0.617881015 | 0.4177157 | EMP2 | 37 | 16 |
| cg01925738 | 2.35E−09 | 9.98E−04 | 0.200152291 | 0.200152291 | gain | 0.515248421 | 0.715400712 | PCDHB3 | 37 | 5 |
| cg26038649 | 6.63E−09 | 2.82E−03 | −0.200150052 | 0.200150052 | loss | 0.717761146 | 0.517611094 | MAP3K6 | 37 | 1 |
| cg00758881 | 7.77E−10 | 3.30E−04 | −0.2001489 | 0.2001489 | loss | 0.219649926 | 0.019501026 | NDRG4; NDRG4; NDRG4; NDRG4; | 37 | 16 |
| | | | | | | | | NDRG4; NDRG4; NDRG4; | | |
| | | | | | | | | NDRG4 | | |
| cg11813441 | 7.13E−10 | 3.03E−04 | −0.200129839 | 0.200129839 | loss | 0.655056709 | 0.454926871 | | 37 | 5 |
| cg06246950 | 7.77E−10 | 3.30E−04 | −0.200089132 | 0.200089132 | loss | 0.466624096 | 0.266534965 | SLC35E4 | 37 | 22 |
| cg04828580 | 2.48E−08 | 1.05E−02 | −0.200084611 | 0.200084611 | loss | 0.775676664 | 0.575592053 | | 37 | 10 |
| cg17338254 | 7.13E−10 | 3.03E−04 | −0.200082328 | 0.200082328 | loss | 0.542521028 | 0.3424387 | TBX1; TBX1; TBX1 | 37 | 22 |
| cg14632490 | 7.48E−09 | 3.18E−03 | −0.200076031 | 0.200076031 | loss | 0.793889819 | 0.593813788 | | 37 | 1 |
| cg22841667 | 1.96E−08 | 8.33E−03 | 0.200061153 | 0.200061153 | gain | 0.407086947 | 0.6071481 | RPL27A; SNORA3; SNORA45 | 37 | 11 |
| cg01922437 | 1.43E−08 | 6.06E−03 | −0.200053632 | 0.200053632 | loss | 0.411954632 | 0.211901 | | 37 | 12 |
| cg10239319 | 4.57E−09 | 1.94E−03 | 0.200053546 | 0.200053546 | gain | 0.45013753 | 0.650191076 | JAKMIP1; JAKMIP1 | 37 | 4 |
| cg23819145 | 7.77E−10 | 3.30E−04 | −0.200028259 | 0.200028259 | loss | 0.648357536 | 0.448329276 | ITSN2; ITSN2; ITSN2 | 37 | 2 |
| cg10938556 | 1.42E−09 | 6.01E−04 | −0.200021717 | 0.200021717 | loss | 0.287416111 | 0.087394394 | | 37 | 14 |

| | Genomic Location | | | Relation to transcription start site |
|---|---|---|---|---|
| Illumina ID | (NCBI, hg19) | Strand | Relation_to_UCSC_CpG_Island | (TSS) |
| cg21105875 | 2.03E+08 | − | N_Shore | KDM5B(tss1500); PCAT6(tss1500) |
| cg23218354 | 2885244 | − | Island | |
| cg07600533 | 50986031 | + | Island | KLHDC7B(tss1500) |
| cg03133378 | 99195931 | − | Island | LOC100289187(body) |
| cg09056876 | 1.57E+08 | + | Island | |
| cg25807487 | 68782106 | − | S_Shelf | MRGPRF(tss1500) |
| cg20079298 | 40572326 | + | Island | |
| cg09684846 | 10912718 | − | Island | TVP23A(tss200) |
| cg12363903 | 1.35E+08 | + | N_Shore | C5orf66(body) |
| cg02956248 | 32120901 | − | N_Shore | LOC100507547(body); PPT2(tss1500); |
| | | | | PPT2- |
| | | | | EGFL8(tss1500); PRRT1(tss1500) |
| cg01996116 | 42846907 | − | S_Shore | HIGD1A(tss1500) |
| cg24365896 | 69974745 | − | | ANO1(body) |
| cg13568106 | 26835115 | + | | |
| cg26863172 | 1361729 | + | Island | TMEM88B(body) |
| cg04015962 | 10949192 | + | | |
| cg00701051 | 1.45E+08 | − | Island | LOC100288142(body); NBPF10(body) |
| cg03930209 | 1.57E+08 | − | Island | |
| cg06704455 | 37815264 | + | N_Shore | ELFN2(body) |
| cg25334934 | 1.21E+08 | − | | |
| cg23689428 | 1.21E+08 | − | | LINC01101(tss200) |
| cg23359665 | 32120907 | − | N_Shore | LOC100507547(body); PPT2(tss1500); |
| | | | | PPT2- |
| | | | | EGFL8(tss1500); PRRT1(tss1500) |
| cg08199758 | 3062426 | − | N_Shore | CLDN9(tss200) |
| cg23731272 | 67356838 | − | N_Shore | SMAD3(tss1500) |
| cg20021506 | 1.57E+08 | + | Island | |
| cg16293892 | 77142620 | − | S_Shore | |
| cg15371801 | 2.32E+08 | + | Island | |
| cg25844921 | 15107309 | − | S_Shore | MRPS25(tss1500) |
| cg26865747 | 28601377 | − | N_Shore | |
| cg07475394 | 79466438 | − | S_Shore | |
| cg16005559 | 1.2E+08 | − | S_Shore | |
| cg11429111 | 1.35E+08 | + | | |
| cg00931644 | 77461368 | + | S_Shore | KCTD12(tss1500) |
| cg12846837 | 48172889 | + | Island | SPIDR(tss1500) |
| cg10316525 | 40572137 | + | Island | |
| cg05820861 | 24882296 | − | Island | C1QTNF9(tss1500) |
| cg08234689 | 1.28E+08 | + | Island | |
| cg16717549 | 45705699 | − | Island | AIRE(tss200) |
| cg00982521 | 41374806 | + | Island | |
| cg06833564 | 4665307 | − | Island | UBALD1(tss1500) |
| cg07816556 | 26017280 | − | N_Shelf | HIST1H1A(body) |
| cg25449950 | 24797174 | + | N_Shelf | SOD3(body) |
| cg18689454 | 45705694 | − | Island | AIRE(tss200) |
| cg09660878 | 77142695 | − | S_Shore | |
| cg12109823 | 67454595 | + | Island | |
| cg11291003 | 1.35E+08 | + | N_Shore | C5orf66(body) |
| cg18315060 | 41374532 | + | Island | |
| cg26942432 | 99557464 | + | N_Shore | |
| cg00186462 | 1E+08 | + | | GPR128(body) |
| cg20422417 | 25427108 | − | Island | |
| cg06729532 | 14376770 | − | Island | |
| cg22344745 | 2.28E+08 | + | Island | |
| cg03274456 | 62273116 | + | | AHNAK(body) |
| cg21795221 | 1.57E+08 | + | Island | |
| cg02486855 | 67356942 | + | N_Shore | SMAD3(tss1500) |
| cg26465389 | 1.35E+08 | + | | |
| cg13504434 | 1.21E+08 | − | | CABP1(body) |
| cg08482837 | 86038423 | − | Island | |
| cg01761236 | 1.03E+08 | − | | |
| cg25968569 | 77461335 | + | S_Shore | KCTD12(tss1500) |
| cg14220678 | 46089024 | + | Island | CCDC17(body) |
| cg19092981 | 19751654 | + | Island | TBX1(body) |
| cg16615454 | 50475167 | + | Island | ASIC1(body) |
| cg08620329 | 67454546 | + | Island | |
| cg02781088 | 1.13E+08 | − | S_Shore | RGPD5(tss1500); RGPD8(tss1500) |
| cg12290671 | 99195819 | − | Island | LOC100289187(tss200) |
| cg07292773 | 1.57E+08 | + | Island | |
| cg05225634 | 1.57E+08 | − | Island | |
| cg25646708 | 1.34E+08 | − | Island | |
| cg13502125 | 1.48E+08 | + | Island | NBPF8(body) |
| cg10690919 | 48912545 | − | | WFIKKN2(tss200) |
| cg15477144 | 45705034 | − | N_Shore | FAM118A(tss1500); FAM118A(tss200) |
| cg12403142 | 92012408 | − | Island | |
| cg02649698 | 43818854 | + | S_Shore | |
| cg11647108 | 81967516 | + | Island | LINC00857(body) |
| cg26915952 | 1.78E+08 | + | Island | |
| cg06686742 | 8273505 | + | N_Shore | CERS4(tss1500) |
| cg03962527 | 1.12E+08 | − | N_Shore | ADD3(body); ADD3(tss1500); ADD3- |
| | | | | AS1(body) |
| cg22249529 | 50985797 | + | Island | KLHDC7B(tss1500) |
| cg26153054 | 1.32E+08 | − | Island | |
| cg00594854 | 48052682 | + | S_Shelf | |
| cg19817652 | 36997420 | + | N_Shore | C17orf98(body) |
| cg18373855 | 1.59E+08 | + | N_Shore | |
| cg04044120 | 73668757 | + | | DNAJB13(body) |
| cg26643967 | 36485282 | + | Island | SDHAF1(tss1500) |
| cg25317631 | 44541905 | + | Island | |
| cg10999992 | 38200221 | + | N_Shore | H1F0(tss1500) |
| cg26576890 | 41374939 | − | Island | |
| cg26070540 | 30225666 | − | Island | COX4I2(tss200) |
| cg12131208 | 58499700 | + | S_Shore | C17orf64(tss200) |
| cg18424208 | 1.5E+08 | − | Island | CDX1(tss200) |
| cg21778193 | 1098070 | − | Island | |
| cg23381884 | 1597525 | + | N_Shore | |
| cg08043592 | 56687388 | − | | GALP(tss200) |
| cg02153814 | 1.94E+08 | + | Island | |
| cg27535677 | 28601365 | − | N_Shore | |
| cg04358214 | 67143304 | + | Island | C16orf70(tss1500) |
| cg07187855 | 30854161 | + | S_Shore | DDR1(body) |
| cg10502244 | 48399295 | − | Island | COL2A1(tss1500) |
| cg21915678 | 59494040 | − | Island | |
| cg26004771 | 1.78E+08 | − | Island | |
| cg09614479 | 81967536 | + | S_Shore | LINC00857(body) |
| cg16073143 | 1.59E+08 | + | N_Shore | |
| cg04157161 | 7906847 | + | Island | GUCY2D(body) |
| cg06154311 | 61002657 | − | | RBBP8NL(tss200) |
| cg12233487 | 2517085 | − | | TMC2(tss200) |
| cg13471599 | 1.21E+08 | + | Island | |
| cg02404974 | 41376295 | + | N_Shore | |
| cg08083251 | 23309930 | − | | |
| cg04561804 | 1.7E+08 | + | N_Shore | LOC100128164(body); SEC62(tss1500) |
| cg14167415 | 77146999 | − | Island | |
| cg04359418 | 76573396 | − | Island | |
| cg26600753 | 1.81E+08 | + | Island | TRIM7(body) |
| cg05412788 | 1.21E+08 | − | Island | |
| cg09593860 | 1.14E+08 | + | N_Shore | ZBTB16(tss1500) |
| cg13614409 | 31506752 | + | | TEX26(tss200); TEX26-AS1(tss200) |
| cg21811021 | 6659345 | − | Island | |
| cg21855361 | 1.28E+08 | + | Island | |
| cg17801295 | 2.42E+08 | + | Island | |
| cg00990385 | 1.14E+08 | − | N_Shelf | CCDC42B(body) |
| cg09044186 | 1.17E+08 | − | Island | APOA5(body) |
| cg22198853 | 1594411 | + | | |
| cg25885280 | 70760166 | − | | SHANK2(body) |
| cg17604655 | 1.57E+08 | + | Island | |
| cg17900689 | 4649262 | + | | ZMYND15(body) |
| cg21010202 | 1615843 | + | Island | |
| cg05168012 | 1.03E+08 | − | Island | |
| cg20137746 | 27235598 | + | | |
| cg17116120 | 30225681 | + | Island | COX4I2(tss200) |
| cg25438517 | 77146796 | + | N_Shore | |
| cg09674170 | 3062382 | − | N_Shore | CLDN9(tss200) |
| cg00970752 | 15711443 | − | | FHAD1(body) |
| cg11117637 | 1.5E+08 | − | Island | CDX1(body) |
| cg06842409 | 30437560 | − | Island | |
| cg05516295 | 1.35E+08 | − | N_Shore | C5orf66(body) |
| cg10365562 | 46615549 | − | N_Shelf | TDGF1(tss1500) |
| cg14706297 | 43328266 | − | Island | |
| cg10676327 | 6659735 | − | Island | |
| cg19196401 | 1.11E+08 | − | Island | DDO(body) |
| cg02632490 | 88024529 | + | S_Shore | GRID1(body); MIR346(body) |
| cg26707664 | 60278353 | + | | |
| cg06423211 | 551401 | − | Island | |
| cg25257677 | 22223985 | − | N_Shore | SLC39A14(tss1500) |
| cg19094530 | 79470169 | − | N_Shore | |
| cg12642717 | 12255318 | − | S_Shore | CIDEA(body) |
| cg20737388 | 73668626 | + | | DNAJB13(body) |
| cg04220541 | 48172992 | − | Island | SPIDR(tss1500) |
| cg26695758 | 32063607 | + | Island | TNXB(body) |
| cg06415891 | 2467096 | − | Island | |
| cg13939055 | 2.44E+08 | − | Island | LOC339529(body) |
| cg20816361 | 2467100 | − | Island | |
| cg00443981 | 58499679 | + | S_Shore | C17orf64(tss200) |
| cg27500148 | 37011491 | − | S_Shore | RPL23(tss1500) |
| cg17976205 | 61002857 | − | | RBBP8NL(tss1500) |
| cg09634707 | 72219713 | + | S_Shore | |
| cg11372436 | 24797007 | − | | SOD3(tss200) |
| cg07537443 | 47572132 | − | N_Shore | NGFR(tss1500) |
| cg00549574 | 74350942 | + | S_Shore | PRPSAP1(tss1500) |
| cg22300566 | 1598558 | − | N_Shore | |
| cg05308495 | 54515169 | − | Island | CACNG6(body) |
| cg06693983 | 55889216 | − | Island | TMEM190(body) |
| cg08379987 | 31507139 | + | | TEX26(body); TEX26-AS1(tss1500) |
| cg09692695 | 74668286 | + | Island | RTKN(body); RTKN(tss1500) |
| cg04202002 | 1.28E+08 | − | N_Shore | |
| cg10635895 | 2339439 | − | Island | CACNA1C(body) |
| cg07405182 | 40598455 | + | N_Shore | |
| cg14429919 | 3054820 | − | | |
| cg00091064 | 41376604 | + | Island | |
| cg10555383 | 1.44E+08 | − | S_Shore | LYNX1(tss1500) |
| cg24574963 | 1.49E+08 | − | S_Shore | |
| cg18568930 | 51815658 | − | S_Shore | IGLON5(body) |
| cg17724412 | 28829283 | − | N_Shelf | LOC401242(body) |
| cg14652587 | 75692571 | − | | |
| cg21406402 | 1.21E+08 | − | | LINC01101(body) |
| cg23924370 | 88583805 | + | Island | |
| cg00643333 | 45705037 | + | N_Shore | FAM118A(tss1500); FAM118A(tss200) |
| cg17034331 | 77146318 | + | N_Shore | |
| cg01952185 | 1.35E+08 | − | | |
| cg00823843 | 2.44E+08 | + | Island | LOC339529(body) |
| cg00944421 | 68269483 | + | Island | ESRP2(body) |
| cg24550212 | 45170494 | − | S_Shore | OCSTAMP(body) |
| cg19940077 | 2.01E+08 | + | Island | GPR25(body) |
| cg18409649 | 99066398 | − | N_Shelf | |
| cg03687532 | 54228358 | − | Island | |
| cg18121224 | 1.77E+08 | − | Island | NSD1(tss1500) |
| cg27102629 | 76573155 | − | N_Shore | |
| cg01342115 | 1.34E+08 | − | Island | |
| cg11559198 | 2.32E+08 | − | Island | |
| cg07581811 | 1.84E+08 | − | | |
| cg00270878 | 64334217 | − | N_Shore | SLC22A11(body) |
| cg14825735 | 1594066 | − | | |
| cg21411366 | 24797176 | + | N_Shelf | SOD3(body) |
| cg20703261 | 41375153 | − | Island | |
| cg19402939 | 1.84E+08 | + | Island | CHRD(body) |
| cg00524708 | 31159558 | + | N_Shore | PRSS36(body) |
| cg02123734 | 50649666 | − | | |
| cg27362048 | 1.17E+08 | − | Island | APOA5(body) |
| cg13713218 | 1E+08 | − | N_shore | LOC100499484(tss1500); LOC100499484- |
| | | | | C9ORF174(tss1500) |
| cg23634477 | 27018112 | − | | |
| cg25066665 | 1.5E+08 | − | N_Shore | RPRD2(tss1500) |
| cg14708990 | 76839217 | + | | MYO7A(tss200) |
| cg06582782 | 5621261 | − | | |
| cg09089451 | 1.49E+08 | − | Island | KRBA1(tss1500) |
| cg11450541 | 24519920 | + | | ANKRD20A19P(body) |
| cg23505823 | 1.23E+08 | + | | HCAR1(tss1500) |
| cg19772114 | 28829321 | − | N_Shelf | LOC401242(body) |
| cg11012412 | 77147141 | − | Island | |
| cg02254407 | 73357159 | − | | PLEKHB1(tss1500); PLEKHB1(tss200) |
| cg17453840 | 83317526 | − | Island | CPEB1(tss1500); LOC283692(body) |
| cg08772588 | 56000201 | + | N_Shore | SSC5D(body) |
| cg04539705 | 2.33E+08 | + | N_Shore | PRSS56(tss1500) |
| cg04942251 | 63687247 | + | S_Shore | |
| cg09288885 | 86066714 | + | | |
| cg10365886 | 32063874 | + | Island | TNXB(body) |
| cg24524285 | 64405919 | − | Island | NRXN2(body) |
| cg21543589 | 30225685 | + | Island | COX4I2(tss200) |
| cg15674899 | 1.51E+08 | + | | |
| cg07242540 | 12600348 | + | Island | |
| cg20084219 | 36997449 | − | Island | C17orf98(body) |
| cg02757970 | 25427350 | + | Island | |
| cg12133451 | 2.28E+08 | + | Island | |
| cg01856384 | 41376904 | − | Island | |
| cg26209990 | 1.28E+08 | − | Island | |
| cg11923631 | 45705721 | − | Island | AIRE(body) |
| cg06303238 | 50418959 | − | Island | SALL4(body) |
| cg03063309 | 24796919 | − | | SOD3(tss200) |
| cg05133205 | 32121249 | − | N_Shore | LOC100507547(body); PPT2(body); |
| | | | | PPT2(tss1500); PPT2- |
| | | | | EGFL8(tss1500) |
| cg05630111 | 1.51E+08 | − | S_Shore | CERS2(tss1500) |
| cg20625967 | 42328655 | − | | PLA2G4E(body) |
| cg10854819 | 31515852 | − | | LOC283710(body) |
| cg27251412 | 45705740 | − | Island | AIRE(body) |
| cg23280506 | 14201938 | + | Island | |
| cg17978631 | 1.84E+08 | + | | |
| cg17261676 | 26016808 | + | N_Shelf | |
| cg20701556 | 95698924 | + | N_Shore | RWDD3(tss1500); TMEM56- |
| | | | | RWDD3(body) |
| cg21235532 | 19476870 | − | Island | |
| cg04159546 | 3605087 | + | N_Shelf | TP73(body) |
| cg26010218 | 78977223 | − | N_Shore | |
| cg01235820 | 642782 | − | Island | SCRT2(body) |
| cg20462561 | 2.29E+08 | − | Island | |
| cg06689045 | 1.2E+08 | − | Island | |
| cg19049754 | 81966990 | − | Island | LINC00857(tss1500) |
| cg06825512 | 10453969 | + | N_Shore | APCDD1(tss1500) |
| cg11985480 | 74729100 | + | N_Shore | LBX2(body); LBX2-AS1(tss1500) |
| cg25457886 | 14339565 | − | Island | |
| cg01205267 | 47126300 | − | N_Shore | LRCH1(tss1500) |
| cg03541791 | 28829396 | − | N_Shelf | LOC401242(body) |
| cg24024505 | 28829401 | − | N_Shelf | LOC401242(body) |
| cg17918558 | 46945595 | − | | |
| cg00003625 | 48912543 | − | | WFIKKN2(tss200) |
| cg01101782 | 2.03E+08 | − | N_Shore | KDM58(tss1500); PCAT6(tss1500) |
| cg23076537 | 93643678 | + | N_shelf | |
| cg20631820 | 95086158 | + | | |
| cg09510752 | 1.35E+08 | + | N_Shore | C5orf66(body) |
| cg02497785 | 48494608 | − | Island | ABCA13(body) |
| cg08247921 | 1.04E+08 | − | S_Shore | MANBA(tss1500) |
| cg04638150 | 62273418 | + | | AHNAK(body) |
| cg08378788 | 1395864 | + | S_Shore | MYO1C(body); MYO1C(tss1500) |
| cg22497095 | 28601375 | − | N_Shore | |
| cg13391079 | 25212556 | + | Island | |
| cg27576576 | 1.57E+08 | − | | |
| cg12799314 | 1405193 | + | Island | ADARB2(body) |
| cg11931626 | 1.85E+08 | − | Island | |
| cg18206867 | 62422351 | − | Island | B3GNT2(tss1500) |
| cg23327896 | 73669290 | + | | DNAJB13(body) |
| cg13591848 | 1.03E+08 | − | Island | PDZD7(body); SFXN3(tss1500) |
| cg09368716 | 1361655 | + | Island | TMEM88B(body) |
| cg24187351 | 68525883 | − | Island | |
| cg07521193 | 27819422 | − | | |
| cg13105599 | 67356641 | − | N_Shore | |
| cg06155303 | 29460817 | − | Island | |
| cg26218577 | 69744466 | + | N_Shore | RPLP1(tss1500) |
| cg14433314 | 1233469 | − | | |
| cg21766667 | 48287888 | − | | |
| cg05868531 | 2.32E+08 | − | Island | |
| cg25337631 | 1.04E+08 | − | | |
| cg14372753 | 18064176 | − | Island | SLC25A18(body) |
| cg01721149 | 1.56E+08 | − | S_Shelf | C1orf61(body) |
| cg14802355 | 1.04E+08 | − | S_Shore | MANBA(tss1500) |
| cg13934406 | 32120878 | + | N_Shore | LOC100507547(body); PPT2(tss1500); |
| | | | | PPT2- |
| | | | | EGFL8(tss1500); PRRT1(tss1500) |
| cg09965419 | 30854186 | + | S_Shore | DDR1(body) |
| cg24853438 | 77139492 | + | N_Shore | |
| cg12408990 | 1.8E+08 | + | Island | RASGEF1C(body) |
| cg19760250 | 36997627 | + | Island | C17orf98(body) |
| cg22952142 | 68549178 | + | | |
| cg16216407 | 24797140 | + | N_Shelf | SOD3(body) |
| cg11905611 | 14720974 | − | Island | PLBD1(tss200) |
| cg09117688 | 41496623 | − | Island | SUGT1P3(tss1500); TPTE2P5(tss1500) |
| cg18192325 | 1.06E+08 | − | S_Shore | |
| cg21252282 | 90439702 | + | Island | |
| cg20286956 | 39547918 | − | Island | |
| cg03102494 | 1.48E+08 | + | | NBPF8(body) |
| cg16915863 | 54523294 | + | S_Shelf | LOC400043(body) |
| cg09428340 | 1.64E+08 | + | Island | PACRG(body) |
| cg11557492 | 56002240 | − | Island | SSC5D(body) |
| cg20580913 | 1599288 | + | Island | |
| cg05226558 | 46617183 | + | N_Shore | TDGF1(body) |
| cg09510531 | 45705742 | − | Island | AIRE(body) |
| cg17571919 | 28829328 | + | N_Shelf | LOC401242(body) |
| cg05224659 | 43295595 | − | S_Shore | |
| cg22861548 | 1.44E+08 | − | S_Shore | LYNX1(tss1500); LYNX1(tss200) |
| cg11617964 | 32118399 | − | Island | PRRT1(body) |
| cg02430039 | 47967818 | + | N_Shore | |
| cg14759977 | 41496782 | − | Island | SUGT1P3(tss1500); TPTE2P5(tss1500) |
| cg21182196 | 95786801 | − | S_Shore | CLMN(tss1500) |
| cg03188580 | 28829378 | + | N_Shelf | LOC401242(body) |
| cg19389372 | 36485356 | + | Island | SDHAF1(tss1500) |
| cg08123207 | 32118312 | − | Island | PRRT1(body) |
| cg15584071 | 36484913 | − | Island | SDHAF1(tss1500) |
| cg14459158 | 96720562 | + | N_Shore | |
| cg19579217 | 10720630 | − | N_Shelf | |
| cg08877188 | 1.96E+08 | + | | SLC51A(body) |
| cg22109795 | 2.1E+08 | + | S_Shelf | |
| cg03042692 | 23310188 | − | | |
| cg25661792 | 1.94E+08 | + | Island | |
| cg19328828 | 1.23E+08 | + | | HCAR1(tss1500) |
| cg04579398 | 36485360 | + | Island | SDHAF1(tss1500) |
| cg00611789 | 78985432 | + | | CMYA5(tss1500) |
| cg25460807 | 21908022 | − | S_Shelf | |
| cg07266144 | 1.32E+08 | + | Island | PDLIM4(body) |
| cg17378342 | 1.5E+08 | − | Island | CDX1(tss200) |
| cg06092502 | 42611687 | + | Island | |
| cg19962075 | 48256050 | + | Island | MAPK4(body) |
| cg10831246 | 18553663 | + | | |
| cg02247178 | 31506687 | + | | TEX26(tss200); TEX26-AS1(body) |
| cg01206378 | 95698827 | + | N_Shore | RWDD3(tss1500); TMEM56- |
| | | | | RWDD3(body) |
| cg03466525 | 50650021 | − | | |
| cg27438152 | 31515761 | − | | LOC283710(body) |
| cg10244666 | 32421808 | + | | WT1(body) |
| cg12914966 | 28830789 | − | N_Shore | LOC401242(body) |
| cg17445765 | 64403162 | + | | |
| cg06025456 | 32120863 | + | N_Shore | LOC100507547(body); PPT2(tss1500); |
| | | | | PPT2 |
| | | | | EGFL8(tss1500); PRRT1(tss1500) |
| cg24010336 | 39467258 | − | S_Shore | FBXO17(tss1500) |
| cg20909380 | 99987288 | − | | |
| cg25483741 | 1.44E+08 | − | S_Shore | LYNX1(tss1500) |
| cg08979191 | 1.32E+08 | + | S_Shore | SEPT8(tss1500); SEPT8(tss200) |
| cg12236088 | 1.05E+08 | + | Island | |
| cg08216684 | 77144016 | + | N_Shelf | |
| cg24070990 | 2.29E+08 | − | S_Shore | |
| cg12974138 | 59325950 | + | N_Shelf | |
| cg11669516 | 2.18E+08 | − | S_Shelf | IGFBP2(body) |
| cg09655403 | 78985495 | − | | CMYA5(tss200) |
| cg19978312 | 1.8E+08 | + | Island | RASGEF1C(body) |
| cg21475076 | 1100493 | + | Island | |
| cg05819154 | 49902436 | + | | |
| cg02452552 | 1.51E+08 | + | | |
| cg00083206 | 1.11E+08 | − | Island | DDO(body) |
| cg07429623 | 2.05E+08 | + | | KLHDC8A(body); KLHDC8A(tss200) |
| cg10663765 | 1.94E+08 | + | Island | |
| cg17330303 | 27189648 | + | N_Shore | SFN(body) |
| cg16306978 | 21266953 | + | Island | APOB(tss200) |
| cg03679504 | 46621556 | − | S_Shelf | TDGF1(body) |
| cg08289130 | 3210357 | − | S_Shore | |
| cg23180489 | 1.44E+08 | + | S_Shore | LYNX1(tss1500); LYNX1(tss200) |
| cg02783887 | 660856 | − | Island | |
| cg13392078 | 36036514 | − | N_Shore | RECK(tss1500) |
| cg17662083 | 36004632 | + | | DMKN(tss200) |
| cg06878548 | 514821 | − | | |
| cg24808901 | 95698954 | + | N_Shore | RWDD3(tss1500); TMEM56- |
| | | | | RWDD3(body) |
| cg25453677 | 1835735 | − | N_Shore | |
| cg08089567 | 45705994 | + | Island | AIRE(body) |
| cg07853425 | 1.34E+08 | + | | C5orf66(body) |
| cg20592017 | 40364740 | + | | |
| cg12231340 | 52685221 | + | Island | KRT81(body) |
| cg18361418 | 28499676 | + | | GPX5(body) |
| cg17493885 | 1.77E+08 | − | Island | NSD1(tss1500) |
| cg19264571 | 10454085 | + | Island | APCDD1(tss1500) |
| cg23279355 | 78985592 | − | | CMYA5(tss200) |
| cg14003022 | 3043019 | − | Island | |
| cg01791034 | 42328501 | − | | PLA2G4E(body) |
| cg22455128 | 25212486 | + | Island | |
| cg25665606 | 50986051 | + | Island | KLHDC7B(tss1500) |
| cg19227031 | 32118315 | − | Island | PRRT1(body) |
| cg03453449 | 95945120 | − | S_Shelf | USP44(body) |
| cg16484473 | 50649723 | − | | |
| cg20632887 | 1.6E+08 | − | | ATP1A2(body) |
| cg00399059 | 1.44E+08 | − | S_Shore | LYNX1(tss1500); LYNX1(tss200) |
| cg07168556 | 48912483 | − | | WFIKKN2(tss200) |
| cg21277452 | 28829340 | + | N_Shelf | LOC401242(body) |
| cg27179622 | 2.26E+08 | + | Island | LEFTY2(body) |
| cg00070899 | 34024479 | − | S_Shore | GRM4(body) |
| cg13058819 | 2.34E+08 | − | S_Shore | SLC35F3(body) |
| cg12407057 | 1366206 | + | S_Shelf | |
| cg07752120 | 70746259 | − | | |
| cg09013220 | 45944385 | − | | |
| cg10859192 | 54071672 | + | S_Shore | ATP5G2(tss1500) |
| cg19786602 | 7966326 | + | | |
| cg17910899 | 1E+08 | − | | |
| cg21334513 | 30095248 | − | Island | |
| cg12716639 | 1.19E+08 | + | N_Shore | USP2(body) |
| cg24024660 | 99195788 | + | Island | LOC100289187(tss200) |
| cg16361867 | 1.21E+08 | − | | GRIK4(body) |
| cg12182937 | 33215649 | + | N_Shore | |
| cg14906690 | 76573506 | + | Island | |
| cg00213295 | 32925561 | − | | TMEM132E(body) |
| cg06687305 | 46088486 | − | Island | CCDC17(body) |
| cg01119278 | 1.11E+08 | + | Island | DDO(body) |
| cg25356715 | 1904796 | + | N_Shore | CACNA2D4(body) |
| cg24874173 | 1.45E+08 | + | S_Shore | LOC100288142(body); NBPF10(body) |
| cg00107241 | 29461070 | − | Island | |
| cg16677448 | 36004699 | − | | DMKN(tss200) |
| cg04388919 | 1.49E+08 | − | N_Shore | |
| cg05745631 | 30095178 | − | Island | |
| cg02703145 | 93922525 | + | Island | |
| cg16087940 | 1.3E+08 | + | Island | |
| cg00481725 | 98648111 | + | | |
| cg12033622 | 30225573 | − | Island | COX4I2(tss200) |
| cg18228590 | 77142469 | + | S_Shore | |
| cg18700133 | 8013202 | − | Island | ALOXE3(body) |
| cg25642234 | 14721092 | + | Island | PLBD1(tss1500) |
| cg15920791 | 61835708 | − | Island | |
| cg13641713 | 1.39E+08 | − | N_Shore | |
| cg13734833 | 68779817 | + | S_Shore | MRGPRF(body) |
| cg03593471 | 10709415 | − | | |
| cg20889395 | 48912556 | + | | WFIKKN2(tss200) |
| cg12127196 | 50489240 | + | N_Shore | AGBL4(body) |
| cg21215576 | 1.24E+08 | − | N_Shore | FAM83A(body) |
| cg21386414 | 26421349 | + | | |
| cg04459939 | 90291141 | − | N_Shelf | |
| cg07191900 | 17564994 | − | Island | IL17RA(tss1500) |
| cg13842639 | 38488083 | + | Island | |
| cg07734975 | 9780844 | − | S_Shore | LOC440028(body); SBF2-AS1(body) |
| cg24002541 | 50649795 | + | | |
| cg14552982 | 1.34E+08 | − | N_Shore | EXOSC2(tss1500) |
| cg01395127 | 1601229 | + | N_Shore | |
| cg13202523 | 99588273 | + | | |
| cg04715503 | 3062795 | + | N_Shore | CLDN9(body) |
| cg08880849 | 40274728 | − | Island | HSPB9(tss200); KAT2A(tss1500) |
| cg20245362 | 1.28E+08 | − | N_Shore | |
| cg09373015 | 41338895 | + | N_Shore | |
| cg13702536 | 1.23E+08 | + | | HCAR1(tss1500) |
| cg22670033 | 1.81E+08 | − | N_Shore | |
| cg03342928 | 56002446 | − | Island | SSC5D(body) |
| cg06489965 | 3062975 | − | N_Shore | CLDN9(body) |
| cg27268279 | 71672083 | + | | COL13A1(body) |
| cg27532360 | 42452994 | − | | ITGA2B(body) |
| cg09814029 | 39212920 | − | | |
| cg02723291 | 22777465 | − | N_Shore | ETNK1(tss1500) |
| cg05293490 | 95156546 | + | | |
| cg03532263 | 1.03E+08 | − | S_Shore | |
| cg07349815 | 1.24E+08 | − | N_Shore | |
| cg24460126 | 1.8E+08 | − | Island | FAM163A(body) |
| cg14481208 | 74669375 | + | Island | RTKN(tss1500) |
| cg06555959 | 61835620 | − | Island | |
| cg12529273 | 54458246 | − | | LNX1(tss1500); LNX1-AS2(tss1500) |
| cg02179982 | 46365647 | + | N_Shore | WNT7B(body) |
| cg26776957 | 73357183 | + | | PLEKHB1(tss1500); PLEKHB1(tss200) |
| cg05912299 | 1E+08 | − | | |
| cg12094552 | 46449961 | + | S_Shore | |
| cg14947478 | 34025962 | − | S_Shelf | GDF5(body) |
| cg01207473 | 1.59E+08 | − | N_Shore | |
| cg14172108 | 34405553 | − | N_Shore | |
| cg21109038 | 90543610 | − | Island | ZNF710(tss1500) |
| cg01510388 | 65711658 | − | Island | CYP7B1(tss1500) |
| cg23193613 | 1.44E+08 | + | Island | |
| cg13411784 | 1.4E+08 | + | Island | DPH7(tss1500) |
| cg03677069 | 88718317 | + | | MMRN2(tss1500); SNCG(body) |
| cg04896168 | 86968543 | − | | |
| cg11069430 | 95945082 | + | S_Shelf | USP44(body) |
| cg01343363 | 58533808 | − | N_Shore | NDRG4(body); NDRG4(tss1500) |
| cg12229151 | 6340283 | + | N_Shore | PRKCDBP(body) |
| cg20481312 | 59157579 | + | Island | |
| cg09047034 | 38762714 | + | N_Shore | |
| cg04821248 | 86337418 | − | N_Shore | |
| cg13266496 | 1.11E+08 | − | N_Shore | DDO(body) |
| cg01607625 | 32847830 | + | Island | |
| cg01863042 | 23309892 | − | | |
| cg08610426 | 49249123 | + | | IZUMO1(body) |
| cg20595752 | 35458416 | − | S_Shelf | TEAD3(body) |
| cg13349472 | 25427451 | − | Island | |
| cg21687003 | 26806309 | + | | |
| cg10541110 | 39215894 | − | | KRTAP2-3(body) |
| cg11210878 | 62689065 | + | N_Shore | CHRM1(tss200) |
| cg01293485 | 42847044 | − | S_Shore | HIGD1A(tss1500) |
| cg18927077 | 1.04E+08 | − | Island | |
| cg24579589 | 2.35E+08 | + | | |
| cg06672093 | 1.04E+08 | − | S_Shelf | |
| cg12733396 | 40783234 | + | S_Shore | COL9A2(tss1500) |
| cg19860353 | 1.95E−08 | + | | |
| cg18890544 | 2.42E+08 | − | Island | |
| cg18959621 | 23310464 | − | | |
| cg25240254 | 56000944 | + | Island | SSC5D(body) |
| cg26884773 | 2.35E+08 | − | | |
| cg09938479 | 1.78E+08 | − | Island | |
| cg15602677 | 1166067 | − | S_Shelf | SNTG2(body) |
| cg19127638 | 70322442 | − | S_Shore | TET1(body) |
| cg07824824 | 12254976 | − | Island | CIDEA(body) |
| cg22031964 | 62677251 | − | | CHRM1(body) |
| cg13647973 | 56061337 | + | Island | |
| cg02904657 | 40571997 | + | N_Shore | |
| cg08274637 | 51417923 | + | Island | DLEU7(tss200); DLEU7-AS1(body) |
| cg09616559 | 25921151 | + | | |
| cg01887374 | 1.29E+08 | − | Island | HS6ST1(tss1500) |
| cg02299497 | 69095570 | + | S_Shore | |
| cg14625636 | 1E+08 | − | N_Shore | LOC100499484(tss1500); LOC100499484- |
| | | | | C9ORF174(tss1500) |
| cg03993109 | 1.79E−05 | + | Island | ADAMTS2(body) |
| cg09221960 | 1.2E+08 | − | N_Shore | GRIK4(body); GRIK4(tss1500) |
| cg01043616 | 50475035 | + | Island | ASIC1(body) |
| cg05413199 | 67430454 | − | S_Shore | ZDHHC1(body) |
| cg23173402 | 2.28E+08 | − | | |
| cg21123519 | 69095679 | + | S_Shore | |
| cg11070193 | 26527870 | − | | CATSPER4(body) |
| cg13176198 | 30336915 | + | Island | |
| cg18947553 | 2.28E+08 | − | Island | |
| cg11237406 | 4649333 | + | | ZMYND15(body) |
| cg17674042 | 34482479 | − | | PACSIN1(body); PACSIN1(tss200) |
| cg17112426 | 36004716 | − | | DMKN(tss200) |
| cg16503797 | 19476805 | − | N_Shore | |
| cg05593775 | 1.03E+08 | − | Island | PDZD7(body) |
| cg17512474 | 1.5E+08 | − | Island | CDX1(tss200) |
| cg03577139 | 24796988 | − | | SOD3(tss200) |
| cg04717895 | 31541298 | − | | |
| cg21148531 | 1.07E−08 | − | S_Shore | ATG5(tss1500) |
| cg08798685 | 27730294 | − | | LOC100131289(body) |
| cg05374238 | 57285466 | + | | C1orf168(tss200) |
| cg01906695 | 48399493 | + | S_Shore | COL2A1(tss1500) |
| cg17762073 | 34024220 | + | Island | GRM4(body) |
| cg07554053 | 44885910 | + | S_Shelf | |
| cg05067427 | 54227914 | − | N_Shore | |
| cg10442355 | 61326412 | + | | LOC100505532(tss200) |
| cg00326131 | 74668477 | − | Island | RTKN(body); RTKN(tss1500) |
| cg06410849 | 66955988 | − | Island | RRAD(body) |
| cg17356252 | 45705686 | − | Island | AIRE(tss200) |
| cg15006681 | 38199759 | − | N_Shore | H1F0(tss1500) |
| cg16447311 | 1604134 | − | N_Shore | |
| cg25360385 | 51786547 | − | S_Shore | GALNT6(tss1500); SLC4A8(body) |
| cg22623319 | 1.12E+08 | − | N_Shelf | RBM20(body) |
| cg19512582 | 61083516 | + | | |
| cg10408284 | 48912327 | − | | WFIKKN2(tss1500) |
| cg14742937 | 39172097 | + | | ADAM5(tss200) |
| cg02242734 | 18064224 | − | S_Shore | SLC25A18(body) |
| cg04664126 | 28554262 | + | N_Shore | SCAND3(body) |
| cg06184825 | 36772775 | + | | CPNE5(body) |
| cg04807108 | 78697933 | − | | |
| cg08461752 | 43522343 | + | | |
| cg05568941 | 38517913 | + | N_Shore | GJD3(body) |
| cg09978105 | 1.03E+08 | − | | |
| cg27446137 | 77723198 | + | Island | |
| cg07218647 | 1.21E+08 | + | S_Shelf | |
| cg07227033 | 12725802 | + | N_Shore | ARL4A(tss1500) |
| cg23305129 | 1.55E+08 | − | S_Shore | |
| cg04625615 | 41788368 | − | Island | ITPKA(body) |
| cg20955328 | 1.26E+08 | − | Island | KLF15(body) |
| cg13341668 | 50359909 | + | Island | HYAL2(body) |
| cg06420834 | 35169380 | − | Island | MYL9(tss1500) |
| cg13565129 | 28601419 | − | N_Shore | |
| cg19782271 | 1.72E+08 | + | S_Shore | STK10(tss1500) |
| cg02257791 | 22217085 | + | | |
| cg27537199 | 2885085 | + | Island | |
| cg00220225 | 50194470 | + | S_Shore | SEMA3F(body) |
| cg18137414 | 42313943 | + | N_Shore | |
| cg14190522 | 1.24E+08 | − | | DAB2IP(body) |
| cg06117341 | 3062653 | − | N_Shore | CLDN9(body) |
| cg02547167 | 8751687 | − | S_Shore | |
| cg03609493 | 11370314 | − | Island | MIR572(tss200) |
| cg17736526 | 1329387 | − | Island | |
| cg19873297 | 1.72E+08 | + | S_Shore | STK10(tss1500) |
| cg00668150 | 2615720 | + | S_Shore | CLUH(tss1500) |
| cg14478475 | 37905113 | − | S_Shore | SLC25A51(tss1500) |
| cg25415695 | 1.35E+08 | + | S_Shore | |
| cg14920044 | 91296311 | + | S_Shore | SLC16A12(tss1500) |
| cg22275125 | 62783573 | − | | SLC22A8(tss1500) |
| cg21793452 | 1.97E−08 | − | N_Shore | |
| cg15123755 | 40075810 | + | | |
| cg00610692 | 74668072 | + | Island | RTKN(body); RTKN(tss1500) |
| cg17052170 | 1.44E+08 | + | Island | LOC100133669(body); LY6E(tss1500) |
| cg17454857 | 34127524 | + | N_Shelf | |
| cg24176731 | 73357164 | − | | PLEKHB1(tss1500); PLEKHB1(tss200) |
| cg16667508 | 43936853 | − | S_Shore | MATN4(body); RBPJL(body) |
| cg00079785 | 48172764 | − | N_Shore | SPIDR(tss1500) |
| cg15701111 | 52685334 | − | S_Shore | KRT81(tss200) |
| cg11322879 | 74378838 | + | | |
| cg02896835 | 92012615 | − | Island | |
| cg16624888 | 1.1E+08 | − | | |
| cg18971434 | 28829398 | − | N_Shelf | LOC401242(body) |
| cg25432738 | 21009861 | + | Island | CRYL1(body) |
| cg15175162 | 15657657 | + | Island | FBXL5(tss1500) |
| cg05366561 | 30437731 | − | Island | |
| cg12043145 | 97205952 | − | N_Shore | |
| cg13207630 | 32358064 | + | | |
| cg18604823 | 56687364 | − | | GALP(tss200) |
| cg14644378 | 34988588 | + | N_Shore | DNAJB5(tss1500) |
| cg24496745 | 57285156 | + | | C1orf168(body) |
| cg00054210 | 65711644 | − | Island | CYP7B1(tss1500) |
| cg25001930 | 1.48E+08 | + | | NBPF8(body) |
| cg02920396 | 68782211 | + | S_Shelf | MRGPRF(tss1500) |
| cg04896115 | 47674905 | − | | |
| cg17886420 | 1796742 | − | | |
| cg11757040 | 73209188 | + | | DPF3(body) |
| cg00689340 | 74668672 | − | Island | RTKN(body); RTKN(tss1500) |
| cg17774102 | 28829640 | + | N_Shelf | LOC401242(body) |
| cg07298985 | 22133076 | − | Island | PIWIL2(body); PIWIL2(tss200) |
| cg25838465 | 92012736 | − | S_Shore | |
| cg06599908 | 54931632 | − | S_Shelf | TTYH1(body) |
| cg09113483 | 61517807 | − | N_Shore | |
| cg16034541 | 1.95E+08 | − | S_Shore | MUC4(body) |
| cg26644853 | 57406257 | + | | CX3CL1(tss200) |
| cg22350048 | 22580632 | − | | |
| cg03727500 | 2.32E+08 | + | Island | |
| cg05463589 | 88706426 | + | Island | IL17C(body) |
| cg09214243 | 29968124 | − | S_Shore | |
| cg17827650 | 1.57E+08 | + | Island | NOM1(tss1500) |
| cg02987388 | 46089045 | + | Island | CCDC17(body) |
| cg21160372 | 22369310 | + | N_Shore | NCAM2(tss1500) |
| cg05254747 | 22223937 | − | N_Shore | SLC39A14(tss1500) |
| cg15116481 | 51330265 | − | Island | KLK15(body) |
| cg00913360 | 1.52E+08 | − | | |
| cg13981310 | 1.4E+08 | − | S_Shore | ENTPD2(tss1500) |
| cg04566512 | 46457588 | + | Island | |
| cg25653641 | 28601312 | − | N_Shore | |
| cg18675097 | 28227127 | + | Island | NKAPL(body) |
| cg00772991 | 2.21E+08 | + | | |
| cg13655803 | 48912764 | + | | WFIKKN2(body) |
| cg17295878 | 77924665 | + | S_Shore | TBC1D16(body); TBC1D16(tss200) |
| cg23109344 | 496440 | + | S_Shore | TDRP(tss1500) |
| cg18919720 | 79495335 | − | Island | FSCN2(tss200) |
| cg02002217 | 24520348 | + | | ANKRD20A19P(body) |
| cg05485462 | 3062349 | + | N_Shore | CLDN9(tss200) |
| cg09240693 | 86038538 | + | Island | |
| cg03558837 | 2.39E+08 | − | | ESPNL(body) |
| cg13583664 | 1.32E+08 | + | | |
| cg25571880 | 2.33E+08 | − | N_Shelf | PRSS56(tss1500) |
| cg06106599 | 70048801 | + | N_Shore | FADD(tss1500) |
| cg09083345 | 1603532 | + | N_Shore | |
| cg02754470 | 59157857 | + | S_Shore | |
| cg24516106 | 72079413 | + | Island | |
| cg25556225 | 31515750 | − | | LOC283710(body) |
| cg09336899 | 2.34E+08 | + | Island | NGEF(body) |
| cg11737757 | 2885168 | − | Island | |
| cg15868235 | 12486114 | + | | |
| cg14972141 | 1E+08 | − | | |
| cg05415308 | 71406783 | − | N_Shore | CT62(body) |
| cg12813394 | 69095057 | + | Island | |
| cg17136073 | 23310675 | + | | |
| cg09820729 | 12039712 | − | N_Shore | MFN2(tss1500) |
| cg17681516 | 43295389 | − | Island | |
| cg00176638 | 2.03E+08 | − | N_Shore | KDM5B(tss1500) |
| cg21186576 | 79466077 | − | Island | |
| cg06813382 | 81967195 | + | Island | LINC00857(tss1500) |
| cg13655082 | 1.25E+08 | − | Island | |
| cg10504392 | 1.1E+08 | + | | |
| cg22717478 | 29542598 | − | Island | OVCH1-AS1(body) |
| cg09133892 | 61325879 | − | | LOC100505532(body) |
| cg06911613 | 85846184 | − | S_Shore | |
| cg02623028 | 23003569 | − | Island | |
| cg25942596 | 82355828 | − | | |
| cg00224807 | 2.38E+08 | + | | |
| cg08580014 | 1.18E+08 | + | Island | PNLIPRP1(body) |
| cg07412254 | 1.21E+08 | + | | LINC01101(body) |
| cg01337207 | 32063835 | + | Island | TNXB(body) |
| cg13640071 | 1.03E+08 | − | | TCEAL7(body) |
| cg27243166 | 64479496 | + | Island | NRXN2(body) |
| cg14563637 | 98931801 | − | | |
| cg20219329 | 1994478 | − | | MYOM2(body) |
| cg12414681 | 29968195 | − | S_Shore | |
| cg04206219 | 1602137 | + | Island | |
| cg24437523 | 1.6E+08 | − | | ATP1A2(body) |
| cg09203312 | 20805196 | + | N_Shore | GJB6(body) |
| cg12428514 | 5407119 | − | | UCN3(body) |
| cg13073141 | 1.51E+08 | − | S_Shore | CERS2(tss1500) |
| cg11343894 | 1.54E+08 | + | | S100A1(tss1500); S100A13(body); S100A13 |
| | | | | (tss200) |
| cg18081818 | 23246105 | − | Island | |
| cg09644356 | 1.21E+08 | + | | |
| cg00568384 | 1.74E+08 | − | | |
| cg20495964 | 36744880 | + | | CPNE5(body) |
| cg27508620 | 21209725 | − | | |
| cg21572914 | 28829332 | + | N_Shelf | LOC401242(body) |
| cg26575633 | 1.57E+08 | + | | NTRK1(body) |
| cg27211198 | 1.22E+08 | − | | |
| cg03619568 | 77139388 | − | N_Shore | |
| cg15478483 | 1.12E+08 | − | | |
| cg13142134 | 42695332 | + | Island | ATP6V0CP3(body) |
| cg03701930 | 1981436 | + | | |
| cg22022881 | 1.06E+08 | − | Island | |
| cg07499899 | 21966310 | − | | RAP1GAP(body) |
| cg01234610 | 48912265 | − | | WFIKKN2(tss1500) |
| cg03335125 | 46088541 | − | Island | CCDC17(body) |
| cg18976044 | 24520049 | + | | ANKRD20A19P(body) |
| cg00718036 | 1.09E+08 | − | N_Shore | SGMS2(tss1500) |
| cg04972348 | 1100035 | + | Island | |
| cg12487162 | 12845864 | − | | TEAD1(body) |
| cg21181453 | 1.31E+08 | − | S_Shore | DPM2(tss200) |
| cg18174881 | 45723353 | + | S_Shore | MIS18BP1(tss1500) |
| cg14736365 | 1.74E+08 | + | | |
| cg24690588 | 57285399 | − | | C1orf168(tss200) |
| cg05096492 | 28828946 | + | N_Shelf | LOC401242(body) |
| cg13588403 | 73209128 | + | | DPF3(body) |
| cg09187505 | 1.06E+08 | − | Island | |
| cg03583746 | 1604013 | − | N_Shore | |
| cg24911721 | 46508454 | + | | MIR4763(tss1500); MIRLET7A3(tss200); |
| | | | | MIRLET7B(tss1500); MIRLET7BHG |
| | | | | (body) |
| cg21544713 | 61326407 | + | | LOC100505532(tss200) |
| cg09355528 | 71712830 | + | | COL13A1(body) |
| cg22619549 | 1.02E+08 | − | | PKD2L1(tss200) |
| cg22816091 | 42611670 | + | Island | |
| cg22877452 | 61517876 | + | N_Shore | |
| cg25318971 | 55837483 | − | N_Shore | BMP7(body) |
| cg25937216 | 58172855 | + | Island | LOC286177(tss1500) |
| cg00937681 | 77723304 | + | Island | |
| cg27539527 | 1.57E+08 | − | Island | |
| cg06590074 | 691211 | + | S_Shore | |
| cg05866021 | 57285278 | + | | C1orf168(body) |
| cg13883721 | 1.32E+08 | − | | |
| cg02994923 | 1.21E+08 | + | | |
| cg20310608 | 25128805 | − | Island | LOC284798(body); LOC284798(tss200) |
| cg27399125 | 1.5E+08 | − | N_Shore | |
| cg26587870 | 27730563 | − | | LOC100131289(body) |
| cg19537511 | 8213813 | + | | ARHGEF15(body); ARHGEF15(tss1500) |
| cg11934832 | 45683841 | − | N_Shelf | |
| cg24657518 | 1599401 | + | Island | |
| cg04236639 | 34402565 | − | N_Shelf | |
| cg15644756 | 69198743 | − | N_Shelf | LOC100130075(body) |
| cg00934322 | 30854109 | + | S_Shore | DDR1(body) |
| cg06633543 | 1E+08 | − | S_Shore | CYP46A1(body) |
| cg05924882 | 40883034 | − | N_Shelf | |
| cg17906007 | 35944645 | − | N_Shelf | RASD2(body) |
| cg14686645 | 42452426 | + | | ITGA2B(body) |
| cg11883009 | 13198960 | + | Island | NFIX(body) |
| cg05991492 | 3988700 | − | N_Shore | |
| cg15720535 | 1.4E+08 | − | Island | AGPAT2(tss1500) |
| cg26314966 | 79466160 | + | Island | |
| cg20593826 | 59280370 | − | Island | LOC100131060(body) |
| cg24998527 | 42611607 | + | Island | |
| cg23638640 | 25160033 | + | Island | PIWIL3(body); TOP1P2(tss1500) |
| cg06203364 | 2137307 | + | Island | C1orf86(body) |
| cg07371589 | 1.51E+08 | + | Island | SEMA6C(body) |
| cg24696183 | 2891360 | + | S_Shore | KCNQ1DN(body) |
| cg02941741 | 2.34E+08 | + | Island | NGEF(body); NGEF(tss1500) |
| cg05215994 | 23310203 | − | | |
| cg23989336 | 79283915 | + | Island | LINC00482(tss1500) |
| cg26624732 | 70746330 | − | | |
| cg10082647 | 1.07E+08 | − | N_Shore | C12orf23(tss1500) |
| cg27520536 | 54228323 | − | Island | |
| cg26787381 | 25128777 | − | Island | LOC284798(body); LOC284798(tss200) |
| cg06188367 | 45359611 | + | N_Shore | TMEM72-AS1(body) |
| cg19659741 | 39172099 | + | | ADAM5(tss200) |
| cg25830307 | 77871644 | − | N_Shore | C10orf11(body) |
| cg18524788 | 25890808 | + | N_Shore | |
| cg13870494 | 72658358 | + | N_Shore | MAMDC2(tss200) |
| cg21117965 | 2.2E+08 | − | | SPEG(body); SPEG(tss200) |
| cg05897163 | 37782302 | + | N_Shore | PPP1R1B(tss1500) |
| cg24795825 | 79164541 | − | N_Shore | MORF4L1(tss1500) |
| cg02049180 | 1.57E+08 | − | N_Shore | INSRR(body); NTRK1(body) |
| cg02873315 | 1.32E+08 | + | Island | |
| cg22316093 | 45246308 | + | Island | |
| cg04308185 | 38084377 | − | S_Shore | ORMDL3(tss1500) |
| cg14453762 | 1.35E+08 | + | | NTNG2(body) |
| cg16726706 | 1.17E+08 | − | | |
| cg12883279 | 32120773 | + | N_Shore | LOC100507547(body); PPT2(tss1500); |
| | | | | PPT2- |
| | | | | EGFL8(tss1500); PRRT1(tss1500) |
| cg04149015 | 31159854 | − | Island | PRSS36(body) |
| cg00307818 | 55463592 | − | S_Shore | BSND(tss1500) |
| cg01191259 | 29210460 | − | | |
| cg04196143 | 1.62E+08 | + | Island | |
| cg23381646 | 44187310 | − | S_Shore | GCK(body) |
| cg05752349 | 1.75E+08 | − | | |
| cg20368818 | 33800056 | + | S_Shelf | |
| cg26874229 | 1.06E+08 | + | Island | |
| cg05656990 | 23310635 | + | | |
| cg01803461 | 86604522 | − | S_Shore | |
| cg12208770 | 1361666 | + | Island | TMEM88B(body) |
| cg10730435 | 99987955 | − | | |
| cg23652484 | 2.12E+08 | + | | |
| cg04478991 | 1.96E+08 | + | | SLC51A(tss200) |
| cg16151651 | 1609119 | | | |
| cg19566586 | 1.81E+08 | + | Island | TRIM7(body) |
| cg19185846 | 71123233 | − | N_Shelf | |
| cg01971181 | 1.59E+08 | + | N_Shore | MFSD1(tss1500) |
| cg05189127 | 1.27E+08 | − | | |
| cg17598339 | 1.71E+08 | − | Island | |
| cg25426302 | 32120826 | − | N_Shore | LOC100507547(body); PPT2(tss1500); |
| | | | | PPT2- |
| | | | | EGFL8(tss1500); PRRT1(tss1500) |
| cg06640630 | 2.12E+08 | − | N_Shore | RD3(body) |
| cg09407967 | 41375649 | + | N_Shore | |
| cg23669081 | 46685353 | − | Island | HOXB7(body) |
| cg23458168 | 30864867 | + | N_Shore | ZNF536(body) |
| cg07355189 | 45923374 | + | Island | ALOX5(body) |
| cg20167074 | 1.52E+08 | − | S_Shore | S100A10(tss1500) |
| cg21490444 | 1.71E+08 | − | | |
| cg09285851 | 10099656 | − | | |
| cg01874730 | 57406425 | − | | CX3CL1(body) |
| cg05771342 | 68782202 | − | S_Shelf | MRGPRF(tss1500) |
| cg22953865 | 99988654 | + | | CCDC85C(body) |
| cg04058281 | 980580 | − | | |
| cg24729928 | 31480184 | − | S_Shore | FAM60A(tss1500) |
| cg04154027 | 78985588 | − | | CMYA5(tss200) |
| cg14875327 | 1.97E+08 | + | | |
| cg01826254 | 87609929 | + | | |
| cg06307176 | 1.31E+08 | − | Island | LOC728637(body) |
| cg06320582 | 87047010 | + | | |
| cg16501323 | 45705618 | − | Island | AIRE(tss200) |
| cg22972858 | 1.23E+08 | + | | HCAR1(tss200) |
| cg20055237 | 2.4E+08 | − | | |
| cg20638675 | 45374906 | − | | TMEM72-AS1(body) |
| cg20893180 | 36957379 | − | | |
| cg06294700 | 64330959 | + | N_Shore | |
| cg14389669 | 1.36E+08 | − | Island | ADAMTSL2(body); ADAMTSL2(tss1500) |
| cg20091636 | 61326482 | + | | LOC100505532(tss200) |
| cg04387835 | 4649076 | + | | ZMYND15(body) |
| cg26939946 | 19314395 | + | | RNF112(tss200) |
| cg14677130 | 38445916 | − | | EGFLAM(body) |
| cg27359374 | 1.58E+08 | + | | |
| cg04589210 | 86038589 | − | Island | |
| cg14035368 | 1.39E+08 | + | Island | CARD9(body); DNLZ(tss1500) |
| cg21160842 | 32112091 | + | N_Shore | RCN1(tss1500) |
| cg23058037 | 1.59E+08 | − | Island | VIPR2(body) |
| cg15491131 | 24914324 | − | N_Shore | |
| cg09088406 | 27819306 | + | | |
| cg24698211 | 1.71E+08 | + | N_Shore | FGF18(body) |
| cg16926213 | 1841314 | − | S_Shore | |
| cg12082025 | 1064218 | − | Island | ABCA7(body) |
| cg24280832 | 77871958 | − | N_Shore | C10orf11(body) |
| cg11801727 | 2.29E+08 | + | Island | |
| cg04270401 | 1.03E+08 | + | N_Shore | |
| cg17611235 | 1991188 | − | Island | |
| cg20201270 | 45397874 | + | Island | |
| cg03704673 | 4692237 | − | Island | GLTPD2(body) |
| cg11027707 | 49967669 | − | S_Shore | MON1A(tss1500) |
| cg05527459 | 92012148 | − | N_Shore | |
| cg23389215 | 64375765 | + | Island | NRXN2(body) |
| cg20035032 | 1820533 | + | | |
| cg07290269 | 31479853 | − | S_Shore | FAM60A(tss1500) |
| cg14711997 | 1.51E+08 | + | Island | KCNH2(body) |
| cg19299265 | 1975631 | − | | |
| cg00426822 | 39546536 | − | N_Shore | |
| cg21587238 | 42953590 | − | S_Shore | |
| cg13474370 | 65068885 | − | S_Shore | RBPMS2(tss1500) |
| cg10282491 | 3062368 | − | N_Shore | CLDN9(tss200) |
| cg14971320 | 43699070 | + | S_Shore | RASGEF1A(body) |
| cg21969792 | 1594995 | − | N_Shore | |
| cg05150608 | 1.03E+08 | − | | |
| cg08090164 | 1.44E+05 | + | S_Shore | LYNX1(tss1500); LYNX1(tss200) |
| cg10444806 | 38084428 | + | S_Shore | ORMDL3(tss1500) |
| cg06618740 | 1100126 | + | Island | |
| cg15282281 | 88024557 | + | S_Shore | GRID1(body); MIR346(tss200) |
| cg13187827 | 31734192 | − | | VWA7(body) |
| cg10932486 | 61028265 | + | | |
| cg15090036 | 48706075 | − | | |
| cg01057945 | 50303785 | − | | LOC283332(body) |
| cg10022914 | 1.02E+08 | − | N_Shore | |
| cg10321156 | 63687223 | + | S_Shore | |
| cg14381677 | 2.03E+08 | + | N_Shore | KDM5B(tss1500); PCAT6(tss1500) |
| cg22171758 | 54146133 | + | S_Shore | CISTR-ACT(body); CISTR- |
| | | | | ACT(tss1500) |
| cg22657492 | 1.38E+08 | − | Island | GFRA3(body) |
| cg15234946 | 1.17E+08 | + | | |
| cg07264025 | 86658860 | + | | MGAT4C(body) |
| cg13451356 | 40598648 | − | Island | |
| cg25701444 | 54521977 | − | S_Shore | LOC400043(body) |
| cg16205352 | 49688176 | + | N_Shore | PRPH(tss1500) |
| cg02845274 | 48494362 | + | Island | ABCA13(body) |
| cg23243408 | 11049855 | + | N_Shelf | |
| cg13136220 | 48100957 | + | S_Shore | LOC100287846(body) |
| cg23444264 | 72219751 | + | S_Shore | |
| cg01015662 | 40598394 | − | N_Shore | |
| cg18877667 | 48051089 | − | S_Shore | DLX4(body) |
| cg02797195 | 43185674 | + | N_Shore | RIPK4(body) |
| cg06939851 | 2.26E+08 | − | Island | PYCR2(tss1500) |
| cg27152312 | 96221849 | + | | |
| cg24361265 | 44068668 | + | Island | ELL3(body); SERF2(tss1500) |
| cg00049595 | 41650636 | + | N_Shore | |
| cg24980657 | 1.5E+08 | + | S_Shore | |
| cg23683010 | 3254065 | − | Island | MRGPRE(ts1500) |
| cg17158913 | 10764886 | + | Island | CASZ1(body) |
| cg04797061 | 31159920 | + | Island | PRSS36(body) |
| cg24025550 | 59157523 | − | Island | |
| cg05131488 | 14031822 | − | S_Shore | ETV1(tss1500) |
| cg13521002 | 22642322 | − | | PEBP4(body) |
| cg25862444 | 1.48E+08 | − | Island | NBPF8(body) |
| cg27283514 | 86768117 | − | | |
| cg00780971 | 1.49E+08 | − | | |
| cg01515960 | 2494587 | + | Island | |
| cg23038338 | 46996546 | − | N_Shelf | |
| cg01162093 | 1.34E+08 | − | | |
| cg02992639 | 1.55E+08 | + | Island | |
| cg06136160 | 1.64E+08 | + | Island | PACRG(body) |
| cg01271726 | 26323586 | − | | |
| cg19021188 | 1.44E+08 | + | S_Shore | LYNX1(tss1500); LYNX1(tss200) |
| cg16674433 | 19314408 | + | | RNF112(tss200) |
| cg12262698 | 45704988 | − | N_Shore | FAM118A(tss1500); FAM118A(tss200) |
| cg03954144 | 20751679 | + | Island | |
| cg00433296 | 22223852 | + | N_Shore | SLC39A14(tss1500) |
| cg14635466 | 1.02E+08 | + | N_Shore | SNX31(body) |
| cg23005885 | 90543450 | + | Island | ZNF710(tss1500) |
| cg21049587 | 46938148 | + | Island | RGN(body) |
| cg19041857 | 27730383 | − | | LOC100131289(body) |
| cg19529709 | | | | |
| | 25160075 | + | Island | PIWIL3(body); TOP1P2(tss1500) |
| cg23080060 | 61028303 | + | | |
| cg00424451 | 76338660 | − | S_Shore | |
| cg07786675 | 27189985 | + | Island | SFN(body) |
| cg05310240 | 47979069 | + | N_Shore | |
| cg13419925 | 1.59E+08 | − | N_Shore | MFSD1(tss1500) |
| cg03329019 | 2.21E+08 | − | N_Shore | |
| cg12165772 | 48070354 | + | N_Shore | DLX3(body) |
| cg24346776 | 1.48E+08 | − | Island | NBPF8(body) |
| cg11750580 | 46088336 | + | Island | CCDC17(body) |
| cg20768358 | 3574999 | − | | TP73(body) |
| cg12080906 | 1.57E+08 | + | N_Shore | NES(body) |
| cg20395040 | 23310225 | + | | |
| cg20660989 | 35150136 | + | Island | |
| cg17542176 | 24390246 | − | | |
| cg19401111 | 1.96E+08 | + | | SLC51A(tss200) |
| cg06853455 | 1.14E+08 | + | | |
| cg08269485 | 1769382 | + | Island | IFITM10(body); MOB2(body) |
| cg26633084 | 56660328 | − | S_Shore | |
| cg07318204 | 1.46E+08 | − | Island | HHIP(tss1500); HHIP-AS1(body) |
| cg26675395 | 31159623 | + | Island | PRSS36(body) |
| cg03136493 | 1.67E+08 | + | | |
| cg04066637 | 8619943 | − | Island | TMEM114(body) |
| cg18026197 | 1.26E+08 | + | N_Shore | CHCHD6(tss1500) |
| cg14218447 | 1.13E+08 | − | S_Shelf | |
| cg05190790 | 15672856 | + | S_Shore | |
| cg13818316 | 43185803 | + | N_Shore | RIPK4(body) |
| cg16060369 | 43698834 | − | S_Shore | RASGEF1A(body) |
| cg11324650 | 1.94E+08 | + | Island | |
| cg21287054 | 54928012 | + | Island | TTYH1(body) |
| cg02770672 | 1.64E+08 | − | | |
| cg15122439 | 2E+08 | + | S_Shore | |
| cg14074251 | 2.2E+08 | − | N_Shore | SPEG(tss1500) |
| cg22532774 | 2.39E+08 | − | N_Shore | ESPNL(body) |
| cg02959988 | 40075124 | − | | |
| cg03455316 | 62516405 | − | Island | |
| cg16837557 | 1.26E+08 | − | | KIRREL3(body) |
| cg09517766 | 44894102 | − | | |
| cg27516159 | 1904847 | − | N_Shore | CACNA2D4(body) |
| cg05565052 | 38669495 | + | S_Shore | TMEM184B(tss1500) |
| cg13306087 | 71713491 | + | Island | |
| cg00870514 | 29717946 | − | N_Shore | RAB11FIP4(tss1500) |
| cg05090759 | 1.66E+08 | + | | SLC38A11(tss200) |
| cg24176311 | 1.29E+08 | + | Island | TNPO3(tss1500); TPI1P2(body) |
| cg13837679 | 44329915 | − | N_Shore | ALX4(body) |
| cg01587386 | 65970924 | − | Island | |
| cg00816387 | 1975591 | + | | |
| cg03110795 | 1329410 | + | Island | |
| cg15670924 | 73669256 | + | | DNAJB13(body) |
| cg03222009 | 1.29E+08 | − | Island | HS6ST1(tss1500) |
| cg16330552 | 44010115 | + | | |
| cg04457658 | 37732313 | + | | |
| cg02954987 | 49170599 | + | | LAMB2(body) |
| cg13466284 | 27189679 | + | N_Shore | SFN(body) |
| cg01628467 | 7037406 | + | | |
| cg05092353 | 1.14E+08 | + | | F10(tss200) |
| cg03743678 | 47048004 | − | Island | |
| cg13001878 | 1.14E+08 | − | | F10(tss200) |
| cg03287432 | 1.35E+08 | + | | |
| cg22400703 | 68545995 | + | N_Shore | CNRIP1(body) |
| cg27637303 | 1.19E+08 | − | N_Shore | |
| cg07142285 | 57042703 | − | Island | APCDD1L(body) |
| cg11942181 | 1.3E+08 | + | Island | |
| cg08515989 | 29912904 | + | Island | ASPHD1(body) |
| cg01412970 | 17109239 | − | Island | PLD6(body) |
| cg13170076 | 382356 | − | | |
| cg07743117 | 77146278 | − | N_Shore | |
| cg14659662 | 54151053 | + | | GLIS1(body) |
| cg19573236 | 65733388 | + | Island | |
| cg04134279 | 496327 | − | S_Shore | TDRP(tss1500) |
| cg22715761 | 88428147 | − | | LDB3(tss1500); LDB3(tss200) |
| cg08023416 | 1.03E+08 | + | Island | IL1RL2(body) |
| cg13270055 | 36960499 | − | Island | CACNG2(body) |
| cg00346985 | 46940516 | − | S_Shore | |
| cg21903286 | 42876128 | + | N_Shore | PRICKLE1(body) |
| cg19867917 | 3642629 | − | Island | COLEC11(body) |
| cg24356964 | 3605979 | + | N_Shore | TP73(body); TP73(tss1500) |
| cg16893614 | 1.11E+08 | + | S_Shore | ALX3(tss1500) |
| cg02362978 | 9550137 | − | S_Shore | USP43(body) |
| cg06893273 | 46604774 | + | Island | |
| cg17535961 | 1.71E+08 | + | | |
| cg02952809 | 1.28E+08 | − | N_Shore | HILPDA(tss1500) |
| cg11609571 | 1.45E+08 | − | S_Shore | PRELID2(tss1500) |
| cg13680337 | 71626580 | + | | COL13A1(body) |
| cg08575722 | 32421845 | + | | WT1(body) |
| cg22270027 | 81128234 | − | Island | |
| cg24830730 | 1.51E+08 | + | N_Shore | KCNH2(body); KCNH2(tss1500) |
| cg01071644 | 1.39E+08 | + | N_Shelf | QSOX2(tss1500) |
| cg04585209 | 6292311 | − | Island | CCKBR(body) |
| cg11288801 | 1406661 | − | N_Shore | DAZAP1(tss1500) |
| cg04204221 | 1.21E+08 | + | N_Shore | |
| cg21167269 | 98981500 | + | Island | |
| cg04057238 | 71713446 | − | Island | |
| cg25433552 | 1.6E+08 | − | | |
| cg14748380 | 1.8E+08 | − | Island | FAM163A(body) |
| cg08710469 | 1.57E+08 | − | | |
| cg14301190 | 31159704 | + | Island | PRSS36(body) |
| cg08579577 | 8751402 | + | Island | |
| cg24524629 | 39203621 | + | | KRTAP2-1(tss200) |
| cg07045469 | 48494579 | − | Island | ABCA13(body) |
| cg11342437 | 47126328 | + | N_Shore | LRCH1(tss1500) |
| cg20421058 | 7337225 | + | | SORCS2(body) |
| cg18900591 | 50419193 | + | S_Shore | SALL4(tss200) |
| cg04413853 | 58499706 | + | S_Shore | C17orf64(tss200) |
| cg12550344 | 1.51E+08 | + | | |
| cg02331910 | 1.54E+08 | + | | S100A1(tss1500); S100A13(body); S100A13(tss1500); |
| | | | | S100A13(tss200) |
| cg25716013 | 75954053 | − | | COX7A2(tss1500) |
| cg20275928 | 79283959 | + | Island | LINC00482(tss1500) |
| cg09366730 | 2341370 | − | Island | ZFYVE28(body) |
| cg27532171 | 46952054 | − | S_Shore | |
| cg06943627 | 47590179 | + | Island | LOC100288866(body); NGFR(body) |
| cg21191514 | 2187921 | − | Island | TH(body) |
| cg21363811 | 1.8E+08 | + | Island | FAM163A(body) |
| cg00039463 | 3931489 | − | Island | CREBBP(tss1500) |
| cg12634306 | 40098811 | − | | HEYL(body) |
| cg12865939 | 8832938 | + | | ST5(body); ST5(tss1500); ST5(tss200) |
| cg14425869 | 98343728 | + | | |
| cg22902089 | 46318223 | + | S_Shore | CREB3L1(body) |
| cg19153828 | 1.28E+08 | − | Island | |
| cg01205935 | 99737585 | + | N_Shore | DOCK9(body); DOCK9- |
| | | | | AS2(tss1500) |
| cg09100338 | 48676054 | − | N_Shore | |
| cg15015539 | 2.2E+08 | − | N_Shore | DES(tss200) |
| cg17017404 | 44329245 | − | N_Shore | ALX4(body) |
| cg00802903 | 1.56E+08 | − | N_Shelf | |
| cg23006204 | 1.31E+08 | − | S_Shore | DPM2(tss200) |
| cg08801479 | 32165200 | − | S_Shore | NOTCH4(body) |
| cg23414861 | 74064064 | + | Island | DPPA5(tss200) |
| cg19286437 | 88428117 | + | | LDB3(tss1500); LDB3(tss200) |
| cg02085507 | 6739192 | − | N_Shore | TRIP10(tss1500) |
| cg23290482 | 36997720 | − | S_Shore | C17orf98(tss200) |
| cg23533513 | 76849324 | − | Island | MYO7A(body) |
| cg25731401 | 10572392 | + | Island | |
| cg13753527 | 37040454 | + | S_Shelf | |
| cg04406111 | 1.24E+08 | − | | TACC2(body) |
| cg01713950 | 17766995 | + | S_Shore | RCC2(tss1500) |
| cg12443001 | 2467281 | + | S_Shore | |
| cg12516097 | 17051629 | − | Island | |
| cg08893853 | 1.52E+08 | + | | RIIAD1(body) |
| cg25567448 | 97175065 | + | S_Shore | NEURL3(tss1500) |
| cg05118960 | 50359978 | + | Island | HYAL2(body) |
| cg08991643 | 1.94E+08 | + | Island | |
| cg20597877 | 24882099 | − | Island | |
| cg09527615 | 94883350 | + | Island | |
| cg25439883 | 26323563 | − | | |
| cg07085632 | 1.26E+08 | − | | |
| cg25317157 | 1.28E+08 | − | | C10orf90(body) |
| cg05124918 | 52473462 | − | Island | |
| cg22175814 | 71739339 | + | | LOC100134391(body) |
| cg19721065 | 1.14E+08 | + | Island | RASAL1(body) |
| cg17851725 | 1.21E+08 | + | | |
| cg25607216 | 1.27E+08 | − | | |
| cg14618634 | 89193057 | − | | |
| cg13793718 | 25212248 | + | N_Shore | |
| cg05324407 | 86987828 | − | | |
| cg15247509 | 48416977 | + | S_Shelf | GDF2(tss200) |
| cg07140497 | 1.14E+08 | + | Island | RASAL1(body) |
| cg16208084 | 40274703 | − | Island | HSPB9(tss200); KAT2A(tss1500) |
| cg24129222 | 74643251 | + | Island | C2orf81(body) |
| cg19829856 | 44237658 | − | S_Shore | LOXHD1(tss1500) |
| cg11512608 | 73357276 | + | | PLEKHB1(body); PLEKHB1(tss1500) |
| cg14269301 | 19532166 | − | N_Shore | MAP3K15(body) |
| cg21923770 | 1183159 | + | | TUSC5(body) |
| cg19815818 | 7037365 | + | | |
| cg08404221 | 39523309 | + | S_Shore | FBXO27(tss200) |
| cg23678210 | 36391852 | + | S_Shore | PXT1(body) |
| cg23951474 | 2188061 | + | Island | TH(body) |
| cg19679397 | 70048852 | − | N_Shore | FADD(tss1500) |
| cg01568228 | 90439543 | + | N_Shore | |
| cg13198297 | 18046564 | + | | MYO15A(body) |
| cg21771200 | 11784761 | − | N_Shore | ZNF833P(tss200) |
| cg06230839 | 43189892 | − | S_Shelf | |
| cg02110835 | 71953083 | − | S_Shelf | |
| cg18750087 | 43522143 | − | | |
| cg23236370 | 33892705 | + | S_Shore | ADAMTS12(tss1500) |
| cg26145670 | 28554497 | + | N_Shore | SCAND3(body) |
| cg14560110 | 20799470 | + | Island | CCDC144NL(tss200); LOC440416(body) |
| cg11345143 | 20989349 | − | Island | CRYL1(body) |
| cg14614094 | 1.34E+08 | + | N_Shore | EXOSC2(tss1500) |
| cg08804111 | 1.03E+08 | − | | |
| cg19265480 | 1.48E+08 | − | N_Shore | NBPF8(body) |
| cg03292743 | 74642838 | − | Island | C2orf81(body) |
| cg09266924 | 29034669 | + | Island | LOC646278(tss1500) |
| cg20066226 | 1E+08 | − | | |
| cg05931423 | 1.16E+08 | + | Island | |
| cg08435674 | 36308494 | + | | |
| cg04117972 | 2.28E+08 | + | | |
| cg06347454 | 48494519 | − | Island | ABCA13(body) |
| cg26242687 | 41471014 | − | N_Shore | |
| cg14654363 | 28601443 | − | N_Shore | |
| cg05332854 | 54228582 | − | S_Shore | |
| cg12754571 | 2.48E+08 | − | Island | GCSAML(body); GCSAML- |
| | | | | AS1(tss200); OR2C3(body) |
| cg00380835 | 51165752 | − | Island | SHANK1(body) |
| cg17482114 | 1.23E+08 | + | | ADCY5(body) |
| cg03309252 | 98314632 | − | N_Shore | |
| cg17279652 | 1.81E+08 | − | Island | TRIM7(body) |
| cg26715042 | 49681307 | + | | CRISP2(tss200) |
| cg08895590 | 91227319 | − | | |
| cg20011248 | 2.28E+08 | + | Island | |
| cg11911679 | 1.83E+08 | + | N_Shore | CERKL(body) |
| cg21436055 | 2.28E+08 | − | N_Shore | |
| cg07225641 | 46618325 | + | Island | TDGF1(body); TDGF1(tss1500) |
| cg05927817 | 18020357 | − | | |
| cg12243133 | 66823616 | + | N_Shore | RHOD(tss1500) |
| cg08592890 | 30907246 | + | S_Shore | CTF1(tss1500) |
| cg03215599 | 1598921 | + | N_Shore | |
| cg21198712 | 98534327 | − | | |
| cg11644585 | 8958019 | − | | NTN1(body) |
| cg22878388 | 1.06E+08 | + | Island | |
| cg26645709 | 89987792 | + | | |
| cg24942922 | 1.2E+08 | − | N_Shore | |
| cg05934682 | 50394298 | + | S_Shore | IL4I1(body) |
| cg20457732 | 71172486 | − | | |
| cg15587476 | 24397810 | − | Island | FAM228A(tss200) |
| cg10551329 | 32120933 | + | N_Shore | LOC100507547(body); PPT2(tss1500); |
| | | | | PPT2- |
| | | | | EGFL8(tss1500); PRRT1(tss1500) |
| cg20736031 | 1.14E+08 | + | | |
| cg17465304 | 1.17E+08 | + | Island | KIF12(tss200) |
| cg04357965 | 77945347 | + | S_Shore | LHFPL2(tss1500) |
| cg13110574 | 46493169 | + | N_Shore | ADARB1(tss1500); SSR4P1 (tss200) |
| cg22302929 | 28554373 | − | N_Shore | SCAND3(body) |
| cg18184769 | 12039714 | + | N_Shore | MFN2(tss1500) |
| cg23869158 | 47126023 | − | N_Shore | LRCH1(tss1500) |
| cg16155393 | 46617303 | − | N_Shore | TDGF1(body) |
| cg17277199 | 24397845 | − | Island | FAM228A(tss200) |
| cg01194320 | 50493166 | − | | |
| cg25138412 | 2.29E+08 | − | S_Shore | |
| cg06259612 | 1.33E+08 | − | N_Shore | |
| cg07751331 | 26205865 | + | S_Shore | KIF3C(tss1500) |
| cg26175343 | 6034888 | + | S_Shore | LRRN4(tss200) |
| cg04981619 | 43328122 | + | Island | |
| cg10755878 | 1.67E+08 | + | | |
| cg12934258 | 1.59E+08 | + | | SYTL3(body) |
| cg14565151 | 95945384 | − | S_Shelf | USP44(tss200) |
| cg03359095 | 61002866 | − | | RBBP8NL(tss1500) |
| cg25937577 | 31536476 | − | | PLA2G3(tss200) |
| cg09243648 | 45944464 | + | | |
| cg06210630 | 1.6E+08 | − | | ATP1A2(tss200) |
| cg09547692 | 91027278 | + | | |
| cg25520960 | 2.2E+08 | + | N_Shelf | |
| cg20871559 | 1.56E+08 | + | S_Shelf | |
| cg19739906 | 1.39E+08 | − | | HNMT(body) |
| cg05618807 | 14376820 | + | Island | |
| cg09669754 | 78166148 | − | S_Shore | CARD14(body) |
| cg04177826 | 50985681 | + | Island | KLHDC7B(tss1500) |
| cg13064789 | 1.54E+08 | − | | |
| cg08234664 | 49170568 | + | | LAMB2(tss200) |
| cg00349061 | 26371865 | − | N_Shore | SLC30A2(body) |
| cg23694533 | 40806193 | + | S_Shore | |
| cg08571918 | 1.05E+08 | + | Island | |
| cg08133631 | 26527909 | − | | CATSPER4(body) |
| cg21929564 | 42757171 | − | | CCDC152(body) |
| cg16913064 | 45949878 | − | Island | |
| cg06829154 | 1.3E+08 | − | Island | |
| cg11704490 | 1.62E+08 | − | S_Shore | |
| cg26337070 | 85999873 | − | | ATOH8(body) |
| cg27111444 | 54590808 | + | | |
| cg15078284 | 36004994 | − | | DMKN(tss1500) |
| cg05560575 | 1.17E+08 | − | | |
| cg12193833 | 30244370 | + | Island | |
| cg07506749 | 54228447 | + | Island | |
| cg25866552 | 55072298 | − | Island | |
| cg26907313 | 71167117 | − | | ATP6V1B1(body) |
| cg23378165 | 9550256 | + | S_Shore | USP43(body) |
| cg21852429 | 36997740 | + | S_Shore | C17orf98(tss200) |
| cg09374627 | 97206170 | + | N_Shore | |
| cg17149103 | 1.48E+08 | + | N_Shore | NBPF8(body) |
| cg18098722 | 556889 | − | S_Shore | C11orf35(body) |
| cg09264065 | 29460936 | + | Island | |
| cg10563109 | 5087749 | + | S_Shore | |
| cg02698580 | 86016643 | + | | RGR(body) |
| cg10775231 | 81967666 | − | S_Shore | LINC00857(body) |
| cg26173906 | 14376389 | + | N_Shore | |
| cg07524919 | 32063901 | + | Island | TNXB(body) |
| cg12388541 | 1.69E+08 | − | S_Shelf | |
| cg08457421 | 31536431 | − | | PLA2G3(body) |
| cg24727290 | 30850868 | − | N_Shore | DDR1(tss1500) |
| cg01517680 | 49499006 | − | Island | |
| cg02399371 | 79306875 | + | | |
| cg00216693 | 1.32E+08 | − | | |
| cg20328456 | 32121113 | − | N_Shore | LOC100507547(body); PPT2(tss1500); |
| | | | | PPT2(tss200); PPT2- |
| | | | | EGFL8(tss1500); PRRT1(tss1500) |
| cg01297627 | 55874586 | − | S_Shore | FAM71E2(body) |
| cg26827653 | 1.25E+08 | − | S_Shore | FEZ1(tss1500) |
| cg12150256 | 50359849 | + | Island | HYAL2(body) |
| cg21187770 | 26205876 | + | S_Shore | KIF3C(tss1500) |
| cg11455444 | 1.21E+08 | − | | LINC01101(tss200) |
| cg12560020 | 93823645 | − | | |
| cg07833467 | 50986511 | + | Island | KLHDC7B(body) |
| cg07313064 | 55072617 | + | Island | |
| cg20498637 | 41439456 | − | S_Shore | |
| cg00639615 | 89786496 | + | N_Shore | FANCI(tss1500) |
| cg22697684 | 9550173 | + | S_Shore | USP43(body) |
| cg18364858 | 1.03E+08 | + | | |
| cg25582488 | 69095171 | + | Island | |
| cg26207380 | 1.44E+08 | + | | |
| cg13752184 | 1E+08 | − | | |
| cg26817145 | 71807797 | + | | THSD4(body) |
| cg20559189 | 8101907 | + | N_Shore | TUB(body); TUB(tss1500) |
| cg23009780 | 96774492 | + | | |
| cg08951638 | 1.42E+08 | + | S_Shore | |
| cg02992311 | 39523626 | + | S_Shore | FBXO27(tss1500) |
| cg11724147 | 27703436 | + | S_Shore | HIBADH(tss1500) |
| cg01090821 | 2.39E+08 | + | N_Shore | |
| cg01987536 | 96890120 | + | N_Shore | AK7(body) |
| cg26630791 | 83017000 | + | N_Shore | HAPLN1(tss200) |
| cg21205855 | 61563866 | − | N_Shore | |
| cg08731696 | 59280489 | + | Island | LOC100131060(body) |
| cg07945733 | 1.18E+08 | + | Island | C10orf82(tss200) |
| cg25921357 | 55887905 | + | N_Shore | TMEM190(tss1500) |
| cg00320094 | 68695183 | + | N_Shore | WLS(body) |
| cg26873880 | 1.6E+08 | + | | |
| cg12948059 | 1.3E+08 | + | Island | |
| cg27009812 | 3062597 | + | N_Shore | CLDN9(body) |
| cg14887505 | 1258581 | + | | |
| cg07223180 | 20989142 | − | Island | CRYL1(body) |
| cg08951271 | 30850543 | − | N_Shore | DDR1(tss1500) |
| cg23693749 | 76399434 | − | S_Shore | |
| cg08436104 | 38168076 | − | | |
| cg04637704 | 1.29E+08 | + | Island | TNPO3(tss1500); TPI1P2(body) |
| cg13245228 | 1.44E+08 | − | S_Shore | |
| cg16083044 | 1.69E+08 | + | | |
| cg21204522 | 27730016 | + | | LOC100131289(body) |
| cg02630477 | 40388223 | − | | |
| cg14904725 | 1.14E+08 | + | | F10(tss200) |
| cg10537450 | 1.48E+08 | − | Island | NBPF8(body) |
| cg15093383 | 1820445 | + | | |
| cg02566259 | 3043199 | − | Island | |
| cg16415457 | 23713441 | − | | |
| cg07083902 | 1596254 | − | N_Shelf | |
| cg00632007 | 1598984 | − | N_Shore | |
| cg26321999 | 30854011 | + | S_Shore | DDR1(body) |
| cg20449659 | 32421752 | − | | WT1(body) |
| cg24648385 | 88583845 | + | Island | |
| cg11124420 | 95675274 | − | N_Shore | |
| cg21850518 | 42357520 | − | | |
| cg21658372 | 49176968 | + | S_Shelf | NTN5(tss1500); SEC1P(body) |
| cg20415053 | 26527928 | − | | CATSPER4(body) |
| cg16059991 | 99975470 | − | | |
| cg25037000 | 2931557 | − | S_Shelf | |
| cg16268165 | 1.76E+08 | − | | UNC5A(body) |
| cg10075506 | 1817351 | + | | MYT1L(body) |
| cg00291837 | 55460884 | + | N_Shore | |
| cg25122233 | 54412506 | − | N_Shore | HOXC4(body); HOXC5(body); HOXC6 |
| | | | | (body) |
| cg00760562 | 1.96E+08 | − | | SLC51A(tss1500) |
| cg24833819 | 42924552 | − | Island | |
| cg10517650 | 1.13E+08 | + | S_Shore | SPICE1(tss1500) |
| cg25618731 | 2848409 | − | N_Shore | PRSS41(tss200) |
| cg03662997 | 15963027 | − | Island | |
| cg00597076 | 1395880 | + | S_Shore | MYO1C(body); MYO1C(tss1500) |
| cg26046136 | 45526613 | + | S_Shelf | EYA2(body) |
| cg21687740 | 1.57E+08 | − | Island | |
| cg02881684 | 45978153 | − | S_Shore | FOSB(body) |
| cg11550547 | 77734490 | + | | KCTD14(body); KCTD14(tss200); NDUFC2- |
| | | | | KCTD14(body) |
| cg16800939 | 1.01E+08 | − | | |
| cg19484305 | 1.24E+08 | + | | |
| cg22842797 | 48912621 | − | | WFIKKN2(body) |
| cg15526213 | 43818881 | − | S_Shore | |
| cg03357999 | 55516724 | − | N_Shore | |
| cg21025349 | 1.26E+08 | − | | KIRREL3(body) |
| cg04322572 | 43447518 | + | | |
| cg19510698 | 1.01E+08 | − | S_Shore | ALDH1A3(body) |
| cg02086195 | 12656232 | + | Island | DHRS3(body) |
| cg19907915 | 1.34E+08 | + | | IGSF98(body) |
| cg11631063 | 1.85E+08 | + | S_Shore | |
| cg25035376 | 57943261 | − | N_Shore | KIF5A(tss1500) |
| cg18348654 | 19476864 | − | Island | |
| cg23042086 | 1.02E+08 | − | Island | |
| cg21217540 | 1.11E+08 | − | Island | KCNC4(tss1500) |
| cg19137107 | 44541999 | + | S_Shore | |
| cg14379965 | 1.5E+08 | + | Island | |
| cg24652615 | 44243304 | − | Island | TMEM1518(body) |
| cg02193146 | 1.11E+08 | − | Island | KCNC4(tss1500) |
| cg18861753 | 1.78E+08 | + | N_Shore | COL23A1(body) |
| cg24129115 | 23352488 | + | N_Shelf | REM2(body) |
| cg19526353 | 42760742 | − | S_Shore | ERF(tss1500) |
| cg05115664 | 90621637 | + | Island | |
| cg18140268 | 1.48E+08 | + | N_Shore | NBPF8(body) |
| cg11206167 | 42924367 | − | Island | |
| cg05105069 | 1.03E+08 | − | | TCEAL7(body) |
| cg26331343 | 1.57E+08 | − | S_Shore | LINC00880(body); LINC00881(tss200) |
| cg05865340 | 2.4E+08 | − | | |
| cg15127250 | 51786489 | + | S_Shore | GALNT6(tss1500); SLC4A8(body) |
| cg19731612 | 1.77E+08 | − | Island | NSD1(tss1500) |
| cg01308968 | 58061859 | − | Island | IGFBP7-AS1(body) |
| cg15006627 | 13045938 | + | S_Shore | GPRC5A(body) |
| cg09069900 | 1.14E+08 | − | S_Shelf | F10(tss200) |
| cg03123541 | 95699097 | + | N_Shore | RWDD3(tss1500); TMEM56- |
| | | | | RWDD3(body) |
| cg27143204 | 54530375 | + | S_Shore | CCNO(tss1500) |
| cg02124912 | 2.02E+08 | − | | GPR37L1(tss200) |
| cg04218880 | 40388264 | + | | |
| cg25836232 | 12306837 | + | N_Shore | TUBB6(tss1500) |
| cg04182912 | 95157037 | − | | |
| cg25221984 | 1.46E+08 | − | Island | CD160(body); LOC100288142(body); |
| | | | | NBPF10(body) |
| cg03425348 | 20441979 | − | | |
| cg01668653 | 35733710 | − | Island | |
| cg17113856 | 32120895 | − | N_Shore | LOC100507547(body); PPT2(tss1500); |
| | | | | PPT2- |
| | | | | EGFL8(tss1500); PRRT1(tss1500) |
| cg17072519 | 21322063 | + | S_Shelf | AIFM3(body) |
| cg12043200 | 21090833 | − | N_Shore | |
| cg01781662 | 1.32E4-08 | + | N_Shore | NTMT1(body); NTMT1(tss1500) |
| cg09395805 | 27186076 | − | | |
| cg15166039 | 1.6E+08 | + | | |
| cg03676636 | 99064102 | − | N_Shore | STPG2(body) |
| cg08785317 | 86604854 | − | S_Shelf | |
| cg18786420 | 1.73E+08 | + | S_Shore | |
| cg08605301 | 29924135 | + | Island | WIPF3(body) |
| cg06705862 | 1.28E+08 | − | N_Shore | |
| cg13048437 | 94360096 | − | | |
| cg15448220 | 1.51E+08 | + | N_Shore | SETDB1(tss1500) |
| cg01231141 | 1.79E+08 | + | | ADAMTS2(body) |
| cg10612560 | 8019912 | − | N_Shelf | ALOXE3(body) |
| cg23323671 | 26233623 | + | Island | MIR3917(tss1500); STMN1(tss1500) |
| cg25488547 | 85303309 | − | | CHM(tss1500) |
| cg | | | | |
| 16665310 | 1E+08 | − | | |
| cg25454270 | 1.17E+08 | + | S_Shore | APOA5(body) |
| cg04578397 | 56650508 | − | N_Shore | MT1L(tss1500) |
| cg12497374 | 14339534 | − | Island | |
| cg17900861 | 99987322 | + | | |
| cg11374335 | 34971543 | − | N_Shore | WTIP(tss1500) |
| cg01902605 | 78366076 | + | S_Shore | BHMT2(body); DMGDH(tss1500) |
| cg14016257 | 1.86E+08 | + | N_Shore | |
| cg27651238 | 52644264 | − | Island | |
| cg06167719 | 38362727 | + | Island | HLCS(tss200) |
| cg07669403 | 19477059 | − | Island | |
| cg15753394 | 17282781 | − | S_Shore | RBM24(body); RBM24(tss200) |
| cg27295595 | 29796756 | + | Island | |
| cg06388647 | 1.21E+08 | − | | |
| cg09113474 | 1601369 | − | N_Shore | |
| cg25263801 | 1.13E+08 | + | | GPR85(body); GPR85(tss1500) |
| cg09835878 | 74815131 | − | | VRTN(tss200) |
| cg18033770 | 1.39E+08 | + | Island | CARD9(body); DNLZ(tss1500) |
| cg02760164 | 42371967 | − | S_Shore | PLA2G4D(body) |
| cg15255455 | 4534986 | + | N_Shore | PLINS(body) |
| cg18574813 | 72381308 | + | | PDE2A(body); PDE2A(tss1500) |
| cg26230275 | 1.52E+08 | − | N_Shore | S100A10(body) |
| cg04488758 | 95945366 | − | S_Shelf | USP44(tss200) |
| cg10665961 | 8958039 | − | | NTN1(body) |
| cg07090083 | 22642638 | + | | PEBP4(body) |
| cg15191795 | 8727474 | + | | |
| cg15752292 | 39970821 | − | N_Shore | TIMM50(tss1500) |
| cg14232218 | 93922330 | + | Island | |
| cg18346398 | 1615992 | + | S_Shore | |
| cg06893296 | 30095136 | − | N_Shore | |
| cg13430552 | 25427652 | − | S_Shore | |
| cg08641963 | 44451973 | − | N_Shore | TNNC2(body) |
| cg18973628 | 94245350 | − | N_Shore | LOC643037(tss1500) |
| cg15829969 | 1.48E+08 | − | | NBPF8(body) |
| cg22012583 | 1.51E+08 | + | Island | CERS2(tss1500) |
| cg16653408 | 42313569 | − | N_Shore | |
| cg00424169 | 31506837 | + | | TEX26(body); TEX26-AS1(tss200) |
| cg05501509 | 75789529 | − | Island | |
| cg01278041 | 1.84E+08 | − | N_Shore | WWC2-AS2(body) |
| cg14794735 | 1.29E+05 | + | N_Shelf | MIR183(tss1500); MIR96(tss1500) |
| cg16897193 | 46443801 | − | Island | NOVA2(body) |
| cg21053539 | 28829503 | + | N_Shelf | LOC401242(body) |
| cg02282631 | 42953543 | + | S_Shore | |
| cg14219918 | 1.27E+08 | − | Island | NR5A1(body) |
| cg08409225 | 56617737 | + | N_Shore | NABP2(tss1500) |
| cg17220161 | 2.03E+08 | − | Island | |
| cg19489705 | 77146291 | − | N_Shore | |
| cg01568522 | 51786038 | − | S_Shore | GALNT6(tss1500); SLC4A8(body) |
| cg03765423 | 1.94E+08 | − | Island | |
| cg06491573 | 1.26E+08 | − | Island | KLF15(body) |
| cg06846259 | 25384654 | − | Island | POMC(body) |
| cg05003599 | 1E+08 | − | S_Shelf | FBXO24(body); PCOLCE-AS1(body) |
| cg02570888 | 2.26E+08 | + | N_Shore | |
| cg14336501 | 35424059 | + | | |
| cg20068058 | 46317859 | − | S_Shore | CREB3L1(body) |
| cg04981056 | 1.96E+08 | + | | SLC51A(body) |
| cg20427865 | 57406353 | + | | CX3CL1(tss200) |
| cg19754520 | 1.02E+08 | − | Island | CPN1(body) |
| cg15414773 | 1100360 | + | Island | |
| cg17995050 | 55073248 | + | Island | |
| cg25969992 | 1.21E+08 | + | | CABP1(body) |
| cg07718903 | 6104261 | + | S_Shore | FERMT1(tss200) |
| cg00880984 | 82958011 | + | S_Shore | IBTK(tss1500) |
| cg14295062 | 1.4E+08 | + | S_Shore | ENTPD2(tss1500) |
| cg24168538 | 35527016 | + | | |
| cg09362608 | 6659805 | + | N_Shore | |
| cg00509187 | 44362093 | − | S_Shore | ENOX1(tss1500) |
| cg08873792 | 32847811 | − | Island | |
| cg20200200 | 18807388 | + | N_Shore | KLHDC7A(tss200) |
| cg05403241 | 1.22E+08 | + | | |
| cg23251248 | 55907374 | − | Island | TBPL2(tss200) |
| cg01652244 | 74063982 | + | Island | DPPA5(body) |
| cg19959917 | 44878373 | − | N_Shore | CXCL12(body) |
| cg07608867 | 49879854 | + | | |
| cg21790715 | 1.22E+08 | + | | |
| cg07189222 | 32847527 | − | Island | |
| cg08327367 | 46934663 | + | | LRP4(body) |
| cg12751521 | 1.1E+08 | − | | |
| cg02007567 | 10912696 | + | Island | TVP23A(tss200) |
| cg13663855 | 21095414 | + | S_Shelf | |
| cg15076318 | 32847788 | − | Island | |
| cg14624145 | 37024169 | − | N_Shore | |
| cg15865827 | 23713838 | − | Island | PSMA8(body) |
| cg11203293 | 25777762 | + | | |
| cg09361966 | 2.42E+08 | − | Island | |
| cg20112159 | 80907159 | + | | PTPRQ(body) |
| cg14334161 | 72201622 | − | S_Shore | NODAL(tss200) |
| cg17105755 | 74875317 | + | Island | M1AP(body); M1AP(tss200) |
| cg07870378 | 4665298 | − | Island | UBALD1(tss1500) |
| cg23897302 | 1.03E+08 | + | N_Shore | AMN(body) |
| cg17870909 | 47566968 | − | S_Shore | FTCD(body) |
| cg15083775 | 48054048 | − | S_Shelf | |
| cg13374701 | 27190088 | − | Island | SFN(body) |
| cg08597839 | 29461008 | + | Island | |
| cg16347256 | 57285160 | + | | C1orf168(body) |
| cg26302230 | 2.2E+08 | + | N_Shelf | WNT6(body) |
| cg13808012 | 2.44E+08 | + | Island | LOC339529(body) |
| cg01164574 | 22368940 | + | N_Shore | |
| cg10923408 | 31506710 | + | | TEX26(tss200); TEX26-AS1(body) |
| cg06520296 | 37350981 | − | | |
| cg03080147 | 17270347 | − | N_Shore | MTMR7(body) |
| cg24146100 | 99737448 | − | N_Shore | DOCK9(body) |
| cg05822532 | 73442531 | − | | ELN(body) |
| cg25078026 | 28829931 | + | N_Shore | LOC401242(body) |
| cg14981253 | 5112642 | − | S_Shore | RBAK- |
| | | | | RBAKDN(body); RBAKDN(body) |
| cg17816637 | 21387058 | − | S_Shore | SLC7A4(tss1500) |
| cg13802736 | 1.58E+08 | + | N_Shore | |
| cg07207652 | 45798257 | − | | |
| cg06714761 | 2.01E+08 | − | | |
| cg19593741 | 31734217 | + | | VWA7(body) |
| cg15671083 | 5406959 | − | | UCN3(tss200) |
| cg05998486 | 1.51E+08 | − | S_Shore | KCNH2(body) |
| cg26802194 | 57916919 | − | N_Shore | CNGB1(body) |
| cg03604322 | 32439052 | + | Island | SLC5A1(body) |
| cg21535947 | 25875133 | − | Island | NUPL1(tss1500) |
| cg11092486 | 5087604 | + | S_Shore | |
| cg25224330 | 5278293 | − | | |
| cg14244636 | 1E+08 | + | | |
| cg05950212 | 1.5E+08 | − | S_Shore | HIST2H2AB(tss1500) |
| cg10424681 | 4079350 | − | Island | C6orf201(tss200); FAM217A(body) |
| cg05135729 | 1.18E+08 | + | Island | |
| cg20270653 | 70962720 | − | | WBSCR17(body) |
| cg05631230 | 1.17E+08 | + | | |
| cg17091577 | 30854233 | + | S_Shore | DDR1(body) |
| cg06634862 | 54457645 | − | | LNX1(body); LNX1-AS2(tss1500) |
| cg00872984 | 32063991 | + | Island | TNXB(body) |
| cg13305951 | 19314618 | + | | RNF112(body) |
| cg02910281 | 39407451 | − | | RHBDL2(body) |
| cg19969694 | 41185800 | + | N_Shore | VPS18(tss1500) |
| cg26266427 | 32063838 | + | Island | TNXB(body) |
| cg19676181 | 56660398 | + | S_Shore | |
| cg14575854 | 37896175 | + | | GRB7(body); GRB7(tss200) |
| cg20118840 | 1.2E+08 | − | | |
| cg08614082 | 1.58E+08 | − | | |
| cg25123895 | 21267113 | − | S_Shore | APOB(tss200) |
| cg27431761 | 8361327 | + | | |
| cg15851278 | 73669449 | − | | DNAJB13(body) |
| cg13888070 | 1.7E+08 | + | N_Shore | LOC100128164(body); SEC62(tss1500) |
| cg15124926 | 1.03E+08 | − | | WTAPP1(body) |
| cg15620114 | 91296457 | + | S_Shore | SLC16A12(tss1500) |
| cg13939809 | 61669940 | − | Island | |
| cg19503249 | 2.48E+08 | + | | |
| cg26684363 | 1.4E+08 | + | Island | |
| cg25924274 | 1.21E+08 | + | | |
| cg20860806 | 49267825 | − | N_Shore | |
| cg02696067 | 30850977 | − | N_Shore | DDR1(tss1500) |
| cg08560952 | 50278816 | − | N_Shore | |
| cg17889682 | 95402733 | + | S_Shore | DYNC1I1(body) |
| cg14518658 | 37726676 | + | N_Shelf | |
| cg06390079 | 19648855 | + | S_Shore | ALDH3A1(body); ALDH3A1(tss200) |
| cg14938587 | 1.56E+08 | + | | |
| cg11521079 | 78955599 | + | S_Shore | |
| cg08867399 | 1.29E+08 | − | Island | HS6ST1(tss1500) |
| cg03146625 | 54448729 | + | S_Shore | HOXC4(body) |
| cg03381704 | 30565638 | + | | LINC00189(tss200) |
| cg21024112 | 41376993 | + | Island | |
| cg11632685 | 25128770 | − | Island | LOC284798(body); LOC284798(tss200) |
| cg16783571 | 74064594 | + | S_Shore | DPPA5(tss1500) |
| cg09062638 | 23003490 | + | Island | |
| cg09735896 | 39019978 | − | Island | KRT12(body) |
| cg18882670 | 1.18E+08 | − | Island | |
| cg18762806 | 48676000 | + | N_Shore | |
| cg00290607 | 67383545 | − | Island | DOC2GP(tss1500) |
| cg10376827 | 88730324 | + | N_Shore | ADIRF(body); AGAP11(tss200) |
| cg06856687 | 44934509 | + | N_Shore | CDH22(body) |
| cg16419584 | 1.3E+08 | + | Island | |
| cg22215678 | 19191718 | − | N_Shore | SNRPD1(tss1500) |
| cg11830800 | 41087520 | − | S_Shore | CNTN1(body) |
| cg08918020 | 30225706 | + | Island | COX412(body) |
| cg21583668 | 49967678 | − | S_Shore | MON1A(tss1500) |
| cg27539986 | 6664103 | − | S_Shore | KLHL21(tss1500) |
| cg23779495 | 28829674 | + | N_Shelf | LOC401242(body) |
| cg00050402 | 55073022 | + | Island | |
| cg02358323 | 44541718 | + | Island | |
| cg12905085 | 32054510 | + | Island | |
| cg06340713 | 85303427 | − | | CHM(tss1500) |
| cg07939626 | 30853073 | + | S_Shore | DDR1(body) |
| cg03125400 | 43327937 | − | Island | |
| cg19140429 | 48858746 | + | Island | |
| cg21683069 | 1.48E+08 | − | N_Shore | NBPF8(body) |
| cg24601559 | 43328536 | + | S_Shore | |
| cg22343877 | 1.76E+08 | − | | |
| cg01300914 | 2763797 | + | N_Shore | |
| cg03792783 | 1.21E+08 | + | S_Shelf | |
| cg01501896 | 40274670 | + | Island | HSPB9(tss200); KAT2A(tss1500) |
| cg25428553 | 1.05E+08 | − | Island | |
| cg21854759 | 92012499 | + | Island | |
| cg12860635 | 30095140 | − | N_Shore | |
| cg13290149 | 61002739 | + | | RBBP8NL(tss200) |
| cg05563558 | 3445716 | + | | |
| cg24591835 | 76942048 | − | S_Shore | |
| cg18322510 | 2.37E+08 | − | N_Shore | LGALS8(body); LGALS8(tss1500); LGALS8 |
| | | | | (tss200); LGALS8-AS1(body) |
| cg10825234 | 1041061 | − | N_Shelf | |
| cg23433008 | 1.8E+08 | + | Island | RASGEF1C(body) |
| cg01628528 | 24882594 | − | Island | C1QTNF9(tss1500) |
| cg20964304 | 1.25E+08 | + | S_Shelf | PKNOX2(body) |
| cg22382854 | 56124169 | + | | |
| cg08166631 | 40599011 | + | Island | |
| cg23926439 | 2.29E+08 | − | Island | |
| cg00088688 | 1.68E+08 | + | | |
| cg17329164 | 32121259 | − | N_Shore | LOC100507547(body); PPT2(body); |
| | | | | PPT2(tss1500); PPT2- |
| | | | | EGFL8(tss1500) |
| cg16510654 | 16330625 | − | | C1orf64(tss200) |
| cg02962952 | 75789439 | + | Island | |
| cg12526471 | 1.14E+08 | + | N_Shore | ZBTB16(tss1500) |
| cg18630644 | 4079528 | + | S_Shore | C6orf201(body); FAM217A(tss200) |
| cg11598935 | 55837619 | + | N_Shore | BMP7(body) |
| cg19421125 | 6882856 | + | Island | LAG3(body) |
| cg08863620 | 88136768 | + | Island | |
| cg06238004 | 42029727 | − | N_Shore | |
| cg07671036 | 46875766 | − | Island | PRSS42(tss200) |
| cg03951833 | 1.48E+08 | − | | NBPFS(body) |
| cg04160749 | 58172571 | + | Island | LOC286177(tss1500) |
| cg14950193 | 89873797 | + | N_Shore | C7orf63(tss1500) |
| cg08930131 | 1.78E+08 | − | | COL23A1(body) |
| cg03829542 | 67029374 | + | | |
| cg03253303 | 29753081 | − | | RAB11FIP4(body) |
| cg15656686 | 30854551 | + | S_Shore | DDR1(body) |
| cg14409029 | 19138243 | + | N_Shore | 2DHHC13(tss1500) |
| cg13881108 | 55073432 | + | Island | |
| cg16613240 | 10099623 | − | | |
| cg14221766 | 36818572 | + | | |
| cg00659878 | 2.3E+08 | + | | |
| cg23536158 | 48172746 | − | N_Shore | SPIDR(tss1500) |
| cg23415434 | 1.32E+08 | + | S_Shore | ARHGEF4(body) |
| cg10587449 | 2654874 | + | S_Shore | |
| cg22620027 | 21824391 | + | | |
| cg11359720 | 45246441 | + | Island | |
| cg24642820 | 13462465 | − | S_Shore | NUP210(tss1500) |
| cg10197057 | 42612518 | + | S_Shore | |
| cg16495975 | 8619855 | + | Island | TMEM114(body) |
| cg26227310 | 1.03E+08 | − | S_Shore | TLX1NB(body) |
| cg18210365 | 65066710 | + | N_Shore | RBPMS2(body) |
| cg13356540 | 7449057 | + | | CAMTA1(body) |
| cg10382206 | 2.09E+08 | − | | |
| cg21038703 | 42248358 | + | | ASB16(body) |
| cg23661466 | 63657173 | + | | |
| cg02610813 | 99067317 | − | N_Shelf | |
| cg00961617 | 37782380 | + | N_Shore | PPP1R1B(tss1500) |
| cg19154438 | 45825730 | + | N_Shore | CKM(body) |
| cg13688770 | 2367137 | + | S_Shore | ZFYVE28(body); ZFYVE28(tss1500) |
| cg10140454 | 17626019 | + | Island | RAI1(body) |
| cg27625401 | 3148536 | − | | ZSCAN10(body) |
| cg17565967 | 61002711 | + | | RBBP8NL(tss200) |
| cg22849655 | 32118375 | − | Island | PRRT1(body) |
| cg16370398 | 54448913 | + | S_Shore | HOXC4(body) |
| cg20509119 | 39378476 | − | | |
| cg17393016 | 79283390 | + | N_Shore | LINC00482(tss1500) |
| cg27422857 | 1.06E+08 | − | Island | |
| cg03557725 | 96251912 | + | N_Shore | SNRPF(tss1500) |
| cg24661236 | 36930062 | − | Island | PI16(body) |
| cg21813538 | 49967713 | − | S_Shore | MON1A(tss1500) |
| cg23737190 | 36930021 | − | Island | PI16(body) |
| cg00487142 | 1.09E+08 | + | | DPPA4(body) |
| cg00813720 | 1.54E+08 | − | N_Shore | |
| cg26279372 | 1.01E+08 | − | S_Shore | BEGAIN(tss200) |
| cg04720348 | 72922873 | + | S_Shore | ARHGEF28(body) |
| cg09810089 | 2.12E+08 | + | Island | RD3(body) |
| cg16530165 | 2.33E+08 | − | S_Shore | NPPC(tss1500) |
| cg19558933 | 1.35E+08 | − | Island | |
| cg14676272 | 49836160 | + | Island | |
| cg16358155 | 1.95E+08 | − | S_Shore | MUC4(body) |
| cg06840491 | 48416966 | − | S_Shelf | GDF2(tss200) |
| cg27170141 | 10711044 | − | N_Shelf | CASZ1(body) |
| cg26364871 | 1889757 | + | | |
| cg08198488 | 63333792 | + | N_Shore | TPM1(tss1500) |
| cg00099295 | 12597720 | − | N_Shore | MKRN2(tss1500) |
| cg09974661 | 3642634 | + | Island | COLEC11(body) |
| cg21545013 | 8407891 | − | S_Shelf | |
| cg00386405 | 30863275 | − | N_Shelf | ZNF536(tss200) |
| cg00296018 | 1.41E+08 | − | | |
| cg04123507 | 52695270 | + | N_Shore | KRT86(tss1500) |
| cg13686059 | 11101549 | − | S_Shore | |
| cg15196156 | 40388395 | − | | |
| cg07491676 | 76400783 | + | | C15orf27(body) |
| cg07357827 | 12200835 | + | S_Shore | SYN2(body); TIMP4(body) |
| cg22343728 | 46287028 | + | S_Shore | |
| cg09760963 | 1.55E+08 | − | Island | RNF175(body) |
| cg16215084 | 30853948 | − | S_Shore | DDR1(body) |
| cg06454084 | 47158242 | + | | DACT3(body) |
| cg00355514 | 24914782 | − | Island | |
| cg22500876 | 45187902 | − | | |
| cg03253268 | 44592883 | + | Island | CRYAA(body) |
| cg13946767 | 1.54E+08 | − | | S100A1(tss1500); S100A13(body); S100A13 |
| | | | | (tss200) |
| cg15574301 | 1.32E+08 | + | Island | PDLIM4(body) |
| cg03540028 | 43183977 | + | N_Shelf | RIPK4(body) |
| cg13705830 | 89518557 | + | | |
| cg27305460 | 29969096 | − | S_Shore | |
| cg15946312 | 1.81E+08 | − | Island | |
| cg25953930 | 41462541 | + | | |
| cg08450021 | 1.11E+08 | − | | |
| cg16968885 | 1.04E+08 | − | | COL11A1(tss1500) |
| cg08328513 | 78365691 | + | Island | BHMT2(body); DMGDH(tss200) |
| cg04863005 | 59043208 | + | Island | TACSTD2(tss200) |
| cg05148717 | 28829171 | − | N_Shelf | LOC401242(body) |
| cg00590063 | 17918795 | + | Island | B3GNT3(body) |
| cg18269141 | 2063966 | − | Island | PRKCZ(body) |
| cg02947450 | 22766861 | − | Island | |
| cg27561907 | 2848830 | − | Island | PRSS41(body) |
| cg12845268 | 63657363 | + | | |
| cg07703976 | 48494734 | + | Island | ABCA13(body) |
| cg27387888 | 19724444 | + | Island | |
| cg05573767 | 1.67E+08 | + | | RPS6KA2(body) |
| cg05903289 | 1.3E+08 | + | Island | |
| cg09313482 | 1.21E+08 | − | | |
| cg03139204 | 46937658 | + | Island | RGN(tss200) |
| cg05955675 | 85206653 | − | S_Shore | |
| cg01825213 | 98979965 | − | N_Shore | |
| cg11906444 | 43858174 | − | | CD177(body) |
| cg25390787 | 49681299 | + | | CRISP2(body) |
| cg05606376 | 38446192 | + | | EGFLAM(body) |
| cg15665630 | 1.26E+08 | − | N_Shore | |
| cg17792740 | 88729315 | + | N_Shore | ADIRF(body); AGAP11(tss1500) |
| cg11097968 | 1.03E+08 | + | S_Shore | |
| cg00589002 | 3606082 | + | N_Shore | TP73(body); TP73(tss1500) |
| cg13569486 | 8290738 | − | S_Shore | LMO1(tss1500) |
| cg05213635 | 1.29E+08 | − | S_Shore | |
| cg02066331 | 17282778 | − | S_Shore | RBM24(body); RBM24(tss200) |
| cg27597505 | 54148217 | − | S_Shelf | CISTR-ACT(body) |
| cg01185080 | 90543813 | + | Island | ZNF710(tss1500) |
| cg04139465 | 51020297 | − | Island | LRRC4B(body) |
| cg14223001 | 44978881 | + | Island | |
| cg07107113 | 16087752 | + | S_Shore | FBLIM1(body) |
| cg06850787 | 716357 | − | | |
| cg23629166 | 2345368 | + | N_Shore | PEX10(tss1500) |
| cg13159361 | 27819341 | − | | |
| cg24786658 | 1.13E+08 | − | | GPR85(body); GPR85(tss1500) |
| cg16391327 | 38746048 | + | N_Shore | FAM98B(tss1500) |
| cg06647332 | 1.31E+08 | − | Island | LOC728637(body) |
| cg00540400 | 79124168 | + | Island | |
| cg15398400 | 1.18E+08 | + | Island | C10orf82(body) |
| cg18582362 | 89975159 | + | N_Shore | GTPBP10(tss1500) |
| cg03850936 | 1.68E+08 | + | | |
| cg04774597 | 69744390 | + | N_Shore | RPLP1(tss1500) |
| cg06861037 | 50278918 | − | N_Shore | |
| cg17765892 | 97156081 | + | N_Shore | GDF6(body) |
| cg06679270 | 1.01E+08 | + | Island | CERS3(tss200) |
| cg00318347 | 25812500 | − | | EBF2(body) |
| cg19433435 | 1.25E+08 | + | S_Shore | FEZ1(tss1500) |
| cg24435747 | 24514557 | − | S_Shore | IFNLR1(tss1500) |
| cg15371026 | 63682393 | − | | |
| cg22258713 | 1.44E+08 | − | Island | |
| cg14192299 | 46417639 | − | Island | NANOS2(body) |
| cg15837308 | 32427751 | + | | |
| cg18049154 | 1.1E+08 | + | N_Shore | TSLP(body); TSLP(tss1500) |
| cg22872778 | 48494156 | − | N_Shore | ABCA13(body) |
| cg23390118 | 2339513 | − | Island | CACNA1C(body) |
| cg17976873 | 23352295 | + | N_Shelf | REM2(tss200) |
| cg03904291 | 1.19E+08 | + | N_Shore | ABCG4(tss1500) |
| cg09179916 | 38445834 | − | | EGFLAM(body) |
| cg12453228 | 41338345 | − | N_Shore | |
| cg25547332 | 1.31E+08 | + | Island | LOC728637(tss200) |
| cg23687098 | 1.32E+08 | + | Island | |
| cg05917111 | 75568999 | + | | CHST5(body) |
| cg21542078 | 49700006 | − | Island | TRPM4(body) |
| cg10697737 | 1673498 | − | S_Shore | |
| cg18646240 | 40626599 | + | N_Shore | RLF(tss1500) |
| cg20364512 | 28199338 | − | S_Shore | |
| cg19734801 | 1.31E+08 | − | Island | LOC728637(body) |
| cg17016026 | 45203363 | + | | |
| cg18050844 | 44909987 | + | | |
| cg09852697 | 3616642 | − | | TP73(body) |
| cg20367266 | 1.03E+08 | − | | TCEAL7(body) |
| cg19234738 | 1.34E+08 | + | N_Shore | |
| cg09183151 | 1.71E+08 | − | | |
| cg21471580 | 22132874 | − | Island | PIWIL2(body); PIWIL2(tss1500) |
| cg25447894 | 41957443 | − | | CSDC2(body) |
| cg05687834 | 1.07E+08 | + | S_Shore | NUAK1(tss1500) |
| cg09544050 | 1.44E+08 | − | Island | BAI1(body) |
| cg18184975 | 3644192 | + | S_Shelf | GFRA4(tss200) |
| cg12806797 | 14926572 | + | N_Shore | H2AFJ(tss1500) |
| cg24969520 | 45359715 | − | N_Shore | TMEM72-AS1(body) |
| cg22457769 | 1.45E+08 | + | S_Shore | FAM83H-AS1(body) |
| cg01025774 | 42944457 | + | Island | |
| cg14281592 | 1.3E+08 | + | | ANGPTL2(body); RALGPS1(body) |
| cg20358011 | 1.27E+08 | + | | KIRREL3(body) |
| cg06174495 | 47399648 | + | N_Shore | |
| cg23651872 | 77924733 | − | S_Shore | TBC1D16(body); TBC1D16(tss200) |
| cg10085138 | 25991722 | + | S_Shore | |
| cg18209323 | 92672878 | + | Island | |
| cg09533178 | 55630336 | + | S_Shore | PPP1R12C(tss1500) |
| cg09408902 | 1.21E+08 | + | S_Shelf | |
| cg24920126 | 5087266 | + | S_Shore | PPP1R3G(body) |
| cg20918682 | 6034694 | − | S_Shore | LRRN4(body) |
| cg03846641 | 1.1E+08 | + | Island | SH3RF3(body); SH3RF3-AS1(tss200) |
| cg06108383 | 32120899 | − | N_Shore | LOC100507547(body); PPT2(tss1500); |
| | | | | PPT2- |
| | | | | EGFL8(tss1500); PRRT1(tss1500) |
| cg05189804 | 1.23E+08 | − | N_Shore | CLMP(body) |
| cg02726377 | 1.09E+08 | − | N_Shore | SGMS2(tss1500) |
| cg12671565 | 1904730 | + | N_Shore | CACNA2D4(body) |
| cg10608596 | 11785125 | − | Island | ZNF833P(body) |
| cg05156901 | 51016646 | − | Island | CHKB- |
| | | | | CPT1B(body); CPT1B(body); CPT1B |
| | | | | (tss200) |
| cg21033965 | 1.78E+08 | − | Island | |
| cg10531073 | 38485757 | + | S_Shore | BAIAP2L2(body) |
| cg10441379 | 72793693 | − | N_Shore | BTF3(tss1500) |
| cg04136369 | 36070600 | − | | HNF1B(body) |
| cg07068045 | 69312202 | + | | |
| cg03221914 | 27783331 | − | S_Shore | HIST1H2AJ(tss1500) |
| cg24829409 | 58192753 | − | Island | LINC00588(body) |
| cg21734175 | 1.21E+08 | + | | |
| cg00056497 | 1.35E+08 | − | | LOC399829(body) |
| cg22158992 | 1021831 | − | N_Shelf | NKD2(body) |
| cg13331196 | 88295591 | − | Island | |
| cg11586382 | 3215966 | − | S_Shore | PITRM1(tss1500) |
| cg27449258 | 46493436 | + | N_Shore | ADARB1(tss1500); SSR4P1(tss1500) |
| cg24576535 | 1.13E+08 | − | S_Shelf | |
| cg14836839 | 42706084 | + | | |
| cg03814644 | 1.66E+08 | − | Island | LINC00473(body) |
| cg07033475 | 13508769 | + | | |
| cg23812775 | 48416899 | + | S_Shelf | GDF2(tss200) |
| cg00147172 | 46423604 | + | Island | |
| cg19176553 | 43049895 | + | S_Shore | |
| cg20444256 | 45826341 | + | N_Shore | CKM(tss200) |
| cg19478539 | 39957639 | + | | |
| cg20366479 | 2.18E+08 | − | S_Shelf | IGFBP2(body) |
| cg10875257 | 28554274 | − | N_Shore | SCAND3(body) |
| cg15013527 | 316339 | − | Island | |
| cg09676390 | 46493345 | − | N_Shore | ADARB1(tss1500); SSR4P1(tss1500) |
| cg21902394 | 19883326 | − | Island | |
| cg02463440 | 22132932 | − | Island | PIWIL2(body); PIWIL2(tss200) |
| cg11170318 | 45246363 | − | Island | |
| cg14837974 | 21110908 | − | | OR6S1(tss1500) |
| cg25845463 | 2064046 | − | Island | PRKCZ(body) |
| cg02738086 | 41941494 | + | S_Shore | POLR3H(tss1500) |
| cg19398496 | 26323437 | + | | |
| cg17872886 | 3542732 | − | Island | COLEC11(body) |
| cg20673321 | 48049233 | − | S_Shore | ZNF541(body) |
| cg07855242 | 1841068 | + | N_Shore | MYT1L(body) |
| cg19570321 | 63258214 | + | N_Shore | HRASLS5(body); MIR3680- |
| | | | | 1(body); MIR3680-2(body) |
| cg21116077 | 10920786 | − | | |
| cg22508928 | 10710134 | − | S_Shore | CASZ1(body) |
| cg17239057 | 2.24E+08 | + | | |
| cg16559243 | 38445612 | − | | EGPLAM(body) |
| cg18055623 | 54485327 | + | Island | CACNG8(body); MIR935(tss1500) |
| cg24569251 | 36628087 | − | N_Shore | |
| cg03961510 | 2978438 | + | | |
| cg00995520 | 1.11E+08 | − | S_Shore | KCNA3(tss1500) |
| cg02486145 | 38334178 | + | | |
| cg00245465 | 8756853 | + | N_Shore | AICDA(body) |
| cg27071797 | 62516631 | + | Island | |
| cg00348467 | 43222121 | − | Island | |
| cg05526015 | 1.71E+08 | + | | |
| cg25594486 | 51165441 | − | Island | SHANK1(body) |
| cg22039909 | 3635100 | − | Island | TP73(body) |
| cg27126225 | 8101793 | + | N_Shore | TUB(body); TUB(tss1500) |
| cg19717326 | 54370016 | + | N_Shore | MYADM(body); MYADM(tss1500) |
| cg22914338 | 14376960 | − | Island | |
| cg03998792 | 1.2E+08 | + | S_Shelf | |
| cg12125332 | 75536561 | + | S_Shore | ZC2HC1C(body) |
| cg09320367 | 40177469 | + | | |
| cg02680487 | 30851529 | + | N_Shore | DDR1(tss1500) |
| cg02477448 | 38199880 | + | N_Shore | H1F0(tss1500) |
| cg05341260 | 37730450 | − | S_Shore | |
| cg14833891 | 45188085 | + | | |
| cg19975916 | 1.23E+08 | + | | HCAR1(tss200) |
| cg06074123 | 56660460 | + | S_Shore | |
| cg15234845 | 1.02E+08 | + | N_Shore | |
| cg04636402 | 1.39E+08 | + | S_Shore | NRG2(body) |
| cg01847021 | 4877595 | − | S_Shore | RADIL(body) |
| cg00850481 | 2.29E+08 | − | S_Shore | |
| cg19999682 | 14864863 | − | S_Shore | |
| cg01467047 | 86611690 | + | N_Shore | FOXL1(tss1500) |
| cg14128068 | 96590527 | + | S_Shore | |
| cg16468843 | 10370624 | − | Island | ATP2B2(body) |
| cg14209784 | 88729861 | + | N_Shore | ADIRF(body); AGAP11(tss1500) |
| cg08383929 | 1.81E+08 | + | Island | |
| cg24942458 | 33723713 | + | N_Shelf | |
| cg24530234 | 44224014 | + | Island | |
| cg11560654 | 27317117 | + | | |
| cg13507429 | 18807550 | − | N_Shore | KLHDC7A(body) |
| cg13796381 | 1.4E+08 | + | Island | PTGDS(body) |
| cg10350722 | 3490214 | − | | PRMT8(tss1500) |
| cg20758542 | 17565149 | + | N_shore | IL17RA(tss1500) |
| cg19443920 | 40075879 | + | S_Shore | ACLY(tss1500) |
| cg21890667 | 30476089 | + | Island | HORMAD2(tss1500) |
| cg10068408 | 62783394 | − | | SLC22A8(tss200) |
| cg08350047 | 50278722 | + | N_Shore | |
| cg16081457 | 81103680 | − | S_Shore | |
| cg23018236 | 30244553 | + | Island | |
| cg09856367 | 1.34E+08 | − | N_Shore | |
| cg11320749 | 1.3E+08 | − | Island | |
| cg05434641 | 56391437 | + | | |
| cg27648238 | 1.21E+08 | + | | |
| cg23657179 | 77165025 | + | Island | ZNF503-AS2(body) |
| cg23367683 | 1.81E+08 | + | N_Shelf | TRIM7(body); TRIM7(tss200) |
| cg03630736 | 74069290 | − | N_Shore | SRP68(tss1500) |
| cg19402371 | 55889013 | + | Island | TMEM190(body) |
| cg21833184 | 5112572 | − | S_Shore | RBAK- |
| | | | | RBAKDN(body); RBAKDN(body) |
| cg05216268 | 42328872 | + | | PLA2G4E(body) |
| cg07168142 | 40274740 | − | Island | HSPB9(tss200); KAT2A(tss1500) |
| cg14625581 | 75692478 | + | | |
| cg10432947 | 2062441 | − | Island | NAT8L(body) |
| cg19710238 | 382237 | − | | |
| cg27001715 | 1.5E+08 | − | S_Shelf | |
| cg22549041 | 75019251 | + | Island | CYP1A1(tss1500) |
| cg26952618 | 10912545 | − | Island | TVP23A(body) |
| cg14785464 | 38362725 | + | Island | HLCS(tss200) |
| cg08819022 | 18531447 | + | Island | |
| cg09065876 | 45949756 | − | Island | |
| cg12440062 | 49681391 | + | | CRISP2(tss200) |
| cg01332054 | 74494861 | + | | STRA6(body) |
| cg00964751 | 36828900 | + | N_Shore | C17orf96(body) |
| cg19112186 | 51016638 | − | Island | CHKB- |
| | | | | CPT1B(body); CPT1B(body); CPT1B |
| | | | | (tss200) |
| cg13583088 | 94360154 | − | | |
| cg11367159 | 1.1E+08 | − | | |
| cg26784300 | 8596908 | − | Island | |
| cg27464846 | 88268600 | − | S_Shore | |
| cg19463349 | 27316791 | + | | |
| cg16689481 | 1.52E+08 | + | N_Shore | TCHH(body) |
| cg26875665 | 1.6E+08 | − | Island | NHLH1(body) |
| cg22251148 | 16631866 | + | N_Shore | |
| cg03983336 | 1.09E+08 | + | N_Shore | SLC5A7(tss1500) |
| cg15302032 | 1.32E+08 | − | | NTM(body) |
| cg02207638 | 33880049 | + | Island | FAM83C(body) |
| cg11868728 | 22568116 | − | S_Shore | |
| cg00989365 | 70014548 | + | Island | |
| cg21477262 | 22777430 | − | N_Shore | ETNK1(tss1500) |
| cg27569858 | 1.11E+08 | + | S_Shore | ALX3(tss1500) |
| cg01239922 | 191242 | + | N_Shore | LRRC14B(tss1500) |
| cg00947878 | 57832005 | − | N_Shelf | KIFC3(body); KIFC3(tss200) |
| cg24268343 | 56260186 | − | S_Shore | KLF8(body) |
| cg20925954 | 1.12E+08 | + | N_Shore | DUSP5(tss1500) |
| cg27314669 | 1.57E+08 | − | S_Shelf | INSRR(body); NTRK1(body) |
| cg08222513 | 30079280 | − | | TRIM31(body) |
| cg11632438 | 1.59E+08 | − | N_Shore | MFSD1(tss1500) |
| cg17369032 | 47590326 | + | Island | LOC100288866(body); NGFR(body) |
| cg19983801 | 1.96E+08 | + | | MUC4(body) |
| cg14843888 | 53530247 | − | S_Shore | CACNA1D(body) |
| cg04502620 | 30020843 | − | N_Shore | DOC2A(body) |
| cg27297505 | 1.21E+08 | − | | |
| cg01466011 | 1.57E+08 | − | N_Shore | LINC00880(body) |
| cg11498156 | 1.03E+08 | + | N_Shore | TLX1(tss1500); TLX1NB(body) |
| cg07923794 | 77834237 | − | Island | |
| cg19362196 | 26233615 | + | Island | MIR3917(tss1500); STMN1(tss1500) |
| cg19045731 | 2.39E+08 | − | Island | ESPNL(body) |
| cg10225865 | 54605566 | − | | |
| cg15541170 | 2.38E+08 | + | | |
| cg16426293 | 40192112 | + | Island | |
| cg03243506 | 2517231 | + | | TMC2(tss200) |
| cg08639948 | 36591237 | − | S_Shore | |
| cg04534697 | 1.64E+08 | + | Island | |
| cg19313311 | 74058031 | + | | |
| cg16355231 | 2344979 | − | N_Shore | PEX10(tss1500) |
| cg11998619 | 48364243 | + | S_Shore | |
| cg04164023 | 1.25E+08 | + | | PKNOX2(body) |
| cg09713684 | 1.34E+08 | − | N_Shore | C5orf66(body) |
| cg13206556 | 20875399 | + | N_Shore | |
| cg04647918 | 67383862 | − | S_Shore | DOC2GP(tss1500) |
| cg19894382 | 60086891 | − | | |
| cg16617366 | 46064816 | − | N_Shore | CTIF(tss1500) |
| cg25270844 | 56004925 | − | N_Shore | SSC5D(body) |
| cg16537676 | 30851624 | − | N_Shore | DDR1(tss1500) |
| cg14951955 | 1.07E+08 | − | S_Shore | ATG5(tss1500) |
| cg15986919 | 55887799 | + | N_Shore | TMEM190(tss1500) |
| cg00305229 | 46943714 | + | S_Shelf | |
| cg03929570 | 70484978 | − | N_Shelf | |
| cg02788857 | 22132959 | − | Island | PIWIL2(body); PIWIL2(tss200) |
| cg01362541 | 55838142 | + | N_Shore | BMP7(body) |
| cg24435209 | 17640972 | + | S_Shore | CECR5(body); CECR5(tss1500); CECR5- |
| | | | | AS1(body) |
| cg16748643 | 1336696 | + | | |
| cg13561807 | 44237619 | − | S_Shore | LOXHD1(tss1500) |
| cg24258529 | 1570685 | + | Island | |
| cg22695379 | 1.38E+08 | − | Island | GFRA3(body) |
| cg01677628 | 8361190 | + | | |
| cg04487205 | 36997563 | + | Island | C17orf98(body) |
| cg04915618 | 1.24E+08 | − | S_Shore | KALRN(body) |
| cg07790079 | 1.16E+08 | + | | |
| cg21911276 | 55289797 | + | S_Shore | NARS(tss1500) |
| cg23820816 | 1.54E+08 | − | | TRIM2(body); TRIM2(tss200) |
| cg26036375 | 1.23E+08 | + | | |
| cg02661079 | 44829722 | − | Island | CDH22(body) |
| cg20418125 | 1.33E+08 | + | | OPCML(body) |
| cg23146346 | 17282859 | + | S_Shore | RBM24(body); RBM24(tss200) |
| cg26590664 | 11784731 | − | N_Shore | ZNF833P(tss200) |
| cg19431051 | 60414767 | + | Island | LINC00301(body) |
| cg02283735 | 44011587 | + | | PTPRF(body) |
| cg17158564 | 5406890 | + | | UCN3(tss200) |
| cg05936300 | 3574704 | − | | TP73(body) |
| cg25288155 | 73357396 | + | | PLEKHB1(body); PLEKHB1(tss1500) |
| cg23079252 | 30095199 | − | Island | |
| cg27583138 | 24774877 | − | N_Shore | GMNN(tss1500) |
| cg05393297 | 53359155 | − | N_Shore | |
| cg03710889 | 2.19E+08 | − | S_Shore | SLC11A1(body) |
| cg16080552 | 59043199 | + | Island | TACSTD2(tss200) |
| cg04217177 | 2.17E+08 | + | N_Shore | ESRRG(body) |
| cg06620514 | 74793271 | + | | |
| cg17878151 | 99975334 | + | | |
| cg00892368 | 32847704 | − | Island | |
| cg23863328 | 49879704 | − | | |
| cg22805485 | 13983818 | − | N_Shore | SPON1(tss1500) |
| cg26976412 | 17626127 | − | Island | RAI1(body) |
| cg00646396 | 26482755 | − | | |
| cg04888360 | 99648780 | − | | CRTAC1(body) |
| cg15901783 | 77461426 | − | S_Shore | KCTD12(tss1500) |
| cg02354563 | 522363 | + | N_Shore | SLC9A3(body) |
| cg16092017 | 1.35E+08 | + | Island | |
| cg00317879 | 68920611 | + | | LOC338694(body) |
| cg02557933 | 2.2E+08 | + | S_Shore | SPEG(body) |
| cg26206185 | 50451151 | + | N_Shore | IL17REL(tss200) |
| cg12583970 | 27190330 | + | N_Shore | SFN(body) |
| cg03954393 | 2.05E+08 | − | | |
| cg05756918 | 27253707 | − | Island | |
| cg09035529 | 64335804 | − | Island | SLC22A11(body) |
| cg27291468 | 9878816 | + | | |
| cg02369725 | 1.79E+08 | + | N_Shore | PDE11A(body) |
| cg19871388 | 50419082 | + | S_Shore | SALL4(tss200) |
| cg27516568 | 1.23E+08 | − | S_Shore | PP12613(body); TMEM155(tss1500) |
| cg11537406 | 1.25E+08 | − | N_Shore | SLC37A2(tss1500) |
| cg12231373 | 36628103 | − | N_Shore | |
| cg15743907 | 1.45E+08 | − | | LOC100288142(body); NBPF9(body); |
| | | | | PDE4DIP(body) |
| cg15193133 | 19883474 | + | Island | |
| cg03727673 | 21966420 | + | | RAP1GAP(body) |
| cg10816626 | 10711457 | + | N_Shelf | CASZ1(body) |
| cg18135555 | 22132992 | − | Island | PIWIL2(body); PIWIL2(tss200) |
| cg19370715 | 10710847 | − | N_Shelf | CASZ1(body) |
| cg06501366 | 78365687 | + | Island | BHMT2(body); DMGDH(tss200) |
| cg09071762 | 74064149 | + | Island | DPPA5(tss200) |
| cg01306688 | 1.23E+08 | + | | HCAR1(tss200) |
| cg18322589 | 1.24E+08 | + | | TACC2(body) |
| cg26090072 | 74669387 | + | Island | RTKN(tss1500) |
| cg04876424 | 17280551 | + | N_Shore | RBM24(tss1500) |
| cg04605287 | 54953486 | + | N_Shore | |
| cg08109624 | 2.42E+08 | + | Island | |
| cg11168104 | 1857477 | − | Island | |
| cg14030674 | 41654557 | − | N_Shore | ANK1(body) |
| cg05168015 | 1.72E+08 | + | N_Shore | SH3PXD2B(body) |
| cg00256046 | 2.17E+08 | − | N_Shore | ESRRG(body) |
| cg12102973 | 13320368 | − | S_Shore | CACNA1A(body) |
| cg02932167 | 2.33E+08 | − | Island | ECEL1(tss1500) |
| cg08234653 | 44223817 | − | Island | |
| cg10347828 | 49244040 | − | S_Shore | RASIP1(tss200) |
| cg18942934 | 32847762 | + | Island | |
| cg02599361 | 1.79E+08 | + | Island | ADAMTS2(body) |
| cg01854076 | 2063036 | − | Island | NAT8L(body) |
| cg05021771 | 71129289 | − | N_Shore | VAX2(body) |
| cg14882481 | 1.07E+08 | − | | ALKBH8(tss1500) |
| cg17830682 | 2.02E+08 | + | N_Shore | LGR6(body); LGR6(tss1500) |
| cg07241675 | 99067143 | + | N_Shelf | |
| cg08889009 | 59157123 | + | Island | |
| cg11218842 | 5406907 | + | | UCN3(tss200) |
| cg02168442 | 1.02E+08 | + | Island | |
| cg19766489 | 78511344 | − | N_Shore | GIPC2(tss1500) |
| cg08217910 | 1.2E+08 | − | N_Shore | TMEM37(tss1500) |
| cg24720008 | 44788044 | − | N_Shore | LOC100130539(tss200) |
| cg02657828 | 86703935 | − | | |
| cg03446244 | 86229872 | − | | LINC01082(body) |
| cg18026225 | 43198423 | − | Island | PLCD3(body) |
| cg01960748 | 24522592 | − | | |
| cg21136104 | 1.48E+08 | + | Island | |
| cg27522367 | 50497732 | + | | GPD1(body) |
| cg13344095 | 26233352 | + | | |
| cg23206461 | 1.48E+08 | + | Island | NBPF8(body) |
| cg11704513 | 30225851 | + | Island | COX4I2(body) |
| cg27469738 | 99338074 | − | Island | ANKRD2(body) |
| cg22243109 | 75789365 | − | Island | |
| cg18869061 | 1E+08 | + | N_Shore | LYSMD4(body) |
| cg13490352 | 55072042 | − | N_Shore | |
| cg12588880 | 8597159 | − | Island | |
| cg05820241 | 2.32E+08 | + | | |
| cg04037236 | 2951736 | + | N_Shore | |
| cg01601573 | 1.44E+08 | − | N_Shore | GAB1(tss1500) |
| cg24762889 | 29127871 | − | Island | |
| cg13244241 | 1.02E+08 | − | S_Shore | |
| cg13291570 | 514736 | + | | |
| cg04473902 | 14721288 | + | S_Shore | PLBD1(tss1500) |
| cg05567511 | 1.51E+08 | − | | |
| cg05950877 | 11784647 | − | N_Shore | ZNF833P(tss200) |
| cg05239811 | 31734147 | − | | VWA7(body) |
| cg06251500 | 1.51E+08 | − | N_Shore | KCNH2(body) |
| cg09517873 | 12656315 | + | S_Shore | DHRS3(body) |
| cg02735058 | 16181250 | − | | |
| cg16288318 | 1.13E+08 | − | S_Shore | |
| cg20518446 | 62315034 | + | Island | AHNAK(tss1500) |
| cg13001017 | 74072320 | + | N_Shore | KHDC3L(tss200) |
| cg20236750 | 95516908 | − | | LGI1(tss1500) |
| cg08217285 | 27917879 | + | N_Shore | GIT1(tss1500) |
| cg24723561 | 2.34E+08 | − | S_Shore | SLC35F3(body) |
| cg08250742 | 47651453 | + | N_Shore | LOC100288866(tss200) |
| cg11556846 | 2E+08 | − | Island | |
| cg03130418 | 32063439 | + | N_Shore | TNXB(body) |
| cg08950537 | 95361933 | + | S_Shore | RBP4(tss1500) |
| cg13464393 | 90440066 | + | Island | |
| cg14170358 | 88527345 | + | | |
| cg15597109 | 10711695 | − | N_Shelf | CASZ1(body) |
| cg05358404 | 62288950 | + | Island | RTEL1(tss1500); RTEL1- |
| | | | | TNFRSF6B(tss1500) |
| cg04143909 | 2.34E+08 | − | Island | SLC35F3(body) |
| cg05411132 | 99195115 | − | S_Shore | XRCC6P5(tss1500) |
| cg21489266 | 46619131 | − | S_Shore | TDGF1(body) |
| cg22572476 | 28601324 | − | N_Shore | |
| cg20071884 | 1.18E+08 | + | Island | KIAA1210(tss200) |
| cg19910200 | 55888150 | − | N_Shore | TMEM190(tss200) |
| cg01869233 | 6035139 | + | S_Shore | LRRN4(tss1500) |
| cg14283328 | 66823587 | − | N_Shore | RHOD(tss1500) |
| cg18533225 | 50986813 | − | Island | KLHDC7B(body) |
| cg07654896 | 46937829 | + | Island | RGN(body) |
| cg03021802 | 21923841 | − | Island | DMTN(body) |
| cg21391046 | 1.84E+08 | + | | |
| cg21251018 | 28226885 | + | N_Shore | NKAPL(tss200) |
| cg09906233 | 2341263 | − | Island | ZFYVE28(body) |
| cg07476339 | 1.55E+08 | − | S_Shore | |
| cg08952407 | 42756860 | − | | CCDC152(tss200) |
| cg23936402 | 28499703 | − | | GPX5(body) |
| cg02172058 | 58499911 | − | S_Shore | C17orf64(body) |
| cg08812802 | 49686619 | − | N_Shelf | |
| cg20432211 | 77342104 | + | Island | |
| cg04731038 | 48858636 | − | Island | |
| cg21716282 | 1.28E+08 | + | Island | |
| cg15769343 | 1.21E+08 | + | Island | |
| cg07207789 | 84852909 | − | N_Shore | CRISPLD2(tss1500) |
| cg24263062 | 2730191 | + | Island | EBF4(body) |
| cg13029400 | 1.41E+08 | − | | ZBTB38(body) |
| cg02834449 | 85786110 | − | S_Shore | C16orf74(tss1500) |
| cg03124514 | 42943857 | − | N_Shore | |
| cg17519696 | 28148266 | − | Island | OCA2(body) |
| cg25985355 | 65971099 | − | Island | |
| cg10043155 | 25218855 | + | | |
| cg16595288 | 43698931 | + | S_Shore | RASGEF1A(body) |
| cg00737841 | 1.31E+08 | + | | |
| cg26687119 | 1.57E+08 | − | N_Shore | INSRR(body); NTRK1(body) |
| cg15638292 | 76849451 | − | Island | MYO7A(body) |
| cg27196974 | 80337729 | + | | |
| cg01306824 | 77834118 | − | Island | |
| cg23105130 | 1.45E+08 | − | Island | MAPK15(body) |
| cg07726287 | 84318109 | + | | |
| cg14759277 | 48674158 | + | | CACNA1G(body) |
| cg05134926 | 30349111 | + | N_Shore | KLHL14(body) |
| cg12161935 | 214148 | − | Island | |
| cg08134856 | 26233709 | + | S_Shore | MIR3917(tss1500); STMN1(tss1500) |
| cg17870997 | 1.21E+08 | + | N_Shore | |
| cg04169774 | 2.39E+08 | − | S_Shore | PER2(tss1500) |
| cg13404038 | 1.26E+08 | + | | CRB2(body) |
| cg27357306 | 63657059 | + | | |
| cg21434046 | 56435611 | + | N_Shore | |
| cg01161644 | 28270238 | − | N_Shore | |
| cg12432038 | 2.47E+08 | − | Island | C1orf229(tss200) |
| cg19628964 | 28830011 | + | N_Shore | LOC401242(body) |
| cg02462416 | 2036573 | − | S_Shelf | |
| cg03696370 | 22642451 | + | | PEBP4(body) |
| cg02784823 | 49000897 | + | Island | LMTK3(body) |
| cg21936959 | 68782049 | + | S_Shelf | MRGPRF(tss1500) |
| cg15770575 | 42760647 | + | N_Shelf | |
| cg07991586 | 50279175 | − | N_Shore | |
| cg07455790 | 31650735 | − | Island | |
| cg03671431 | 61604367 | + | S_Shore | |
| cg21151061 | 50498016 | − | | GPD1(body) |
| cg14476334 | 1100557 | − | Island | |
| cg27237745 | 74072271 | − | N_Shore | KHDC3L(tss200) |
| cg09101902 | 49863809 | + | Island | ARHGAP22(body) |
| cg21597025 | 1.43E+08 | − | N_Shore | CHST2(tss1500) |
| cg07589381 | 10764512 | + | Island | CASZ1(body) |
| cg11823142 | 27018041 | − | | |
| cg12130768 | 9550545 | + | S_Shore | USP43(body) |
| cg27206976 | 50738315 | − | Island | PLXNB2(body) |
| cg22496723 | 36960779 | + | Island | CACNG2(body) |
| cg20031364 | 36731152 | − | S_Shore | SRCIN1(body) |
| cg14457311 | 48698799 | − | | CACNA1G(body) |
| cg13211302 | 1.02E+08 | − | Island | |
| cg11348165 | 27189270 | − | N_Shore | SFNU(tss1500) |
| cg19545472 | 3644097 | + | S_Shore | GFRA4(tss200) |
| cg05056638 | 24800824 | − | S_Shore | |
| cg12207153 | 38519255 | + | | |
| cg16351246 | 20751345 | − | Island | |
| cg13053129 | 1311811 | + | N_Shore | |
| cg27503360 | 43890749 | − | | CRHR1(body) |
| cg09552892 | 88718274 | + | | MMRN2(tss1500); SNCG(tss200) |
| cg24049888 | 1.11E+08 | + | | POU2AF1(body) |
| cg21461564 | 63999030 | − | S_Shore | TTPA(tss1500) |
| cg03661817 | 57831745 | − | | KIFC3(body) |
| cg00847857 | 30565636 | + | | LINC00189(tss200) |
| cg02141929 | 56681544 | + | S_Shelf | |
| cg09817162 | 27185676 | + | | |
| cg26182859 | 1.54E+08 | − | | |
| cg27179866 | 27740120 | − | | |
| cg25341560 | 35530755 | + | N_Shore | HPN(tss1500); SCN1B(body) |
| cg09765983 | 1.54E+05 | − | | |
| cg00274965 | 34405681 | + | Island | |
| cg03115048 | 1.52E+08 | − | N_Shelf | |
| cg15534366 | 59826207 | − | N_Shore | CDH4(tss1500) |
| cg13125537 | 7037323 | + | | |
| cg06314987 | 86611714 | − | N_Shore | FOXL1(tss1500) |
| cg25604594 | 99338188 | − | Island | ANKRD2(body) |
| cg26477792 | 30095277 | + | Island | |
| cg23467079 | 1.24E+08 | + | Island | FAM83A(body) |
| cg00592643 | 11870769 | − | N_Shore | BTBD3(tss1500) |
| cg01862363 | 8928028 | − | S_Shore | NTN1(body) |
| cg03879180 | 24713283 | − | N_Shore | |
| cg13744039 | 1.84E+08 | − | | |
| cg24261333 | 73357019 | + | | PLEKHB1(tss1500) |
| cg23335299 | 90291756 | + | N_Shore | |
| cg18376352 | 18706136 | + | Island | CRLF1(body) |
| cg01914059 | 88448561 | + | N_Shore | |
| cg10958002 | 12200324 | + | Island | SYN2(body); TIMP4(body) |
| cg14785609 | 1.58E+08 | + | Island | |
| cg17962638 | 90597340 | − | Island | |
| cg26535593 | 2.47E+08 | + | S_Shore | ZNF670(tss1500); ZNF670- |
| | | | | ZNF695(tss1500) |
| cg08745665 | 10460009 | − | S_Shore | ZNF518B(tss1500) |
| cg09506575 | 1.13E+08 | + | | GPR85(tss1500); GPR85(tss200) |
| cg20941258 | 46618668 | + | Island | TDGF1(body); TDGF1(tss1500) |
| cg06891815 | 32165237 | + | S_Shore | NOTCH4(body) |
| cg21062347 | 62783543 | − | | SLC22A8(tss1500) |
| cg08260245 | 51016501 | + | Island | CHKB-CPT1B(body); CPT1B(body) |
| cg21700780 | 2004050 | − | Island | MYOM2(body) |
| cg25792518 | 48545950 | − | Island | ACSF2(body); CHAD(body) |
| cg08169827 | 32054766 | − | Island | |
| cg11715966 | 46917255 | + | S_Shore | CCDC8(tss1500) |
| cg01733570 | 24797656 | + | S_Shore | DFNA5(tss1500); DFNA5(tss200) |
| cg12758973 | 2.48E+08 | − | Island | GCSAML(body); GCSAML- |
| | | | | AS1(tss200); OR2C3(body) |
| cg21051964 | 58022620 | − | S_Shore | B4GALNT1(body) |
| cg12499235 | 2293173 | − | S_Shore | ASCL2(tss1500) |
| cg04149978 | 10764896 | + | Island | CASZ1(body) |
| cg09795979 | 45308098 | + | S_Shore | SYT13(tss1500) |
| cg06260709 | 48626621 | − | Island | |
| cg03456771 | 56242072 | − | N_Shore | |
| cg18062205 | 49630819 | − | S_Shelf | KCNG1(body) |
| cg00095526 | 2.01E+08 | + | N_Shore | CACNA1S(body) |
| cg00944166 | 1.02E+08 | − | Island | SLCO6A1(body) |
| cg19872936 | 76932055 | − | | ESSRB(body) |
| cg14320626 | 3746025 | − | N_Shelf | |
| cg26014401 | 25082111 | − | Island | |
| cg23823894 | 11471881 | − | S_Shore | |
| cg14724749 | 51774592 | − | Island | |
| cg03547924 | 34025927 | + | S_Shelf | GDF5(body) |
| cg27147718 | 58496542 | + | N_Shore | NDRG4(tss1500) |
| cg00108349 | 24914657 | − | Island | |
| cg27454064 | 64215611 | + | Island | |
| cg14892768 | 41725332 | − | | AXL(body) |
| cg11213150 | 1.3E+08 | + | | ANGPTL2(body); RALGPS1(body) |
| cg20951444 | 33337112 | − | | FNDC5(body); FNDC5(tss1500) |
| cg12602851 | 76644222 | − | | IMPG1(body) |
| cg04079760 | 68269694 | − | Island | ESRP2(body) |
| cg10036920 | 1.33E+08 | − | Island | LRCOL1(body) |
| cg26608305 | 925416 | + | Island | AP2A2(tss1500) |
| cg26516532 | 17475832 | + | Island | |
| cg13872005 | 1.05E+05 | − | Island | |
| cg02049405 | 30095265 | − | Island | |
| cg03622666 | 49499091 | − | Island | |
| cg21950166 | 27189349 | − | N_Shore | SFN(tss1500) |
| cg19250304 | 3215771 | − | S_Shore | PITRM1(tss1500) |
| cg09163477 | 1.31E+08 | + | S_Shore | |
| cg18706028 | 6292490 | − | Island | CCKBR(body) |
| cg19340941 | 1.56E+08 | + | Island | RBM46(body) |
| cg17745392 | 51050258 | − | N_Shore | LRRC4B(body) |
| cg18092474 | 75019302 | + | Island | CYP1A1(tss1500) |
| cg07740525 | 1.17E+08 | − | Island | KIF12(tss200) |
| cg08922729 | 21913557 | − | | |
| cg11885357 | 1.38E+08 | − | N_Shore | ESYT3(tss1500) |
| cg24466377 | 46268509 | + | N_Shore | SIX5(body) |
| cg17105139 | 74729279 | + | N_Shore | LBX2(body); LBX2-AS1(tss1500) |
| cg17842821 | 1834682 | + | | |
| cg27379787 | 2.4E+08 | − | | |
| cg00647658 | 54227814 | − | N_Shore | |
| cg19966745 | 1.35E+08 | − | | |
| cg26233253 | 31032613 | + | S_Shore | SLC35E4(body) |
| cg24992471 | 47571963 | + | N_Shore | NGFR(tss1500) |
| cg23602058 | 48154061 | − | | ITGA3(body) |
| cg06766016 | 45278971 | − | Island | BTBD19(body) |
| cg08012287 | 66315298 | − | S_Shore | ACTN3(body) |
| cg14160968 | 77723057 | + | N_Shore | |
| cg00748494 | 23412343 | − | Island | |
| cg08537783 | 10795394 | + | | CASZ1(body) |
| cg04752871 | 1.21E+08 | − | S_Shore | |
| cg01309213 | 331821 | − | N_Shore | ARHGDIG(body); PDIA2(tss1500) |
| cg00868766 | 40364524 | − | | |
| cg21767759 | 48154356 | − | | ITGA3(body) |
| cg19414967 | 49468296 | + | N_Shore | |
| cg00364956 | 33777219 | − | S_Shore | SLFN13(tss1500) |
| cg17572255 | 50676705 | − | | LIMA1(body) |
| cg20048521 | 74072242 | − | N_Shore | KHDC3L(tss200) |
| cg01343041 | 24397787 | − | Island | FAM228A(tss200) |
| cg11460035 | 94245224 | + | N_Shore | LOC643037(tss1500) |
| cg22451657 | 6558088 | + | S_Shore | PLEKHG5(body); PLEKHG5(tss1500) |
| cg25125298 | 71853215 | + | | |
| cg00463502 | 48912548 | + | | WFIKKN2(tss200) |
| cg22462726 | 1.84E+08 | + | Island | |
| cg25926515 | 70321889 | − | S_Shore | TET1(body) |
| cg03127558 | 49223278 | − | Island | MAMSTR(tss1500) |
| cg15042332 | 1.23E+08 | − | | FABP7(body) |
| cg06710596 | 36024279 | − | Island | GAPDHS(tss200) |
| cg21797718 | 34024416 | − | Island | GRM4(body) |
| cg05857999 | 31650760 | − | Island | |
| cg03961824 | 82530967 | + | | |
| cg02213822 | 1.29E+08 | − | Island | |
| cg09259081 | 84538889 | + | Island | TLDC1(tss1500) |
| cg11181985 | 90439888 | − | Island | |
| cg27491557 | 3165885 | + | S_Shelf | |
| cg13641043 | 43198610 | + | Island | PLCD3(body) |
| cg03058346 | 91275170 | − | | |
| cg27483455 | 50475360 | + | Island | ASIC1(body) |
| cg17204557 | 1.21E+08 | − | | LINC01101(body) |
| cg14434213 | 38508780 | + | S_Shore | |
| cg05376267 | 2.26E+08 | − | Island | LEFTY(body) |
| cg02625638 | 1.24E+08 | − | N_Shore | FAM83A(body) |
| cg16749456 | 49699879 | − | Island | TRPM4(body) |
| cg17768691 | 14720948 | + | Island | PLBD1(tss200) |
| cg15667844 | 1.12E+08 | + | N_Shore | DUSP5(tss1500) |
| cg22522920 | 50276512 | − | | FAIM2(body) |
| cg01001533 | 44184868 | + | N_Shore | |
| cg04686778 | 64375218 | − | Island | NRXN2(body) |
| cg25541708 | 1.8E+08 | − | S_Shore | |
| cg00034101 | 40098595 | − | | HEYL(body) |
| cg10838157 | 27819662 | − | | |
| cg16971128 | 37499859 | + | | TMPRSS6(tss200) |
| cg22573675 | 38762739 | + | N_Shore | |
| cg11386011 | 32121156 | + | N_Shore | LOC100507547(body); PPT2(tss1500); |
| | | | | PPT2(tss200); PPT2- |
| | | | | EGFL8(tss1500); PRRT1(tss1500) |
| cg26500033 | 1.67E+08 | − | S_Shelf | |
| cg25308803 | 1.1E+08 | + | Island | SH3RF3(body); SH3RF3-AS1(tss200) |
| cg15891218 | 61748354 | − | S_Shore | TMEM308(body) |
| cg11362935 | 1.11E+08 | − | | POU2AF1(tss200) |
| cg23009355 | 68451396 | + | Island | GAL(tss1500) |
| cg14099457 | 49170794 | − | | LAMB2(tss200) |
| cg04585822 | 72916509 | + | Island | USH1G(body) |
| cg03014829 | 55516872 | − | Island | |
| cg13707623 | 57766574 | + | Island | ZNF831(body) |
| cg00984540 | 1.56E+08 | + | Island | |
| cg13214295 | 75789279 | + | N_Shore | |
| cg17324149 | 1.28E+08 | + | | |
| cg19833103 | 47126551 | − | N_Shore | LRCH1(tss1500) |
| cg26672426 | 1.33E+08 | + | | PTGES(body) |
| cg11166453 | 2.48E+08 | + | Island | GCSAML(body) |
| cg07951355 | 40123717 | + | | |
| cg01382860 | 49700021 | + | Island | TRPM4(body) |
| cg04327247 | 1.01E+08 | − | N_Shore | |
| cg25478132 | 1.03E+08 | + | | TLX1NB(body) |
| cg01440864 | 44892087 | + | N_Shelf | WNT3(body) |
| cg04986934 | 39019908 | + | Island | KST12(body) |
| cg13067553 | 1.48E−08 | + | Island | NBPF8(body) |
| cg19552640 | 1.27E+08 | − | S_Shore | MEGF10(body) |
| cg05308656 | 2.35E+08 | + | Island | ARL4C(tss1500) |
| cg26772540 | 11784672 | + | N_Shore | ZNF833P(tss200) |
| cg18770763 | 724525 | + | N_Shore | EPS8L2(body) |
| cg09119043 | 2.34E+08 | − | Island | SLC35F3(body) |
| cg10257870 | 78985484 | − | | CMYA5(tss200) |
| cg23882131 | 68779826 | + | S_Shore | MRGPRF(body) |
| cg15587771 | 4050875 | − | | SDK1(body) |
| cg24210717 | 50497827 | + | | GPD1(body) |
| cg13948430 | 1.32E+08 | + | Island | |
| cg02150077 | 87098542 | + | | |
| cg06679334 | 35241844 | − | | |
| cg00697301 | 19936477 | + | | |
| cg24258347 | 13983851 | − | N_Shore | SPON1(tss1500) |
| cg16768191 | 1.53E+08 | − | S_Shore | |
| cg21714557 | 1.14E+08 | + | S_Shelf | F10(tss1500) |
| cg14132536 | 1.31E+08 | + | S_Shore | DPM2(body) |
| cg20339859 | 1.34E+08 | + | | |
| cg06012872 | 45374841 | − | | TMEM72-AS1(body) |
| cg24517467 | 1.55E+08 | − | Island | |
| cg17401780 | 1.3E+08 | − | Island | |
| cg16390049 | 32165089 | + | S_Shore | NOTCH4(body) |
| cg19497444 | 2930794 | + | Island | SLC22A18(body) |
| cg13695585 | 30853014 | − | S_Shore | DDR1(body) |
| cg15682101 | 1.69E+08 | − | S_Shelf | |
| cg14896134 | 88295386 | − | Island | |
| cg13666323 | 8751190 | − | N_Shore | |
| cg21644489 | 63312170 | + | | |
| cg02760962 | 35683421 | − | N_Shore | TRIM44(tss1500) |
| cg07356483 | 99791663 | + | S_Shore | CRTAC1(tss1500) |
| cg12288076 | 45170254 | − | Island | OCSTAMP(body) |
| cg25606780 | 47397852 | − | Island | |
| cg02206373 | 89841767 | + | S_Shore | STEAP2(body); STEAP2(tss1500) |
| cg11434671 | 74524611 | − | | CYGB(body); PRCD(body) |
| cg09068286 | 1.8E+08 | − | Island | GFPT2(body) |
| cg16290689 | 31506685 | − | | TEX26(tss200); TEX26-AS1(body) |
| cg03441844 | 1.61E+08 | + | Island | |
| cg21225804 | 1.33E+08 | − | Island | NCS1(tss1500) |
| cg11822175 | 1.03E+08 | + | | |
| cg15203566 | 1.16E+08 | + | Island | |
| cg08272268 | 2E+08 | − | S_Shore | ZNF281(tss1500) |
| cg17726692 | 74240460 | − | | LINC00908(tss200) |
| cg15043318 | 1.71E+08 | + | | |
| cg19143959 | 40572794 | − | N_Shore | |
| cg24341311 | 1.27E+08 | + | | C3orf56(tss200) |
| cg27331665 | 316456 | + | Island | |
| cg07330481 | 37322330 | + | S_Shore | ARL5C(body) |
| cg24694702 | 1.2E+08 | − | | HSPB8(body) |
| cg02365397 | 40573976 | − | Island | ANKRD63(body) |
| cg08035479 | 58172643 | + | Island | LOC286177(tss1500) |
| cg01168289 | 32421514 | − | | WT1(body) |
| cg12451836 | 1.57E+08 | − | S_Shore | |
| cg03832522 | 2.19E+08 | + | Island | RUFY4(tss1500) |
| cg12199221 | 83017078 | + | N_Shore | HAPLN1(tss200) |
| cg13445177 | 1.52E−08 | − | S_Shore | S100A10(tss1500) |
| cg11403027 | 42971411 | − | | |
| cg03340760 | 1.21E+08 | + | | |
| cg09481121 | 78985425 | − | | CMYA5(tss1500) |
| cg14847243 | 1.84E+08 | + | Island | CHRD(body) |
| cg08481464 | 77871618 | − | N_Shore | C10orf11(body) |
| cg14897238 | 43198283 | − | N_Shore | |
| cg14972155 | 54281198 | + | | ESM1(body) |
| cg22624278 | 1.31E+08 | − | | |
| cg07164191 | 1.77E+08 | − | S_Shore | |
| cg18556455 | 45178474 | − | N_Shore | |
| cg03969301 | 11056425 | + | S_Shelf | |
| cg24067118 | 1.03E+08 | + | N_Shore | |
| cg23647410 | 2.21E+08 | − | N_Shore | |
| cg03484180 | 49967517 | + | S_Shore | MON1A(tss200) |
| cg25966908 | 55516751 | − | N_Shore | |
| cg17469265 | 39183353 | − | N_Shore | WDR19(tss1500) |
| cg06468108 | 1.74E+08 | − | | |
| cg01971227 | 3988694 | − | N_Shore | |
| cg10487067 | 40112140 | − | | |
| cg17745234 | 37499649 | + | | TMPRSS6(body) |
| cg18460422 | 32063553 | + | Island | TNXB(body) |
| cg02024846 | 99382466 | + | S_Shore | CDC14B(tss1500) |
| cg25394203 | 11784708 | − | N_Shore | ZNF833P(tss200) |
| cg19149031 | 1.61E+08 | − | Island | SLC22A3(body) |
| cg22471112 | 1.54E+08 | + | | TRIM2(body); TRIM2(tss200) |
| cg23626733 | 2345398 | + | N_Shore | PEX10(tss1500) |
| cg13337731 | 73011308 | − | | MIXIPL(body) |
| cg22445712 | 50738550 | + | Island | PLXNB2(body) |
| cg27385590 | 49619841 | + | | |
| cg05499178 | 69285980 | + | Island | |
| cg03697173 | 4877433 | − | S_Shore | RADIL(body) |
| cg11676353 | 39548001 | − | S_Shore | |
| cg01811796 | 26233565 | + | Island | MIR3917(tss1500); STMN1(tss1500); |
| | | | | STMN1(tss200) |
| cg10217202 | 62331131 | − | Island | |
| cg15302379 | 1.03E+08 | + | Island | KAZALD1(body) |
| cg01499518 | 75890022 | + | S_Shore | SRRM3(body) |
| cg12079699 | 1.32E+08 | − | S_Shelf | NTM(body) |
| cg24414344 | 39019556 | − | Island | KRT12(body) |
| cg00463859 | 23750101 | − | N_Shore | TCEA3(body) |
| cg17213154 | 8832956 | − | | ST5(body); ST5(tss1500); ST5(tss200) |
| cg22523072 | 2.48E+08 | − | | |
| cg08231697 | 1.57E+08 | + | S_Shore | |
| cg02431260 | 1.25E+08 | + | Island | ESAM(body) |
| cg03939719 | 49379062 | + | S_Shelf | |
| cg24599107 | 1.61E+08 | + | Island | PVRL4(body) |
| cg13220917 | 2.42E+08 | − | N_Shore | |
| cg07935320 | 1.24E+08 | + | N_Shore | FAM83A(body) |
| cg24279744 | 10408402 | + | | ATP2B2(body) |
| cg12271415 | 56005437 | + | S_Shore | SSC5D(body) |
| cg03517083 | 75569527 | − | | CHST5(tss1500) |
| cg12793610 | 1.54E+08 | − | | TRIM2(body) |
| cg22550309 | 46875360 | + | Island | PRSS42(body) |
| cg19773546 | 45944339 | − | | |
| cg18045195 | 45178950 | + | N_Shore | |
| cg20800117 | 7308366 | − | | CAMTA1(body) |
| cg26931208 | 49244592 | − | S_Shore | IZUMO1(body); RASIP1(tss1500) |
| cg09411356 | 2.28E+08 | − | N_Shelf | C1orf145(body); OBSCN(tss200) |
| cg06351981 | 2.2E+08 | − | S_Shore | SPEG(body) |
| cg16686158 | 30476098 | + | Island | HORMAD2(tss1500) |
| cg10800953 | 1.84E+08 | − | Island | CHRD(body) |
| cg13477780 | 75897549 | + | S_Shore | SRRM3(body) |
| cg00701706 | 72931632 | + | N_Shore | OTOP3(tss1500) |
| cg01759114 | 56494607 | + | | |
| cg11688949 | 27115956 | + | | STMN4(body) |
| cg10365312 | 43744110 | − | | WFDC5(tss1500) |
| cg26019584 | 1302689 | + | Island | |
| cg00537837 | 85363209 | − | Island | |
| cg01283332 | 1856932 | + | Island | |
| cg18798774 | 1.18E+08 | + | | HSPA12A(body) |
| cg04631458 | 1329462 | − | Island | |
| cg05888252 | 1.76E+08 | + | | |
| cg16894439 | 2.29E+08 | − | S_Shore | |
| cg22172057 | 1405937 | + | Island | ADAR82(body) |
| cg00976097 | 421733 | − | Island | AHRR(body) |
| cg07586026 | 12600225 | − | N_Shore | |
| cg05714552 | 32118295 | − | Island | PRRT1(body) |
| cg26174326 | 26016674 | + | N_Shelf | |
| cg10503232 | 2891495 | − | S_Shore | KCNQ1DN(body) |
| cg27244482 | 1.5E+08 | + | | CAMK2A(tss200) |
| cg00603340 | 1.81E+08 | − | N_Shore | TRIM7(body) |
| cg20958628 | 15249874 | + | N_Shore | KAZN(body); KAZN(tss1500) |
| cg19098932 | 2345152 | + | N_Shore | PEX10(tss1500) |
| cg19590598 | 1.28E+08 | + | Island | |
| cg24024528 | 59478783 | + | | |
| cg02400092 | 1.3E+08 | + | S_Shore | ZBTB44(tss1500) |
| cg10866062 | 72358370 | + | S_Shelf | BTBD17(tss1500) |
| cg23851167 | 62131189 | + | S_Shelf | EEF1A2(tss1500) |
| cg19146112 | 74072255 | − | N_Shore | KHDC3L(tss200) |
| cg18224262 | 56436255 | + | S_Shore | |
| cg17434154 | 2.17E+08 | − | N_Shore | ESRRG(body) |
| cg20091689 | 97524862 | + | S_Shore | ANKRD39(tss1500) |
| cg03521737 | 1.81E+08 | + | | |
| cg27302994 | 30240265 | + | | |
| cg05390307 | 1.25E+08 | + | S_Shore | FEZ1(tss1500) |
| cg24360241 | 2.33E+08 | + | S_Shelf | |
| cg08572336 | 51165404 | + | Island | SHANK1(body) |
| cg06419659 | 1.54E+08 | + | | S100A1(tss1500); S100A13(body); S100A13 |
| | | | | (tss1500); S100A13(tss200) |
| cg14263391 | 37894014 | − | | GRB7(tss1500); GRB7(tss200) |
| cg25763520 | 49687715 | − | N_Shore | PRPH(tss1500) |
| cg14508777 | 42847116 | + | S_Shore | HIGD1A(tss1500) |
| cg04198698 | 62515913 | − | N_Shore | |
| cg24152179 | 19750918 | + | N_Shore | TBX1(body) |
| cg22986999 | 68781976 | + | S_Shelf | MRGPRF(tss1500) |
| cg08421934 | 33942413 | − | N_Shore | |
| cg10202113 | 98520089 | − | S_Shore | |
| cg09636313 | 89070025 | + | N_Shore | |
| cg17382841 | 38445614 | + | | EGFLAM(body) |
| cg02413092 | 1.57E+08 | + | Island | NOM1(tss1500) |
| cg05586134 | 42892994 | − | Island | PTCRA(body) |
| cg10270430 | 34024362 | + | Island | GRM4(body) |
| cg02989255 | 32063774 | + | Island | TNXB(body) |
| cg00337187 | 3054716 | + | | |
| cg07296387 | 44838981 | + | Island | CDH22(body) |
| cg08401657 | 10399046 | + | S_Shore | TFAP2A(body) |
| cg10638827 | 56665855 | − | N_Shore | MT1M(tss1500) |
| cg03546360 | 12675968 | + | N_Shore | DHRS3(body) |
| cg16844333 | 89167457 | − | | LINC00864(body) |
| cg05112555 | 71739385 | + | | LOC100134391(body) |
| cg25075776 | 30848828 | + | N_Shelf | |
| cg09576209 | 2339614 | + | Island | CACNA1C(body) |
| cg05998295 | 21775764 | − | N_Shore | XPO7(tss1500) |
| cg14991769 | 1.5E+08 | − | Island | TMEM176A(tss1500); TMEM176B |
| | | | | (body) |
| cg02605996 | 1.49E+08 | + | | |
| cg05409945 | 52604025 | + | | C12orf80(body); LINC00592(tss1500) |
| cg06201514 | 1817409 | + | | MYT1L(body) |
| cg25944717 | 1.81E+08 | + | N_Shore | TRIM41(tss1500) |
| cg02068015 | 17848581 | + | N_Shore | |
| cg26006084 | 1.72E+08 | + | S_Shore | STK10(tss1500) |
| cg09353289 | 1.03E+08 | + | | |
| cg01020079 | 51236704 | + | Island | TMPRSS12(body) |
| cg02680050 | 1.33E+08 | − | Island | LRCOL1(body) |
| cg05300029 | 21148957 | − | N_Shore | |
| cg03684807 | 46457384 | + | Island | |
| cg25607670 | 1.97E+08 | + | N_Shelf | MFI2(body) |
| cg23445321 | 2.34E+08 | − | Island | SLC35F3(body) |
| cg10714061 | 33714631 | − | | IP6K3(body) |
| cg02996583 | 1.42E+08 | − | Island | SLC45A4(body) |
| cg14769703 | 43818695 | + | S_Shore | |
| cg27051129 | 51417977 | + | Island | DLEU7(tss200); DLEU7-AS1(body) |
| cg23358886 | 2.12E+08 | + | Island | RD3(body) |
| cg14135852 | 1.18E+08 | − | Island | |
| cg19817820 | 1.35E+08 | + | N_Shore | KNDC1(body) |
| cg16228356 | 43848958 | + | | CRHR1(body) |
| cg02865928 | 47983346 | − | S_Shelf | |
| cg26298737 | 2.26E+08 | + | N_Shore | SRP9(tss1500) |
| cg24690094 | 67383802 | − | Island | DOC2GP(tss1500) |
| cg25423077 | 59221601 | + | Island | CDH20(body) |
| cg21211367 | 1.62E+08 | − | N_Shore | |
| cg00333946 | 99975310 | − | | |
| cg08632909 | 70048796 | + | N_Shore | FADD(tss1500) |
| cg11426075 | 8086959 | − | S_Shore | ERRFI1(tss1500) |
| cg18025886 | 1.97E+08 | − | N_Shelf | MFI2(body) |
| cg00270625 | 51017019 | − | Island | CHKB- |
| | | | | CPT1B(body); CPT1B(body); CPT1B |
| | | | | (tss1500); CPT1B(tss200) |
| cg03122247 | 1.71E+08 | − | N_Shore | |
| cg22222281 | 6308758 | − | N_Shore | CD9(tss1500) |
| cg20785560 | 78511235 | − | N_Shore | GIPC2(tss1500) |
| cg12684052 | 19883602 | + | Island | |
| cg13016619 | 527786 | − | | |
| cg00178928 | 2.33E+08 | − | N_Shore | |
| cg03569507 | 46114237 | + | N_Shore | COPZ2(body) |
| cg06572093 | 69095231 | − | Island | |
| cg18046394 | 29924165 | + | Island | WIPF3(body) |
| cg09168805 | 94514548 | − | | ABCA4(body) |
| cg23907108 | 64405993 | + | Island | NRXN2(body) |
| cg15414745 | 74064047 | + | Island | DPPA5(tss200) |
| cg09798888 | 1.08E+08 | + | S_Shore | |
| cg16096531 | 53722216 | + | S_Shelf | SP7(body) |
| cg22360016 | 1.05E+08 | − | S_Shore | |
| cg04312620 | 50419214 | − | S_Shore | SALL4(tss200) |
| cg25974617 | 71520962 | − | S_Shore | LOC286190(body); TRAM1(tss1500) |
| cg09693926 | 1.79E+08 | + | Island | ADAMTS2(body) |
| cg14818812 | 1.42E+08 | + | | |
| cg16284953 | 11471571 | + | S_Shore | |
| cg06042504 | 55087323 | − | | |
| cg25402787 | 53084679 | + | N_Shore | |
| cg26164577 | 96251788 | + | N_Shore | SNRPF(tss1500) |
| cg22160612 | 45705042 | + | N_Shore | FAM118A(tss1500); FAM118A(tss200) |
| cg23052055 | 3606042 | − | N_Shore | TP73(body); TP73(tss1500) |
| cg19228034 | 92672812 | − | Island | |
| cg08818632 | 72931569 | − | N_Shore | OTOP3(tss1500) |
| cg04037970 | 54955965 | + | N_Shore | |
| cg25903143 | 68920466 | − | | LOC338694(body) |
| cg20480274 | 27313125 | + | Island | SEZ6(body) |
| cg06588556 | 18477299 | + | Island | LDHAL6A(tss200) |
| cg26537280 | 95699037 | − | N_Shore | RWDD3(tss1500); TMEM56- |
| | | | | RWDD3(body) |
| cg06590170 | 33969828 | − | | |
| cg25502179 | 46875342 | + | Island | PRSS42(body) |
| cg21872908 | 55271673 | − | Island | C1orf17(tss200) |
| cg19645200 | 1.8E+08 | + | N_Shore | |
| cg00787996 | 30072616 | + | S_Shore | TRIM31(body) |
| cg15138125 | 1.58E+08 | + | Island | |
| cg13796250 | 1.08E+08 | − | N_Shore | |
| cg21209395 | 35406764 | − | | ATP8B5P(body); ATP8B5P(tss200) |
| cg14995475 | 6534774 | − | Island | TNFSF9(body) |
| cg07115262 | 52702819 | − | S_Shore | KRT86(body) |
| cg13970894 | 1.35E+08 | + | N_Shore | |
| cg10092257 | 19476959 | + | Island | |
| cg04624413 | 41504763 | − | S_Shore | NKX6-3(body) |
| cg26664752 | 1.24E+08 | + | | |
| cg25879142 | 4671391 | + | | |
| cg09482093 | 19314328 | − | | RNF112(tss200) |
| cg03043822 | 25160098 | + | Island | PIWIL3(body); TOPIP2(tss1500) |
| cg06978117 | 1.11E+08 | + | | C11orf53(body) |
| cg13526469 | 13245693 | + | Island | |
| cg12291059 | 19997924 | + | | CTAGE1(tss200) |
| cg22047282 | 27301649 | + | N_Shelf | EMILIN1(body) |
| cg02905426 | 28051771 | − | N_Shore | FAM76A(tss1500) |
| cg18713687 | 1.95E+08 | − | Island | MUC4(body) |
| cg03533472 | 46919112 | − | S_Shore | GPT2(body) |
| cg23217153 | 2.43E+08 | − | Island | |
| cg01477942 | 2978022 | + | | |
| cg19928597 | 73157383 | + | S_Shelf | |
| cg07130150 | 96883863 | + | S_Shore | |
| cg18256205 | 1.81E+08 | + | N_Shore | |
| cg05351887 | 3988869 | − | N_Shore | |
| cg01786973 | 1.27E+08 | + | Island | NR5A1(body) |
| cg01693350 | 32452187 | + | Island | WT1(body) |
| cg02152233 | 29189789 | + | Island | OPRD1(body) |
| cg14058476 | 24835609 | − | N_Shore | NFATC4(tss1500) |
| cg04589008 | 660692 | + | Island | |
| cg04229928 | 1.19E+08 | − | | USP2-AS1(body) |
| cg19193384 | 30244184 | − | Island | |
| cg04933168 | 36960937 | − | Island | CACNG2(body) |
| cg10783149 | 55707866 | + | | |
| cg25362652 | 1.06E+08 | + | Island | |
| cg21052104 | 1.79E+08 | − | | ADAMTS2(body) |
| cg19774782 | 1.24E+08 | − | | TACC2(body) |
| cg14286514 | 32525315 | − | N_Shore | DDX58(body) |
| cg16695380 | 11743020 | − | S_Shore | MAD2L2(body) |
| cg13521842 | 1.01E+08 | + | S_Shore | BEGAIN(tss200) |
| cg12763978 | 28601519 | + | N_Shore | |
| cg20740051 | 1.61E+08 | − | S_Shore | B4GALT3(body) |
| cg21784396 | 1.28E+08 | − | Island | PRRT4(body) |
| cg14089881 | 6296630 | + | S_Shore | ICMT(tss1500); LINC00337(body) |
| cg24667603 | 96590566 | − | | |
| cg10394898 | 69755745 | + | | |
| cg12748639 | 71035898 | + | | CLEC4F(body) |
| cg04945076 | 1.31E+08 | + | | |
| cg06979680 | 87676527 | − | | GRID1(body) |
| cg27529555 | 2.05E+08 | − | | KLHDC8A(body) |
| cg14254433 | 34482411 | + | | PACSIN1(body); PACSIN1(tss1500) |
| cg20761187 | 1.34E+08 | − | | LOC100128239(tss1500) |
| cg17818731 | 43295753 | + | S_Shore | |
| cg23740940 | 68924746 | + | Island | LOC338694(body) |
| cg03209412 | 1.84E+08 | − | Island | |
| cg09791746 | 1510494 | + | Island | ADAMTSLS(body) |
| cg11060441 | 1.49E+08 | − | N_Shore | SSPO(body) |
| cg12838168 | 1.94E+08 | + | | LRRC15(body) |
| cg11541486 | 72358055 | − | S_Shore | BTBD17(tss200) |
| cg17289378 | 1.69E+08 | + | S_Shelf | |
| cg02855836 | 1.17E+08 | − | Island | |
| cg27367526 | 89841692 | + | S_Shore | STEAP2(body); STEAP2(tss1500) |
| cg24123198 | 1.84E+08 | + | N_Shore | |
| cg20128319 | 97174815 | + | S_Shore | NEURL3(tss1500) |
| cg14521421 | 8862019 | − | | ST5(body) |
| cg05671968 | 1.58E+08 | − | | |
| cg27475169 | 1.19E+08 | + | N_Shore | |
| cg26656672 | 1.64E+08 | + | Island | |
| cg04462774 | 50368110 | + | | AQP6(body) |
| cg18478731 | 1.3E+08 | − | Island | |
| cg08828723 | 32165176 | − | S_Shore | NOTCH4(body) |
| cg00933542 | 1.5E+08 | − | N_Shore | PCMT1(tss1500) |
| cg15325070 | 2792704 | − | | |
| cg23012917 | 2.29E+08 | − | Island | |
| cg14502850 | 49731991 | − | S_Shore | C1QL4(tss1500) |
| cg01066936 | 41776843 | + | N_Shore | TEF(body); TEF(tss1500) |
| cg23164850 | 79495348 | + | Island | FSCN2(tss200) |
| cg16180796 | 23245900 | − | Island | |
| cg13930544 | 2.48E+08 | − | Island | GCSAML(body); GCSAML- |
| | | | | AS1(body); OR2C3(body) |
| cg09933329 | 44186183 | + | Island | GCK(body) |
| cg08817693 | 1.07E+08 | + | N_Shore | C12orf23(tss1500) |
| cg00927699 | 46493289 | + | N_Shore | ADARB1(tss1500); SSR4P1(tss200) |
| cg17100943 | 6459300 | − | Island | SLC25A23(body) |
| cg00639837 | 20711637 | + | S_Shore | |
| cg02773436 | 50650248 | − | | |
| cg19027710 | 1.43E+08 | + | N_Shore | CHST2(tss1500) |
| cg23791325 | 95743038 | + | S_Shore | |
| cg18163342 | 1.05E+08 | − | Island | CEP170B(body) |
| cg20292791 | 58089357 | + | | DAB1(body) |
| cg24996814 | 68417376 | + | | |
| cg06282802 | 1.28E+08 | + | | |
| cg05589489 | 89748821 | − | S_Shore | |
| cg09084279 | 79495006 | − | Island | FSCN2(tss1500) |
| cg01801826 | 1766281 | + | S_Shore | |
| cg03001867 | 47047897 | + | Island | |
| cg20790798 | 1857306 | + | Island | |
| cg02899903 | 3216063 | + | S_Shore | PITRM1(tss1500) |
| cg11872743 | 48858651 | − | Island | |
| cg25599129 | 1.16E+08 | + | | |
| cg16885093 | 61988297 | + | | CSHL1(body) |
| cg23388085 | 1595741 | + | N_Shelf | |
| cg15197065 | 78985562 | − | | CMYA5(tss200) |
| cg02940147 | 1.25E+08 | + | S_Shore | ROBO3(body) |
| cg10770023 | 51016644 | − | Island | CHKB- |
| | | | | CPT1B(body); CPT1B(body); CPT1B |
| | | | | (tss200) |
| cg20171429 | 89873821 | + | N_Shore | C7orf63(tss1500) |
| cg18038544 | 14576099 | − | | GRIP2(body) |
| cg23688479 | 42756851 | − | | CCDC152(tss200) |
| cg05877550 | 85369038 | − | | |
| cg18201671 | 2.23E+08 | − | S_Shore | TLR5(tss1500) |
| cg05180717 | 34516488 | − | | EMC4(tss1500) |
| cg14076971 | 2137665 | + | S_Shore | C1orf86(body) |
| cg12743470 | 74815085 | − | | VRTN(tss200) |
| cg15015340 | 61486220 | + | N_Shore | DAGLA(body) |
| cg13023646 | 1.66E+08 | − | | TRIM61(tss200) |
| cg05467458 | 33361032 | − | | SLC7A9(tss1500) |
| cg23098371 | 92012061 | + | Island | |
| cg17628249 | 58499854 | − | S_Shore | C17orf64(tss200) |
| cg02616418 | 1.54E+08 | − | | TRIM2(body) |
| cg11400650 | 69328155 | − | S_Shelf | MIR548H4(body); NOX5(body) |
| cg06950634 | 304351 | + | Island | |
| cg13639937 | 92012655 | + | Island | |
| cg00782200 | 1.45E+08 | + | N_Shore | |
| cg15780361 | 2.02E+08 | + | S_Shore | ALS2CR11(tss200) |
| cg14531437 | 1.13E+08 | + | S_Shelf | |
| cg17804348 | 3649473 | + | Island | TP73(body) |
| cg00493304 | 48844908 | + | Island | GTF2A1L(tss200); STON1- |
| | | | | GTF2A1L(body) |
| cg22897224 | 25160406 | − | S_Shore | PIWIL3(body); TOP1P2(tss200) |
| cg23009263 | 1.01E+08 | + | | |
| cg10053622 | 7643808 | − | Island | DNAH2(body) |
| cg00412675 | 11577758 | − | | GATA4(body) |
| cg06642945 | 1.64E+08 | + | S_Shore | PACRG(body) |
| cg01924435 | 1.85E+08 | − | Island | |
| cg08289350 | 74524371 | − | | CYGB(body); PRCD(body) |
| cg20949251 | 7432666 | − | | |
| cg05474265 | 81128398 | + | Island | |
| cg03309770 | 10912478 | − | Island | TVP23A(body) |
| cg14333454 | 27189298 | − | N_Shore | SFN(tss1500) |
| cg02930419 | 19628087 | + | Island | |
| cg06221946 | 32802312 | + | S_Shore | MARCKSL1(tss1500) |
| cg16876823 | 2987082 | − | | TLE6(body) |
| cg15974931 | 1.1E+08 | + | N_Shore | PKHD1L1(tss1500) |
| cg24557048 | 2063799 | − | Island | PRKCZ(body) |
| cg02064284 | 1.03E+08 | + | | |
| cg07626700 | 1.57E+08 | + | Island | NOM1(tss1500) |
| cg16442587 | 56514573 | + | | |
| cg19865237 | 1.05E+08 | − | Island | |
| cg11855943 | 47964664 | − | N_Shelf | |
| cg02479842 | 11123536 | + | | |
| cg12723492 | 1.27E+08 | − | | KIRREL3(body) |
| cg16454316 | 32847845 | + | Island | |
| cg22937320 | 19032778 | − | | FAM154A(body) |
| cg20262330 | 70826997 | + | S_Shore | COX16(tss1500); SYNJ2BP- |
| | | | | COX16(body) |
| cg08572513 | 1.26E+08 | − | | KIRREL3(body) |
| cg03382250 | 1.26E+08 | − | | KIRREL3(body) |
| cg05316463 | 95786663 | + | S_Shore | CLMN(tss1500) |
| cg22928954 | 56061106 | − | N_Shore | |
| cg17545182 | 2.48E+08 | − | Island | GCSAML(body); GCSAML- |
| | | | | AS1(body); OR2C3(body) |
| cg25104872 | 28828977 | − | N_Shelf | LOC401242(body) |
| cg10962524 | 83017009 | + | N_Shore | HAPLN1(tss200) |
| cg11709544 | 26233404 | + | Island | MIR3917(tss1500); STMN1(tss1500); |
| | | | | STMN1(tss200) |
| cg09893305 | 83017184 | − | N_Shore | HAPLN1(tss1500) |
| cg18335290 | 1.28E+08 | + | | LIMS2(body) |
| cg12912758 | 1594591 | − | | |
| cg05063670 | 1.67E+08 | − | | RPS6KA2(body) |
| cg03142554 | 82958230 | + | S_Shore | IBTK(tss1500) |
| cg08370173 | 75537444 | − | | |
| cg23549902 | 5184155 | + | Island | ZNF890P(body) |
| cg03800065 | 1.35E+08 | + | S_Shore | |
| cg03880033 | 55866757 | + | Island | CES1(body) |
| cg14836522 | 31375133 | + | | SDC3(body) |
| cg21193577 | 1.01E+08 | + | S_Shore | BEGAIN(tss200) |
| cg10982664 | 78407418 | − | | BHMT(tss200) |
| cg16074228 | 74072261 | − | N_Shore | KHDC3L(tss200) |
| cg27179680 | 2.05E+08 | − | | |
| cg23052855 | 1.96E+08 | + | | ZDHHC19(body) |
| cg04017512 | 35195138 | + | | |
| cg01968133 | 10711542 | − | N_Shelf | CASZ1(body) |
| cg00658082 | 74669380 | + | Island | RTKN(tss1500) |
| cg02978140 | 23880736 | + | N_Shore | |
| cg04342483 | 7259290 | + | | CAMTA1(body) |
| cg08548500 | 1.14E+08 | + | | F10(body) |
| cg08159989 | 2.05E+08 | + | | KLHDC8A(body) |
| cg15246511 | 21267212 | + | S_Shore | APOB(tss1500) |
| cg04140525 | 1.53E+08 | + | | |
| cg16428357 | 34190258 | − | Island | |
| cg07013104 | 73357095 | + | | PLEKHB1(tss1500); PLEKHB1(tss200) |
| cg03883572 | 66461884 | − | S_Shelf | MAST4(body) |
| cg25924688 | 72977851 | − | N_Shore | BBS4(tss1500); HIGD2B(body) |
| cg25396728 | 248500 | + | Island | IQSEC3(body); LOC574538(body) |
| cg18941641 | 78392320 | + | | |
| cg07689920 | 73356316 | − | | PLEKHB1(tss1500) |
| cg00398420 | 56681867 | − | | |
| cg14962509 | 36039655 | + | Island | TFAP2E(body) |
| cg05017199 | 1.5E+08 | + | Island | TMEM176A(tss1500); TMEM176B |
| | | | | (body) |
| cg14958041 | 95675546 | − | Island | |
| cg16844661 | 2188244 | + | Island | TH(body) |
| cg15774065 | 79495320 | − | Island | FSCN2(tss200) |
| cg02831900 | 1.84E+08 | + | S_Shore | |
| cg10807643 | 55907417 | − | Island | TBPL2(tss200) |
| cg08288433 | 20799511 | + | Island | CCDC144NL(tss200); LOC440416(body) |
| cg02769477 | 17012728 | + | | |
| cg18288532 | 90597356 | + | Island | |
| cg02174341 | 13276466 | + | | UCMA(tss200) |
| cg03579045 | 11579013 | − | | GATA4(body) |
| cg07779699 | 55873983 | + | Island | FAM71E2(body) |
| cg11334165 | 1.46E+08 | + | Island | |
| cg24635754 | 1.17E+08 | + | | |
| cg01027976 | 42734612 | + | Island | |
| cg18660345 | 35169539 | + | Island | MYL9(tss1500) |
| cg14485097 | 4671479 | + | | |
| cg08878450 | 36915864 | − | Island | OSCP1(body) |
| cg27168291 | 33282997 | + | Island | TAPBP(tss1500); ZBTB22(body) |
| cg17520215 | 1.01E+08 | − | N_Shore | GAS2L3(tss1500) |
| cg00314115 | 1.21E+08 | − | Island | |
| cg05373875 | 18485171 | − | Island | |
| cg05369327 | 95826853 | + | Island | |
| cg16677399 | 28830902 | + | N_Shore | LOC401242(body) |
| cg03302546 | 1.12E+08 | − | N_Shore | |
| cg10359157 | 51418020 | − | Island | DLEU7(tss200); DLEU7-AS1(body) |
| cg27128941 | 1.78E+08 | − | Island | |
| cg10519417 | 14201321 | + | N_Shore | |
| cg12606933 | 1.57E+08 | + | Island | SOX30(body) |
| cg18498622 | 1.52E+08 | + | S_Shelf | GABRQ(body) |
| cg06993467 | 21966500 | + | | RAP1GAP(body) |
| cg11966998 | 15692519 | − | | |
| cg19520234 | 1.41E+08 | − | | LOC152586(body) |
| cg03012289 | 1.1E+08 | − | N_Shore | PKHD1L1(tss1500) |
| cg13731640 | 5278467 | + | | |
| cg00907008 | 350892 | + | Island | CHL1(body) |
| cg26607337 | 1.46E+08 | + | Island | |
| cg03549506 | 45695008 | + | S_Shore | |
| cg25005368 | 47967085 | − | N_Shore | |
| cg23008871 | 45949743 | − | Island | |
| cg06915226 | 70960959 | + | | ADD2(body) |
| cg21849552 | 14597650 | − | Island | |
| cg24202119 | 35938692 | − | | CAPSL(body) |
| cg06894628 | 1.44E+08 | − | S_Shore | SLURP1(body) |
| cg06269928 | 2.42E+08 | + | Island | |
| cg14803869 | 1.31E+08 | + | | |
| cg08209422 | 39407567 | + | | RHBDL2(tss200) |
| cg02458053 | 47061442 | + | N_Shore | |
| cg04724659 | 2.29E+08 | + | S_Shore | CCSAP(tss1500) |
| cg10166889 | 61028374 | − | | |
| cg04105712 | 1.58E+08 | + | S_Shelf | |
| cg18943588 | 7517833 | + | | |
| cg25943719 | 1.01E+08 | − | S_Shore | ALDH1A3(body) |
| cg27083329 | 29210949 | − | | |
| cg27629948 | 89285170 | − | Island | |
| cg19870370 | 21010223 | − | Island | CRYL1(body) |
| cg10570754 | 5589444 | + | | NTF3(body) |
| cg05030553 | 44061403 | + | | |
| cg11556592 | 79283300 | − | N_Shore | LINC00482(tss1500) |
| cg02477603 | 1.25E+08 | + | | |
| cg23913963 | 17766917 | + | S_Shore | RCC2(tss1500) |
| cg24253618 | 32054460 | − | Island | |
| cg07897069 | 1.27E+08 | + | N_Shore | |
| cg19764594 | 10806288 | + | | LINC00606(tss1500) |
| cg12439325 | 51236768 | + | Island | TMPRSS12(body) |
| cg24756378 | 33401638 | + | N_Shore | |
| cg00627029 | 72322791 | + | S_Shore | KIF19(body) |
| cg07636176 | 21267175 | − | S_Shore | APOB(tss1500) |
| cg03472880 | 1.77E+08 | + | N_Shore | |
| cg13227481 | 2.2E+08 | − | N_Shore | DES(tss200) |
| cg09239106 | 49937997 | − | | KCNH3(body) |
| cg02961934 | 1.51E+08 | + | | |
| cg09517075 | 22133004 | − | Island | PIWIL2(body); PIWIL2(tss200) |
| cg10742957 | 39466716 | + | S_Shore | FBXO17(tss1500) |
| cg22933058 | 43472972 | + | Island | |
| cg16816874 | 11578850 | − | | GATA4(body) |
| cg18145090 | 20805094 | − | N_Shore | GJB6(body) |
| cg27166581 | 1.55E+08 | − | S_Shore | |
| cg13723983 | 1.32E+08 | + | S_Shore | C9orf50(tss1500); NTMT1(body) |
| cg16331632 | 60783238 | − | | |
| cg16568641 | 1.34E+08 | − | S_Shore | LRRC6(tss1500) |
| cg00806138 | 47881404 | − | N_Shore | FOXE3(tss1500) |
| cg06168149 | 50497778 | − | | GPD1(body) |
| cg06928246 | 2.28E+08 | − | Island | |
| cg07011961 | 23507594 | + | | PRDM9(tss200) |
| cg00693419 | 79325320 | + | | KCNMA1(body) |
| cg23639734 | 24796753 | − | | SOD3(tss1500) |
| cg14414792 | 69992001 | + | S_Shore | ATOH7(tss200) |
| cg02931685 | 14376360 | − | N_Shore | |
| cg09889948 | 48858873 | + | Island | |
| cg02052758 | 55430948 | + | N_Shelf | |
| cg07238058 | 19936611 | − | | |
| cg26817546 | 1.36E+08 | + | | |
| cg25154244 | 1.35E+08 | + | | |
| cg10401489 | 513241 | + | | TPGS1(body) |
| cg03604763 | 85527490 | − | | WDR63(tss1500) |
| cg08932654 | 38084459 | + | S_Shore | ORMDL3(tss1500) |
| cg17515480 | 48049039 | − | Island | ZNF541(body) |
| cg03217115 | 1.19E+08 | + | N_Shore | |
| cg08891559 | 1.19E+08 | − | | |
| cg25878149 | 1.5E+08 | − | Island | |
| cg23737263 | 43328424 | − | S_Shore | |
| cg19159759 | 20989818 | − | Island | CRYL1(body) |
| cg14010405 | 89357911 | − | S_Shore | GTF2B(tss1500) |
| cg04248271 | 1.59E+08 | + | Island | EBF1(body) |
| cg07075347 | 44104860 | + | Island | PGAM2(body) |
| cg13017614 | 47881737 | − | N_Shore | FOXE3(tss200) |
| cg04850999 | 77872084 | − | Island | C10orf11(body) |
| cg08111158 | 2.28E+08 | + | Island | |
| cg05661164 | 52715043 | − | Island | KRT83(body) |
| cg26039829 | 22132926 | − | Island | PIWIL2(body); PIWIL2(tss200) |
| cg21467935 | 31781906 | − | N_Shore | HSPA1A(tss1500); HSPA1L(body) |
| cg02931604 | 74064298 | − | Island | DPPA5(tss1500) |
| cg24083746 | 45826152 | − | N_Shore | CKM(body) |
| cg10574365 | 32054836 | + | Island | |
| cg07865444 | 47866659 | − | Island | |
| cg24945462 | 46714985 | + | Island | LOC642852(body) |
| cg17000480 | 1.35E+08 | + | S_Shore | TTC40(tss1500) |
| cg08888203 | 10149979 | − | Island | FANCD2OS(tss200) |
| cg06070578 | 1.03E+08 | − | | |
| cg02042585 | 1.61E+08 | − | Island | SLC22A3(body) |
| cg22374525 | 1.16E+08 | + | N_Shore | |
| cg07394701 | 1.45E+08 | − | S_Shore | LOC100288142(body); NBPF10(body) |
| cg11231735 | 2.38E+08 | + | | |
| cg17129388 | 47571986 | + | N_Shore | NGFR(tss1500) |
| cg00001099 | 87081553 | + | N_Shore | PSKH2(body) |
| cg14062977 | 1.35E+08 | + | | C9orf171(body) |
| cg05157195 | 50820135 | − | S_Shore | |
| cg24932660 | 27709575 | + | N_Shore | CD164L2(body) |
| cg01570972 | 10090656 | + | N_Shore | GRHL1(tss1500) |
| cg15677087 | 61584850 | − | S_Shore | SLC17A9(body) |
| cg02707176 | 1.41E+08 | + | Island | PCDHGA1(body); PCDHGA10(body); |
| | | | | PCDHGA2(body); PCDHGA3(body); |
| | | | | PCDHGA4(body); PCDHGA5(body); |
| | | | | PCDHGA6(body); PCDHGA7(body); |
| | | | | PCDHGA8(body); PCDHGA9(body); |
| | | | | PCDHGB1(body); PCDHGB2(body); |
| | | | | PCDHGB3(body); PCDHGB4(body); |
| | | | | PCDHGB5(body); PCDHGB6(body); |
| | | | | PCDHGB7(body) |
| cg02197228 | 2.2E+08 | + | N_Shore | ASIC4(body) |
| cg14307477 | 51330469 | − | Island | KLK15(body) |
| cg08310581 | 1.32E+08 | − | Island | ARHGEF4(body) |
| cg03295928 | 74669349 | + | Island | RTKN(tss1500) |
| cg09652312 | 1.55E+08 | − | Island | |
| cg22745354 | 928720 | − | | |
| cg08246800 | 73356353 | − | | PLEKHB1(tss1500) |
| cg07045089 | 3620218 | − | N_Shelf | TP73(body) |
| cg27350104 | 70958638 | − | N_Shelf | |
| cg19392379 | 1.24E+08 | − | | TACC2(body) |
| cg17519938 | 3713580 | − | S_Shore | LRRC47(tss1500) |
| cg09853936 | 3388196 | − | | ARHGEF16(body) |
| cg10872641 | 91510597 | − | S_Shore | MTERF(tss1500) |
| cg15117754 | 10150083 | + | S_Shore | FANCD2OS(tss200) |
| cg19297232 | 68483021 | + | S_Shore | SMPD3(tss1500) |
| cg25712005 | 73443113 | − | | ELN(body) |
| cg25301173 | 1.91E+08 | + | | |
| cg07745344 | 514802 | − | | |
| cg07651048 | 49735444 | + | N_Shore | |
| cg12101586 | 75019203 | + | Island | CYP1A1(tss1500) |
| cg14299483 | 1.02E+08 | + | | |
| cg05985317 | 32118208 | − | Island | PRRT1(body) |
| cg19605931 | 8662653 | − | S_Shelf | |
| cg01803844 | 8036816 | − | | |
| cg26820168 | 1.24E+08 | − | N_Shore | FAM83A(body) |
| cg26586027 | 12068363 | − | Island | |
| cg22700246 | 99978986 | − | | |
| cg09590377 | 8597389 | − | Island | |
| cg04452896 | 1.24E+08 | + | | |
| cg17391620 | 31734471 | + | | VWA7(body) |
| cg18052665 | 74064140 | + | Island | DPPA5(tss200) |
| cg15139452 | 1.76E+08 | − | | |
| cg18318818 | 48640257 | − | S_Shore | CACNA1G(body); CACNA1G- |
| | | | | AS1(tss1500) |
| cg22199969 | 2.33E+08 | + | Island | ALPI(body) |
| cg10403849 | 70321959 | − | S_Shore | TET1(body) |
| cg03483944 | 55874700 | − | S_Shore | FAM71E2(tss200) |
| cg24363820 | 51016703 | − | Island | CHKB- |
| | | | | CPT1B(body); CPT1B(body); CPT1B |
| | | | | (tss200) |
| cg00448720 | 24883014 | + | S_Shore | C1QTNF9(tss1500) |
| cg25137841 | 31536585 | − | | PLA2G3(tss200) |
| cg26172342 | 41654331 | − | N_Shore | ANK1(body) |
| cg26343510 | 72975668 | − | Island | P2RY6(body); P2RY6(tss1500) |
| cg21593409 | 88706389 | + | Island | IL17C(body) |
| cg06222062 | 20396690 | − | | PLA2G5(tss200) |
| cg19309616 | 41851836 | − | S_Shelf | |
| cg26940100 | 2.28E+08 | − | N_Shelf | C1orf145(body); OBSCN(tss200) |
| cg11857646 | 1.51E+08 | − | Island | KCNH2(body) |
| cg19602315 | 30669494 | + | S_Shore | C17orf75(tss1500) |
| cg16970604 | 61325803 | + | | LOC100505532(body) |
| cg18709881 | 72837627 | + | Island | |
| cg08659204 | 35242555 | − | | |
| cg06200514 | 7052239 | + | | NLRP14(body) |
| cg08707536 | 33797086 | + | | |
| cg01202519 | 85645743 | − | Island | |
| cg27016974 | 46875724 | + | Island | PRSS42(tss200) |
| cg03996861 | 54605613 | + | | |
| cg21484547 | 28319144 | + | | |
| cg02744588 | 48823880 | + | N_Shore | CCDC114(tss1500) |
| cg14213394 | 38508585 | + | Island | |
| cg20317418 | 93679543 | − | | |
| cg17344906 | 13202507 | − | S_Shelf | NFIX(body) |
| cg21144493 | 7130061 | − | | CAMTA1(body) |
| cg08767820 | 51539583 | − | | |
| cg12626589 | 32120783 | + | N_Shore | LOC100507547(body); PPT2(tss1500); |
| | | | | PPT2- |
| | | | | EGFL8(tss1500); PRRT1(tss1500) |
| cg16877087 | 1.62E+08 | + | | |
| cg04518195 | 51020534 | + | Island | LRRC4B(body) |
| cg04833011 | 98836673 | + | | |
| cg25686793 | 48342875 | − | | CRX(body) |
| cg13570656 | 75019196 | + | Island | CYP1A1(tss1500) |
| cg04613258 | 47589594 | + | N_Shore | LOC100288866(body); NGFR(body) |
| cg20401945 | 29912460 | + | N_Shore | ASPHD1(body) |
| cg11883836 | 1.57E+08 | − | Island | LINC00880(body) |
| cg25534953 | 97174206 | + | Island | NEURL3(tss1500) |
| cg08374687 | 1.23E+08 | + | Island | ADAD1(body) |
| cg15278109 | 29214117 | + | Island | |
| cg19535358 | 1.13E+08 | + | Island | |
| cg21749424 | 1.66E+08 | − | N_Shelf | |
| cg21867110 | 22700397 | − | Island | |
| cg05828191 | 75019382 | − | S_Shore | WDR59(tss1500) |
| cg11146935 | 30336678 | − | Island | |
| cg27547703 | 1.16E+08 | − | Island | TDRD1(tss200) |
| cg01430344 | 16154047 | + | Island | |
| cg04205744 | 86606367 | + | N_Shelf | |
| cg25612391 | 19216451 | − | | SLC25A42(body) |
| cg18434354 | 1.21E+08 | − | | LINC01101(tss200) |
| cg03333149 | 52473731 | − | Island | |
| cg19197440 | 1811269 | + | N_Shore | |
| cg10687107 | 67454667 | + | Island | |
| cg01401534 | 4079342 | − | Island | C6orf201(tss200); FAM217A(body) |
| cg06637774 | 72975702 | − | Island | P2RY6(body); P2RY6(tss1500) |
| cg15813594 | 38445563 | + | | EGFLAM(body); EGFLAM(tss200) |
| cg02452639 | 1.17E+08 | − | Island | |
| cg25120325 | 1.18E+08 | − | | PNLIPRP2(tss200) |
| cg27108424 | 75537017 | + | | |
| cg27649194 | 1974168 | + | Island | CACNA2D4(body) |
| cg24489344 | 46457998 | + | Island | |
| cg05365033 | 18889003 | − | S_Shore | CRTC1(body) |
| cg08221781 | 66822904 | − | N_Shore | RHOD(tss1500) |
| cg04865929 | 42908410 | + | S_Shore | GJC1(tss1500) |
| cg14080129 | 90439478 | + | N_Shore | |
| cg26706187 | 1.15E+08 | − | Island | |
| cg21067023 | 501513 | − | S_Shore | COLEC12(tss1500) |
| cg03539001 | 79164714 | − | N_Shore | MORF4L1(tss1500) |
| cg19461383 | 69325362 | + | Island | MIR548H4(body); NOX5(body) |
| cg15620905 | 44024150 | − | | PTPRF(body) |
| cg12124927 | 1.2E+08 | + | N_Shore | GRIK4(body); GRIK4(tss1500) |
| cg06521653 | 85845811 | − | Island | |
| cg19884634 | 1.28E+08 | + | S_Shore | PRRT4(body) |
| cg14395298 | 23412250 | − | Island | |
| cg19364351 | 6291879 | − | N_Shore | CCKBR(body) |
| cg10052908 | 74063522 | + | Island | DPPA5(body) |
| cg00206639 | 99987871 | + | | |
| cg19727499 | 49628740 | + | N_Shelf | PPFIA3(body) |
| cg20512303 | 1.32E+08 | − | N_Shore | POLIM4(tss1500) |
| cg00660384 | 43746736 | − | | RASGEF1A(body) |
| cg22276371 | 2.33E+08 | + | S_Shore | CHRND(tss200) |
| cg11976911 | 13509032 | + | | |
| cg10934807 | 1.13E+08 | − | S_Shore | RGPD5(tss1500); RGPD8(tss1500) |
| cg26333618 | 48336655 | + | | |
| cg17263206 | 1.54E+08 | + | S_Shore | |
| cg10161743 | 48917816 | + | N_Shore | GRIN2D(body) |
| cg13809236 | 45908036 | − | S_Shore | MAPK8IP1(body) |
| cg00506049 | 75887380 | − | N_Shore | SRRM3(body) |
| cg08701183 | 21209781 | + | | |
| cg03987199 | 29189655 | + | Island | OPRD1(body) |
| cg10892403 | 62676929 | + | | CHRM1(body) |
| cg26643476 | 1.23E+08 | + | | HCAR1(tss1500) |
| cg03236170 | 87915396 | − | N_Shore | |
| cg24639679 | 32847590 | − | Island | |
| cg02716646 | 25384293 | + | Island | POMC(body) |
| cg00732378 | 55021073 | + | | |
| cg08772003 | 1.05E+08 | + | S_Shore | AS3MT(body); C10orf32- |
| | | | | ASMT(body) |
| cg08966208 | 1769409 | + | Island | IFITM10(body); MOB2(body) |
| cg25101936 | 1.14E+08 | − | N_Shore | ZBTB16(tss1500) |
| cg13010014 | 1.25E+08 | − | S_Shore | ROBO3(body) |
| cg11884274 | 32164927 | − | S_Shore | NOTCH4(body) |
| cg13228642 | 1.81E+08 | − | N_Shore | IER5(tss1500) |
| cg16836311 | 25944712 | + | Island | MAN1C1(body) |
| cg22795769 | 1396123 | − | S_Shore | MYO1C(tss1500); MYO1C(tss200) |
| cg08656770 | 1.17E+08 | − | | |
| cg08274552 | 2.2E+08 | − | N_Shore | SLC4A3(tss1500) |
| cg24892948 | 36001897 | − | | DMKN(body); DMKN(tss1500) |
| cg07803814 | 11746226 | + | N_Shore | VGLL4(body) |
| cg19263607 | 1.4E+08 | − | Island | |
| cg04598670 | 68697651 | − | | |
| cg16129526 | 69968273 | − | | PLEKHD1(body) |
| cg12686920 | 41739806 | + | | MEOX1(tss1500) |
| cg26841862 | 1.33E+08 | − | Island | |
| cg08538032 | 50574507 | − | | ELAVL4(body); ELAVL4(tss200) |
| cg00324725 | 56581212 | + | | |
| cg15468668 | 7082699 | + | | ASGR1(body) |
| cg11024722 | 5406901 | + | | UCN3(tss200) |
| cg22382021 | 1.46E+08 | − | Island | ARHGAP39(body); C8orf82(tss1500) |
| cg26396721 | 214171 | − | Island | |
| cg25883020 | 41504993 | − | S_Shore | NKX6-3(tss200) |
| cg11262850 | 1.54E+08 | + | S_Shore | |
| cg24580146 | 7644241 | − | Island | DNAH2(body) |
| cg03162994 | 23713729 | − | Island | PSMA8(tss200) |
| cg00316312 | 1.35E+08 | − | N_Shelf | GPR123(body) |
| cg24184022 | 55431084 | − | N_Shelf | |
| cg05767720 | 32117281 | + | N_Shore | PRRT1(body) |
| cg19403023 | 2848797 | − | Island | PRSS41(body) |
| cg20699586 | 2.28E+08 | − | Island | |
| cg08358943 | 44889584 | − | | WNT3(body) |
| cg26917745 | 30939723 | | | |
| cg00063006 | 32847530 | − | Island | |
| cg11948766 | 49494499 | − | | |
| cg16167422 | 1448570 | − | S_Shore | |
| cg05034195 | 1.3E+08 | + | Island | |
| cg14042396 | 16153454 | − | N_Shore | |
| cg22517356 | 1361216 | + | N_Shore | TMEM88B(tss1500) |
| cg16517358 | 46164796 | − | Island | |
| cg17200137 | 48416896 | + | S_Shelf | GDF2(tss200) |
| cg13468625 | 1.4E+08 | − | S_Shelf | EXD3(body) |
| cg01807241 | 3043527 | − | Island | |
| cg18432441 | 8106632 | + | S_Shelf | TUB(body) |
| cg12561474 | 47590237 | + | Island | LOC100288866(body); NGFR(body) |
| cg03503642 | 75912711 | − | N_Shore | COL12A1(body) |
| cg03507824 | 64176993 | − | | |
| cg03352173 | 1.12E+08 | + | | SLC9C1(tss200) |
| cg21062780 | 887772 | − | Island | |
| cg08709360 | 1003376 | − | Island | |
| cg07031872 | 92788417 | + | N_Shore | SLC24A4(tss1500) |
| cg13304350 | 43084426 | + | | |
| cg19319928 | 1.17E+08 | − | | |
| cg24860562 | 31516481 | − | | LOC283710(body) |
| cg01215511 | 2975552 | − | | FLYWCH1(body) |
| cg01713438 | 1.75E+08 | − | | TNN(body) |
| cg00931742 | 57027301 | + | S_Shore | MOS(tss1500) |
| cg23887948 | 74494854 | + | | STRA6(body) |
| cg16244664 | 1.31E+08 | + | N_Shore | PIWIL1(tss1500) |
| cg12180613 | 1.32E+08 | − | Island | SHROOM1(body) |
| cg22190721 | 68269295 | + | Island | ESRP2(body) |
| cg25017403 | 31516404 | + | | LOC283710(body) |
| cg14206172 | 39466757 | + | S_Shore | FBXO17(tss1500) |
| cg20347666 | 1.38E+08 | + | | DGKI(body) |
| cg05231970 | 1.02E+08 | − | N_Shore | |
| cg10973146 | 1.04E+08 | + | Island | EXOC3L4(body) |
| cg00653460 | 1.01E+08 | + | S_Shore | RPL24(tss1500) |
| cg27263741 | 43099365 | − | | LINC00111(tss200) |
| cg19763471 | 48698761 | − | | CACNA1G(body) |
| cg09188304 | 84852691 | + | N_Shore | CRISPLD2(tss1500) |
| cg16725948 | 90167635 | + | N_Shore | |
| cg03324464 | 1.16E+08 | − | | |
| cg01841471 | 20879896 | − | S_Shelf | |
| cg07769957 | 46443657 | − | N_Shore | NOVA2(body) |
| cg02104434 | 2.48E+08 | − | Island | GCSAML(body); GCSAML- |
| | | | | AS1(tss1500); OR2C3(body) |
| cg21606036 | 78985900 | + | | CMYA5(body) |
| cg08732750 | 87598351 | + | Island | LOC339524(body) |
| cg07017454 | 92011979 | − | Island | |
| cg06776201 | 73146496 | − | | DPF3(body) |
| cg14264194 | 2.12E+08 | − | Island | RD3(body) |
| cg02704502 | 2036580 | − | S_Shelf | |
| cg16519668 | 75279566 | − | | BCAR1(body) |
| cg03222066 | 1.19E+08 | − | N_Shore | ABCG4(tss1500) |
| cg23270113 | 45417587 | + | | APOC1(tss1500) |
| cg08670906 | 1604081 | + | N_Shore | |
| cg10955566 | 1003126 | − | Island | |
| cg03554573 | 1.11E+08 | + | | C11orf53(body) |
| cg11052143 | 2.02E+08 | − | S_Shore | ALS2CR11(tss200) |
| cg26935536 | 77145356 | − | N_Shore | |
| cg20344448 | 14372431 | − | | FRMD4A(body) |
| cg11743829 | 1.46E+08 | − | Island | CD160(body); LOC100288142(body); |
| | | | | NBPF10(body) |
| cg27427581 | 8075716 | + | N_Shore | |
| cg15445037 | 60348161 | + | Island | CDH4(body) |
| cg24490133 | 2.46E+08 | − | | KIF26B(body) |
| cg17445155 | 76807543 | + | | PPEF2(body) |
| cg08158233 | 37351056 | − | | |
| cg01471372 | 38592365 | − | | TACC1(body) |
| cg18507732 | 65345681 | − | N_Shore | |
| cg23698536 | 1.24E+08 | − | N_Shore | FAM83A(body) |
| cg23012310 | 1.02E+08 | + | N_Shore | |
| cg21201393 | 38445522 | − | | EGFLAM(body); EGFLAM(tss200) |
| cg12443604 | 72357992 | − | S_Shore | BTBD17(tss200) |
| cg19273694 | 24233923 | − | S_Shore | MFSD2B(body) |
| cg15903282 | 1.35E+08 | + | N_Shore | EPHB1(tss1500) |
| cg15804973 | 1.37E+08 | + | S_Shore | MAP3K5(tss1500) |
| cg07064495 | 29304111 | − | Island | |
| cg17210938 | 59043173 | − | Island | TACSTD2(tss200) |
| cg05796704 | 11714218 | + | Island | FBXO2(body); FBXO44(tss1500) |
| cg17103155 | 4840199 | − | | |
| cg05139523 | 43435091 | − | N_Shelf | RIMS4(body) |
| cg15823502 | 41650768 | + | Island | |
| cg00138926 | 11118414 | + | | |
| cg05241134 | 1994419 | + | | MYOM2(body) |
| cg17397004 | 38863644 | − | N_Shore | KDELR3(tss1500) |
| cg25122629 | 54936822 | − | | |
| cg22414192 | 49621088 | + | Island | KCNG1(body) |
| cg24317190 | 54900936 | − | Island | |
| cg23255283 | 74240582 | + | | LINC00908(tss200) |
| cg25313468 | 57773308 | − | N_Shore | REST(tss1500) |
| cg14181528 | 19393870 | + | | |
| cg20038187 | 1.34E+08 | + | | |
| cg11936341 | 1604140 | − | N_Shore | |
| cg04098547 | 48125057 | + | | ABCC12(body) |
| cg11871991 | 83240213 | − | | CPEB1(body) |
| cg24413781 | 8273693 | − | Island | CERS4(tss1500) |
| cg23894948 | 21387055 | − | S_Shore | SLC7A4(tss1500) |
| cg00232387 | 2717526 | − | Island | DIRAS1(body) |
| cg12099459 | 1.48E+08 | + | S_Shore | NBPF8(body) |
| cg06502392 | 32634341 | + | | |
| cg26027576 | 27441373 | + | | |
| cg14299480 | 38876666 | − | Island | GGN(body) |
| cg17371020 | 1.54E+08 | − | | TRIM2(body); TRIM2(tss200) |
| cg10153349 | 13983893 | + | N_Shore | SPON1(tss1500) |
| cg05404792 | 56650847 | + | N_Shore | MT1L(tss1500) |
| cg12103951 | 88162314 | − | S_Shore | |
| cg24750156 | 58025315 | + | N_Shore | B4GALNT1(body) |
| cg14576824 | 2.13E+08 | − | N_Shore | RPS6KC1(tss200) |
| cg13251842 | 46508469 | + | | MIR4763(tss1500); MIRLET7A3(tss200); |
| | | | | MIRLET7B(tss1500); MIRLET7BHG |
| | | | | (body) |
| cg19628934 | 77499391 | + | Island | |
| cg19154323 | 2.09E+08 | − | S_Shore | PLEKHM3(tss1500) |
| cg15392457 | 1.2E+08 | − | S_Shore | |
| cg03694403 | 1.02E+08 | + | | |
| cg05204532 | 8597177 | − | Island | |
| cg15600051 | 2341440 | − | Island | ZFYVE28(body) |
| cg01711322 | 2.01E+08 | + | N_Shore | GPR25(body) |
| cg09953520 | 25812278 | + | | EBF2(body) |
| cg11705954 | 92011732 | + | N_Shore | |
| cg26665274 | 6747409 | − | N_Shore | BMP2(tss1500) |
| cg09546802 | 1.45E+08 | − | S_Shore | PRELID2(tss1500) |
| cg24377495 | 6660075 | − | Island | |
| cg06181761 | 92042045 | + | S_Shore | |
| cg20794351 | 21908746 | + | S_Shelf | |
| cg17149030 | 99549047 | − | | PGPEP1L(body); PGPEP1L(tss200) |
| cg23829948 | 3288458 | + | | |
| cg18623649 | 1.25E+08 | + | | |
| cg05156120 | 41723125 | − | N_Shore | MEOX1(body) |
| cg02630888 | 15657820 | + | S_Shore | FBXL5(tss1500) |
| cg09320662 | 1.33E+08 | + | S_Shore | LRCOL1(body) |
| cg01973920 | 980997 | − | | |
| cg12585923 | 42756876 | − | | CCDC152(tss200) |
| cg00567703 | 54413101 | + | Island | HOXC4(body); HOXC5(body); HOXC6 |
| | | | | (body) |
| cg12589798 | 44839734 | − | S_Shore | CDH22(body) |
| cg09269945 | 55837692 | − | N_Shore | BMP7(body) |
| cg21450652 | 46875436 | − | Island | PRSS42(body) |
| cg17142470 | 22409684 | + | S_Shore | SORBS3(body) |
| cg21207436 | 74815316 | + | | VRTN(body) |
| cg17205324 | 23835595 | + | Island | EFS(tss1500) |
| cg02470697 | 48844916 | + | Island | GTF2A1L(tss200); STON1- |
| | | | | GTF2A1L(body) |
| cg11693285 | 1.32E+08 | − | Island | |
| cg17780447 | 22700869 | − | Island | |
| cg08043020 | 8038083 | − | N_Shelf | |
| cg07618085 | 77924582 | − | S_Shore | TBC1D16(body) |
| cg26023303 | 1.04E+08 | + | N_Shore | |
| cg14166701 | 1.05E+08 | − | | ASPG(body) |
| cg15255946 | 32729563 | + | Island | HLA-DQB2(body) |
| cg27423692 | 1.35E+08 | − | S_Shore | |
| cg22910549 | 50985117 | + | Island | KLHDC7B(tss1500) |
| cg25718438 | 43743921 | + | | WFDC5(tss200) |
| cg16003238 | 65670876 | − | S_Shore | IGDCC3(tss1500) |
| cg22268510 | 32118420 | − | Island | PRRT1(body) |
| cg02834447 | 2.38E+08 | − | | |
| Cg10249734 | 80292079 | − | Island | SECTM1(tss200) |
| cg01717649 | 66821665 | − | N_Shelf | |
| cg04612959 | 4183976 | + | | SDK1(body) |
| cg18414021 | 1E+08 | − | Island | CCDC85C(body) |
| cg21697667 | 1.21E+08 | + | | |
| cg19042287 | 1.08E+08 | − | S_Shore | NR2E1(body) |
| cg25573884 | 25992424 | + | S_Shore | |
| cg10081626 | 1.03E+08 | + | N_Shore | |
| cg27478242 | 54611928 | + | Island | |
| cg08846011 | 1.17E+08 | − | | |
| cg00987015 | 62688751 | + | N_Shelf | CHRM1(body) |
| cg13928759 | 95517382 | + | | LGI1(tss200) |
| cg14328523 | 35036350 | − | | |
| cg26451735 | 74494900 | + | | STRA6(body) |
| cg01355876 | 1.31E+08 | + | Island | LOC728637(tss200) |
| cg10772086 | 1.33E+08 | − | N_Shore | TF(tss1500) |
| cg19706390 | 10709702 | + | S_Shore | CASZ1(body) |
| cg26758396 | 6104274 | + | S_Shore | FERMT1(tss200) |
| cg13326914 | 52862092 | + | | LRRC66(body) |
| cg01054354 | 1E+08 | − | N_Shore | LYSMD4(body) |
| cg02389040 | 31650786 | − | Island | |
| cg14924160 | 41496235 | − | N_Shore | SUGT1P3(tss1500); TPTE2P5(tss1500) |
| cg10668414 | 34895979 | − | S_Shore | |
| cg11786623 | 1.63E+08 | + | | |
| cg21122199 | 58499720 | + | S_Shore | C17orf64(tss200) |
| cg21401642 | 1.74E+08 | + | N_Shore | |
| cg02841651 | 1.33E+08 | + | | |
| cg09357462 | 1.12E+08 | + | S_Shore | DUSP5(body) |
| cg04466022 | 37618123 | + | Island | MDGA1(body) |
| cg20770435 | 35105154 | + | S_Shore | CELF4(body) |
| cg10372829 | 20751257 | − | Island | |
| cg07634645 | 14720938 | − | Island | PLBD1(tss200) |
| cg01423389 | 1.71E+08 | − | S_Shore | |
| cg12288726 | 1.27E+08 | − | Island | ARF5(tss1500) |
| cg16654603 | 46755422 | + | N_Shore | |
| cg01065977 | 18549689 | + | S_Shore | ISYNA1(tss1500) |
| cg27560452 | 49499385 | − | Island | |
| cg22570042 | 20799532 | + | Island | CCDC144NL(tss200); LOC440416(body) |
| cg27315996 | 21925182 | − | S_Shore | DMTN(body) |
| cg15656462 | 1.35E+08 | + | | |
| cg20167366 | 30094980 | − | N_Shore | |
| cg17178468 | 43049442 | + | S_Shore | |
| cg05349837 | 54430991 | + | S_Shelf | HOXC4(body) |
| cg07044859 | 1.01E+08 | − | Island | |
| cg13521077 | 81260418 | − | S_Shelf | C4orf22(body) |
| cg16619576 | 45398036 | + | Island | |
| cg08317694 | 78632144 | − | N_Shore | CRABP1(tss1500) |
| cg04661001 | 19217217 | − | N_Shelf | SLC25A42(body) |
| cg25283465 | 1.11E+08 | + | Island | |
| cg03483626 | 1.11E+08 | − | S_Shore | KCNA3(tss1500) |
| cg16448636 | 21209509 | + | | |
| cg14044120 | 1.4E+08 | + | S_Shore | ENTPD2(tss1500) |
| cg12343363 | 1.03E+08 | + | | METTL21C(body) |
| cg19413350 | 57351067 | − | Island | |
| cg03257743 | 11123616 | − | | |
| cg15750303 | 18765848 | − | Island | NTSC1B(body); NTSC18- |
| | | | | RDH14(body) |
| cg08439880 | 1.34E+08 | + | N_Shore | SRPRB(tss1500) |
| cg17160439 | 64815744 | + | S_Shore | NAALADL1(body) |
| cg06908857 | 1353627 | + | | |
| cg01937803 | 1.04E+08 | + | Island | |
| cg21511948 | 37820236 | − | N_Shelf | ELFN2(body) |
| cg06355129 | 1.57E+08 | + | Island | NES(body) |
| cg24973864 | 28810645 | + | S_Shore | |
| cg11515089 | 18553533 | − | | |
| cg08837626 | 36011406 | + | Island | |
| cg00797104 | 32847605 | − | Island | |
| cg05096209 | 28829433 | + | N_Shelf | LOC401242(body) |
| cg13791841 | 71954381 | − | N_Shore | PHOX2A(body) |
| cg08048268 | 1.34E+08 | + | Island | SRPRB(tss200) |
| cg14482569 | 1.3E+08 | + | S_Shore | ZBTB44(tss1500) |
| cg25788719 | 3713002 | − | N_Shore | HSPA12B(tss1500) |
| cg27498545 | 8717323 | − | | SOGA2(tss200) |
| cg22020227 | 1.15E+08 | − | | OLFML3(tss1500) |
| cg01795955 | 2E+08 | + | Island | |
| cg16906964 | 4660171 | + | | |
| cg16751357 | 35150268 | − | Island | |
| cg19218505 | 8102016 | − | N_Shore | TUB(body); TUB(tss1500) |
| cg19442201 | 40572550 | + | Island | |
| cg16760981 | 63688531 | − | S_Shelf | |
| cg13392957 | 1.48E+08 | − | Island | NBPF8(body) |
| cg05399330 | 55464530 | − | S_Shore | BSND(tss200) |
| cg22196260 | 50936172 | − | S_Shore | MYBPC2(body) |
| cg09235562 | 384251 | + | Island | |
| cg10112270 | 21913458 | − | | |
| cg13953753 | 1.19E+08 | + | Island | VAX1(body) |
| cg16861209 | 33361127 | − | | SLC7A9(tss1500) |
| cg10600889 | 37123767 | + | | FBXO47(tss200) |
| cg04178634 | 2525231 | + | Island | GNG7(body) |
| cg26187194 | 1.32E+08 | − | S_Shore | |
| cg05958582 | 1.02E+08 | + | | PKD2L1(tss200) |
| cg11372818 | 1.32E+08 | + | S_Shore | |
| cg13810723 | 1.2E+08 | + | S_Shore | SRRM4(body) |
| cg14264372 | 6248114 | + | S_Shore | |
| cg17471939 | 3600879 | − | | TP73(body) |
| cg03923640 | 87174624 | + | | |
| cg14479947 | 40544427 | + | Island | C15orf56(body); PAK6(body); PAK6(tss1500) |
| cg00166722 | 10149974 | − | Island | FANCD2OS(tss200) |
| cg15594585 | 2.28E+08 | − | N_Shore | |
| cg04190888 | 49879914 | + | | |
| cg17558772 | 1406218 | + | Island | ADARB2(body) |
| cg26510023 | 45694889 | + | Island | |
| cg23109891 | 38379218 | + | Island | SOX10(body) |
| cg10674793 | 1.84E+08 | − | N_Shore | DVL3(tss1500) |
| cg26135325 | 1.53E+08 | − | | LCE3A(body) |
| cg14503395 | 1.26E+08 | − | Island | CRB2(body) |
| cg27140633 | 63685905 | + | Island | |
| cg12872238 | 1.11E+08 | + | S_Shore | TRPC5(tss1500); ZCCHC16(body) |
| cg24837256 | 19284087 | − | S_Shore | IFFO2(tss1500) |
| cg06066088 | 3600383 | − | S_Shore | P2RX5(tss1500); P2RX5- |
| | | | | TAX1BP3(tss1500) |
| cg12528597 | 57232556 | + | Island | RTN4RL2(body) |
| cg22708150 | 31649619 | + | N_Shore | LY6G5C(tss1500) |
| cg23374892 | 51165845 | − | S_Shore | SHANK1(body) |
| cg19214300 | 56709503 | + | N_Shore | MT1IP(tss1500) |
| cg10571901 | 43295303 | − | Island | |
| cg16430538 | 10459836 | + | S_Shore | ZNF518B(tss1500) |
| cg16810031 | 38024146 | + | Island | ZPBP2(tss1500) |
| cg27046335 | 35198290 | + | | PRLR(body) |
| cg06399427 | 1.62E+08 | + | | |
| cg18795569 | 3480508 | + | N_Shore | C19orf77(body) |
| cg14654731 | 1.23E+08 | − | N_Shore | PP12613(tss1500); TMEM155(body) |
| cg04662609 | 1.81E+08 | − | Island | |
| cg00847453 | 1.11E+08 | + | N_Shore | DDO(body) |
| cg13148669 | 23003692 | + | Island | |
| cg13986670 | 11708792 | + | Island | FBXO2(body) |
| cg10521948 | 8751524 | − | Island | |
| cg06569542 | 98946673 | + | S_Shore | ARHGAP19- |
| | | | | SLIT1(body); SLIT1(tss1500) |
| cg27434086 | 41218082 | + | N_Shore | APBB2(tss1500) |
| cg08883853 | 1026168 | + | Island | CYP2W1(body) |
| cg26700447 | 6292511 | − | Island | CCKBR(body) |
| cg04295144 | 10407184 | − | Island | ICAM5(body) |
| cg14417784 | 1.31E+08 | + | Island | |
| cg19234983 | 2.12E+08 | + | N_Shore | |
| cg08979515 | 1.12E+08 | − | N_Shore | ABHD10(tss200) |
| cg22697108 | 1.71E+08 | − | | |
| cg03476791 | 99067162 | − | N_Shelf | |
| cg01882498 | 83934956 | − | S_Shore | LIN54(tss1500) |
| cg17349199 | 1.18E+08 | + | Island | C10orf82(body) |
| cg13947534 | 1.35E+08 | − | Island | C9orf171(body) |
| cg12924095 | 1.51E+08 | + | Island | G38P1(tss1500); LOC100652758(body) |
| cg14501281 | 10360265 | − | | |
| cg14794655 | 54558352 | − | S_Shore | C2orf73(body) |
| cg11510586 | 72027409 | − | Island | |
| cg19547370 | 1.43E+08 | − | S_Shore | PCOLCE2(tss1500) |
| cg06988349 | 69991859 | + | S_Shore | ATOH7(body) |
| cg22486834 | 52859107 | − | N_Shore | GSTA4(body) |
| cg15037663 | 24797835 | − | S_Shore | DFNA5(tss1500); DFNA5(tss200) |
| cg20731937 | 48336164 | − | | |
| cg11246233 | 61028854 | − | | |
| cg02799090 | 1.31E+08 | + | N_Shore | |
| cg20851828 | 28601440 | − | N_Shore | |
| cg22743002 | 34123215 | + | S_Shore | GRM4(body) |
| cg07275979 | 1.24E+08 | + | N_Shore | TBC1D31(tss1500) |
| cg02980055 | 47866183 | + | N_Shore | |
| cg05764839 | 32118298 | − | Island | PRRT1(body) |
| cg05096637 | 1.22E+08 | + | | |
| cg24113950 | 36577900 | − | S_Shelf | |
| cg02948944 | 26242737 | − | | |
| cg14153919 | 78631963 | + | N_Shore | CRABP1(tss1500) |
| cg26288449 | 56048453 | + | S_Shore | SBK2(tss200) |
| cg05212464 | 1.54E+08 | + | S_Shore | |
| cg09755594 | 85527430 | + | | WDR63(tss1500) |
| cg18147098 | 2.13E+08 | + | S_Shore | |
| cg18530324 | 46064992 | − | N_Shore | CTIF(tss1500) |
| cg01287088 | 1.77E+08 | + | Island | PFN3(body) |
| cg02850715 | 1.3E+08 | − | N_Shore | ST14(tss1500) |
| cg19463256 | 46915351 | + | Island | CCDC8(body) |
| cg24927974 | 35078269 | − | S_Shore | DPY19L1(tss1500) |
| cg08474748 | 74350214 | + | Island | |
| cg23848052 | 66318701 | − | | MEGF11(body) |
| cg22383874 | 48670670 | + | | CACNA1G(body) |
| cg23595451 | 1035462 | + | | |
| cg06462065 | 55885168 | − | N_Shelf | |
| cg10356759 | 248889 | − | S_Shore | IQSEC3(body); LOC574538(body) |
| cg19530728 | 45279132 | − | Island | BTBD19(body) |
| cg22814764 | 1.27E+08 | + | | C3orf56(tss200) |
| cg22775123 | 39761595 | + | S_Shore | DAAM2(body) |
| cg23484555 | 62516670 | − | Island | |
| cg14348540 | 22224272 | + | N_Shore | SLC39A14(tss1500) |
| cg11041817 | 46685327 | − | Island | HQXB7(body) |
| cg19766591 | 2978150 | + | | |
| cg16855352 | 761284 | + | | |
| cg15745284 | 32064923 | + | Island | TNXB(body) |
| cg12884551 | 1673208 | − | | |
| cg06834313 | 1.09E+08 | − | | |
| cg23259375 | 26016732 | − | N_Shelf | |
| cg11132536 | 74711944 | − | S_Shelf | VSX2(body) |
| cg21443699 | 1.43E+08 | + | | C7orf34(body) |
| cg10694914 | 1.11E+08 | + | | POU2AF1(tss1500) |
| cg05816193 | 26018127 | + | N_Shelf | HIST1H1A(tss200) |
| cg19065106 | 53722177 | − | S_Shelf | SP7(body) |
| cg24612707 | 1.28E+08 | − | N_Shore | HILPDA(tss1500) |
| cg05545777 | 1.01E+08 | + | | |
| cg15536947 | 65471303 | − | | PITPNC1(body) |
| cg10494684 | 26645200 | + | N_Shore | TMEM97(tss1500) |
| cg26004810 | 70048845 | − | N_Shore | FADD(tss1500) |
| cg21862126 | 1076296 | − | S_Shelf | LOC254099(body) |
| cg26385222 | 1.5E−08 | + | Island | TMEM176A(tss1500); TMEM176B |
| | | | | (body) |
| cg16276523 | 1.61E+08 | + | Island | PVRL4(body) |
| cg17527422 | 48579245 | + | S_Shore | C12orf68(body) |
| cg25251478 | 30853959 | − | S_Shore | DDR1(body) |
| cg09334514 | 2.37E+08 | + | N_Shore | LGALS8(body); LGALS8(tss1500); LGALS8 |
| | | | | (tss200); LGALS8-AS1(body) |
| cg04872399 | 2.01E+08 | − | | TNNT2(body) |
| cg03330558 | 31516127 | − | | LOC283710(body) |
| cg21474062 | 1.41E+08 | − | | ZBTB38(body) |
| cg10612096 | 2.32E+08 | + | | SPATA3(body) |
| cg16379671 | 1.32E+08 | − | Island | |
| cg09373597 | 1.39E+08 | + | N_Shore | C3orf72(body) |
| cg15205559 | 1.89E+08 | + | | |
| cg19618897 | 65242892 | − | S_Shore | |
| cg13981054 | 18020115 | + | | |
| cg15326579 | 1.32E+08 | − | | |
| cg19284039 | 2292895 | + | Island | ASCL2(tss1500) |
| cg18500300 | 45128199 | + | Island | NACAD(body) |
| cg00974095 | 40154614 | + | N_Shelf | HPCAL4(body) |
| cg08139206 | 1.04E+08 | − | S_Shelf | |
| cg23202468 | 42705828 | + | | ZBTB47(body) |
| cg19276110 | 20396593 | − | | PLA2G5(tss200) |
| cg22691119 | 68881372 | − | | |
| cg07070113 | 79283738 | − | N_Shore | LINC00482(tss1500) |
| cg11672277 | 32556104 | − | N_Shore | MAPRE2(tss1500) |
| cg27176129 | 43937155 | + | S_Shore | MATN4(body); MATN4(tss1500); MATN4 |
| | | | | (tss200); RBPJL(body) |
| cg05504117 | 71898904 | − | S_Shore | |
| cg00055529 | 37123835 | + | | FBXO47(tss200) |
| cg09100271 | 48675911 | − | N_Shore | |
| cg20408181 | 2.09E+08 | + | Island | |
| cg07830534 | 38953285 | − | Island | KRT28(body) |
| cg01773685 | 51330453 | − | Island | KLK15(body) |
| cg26383138 | 2119533 | − | | MYT1L(body) |
| cg15925365 | 52858998 | + | N_Shore | GSTA4(body) |
| cg10196558 | 1.55E+08 | − | Island | |
| cg04685442 | 49776492 | + | | |
| cg09476130 | 1.6E+08 | + | Island | CCDC19(tss200) |
| cg10321236 | 76849101 | − | Island | MYO7A(body) |
| cg16723488 | 21266947 | − | Island | APOB(tss200) |
| cg00582337 | 40274811 | + | Island | HSPB9(body); KAT2A(tss1500) |
| cg12065616 | 94954828 | + | | MIR5700(tss1500) |
| cg03313542 | 1.3E+08 | − | S_Shore | TMEM209(tss1500) |
| cg25433316 | 10197543 | − | | CYS1(body) |
| cg16135695 | 1889970 | − | | |
| cg08363339 | 57618487 | − | S_Shore | SLMO2(tss1500); SLMO2- |
| | | | | ATP5E(tss1500) |
| cg10612274 | 63827432 | − | | MACROD1(body) |
| cg06919203 | 3644151 | − | S_Shelf | GFRA4(tss200) |
| cg04130572 | 42806000 | + | | JPH2(body) |
| cg25673584 | 46417551 | − | Island | NANOS2(body) |
| cg00363082 | 1.73E+08 | − | N_Shore | |
| cg17866778 | 26233442 | + | | |
| cg20850873 | 10149803 | − | Island | FANCD2OS(body) |
| cg18316498 | 1.11E+08 | − | | POU2AF1(tss200) |
| cg11166287 | 1.05E+08 | + | | |
| cg25598308 | 1.2E+08 | + | | GRIK4(body) |
| cg17921412 | 5495287 | − | Island | |
| cg16037711 | 59280841 | + | N_Shore | LOC100131060(body) |
| cg08976101 | 44328423 | − | S_Shore | ALX4(body) |
| cg02329835 | 98315369 | − | Island | |
| cg15070513 | 37782685 | + | N_Shore | PPP1R1B(tss1500) |
| cg14789818 | 2.28E+08 | − | Island | |
| cg00807235 | 4183883 | − | | |
| cg02276070 | 1.5E+08 | + | S_Shore | ARSI(tss1500) |
| cg22992730 | 4784940 | + | N_Shore | |
| cg11660018 | 86510915 | + | N_Shore | PRSS23(tss1500) |
| cg09077319 | 69285899 | − | Island | |
| cg09467436 | 72079424 | + | Island | |
| cg11287647 | 12725352 | + | N_Shore | ARL4A(tss1500) |
| cg14382976 | 1.17E+08 | − | Island | ASZ1(tss200) |
| cg20772512 | 38022359 | − | N_Shore | DNALI1(tss200) |
| cg01209984 | 47469679 | + | | |
| cg02623200 | 1.19E+08 | − | N_Shore | SPAG17(body) |
| cg08774009 | 49309094 | + | Island | |
| cg02438164 | 4651732 | + | | |
| cg09141303 | 1.04E+08 | − | S_Shore | SLC9B2(tss200) |
| cg24996985 | 1.07E+08 | − | | |
| cg00435606 | 16257223 | + | S_Shore | CENPV(tss1500) |
| cg06239923 | 55923774 | + | N_Shelf | MIR5095(body) |
| cg26621607 | 1.33E+08 | + | Island | LRCOL1(body) |
| cg13846270 | 51417929 | + | Island | DLEU7(tss200); DLEU7-AS1(body) |
| cg06974662 | 8277080 | + | N_Shore | |
| cg26107597 | 31343283 | − | N_Shore | COCH(tss1500) |
| cg09618778 | 66838934 | + | Island | RHOD(body) |
| cg13891539 | 30322730 | + | N_Shore | |
| cg00583535 | 2.01E+08 | + | | IGFN1(tss200) |
| cg05506600 | 72447967 | + | | |
| cg21708738 | 42175351 | + | | C2orf91(body) |
| cg02830202 | 86953383 | − | | |
| cg08352755 | 1.46E+08 | + | Island | |
| cg15056966 | 1.2E+08 | + | N_Shore | |
| cg24720336 | 1.51E+08 | + | N_Shore | PIP5K1A(tss1500) |
| cg03365937 | 45688659 | − | S_Shore | CHST1(tss1500) |
| cg16057826 | 36041334 | + | Island | CLIC6(tss1500) |
| cg23587980 | 52595568 | + | N_Shore | KRT86(tss200) |
| cg03242265 | 1.48E+08 | + | Island | NBPF8(body) |
| cg06532268 | 25732817 | − | Island | HIST1H2APS1(body) |
| cg19987219 | 47016749 | + | | KNCN(body); MKNK1-AS1(body) |
| cg17121140 | 1.12E+08 | − | N_Shore | |
| cg26055950 | 3600852 | − | | TP73(body) |
| cg03555157 | 1.28E+08 | + | | LIMS2(body) |
| cg14329783 | 75779857 | + | Island | |
| cg03400335 | 1.22E+08 | − | | ILDR1(tss1500) |
| cg09359103 | 1.55E+08 | + | | KCNN3(body) |
| cg18041277 | 57030824 | + | Island | |
| cg17518825 | 46523461 | − | N_Shore | EPAS1(tss1500) |
| cg07508649 | 31873772 | + | S_Shelf | C2(body) |
| cg13623268 | 78912437 | + | | PCSK5(body) |
| cg10671830 | 7652103 | + | | SORCS2(body) |
| cg06889571 | 1416791 | − | S_Shore | ADARB2(body) |
| cg10123421 | 11785052 | + | Island | ZNF833P(body) |
| cg11068289 | 78955884 | + | S_Shore | |
| cg25821249 | 35423464 | − | | |
| cg08601628 | 10555466 | − | | C8orf74(body) |
| cg17352045 | 32847702 | − | Island | |
| cg18753928 | 1.13E+08 | + | S_Shore | SPICE1(tss1500) |
| cg08373187 | 1.35E+08 | + | N_Shelf | CXCL14(body) |
| cg16111190 | 30860887 | − | | DDR1(body) |
| cg16500916 | 54228169 | + | N_Shore | |
| cg15089314 | 78365255 | − | N_Shore | BHMT2(tss1500); DMGDH(body) |
| cg22167400 | 26633978 | + | Island | KRT18P55(body) |
| cg08245096 | 1.4E+08 | − | N_Shore | |
| cg08685733 | 42215609 | + | N_Shelf | |
| cg06724402 | 1.02E+08 | + | N_Shelf | ANGPTL5(body) |
| cg15209566 | 11784774 | − | N_Shore | ZNF833P(tss200) |
| cg06084073 | 40544353 | − | Island | C15orf56(body); PAK6(body); PAK6 |
| | | | | (tss1500) |
| cg12717963 | 1.81E+08 | − | Island | TRIM7(body) |
| cg01431140 | 35118924 | + | | |
| cg23053961 | 4300432 | + | S_Shore | GLIS3(tss1500) |
| cg04391540 | 48048129 | − | Island | ZNF541(body) |
| cg07207043 | 7051497 | − | Island | |
| cg18938392 | 1.57E+08 | − | | |
| cg17436656 | 53627106 | + | S_Shore | RARG(tss1500) |
| cg05645982 | 1.24E+08 | + | N_Shore | FAM83A(body) |
| cg04299200 | 534332 | + | N_Shore | |
| cg24068229 | 39523395 | + | S_Shore | FBXO27(tss200) |
| cg04573398 | 86086005 | + | Island | CCDC81(body) |
| cg16791134 | 12200856 | + | S_Shore | SYN2(body); TIMP4(tss200) |
| cg13191951 | 71685353 | − | | THSD4(body) |
| cg15235893 | 2.35E+08 | + | Island | ARL4C(tss1500) |
| cg01227744 | 20644371 | + | N_Shelf | |
| cg12582028 | 1.52E+08 | − | S_Shore | MRPL9(tss1500); OAZ3(body) |
| cg03162823 | 95941571 | − | N_Shore | USP44(body) |
| cg26280727 | 49000998 | + | Island | LMTK3(body) |
| cg20075675 | 1.06E+08 | − | N_Shelf | NUAK1(body) |
| cg18323095 | 62515771 | − | N_Shore | |
| cg12301695 | 1.66E+08 | + | | SLC38A11(tss200) |
| cg17321385 | 1.32E+08 | + | N_Shore | |
| cg23397023 | 1.49E+08 | − | S_Shore | |
| cg05407338 | 8213522 | + | | ARHGEF15(tss1500); ARHGEF15(tss200) |
| cg26934993 | 40274722 | − | Island | HSP89(tss200); KAT2A(tss1500) |
| cg21211253 | 1.39E+08 | + | S_Shore | |
| cg04319873 | 1.64E+08 | − | S_Shore | |
| cg14466942 | 17282700 | + | S_Shore | RBM24(body); RBM24(tss1500) |
| cg13595308 | 1.29E+08 | − | | |
| cg02608963 | 46878553 | + | Island | |
| cg09031776 | 1.18E−08 | − | | |
| cg17052552 | 41654404 | + | N_Shore | ANK1(body) |
| cg23629935 | 90167459 | + | N_Shore | |
| cg12244617 | 50737978 | − | Island | PLXNB2(body) |
| cg19916129 | 1.13E+08 | − | S_Shore | TUBGCP3(tss1500) |
| cg24735235 | 16163479 | − | N_Shore | FLJ37453(body) |
| cg15442037 | 69286293 | + | Island | |
| cg09384937 | 49359996 | + | N_Shelf | WNT10B(body) |
| cg00965168 | 2.28E+08 | − | Island | |
| cg17878899 | 1.18E+08 | − | Island | KIAA1210(tss200) |
| cg22904173 | 1.2E+08 | + | | |
| cg00202454 | 42371886 | + | S_Shore | PLA2G4D(body) |
| cg03759229 | 28601417 | − | N_Shore | |
| cg15772157 | 22766320 | − | Island | |
| cg16602316 | 93698053 | − | | |
| cg04598224 | 11784514 | + | N_Shore | ZNF833P(tss1500) |
| cg08107402 | 35069269 | − | S_Shore | SCGB1B2P(tss1500) |
| cg24702147 | 7071814 | − | | MIR141(tss1500); MIR200C(tss1500) |
| cg00509207 | 31078979 | − | N_Shore | TSPAN11(tss1500) |
| cg18493761 | 1.25E+08 | − | | |
| cg12564567 | 1.16E+08 | + | Island | |
| cg20307496 | 37955927 | − | N_Shore | CDC42EP1(tss1500) |
| cg15244786 | 54446279 | + | N_Shore | HOXC4(body); HOXC4(tss1500) |
| cg10422709 | 53083978 | − | S_Shore | |
| cg16773028 | 1.11E−08 | − | N_Shore | KCNA2(body) |
| cg06889535 | 1.26E+08 | + | | KSRREL3(body) |
| cg03463990 | 74534692 | + | N_Shelf | CCDC33(body) |
| cg25162921 | 21801897 | − | N_Shore | MTAP(tss1500) |
| cg16149919 | 99548954 | + | | PGPEP1L(body); PGPEP1L(tss200) |
| cg23591302 | 42876090 | + | N_Shore | PRICKLE1(body) |
| cg08958251 | 27270390 | − | | |
| cg02136452 | 49872492 | + | Island | ZNF423(body) |
| cg27315077 | 46404584 | + | N_Shelf | |
| cg01310397 | 1.95E+0E | − | S_Shore | MUC4(body) |
| cg23507802 | 13914731 | + | | WNT7A(body) |
| cg16581017 | 1.01E+08 | − | | MYL10(body) |
| cg11507770 | 1.29E+08 | − | Island | |
| cg19728382 | 1.73E+08 | − | S_Shore | STC2(tss1500) |
| cg07160793 | 382090 | + | | |
| cg15489799 | 1098380 | − | Island | |
| cg00469015 | 69744684 | − | N_Shore | RPLP1(tss1500) |
| cg25325636 | 57766896 | + | Island | ZNF831(body) |
| cg06895640 | 93277191 | + | S_Shore | SMCO4(tss1500) |
| cg09893504 | 11273901 | + | | C2orf50(body) |
| cg01820273 | 35754906 | − | Island | CLPSL1(body) |
| cg05674602 | 16153506 | + | N_Shore | |
| cg11367633 | 40247916 | − | S_Shore | CHD6(tss1500) |
| cg22284422 | 74240476 | − | | LINC00908(tss200) |
| cg08896939 | 29297380 | + | N_Shore | RNF135(tss1500) |
| cg06809326 | 20799526 | + | Island | CCDC144NL(tss200); LOC440416(body) |
| cg27282900 | 30095258 | − | Island | |
| cg18263397 | 1.11E+08 | + | S_Shelf | TRPC5(tss1500); ZCCHC16(body) |
| cg12544557 | 39019545 | − | Island | KRT12(body) |
| cg18632631 | 7284049 | − | N_Shore | TNK1(tss1500) |
| cg12258454 | 1.26E+08 | − | N_Shore | |
| cg07747661 | 1.25E+08 | + | | PKNOX2(body) |
| cg01066472 | 75591029 | + | Island | |
| cg09606832 | 10405427 | + | S_Shore | TFAP2A(body) |
| cg06365016 | 1.31E+08 | − | N_Shore | PIWIL1(tss1500) |
| cg04994481 | 65345381 | + | N_Shore | |
| cg25607643 | 40730161 | − | S_Shore | |
| cg19380722 | 1.02E+08 | + | | PKD2L1(tss200) |
| cg17642369 | 47783644 | − | | |
| cg08334034 | 93615039 | − | N_Shore | RGMA(body) |
| cg05496603 | 44237813 | + | N_Shore | TMEM151B(tss1500) |
| cg11968118 | 16474839 | − | S_Shelf | |
| cg26906998 | 17108846 | + | Island | PLD6(body) |
| cg23021340 | 1.11E+08 | − | N_Shore | METTL24(body) |
| cg25620797 | 55921863 | − | S_Shore | PNPT1(tss1500) |
| cg17179314 | 78933820 | − | Island | CHRNB4(tss1500) |
| cg01111041 | 32121055 | + | N_Shore | LOC100507547(body); PPT2(tss1500); |
| | | | | PPT2(tss200); PPT2- |
| | | | | EGFL8(tss1500); PRRT1(tss1500) |
| cg20003893 | 1.44E+08 | + | Island | |
| cg12379307 | 1.23E+08 | − | N_Shore | SMPDL3A(tss1500) |
| cg26561986 | 95939442 | + | N_Shelf | USP44(body) |
| cg01700504 | 20805391 | + | N_Shore | GJB6(body); GJB6(tss200) |
| cg15520962 | 55888146 | − | N_Shore | TMEM190(tss200) |
| cg04059762 | 53544020 | − | | |
| cg17757980 | 46971495 | − | Island | SYT15(tss1500) |
| cg16570507 | 88024598 | + | S_Shore | GRID1(body); MIR346(tss200) |
| cg02445968 | 1449028 | − | | ADARB2(body) |
| cg00000029 | 53468112 | + | N_Shore | RBL2(tss1500) |
| cg01984511 | 1.13E+08 | + | S_Shore | DTX1(body) |
| cg14435498 | 1.33E+08 | − | | |
| cg17987384 | 26634469 | + | Island | KRT18P55(tss200) |
| cg04100190 | 40783264 | + | S_Shore | COL9A2(tss1500) |
| cg15467577 | 35547085 | − | N_Shelf | HPN(body) |
| cg25521622 | 2.32E+08 | + | S_Shore | |
| cg06760077 | 1.19E+08 | + | | |
| cg20655369 | 89924975 | − | S_Shelf | LINC00925(body) |
| cg02227331 | 2.35E+08 | − | Island | LOC100286922(body); UGT1A10(body); |
| | | | | UGT1A3(body); UGT1A4(body); |
| | | | | UGT1A5(body); UGT1A6(body); UGT1A7(body); |
| | | | | UGT1A8(body); UGT1A9(body) |
| cg16194588 | 49002477 | + | Island | LMTK3(body) |
| cg17641566 | 73565966 | − | S_Shore | CDH23(body) |
| cg10817554 | 2.32E+08 | − | S_Shore | |
| cg20367961 | 41950237 | − | | EDN2(body) |
| cg22352176 | 2.23E+08 | + | N_Shore | DISP1(tss1500) |
| cg16179507 | 43295306 | − | Island | |
| cg20464732 | 1.11E+08 | − | | |
| cg10249506 | 42734458 | + | Island | |
| cg07785717 | 54485321 | + | Island | CACNG8(body); MIR935(tss1500) |
| cg12728621 | 34743092 | − | | EPB41L1(body) |
| cg23515411 | 1.35E+08 | − | Island | |
| cg23247955 | 48475522 | − | S_Shore | LRRC59(tss1500) |
| cg12281620 | 2525290 | + | Island | GNG7(body) |
| cg04495354 | 57294356 | + | | PLLP(body) |
| cg17510121 | 35182910 | − | S_Shore | SCUBE3(body) |
| cg13977320 | 1.17E+08 | + | N_Shore | KIF12(body) |
| cg03892457 | 1.59E+08 | − | N_Shore | CADM3(body) |
| cg23487312 | 62401106 | − | | CADPS(body) |
| cg01116161 | 28810402 | + | Island | |
| cg26133081 | 20736342 | − | S_Shore | GJA3(tss1500) |
| cg08577046 | 77893746 | + | | |
| cg21370924 | 1.41E+08 | + | | ZBTB38(body) |
| cg06708213 | 74864041 | − | N_Shore | MGAT58(tss1500) |
| cg13163521 | 41803428 | + | N_Shore | LTK(body) |
| cg13275609 | 1.2E+08 | − | | |
| cg17191443 | 43929629 | + | N_Shelf | MATN4(body) |
| cg03352287 | 1.52E+08 | + | Island | CRCT1(body) |
| cg10750464 | 11534062 | + | S_Shore | CCDC151(body) |
| cg06766541 | 1.29E+08 | − | Island | |
| cg07795548 | 69457652 | − | N_Shore | CYB5B(tss1500) |
| cg05766948 | 1.37E+08 | − | | |
| cg10658779 | 77871675 | + | N_Shore | C10orf11(body) |
| cg00255889 | 26586283 | − | Island | ATP8A2(body) |
| cg26584034 | 1.5E+08 | − | | SYNPO(body) |
| cg02455706 | 849188S1 | − | | CRISPLD2(body) |
| cg08121453 | 2.49E+08 | + | Island | |
| cg15927699 | 37856881 | + | | |
| cg05624199 | 41168318 | − | N_Shore | |
| cg17267654 | 1.7E+08 | + | | |
| cg20240715 | 35828503 | − | N_Shelf | KCNE1(body); KCNE1(tss1500) |
| cg08709333 | 73225760 | − | | |
| cg22440848 | 1.84E+08 | + | S_Shore | |
| cg20814179 | 940893 | − | Island | TMEM175(body) |
| cg16922753 | 1113291 | − | Island | |
| cg27312338 | 57832198 | + | N_Shelf | KIFC3(body); KIFC3(tss1500) |
| cg26993965 | 76617016 | + | Island | |
| cg00800679 | 15940058 | − | | FGFBP1(body) |
| cg08985785 | 39196340 | + | N_Shore | KCNK5(body) |
| cg15014458 | 43969810 | + | S_Shore | LYPD3(body) |
| cg08260052 | 3304449 | + | Island | MEFV(body) |
| cg03020006 | 2.02E+08 | − | Island | ALS2CR11(body) |
| cg19730268 | 71022823 | − | | |
| cg04232360 | 2792744 | + | | |
| cg00648184 | 60828157 | − | | MARCH10(body); MIR548W(body) |
| cg06769994 | 48288538 | − | | |
| cg02847897 | 88142275 | − | S_Shore | KLHL8(tss1500) |
| cg08777316 | 1.59E+08 | − | Island | |
| cg05886689 | 51051890 | − | Island | LRRC4B(body) |
| cg21199045 | 76324738 | − | S_Shore | |
| cg16651537 | 51226536 | − | N_Shore | CLEC11A(tss200) |
| cg23257225 | 2.03E+08 | + | | ADORA1(body) |
| cg08820801 | 39465821 | + | N_Shore | FBXO17(body) |
| cg04872675 | 1.09E+08 | + | | |
| cg25322720 | 19628292 | + | Island | |
| cg26122451 | 92672950 | − | Island | |
| cg26875637 | 1.13E+08 | + | N_Shore | CTTNBP2NL(tss1500) |
| cg12893736 | 72429636 | + | S_Shore | GPRC5C(body); GPRC5C(tss200) |
| cg08293242 | 1.13E+08 | − | Island | |
| cg10589385 | 1.51E+08 | + | N_Shore | SETDB1(tss1500) |
| cg09948179 | 42328988 | − | | PLA2G4E(body) |
| cg10208897 | 1.79E+08 | − | Island | ADAMTS2(body) |
| cg02311932 | 17282853 | + | S_Shore | RBM24(body); RBM24(tss200) |
| cg17930194 | 1.25E+08 | + | Island | LHX6(body) |
| cg26840462 | 1.25E+08 | − | | PKNOX2(body) |
| cg18264486 | 31650916 | − | Island | |
| cg01443452 | 36024439 | − | Island | GAPDH5(body) |
| cg16691177 | 1.66E+08 | + | | TRIM61(body) |
| cg23916496 | 85997314 | − | Island | LRIT1(body) |
| cg01189915 | 1.28E+08 | − | N_Shore | HILPDA(tss1500) |
| cg17382918 | 57042544 | − | Island | APCDD1L(body) |
| cg23093692 | 2.34E+08 | − | Island | SLC35F3(body) |
| cg20996561 | 40082316 | + | Island | CACNA1I(body) |
| cg07498426 | 1.26E+08 | + | | |
| cg04313735 | 73357194 | + | | PLEKHB1(tss1500); PLEKHB1(tss200) |
| cg18033416 | 30592033 | + | N_Shore | RHBDL3(tss1500) |
| cg26773791 | 94883242 | − | N_Shore | |
| cg18385190 | 1.05E+08 | + | Island | |
| cg06211243 | 12907446 | − | N_Shelf | |
| cg06378100 | 48656724 | − | | |
| cg21653641 | 49222892 | + | N_Shore | MAMSTR(body) |
| cg14289461 | 1.13E+08 | − | S_Shore | LAMA4(tss200) |
| cg01266390 | 1.25E+08 | + | | |
| cg04131405 | 48051691 | + | S_Shore | DLX4(body) |
| cg12416033 | 36484731 | + | N_Shore | SDHAF1(tss1500) |
| cg16851282 | 3254011 | + | Island | MRGPRE(tss1500) |
| cg06784539 | 1.38E+08 | + | | ATP6V0A4(tss200); TMEM213(body) |
| cg11909574 | 58598338 | + | | |
| cg09884310 | 27065428 | − | S_Shore | |
| cg16658020 | 39203641 | + | | KRTAP2-1(tss200) |
| cg04259752 | 23262159 | − | S_Shore | LOXL2(tss1500) |
| cg01268571 | 1.71E+08 | + | Island | |
| cg25249286 | 2.43E+08 | − | S_Shelf | |
| cg21313071 | 49640589 | + | S_Shore | KCNG1(tss1500) |
| cg21248196 | 23714084 | − | S_Shore | PSMA8(body) |
| cg26688911 | 39969536 | + | S_Shore | LPIN3(tss200) |
| cg25217583 | 50574383 | + | | ELAVL4(body); ELAVL4(tss1500) |
| cg08802482 | 2657119 | − | | |
| cg13673514 | 1.01E+08 | + | S_Shore | BEGAIN(body); BEGAIN(tss1500) |
| cg20764575 | 24797884 | + | S_Shore | DFNA5(tss1500) |
| cg08836375 | 1.64E+08 | + | Island | |
| cg08420135 | 52717725 | + | S_Shelf | |
| cg10528576 | 1.01E+08 | − | Island | DLK1(tss1500) |
| cg18242139 | 50574837 | + | | ELAVL4(body) |
| cg19380499 | 1.29E+08 | − | Island | EFCC1(body) |
| cg00009053 | 61283865 | − | Island | SYT7(body) |
| cg24015889 | 2951719 | + | N_Shore | |
| cg00700487 | 1.51E+08 | + | Island | NOS3(body) |
| cg08121652 | 48381824 | − | | TRABD2B(body) |
| cg22398616 | 53314203 | − | S_Shore | LECT1(tss1500) |
| cg12468238 | 3598319 | + | Island | IRX1(body) |
| cg13622265 | 1.25E+08 | − | | TTLL11(body) |
| cg16987606 | 72426469 | + | N_Shore | GPRC5C(tss1500) |
| cg26312935 | 59279406 | + | N_Shore | RNF111(tss1500) |
| cg16715722 | 1.03E+08 | + | N_Shore | TLX1(tss1500); TLX1NB(body) |
| cg12793096 | 1.64E+08 | − | Island | |
| cg13475995 | 5882576 | − | Island | |
| cg16813662 | 1.01E+08 | + | Island | LINC00554(tss1500) |
| cg03907016 | 6534760 | − | Island | TNFSF9(body) |
| cg10461261 | 78109450 | + | N_Shore | |
| cg07195674 | 1.06E+08 | − | Island | |
| cg17181653 | 1.44E+08 | + | S_Shore | SF3B5(tss1500) |
| cg02475301 | 3253838 | − | Island | MRGPRE(tss1500) |
| cg12672189 | 32427868 | − | | |
| cg18049167 | 32121261 | − | N_shore | LOC100507547(body); PPT2(body); |
| | | | | PPT2(tss1500); PPT2- |
| | | | | EGFL8(tss1500) |
| cg03890167 | 2.03E+08 | + | Island | |
| cg16454130 | 73245439 | − | Island | CLDN4(body) |
| cg04683509 | 45885800 | + | Island | PPP1R13L(body) |
| cg22694931 | 1.69E+08 | + | | DACT2(body) |
| cg20149170 | 23105148 | − | | EPHB2(body) |
| cg01690119 | 88511822 | − | N_Shore | |
| cg04964333 | 39969604 | − | S_Shore | LPIN3(body) |
| cg23058194 | 65369127 | + | Island | KBTBD13(tss200) |
| cg00771642 | 48988436 | − | Island | |
| cg19497998 | 26595126 | − | N_Shore | |
| cg11364888 | 1.17E+08 | − | | |
| cg16440561 | 2.2E+08 | + | Island | SPEG(body) |
| cg10137597 | 22132714 | + | N_Shore | PIWIL2(tss1500); PIWIL2(tss200) |
| cg26530345 | 2.2E+08 | − | N_Shore | SPEG(body) |
| cg07740110 | 3439203 | − | S_Shore | TRPV3(body) |
| cg09103073 | 39019724 | − | Island | KRT12(body) |
| cg16405908 | 3253896 | − | Island | MRGPRE(tss1500) |
| cg08661360 | 32016247 | + | | TNXB(body) |
| cg03555881 | 31275551 | − | N_Shore | |
| cg24728050 | 3253797 | − | Island | MRGPRE(tss200) |
| cg11043571 | 1.66E+08 | + | S_Shore | CPE(body) |
| cg15899727 | 1003529 | − | Island | |
| cg07688931 | 1.22E+08 | + | N_Shelf | |
| cg19659004 | 1.03E+08 | − | N_Shore | AMN(tss200) |
| cg14223654 | 45397802 | + | Island | |
| cg06720467 | 27189440 | − | N_Shore | SFN(tss200) |
| cg26304689 | 1.73E+08 | − | | |
| cg09159285 | 6664134 | + | S_Shore | KLHL21(tss1500) |
| cg19702802 | 46417729 | − | Island | NANOS2(body) |
| cg09842196 | 47012543 | − | S_Shore | |
| cg05079544 | 48644408 | − | S_Shore | N4BP1(tss1500) |
| cg16653901 | 1.34E+08 | − | S_Shore | SKP1(tss1500) |
| cg05530630 | 55224743 | − | | EGFR(body) |
| cg17820828 | 2813298 | + | Island | KCNQ1(body) |
| cg07008193 | 46318527 | − | S_Shore | CREB3L1(body) |
| cg01917340 | 90720130 | + | N_Shelf | |
| cg00455331 | 2.42E+08 | − | N_Shore | |
| cg05543030 | 45005364 | − | | |
| cg09826056 | 78511360 | + | N_Shore | GIPC2(tss1500) |
| cg06691250 | 7517807 | + | | |
| cg05329578 | 2241688 | + | N_Shore | |
| cg16107528 | 8928451 | + | S_Shore | NTN1(body) |
| cg01095004 | 88454883 | − | S_Shore | |
| cg05096467 | 56744490 | + | | TEX14(body) |
| cg00169782 | 39791734 | + | | KRT42P(body) |
| cg01965673 | 1.27E+08 | + | N_Shore | |
| cg23591611 | 1.33E+08 | − | Island | |
| cg17590162 | 63827361 | − | | MACROD1(body) |
| cg19307528 | 74004928 | − | N_Shore | SBSPON(body) |
| cg25530735 | 6572733 | + | | |
| cg24880736 | 1.57E+08 | − | N_Shore | |
| cg22033281 | 1.11E+08 | − | | |
| cg09201238 | 79486912 | + | Island | |
| cg21291672 | 29451713 | + | Island | TMEM200B(tss1500) |
| cg16071029 | 1594034 | − | | |
| cg08175536 | 89927750 | + | | LINC00925(body) |
| cg01114163 | 1856713 | − | N_Shore | |
| cg09511741 | 49668729 | − | N_Shore | TRPM4(body) |
| cg16154524 | 26633895 | − | Island | KRT18P55(body) |
| cg13734535 | 1.33E+08 | − | | NCS1(body) |
| cg26572207 | 20161947 | + | S_Shore | |
| cg10132767 | 1.68E+08 | − | | |
| cg07814122 | 13196942 | + | N_Shore | KIAA1467(tss1500) |
| cg10799440 | 1.21E+08 | + | | |
| cg04781339 | 44781686 | − | N_Shore | |
| cg06949812 | 38517993 | + | N_Shore | GJD3(body) |
| cg13695646 | 78407509 | − | | BHMT(tss200) |
| cg12458966 | 2.24E+08 | + | | |
| cg05804139 | 1321767 | − | S_Shelf | |
| cg04986504 | 12031266 | + | S_Shore | DKK3(tss1500) |
| cg03492827 | 72838601 | + | Island | |
| cg07923390 | 35109242 | + | Island | TCP11(tss200) |
| cg22138002 | 44237002 | − | Island | LOXHD1(tss200) |
| cg21615654 | 4213084 | + | | |
| cg11985839 | 30853007 | − | S_Shore | DDR1(body) |
| cg25923214 | 74864205 | − | Island | MGAT5B(tss1500) |
| cg08031492 | 469214 | − | S_Shore | |
| cg10265365 | 42827690 | + | N_Shore | TMEM145(body) |
| cg24371114 | 24913973 | + | | CCDC149(body) |
| cg10455971 | 1.01E+08 | − | | |
| cg05551068 | 1.18E+08 | − | N_Shore | RFC5(tss1500) |
| cg14942906 | 67384040 | − | S_Shore | DOC2GP(tss1500) |
| cg15015426 | 1.6E+08 | + | | OR10J5(tss1500) |
| cg01568317 | 74895911 | − | S_Shelf | |
| cg12987312 | 1.43E+08 | + | N_Shore | |
| cg04321555 | 34285737 | − | N_Shore | |
| cg03987884 | 61616862 | − | | HMSD(body) |
| cg01444808 | 1.32E+08 | − | S_Shore | SEPT8(tss1500) |
| cg20078406 | 46875709 | + | Island | PRSS42(tss200) |
| cg04120272 | 37123843 | + | | FBXO47(tss200) |
| cg16425726 | 83680145 | + | | SCD5(body) |
| cg04471507 | 26233376 | + | Island | MIR3917(tss1500); STMN1(tss1500); |
| | | | | STMN1(tss200) |
| cg26636010 | 20306539 | + | | |
| cg27194211 | 4503171 | − | S_Shelf | SMTNL2(body) |
| cg03638715 | 4665704 | + | Island | UBALD1(tss1500) |
| cg01806151 | 2470965 | − | N_Shore | |
| cg15344726 | 20717346 | + | Island | GJA3(body) |
| cg10279314 | 27185896 | − | | |
| cg23789054 | 28832537 | + | S_Shore | LOC401242(tss1500) |
| cg09033333 | 1.34E+08 | − | | |
| cg08414643 | 1.54E+08 | − | Island | |
| cg22952917 | 69325449 | + | Island | MIR548H4(body); NOX5(body) |
| cg03800939 | 1.25E+08 | + | S_Shore | SLC37A2(body) |
| cg22478544 | 980841 | − | | |
| cg11747081 | 76444823 | − | | C13orf45(tss1500) |
| cg21488617 | 38022586 | − | Island | DNALI1(body) |
| cg18717329 | 76779341 | + | | |
| cg06470855 | 1.13E+08 | − | Island | |
| cg09556447 | 36927100 | − | Island | PI16(body) |
| cg14701108 | 3111293 | − | | |
| cg05708536 | 1.13E+08 | + | N_Shore | |
| cg09576762 | 99548859 | + | | PGPEP1L(body) |
| cg14637144 | 1.97E+08 | + | | MFI2(body) |
| cg01759672 | 1.38E+08 | − | Island | GFRA3(body) |
| cg04017036 | 73104388 | − | S_Shelf | |
| cg02888048 | 98335163 | − | | |
| cg25470758 | 31280143 | − | Island | |
| cg10313047 | 52351355 | − | | DNAH1(body) |
| cg25011257 | 2193187 | + | | MIR4686(tss1500); TH(tss200) |
| cg10409299 | 1.5E+08 | + | S_Shore | TMEM176A(body); TMEM176B(tss1500) |
| cg03444077 | 45359721 | − | N_Shore | TMEM72-AS1(body) |
| cg21761639 | 31533786 | + | | |
| cg14476358 | 1.27E+08 | − | N_Shelf | |
| cg13422817 | 4550927 | + | N_Shelf | FGF6(body) |
| cg03238581 | 76006143 | + | | CSPG4(tss1500) |
| cg05928980 | 29567425 | − | S_Shelf | PTPRU(body) |
| cg05728902 | 57849029 | − | | INHBE(tss200) |
| cg21607649 | 1.35E+08 | + | N_Shore | |
| cg26077005 | 27236793 | + | | |
| cg05517791 | 73157351 | + | S_Shelf | |
| cg14020904 | 77019779 | − | N_Shore | C1QTNF1(body); C1QTNF1(tss1500); |
| | | | | C1QTNF1-AS1(body) |
| cg11625488 | 1.7E+08 | + | S_Shore | |
| cg07803420 | 30851517 | + | N_Shore | DDR1(tss1500) |
| cg08370430 | 12927578 | + | | |
| cg05338187 | 2.37E+08 | + | N_Shore | LGALS8(body); LGALS8(tss1500); LGALS8 |
| | | | | (tss200); LGALS8-AS1(body) |
| cg00555085 | 87616248 | + | | |
| cg09468051 | 41879262 | + | N_Shore | |
| cg05653018 | 2.02E+08 | + | | ELF3(tss200) |
| cg27008363 | 54449761 | + | S_Shore | HOXC4(body) |
| cg05628160 | 54785374 | − | | RGS20(body) |
| cg08000731 | 316247 | − | Island | |
| cg24254488 | 25991654 | − | S_Shore | |
| cg26817877 | 1.74E+08 | + | | |
| cg14673415 | 1.13E+08 | + | N_Shore | |
| cg04512603 | 64345267 | − | | |
| cg05312960 | 25159879 | + | Island | PIWIL3(body); TOP1P2(tss1500) |
| cg11229864 | 58668068 | − | | |
| cg07555084 | 27186199 | − | | |
| cg15778434 | 54493114 | + | N_Shore | CACNG6(tss1500); CACNG8(body) |
| cg16680214 | 1.55E+08 | − | | KCNN3(body) |
| cg06093039 | 1.32E+08 | − | N_Shelf | GPR133(body) |
| cg18223453 | 59554746 | + | | TBX4(body) |
| cg14858616 | 72219819 | − | S_Shore | |
| cg03252627 | 57029877 | + | N_Shore | |
| cg11196848 | 16859833 | − | S_Shore | FGF20(tss200) |
| cg14488962 | 1.32E+08 | + | | NTM(body) |
| cg02826525 | 39186885 | − | Island | ARHGEF33(body); LOC375196(body) |
| cg03451178 | 1.73E+08 | + | N_Shore | METAP1D(tss1500) |
| cg20488756 | 30131586 | + | | TRIM15(body) |
| cg09879253 | 45935359 | + | | |
| cg23628563 | 1.12E+08 | + | | |
| cg24867279 | 28853021 | − | | |
| cg03890691 | 30023615 | + | S_Shore | DOC2A(body); DOC2A(tss1500) |
| cg20816104 | 1.32E+08 | − | | |
| cg21205031 | 32502699 | − | N_Shore | YIPF4(tss1500) |
| cg01258050 | 1.35E+08 | − | | KNDC1(body) |
| cg09259772 | 12726089 | + | N_Shore | ARL4A(tss1500) |
| cg20675702 | 13200491 | + | N_Shelf | |
| cg06835125 | 1.06E+08 | − | | |
| cg00901554 | 1042225 | − | N_Shore | |
| cg21593676 | 88142277 | − | S_Shore | KLHL8(tss1500) |
| cg19706795 | 24797839 | − | S_Shore | DFNA5(tss1500); DFNA5(tss200) |
| cg18031326 | 68079681 | + | N_Shore | LRP5(tss1500) |
| cg08818829 | 13810279 | + | Island | DNAH5(body) |
| cg03755115 | 76858729 | − | Island | MYO7A(body) |
| cg14640244 | 1.4E+08 | − | S_Shelf | LCN10(body) |
| cg01475377 | 1.1E+08 | − | Island | |
| cg10115348 | 17865653 | − | N_Shore | ARHGEF10L(tss1500) |
| cg18103394 | 47433374 | − | N_Shore | SYN1(body) |
| cg04104776 | 3439336 | + | S_Shore | TRPV3(body) |
| cg16529323 | 20137449 | + | N_Shelf | |
| cg02151983 | 1.95E+08 | + | | |
| cg00301396 | 30572929 | − | Island | |
| cg21497977 | 1.59E+08 | + | S_Shore | |
| cg04617936 | 214353 | − | S_Shore | |
| cg11482879 | 4480083 | − | | |
| cg16189953 | 76674571 | + | Island | |
| cg26923084 | 52536893 | + | S_Shore | NID2(tss1500) |
| cg05211480 | 21148814 | − | N_Shelf | |
| cg14210678 | 1.29E+08 | + | N_Shelf | LMX1B(body) |
| cg07311419 | 79197548 | − | | |
| cg26877596 | 1.07E+08 | + | S_Shore | NUAK1(tss1500) |
| cg14191654 | 44829602 | + | Island | CDH22(body) |
| cg23129573 | 73442487 | − | | ELN(body) |
| cg23424043 | 1.39E+08 | + | S_Shore | ZC3HAV1(tss1500) |
| cg05184311 | 2.01E+08 | − | Island | PKP1(body) |
| cg19524572 | 95156762 | + | | |
| cg05100432 | 1.05E+08 | − | | |
| cg02677192 | 45138294 | − | Island | PDXK(tss1500) |
| cg16672421 | 2.29E+08 | − | S_Shore | CCSAP(tss1500) |
| cg20945085 | 74875227 | − | Island | M1AP(body); M1AP(tss200) |
| cg18120323 | 1978732 | + | N_Shelf | |
| cg02602264 | 1026310 | + | Island | CYP2W1(body) |
| cg18680834 | 30863349 | + | N_Shelf | ZNF536(body) |
| cg18061197 | 1.01E+08 | − | Island | RGS22(body); RGS22(tss200) |
| cg25037335 | 740043 | − | S_Shelf | ERICH1-AS1(body) |
| cg16328007 | 41119988 | + | Island | RIMS3(body) |
| cg16532282 | 2.26E+08 | + | Island | |
| cg22122321 | 85407081 | − | S_Shore | |
| cg02828104 | 48770800 | − | S_Shore | TMEM189(tss1500); TMEM189- |
| | | | | UBE2V1(tss1500) |
| cg04637454 | 41651066 | − | Island | |
| cg15542459 | 1.3E+08 | + | Island | |
| cg19180828 | 6265220 | + | N_Shore | RNF207(tss1500) |
| cg05726758 | 88448456 | − | N_Shore | |
| cg03270204 | 30851638 | − | N_Shore | DDR1(tss1500) |
| cg14992524 | 10586135 | − | N_Shore | SOX7(body) |
| cg07138793 | 28148330 | − | Island | OCA2(body) |
| cg13809095 | 65647092 | − | | |
| cg01963623 | 18632433 | − | N_Shore | USP18(tss1500) |
| cg24479014 | 1.03E+08 | + | | WTAPP1(body) |
| cg02854229 | 1.02E+08 | − | | |
| cg10173814 | 1.28E+08 | − | Island | |
| cg17579753 | 34079998 | + | | GAS2L2(tss200) |
| cg22894896 | 29886890 | − | Island | MIR193A(tss200) |
| cg00660106 | 1.19E+08 | − | Island | KIAA1598(body) |
| cg17867243 | 42371653 | − | Island | PLA2G4D(body) |
| cg20862097 | 39760776 | − | S_Shore | DAAM2(body) |
| cg24332577 | 50419248 | − | S_Shore | SALL4(tss200) |
| cg08602346 | 1.13E+08 | + | S_Shore | TUBGCP3(tss1500) |
| cg04800569 | 55888003 | − | N_Shore | TMEM190(tss1500) |
| cg00930707 | 2.02E+08 | + | N_Shore | RNPEP(tss1500) |
| cg18665594 | 1.01E+08 | + | | |
| cg27282281 | 1.12E+08 | − | | |
| cg00907200 | 2.39E+08 | − | Island | ESPNL(body) |
| cg08422645 | 2.1E+08 | − | | |
| cg03332701 | 63793652 | − | N_Shelf | |
| cg18310895 | 62086195 | + | S_Shore | KCNQ2(body) |
| cg21964928 | 1.54E+08 | − | Island | |
| cg11797717 | 43198278 | − | Island | PLCD3(body) |
| cg20893919 | 1.23E+08 | + | Island | TRPC3(body) |
| cg11382666 | 20032841 | + | | |
| cg14354168 | 1619134 | − | Island | |
| cg19802390 | 2840177 | − | | |
| cg09223687 | 1027885 | + | Island | |
| cg09784886 | 71293996 | − | | KRTAP5-11(tss200) |
| cg02483204 | 1.17E+08 | − | | |
| cg19701087 | 52859008 | + | N_Shore | GSTA4(body) |
| cg20516209 | 27301943 | + | N_Shelf | EMILIN1(body) |
| cg15283852 | 9550296 | − | S_Shore | USP43(body) |
| cg03703111 | 92925721 | − | S_Shore | MIR548AO(body); NR2F1(body) |
| cg17156402 | 16390468 | + | | FAM131C(body) |
| cg10888244 | 1.61E+08 | + | Island | PVRL4(body) |
| cg01991625 | 54404353 | − | | |
| cg22157494 | 3774827 | + | N_Shore | CEP104(tss1500); DFFB(body) |
| cg20752878 | 7080538 | + | S_Shelf | ASGR1(body) |
| cg18964375 | 33772147 | + | Island | |
| cg06699564 | 95651099 | − | N_Shore | LOC100288748(body) |
| cg19769301 | 1.49E+08 | − | N_Shore | NBPF23(body) |
| cg27387193 | 32064032 | + | Island | TNXB(body) |
| cg24368876 | 1.71E+08 | − | | |
| cg12689679 | 31516336 | − | | LOC283710(body) |
| cg19490561 | 10971308 | − | | PDIA6(body) |
| cg02780269 | 80292252 | − | Island | SECTM1(tss1500) |
| cg25782983 | 74950764 | + | N_Shore | TPBGL(tss1500) |
| cg17201651 | 1.12E+08 | + | S_Shore | XPNPEP1(tss1500) |
| cg26600802 | 1.16E+08 | − | Island | TDRD1(body) |
| cg00839584 | 1.14E+08 | − | | IL1A(body) |
| cg19163067 | 36042438 | − | Island | TFAP2E(body) |
| cg10643271 | 1.29E+08 | + | S_Shore | SLC15A4(tss1500) |
| cg23927469 | 6909853 | − | N_Shore | TBC1D14(tss1500) |
| cg13749379 | 41833161 | − | Island | SOST(body) |
| cg21844144 | 68920550 | + | | LOC338694(body) |
| cg12097325 | 1076431 | − | S_Shelf | LOC254099(body) |
| cg21811204 | 1.32E+08 | − | Island | SHROOM1(body) |
| cg05866737 | 1650863 | + | | KRTAP5-5(tss200); MOB2(body) |
| cg14463912 | 1.03E+06 | + | Island | PDZD7(body); SFXN3(tss1500) |
| cg18197594 | 94334836 | + | S_Shore | IDE(tss1500) |
| cg22047901 | 1.29E+08 | + | N_Shelf | MIR183(tss1500); MIR96(tss1500) |
| cg04839131 | 1.51E+08 | + | Island | XCNH2(body) |
| cg19843891 | 48675647 | + | N_Shore | |
| cg13630131 | 25891154 | − | N_Shore | |
| cg02174092 | 43846539 | + | Island | |
| cg02664787 | 1.33E+08 | − | S_Shore | OPCML(body) |
| cg04872541 | 1.02E+08 | − | | PKD2L1(tss200) |
| cg06814287 | 32120584 | + | N_Shore | LOC100507547(body); PPT2(tss1500); |
| | | | | PPT2- |
| | | | | EGFL8(tss1500); PRRT1(rss1500) |
| cg13504055 | 1.02E+08 | + | | |
| cg22369590 | 1.57E+08 | + | N_Shore | HAPLN2(body) |
| cg13904562 | 48690974 | + | | |
| cg08021797 | 85785130 | + | Island | C16orf74(tss1500) |
| cg27612019 | 18064052 | − | Island | SLC25A18(body) |
| cg12070285 | 74072447 | − | N_Shore | KHDC3L(body) |
| cg01783554 | 1.02E+08 | − | | |
| cg23144063 | 58570828 | − | N_Shore | FAM107A(body) |
| cg10336790 | 1.13E+08 | − | | GPR85(body) |
| cg11789681 | 74046708 | + | S_Shore | |
| cg20929609 | 40626736 | − | N_Shore | RLF(tss1500) |
| cg11194698 | 44184840 | + | Island | GCK(body) |
| cg17168157 | 1.01E+08 | + | S_Shore | RGS22(tss1500) |
| cg24919768 | 1.11E+08 | + | Island | MALL(body) |
| cg07755203 | 1.5E+08 | − | Island | |
| cg11950360 | 34834690 | + | S_Shore | CELF4(body) |
| cg03184588 | 31480300 | − | S_Shore | FAM60A(tss1500) |
| cg02198562 | 1.35E+08 | + | N_Shore | |
| cg13092405 | 2.28E+08 | − | Island | |
| cg03015498 | 7517765 | + | | |
| cg08942800 | 49681310 | + | | CRISP2(tss200) |
| cg03724964 | 39465969 | + | Island | FBXO17(body) |
| cg14716095 | 20968690 | − | N_Shore | |
| cg12552293 | 36997731 | − | S_Shore | C17orf98(tss200) |
| cg25696972 | 1594827 | + | | |
| cg03211420 | 50821399 | − | S_Shelf | |
| cg11433098 | 99987894 | + | | |
| cg04254769 | 1.42E+08 | + | | |
| cg14266410 | 92036630 | − | N_Shelf | |
| cg26286839 | 79378955 | − | | THBS4(body) |
| cg16919708 | 1.33E+08 | + | | OPCML(body) |
| cg20636352 | 2E+08 | − | Island | |
| cg13906860 | 85393895 | − | | |
| cg04249191 | 27384423 | − | | |
| cg08635489 | 10949944 | + | | |
| cg12828018 | 59437416 | − | S_Shore | PATL1(tss1500) |
| cg20673819 | 1.66E+08 | + | Island | LINC00473(body) |
| cg16610412 | 1.33E+08 | + | | FSTL4(body) |
| cg14825555 | 27190639 | − | N_Shore | SFN(body) |
| cg20467658 | 89933347 | − | | LINC00925(body) |
| cg05930207 | 38518520 | − | N_Shore | GJD3(body) |
| cg07880854 | 1.13E+08 | − | N_Shore | FBLN7(tss1500) |
| cg15849368 | 1811181 | − | N_Shore | |
| cg16761096 | 51383119 | − | N_Shore | COBL(body) |
| cg24716558 | 72915784 | − | Island | USH1G(body) |
| cg24179445 | 1.02E+08 | + | N_Shore | |
| cg18105179 | 2.43E+08 | + | N_Shore | GAL3ST2(body) |
| cg20714328 | 79331850 | + | S_Shore | THBS4(body) |
| cg13269964 | 41924577 | + | | CD300LG(body) |
| cg22233974 | 39546511 | − | N_Shore | |
| cg19821361 | 46012520 | − | | KRTAP10-6(tss200); TSPEAR(body) |
| cg23009419 | 46618597 | − | Island | TDGF1(body); TDGF1(tss1500) |
| cg27551856 | 87250689 | − | Island | |
| cg10093648 | 29912700 | − | N_Shore | ASPHD1(body) |
| cg12875241 | 9734063 | − | Island | GPR143(tss200) |
| cg00720629 | 19752228 | + | N_Shore | TBX1(body) |
| cg05756954 | 8751266 | − | N_Shore | |
| cg07173688 | 1.2E+08 | − | | |
| cg16343401 | 42989147 | − | Island | GFAP(body) |
| cg01675158 | 99988776 | + | | CCDC85C(body) |
| cg11654325 | 2.43E+08 | − | S_Shelf | CXXC11(tss1500) |
| cg14814062 | 54927903 | − | Island | TTYH1(body) |
| cg22728830 | 58667964 | + | | |
| cg04284140 | 44163975 | + | S_Shore | POLD2(tss1500) |
| cg26250086 | 6830972 | − | | |
| cg12613020 | 1.35E+08 | − | S_Shore | |
| cg03540622 | 42706066 | + | | ZBTB47(body) |
| cg10621809 | 95840313 | − | | |
| cg23681730 | 35683551 | + | N_Shore | TRIM44(tss1500) |
| cg16171484 | 2.23E+08 | + | S_Shore | SGPP2(body) |
| cg23648082 | 2517142 | + | | TMC2(tss200) |
| cg01346718 | 38713874 | − | S_Shore | CSNK1E(body); CSNK1E(tss1500) |
| cg08770961 | 66956122 | + | Island | RRAD(body) |
| cg11794215 | 35423816 | − | | |
| cg08370178 | 43034043 | − | N_Shelf | |
| cg16481280 | 32120955 | + | N_Shore | LOC100507547(body); PPT2(tss1500); |
| | | | | PPT2- |
| | | | | EGFL8(tss1500); PRRT1(tss1500) |
| cg18129759 | 2.02E+08 | − | | GPR37L1(tss200) |
| cg18537730 | 49250080 | − | N_Shelf | IZUMO1(body) |
| cg07504598 | 24797786 | − | S_Shore | DFNA5(tss1500); DFNA5(tss200) |
| cg16537292 | 686186 | − | N_Shore | ERICH1-AS1(tss1500) |
| cg14585415 | 1.03E+08 | − | Island | PDZD7(body) |
| cg05130518 | 21295590 | − | N_Shore | IL17D(body) |
| cg00057231 | 48631958 | − | Island | COL7A1(body) |
| cg05293216 | 35080812 | − | S_Shore | FANCG(tss1500) |
| cg02012483 | 30131570 | − | | TRIM15(body) |
| cg03499943 | 1.26E+08 | − | | LHPP(body) |
| cg22738655 | 13276306 | − | | UCMA(body) |
| cg12899423 | 44328154 | − | S_Shore | ALX4(body) |
| cg04307107 | 50180603 | + | S_Shore | NFATC2(tss1500) |
| cg18410945 | 1.57E+08 | + | N_Shelf | |
| cg07262335 | 85465018 | − | | TCF7L1(body) |
| cg17463698 | 83477712 | − | N_Shore | WHAMM(tss1500) |
| cg15731001 | 1.53E−08 | + | | SLC6A8(body); SLC6A8(tss200) |
| cg17862447 | 23633680 | + | | KLHL29(body) |
| cg13689996 | 71810497 | − | | LINC00469(body) |
| cg12197752 | 1.01E−08 | − | | |
| cg00642615 | 69590937 | + | N_Shore | PAQR5(tss1500) |
| cg08122232 | 74100132 | − | S_Shore | EXOC7(tss1500) |
| cg14089267 | 23412409 | − | Island | |
| cg02304584 | 31650790 | − | Island | |
| cg13269555 | 1.03E+08 | − | Island | KAZALD1(body) |
| cg02275035 | 70327025 | − | | |
| cg23075506 | 2.13E+08 | + | S_Shore | |
| cg03871267 | 67483475 | − | | |
| cg25201101 | 1.31E+08 | − | N_Shore | RAN(tss1500) |
| cg01881287 | 1.05E+08 | + | Island | |
| cg27416261 | 35406590 | − | | ATP8B5P(tss200) |
| cg24415728 | 11471816 | − | S_Shore | |
| cg10831285 | 17918927 | + | Island | B3GNT3(body) |
| cg12078872 | 1.11E+08 | + | Island | DDO(body) |
| cg26750002 | 49244674 | − | S_Shore | IZUMO1(body); RASIP1(tss1500) |
| cg08947894 | 1.55E+08 | + | S_Shore | |
| cg21566642 | 2.33E+08 | − | Island | |
| cg16166968 | 1.2E+08 | − | Island | ADAM30(body) |
| cg00583861 | 32014363 | + | | |
| cg19733042 | 1.48E+08 | + | | NBPF8(body) |
| cg20743080 | 1.86E+08 | − | N_Shore | |
| cg12020444 | 1.31E+08 | − | N_Shore | PIWIL1(tss1500) |
| cg12209693 | 73894884 | + | Island | GTF2IRD1(body) |
| cg25212270 | 78015279 | + | | |
| cg12024822 | 90597394 | + | Island | |
| cg06406026 | 1.32E+08 | − | S_Shore | |
| cg22105158 | 3480672 | + | N_Shore | C19orf77(tss200) |
| cg05090351 | 1.27E+08 | − | Island | |
| cg23553912 | 1.6E+08 | − | | IQCJ-SCHIP1(body); SCHIP1(body) |
| cg16652937 | 23243625 | − | N_Shore | UBE2E2(tss1500) |
| cg19849544 | 38508335 | + | Island | |
| cg10430077 | 1.81E+08 | + | N_Shore | |
| cg15365218 | 33396859 | + | N_Shore | |
| cg24406754 | 64783110 | − | N_Shore | C12orf56(body) |
| cg12160664 | 1.15 E+08 | − | | OLFML3(tss200) |
| cg15442434 | 1.7E+08 | − | | |
| cg21378403 | 1.03E+08 | − | N_Shore | PDZD7(body) |
| cg24369989 | 78933807 | − | Island | CHRNB4(tss1500) |
| cg08638044 | 27819924 | − | | |
| cg25013303 | 10961257 | − | | |
| cg17099016 | 17568889 | + | S_Shelf | OTOG(tss200) |
| cg27031448 | 27998168 | + | N_Shore | GTF3A(tss1500) |
| cg22591480 | 91296252 | + | S_Shore | SLC16A12(tss1500) |
| cg18212064 | 1.43E+08 | + | | |
| cg04975966 | 1.11E−08 | − | N_Shore | ALX3(body) |
| cg03303857 | 1.21E+08 | + | | GRIK4(body) |
| cg23484899 | 2.45E+08 | + | Island | |
| cg20996314 | 21989292 | + | S_Shore | HR(tss1500) |
| cg27343801 | 46444017 | − | Island | NOVA2(body) |
| cg17623793 | 4230872 | − | | |
| cg26349358 | 468989 | + | S_Shore | |
| cg10256477 | 49934029 | + | S_Shore | KCNH3(body) |
| cg08087655 | 1.22E+08 | − | | MIR100HG(body) |
| cg20005057 | 10364834 | + | N_Shore | GABARAPL1(tss1500) |
| cg26644395 | 5407020 | + | | UCN3(body) |
| cg17506621 | 1.6E+08 | − | | |
| cg01795776 | 23003462 | + | Island | |
| cg27413508 | 30225517 | − | N_Shore | COX4I2(tss200) |
| cg00060374 | 1355235 | − | Island | ANKRD65(body) |
| cg07839926 | 1.22E+08 | − | Island | |
| cg17570276 | 6099250 | − | S_Shore | EIF2AK1(tss1500) |
| cg05045817 | 1.85E+08 | + | Island | |
| cg07480640 | 64211328 | + | | PRICKLE2(tss200) |
| cg20432350 | 1.12E+08 | − | | KCND3(body) |
| cg03025830 | 21905599 | + | Island | FGF17(body) |
| cg17571266 | 54763211 | − | Island | ZNF385A(body) |
| cg21729798 | 24357078 | + | N_Shore | DCDC2(body); KAAG1(tss200) |
| cg04349021 | 52281901 | − | | ANKRD33(body) |
| cg25943845 | 48416839 | + | S_Shelf | GDF2(body) |
| cg06159340 | 30907560 | + | S_Shore | CTF1(tss1500) |
| cg25745746 | 20617452 | − | | VWA5B1(body) |
| cg17908602 | 1.17E+08 | + | | |
| cg07102320 | 46171075 | + | | |
| cg08634357 | 66956242 | − | Island | RRAD(body) |
| cg07925738 | 16847839 | − | Island | |
| cg23006519 | 1.21E+08 | − | | LINC01101(body) |
| cg11613902 | 98156120 | − | | TLL2(body) |
| cg16511795 | 3535009 | − | S_Shore | LRPAP1(tss1500) |
| cg22816857 | 11565391 | − | Island | ELAVL3(body) |
| cg16009311 | 1.19E+08 | + | | EXT1(body) |
| cg04779752 | 73245457 | + | Island | CLDN4(body) |
| cg16079541 | 30848846 | + | N_Shelf | |
| cg18019825 | 1329016 | − | Island | |
| cg25956097 | 44163499 | + | Island | ZNF32-AS3(body) |
| cg05134847 | 19742426 | − | N_Shore | |
| cg19432362 | 46426263 | + | | SIAH3(tss1500) |
| cg27355653 | 89940260 | − | N_Shelf | LINC00925(body) |
| cg12444736 | 1.13E+08 | − | Island | |
| cg01016191 | 1.31E+08 | − | | |
| cg25035485 | 21266500 | + | N_Shore | APOB(body) |
| cg15630458 | 1.43E+08 | − | S_Shore | |
| cg15986841 | 53386900 | + | Island | ECHDC2(body) |
| cg07861603 | 3085246 | + | Island | CCDC64B(body); LOC100128770(body) |
| cg09720117 | 5152546 | + | N_Shore | KCNA5(tss1500) |
| cg13460311 | 72837782 | − | S_Shore | |
| cg08011245 | 55975827 | − | S_Shelf | |
| cg05815196 | 31536540 | − | | PLA2G3(tss200) |
| cg05442477 | 34444584 | − | S_Shore | OL1G1(body) |
| cg23553444 | 1.34E+08 | + | Island | GLB1L2(body) |
| cg01055594 | 20190349 | − | S_Shore | WDR35(tss1500) |
| cg02314896 | 49059802 | + | N_Shore | SULTZB1(body) |
| cg10769767 | 37024153 | + | N_Shore | PAX5(body) |
| cg27403609 | 11101403 | + | Island | |
| cg04279801 | 19314319 | − | | RNF112(tss200) |
| cg03460756 | 45353128 | + | | ITGB3(body) |
| cg03689146 | 52341810 | + | Island | |
| cg14036981 | 68920648 | − | | LOC338694(body) |
| cg00076774 | 45016971 | − | | |
| cg18821144 | 68881398 | + | | |
| cg10467371 | 1.68E−05 | − | | |
| cg08994082 | 2525459 | + | Island | GNG7(body) |
| cg07777909 | 21909645 | + | S_Shelf | DMTN(tss1500) |
| cg21723861 | 39686628 | + | S_Shelf | |
| cg23268630 | 32264315 | − | | SPOCD1(body) |
| cg24506936 | 1.44E+08 | + | Island | |
| cg19331988 | 88771031 | − | | |
| cg27239280 | 30907679 | + | S_Shore | CTF1(tss1500) |
| cg20636351 | 55087400 | + | | |
| cg03440414 | 11101592 | + | S_Shore | |
| cg14266237 | 12995624 | − | N_Shore | KLF1(body) |
| cg11651209 | 1.33E+08 | + | N_Shore | |
| cg22277296 | 54180170 | + | Island | |
| cg09225287 | 80549171 | + | S_Shore | SEMA3C(tss1500) |
| cg15142430 | 44498341 | − | S_Shore | PIAS2(tss1500) |
| cg05520687 | 62516325 | − | Island | |
| cg06706876 | 57317679 | + | N_shore | PLLP(body) |
| cg08415998 | 50602664 | − | N_Shore | DRGX(body) |
| cg18690729 | 1.28E−08 | − | | |
| cg21697812 | 33721714 | − | Island | C11orf91(body) |
| cg03467555 | 54404408 | + | | |
| cg14989226 | 75897850 | − | S_Shore | SRRM3(body) |
| cg18104354 | 1.06E+08 | − | | |
| cg06862049 | 49001890 | + | Island | LMTK3(body) |
| cg27340001 | 43786010 | − | S_Shore | RNU6- |
| | | | | 28P(body); TP53BPl(body); TP53BP1 |
| | | | | (tss1500) |
| cg03027739 | 1.28E+08 | + | N_Shore | |
| cg18534077 | 1.05E+08 | + | N_Shore | AS3MT(tss1500); C10orf32- |
| | | | | ASMT(body) |
| cg17307791 | 26806179 | + | | |
| cg11312495 | 1.16E+08 | − | Island | |
| cg22356117 | 1.83E+08 | − | S_Shore | LAMP3(tss1500) |
| cg25418852 | 35068221 | − | N_Shore | SCGB1B2P(body) |
| cg14302936 | 36135812 | − | N_Shore | GLIPR2(tss1500) |
| cg17516330 | 74494781 | − | | STRA6(body) |
| cg24117274 | 21945805 | − | N_Shelf | RAP1GAP(body) |
| cg10969241 | 1.32E+08 | + | S_Shore | |
| cg16810343 | 75898970 | + | S_Shelf | SRRM3(body) |
| cg03846408 | 1769390 | + | Island | IFITM10(body); MOB2(body) |
| cg01654820 | 10675123 | − | S_Shore | EMP2(tss1500) |
| cg07209550 | 79070077 | − | | CMYA5(body) |
| cg09982773 | 1.29E+08 | + | Island | KCP(tss200) |
| cg18498241 | 69286352 | − | Island | |
| cg19824710 | 43995379 | − | N_Shore | PTPRF(tss1500) |
| cg15151749 | 17520223 | − | | USH1C(body) |
| cg07421596 | 31913770 | − | | CFB(body) |
| cg23886216 | 92673517 | + | | |
| cg04983977 | 2.01E+08 | + | N_Shore | GPR25(body) |
| cg08750432 | 48676092 | + | N_Shore | |
| cg02931642 | 1.84E+08 | − | N_Shore | |
| cg01135546 | 45949828 | − | Island | |
| cg12159314 | 92787905 | + | N_Shore | SLC24A4(tss1500) |
| cg19247654 | 76617506 | + | Island | |
| cg04798919 | 44039475 | + | Island | ZNF575(body) |
| cg07972116 | 2822822 | + | | |
| cg21531689 | 2.2E+08 | + | S_Shore | DNPEP(tss1500) |
| cg05090695 | 2907670 | + | Island | CDKN1C(tss1500) |
| cg10245967 | 46966189 | − | | |
| cg19241831 | 26782726 | + | N_Shelf | OTOF(tss1500) |
| cg17938867 | 1.09E+08 | + | N_Shore | GPSM2(tss1500) |
| cg06023661 | 32164801 | + | S_Shore | NOTCH4(body) |
| cg06035600 | 27687454 | + | Island | MAP3K6(body) |
| cg19443750 | 1.13E+08 | + | N_Shore | |
| cg13019568 | 95826830 | + | Island | |
| cg04709703 | 61449898 | − | S_Shore | DAGLA(body) |
| cg17019053 | 74875263 | + | Island | M1AP(body); M1AP(tss200) |
| cg13293535 | 11597251 | − | | GATA4(body) |
| cg14339848 | 1.26E+08 | + | N_Shore | KLF15(body) |
| cg21904329 | 66549307 | − | S_Shelf | |
| cg02240291 | 1.51E+08 | + | N_Shore | SMARCD3(body) |
| cg10056813 | 1108860 | + | N_Shore | TTLL10(tss1500) |
| cg14492992 | 53101548 | + | S_Shelf | |
| cg16432009 | 37955936 | − | N_Shore | CDC42EP1(tss1500) |
| cg10997906 | 1.79E+08 | + | | ADAMTS2(body) |
| cg02687418 | 85393765 | + | | |
| cg21023520 | 43817384 | − | Island | MPPED1(body) |
| cg04021544 | 2705189 | − | S_Shelf | |
| cg07138994 | 8361530 | − | | |
| cg09565002 | 15939889 | + | | FGFBP1(body) |
| cg20903926 | 55271677 | − | Island | C1orf177(tss200) |
| cg18699242 | 18632429 | − | N_Shore | USP18(tss1500) |
| cg16703135 | 55517132 | + | Island | |
| cg10765171 | 23270757 | − | Island | |
| cg23366166 | 1.25E+08 | − | S_Shore | FEZ1(tss1500) |
| cg12944343 | 93698416 | − | | |
| cg14544666 | 1.8E+08 | + | Island | RASGEF1C(body) |
| cg00262344 | 23713741 | − | Island | PSMA8(tss200) |
| cg13476313 | 1.27E+08 | + | | NR5A1(body) |
| cg00116315 | 35466268 | + | Island | TEAD3(tss1500); TULP1(body) |
| cg19479373 | 99734154 | + | | CRTAC1(body) |
| cg15523463 | 1.51E+08 | + | | |
| cg25736663 | 2.34E+08 | + | S_Shore | SLC35F3(body) |
| cg07928883 | 1.49E+08 | + | S_Shore | |
| cg06472166 | 47783603 | − | | |
| cg24123106 | 1.23E+08 | − | | FABP7(tss200) |
| cg00904823 | 1.12E+08 | − | | RBM20(body) |
| cg05129050 | 7178026 | + | | CAMTA1(body) |
| cg23739457 | 1.17E+08 | − | | DSCAML1(body) |
| cg05670995 | 1.27E+08 | + | Island | MMP21(body) |
| cg16273979 | 1.22E+08 | + | | |
| cg05113697 | 53544098 | + | | |
| cg23765086 | 44348976 | − | N_Shore | CAMK2B(body) |
| cg02082083 | 20396560 | + | | PLA2GS(tss200) |
| cg21629821 | 46713473 | + | | DKFZp434J0226(tss200) |
| cg14847483 | 34516640 | − | | EMC4(tss1500) |
| cg20077028 | 52208899 | − | S_Shore | |
| cg13778078 | 785160 | − | N_Shore | CPLX1(body) |
| cg07525299 | 2293171 | − | S_Shore | ASCL2(tss1500) |
| cg05306173 | 1.19E+08 | − | Island | KIAA1598(body) |
| cg21428990 | 1.56E+08 | − | | |
| cg03421300 | 27189787 | − | N_Shore | SFN(body) |
| cg22038738 | 42064673 | − | | PLAT(body) |
| cg23621967 | 2.36E+08 | − | | |
| cg02238549 | 1.32E+08 | + | S_Shore | |
| cg00023919 | 66839191 | + | Island | RHOD(body) |
| cg17149262 | 37186030 | + | | |
| cg07856975 | 36356162 | + | S_Shore | ETV7(tss1500) |
| cg18730926 | 1.2E+08 | − | | PCDP1(body) |
| cg04258219 | 1.55E+08 | + | N_Shore | |
| cg21079003 | 93615146 | − | N_Shore | RGMA(body) |
| cg06071746 | 57297518 | + | N_Shore | GDPD1(tss1500) |
| cg27598583 | 86337662 | + | Island | |
| cg13826247 | 54424203 | + | N_Shore | HOXC4(body); HOXC5(body); HOXC6 |
| | | | | (body) |
| cg09584358 | 14201680 | − | N_Shore | |
| cg03721641 | 50451245 | + | N_Shore | IL17REL(tss200) |
| cg03874542 | 8338503 | − | | |
| cg01321174 | 1041044 | − | N_Shelf | |
| cg24231558 | 16626326 | − | Island | |
| cg14023999 | 90543224 | + | N_Shore | |
| cg05766510 | 1.57E+08 | − | Island | PTPRN2(body) |
| cg24553170 | 49243472 | + | S_Shore | AGBL4(body); BEND5(tss1500) |
| cg05336956 | 1098001 | + | N_Shore | |
| cg26839356 | 99603018 | − | S_Shore | |
| cg27532318 | 32358331 | + | | |
| cg11972810 | 76484379 | + | Island | C15orf27(body) |
| Cg01099024 | 70347833 | − | S_Shore | SMOC1(body) |
| cg02758247 | 59222050 | + | S_Shore | CDH20(body) |
| cg14413262 | 8384046 | − | N_Shore | SLC45A1(tss1500) |
| cg03268942 | 74148310 | + | | |
| cg27336261 | 1975562 | − | | |
| cg07201996 | 60348277 | + | Island | CDH4(body) |
| cg26411822 | 28099013 | + | N_Shore | STX12(tss1500) |
| cg24125828 | 32117049 | − | Island | PRRT1(body) |
| cg11858450 | 15132184 | + | Island | CCDC105(body) |
| cg15676542 | 26421231 | + | | BTN2A3P(tss1500) |
| cg22830895 | 1.51E+08 | + | S_Shore | CRYGN(tss1500) |
| cg04359306 | 88254988 | − | | |
| cg02939356 | 46875760 | − | Island | PRSS42(tss200) |
| cg13679303 | 96623674 | − | S_Shore | |
| cg22162848 | 73717736 | − | Island | ITGB4(body) |
| cg22079043 | 1785631 | + | S_Shore | CTSD(tss1500); MOB2(tss200) |
| cg25705148 | 72079498 | + | Island | |
| cg15154047 | 1.43E+08 | − | | |
| cg23013473 | 77881799 | − | | |
| cg23345697 | 520489 | − | S_Shelf | |
| cg17032564 | 1.59E+08 | − | Island | |
| cg20113012 | 71308660 | − | Island | CDC42EP4(tss1500) |
| cg17841545 | 1.15E+08 | − | | NRAP(body) |
| cg06917617 | 2.08E+08 | − | Island | CR1L(body) |
| cg00034755 | 50918361 | − | N_Shore | |
| cg22755142 | 16243377 | + | | |
| cg25453625 | 4347751 | + | Island | |
| cg06962684 | 34839039 | − | | CELF4(body) |
| cg06552810 | 31128660 | + | | |
| cg07041748 | 30023780 | − | S_Shore | DOC2A(body); DOC2A(tss1500); DOC2A |
| | | | | (tss200) |
| cg05725404 | 58534157 | − | N_Shore | NDRG4(body) |
| cg06901759 | 47001709 | − | Island | |
| cg22603450 | 551536 | − | Island | |
| cg14059257 | 52628105 | − | S_Shore | KRT7(body) |
| cg14013080 | 95826997 | − | S_Shore | |
| cg03077077 | 1.23E+08 | + | N_Shore | PP12613(tss1500); TMEM155(body) |
| cg14395744 | 72837693 | + | Island | |
| cg03895348 | 71062570 | + | | CD207(body) |
| cg06682185 | 61157615 | + | | SERPINB5(body) |
| cg03490759 | 1E+08 | − | Island | HHIPL1(body) |
| cg12669395 | 30860866 | − | | DDR1(body) |
| cg25314315 | 39407387 | + | | RHBDL2(body) |
| cg13825930 | 8751330 | − | Island | |
| cg10107890 | 44314289 | + | Island | |
| cg11975790 | 30853608 | − | S_Shore | DDR1(body) |
| cg10533103 | 2.13E+08 | + | S_Shore | |
| cg15638969 | 1.32E+08 | + | S_Shore | |
| cg01765164 | 84953424 | + | | |
| cg14308254 | 1.24E+08 | + | Island | FAM83A(body) |
| cg10096100 | 26503862 | − | | CNKSR1(tss200) |
| cg08275704 | 4588350 | + | S_Shelf | |
| cg09782034 | 61522112 | + | S_Shore | RORA(tss1500) |
| cg27305917 | 47396773 | − | S_Shelf | |
| cg19084479 | 44163941 | + | S_Shore | POLD2(tss1500) |
| cg08149865 | 21914600 | − | S_Shore | DMTN(body); DMTN(tss1500) |
| cg11344005 | 50987527 | + | Island | KLHDC78(body) |
| cg17055821 | 30669476 | + | S_Shore | C17orf75(tss1500) |
| cg02736908 | 29887376 | + | Island | |
| cg25136988 | 31649623 | + | N_Shore | LY6G5C(tss1500) |
| cg09404429 | 21367130 | + | Island | |
| cg07842521 | 85157762 | + | N_Shore | |
| cg12847793 | 30852874 | + | S_Shore | DDR1(body) |
| cg09649266 | 88583529 | − | Island | |
| cg23579185 | 77545223 | − | Island | |
| cg24870966 | 79282893 | + | N_Shore | LINC00482(body) |
| cg02718966 | 42846970 | + | S_Shore | HIGD1A(tss1500) |
| cg15883853 | 13080282 | + | | |
| cg12645284 | 79092878 | + | Island | ADAMTS7(body) |
| cg21514735 | 61325952 | + | | LOC100505532(body) |
| cg09622269 | 76803925 | + | S_Shore | DUPD1(body) |
| cg02389590 | 1.11E+08 | − | S_Shore | SYBU(body); SYBU(tss1500) |
| cg250S0332 | 316088 | − | Island | |
| cg07943780 | 41502612 | + | N_Shore | |
| cg17024542 | 1.43E+08 | + | N_Shore | CHST2(tss1500) |
| cg12850379 | 54935605 | − | S_Shore | TCEA1(tss1500) |
| cg13844049 | 68759259 | − | | |
| cg23904955 | 1.01E+08 | − | Island | |
| cg20372230 | 1.28E+08 | − | Island | |
| cg13879655 | 27450777 | + | S_Shore | |
| cg12887581 | 1.31E+08 | − | | |
| cg14071650 | 1.29E+08 | − | S_Shore | LMX1B(body) |
| cg27503950 | 1.6E+08 | − | | |
| cg05225012 | 96055346 | + | S_Shore | |
| cg06264679 | 32121433 | − | N_Shore | LOC100507547(body); PPT2(body); |
| | | | | PPT2(tss1500); PPT2- |
| | | | | EGFL8(tss1500) |
| cg23915527 | 1.61E+08 | + | N_Shore | |
| cg12565126 | 24233931 | − | S_Shore | MFSD2B(body) |
| cg22225479 | 25681463 | + | S_Shore | |
| cg14085177 | 1.33E+08 | − | S_Shelf | |
| cg08532057 | 25875436 | + | Island | NUPL1(tss1500) |
| cg15012468 | 2.06E+08 | + | S_Shore | MFSD4(body) |
| cg26580869 | 611506 | − | Island | HCN2(body) |
| cg26927427 | 57832230 | + | N_Shelf | KIFC3(body); KIFC3(tss1500) |
| cg23573129 | 19751148 | − | N_Shore | TBX1(body) |
| cg17395619 | 4607540 | − | S_Shore | |
| cg06660015 | 1.4E+08 | − | S_Shore | SLC4A9(body) |
| cg07647651 | 42878931 | + | N_Shore | |
| cg19019345 | 8888814 | + | N_Shelf | ST5(body) |
| cg10963061 | 1.58E+08 | + | Island | |
| cg18876602 | 17393472 | − | | CAP2(tss1500) |
| cg07140194 | 73150236 | + | N_Shore | EMX1(body) |
| cg06464775 | 1.51E+08 | − | Island | |
| cg09373148 | 1.38E+08 | + | N_Shore | ESYT3(tss1500) |
| cg05324273 | 60086738 | + | | |
| cg21980100 | 20799644 | − | Island | CCDC144NL(tss200); LOC440416(body) |
| cg07107628 | 27210934 | + | | |
| cg00071446 | 1.2E+08 | + | N_Shore | |
| cg06680906 | 69325560 | + | Island | MIR548H4(body); NOX5(body) |
| cg06971773 | 71169083 | + | S_Shore | TACR2(body) |
| cg01936707 | 30860905 | + | | DDR1(body) |
| cg22871908 | 1.11E+08 | − | Island | |
| cg24912456 | 1598854 | + | N_Shore | |
| cg06892912 | 30043858 | + | S_Shore | RNF39(tss1500) |
| cg06767142 | 63821400 | + | | MACROD1(body) |
| cg01422370 | 73384389 | + | | |
| cg05589046 | 13742034 | + | | LINC00620(body) |
| cg21327235 | 7643798 | − | Island | DNAH2(body) |
| cg08687941 | 57918350 | + | Island | CNGB1(body) |
| cg21158317 | 19314299 | − | | RNF112(tss200) |
| cg02695062 | 30851417 | − | N_Shore | DDR1(tss1500) |
| cg16698038 | 5495215 | − | Island | |
| cg22379862 | 1904502 | + | N_Shore | CACNA2D4(body) |
| cg26063721 | 34499488 | − | Island | PACSIN1(body) |
| cg18041814 | 36802896 | + | N_Shelf | CPNE5(body) |
| cg24309555 | 21266956 | + | Island | APOB(tss200) |
| cg12412902 | 34499504 | − | Island | PACSIN1(body) |
| cg27565473 | 47398007 | + | Island | |
| cg05975881 | 3148982 | − | | ZSCAN10(body) |
| cg11931463 | 1.57E+08 | + | S_Shore | LINC00880(body); LINC00881(tss1500) |
| cg10314139 | 71560231 | − | N_Shore | COL13A1(tss1500) |
| cg12967164 | 1753637 | + | Island | ELFN1(body) |
| cg17288288 | 51418092 | − | Island | DLEU7(tss1500); DLEU7-AS1(body) |
| cg02611848 | 74875387 | + | Island | M1AP(body); M1AP(tss1500) |
| cg05170706 | 1.13E+08 | + | | |
| cg20543681 | 642673 | − | Island | SCRT2(body) |
| cg01907837 | 37321337 | − | N_Shore | RNF8(tss1500) |
| cg17594670 | 2.1E+08 | + | | |
| cg25364822 | 33337257 | − | | FNDC5(body); FNDC5(tss1500) |
| cg21235746 | 5485144 | − | | LINC00654(body) |
| cg15022678 | 4877609 | + | S_Shore | RADIL(body) |
| cg19666541 | 31516111 | − | | LOC283710(body) |
| cg19419650 | 7929643 | + | S_Shore | EVISL(body) |
| cg26651775 | 764420 | + | N_Shore | METRN(tss1500) |
| cg20645966 | 1.17E+08 | + | | |
| cg05214460 | 1.12E+08 | + | N_Shore | ADD3(body); ADD3(tss1500); ADD3- |
| | | | | AS1(body) |
| cg10429573 | 2.41E+08 | + | | |
| cg15138109 | 24713234 | + | N_Shore | |
| cg13102294 | 32121393 | − | N_Shore | LOC100507547(body); PPT2(body); |
| | | | | PPT2(tss1500); PPT2- |
| | | | | EGFL8(tss1500) |
| cg13882377 | 1.26E+08 | + | | LHPP(body) |
| cg04726200 | 2930485 | + | Island | SLC22A18(body) |
| cg26292895 | 1.12E+08 | + | | RBM20(body) |
| cg04582871 | 63827695 | − | | MACROD1(body) |
| cg19981409 | 89225042 | − | S_Shore | NOX4(body); NOX4(tss1500) |
| cg24895155 | 1.28E+08 | − | | |
| cg07028396 | 93805682 | + | Island | |
| cg03469862 | 68924853 | − | Island | LOC338694(body) |
| cg13347577 | 30243828 | − | N_Shore | |
| cg15277677 | 1.32E+08 | − | | NTM (body) |
| cg13019868 | 1.29E+08 | − | Island | |
| cg12187687 | 27253834 | + | Island | |
| cg09859240 | 1.22E+08 | + | S_Shore | MCMBP(tss1500) |
| cg18676967 | 2.33E+08 | + | Island | |
| cg12468774 | 49236860 | − | Island | CCDC36(body); CCDC36(tss200) |
| cg27199697 | 48067475 | + | N_Shelf | DLX3(body) |
| cg14688272 | 80673958 | + | N_Shore | FN3KRP(tss1500) |
| cg13555278 | 26348484 | + | | EXTL1(body) |
| cg04264070 | 55887704 | + | N_Shore | TMEM190(tss1500) |
| cg23373640 | 65696480 | − | | |
| cg12222949 | 1.84E+08 | + | Island | COLGALT2(body) |
| cg04352168 | 86334058 | + | N_Shelf | |
| cg27260867 | 1.13E+08 | + | N_Shore | |
| cg11314274 | 1.32E+08 | + | | |
| cg18536118 | 14901012 | + | | |
| cg06621691 | 4175814 | + | | SDK1(body) |
| cg10158997 | 31734106 | − | | VWA7(body) |
| cg26388730 | 64405764 | + | Island | NRXN2(body) |
| cg14331765 | 1.22E+08 | − | | |
| cg18304280 | 1.03E+08 | + | N_Shore | |
| cg00873811 | 56654264 | + | S_Shelf | |
| cg14692950 | 7644073 | − | Island | DNAH2(body) |
| cg07603878 | 11773280 | + | Island | |
| cg12177762 | 1.27E+08 | + | | C3orf56(tss1500) |
| cg27206026 | 57907171 | + | | |
| cg11540997 | 45407818 | + | N_Shore | DUOX2(tss1500); DUOXA2(body) |
| cg03466522 | 16400754 | − | S_Shore | FAM131C(tss1500) |
| cg08125503 | 16119344 | − | Island | NCOR1(tss1500); PIGL(tss1500) |
| cg24620905 | 1.5E+08 | + | | MTMR11(body); MTMR11(tss1500) |
| cg15033140 | 1.7E+08 | − | | |
| cg06946880 | 71163123 | − | | ATP6V1B1(body) |
| cg09791504 | 1.52E+08 | + | | RIIAD1(body) |
| cg07637741 | 1.59E+08 | + | | SYTL3(body) |
| cg01695406 | 55889276 | − | Island | TMEM190(body) |
| cg05872387 | 2.43E+08 | − | | GAL3ST2(body) |
| cg03985801 | 2.02E+08 | + | N_Shore | LGR6(body); LGR6(tss1500) |
| cg17347335 | 93583973 | − | S_Shore | VSTM5(tss1500) |
| cg15069551 | 25128695 | − | N_Shore | LOC284798(body); LOC284798(tss200) |
| cg12245717 | 22555206 | − | N_Shelf | LINC00261(body) |
| cg19863595 | 96350605 | − | Island | AMDHD1(body) |
| cg01817364 | 43037411 | + | Island | |
| cg09982942 | 1.13E+08 | + | S_Shore | FBLN7(body) |
| cg17635777 | 1.42E+08 | + | | MROH5(body) |
| cg16675054 | 1.72E+08 | + | S_Shore | STK10(tss1500) |
| cg09011891 | 1.35E+08 | − | N_Shore | C5orf66(body) |
| cg17808631 | 1.02E+08 | − | S_Shore | SNX31(tss1500) |
| cg24172175 | 48988470 | − | Island | |
| cg12690246 | 50356869 | − | S_Shore | AQP5(body) |
| cg04729574 | 1.84E+08 | + | N_Shore | ECE2(body); ECE2(tss1500) |
| cg21124375 | 2.33E+08 | + | Island | |
| cg17466082 | 1557859 | − | | |
| cg13047869 | 10149882 | − | Island | FANCD2OS(body) |
| cg25570495 | 50419231 | − | S_Shore | SALL4(tss200) |
| cg15808604 | 74419428 | − | N_Shore | LOC283731(body) |
| cg23957311 | 1.47E+08 | + | | ZIC4(body) |
| cg25215834 | 78166267 | + | S_Shore | CARD14(body) |
| cg12559119 | 63590575 | − | | |
| cg02184697 | 3013025 | + | Island | KREMEN2(tss1500) |
| cg06086012 | 1.7E+08 | + | | CLDN11(body) |
| cg17024199 | 74881747 | + | S_Shelf | MGAT5B(body) |
| cg23940612 | 2.19E+08 | + | Island | RUFY4(tss1500) |
| cg10243004 | 1.29E+08 | − | S_Shore | POLR2D(tss1500) |
| cg06737907 | 66420062 | − | | MEGF11(body) |
| cg26906273 | 39547881 | − | Island | |
| cg19642408 | 9894335 | + | | |
| cg16670359 | 82542806 | + | Island | |
| cg18156486 | 2884295 | + | Island | |
| cg21460582 | 76005861 | + | | CSPG4(tss1500) |
| cg17600393 | 1051107 | + | N_Shore | SNTG2(body) |
| cg16276982 | 29968032 | − | S_Shore | |
| cg01602730 | 1.54E+08 | − | | |
| cg02951357 | 59280619 | + | N_Shore | LOC100131060(body) |
| cg27664689 | 1.53E+08 | + | | CACNB4(body); CACNB4(tss200) |
| cg09501821 | 67356387 | + | N_Shore | |
| cg10832281 | 35499741 | + | | PAMR1(body) |
| cg07513768 | 69744528 | − | N_Shore | RPLP1(tss1500) |
| cg04124909 | 1.82E+03 | − | | ZNF648(body) |
| cg17379860 | 61159602 | − | N_Shore | TMEM216(tss1500) |
| cg24566261 | 30854164 | + | S_Shore | DDR1(body) |
| cg06707910 | 17640812 | + | S_Shore | CECR5(body); CECR5(tss1500); CECR5- |
| | | | | AS1(body) |
| cg18786782 | 13936527 | − | Island | |
| cg10894991 | 2.01E+08 | + | | |
| cg22801949 | 1.81E+08 | + | N_Shore | IER5(tss1500) |
| cg03453916 | 31174468 | − | | |
| cg04755995 | 1.49E+08 | + | S_Shore | |
| cg07704990 | 1.02E+08 | + | Island | SLCO6A1(body) |
| cg09181015 | 1.58E+08 | − | | |
| cg05875017 | 2.33E+08 | + | S_Shore | CHRND(tss200) |
| cg12635602 | 58731357 | + | Island | LOC283194(body) |
| cg01850889 | 1.31E+08 | + | | |
| cg17718377 | 1.13E+08 | − | S_Shore | DTX1(tss200) |
| cg23226249 | 2928506 | + | N_Shore | |
| cg21764262 | 41473517 | + | S_Shore | |
| cg15142477 | 43915662 | − | S_Shore | RNF165(body) |
| cg12924302 | 1.54E+08 | + | Island | |
| cg07457305 | 71118292 | − | S_Shore | |
| cg20615832 | 74719172 | − | Island | PF4V1(body) |
| cg15132710 | 44237801 | − | S_Shore | LOXHD1(tss1500) |
| cg02849695 | 1.6E+08 | + | Island | CCDC19(tss200) |
| cg01569660 | 65369213 | − | Island | KBTBD13(body) |
| cg12012524 | 1268008 | − | Island | CIRBP(tss1500); CIRBP-AS1(body) |
| cg06951677 | 2.21E+08 | − | S_Shore | |
| cg06888460 | 18477303 | + | Island | LDHAL6A(tss200) |
| cg18480654 | 75376460 | + | N_Shore | |
| cg09894276 | 1.7E+08 | − | Island | WDR27(body) |
| cg17598923 | 1.01E+08 | − | | |
| cg02939598 | 11386395 | + | Island | |
| cg02635786 | 1.16E+08 | − | Island | TDRD1(body) |
| cg19395706 | 64412079 | − | S_Shore | NRXN2(body); NRXN2(tss1500) |
| cg11389868 | 35991624 | + | | UGT3A1(body); UGT3A1(tss200) |
| cg04760493 | 1.29E+08 | + | N_Shore | LARP1B(tss1500) |
| cg00091633 | 1.2E+08 | − | Island | FSTL1(tss1500) |
| cg01830154 | 1134911 | + | S_Shore | |
| cg04647654 | 53199284 | + | | |
| cg01057635 | 46914672 | + | S_Shore | |
| cg21206816 | 1.12E+08 | + | | SLC9C1(tss200) |
| cg00073549 | 87609078 | − | | |
| cg08537845 | 1.68E+08 | − | N_Shelf | |
| cg16707400 | 1416762 | − | | |
| cg21052814 | 79328213 | + | | RASGRF1(body) |
| cg23429749 | 1.02E+08 | − | Island | |
| cg20811236 | 46501400 | + | N_Shore | |
| cg23183559 | 1.21E+08 | − | | |
| cg18675610 | 32216311 | − | N_Shore | ARHGAP12(body) |
| cg05063520 | 49835999 | − | Island | |
| cg04452437 | 1.7E+08 | + | | |
| cg10500653 | 39407651 | − | | RHBDL2(tss200) |
| cg06081951 | 14991413 | − | N_Shelf | CALCA(body) |
| cg02625222 | 1.26E+08 | − | N_Shore | CRB2(body) |
| cg07138585 | 45176067 | + | N_Shore | |
| cg06881093 | 1.14E+08 | − | Island | PAX8(body) |
| cg02274869 | 11784955 | − | N_Shore | ZNF833P(body) |
| cg26955540 | 90727570 | − | N_Shore | SEMA4B(tss1500) |
| cg09246479 | 24891129 | − | Island | ADORA2A- |
| | | | | AS1(tss1500); UPB1(tss200) |
| cg25042226 | 1.14E+08 | − | S_Shore | PAX8(tss200) |
| cg13630250 | 70827336 | + | S_Shore | COX16(tss1500); SYNJ2BP- |
| | | | | COX16(body) |
| cg19225422 | 10971446 | + | | PDIA6(body) |
| cg06217323 | 75445502 | + | Island | |
| cg17385483 | 49734595 | − | N_Shore | |
| cg22392876 | 1.32E+08 | − | | |
| cg05863637 | 1.31E+08 | + | S_Shelf | |
| cg05902711 | 89114919 | − | S_Shore | |
| cg20545694 | 48910778 | + | | |
| cg11946630 | 99734081 | + | | CRTAC1(body) |
| cg24611264 | 1.2E+08 | + | S_Shore | TRIM29(tss200) |
| cg13476072 | 1.5E+08 | + | | MTMR11(body); MTMR11(tss200) |
| cg24780796 | 1.51E+08 | + | | SLC36A2(tss1500) |
| cg01512532 | 79495039 | − | Island | FSCN2(tss1500) |
| cg07776626 | 57350775 | + | N_Shore | |
| cg25511813 | 2717932 | − | S_Shore | DIRAS1(body) |
| cg01622718 | 1.58E+08 | + | S_Shore | PTPRN2(body) |
| cg27462791 | 1854368 | + | | SYT8(tss1500) |
| cg23735482 | 26222654 | − | N_Shore | MYO3A(tss1500) |
| cg16788656 | 87616174 | − | | |
| cg08203127 | 331727 | − | N_Shore | ARHGDIG(body); PDIA2(tss1500) |
| cg14448104 | 43933556 | + | N_Shore | MATN4(body) |
| cg01056004 | 98948259 | + | S_Shelf | ARHGAP19-SLIT1(body) |
| cg11316386 | 2.47E+08 | − | S_Shore | C1orf229(tss200) |
| cg15851904 | 64215680 | − | Island | |
| cg03918288 | 1.45E+08 | + | N_Shore | SPATC1(body) |
| cg09527118 | 58667960 | + | | |
| cg20806427 | 1.5E+08 | + | Island | |
| cg14543641 | 1.05E+08 | + | S_Shore | |
| cg06421590 | 3043752 | − | S_Shore | |
| cg07936030 | 36756043 | − | | SRCIN1(body) |
| cg07091678 | 99734120 | + | | CRTAC1(body) |
| cg22152492 | 28554334 | + | N_Shore | SCAND3(body) |
| cg26010879 | 73157217 | + | S_Shelf | |
| cg23281527 | 18809531 | − | Island | KLHDC7A(body) |
| cg22138302 | 1.19E+08 | − | N_Shore | ABCG4(tss1500) |
| cg26188543 | 1.49E+08 | + | | |
| cg26076054 | 421317 | + | Island | AHRR(body) |
| cg03350299 | 21266960 | + | Island | APOB(tss200) |
| cg10855395 | 1.25E+08 | − | Island | |
| cg20634798 | 1.53E+08 | − | | LCE3A(body) |
| cg24777351 | 1.66E+08 | − | | SLC38A11(body) |
| cg15602054 | 1.71E+08 | − | Island | |
| cg24849736 | 2.01E+08 | + | Island | ASCL5(body) |
| cg02975463 | 45178953 | + | N_Shore | |
| cg01018110 | 1.35E+08 | − | | |
| cg02087079 | 1.78E+08 | + | | COL23A1(body) |
| cg06019590 | 496476 | + | N_Shore | SLC9A3(body) |
| cg11523134 | 3605747 | + | N_Shore | TP73(body); TP73(tss1500) |
| cg11798891 | 1.43E+08 | + | Island | TSNARE1(tss1500) |
| cg24676346 | 41377288 | + | S_Shore | |
| cg13977620 | 1.2E+08 | + | | |
| cg21376799 | 30094960 | − | N_Shore | |
| cg17881353 | 44928210 | − | N_Shore | WNT9B(tss1500) |
| cg24006352 | 1.13E+08 | + | N_Shore | KCND3(body) |
| cg15575875 | 85220088 | − | Island | |
| cg11821245 | 18433683 | − | | LDHC(tss200) |
| cg05265884 | 26681244 | + | S_Shore | |
| cg23288838 | 23656074 | − | | |
| cg25351353 | 52473451 | − | Island | |
| cg03357547 | 51165207 | − | N_Shore | SHANK1(body) |
| cg01919208 | 49170496 | − | | LAMB2(body) |
| cg16655765 | 1.11E+08 | − | S_Shore | METTL24(tss1500) |
| cg08481491 | 1.26E+08 | − | S_Shore | ALDH1L1(tss1500); ALDH1L1(tss200); |
| | | | | ALDH1L1-AS2(body) |
| cg21415424 | 37503074 | + | N_Shore | |
| cg14688299 | 30728099 | + | | |
| cg10138352 | 64336120 | + | | |
| cg04456916 | 1.79E+08 | − | | |
| cg10061384 | 2466275 | + | N_Shore | |
| cg14581129 | 53358946 | − | N_Shore | |
| cg18358061 | 1.34E+08 | − | S_Shore | |
| cg05441133 | 48416891 | + | S_Shelf | GDF2(tss200) |
| cg08688548 | 83317398 | − | Island | CPEB1(tss1500); LOC283692(body) |
| cg20295248 | 5485270 | + | | LINC00654(tss200) |
| cg27430293 | 89069935 | + | N_Shore | |
| cg01890726 | 1.34E+08 | − | Island | GLB1L2(body) |
| cg08496033 | 1.44E+08 | + | | |
| cg15794873 | 1.27E+08 | − | | |
| cg02721176 | 1.18E+08 | − | | CCDC172(body) |
| cg27190145 | 39761304 | + | S_Shore | DAAM2(body) |
| cg02446487 | 49177504 | + | S_Shelf | NTN5(tss1500); SEC1P(body) |
| cg19594666 | 1.28E+08 | − | Island | LEP(tss200) |
| cg06872257 | 16859636 | + | S_Shore | FGF20(body) |
| cg07634019 | 1.39E+08 | − | N_Shore | PSD2(tss1500) |
| cg22539356 | 87667434 | − | | |
| cg18955367 | 49002338 | + | Island | LMTK3(body) |
| cg15890794 | 12258991 | + | S_Shelf | CIDEA(body) |
| cg08856033 | 1.21E+08 | − | Island | |
| cg00653790 | 78031418 | + | | |
| cg25753631 | 25732923 | + | Island | HI5T1H2APS1(body) |
| cg09015973 | 1.32E+08 | − | Island | ARHGEF4(tss1500) |
| cg02032841 | 23713275 | − | | |
| cg11001769 | 61748440 | + | S_Shore | TMEM30B(body) |
| cg11350504 | 1.13E+08 | + | N_Shore | |
| cg00161556 | 19138045 | − | N_Shore | ZDHHC13(tss1500) |
| cg22126965 | 1015501 | + | S_Shore | COX19(tss1500) |
| cg03400060 | 78365801 | − | S_Shore | BHMT2(body); DMGDH(tss1500) |
| cg09813050 | 1.29E+08 | + | Island | |
| cg04675946 | 26047805 | − | Island | |
| cg09656511 | 6292615 | + | Island | CCKBR(body) |
| cg23824713 | 61157738 | + | | SERPINB5(body) |
| cg01786585 | 1.55E+08 | − | N_Shore | |
| cg23083049 | 1.25E+08 | − | | KIRREL3(body) |
| cg13677022 | 34625963 | − | S_Shore | ARID3C(body) |
| cg11697038 | 27931284 | + | S_Shore | AHDCL(tss1500) |
| cg02959725 | 1.49E+08 | − | | |
| cg17394530 | 1.06E+08 | − | S_Shore | |
| cg07292311 | 1.26E+08 | + | | ZNF664-FAM101A(body) |
| cg20132698 | 86617261 | − | N_Shore | |
| cg01786048 | 2.39E+08 | − | N_Shore | ESPNL(body) |
| cg11684897 | 2951666 | + | N_Shore | |
| cg17481703 | 76444831 | − | | C13orf45(tss1500) |
| cg23635560 | 27473369 | + | | |
| cg13337662 | 25255297 | − | S_Shore | ATP12A(body) |
| cg03405128 | 77341841 | − | N_Shore | |
| cg13783444 | 75536434 | − | S_Shore | ZC2HC1C(body) |
| cg20354882 | 96350795 | − | S_Shore | AMDHD1(body) |
| cg08834436 | 27831818 | + | N_Shelf | |
| cg13708016 | 1.26E+08 | − | S_Shore | |
| cg16988970 | 42248062 | − | | ASB16(tss200) |
| cg00188627 | 1.6E−08 | − | | IQCJ-SCHIP1(body); SCHIP1(body) |
| cg01497154 | 3661910 | − | N_Shore | ADAM33(body) |
| cg22588640 | 95963179 | − | | KCNIP3(body) |
| cg20724680 | 49000743 | − | Island | LMTK3(body) |
| cg25486824 | 13983790 | − | N_Shore | SPON1(tss1500) |
| cg11316100 | 35233380 | − | N_Shore | C20orf24(tss1500); TGIF2- |
| | | | | C20orf24(body) |
| cg16592832 | 24891141 | − | Island | ADORA2A- |
| | | | | AS1(tss1500); UPB1(tss200) |
| cg02637101 | 99548944 | − | | PGPEP1L(body); PGPEP1L(tss200) |
| cg26682922 | 50880150 | − | N_Shore | |
| cg13982318 | 72131655 | − | Island | APBA1(body) |
| cg26333393 | 1.26E+08 | + | S_Shore | CPXM2(tss1500) |
| cg03731646 | 74499372 | + | Island | |
| cg16734433 | 1.16E+08 | − | N_Shore | |
| cg17035539 | 2.3E+08 | − | S_Shore | |
| cg18564464 | 46937902 | + | N_Shelf | |
| cg01706515 | 49681774 | − | | CRISP2(tss1500) |
| cg24332389 | 6558085 | + | S_Shore | PLEKKG5(body); PLEKHG5(tss1500) |
| cg27113419 | 58533979 | − | N_Shore | NDRG4(body); NDRG4(tss200) |
| cg23490796 | 2.2E+08 | − | N_Shore | DES(tss200) |
| cg00126498 | 1.5E+08 | + | S_Shore | ULBP1(body) |
| cg04189591 | 42309398 | − | N_Shore | SHISA8(body) |
| cg09477019 | 56681945 | − | | |
| cg15209808 | 30476254 | − | Island | HORMAD2(tss200) |
| cg11599830 | 49504967 | − | S_Shore | LMBR1L(tss1500) |
| cg00093178 | 1.13E+08 | + | N_Shore | |
| cg16406611 | 300404 | + | S_Shore | IFITM5(tss1500) |
| cg12946961 | 8870588 | + | | |
| cg23175533 | 13196876 | − | N_Shore | KIAA1467(tss1500) |
| cg10938046 | 43969577 | − | N_Shore | C6orf223(body) |
| cg17962756 | 1.73E+08 | − | | |
| cg04733365 | 18130049 | + | S_Shelf | |
| cg05309280 | 1.72E+08 | + | N_Shore | GORASP2(body) |
| cg26332285 | 1.35E+08 | − | S_Shore | GPR123(body) |
| cg16524232 | 88445933 | + | N_Shelf | |
| cg11241151 | 11123476 | + | | |
| cg24735129 | 70957520 | + | | |
| cg10350350 | 43983977 | + | | |
| cg26211724 | 57636670 | − | Island | |
| cg23669855 | 1E+08 | + | N_Shore | LYSMD4(body) |
| cg13736131 | 8832907 | + | | ST5(body); ST5(tss1500); ST5(tss200) |
| cg13924715 | 10750890 | + | | |
| cg06626847 | 1.49E+08 | + | Island | |
| cg23604683 | 75779470 | − | N_Shore | |
| cg05404005 | 8053420 | − | Island | |
| cg19628603 | 32847596 | − | Island | |
| C616598508 | 88228364 | − | | |
| cg14130459 | 60718568 | − | Island | SLC15A3(body) |
| cg22496437 | 17900032 | − | | ARHGEF10L(body) |
| cg08266168 | 1.14E+08 | − | N_Shore | F7(tss200) |
| cg16306259 | 1595099 | + | N_Shelf | |
| cg13443627 | 59043070 | + | Island | TACSTD2(body) |
| cg07337544 | 96013196 | + | Island | KCNIP3(body) |
| cg16555466 | 92042333 | − | S_Shore | |
| cg13810327 | 1.35E+08 | − | | C9orf171(body) |
| cg26740318 | 18477153 | + | N_Shore | LDHAL6A(tss1500) |
| cg19094438 | 75286009 | + | S_Shore | BCAR1(body); BCAR1(tss1500) |
| cg18449739 | 1.13E+08 | + | S_Shore | DTX1(body) |
| cg09777637 | 1.24E+08 | − | S_Shore | TMEM229A(tss1500) |
| cg17604312 | 30850829 | + | N_Shore | DDR1(tss1500) |
| cg04518186 | 77299962 | + | N_Shore | AQP11(tss1500) |
| cg14807565 | 94179972 | + | N_Shore | |
| cg20905088 | 28116451 | + | | PTHLH(body) |
| cg18621852 | 10150065 | + | Island | FANCD2OS(tss200) |
| cg03079132 | 727212 | − | N_Shelf | RHBDL1(body) |
| cg22625519 | 1524190 | Island | | |
| cg13099429 | 70827109 | + | S_Shore | COX16(tss1500); SYNJ2BP- |
| | | | | COX16(body) |
| cg24964694 | 36308389 | + | | |
| cg08276171 | 1.05E+08 | + | | |
| cg09122655 | 72027576 | − | | NPFFR1(body) |
| cg27455017 | 1.5E+08 | − | Island | RARRES2(body) |
| cg24940230 | 8046101 | − | | |
| cg14070755 | 2.38E+08 | + | N_Shore | MLPH (tss1500); MLPH(tss200) |
| cg09113440 | 18999313 | − | Island | |
| cg01294283 | 1.21E+08 | + | | |
| cg24848998 | 14567945 | + | | GRIP2(body) |
| cg05161119 | 785696 | + | N_Shore | CPLX1(body) |
| cg16292132 | 1.39E+08 | − | | NHSL1(body) |
| cg06906952 | 2.44E+08 | − | | |
| cg16449012 | 17781880 | + | N_Shore | FAM184B(body) |
| cg03717239 | 1.46E+08 | − | N_Shelf | |
| cg15146462 | 86142587 | − | Island | |
| cg23557906 | 467112 | + | N_Shore | |
| cg05225593 | 81437709 | + | S_Shore | |
| cg25478299 | 1.03E+08 | − | | |
| cg11402505 | 39523436 | + | S_Shore | FBXO27(tss1500) |
| cg01591830 | 89666000 | − | S_Shelf | |
| cg08681856 | 51411737 | + | Island | KLK4(body) |
| cg05100067 | 2295969 | − | | MYT1L(body) |
| cg01256227 | 10099432 | + | | |
| cg05697249 | 1.12E+08 | − | | C11orf52(body); HSPB2- |
| | | | | C11orf52(body) |
| cg12894629 | 1.96E+08 | + | | SLCS1A(tss1500) |
| cg19642433 | 64783703 | − | N_Shore | C12orf56(body) |
| cg03336167 | 2930995 | − | Island | SLC22A18(body) |
| cg27255656 | 4853437 | − | S_Shore | CSMD1(tss1500) |
| cg06649280 | 24438781 | − | | MYOM3(tss200) |
| cg09372728 | 56624886 | − | S_Shore | MT3(body) |
| cg12976582 | 50359583 | − | Island | HYAL2(body) |
| cg00228891 | 2.08E+08 | + | Island | CR1L(body) |
| cg09635954 | 29605624 | − | N_Shore | PRR15(body) |
| cg12554483 | 1.35E+08 | + | Island | VENTX(tss200) |
| cg15452204 | 1.5E+08 | − | Island | CDX1(body) |
| cg02524475 | 1.46E+08 | + | Island | HHIP(tss1500); HHIP-AS1(body) |
| cg11945297 | 1.03E+08 | + | | GRHL2(body) |
| cg22753661 | 79092743 | − | Island | ADAMTS7(body) |
| cg17223846 | 3480363 | − | N_Shore | C19orf77(body) |
| cg14243817 | 1.39E+08 | − | S_Shelf | |
| cg27664085 | 89157815 | + | | |
| cg15490784 | 1.26E+08 | − | N_Shore | KLF15(body) |
| cg02717339 | 97524767 | − | S_Shore | ANKRD39(tss1500) |
| cg10593400 | 86044176 | + | S_Shore | DDAH1(tss200) |
| cg06678242 | 93614758 | − | N_Shore | RGMA(body) |
| cg09240747 | 79465866 | + | Island | |
| cg25071744 | 21266995 | + | S_Shore | APOB(tss200) |
| cg21451816 | 1468848 | − | | |
| cg13739485 | 1.36E+08 | + | | |
| cg19025461 | 16163610 | − | N_Shore | FLJ37453(body) |
| cg01067216 | 1.02E−108 | + | Island | |
| cg10501602 | 1.51E+08 | + | Island | NOS3(body) |
| cg24439315 | 51067388 | − | N_Shore | LRRC4B(body) |
| cg04821993 | 73002468 | − | | |
| cg18773260 | 46685292 | + | Island | HOXB7(body) |
| cg10143507 | 1.19E+08 | + | N_Shore | |
| cg13750214 | 1302619 | + | Island | |
| cg20966270 | 1.11E+08 | − | N_Shore | KCNC4(tss1500) |
| cg15692360 | 6104267 | + | S_Shore | FERMT1(tss200) |
| cg05227350 | 1.3E+08 | + | S_Shore | ZBTB44(tss1500) |
| cg06255037 | 41634447 | − | Island | CHADL(body) |
| cg00616135 | 63413614 | + | N_Shore | LACTB(tss1500) |
| cg09997546 | 8931473 | − | N_Shore | AKIP1(tss1500); ST5(body) |
| cg20017761 | 25128807 | + | Island | LOC284798(body); LOC284798(tss200) |
| cg14836960 | 1.09E+08 | − | | DPPA4(tss200) |
| cg08474334 | 1.57E+08 | + | S_Shore | INSRR(body); NTRK1(body) |
| cg19313402 | 2.2E+08 | + | S_Shore | RNU5F- |
| | | | | 1(body); SLC30A10(tss1500) |
| cg26799250 | 6358415 | − | Island | PITPNM3(body) |
| cg11783602 | 55087084 | + | | |
| cg01550017 | 42924215 | − | Island | |
| cg09970967 | 73602752 | − | N_Shore | PSEN1(tss1500) |
| cg19572055 | 685830 | − | N_Shore | |
| cg26214645 | 80292081 | − | Island | SECTM1(tss200) |
| cg20129827 | 1.18E+08 | + | Island | KIAA1210(tss200) |
| cg25472530 | 23507617 | + | | PRDM9(tss200) |
| cg01030476 | 2.37E+08 | − | N_Shore | LGALS8(body); LGALS8(tss1500); LGALS8 |
| | | | | (tss200); LGALS8-AS1(body) |
| cg13260758 | 19736671 | + | | |
| cg08354681 | 4773278 | + | N_Shore | CDYL(body) |
| cg26904169 | 89839865 | − | N_Shore | STEAP2(tss1500) |
| cg02051924 | 11681482 | − | S_Shore | LITAF(tss1500); LITAF(tss200) |
| cg21710255 | 49681742 | + | | CRISP2(tss1500) |
| cg02734505 | 54763081 | − | Island | ZNF385A(body) |
| cg22109464 | 34543584 | − | | C21orf54(tss1500) |
| cg12352302 | 1.73E+08 | − | N_Shore | |
| cg01145903 | 1.16E+08 | + | Island | |
| cg06837568 | 1.09E+08 | + | | WSCD2(body) |
| cg00652772 | 1.03E+08 | − | Island | |
| cg08530064 | 1.51E+08 | + | | |
| cg13353050 | 32634406 | − | | |
| cg04467334 | 6291927 | − | N_Shore | CCKBR(body) |
| cg11590282 | 2036771 | − | S_Shelf | |
| cg06357843 | 50616150 | + | Island | PANX2(body) |
| cg15180789 | 1.6E+08 | + | | IQCJ-SCHIP1(body); SCHIP1(body) |
| cg15032861 | 2791584 | + | | |
| cg21117508 | 9887936 | − | S_Shelf | |
| cg15895197 | 27301490 | + | N_Shelf | EMILIN1(body) |
| cg12515485 | 1.33E+08 | + | N_Shore | LRCOL1(body) |
| cg04289036 | 43751270 | − | | C1orf210(body); C1orf210(tss200) |
| cg16530382 | 37123671 | + | | FBXO47(tss200) |
| cg08715146 | 46067137 | + | N_Shore | ATXN10(tss1500) |
| cg14793137 | 1480944 | + | Island | TPO(body) |
| cg04224786 | 1.44E+08 | − | | |
| cg05382497 | 3253789 | − | Island | MRGPRE(tss200) |
| cg04252915 | 1.21E+08 | − | N_Shore | |
| cg16450309 | 1.71E+08 | − | Island | EIF5A2(tss1500) |
| cg25918821 | 36811232 | − | | |
| cg00538923 | 74536011 | + | S_Shore | PRCD(body); PRCD(tss200) |
| cg05188837 | 87605249 | + | | |
| cg18771955 | 1.23E+08 | + | | |
| cg11540016 | 2978126 | − | | |
| cg05149097 | 59325534 | − | N_Shelf | |
| cg08206906 | 22701260 | + | Island | |
| cg26234644 | 10634427 | − | S_Shore | TMEM220(tss1500); TMEM220- |
| | | | | AS1(body) |
| cg25985959 | 1.06E+08 | + | S_Shore | |
| cg21459583 | 2.28E+08 | − | N_Shore | |
| cg00339913 | 22085227 | − | | PHYHIP(body) |
| cg07267550 | 74536269 | + | S_Shore | PRCD(body) |
| cg01689404 | 35423942 | + | | |
| cg09581049 | 42998320 | + | | |
| cg22670819 | 31913759 | − | | CFB(body) |
| cg08433293 | 89786622 | − | N_Shore | FANCI(tss1500) |
| cg24334149 | 71175539 | + | | ATP6V1B1(body) |
| cg15951661 | 71277141 | − | S_Shore | MAP3K9(tss1500) |
| cg10502231 | 2.24E+08 | − | | |
| cg05879418 | 54455955 | − | | CDC20B(body); GPX8(tss200) |
| cg14121987 | 37952644 | − | | |
| cg07147285 | 58282922 | − | N_Shore | CCDC113(tss1500) |
| cg10505973 | 1.05E+08 | − | Island | |
| cg04865726 | 1365911 | − | S_Shelf | |
| cg22697853 | 1.48E+08 | − | Island | NBPF8(body) |
| cg14051236 | 1E+08 | − | | CCDC85C(body) |
| cg20699305 | 18152403 | − | Island | ACTL8(body) |
| cg25714115 | 73895061 | − | Island | GTF2IRD1(body) |
| cg12904135 | 1.72E+08 | + | N_Shore | |
| cg20623503 | 78152219 | + | N_Shelf | CAR014(body); CARD14(tss200) |
| cg02582520 | 1009172 | + | Island | GRIN3B(body) |
| cg23659134 | 1.06E+08 | − | S_Shore | GSTO2(body) |
| cg15959252 | 48578724 | − | S_Shore | C12orf68(body) |
| cg01644795 | 11617305 | − | | C8orf49(tss1500); GATA4(body) |
| cg03716984 | 54538505 | − | | |
| cg11604426 | 30592406 | + | N_Shore | RHBDL3(tss1500) |
| cg01905210 | 1.38E+08 | + | Island | ESYT3(body) |
| cg14844953 | 19339051 | − | | |
| cg00012362 | 1.54E+08 | − | | S100A1(tss1500); S100A13(body) |
| cg10907148 | 72948349 | + | | HID1(body) |
| cg02303258 | 1.35E+08 | + | N_Shore | |
| cg13886554 | 1.13E+08 | + | N_Shore | FBLN7(tss1500) |
| cg00962755 | 2.01E+08 | + | S_Shore | PHLDA3(tss1500) |
| cg25740457 | 6292896 | + | S_Shore | CCKBR(body) |
| cg03044367 | 22581534 | + | | |
| cg17641252 | 67878535 | + | Island | PLEK2(body) |
| cg13491480 | 1.31E+08 | + | | LOC100289019(body) |
| cg24854181 | 6823241 | + | Island | |
| cg20356482 | 97356092 | − | | FBP2(body) |
| cg19317226 | 1.31E+08 | − | | |
| cg05124243 | 22767076 | + | S_Shore | |
| cg26929272 | 88295210 | − | Island | |
| cg16627090 | 44105050 | + | Island | PGAM2(body) |
| cg21936179 | 2.07E+08 | − | S_Shore | EIF2D(tss1500) |
| cg06789686 | 42705761 | − | | |
| cg27239981 | 1.66E+08 | + | N_Shelf | |
| cg22953960 | 22766081 | + | Island | |
| cg07472579 | 10660390 | + | | |
| cg19925872 | 90543392 | − | N_Shore | ZNF710(tss1500) |
| cg04582672 | 97127338 | − | | |
| cg03856044 | 1.6E+08 | − | | APCS(body) |
| cg26878893 | 1.11E+08 | − | S_Shore | ALX3(tss1500) |
| cg02852670 | 20903410 | + | Island | KLHL33(body) |
| cg14087574 | 1963794 | + | S_Shore | |
| cg05657709 | 1.22E+08 | − | | ILDR1(tss1500) |
| cg16044810 | 1.51E+08 | − | S_Shore | CRYGN(tss1500) |
| cg13952899 | 2.27E+08 | + | S_Shore | C1orf95(body) |
| cg06483432 | 55523233 | − | N_Shore | KIT(tss1500) |
| cg00404187 | 1.4E+08 | + | Island | |
| cg15399533 | 1.08E+08 | + | Island | NR2E1(body) |
| cg07278425 | 27137922 | + | S_Shore | HOTAIRM1(body) |
| cg16277071 | 75537317 | − | | |
| cg18888461 | 18477379 | + | Island | LDHAL6A(body) |
| cg21144120 | 28661199 | − | | |
| cg20378690 | 22723551 | + | Island | PEBP4(body) |
| cg23973272 | 52322169 | + | Island | PXDNL(body) |
| cg12905571 | 1.83E+08 | − | | |
| cg10910512 | 1.13E+08 | − | S_Shore | LAMA4(tss200) |
| cg10186366 | 71898817 | + | S_Shore | |
| cg14644418 | 1.3E+08 | + | Island | FOXI2(body) |
| cg05266836 | 55124187 | − | S_Shore | |
| cg08363684 | 761547 | − | | |
| cg02008416 | 55445880 | − | N_Shore | TMEM61(tss1500) |
| cg23948204 | 1.46E+08 | − | Island | |
| cg11183181 | 5421214 | − | N_Shore | |
| cg01692252 | 37893005 | + | | GRB7(tss1500) |
| cg25976170 | 29707585 | + | Island | GAS2L1(body) |
| cg18432895 | 29205555 | + | N_Shore | DUSP4(body) |
| cg05994850 | 30095341 | + | Island | |
| cg00110832 | 32121130 | − | N_Shore | LOC100507547(body); PPT2(tss1500); |
| | | | | PPT2(tss200); PPT2- |
| | | | | EGFL8(tss1500); PRRT1(tss1500) |
| cg08638582 | 2717615 | − | Island | DIRAS1(body) |
| cg22285914 | 53729659 | − | | SP7(body); SP7(tss200) |
| cg04857403 | 43473030 | − | Island | |
| cg26410635 | 96350720 | + | Island | AMDHD1(body) |
| cg04387820 | 18999425 | + | S_Shore | |
| cg15458577 | 46947998 | − | N_Shelf | |
| cg17480077 | 10895517 | + | N_Shore | |
| cg08032971 | 46972736 | + | | DMBX1(body) |
| cg14195249 | 13571710 | − | S_Shore | |
| cg24977886 | 42470101 | + | Island | FAM109B(tss200) |
| cg02624051 | 42371635 | − | Island | PLA2G4D(body) |
| cg13993183 | 1.35E+08 | − | S_Shore | |
| cg27487796 | 28973253 | − | | |
| cg11194613 | 70268728 | − | | CTTN(body) |
| cg27649348 | 42944030 | + | N_Shore | |
| cg07889505 | 42349787 | − | | |
| cg00491927 | 1.2E+08 | − | N_Shore | |
| cg19655177 | 1.55E+08 | + | | |
| cg19021383 | 45922184 | − | N_Shore | MAPK8IP1(body) |
| cg02596940 | 3832362 | + | | |
| cg18461362 | 47866784 | + | S_Shore | |
| cg02072875 | 2606796 | − | | IQCE(body) |
| cg21171339 | 79791169 | − | N_Shore | VPS13A(tss1500) |
| cg15429214 | 43166281 | − | Island | |
| cg19691659 | 64482332 | − | S_Shore | NRXN2(body) |
| cg05181941 | 1.31E+08 | + | N_Shore | |
| cg09728985 | 53543985 | − | | |
| cg10654546 | 56617576 | − | N_Shore | NABP2(tss1500) |
| cg23464360 | 1.45E+08 | − | S_Shore | MAFA(tss1500) |
| cg13207534 | 5087447 | − | S_Shore | PPP1R3G(body) |
| cg09580336 | 1.17E+08 | − | N_Shore | ATP1A1(tss1500) |
| cg24001246 | 21949481 | + | S_Shore | RAP1GAP(body) |
| cg24264254 | 1.01E+08 | − | | |
| cg27158481 | 25128681 | − | N_Shore | LOC284798(body); LOC284798(tss200) |
| cg24308847 | 34653477 | − | Island | |
| cg18363035 | 20805385 | + | N_Shore | GJB6(body); GJB6(tss200) |
| cg16903060 | 24621673 | − | | |
| cg04440361 | 77895991 | − | | |
| cg02964065 | 32729545 | + | Island | HLA-DQB2(body) |
| cg19091930 | 191127 | − | N_Shore | LRRC14B(tss1500) |
| cg13598010 | 72838776 | + | Island | |
| cg10432422 | 17475819 | − | Island | |
| cg07511721 | 5335697 | + | N_Shore | SLC29A4(body) |
| cg22956649 | 1.77E+08 | + | S_Shelf | HOXD-AS1(body) |
| cg01109337 | 56744332 | − | | TEX14(body) |
| cg04690110 | 26556829 | + | | |
| cg16392860 | 34114384 | − | S_Shore | GRM4(body); GRM4(tss1500) |
| cg07545858 | 88482626 | + | | |
| cg25969122 | 1.2E+08 | + | | |
| cg27222736 | 1617377 | + | N_Shore | |
| cg18083764 | 4230848 | − | | |
| cg21745164 | 23765304 | + | N_Shore | CHP2(tss1500) |
| cg09562655 | 1.86E+08 | − | | |
| cg07547765 | 46685448 | + | Island | HOXB7(body) |
| cg19846667 | 22191856 | − | | |
| cg13503773 | 91605494 | − | N_Shore | C9orf47(tss1500); S1PR3(tss1500) |
| cg18303437 | 1.37E+08 | + | | |
| cg08084655 | 72358213 | − | S_Shore | BTBD17(tss1500) |
| cg01360281 | 1.54E+08 | + | Island | |
| cg05194362 | 1.14E+08 | − | S_Shore | PAX8(body) |
| cg00965023 | 11785188 | + | Island | ZNF833P(body) |
| cg12353636 | 85974086 | + | | CDHR1(body) |
| cg17846127 | 89940505 | − | N_Shelf | LINC00925(body) |
| cg21926804 | 10555382 | − | | C8orf74(body) |
| cg20883730 | 7052540 | + | | NLRP14(body) |
| cg08354406 | 1.15E+08 | − | | NRAP(body) |
| cg25851313 | 16400665 | − | S_Shore | FAM131C(tss1500) |
| cg21215218 | 19011172 | − | | PAX7(body) |
| cg15420683 | 44244395 | + | Island | TMEM151B(body) |
| cg13478289 | 33132650 | + | S_Shelf | COL11A2(body) |
| cg04452432 | 57406511 | + | | CX3CL1(body) |
| cg04939302 | 30213458 | + | | |
| cg26567736 | 1.32E+08 | − | | NTM(body) |
| cg20185617 | 1099651 | + | Island | |
| cg03550129 | 1596054 | + | S_Shore | |
| cg22683036 | 63249209 | + | | ATG4C(tss1500) |
| cg08898775 | 59042684 | − | S_Shore | ADAM10(tss1500) |
| cg13688889 | 1.01E+08 | + | N_Shore | |
| cg16555417 | 40598772 | − | Island | |
| cg25290227 | 1.14E+08 | + | S_Shore | GPAM(tss1500) |
| cg17512539 | 29404724 | + | | |
| cg21345949 | 1.35E+08 | − | Island | FUOM(tss200) |
| cg21436572 | 70623422 | + | | SLCO5A1(body) |
| cg22433862 | 28013341 | − | | |
| cg08590069 | 1.01E+08 | + | S_Shore | RGS22(tss1500) |
| cg19034028 | 68677364 | + | N_Shore | CDH3(tss1500) |
| cg23239574 | 2.29E+08 | − | S_Shore | |
| cg12134633 | 32934004 | − | | SCG5(body) |
| cg22678073 | 8087421 | + | S_Shore | ERRFI1(tss1500) |
| cg05316627 | 87861261 | − | N_Shore | |
| cg09581060 | 928673 | − | | |
| cg20059012 | 53613154 | + | N_Shore | RARG(body) |
| cg25194612 | 1.35E+08 | + | Island | |
| cg13015534 | 74639793 | − | | ST6GALNAC1(body) |
| cg05504055 | 46875704 | + | Island | PRSS42(tss200) |
| cg23800169 | 1.25E+08 | + | | |
| cg13430464 | 74408131 | − | | CHRDL2(body) |
| cg02621357 | 11296980 | + | | FAM167A(body) |
| cg11084470 | 35108061 | − | N_Shore | TCP11(body) |
| cg18815398 | 61506981 | − | N_Shore | |
| cg20693690 | 85442686 | − | | TCF7L1(body) |
| cg13754720 | 98274853 | − | S_Shore | PTCH1(body) |
| cg10613381 | 24890330 | − | N_Shore | ADORA2A- |
| | | | | AS1(body); UPB1(tss1500) |
| cg07651720 | 1.21E+08 | − | | |
| cg00943382 | 1.57E+08 | + | | |
| cg13972661 | 99776416 | − | S_Shore | HIATL2(tss1500) |
| cg10622551 | 2366555 | − | Island | ZFYVE28(body) |
| cg04493143 | 1.48E+08 | − | | HTR4(body) |
| cg04986555 | 55446708 | + | Island | TMEM61(body) |
| cg00798317 | 48153934 | + | | ITGA3(body) |
| cg02008818 | 1.71E+08 | − | N_Shore | EIF5A2(body) |
| cg27238470 | 41950325 | + | | EDN2(body) |
| cg23992493 | 21220479 | + | S_Shore | |
| cg04810284 | 79971177 | + | | LINC01088(body) |
| cg13792913 | 1.18E+08 | − | S_Shelf | |
| cg16186131 | 1.72E+08 | − | | |
| cg26995224 | 1.22E+08 | − | N_Shore | KDM2B(body) |
| cg10483275 | 1.5E+08 | + | | CAMK2A(body) |
| cg01891818 | 73008509 | − | N_Shore | ICT1(tss1500) |
| cg05465347 | 281115 | + | Island | |
| cg15560801 | 46918254 | + | S_Shelf | |
| cg14038058 | 1.66E+08 | − | | TRIM61(tss200) |
| cg19615684 | 6310744 | + | S_Shore | CD9(body) |
| cg16936370 | 56237925 | + | | MMP19(tss1500) |
| cg00407546 | 8094860 | − | Island | GATA3-AS1(body); GATA3- |
| | | | | AS1(tss1500) |
| cg07548255 | 1.1E+08 | + | Island | SH3RF3(body); SH3RF3-AS1(tss200) |
| cg01461856 | 19628012 | + | Island | |
| cg08355456 | 67383691 | − | Island | DOC2GP(tss1500) |
| cg08119295 | 1.03E+08 | + | | TCEAL7(body) |
| cg26843498 | 2361574 | − | S_Shore | |
| cg10556999 | 1.58E+08 | − | | |
| cg18795786 | 41087584 | − | S_Shore | CNTN1(body) |
| cg16137147 | 2.21E+08 | − | Island | |
| cg04089788 | 50738890 | + | Island | PLXNB2(body) |
| cg27584013 | 23012439 | + | | |
| cg18476766 | 2.41E+08 | − | | |
| cg25305153 | 2.37E+08 | + | | ASB18(body) |
| cg22147968 | 1.12E+08 | + | Island | CUX2(body) |
| cg19420604 | 60828406 | + | | MARCR10(body); MIR548W(body) |
| cg19445044 | 52596367 | + | | |
| cg14997592 | 49681178 | + | | CRISP2(body) |
| cg19254328 | 1.26E+08 | + | N_Shore | CCDC37(tss1500); LOC100506907(body) |
| cg08168768 | 1.18E+08 | + | Island | KIAA1210(tss1500) |
| cg13040392 | 1.36E+08 | − | Island | |
| cg05530317 | 38445774 | + | | EGFLAM(body) |
| cg19699056 | 73843743 | − | | |
| cg21408915 | 69286231 | − | Island | |
| cg20278854 | 1.96E+08 | − | | ZDHHC19(body) |
| cg25958766 | 5485268 | − | | LINC00654(tss200) |
| cg17347634 | 65711916 | + | S_Shore | CYP7B1(tss1500) |
| cg07477034 | 53341969 | + | N_Shore | HLF(tss1500) |
| cg25629768 | 2428567 | + | Island | LMNB2(body); TIMM13(tss1500) |
| cg02162144 | 1.02E+08 | − | | |
| cg15906733 | 35423405 | − | | |
| cg17285709 | 2.2E+08 | + | Island | SPEG(body) |
| cg05714155 | 1.73E+08 | − | S_Shore | |
| cg09289148 | 1480869 | − | Island | |
| cg22094845 | 2.33E+08 | + | S_Shore | ALPI(body) |
| cg05063952 | 37380697 | + | N_Shore | STAC2(body) |
| cg17971837 | 37215995 | − | S_Shelf | PVALB(tss1500) |
| cg20265528 | 1.06E+08 | − | Island | |
| cg15772924 | 54413384 | + | S_Shore | HOXC4(body); HOXC5(body); HOXC6 |
| | | | | (body) |
| cg04259907 | 43969884 | + | S_Shore | LYPD3(tss200) |
| cg24810305 | 1.03E+08 | − | Island | AMN(body) |
| cg25641533 | 31734580 | + | | VWA7(body) |
| cg01565291 | 2.46E+08 | + | | KIF26B(body) |
| cg00786761 | 1.84E+08 | − | Island | |
| cg19311244 | 77341912 | − | N_Shore | |
| cg09630142 | 31733889 | − | | VWA7(body) |
| cg18472410 | 42756786 | − | | CCDC152(tss200) |
| cg24485927 | 795989 | + | | LINC01115(body) |
| cg02537838 | 61002595 | + | | RBBP8NL(body) |
| cg17335199 | 6078738 | − | | LINC01115(body) |
| cg06494185 | 32992623 | − | | |
| cg21032567 | 795935 | + | | LINC01115(body) |
| cg26312542 | 1.12E+08 | + | | TEX12(body) |
| cg26276327 | 2.48E+08 | + | | |
| cg26932623 | 2.02E+08 | + | | |
| cg27592309 | 55326700 | + | | |
| cg13449377 | 96631089 | + | N_Shore | DLX6-AS1(body) |
| cg14827832 | 2454989 | − | | |
| cg13238680 | 65733273 | − | Island | |
| cg00765765 | 1.5E+08 | − | N_Shore | |
| cg15924332 | 7710179 | + | | ITIHS(tss1500) |
| cg20272155 | 1769462 | − | Island | IFITM10(body); MOB2(body) |
| cg21672550 | 34576199 | + | N_Shore | CNTFR(body); CNTFR-AS1-(body) |
| cg26719062 | 30095349 | + | Island | |
| cg24058386 | 1.56E+08 | − | S_Shelf | C1orf61(body) |
| cg04476394 | 1641647 | + | N_Shelf | |
| cg08951051 | 4993543 | − | | |
| cg12453539 | 6033866 | + | S_Shore | LRRN4(body) |
| cg23516451 | 38845028 | − | N_Shore | OSMR(tss1500) |
| cg11695684 | 2.33E+08 | + | S_Shelf | CHRND(body) |
| cg01824138 | 10699604 | − | Island | CASZ1(body) |
| cg14540913 | 1.32E+08 | − | N_Shore | PRRX2(body) |
| cg03071221 | 64176842 | + | | |
| cg20335213 | 94704048 | − | S_Shore | ARHGAP29(tss1500) |
| cg06289725 | 62689081 | + | N_Shore | CHRM1(tss200) |
| cg17596731 | 1726243 | + | Island | |
| cg00586527 | 1.3E+08 | + | Island | |
| cg24082680 | 63249199 | + | | ATG4C(tss1500) |
| cg11412129 | 88928839 | + | S_Shore | |
| cg03213289 | 61660250 | − | Island | LOC63930(body) |
| cg20016673 | 1.08E+08 | − | Island | NTNG1(body) |
| cg24743423 | 8701002 | + | S_Shore | KAL1(tss1500) |
| cg02837600 | 46714458 | − | N_Shore | LOC642852(body) |
| cg23948843 | 1.64E+08 | − | Island | |
| cg25521481 | 43214396 | + | Island | TTBK1(body) |
| cg06687091 | 43912991 | − | N_Shore | RNF165(tss1500) |
| cg24733126 | 9112214 | − | N_Shore | MIR5691(tss1500); SCUBE2(body) |
| cg00645383 | 1.13E+08 | − | Island | |
| cg17112828 | 2.05E+08 | − | S_Shore | LRRN2(tss1500) |
| cg11757417 | 98424289 | − | | |
| cg04193820 | 29297414 | + | N_Shore | RNF135(tss1500) |
| cg03720745 | 22561445 | − | S_Shore | |
| cg15687395 | 79379309 | + | | |
| cg26317834 | 4486136 | − | N_Shore | SMTNL2(tss1500) |
| cg11133013 | 33880214 | + | Island | FAM83C(body) |
| cg13921251 | 35036078 | + | | |
| cg27005487 | 59484388 | − | N_Shore | TBX2(body) |
| cg24856064 | 31506963 | − | | TEX26(body); TEX26-AS1(tss1500) |
| cg23422156 | 1.14E+08 | − | N_Shelf | CCDC42B(tss200) |
| cg24827726 | 14581937 | + | | GRIP2(body) |
| cg22291359 | 16119456 | − | Island | NCOR1(tss1500); PIGL(tss1500) |
| cg08636191 | 54900829 | − | Island | |
| cg08072539 | 496618 | − | S_Shore | TDRP(tss1500) |
| cg07903023 | 2.39E+08 | − | Island | KLHL30(body) |
| cg26422151 | 88136949 | − | Island | |
| cg08102414 | 1570791 | − | Island | |
| cg05622003 | 71234940 | + | | |
| cg02692893 | 1.24E+08 | + | N_Shelf | ROPN1(body) |
| cg03054684 | 39543515 | − | N_Shore | MOBP(body); MOBP(tss200) |
| cg01381795 | 54496611 | + | Island | CACNG6(body) |
| cg19537719 | 41583498 | + | Island | ANK1(body) |
| cg20265748 | 89017615 | − | N_Shore | PTPN21(body) |
| cg13698883 | 38422614 | + | N_Shore | IGFBPL1(body) |
| cg15712226 | 96252056 | − | Island | SNRPF(tss1500) |
| cg13839439 | 54144521 | + | N_Shore | CISTR-ACT(body) |
| cg13726191 | 15940033 | − | | FGFBP1(body) |
| cg06324373 | 99734805 | − | | CRTAC1(body) |
| cg14635586 | 91321898 | − | S_Shore | |
| cg26478107 | 26595280 | + | N_Shore | |
| cg02455471 | 77146197 | + | N_Shore | |
| cg25201363 | 3534845 | − | S_Shore | LRPAP1(tss1500) |
| cg21686577 | 75909905 | + | Island | SRRM3(body) |
| cg23173688 | 86081894 | − | Island | |
| cg16611278 | 1.14E+08 | + | N_Shore | DTX1(body) |
| cg00118978 | 14597418 | + | Island | |
| cg07009504 | 25115530 | + | N_Shore | |
| cg15768933 | 2.33E+08 | + | S_Shore | ECEL1(tss1500) |
| cg09322513 | 65555681 | − | | LINC00922(body) |
| cg00926894 | 46938229 | + | S_Shore | RGN(body) |
| cg16625671 | 1.58E+08 | − | | |
| cg02475474 | 29635158 | + | | MOG(body) |
| cg13912311 | 1.27E+08 | + | Island | NR5A1(body) |
| cg18244845 | 6111541 | + | S_Shore | RFX2(tss1500) |
| cg11421509 | 1.05E+08 | − | Island | |
| cg25397029 | 99338240 | + | Island | ANKRD2(body) |
| cg22130008 | 1.56E+08 | + | S_Shore | |
| cg06530441 | 51016950 | − | Island | CHKB- |
| | | | | CPT1B(body); CPT1B(body); CPT1B |
| | | | | (tss1500); CPT1B(tss200) |
| cg26550330 | 2.28E+08 | − | N_Shelf | C1orf145(body); OBSCN(body) |
| cg13870426 | 30244630 | − | S_Shore | |
| cg04090347 | 44061597 | − | | |
| cg19813226 | 2.35E+08 | + | | |
| cg16263848 | 9018806 | + | N_Shore | NTN1(body) |
| cg16985708 | 73247674 | + | S_Shore | |
| cg07253829 | 1.09E+08 | − | | DPPA4(body) |
| cg24933709 | 1.49E+08 | + | S_Shore | |
| cg15161667 | 46940613 | − | S_Shore | LRP4(tss1500) |
| cg11737172 | 1923052 | + | S_Shore | KBTBD11(body) |
| cg18569141 | 90727995 | − | Island | SEMA4B(tss200) |
| cg27364650 | 43899898 | + | | |
| cg08182193 | 1.06E+08 | + | S_Shore | CRIP2(body); CRIP2(tss1500) |
| cg17396305 | 1594275 | − | | |
| cg08178168 | 35934864 | − | N_Shore | |
| cg02116131 | 1175360 | + | S_Shore | |
| cg26582789 | 19531688 | + | N_Shore | MAP3K15(body) |
| cg05068206 | 30095195 | − | Island | |
| cg21293216 | 24469768 | − | | IL22RA1(body) |
| cg14325661 | 49699881 | − | Island | TRPM4(body) |
| cg11143671 | 1.81E+08 | + | N_Shore | KIAA1614(body) |
| cg19227924 | 28565709 | − | S_Shelf | |
| cg14413050 | 22615145 | + | | |
| cg07552868 | 1.45E+08 | − | | LOC100288142(body); NBPF9(body); |
| | | | | PDE4DIP(body) |
| cg02802029 | 1.46E+08 | − | S_Shore | PLOD2(tss1500) |
| cg05854695 | 1.23E+08 | + | S_Shore | TRPC3(body); TRPC3(tss200) |
| cg08784911 | 2.04E+08 | + | Island | |
| cg26416971 | 80529100 | + | | CKMT2(tss200); RNU5D- |
| | | | | 1(body); RNU5E-1(body) |
| cg02467858 | 99790901 | + | N_Shore | LRRC28(tss1500); TTC23(tss1500) |
| cg10130718 | 53925368 | − | Island | DMRTB1(body) |
| cg00019841 | 48256146 | + | Island | MAPK4(body) |
| cg21944224 | 1.84E+08 | − | Island | |
| cg23534245 | 6751435 | + | S_Shore | BMP2(body) |
| cg13096592 | 1.44E+08 | − | | |
| cg01244934 | 1003417 | + | Island | |
| cg14218621 | 1.35E+08 | − | | C9orf171(body) |
| cg14645861 | 30094987 | − | N_Shore | |
| cg13627197 | 25160378 | − | S_Shore | PIWIL3(body); TOP1P2(tss200) |
| cg24932585 | 12665317 | − | Island | |
| cg12927641 | 1.1E+08 | − | Island | |
| cg02737384 | 45470199 | − | Island | RASSF4(body) |
| cg18973515 | 65713476 | − | N_Shore | IGDCC4(body) |
| cg15676707 | 1.06E+08 | + | Island | |
| cg07733098 | 64478343 | − | N_Shore | NRXN2(body) |
| cg00738772 | 1.49E+08 | + | | |
| cg26057784 | 73362214 | − | S_Shore | DPF3(tss1500) |
| cg11191337 | 44541804 | − | S_Shore | PLTP(tss1500) |
| cg04661709 | 1.24E+08 | − | N_Shore | DAB2IP(body) |
| cg18279134 | 16394568 | + | | FAM131C(body) |
| cg12284521 | 34113943 | + | Island | GRM4(body); GRM4(tss200) |
| cg14283082 | 1.44E+08 | − | | |
| cg27118929 | 69325271 | − | N_Shore | MIR548H4(body); NOX5(body) |
| cg00788760 | 44682494 | + | | XIAA1644(body) |
| cg04396112 | 2151007 | − | N_Shelf | MAD1L1(body) |
| cg04898026 | 1.49E+08 | + | Island | SSPO(body) |
| cg15555527 | 80885981 | − | Island | ANTXR2(body) |
| cg10776186 | 25875020 | + | Island | NUPL1(tss1500) |
| cg27655214 | 95840848 | − | | |
| cg26316885 | 10407317 | + | Island | ICAM5(body) |
| cg12070646 | 71149811 | + | | VAX2(body) |
| cg10399005 | 70316898 | + | Island | |
| cg22219795 | 1.27E+08 | − | | C3orf56(tss1500) |
| cg18833933 | 1.33E+08 | + | N_Shore | LRCOL1(body) |
| cg27038634 | 49468341 | − | N_Shore | |
| cg02375506 | 43806801 | − | N_Shore | MPPED1(tss1500) |
| cg06219995 | 16374768 | + | | CLCNKB(body); CLCNKB(tss1500) |
| cg24408057 | 4277333 | + | N_Shore | SHD(tss1500) |
| cg22062555 | 45664251 | + | | NKPD1(tss1500) |
| cg20547777 | 1.19E+08 | + | | EXT1(body) |
| cg09049251 | 50297477 | − | N_Shore | FAIM2(body) |
| cg17529386 | 1.7E+08 | − | Island | |
| cg15645685 | 19730175 | − | S_Shore | PBX4(tss1500) |
| cg05298458 | 19749188 | + | Island | TBX1(body) |
| cg05973455 | 1.86E+08 | − | Island | |
| cg17841765 | 22560599 | + | | |
| cg27128734 | 51411940 | + | Island | KLK4(body) |
| cg15169829 | 51236693 | + | Island | TMPRSS12(tss200) |
| cg25569480 | 48844892 | + | Island | GTF2A1L(tss200); STON1- |
| | | | | GTF2A1L(body) |
| cg14126466 | 1.15E+08 | − | N_Shelf | TBX5(body) |
| cg21506565 | 31691505 | + | Island | C6orf25(body) |
| cg03177666 | 54365103 | − | | |
| cg13808198 | 8087518 | + | S_Shore | ERRFI1(tss1500) |
| cg05473289 | 32016257 | + | | TNXB(body) |
| cg01751047 | 89258141 | − | Island | CDH15(body) |
| cg06600725 | 48690915 | + | | |
| cg08853304 | 1.72E+08 | − | | |
| cg13683667 | 9527208 | − | | |
| cg05339633 | 1.84E+08 | + | N_Shore | MAP6D1(body) |
| cg06179011 | 843897 | + | S_Shore | POLR2L(tss1500); TSPAN4(body); TSPAN4 |
| | | | | (tss1500); TSPAN4(tss200) |
| cg06961278 | 1.14E+08 | + | N_Shore | |
| cg25447164 | 78417789 | + | N_Shore | |
| cg07516196 | 30044002 | − | S_Shore | RNF39(tss1500) |
| cg18349298 | 1.58E+08 | + | Island | RARRES1(tss1500) |
| cg16212298 | 51051954 | − | Island | LRRC48(body) |
| cg04208124 | 62272972 | − | | AHNAK(body) |
| cg26313888 | 72440429 | − | Island | LOC100101148(body); LOC541473 |
| | | | | (body) |
| cg19878482 | 1.45E+08 | − | S_Shore | MROH6(tss200) |
| cg20322093 | 72931788 | + | Island | OTOP3(tss200) |
| cg09173348 | 37322431 | + | S_Shore | ARL5C(tss200) |
| cg11182874 | 1.02E+08 | + | | |
| cg14848685 | 1.39E+08 | + | Island | KIAA1244(body) |
| cg00496961 | 1.03E+08 | + | | |
| cg07944445 | 1.55E+08 | + | | |
| cg19236794 | 1181769 | − | Island | FAM132A(body) |
| cg01511806 | 85157939 | + | Island | |
| cg25762706 | 27115867 | − | | STMN4(body) |
| cg07875335 | 48844728 | + | N_Shore | GTF2A1L(tss1500); STON1- |
| | | | | GTF2A1L(body) |
| cg13551783 | 15773099 | − | | CTRC(body) |
| cg04231541 | 2849110 | − | Island | PRSS41(body) |
| cg25136631 | 32729105 | + | N_Shore | HLA-DQB2(body) |
| cg13762474 | 42371808 | − | Island | PLA2G4D(body) |
| cg26373716 | 53474030 | + | N_Shelf | FAM150A(body) |
| cg08650934 | 45246252 | − | Island | |
| cg21894287 | 72837531 | + | Island | |
| cg20704654 | 30072118 | + | Island | LINC00028(tss1500); REM1(body) |
| cg04897644 | 1.55E+08 | + | Island | PKLR(body) |
| cg08977611 | 42953248 | + | N_Shelf | |
| cg02063817 | 27253521 | − | Island | |
| cg03300078 | 1769522 | + | Island | IFITM10(body); MOB2(body) |
| cg10841756 | 1.61E+08 | + | | PVRL4(body) |
| cg08418111 | 18433745 | − | | LDHC(tss200) |
| cg04768479 | 30095292 | + | Island | |
| cg24771017 | 48647138 | − | N_Shelf | CACNA1G(body) |
| cg13300939 | 2.02E+08 | − | | GPR37L1(tss200) |
| cg23182880 | 64374653 | − | N_Shore | NRXN2(body) |
| cg06777215 | 1.34E+08 | − | Island | GLB1L2(body) |
| cg04059863 | 1.22E+08 | − | | ILDR1(tss1500) |
| cg01081083 | 25202766 | − | S_Shore | SGSM1(body) |
| cg09737078 | 4366839 | − | Island | |
| cg19948393 | 52281810 | − | | ANKRD33(body) |
| cg20616108 | 26547234 | + | | |
| cg17546623 | 94165278 | − | | |
| cg13048466 | 22591361 | + | | |
| cg24608087 | 1.18E+08 | − | N_Shore | |
| cg16138602 | 31078790 | + | N_Shore | TSPAN11(tss1500) |
| cg23925513 | 6923526 | + | N_Shore | MIR497HG(tss1500) |
| cg20410290 | 75786172 | − | N_Shelf | |
| cg04562491 | 1.13E+08 | + | Island | DTX1(tss1500) |
| cg05072008 | 50518647 | + | Island | FIGNL1(tss1500) |
| cg14930320 | 1.71E+08 | + | S_Shelf | |
| cg08667128 | 29886885 | − | Island | MIR193A(tss200) |
| cg21901395 | 46604235 | − | N_Shore | |
| cg02451774 | 1.48E+08 | + | | NBPF8(body) |
| cg17016890 | 45556172 | − | Island | ZBTB7C(body) |
| cg00082939 | 77165293 | + | Island | ZNS503-AS2(body) |
| cg13660719 | 30852963 | + | S_Shore | DDR1(body) |
| cg18512313 | 70700886 | − | Island | |
| cg25383593 | 1.35E+08 | − | S_Shore | |
| cg13703912 | 214268 | + | N_Shore | C9orf66(body); DOCK8(tss1500) |
| cg05422049 | 1595734 | − | Island | |
| cg27277859 | 51165632 | − | Island | SHANK1(body) |
| cg10134527 | 33283015 | + | Island | TAPBP(tss1500); ZBTB22(body) |
| cg21153102 | 41252147 | − | | |
| cg23272544 | 45705429 | + | Island | AIRE(tss1500) |
| cg11438310 | 78985434 | + | | CMYA5(tss1500) |
| cg19187616 | 53012573 | − | | |
| cg12424468 | 64863373 | + | N_Shore | VPSS1(tss1500) |
| cg23998478 | 1.21E+08 | + | | |
| cg11262216 | 41723227 | − | Island | MEOX1(body) |
| cg19608003 | 57328883 | − | N_Shelf | |
| cg14769991 | 1.13E+08 | − | Island | |
| cg17700835 | 27390890 | + | | |
| cg21086940 | 53012521 | + | | |
| cg21962013 | 26586353 | + | Island | ATP8A2(body) |
| cg23769143 | 12200667 | + | S_Shore | SYN2(body); TIMP4(body) |
| cg23133355 | 2519789 | − | | |
| cg13613044 | 1.37E+08 | − | | |
| cg20436037 | 33879989 | + | Island | FAM83C(body) |
| cg26730347 | 32438947 | − | N_Shore | SLC5A1(tss200) |
| cg09163005 | 14926986 | − | N_Shore | H2AFJ(tss1500) |
| cg02299328 | 38346960 | + | N_Shore | RAPGEFL1(body) |
| cg01775897 | 73146637 | + | | DPF3(body) |
| cg21626848 | 39969267 | + | S_Shore | FKBP10(body); LEPREL4(tss1500) |
| cg05375686 | 1.31E+08 | − | Island | |
| cg07895437 | 33282971 | − | Island | TAPBP(tss1500); ZBTB22(body) |
| cg13320683 | 62761819 | − | S_Shore | RHOBTB1(tss1500) |
| cg23198707 | 1.12E+08 | + | | |
| cg11022060 | 83298969 | + | | CPEB1(body) |
| cg06636220 | 1.5E+08 | + | | |
| cg25323057 | B8730481 | + | N_Shore | ADIRF(body); AGAP11(tss200) |
| cg18063278 | 12328740 | + | N_Shore | PPARG(tss1500) |
| cg14976741 | 4872921 | + | | GALNT8(body) |
| cg20400592 | 51418041 | − | Island | DLEU7(tss200); DLEU7-AS1(body) |
| cg05339727 | 1.66E+08 | + | | SLC38A11(tss200) |
| cg24937995 | 34448038 | − | S_Shelf | |
| cg04937609 | 23852064 | + | Island | KLHL29(body) |
| cg23411869 | 74846412 | − | | |
| cg00137234 | 1.56E+08 | − | Island | RBM46(body) |
| cg18040225 | 97802849 | − | N_Shore | CCNJ(tss1500); ENTPD1-AS1(body) |
| cg00147850 | 1.45E+08 | + | | LOC100288142(body); NBPF9(body); |
| | | | | PDE4DIP(body) |
| cg02710534 | 1.41E+08 | + | N_Shelf | |
| cg10994149 | 43037123 | + | N_Shore | |
| cg239S8524 | 9289787 | − | | SRGAP3(body) |
| cg27217474 | 46697543 | − | Island | |
| cg24159514 | 11785248 | + | Island | ZNFB33P(body) |
| cg26681628 | 54210550 | + | | |
| cg10312186 | 1.7E+08 | + | S_Shore | |
| cg07208667 | 41425365 | − | S_Shore | |
| cg00594129 | 1.59E+08 | + | Island | EBF1(body) |
| cg23059946 | 2.18E+08 | − | S_Shore | IGFBP2(body) |
| cg27264390 | 53327058 | − | | |
| cg01345055 | 781041 | + | S_Shore | CPLX1(body) |
| cg14579240 | 1.4E+08 | + | | EXD3(body) |
| cg26403223 | 2.09E+08 | + | | |
| cg00489772 | 3775078 | − | Island | CEP104(tss1500); DFFB(body) |
| cg16968985 | 27313254 | − | Island | SEZ6(body) |
| cg03776087 | 2.35E+08 | + | S_Shore | TRPM8(body) |
| cg05673731 | 29565306 | + | Island | PTPRU(body) |
| cg23873996 | 1.7E+08 | − | | |
| cg12889810 | 98287347 | − | | |
| cg03240511 | 87979202 | − | | |
| cg15444185 | 67454707 | + | Island | |
| cg10099601 | 28998577 | − | S_Shore | TRI1(tss1500) |
| cg21645554 | 1.11E+08 | − | Island | COLCA1(body); COLCA2(body); COLCA2 |
| | | | | (tss1500); COLCA2(tss200) |
| cg09124496 | 41735851 | + | | INHBA(body); INHBA-AS1(body) |
| cg06399596 | 1595676 | − | N_Shelf | |
| cg14887142 | 22191761 | + | | |
| cg11427534 | 1.32E+08 | − | N_Shore | |
| cg04763519 | 1619094 | − | Island | |
| cg27329371 | 19651940 | − | S_Shelf | ALDH3A1(tss200) |
| cg24406898 | 94023308 | + | | COL1A2(tss1500) |
| cg17248924 | 1.1E+08 | − | | TMEM232(body) |
| cg11245681 | 1.76E+08 | + | N_Shelf | |
| cg16207110 | 37024401 | − | N_Shore | |
| cg01601949 | 16154325 | − | Island | |
| cg14253670 | 24802149 | − | S_Shelf | |
| cg03390419 | 76803856 | + | Island | DUPD1(body) |
| cg01497892 | 1.71E+08 | + | | |
| cg11137156 | 1.95E+08 | + | | |
| cg01827781 | 29875145 | − | S_Shore | CDIPT(tss1500); CDIPT- |
| | | | | AS1(body); CDIPT-AS1(tss200) |
| cg24790297 | 51226357 | − | N_Shore | CLEC11A(tss1500) |
| cg03862380 | 43029895 | − | | HNF4A(body); HNF4A(tss200) |
| cg20256260 | 43936981 | − | S_Shore | MATN4(body); MATN4(tss200); RBPJL |
| | | | | (body) |
| cg16681268 | 1.17E+08 | − | Island | ASZ1(tss200) |
| cg22160472 | 42944494 | + | Island | |
| cg08792388 | 1.68E+08 | − | | |
| cg09629631 | 1974724 | − | Island | CACNA2D4(body) |
| cg05550995 | 1338434 | + | | |
| cg06241901 | 43244304 | + | Island | TTBK1(body) |
| cg15234492 | 1.22E+08 | − | S_Shore | KDM2B(tss1500); KDM2B(tss200) |
| cg13526264 | 1.14E+08 | − | | |
| cg19508900 | 6639738 | − | Island | TAS1R1(body); ZBTB48(tss1500) |
| cg02026498 | 47139338 | − | Island | GNG8(tss1500) |
| cg09497409 | 8275083 | − | S_Shore | CER54(body) |
| cg10331119 | 7209566 | − | | CAMTA1(body) |
| cg01271455 | 1.57E+08 | − | S_Shore | PTPRN2(body) |
| cg01688776 | 43135973 | − | | LINC00112(tss1500); LINC00479(tss200) |
| cg06975979 | 1.5E+08 | − | Island | ACTR3C(body); LRRC61(tss1500) |
| cg06687640 | 1.6E+08 | − | | |
| cg12054981 | 42037387 | + | | PLAT(body) |
| cg12491114 | 39685524 | + | S_Shore | KRT19(tss1500) |
| cg14462779 | 76803669 | − | Island | OUPD1(body) |
| cg15730491 | 2.21E+08 | − | Island | |
| cg12302402 | 18230629 | − | Island | LOC494141(tss1500); LOC494141(tss200) |
| cg04512892 | 4785298 | + | Island | |
| cg00777271 | 1.5E+08 | − | | MTMR11(body); MTMR11(tss1500) |
| cg10833838 | 61042718 | + | N_Shore | MIR548W(body) |
| cg14731570 | 2.32E+08 | + | N_Shore | |
| cg03127898 | 62455098 | + | Island | LRRN4CL(body) |
| cg05800368 | 1.25E+08 | − | Island | TTLL11(body) |
| cg24032393 | 1.51E+08 | − | S_Shore | NOS3(body) |
| cg25026992 | 1.41E+08 | + | S_Shore | PCDHB7(body) |
| cg11785154 | 2.03E+08 | + | S_Shore | SYT2(tss1500) |
| cg13523576 | 21909636 | + | S_Shelf | DMTN(tss1500) |
| cg00056066 | 25010738 | + | N_Shore | ARHGAP21(body) |
| cg17092956 | 1.27E+08 | − | | |
| cg19846609 | 1.25E+08 | + | | |
| cg17485681 | 73565625 | − | Island | CDH23(body) |
| cg26819718 | 77872170 | + | Island | C10orf11(body) |
| cg13794863 | 36157016 | − | | |
| cg06137072 | 1.41E+08 | + | | ZBTB38(body) |
| cg15236483 | 46285682 | + | Island | |
| cg19252931 | 33721847 | − | Island | C11orf91(body) |
| cg04451175 | 16860088 | − | S_Shore | FGF20(tss1500) |
| cg11234688 | 1609660 | + | | |
| cg00341060 | 8377787 | + | Island | |
| cg15249793 | 56744413 | − | | TEX14(body) |
| cg18454045 | 88391658 | + | N_Shore | |
| cg13812393 | 742127 | + | | |
| cg20243176 | 1.13E+08 | + | N_Shore | |
| cg17191109 | 1.27E+08 | + | Island | |
| cg2180B635 | 55225133 | − | | EGFR(body) |
| cg10599948 | 49244571 | − | S_Shore | IZUMO1(body); RASIP1(tss1500) |
| cg01079738 | 1.83E+08 | − | Island | SHCB1(body) |
| cg12902206 | 52351487 | + | | DNAH1(body) |
| cg00330953 | 31733810 | + | | WVA7(body) |
| cg09614876 | 52861831 | − | | LRRC66(body) |
| cg21918786 | 1.1E+08 | + | Island | |
| cg10173186 | 12200427 | + | Island | SYN2(body); TIMP4(body) |
| cg14830045 | 1173264 | + | N_Shore | |
| cg13871843 | 9887984 | + | S_Shelf | |
| cg14629055 | 10562978 | − | Island | ANKRD33B(tss1500) |
| cg17658717 | 45077254 | + | Island | CLEC3B(body) |
| cg15484406 | 94461913 | − | | |
| Cg22803560 | 18321009 | + | | |
| cg17127823 | 71520941 | − | S_Shore | LOC286190(body); TRAM1(tss1500) |
| cg09971646 | 1.01E+08 | − | S_Shore | DLK1(body) |
| cg21697794 | 30095269 | + | Island | |
| cg00366917 | 74845154 | − | Island | MBP(tss1500) |
| cg10203922 | 1.46E+08 | − | N_Shore | HHIP(tss1500); HHIP-AS1(body) |
| cg23690302 | 1.9E+08 | + | | |
| cg20371266 | 2.33E+08 | + | S_Shore | CHRND(tss200) |
| cg05082738 | 60414918 | + | Island | LINC00301(body) |
| cg22981993 | 2.02E+08 | + | S_Shore | ALS2CR11(tss200) |
| cg09623056 | 97174912 | + | S_Shore | NEURL3(tss1500) |
| cg06847624 | 1.77E+08 | − | Island | PFN3(tss200) |
| cg27050936 | 887821 | − | Island | |
| cg01969701 | 69286145 | + | Island | |
| cg10237252 | 52977213 | − | N_Shore | TOM1L1(tss1500) |
| cg18232772 | 50165512 | + | Island | |
| cg20110349 | 28183414 | − | N_Shelf | TOB2P1(body) |
| cg26493188 | 2975209 | + | | FLYWCH1(body) |
| cg03387723 | 41708464 | + | S_Shore | SCMH1(tss1500) |
| cg10199168 | 68014338 | − | Island | DPEP3(body) |
| cg12050175 | 44754714 | + | | |
| cg15332871 | 1.68E+08 | + | | |
| cg25315503 | 45247038 | − | S_Shore | |
| cg10702227 | 49448747 | − | | |
| cg21217093 | 81871809 | + | | |
| cg20370281 | 41185803 | + | N_5hore | VPS18(tss1500) |
| cg08901520 | 2.42E+08 | − | Island | |
| cg27149931 | 1319206 | + | S_Shelf | |
| cg00425710 | 56672671 | + | S_Shore | MT1A(body) |
| cg02369542 | 41119940 | + | Island | RIMS3(body) |
| cg09391284 | 1.82E+08 | + | S_Shore | |
| cg05644480 | 45418020 | − | | APOC1(body) |
| cg17936051 | 71783345 | − | | LINC00469(body); LOC400620(body) |
| cg21855021 | 1.31E+08 | + | S_Shore | AKAP7(body) |
| cg24389359 | 1.11E+08 | − | | CCDC63(tss1500) |
| cg13205937 | 61449737 | + | S_Shore | DAGLA(body) |
| cg01431340 | 1.11E+08 | − | S_Shore | METTL24(tss1500) |
| cg17716663 | 45978434 | − | S_Shelf | FOSB(body) |
| cg03553576 | 191296 | + | N_Shore | LRRC14B(tss1500) |
| cg07142010 | 21901166 | + | | ALPL(body) |
| cg20075683 | 1.32E+08 | + | Island | CCNI2(body) |
| cg05131382 | 1.39E+08 | − | N_Shelf | |
| cg23686113 | 74660304 | − | S_Shore | STAU2(tss1500) |
| cg08121925 | 1.81E+08 | + | | |
| cg25920019 | 1.8E+08 | − | S_Shelf | |
| cg22880770 | 97166341 | + | Island | NEURL3(body) |
| cg21809624 | 63569403 | + | | |
| cg16405308 | 93723250 | − | | |
| cg17329202 | 1.14E+08 | − | S_Shore | |
| cg13871826 | 86045347 | + | N_Shore | CYR61(tss1500); DDAH1(tss1500) |
| cg19414980 | 92673176 | − | S_Shore | |
| cg00760646 | 4643865 | − | S_Shore | CXCL16(tss1500); ZMYND15(body) |
| cg27056132 | 2.28E+08 | − | N_Shore | |
| cg16060189 | 94350577 | + | N_Shore | |
| cg22463915 | 62131260 | + | S_Shelf | EEF1A2(tss1500) |
| cg21412355 | 72837386 | + | N_Shore | |
| cg19562969 | 35233582 | − | N_Shore | C20orf24(tss1500); TGIF2- |
| | | | | C20orf24(body) |
| cg02382400 | 3218487 | − | N_Shore | SLC4A11(body); SLC4A11(tss200) |
| cg07690882 | 1.29E+08 | − | Island | |
| cg14276557 | 1.4E+08 | + | | |
| cg06698332 | 1.52E+08 | − | S_Shore | S100A10(tss1500) |
| cg20664247 | 2345475 | − | N_Shore | PEX10(tss1500) |
| cg13213799 | 1.05E+08 | + | | |
| cg17241776 | 51417469 | + | Island | DLEU7(body); DLEU7-AS1(body) |
| cg01540595 | 75590960 | + | Island | |
| cg09957942 | 20968936 | + | Island | |
| cg20119532 | 749581 | + | | SLC52A3(tss1500) |
| cg13978777 | 2491825 | + | N_Shelf | |
| cg17986341 | 3649771 | + | S_Shore | TP73(body) |
| cg08286854 | 39211041 | − | | KRTAP2-2(body) |
| cg00531789 | 36927248 | + | Island | PI16(body) |
| cg02464921 | 11386866 | + | Island | |
| cg19498228 | 36342725 | + | | NPHS1(body) |
| cg17674604 | 22084959 | + | | PHYHIP(body) |
| cg15571882 | 95285252 | − | N_Shore | LINC01057(body); SLC44A3(tss1500) |
| cg13388466 | 14201744 | + | Island | |
| cg17076485 | 1.17E+08 | − | S_Shelf | |
| cg21386647 | 9289893 | + | | SRGAP3(body) |
| cg03954086 | 1.18E+08 | + | S_Shelf | |
| cg10663316 | 1.25E+08 | + | | ZNF664-FAM101A(body) |
| cg07691146 | 51797646 | − | S_Shore | FAM124A(body) |
| cg00499707 | 34679665 | − | N_Shore | EPB41L1(body); EPB41L1(tss1500) |
| cg22258315 | 48675723 | − | N_Shore | |
| cg15436123 | 3390332 | + | | RGS12(body) |
| cg03980370 | 1.39E+08 | + | S_Shore | ECT2L(body) |
| cg25629041 | 88817360 | − | | |
| cg17696006 | 26521372 | − | | |
| cg15541583 | 1.12E+08 | − | | SLC9C1(body) |
| cg01359962 | 43148002 | − | S_Shore | POMGNT2(tss1500) |
| cg00697095 | 37897076 | − | | GRB7(body) |
| cg08133641 | 55888082 | − | N_Shore | TMEM190(tss200) |
| cg05308504 | 30573013 | − | Island | |
| cg01745789 | 1.28E−08 | + | Island | |
| cg04707519 | 21799314 | + | Island | |
| cg15033653 | 1.14E+08 | + | N_Shelf | CCDC42B(tss200) |
| cg07670516 | 5019840 | − | Island | ZNF232(body) |
| cg22576165 | 29214191 | + | Island | |
| cg26185500 | 38371413 | + | | C8orf86(body) |
| cg20972453 | 20805460 | − | N_Shore | GJB6(body); GJB6(tss200) |
| cg14230238 | 6066871 | − | | RFX2(body) |
| cg07280807 | 70317239 | + | Island | |
| cg20366110 | 1.25E+08 | + | | ANXA13(body) |
| cg00288957 | 65203541 | − | N_Shore | ANKDD1A(tss1500) |
| cg12827405 | 10431475 | + | | LINC00518(body) |
| cg25145360 | 19651881 | − | S_Shelf | ALDH3A1(tss200) |
| cg11071682 | 2.1E+08 | + | S_Shelf | SERTAD4(body) |
| cg13501538 | 1616430 | + | S_Shore | |
| cg25054390 | 91720372 | + | S_Shore | GPR68(tss200) |
| cg23229568 | 2.35E+08 | + | Island | LOC100286922(body); UGT1A10(body); |
| | | | | UGT1A3(body); UGT1A4(body); |
| | | | | UGT1A5(body); UGT1A6(body); UGT1A7 |
| | | | | (body); UGT1A8(body); UGT1A9 |
| | | | | (body) |
| cg01872872 | 60382923 | − | | LINC00301(tss1500) |
| cg26112457 | 21267334 | − | S_Shore | APOB(tss1500) |
| cg08564522 | 42440079 | + | Island | |
| cg12921411 | 1.5E+08 | − | Island | SYNPO(body) |
| cg25983544 | 23713626 | − | N_Shore | PSMA8(tss200) |
| cg19743522 | 1.13E+08 | − | S_Shore | DTX1(tss200) |
| cg21750709 | 1.09E+08 | − | | DPPA4(tss1500) |
| cg22079902 | 23507644 | + | | PRDM9(tss200) |
| cg17461600 | 57983368 | − | | DAB1(body) |
| cg07297242 | 1.67E+08 | + | | |
| cg10128806 | 78977322 | + | N_Shore | FRAS1(tss1500) |
| cg18161890 | 52995250 | − | Island | KRT72(body) |
| cg09870137 | 46320026 | + | S_Shore | WNT7B(body) |
| cg05196187 | 72677504 | + | N_Shore | |
| cg13613891 | 42706106 | + | | ZBTB47(body) |
| cg06538334 | 40064867 | − | Island | |
| cg12585943 | 32121368 | + | N_Shore | LOC100507547(body); PPT2(body); |
| | | | | PPT2(tss1500); PPT2- |
| | | | | EGFL8(tss1500) |
| cg07912161 | 36929838 | − | Island | PI16(body) |
| cg09415955 | 10709885 | + | S_Shore | CASZ1(body) |
| cg06349050 | 9380756 | − | | SPSB1(body) |
| cg10195901 | 30476278 | − | Island | HORMAD2(tss200) |
| cg05394244 | 64281439 | + | N_Shore | PTP4A1(tss1500) |
| cg19825600 | 3704501 | − | | ALLC(tss1500) |
| cg00078861 | 1.54E+08 | − | | |
| cg07405121 | 2083168 | − | N_Shelf | SLC9A3R2(body); SLC9A3R2(tss200) |
| cg15407819 | 41062024 | − | S_Shore | SPTBN4(body) |
| cg01055696 | 1.64E+08 | + | Island | |
| cg08930881 | 99735202 | − | | CRTAC1(body) |
| cg09473826 | 1.76E+08 | − | N_Shore | TNR(body) |
| cg14706475 | 25218752 | + | | |
| cg01298758 | 11862611 | + | N_Shore | USP47(tss1500) |
| cg15148078 | 3480561 | − | N_Shore | C19orf77(tss200) |
| cg10949576 | 61248556 | − | | PPP1R32(tss200) |
| cg25789721 | 10370264 | + | N_Shore | ATP2B2(body) |
| cg23081026 | 29452103 | − | S_Shore | |
| cg17811845 | 89358043 | − | S_Shore | GTF2B(tss1500) |
| cg03252786 | 1.25E+08 | − | | PKNOX2(body) |
| cg00937742 | 1.33E+08 | + | S_Shore | LYPD1(tss1500) |
| cg20237610 | 1.29E+08 | + | | LAMA2(tss1500) |
| cg25844605 | 5485475 | + | | LINC00654(tss1500) |
| cg19499085 | 1796437 | − | Island | ATP8B3(body) |
| cg16643603 | 54954941 | − | | PDE18(body); PDE1B(tss1500) |
| cg11194708 | 1.64E+08 | − | Island | PACRG(body) |
| cg20249210 | 1.15E+08 | − | N_Shore | |
| cg04820679 | 33132163 | − | S_Shelf | COL11A2(body) |
| cg00381541 | 40192279 | − | Island | |
| cg15554005 | 1.29E+08 | − | | |
| cg09970023 | 61191253 | + | S_Shore | SIX4(tss1500) |
| cg24688803 | 1.05E+08 | + | S_Shore | ALDH1L2(tss1500) |
| cg14830003 | 38022657 | − | Island | DNALI1(body) |
| cg14573817 | 1.07E+08 | − | N_Shore | C12orf23(tss1500) |
| cg20703671 | 1396001 | − | S_Shore | MYO1C(body); MYO1C(tss1500) |
| cg03665132 | 2137325 | + | Island | C1orf86(body) |
| cg25341032 | 1.12E+08 | + | N_Shore | ADD3(body); ADD3-AS1(body) |
| cg18048071 | 1.59E+08 | − | | VIPR2(body) |
| cg16533838 | 46256065 | + | N_Shelf | |
| cg23079808 | 6493003 | + | Island | LTBR(body); LTBR(tss200) |
| cg18628483 | 57943326 | + | N_Shore | KIF5A(tss1500) |
| cg03995300 | 5019989 | + | S_Shore | ZNF232(body) |
| cg04769341 | 1.03E+08 | + | | TCEAL7(body) |
| cg03812309 | 46938281 | + | N_Shore | |
| cg00537210 | 23786588 | + | S_Shore | KLHL29(body) |
| cg12308391 | 1.13E+08 | + | | NCAM1(body); NCAM1-AS1(body) |
| cg18221580 | 48575897 | + | S_Shore | SUCLA2(tss1500) |
| cg13551510 | 1.32E+08 | − | Island | NTM(body) |
| cg08248579 | 17988937 | + | S_Shore | KIF13A(tss1500) |
| cg07229001 | 70897224 | + | | |
| cg13540341 | 49222985 | + | N_Shore | MAMSTR(tss200) |
| cg17937182 | 10579634 | + | | PDE4A(body) |
| cg23067299 | 323907 | − | S_Shore | AHRR(body) |
| cg05221720 | 70991229 | + | N_Shore | COL9A1(body) |
| cg01181681 | 2.23E+08 | + | S_Shore | TLR5(tss1500) |
| cg00461236 | 86704016 | + | | |
| cg18810310 | 1.04E+08 | − | | |
| cg11421972 | 16343756 | − | | HSPB7(body) |
| cg14186349 | 96964022 | + | S_Shelf | |
| cg14352032 | 28564834 | − | S_Shore | |
| cg16426764 | 1.26E+08 | − | | LHPP(body) |
| cg21171625 | 38022607 | + | Island | DNALI1(body) |
| cg16302441 | 25391830 | − | Island | POMC(tss1500) |
| cg27384918 | 87590624 | + | | |
| cg11581248 | 9887811 | − | S_Shelf | |
| cg27414481 | 6342510 | + | S_Shore | PRKCDBP(tss1500) |
| cg08787968 | 32451777 | − | N_Shore | WT1(body) |
| cg07969646 | 1.81E+08 | + | N_Shore | IER5(tss200) |
| cg10207181 | 1.45E+08 | − | | NBPF8(body) |
| cg04558175 | 50031560 | − | S_Shore | RCN3(body) |
| cg01667892 | 23507656 | + | | PRDM9(tss200) |
| cg21561315 | 52750200 | − | | |
| cg18105725 | 1.61E+08 | − | N_Shore | SLC22A1(body) |
| cg12322672 | 1.27E+08 | − | | KIRREL3(body) |
| cg04035597 | 19794539 | + | N_Shelf | |
| cg11245990 | 68621969 | + | N_Shore | |
| cg19567193 | 65066231 | − | N_Shore | RBPMS2(body) |
| cg21747160 | 49504981 | − | S_Shore | LMBR1L(tss1500) |
| cg00730441 | 59483863 | − | N_Shore | TBX2(body) |
| cg21700511 | 3713032 | − | N_Shore | HSPA12B(tss1500) |
| cg01451808 | 49676562 | + | | |
| cg18651192 | 1.26E+08 | + | Island | |
| cg24127506 | 1.02E+08 | − | N_Shore | |
| cg20254763 | 1.75E+08 | + | | |
| cg21350413 | 26577414 | − | | |
| cg18005180 | 1.39E+08 | + | N_Shore | |
| cg13073224 | 1.17E+08 | − | | |
| cg01344644 | 1.13E+08 | − | S_Shore | |
| cg07882059 | 68924751 | + | Island | LOC338694(body) |
| cg03726858 | 75243679 | − | S_Shore | |
| cg15704304 | 68881427 | − | | |
| cg06925882 | 85157885 | − | Island | |
| cg04306069 | 98175255 | − | | |
| cg03870261 | 2428514 | − | Island | LMNB2(body); TIMM13(tss1500) |
| cg06692785 | 1.81E+08 | + | S_Shore | KIAA1614(body) |
| cg06159896 | 95840443 | + | | |
| cg01692379 | 30044259 | − | S_Shore | RNF39(tss1500) |
| cg09943102 | 73157788 | − | | |
| cg21559386 | 43029652 | + | | HNF4A(body); HNF4A(tss1500) |
| cg05702280 | 34988294 | + | N_Shore | DNAJB5(tss1500) |
| cg26629730 | 43599560 | − | | SCUBE1(body) |
| cg11024728 | 1425807 | + | S_Shore | |
| cg03760518 | 3634903 | − | Island | TP73(body) |
| cg14006677 | 1.12E+08 | + | S_Shore | DIXDC1(body) |
| cg24195486 | 92788580 | + | N_Shore | SLC24A4(tss1500) |
| cg21464975 | 60828260 | − | | MARCH10(body); MIR548W(body) |
| cg01682285 | 81422160 | + | S_shore | TSHR(body) |
| cg16212279 | 1370329 | + | | |
| cg02269978 | 32847585 | − | Island | |
| cg07841866 | 1.51E+08 | + | S_Shore | NOS3(body) |
| cg07058988 | 80297159 | + | | |
| cg10729496 | 10149963 | − | Island | FANCD2OS(tss200) |
| cg00080125 | 59490613 | + | S_Shore | C17orf82(body) |
| cg11051752 | 1.58E+08 | + | | |
| cg03129014 | 20736156 | + | Island | GJA3(tss1500) |
| cg01055562 | 2.31E+08 | − | N_Shore | COG2(tss1500) |
| cg04839207 | 2.1E+08 | − | S_Shelf | SERTAD4(body) |
| cg19362874 | 3053558 | − | | |
| cg09361493 | 2.32E+08 | − | S_Shelf | |
| cg18990042 | 48399169 | − | Island | COL2A1(tss1500) |
| cg22618405 | 86038802 | − | S_Shore | |
| cg08289525 | 92939006 | + | S_Shore | ST8SIA2(body) |
| cg00474218 | 67143904 | − | | |
| cg03353445 | 13043502 | − | N_Shore | GPRC5A(tss1500) |
| cg18246298 | 1.11E+08 | − | | C11orf53(body) |
| cg02914827 | 1.27E+08 | − | Island | MMP21(body) |
| cg04122918 | 73146313 | + | | DPF3(body) |
| cg21875655 | 10660286 | − | | |
| cg26047066 | 2.21E+08 | − | Island | |
| cg04052235 | 1.23E+08 | − | | |
| cg07291889 | 11471712 | − | S_Shore | |
| cg03290213 | 78152232 | + | N_Shelf | CARD14(body); CARD14(tss200) |
| cg01422467 | 1.04E+08 | − | S_Shore | SLC9B2(tss200) |
| cg23234622 | 11621543 | − | N_Shore | |
| cg06159404 | 43846376 | + | Island | |
| cg08284496 | 37894027 | − | | GRB7(tss1500); GRB7(tss200) |
| cg08933517 | 1.04E+08 | − | | TDRD9(body) |
| cg18936052 | 28051702 | + | N_Shore | FAM76A(tss1500) |
| cg26143452 | 55907385 | − | Island | TBPL2(tss200) |
| cg19214331 | 35169260 | + | Island | MYL9(tss1500) |
| cg03475293 | 7051303 | − | N_Shore | |
| cg11941920 | 79339195 | + | | |
| cg01267165 | 11681599 | + | S_Shore | LITAF(tss1500) |
| cg15393702 | 21243529 | + | S_Shore | ANKRD29(tss1500) |
| cg22108980 | 45927824 | − | N_Shore | IGFBP1(tss200) |
| cg07038187 | 89922989 | − | S_Shore | LINC00925(body) |
| cg02873391 | 1.3E+08 | + | | |
| cg04995311 | 57630446 | + | Island | USP29(tss1500) |
| cg15249221 | 55516436 | − | N_Shore | |
| cg20852302 | 44784898 | + | S_Shelf | |
| cg11814882 | 74525125 | − | | CYGB(body); PRCD(body) |
| cg09981929 | 82296072 | + | Island | |
| cg17247293 | 1.68E+08 | + | Island | C6orf123(tss1500) |
| cg19460817 | 47575278 | − | S_Shore | CELF1(tss1500) |
| cg23579062 | 34457500 | − | Island | DNAI1(tss1500); FAM219A(body) |
| cg19797049 | 1.03E+08 | − | S_Shore | DNAJC2(tss200) |
| cg00990380 | 28601477 | − | N_Shore | |
| cg09514174 | 36916681 | − | S_Shore | OSCP1(tss1500) |
| cg18143792 | 26250290 | + | S_Shore | |
| cg08252303 | 79399007 | + | S_Shore | KCNMA1(tss1500) |
| cg17184245 | 21996585 | + | S_Shore | RAP1GAP(tss1500) |
| cg11651446 | 24446746 | + | | IL22RA1(body) |
| cg04467589 | 30044105 | + | S_Shore | RNF39(tss1500) |
| cg18227471 | 41505411 | − | S_Shore | NKX6-3(tss1500) |
| cg17974424 | 1145589 | + | | |
| cg11750736 | 10634140 | + | S_Shore | TMEM220(tss1500); TMEM220- |
| | | | | AS1(body) |
| cg18022883 | 54972054 | − | N_Shelf | |
| cg00035416 | 74660265 | − | S_Shore | STAU2(tss1500) |
| cg09511963 | 13230625 | − | Island | LOC494141(tss1500); LOC494141(tss200) |
| cg13305336 | 2.296+08 | + | | |
| cg26344606 | 21976510 | + | | HR(body) |
| cg18059464 | 1.35E+08 | − | S_Shore | GPR123(body) |
| cg23831927 | 20989645 | − | Island | CRYL1(body) |
| cg07850154 | 63461232 | + | N_Shore | RNF180(tss1500) |
| cg23651356 | 1.53E+08 | − | S_Shore | RGS17(tss1500) |
| cg12837463 | 35300228 | − | N_Shore | |
| cg07929768 | 2.32E+08 | + | | |
| cg13536265 | 24836539 | + | N_Shore | NFATC4(body); NFATC4(tss1500) |
| cg01927921 | 3787906 | − | | |
| cg12800962 | 43626437 | − | Island | |
| cg12594581 | 1.97E+08 | + | | MFI2(body) |
| cg02747254 | 29938183 | − | S_Shore | KCTD13(tss1500) |
| cg25309888 | 279B8724 | + | | |
| cg12085698 | 13983784 | − | N_Shore | SPON1(tss1500) |
| cg17351974 | 40835760 | + | Island | RPL37(tss1500) |
| cg04463638 | 19512712 | + | S_Shore | CLDN5(body) |
| cg00919055 | 1.5E+08 | + | Island | CDX1(body) |
| cg21853510 | 30285525 | − | | SECTM1(body) |
| cg06222397 | 86953019 | − | | |
| cg24694501 | 16154177 | − | Island | |
| cg05965387 | 51418053 | − | Island | DLEU7(tss200); DLEU7-AS1(body) |
| cg03632420 | 19117471 | + | | |
| cg16708623 | 1.54E+08 | − | | TRIM2(body); TRIM2(tss1500) |
| cg12111783 | 40027674 | − | Island | BCOR(body) |
| cg00571021 | 1.51E+08 | + | S_Shore | NOS3(body) |
| cg00435063 | 1.57E+08 | + | Island | PTPRN2(body) |
| cg00259472 | 1.6E+08 | − | | FABP6(body); FABP6(tss1500) |
| cg14473643 | 33282885 | − | Island | TAPBP(tss1500); ZBTB22(body) |
| cg09928766 | 51815373 | − | Island | IGLON5(body) |
| cg27511413 | 7082724 | + | | ASGR1(body) |
| cg03908908 | 76849564 | − | Island | MYO7A(body) |
| cg24161925 | 1.39E+08 | − | Island | ECT2L(tss1500) |
| cg17792806 | 1.03E+08 | + | | TCEAL7(tss200) |
| cg16105461 | 61485853 | + | N_Shelf | DAGLA(body) |
| cg04017102 | 1.11E+08 | − | | |
| cg03143118 | 42893093 | − | Island | PTCRA(body) |
| cg25500028 | 1.87E+08 | + | | |
| cg13360823 | 54496353 | − | Island | CACNG6(body) |
| cg13459217 | 2.05E+08 | − | | KLHDC8A(tss1500) |
| cg12656497 | 43099292 | + | | LINC00111(tss200) |
| cg05372495 | 1063624 | − | Island | ABCA7(body) |
| cg02540026 | 69532619 | + | N_Shore | |
| cg12384004 | 26586254 | − | N_Shore | ATP8A2(body) |
| cg03688324 | 22251017 | − | S_Shore | FGF9(body) |
| cg18648917 | 23713773 | − | | |
| cg08721802 | 1.85E+08 | + | N_Shore | |
| cg24160421 | 48675771 | + | N_Shore | |
| cg11606877 | 32076592 | + | | TNXB(body) |
| cg11152463 | 41350406 | + | S_Shore | |
| cg26214742 | 1.35E+08 | + | S_Shore | H2AFY(tss1500) |
| cg17156432 | 63261197 | − | | |
| cg06618097 | 46999444 | − | S_Shore | PNMAL2(tss1500); PPP5D1(body) |
| cg05871254 | 14213225 | + | S_Shore | HS3ST3B1(body) |
| cg21380024 | 61042803 | + | N_Shore | MIR548W(body) |
| cg05751839 | 7228268 | + | | SORCS2(body) |
| cg07570257 | 33132181 | − | S_Shelf | COL11A2(body) |
| cg09953287 | 1.3E+08 | − | Island | |
| cg20371401 | 31895493 | + | | C2(body) |
| cg03687084 | 1.62E+08 | − | | AHCTF1P1(body) |
| cg18685071 | 1.29E+08 | − | S_Shore | SMKR1(body) |
| cg04231050 | 1.33E+08 | − | | BFSP2(body) |
| cg02486415 | 52263387 | + | S_Shore | |
| cg25771683 | 5538415 | − | N_Shore | |
| cg13308964 | 69029659 | + | | |
| cg01077274 | 2.26E+08 | + | S_Shore | |
| cg26968378 | 28447087 | − | | |
| cg14278345 | 50451054 | + | S_Shore | IL17REL(body) |
| cg06353830 | 38687850 | − | | |
| cg26180255 | 46856996 | + | N_Shelf | CELSR1(body) |
| cg13246592 | 3600461 | + | S_Shore | P2RX5(tss1500); P2RX5- |
| | | | | TAX1BP3(tss1500) |
| cg03662049 | 1.64E+08 | + | Island | |
| cg02359712 | 77280597 | + | | |
| cg20531550 | 1.29E+08 | + | Island | KCP(tss200) |
| cg19515035 | 74669516 | + | S_Shore | RTKN(tss1500) |
| cg07594531 | 35225326 | + | N_Shore | GJB4(tss200) |
| cg09834706 | 39470743 | − | Island | |
| cg24324584 | 1.34E+08 | − | | |
| cg09971562 | 1.29E+08 | − | Island | |
| cg15681462 | 55880286 | − | N_Shore | IL11(body) |
| cg22376864 | 42497705 | + | N_Shore | ATP1A3(body); ATP1A3(tss200) |
| cg25096368 | 54150616 | + | | CISTR-ACT(body) |
| cg19473656 | 23623663 | − | | SLC7A8(body); SLC7A8(tss200) |
| cg23562538 | 1.45E+08 | − | Island | MAPK15(body) |
| cg21656205 | 43439688 | + | S_Shore | RIMS4(tss1500) |
| cg14525458 | 48896100 | + | N_Shore | KDELR1(tss1500) |
| cg22341310 | 48048583 | − | Island | ZNF541(body) |
| cg05654765 | 49170727 | + | | LAMB2(tss200) |
| cg25189241 | 1.72E+08 | + | N_Shore | GORASP2(body); GORASP2(tss200) |
| cg20248204 | 77461279 | − | S_Shore | KCTD12(tss1500) |
| cg19518672 | 1.32E+08 | + | Island | SEPT8(tss1500); SEPT8(tss200) |
| cg26063874 | 1260736 | − | N_Shelf | |
| cg17517709 | 18999145 | − | Island | |
| cg10851010 | 39694253 | + | | |
| cg11955878 | 1.66E+08 | − | | TRIM61(tss200) |
| cg03159409 | 1.25E+08 | − | N_Shore | FAM101A(body); ZNF664- |
| | | | | FAM101A(body) |
| cg13607218 | 71118823 | − | S_Shelf | |
| cg15644727 | 1.16E+08 | − | | |
| cg22179564 | 74199422 | + | | |
| cg05290737 | 1.23E408 | + | | HCAR1(tss200) |
| cg13245431 | 30476525 | − | Island | HORMAD2(body) |
| cg11197905 | 10459435 | + | S_Shore | ZNF5188(tss1500) |
| cg18626035 | 1.04E+08 | − | S_Shore | SLC9B2(tss200) |
| cg15922705 | 70991156 | + | N_Shore | COL9A1(body) |
| cg21242079 | 79101063 | − | N_Shore | ADAMTS7(body) |
| cg23028178 | 2.36E+08 | + | S_Shore | NID1(tss1500) |
| cg19097322 | 1.23E+08 | − | | FABP7(tss200) |
| cg07518714 | 32064945 | + | Island | TNXB(body) |
| cg19696491 | 78857124 | − | N_Shore | CHRNA5(tss1500) |
| cg16014085 | 30406122 | + | N_Shore | ZNF48(body); ZNF48(tss1500) |
| cg03871754 | 79320652 | + | Island | |
| cg24716416 | 1.89E+08 | + | | |
| cg13436378 | 71832543 | − | | DYSF(body) |
| cg13355424 | 1.57E+08 | + | Island | |
| cg21870551 | 1.17E+08 | + | Island | APOA5(body) |
| cg00521464 | 72872308 | + | S_Shelf | |
| cg08703522 | 44898785 | + | S_Shore | |
| cg08052120 | 52409580 | − | | ZNF649(tss1500) |
| cg12277789 | 1.25E+08 | − | | |
| cg06860721 | 51564909 | − | N_Shore | NCOA4(tss200) |
| cg21620139 | 31691535 | + | Island | C6orf25(body) |
| cg05382501 | 1.86E+08 | − | N_Shore | |
| cg02555807 | 40615100 | − | Island | |
| cg16166011 | 70996021 | + | S_Shore | ADD2(tss1500) |
| cg14338779 | 1.57E+08 | − | Island | PTPRN2(body) |
| cg11193342 | 1.17E+08 | − | | |
| cg22921760 | 22005368 | − | | SALL2(tss200) |
| cg16591659 | 78472290 | + | | |
| cg02960125 | 1.82E+08 | − | Island | ZNF648(body) |
| cg10012623 | 65441407 | + | S_Shelf | CHURCl-FNTBfbody) |
| cg04428163 | 1294021 | + | Island | FKBP1A- |
| | | | | SDCBP2(body); SDCBP2(body) |
| cg20115910 | 49934906 | − | S_Shore | KCNH3(body) |
| cg01730666 | 41924302 | + | | CD300LG(tss1500) |
| cg21100167 | 47295375 | − | S_Shore | PCBP3(body) |
| cg02400942 | 76006125 | + | | CSPG4(tss1500) |
| cg21325715 | 80297128 | − | | |
| cg06653052 | 1275167 | − | Island | C19orf24(tss1500) |
| cg21012874 | 88718393 | + | | MMRN2(tss1500); SNCG(body) |
| cg18947360 | 11112976 | − | | |
| cg08241939 | 2.05E+08 | + | | |
| cg26254345 | 2971684 | + | | |
| cg12843033 | 45706043 | + | Island | AIRE(body) |
| cg13903421 | 2.2E+08 | − | Island | WNT6(body) |
| cg26428830 | 45728183 | + | | |
| cg21581504 | 68545960 | + | N_Shore | CNRIP1(body) |
| cg06556497 | 76828521 | − | N_Shore | ATP9B(tss1500) |
| cg12696328 | 2.28E+08 | − | Island | |
| cg15572235 | 5183992 | + | Island | ZNF890P(body) |
| cg26102082 | 47590272 | + | Island | LOC100288866(body); NGFR(body) |
| cg04757350 | 71529128 | − | | |
| cg09452027 | 45825483 | − | N_Shore | SLC35C1(tss1500); SLC35C1(tss200) |
| cg04188937 | 3101614 | − | | |
| cg08425070 | 75864319 | − | Island | SRRM3(body) |
| cg16359657 | 41119931 | + | Island | RIMS3(body) |
| cg02853387 | 1.17E+08 | − | | DSCAML1(body) |
| cg14651210 | 1.83E+08 | + | S_Shore | |
| cg24724587 | 86044103 | + | S_Shore | DDAH1(tss200) |
| cg26544377 | 54955929 | − | N_Shore | |
| cg12720965 | 2.45E+08 | + | | KIF26B(body) |
| cg17806162 | 2472133 | − | Island | |
| cg03287299 | 5485245 | − | | LINC00654(tss200) |
| cg14578009 | 1.81E+08 | + | S_Shore | |
| cg23849826 | 39872186 | + | N_Shore | THBS1(tss1500) |
| cg00225006 | 83477872 | + | N_Shore | WHAMM(tss1500) |
| cg04850045 | 1.06E+08 | − | Island | |
| cg03921696 | 1.57E+08 | − | | |
| cg05662444 | 1.54E+08 | − | N_Shore | |
| cg20957370 | 1.53E+08 | + | | |
| cg25529304 | 43034529 | − | N_Shelf | |
| cg18290190 | 171257 | + | Island | RPH3AL(body) |
| cg17484472 | 49499189 | + | Island | |
| cg00516481 | 44073202 | + | Island | PDE9A(tss1500) |
| cg03237218 | 33760527 | + | | SLFN12(tss1500) |
| cg04369651 | 1821101 | + | | MYT1L(body) |
| cg19820919 | 9380800 | + | | SPSB1(body) |
| cg19135273 | 1.42E+08 | − | | MROH5(body) |
| cg16148593 | 22085417 | + | N_Shelf | PHVHIP(body) |
| cg24194775 | 35791475 | − | N_Shore | NPR2(tss1500) |
| cg08277369 | 84538885 | + | Island | TLDC1(tss1500) |
| cg08578641 | 34457440 | + | Island | DNAI1(tss1500); FAM219A(body) |
| cg00669347 | 86767757 | − | | |
| cg01571001 | 1.28E+08 | − | Island | |
| cg19808847 | 49836230 | + | Island | |
| cg19299755 | 1.17E+08 | + | N_Shore | |
| cg25965498 | 11746491 | + | N_Shore | VGLL4(body) |
| cg13506559 | 1.39E+08 | + | S_Shore | |
| cg18981477 | 6747166 | − | N_Shore | |
| cg06318402 | 2.45E+08 | + | Island | |
| cg16301924 | 53314226 | − | S_Shore | LECT1(tss1500) |
| cg18972998 | 55266296 | − | Island | TTC22(body) |
| cg19022717 | 1.57E+08 | − | S_Shore | LRRC71(body) |
| cg11755201 | 7551851 | + | | CAMTA1(body) |
| cg05038469 | 1.05E+08 | − | N_Shore | |
| cg04499083 | 1.51E+08 | + | N_Shelf | |
| cg16699148 | 59043255 | − | Island | TACSTD2(tss200) |
| cg24789467 | 1.32E+08 | + | Island | SMROOM1(body) |
| cg25336198 | 2182618 | − | | INS(tss200); INS-IGF2(tss200) |
| cg05106191 | 1.14E+08 | − | S_Shore | PAX8(tss200) |
| cg21903646 | 28447115 | − | | |
| cg02574464 | 57940653 | + | S_Shelf | CNGB1(body) |
| cg18730174 | 1.22E+08 | − | | MIR100HG(body) |
| cg25633271 | 36732171 | + | N_Shelf | SRCIN1(body) |
| cg14383174 | 77896174 | + | | |
| cg00270259 | 22018172 | + | S_Shelf | SFTPC(tss1500) |
| cg18774154 | 1.27E+08 | + | | |
| cg17108141 | 86044230 | + | S_Shore | DDAH1(tss200) |
| cg07477928 | 1.13E+08 | + | S_Shore | SVEP1(tss1500) |
| cg02668773 | 56356963 | + | Island | MPO(body) |
| cg01812146 | 94704178 | + | S_Shore | ARHGAP29(tss1500) |
| cg04001333 | 76045348 | + | S_Shore | FLVCR2(body) |
| cg08746853 | 14924953 | − | S_Shore | HIST4H4(tss1500) |
| cg00548822 | 66079194 | − | Island | |
| cg18337938 | 46932891 | + | Island | |
| cg20581874 | 21191754 | + | Island | |
| cg17624536 | 1.06E+08 | − | N_Shore | |
| cg00026362 | 43858741 | + | | LOC100132354(tss200) |
| cg03647927 | 36584240 | − | N_Shore | ARHGAP23(tss1500) |
| cg09613495 | 42145851 | + | Island | GUCA1A(body) |
| cg11953516 | 27719501 | − | S_Shore | FNDC4(tss1500); GCKR(tss1500) |
| cg25818214 | 64211155 | − | | PRICKLE2(tss200) |
| cg15679892 | 1267057 | + | S_Shore | |
| cg08133584 | 609679 | − | N_Shore | C16orf11(tss1500) |
| cg03610228 | 21799395 | − | S_Shore | |
| cg10130564 | 1.17E+08 | + | | LOC100652768(body); TAGLN(tss200) |
| cg13026730 | 71804139 | + | Island | EIF4E3(tss1500); GPR27(body) |
| cg15280358 | 1.28E+08 | − | | ADAM12(body) |
| cg09284655 | 70317228 | − | Island | |
| cg15078822 | 52341469 | − | Island | |
| cg01093878 | 1.32E+08 | − | N_Shore | AMER3(tss1500) |
| cg23139982 | 4763259 | − | | |
| cg22369879 | 1.37E+08 | + | | |
| cg17665699 | 19771612 | + | Island | ULK2(tss1500) |
| cg11410859 | 38687164 | + | N_Shelf | SCN5A(body) |
| cg01089602 | 1.14E+08 | + | N_Shore | F7(tss200) |
| cg10591174 | 1.11E+08 | − | | MYL2(tss200) |
| cg03272408 | 46103607 | − | S_Shore | COPZ2(body) |
| cg07033202 | 46853631 | + | N_Shore | |
| cg09386265 | 1313642 | + | S_Shore | |
| cg06080250 | 1042018 | + | N_Shore | |
| cg18208742 | 42992872 | − | S_Shelf | GFAP(body) |
| cg16419906 | 1.32E+08 | − | S_Shore | SHROOM1(tss1500) |
| cg13941021 | 1.11E+08 | − | S_Shore | PPP1CC(tss1500) |
| cg25507767 | 1.38E+08 | + | | ATP6V0A4(tss1500); TMEM213(body) |
| cg14652095 | 26018185 | + | N_Shelf | HIST1H1A(tss200) |
| cg23989110 | 27683139 | − | N_Shore | MAP3K6(body) |
| cg19481355 | 28830310 | − | N_Shore | LOC401242(body) |
| cg12523932 | 39448653 | − | Island | KLB(body) |
| cg27008631 | 49176342 | − | S_Shelf | NTN5(tss200); SEC1P(body) |
| cg11204974 | 71022665 | − | | |
| cg25417766 | 52191468 | + | | |
| cg20935489 | 1.05E+08 | + | Island | |
| cg00152942 | 57125755 | + | N_Shore | CPNE2(tss1500) |
| cg15799267 | 7942406 | − | | ALOX15B(body) |
| cg24864576 | 74499573 | − | Island | |
| cg23666491 | 25218680 | − | | |
| cg23771366 | 86510998 | − | N_Shore | PRSS23(tss1500) |
| cg18509185 | 1.08E+08 | + | | SOBP(body) |
| cg25464265 | 2.21E+08 | + | N_Shore | |
| cg16743289 | 1.75E+08 | + | N_Shore | CACYBP(tss1500) |
| cg12243267 | 57630536 | + | Island | USP29(tss1500) |
| cg12391643 | 85316997 | + | | |
| cg12317414 | 75407991 | − | S_Shore | SYNPO2L(body) |
| cg03632245 | 72590921 | − | N_Shelf | |
| cg25556008 | 2525384 | + | Island | GNG7(body) |
| cg13587180 | 42403248 | + | S_Shelf | SHISA3(body) |
| cg16750777 | 50583441 | + | Island | NKD1(body) |
| cg13072940 | 49967521 | + | S_Shore | MON1A(tss200) |
| cg20902757 | 67454892 | − | Island | |
| cg13945148 | 1.32E+08 | − | S_Shore | |
| cg11941060 | 1.34E+08 | + | Island | SRPRB(tss1500) |
| cg01794156 | 397077 | + | Island | PKP3(body) |
| cg14150727 | 2.02E+08 | + | N_Shore | LGR6(body); LGR6(tss1500) |
| cg23337866 | 1.71E+08 | − | S_Shore | |
| cg22481770 | 1.56E+08 | − | | |
| cg07141002 | 38201690 | + | Island | H1F0(body) |
| cg20793420 | 38861635 | + | N_Shelf | |
| cg09487101 | 1287428 | + | Island | EFNA2(body) |
| cg08535474 | 35149318 | + | | |
| cg15410675 | 1.54E+08 | + | S_Shore | TRIM2(body) |
| cg05357695 | 23623480 | − | | SLC7A8(body) |
| cg13459606 | 50861834 | − | S_Shore | GRB10(tss1500) |
| cg23044903 | 75118427 | + | | CPLX3(tss1500) |
| cg08906015 | 47157825 | + | | DACT3(body) |
| cg13868361 | 89357908 | − | S_Shore | GTF2B(tss1500) |
| cg05180887 | 1817263 | − | | MYT1L(body) |
| cg20475486 | 70317075 | + | Island | |
| cg21147203 | 1.35E+08 | − | S_Shelf | C5orf66(body) |
| cg05275595 | 43857874 | + | | CD177(body) |
| cg04268624 | 43037285 | + | Island | |
| cg09469223 | 56041342 | − | Island | SBK2(body) |
| cg00942918 | 26020421 | + | N_Shore | HIST1H3A(tss1500); HIST1H4A(tss1500) |
| cg26112661 | 53704558 | + | Island | LOC100507554(body); MAGOH(tss1500) |
| cg06061238 | 1.35E+08 | + | S_Shore | GPR123(body) |
| cg08282428 | 1.56E+08 | − | Island | RBM46(tss1500); RBM46(tss200) |
| cg24382070 | 22013694 | + | N_Shore | LGI3(body) |
| cg00141308 | 44725065 | − | N_Shore | |
| cg17884383 | 45848320 | − | N_Shore | KLC3(body) |
| cg23188684 | 67383651 | + | Island | DOC2GP(tss1500) |
| cg03841312 | 46508486 | + | | MIR4763(tss1500); MIRLET7A3(tss200); |
| | | | | MIRLET7B(tss1500); MIRLET7BHG |
| | | | | (body) |
| cg15254238 | 70321874 | − | S_Shore | TET1(body) |
| cg04737087 | 71205052 | − | N_Shore | ANKRD53(tss1500) |
| cg08756033 | 31480128 | − | N_Shore | MEDAG(tss200); TEX26-AS1(body) |
| cg03666300 | 88448381 | + | N_Shore | |
| cg16101008 | 10698719 | + | Island | CASZ1(body) |
| cg22674497 | 72916000 | − | Island | USH1G(body) |
| cg01189038 | 1.84E+08 | − | S_Shore | ECE2(body) |
| cg13581527 | 1708566 | − | Island | |
| cg27401562 | 40064922 | − | Island | |
| cg19478105 | 98946943 | − | S_Shore | ARHGAP19- |
| | | | | SLIT1(body); SLIT1(tss1500) |
| cg02343628 | 93790914 | + | | |
| cg15102365 | 39004656 | − | | GEMIN6(tss1500) |
| cg10564498 | 41724653 | − | | AXL(tss1500) |
| cg24235633 | 29875177 | − | S_Shore | CDIPT(tss1500); CDIPT-AS1(body) |
| cg08360599 | 1.41E+08 | − | | ZBTB38(body) |
| cg09214412 | 93614970 | − | N_Shore | RGMA(body) |
| cg00335715 | 90575459 | + | N_Shore | |
| cg17062938 | 1.55E+08 | − | S_Shore | |
| cg00520135 | 63333846 | − | N_Shore | TPM1(tss1500) |
| cg16246169 | 1.81E+08 | + | N_Shore | SOX2-OT(body) |
| cg19811683 | 16587128 | − | | |
| cg26060667 | 2.48E+08 | − | N_Shore | GCSAML(body); GCSAML(tss1500) |
| cg17057719 | 72704701 | − | S_Shore | |
| cg25821245 | 46608056 | + | S_Shore | LRRC2(tss200) |
| cg19158754 | 69794856 | − | | |
| cg19001789 | 1.11E+08 | − | N_Shore | |
| cg22760338 | 35092023 | − | | PRLR(body) |
| cg04753078 | 32063619 | + | Island | TNXB(body) |
| cg00507679 | 43251876 | − | Island | TTBK1(body) |
| cg12499872 | 58019893 | + | Island | TEPP(body) |
| cg19522930 | 1.34E+08 | − | | |
| cg21843594 | 30476285 | + | Island | HORMAD2(tss200) |
| cg09508938 | 13770042 | − | Island | |
| cg16058797 | 1.02E+08 | + | Island | |
| cg09175009 | 10912699 | + | Island | TVP23A(tss200) |
| cg05940452 | 1.19E+08 | + | Island | FOXR1(body) |
| cg14485744 | 1.19E+08 | − | | EXT1(body) |
| cg18517222 | 1.14E+08 | − | Island | |
| cg10585661 | 1.84E+08 | − | Island | FAM131A(body) |
| cg15939523 | 1.89E+08 | − | | |
| cg24750308 | 89225014 | − | S_Shore | NOX4(body); NOX4(tss1500) |
| cg11952839 | 41504887 | + | S_Shore | NKX6-3(tss200) |
| cg04629204 | 26348325 | + | | EXTL1(body) |
| cg02269643 | 70701025 | − | Island | |
| cg20391126 | 1.28E+08 | − | | |
| cg09755872 | 23245557 | − | Island | |
| cg17588350 | 60414689 | + | Island | LINC00301(body) |
| cg13448197 | 43846281 | + | Island | |
| cg03738669 | 3218476 | − | N_Shore | SLC4A11(body); SLC4A11(tss200) |
| cg19511338 | 43247938 | − | N_Shore | |
| cg27558485 | 1.96E+08 | − | | SLC51A(tss1500) |
| cg01807131 | 73856280 | + | | |
| cg06941635 | 1.32E+08 | − | S_Shore | SEPT8(tss1500) |
| cg27319263 | 64483118 | − | S_Shore | NRXN2(body) |
| cg17125472 | 69259831 | + | S_Shore | |
| cg02046423 | 2951689 | − | N_Shore | |
| cg25946374 | 24469791 | + | | IL22RA1(tss200) |
| cg23691894 | 1.12E+08 | + | N_Shore | ADD3(body); ADD3-AS1(body) |
| cg00021600 | 84003607 | + | S_Shore | NECAB2(body) |
| cg22957121 | 86659008 | + | | MGAT4C(body) |
| cg14023737 | 1.32E+08 | − | N_Shore | NTMT1(body) |
| cg00242839 | 6130578 | − | N_Shore | RBM17(tss1500) |
| cg19249231 | 2.36E+08 | − | N_Shore | GPR137B(tss1500) |
| cg11976052 | 51411839 | − | Island | KLK4(body) |
| cg15173741 | 41654618 | + | N_Shore | ANK1(body) |
| cg10091408 | 35183637 | + | | |
| cg04087740 | 71804142 | + | Island | EIF4E3(tss1500); GPR27(body) |
| cg02055264 | 27210913 | + | | |
| cg05318142 | 85999498 | − | | ATOH8(body) |
| cg11323506 | 80529135 | + | | CKMT2(tss200); RNU5D- |
| | | | | 1(body); RNU5E−1(body) |
| cg03078169 | 35629791 | − | N_Shelf | FXYD1(body); FXYD1(tss1500); FXYD1 |
| | | | | (tss200) |
| cg26787020 | 1.75E+08 | − | N_Shore | CACYBP(tss1500) |
| cg23136738 | 925521 | + | Island | AP2A2(tss1500) |
| cg22017733 | 1.63E+08 | + | S_Shelf | MAT2B(body) |
| cg26090062 | 27236425 | + | | |
| cg00605362 | 4793770 | + | S_Shelf | C16orf71(body) |
| cg03104309 | 51052080 | − | Island | LRRC4B(body) |
| cg20992663 | 1.15E+08 | − | S_Shore | TRIM33(tss1500) |
| cg20903160 | 1.39E+08 | + | Island | ECT2L(body) |
| cg26794871 | 44934712 | − | N_Shore | CDH22(body) |
| cg16899498 | 1.45E+08 | − | Island | MAFA(tss1500) |
| cg12350592 | 42031239 | − | S_Shore | PYY(body) |
| cg24599996 | 1.1E+08 | + | N_Shore | CELSR2(tss1500) |
| cg04757499 | 86016597 | − | | RGR(body) |
| cg25972714 | 88024569 | + | S_Shore | GRID1(body); MIR346(tss200) |
| cg10806711 | 1.13E+08 | − | Island | FAM19A3(body) |
| cg03655395 | 56943111 | + | N_Shore | |
| cg20654619 | 1.35E+08 | + | S_Shore | |
| cg25744277 | 28148431 | − | Island | OCA2(body) |
| cg26921881 | 1819914 | + | | |
| cg12226028 | 76484473 | − | Island | C15orf27(body) |
| cg05129477 | 21044945 | + | S_Shore | KIF17(tss1500) |
| cg22392793 | 99554416 | + | S_Shore | |
| cg18043888 | 66839187 | + | Island | RHOD(body) |
| cg19179099 | 1.06E+08 | − | | |
| cg08319294 | 43202122 | + | Island | |
| cg26046772 | 55412995 | + | S_Shore | |
| cg20752312 | 6372363 | + | N_Shore | ALKBH7(tss200) |
| cg22700880 | 28372843 | − | S_Shore | |
| cg25975485 | 54446537 | + | N_Shore | HOXC4(body); HOXC4(tss1500) |
| cg17621438 | 63461216 | + | N_Shore | RNF180(tss1500) |
| cg16484042 | 14403405 | − | | |
| cg24018756 | 300286 | − | S_Shore | IFITM5(tss1500) |
| cg10683615 | 30790683 | + | | INMT(tss1500); INMT- |
| | | | | FAM188B(tss1500) |
| cg00754947 | 50861967 | − | S_Shore | GRB10(tss1500) |
| cg20392764 | 2293117 | + | S_Shore | ASCL2(tss1500) |
| cg23846712 | 3607608 | + | S_Shore | CACTIN- |
| | | | | AS1(body); TBXA2R(tss1500) |
| cg13790268 | 1E+08 | − | N_Shore | HHIPL1(body) |
| cg21100328 | 46713446 | − | | DKFZp434J0226(tss200) |
| cg12404281 | 1.65E+08 | + | | RXRG(tss1500) |
| cg12279688 | 1.89E+08 | + | | |
| cg27274604 | 98924236 | − | | ARHGAP19- |
| | | | | SLIT1(body); SLIT1(body) |
| cg09938664 | 1.2E+08 | − | | |
| cg22566355 | 49943340 | + | Island | KCNH3(body) |
| cg18950952 | 1.11E+08 | + | N_Shore | MALL(body) |
| cg07472953 | 38170906 | − | | CSF3(tss1500) |
| cg09142166 | 44276013 | + | | PNPLA5(body) |
| cg12091331 | 42065314 | − | | PLAT(tss200) |
| cg02874016 | 2017649 | − | Island | CACNA2D4(body) |
| cg23526676 | 31734458 | + | | VWA7(body) |
| cg26181818 | 2.33E+08 | + | Island | ALPI(body) |
| cg21376136 | 1.53E+08 | + | | CACNB4(body); CACNB4(tss200) |
| cg03379797 | 1.39E+08 | + | | NHSL1(body) |
| cg12758090 | 80543408 | + | N_Shore | |
| cg15469871 | 27541418 | + | Island | LRRC37A6P(tss200) |
| cg19343034 | 49223253 | − | Island | MAMSTR(tss1500) |
| cg13017345 | 1.12E+08 | + | N_Shore | MXI1(tss1500) |
| cg01328833 | 4277322 | − | N_Shore | SHD(tss1500) |
| cg27395056 | 43221220 | − | N_Shore | ACBD4(body) |
| cg01411366 | 1.12E+08 | + | | SLC9C1(body) |
| cg21543918 | 1.39E+08 | + | | NHSL1(body) |
| cg01908551 | 81036175 | − | N_Shore | METRNL(tss1500) |
| cg05123976 | 45949677 | − | Island | |
| cg12299599 | 25934722 | − | | |
| cg10883038 | 1.15E+08 | + | N_Shore | DENND2C(body) |
| cg02978784 | 28460106 | + | S_Shelf | |
| cg17735531 | 611349 | − | Island | HCN2(body) |
| cg13557773 | 1.15E+08 | − | Island | RASA3(body) |
| cg00531453 | 1.24E+08 | + | S_Shore | TMEM229A(tss1500) |
| cg11476737 | 2815189 | + | Island | |
| cg23123165 | 54901015 | − | Island | |
| cg03708694 | 44489577 | + | | |
| cg11028624 | 13983793 | − | N_Shore | SPON1(tss1500) |
| cg00909806 | 2.13E+08 | + | S_Shore | |
| cg24983120 | 74499380 | + | Island | |
| cg27305303 | 26781231 | − | N_Shelf | OTOF(body) |
| cg17802633 | 68871468 | − | S_Shore | PROKR1(tss1500) |
| cg19551055 | 1.35E+08 | + | N_Shelf | |
| cg17341345 | 86709409 | − | S_Shore | CCNH(tss1500) |
| cg05210804 | 33282896 | + | Island | TAPBP(tss1500); ZBTB22(body) |
| cg09250458 | 1139916 | + | Island | C1QTNF8(body) |
| cg02787980 | 28174814 | − | | PNOC(body) |
| cg19793809 | 1.32E+08 | − | Island | SHROOM1(tss200) |
| cg06017490 | 25893245 | + | S_Shore | |
| cg02792821 | 44451783 | + | Island | |
| cg04247717 | 1.32E+08 | + | Island | ARHGEF4(body) |
| cg26250093 | 24397649 | − | Island | FAM228A(tss1500) |
| cg13692134 | 74350132 | + | Island | |
| cg27310219 | 81240445 | + | Island | |
| cg25293806 | 55889387 | + | Island | TMEM190(body) |
| cg00031973 | 47783848 | + | | |
| cg22486928 | 96251729 | − | N_Shore | SNRPF(tss1500) |
| cg05730092 | 1.01E+08 | + | S_Shore | BEGAIN(tss1500) |
| cg26051413 | 2292902 | + | Island | ASCL2(tss1500) |
| cg04595372 | 49681233 | − | | CRISP2(body) |
| cg24378227 | 1396771 | + | S_Shelf | |
| cg27052709 | 76495017 | + | N_Shelf | DNAH17(body); DNAH17- |
| | | | | AS1(body) |
| cg06768010 | 74729502 | + | Island | LBX2(body); LBX2-AS1(tss1500) |
| cg04788926 | 76617198 | + | Island | |
| cg15256387 | 14031941 | + | S_Shore | ETV1(tss1500) |
| cg13853761 | 64863232 | + | N_Shore | VPS51(tss1500) |
| cg15280185 | 1.04E+08 | + | | |
| cg14259096 | 3746221 | − | N_Shelf | |
| cg19218607 | 49925924 | + | N_Shelf | |
| cg22361859 | 37123638 | + | | FBXO47(body) |
| cg08002151 | 47968096 | + | Island | |
| cg07181395 | 2.19E+08 | − | | |
| cg02019125 | 1.56E+08 | + | | |
| cg19513207 | 1.43E+08 | + | Island | |
| cg03379552 | 99734662 | − | | CRTAC1(body) |
| cg02853553 | 44258054 | − | N_Shore | CAMK2B(body) |
| cg26459819 | 1.11E+08 | − | Island | COLCA1(body); COLCA2(body); COLCA2 |
| | | | | (tss1500); COLCA2(tss200) |
| cg14555100 | 46992053 | + | N_Shore | GPRIN2(tss1500) |
| cg13422804 | 1.4E+08 | + | S_Shore | AGPAT2(tss1500) |
| cg12210255 | 72838671 | − | Island | |
| cg08144358 | 1.57E+08 | + | | |
| cg03225580 | 38256147 | + | N_Shore | |
| cg26132737 | 1.98E+08 | − | Island | ANKRD44(body) |
| cg12019614 | 11353996 | − | Island | DDCK6(body) |
| cg11500727 | 42894425 | − | S_Shelf | CNFN(body) |
| cg12638745 | 40816464 | + | N_Shore | LCA5L(tss1500); SH3BGR(tss1500) |
| cg07963854 | 1.31E+08 | + | S_Shore | |
| cg12582654 | 1.29E+08 | + | Island | KCP(tss200) |
| cg13700073 | 79495324 | − | Island | FSCN2(tss200) |
| cg21516302 | 1.31E+08 | − | | |
| cg06377543 | 23713657 | + | N_Shore | PSMA8(tss200) |
| cg10194870 | 54598545 | − | N_Shore | OSCAR(body) |
| cg24779381 | 74466794 | − | N_Shore | ISLR(body); ISLR(tss200) |
| cg04537050 | 3149195 | + | | ZSCAN10(body) |
| cg22533683 | 29887049 | + | Island | MIR193A(body) |
| cg20003638 | 44687730 | − | S_Shore | SLC12A5(body) |
| cg08739828 | 33768144 | + | Island | MOCOS(body) |
| cg02372856 | 49340974 | − | | |
| cg17437419 | 95400067 | + | S_Shore | LOC440311(body) |
| cg13296371 | 4250746 | + | S_Shore | TMEM128(tss1500) |
| cg10601230 | 1.63E+08 | + | | PACRG(body) |
| cg03560996 | 31733908 | − | | VWA7(body) |
| cg17564844 | 23413784 | − | S_Shore | GNAZ(body); RTDR1(body) |
| cg14130039 | 32121225 | − | N_Shore | LOC100507547(body); PPT2(tss1500); |
| | | | | PPT2(tss200); PPT2- |
| | | | | EGFL8(tss1500) |
| cg00900933 | 37322028 | − | Island | ARL5C(body) |
| cg02962597 | 1.18E+08 | − | S_Shore | NOS1(tss200) |
| cg11898431 | 1.59E+08 | + | | |
| cg18162670 | 1.04E+08 | − | Island | |
| cg10001845 | 20751945 | + | Island | |
| cg06453241 | 2.18E+08 | − | | |
| cg22261895 | 14720100 | − | N_Shore | PLBD1(body) |
| cg14691388 | 1.41E+08 | − | N_Shelf | KCNK9(body) |
| cg25129541 | 53273196 | − | N_Shore | |
| cg23594693 | 41703970 | − | S_Shore | TFEB(body); TFEB(tss1500) |
| cg15192101 | 4763372 | − | | |
| cg21139046 | 14339700 | + | S_Shore | |
| cg22610676 | 1.56E+08 | − | S_Shore | C1orf61(body) |
| cg04289314 | 1.31E+08 | + | Island | |
| cg25447806 | 1.57E+08 | − | Island | LlNC00880(body) |
| cg27110277 | 16860121 | − | S_Shore | FGF20(tss1500) |
| cg08757448 | 1.05E+08 | + | N_Shore | C10orf32(tss200); C10orf32- |
| | | | | ASMT(tss200) |
| cg20495333 | 1.21E+08 | + | S_Shore | INHBB(body) |
| cg11503875 | 46285985 | + | Island | |
| cg18847009 | 70175826 | − | | |
| cg05477174 | 65648771 | + | S_Shore | IGDCC3(body) |
| cg15482422 | 66956240 | − | Island | RRAD(body) |
| cg22235293 | 31691539 | + | Island | C6orf25(body) |
| cg21125503 | 1.25E+08 | + | S_Shore | |
| cg11077026 | 1.6E+08 | + | S_Shore | IGSF9(tss1500) |
| cg26382173 | 1.06E+08 | − | Island | TMEM121(body) |
| cg14637470 | 1.32E+08 | + | N_Shore | FAM73B(tss1500) |
| cg07827796 | 33622959 | − | Island | WDR88(tss200) |
| cg06621452 | 1.11E+08 | + | N_Shore | MALL(body) |
| cg04264925 | 1.35E+08 | + | N_Shelf | |
| cg11150807 | 43354902 | + | | |
| cg12938128 | 64483691 | + | S_Shelf | NRXN2(body) |
| cg07576591 | 1.77E+08 | + | S_Shore | |
| cg08045932 | 61659980 | − | N_Shore | LOC63930(body) |
| cg13388692 | 86081680 | − | N_Shore | |
| cg22998028 | 42247910 | − | | ASB16(tss200) |
| cg06161375 | 26394593 | + | | TRIM63(tss1500) |
| cg08350509 | 3028220 | + | N_Shore | TLE2(body) |
| cg16922763 | 1.12E+08 | + | N_Shore | |
| cg08756640 | 88392351 | + | S_Shore | |
| cg06722121 | 72934409 | + | S_Shore | OTOP3(body) |
| cg01618988 | 61635995 | + | N_Shore | |
| cg09084256 | 32555286 | + | | C22orf42(tss200) |
| cg09134760 | 60921084 | + | N_Shore | |
| cg24654266 | 1.15E+08 | + | Island | DPP10(body) |
| cg11630226 | 1.44E+08 | − | Island | LY6K(body) |
| cg13717553 | 51067014 | + | N_Shelf | LRRC4B(body) |
| cg16489360 | 1.3E+08 | + | S_Shore | TMEM209(tss1500) |
| cg07148341 | 43714275 | + | | |
| cg07088882 | 8038043 | − | N_Shelf | |
| cg20473155 | 10688228 | + | | |
| cg10060499 | 39378734 | − | | |
| cg10257302 | 55907427 | + | Island | TBPL2(tss200) |
| cg24302529 | 45923083 | − | Island | ALOX5(body) |
| cg22442730 | 62179233 | + | S_Shore | SRMS(tss1500) |
| cg05826295 | 1.14E+08 | + | | |
| cg12265130 | 59437344 | − | S_Shore | PATL1(tss1500) |
| cg20121927 | 1.66E+08 | + | N_Shelf | LINC00473(body) |
| cg03630015 | 2.2E+08 | − | N_Shore | WNT10A(body) |
| cg01978213 | 41866309 | − | N_Shore | |
| cg01832802 | 1.04E+08 | − | S_Shore | LINC00605(tss1500) |
| cg22807681 | 1.75E+08 | − | | |
| cg27361011 | 1.21E+08 | − | Island | |
| cg05831517 | 86337750 | − | Island | |
| cg15062725 | 3831364 | − | Island | ZFR2(body) |
| cg17881600 | 17012979 | − | | |
| cg21647560 | 2.14E+08 | + | | |
| cg08069902 | 88675853 | + | | KCNK10(body) |
| cg17278401 | 66462662 | + | S_Shelf | MAST4(body) |
| cg20960833 | 1.55E+08 | + | S_Shore | |
| cg27269130 | 54985351 | − | S_Shelf | CDC42EP5(tss1500) |
| cg12854815 | 1.6E+08 | + | | |
| cg27241889 | 833012 | − | S_Shore | ERICH1-AS1(body) |
| cg15671996 | 30140325 | − | S_Shore | TRIM15(body) |
| cg00157359 | 66233183 | + | N_Shore | PELI3(tss1500) |
| cg01443946 | 5936980 | − | Island | ANO2(body) |
| cg20703003 | 1.16E+08 | − | N_Shore | |
| cg19956113 | 1.84E+08 | + | S_Shore | CHRD(body) |
| cg14319451 | 63685777 | − | Island | RCOR2(tss1500) |
| cg22318514 | 31650850 | − | Island | |
| cg11787508 | 26761337 | − | S_Shore | |
| cg23598212 | 1769289 | − | Island | IFITM10(body); MOB2(body) |
| cg14546466 | 11796974 | − | Island | |
| cg22535729 | 55533955 | + | Island | RP1(body) |
| cg01518016 | 39695237 | − | S_Shore | SYCN(tss1500) |
| cg22395765 | 1.57E+08 | + | S_Shore | PTPRN2(body) |
| cg08577298 | 1.8E+08 | − | Island | |
| cg27053299 | 1.01E+08 | + | Island | CLYBL(body) |
| cg27570575 | 85917536 | + | | |
| cg18324432 | 56306697 | − | | |
| cg17672101 | 3825630 | + | S_Shore | ZFR2(body) |
| cg26256793 | 1.04E+08 | − | | COL11A1(tss1500) |
| cg10210594 | 2.08E+08 | + | Island | |
| cg03432955 | 29795501 | + | N_Shore | HLA-G(body) |
| cg00593462 | 1.11E+08 | − | | |
| cg13329862 | 30850876 | − | N_Shore | DDR1(tss1500) |
| cg23178550 | 27127576 | + | Island | |
| cg25913761 | 90727560 | − | N_Shore | SEMA4B(tss1500) |
| cg25480418 | 88583785 | − | Island | |
| cg12955789 | 1.8E+08 | − | Island | RASGEF1C(body) |
| cg25458010 | 1.33E+08 | − | N_Shore | GALNT9(body) |
| cg10905012 | 64374819 | − | Island | NRXN2(body) |
| cg10414829 | 88719259 | − | | SNCG(body) |
| cg02013987 | 44529223 | + | N_Shore | ZNF222(tss1500) |
| cg09988676 | 2.33E+08 | − | S_Shore | CHRND(body) |
| cg18825119 | 1.63E+08 | − | S_Shore | PACRG(body); PARK2(tss1500) |
| cg18001869 | 39966711 | − | | CACNA1I(tss200) |
| cg25877436 | 27998191 | − | N_Shore | GTF3A(tss1500) |
| cg07774964 | 65133105 | − | N_Shore | PLEKHO2(tss1500) |
| cg19093295 | 45715517 | − | | |
| cg18735956 | 34113908 | + | Island | GRM4(body) |
| cg22380178 | 29980695 | + | | |
| cg10145449 | 1.45E+08 | − | Island | SPATC1(body) |
| cg04094751 | 3725861 | − | | HSPA12B(body) |
| cg14182974 | 4791918 | + | N_Shore | RCL1(tss1500) |
| cg16436164 | 19936663 | − | | |
| cg04768368 | 1.24E+08 | + | | TACC2(body) |
| cg09200774 | 41877387 | + | N_Shore | |
| cg14584255 | 1.63E+08 | + | S_Shore | PACRG(body); PARK2(tss1500) |
| cg23258611 | 70536160 | − | N_Shore | LINC00673(body) |
| cg21697944 | 49658797 | − | N_Shelf | HRC(tss200) |
| cg15860114 | 88133956 | + | N_Shelf | |
| cg11437810 | 69744829 | + | N_Shore | RPLP1(tss1500) |
| cg05313129 | 58192883 | + | Island | LINC00588(body) |
| cg02481697 | 8928012 | − | S_Shore | NTN1(body) |
| cg11886639 | 49704653 | − | | |
| cg05264816 | 44237023 | + | Island | LOXHD1(tss200) |
| cg25593625 | 24397689 | − | | MYOM3(body) |
| cg16225804 | 29210759 | + | | |
| cg15836046 | 2767242 | + | | |
| cg15174248 | 1.5E+08 | + | | CAMK2A(tss1500) |
| cg14437996 | 91794687 | + | S_Shore | SHC3(tss1500) |
| cg09136878 | 2.36E+08 | + | | |
| cg14227911 | 1.13E+08 | − | | |
| cg14254748 | 62660421 | − | N_Shore | L1TD1(tss200) |
| cg12564962 | 38377938 | − | Island | RIPPLY3(tss1500) |
| cg00310410 | 1.06E+08 | − | N_Shore | |
| cg12146150 | 8958090 | + | | NTN1(body) |
| cg01123965 | 39866890 | + | Island | |
| cg03176729 | 1.24E+08 | − | S_Shore | HTRA1(body) |
| cg01806972 | 66314378 | − | Island | ACTN3(body); ZDHHC24(tss1500) |
| cg23132774 | 1.15E+08 | + | Island | RASA3(body) |
| cg19871173 | 2.3E+08 | − | | |
| cg14508358 | 6716417 | + | Island | |
| cg23063807 | 506174 | − | S_Shelf | |
| cg06662140 | 18809297 | − | Island | KLHDC7A(body) |
| cg04671739 | 1.51E+08 | − | | ASB10(tss1500); ASB10(tss200) |
| cg03522256 | 1.14E+08 | + | | |
| cg06533798 | 59323070 | + | | |
| cg25498045 | 36024375 | + | Island | GAPDHS(body) |
| cg19252333 | 29583372 | + | N_Shelf | PTPRU(body) |
| cg17359265 | 1.79E+08 | − | S_Shore | ADAMTS2(body) |
| cg24439713 | 86081713 | − | Island | |
| cg22177286 | 2.32E+08 | − | N_Shore | ITM2C(tss1500) |
| cg08541962 | 35894321 | − | N_Shelf | GJD4(tss200) |
| cg02059315 | 3989432 | + | S_Shore | |
| cg20160695 | 90596588 | + | Island | |
| cg24324441 | 1.59E+08 | − | N_Shore | MFSD1(tss1500) |
| cg15463284 | 18477534 | − | Island | LDHAL6A(body) |
| cg12462883 | 1.12E+08 | + | | SLC9C1(tss200) |
| cg23711873 | 77035993 | − | | |
| cg13947929 | 43863356 | + | S_Shore | CRHR1(body) |
| cg03312205 | 6832737 | − | N_Shore | COPS7A(tss1500) |
| cg14554415 | 69222988 | + | Island | MIR548H4(body); NOX5(body); SPESP1 |
| | | | | (body) |
| cg06067425 | 1.41E+08 | − | N_Shore | LOC152586(body) |
| cg22325294 | 30043829 | + | S_Shore | RNF39(tss1500) |
| cg27511599 | 32358540 | − | | |
| cg20639805 | 1.12E+08 | − | S_Shore | RBM20(body) |
| cg06312846 | 1410167 | + | | |
| cg27666046 | 80279077 | + | Island | SECTM1(body) |
| cg02465887 | 1.55E+08 | − | N_Shore | DCHS2(body) |
| cg06061081 | 77299960 | + | N_Shore | AQP11(tss1500) |
| cg27424326 | 1.29E+08 | + | Island | |
| cg12067764 | 1.54E+08 | + | | |
| cg08052629 | 38746615 | − | N_Shore | PPP1R14A(body) |
| cg26557756 | 42278096 | + | S_Shore | GLI3(tss1500) |
| cg14036033 | 35206233 | − | | |
| cg07241660 | 69259336 | − | S_Shore | |
| cg23878875 | 48422933 | − | N_Shore | XYLT2(tss1500) |
| cg09265392 | 49910216 | − | Island | |
| cg02276974 | 78634235 | + | S_Shore | CRABP1(body) |
| cg22068464 | 1.22E+08 | + | | |
| cg14214165 | 2.19E+08 | − | | TNS1(body) |
| cg04142296 | 38757532 | − | N_Shore | PLEKHA2(tss1500) |
| cg11235602 | 39543776 | − | Island | MOBP(body) |
| cg16243197 | 1.83E+08 | + | N_Shore | DHX9(tss1500) |
| cg14026325 | 1.45E+08 | + | Island | |
| cg23810282 | 43037519 | + | Island | |
| cg09857070 | 2822901 | − | | |
| cg04197086 | 1.13E+08 | − | N_Shore | |
| cg17207000 | 72321767 | + | N_Shore | KIF19(tss1500) |
| cg21356710 | 24234017 | − | S_Shore | MFSD2B(body) |
| cg00871787 | 79920595 | − | Island | |
| cg25882597 | 39969542 | + | S_Shore | LPIN3(tss200) |
| cg25849642 | 1.13E+08 | − | Island | |
| cg22858288 | 2930322 | + | Island | SLC22A18(body) |
| cg05692746 | 1.01E+08 | + | N_Shore | LONRF2(body) |
| cg19399165 | 27301252 | + | | EMILIN1(tss200) |
| cg09807524 | 17569556 | + | S_Shelf | OTOG(body) |
| cg20733761 | 47295215 | + | Island | PCBP3(body) |
| cg19683073 | 79553267 | + | S_Shore | SERINC5(tss1500) |
| cg19257464 | 1.31E+08 | + | N_Shore | |
| cg06531916 | 1.21E+08 | + | | |
| cg04010205 | 4118274 | − | Island | GLIS3(body) |
| cg24546723 | 48048615 | − | N_Shore | DLX4(body) |
| cg02050426 | 59221458 | − | Island | CDH20(body) |
| cg07643267 | 43969769 | + | S_Shore | LYPD3(body) |
| cg10287786 | 1.18E+08 | − | N_Shore | DSCAML1(body) |
| cg05609335 | 75789393 | − | Island | |
| cg07348481 | 61355119 | − | Island | |
| cg02055607 | 1.41E+08 | − | | |
| cg24345184 | 19751899 | + | Island | TBX1(body) |
| cg03866211 | 45939109 | − | Island | ALOX5(body) |
| cg23349301 | 46697115 | − | N_Shore | |
| cg13679679 | 28282245 | + | N_Shore | CMC1(tss1500) |
| cg07674022 | 1.23E+08 | − | S_Shore | TRPC3(body); TRPC3(tss200) |
| cg16763089 | 5485284 | + | | LINC00654(tss200) |
| cg09318162 | 32847842 | + | Island | |
| cg01987516 | 1.01E+08 | − | Island | |
| cg03981535 | 2.02E+08 | + | N_Shore | RNPEP(tss1500) |
| cg24148085 | 72704603 | − | Island | |
| cg16317457 | 1.23E+08 | + | Island | |
| cg07447742 | 49793170 | + | N_Shore | MLNR(tss1500) |
| cg15383087 | 36910144 | − | S_Shore | ZFP82(tss1500) |
| cg00628788 | 33529997 | + | Island | |
| cg05497750 | 23765381 | − | N_Shore | CHP2(tss1500) |
| cg19092396 | 1143751 | + | N_Shore | |
| cg06156640 | 3704363 | − | | ALLC(tss1500) |
| cg02309230 | 4612064 | + | | |
| cg11923441 | 1.44E+08 | − | | |
| cg16348254 | 43837992 | + | Island | |
| cg07981328 | 21724633 | + | | |
| cg01084740 | 61748243 | + | S_Shore | TMEM30B(body) |
| cg10645091 | 76445055 | − | | C13orf45(tss200) |
| cg01812577 | 62689122 | − | N_Shore | CHRM1(tss200) |
| cg16346321 | 10709185 | + | S_Shore | CASZ1(body) |
| cg12673429 | 11150043 | + | S_Shore | PIEZO2(ts515O0) |
| cg05566455 | 1.29E+08 | + | | C11orf45(body); KCNJ5(body) |
| cg00123104 | 9884732 | − | S_Shore | CLSTN1(tss200) |
| cg11334406 | 4546450 | + | S_Shore | ALOX15(tss1500) |
| cg05180451 | 27185764 | + | | |
| cg23312397 | 96670738 | − | | BDKRB2(tss1500) |
| cg18922062 | 1.8E+08 | − | N_Shore | BTNL9(body) |
| cg12072164 | 44306565 | + | S_Shelf | LYPD5(body) |
| cg12155036 | 1.94E+08 | + | S_Shore | |
| cg25220063 | 2.48E+08 | + | Island | |
| cg00056257 | 6901652 | − | N_Shore | GRM7(tss1500) |
| cg17093267 | 1.12E+08 | + | S_Shore | XPNPEP1(tss1500) |
| cg06153927 | 1.71E+08 | + | N_Shelf | |
| cg10685359 | 87814065 | − | S_Shore | C4orf36(tss1500) |
| cg17620300 | 6058563 | − | Island | |
| cg21864304 | 11746722 | − | Island | VGLL4(body) |
| cg18637459 | 1.09E+08 | + | | TMEM119(tss200) |
| cg07469063 | 69755704 | + | | |
| cg23513363 | 43817997 | + | N_Shore | |
| cg17425810 | 48449748 | − | N_Shore | TRABD2B(body) |
| cg11251135 | 1853911 | − | | RTN4RL1(body) |
| cg11414913 | 2799662 | + | | |
| cg15050310 | 86617582 | + | Island | |
| cg00500770 | 47012991 | − | N_Shore | |
| cg08530537 | 68014374 | − | Island | DPEP3(body) |
| cg09382492 | 74463681 | + | | AANAT(body) |
| cg15822328 | 1072197 | − | N_Shore | LOC254O99(tss200) |
| cg04829830 | 1.05E+08 | − | | CEP170B(body) |
| cg07421287 | 1.11E+08 | − | S_Shore | KCNA3(tss1500) |
| cg24439334 | 2345374 | + | N_Shore | PEX10(tss1500) |
| cg06376715 | 3634918 | + | Island | TP73(body) |
| cg10941424 | 5294958 | − | | |
| cg03652339 | 1.12E+08 | − | Island | DUSPS(body) |
| cg17207736 | 1.42E+08 | − | Island | SLC45A4(body) |
| cg00599163 | 1.62E+08 | − | N_Shore | |
| cg02757179 | 25384809 | − | S_Shore | POMC(body) |
| cg25343227 | 42353589 | − | N_Shore | LINC00634(body) |
| cg14095959 | 77723415 | + | Island | |
| cg22234712 | 96085385 | − | Island | MIR3150B(tss200) |
| cg10081469 | 61748335 | + | S_Shore | TMEM30B(body) |
| cg04777352 | 20208197 | − | | |
| cg08889843 | 56048518 | − | S_Shore | SBK2(tss200) |
| cg20925811 | 44638517 | − | N_Shore | MMP9(body) |
| cg03364381 | 43099460 | − | − | LINC00111(tss200) |
| cg11684572 | 28147928 | − | Island | OCA2(body) |
| cg20980321 | 32144667 | + | | AGPAT1(body); AGPAT1(tss1500); RNF5(tSS1500) |
| cg11549848 | 24464707 | + | S_Shore | C1QTNF9B- |
| | | | | AS1(body); MIPEP(tss1500) |
| cg02713669 | 1.1E+08 | − | Island | SH3RF3(body); SH3RF3-AS1(tss200) |
| cg10551185 | 1.78E+08 | − | | |
| cg00397764 | 1.16E+08 | + | S_Shore | TDRD1(body) |
| cg10672681 | 1.12E+08 | − | N_Shore | ABHD10(tss200) |
| cg24003560 | 2.21E+08 | + | N_Shore | C1orf115(tss1500) |
| cg22748407 | 20947311 | − | S_Shore | USP22(tss1500) |
| cg02481789 | 40805506 | − | N_Shore | |
| cg06819763 | 85294457 | − | | |
| cg25940248 | 39448464 | + | Island | KLB(body) |
| cg25925441 | 1.54E+08 | + | S_Shore | RPRM(tss1500) |
| cg15600238 | 27260585 | + | S_Shore | |
| cg14221644 | 1.39E+08 | + | S_Shelf | |
| cg20652899 | 1.18E+08 | + | S_Shore | FXYD6(body); FXYD6(tss200); FXYD |
| | | | | 6-FXYD2(tss200) |
| cg01076229 | 49681334 | − | | CRISP2(tss200) |
| cg00174500 | 23846479 | − | | CMTMS(body) |
| cg04546557 | 27262483 | − | N_Shelf | |
| cg05601974 | 1.51E+08 | + | N_Shore | SMARCD3(body) |
| cg23575603 | 50707753 | − | Island | |
| cg23989012 | 1.48E+08 | − | S_Shore | NBPF8(body) |
| cg19274341 | 8143891 | − | | |
| cg25112202 | 72704468 | − | Island | |
| cg20806180 | 39852862 | + | Island | MGAT3(tss1500) |
| cg26230651 | 1.14E+08 | − | Island | |
| cg19208681 | 56769628 | − | N_Shore | RAD51C(tss1500); TEX14(tss1500) |
| cg07589899 | 62020677 | + | | |
| cg16596957 | 87575150 | − | | |
| cg12984635 | 44032076 | + | S_Shore | ETHE1(tss1500) |
| cg21004358 | 1.87E+08 | + | Island | F11-AS1(body) |
| cg07444554 | 1.39E+08 | − | | NRG2(body) |
| cg11566975 | 49170849 | + | | LAMB2(tss1500) |
| cg08030082 | 25391839 | + | Island | POMC(tss1500) |
| cg16817668 | 32055006 | − | S_Shore | |
| cg18180846 | 1.03E+08 | + | S_Shore | LBX1-AS1(body) |
| cg03464655 | 1.66E+08 | − | | SLC38A11(body) |
| cg05812599 | 72146256 | + | S_Shore | CLPB(tss1500) |
| cg15865892 | 1.07E+08 | + | | |
| cg09571848 | 64330935 | + | N_Shore | |
| cg03366850 | 28998595 | − | S_Shore | TRIL(tss1500) |
| cg05754122 | 37499741 | − | | TMPRSS6(tss200) |
| cg00255882 | 1.29E+08 | + | S_Shore | WDR33(tss1500) |
| cg00237876 | 41924043 | + | | CD300LG(tss1500) |
| cg24479859 | 1.13E+08 | + | | |
| cg15571353 | 24528833 | + | | LOC284632(body) |
| cg10647465 | 1.27E+08 | − | | KIRREL3(body) |
| cg09012594 | 21464258 | − | S_Shore | NEBL(tss1500) |
| cg15263021 | 61273282 | + | N_Shelf | |
| cg00761968 | 53314142 | + | S_Shore | LECT1(tss200) |
| cg04436389 | 46417734 | + | Island | NANOS2(body) |
| cg16550453 | 1.16E+08 | − | Island | TDRD1(tss200) |
| cg10184431 | 27783226 | − | S_Shore | HIST1H2AJ(tss1500); HIST1H2BM(body) |
| cg03960424 | 57042304 | + | Island | APCDD1L(body) |
| cg12945707 | 33132241 | − | S_Shelf | COL11A2(body) |
| cg13460850 | 1.84E+08 | + | S_Shore | |
| cg26717563 | 3006111 | − | N_Shore | TLE2(body) |
| cg10978034 | 33129988 | + | S_Shore | |
| cg04938738 | 1689429 | + | | MOB2(body) |
| cg19645255 | 1202252 | − | | TUSC5(body) |
| cg21219373 | 45885940 | + | Island | PPP1R13L(body) |
| cg06761424 | 1.15E+08 | − | Island | KIAA1210(tss1500) |
| cg27006947 | 63924280 | − | N_Shore | |
| cg06888406 | 2978514 | − | | |
| cg01784928 | 77647704 | − | N_Shore | TMEM63C(tss1500) |
| cg26986681 | 58060609 | − | N_Shore | IGFBP7-AS1(body) |
| cg02683408 | 79487159 | − | S_Shore | |
| cg03680291 | 47052148 | + | S_Shelf | |
| cg00003578 | 12600529 | − | Island | |
| cg13779349 | 1.11E+08 | − | S_Shelf | TRPCS(tss1500); ZCCHC16(body) |
| cg04944300 | 17022524 | + | N_Shore | ESPNP(body) |
| cg02838492 | 1.17E+08 | + | Island | KIF12(body) |
| cg21829490 | 74499584 | − | Island | |
| cg25228169 | 72489416 | − | N_Shore | GRAMD2(body) |
| cg24790788 | 1.46E+08 | − | S_Shore | PLOD2(tss1500) |
| cg03931865 | 47048107 | − | Island | |
| cg09669835 | 91510500 | − | S_Shore | MTERF(tss1500) |
| cg11968804 | 2.13E+08 | + | Island | |
| cg21456083 | 49524256 | + | S_Shore | |
| cg24778776 | 69017394 | + | | |
| cg07745707 | 88496476 | − | Island | ZNF469(body) |
| cg22194188 | 62341149 | − | S_Shore | TEX2(tss1500) |
| cg14884373 | 3644023 | + | S_Shore | GFRA4(body) |
| cg14621323 | 1.77E+08 | − | Island | PFN3(body) |
| cg17924225 | 1.72E+08 | − | | |
| cg02680566 | 19559792 | − | N_Shore | OSR1(tss1500) |
| cg14560430 | 32863175 | − | Island | TRIM71(body) |
| cg20254361 | 24438778 | − | | MYOM3(tss200) |
| cg00068428 | 49494724 | + | | |
| cg05554161 | 1.28E+08 | + | Island | OLFML2A(body) |
| cg07655487 | 66849820 | + | | |
| cg02147681 | 17603837 | − | Island | RAI1(body) |
| cg23362884 | 1.12E+08 | − | S_Shore | RBM20(body) |
| cg25181043 | 47013841 | − | Island | |
| cg18669381 | 16539088 | + | | ARHGEF19(body) |
| cg17839399 | 22777409 | − | N_Shore | ETNK1(tss1500) |
| cg24136671 | 1.05E+08 | − | | |
| cg16270485 | 21901260 | + | | ALPL(body) |
| cg00670942 | 74836683 | + | | |
| cg21243717 | 1.79E+08 | − | | |
| cg09759994 | 1.13E+08 | − | N_Shore | TTL(tss1500) |
| cg04353586 | 7039323 | − | | CAMTA1(body) |
| cg16412370 | 45079144 | − | Island | HSF2BP(body); RRP1B(tss1500) |
| cg25875537 | 21909033 | − | S_Shelf | |
| cg03575765 | 1.04E+08 | + | S_Shore | |
| cg06719448 | 1.28E+08 | − | | FAM71F1(body); FAM71F1(tss200) |
| cg10724867 | 27218867 | − | N_Shore | HOXA9(body); HOXA10- |
| | | | | HOXA9(body) |
| cg24166966 | 33319712 | − | S_Shore | FBXL2(body) |
| cg13710556 | 25893925 | + | S_Shore | |
| cg24550343 | 1.81E+08 | − | N_Shore | |
| cg09732145 | 1.97E+08 | − | | |
| cg23369909 | 1.35E+08 | − | Island | |
| cg17828057 | 42037527 | + | | PLAT(body) |
| cg03480346 | 24232603 | − | N_Shore | MFSD2B(tss1500) |
| cg07915343 | 18477680 | − | Island | LOHAL6A(body) |
| cg10831901 | 5485294 | + | | LINC00654(tss200) |
| cg18438319 | 1.55E+08 | + | | |
| cg22200736 | 52995358 | − | S_Shore | KRT72(tss200) |
| cg14809332 | 1.45E+08 | + | S_Shore | MROH6(body) |
| cg26034481 | 97092205 | + | | |
| cg09867953 | 23706666 | + | | ERN2(body) |
| cg21695020 | 68014626 | + | S_Shore | DPEP3(tss200) |
| cg02395590 | 1.41E+08 | − | | |
| cg16731266 | 28601269 | − | N_Shore | |
| cg27629589 | 43295011 | − | N_Shore | |
| cg02109484 | 74875253 | + | Island | M1AP(body); M1AP(tss200) |
| cg05191217 | 1.54E+08 | − | Island | |
| cg04166411 | 25812331 | + | | EBF2(body) |
| cg21377521 | 29744537 | − | | TMTC1(body) |
| cg01781963 | 61028748 | + | | |
| cg20974196 | 859983 | − | N_Shore | CFD(body) |
| cg01435791 | 4585323 | − | S_Shore | |
| cg19908485 | 1.49E+08 | − | Island | NBPF23(tss1500) |
| cg11503274 | 1.5E+08 | + | Island | CDX1(body) |
| cg12244325 | 45883864 | − | Island | |
| cg08013206 | 64345204 | + | | |
| cg09895590 | 1.19E+08 | + | N_Shelf | VAX1(body) |
| cg11395890 | 15712019 | − | | FHAD1(body) |
| cg05046097 | 88717926 | + | | MMRN2(tss1500); SNCG(tss1500) |
| cg11248999 | 23067453 | − | | EPHB2(body) |
| cg07891179 | 29923883 | − | Island | WIPF3(body) |
| cg07226718 | 95841414 | − | | |
| cg18060171 | 13319777 | + | Island | CACNA1A(body) |
| cg14550163 | 1.12E+08 | + | Island | CUX2(body) |
| cg06434451 | 32802291 | − | S_Shore | MARCKSL1(tss1500) |
| cg22804000 | 24797691 | + | S_Shore | DFNA5(tss1500); DFNA5(tss200) |
| cg00406022 | 31691696 | + | Island | C6orf25(body) |
| cg05280814 | 1.34E+08 | − | N_Shelf | |
| cg05585991 | 1.57E+08 | − | S_Shore | C5orf52(body) |
| cg23300529 | 21989012 | + | Island | HR(tss1500) |
| cg09322973 | 8333010 | + | Island | FAM66C(body) |
| cg12586386 | 77299805 | + | N_Shore | AQP11(tss1500) |
| cg04826345 | 22615334 | − | | |
| cg05803853 | 1.26E+08 | + | | |
| cg09933458 | 1.12E+08 | − | Island | |
| cg09256832 | 9382878 | − | | SPSB1(body) |
| cg20089744 | 1.28E+08 | − | S_Shelf | |
| cg16942223 | 6050460 | + | | JAKMIP1(body) |
| cg04412755 | 46939978 | − | N_Shore | PTH1R(body) |
| cg09042678 | 18117663 | − | N_Shore | PET117(tss1500) |
| cg17969123 | 18745971 | + | | |
| cg27494647 | 1.5E+08 | + | Island | RARRES2(tss200) |
| cg04206342 | 70636940 | − | | LINC00511(tss1500) |
| cg08019058 | 1.06E+08 | + | | |
| cg04947136 | 20032627 | − | | |
| cg05077231 | 49793054 | + | N_Shore | MLNR(tss1500) |
| cg13161901 | 59221985 | + | Island | CDH20(body) |
| cg21633052 | 1.84E+08 | − | N_Shore | WWC2-AS2(body) |
| cg21846790 | 4666047 | + | Island | UBALD1(tss1500) |
| cg02206767 | 17990805 | + | N_Shore | DRG2(tss1500) |
| cg06797617 | 88649907 | − | N_Shore | |
| cg24128434 | 7623131 | − | S_Shelf | DNAH2(body) |
| cg02523270 | 1.94E+08 | + | Island | |
| cg01947751 | 1.97E+08 | − | Island | MFI2(body); MFI2-AS1(tss1500) |
| cg19242688 | 47139391 | − | Island | GNG8(tss1500) |
| cg13550419 | 44898510 | − | S_Shore | |
| cg16868095 | 1.97E+08 | + | | |
| cg03623111 | 67430099 | − | S_Shore | ZDHHC1(body) |
| cg25002788 | 1166873 | + | S_Shore | SPON2(body); SPON2(tss1500) |
| cg08971217 | 87086785 | + | | |
| cg13717817 | 57177391 | − | | IL17RD(body) |
| cg12629515 | 27859811 | − | N_Shore | HIST1H2BO(tss1500); HIST1H3J(tss1500) |
| cg19695978 | 70877381 | − | Island | |
| cg01365639 | 50975940 | + | N_Shore | |
| cg18152887 | 920593 | + | | |
| cg01292846 | 33672543 | − | | |
| cg01390950 | 34117273 | − | S_Shelf | GRM4(body) |
| cg07262247 | 1.32E+08 | + | Island | PDLIM4(body) |
| cg16620608 | 1.01E+08 | − | N_Shelf | MOGAT3(body) |
| cg26345105 | 2.29E+08 | − | S_Shore | |
| cg12275060 | 45279329 | − | Island | BTBD19(body) |
| cg02870820 | 56003211 | + | N_Shore | SSC5D(body) |
| cg23146472 | 43214495 | − | Island | TTBK1(body) |
| cg00082140 | 1.8E+08 | + | Island | |
| cg25845955 | 32118405 | − | Island | PRRT1(body) |
| cg13574820 | 1.55E+08 | + | Island | |
| cg03719040 | 4250665 | − | S_Shore | TMEM128(tss1500) |
| cg12584458 | 28447107 | − | | |
| cg17265358 | 781272 | + | S_Shore | CPLX1(body) |
| cg12946440 | 1753573 | − | Island | ELFN1(body) |
| cg05866854 | 1.72E+08 | + | N_Shore | GORASP2(tss200) |
| cg03811829 | 949449 | − | Island | ISG15(body) |
| cg26175729 | 1.51E+08 | − | S_Shore | ABCB8(body) |
| cg06961147 | 1.6E+08 | − | N_Shore | TANC1(tss1500) |
| cg03254146 | 30240119 | − | | |
| cg24158324 | 55525967 | + | S_Shelf | |
| cg26789888 | 1.2E+08 | + | | |
| cg04524088 | 1.28E+08 | + | | MIR129-1(tss200) |
| cg01124539 | 1.32E+08 | + | N_Shore | |
| cg02876276 | 77834299 | − | Island | |
| cg02121529 | 40746549 | + | Island | |
| cg13447295 | 887704 | − | Island | |
| cg14259717 | 18433732 | − | | LDHC(tss200) |
| cg06029792 | 70326224 | − | Island | |
| cg02250764 | 1.35E+08 | − | N_Shore | |
| cg24091438 | 1.5E+08 | − | Island | ACTR3C(body); LRRC61(tss1500) |
| cg06193239 | 68417787 | + | | |
| cg23853507 | 1651391 | − | | ADARB2(body) |
| cg11237792 | 1.78E+08 | − | S_Shore | TTC30A(tss1500) |
| cg03670393 | 81128690 | + | Island | |
| cg13672871 | 27317381 | − | | |
| cg04855401 | 1.3E+08 | + | N_Shore | |
| cg05498866 | 22018549 | + | S_Shelf | SFTPC(tss1500) |
| cg13390800 | 6677919 | − | S_Shore | DCHS1(tss1500) |
| cg05015938 | 1.6E+08 | − | S_Shelf | FNDC1(body) |
| cg14494090 | 1.35E+08 | − | N_Shore | KNDC1(tss1500) |
| cg04648334 | 27236866 | − | | |
| cg11924019 | 75019283 | − | Island | CYP1A1(tss1500) |
| cg09811860 | 2.12E+08 | − | | |
| cg01251360 | 68772225 | + | Island | CDH1(body) |
| cg04375683 | 52692431 | + | N_Shore | PPP2R1A(tss1500) |
| cg07139196 | 30976950 | + | S_Shore | |
| cg26883837 | 46798244 | − | N_Shore | |
| cg07954075 | 1633258 | − | | |
| cg22867608 | 64573356 | − | Island | EGR2(body) |
| cg02698886 | 24360603 | − | S_Shore | DCDC2(body) |
| cg18755942 | 1.39E+08 | − | N_Shore | C3orf72(body); FOXL2(tss1500) |
| cg09648727 | 79361742 | − | S_Shore | |
| cg16260421 | 50489319 | + | N_Shore | AGBL4(body) |
| cg07014308 | 31691597 | + | Island | C6orf25(body) |
| cg18417954 | 55672513 | + | Island | DNAAF3(body) |
| cg10754705 | 22085381 | + | N_Shelf | PHYHIP(body) |
| cg05142520 | 49364426 | − | Island | WNT10B(body) |
| cg08044427 | 20442131 | − | | |
| cg12716837 | 7518360 | − | | SORCS2(body) |
| cg14315430 | 57915618 | − | N_Shore | DDIT3(tss1500); MBD6(tss1500) |
| cg02798848 | 1.55E+08 | + | | |
| cg01923075 | 1.05E+08 | − | Island | |
| cg06629734 | 39017116 | + | S_Shore | GLP1R(body) |
| cg27615366 | 1.32E+08 | − | N_Shore | PDLIM4(tss1500) |
| cg19211931 | 1.15E+08 | − | N_Shore | DPP10(body) |
| cg17793372 | 62179030 | + | Island | SRMS(tss200) |
| cg11468148 | 670815 | − | S_Shore | DIP2C(body) |
| cg16777618 | 1.29E+08 | − | Island | |
| cg25213362 | 51236634 | − | Island | TMPRSS12(tss200) |
| cg10366786 | 53578129 | + | N_Shore | DANCR(tss1500); MIR4449(tss1500); |
| | | | | SNORA26(tss1500) |
| cg05051605 | 22103855 | − | | VWA3A(tss200) |
| cg08142858 | 2718742 | + | S_Shore | |
| cg04252762 | 1.65E+08 | + | S_Shelf | |
| cg26334166 | 61950840 | + | N_Shore | COL20A1(body) |
| cg26003909 | 1.43E+08 | − | | |
| cg01146943 | 51416749 | − | N_Shore | DLEU7(body); DLEU7-AS1(body) |
| cg19260189 | 1.07E+08 | − | | LOC100287944(body); RFX4(body) |
| cg23474811 | 4183807 | + | | SDK1(body) |
| cg18686665 | 629121 | + | Island | |
| cg24438909 | 1.57E+08 | + | N_Shore | INSRR(body); NTRK1(body) |
| cg20322862 | 3412088 | − | | TGIF1(body); TGIF1(tss1500) |
| cg26192556 | 1.02E+08 | − | S_Shore | SLCO6A1(tss200) |
| cg16807687 | 85974018 | − | | CDHR1(body) |
| cg07018645 | 75108826 | − | | LMAN1L(body) |
| cg04580443 | 1.16E+08 | + | | |
| cg26474549 | 2.39E+08 | + | | LINC01107(body) |
| cg23239444 | 34433320 | − | N_Shore | PACSIN1(tss1500) |
| cg05845831 | 2.32E+08 | − | N_Shelf | |
| cg03076771 | 35233484 | + | N_Shore | C20orf24(tss1500); TGIF2- |
| | | | | C20orf24(body) |
| cg03305746 | 1.07E+08 | − | | |
| cg02159289 | 39967202 | + | Island | BCOR(body) |
| cg14495399 | 1.25E+08 | + | | |
| cg21074766 | 99069077 | + | | |
| cg02376887 | 29542219 | − | Island | OVCH1-AS1(tss200) |
| cg05098551 | 1.79E+08 | + | N_Shore | FKBP7(tss1500); MIR548N(body); PL6KHA3 |
| | | | | (tss1500) |
| cg06225242 | 1.25E+08 | − | | |
| cg03748603 | 2494443 | + | Island | |
| cg03015592 | 17602053 | + | | OTOG(body) |
| cg26390598 | 41032396 | − | | B3GALT5(body) |
| cg00383763 | 2.03E+08 | + | S_Shelf | |
| cg23405152 | 1.21E+08 | + | Island | |
| cg14562060 | 35109298 | + | Island | TCP11(tss200) |
| cg18754388 | 33132637 | + | S_Shelf | COL11A2(body) |
| cg27419075 | 72295168 | − | Island | PDE2A(body) |
| cg07845392 | 79678158 | − | N_Shore | SLC25A10(tss1500) |
| cg18411150 | 51172144 | − | S_Shore | SHANK1(body) |
| cg07769947 | 2.21E+08 | − | | |
| cg01687862 | 75814979 | − | | |
| cg13516209 | 29855302 | + | Island | HLA-H(tss1500) |
| cg19002763 | 21910390 | + | N_Shelf | DMTN(tss1500) |
| cg01948047 | 19724501 | + | Island | |
| cg18402261 | 48021977 | − | | |
| cg21298759 | 24741723 | − | N_Shore | NIPAL3(tss1500); STPG1(tss1500); STPG1 |
| | | | | TPG1(tss200) |
| cg03006527 | 1.55E+08 | − | S_Shore | |
| cg09263059 | 43065876 | − | | |
| cg04095097 | 15515112 | + | | |
| cg18031880 | 1.16E+08 | − | | |
| cg14222229 | 1.49E+08 | − | Island | NBPF23(body) |
| cg01147352 | 86767299 | − | | |
| cg27309611 | 1.32E+08 | − | S_Shore | SEPT8(tss1500); SEPT8(tss200) |
| cg09900722 | 43838112 | − | Island | |
| cg09120934 | 1.2E+08 | − | | POU2F3(body) |
| cg00945260 | 1104925 | − | N_Shelf | |
| cg03893692 | 1.5E+08 | + | | CAMK2A(tss200) |
| cg03070187 | 6739184 | + | N_Shore | TRIP10(tss1500) |
| cg22704845 | 1.03E+08 | + | N_Shore | |
| cg25004981 | 1.01E+08 | + | Island | CERS3(tss200) |
| cg27113326 | 1.57E+08 | − | Island | PTPRN2(body) |
| cg03669698 | 29707524 | + | Island | GAS2L1(body) |
| cg26679004 | 88023135 | − | Island | GRID1(body) |
| cg05399320 | 20623913 | − | S_Shore | SLC6A5(body) |
| cg21917728 | 41471064 | + | N_Shore | |
| cg13095450 | 35423449 | − | | |
| cg11580525 | 1.18E+08 | − | N_Shore | SCN4B(body) |
| cg21221545 | 9286050 | − | | |
| cg27438425 | 41952221 | − | | |
| cg00843634 | 10584169 | + | Island | SOX7(body) |
| cg14663607 | 2.2E+08 | − | N_Shelf | WNT10A(body) |
| cg06187336 | 1.45E+08 | − | Island | MAFA(tss1500) |
| cg23170347 | 64253795 | − | Island | |
| cg01596963 | 62455830 | − | Island | LRRN4CL(body) |
| cg10486974 | 34499389 | − | Island | PACSIN1(body) |
| cg27217253 | 8648562 | − | N_Shore | CCDC42(tss1500) |
| cg04650834 | 49456262 | + | | |
| cg16900255 | 74037743 | + | | C15orf59(body) |
| cg19408398 | 54243328 | + | N_Shore | FIPIL1(tss1500) |
| cg23830810 | 1.21E+08 | + | S_Shore | COQS(tss1500) |
| cg10554436 | 1.39E+08 | + | N_Shore | C3orf72(body):FOXL2(tss1500) |
| cg19478079 | 79453653 | + | N_Shore | |
| cg01320298 | 15160158 | − | | |
| cg11405475 | 2.04E+08 | − | N_Shore | SNRPE(tss1500) |
| cg12116535 | 1.39E+08 | + | S_Shore | MCF2(body) |
| cg13910377 | 43111998 | − | S_Shelf | LINC00111(body) |
| cg22273182 | 1.02E+08 | − | Island | |
| cg07160871 | 2.22E+08 | + | | |
| cg01489256 | 11204017 | − | N_Shore | TDH(body) |
| cg23289456 | 749620 | + | | SLC52A3(tss1500) |
| cg13560058 | 1097233 | + | N_Shore | |
| cg26580609 | 18766140 | − | Island | NT5C1B(body); NT5C1B- |
| | | | | RDH14(body) |
| cg21995147 | 1064017 | − | Island | ABCA7(body) |
| cg15546638 | 93277793 | + | | |
| cg16574155 | 27287468 | − | S_Shore | C1orf172(tss1500) |
| cg12499062 | 11519034 | − | | |
| cg07511014 | 1.35E+08 | + | Island | PAOX(tss1500) |
| cg14688662 | 1688174 | + | S_Shore | |
| cg06653203 | 13860477 | + | Island | WNT7A(body) |
| cg18331412 | 1.35E+08 | + | Island | TGFBI(body) |
| cg11437223 | 35078097 | − | S_Shore | DPY19L1(tss1500) |
| cg16091553 | 46126245 | + | | GABRG1(tss200) |
| cg26228746 | 13276279 | − | | LICMA(body) |
| cg09269848 | 1396074 | + | S_Shore | MYO1C(tss1500); MYO1C(tss200) |
| cg19360482 | 1726171 | + | Island | |
| cg01195404 | 467135 | + | N_Shore | |
| cg01446692 | 14723496 | − | | CER1(tss1500) |
| cg11615607 | 1.2E+08 | − | S_Shore | |
| cg01120874 | 1.34E+08 | − | N_Shore | FIBCD1(body) |
| cg06983508 | 1.25E+08 | + | N_Shore | SLC12A8(body); SLC12A8(tss1500) |
| cg14607660 | 36738765 | − | N_Shore | |
| cg24387126 | 11617293 | − | | C8orf49(tss1500); GATA4(body) |
| cg10552903 | 1.22E+08 | − | | BCL7A(tss1500) |
| cg26973074 | 1.18E+08 | + | S_Shore | KIAA1210(tss1500) |
| cg24222546 | 74501771 | + | | STRA6(body); STRA6(tss1500) |
| cg01916632 | 1.08E+08 | − | S_Shore | PDSS2(tss1500) |
| cg11086695 | 98335201 | + | | |
| cg22973459 | 1.03E+08 | + | | |
| cg24682986 | 11101247 | − | Island | |
| cg13359765 | 2.34E+08 | + | S_Shore | SLC35F3(body) |
| cg18452703 | 1.03E+08 | − | | |
| cg13032057 | 30922803 | − | N_Shore | |
| cg18435000 | 249109 | + | S_Shore | IQSEC3(body); LOC574538(body) |
| cg06632797 | 68968215 | − | | |
| cg27543040 | 2.48E+08 | − | N_Shore | |
| cg04912710 | 72460643 | + | S_Shore | |
| cg06795125 | 1.09E+08 | − | | SLILT1C2(body) |
| cg01134643 | 5406948 | − | | LICN3(tss200) |
| cg08633479 | 57630524 | + | Island | LISP29(tss1500) |
| cg26164234 | 98946778 | − | S_Shore | ARHGAP19- |
| | | | | SLIT1(body); SLIT1(tss1500) |
| cg09637273 | 55939387 | + | Island | CLIEDC1(body) |
| cg02007434 | 98210804 | + | | TLL2(body) |
| cg12705353 | 1.22E+08 | + | Island | WDR66(body) |
| cg20704560 | 10698313 | + | Island | CASZ1(body) |
| cg14016344 | 35616726 | − | N_Shore | CD72(body) |
| cg17186388 | 29923435 | − | N_Shore | WIPF3(body) |
| cg13708645 | 1.22E+08 | − | N_Shore | KDM2B(body) |
| cg22979368 | 2.18E+08 | + | S_Shelf | IGFBP2(body) |
| cg00115747 | 74940026 | + | | MGAT5B(body) |
| cg09230763 | 27683351 | − | Island | MAP3K6(body) |
| cg01283343 | 42211160 | + | | CEACAM5(tss1500) |
| cg06414816 | 1.1E+08 | − | | TMEM232(tss1500) |
| cg00253658 | 54210496 | − | | |
| cg07832006 | 1.15E+08 | + | N_Shore | SYCP1(tss200) |
| cg09359017 | 39207132 | + | | |
| cg17840408 | 396686 | − | Island | PKP3(body) |
| cg16289210 | 2.03E+08 | − | | FMOD(body) |
| cg04202736 | 42371721 | + | Island | PLA2G4D(body) |
| cg26100214 | 8580376 | + | N_Shore | |
| cg17211562 | 11886253 | − | Island | LPIN1(body); LPIN1(tss1500) |
| cg25097610 | 4763360 | − | | |
| cg10043663 | 2.2E+08 | − | N_Shelf | ASIC4(body) |
| cg19904265 | 3166712 | + | S_Shelf | PRDM16(body) |
| cg24122922 | 24449392 | − | N_Shore | SYNDIG1(tss1500) |
| cg02356435 | 16925533 | − | Island | PLCL2(tss1500) |
| cg00422578 | 1.2E+08 | + | | |
| cg14021880 | 27301369 | − | N_Shelf | EMILIN1(tss200) |
| cg22856010 | 1707413 | − | N_Shelf | |
| cg24730688 | 21023823 | − | S_Shore | KIF17(body) |
| cg17301379 | 1.35E+08 | + | Island | TTC40(body) |
| cg18782651 | 65969785 | − | N_Shore | |
| cg18748499 | 38517641 | − | N_Shore | GJD3(body) |
| cg13504318 | 17932100 | + | Island | INSL3(body) |
| cg04802386 | 1.04E+08 | − | S_Shore | SLC9B2(tss200) |
| cg02962521 | 19744694 | − | N_Shore | |
| cg07111415 | 1.96E+08 | + | S_Shelf | ZDHHC19(tss200) |
| cg23800435 | 43751376 | − | | C1orf210(tss200) |
| cg01643605 | 29040623 | + | | |
| cg07464674 | 6358363 | + | N_Shore | PITPNM3(body) |
| cg18530107 | 93311962 | + | | LOC100188947(body) |
| cg23586996 | 77893850 | − | | |
| cg02989257 | 32169274 | − | N_Shore | COL16A1(body) |
| cg12923268 | 31691812 | − | S_Shore | C6orf25(body) |
| cg04993130 | 726759 | − | Island | EPS8L2(body) |
| cg23708717 | 47793175 | + | N_Shore | CCDC11(tss1500) |
| cg19164987 | 54413000 | + | Island | HOXC4(body); HOXC5(body); HOXC6(body) |
| cg14910288 | 10033220 | − | S_Shore | |
| cg13799544 | 29432016 | − | | FAM189A1(body) |
| cg13993218 | 2181866 | − | | INS(body); INS-IGF2(body) |
| cg18901140 | 8908225 | + | S_Shore | |
| cg03719509 | 77945178 | + | Island | LHFPL2(tss1500) |
| cg13218688 | 72125544 | − | S_Shore | FAM69C(tss1500) |
| cg22929099 | 1.38E+08 | + | Island | GFRA3(body) |
| cg11004890 | 3218500 | + | N_Shore | SLC4A11(body); SLC4A11(tss200) |
| cg09593454 | 3439372 | + | S_Shore | TRPV3(body) |
| cg15841205 | 37056458 | − | S_Shelf | |
| cg23596567 | 11901585 | + | S_Shore | ZNF18(tss1500) |
| cg25316749 | 1.11E+08 | + | N_Shore | COLCA1(body); COLCA2(body); COLCA2(tss1500) |
| cg18452973 | 3446004 | + | | |
| cg21535580 | 2.04E+08 | − | S_Shore | ETNK2(tss1500) |
| cg27384088 | 72298275 | − | Island | |
| cg07752409 | 1.8E+08 | + | S_Shore | RASGEF1C(body) |
| cg02991085 | 30073537 | + | S_Shore | LINC00028(tss200) |
| cg18758664 | 90320475 | + | Island | MESP2(body) |
| cg14968482 | 45938585 | + | N_Shore | ALOX5(body) |
| cg22896075 | 38518413 | + | N_Shore | GJD3(body) |
| cg13215995 | 43222258 | − | Island | |
| cg12285565 | 26047780 | − | N_Shore | |
| cg02439077 | 72202215 | − | S_Shore | NODAL(tss1500) |
| cg08141989 | 7082562 | − | | ASGR1(body) |
| cg21827317 | 1.37E+08 | − | S_Shore | |
| cg10333023 | 1.29E+08 | + | | C11orf45(body); KCNJ5(body) |
| cg26615126 | 3642867 | + | S_Shore | COLEC11(body) |
| cg02924487 | 3649344 | + | Island | TP73(body) |
| cg03848890 | 26782564 | − | N_Shelf | OTOF(tss1500) |
| cg24059075 | 49688316 | − | N_Shore | PRPH(tss1500) |
| cg13451000 | 60383256 | + | | LINC00301(body) |
| cg08533674 | 46993347 | + | | |
| cg26312807 | 1.27E+08 | − | Island | |
| cg00851377 | 1.13E+08 | + | | GPR85(body) |
| cg17743381 | 39024825 | + | | |
| cg11830761 | 1.26E+08 | − | Island | |
| cg11490745 | 2.23E+08 | − | N_Shelf | PAX3(body) |
| cg03711791 | 49626347 | + | Island | KCNG1(body) |
| cg20544552 | 85393693 | + | | |
| cg00451635 | 10675030 | − | S_Shore | EMP2(tss1500) |
| cg01925738 | 1.4E+08 | + | N_Shore | PCDHB3(body) |
| cg26038649 | 27683501 | + | Island | MAP3K6(body) |
| cg00758881 | 58534681 | − | N_Shore | NDRG4(body) |
| cg11813441 | 1.77E+08 | + | N_Shore | |
| cg06246950 | 31030853 | − | N_Shore | SLC35E4(tss1500) |
| cg04828580 | 93639778 | + | | |
| cg17338254 | 19752522 | − | N_Shore | TBX1(body) |
| cg14632490 | 46911983 | − | N_Shore | |
| cg22841667 | 8705620 | + | S_Shore | RPL27A(body); SNORA3(tss200); SNORA45 |
| | | | | (tss1500) |
| cg01922437 | 67461690 | + | N_Shore | |
| cg10239319 | 6073656 | − | | JAKMIP1(body) |
| cg23819145 | 24583953 | − | S_Shore | ITSN2(tss1500) |
| cg10938556 | 1.05E+08 | − | Island | |

TABLE 2
Comparison between different prediction algorithms and NSD1 +/− DNAm signature for missense mutations

| | | Pmut | Pmut | Polyphen-2 | | | | | NSD1 | |
|---|---|---|---|---|---|---|---|---|---|---|
| | Protein | Prediction | Prediction | PolyPhen- | prediction | | | Mut- | DNAm | |
| Sample ID | Change | (reliability) | probability | 2 score | effect | Mutation Taster | SIFT | Pred* | signature | Inheritance |
| HK-5474 | C1606Y | Pathological (9) | 0.9844 | 1 | Probably | Disease causing | Deleterious | 0.794 | yes | De novo |
| | | | | | damaging | (p-value: 1) | (score: 0) | | | |
| HK-11693 | P1726R | Pathological (7) | 0.895 | 1 | Probably | Disease causing | Deleterious | 0.82 | yes | De novo |
| | | | | | damaging | (p-value: 1) | (score: 0) | | | |
| HK-5581 | V1968A | Neutral (1) | 0.4222 | 1 | Probably | Disease causing | Deleterious | 0.703 | yes | De novo |
| | | | | | damaging | (p-value: 1) | (score: 0) | | | |
| HK-3326 | Y1997C | Pathological (8) | 0.9346 | 1 | Probably | Disease causing | Deleterious | 0.965 | yes | De novo |
| | | | | | damaging | (p-value: 1) | (score: 0.04) | | | |
| HK-435 | R2017W | Pathological (9) | 0.9916 | 1 | Probably | Disease causing | Deleterious | 0.971 | yes | De novo |
| | | | | | damaging | (p-value: 1) | (score: 0.04) | | | |
| DL136303 | A1927P | Pathological (3) | 0.6663 | 1 | Probably | Disease causing | Deleterious | 0.674 | yes | Unknown |
| | | | | | damaging | (p-value: 1) | (score: 0) | | | |
| DL208122 | C2146S | Pathological (1) | 0.5943 | 1 | Probably | Disease causing | Deleterious | 0.983 | yes | De novo |
| | | | | | damaging | (p-value: 1) | (score: 0) | | | |
| DL199861 | C2138R | Pathological (8) | 0.949 | 1 | Probably | Disease causing | Deleterious | 0.944 | yes | De novo |
| | | | | | damaging | (p-value: 1) | (score: 0) | | | |
| DL159025 | R2005G | Pathological (6) | 0.8303 | 1 | Probably | Disease causing | Deleterious | 0.761 | yes | De novo |
| | | | | | damaging | (p-value: 1) | (score: 0) | | | |
| DL181344 | N1650S | Pathological (0) | 0.5007 | 0 | Benign | Disease causing | Tolerated | 0.65 | no | De novo |
| | | | | | | (p-value: 0.943) | (score: 0.68) | | | |
| HK-9776 | N357S | Neutral (5) | 0.2237 | 0.103 | Benign | Polymorphism | Tolerated | 0.462 | no | Mat. |
| | | | | | | (p-value: 0.957) | (score: 0.25) | | | Inheritance |
| HK-12366 | N1149S | Neutral (2) | 0.3611 | 0.001 | Benign | Polymorphism | Tolerated | 0.101 | no | De novo |
| | | | | | | (p-value: 1) | (score: 0.28) | | | |
| HK-6943 | P2225Q | Neutral (0) | 0.4854 | 0.003 | Benign | Polymorphism | Tolerated | 0.232 | no | Mat. |
| | | | | | | (p-value: 0.988) | (score: 0.14) | | | Inheritance |
| HK-14867 | Q2474R | Neutral (1) | 0.4022 | 0 | Benign | Polymorphism | Deleterious | 0.178 | no | De novo |
| | | | | | | (p-value: 1) | (score: 0.01) | | | |
| HK-11767 | K1786R | Neutral (8) | 0.0537 | 0.236 | Benign | Disease causing | Deleterious | 0.537 | no | Mat. |
| | | | | | | (p-value: 0.982) | (score: 0) | | | Inheritance |
| DL73286 | S1241T | Neutral (8) | 0.0911 | 0 | Benign | Polymorphism | Tolerated | 0.044 | no | Mat. |
| | | | | | | (p-value: 1) | (score: 0.14) | | | Inheritance |

TABLE 3
Sotos patients with NSD1 (NM_022455.4, NCBI hg18) loss
of function mutations-Replication study

| Sample ID | Exon | Mutation | Protein Change | Gender |
|---|---|---|---|---|
| Whole gene | | | | |
| deletions | | | | |
| HK-1540 | | WGD | | M |
| HK-5107 | | WGD | | F |
| HK-15164 | | WGD | | F |
| HK-15438 | | WGD | | F |
| Intragenic | | | | |
| deletions | | | | |
| HK-5533 | 5 | c.2314delG | p.Ala772GInfs*19 | M |
| HK-5439 | 5 | c.3728delG | p.Ser1243Thrfs*14 | F |
| HK-7783 | 8 | c.4217delGAAA | p.Arg1406Asnfs*12 | M |
| Insertion | | | | |
| HK-5530 | 5 | c.2334_c.2335insA | p.His779Thrfs*30 | M |
| HK-13565 | 5 | c.2607_c.2608insA | p.Arg870Lysfs*5 | F |
| HK-11302 | 16 | c.5397_c.5398insT | p.Gly1800Trpfs*3 | F |
| Nonsense | | | | |
| Mutation | | | | |
| HK-4913 | 5 | c.2362C > T | p.Arg788* | F |
| HK-5589 | 5 | c.1310C > G | p.Ser437* | F |
| HK-4470 | 5 | c.1754C > G | p.Ser585* | F |
| HK-6779 | 5 | c.3067C > T | p.Arg1023* | M |
| HK-2129 | 10 | c.4417C > T | p.Arg1473* | F |
| HK-6966 | 15 | c.5296C > A | R1766X p.= | F |
| | | | (p.Arg1766Arg) | |
| HK-11807 | 18 | c.5887A > T | p.Lys1963* | F |
| HK-10067 | 20 | c.6013C > T | p.Arg2005* | M |
| HK-5977 | 22 | c.6349C > T | p.Arg2117* | F |

TABLE 4
Weaver patients with EZH2 (NM_004456.4, NCBI hg19)
mutations/deletions

| Sample ID | Exon | Mutation | Protein change | Gender |
|---|---|---|---|---|
| A123 | 6 | c.457_459del | p.Tyr153del | M |
| A134 | 19 | c.2080C > T | p.His694Tyr | F |
| A117 | 6 | c.394C > T | p.Pro132Ser | F |
| A120 | 6 | c.394C > T | p.Pro132Ser | M |
| A212 | 6 | c.398A > G | p.Tyr133Cys | F |
| A1359 | 19 | c.2050C > T | p.Arg684Cys | M |
| F0123 | 7 | c.553G > C | p.Asp185His | M |
| DL129940 | 16 | c.1876G > A | p.Val626Met | M |

TABLE 5
Summary of Sensitivity and Specificity at Various Delta-Beta Cutoff Values.

| | | Unique | | | | | | |
|---|---|---|---|---|---|---|---|---|
| Delta-beta | num_CpGs | Genes | Specificity_GEO | Specificity_LOO | Sensitivity_LOO | Precision_LOO | Recall_LOO | F-measure_LOO |
| 0.4 | 726 | 275 | 100.00% | 100.00% | 100.00% | 100.00% | 100.00% | 100.00% |
| 0.42 | 549 | 212 | 100.00% | 100.00% | 100.00% | 100.00% | 100.00% | 100.00% |
| 0.44 | 399 | 164 | 100.00% | 100.00% | 100.00% | 100.00% | 100.00% | 100.00% |
| 0.46 | 287 | 127 | 99.62% | 100.00% | 100.00% | 100.00% | 100.00% | 100.00% |
| 0.48 | 220 | 101 | 99.24% | 100.00% | 100.00% | 100.00% | 100.00% | 100.00% |
| 0.5 | 156 | 72 | 96.21% | 100.00% | 100.00% | 100.00% | 100.00% | 100.00% |
| 0.52 | 124 | 67 | 95.64% | 100.00% | 100.00% | 100.00% | 100.00% | 100.00% |
| 0.54 | 87 | 49 | 93.47% | 100.00% | 97.37% | 100.00% | 97.37% | 98.67% |
| 0.56 | 57 | 33 | 88.35% | 98.11% | 94.74% | 97.30% | 94.74% | 96.00% |
| 0.58 | 41 | 24 | 79.55% | 92.45% | 94.74% | 90.00% | 94.74% | 92.31% |
| 0.6 | 25 | 18 | 67.52% | 83.02% | 92.11% | 79.55% | 92.11% | 85.37% |
| 0.62 | 17 | 17 | 68.09% | 79.25% | 73.68% | 71.79% | 73.68% | 72.73% |
| 0.64 | 10 | 12 | 77.27% | 62.26% | 63.16% | 54.55% | 63.16% | 58.54% |
| 0.66 | 7 | 6 | 63.54% | 62.26% | 71.05% | 57.45% | 71.05% | 63.53% |

TABLE 6
List of Weaver subjects with EZH2+/−

| Sample ID | Exon | Mutation | Protein change | Gender |
|---|---|---|---|---|
| A123 | 6 | c.457_459del | p.Tyr153del | M |
| A134 | 19 | c.2080C > T | p.His694Tyr | F |
| A117 | 6 | c.394C > T | p.Pro132Ser | F |
| A212 | 6 | c.398A > G | p.Tyr133Cys | F |
| A1359 | 19 | c.2050C > T | p.Arg684Cys | M |
| F0123 | 7 | c.553G > C | p.Asp185His | M |
| DL129940 | 16 | c.1876G > A | p.Val626Met | M |

TABLE 7
CLinVAr analysis of 3 EZH2 variants

| | | | Clinical |
|---|---|---|---|
| | | | significance |
| Name | Gene | Condition | (Last reviewed) |
| NM_004456.4(EZH2): | EZH2 | not specified\|not | Benign/Likely |
| c.553G > C | | provided | benign (Last |
| (p.Asp185His) | | | reviewed: Mar. |
| | | | 11, 2014) |
| NM_004456.4(EZH2): | EZH2 | Weaver | Pathogenic/Likely |
| c.3940C > T | | syndrome | pathogenic (Last |
| (p.Pro132Ser) | | | reviewed: May 2, |
| | | | 2013) |
| NM_004456.4(EZH2): | EZH2 | Weaver | Pathogenic/Likely |
| c.2080C > T | | syndrome | pathogenic (Last |
| (p.His694Tyr) | | | reviewed: May 2, |
| | | | 2013) |

TABLE 8
Two group comparison by Treatment: Control-blood and Weaver-blood.
p value <0.000377961 and q value (Benjamini correction) <0.0499.

| | | | q-value | | | | | | Genomic | |
|---|---|---|---|---|---|---|---|---|---|---|
| Illumina | Gene | | (Benjamini | | | Control | Weaver | | Location | |
| ID | Symbol | p-value | correction) | Difference | Fold change | Average | Average | Chromosome | (hg19) | Strand |
| cg00969405 | HOXA5 | 1.57E−07 | 0.000330112 | 0.2105 | 1.15709 | 0.024848109 | −0.184586143 | 7 | 27184441 | − |
| cg01323381 | HOXA5 | 1.95E−08 | 7.03E−05 | 0.204768 | 1.1525 | 0.024171429 | −0.179559457 | 7 | 27184264 | − |
| cg01370449 | HOXA5 | 7.07E−10 | 7.61E−06 | 0.288057 | 1.22099 | 0.034003156 | −0.252594957 | 7 | 27183369 | + |
| cg01693350 | WT1 | 7.65E−08 | 0.000200212 | 0.171984 | 1.12661 | 0.020301476 | −0.150811211 | 11 | 32452187 | + |
| cg02005600 | HOXA5 | 2.00E−08 | 7.13E−05 | 0.278755 | 1.21315 | 0.032905243 | −0.244438286 | 7 | 27183686 | − |
| cg02248486 | HOXA5 | 1.47E−10 | 2.22E−06 | 0.338944 | 1.26483 | 0.040010026 | −0.297217557 | 7 | 27183196 | − |
| cg02646423 | HOXA5 | 9.96E−08 | 0.000244389 | 0.227959 | 1.17118 | 0.026909082 | −0.199895857 | 7 | 27183794 | − |
| cg02916332 | HOXA5 | 1.38E−08 | 5.70E−05 | 0.255233 | 1.19353 | 0.030128516 | −0.223811857 | 7 | 27183591 | + |
| cg03368099 | HOXA5 | 5.05E−06 | 0.003844626 | 0.203784 | 1.15172 | 0.024055358 | −0.178696857 | 7 | 27184521 | − |
| cg04236639 | N/A | 2.64E−15 | 5.61E−10 | 0.172506 | 1.12701 | 0.020363251 | −0.151269186 | 21 | 34402565 | − |
| cg04863892 | HOXA5 | 6.60E−09 | 3.37E−05 | 0.342443 | 1.2679 | 0.040423126 | −0.300286286 | 7 | 27183375 | − |
| cg05076221 | HOXA5 | 2.61E−07 | 0.000483974 | 0.215753 | 1.16131 | 0.025468182 | −0.189192186 | 7 | 27182637 | + |
| cg05579037 | N/A | 4.53E−08 | 0.000128088 | 0.17508 | 1.12903 | 0.020666863 | −0.153526129 | 7 | 27184853 | − |
| cg05835726 | HOXA5 | 4.47E−07 | 0.000732328 | 0.248084 | 1.18763 | 0.029284696 | −0.217543471 | 7 | 27183861 | − |
| cg06423211 | N/A | 0.000336 | 0.046718485 | 0.182817 | 1.1351 | 0.021580235 | −0.160310771 | 19 | 551401 | − |
| cg06889571 | ADARB2 | 4.09E−06 | 0.003333145 | 0.172713 | 1.12718 | 0.020387649 | −0.151450617 | 10 | 1416791 | − |
| cg08070327 | HOXA5 | 5.36E−11 | 1.20E−06 | 0.202311 | 1.15054 | 0.023881414 | −0.177404843 | 7 | 27184059 | − |
| cg08787968 | WT1 | 8.24E−09 | 3.93E−05 | 0.175647 | 1.12947 | 0.020733941 | −0.154023443 | 11 | 32451777 | − |
| cg09343092 | HOXA6 | 4.94E−09 | 2.72E−05 | 0.181784 | 1.13429 | 0.021458434 | −0.159404757 | 7 | 27185282 | − |
| cg09549073 | HOXA5 | 2.48E−05 | 0.010370626 | 0.266308 | 1.20273 | 0.031435791 | −0.233523571 | 7 | 27183274 | + |
| cg09644806 | N/A | 5.54E−05 | 0.017432768 | 0.240858 | 1.1817 | 0.028431575 | −0.211207001 | 16 | 85242503 | − |
| cg11724970 | HOXA5 | 5.88E−07 | 0.000892339 | 0.225539 | 1.16921 | 0.026623294 | −0.197773164 | 7 | 27182493 | − |
| cg11985480 | LOC151534 | 3.05E−11 | 8.09E−07 | 0.208387 | 1.1554 | 0.024598706 | −0.182733414 | 2 | 74729100 | + |
| cg12015737 | HOXA5 | 4.02E−12 | 2.44E−07 | 0.172378 | 1.12691 | 0.020347992 | −0.151156486 | 7 | 27184030 | − |
| cg12128839 | HOXA5 | 2.09E−08 | 7.26E−05 | 0.349833 | 1.27441 | 0.041295445 | −0.306765571 | 7 | 27183436 | − |
| cg13512268 | HOXA5 | 7.73E−08 | 0.000200212 | 0.17444 | 1.12853 | 0.020591567 | −0.152965671 | 7 | 27183973 | − |
| cg14013695 | HOXA5 | 3.87E−11 | 9.67E−07 | 0.216377 | 1.16181 | 0.025541924 | −0.189739671 | 7 | 27184176 | − |
| cg14014955 | HOXA5 | 3.64E−09 | 2.21E−05 | 0.270304 | 1.20606 | 0.031907518 | −0.237027843 | 7 | 27183701 | + |
| cg14459158 | N/A | 5.43E−05 | 0.017288631 | 0.174037 | 1.12821 | 0.020543839 | −0.152611529 | 9 | 96720562 | + |
| cg14658493 | HOXA5 | 2.69E−08 | 8.70E−05 | 0.229927 | 1.17278 | 0.027141251 | −0.201621857 | 7 | 27184077 | − |
| cg14882265 | HOXA5 | 1.13E−07 | 0.000264776 | 0.228605 | 1.1717 | 0.026985314 | −0.200462 | 7 | 27184375 | + |
| cg17432857 | HOXA5 | 2.07E−07 | 0.000414667 | 0.202434 | 1.15064 | 0.023896042 | −0.177513143 | 7 | 27184438 | − |
| cg17569124 | HOXA5 | 1.20E−07 | 0.000272781 | 0.297671 | 1.22916 | 0.035138086 | −0.261025857 | 7 | 27183643 | − |
| cg19092981 | TBX1 | 1.64E−08 | 6.21E−05 | 0.361502 | 1.28476 | 0.042672883 | −0.316998571 | 22 | 19751654 | + |
| cg19185846 | N/A | 9.82E−13 | 7.95E−08 | 0.16762 | 1.1232 | 0.019786346 | −0.146984457 | 2 | 71123233 | − |
| cg19759481 | HOXA5 | 6.19E−09 | 3.24E−05 | 0.355488 | 1.27942 | 0.04196298 | −0.311724286 | 7 | 27183401 | − |
| cg20467658 | LOC254559 | 2.79E−07 | 0.000507012 | 0.192027 | 1.14237 | 0.022667583 | −0.168387 | 15 | 89933347 | − |
| cg20517050 | HOXA5 | 1.62E−08 | 6.21E−05 | 0.304726 | 1.23518 | 0.035970864 | −0.267212286 | 7 | 27183806 | − |
| cg20817131 | HOXA5 | 3.70E−09 | 2.21E−05 | 0.255909 | 1.19409 | 0.030208273 | −0.224404343 | 7 | 27184167 | − |
| cg20974609 | HOXA5 | 3.05E−09 | 1.99E−05 | 0.237125 | 1.17864 | 0.027991051 | −0.207933243 | 7 | 27181671 | − |
| cg21010202 | N/A | 8.08E−09 | 3.90E−05 | 0.180893 | 1.13359 | 0.021353194 | −0.158623586 | 6 | 1615843 | + |
| cg21641458 | HOXA6 | 9.62E−09 | 4.35E−05 | 0.182958 | 1.13521 | 0.021597049 | −0.160434614 | 7 | 27185136 | + |
| cg23129930 | HOXA6 | 2.85E−07 | 0.000514811 | 0.203914 | 1.15182 | 0.024070694 | −0.178810814 | 7 | 27186993 | + |
| cg23204968 | HOXA5 | 2.16E−11 | 7.07E−07 | 0.282305 | 1.21614 | 0.033324253 | −0.2475508 | 7 | 27183816 | − |
| cg23454797 | HOXA5 | 2.14E−08 | 7.39E−05 | 0.171518 | 1.12624 | 0.020246492 | −0.150402586 | 7 | 27183990 | + |
| cg23936031 | HOXA5 | 4.49E−10 | 5.29E−06 | 0.306625 | 1.23681 | 0.036194933 | −0.268877114 | 7 | 27183133 | + |
| cg24040595 | HOXA6 | 4.40E−09 | 2.52E−05 | 0.226711 | 1.17016 | 0.026761654 | −0.198801314 | 7 | 27185512 | + |
| cg25307665 | HOXA5 | 3.99E−08 | 0.000116957 | 0.330342 | 1.25731 | 0.038994644 | −0.289674714 | 7 | 27183694 | − |
| cg25390165 | HOXA5 | 4.09E−08 | 0.000118227 | 0.184959 | 1.13679 | 0.021833265 | −0.162189357 | 7 | 27184188 | + |
| cg25506432 | HOXA5 | 4.71E−09 | 2.63E−05 | 0.201534 | 1.14992 | 0.023789644 | −0.176723743 | 7 | 27184065 | − |
| cg25866143 | HOXA5 | 3.36E−10 | 4.16E−06 | 0.276725 | 1.21144 | 0.032665487 | −0.242658371 | 7 | 27183262 | + |
| cg26023912 | HOXA5 | 5.97E−08 | 0.000163623 | 0.214574 | 1.16036 | 0.025329038 | −0.188158357 | 7 | 27184369 | + |
| cg27151303 | N/A | 1.59E−06 | 0.001746174 | 0.166997 | 1.12272 | 0.019712871 | −0.146438143 | 7 | 27184821 | − |
| cg27355653 | LOC254559 | 2.56E−06 | 0.002409637 | 0.195894 | 1.14543 | 0.023123958 | −0.171777571 | 15 | 89940260 | − |
| cg27409910 | WT1 | 2.88E−09 | 1.91E−05 | 0.17705 | 1.13057 | 0.020899496 | −0.155253871 | 11 | 32454216 | + |

### REFERENCES

Baujat G, Cormier-Daire V. 2007. Sotos syndrome. Orphanet J Rare Dis 2:36.
Chen Y-a, Lemire M, Choufani S, Butcher D T, Grafodatskaya D, Zanke B W, Gallinger S, Hudson T J, Weksberg R. 2013a. Discovery of cross-reactive probes and polymorphic CpGs in the Illumina Infinium HumanMethylation450 microarray. Epigenetics 8(2):203-209.
Chen Y A, Lemire M, Choufani S, Butcher D T, Grafodatskaya D, Zanke B W, Gallinger S, Hudson T J, Weksberg R. 2013b. Discovery of cross-reactive probes and polymorphic CpGs in the Illumina Infinium HumanMethylation450 microarray. Epigenetics 8(2).
Gibson W T, Hood R L, Zhan S H, Bulman D E, Fejes A P, Moore R, Mungall A J, Eydoux P, Babul-Hirji R, An J and others. 2012. Mutations in EZH2 cause Weaver syndrome. Am J Hum Genet 90(1):110-8.
Harris R A, Wang T, Coarfa C, Nagarajan R P, Hong C, Downey S L, Johnson B E, Fouse S D, Delaney A, Zhao Y, Olshen A, Ballinger T, Zhou X, Forsberg K J, Gu J, Echipare L, O'Geen H, Lister R, Pelizzola M, Xi Y, Epstein C B, Bernstein B E, Hawkins R D, Ren B, Chung W Y, Gu H, Bock C, Gnirke A, Zhang M Q, Haussler D, Ecker J R, Li W, Farnham P J, Waterland R A, Meissner A, Marra M A, Hirst M, Milosavljevic A, Costello J F. 2010. Comparison of sequencing-based methods to profile DNA methylation and identification of monoallelic epigenetic modifications. Nat Biotechnol. Oct;28(10):1097-105.
Hirst M. 2013 Epigenomics: sequencing the methylome. Methods Mol Biol, 973:39-54. Huang da W, Sherman B T, Lempicki R A. 2009. Systematic and integrative analysis of large gene lists using DAVID bioinformatics resources. Nat Protoc 4(1):44-57.
Kurotaki N, Imaizumi K, Harada N, Masuno M, Kondoh T, Nagai T, Ohashi H, Naritomi K, Tsukahara M, Makita Y and others. 2002. Haploinsufficiency of NSD1 causes Sotos syndrome. Nat Genet 30(4):365-6.
Sotos J F, Dodge P R, Muirhead D, Crawford J D, Talbot N B. 1964. Cerebral Gigantism in Childhood. A Syndrome of Excessively Rapid Growth and Acromegalic Features and a Nonprogressive Neurologic Disorder. N Engl J Med 271:109-16.
Stec I, Wright T J, van Ommen G J, de Boer P A, van Haeringen A, Moorman A F, Altherr M R, den Dunnen J T. 1998. WHSC1, a 90 kb SET domain-containing gene, expressed in early development and homologous to a Drosophila dysmorphy gene maps in the Wolf-Hirschhorn syndrome critical region and is fused to IgH in t(4;14) multiple myeloma. Hum Mol Genet 7(7):1071-82.
Stevens M, Cheng J B, Li D, Xie M, Hong C, Maire C L, Ligon K L, Hirst M, Marra M A, Costello J F, Wang T. 2013 Estimating absolute methylation levels at single-CpG resolution from methylation enrichment and restriction enzyme sequencing methods. Genome Res. 2013 Sep; 23(9):1541-53.
Tatton-Brown K, Hanks S, Ruark E, Zachariou A, Duarte Sdel V, Ramsay E, Snape K, Murray A, Perdeaux E R, Seal S and others. 2011. Germline mutations in the oncogene EZH2 cause Weaver syndrome and increased human height. Oncotarget 2(12):1127-33.
Tatton-Brown K, Rahman N. 2007. Sotos syndrome. Eur J Hum Genet 15(3):264-71.
Thusberg J, Olatubosun A, Vihinen M. 2011. Performance of mutation pathogenicity prediction methods on missense variants. Hum Mutat 32(4):358-68.

1. A method of detecting and/or screening or Sotos syndrome (SS), or an increased likelihood of SS, in a human subject, comprising:
(a) determining a sample methylation profile from a sample comprising DNA from said subject, said sample profile comprising the methylation level of at least one, optionally at least 7, at least 10, at least 17, at least 25, at least 41, at least 57, at least 87, at least 124, at least 156, at least 220, at least 287, at least 399, at least 549, at least 726, or all CpG loci from (i) Table 1 and/or (ii) associated CpG loci residing within 300 nucleotides, optionally within 150 nucleotides, of the CpG loci of (l): and
determining the level of similarity of said sample profile to one or more control profiles, wherein (i) a high level of similarity of the sample profile to an SS specific control profile; (ii) a low level of similarity to a non-SS control profile; and/or (iii) a higher level of similarity to a SS specific control profile than to a non-SS control profile indicates the presence of, or an increased likelihood of, SS; and/or
(b) determining a sample methylation profile from a sample comprising DNA from said subject, said sample profile comprising the methylation level of at least one. optionally at least 6, at least 12, at least 17, at least 18, at least 24, at least 33, at least 49, at least 67, from Table 1 and
determining the level of similarity of said sample profile to one or more control profiles, wherein (i) a high level of similarity of the sample profile to a SS specific control profiles; (ii) a low level of similarity to a non-SS control profile: and/or (iii) a higher level of similarity to a SS control profile than a non-SS control profile indicates the presence of, or an increased likelihood of, SS.

2. The method of claim 1, wherein the selected CpG loci of (a) comprise CpG loci from Table 1 having an absolute SS delta-beta value ≧0.4, ≧0.42, ≧0.44, ≧0.46, ≧0.5, ≧0.52, ≧0.54, ≧0.56, ≧0.58, ≧0.6, ≧0.62, 0.64, or ≧0.66; and/or (ii) associated CpG loci residing within 300 nucleotides, optionally within 150 nucleotides, of the CpG loci of (i).

3.-18. (canceled)

19. The method of claim 1, wherein a high level of similarity to the control profile is indicated by a correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25: or wherein a higher level of similarity to the SS specific profile than to the non-SS control profile is indicated by a higher correlation value computed between the sample profile and the SS specific profile than an equivalent correlation value computed between the sample profile and the non-SS control profile, optionally wherein the correlation value is a correlation coefficient.

20. (canceled)

21. (canceled)

22. The method of claim 1, wherein methylation level is measured as a β-value,

23. The method of claim 22, wherein a Sotos Syndrome Score (SS score) is calculated according to the following formula:
SS score(B)=r(B, SS profile)−r(B, non-SS profile)
where r is a Pearson correlation coefficient, and B is a vector of DNA methylation levels across the selected CpG loci in the sample,

24.-29. (canceled)

30. The method of claim 1, wherein the sample is derived from blood, fibroblast tissue, buccal tissue, lymphoblastoid cell line, saliva or a prenatal sample, optionally a CVS, placenta, circulating fetal DNA and/or amniotic fluid sample.

31. The method of claim 1, wherein the human subject is a fetus.

32.-37. (canceled)

38. A method of determining a course of management for an individual with Sotos syndrome (SS), or an increased likelihood of SS, comprising:
a) identifying an individual with SS or an increased likelihood of SS, according to the method of claim 1; and
b) assigning a course of management for SS and/or symptoms of a SS, comprising i) testing for at least one medical condition associated with SS and ii) applying an appropriate medical intervention based on the results of the testing.

39. The method of claim 38, wherein the medical condition is selected from congenital heart defects, congenital renal abnormalities, hearing problems, vision problems, scoliosis, seizure activity, developmental/learning disabilities, behavioural problems and neuropsychological issues.

40. A kit for detecting and/or screening for Sotos syndrome, or an increased likelihood of SS, in a sample, comprising:
a) at least one detection agent for determining the methylation level of:
at least one, at least 7, at least 10, at least 17, at least 25, at least 41, at least 57, at least 87, at least 124, at least 156, at least 220, at least 287, at least 399, at least 549, at least 726, or all CpG loci selected from Table 1, and/or
ii) at least one, at least 6, at least 12, at least 17, at least 18, at least 24, at least 33, at least 49, at least 67, at least 72, at least 101, at least 127, at least 164, at least 212, at least 275, or all the genes from Table 1; and
b) instructions for use.

41. The kit according to claim 40, further comprising bisulfite conversion reagents, methylation-dependent restriction enzymes, methylation-sensitive restriction enzymes, PCR reagents, probes, primers, and/or a computer-readable medium that causes a comouter to compare methylation levels from a sample at the selected CpG loci to one or more control profiles and compute a correlation value between the sample and control profile.

42. (canceled)

43. A method of detecting and/or screening for Weaver syndrome (WVS), or an increased likelihood of WVS, in a human subject, comprising:
(a) determining a sample methylation profile from a sample comprising DNA from said subject, said sample profile comprising the methylation level of at least one, at least 3, at least 5, at least 10, at least 20, at least 30, at least 40, or all CpG loci from (i) Table 8 and/or (ii) associated CpG loci residing within 300 nucleotides, optionally within 150 nucleotides, of the CpG loci of (i); and
determining the level of similarity of said sample profile to one or more control profiles, wherein (i) a high level of similarity of the sample profile to an WVS specific control profile; (ii) a low level of similarity to a non-WVS control profile; and/or (iii) a higher level of similarity to a WVS specific control profile than to a non-WVS control profile indicates the presence of, or an increased likelihood of, WVS; and/or
(b) determining a sample methylation profile profilefrom a sample comprising DNA from said subject, said sample profile comprising the methylation level of at least one, optionally at least 2, at least 4, at least 6, or all the genes from Table 8; and
determining the level of similarity of said sample profile to one or more control profiles, wherein (i) a high level of similarity of the sample profile to a WVS specific control profile; (ii) a low level of similarity to a non-WVS control profile; and/or (iii) a higher level of similarity to a WVS control profile than a non-WVS control profile indicates the presence of or an increased likelihood of, WVS.

44.(canceled)

45. The method of claim 43, wherein a high level of similarity to the control profile is indicated by a correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25; or wherein a higher level of similarity to the WVS specific profile than to the non-WVS control profile is indicated by a higher correlation value computed between the sample profile and the WVS specific profile than an equivilent correlation value computed between the sample profile and the non-WVS control profile, optionally wherein the correlation value is a correlation coefficient.

46.(canceled)

47.(canceled)

48. The method of claim 43, wherein methylation level is measured as a β-value.

49. The method of claim 43, wherein a Weaver Syndrome Score (WVS score) is calculated according to the following formula:
WVS score(B)=r(B, WVS profile)−r(B, non-WVS profile)
where r is a Pearson correlation coefficient, and B is a vector of DNA methylation levels across the selected CpG loci in the sample.

50.-55. (canceled)

56. The method of claim 43, wherein the sample is derived from blood, fibroblast tissue, buccal tissue, lymphoblastoid cell line, saliva or a prenatal sample, optionally a CVS, placenta, circulating fetal DNA and/or amniotic fluid sample.

57. The method of claim 43, wherein the human subject is a fetus.

58.-63. (canceled)

64. A method of determining a course of management for an individual with Weaver syndrome (WVS), or an increased likelihood of WVS, comprising:
a) identifying an individual with WVS or an increased likelihood of WVS, according to the method of claim 43; and
b) assigning a course of management for WVS and/or symptoms of a WVS, comprising i) testing for at least one medical condition associated with SS and ii) applying an appropriate medical intervention based on the results of the testing,

65. The method of claim 64, wherein the medical condition is selected from scoliosis, camptodactyly, tumor growth, developmental/learning disabilities, intellectual disabilities, hypotonia and ligamentous laxity.

66. A kit for detecting and/or screening for Weaver syndrome, or an increased likelihood of WVS, in a sample, comprising:
a) at least one detection agent for determining the methylation level of (i) at least one, at least 3, at least 5, at least 10, at least 20, at least 30, at least 40, or all CpG loci from Table 8, and/or (ii) at least one, optionally at least 2, at least 4, at least 6, or all the genes from Table 8; and
b) instructions for use,

67. The kit according to claim 66, further comprising bisulfite conversion reagents, methylation dependent restriction enzymes, methylation-sensitive restriction enzymes, PCR reagents, probes, primers, and/or a computer-readable medium that causes a computer to compare methylation levels from a sample at the selected CpG loci to one or more control profiles and commute a correlation value between the sample and control profile.

68. (canceled)
